(19) United States
(12) Patent Application Publication
(10) Pub. No.: US 2025/0051309 A1
(43) Pub. Date: Feb. 13, 2025

(54) MK2 INHIBITORS AND METHODS OF MAKING AND USING THE SAME

(71) Applicant: Gilead Sciences, Inc., Foster City, CA (US)

(72) Inventors: Jason E. Anesini, San Francisco, CA (US); James L. Bachman, Seattle, WA (US); Alexander J. Burckle, Hayward, CA (US); Christopher T. Clark, Seattle, WA (US); Kerry E. Jones, Sunnyvale, CA (US); Zachary A. Kasun, Seattle, WA (US); Jennifer A. Loyer-Drew, Seattle, WA (US); Gregory T. Notte, Redwood City, CA (US); Michael S. Sangi, San Mateo, CA (US); Adam J. Schrier, Redwood City, CA (US); Joshua J. Van Veldhuizen, Seattle, WA (US)

(21) Appl. No.: 18/754,794

(22) Filed: Jun. 26, 2024

Related U.S. Application Data
(60) Provisional application No. 63/511,147, filed on Jun. 29, 2023.

(51) Int. Cl. C07D 401/14; A61K 31/444; A61K 31/497; A61K 31/501; A61K 31/5365; A61K 31/5383; C07D 401/04; C07D 487/04; C07D 498/04

(52) U.S. Cl. CPC C07D 401/14; A61K 31/444; A61K 31/497; A61K 31/501; A61K 31/5365; A61K 31/5383; C07D 401/04; C07D 487/04; C07D 498/04

(57) ABSTRACT

The present disclosure relates generally to certain compounds, pharmaceutical compositions comprising said compounds, and methods of making and using said compounds and pharmaceutical compositions. The compounds and compositions provided herein may be used for the treatment or prevention of an autoimmune disease and/or inflammatory condition, including rheumatoid arthritis.

### CROSS-REFERENCE TO RELATED APPLICATIONS

This application claims the benefit under 35 U.S.C. § 119(e) to U.S. Provisional Patent Application No. 63/511,147, filed Jun. 29, 2023, the contents of which is incorporated herein in its entirety.

### FIELD

This disclosure relates generally to novel MK2 inhibiting compounds, pharmaceutical compositions comprising said compounds, and methods of making and using said compounds and pharmaceutical compositions. In some embodiments, the novel MK2 inhibiting compounds provided herein may be used in the treatment of certain diseases and disorders, including, but not limited to, inflammatory conditions.

### BACKGROUND

Mitogen-activated protein kinases (MAPK) are a conserved family of enzymes that relay and propagate external stimuli, using phosphorylation cascades to generate a coordinated cellular response to the environment. The MAPK are proline-directed serine/threonine-specific protein kinases that regulate cellular activities, such as gene expression, mitosis, differentiation, and cell survival/apoptosis. To date, four distinct classes of mammalian MAPK have been identified: the extracellular signaling kinases (ERK1 and 2), the c-junN-terminal kinase-1 (JNK1-3), the p38 MAPK (ρ38α, β, γ, and δ), and ERK5. The MAPK are activated by the dual phosphorylation of Thr and Tyr residues within a TXY activation motif by coordinated dual-specificity MAPKK, where X is Glu, Pro, and Gly in ERK, JNK, and p38 MAPK, respectively. MAPK are 60-70% identical to each other, yet differ in their activation loop sequences and sizes. The activation loop is adjacent to the enzyme-active site, and its phosphorylation allows the enzyme to reposition active-site residues into the optimal orientation for substrate binding and catalysis. Downstream substrates of MAPK include mitogen-activated protein-kinase-activated protein (MAPKAP) kinases and transcription factors, the phosphorylation of which, either directly or indirectly, regulates gene expression at several points, including transcription, nuclear export, and mRNA stability and translation. The cellular consequences of MAPK activation include inflammation, apoptosis, differentiation, and proliferation.

Distinct genes encode four p38 MAPK in humans: ρ38α, β, γ, and δ. Significant amino acid sequence homology is observed among the 4 isoforms, with 60-75 overall sequence identity and >90% identity within the kinase domains. Tissue-selective expression is observed, with ρ38γ found predominantly in skeletal muscle, ρ38δ in the testes, pancreas, and small intestine. In contrast, ρ38a and β are more ubiquitously expressed.

p38 MAPK is the major isoform involved in the immune and inflammatory response. As such its function is critical for the production and activity of multiple proinflammatory cytokines, including TNFa, IL-1, IL-6, and IL-8, in cells such as macrophages, monocytes, synovial cells, and endothelial cells. p38 MAPK is also responsible for the induction of key inflammatory enzymes such as COX2 and iNOS, the major sources of eicosanoids and nitric oxide at sites of inflammation, respectively. Additionally, the p38 MAPK pathway regulates the expression of matrix metalloproteinases (MMP), including MMP2, MMP9, and MMP13.

The use of selective and potent inhibitors has facilitated the discovery of several families of p38 MAPK substrates, including transcription factors, MAPKAP kinases, and other enzymes. p38 MAPK can directly phosphorylate several transcription factors, such as myocyte—specific enhancer binding factor 2C (MEF2C), CHOP, peroxisome proliferator-activated receptor (PPAR) a, PPAR γ co-activator 1 and p53. These transcription factors are involved in cellular functions such as apoptosis, gluconeogenesis, and synthesis of enzymes involved in fatty acid oxidation. p38 MAPK is also involved in the direct or indirect phosphorylation of enzyme substrates, such as cytosolic phospholipase A2, and the Cdc25 phosphatases, which are involved in the activation of cy clin-dependent protein kinase activity and cell-cycle regulation. Therefore in addition to its role in the inflammatory response, p38 MAPK has other functions associated with normal and abnormal cell growth and survival as well as cellular function and homeostasis. The MAPKAP kinases (MK2, MK3, and PRAK) are selectively phosphorylated by p38 MAPK, while the phosphorylation of MSK1/2, MNK1/2, and RSKb is catalyzed by both p38 MAPK and ERK.

MK2, MK3, and PRAK, once phosphorylated and activated by p38 MAPK, share similar substrate specificities. All of these kinases can phosphorylate the small heat-shock protein Hsp27. Studies have shown that the PRAK- and MK3-deficient mice do not display any resistance to endotoxic shock or a decrease in lipopolysaccharide-(LPS)-induced cytokine production. In contrast, MK2-deficient mice show a resistance to endotoxic shock and an impaired inflammatory response, as well as a significantly decreased production of cytokines such as TNFa, IFNy and IL-6. Thus, the p38/MK2 axis is important for mediating pro-inflammatory responses.

The p38:MK2 complex is very stable with a Kd of 6 nM. The binding affinity of p38 for MK2 is driven by the C-terminal domain of MK2 containing several positively charged amino acid residues. Crystallographic studies of the p38:MK2 complex demonstrated that the C-terminal region of MK2 wraps around p38a and binds to the negatively charged ED binding site. The tight binding of p38 to MK2 may give rise to conformational changes providing additional binding pockets for inhibitors that would specifically be dependent upon the p38:MK2 interaction. Taken together, these two studies suggests that selective p38/MK2 axis blockade is achievable with small molecule inhibitors. In comparison to traditional p38 MAPK inhibitors these p38/MK2 inhibitors should retain or enhance potency and exhibit improved safety features in animal models of disease or in human clinical settings.

The p38/MK2 role in the regulation of inflammatory cytokines (TNFa, IL-1β, IL-6) and enzymes responsible for inflammation (COX-2, iNOS, and MMPs) makes it an attractive drug target. Several classical p38 MAPK inhibitors have progressed to testing in clinical trials. Some of these candidates have failed, for safety or other reasons, but several have reported clinical data in diseases such as rheumatoid arthritis, pain, Crohn's disease, acute coronary syndrome, multiple myeloma and chronic obstructive pulmonary disease. In addition to these diseases several IL-1β mediated diseases could be impacted by a p38 inhibitor based upon the key role for the p38 MAPK pathway in the biosynthesis and activity of this cytokine. These diseases include the family of cryopyrin associated periodic disorders (CAPS), chronic gout, diabetes, Still's disease, Familial Mediterranean Fever among others.

### SUMMARY

In one embodiment, provided herein is a compound of Formula (I)

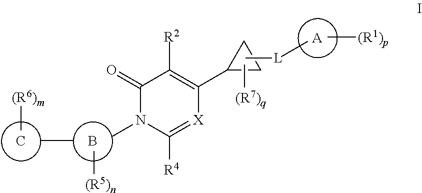

or a pharmaceutically acceptable salt thereof, wherein
ring \textcircled{A} is C₆₋₁₀ aryl, 5- to 10-membered heteroaryl, C₃₋₁₀ cycloalkyl, or 4- to 10-membered heterocyclyl;
each R¹ is independently halogen, —CD₃, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g};
each R^{1a} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, two R^{1a} groups taken together with the nitrogen atom to which they are attached form a 4- to 10-membered heterocyclyl, wherein each alkyl, alkenyl, alkynyl, cycloalkyl, heterocyclyl, aryl, and heteroaryl is optionally substituted with one to three R^{1g};
each R^{1g} is independently —CN, C₁₋₆ alkyl, C₂₋₆ alkynyl, C₃₋₁₀ cycloalkyl, 4- to 10-membered heterocyclyl, C₆₋₁₀ aryl, 5- to 10-membered heteroaryl, —OR^{1h}, —N(R^{1h})(R^{1h}), —C(O)N(R^{1h})(R^{1h}), —N(R^{1h})C(O)—R^{1h}, —N(R^{1h})C(O)O—R^{1h}, —N(R^{1h})C(O)N(R^{1h})(R^{1h}), —OC(O)N(R^{1h})(R^{1h}), —N(R^{1a})S(O)₂(R^{1a}), —S(O)₂R^{1h}, or S(O)₂N(R^{1a})(R^{1a}), wherein each alkyl, alkynyl, cycloalkyl, heterocyclyl, aryl, and heteroaryl is optionally substituted with one to three R¹ⁱ;
each R^{1h} is independently H, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, or two R^{1h} groups taken together with the nitrogen atom to which they are attached form a 4- to 10-membered heterocyclyl, wherein each alkyl, alkynyl, cycloalkyl, heterocyclyl, aryl and heteroaryl may be further substituted with one to three R¹ⁱ;
each R¹ⁱ is independently halogen, —OH, —NH₂, —CN, C₁₋₆ alkyl, C₁₋₆ haloalkyl, C₁₋₆ hydroxyalkyl, C₃₋₆ cycloalkyl, C₃₋₆ halocycloalkyl, C₁₋₆ alkoxy, C₁₋₆ haloalkoxy, or —O(C₃₋₆ cycloalkyl);
L is a bond, —O—, —NR^{La}—, —C(O)NR^{La}—, —C(O)—, or C₁₋₃ alkylene, wherein the alkylene is optionally substituted with one to three R^{Lb};
each R^{La} is independently H, C₁₋₆ alkyl, C₁₋₆ haloalkyl, or C₃₋₁₀ cycloalkyl;
each R^{Lb} is independently halogen, —OH, —OCH₃, —NH₂, or —CN;
each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy;
or R^{Y} and R¹ join together to form a C₃₋₆ cycloalkyl or 4- to 6-membered heterocyclyl, wherein each cycloalkyl and heterocyclyl is optionally substituted with one to three halogen;
X is CR³ or N;
R² is —H, halogen, C₁₋₆ alkyl, or C₃₋₆ cycloalkyl, wherein each alkyl and cycloalkyl is optionally substituted with one to three halogen;
R³ is —H, halogen, —CN, C₁₋₆ alkyl, C₁₋₆ haloalkyl, C₁₋₆ hydroxyalkyl, C₁₋₆ alkoxy, C₁₋₆ haloalkoxy, or —O(C₃₋₆ cycloalkyl);
R⁴ is —H, —CN, C₁₋₆ alkyl, C₂₋₆ alkynyl, or C₃₋₁₀ cycloalkyl, wherein each alkyl, alkynyl, and cycloalkyl is optionally substituted with one to three R^{4a1};
each R^{4a1} is independently halogen, —CN, —OR^{4a2}, or —NR^{4a2}R^{4a2};
each R^{4a2} is independently H, C₁₋₆ alkyl, C₁₋₆ haloalkyl, C₃₋₆ cycloalkyl, —C(O)C₁₋₆ alkyl, —C(O)O(C₁₋₆ alkyl), or —C(O)N(R^{4a3})₂;
each R^{4a3} is independently C₁₋₆ alkyl;
ring \textcircled{B} is C₆₋₁₀ aryl or 5- to 10-membered heteroaryl;
each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1}), —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one to three R^{5b1};
each R^{5a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with one to three R^{5b3};
each R^{5b1} is independently halogen, —CN, C₁₋₆ alkyl, C₂₋₆ alkynyl, C₁₋₆ haloalkyl, C₃₋₁₀ cycloalkyl, 4- to 10-membered heterocyclyl, —OR^{5b2}, or —N(R^{5b2})₂, wherein each alkyl, alkynyl, haloalkyl, cycloalkyl, and heterocycle is optionally substituted with one to three R^{5b3};
each R^{5b2} is independently H, C₁₋₆ alkyl, or C₃₋₁₀ cycloalkyl, wherein each alkyl and cycloalkyl is optionally substituted with one or two oxo and is further optionally substituted with one to three R^{5b3}.
each R^{5b3} is independently halogen, —CN, —OH, —NH₂, C₁₋₆ alkyl, C₁₋₆ haloalkyl, C₁₋₆ hydroxyalkyl, C₁₋₆ alkoxy, C₁₋₆ haloalkoxy, C₃₋₆ cycloalkyl, C₃₋₆ halocycloalkyl, or —O(C₃₋₆ cycloalkyl);
ring \textcircled{C} is C₆₋₁₀ aryl, 5- to 10-membered heteroaryl, C₃₋₈ cycloalkyl, or 4- to 10-membered heterocyclyl;
each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N═S(O)N(R^{6a1})₂R^{6a1}, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further optionally substituted with one to three R^{6b1};
each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl, wherein each alkyl, alkenyl, cycloalkyl, heterocyclyl, aryl, and heteroaryl is optionally substituted with one to three R^{6b1};
each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is optionally substituted with one to three R^{6c1};
each R^{6b2} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, wherein each alkyl, alkenyl, cycloalkyl, heterocyclyl, aryl, and heteroaryl is optionally substituted with one to three R^{6c2};
each R^{6c1} and R^{6c2} is independently oxo, halogen, —NO₂, —N₃, —CN, —OH, R^{6d1}, —O(R^{6d1}), —OC(O)(R^{6d1}), —NH₂, —NH(R^{6d1}), —N(R^{6d1})₂, —C(O)(R^{6d1}), —C(O)O(R^{6d1}), —C(O)NH₂, —C(O)NH(R^{6d1}), —C(O)N(R^{6d1})₂, —NHC(O)(R^{6d1}), —NHC(O)O(R^{6d1}), —NHC(O)NH(R^{6d1}), —SH, —S(R^{6d1}), —NHS(O)(R^{6d1}), —N(R^{6d1})(S(O)(R^{6d1}), —S(O)N(R^{6d1})₂, —S(O)(R^{6d1}), —S(O)(NH)(R^{6d1}), —S(O)₂(R^{6d1}), —S(O)₂NH(R^{6d1}), or —S(O)₂N(R^{6d1})₂;
each R^{6d1} is independently C₁₋₆ alkyl, C₃₋₁₀ cycloalkyl, 4- to 10-membered heterocyclyl, C₆₋₁₀ aryl, and 5- to 10-membered heteroaryl, wherein each alkyl, alkenyl, alkynyl, cycloalkyl, heterocyclyl aryl, and heteroaryl is optionally substituted with one to three R^{6e1};
each R^{6e1} is independently oxo, halogen, —NO₂, —N₃, —CN, —OH, —NH₂, methyl, or halomethyl; wherein each heterocyclyl and heteroaryl independently has one to four ring heteroatoms each independently selected from N, O, and S; and each m, n, p and q is independently 0, 1, 2 or 3.

In one embodiment, provided herein is a pharmaceutical composition comprising a compound provided herein, or a pharmaceutically acceptable salt thereof, and a pharmaceutically acceptable excipient or carrier.

In one embodiment, provided herein is a method of inhibiting p38 MAP kinase activity in a subject in need thereof, comprising administering to the subject a therapeutically effective amount of a compound provided herein, or a pharmaceutically acceptable salt thereof, or a therapeutically effective amount of a pharmaceutical composition provided herein.

In one embodiment, provided herein is a method of inhibiting MK2 activity in a subject in need thereof, comprising administering to the subject a therapeutically effective amount of a compound provided herein, or a pharmaceutically acceptable salt thereof, or a therapeutically effective amount of a pharmaceutical composition provided herein.

In one embodiment, provided herein is a method of treating a p38 MAP kinase-mediated disease or disorder in a subject in need thereof, comprising administering to the subject a therapeutically effective amount of a compound provided herein, or a pharmaceutically acceptable salt thereof, or a therapeutically effective amount of a pharmaceutical composition provided herein.

In one embodiment, provided herein is a method of treating a MK2-mediated disease or disorder in a subject in need thereof, comprising administering to the subject a therapeutically effective amount of a compound provided herein, or a pharmaceutically acceptable salt thereof, or a therapeutically effective amount of a pharmaceutical composition provided herein.

In one embodiment, provided herein is a method of treating an inflammatory condition in a subject in need thereof, comprising administering to the subject a therapeutically effective amount of a compound provided herein, or a pharmaceutically acceptable salt thereof, or a therapeutically effective amount of a pharmaceutical composition provided herein.

In one embodiment, provided herein is a method of treating an autoimmune disorder, a chronic inflammatory disorder, an acute inflammatory disorder, an auto-inflammatory disorder, a fibrotic disorder, a metabolic disorder, a neoplastic disorder, a cardiovascular disorder, or a cerebrovascular disorder in a subject in need thereof, comprising administering to the subject a therapeutically effective amount of a compound provided herein, or a pharmaceutically acceptable salt thereof, or a therapeutically effective amount of a pharmaceutical composition provided herein.

In one embodiment, provided herein is a method of treating rheumatoid arthritis in a subject in need thereof, comprising administering to the subject a therapeutically effective amount of a compound provided herein, or a pharmaceutically acceptable salt thereof, or a therapeutically effective amount of a pharmaceutical composition provided herein.

In one embodiment, provided herein is a compound provided herein, or a pharmaceutically acceptable salt thereof, or a pharmaceutical composition provided herein for use in therapy.

In one embodiment, provided herein is a compound provided herein, or a pharmaceutically acceptable salt thereof, or a pharmaceutical composition provided herein for use in a method of inhibiting p38 MAP kinase activity in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein, or a pharmaceutically acceptable salt thereof, or a pharmaceutical composition provided herein for use in a method of inhibiting MK2 activity in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein, or a pharmaceutically acceptable salt thereof, or a pharmaceutical composition provided herein for use in a method of treating a p38 MAP kinase-mediated in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein, or a pharmaceutically acceptable salt thereof, or a pharmaceutical composition provided herein for use in a method of treating a MK2-mediated disease or disorder in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein, or a pharmaceutically acceptable salt thereof, or a pharmaceutical composition provided herein for use in a method of treating an inflammatory condition in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein, or a pharmaceutically acceptable salt thereof, or a pharmaceutical composition provided herein for use in a method of treating an autoimmune disorder, a chronic inflammatory disorder, an acute inflammatory disorder, an auto-inflammatory disorder, a fibrotic disorder, a metabolic disorder, a neoplastic disorder, a cardiovascular disorder, or a cerebrovascular disorder in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein, or a pharmaceutically acceptable salt thereof, or a pharmaceutical composition provided herein for use in a method of treating rheumatoid arthritis in a subject in need thereof, comprising administering to the subject a therapeutically effective amount of the compound, or a pharmaceutically acceptable salt thereof, or a therapeutically effective amount of the pharmaceutical composition.

### DETAILED DESCRIPTION

### I. Definitions

The description below is made with the understanding that the present disclosure is to be considered as an exemplification of the claimed subject matter, and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

Unless defined otherwise, all technical and scientific terms used herein have the same meaning as commonly understood by one of ordinary skill in the art. It must be noted that as used herein and in the appended claims, the singular forms “a”, “and”, and “the” include plural referents unless the context clearly dictates otherwise. Thus, e.g., reference to “the compound” includes a plurality of such compounds and reference to “the assay” includes reference to one or more assays and equivalents thereof known to those skilled in the art, and so forth.

As used in the present disclosure, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. A solid line coming out of the center of a ring (including but not limited to a fused, bridged or spirocyclic ring system) indicates that the point of attachment for a substituent on the ring can be at any ring atom. For example, R^{aa} in the below structure can be attached to any of the five carbon ring atoms or R^{aa} can replace the hydrogen attached to the nitrogen ring atom:

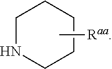

As another example, R^{aa} in the below structure:

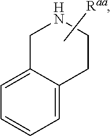

R^{aa} can be attached to any of the numbered positions shown below:

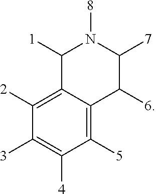

A solid line coming out of the center of a ring (including but not limited to a fused, bridged, or spirocyclic ring system), where a wavy line is drawn through a line, indicates that the point of attachment for the ring system to the rest of the compound can be at any ring atom of the fused, bridged, or spirocyclic ring system. For example, in the below structure:

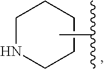

the monocyclic heterocyclyl can be attached to the rest of the compound at any of the numbered positions shown below:

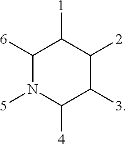

As another example, in the below fused bicyclic heterocyclic structure,

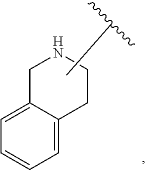

the fused bicyclic heterocyclyl can be attached to the rest of the compound at any of the eight numbered positions shown below:

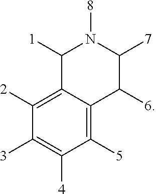

The prefix “Cᵤ₋ᵥ” indicates that the following group has from u to v carbon atoms. For example, “C₁₋₆ alkyl” indicates that the alkyl group has from 1 to 6 carbon atoms. Likewise, the term “x-y membered” rings, wherein x and y are numerical ranges, such as “3 to 12-membered heterocyclyl”, refers to a ring containing x-y atoms (i.e., 3-12), of which up to 80% may be heteroatoms, such as N, O, S, P, and the remaining atoms are carbon, unless specified otherwise.

Also, certain commonly used alternative chemical names may or may not be used. For example, a divalent group such as a divalent “alkyl” group, a divalent “aryl” group, etc., may also be referred to as an “alkylene” group or an “alkylenyl” group, or “alkylyl” group, an “arylene” group or an “arylenyl” group, or arylyl group, respectively.

“A compound disclosed herein” or “a compound of the present disclosure” or “a compound provided herein” or “a compound described herein” refers to the compounds of Formula I, II, III, IV, V, VI, or VII. Also included are the specific MK2 inhibitors of the Examples.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±10%. Also, the term “about X” includes description of “X”.

“Alkyl” refers to an unbranched or branched saturated hydrocarbon chain. As used herein, alkyl may have, for example, 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), 1 to 4 carbon atoms (i.e., C₁₋₄ alkyl), 1 to 3 carbon atoms (i.e., C₁₋₃ alkyl), or 1 to 2 carbon atoms (i.e., C₁₋₂ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Alkenyl” refers to an aliphatic group containing at least one carbon-carbon double bond and may have, for example, from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an aliphatic group containing at least one carbon-carbon triple bond and may have, for example, from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkylene” refers to a divalent and unbranched saturated hydrocarbon chain. As used herein, alkylene may have, for example, 1 to 20 carbon atoms (i.e., C₁₋₂₀ alkylene), 1 to 12 carbon atoms (i.e., C₁₋₁₂ alkylene), 1 to 8 carbon atoms (i.e., C₁₋₈ alkylene), 1 to 6 carbon atoms (i.e., C₁₋₆ alkylene), 1 to 4 carbon atoms (i.e., C₁₋₄ alkylene), 1 to 3 carbon atoms (i.e., C₁₋₃ alkylene), or 1 to 2 carbon atoms (i.e., C₁₋₂ alkylene). Examples of alkylene groups include methylene, ethylene, propylene, butylene, pentylene, and hexylene. In some embodiments, an alkylene is optionally substituted with an alkyl group. Examples of substituted alkylene groups include —CH(CH₃)CH₂—, —CH₂CH(CH₃)—, —CH₂CH(CH₂CH₃)—, —CH₂C(CH₃)₂—, —C(CH₃)₂CH₂—, —CH(CH₃)CH(CH₃)—, —CH₂C(CH₂CH₃)(CH₃)—, and —CH₂C(CH₂CH₃)₂.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy. “Haloalkoxy” refers to an alkoxy group as defined above, wherein one or more hydrogen atoms are replaced by a halogen.

“Acyl” refers to a group —C(═O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cylcohexylcarbonyl, cyclohexylmethyl-carbonyl, and benzoyl.

“Amido” refers to both a “C-amido” group which refers to the group —C(═O)NR^{y}R^{z} and an “N-amido” group which refers to the group —NR^{y}C(═O)R^{z}, wherein R^{y} and R^{z} are independently selected from the group consisting of hydrogen, alkyl, aryl, haloalkyl, heteroaryl, cycloalkyl, or heterocyclyl; each of which may be optionally substituted.

“Amino” refers to the group —NR^{y}R^{z} wherein R^{y} and R^{z} are independently selected from the group consisting of hydrogen, alkyl, haloalkyl, aryl, heteroaryl, cycloalkyl, or heterocyclyl; each of which may be optionally substituted.

“Aryl” as used herein refers to a single all carbon aromatic ring or a multiple condensed all-carbon ring system wherein at least one of the rings is aromatic. For example, in some embodiments, an aryl group may have 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Aryl includes a phenyl radical. The ring or ring system is optionally substituted with one or more (e.g., 1, 2 or 3) oxo groups. The rings of the multiple condensed ring system can be connected to each other via fused, spiro and bridged bonds when allowed by valency requirements. Examples of aryl groups include phenyl, naphthyl, fluorenyl, and anthryl. Aryl, however, does not encompass or overlap in any way with heteroaryl defined below. If one or more aryl groups are fused with a heteroaryl ring, the resulting ring system is heteroaryl.

“Cyano” or “carbonitrile” refers to the group —CN.

“Cycloalkyl” refers to a saturated or partially saturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl may have, for example, from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl. Examples of cycloalkyl groups having multiple ring systems include bicyclo[3.1.0]hexane, spiro[2.4]heptane, bicyclo[1.1.1]pentane and bicyclo[2.2.2]octane.

“Cycloalkoxy” refers to the group “cycloalkyl-O—”. Examples of cycloalkoxy groups include but are not limited to:

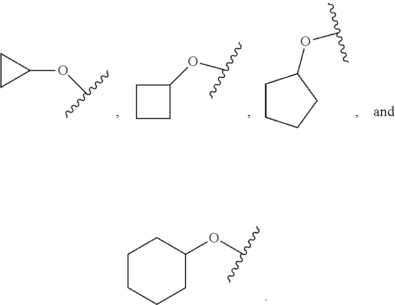

“Bridged” refers to a ring fusion wherein non-adjacent atoms on a ring are joined by a divalent substituent, such as an alkylenyl group, an alkylenyl group containing one or two heteroatoms, or a single heteroatom. Quinuclidinyl and adamantanyl are examples of bridged ring systems.

The term “fused” refers to a ring which is bound to an adjacent ring by bonds to adjacent atoms. Examples of fused groups include naphthalene and phenalene.

“Spiro” refers to a ring substituent which is joined by two bonds at the same carbon atom. Examples of spiro groups include 1,1-diethylcyclopentane, dimethyl-dioxolane, and 4-benzyl-4-methylpiperidine, wherein the cyclopentane and piperidine, respectively, are the spiro substituents.

“Halogen” or “halo” includes fluoro, chloro, bromo, and iodo.

“Haloalkyl” refers to an alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkylene” refers to a divalent and unbranched saturated hydrocarbon chain having one, two, or three heteroatoms selected from NH, O, or S. As used herein, a heteroalkylene may have, for example, 1 to 20 carbon atoms and one, two, or three heteroatoms selected from NH, O, and S (i.e., C₁₋₂₀ heteroalkylene); 1 to 8 carbon atoms and one, two, or three heteroatoms selected from NH, O, and S (i.e., C₁₋₈ heteroalkylene); 1 to 6 carbon atoms and one, two, or three heteroatoms selected from NH, O, and S S (i.e., C₁₋₆ heteroalkylene); 1 to 4 carbon atoms and one, two, or three heteroatoms selected from NH, O, and S (i.e., C₁₋₄ heteroalkylene); 1 to 3 carbon atoms and one, two, or three heteroatoms selected from NH, O, and S (i.e., C₁₋₃ heteroalkylene); or 1 to 2 carbon atoms and one, two, or three heteroatoms selected from NH, O, and S (i.e., C₁₋₃ heteroalkylene). For example, —CH₂O— is a C₁ heteroalkylene and —CH₂SCH₂— is a C₂ heteroalkylene. Examples of heteroalkylene groups include —CH₂CH₂OCH₂—, —CH₂SCH₂OCH₂—, —CH₂O—, and —CH₂NHCH₂—. In some embodiments, a heteroalkylene is optionally substituted with an alkyl group. Examples of substituted heteroalkylene groups include —CH(CH₃)N(CH₃)CH₂—, —CH₂OCH(CH₃)—, —CH₂CH(CH₂CH₃)S—, —CH₂NHC(CH₃)₂—, —C(CH₃)₂SCH₂—, —CH(CH₃)N(CH₃)CH(CH₃)O—, —CH₂SC(CH₂CH₃)(CH₃)—, and —CH₂C(CH₂CH₃)₂NH—.

“Heteroaryl” refers to an aromatic group having a single ring, multiple rings, or multiple fused rings, with one or more ring heteroatoms of at least one aromatic ring independently selected from nitrogen, oxygen, and sulfur, wherein the nitrogen or sulfur can be oxidized. Thus, the term includes rings having one or more annular O, N, S, S(O), S(O)₂, and N-oxide groups. The ring or rings are optionally substituted with one or more (e.g., 1, 2 or 3) oxo groups. An example of an oxo-substituted heteroaryl group is pyridine-2(1H)-onyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. The other ring or rings may be aromatic or not aromatic (i.e., carbocycle) and may be saturated or partially saturated. As used herein, heteroaryl may include, for example, 1 to 20 carbon ring atoms (i.e., C₁₋₂₀ heteroaryl), 3 to 12 carbon ring atoms (i.e., C₃₋₁₂ heteroaryl), or 3 to 8 carbon ring atoms (i.e., C₃₋₈ heteroaryl); and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, and pyrazolyl. Heteroaryl does not encompass or overlap with aryl as defined above.

“Heterocyclyl” or “heterocyclic ring” or “heterocycle” refers to a non-aromatic cyclic alkyl group, with one or more ring heteroatoms independently selected from nitrogen, oxygen and sulfur, wherein the nitrogen or sulfur can be oxidized. Thus, the term includes rings having one or more annular O, N, S, S(O), S(O)₂, and N-oxide groups. The ring or rings are optionally substituted with one or more (e.g., 1, 2 or 3) oxo groups. As used herein, “heterocyclyl” or “heterocyclic ring” or “heterocycle” refer to rings that are saturated or partially saturated unless otherwise indicated, e.g., in some embodiments “heterocyclyl” or “heterocyclic ring” or “heterocycle” refers to rings that are partially saturated. A heterocyclyl may be a single ring or multiple rings wherein the multiple rings may be fused, bridged, or spiro. As used herein, heterocyclyl may have, for example, 2 to 20 carbon ring atoms (i.e., C₂₋₂₀ heterocyclyl), 2 to 12 carbon ring atoms (i.e., C₂₋₁₂ heterocyclyl), 2 to 10 carbon ring atoms (i.e., C₂₋₁₀ heterocyclyl), 2 to 8 carbon ring atoms (i.e., C₂₋₈ heterocyclyl), 3 to 12 carbon ring atoms (i.e., C₃₋₁₂ heterocyclyl), 3 to 8 carbon ring atoms (i.e., C₃₋₈ heterocyclyl), or 3 to 6 carbon ring atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocyclyl groups include pyrrolidinyl, piperidinyl, piperazinyl, oxetanyl, dioxolanyl, azetidinyl, and morpholinyl.

“Hydroxy” or “hydroxyl” refers to the group —OH.

“Oxo” refers to the group (═O) or (O).

“Sulfonyl” refers to the group —S(O)₂R^{bb}, where R^{bb} is alkyl, haloalkyl, heterocyclyl, cycloalkyl, heteroaryl, or aryl. Examples of sulfonyl are methylsulfonyl, ethylsulfonyl, phenylsulfonyl, and toluenesulfonyl.

Whenever the graphical representation of a group terminates in a singly bonded nitrogen atom, that group represents an —NH group unless otherwise indicated. Similarly, unless otherwise expressed, hydrogen atom(s) are implied and deemed present where necessary in view of the knowledge of one of skill in the art to complete valency or provide stability.

The terms “optional” or “optionally” mean that the subsequently described event or circumstance may or may not occur, and that the description includes instances where said event or circumstance occurs and instances in which it does not. Also, the term “optionally substituted” means that any one or more hydrogen atoms on the designated atom or group may or may not be replaced by a moiety other than hydrogen.

The term “substituted” means that any one or more hydrogen atoms on the designated atom or group is replaced with one or more substituents other than hydrogen, provided that the designated atom's normal valence is not exceeded. The one or more substituents may include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or any combination thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. For example, the term “substituted aryl” includes, but is not limited to, “alkylaryl.” Unless specified otherwise, where a group is described as optionally substituted, any substituents of the group are themselves unsubstituted.

In some embodiments, a substituted cycloalkyl, a substituted heterocyclyl, a substituted aryl, and/or a substituted heteroaryl includes a cycloalkyl, a heterocyclyl, an aryl, and/or a heteroaryl that has a substituent on the ring atom to which the cycloalkyl, heterocyclyl, aryl, and/or heteroaryl is attached to the rest of the compound. For example, in the below moiety, the cyclopropyl is substituted with a methyl group:

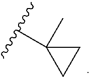 [SMILES: *C1(C)CC1]

The compounds of the embodiments disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

“Enantiomers” are a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

The compounds of the embodiments disclosed herein, or their pharmaceutically acceptable salts may contain one or more bonds with hindered rotation such that two separate atropisomers can be isolated, giving rise to enantiomers, diastereomers, and other stereoisomeric forms.

“Diastereoisomers” are stereoisomers that have at least two asymmetric atoms, or at least one stereogenic axis resulting from hindered rotation and one asymmetric atom, but which are not mirror-images of each other.

Some of the compounds provided herein exist as tautomeric isomers. Tautomeric isomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers. In some embodiments, the tautomer is interconvertible by migration of a hydrogen atom.

A “solvate” is formed by the interaction of a solvent and a compound. Solvates of salts of the compounds provided herein are also provided. Solvates may contain either stoichiometric or non-stoichiometric amounts of a solvent. Hydrates of the compounds provided herein are also provided.

Any formula or structure provided herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ²H, ³H, ¹³C and ¹⁴C are incorporated, are also provided herein. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The present disclosure also includes compounds of Formula I, II, III, IV, V, VI, or VII, in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I, II, III, IV, V, VI, or VII when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the present disclosure may have improved DMPK (drug metabolism and pharmacokinetics) properties, relating to absorption, distribution, metabolism and excretion (ADME). Substitution with heavier isotopes such as deuterium may afford certain therapeutic advantages resulting from greater metabolic stability, for example, increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The concentration of such a heavier isotope, specifically deuterium, may be defined by an isotopic enrichment factor. In the compounds of this disclosure, any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure, any atom specifically designated as a deuterium (D) is meant to represent deuterium.

In many cases, the compounds of this disclosure are capable of forming acid and/or base salts by virtue of the presence of amino and/or carboxyl groups or groups similar thereto.

The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, mono, di or tri cycloalkyl amines, mono, di or tri arylamines or mixed amines, and the like. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Treatment” or “treating” is an approach for obtaining beneficial or desired results including clinical results. Beneficial or desired clinical results may include one or more of the following: a) inhibiting the disease or condition (i.e., decreasing one or more symptoms resulting from the disease or condition, and/or diminishing the extent of the disease or condition); b) slowing or arresting the development of one or more clinical symptoms associated with the disease or condition (i.e., stabilizing the disease or condition, preventing or delaying the worsening or progression of the disease or condition, and/or preventing or delaying the spread (i.e., metastasis) of the disease or condition); and/or c) relieving the disease, that is, causing the regression of clinical symptoms (i.e., ameliorating the disease state, providing partial or total remission of the disease or condition, enhancing effect of another medication, delaying the progression of the disease, increasing the quality of life, and/or prolonging survival).

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or pharmaceutically acceptable salts, isomer, or a mixture thereof means an amount sufficient to effect treatment when administered to a subject, to provide a therapeutic benefit such as amelioration of symptoms or slowing of disease progression. The therapeutically effective amount may vary depending on the subject, and the disease or condition being treated, the weight and age of the subject, the severity of the disease or condition, and the manner of administering.

### II. Compounds

In one embodiment, provided herein is a compound of Formula I, II, III, IV, V, VI, or VII.

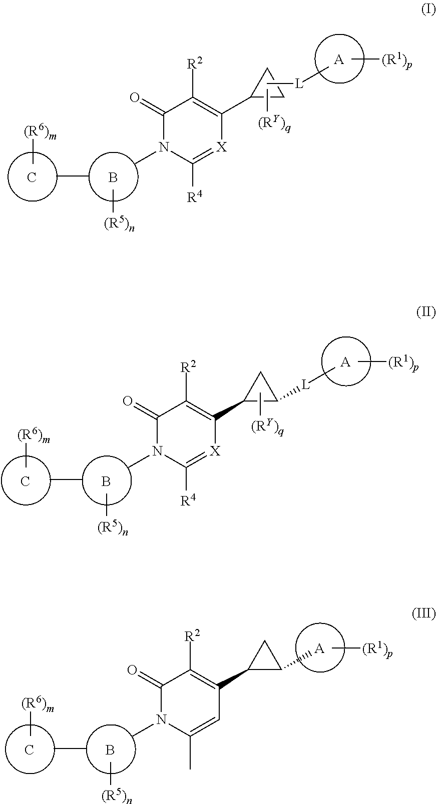

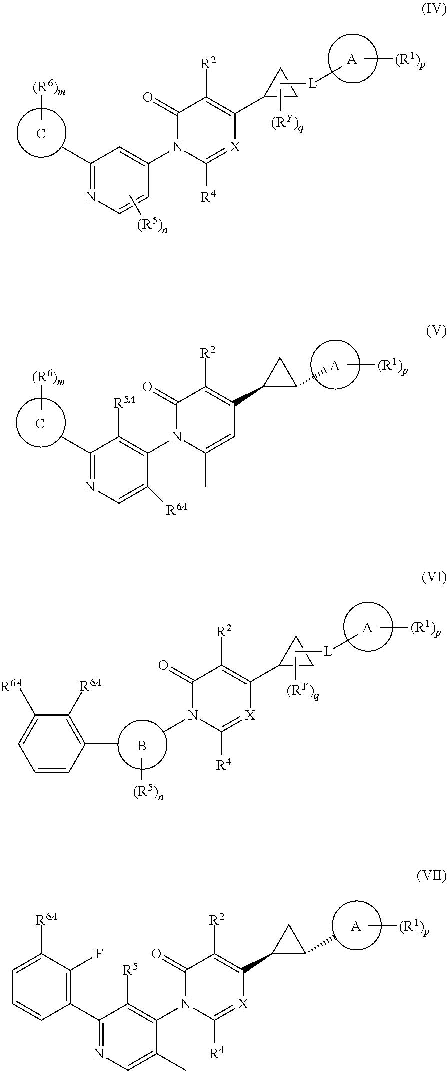

or a pharmaceutically acceptable salt thereof, wherein ring \textcircled{A}, R¹, L, R^{Y}, X, R², R³, R⁴, ring \textcircled{B}, R⁵, ring \textcircled{C}, R⁶, m, n, p, and q are as defined for the compounds described herein.

In one embodiment, provided herein is a compound of Formula (I)

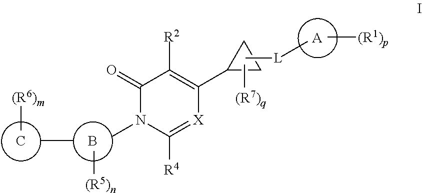

or a pharmaceutically acceptable salt thereof, wherein
ring \textcircled{A} is C₆₋₁₀ aryl, 5- to 10-membered heteroaryl, C₃₋₁₀ cycloalkyl, or 4- to 10-membered heterocyclyl;
each R¹ is independently halogen, —CD₃, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(Ra)(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g};
each R^{1a} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, two R^{1a} groups taken together with the nitrogen atom to which they are attached form a 4- to 10-membered heterocyclyl, wherein each alkyl, alkenyl, alkynyl, cycloalkyl, heterocyclyl, aryl, and heteroaryl is optionally substituted with one to three R^{1g};
each R^{1g} is independently —CN, C₁₋₆ alkyl, C₂₋₆ alkynyl, C₃₋₁₀ cycloalkyl, 4- to 10-membered heterocyclyl, C₆₋₁₀ aryl, 5- to 10-membered heteroaryl, —OR^{1h}, —N(R^{1h})(R^{1h}), —C(O)N(R^{1h})(R^{1h}), —N(R^{1h})C(O)—R^{1h}, —N(R^{1h})C(O)O—R^{1h}, —N(R^{1h})C(O)N(R^{1h})(R^{1h}), —OC(O)N(R^{1h})(R^{1h}), —N(R^{1a})S(O)₂(R^{1a}), —S(O)₂R^{1h}, or —S(O)₂N(R^{1a})(R^{1a}) wherein each alkyl, alkynyl, cycloalkyl, heterocyclyl, aryl, and heteroaryl is optionally substituted with one to three R¹ⁱ;
each R^{1h} is independently H, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, or two R^{1h} groups taken together with the nitrogen atom to which they are attached form a 4- to 10-membered heterocyclyl, wherein each alkyl, alkynyl, cycloalkyl, heterocyclyl, aryl and heteroaryl may be further substituted with one to three R¹ⁱ;
each R¹ⁱ is independently halogen, —OH, —NH₂, —CN, C₁₋₆ alkyl, C₁₋₆ haloalkyl, C₁₋₆ hydroxyalkyl, C₃₋₆ cycloalkyl, C₃₋₆ halocycloalkyl, C₁₋₆ alkoxy, C₁₋₆ haloalkoxy, or —O(C₃₋₆ cycloalkyl);
L is a bond, —O—, —NR^{La}—, —C(O)NR^{La}—, —C(O)—, or C₁₋₃ alkylene, wherein the alkylene is optionally substituted with one to three R^{Lb};
each R^{La} is independently H, C₁₋₆ alkyl, C₁₋₆ haloalkyl, or C₃₋₁₀ cycloalkyl; each R^{Lb} is independently halogen, —OH, —OCH₃, —NH₂, or —CN;
each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy;
or R^{Y} and R¹ join together to form a C₃₋₆ cycloalkyl or 4- to 6-membered heterocyclyl, wherein each cycloalkyl and heterocyclyl is optionally substituted with one to three halogen;
X is CR³ or N;
R² is H, halogen, C₁₋₆ alkyl, or C₃₋₆ cycloalkyl, wherein each alkyl and cycloalkyl is optionally substituted with one to three halogen;
R³ is H, halogen, —CN, C₁₋₆ alkyl, C₁₋₆ haloalkyl, C₁₋₆ hydroxyalkyl, C₁₋₆ alkoxy, C₁₋₆ haloalkoxy, or —O(C₃₋₆ cycloalkyl);
R⁴ is H, —CN, C₁₋₆ alkyl, C₂₋₆ alkynyl, or C₃₋₁₀ cycloalkyl, wherein each alkyl, alkynyl, and cycloalkyl is optionally substituted with one to three R^{4a1};
each R^{4a1} is independently halogen, —CN, —OR^{4a2}, or —NR^{4a2}R^{4a2};
each R^{4a2} is independently H, C₁₋₆ alkyl, C₁₋₆ haloalkyl, C₃₋₆ cycloalkyl, —C(O)C₁₋₆ alkyl, —C(O)O(C₁₋₆ alkyl), or —C(O)N(R^{4a3})₂;
each R^{4a3} is independently C₁₋₆ alkyl;
ring \textcircled{B} is C₆₋₁₀ aryl or 5- to 10-membered heteroaryl;
each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one to three R^{5b1};
each R^{5a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with one to three R^{5b3};
each R^{5b1} is independently halogen, —CN, C₁₋₆ alkyl, C₂₋₆ alkynyl, C₁₋₆ haloalkyl, C₃₋₁₀ cycloalkyl, 4- to 10-membered heterocyclyl, —OR¹², or —N(R^{5b2})₂, wherein each alkyl, alkynyl, haloalkyl, cycloalkyl, and heterocycle is optionally substituted with one to three R^{5b3};
each R^{5b2} is independently H, C₁₋₆ alkyl, or C₃₋₁₀ cycloalkyl, wherein each alkyl and cycloalkyl is optionally substituted with one or two oxo and is further optionally substituted with one to three R^{5b3};
each R^{5b3} is independently halogen, —CN, —OH, —NH₂, C₁₋₆ alkyl, C₁₋₆ haloalkyl, C₁₋₆ hydroxyalkyl, C₁₋₆ alkoxy, C₁₋₆ haloalkoxy, C₃₋₆ cycloalkyl, C₃₋₆ halocycloalkyl, or —O(C₃₋₆ cycloalkyl);
ring \textcircled{C} is C₆₋₁₀ aryl, 5- to 10-membered heteroaryl, C₃₋₈ cycloalkyl, or 4- to 10-membered heterocyclyl;
each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N═S(O)N(R^{6a1})₂R^{6a1}, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further optionally substituted with one to three R^{6b1};
each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl, wherein each alkyl, alkenyl, cycloalkyl, heterocyclyl, aryl, and heteroaryl is optionally substituted with one to three R^{6b1};
each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is optionally substituted with one to three R^{6c1};
each R^{6b2} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, wherein each alkyl, alkenyl, cycloalkyl, heterocyclyl, aryl, and heteroaryl is optionally substituted with one to three R^{6c2};
each R^{6c1} and R^{6c2} is independently oxo, halogen, —NO₂, —N₃, —CN, —OH, R^{6d1}, —O(R^{6d1}), —OC(O)(R^{6d1}), —NH₂, —NH(R^{6d1}), —N(R^{6d1})₂, —C(O)(R^{6d1}), —C(O)O(R^{6d1}), —C(O)NH₂, —C(O)NH(R^{6d1}), —C(O)N(R^{6d1})₂, —NHC(O)(R^{6d1}), —NHC(O)O(R^{6d1}), —NHC(O)NH(R^{6d1}), —SH, —S(R^{6d1}), —NHS(O)(R^{6d1}), —N(R^{6d1})(S(O)(R^{6d1}), —S(O)N(R^{6d1})₂, —S(O)(R^{6d1}), —S(O)(NH)(R^{6d1}), —S(O)₂ (R^{6d1}), —S(O)₂NH(R^{6d1}), or —S(O)₂N(R^{6d1})₂;
each R^{6d1} is independently C₁₋₆ alkyl, C₃₋₁₀ cycloalkyl, 4- to 10-membered heterocyclyl, C₆₋₁₀ aryl, or 5- to 10-membered heteroaryl, wherein each alkyl, alkenyl, alkynyl, cycloalkyl, heterocyclyl aryl, and heteroaryl is optionally substituted with one to three R^{6e1};
each R^{6e1} is independently oxo, halogen, —NO₂, —N₃, —CN, —OH, —NH₂, methyl, or halomethyl; wherein each heterocyclyl and heteroaryl independently has one to four ring heteroatoms each independently selected from N, O, and S; and
m is 0, 1, 2 or 3;
n is 0, 1, 2 or 3;
p is 0, 1, 2 or 3; and
q is 0, 1, 2 or 3.

In one embodiment, provided herein is a compound of Formula (I)

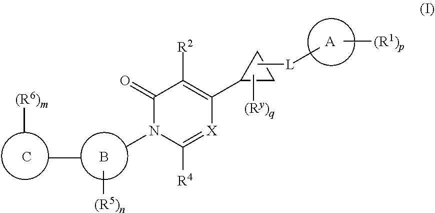

or a pharmaceutically acceptable salt thereof, wherein
ring \textcircled{A} is C₆₋₁₀ aryl, 5- to 10-membered heteroaryl, C₃₋₁₀ cycloalkyl, or 4- to 10-membered heterocyclyl;
each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g};
each R^{1a} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, two R^{1a} groups taken together with the nitrogen atom to which they are attached form a 4- to 10-membered heterocyclyl, wherein each alkyl, alkenyl, alkynyl, cycloalkyl, heterocyclyl, aryl, and heteroaryl is optionally substituted with one to three R¹ⁱ;
each R^{1g} is independently —CN, C₁₋₆ alkyl, C₂₋₆ alkynyl, C₃₋₁₀ cycloalkyl, 4- to 10-membered heterocyclyl, C₆₋₁₀ aryl, 5- to 10-membered heteroaryl, —OR^{1h}, —N(R^{1h})(R^{1h}), —C(O)N(R^{1h})(R^{1h}), —N(R^{1h})C(O)—R^{1h}, —N(R^{1h})C(O)O—R^{1h}, —N(R^{1h})C(O)N(R^{1h})(R^{1h}), —OC(O)N(R^{1h})(R^{1h}), —N(R^{1a})S(O)₂(R^{1a}), —S(O)₂R^{1h}, or —S(O)₂N(R^{1a})(R^{1a}), wherein each alkyl, alkynyl, cycloalkyl, heterocyclyl, aryl, and heteroaryl is optionally substituted with one to three R¹ⁱ;
each R^{1h} is independently H, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, or two R^{1h} groups taken together with the nitrogen atom to which they are attached form a 4- to 10-membered heterocyclyl, wherein each alkyl, alkynyl, cycloalkyl, heterocyclyl, aryl and heteroaryl may be further substituted with one to three R¹ⁱ;
each R¹ⁱ is independently halogen, —OH, —NH₂, —CN, C₁₋₆ alkyl, C₁₋₆ haloalkyl, C₁₋₆ hydroxyalkyl, C₃₋₆ cycloalkyl, C₃₋₆ halocycloalkyl, C₁₋₆ alkoxy, C₁₋₆ haloalkoxy, or —O(C₃₋₆ cycloalkyl); L is a bond, —O—, —NR^{La}—, —C(O)NR^{La}, —C(O)—, or C₁₋₃ alkylene, wherein the alkylene is optionally substituted with one to three R^{Lb};
each R^{La} is independently H, C₁₋₆ alkyl, C₁₋₆ haloalkyl, or C₃₋₁₀ cycloalkyl;
each R^{Lb} is independently halogen, —OH, —OCH₃, —NH₂, or —CN;
each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy;
or R^{Y} and R¹ join together to form a C₃₋₆ cycloalkyl or 4- to 6-membered heterocyclyl, wherein each cycloalkyl and heterocyclyl is optionally substituted with one to three halogen;
X is CR³ or N;
R² is —H, halogen, C₁₋₆ alkyl, or C₃₋₆ cycloalkyl, wherein each alkyl and cycloalkyl is optionally substituted with one to three halogen;
R³ is —H, halogen, —CN, C₁₋₆ alkyl, C₁₋₆ haloalkyl, C₁₋₆ hydroxyalkyl, C₁₋₆ alkoxy, C₁₋₆ haloalkoxy, or —O(C₃₋₆ cycloalkyl);
R⁴ is —H, —CN, C₁₋₆ alkyl, C₂₋₆ alkynyl, or C₃₋₁₀ cycloalkyl, wherein each alkyl, alkynyl, and cycloalkyl is optionally substituted with one to three R^{4a1};
each R^{4a1} is independently halogen, —CN, —OR^{4a2}, or —NR^{4a2}R^{4a2}
each R^{4a2} is independently H, C₁₋₆ alkyl, C₁₋₆ haloalkyl, C₃₋₆ cycloalkyl, —C(O)C₁₋₆ alkyl, —C(O)O(C₁₋₆ alkyl), or —C(O)N(R^{4a3})₂;
each R^{4a3} is independently C₁₋₆ alkyl;
ring \textcircled{B} is C₆₋₁₀ aryl or 5- to 10-membered heteroaryl;
each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one to three R^{5b1};
each R^{5a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with one to three R^{5b3};
each R^{5b1} is independently halogen, —CN, C₁₋₆ alkyl, C₂₋₆ alkynyl, C₁₋₆ haloalkyl, C₃₋₁₀ cycloalkyl, 4- to 10-membered heterocyclyl, —OR^{5b2}, or —N(R^{5b2})₂, wherein each alkyl, alkynyl, haloalkyl, cycloalkyl, and heterocycle is optionally substituted with one to three R^{5b3};
each R^{5b2} is independently H, C₁₋₆ alkyl, or C₃₋₁₀ cycloalkyl, wherein each alkyl and cycloalkyl is optionally substituted with one or two oxo and is further optionally substituted with one to three R^{5b3};
each R^{5b3} is independently halogen, —CN, —OH, —NH₂, C₁₋₆ alkyl, C₁₋₆ haloalkyl, C₁₋₆ hydroxyalkyl, C₁₋₆ alkoxy, C₁₋₆ haloalkoxy, C₃₋₆ cycloalkyl, C₃₋₆ halocycloalkyl, or —O(C₃₋₆ cycloalkyl);
ring \textcircled{C} is C₆₋₁₀ aryl, 5- to 10-membered heteroaryl, C₃₋₈ cycloalkyl, or 4- to 10-membered heterocyclyl;
each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further optionally substituted with one to three R^{6b1};
each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl, wherein each alkyl, alkenyl, cycloalkyl, heterocyclyl, aryl, and heteroaryl is optionally substituted with one to three R^{6b1};
each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is optionally substituted with one to three R^{6c1};
each R^{6b2} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, wherein each alkyl, alkenyl, cycloalkyl, heterocyclyl, aryl, and heteroaryl is optionally substituted with one to three R^{6c2}; each R^{6c1} and R^{6c2} is independently oxo, halogen, —NO₂, —N₃, —CN, —OH, R^{6d1}, —O(R^{6d1}), —OC(O)(R^{6d1}), —NH₂, —NH(R^{6d1}), —N(R^{6d1})₂, —C(O)(R^{6d1}), —C(O)O(R^{6d1}), —C(O)NH₂, —C(O)NH(R^{6d1}), —C(O)N(R^{6d1})₂, —NHC(O)(R^{6d1}), —NHC(O)O(R^{6d1}), —NHC(O)NH(R^{6d1}), —SH, —S(R^{6d1}), —NHS(O)(R^{6d1}), —N(R^{6d1})(S(O)(R^{6d1}), —S(O)N(R^{6d1})₂, —S(O)(R^{6d1}), —S(O)(NH)(R^{6d1}), —S(O)₂(R^{6d1}), —S(O)₂NH(R^{6d1}), or —S(O)₂N(R^{6d1})₂;
each R^{6d1} is independently C₁₋₆ alkyl, C₃₋₁₀ cycloalkyl, 4- to 10-membered heterocyclyl, C₆₋₁₀ aryl, and 5- to 10-membered heteroaryl, wherein each alkyl, alkenyl, alkynyl, cycloalkyl, heterocyclyl aryl, and heteroaryl is optionally substituted with one to three R^{6e1};
each R^{6e1} is independently oxo, halogen, —NO₂, —N₃, —CN, —OH, —NH₂, methyl, or halomethyl;
wherein each heterocyclyl and heteroaryl independently has one to four ring heteroatoms each independently selected from N, O, and S; and
each m, n, p and q is independently 0, 1, 2 or 3.

Unless specified otherwise, each 4-membered monocyclic heterocyclyl as used herein has 1 ring heteroatom selected from N, O, and S. Unless specified otherwise, each 5-7 membered monocyclic heterocyclyl as used herein has 1-2 ring heteroatoms independently selected from N, O, and S. Unless specified otherwise, each 6-membered bridged bicyclic heterocyclyl as used herein has 1 ring heteroatom selected from N, O, and S. Unless specified otherwise, each 7-membered bridged bicyclic heterocyclyl as used herein has 1-2 ring heteroatoms independently selected from N, O, and S. Unless specified otherwise, each 5-6 membered monocyclic heteroaryl, 8-10 membered fused bicyclic heterocyclyl, 8-10 membered bridged bicyclic heterocyclyl, 8-10 membered fused bicyclic heteroaryl, and 7-10 membered spirocyclic heterocyclyl as used herein independently have 1-4 ring heteroatoms independently selected from N, O, and S.

In some embodiments, provided herein is a compound of is a compound having Formula (II-A)

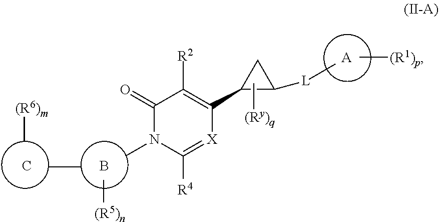

or a pharmaceutically acceptable salt thereof, wherein ring \textcircled{A}, R¹, L, R^{Y}, X, R², R³, R⁴ ring \textcircled{B}, R⁵, ring \textcircled{C}, R⁶, m, n, p, and q are as defined for the compounds of Formula I described herein.

In some embodiments, provided herein is a compound of is a compound having Formula (II-B)

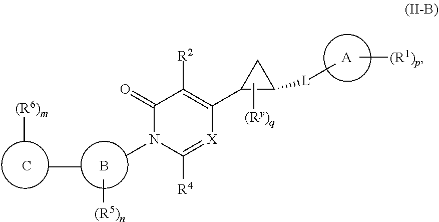

or a pharmaceutically acceptable salt thereof, wherein ring \textcircled{A}, R¹, L, R^{Y}, X, R², R³, R⁴ ring \textcircled{B}, R⁵, ring \textcircled{C}, R⁶, m, n, p, and q are as defined for the compounds of Formula I described herein.

In some embodiments, provided herein is a compound of is a compound having Formula (II)

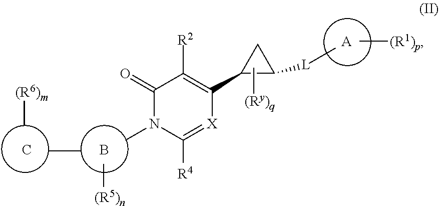

or a pharmaceutically acceptable salt thereof, wherein ring \textcircled{A}, R¹, L, R^{Y}, X, R², R³, R⁴, ring \textcircled{B}, R⁵, ring \textcircled{C}, R⁶, m, n, p, and q are as defined for the compounds of Formula I described herein.

In some embodiments of a compound of Formula I, II, III, IV, V, VI, or VII, or a pharmaceutically acceptable salt thereof, ring \textcircled{A} is C₆₋₁₀ aryl. In some embodiments of a compound of Formula I, II, III, IV, V, VI, or VII, or a pharmaceutically acceptable salt thereof, ring \textcircled{A} is phenyl. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is 5- to 10-membered heteroaryl. In some embodiments of a compound of Formula I, II, III, IV, V, VI, or VII, or a pharmaceutically acceptable salt thereof, ring \textcircled{A} is 5- to 9-membered heteroaryl. In some embodiments of a compound of Formula I, II, III, IV, V, VI, or VII, or a pharmaceutically acceptable salt thereof, ring \textcircled{A} is 5- to 6-membered heteroaryl. In some embodiments of a compound of Formula I, II, III, IV, V, VI, or VII, or a pharmaceutically acceptable salt thereof, ring \textcircled{A} is 5-membered heteroaryl. In some embodiments of a compound of Formula I, II, III, IV, V, VI, or VII, or a pharmaceutically acceptable salt thereof, ring \textcircled{A} is 6-membered heteroaryl. In some embodiments of a compound of Formula I, II, III, IV, V, VI, or VII, or a pharmaceutically acceptable salt thereof, ring \textcircled{A} is 9-membered heteroaryl. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is thiazolyl, isothiazolyl, pyrazolyl, imidazolyl, or pyrrolyl. In some embodiments of a compound of Formula I, II, III, IV, V, VI, or VII, or a pharmaceutically acceptable salt thereof, ring \textcircled{A} is thiazolyl or isothiazolyl. In some embodiments of a compound of Formula I, II, III, IV, V, VI, or VII, or a pharmaceutically acceptable salt thereof, ring \textcircled{A} is pyrazolyl, imidazolyl, or pyrrolyl. In some embodiments of a compound of Formula I, II, III, IV, V, VI, or VII, or a pharmaceutically acceptable salt thereof, ring \textcircled{A} is pyrazolyl. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is pyridinyl, pyrimidinyl, pyrazinyl, or pyridazinyl. In some embodiments of a compound of Formula I, II, III, IV, V, VI, or VII, or a pharmaceutically acceptable salt thereof, ring \textcircled{A} is pyridinyl. In some embodiments, p is 0. In some embodiments, p is 1. In some embodiments, p is 2. In some embodiments, p is 3.

In some embodiments described herein, the various rings (ring A, ring B, and ring C) are shown with the respective substituent (R¹, R⁵, and R⁶, respectively). As can be appreciated by one of skill in the art, reference to ring \textcircled{A}, where the substituent is shown (e.g., ring \textcircled{A} is

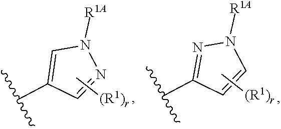

(etc.), refers to

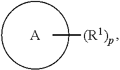

where the groups are defined herein, and the same can apply to ring B and ring C where the substituent is shown.

In some embodiments of a compound of Formula I, II, III, IV, V, VI, or VII, or a pharmaceutically acceptable salt thereof, ring \textcircled{A} is

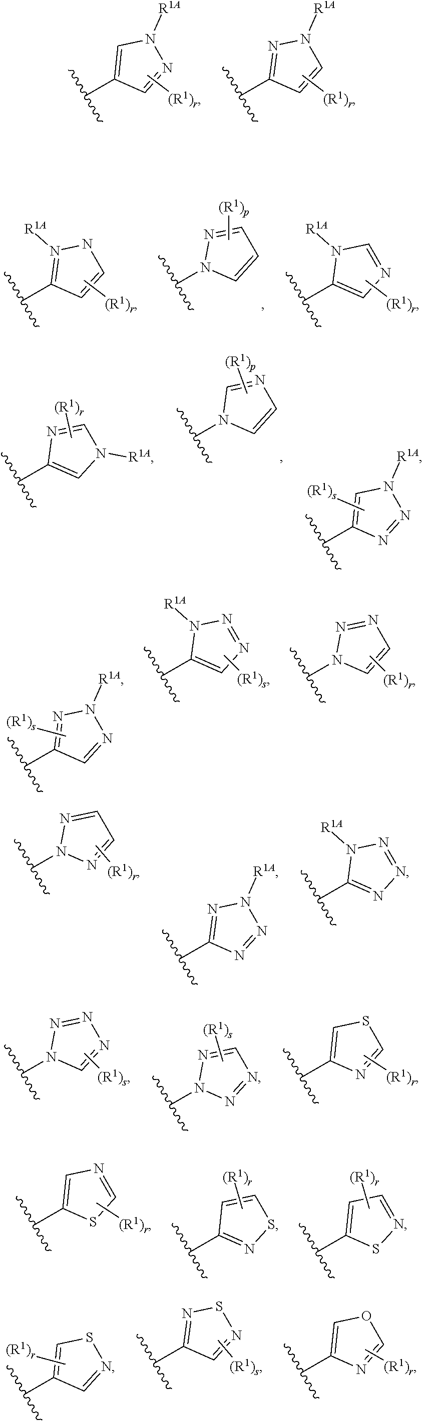

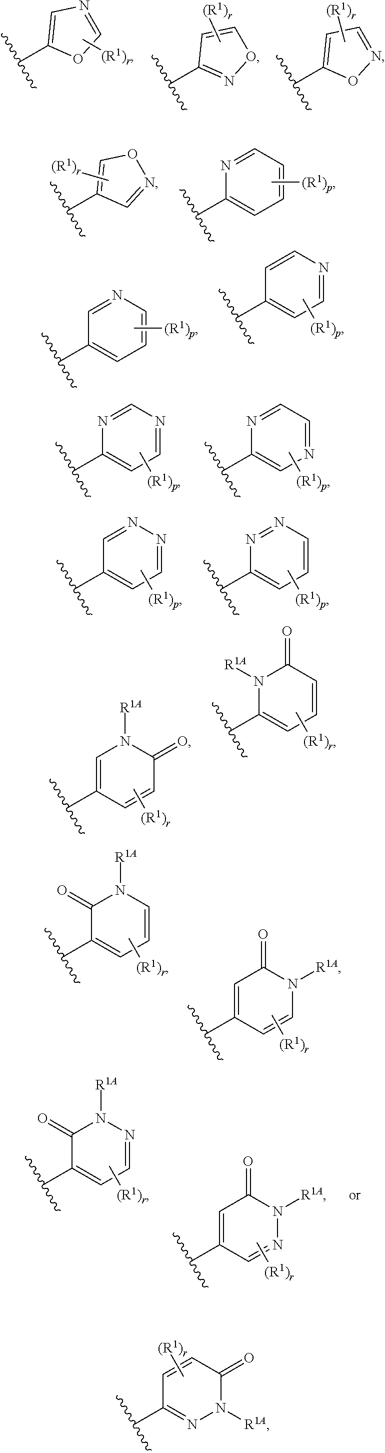

or an N-oxide of any of the foregoing; R^{1A} is H or R^{1A}; r is 0, 1, or 2; and s is 0 or 1. In some embodiments, R^{1A} is H. In some embodiments, R^{1A} is R¹. In some embodiments, r is 0. In some embodiments, r is 1. In some embodiments, r is 2. In some embodiments, s is 0. In some embodiments, s is 1. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

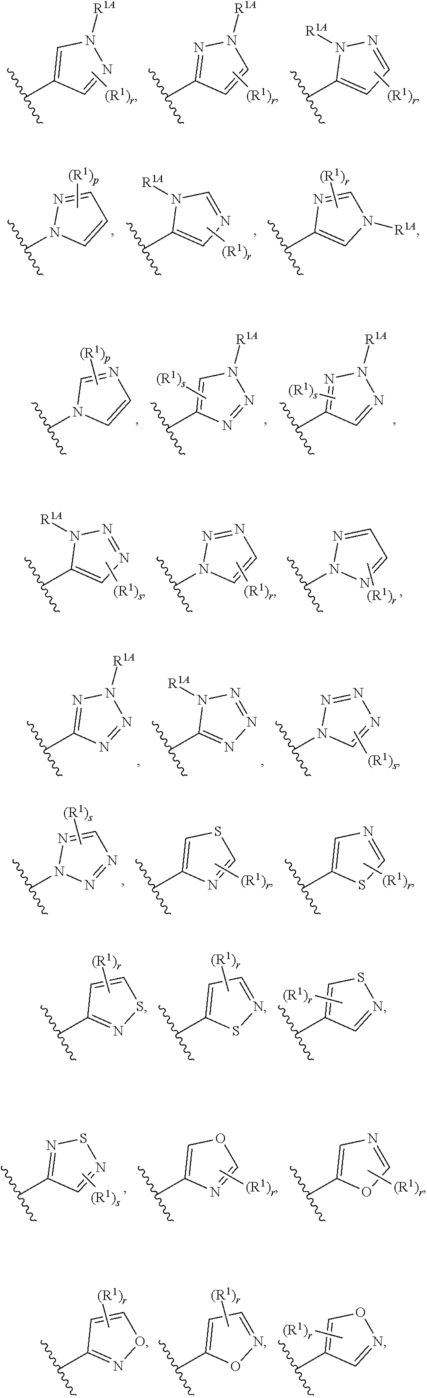

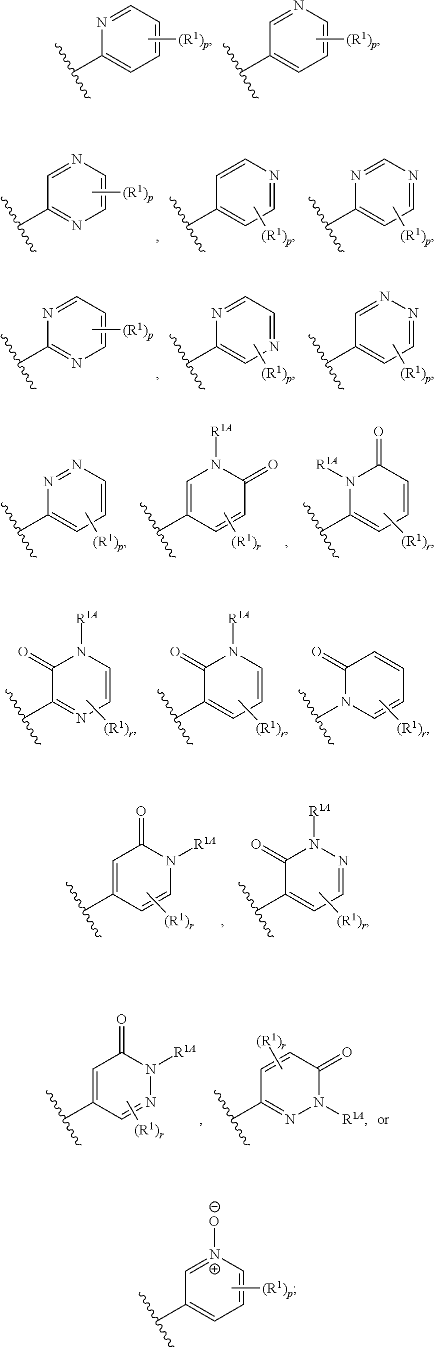

R^{1A} is H or R¹;
r is 0, 1, or 2; and
s is 0 or 1.

In some embodiments of a compound of Formula I, II, III, IV, V, VI, or VII, or a pharmaceutically acceptable salt thereof, ring \textcircled{A} is

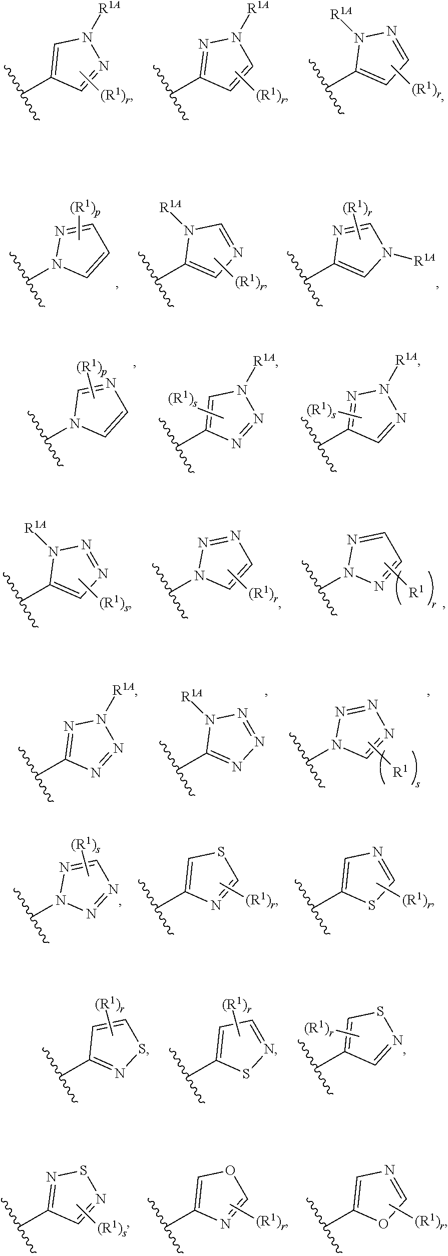

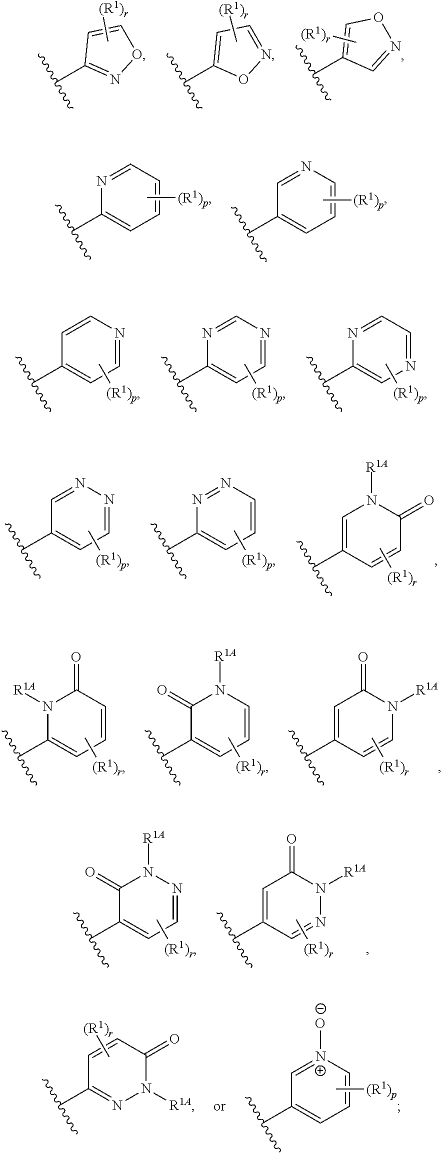

R^{1A} is H or R¹; r is 0, 1, or 2; and s is 0 or 1. In some embodiments, R^{1A} is H. In some embodiments, R^{1A} is R¹. In some embodiments, r is 0. In some embodiments, r is 1. In some embodiments, r is 2. In some embodiments, s is 0. In some embodiments, s is 1. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

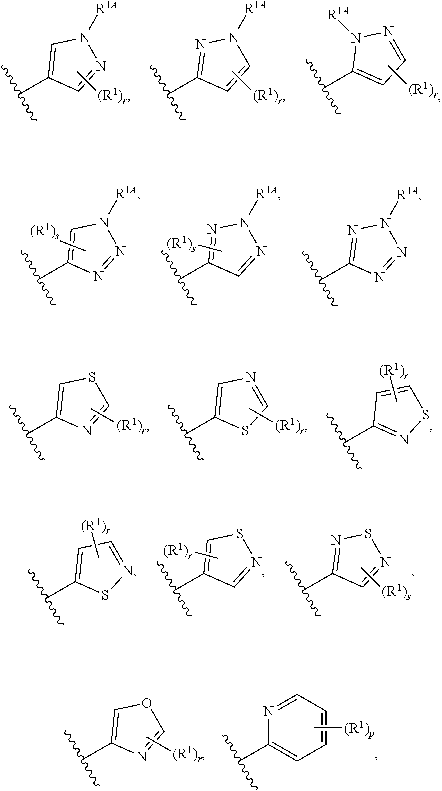

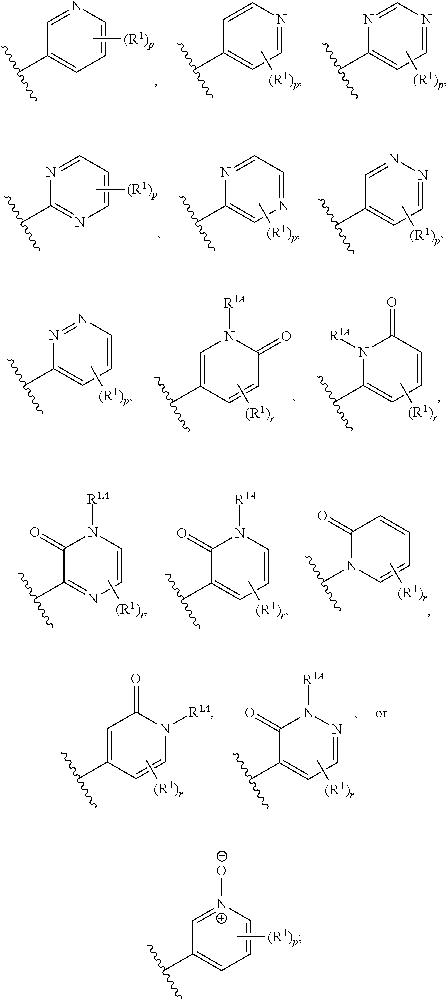

R^{1A} is H or R¹;
r is 0, 1, or 2; and
s is 0 or 1.

In some embodiments of a compound of Formula I, II, III, IV, V, VI, or VII, or a pharmaceutically acceptable salt thereof, ring \textcircled{A} is

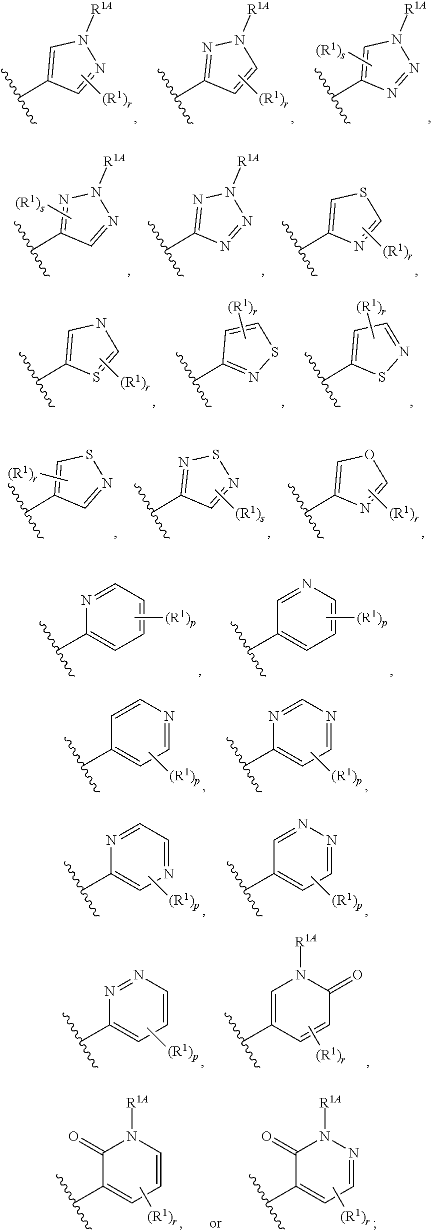

or an N-oxide of any of the foregoing; R^{1A} is H or R¹; r is 0, 1, or 2; and s is 0 or 1. In some embodiments, R^{1A} is H. In some embodiments, R^{1A} is R¹. In some embodiments, r is 0. In some embodiments, r is 1. In some embodiments, r is 2. In some embodiments, s is 0. In some embodiments, s is 1. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

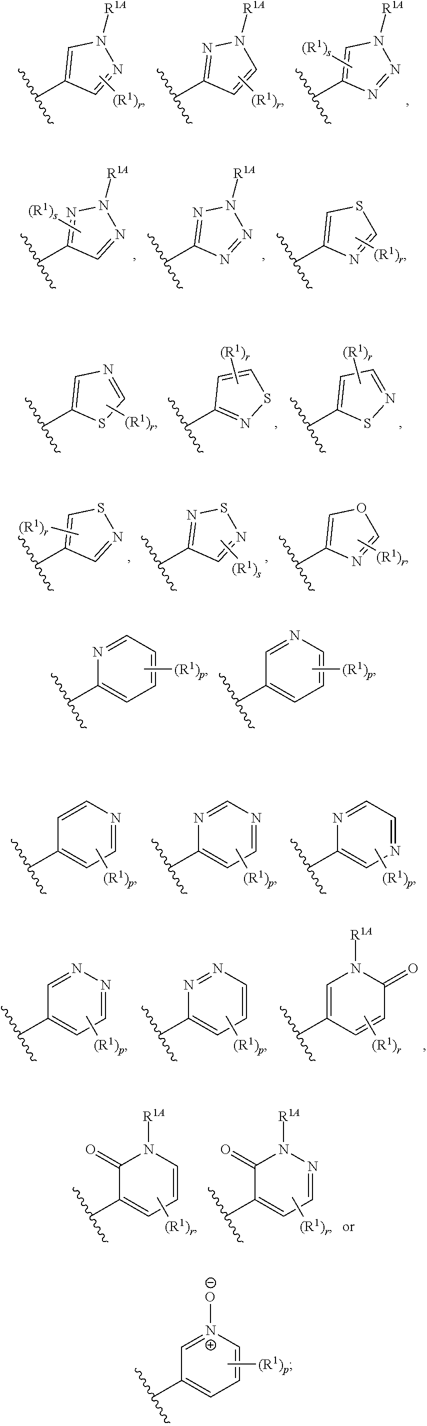

R^{1A} is H or R¹; r is 0, 1, or 2; and s is 0 or 1. In some embodiments, R^{1A} is H. In some embodiments, R^{1A} is R′. In some embodiments, r is 0. In some embodiments, r is 1. In some embodiments, r is 2. In some embodiments, s is 0. In some embodiments, s is 1. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

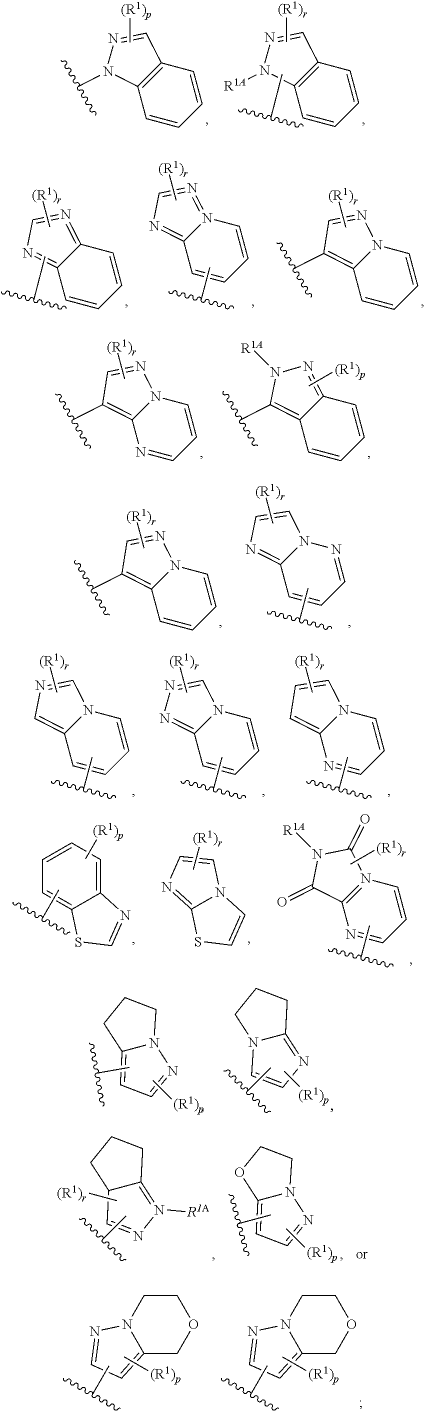

or an N-oxide of any of the foregoing; R^{1A} is H or R¹; and r is 0, 1, or 2.

In some embodiments of a compound of Formula I, II, III, IV, V, VI, or VII, or a pharmaceutically acceptable salt thereof, ring \textcircled{A} is

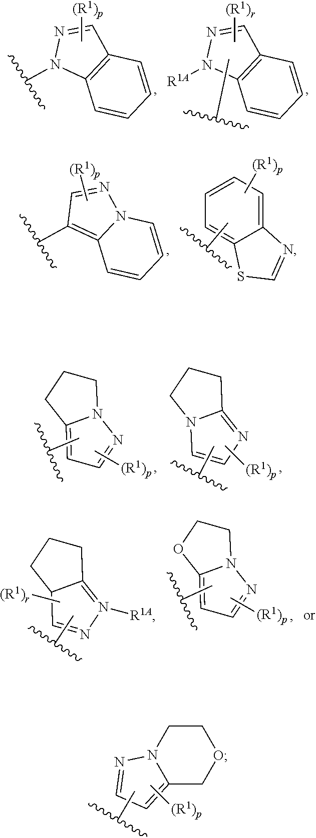

or an N-oxide of any of the foregoing; R^{1A} is H or R¹; and r is 0, 1, or 2. In some embodiments, R^{1A} is H. In some embodiments, R^{1A} is R¹. In some embodiments, r is 0. In some embodiments, r is 1. In some embodiments, r is 2. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is,

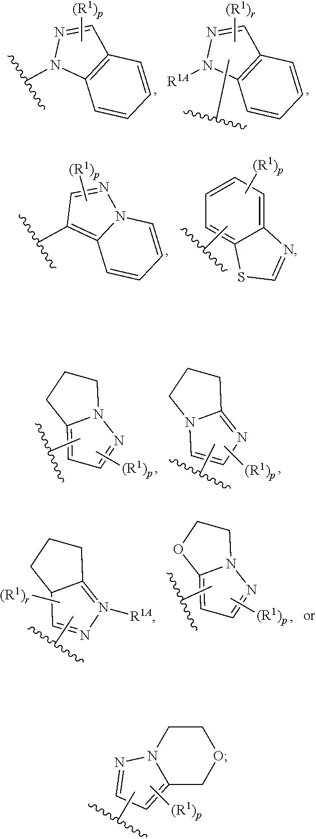

R^{1A} is H or R¹; and r is 0, 1, or 2. In some embodiments, R^{1A} is H. In some embodiments, R^{1A} is R¹. In some embodiments, r is 0. In some embodiments, r is 1. In some embodiments, r is 2. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

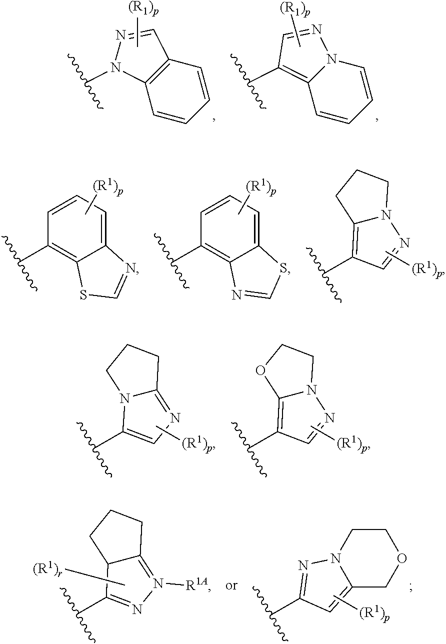

or an N-oxide of any of the foregoing; R^{1A} is H or R¹; and r is 0, 1, or 2. In some embodiments, R^{1A} is H. In some embodiments, R^{1A} is R¹. In some embodiments, r is 0. In some embodiments, r is 1. In some embodiments, r is 2. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

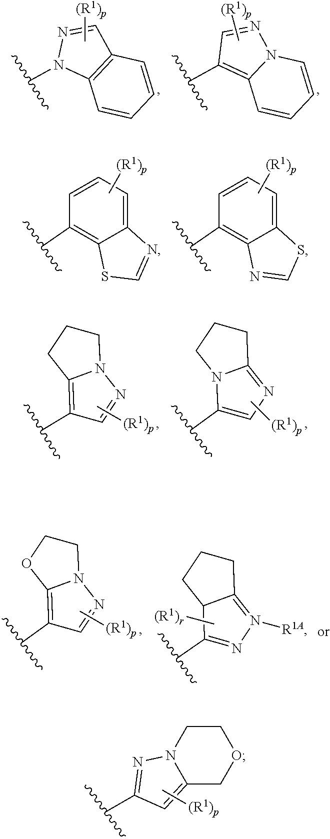

R^{1A} is H or R¹; and r is 0, 1, or 2. In some embodiments, R^{1A} is H. In some embodiments, R^{1A} is R¹. In some embodiments, r is 0. In some embodiments, r is 1. In some embodiments, r is 2. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

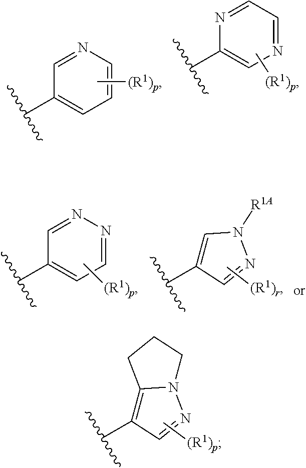

or an N-oxide of any of the foregoing; R^{1A} is H or R¹; and r is 0, 1, or 2. In some embodiments, R^{1A} is H. In some embodiments, R^{1A} is R¹. In some embodiments, r is 0. In some embodiments, r is 1. In some embodiments, r is 2. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

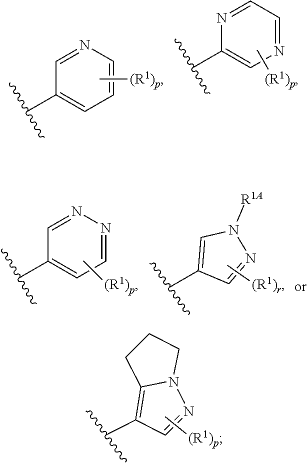

R^{1A} is H or R¹; and r is 0, 1, or 2. In some embodiments, R^{1A} is H. In some embodiments, R^{1A} is R¹. In some embodiments, r is 0. In some embodiments, r is 1. In some embodiments, r is 2. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

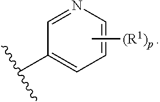

In some embodiments of a compound of Formula I, II, III, IV, V, VI, or VII, or a pharmaceutically acceptable salt thereof, ring \textcircled{A} is

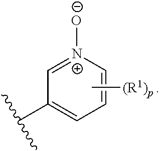

In some embodiments of a compound of Formula I, II, III, IV, V, VI, or VII, or a pharmaceutically acceptable salt thereof, ring \textcircled{A} is

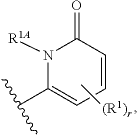

wherein R^{1A} and r are as defined herein. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

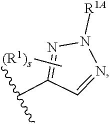

R^{1A} is H or R¹; and
r is 0 or 1.

In some embodiments of a compound of Formula I, II, III, IV, V, VI, or VII, or a pharmaceutically acceptable salt thereof, ring \textcircled{A} is

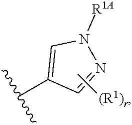

R^{1A} is H or R¹; and
r is 0, 1, or 2.

In some embodiments of a compound of Formula I, II, III, IV, V, VI, or VII, or a pharmaceutically acceptable salt thereof, ring \textcircled{A} is

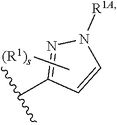

R^{1A} is H or R¹; and
r is 0, 1, or 2.

In some embodiments, R¹ or R^{1A} or both R¹ and R^{1A} are —CD₃.

In some embodiments, each R¹ is independently Cl, or F, and R^{1A} is —CD₃.

In some embodiments of a compound of Formula I, II, III, IV, V, VI, or VII, or a pharmaceutically acceptable salt thereof, ring \textcircled{A} is C₃₋₁₀ cycloalkyl. In some embodiments of a compound of Formula I, II, III, IV, V, VI, or VII, or a pharmaceutically acceptable salt thereof, ring \textcircled{A} is C₃₋₆ cycloalkyl. In some embodiments of a compound of Formula I, II, III, IV, V, VI, or VII, or a pharmaceutically acceptable salt thereof, ring \textcircled{A} is cyclopropyl. In some embodiments of a compound of Formula I, II, III, IV, V, VI, or VII, or a pharmaceutically acceptable salt thereof, ring \textcircled{A} is cyclobutyl. In some embodiments of a compound of Formula I, II, III, IV, V, VI, or VII, or a pharmaceutically acceptable salt thereof, ring \textcircled{A} is cyclopentyl. In some embodiments of a compound of Formula I, II, III, IV, V, VI, or VII, or a pharmaceutically acceptable salt thereof, ring \textcircled{A} is cyclohexyl. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is 4- to 10-membered heterocyclyl. In some embodiments of a compound of Formula I, II, III, IV, V, VI, or VII, or a pharmaceutically acceptable salt thereof, ring \textcircled{A} is 4- to 6-membered heterocyclyl. In some embodiments of a compound of Formula I, II, III, IV, V, VI, or VII, or a pharmaceutically acceptable salt thereof, ring is 5- to 6-membered heterocyclyl. In some embodiments of a compound of Formula I, II, III, IV, V, VI, or VII, or a pharmaceutically acceptable salt thereof, ring \textcircled{A} is 5-membered heterocyclyl. In some embodiments of a compound of Formula I, II, III, IV, V, VI, or VII, or a pharmaceutically acceptable salt thereof, ring \textcircled{A} is 6-membered heterocyclyl. In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, ring \textcircled{A} is

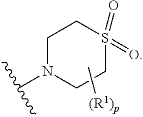

In some embodiments, p is 0. In some embodiments, p is 1. In some embodiments, p is 2. In some embodiments, p is 3.

In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further optionally substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further optionally substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1I}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further optionally substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R¹, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, —S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to three halogen and is further optionally substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to three halogen and is further optionally substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to three halogen and is further optionally substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a} S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to three halogen and is further optionally substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to three halogen and is further substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to three halogen and is further substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to three halogen and is further substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to three halogen and is further substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(Ra)(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a} S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is optionally substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, wherein each alkyl, alkynyl, cycloalkyl, heterocyclyl, aryl, and heteroaryl is substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a}))C(O)R^{1a}, or —P(O)(R^{1a})₂, wherein each alkyl, alkynyl, cycloalkyl, heterocyclyl, aryl, and heteroaryl is substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further optionally substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further optionally substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(Ra)(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further optionally substituted with one R^{1g}.

In some embodiments of a compound of Formula I, II, III, IV, V, VI, or VII, or a pharmaceutically acceptable salt thereof, each R¹ is independently halogen, —CD₃, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g}.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is optionally substituted with one to three halogen and is further optionally substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is optionally substituted with one to three halogen and is further optionally substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is optionally substituted with one to three halogen and is further optionally substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), wherein each alkyl, alkynyl, cycloalkyl, heterocyclyl, aryl, and heteroaryl is optionally substituted with one to three halogen and is further optionally substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is optionally substituted with one to three halogen and is further substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is optionally substituted with one to three halogen and is further substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is optionally substituted with one to three halogen and is further substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is optionally substituted with one to three halogen and is further substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is optionally substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), wherein each alkyl, alkynyl, cycloalkyl, heterocyclyl, aryl, and heteroaryl is optionally substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is optionally substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR¹, —C(O)OR¹, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), wherein each alkyl, alkynyl, cycloalkyl, heterocyclyl, aryl, and heteroaryl is optionally substituted with one R^{1g}.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is substituted with one to three R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), wherein each alkyl, alkynyl, cycloalkyl, heterocyclyl, aryl, and heteroaryl is substituted with one to two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}) wherein each alkyl, alkynyl, cycloalkyl, heterocyclyl, aryl, and heteroaryl is substituted with two R^{1g}. In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), wherein each alkyl, alkynyl, cycloalkyl, heterocyclyl, aryl, and heteroaryl is substituted with one R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to three halogen and is further optionally substituted with one to three R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to three halogen and is further optionally substituted with one to two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to three halogen and is further optionally substituted with two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to three halogen and is further optionally substituted with one R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to three halogen and is further substituted with one to three R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to three halogen and is further substituted with one to two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to three halogen and is further substituted with two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to three halogen and is further substituted with one R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to three R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one to two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is optionally substituted with one R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is substituted with one to three R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is substituted with one to two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein the heterocyclyl is substituted with two R^{1g}. In some embodiments of a compound of Formula I, II, III, IV, V, VI, or VII, or a pharmaceutically acceptable salt thereof, two R¹ groups taken together with two adjacent atoms of ring 71 to which they are attached form a 4- to 6-membered heterocyclyl, wherein the heterocyclyl is substituted with one R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to three halogen and is further optionally substituted with one to three R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to three halogen and is further optionally substituted with one to two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to three halogen and is further optionally substituted with two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to three halogen and is further optionally substituted with one R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to three halogen and is further substituted with one to three R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to three halogen and is further substituted with one to two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to three halogen and is further substituted with two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to three halogen and is further substituted with one R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to three R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one to two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is optionally substituted with one R^{1g}.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is substituted with one to three R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is substituted with one to two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is substituted with two R^{1g}. In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein the heterocyclyl is substituted with one R^{1g}.

In some embodiments of a compound of Formula I, II, III, IV, V, VI, or VII, or a pharmaceutically acceptable salt thereof R^{1g} is —CN, C₁₋₆ alkyl, C₂₋₆ alkynyl, C₃₋₁₀ cycloalkyl, 4- to 10-membered heterocyclyl, C₆₋₁₀ aryl, 5- to 10-membered heteroaryl, —OR^{1h}, —N(R^{1h})(R^{1h}), —C(O)N(R^{1h})(R^{1h}), —N(R^{1h})C(O)—R^{1h}, —N(R^{1h})C(O)O—R^{1h}, —N(R^{1h})C(O)N(R^{1h})(R^{1h}), —OC(O)N(R^{1h})(R^{1h}), —N(R^{1a})S(O)₂(R^{1a}), —S(O)₂R^{1h}, or —S(O)₂N(R^{1a})(R^{1a}), wherein each alkyl, alkynyl, cycloalkyl, heterocyclyl, aryl, and heteroaryl is optionally substituted with one to three R¹ⁱ.

In some embodiments of a compound of Formula I, II, III, IV, V, VI, or VII, or a pharmaceutically acceptable salt thereof R^{1g} is —CN, C₁₋₆ alkyl, C₂₋₆ alkynyl, C₃₋₁₀ cycloalkyl, 4- to 10-membered heterocyclyl, C₆₋₁₀ aryl, 5- to 10-membered heteroaryl, —OR^{1h}, —N(R^{1h})(R^{1h}), —C(O)N(R^{1h})(R^{1h}), —N(R^{1h})C(O)—R^{1h}, —N(R^{1h})C(O)O—R^{1h}, —N(R^{1h})C(O)N(R^{1h})(R^{1h}), —OC(O)N(R^{1h})(R^{1h}), —N(R^{1a})S(O)₂(R^{1a}), —S(O)₂R^{1h}, or —S(O)₂N(R^{1a})(R^{1a}), wherein each alkyl, alkynyl, cycloalkyl, heterocyclyl, aryl, and heteroaryl is substituted with one to three R¹ⁱ.

In some embodiments of a compound of Formula I, II, III, IV, V, VI, or VII, or a pharmaceutically acceptable salt thereof R^{1g} is —CN, C₁₋₆ alkyl, C₂₋₆ alkynyl, C₃₋₁₀ cycloalkyl, 4- to 10-membered heterocyclyl, C₆₋₁₀ aryl, 5- to 10-membered heteroaryl, —OR^{1h}, —N(R^{1h})(R^{1h}), —C(O)N(R^{1h})(R^{1h}), —N(R^{1h})C(O)—R^{1h}, —N(R^{1h})C(O)O—R^{1h}, —N(R^{1h})C(O)N(R^{1h})(R^{1h}), —OC(O)N(R^{1h})(R^{1h}), —N(R^{1a})S(O)₂(R^{1a}), —S(O)₂R^{1h} or —S(O)₂N(R^{1a})(R^{1a}).

In some embodiments of a compound of Formula I, II, III, IV, V, VI, or VII, or a pharmaceutically acceptable salt thereof R^{1g} is C₁₋₆ alkyl, wherein each alkyl is optionally substituted with one to three halogen. In some embodiments of a compound of Formula I, II, III, IV, V, VI, or VII, or a pharmaceutically acceptable salt thereof R^{1g} is C₁₋₃ alkyl, wherein each alkyl is optionally substituted with one to three halogen. In some embodiments of a compound of Formula I, II, III, IV, V, VI, or VII, or a pharmaceutically acceptable salt thereof R^{1g} is C₁₋₂ alkyl, wherein each alkyl is optionally substituted with one to three halogen. In some embodiments of a compound of Formula I, II, III, IV, V, VI, or VII, or a pharmaceutically acceptable salt thereof R^{1g} is methyl, wherein each methyl is optionally substituted with one to three halogen. In some embodiments of a compound of Formula I, II, III, IV, V, VI, or VII, or a pharmaceutically acceptable salt thereof the halogen is fluoro. In some embodiments of a compound of Formula I, II, III, IV, V, VI, or VII, or a pharmaceutically acceptable salt thereof the halogen is chloro.

In some embodiments of a compound of Formula I, II, III, IV, V, VI, or VII, or a pharmaceutically acceptable salt thereof R^{1g} is C₁₋₆ alkyl. In some embodiments of a compound of Formula I, II, III, IV, V, VI, or VII, or a pharmaceutically acceptable salt thereof R^{1g} is C₁₋₃ alkyl. In some embodiments of a compound of Formula I, II, III, IV, V, VI, or VII, or a pharmaceutically acceptable salt thereof R^{1g} is C₁₋₂ alkyl. In some embodiments of a compound of Formula I, II, III, IV, V, VI, or VII, or a pharmaceutically acceptable salt thereof R^{1g} is methyl.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, cycloalkyl, and heterocyclyl is optionally substituted with one to three halogen.

In some embodiments of a compound of Formula I, II, III, IV, V, VI, or VII, or a pharmaceutically acceptable salt thereof no group is substituted with R¹⁸.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, cycloalkyl, and heterocyclyl is substituted with one to three halogen.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —N═S(O)(R^{1a})(R^{1a}), or two R¹ groups taken together with two adjacent atoms of ring \textcircled{A} to which they are attached form a 4- to 10-membered heterocyclyl.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —N═S(O)(R^{1a})(R^{1a}) wherein each alkyl, cycloalkyl, and heterocyclyl is optionally substituted with one to three halogen.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —N═S(O)(R^{1a})(R^{1a}), wherein each alkyl, cycloalkyl, and heterocyclyl is substituted with one to three halogen.

In some embodiments of a compound of Formula I, II, III, IV, V, VI, or VII, or a pharmaceutically acceptable salt thereof, each R¹ is independently halogen, C₁₋₆ alkyl, C₃₋₁₀ cycloalkyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —N═S(O)(R^{1a})(R a).

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 10-membered heterocyclyl, wherein each alkyl, cycloalkyl, and heterocyclyl is optionally substituted with one to three halogen.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 10-membered heterocyclyl, wherein each alkyl, cycloalkyl, and heterocyclyl is substituted with one to three halogen.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 10-membered heterocyclyl.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein each alkyl, cycloalkyl, and heterocyclyl is optionally substituted with one to three halogen.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 4- to 6-membered heterocyclyl, wherein each alkyl, cycloalkyl, and heterocyclyl is substituted with one to three halogen.

In some embodiments of a compound of Formula I, II, III, IV, V, VI, or VII, or a pharmaceutically acceptable salt thereof, two R¹ groups taken together with two adjacent atoms of ring A to which they are attached form a 4- to 6-membered heterocyclyl.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein each alkyl, cycloalkyl, and heterocyclyl is optionally substituted with one to three halogen.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl, wherein each alkyl, cycloalkyl, and heterocyclyl is substituted with one to three halogen.

In some embodiments of a compound of Formula I, II, III, IV, V, VI, or VII, or a pharmaceutically acceptable salt thereof, two R¹ groups taken together with two adjacent atoms of ring \textcircled{A} to which they are attached form a 5- to 6-membered heterocyclyl.

In some embodiments of a compound of Formula I, II, III, IV, V, VI, or VII, or a pharmaceutically acceptable salt thereof, each R¹ is independently halogen, C₁₋₆ alkyl, or —C(O)N(R^{1a})(R^{1a}), wherein each alkyl is optionally substituted with one to three halogen.

In some embodiments of a compound of Formula I, II, III, IV, V, VI, or VII, or a pharmaceutically acceptable salt thereof, each R¹ is independently halogen, C₁₋₆ alkyl, or —C(O)N(R^{1a})(R^{1a}), wherein each alkyl is substituted with one to three halogen.

In some embodiments of a compound of Formula I, II, III, IV, V, VI, or VII, or a pharmaceutically acceptable salt thereof, each R¹ is independently halogen, C₁₋₆ alkyl, or —C(O)N(R^{1a})(R^{1a}).

In some embodiments of a compound of Formula I, II, III, IV, V, VI, or VII, or a pharmaceutically acceptable salt thereof, each R¹ is independently halogen. In some embodiments of a compound of Formula I, II, III, IV, V, VI, or VII, or a pharmaceutically acceptable salt thereof, at least one R¹ is halogen.

In some embodiments of a compound of Formula I, II, III, IV, V, VI, or VII, or a pharmaceutically acceptable salt thereof, each R¹ is independently chloro. In some embodiments of a compound of Formula I, II, III, IV, V, VI, or VII, or a pharmaceutically acceptable salt thereof, at least one R¹ is chloro.

In some embodiments of a compound of Formula I, II, III, IV, V, VI, or VII, or a pharmaceutically acceptable salt thereof, each R¹ is independently fluoro. In some embodiments of a compound of Formula I, II, III, IV, V, VI, or VII, or a pharmaceutically acceptable salt thereof, at least one R¹ is fluoro.

In some embodiments of a compound of Formula I, II, III, IV, V, VI, or VII, or a pharmaceutically acceptable salt thereof, each R¹ is independently C₁₋₆ alkyl, wherein each alkyl optionally is substituted with one to three halogen. In some embodiments of a compound of Formula I, II, III, IV, V, VI, or VII, or a pharmaceutically acceptable salt thereof, each R¹ is independently C₁₋₃ alkyl, wherein each alkyl optionally is substituted with one to three halogen. In some embodiments of a compound of Formula I, II, III, IV, V, VI, or VII, or a pharmaceutically acceptable salt thereof, each R¹ is independently methyl, wherein each alkyl optionally is substituted with one to three halogen.

In some embodiments of a compound of Formula I, II, III, IV, V, VI, or VII, or a pharmaceutically acceptable salt thereof, at least one R¹ is C₁₋₆ alkyl, wherein each alkyl optionally is substituted with one to three halogen. In some embodiments of a compound of Formula I, II, III, IV, V, VI, or VII, or a pharmaceutically acceptable salt thereof, at least one R¹ is C₁₋₃ alkyl, wherein each alkyl optionally is substituted with one to three halogen. In some embodiments of a compound of Formula I, II, III, IV, V, VI, or VII, or a pharmaceutically acceptable salt thereof, at least one R¹ is methyl, wherein each alkyl optionally is substituted with one to three halogen.

In some embodiments of a compound of Formula I, II, III, IV, V, VI, or VII, or a pharmaceutically acceptable salt thereof, each R¹ is independently C₁₋₆ alkyl, wherein each alkyl is substituted with one to three halogen. In some embodiments of a compound of Formula I, II, III, IV, V, VI, or VII, or a pharmaceutically acceptable salt thereof, each R¹ is independently C₁₋₃ alkyl, wherein each alkyl is substituted with one to three halogen. In some embodiments of a compound of Formula I, II, III, IV, V, VI, or VII, or a pharmaceutically acceptable salt thereof, each R¹ is independently methyl, wherein each alkyl is substituted with one to three halogen.

In some embodiments of a compound of Formula I, II, III, IV, V, VI, or VII, or a pharmaceutically acceptable salt thereof, at least one R¹ is C₁₋₆ alkyl, wherein each alkyl is substituted with one to three halogen. In some embodiments of a compound of Formula I, II, III, IV, V, VI, or VII, or a pharmaceutically acceptable salt thereof, at least one R¹ is C₁₋₃ alkyl, wherein each alkyl is substituted with one to three halogen. In some embodiments of a compound of Formula I, II, III, IV, V, VI, or VII, or a pharmaceutically acceptable salt thereof, at least one R¹ is methyl, wherein each alkyl is substituted with one to three halogen.

In some embodiments of a compound of Formula I, II, III, IV, V, VI, or VII, or a pharmaceutically acceptable salt thereof, each R¹ is independently halogen or C₁₋₆ alkyl. In some embodiments of a compound of Formula I, II, III, IV, V, VI, or VII, or a pharmaceutically acceptable salt thereof, each R¹ is independently halogen or C₁₋₃ alkyl.

In some embodiments of a compound of Formula I, II, III, IV, V, VI, or VII, or a pharmaceutically acceptable salt thereof, each R¹ is independently C₁₋₆ alkyl. In some embodiments of a compound of Formula I, II, III, IV, V, VI, or VII, or a pharmaceutically acceptable salt thereof, each R¹ is independently C₁₋₃ alkyl. In some embodiments of a compound of Formula I, II, III, IV, V, VI, or VII, or a pharmaceutically acceptable salt thereof, each R¹ is independently methyl.

In some embodiments of a compound of Formula I, II, III, IV, V, VI, or VII, or a pharmaceutically acceptable salt thereof, at least one R¹ is C₁₋₆ alkyl. In some embodiments of a compound of Formula I, II, III, IV, V, VI, or VII, or a pharmaceutically acceptable salt thereof, at least one R¹ is C₁₋₃ alkyl. In some embodiments of a compound of Formula I, II, III, IV, V, VI, or VII, or a pharmaceutically acceptable salt thereof, at least one R¹ is methyl.

In some embodiments of a compound of Formula I, II, III, IV, V, VI, or VII, or a pharmaceutically acceptable salt thereof, each R¹ is independently halogen. In some embodiments of a compound of Formula I, II, III, IV, V, VI, or VII, or a pharmaceutically acceptable salt thereof, each R¹ is independently chloro or fluoro. In some embodiments of a compound of Formula I, II, III, IV, V, VI, or VII, or a pharmaceutically acceptable salt thereof, at least one R¹ is halogen. In some embodiments of a compound of Formula I, II, III, IV, V, VI, or VII, or a pharmaceutically acceptable salt thereof, at least one R¹ is fluoro. In some embodiments of a compound of Formula I, II, III, IV, V, VI, or VII, or a pharmaceutically acceptable salt thereof, at least one R¹ is chloro.

In some embodiments of a compound of Formula I, II, III, IV, V, VI, or VII, or a pharmaceutically acceptable salt thereof, each R^{1a} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, two R^{1a} groups taken together with the nitrogen atom to which they are attached form a 4- to 10-membered heterocyclyl, wherein each alkyl, alkenyl, alkynyl, cycloalkyl, heterocyclyl, aryl, and heteroaryl is optionally substituted with one to three R¹ⁱ. In some embodiments of a compound of Formula I, II, III, IV, V, VI, or VII, or a pharmaceutically acceptable salt thereof, each R^{1a} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, two R^{1a} groups taken together with the nitrogen atom to which they are attached form a 4- to 10-membered heterocyclyl, wherein each alkyl, alkenyl, alkynyl, cycloalkyl, heterocyclyl, aryl, and heteroaryl is substituted with one to three R¹ⁱ. In some embodiments of a compound of Formula I, II, III, IV, V, VI, or VII, or a pharmaceutically acceptable salt thereof, each R^{1a} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, two R^{1a} groups taken together with the nitrogen atom to which they are attached form a 4- to 10-membered heterocyclyl.

In some embodiments of a compound of Formula I, II, III, IV, V, VI, or VII, or a pharmaceutically acceptable salt thereof, each R^{1a} is independently H, C₁₋₆ alkyl, or C₃₋₁₀ cycloalkyl wherein each alkyl and cycloalkyl is optionally substituted with one to three R¹ⁱ. In some embodiments of a compound of Formula I, II, III, IV, V, VI, or VII, or a pharmaceutically acceptable salt thereof, each R^{1a} is independently H, C₁₋₆ alkyl, or C₃₋₁₀ cycloalkyl wherein each alkyl and cycloalkyl is substituted with one to three R¹ⁱ. In some embodiments of a compound of Formula I, II, III, IV, V, VI, or VII, or a pharmaceutically acceptable salt thereof, each R^{1a} is independently H, C₁₋₆ alkyl, or C₃₋₁₀ cycloalkyl.

In some embodiments of a compound of Formula I, II, III, IV, V, VI, or VII, or a pharmaceutically acceptable salt thereof, each R^{1a} is independently H, C₁₋₃ alkyl, or C₃₋₆ cycloalkyl wherein each alkyl and cycloalkyl is optionally substituted with one to three R¹. In some embodiments of a compound of Formula I, II, III, IV, V, VI, or VII, or a pharmaceutically acceptable salt thereof, each R^{1a} is independently H, C₁₋₃ alkyl, or C₃₋₆ cycloalkyl wherein each alkyl and cycloalkyl is substituted with one to three R¹ⁱ. In some embodiments of a compound of Formula I, II, III, IV, V, VI, or VII, or a pharmaceutically acceptable salt thereof, each R^{1a} is independently H, C₁₋₃ alkyl, or C₃₋₆ cycloalkyl.

In some embodiments of a compound of Formula I, II, III, IV, V, VI, or VII, or a pharmaceutically acceptable salt thereof, each R¹ⁱ is independently halogen, —OH, —NH₂, —CN, C₁₋₆ alkyl, C₁₋₆ haloalkyl, C₁₋₆ hydroxyalkyl, C₃₋₆ cycloalkyl, C₃₋₆ halocycloalkyl, C₁₋₆ alkoxy, C₁₋₆ haloalkoxy, or —O(C₃₋₆ cycloalkyl).

In some embodiments of a compound of Formula I, II, III, IV, V, VI, or VII, or a pharmaceutically acceptable salt thereof, each R¹ⁱ is halogen. In some embodiments of a compound of Formula I, II, III, IV, V, VI, or VII, or a pharmaceutically acceptable salt thereof, each R¹ⁱ is independently chloro or fluoro. In some embodiments of a compound of Formula I, II, III, IV, V, VI, or VII, or a pharmaceutically acceptable salt thereof, R¹ⁱ is chloro. In some embodiments of a compound of Formula I, II, III, IV, V, VI, or VII, or a pharmaceutically acceptable salt thereof, R¹ⁱ is fluoro.

In some embodiments of a compound of Formula I, II, III, IV, V, VI, or VII, or a pharmaceutically acceptable salt thereof, each R^{1h} is independently H, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, or two R^{1h} groups taken together with the nitrogen atom to which they are attached form a 4- to 10-membered heterocyclyl, wherein each alkyl, alkynyl, cycloalkyl, heterocyclyl, aryl and heteroaryl may be further substituted with one to three R¹ⁱ.

In some embodiments of a compound of Formula I, II, III, IV, V, VI, or VII, or a pharmaceutically acceptable salt thereof, L is a bond, —O—, —NR^{La}—, —C(O)NR^{La}—, —C(O), or C₁₋₃ alkylene, wherein the alkylene is optionally substituted with one to three R^{Lb}. In some embodiments of a compound of Formula I, II, III, IV, V, VI, or VII, or a pharmaceutically acceptable salt thereof, L is a bond, —O—, —NR^{La}—, —C(O)NR^{La}—, —C(O)—, or C₁₋₃ alkylene, wherein the alkylene is optionally substituted with one to two R^{Lb}. In some embodiments of a compound of Formula I, II, III, IV, V, VI, or VII, or a pharmaceutically acceptable salt thereof, L is a bond, —O—, —NR^{La}—, —C(O)NR^{La}—, —C(O), or C₁₋₃ alkylene, wherein the alkylene is optionally substituted with two R^{Lb}. In some embodiments of a compound of Formula I, II, III, IV, V, VI, or VII, or a pharmaceutically acceptable salt thereof, L is a bond, —O—, —NR^{La}—, —C(O)NR^{La}—, —C(O)—, or C₁₋₃ alkylene, wherein the alkylene is optionally substituted with one R^{Lb}.

In some embodiments of a compound of Formula I, II, III, IV, V, VI, or VII, or a pharmaceutically acceptable salt thereof, L is a bond, —O—, —NR^{La}—, —C(O)NR^{La}—, —C(O), or C₁₋₃ alkylene, wherein the alkylene is substituted with one to three R^{Lb}. In some embodiments of a compound of Formula I, II, III, IV, V, VI, or VII, or a pharmaceutically acceptable salt thereof, L is a bond, —O—, —NR^{La}—, —C(O)NR^{La}, —C(O)—, or C₁₋₃ alkylene, wherein the alkylene is substituted with one to two R^{Lb} in some embodiments of a compound of Formula I, II, III, IV, V, VI, or VII, or a pharmaceutically acceptable salt thereof, L is a bond, —O—, —NR^{La}—, —C(O)NR^{La}, —C(O)—, or C₁₋₃ alkylene, wherein the alkylene is substituted with two R^{Lb}. In some embodiments of a compound of Formula I, II, III, IV, V, VI, or VII, or a pharmaceutically acceptable salt thereof, L is a bond, —O—, —NR^{La}—, —C(O)NR^{La}—, —C(O)—, or C₁₋₃ alkylene, wherein the alkylene is substituted with one R^{Lb}.

In some embodiments of a compound of Formula I, II, III, IV, V, VI, or VII, or a pharmaceutically acceptable salt thereof, L is a bond, —C(O)NR^{La}—, —C(O)—, or C₁₋₃ alkylene, wherein the alkylene is optionally substituted with one to three R^{Lb}. In some embodiments of a compound of Formula I, II, III, IV, V, VI, or VII, or a pharmaceutically acceptable salt thereof, L is a bond, —C(O)NR^{La}—, —C(O)—, or C₁₋₃ alkylene, wherein the alkylene is optionally substituted with one to two R^{Lb}. In some embodiments of a compound of Formula I, II, III, IV, V, VI, or VII, or a pharmaceutically acceptable salt thereof, L is a bond, —C(O)NR^{La}—, —C(O)—, or C₁₋₃ alkylene, wherein the alkylene is optionally substituted with two R^{Lb}. In some embodiments of a compound of Formula I, II, III, IV, V, VI, or VII, or a pharmaceutically acceptable salt thereof, L is a bond, —C(O)NR^{La}—, —C(O)—, or C₁₋₃ alkylene, wherein the alkylene is optionally substituted with one R^{Lb}.

In some embodiments of a compound of Formula I, II, III, IV, V, VI, or VII, or a pharmaceutically acceptable salt thereof, L is a bond, —C(O)NR^{La}—, —C(O)—, or C₁₋₃ alkylene, wherein the alkylene is substituted with one to three R^{Lb}. In some embodiments of a compound of Formula I, II, III, IV, V, VI, or VII, or a pharmaceutically acceptable salt thereof, L is a bond, —C(O)NR^{La}, —C(O)—, or C₁₋₃ alkylene, wherein the alkylene is substituted with one to two R^{Lb}. In some embodiments of a compound of Formula I, II, III, IV, V, VI, or VII, or a pharmaceutically acceptable salt thereof, L is a bond, —C(O)NR^{La}, —C(O)—, or C₁₋₃ alkylene, wherein the alkylene is substituted with two R^{Lb}. In some embodiments of a compound of Formula I, II, III, IV, V, VI, or VII, or a pharmaceutically acceptable salt thereof, L is a bond, —C(O)NR^{La}—, —C(O)—, or C₁₋₃ alkylene, wherein the alkylene is substituted with one R^{Lb}.

In some embodiments of a compound of Formula I, II, III, IV, V, VI, or VII, or a pharmaceutically acceptable salt thereof, L is a bond, —O—, —NR^{La}—, —C(O)NR^{La}—, —C(O), or C₁₋₃ alkylene.

In some embodiments of a compound of Formula I, II, III, IV, V, VI, or VII, or a pharmaceutically acceptable salt thereof, L is —C(O)NR^{La}— or —C(O)—. In some embodiments of a compound of Formula I, II, III, IV, V, VI, or VII, or a pharmaceutically acceptable salt thereof, L is a bond. In some embodiments of a compound of Formula I, II, III, IV, V, VI, or VII, or a pharmaceutically acceptable salt thereof, L is —C(O)NR^{La}—. In some embodiments of a compound of Formula I, II, III, IV, V, VI, or VII, or a pharmaceutically acceptable salt thereof, L is —C(O)—. In some embodiments of a compound of Formula I, II, III, IV, V, VI, or VII, or a pharmaceutically acceptable salt thereof, L is C₁₋₃ alkylene. In some embodiments of a compound of Formula I, II, III, IV, V, VI, or VII, or a pharmaceutically acceptable salt thereof, L is ethylene. In some embodiments of a compound of Formula I, II, III, IV, V, VI, or VII, or a pharmaceutically acceptable salt thereof, L is methylene.

In some embodiments of a compound of Formula I, II, III, IV, V, VI, or VII, or a pharmaceutically acceptable salt thereof, each R^{La} is independently H, C₁₋₆ alkyl, C₁₋₆ haloalkyl, or C₃₋₁₀ cycloalkyl.

In some embodiments of a compound of Formula I, II, III, IV, V, VI, or VII, or a pharmaceutically acceptable salt thereof, each R^{La} is independently H or C₁₋₆ alkyl. In some embodiments of a compound of Formula I, II, III, IV, V, VI, or VII, or a pharmaceutically acceptable salt thereof, each R^{La} is independently H or C₁₋₃ alkyl. In some embodiments of a compound of Formula I, II, III, IV, V, VI, or VII, or a pharmaceutically acceptable salt thereof, each R^{La} is independently H or methyl.

In some embodiments of a compound of Formula I, II, III, IV, V, VI, or VII, or a pharmaceutically acceptable salt thereof, each R^{La} is H. In some embodiments of a compound of Formula I, II, III, IV, V, VI, or VII, or a pharmaceutically acceptable salt thereof, at least one R^{La} is H.

In some embodiments of a compound of Formula I, II, III, IV, V, VI, or VII, or a pharmaceutically acceptable salt thereof, each R^{La} is independently C₁₋₆ alkyl. In some embodiments of a compound of Formula I, II, III, IV, V, VI, or VII, or a pharmaceutically acceptable salt thereof, each R^{La} is independently C₁₋₃ alkyl. In some embodiments of a compound of Formula I, II, III, IV, V, VI, or VII, or a pharmaceutically acceptable salt thereof, each R^{La} is methyl.

In some embodiments of a compound of Formula I, II, III, IV, V, VI, or VII, or a pharmaceutically acceptable salt thereof, at least one R^{La} is C₁₋₆ alkyl. In some embodiments of a compound of Formula I, II, III, IV, V, VI, or VII, or a pharmaceutically acceptable salt thereof, at least one R^{La} is C₁₋₃ alkyl. In some embodiments of a compound of Formula I, II, III, IV, V, VI, or VII, or a pharmaceutically acceptable salt thereof, at least one R^{La} is methyl.

In some embodiments of a compound of Formula I, II, III, IV, V, VI, or VII, or a pharmaceutically acceptable salt thereof, each R^{Lb} is independently halogen, —OH, —OCH₃, —NH₂, or —CN

In some embodiments of a compound of Formula I, II, III, IV, V, VI, or VII, or a pharmaceutically acceptable salt thereof, each R is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is optionally substituted with one to three halogen. In some embodiments of a compound of Formula I, II, III, IV, V, VI, or VII, or a pharmaceutically acceptable salt thereof, each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is optionally substituted with one to two halogen. In some embodiments of a compound of Formula I, II, III, IV, V, VI, or VII, or a pharmaceutically acceptable salt thereof, each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is optionally substituted with two halogen. In some embodiments of a compound of Formula I, II, III, IV, V, VI, or VII, or a pharmaceutically acceptable salt thereof, each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is optionally substituted with one halogen.

In some embodiments of a compound of Formula I, II, III, IV, V, VI, or VII, or a pharmaceutically acceptable salt thereof, each R is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is substituted with one to three halogen. In some embodiments of a compound of Formula I, II, III, IV, V, VI, or VII, or a pharmaceutically acceptable salt thereof, each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is substituted with one to two halogen. In some embodiments of a compound of Formula I, II, III, IV, V, VI, or VII, or a pharmaceutically acceptable salt thereof, each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is substituted with two halogen. In some embodiments of a compound of Formula I, II, III, IV, V, VI, or VII, or a pharmaceutically acceptable salt thereof, each R is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is substituted with one halogen.

In some embodiments of a compound of Formula I, II, III, IV, V, VI, or VII, or a pharmaceutically acceptable salt thereof, each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl. In some embodiments of a compound of Formula I, II, III, IV, V, VI, or VII, or a pharmaceutically acceptable salt thereof, each R is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl. In some embodiments of a compound of Formula I, II, III, IV, V, VI, or VII, or a pharmaceutically acceptable salt thereof, each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl. In some embodiments of a compound of Formula I, II, III, IV, V, VI, or VII, or a pharmaceutically acceptable salt thereof, each R is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl.

In some embodiments of a compound of Formula I, II, III, IV, V, VI, or VII, or a pharmaceutically acceptable salt thereof, each R is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy. In some embodiments of a compound of Formula I, II, III, IV, V, VI, or VII, or a pharmaceutically acceptable salt thereof, each R^{Y} is independently halogen or C₁₋₆ alkyl. In some embodiments of a compound of Formula I, II, III, IV, V, VI, or VII, or a pharmaceutically acceptable salt thereof, each R^{Y} is independently halogen or C₁₋₃ alkyl. In some embodiments of a compound of Formula I, II, III, IV, V, VI, or VII, or a pharmaceutically acceptable salt thereof, each R^{Y} is independently halogen or methyl.

In some embodiments of a compound of Formula I, II, III, IV, V, VI, or VII, or a pharmaceutically acceptable salt thereof, each R^{Y} is independently chloro or C₁₋₆ alkyl. In some embodiments of a compound of Formula I, II, III, IV, V, VI, or VII, or a pharmaceutically acceptable salt thereof, each R^{Y} is independently chloro or C₁₋₃ alkyl. In some embodiments of a compound of Formula I, II, III, IV, V, VI, or VII, or a pharmaceutically acceptable salt thereof, each R^{Y} is independently chloro or methyl.

In some embodiments of a compound of Formula I, II, III, IV, V, VI, or VII, or a pharmaceutically acceptable salt thereof, each R^{Y} is independently fluoro or C₁₋₆ alkyl. In some embodiments of a compound of Formula I, II, III, IV, V, VI, or VII, or a pharmaceutically acceptable salt thereof, each R^{Y} is independently fluoro or C₁₋₃ alkyl. In some embodiments of a compound of Formula I, II, III, IV, V, VI, or VII, or a pharmaceutically acceptable salt thereof, each R is independently fluoro or methyl.

In some embodiments of a compound of Formula I, II, III, IV, V, VI, or VII, or a pharmaceutically acceptable salt thereof, each R is independently C₁₋₆ alkyl. In some embodiments of a compound of Formula I, II, III, IV, V, VI, or VII, or a pharmaceutically acceptable salt thereof, each R^{Y} is independently C₁₋₃ alkyl. In some embodiments of a compound of Formula I, II, III, IV, V, VI, or VII, or a pharmaceutically acceptable salt thereof, each R^{Y} is methyl.

In some embodiments of a compound of Formula I, II, III, IV, V, VI, or VII, or a pharmaceutically acceptable salt thereof, each R is independently halogen. In some embodiments of a compound of Formula I, II, III, IV, V, VI, or VII, or a pharmaceutically acceptable salt thereof, each R^{Y} is independently fluoro or chloro. In some embodiments of a compound of Formula I, II, III, IV, V, VI, or VII, or a pharmaceutically acceptable salt thereof, each R^{Y} is fluoro. In some embodiments of a compound of Formula I, II, III, IV, V, VI, or VII, or a pharmaceutically acceptable salt thereof, each R^{Y} is chloro.

In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl, wherein each cycloalkyl and heterocyclyl is optionally substituted with one to three halogen. In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl, wherein each cycloalkyl and heterocyclyl is optionally substituted with one to two halogen. In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl, wherein each cycloalkyl and heterocyclyl is optionally substituted with two halogen. In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl, wherein each cycloalkyl and heterocyclyl is optionally substituted with one halogen.

In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl, wherein each cycloalkyl and heterocyclyl is substituted with one to three halogen. In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl, wherein each cycloalkyl and heterocyclyl is substituted with one to two halogen.

In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl, wherein each cycloalkyl and heterocyclyl is substituted with two halogen. In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl, wherein each cycloalkyl and heterocyclyl is substituted with one halogen.

In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl. In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl. In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl. In some embodiments of a compound of Formula I, II, III, IV, V, VI, or VII, or a pharmaceutically acceptable salt thereof, R^{Y} and R¹ join together to form a C₃₋₆ cycloalkyl or 4- to 6-membered heterocyclyl.

In some embodiments of a compound of Formula I, II, III, IV, V, VI, or VII, or a pharmaceutically acceptable salt thereof, each R is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is optionally substituted with one to two halogen. In some embodiments of a compound of Formula I, II, III, IV, V, VI, or VII, or a pharmaceutically acceptable salt thereof, each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is optionally substituted with two halogen. In some embodiments of a compound of Formula I, II, III, IV, V, VI, or VII, or a pharmaceutically acceptable salt thereof, each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy; or R^{Y} and R¹ join together to form a C₃₋₆ cycloalkyl or 4- to 6-membered heterocyclyl, wherein each cycloalkyl and heterocyclyl is optionally substituted with one halogen.

In some embodiments of a compound of Formula I, II, III, IV, V, VI, or VII, or a pharmaceutically acceptable salt thereof, q is 0, 1, 2 or 3. In some embodiments of a compound of Formula I, II, III, IV, V, VI, or VII, or a pharmaceutically acceptable salt thereof, q is 1. In some embodiments of a compound of Formula I, II, III, IV, V, VI, or VII, or a pharmaceutically acceptable salt thereof, q is 0.

In some embodiments of a compound of Formula I, II, III, IV, V, VI, or VII, or a pharmaceutically acceptable salt thereof, X is CR³ or N. In some embodiments of a compound of Formula I, II, III, IV, V, VI, or VII, or a pharmaceutically acceptable salt thereof, X is CR³. In some embodiments of a compound of Formula I, II, III, IV, V, VI, or VII, or a pharmaceutically acceptable salt thereof, X is N.

In some embodiments of a compound of Formula I, II, III, IV, V, VI, or VII, or a pharmaceutically acceptable salt thereof, R³ is H, halogen, —CN, C₁₋₆ alkyl, C₁₋₆ haloalkyl, C₁₋₆ hydroxyalkyl, C₁₋₆ alkoxy, C₁₋₆ haloalkoxy, or —O(C₃₋₆ cycloalkyl). In some embodiments of a compound of Formula I, II, III, IV, V, VI, or VII, or a pharmaceutically acceptable salt thereof, R³ is H or halogen.

In some embodiments of a compound of Formula I, II, III, IV, V, VI, or VII, or a pharmaceutically acceptable salt thereof, R³ is H.

In some embodiments of a compound of Formula I, II, III, IV, V, VI, or VII, or a pharmaceutically acceptable salt thereof, R³ is halogen. In some embodiments of a compound of Formula I, II, III, IV, V, VI, or VII, or a pharmaceutically acceptable salt thereof, R³ is fluoro. In some embodiments of a compound of Formula I, II, III, IV, V, VI, or VII, or a pharmaceutically acceptable salt thereof, R³ is chloro.

In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl, wherein each alkyl, alkynyl, and cycloalkyl is optionally substituted with one to three R^{4a1}. In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl, wherein each alkyl, alkynyl, and cycloalkyl is optionally substituted with one to two R^{4a1}. In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl, wherein each alkyl, alkynyl, and cycloalkyl is optionally substituted with two R^{4a1}. In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl, wherein each alkyl, alkynyl, and cycloalkyl is optionally substituted with one R^{4a1}.

In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl, wherein each alkyl, alkynyl, and cycloalkyl is substituted with one to three R^{4a1}. In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl, wherein each alkyl, alkynyl, and cycloalkyl is substituted with one to two R^{4a1}. In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl, wherein each alkyl, alkynyl, and cycloalkyl is substituted with two R^{4a1}. In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl, wherein each alkyl, alkynyl, and cycloalkyl is substituted with one R^{4a1}.

In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl. In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl. In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl. In some embodiments of a compound of Formula I, II, III, IV, V, VI, or VII, or a pharmaceutically acceptable salt thereof, R⁴ is —H, —CN, C₁₋₆ alkyl, C₂₋₆ alkynyl, or C₃₋₁₀ cycloalkyl.

In some embodiments of a compound of Formula I, II, III, IV, V, VI, or VII, or a pharmaceutically acceptable salt thereof, R⁴ is H, —CN, or C₁₋₆ alkyl wherein the alkyl is optionally substituted with one to three R^{4a1}. In some embodiments of a compound of Formula I, II, III, IV, V, VI, or VII, or a pharmaceutically acceptable salt thereof, R⁴ is H, —CN, or C₁₋₆ alkyl wherein the alkyl is optionally substituted with one to two R^{4a1}. In some embodiments of a compound of Formula I, II, III, IV, V, VI, or VII, or a pharmaceutically acceptable salt thereof, R⁴ is H, —CN, or C₁₋₆ alkyl wherein the alkyl is optionally substituted with two R^{4a1}. In some embodiments of a compound of Formula I, II, III, IV, V, VI, or VII, or a pharmaceutically acceptable salt thereof, R⁴ is H, —CN, or C₁₋₆ alkyl wherein the alkyl is optionally substituted with one R^{4a1}.

In some embodiments of a compound of Formula I, II, III, IV, V, VI, or VII, or a pharmaceutically acceptable salt thereof, R⁴ is H, —CN, or C₁₋₆ alkyl wherein the alkyl is substituted with one to three R^{4a1}. In some embodiments of a compound of Formula I, II, III, IV, V, VI, or VII, or a pharmaceutically acceptable salt thereof, R⁴ is H, —CN, or C₁₋₆ alkyl wherein the alkyl is substituted with one to two R^{4a1}. In some embodiments of a compound of Formula I, II, III, IV, V, VI, or VII, or a pharmaceutically acceptable salt thereof, R⁴ is H, —CN, or C₁₋₆ alkyl wherein the alkyl is substituted with two R^{4a1}. In some embodiments of a compound of Formula I, II, III, IV, V, VI, or VII, or a pharmaceutically acceptable salt thereof, R⁴ is H, —CN, or C₁₋₆ alkyl wherein the alkyl is substituted with one R^{4a1}.

In some embodiments of a compound of Formula I, II, III, IV, V, VI, or VII, or a pharmaceutically acceptable salt thereof, R⁴ is H, —CN, or C₁₋₆ alkyl. In some embodiments of a compound of Formula I, II, III, IV, V, VI, or VII, or a pharmaceutically acceptable salt thereof, R⁴ is H, —CN, or C₁₋₆ alkyl. In some embodiments of a compound of Formula I, II, III, IV, V, VI, or VII, or a pharmaceutically acceptable salt thereof, R⁴ is H, —CN, or C₁₋₆ alkyl. In some embodiments of a compound of Formula I, II, III, IV, V, VI, or VII, or a pharmaceutically acceptable salt thereof, R⁴ is H, —CN, or C₁₋₆ alkyl.

In some embodiments of a compound of Formula I, II, III, IV, V, VI, or VII, or a pharmaceutically acceptable salt thereof, R⁴ is —CN or C₁₋₆ alkyl.

In some embodiments of a compound of Formula I, II, III, IV, V, VI, or VII, or a pharmaceutically acceptable salt thereof, R⁴ is —CN.

In some embodiments of a compound of Formula I, II, III, IV, V, VI, or VII, or a pharmaceutically acceptable salt thereof, R⁴ is C₁₋₆ alkyl. In some embodiments of a compound of Formula I, II, III, IV, V, VI, or VII, or a pharmaceutically acceptable salt thereof, R⁴ is C₁₋₃ alkyl. In some embodiments of a compound of Formula I, II, III, IV, V, VI, or VII, or a pharmaceutically acceptable salt thereof, R⁴ is methyl.

In some embodiments of a compound of Formula I, II, III, IV, V, VI, or VII, or a pharmaceutically acceptable salt thereof each R^{4a1} is independently halogen, —CN, —OR^{4a2}, or —NR^{4a2}R^{4a2}.

In some embodiments of a compound of Formula I, II, III, IV, V, VI, or VII, or a pharmaceutically acceptable salt thereof each R^{4a2} is independently H, C₁₋₆ alkyl, C₁₋₆ haloalkyl, C₃₋₆ cycloalkyl, —C(O)C₁₋₆ alkyl, —C(O)O(C₁₋₆ alkyl), or —C(O)N(R^{4a3})₂.

In some embodiments of a compound of Formula I, II, III, IV, V, VI, or VII, or a pharmaceutically acceptable salt thereof each R^{4a3} is independently C₁₋₆ alkyl.

In some embodiments, provided herein is a compound of is a compound having Formula (III)

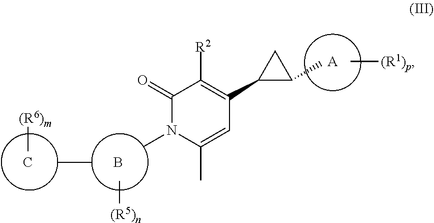

or a pharmaceutically acceptable salt thereof, wherein ring \textcircled{A}, R¹, R², ring \textcircled{B}, R⁵, ring \textcircled{C}, R⁶, m, n, and p are as defined for the compounds of Formula I described herein.

In some embodiments of a compound of Formula I, II, III, IV, V, VI, or VII, or a pharmaceutically acceptable salt thereof, R² is H, halogen, C₁₋₆ alkyl, or C₃₋₆ cycloalkyl, wherein each alkyl and cycloalkyl is optionally substituted with one to three halogen. In some embodiments of a compound of Formula I, II, III, IV, V, VI, or VII, or a pharmaceutically acceptable salt thereof, R² is H, halogen, or C₁₋₆ alkyl. In some embodiments of a compound of Formula I, II, III, IV, V, VI, or VII, or a pharmaceutically acceptable salt thereof, R² is H, halogen, or C₁₋₃ alkyl.

In some embodiments of a compound of Formula I, II, III, IV, V, VI, or VII, or a pharmaceutically acceptable salt thereof, R² is H or halogen. In some embodiments of a compound of Formula I, II, III, IV, V, VI, or VII, or a pharmaceutically acceptable salt thereof, R² is H, chloro, or bromo.

In some embodiments of a compound of Formula I, II, III, IV, V, VI, or VII, or a pharmaceutically acceptable salt thereof, R² is H.

In some embodiments of a compound of Formula I, II, III, IV, V, VI, or VII, or a pharmaceutically acceptable salt thereof, R² is halogen. In some embodiments of a compound of Formula I, II, III, IV, V, VI, or VII, or a pharmaceutically acceptable salt thereof, R² is chloro. In some embodiments of a compound of Formula I, II, III, IV, V, VI, or VII, or a pharmaceutically acceptable salt thereof, R² is bromo.

In some embodiments of a compound of Formula I, II, III, IV, V, VI, or VII, or a pharmaceutically acceptable salt thereof, ring \textcircled{B} is C₆₋₁₀ aryl or 5- to 10-membered heteroaryl.

In some embodiments of a compound of Formula I, II, III, IV, V, VI, or VII, or a pharmaceutically acceptable salt thereof, ring \textcircled{B} is C₆₋₁₀ aryl. In some embodiments of a compound of Formula I, II, III, IV, V, VI, or VII, or a pharmaceutically acceptable salt thereof, ring \textcircled{B} is phenyl.

In some embodiments of a compound of Formula I, II, III, IV, V, VI, or VII, or a pharmaceutically acceptable salt thereof, ring is 5- to 10-membered heteroaryl. In some embodiments of a compound of Formula I, II, III, IV, V, VI, or VII, or a pharmaceutically acceptable salt thereof, ring \textcircled{B} is 5- to 9-membered heteroaryl. In some embodiments of a compound of Formula I, II, III, IV, V, VI, or VII, or a pharmaceutically acceptable salt thereof, ring \textcircled{B} is 5- to 6-membered heteroaryl. In some embodiments of a compound of Formula I, II, III, IV, V, VI, or VII, or a pharmaceutically acceptable salt thereof, ring \textcircled{B} is 5-membered heteroaryl. In some embodiments of a compound of Formula I, II, III, IV, V, VI, or VII, or a pharmaceutically acceptable salt thereof, ring \textcircled{B} is 6-membered heteroaryl. In some embodiments of a compound of Formula I, II, III, IV, V, VI, or VII, or a pharmaceutically acceptable salt thereof, ring is 9-membered heteroaryl. In some embodiments of a compound of Formula I, II, III, IV, V, VI, or VII, or a pharmaceutically acceptable salt thereof, ring \textcircled{B} is 10-membered heteroaryl.

In some embodiments of a compound of Formula I, II, III, IV, V, VI, or VII, or a pharmaceutically acceptable salt thereof, ring \textcircled{B} is pyridinyl, pyrimidinyl, pyrazinyl, or pyridazinyl. In some embodiments of a compound of Formula I, II, III, IV, V, VI, or VII, or a pharmaceutically acceptable salt thereof, ring \textcircled{B} is pyridinyl.

In some embodiments, provided herein is a compound of is a compound having Formula (IV)

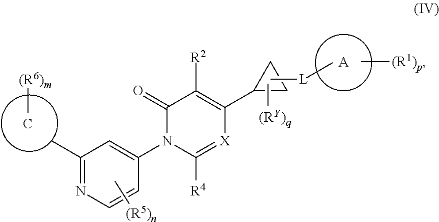

or a pharmaceutically acceptable salt thereof, wherein ring \textcircled{A}, R¹, L, R^{Y}, X, R², R³, R⁴, R⁵, ring \textcircled{C}, R⁶, m, n, p, and q are as defined for the compounds of Formula I described herein.

In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one to three R^{5b1}. In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one to two R^{5b1}. In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with two R^{5b1}. In some embodiments of a compound of Formula I, II, III, IV, V, VI, or VII, or a pharmaceutically acceptable salt thereof, each R is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one R^{5b1}.

In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one to three R^{5b1}. In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one to two R^{5b1}. In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with two R^{5b1} In some embodiments of a compound of Formula I, II, III, IV, V, VI, or VII, or a pharmaceutically acceptable salt thereof, each R is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one R^{5b1}.

In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is substituted with one to three R^{5b1}. In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is substituted with one to two R^{5b1}. In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is substituted with two R^{5b1}. In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is substituted with one R^{5b1}.

In some embodiments of a compound of Formula I, II, III, IV, V, VI, or VII, or a pharmaceutically acceptable salt thereof, each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}. In some embodiments of a compound of Formula I, II, III, IV, V, VI, or VII, or a pharmaceutically acceptable salt thereof, each R⁵ is independently halogen or C₁₋₆ alkyl. In some embodiments of a compound of Formula I, II, III, IV, V, VI, or VII, or a pharmaceutically acceptable salt thereof, each R⁵ is independently halogen or C₁₋₃ alkyl. In some embodiments of a compound of Formula I, II, III, IV, V, VI, or VII, or a pharmaceutically acceptable salt thereof, each R is independently chloro, fluoro, or methyl.

In some embodiments of a compound of Formula I, II, III, IV, V, VI, or VII, or a pharmaceutically acceptable salt thereof, each R⁵ is independently C₁₋₆ alkyl, wherein the alkyl is optionally substituted with one to three R^{5b1}. In some embodiments of a compound of Formula I, II, III, IV, V, VI, or VII, or a pharmaceutically acceptable salt thereof, each R⁵ is independently C₁₋₆ alkyl, wherein the alkyl is optionally substituted with one to two R^{5b1}. In some embodiments of a compound of Formula I, II, III, IV, V, VI, or VII, or a pharmaceutically acceptable salt thereof, each R⁵ is independently C₁₋₆ alkyl, wherein the alkyl is optionally substituted with two R^{5b1}. In some embodiments of a compound of Formula I, II, III, IV, V, VI, or VII, or a pharmaceutically acceptable salt thereof, each R⁵ is independently C₁₋₆ alkyl, wherein the alkyl is optionally substituted with one R^{5b1}.

In some embodiments of a compound of Formula I, II, III, IV, V, VI, or VII, or a pharmaceutically acceptable salt thereof, each R⁵ is independently C₁₋₆ alkyl, wherein the alkyl is substituted with one to three R^{5b1}. In some embodiments of a compound of Formula I, II, III, IV, V, VI, or VII, or a pharmaceutically acceptable salt thereof, each R⁵ is independently C₁₋₆ alkyl, wherein the alkyl is substituted with one to two R^{5b1}. In some embodiments of a compound of Formula I, II, III, IV, V, VI, or VII, or a pharmaceutically acceptable salt thereof, each R⁵ is independently C₁₋₆ alkyl, wherein the alkyl is substituted with two R^{5b1}. In some embodiments of a compound of Formula I, II, III, IV, V, VI, or VII, or a pharmaceutically acceptable salt thereof, each R⁵ is independently C₁₋₆ alkyl, wherein the alkyl is substituted with one R^{5b1}.

In some embodiments of a compound of Formula I, II, III, IV, V, VI, or VII, or a pharmaceutically acceptable salt thereof, each R⁵ is independently C₁₋₆ alkyl. In some embodiments of a compound of Formula I, II, III, IV, V, VI, or VII, or a pharmaceutically acceptable salt thereof, each R is independently C₁₋₃ alkyl. In some embodiments of a compound of Formula I, II, III, IV, V, VI, or VII, or a pharmaceutically acceptable salt thereof, R⁵ is methyl.

In some embodiments of a compound of Formula I, II, III, IV, V, VI, or VII, or a pharmaceutically acceptable salt thereof, each R⁵ is independently halogen. In some embodiments of a compound of Formula I, II, III, IV, V, VI, or VII, or a pharmaceutically acceptable salt thereof, each R⁵ is independently fluoro or chloro. In some embodiments of a compound of Formula I, II, III, IV, V, VI, or VII, or a pharmaceutically acceptable salt thereof, each R⁵ is fluoro. In some embodiments of a compound of Formula I, II, III, IV, V, VI, or VII, or a pharmaceutically acceptable salt thereof, each R is chloro.

In some embodiments of a compound of Formula I, II, III, IV, V, VI, or VII, or a pharmaceutically acceptable salt thereof each R^{5a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with one to three R^{5b3}In some embodiments of a compound of Formula I, II, III, IV, V, VI, or VII, or a pharmaceutically acceptable salt thereof each R^{5b1} is independently halogen, —CN, C₁₋₆ alkyl, C₂₋₆ alkynyl, C₁₋₆ haloalkyl, C₃₋₁₀ cycloalkyl, 4- to 10-membered heterocyclyl, —OR^{5b2}, or —N(R^{5b2})₂, wherein each alkyl, alkynyl, haloalkyl, cycloalkyl, and heterocycle is optionally substituted with one to three R^{5b3}In some embodiments of a compound of Formula I, II, III, IV, V, VI, or VII, or a pharmaceutically acceptable salt thereof each R^{5b2} is independently H, C₁₋₆ alkyl, or C₃₋₁₀ cycloalkyl, wherein each alkyl and cycloalkyl is optionally substituted with one or two oxo and is further optionally substituted with one to three R^{5b3}In some embodiments of a compound of Formula I, II, III, IV, V, VI, or VII, or a pharmaceutically acceptable salt thereof each R^{5b3} is independently halogen, —CN, —OH, —NH₂, C₁₋₆ alkyl, C₁₋₆ haloalkyl, C₁₋₆ hydroxyalkyl, C₁₋₆ alkoxy, C₁₋₆haloalkoxy, C₃₋₆ cycloalkyl, C₃₋₆ halocycloalkyl, or —O(C₃₋₆ cycloalkyl). In some embodiments of a compound of Formula I, II, III, IV, V, VI, or VII, or a pharmaceutically acceptable salt thereof each R^{5b3} is independently halogen. In some embodiments of a compound of Formula I, II, III, IV, V, VI, or VII, or a pharmaceutically acceptable salt thereof each R^{5b3} is independently chloro. In some embodiments of a compound of Formula I, II, III, IV, V, VI, or VII, or a pharmaceutically acceptable salt thereof each R^{5b3} is independently fluoro.

In some embodiments of a compound of Formula I, II, III, IV, V, VI, or VII, or a pharmaceutically acceptable salt thereof, n is 0, 1, 2, or 3. In some embodiments of a compound of Formula I, II, III, IV, V, VI, or VII, or a pharmaceutically acceptable salt thereof, n is 0, 1, or 2. In some embodiments of a compound of Formula I, II, III, IV, V, VI, or VII, or a pharmaceutically acceptable salt thereof, n is 1 or 2. In some embodiments of a compound of Formula I, II, III, IV, V, VI, or VII, or a pharmaceutically acceptable salt thereof, n is 0. In some embodiments of a compound of Formula I, II, III, IV, V, VI, or VII, or a pharmaceutically acceptable salt thereof, n is 1. In some embodiments of a compound of Formula I, II, III, IV, V, VI, or VII, or a pharmaceutically acceptable salt thereof, n is 2.

In some embodiments of a compound of Formula I, II, III, IV, V, VI, or VII, or a pharmaceutically acceptable salt thereof, ring \textcircled{B} is

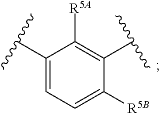

and each R^{5A} and R^{5B} is independently H or R⁵. In some embodiments of a compound of Formula I, II, III, IV, V, VI, or VII, or a pharmaceutically acceptable salt thereof, each R^{5A} and R^{5B} is independently H or R⁵. In some embodiments of a compound of Formula I, II, III, IV, V, VI, or VII, or a pharmaceutically acceptable salt thereof, each R^{5A} and R^{5B} is H. In some embodiments of a compound of Formula I, II, III, IV, V, VI, or VII, or a pharmaceutically acceptable salt thereof, each R^{5A} and R^{5B} is R. In some embodiments of a compound of Formula I, II, III, IV, V, VI, or VII, or a pharmaceutically acceptable salt thereof, each R^{5A} is H. In some embodiments of a compound of Formula I, II, III, IV, V, VI, or VII, or a pharmaceutically acceptable salt thereof, each R^{5B} is R⁵.

In some embodiments of a compound of Formula I, II, III, IV, V, VI, or VII, or a pharmaceutically acceptable salt thereof, ring \textcircled{B} is

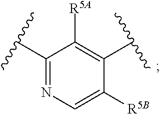

and each R^{5A} and R^{5B} is independently H or R⁵. In some embodiments of a compound of Formula I, II, III, IV, V, VI, or VII, or a pharmaceutically acceptable salt thereof, each R^{5A} and R^{5B} is independently H or R⁵. In some embodiments of a compound of Formula I, II, III, IV, V, VI, or VII, or a pharmaceutically acceptable salt thereof, each R^{5A} and R^{5B} is H. In some embodiments of a compound of Formula I, II, III, IV, V, VI, or VII, or a pharmaceutically acceptable salt thereof, each R^{5A} and R^{5B} is R⁵. In some embodiments of a compound of Formula I, II, III, IV, V, VI, or VII, or a pharmaceutically acceptable salt thereof, each R^{5A} is H. In some embodiments of a compound of Formula I, II, III, IV, V, VI, or VII, or a pharmaceutically acceptable salt thereof, each R^{5B} is R⁵.

In some embodiments, provided herein is a compound of is a compound having Formula (V)

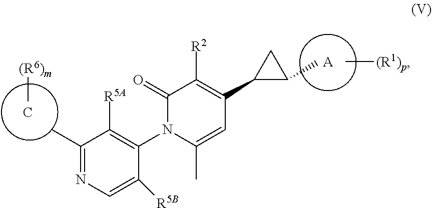

or a pharmaceutically acceptable salt thereof, wherein ring, R¹, R², R^{5A}, R^{5B} ring \textcircled{C}, R⁶, m, and p are as defined for the compounds of Formula I described herein.

In some embodiments of a compound of Formula I, II, III, IV, V, VI, or VII, or a pharmaceutically acceptable salt thereof R^{5B} is halogen, C₁₋₆ alkyl, or C₂₋₆ alkynyl, wherein each alkyl and alkynyl is optionally substituted with one to three halogen. In some embodiments of a compound of Formula I, II, III, IV, V, VI, or VII, or a pharmaceutically acceptable salt thereof R^{5B} is halogen, C₁₋₆ alkyl, or C₂₋₆ alkynyl. In some embodiments of a compound of Formula I, II, III, IV, V, VI, or VII, or a pharmaceutically acceptable salt thereof R^{5B} is methyl, —CF₃, CHF₂, or —C≡CCH₃.

In some embodiments of a compound of Formula I, II, III, IV, V, VI, or VII, or a pharmaceutically acceptable salt thereof R^{5B} is halogen. In some embodiments of a compound of Formula I, II, III, IV, V, VI, or VII, or a pharmaceutically acceptable salt thereof R^{5B} is chloro.

In some embodiments of a compound of Formula I, II, III, IV, V, VI, or VII, or a pharmaceutically acceptable salt thereof R^{5B} is C₁₋₆ alkyl. In some embodiments of a compound of Formula I, II, III, IV, V, VI, or VII, or a pharmaceutically acceptable salt thereof R^{5B} is C₁₋₃ alkyl. In some embodiments of a compound of Formula I, II, III, IV, V, VI, or VII, or a pharmaceutically acceptable salt thereof R^{5B} is methyl.

In some embodiments of a compound of Formula I, II, III, IV, V, VI, or VII, or a pharmaceutically acceptable salt thereof R^{5B} is H.

In some embodiments of a compound of Formula I, II, III, IV, V, VI, or VII, or a pharmaceutically acceptable salt thereof ring \textcircled{B} is

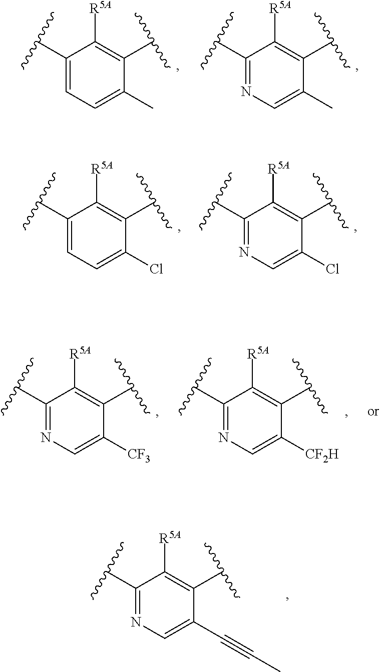

wherein R^{5A} is as defined for the compounds of Formula I, II, III, IV, V, VI, or VII escribe herein. In some embodiments of a compound of Formula I, II, III, IV, V, VI, or VII, or a pharmaceutically acceptable salt thereof R^{5A} is halogen. In some embodiments of a compound of Formula I, II, III, IV, V, VI, or VII, or a pharmaceutically acceptable salt thereof R^{5A} is fluoro. In some embodiments of a compound of Formula I, II, III, IV, V, VI, or VII, or a pharmaceutically acceptable salt thereof R^{5A} is H.

In some embodiments of a compound of Formula I, II, III, IV, V, VI, or VII, or a pharmaceutically acceptable salt thereof ring \textcircled{B} is

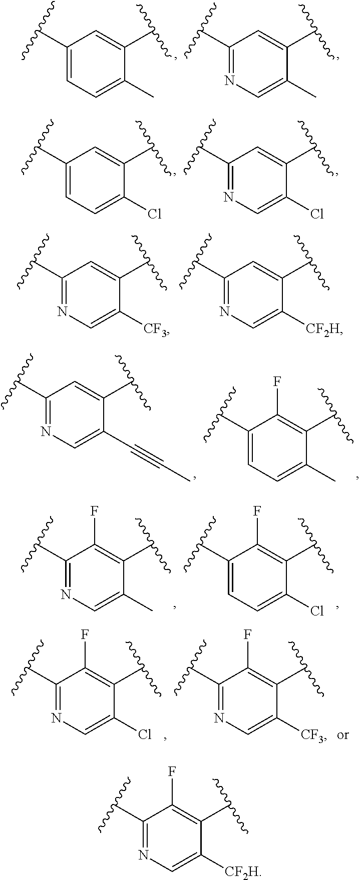

In some embodiments of a compound of Formula I, II, III, IV, V, VI, or VII, or a pharmaceutically acceptable salt thereof ring \textcircled{B} is

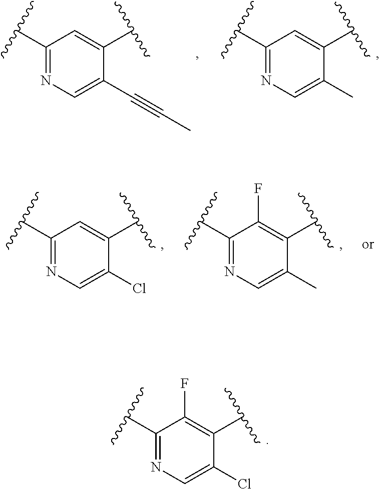

In some embodiments of a compound of Formula I, II, III, IV, V, VI, or VII, or a pharmaceutically acceptable salt thereof ring \textcircled{B} is

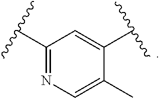 [SMILES: *c1cc(*)c(C)cn1]

In some embodiments of a compound of Formula I, II, III, IV, V, VI, or VII, or a pharmaceutically acceptable salt thereof ring \textcircled{B} is

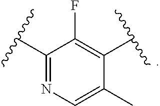 [SMILES: *c1ncc(C)c(*)c1F]

In some embodiments of a compound of Formula I, II, III, IV, V, VI, or VII, or a pharmaceutically acceptable salt thereof ring \textcircled{C} is C₆₋₁₀ aryl, 5- to 10-membered heteroaryl, C₃₋₈ cycloalkyl, or 4- to 10-membered heterocyclyl.

In some embodiments of a compound of Formula I, II, III, IV, V, VI, or VII, or a pharmaceutically acceptable salt thereof ring \textcircled{C} is C₆₋₁₀ aryl. In some embodiments of a compound of Formula I, II, III, IV, V, VI, or VII, or a pharmaceutically acceptable salt thereof ring \textcircled{C} is phenyl.

In some embodiments of a compound of Formula I, II, III, IV, V, VI, or VII, or a pharmaceutically acceptable salt thereof ring \textcircled{C} is 5- to 10-membered heteroaryl. In some embodiments of a compound of Formula I, II, III, IV, V, VI, or VII, or a pharmaceutically acceptable salt thereof ring \textcircled{C} is 5- to 6-membered heteroaryl. In some embodiments of a compound of Formula I, II, III, IV, V, VI, or VII, or a pharmaceutically acceptable salt thereof ring is 5-membered heteroaryl. In some embodiments of a compound of Formula I, II, III, IV, V, VI, or VII, or a pharmaceutically acceptable salt thereof ring \textcircled{C} is 6-membered heteroaryl.

In some embodiments of a compound of Formula I, II, III, IV, V, VI, or VII, or a pharmaceutically acceptable salt thereof ring \textcircled{C} is

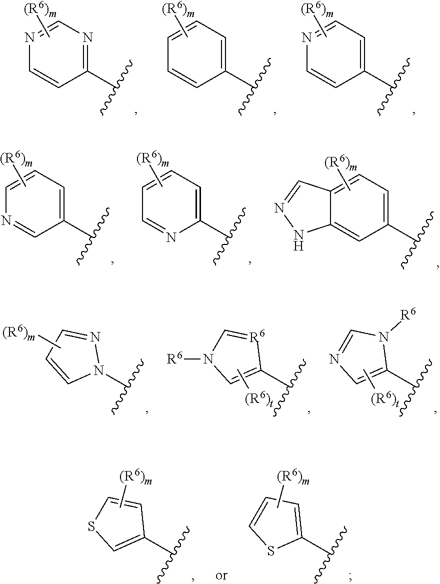

wherein R⁶ and m are as defined for the compounds of Formula I, II, III, IV, V, VI, or VII described herein, and t is 0, 1, or 2. In some embodiments of a compound of Formula I, II, III, IV, V, VI, or VII, or a pharmaceutically acceptable salt thereof, t is 0 or 1. In some embodiments of a compound of Formula I, II, III, IV, V, VI, or VII, or a pharmaceutically acceptable salt thereof, t is 0. In some embodiments of a compound of Formula I, II, III, IV, V, VI, or VII, or a pharmaceutically acceptable salt thereof, to is 1. In some embodiments of a compound of Formula I, II, III, IV, V, VI, or VII, or a pharmaceutically acceptable salt thereof, m is 0, 1, 2, or 3. In some embodiments of a compound of Formula I, II, III, IV, V, VI, or VII, or a pharmaceutically acceptable salt thereof m is 0, 1, or 2. In some embodiments of a compound of Formula I, II, III, IV, V, VI, or VII, or a pharmaceutically acceptable salt thereof m is 0. In some embodiments of a compound of Formula I, II, III, IV, V, VI, or VII, or a pharmaceutically acceptable salt thereof m is 1. In some embodiments of a compound of Formula I, II, III, IV, V, VI, or VII, or a pharmaceutically acceptable salt thereof m is 2.

In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further optionally substituted with one to three R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further optionally substituted with one to two R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further optionally substituted with two R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further optionally substituted with one R^{6b1}.

In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further substituted with one to three R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further substituted with one to two R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further substituted with two R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is substituted with one to three halogen and is further optionally substituted with one R^{6b1}.

In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl.

In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further optionally substituted with one to three R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further optionally substituted with one to two R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further optionally substituted with two R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further optionally substituted with one R^{6b1}.

In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further substituted with one to three R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further substituted with one to two R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further substituted with two R^{6b1}. In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further substituted with one R^{6b1}.

In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen.

In some embodiments of a compound of Formula I, II, III, IV, V, VI, or VII, or a pharmaceutically acceptable salt thereof each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1}12, —C(O)N(R^{6a1})S(O)₂R^{6a1}, —S(O)₂N(R^{6a1})C(O)R^{6a1}, or —P(O)(R^{6a1})₂.

In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to three R^{6b1}. In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to two R^{6b1}. In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with two R^{6b1}. In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one R^{6b1}.

In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one to three R^{6b1}. In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one to two R^{6b1}. In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with two R^{6b1}. In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —OR^{6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one R^{6b1}.

In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen.

In some embodiments of a compound of Formula I, II, III, IV, V, VI, or VII, or a pharmaceutically acceptable salt thereof each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂.

In some embodiments of a compound of Formula I, II, III, IV, V, VI, or VII, or a pharmaceutically acceptable salt thereof each R⁶ is independently halogen, —CN, —C₁₋₆ alkyl, 4- to 10-membered heterocyclyl, 5- to 10-membered heteroaryl, —C(O)N(R^{6a1})(R^{6a1}), —N(R^{6a1})C(O)—R^{6a1}, —N(R^{6a1})C(O)O—R^{6a1}, N═S(O)(R^{6a1})₂, —S(NR^{6a1})(O)(R^{6a1}), or —S(O)₂R^{6a1}.

In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with on e to three R^{6b1}. In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to two R^{6b1}. In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with two R^{6b1}. In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one R^{6b1}.

In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one to three R^{6b1}. In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one to two R^{6b1}. In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with two R^{6b1}. In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one R^{6b1}.

In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen.

In some embodiments of a compound of Formula I, II, III, IV, V, VI, or VII, or a pharmaceutically acceptable salt thereof each R⁶ is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂.

In some embodiments of a compound of Formula I, II, III, IV, V, VI, or VII, or a pharmaceutically acceptable salt thereof each R⁶ is independently halogen. In some embodiments of a compound of Formula I, II, III, IV, V, VI, or VII, or a pharmaceutically acceptable salt thereof each R⁶ is independently chloro or fluoro. In some embodiments of a compound of Formula I, II, III, IV, V, VI, or VII, or a pharmaceutically acceptable salt thereof each R⁶ is chloro. In some embodiments of a compound of Formula I, II, III, IV, V, VI, or VII, or a pharmaceutically acceptable salt thereof each R⁶ is fluoro.

In some embodiments of a compound of Formula I, II, III, IV, V, VI, or VII, or a pharmaceutically acceptable salt thereof each R⁶ is independently 5- to 6-membered heterocyclyl or 5- to 6-membered heteroaryl wherein each heterocyclyl and heteroaryl is optionally substituted with one to three halogen and is further substituted optionally with one to three R^{6b1}.

In some embodiments of a compound of Formula I, II, III, IV, V, VI, or VII, or a pharmaceutically acceptable salt thereof each R⁶ is independently 5- to 6-membered heterocyclyl, wherein the heterocyclyl is optionally substituted with one to three halogen and is further optionally substituted with one to three R^{6b1}. In some embodiments of a compound of Formula I, II, III, IV, V, VI, or VII, or a pharmaceutically acceptable salt thereof each R⁶ is independently 5- to 6-membered heterocyclyl, wherein the heterocyclyl is optionally substituted with one to three halogen and is further optionally substituted with one to two R^{6b1}. In some embodiments of a compound of Formula I, II, III, IV, V, VI, or VII, or a pharmaceutically acceptable salt thereof each R⁶ is independently 5- to 6-membered heterocyclyl, wherein the heterocyclyl is optionally substituted with one to three halogen and is further optionally substituted with two R^{6b1}. In some embodiments of a compound of Formula I, II, III, IV, V, VI, or VII, or a pharmaceutically acceptable salt thereof each R⁶ is independently 5- to 6-membered heterocyclyl, wherein the heterocyclyl is optionally substituted with one to three halogen and is further optionally substituted with one R^{6b1}.

In some embodiments of a compound of Formula I, II, III, IV, V, VI, or VII, or a pharmaceutically acceptable salt thereof each R⁶ is independently 5- to 6-membered heterocyclyl, wherein the heterocyclyl is optionally substituted with one to three halogen and is further substituted with one to three R^{6b1}. In some embodiments of a compound of Formula I, II, III, IV, V, VI, or VII, or a pharmaceutically acceptable salt thereof each R⁶ is independently 5- to 6-membered heterocyclyl, wherein the heterocyclyl is optionally substituted with one to three halogen and is further substituted with one to two R^{6b1}. In some embodiments of a compound of Formula I, II, III, IV, V, VI, or VII, or a pharmaceutically acceptable salt thereof each R⁶ is independently 5- to 6-membered heterocyclyl, wherein the heterocyclyl is optionally substituted with one to three halogen and is further substituted with two R^{6b1}. In some embodiments of a compound of Formula I, II, III, IV, V, VI, or VII, or a pharmaceutically acceptable salt thereof each R⁶ is independently 5- to 6-membered heterocyclyl, wherein the heterocyclyl is optionally substituted with one to three halogen and is further substituted with one R^{6b1}.

In some embodiments of a compound of Formula I, II, III, IV, V, VI, or VII, or a pharmaceutically acceptable salt thereof each R⁶ is independently 5- to 6-membered heteroaryl, wherein the heteroaryl is optionally substituted with one to three halogen and is further optionally substituted with one to three R^{6b1}. In some embodiments of a compound of Formula I, II, III, IV, V, VI, or VII, or a pharmaceutically acceptable salt thereof each R⁶ is independently 5- to 6-membered heteroaryl, wherein the heteroaryl is optionally substituted with one to three halogen and is further optionally substituted with one to two R^{6b1}. In some embodiments of a compound of Formula I, II, III, IV, V, VI, or VII, or a pharmaceutically acceptable salt thereof each R⁶ is independently 5- to 6-membered heteroaryl, wherein the heteroaryl is optionally substituted with one to three halogen and is further optionally substituted with two R^{6b1}. In some embodiments of a compound of Formula I, II, III, IV, V, VI, or VII, or a pharmaceutically acceptable salt thereof each R⁶ is independently 5- to 6-membered heteroaryl, wherein the heteroaryl is optionally substituted with one to three halogen and is further optionally substituted with one R^{6b1}.

In some embodiments of a compound of Formula I, II, III, IV, V, VI, or VII, or a pharmaceutically acceptable salt thereof each R⁶ is independently 5- to 6-membered heteroaryl, wherein the heteroaryl is optionally substituted with one to three halogen and is further substituted with one to three R^{6b1}. In some embodiments of a compound of Formula I, II, III, IV, V, VI, or VII, or a pharmaceutically acceptable salt thereof each R^{1A} is independently 5- to 6-membered heteroaryl, wherein the heteroaryl is optionally substituted with one to three halogen and is further substituted with one to two R^{6b1}. In some embodiments of a compound of Formula I, II, III, IV, V, VI, or VII, or a pharmaceutically acceptable salt thereof each R⁶ is independently 5- to 6-membered heteroaryl, wherein the heteroaryl is optionally substituted with one to three halogen and is further substituted with two R^{6b1}.

In some embodiments of a compound of Formula I, II, III, IV, V, VI, or VII, or a pharmaceutically acceptable salt thereof each R⁶ is independently 5- to 6-membered heteroaryl, wherein the heteroaryl is optionally substituted with one to three halogen and is further substituted with one R^{6b1}.

In some embodiments of a compound of Formula I, II, III, IV, V, VI, or VII, or a pharmaceutically acceptable salt thereof m is 0, 1, 2 or 3. In some embodiments of a compound of Formula I, II, III, IV, V, VI, or VII, or a pharmaceutically acceptable salt thereof m is 0, 1, or 2. In some embodiments of a compound of Formula I, II, III, IV, V, VI, or VII, or a pharmaceutically acceptable salt thereof m is 0. In some embodiments of a compound of Formula I, II, III, IV, V, VI, or VII, or a pharmaceutically acceptable salt thereof m is 1. In some embodiments of a compound of Formula I, II, III, IV, V, VI, or VII, or a pharmaceutically acceptable salt thereof m is 2. In some embodiments of a compound of Formula I, II, III, IV, V, VI, or VII, or a pharmaceutically acceptable salt thereof m is 3.

In some embodiments of a compound of Formula I, II, III, IV, V, VI, or VII, or a pharmaceutically acceptable salt thereof ring \textcircled{C} is

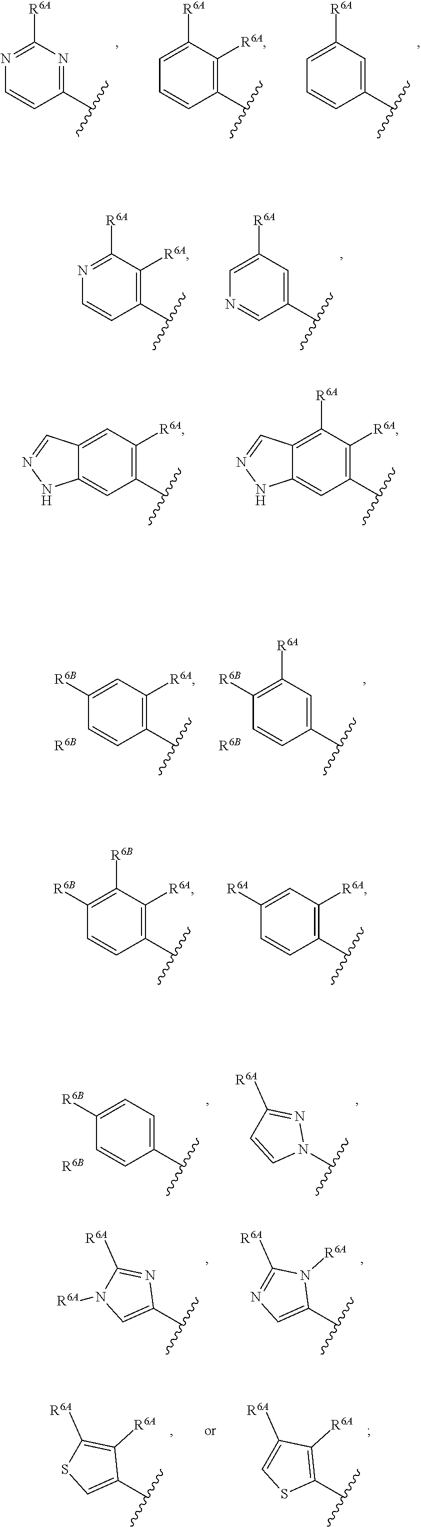

wherein
each R^{6A} is independently oxo, halogen, —NO₂, —N₃, —CN, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three R^{6b1}; and two R^{6B} groups taken together with two adjacent atoms of ring \textcircled{C} to which they are attached forma 4- to 10-membered heterocyclyl, wherein the heterocyclyl is optionally substituted with one to three R^{6b1}.

In some embodiments of a compound of Formula I, II, III, IV, V, VI, or VII, or a pharmaceutically acceptable salt thereof two R^{6B} groups taken together with two adjacent atoms of ring \textcircled{C} to which they are attached form a 4- to 10-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6B} groups taken together with two adjacent atoms of ring \textcircled{C} to which they are attached form a 4- to 6-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6B} groups taken together with two adjacent atoms of ring \textcircled{C} to which they are attached form a 5- to 6-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6B} groups taken together with two adjacent atoms of ring \textcircled{C} to which they are attached form a 5- to 6-membered heterocyclyl, wherein the heterocyclyl is optionally substituted with one to three R^{6b1}.

In some embodiments of a compound of Formula I, II, III, IV, V, VI, or VII, or a pharmaceutically acceptable salt thereof two R^{6B} groups taken together with two adjacent atoms of ring \textcircled{C} to which they are attached form a 4- to 10-membered heterocyclyl. In some embodiments of a compound of Formula I, II, III, IV, V, VI, or VII, or a pharmaceutically acceptable salt thereof two R^{6B} groups taken together with two adjacent atoms of ring \textcircled{C} to which they are attached form a 4- to 6-membered heterocyclyl. In some embodiments of a compound of Formula I, II, III, IV, V, VI, or VII, or a pharmaceutically acceptable salt thereof two R^{6B} groups taken together with two adjacent atoms of ring \textcircled{C} to which they are attached form a 5- to 6-membered heterocyclyl. In some embodiments of a compound of Formula I, II, III, IV, V, VI, or VII, or a pharmaceutically acceptable salt thereof two R^{6B} groups taken together with two adjacent atoms of ring \textcircled{C} to which they are attached form a 5- to 6-membered heterocyclyl.

In some embodiments of a compound of Formula I, II, III, IV, V, VI, or VII, or a pharmaceutically acceptable salt thereof ring \textcircled{C} is

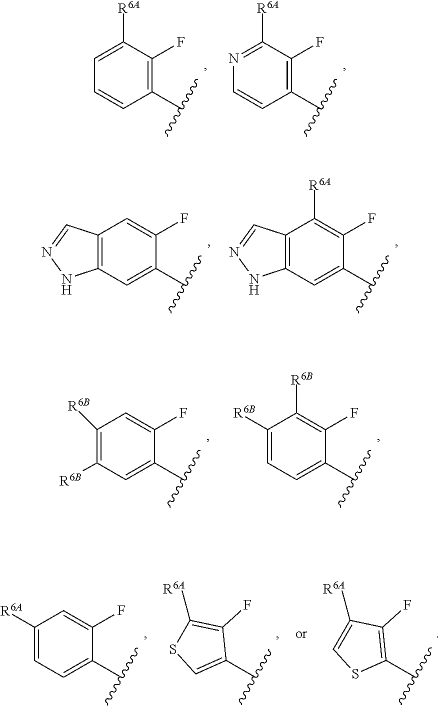

In some embodiments of a compound of Formula I, II, III, IV, V, VI, or VII, or a pharmaceutically acceptable salt thereof ring \textcircled{C} is

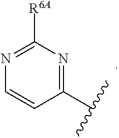

In some embodiments of a compound of Formula I, II, III, IV, V, VI, or VII, or a pharmaceutically acceptable salt thereof ring \textcircled{C} is.

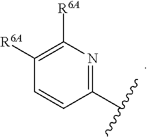

In some embodiments of a compound of Formula I, II, III, IV, V, VI, or VII, or a pharmaceutically acceptable salt thereof ring \textcircled{C} is,

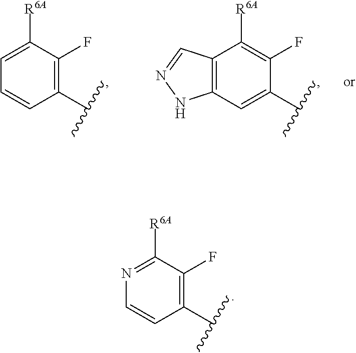

In some embodiments of a compound of Formula I, II, III, IV, V, VI, or VII, or a pharmaceutically acceptable salt thereof ring \textcircled{C} is

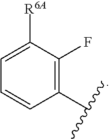

In some embodiments, provided herein is a compound of is a compound having Formula (VI)

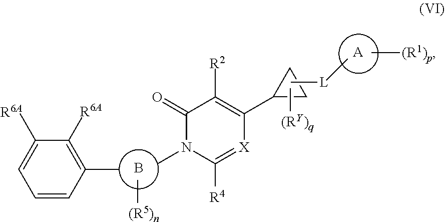

or a pharmaceutically acceptable salt thereof, wherein ring \textcircled{A}, R¹, L, R^{Y}, X, R², R³, R⁴ ring \textcircled{B}, R⁵, n, p, and q are as defined for the compounds of Formula I described herein; and each R^{6A} is independently H or R⁶.

In some embodiments, provided herein is a compound of is a compound having Formula (VII)

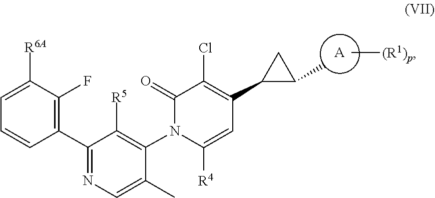

or a pharmaceutically acceptable salt thereof, wherein ring \textcircled{A}, R¹, R², X, R², R³, R⁴, R⁵, and p are as defined for the compounds of Formula I described herein; and each R^{6A} is independently H or R⁶.

In some embodiments of a compound of Formula I, or a pharmaceutically acceptable salt thereof each R^{6A} is independently H or R⁶. In some embodiments of a compound of Formula I, or a pharmaceutically acceptable salt thereof at least one R^{6A} is H. In some embodiments of a compound of Formula I, or a pharmaceutically acceptable salt thereof at least one R^{6A} is R⁶.

In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further optionally substituted with one to three R^{6b1}. In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further optionally substituted with one to two R^{6b1}. In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further optionally substituted with two R^{6b1}. In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further optionally substituted with one R^{6b1}.

In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further substituted with one to three R^{6b1}. In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further substituted with one to two R^{6b1}. In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further substituted with two R^{6b1}. In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen and is further substituted with one R^{6b1}.

In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three halogen.

In some embodiments of a compound of Formula I, or a pharmaceutically acceptable salt thereof each R^{6A} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂.

In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to three R^{6b1}. In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to two R^{6b1}. In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with two R^{6b1}. In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one R^{6b1}.

In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one to three R^{6b1}. In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —OR^{6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one to two R^{6b1}. In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with two R^{6b1}. In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —OR^{6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one R^{6b1}.

In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen.

In some embodiments of a compound of Formula I, or a pharmaceutically acceptable salt thereof each R^{6A} is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂.

In some embodiments of a compound of Formula I, or a pharmaceutically acceptable salt thereof each R^{6A} is independently halogen, —CN, —C₁₋₆ alkyl, 4- to 10-membered heterocyclyl, 5- to 10-membered heteroaryl, —C(O)N(R^{6a1})(R^{6a1}), —N(R^{6a1})C(O)—R^{6a1}, —N(R^{6a1})C(O)O—R^{6a1}, N═S(O)(R^{6a1})₂, —S(NR^{6a1})(O)(R^{6a1}), or —S(O)₂R^{6a1}.

In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to three R^{6b1}. In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to two R^{6b1}. In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with two R^{6b1}. In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one R^{6b1}.

In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one to three R^{6b1}. In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one to two R^{6b1}. In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with two R^{6b1}. In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further substituted with one R^{6b1}.

In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen.

In some embodiments of a compound of Formula I, or a pharmaceutically acceptable salt thereof each R^{6A} is independently C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂.

In some embodiments of a compound of Formula I, or a pharmaceutically acceptable salt thereof each R^{6A} is independently halogen. In some embodiments of a compound of Formula I, or a pharmaceutically acceptable salt thereof each R^{6A} is independently chloro or fluoro. In some embodiments of a compound of Formula I, or a pharmaceutically acceptable salt thereof each R^{6A} is chloro. In some embodiments of a compound of Formula I, or a pharmaceutically acceptable salt thereof each R^{6A} is fluoro.

In some embodiments of a compound of Formula I, or a pharmaceutically acceptable salt thereof each R^{6A} is independently 5- to 6-membered heterocyclyl or 5- to 6-membered heteroaryl wherein each heterocyclyl and heteroaryl is optionally substituted with one to three halogen and is further substituted optionally with one to three R^{6b1}.

In some embodiments of a compound of Formula I, or a pharmaceutically acceptable salt thereof each R^{6A} is independently 5- to 6-membered heterocyclyl, wherein the heterocyclyl is optionally substituted with one to three halogen and is further optionally substituted with one to three R^{6b1}. In some embodiments of a compound of Formula I, or a pharmaceutically acceptable salt thereof each R^{6A} is independently 5- to 6-membered heterocyclyl, wherein the heterocyclyl is optionally substituted with one to three halogen and is further optionally substituted with one to two R^{6b1}. In some embodiments of a compound of Formula I, or a pharmaceutically acceptable salt thereof each R^{6A} is independently 5- to 6-membered heterocyclyl, wherein the heterocyclyl is optionally substituted with one to three halogen and is further optionally substituted with two R^{6b1}. In some embodiments of a compound of Formula I, or a pharmaceutically acceptable salt thereof each R^{6A} is independently 5- to 6-membered heterocyclyl, wherein the heterocyclyl is optionally substituted with one to three halogen and is further optionally substituted with one R^{6b1}.

In some embodiments of a compound of Formula I, or a pharmaceutically acceptable salt thereof each R^{6A} is independently 5- to 6-membered heterocyclyl, wherein the heterocyclyl is optionally substituted with one to three halogen and is further substituted with one to three R^{6b1}. In some embodiments of a compound of Formula I, or a pharmaceutically acceptable salt thereof each R^{6A} is independently 5- to 6-membered heterocyclyl, wherein the heterocyclyl is optionally substituted with one to three halogen and is further substituted with one to two R^{6b1}. In some embodiments of a compound of Formula I, or a pharmaceutically acceptable salt thereof each R^{6A} is independently 5- to 6-membered heterocyclyl, wherein the heterocyclyl is optionally substituted with one to three halogen and is further substituted with two R^{6b1}. In some embodiments of a compound of Formula I, or a pharmaceutically acceptable salt thereof each R^{6A} is independently 5- to 6-membered heterocyclyl, wherein the heterocyclyl is optionally substituted with one to three halogen and is further substituted with one R^{6b1}.

In some embodiments of a compound of Formula I, or a pharmaceutically acceptable salt thereof each R^{6A} is independently 5- to 6-membered heteroaryl, wherein the heteroaryl is optionally substituted with one to three halogen and is further optionally substituted with one to three R^{6b1}. In some embodiments of a compound of Formula I, or a pharmaceutically acceptable salt thereof each R^{6A} is independently 5- to 6-membered heteroaryl, wherein the heteroaryl is optionally substituted with one to three halogen and is further optionally substituted with one to two R^{6b1}. In some embodiments of a compound of Formula I, or a pharmaceutically acceptable salt thereof each R^{6A} is independently 5- to 6-membered heteroaryl, wherein the heteroaryl is optionally substituted with one to three halogen and is further optionally substituted with two R^{6b1}. In some embodiments of a compound of Formula I, or a pharmaceutically acceptable salt thereof each R^{6A} is independently 5- to 6-membered heteroaryl, wherein the heteroaryl is optionally substituted with one to three halogen and is further optionally substituted with one R^{6b1}.

In some embodiments of a compound of Formula I, or a pharmaceutically acceptable salt thereof each R^{6A} is independently 5- to 6-membered heteroaryl, wherein the heteroaryl is optionally substituted with one to three halogen and is further substituted with one to three R^{6b1}. In some embodiments of a compound of Formula I, or a pharmaceutically acceptable salt thereof each R^{6A} is independently 5- to 6-membered heteroaryl, wherein the heteroaryl is optionally substituted with one to three halogen and is further substituted with one to two R^{6b1}. In some embodiments of a compound of Formula I, or a pharmaceutically acceptable salt thereof each R^{6A} is independently 5- to 6-membered heteroaryl, wherein the heteroaryl is optionally substituted with one to three halogen and is further substituted with two R^{6b1}.

In some embodiments of a compound of Formula I, or a pharmaceutically acceptable salt thereof each R^{6A} is independently 5- to 6-membered heteroaryl, wherein the heteroaryl is optionally substituted with one to three halogen and is further substituted with one R^{6b1}.

In some embodiments of a compound of Formula I, or a pharmaceutically acceptable salt thereof each R^{6A} is independently —C(O)N(R^{6a1})₂. In some embodiments of a compound of Formula I, or a pharmaceutically acceptable salt thereof at least one R^{6A} is —C(O)N(R^{6a1})₂. In some embodiments of a compound of Formula I, or a pharmaceutically acceptable salt thereof exactly one R^{6A} is —C(O)N(R^{6a1})₂.

In some embodiments of a compound of Formula I, or a pharmaceutically acceptable salt thereof each R^{6A} is independently —N(R^{6a1})C(O)R^{6a1}. In some embodiments of a compound of Formula I, or a pharmaceutically acceptable salt thereof at least one R^{6A} is —N(R^{6a1})C(O)R^{6a1}. In some embodiments of a compound of Formula I, or a pharmaceutically acceptable salt thereof exactly one R^{6A} is —N(R^{6a1})C(O)R^{6a1}.

In some embodiments of a compound of Formula I, or a pharmaceutically acceptable salt thereof each R^{6A} is independently —N(R^{6a1})C(O)OR^{6a1}. In some embodiments of a compound of Formula I, or a pharmaceutically acceptable salt thereof at least one R^{6A} is —N(R^{6a1})C(O)OR^{6a1}. In some embodiments of a compound of Formula I, or a pharmaceutically acceptable salt thereof exactly one R^{6A} is —N(R^{6a1})C(O)OR^{6a1}.

In some embodiments of a compound of Formula I, or a pharmaceutically acceptable salt thereof each R^{6A} is independently —NHC(O)OR^{6a1}. In some embodiments of a compound of Formula I, or a pharmaceutically acceptable salt thereof at least one R^{6A} is —NHC(O)OR^{6a1}. In some embodiments of a compound of Formula I, or a pharmaceutically acceptable salt thereof exactly one R^{6A} is —NHC(O)OR^{6a1}.

In some embodiments of a compound of Formula I, or a pharmaceutically acceptable salt thereof each R^{6A} is independently —S(O)₂R^{6a1}. In some embodiments of a compound of Formula I, or a pharmaceutically acceptable salt thereof at least one R^{6A} is —S(O)₂R^{6a1}. In some embodiments of a compound of Formula I, or a pharmaceutically acceptable salt thereof exactly one R^{6A} is —S(O)₂R^{6a1}.

In some embodiments of a compound of Formula I, or a pharmaceutically acceptable salt thereof each R^{6A} is independently —N═S(O)(R^{6a1})₂. In some embodiments of a compound of Formula I, or a pharmaceutically acceptable salt thereof at least one R^{6A} is —N═S(O)(R^{6a1})₂. In some embodiments of a compound of Formula I, or a pharmaceutically acceptable salt thereof exactly one R^{6A} is —N═S(O)(R^{6a1})₂.

In some embodiments of a compound of Formula I, or a pharmaceutically acceptable salt thereof each R^{6A} is independently —S(NR^{6a1})(O)(R^{6a1}). In some embodiments of a compound of Formula I, or a pharmaceutically acceptable salt thereof at least one R^{6A} is —S(NR^{6a1})(O)(R^{6a1}). In some embodiments of a compound of Formula I, or a pharmaceutically acceptable salt thereof exactly one R^{6A} is —S(NR^{6a1})(O)(R^{6a1}) In some embodiments of a compound of Formula I, or a pharmaceutically acceptable salt thereof each R^{6A} is independently C₁₋₆ alkyl, wherein the alkyl is substituted with one to three R^{6b1}. In some embodiments of a compound of Formula I, or a pharmaceutically acceptable salt thereof at least one R^{6A} is C₁₋₆ alkyl, wherein the alkyl is substituted with one to three R^{6b1}. In some embodiments of a compound of Formula I, or a pharmaceutically acceptable salt thereof exactly one R^{6A} is C₁₋₆ alkyl, wherein the alkyl is substituted with one to three R^{6b1}.

In some embodiments of a compound of Formula I, or a pharmaceutically acceptable salt thereof each R^{6A} is independently C₁₋₆ alkyl, wherein the alkyl is substituted with one to two R^{6b1}. In some embodiments of a compound of Formula I, or a pharmaceutically acceptable salt thereof at least one R^{6A} is C₁₋₆ alkyl, wherein the alkyl is substituted with one to two R^{6b1}. In some embodiments of a compound of Formula I, or a pharmaceutically acceptable salt thereof exactly one R^{6A} is C₁₋₆ alkyl, wherein the alkyl is substituted with one to two R^{6b1}.

In some embodiments of a compound of Formula I, or a pharmaceutically acceptable salt thereof each R^{6A} is independently C₃₋₆ cycloalkyl, wherein the alkyl is substituted with one to three R^{6b1}. In some embodiments of a compound of Formula I, or a pharmaceutically acceptable salt thereof at least one R^{6A} is C₃₋₆ cycloalkyl, wherein the alkyl is substituted with one to three R^{6b1}. In some embodiments of a compound of Formula I, or a pharmaceutically acceptable salt thereof exactly one R^{6A} is C₃₋₆ cycloalkyl, wherein the alkyl is substituted with one to three R^{6b1}.

In some embodiments of a compound of Formula I, or a pharmaceutically acceptable salt thereof each R^{6A} is independently C₃₋₆ cycloalkyl, wherein the alkyl is substituted with one to two R^{6b1}. In some embodiments of a compound of Formula I, or a pharmaceutically acceptable salt thereof at least one R^{6A} is C₃₋₆ cycloalkyl, wherein the alkyl is substituted with one to two R^{6b1}. In some embodiments of a compound of Formula I, or a pharmaceutically acceptable salt thereof exactly one R^{6A} is C₃₋₆ cycloalkyl, wherein the alkyl is substituted with one to two R^{6b1}.

In some embodiments of a compound of Formula I, or a pharmaceutically acceptable salt thereof ring \textcircled{C} is

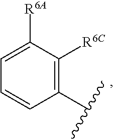

wherein R^{6C} is halogen. In some embodiments of a compound of Formula I, or a pharmaceutically acceptable salt thereof R^{6C} is fluoro. In some embodiments of a compound of Formula I, or a pharmaceutically acceptable salt thereof R^{6C} is chloro.

In some embodiments of a compound of Formula I, II, III, IV, V, VI, or VII, or a pharmaceutically acceptable salt thereof each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl, wherein each alkyl, alkenyl, cycloalkyl, heterocyclyl, aryl, and heteroaryl is optionally substituted with one to three R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached forma 4- to 10-membered heterocyclyl, wherein each alkyl, alkenyl, cycloalkyl, heterocyclyl, aryl, and heteroaryl is optionally substituted with one to two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl, wherein each alkyl, alkenyl, cycloalkyl, heterocyclyl, aryl, and heteroaryl is optionally substituted with two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl, wherein each alkyl, alkenyl, cycloalkyl, heterocyclyl, aryl, and heteroaryl is optionally substituted with one R^{6b1}.

In some embodiments of a compound of Formula I, II, III, IV, V, VI, or VII, or a pharmaceutically acceptable salt thereof each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl, wherein each alkyl, alkenyl, cycloalkyl, heterocyclyl, aryl, and heteroaryl is substituted with one to three R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached forma 4- to 10-membered heterocyclyl, wherein each alkyl, alkenyl, cycloalkyl, heterocyclyl, aryl, and heteroaryl is substituted with one to two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached forma 4- to 10-membered heterocyclyl, wherein each alkyl, alkenyl, cycloalkyl, heterocyclyl, aryl, and heteroaryl is substituted with two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl, wherein each alkyl, alkenyl, cycloalkyl, heterocyclyl, aryl, and heteroaryl is substituted with one R^{6b1}.

In some embodiments of a compound of Formula I, II, III, IV, V, VI, or VII, or a pharmaceutically acceptable salt thereof each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl.

In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, or two R^{6a1} groups taken together with the atom or atoms to which they are attached forma 4- to 10-membered heterocyclyl, wherein each alkyl, cycloalkyl, and heterocyclyl is optionally substituted with one to three R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, or two R^{6a1} groups taken together with the atom or atoms to which they are attached form a 4- to 10-membered heterocyclyl, wherein each alkyl, cycloalkyl, and heterocyclyl is optionally substituted with one to two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, or two R^{6a1} groups taken together with the atom or atoms to which they are attached form a 4- to 10-membered heterocyclyl, wherein each alkyl, cycloalkyl, and heterocyclyl is optionally substituted with two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀cycloalkyl, or 4- to 10-membered heterocyclyl, or two R^{6a1} groups taken together with the atom or atoms to which they are attached form a 4- to 10-membered heterocyclyl, wherein each alkyl, cycloalkyl, and heterocyclyl is optionally substituted with one R^{6b1}.

In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, or two R^{6a1} groups taken together with the atom or atoms to which they are attached forma 4- to 10-membered heterocyclyl, wherein each alkyl, cycloalkyl, and heterocyclyl is substituted with one to three R^{6b1}. In some embodiments of a compound of Formula I, II, III, IV, V, VI, or VII, or a pharmaceutically acceptable salt thereof each R⁶¹ is independently H, C₁₋₆ alkyl, C₃₋₁₀ cycloalkyl, or 4- to 10-membered heterocyclyl, or two R^{6a1} groups taken together with the atom or atoms to which they are attached form a 4- to 10-membered heterocyclyl, wherein each alkyl, cycloalkyl, and heterocyclyl is substituted with one to two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, or two R^{6a1} groups taken together with the atom or atoms to which they are attached form a 4- to 10-membered heterocyclyl, wherein each alkyl, cycloalkyl, and heterocyclyl is substituted with two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, or two R^{6a1} groups taken together with the atom or atoms to which they are attached form a 4- to 10-membered heterocyclyl, wherein each alkyl, cycloalkyl, and heterocyclyl is substituted with one R^{6b1}.

In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, or two R^{6a1} groups taken together with the atom or atoms to which they are attached form a 4- to 10-membered heterocyclyl.

In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with one to three R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with one to two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with one R^{6b1}.

In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, wherein each alkyl, cycloalkyl, and heterocyclyl is substituted with one to three R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, wherein each alkyl, cycloalkyl, and heterocyclyl is substituted with one to two R^{6b1}. In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, wherein each alkyl, cycloalkyl, and heterocyclyl is substituted with two R^{6b1}.

In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl, wherein each alkyl, cycloalkyl, and heterocyclyl is substituted with one R^{6b1}.

In some embodiments of a compound of Formula I, II, III, IV, V, VI, or VII, or a pharmaceutically acceptable salt thereof each R^{6a1} is independently H, C₁₋₆ alkyl, C₃₋₁₀ cycloalkyl, or 4- to 10-membered heterocyclyl.

In some embodiments of a compound of Formula I, II, III, IV, V, VI, or VII, or a pharmaceutically acceptable salt thereof two R^{6a1} groups taken together with the atom or atoms to which they are attached form a 4- to 10-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6a1} groups taken together with the atom or atoms to which they are attached form a 4- to 10-membered heterocyclyl, wherein the heterocyclyl is optionally substituted with one to two R^{6b1}. In some embodiments of a compound of Formula I, II, III, IV, V, VI, or VII, or a pharmaceutically acceptable salt thereof two R^{6a1} groups taken together with the atom or atoms to which they are attached form a 4- to 10-membered heterocyclyl, wherein the heterocyclyl is optionally substituted with two R^{6b1}. In some embodiments of a compound of Formula I, II, III, IV, V, VI, or VII, or a pharmaceutically acceptable salt thereof two R^{6a1} groups taken together with the atom or atoms to which they are attached form a 4- to 10-membered heterocyclyl, wherein the heterocyclyl is optionally substituted with one R^{6b1}.

In some embodiments of a compound of Formula I, II, III, IV, V, VI, or VII, or a pharmaceutically acceptable salt thereof two R^{6a1} groups taken together with the atom or atoms to which they are attached form a 4- to 10-membered heterocyclyl, wherein the heterocyclyl is substituted with one to three R^{6b1}. In some embodiments of a compound of Formula I, II, III, IV, V, VI, or VII, or a pharmaceutically acceptable salt thereof two R^{6a1} groups taken together with the atom or atoms to which they are attached form a 4- to 10-membered heterocyclyl, wherein the heterocyclyl is substituted with one to two R^{6b1}. In some embodiments of a compound of Formula I, II, III, IV, V, VI, or VII, or a pharmaceutically acceptable salt thereof two R^{6a1} groups taken together with the atom or atoms to which they are attached form a 4- to 10-membered heterocyclyl, wherein the heterocyclyl is substituted with two R^{6b1}. In some embodiments of a compound of Formula I, II, III, IV, V, VI, or VII, or a pharmaceutically acceptable salt thereof two R^{6a1} groups taken together with the atom or atoms to which they are attached form a 4- to 10-membered heterocyclyl, wherein the heterocyclyl is substituted with one R^{6b1}.

In some embodiments of a compound of Formula I, II, III, IV, V, VI, or VII, or a pharmaceutically acceptable salt thereof two R^{6a1} groups taken together with the atom or atoms to which they are attached form a 4- to 6-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6a1} groups taken together with the atom or atoms to which they are attached form a 5- to 6-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6a1} groups taken together with the atom or atoms to which they are attached form a 5-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6a1} groups taken together with the atom or atoms to which they are attached form a 6-membered heterocyclyl, wherein the heterocyclyl is optionally substituted with one to three R^{6b1}.

In some embodiments of a compound of Formula I, II, III, IV, V, VI, or VII, or a pharmaceutically acceptable salt thereof two R^{6a1} groups taken together with the atom or atoms to which they are attached form a 4- to 6-membered heterocyclyl. In some embodiments of a compound of Formula I, II, III, IV, V, VI, or VII, or a pharmaceutically acceptable salt thereof two R^{6a1} groups taken together with the atom or atoms to which they are attached form a 5- to 6-membered heterocyclyl. In some embodiments of a compound of Formula I, II, III, IV, V, VI, or VII, or a pharmaceutically acceptable salt thereof two R^{6a1} groups taken together with the atom or atoms to which they are attached form a 5-membered heterocyclyl. In some embodiments of a compound of Formula I, II, III, IV, V, VI, or VII, or a pharmaceutically acceptable salt thereof two R^{6a1} groups taken together with the atom or atoms to which they are attached form a 6-membered heterocyclyl.

In some embodiments of a compound of Formula I, II, III, IV, V, VI, or VII, or a pharmaceutically acceptable salt thereof two R^{6a1} groups taken together with the nitrogen atom to which they are attached form a 4- to 10-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6a1} groups taken together with the nitrogen atom to which they are attached form a 4- to 10-membered heterocyclyl, wherein the heterocyclyl is optionally substituted with one to two R^{6b1}. In some embodiments of a compound of Formula I, II, III, IV, V, VI, or VII, or a pharmaceutically acceptable salt thereof two R^{6a1} groups taken together with the nitrogen atom to which they are attached form a 4- to 10-membered heterocyclyl, wherein the heterocyclyl is optionally substituted with two R^{6b1}. In some embodiments of a compound of Formula I, II, III, IV, V, VI, or VII, or a pharmaceutically acceptable salt thereof two R^{6a1} groups taken together with the nitrogen atom to which they are attached form a 4- to 10-membered heterocyclyl, wherein the heterocyclyl is optionally substituted with one R^{6b1}.

In some embodiments of a compound of Formula I, II, III, IV, V, VI, or VII, or a pharmaceutically acceptable salt thereof two R^{6a1} groups taken together with the nitrogen atom to which they are attached form a 4- to 10-membered heterocyclyl, wherein the heterocyclyl is substituted with one to three R^{6b1}. In some embodiments of a compound of Formula I, II, III, IV, V, VI, or VII, or a pharmaceutically acceptable salt thereof two R^{6a1} groups taken together with the nitrogen atom to which they are attached form a 4- to 10-membered heterocyclyl, wherein the heterocyclyl is substituted with one to two R^{6b1}. In some embodiments of a compound of Formula I, II, III, IV, V, VI, or VII, or a pharmaceutically acceptable salt thereof two R^{6a1} groups taken together with the nitrogen atom to which they are attached form a 4- to 10-membered heterocyclyl, wherein the heterocyclyl is substituted with two R^{6b1}. In some embodiments of a compound of Formula I, II, III, IV, V, VI, or VII, or a pharmaceutically acceptable salt thereof two R^{6a1} groups taken together with the nitrogen atom to which they are attached form a 4- to 10-membered heterocyclyl, wherein the heterocyclyl is substituted with one R^{6b1}.

In some embodiments of a compound of Formula I, II, III, IV, V, VI, or VII, or a pharmaceutically acceptable salt thereof two R^{6a1} groups taken together with the nitrogen atom to which they are attached form a 4- to 6-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6a1} groups taken together with the nitrogen atom to which they are attached form a 5- to 6-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6a1} groups taken together with the nitrogen atom to which they are attached form a 5-membered heterocyclyl, wherein the heterocyclyl is optionally substituted with one to three R^{6b1}. In some embodiments of a compound of Formula I, II, III, IV, V, VI, or VII, or a pharmaceutically acceptable salt thereof two R^{6a1} groups taken together with the nitrogen atom to which they are attached form a 6-membered heterocyclyl, wherein the heterocyclyl is optionally substituted with one to three R^{6b1}.

In some embodiments of a compound of Formula I, II, III, IV, V, VI, or VII, or a pharmaceutically acceptable salt thereof two R^{6a1} groups taken together with the nitrogen atom to which they are attached form a 4- to 6-membered heterocyclyl. In some embodiments of a compound of Formula I, II, III, IV, V, VI, or VII, or a pharmaceutically acceptable salt thereof two R^{6a1} groups taken together with the nitrogen atom to which they are attached form a 5- to 6-membered heterocyclyl. In some embodiments of a compound of Formula I, II, III, IV, V, VI, or VII, or a pharmaceutically acceptable salt thereof two R^{6a1} groups taken together with the nitrogen atom to which they are attached form a 5-membered heterocyclyl. In some embodiments of a compound of Formula I, II, III, IV, V, VI, or VII, or a pharmaceutically acceptable salt thereof two R^{6a1} groups taken together with the nitrogen to which they are attached form a 6-membered heterocyclyl.

In some embodiments of a compound of Formula I, II, III, IV, V, VI, or VII, or a pharmaceutically acceptable salt thereof each R^{6a1} is independently H, C₁₋₆ alkyl, or C₃₋₆ cycloalkyl, wherein each alkyl and cycloalkyl is optionally substituted with one to three R^{6b1}. In some embodiments of a compound of Formula I, II, III, IV, V, VI, or VII, or a pharmaceutically acceptable salt thereof each R^{6a1} is independently H, C₁₋₆ alkyl, or C₃₋₆ cycloalkyl, wherein each alkyl and cycloalkyl is optionally substituted with one to two R^{6b1}. In some embodiments of a compound of Formula I, II, III, IV, V, VI, or VII, or a pharmaceutically acceptable salt thereof each R^{6a1} is independently H, C₁₋₆ alkyl, or C₃₋₆ cycloalkyl, wherein each alkyl and cycloalkyl is optionally substituted with two R^{6b1}. In some embodiments of a compound of Formula I, II, III, IV, V, VI, or VII, or a pharmaceutically acceptable salt thereof each R^{6a1} is independently H, C₁₋₆ alkyl, or C₃₋₆ cycloalkyl, wherein each alkyl and cycloalkyl is optionally substituted with one R^{6b1}.

In some embodiments of a compound of Formula I, II, III, IV, V, VI, or VII, or a pharmaceutically acceptable salt thereof each R^{6a1} is independently H, C₁₋₆ alkyl, or C₃₋₆ cycloalkyl, wherein each alkyl and cycloalkyl is substituted with one to three R^{6b1}. In some embodiments of a compound of Formula I, II, III, IV, V, VI, or VII, or a pharmaceutically acceptable salt thereof each R^{6a1} is independently H, C₁₋₆ alkyl, or C₃₋₆ cycloalkyl, wherein each alkyl and cycloalkyl is substituted with one to two R^{6b1}. In some embodiments of a compound of Formula I, II, III, IV, V, VI, or VII, or a pharmaceutically acceptable salt thereof each R^{6a1} is independently H, C₁₋₆ alkyl, or C₃₋₆ cycloalkyl, wherein each alkyl and cycloalkyl is substituted with two R^{6b1}. In some embodiments of a compound of Formula I, II, III, IV, V, VI, or VII, or a pharmaceutically acceptable salt thereof each R^{6a1} is independently H, C₁₋₆ alkyl, or C₃₋₆ cycloalkyl, wherein each alkyl and cycloalkyl is substituted with one R^{6b1}.

In some embodiments of a compound of Formula I, II, III, IV, V, VI, or VII, or a pharmaceutically acceptable salt thereof each R^{6a1} is independently H, C₁₋₆ alkyl, or C₃₋₆ cycloalkyl. In some embodiments of a compound of Formula I, II, III, IV, V, VI, or VII, or a pharmaceutically acceptable salt thereof each R^{6a1} is independently H, C₁₋₃ alkyl, or C₃₋₆ cycloalkyl.

In some embodiments of a compound of Formula I, II, III, IV, V, VI, or VII, or a pharmaceutically acceptable salt thereof each R^{6a1} is independently H or C₁₋₃ alkyl, wherein each alkyl is optionally substituted with one to three R^{6b1}. In some embodiments of a compound of Formula I, II, III, IV, V, VI, or VII, or a pharmaceutically acceptable salt thereof each R^{6a1} is independently H or C₁₋₃ alkyl, wherein each alkyl is optionally substituted with one to two R^{6b1}. In some embodiments of a compound of Formula I, II, III, IV, V, VI, or VII, or a pharmaceutically acceptable salt thereof each R^{6a1} is independently H or C₁₋₃ alkyl, wherein each alkyl is optionally substituted with two R^{6b1}. In some embodiments of a compound of Formula I, II, III, IV, V, VI, or VII, or a pharmaceutically acceptable salt thereof each R^{6a1} is independently H or C₁₋₃ alkyl, wherein each alkyl is optionally substituted with one R^{6b1}.

In some embodiments of a compound of Formula I, II, III, IV, V, VI, or VII, or a pharmaceutically acceptable salt thereof each R^{6a1} is independently H or C₁₋₃ alkyl, wherein each alkyl is substituted with one to three R^{6b1}. In some embodiments of a compound of Formula I, II, III, IV, V, VI, or VII, or a pharmaceutically acceptable salt thereof each R^{6a1} is independently H or C₁₋₃ alkyl, wherein each alkyl is substituted with one to two R^{6b1}. In some embodiments of a compound of Formula I, II, III, IV, V, VI, or VII, or a pharmaceutically acceptable salt thereof each R^{6a1} is independently H or C₁₋₃ alkyl, wherein each alkyl is substituted with two R^{6b1}. In some embodiments of a compound of Formula I, II, III, IV, V, VI, or VII, or a pharmaceutically acceptable salt thereof each R^{6a1} is independently H or C₁₋₃ alkyl, wherein each alkyl is substituted with one R^{6b1}.

In some embodiments of a compound of Formula I, II, III, IV, V, VI, or VII, or a pharmaceutically acceptable salt thereof each R^{6a1} is independently H or C₁₋₃ alkyl. In some embodiments of a compound of Formula I, II, III, IV, V, VI, or VII, or a pharmaceutically acceptable salt thereof each R^{6a1} is independently H or methyl.

In some embodiments of a compound of Formula I, II, III, IV, V, VI, or VII, or a pharmaceutically acceptable salt thereof at least one R^{6a1} is H.

In some embodiments of a compound of Formula I, II, III, IV, V, VI, or VII, or a pharmaceutically acceptable salt thereof at least one R^{6a1} is C₁₋₃ alkyl. In some embodiments of a compound of Formula I, II, III, IV, V, VI, or VII, or a pharmaceutically acceptable salt thereof at least one R^{6a1} is methyl.

In some embodiments of a compound of Formula I, II, III, IV, V, VI, or VII, or a pharmaceutically acceptable salt thereof each R^{6a1} is independently H or C₃ cycloalkyl, wherein the cycloalkyl is optionally substituted with one to three R^{6b1}. In some embodiments of a compound of Formula I, II, III, IV, V, VI, or VII, or a pharmaceutically acceptable salt thereof each R^{6a1} is independently H or C₃ cycloalkyl, wherein the cycloalkyl is optionally substituted with one to two R^{6b1}.

In some embodiments of a compound of Formula I, II, III, IV, V, VI, or VII, or a pharmaceutically acceptable salt thereof each R^{6a1} is independently H or C₃ cycloalkyl, wherein the cycloalkyl is optionally substituted with two R^{6b1}. In some embodiments of a compound of Formula I, II, III, IV, V, VI, or VII, or a pharmaceutically acceptable salt thereof each R^{6a1} is independently H or C₃ cycloalkyl, wherein the cycloalkyl is optionally substituted with one R^{6b1}.

In some embodiments of a compound of Formula I, II, III, IV, V, VI, or VII, or a pharmaceutically acceptable salt thereof each R^{6a1} is independently H or C₃ cycloalkyl.

In some embodiments of a compound of Formula I, II, III, IV, V, VI, or VII, or a pharmaceutically acceptable salt thereof each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is optionally substituted with one to three R^{6c1}. In some embodiments of a compound of Formula I, II, III, IV, V, VI, or VII, or a pharmaceutically acceptable salt thereof each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is optionally substituted with one to two R^{6c1}. In some embodiments of a compound of Formula I, II, III, IV, V, VI, or VII, or a pharmaceutically acceptable salt thereof each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R¹², —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is optionally substituted with two R^{6c1}. In some embodiments of a compound of Formula I, II, III, IV, V, VI, or VII, or a pharmaceutically acceptable salt thereof each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2}, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR¹²)N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is optionally substituted with one R^{6c1} In some embodiments of a compound of Formula I, II, III, IV, V, VI, or VII, or a pharmaceutically acceptable salt thereof each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R¹²)₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is substituted with one to three R^{6c1} In some embodiments of a compound of Formula I, II, III, IV, V, VI, or VII, or a pharmaceutically acceptable salt thereof each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2}, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is substituted with one to two R^{6c1}. In some embodiments of a compound of Formula I, II, III, IV, V, VI, or VII, or a pharmaceutically acceptable salt thereof each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R²)₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is substituted with two R^{6c1}. In some embodiments of a compound of Formula I, II, III, IV, V, VI, or VII, or a pharmaceutically acceptable salt thereof each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR¹², —C(O)—R^{6b2}, —C(O)O—R¹², —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is substituted with one R^{6c1} In some embodiments of a compound of Formula I, II, III, IV, V, VI, or VII, or a pharmaceutically acceptable salt thereof each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂.

In some embodiments of a compound of Formula I, II, III, IV, V, VI, or VII, or a pharmaceutically acceptable salt thereof each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), —S(O)₂R^{6b2}, or —S(O)₂N(R^{6b2})₂ wherein each alkyl and cycloalkyl is optionally substituted with one to three R^{6c1}. In some embodiments of a compound of Formula I, II, III, IV, V, VI, or VII, or a pharmaceutically acceptable salt thereof each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), —S(O)₂R^{6b2}, or —S(O)₂N(R^{6b2})₂ wherein each alkyl and cycloalkyl is optionally substituted with one to two R^{6c1}. In some embodiments of a compound of Formula I, II, III, IV, V, VI, or VII, or a pharmaceutically acceptable salt thereof each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), —S(O)₂R^{6b2}, or —S(O)₂N(R^{6b2})₂ wherein each alkyl and cycloalkyl is optionally substituted with two R^{6c1}. In some embodiments of a compound of Formula I, II, III, IV, V, VI, or VII, or a pharmaceutically acceptable salt thereof each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), —S(O)₂R^{6b2}, or —S(O)₂N(R^{6b2})₂ wherein each alkyl and cycloalkyl is optionally substituted with one R^{6c1} In some embodiments of a compound of Formula I, II, III, IV, V, VI, or VII, or a pharmaceutically acceptable salt thereof each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), —S(O)₂R¹², or —S(O)₂N(R^{6b2})₂ wherein each alkyl and cycloalkyl is substituted with one to three R^{6c1}. In some embodiments of a compound of Formula I, II, III, IV, V, VI, or VII, or a pharmaceutically acceptable salt thereof each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), —S(O)₂R¹², or —S(O)₂N(R^{6b2})₂ wherein each alkyl and cycloalkyl is substituted with one to two R^{6c1}. In some embodiments of a compound of Formula I, II, III, IV, V, VI, or VII, or a pharmaceutically acceptable salt thereof each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), —S(O)₂R¹², or —S(O)₂N(R^{6b2})₂ wherein each alkyl and cycloalkyl is substituted with two R^{6c1}. In some embodiments of a compound of Formula I, II, III, IV, V, VI, or VII, or a pharmaceutically acceptable salt thereof each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), —S(O)₂R¹², or —S(O)₂N(R^{6b2})₂ wherein each alkyl and cycloalkyl is substituted with one R^{6c1}.

In some embodiments of a compound of Formula I, II, III, IV, V, VI, or VII, or a pharmaceutically acceptable salt thereof each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), —S(O)₂R^{6b2}, or —S(O)₂N(R^{6b2})₂.

In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2}. In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2}, wherein the alkyl is substituted with one to three R^{6c1}. In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2}. In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2}, wherein the alkyl is substituted with one to two R^{6c1}. In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2}. In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2}, wherein the alkyl is substituted with two R^{6c1}. In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2}. In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2}, wherein the alkyl is substituted with one R^{6c1}.

In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2}. In some embodiments of a compound of Formula I, II, III, IV, V, VI, or VII, or a pharmaceutically acceptable salt thereof each R^{6b1} is independently halogen, C₁₋₆ alkyl, —OR^{6b2}, or —S(O)₂R^{6b2} In some embodiments of a compound of Formula I, II, III, IV, V, VI, or VII, or a pharmaceutically acceptable salt thereof each R^{6b1} is independently fluoro, —OH, or methyl.

In some embodiments of a compound of Formula I, II, III, IV, V, VI, or VII, or a pharmaceutically acceptable salt thereof each R^{6b1} is independently halogen. In some embodiments of a compound of Formula I, II, III, IV, V, VI, or VII, or a pharmaceutically acceptable salt thereof at least one R^{6b1} is halogen. In some embodiments of a compound of Formula I, II, III, IV, V, VI, or VII, or a pharmaceutically acceptable salt thereof each R^{6b1} is independently halogen.

In some embodiments of a compound of Formula I, II, III, IV, V, VI, or VII, or a pharmaceutically acceptable salt thereof at least one R^{6b1} is —OH.

In some embodiments of a compound of Formula I, II, III, IV, V, VI, or VII, or a pharmaceutically acceptable salt thereof at least one R^{6b1} is methyl.

In some embodiments of a compound of Formula I, II, III, IV, V, VI, or VII, or a pharmaceutically acceptable salt thereof each R^{6b2} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, wherein each alkyl, alkenyl, cycloalkyl, heterocyclyl, aryl, and heteroaryl is optionally substituted with one to three R^{6c2}.

In some embodiments of a compound of Formula I, II, III, IV, V, VI, or VII, or a pharmaceutically acceptable salt thereof each R^{6b2} is independently H or C₁₋₃ alkyl. In some embodiments of a compound of Formula I, II, III, IV, V, VI, or VII, or a pharmaceutically acceptable salt thereof each R^{6b2} is independently H or methyl.

In some embodiments of a compound of Formula I, II, III, IV, V, VI, or VII, or a pharmaceutically acceptable salt thereof at least one R^{6b2} is H. In some embodiments of a compound of Formula I, II, III, IV, V, VI, or VII, or a pharmaceutically acceptable salt thereof R^{6b2} is H. In some embodiments of a compound of Formula I, II, III, IV, V, VI, or VII, or a pharmaceutically acceptable salt thereof at least one R^{6b2} is methyl. In some embodiments of a compound of Formula I, II, III, IV, V, VI, or VII, or a pharmaceutically acceptable salt thereof R^{6b2} is methyl.

In some embodiments of a compound of Formula I, II, III, IV, V, VI, or VII, or a pharmaceutically acceptable salt thereof each R^{6c1} and R^{6c2} is independently oxo, halogen, —NO₂, —N₃, —CN, —OH, R^{6d1}, —O(R^{6d1}), —OC(O)(R^{6d1}), —NH₂, —NH(R^{6d1}), —N(R^{6d1})₂, —C(O)(R^{6d1}), —C(O)O(R^{6d1}), —C(O)NH₂, —C(O)NH(R^{6d1}), —C(O)N(R^{6d1})₂, —NHC(O)(R^{6d1}), —NHC(O)O(R^{6d1}), —NHC(O)NH(R^{6d1}), —SH, —S(R^{6d1}), —NHS(O)(R^{6d1}), —N(R^{6d1})(S(O)(R^{6d1}), —S(O)N(R^{6d1})₂, —S(O)(R^{6d1}), —S(O)(NH)(R^{6d1}), —S(O)₂(R^{6d1}), —S(O)₂NH(R^{6d1}), or —S(O)₂N(R^{6d1})₂.

In some embodiments of a compound of Formula I, II, III, IV, V, VI, or VII, or a pharmaceutically acceptable salt thereof each R^{6c1} and R^{6c2} is independently halogen, —OH, or —NH₂. In some embodiments of a compound of Formula I, II, III, IV, V, VI, or VII, or a pharmaceutically acceptable salt thereof each R^{6c1} and R^{6c2} is independently halogen. In some embodiments of a compound of Formula I, II, III, IV, V, VI, or VII, or a pharmaceutically acceptable salt thereof each R^{6c1} and R^{6c2} is —OH. In some embodiments of a compound of Formula I, II, III, IV, V, VI, or VII, or a pharmaceutically acceptable salt thereof each R^{6c1} and R^{6c2} is —NH₂.

In some embodiments of a compound of Formula I, II, III, IV, V, VI, or VII, or a pharmaceutically acceptable salt thereof each R^{6d1} is independently C₁₋₆ alkyl, C₃₋₁₀ cycloalkyl, 4- to 10-membered heterocyclyl, C₆₋₁₀ aryl, or 5- to 10-membered heteroaryl, wherein each alkyl, alkenyl, alkynyl, cycloalkyl, heterocyclyl aryl, and heteroaryl is optionally substituted with one to three R^{6e1} In some embodiments of a compound of Formula I, II, III, IV, V, VI, or VII, or a pharmaceutically acceptable salt thereof each R^{6d1} is independently C₁₋₆ alkyl, C₃₋₁₀ cycloalkyl, 4- to 10-membered heterocyclyl, C₆₋₁₀ aryl, or 5- to 10-membered heteroaryl.

In some embodiments of a compound of Formula I, II, III, IV, V, VI, or VII, or a pharmaceutically acceptable salt thereof each R^{6d1} is independently C₁₋₆ alkyl. In some embodiments of a compound of Formula I, II, III, IV, V, VI, or VII, or a pharmaceutically acceptable salt thereof each R^{6d1} is independently C₁₋₃ alkyl.

In some embodiments of a compound of Formula I, II, III, IV, V, VI, or VII, or a pharmaceutically acceptable salt thereof each R^{6e1} is independently oxo, halogen, —NO₂, —N₃, —CN, —OH, —NH₂, methyl, or halomethyl.

In some embodiments of a compound of Formula I, II, III, IV, V, VI, or VII, or a pharmaceutically acceptable salt thereof each heterocyclyl and heteroaryl independently has one to four ring heteroatoms each independently selected from N, O, and S. In some embodiments of a compound of Formula I, II, III, IV, V, VI, or VII, or a pharmaceutically acceptable salt thereof each heterocyclyl and heteroaryl independently has one to four ring heteroatoms each independently selected from N and O. In some embodiments of a compound of Formula I, II, III, IV, V, VI, or VII, or a pharmaceutically acceptable salt thereof each heterocyclyl and heteroaryl independently has one to four ring heteroatoms that are N.

In some embodiments of a compound of Formula I, II, III, IV, V, VI, or VII, or a pharmaceutically acceptable salt thereof each heterocyclyl and heteroaryl independently has one to three ring heteroatoms each independently selected from N, O, and S. In some embodiments of a compound of Formula I, II, III, IV, V, VI, or VII, or a pharmaceutically acceptable salt thereof each heterocyclyl and heteroaryl independently has one to three ring heteroatoms each independently selected from N and O. In some embodiments of a compound of Formula I, II, III, IV, V, VI, or VII, or a pharmaceutically acceptable salt thereof each heterocyclyl and heteroaryl independently has one to three ring heteroatoms that are N.

In some embodiments of a compound of Formula I, II, III, IV, V, VI, or VII, or a pharmaceutically acceptable salt thereof each heterocyclyl and heteroaryl independently has one to two ring heteroatoms each independently selected from N, O, and S. In some embodiments of a compound of Formula I, II, III, IV, V, VI, or VII, or a pharmaceutically acceptable salt thereof each heterocyclyl and heteroaryl independently has one to two ring heteroatoms each independently selected from N and O. In some embodiments of a compound of Formula I, II, III, IV, V, VI, or VII, or a pharmaceutically acceptable salt thereof each heterocyclyl and heteroaryl independently has one to two ring heteroatoms that are N.

In some embodiments of a compound of Formula I, II, III, IV, V, VI, or VII, or a pharmaceutically acceptable salt thereof each heterocyclyl and heteroaryl independently has two ring heteroatoms each independently selected from N, O, and S. In some embodiments of a compound of Formula I, II, III, IV, V, VI, or VII, or a pharmaceutically acceptable salt thereof each heterocyclyl and heteroaryl independently has two ring heteroatoms each independently selected from N and O. In some embodiments of a compound of Formula I, II, III, IV, V, VI, or VII, or a pharmaceutically acceptable salt thereof each heterocyclyl and heteroaryl independently has two ring heteroatoms that are N.

In some embodiments of a compound of Formula I, II, III, IV, V, VI, or VII, or a pharmaceutically acceptable salt thereof each heterocyclyl and heteroaryl independently has one ring heteroatom selected from N, O, and S. In some embodiments of a compound of Formula I, II, III, IV, V, VI, or VII, or a pharmaceutically acceptable salt thereof each heterocyclyl and heteroaryl independently has one ring heteroatom selected from N and O. In some embodiments of a compound of Formula I, II, III, IV, V, VI, or VII, or a pharmaceutically acceptable salt thereof each heterocyclyl and heteroaryl independently has one ring heteroatom that is N.

In one embodiment, provided herein is a compound selected from Table I:

TABLE I

| Ex. No. | Structure | Notes |
|---|---|---|
| 1.1 | 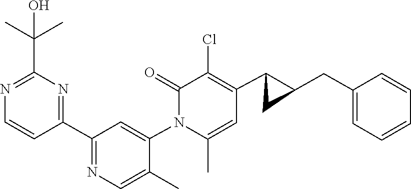 [SMILES: Cc1cnc(-c2ccnc(C(C)(C)O)n2)cc1-n1c(C)cc([C@H]2C[C@@H]2Cc2ccccc2)c(Cl)c1=O] | Mixture of atropisomers, single known cyclopropyl stereoisomer |
| 1.2 | 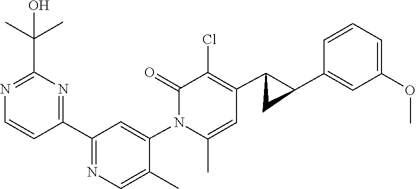 [SMILES: COc1cccc([C@H]2C[C@@H]2c2cc(C)n(-c3cc(-c4ccnc(C(C)(C)O)n4)ncc3C)c(=O)c2Cl)c1] | Mixture of atropisomers, single known cyclopropyl stereoisomer |
| 1.3 | 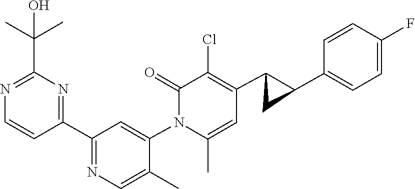 [SMILES: Cc1cnc(-c2ccnc(C(C)(C)O)n2)cc1-n1c(C)cc([C@H]2C[C@@H]2c2ccc(F)cc2)c(Cl)c1=O] | Mixture of atropisomers, single known cyclopropyl stereoisomer |
| 1.4 | 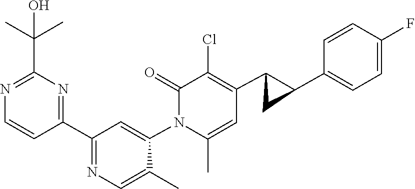 | Single known stereoisomer |
| 1.5 | 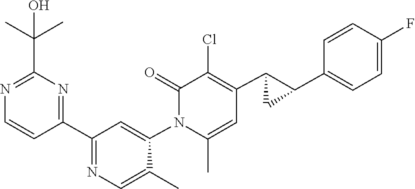 | Single known stereoisomer |
| 1.6 | 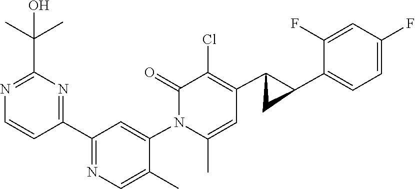 [SMILES: Cc1cnc(-c2ccnc(C(C)(C)O)n2)cc1-n1c(C)cc([C@H]2C[C@@H]2c2ccc(F)cc2F)c(Cl)c1=O] | Mixture of atropisomers, single known cyclopropyl stereoisomer |
| 1.7 | 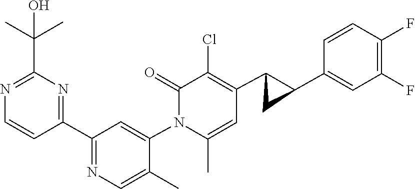 [SMILES: Cc1cnc(-c2ccnc(C(C)(C)O)n2)cc1-n1c(C)cc([C@H]2C[C@@H]2c2ccc(F)c(F)c2)c(Cl)c1=O] | Mixture of atropisomers, single known cyclopropyl stereoisomer |
| 1.8 | 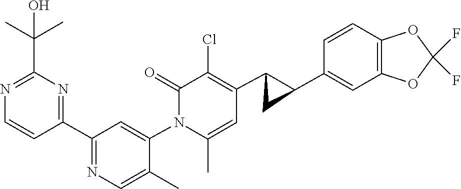 [SMILES: Cc1cnc(-c2ccnc(C(C)(C)O)n2)cc1-n1c(C)cc([C@H]2C[C@@H]2c2ccc3c(c2)OC(F)(F)O3)c(Cl)c1=O] | Mixture of atropisomers, single known cyclopropyl stereoisomer |
| 1.9 | 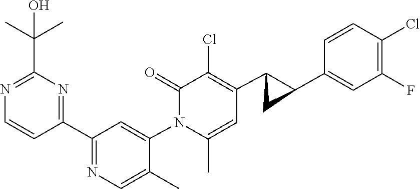 [SMILES: Cc1cnc(-c2ccnc(C(C)(C)O)n2)cc1-n1c(C)cc([C@H]2C[C@@H]2c2ccc(Cl)c(F)c2)c(Cl)c1=O] | Mixture of atropisomers, single known cyclopropyl stereoisomer |
| 1.10 | 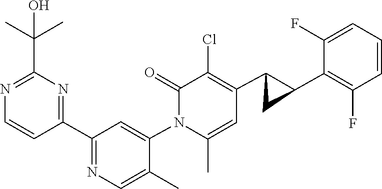 [SMILES: Cc1cnc(-c2ccnc(C(C)(C)O)n2)cc1-n1c(C)cc([C@H]2C[C@@H]2c2c(F)cccc2F)c(Cl)c1=O] | Mixture of atropisomers, single known cyclopropyl stereoisomer |
| 1.11 | 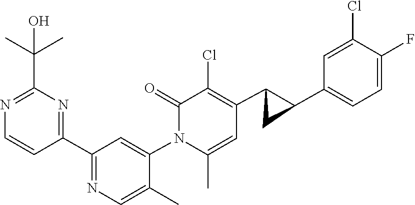 [SMILES: Cc1cnc(-c2ccnc(C(C)(C)O)n2)cc1-n1c(C)cc([C@H]2C[C@@H]2c2ccc(F)c(Cl)c2)c(Cl)c1=O] | Mixture of atropisomers, single known cyclopropyl stereoisomer |

| | | |
|---|---|---|
| 1.12 | 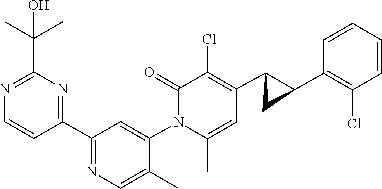 [SMILES: Cc1cnc(-c2ccnc(C(C)(C)O)n2)cc1-n1c(C)cc([C@H]2C[C@@H]2c2ccccc2Cl)c(Cl)c1=O] | Mixture of atropisomers, single known cyclopropyl stereoisomer |

| | | |
|---|---|---|
| 1.13 | 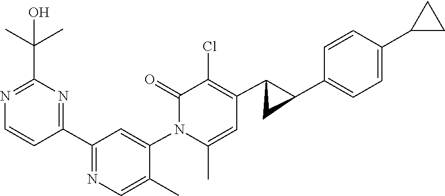 [SMILES: Cc1cnc(-c2ccnc(C(C)(C)O)n2)cc1-n1c(C)cc([C@H]2C[C@@H]2c2ccc(C3CC3)cc2)c(Cl)c1=O] | Mixture of atropisomers, single known cyclopropyl stereoisomer |

| | | |
|---|---|---|
| 1.14 | 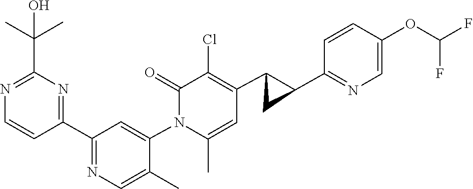 [SMILES: Cc1cnc(-c2ccnc(C(C)(C)O)n2)cc1-n1c(C)cc([C@H]2C[C@@H]2c2ccc(OC(F)F)cn2)c(Cl)c1=O] | Mixture of atropisomers, single known cyclopropyl stereoisomer |

| | | |
|---|---|---|
| 1.15 | 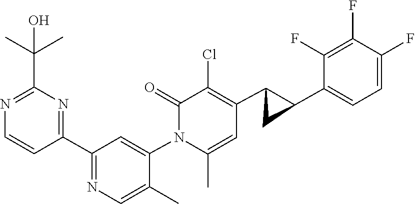 [SMILES: Cc1cnc(-c2ccnc(C(C)(C)O)n2)cc1-n1c(C)cc([C@H]2C[C@@H]2c2ccc(F)c(F)c2F)c(Cl)c1=O] | Mixture of atropisomers, single known cyclopropyl stereoisomer |

| | | |
|---|---|---|
| 1.16 | 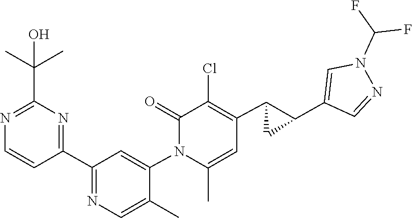 [SMILES: Cc1cnc(-c2ccnc(C(C)(C)O)n2)cc1-n1c(C)cc([C@@H]2C[C@H]2c2cnn(C(F)F)c2)c(Cl)c1=O] | Mixture of atropisomers, single known cyclopropyl stereoisomer |

| | | |
|---|---|---|
| 1.17 | 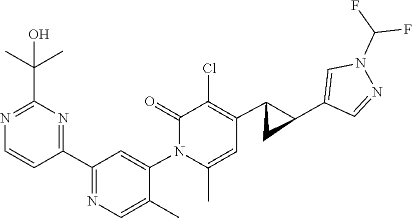 [SMILES: Cc1cnc(-c2ccnc(C(C)(C)O)n2)cc1-n1c(C)cc([C@H]2C[C@@H]2c2cnn(C(F)F)c2)c(Cl)c1=O] | Mixture of atropisomers, single known cyclopropyl stereoisomer |

| | | |
|---|---|---|
| 1.18 | 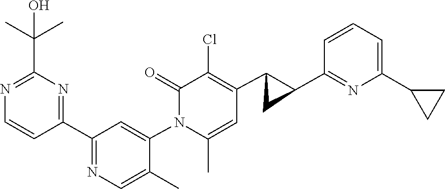 [SMILES: Cc1cnc(-c2ccnc(C(C)(C)O)n2)cc1-n1c(C)cc([C@H]2C[C@@H]2c2cccc(C3CC3)n2)c(Cl)c1=O] | Mixture of atropisomers, single known cyclopropyl stereoisomer |

| | | |
|---|---|---|
| 1.19 | 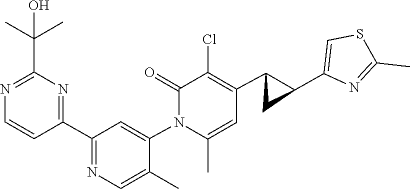 [SMILES: Cc1nc([C@H]2C[C@@H]2c2cc(C)n(-c3cc(-c4ccnc(C(C)(C)O)n4)ncc3C)c(=O)c2Cl)cs1] | Mixture of atropisomers, single known cyclopropyl stereoisomer |

| | | |
|---|---|---|
| 1.20 | 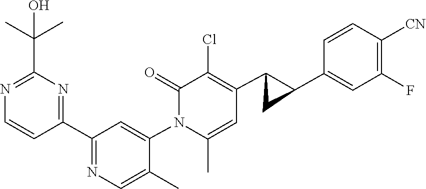 [SMILES: Cc1cnc(-c2ccnc(C(C)(C)O)n2)cc1-n1c(C)cc([C@H]2C[C@@H]2c2ccc(C#N)c(F)c2)c(Cl)c1=O] | Mixture of atropisomers, single known cyclopropyl stereoisomer |

| | | |
|---|---|---|
| 1.21 | 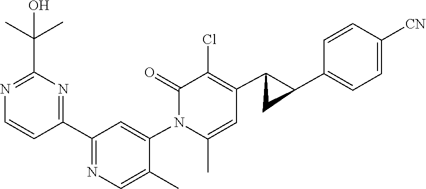 [SMILES: Cc1cnc(-c2ccnc(C(C)(C)O)n2)cc1-n1c(C)cc([C@H]2C[C@@H]2c2ccc(C#N)cc2)c(Cl)c1=O] | Mixture of atropisomers, single known cyclopropyl stereoisomer |

| | | |
|---|---|---|
| 1.22 | 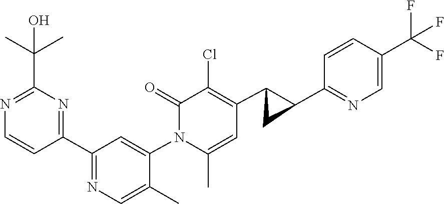 [SMILES: Cc1cnc(-c2ccnc(C(C)(C)O)n2)cc1-n1c(C)cc([C@H]2C[C@@H]2c2ccc(C(F)(F)F)cn2)c(Cl)c1=O] | Mixture of atropisomers, single known cyclopropyl stereoisomer |

| | | |
|---|---|---|
| 1.23 | 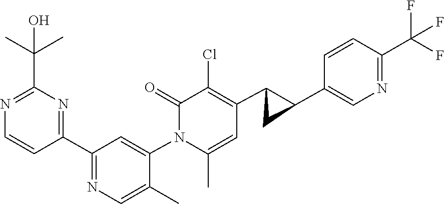 [SMILES: Cc1cnc(-c2ccnc(C(C)(C)O)n2)cc1-n1c(C)cc([C@H]2C[C@@H]2c2ccc(C(F)(F)F)nc2)c(Cl)c1=O] | Mixture of atropisomers, single known cyclopropyl stereoisomer |

| | | |
|---|---|---|
| 1.24 | 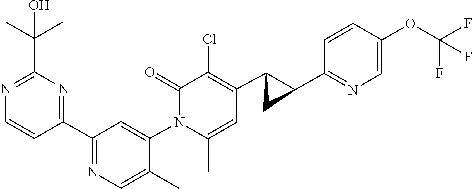 [SMILES: Cc1cnc(-c2ccnc(C(C)(C)O)n2)cc1-n1c(C)cc([C@H]2C[C@@H]2c2ccc(OC(F)(F)F)cn2)c(Cl)c1=O] | Mixture of atropisomers, single known cyclopropyl stereoisomer |

| | | |
|---|---|---|
| 1.25 | 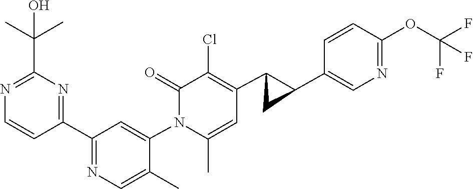 [SMILES: Cc1cnc(-c2ccnc(C(C)(C)O)n2)cc1-n1c(C)cc([C@H]2C[C@@H]2c2ccc(OC(F)(F)F)nc2)c(Cl)c1=O] | Mixture of atropisomers, single known cyclopropyl stereoisomer |

| | | |
|---|---|---|
| 1.26 | 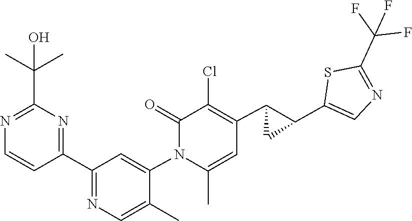 [SMILES: Cc1cnc(-c2ccnc(C(C)(C)O)n2)cc1-n1c(C)cc([C@@H]2C[C@H]2c2cnc(C(F)(F)F)s2)c(Cl)c1=O] | Mixture of atropisomers, single known cyclopropyl stereoisomer |

| | | |
|---|---|---|
| 1.27 | 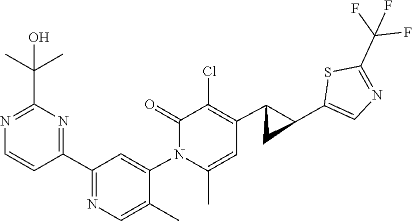 [SMILES: Cc1cnc(-c2ccnc(C(C)(C)O)n2)cc1-n1c(C)cc([C@H]2C[C@@H]2c2cnc(C(F)(F)F)s2)c(Cl)c1=O] | Mixture of atropisomers, single known cyclopropyl stereoisomer |

| | | |
|---|---|---|
| 1.28 | 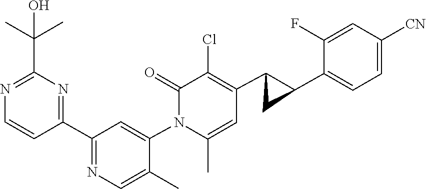 [SMILES: Cc1cnc(-c2ccnc(C(C)(C)O)n2)cc1-n1c(C)cc([C@H]2C[C@@H]2c2ccc(C#N)cc2F)c(Cl)c1=O] | Mixture of atropisomers, single known cyclopropyl stereoisomer |

| | | |
|---|---|---|
| 1.29 | 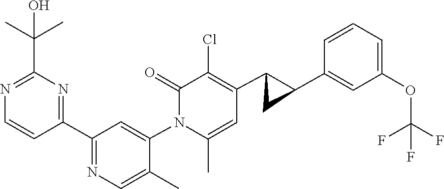 [SMILES: Cc1cnc(-c2ccnc(C(C)(C)O)n2)cc1-n1c(C)cc([C@H]2C[C@@H]2c2cccc(OC(F)(F)F)c2)c(Cl)c1=O] | Mixture of atropisomers, single known cyclopropyl stereoisomer |

| | | |
|---|---|---|
| 1.30 | 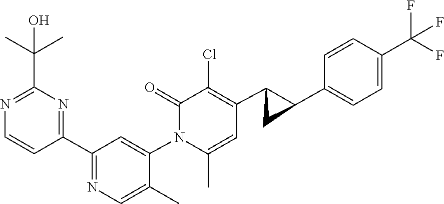 [SMILES: Cc1cnc(-c2ccnc(C(C)(C)O)n2)cc1-n1c(C)cc([C@H]2C[C@@H]2c2ccc(C(F)(F)F)cc2)c(Cl)c1=O] | Mixture of atropisomers, single known cyclopropyl stereoisomer |

| | | |
|---|---|---|
| 1.31 | 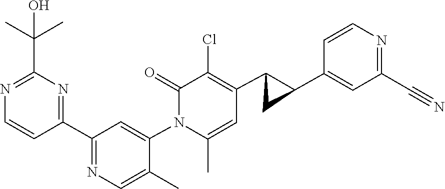 [SMILES: Cc1cnc(-c2ccnc(C(C)(C)O)n2)cc1-n1c(C)cc([C@H]2C[C@@H]2c2ccnc(C#N)c2)c(Cl)c1=O] | Mixture of atropisomers, single known cyclopropyl stereoisomer |

| | | |
|---|---|---|
| 1.32 | 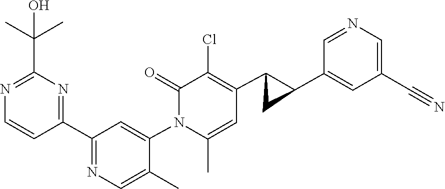 [SMILES: Cc1cnc(-c2ccnc(C(C)(C)O)n2)cc1-n1c(C)cc([C@H]2C[C@@H]2c2cncc(C#N)c2)c(Cl)c1=O] | Mixture of atropisomers, single known cyclopropyl stereoisomer |

| | | |
|---|---|---|
| 1.33 | 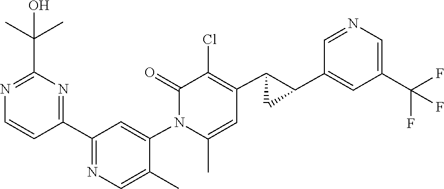 [SMILES: Cc1cnc(-c2ccnc(C(C)(C)O)n2)cc1-n1c(C)cc([C@@H]2C[C@H]2c2cncc(C(F)(F)F)c2)c(Cl)c1=O] | Mixture of atropisomers, single known cyclopropyl stereoisomer |

| | | |
|---|---|---|
| 1.34 | 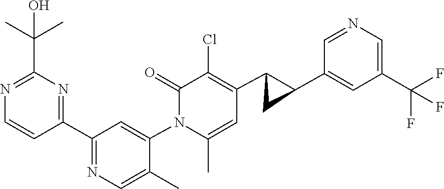 [SMILES: Cc1cnc(-c2ccnc(C(C)(C)O)n2)cc1-n1c(C)cc([C@H]2C[C@@H]2c2cncc(C(F)(F)F)c2)c(Cl)c1=O] | Mixture of atropisomers, single known cyclopropyl stereoisomer |

| | | |
|---|---|---|
| 1.35 | 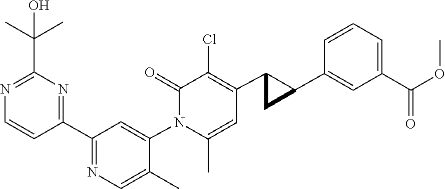 [SMILES: COC(=O)c1cccc([C@H]2C[C@@H]2c2cc(C)n(-c3cc(-c4ccnc(C(C)(C)O)n4)ncc3C)c(=O)c2Cl)c1] | Mixture of atropisomers, mixture of trans cyclopropyl stereoisomers |

| | | |
|---|---|---|
| 1.36 | 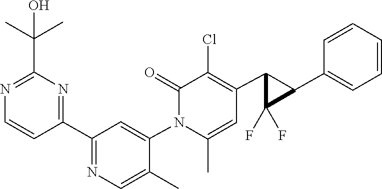 [SMILES: Cc1cnc(-c2ccnc(C(C)(C)O)n2)cc1-n1c(C)cc([C@@H]2[C@@H](c3ccccc3)C2(F)F)c(Cl)c1=O] | Mixture of atropisomers, mixture of trans cyclopropyl stereoisomers |

| | | |
|---|---|---|
| 1.37 | 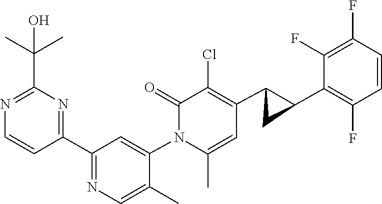 [SMILES: Cc1cnc(-c2ccnc(C(C)(C)O)n2)cc1-n1c(C)cc([C@H]2C[C@@H]2c2c(F)ccc(F)c2F)c(Cl)c1=O] | Mixture of atropisomers, single known cyclopropyl stereoisomer |

| | | |
|---|---|---|
| 1.38 | 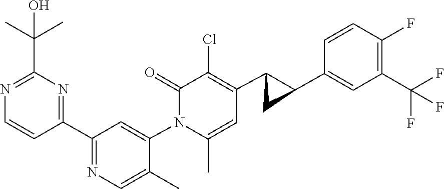 [SMILES: Cc1cnc(-c2ccnc(C(C)(C)O)n2)cc1-n1c(C)cc([C@H]2C[C@@H]2c2ccc(F)c(C(F)(F)F)c2)c(Cl)c1=O] | Mixture of atropisomers, single known cyclopropyl stereoisomer |

| | | |
|---|---|---|
| 1.39 | 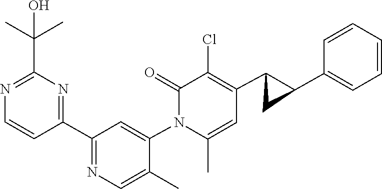 [SMILES: Cc1cnc(-c2ccnc(C(C)(C)O)n2)cc1-n1c(C)cc([C@H]2C[C@@H]2c2ccccc2)c(Cl)c1=O] | Mixture of atropisomers, single known cyclopropyl stereoisomer |

| | | |
|---|---|---|
| 1.40 | 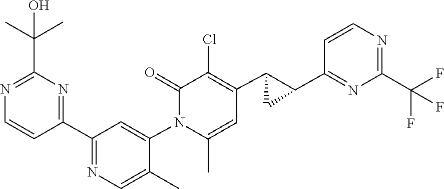 [SMILES: Cc1cnc(-c2ccnc(C(C)(C)O)n2)cc1-n1c(C)cc([C@@H]2C[C@H]2c2ccnc(C(F)(F)F)n2)c(Cl)c1=O] | Mixture of atropisomers, single known cyclopropyl stereoisomer |

| | | |
|---|---|---|
| 1.41 | 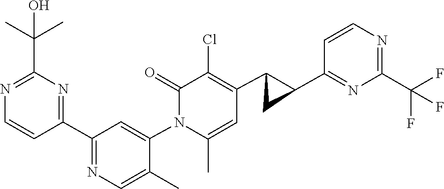 [SMILES: Cc1cnc(-c2ccnc(C(C)(C)O)n2)cc1-n1c(C)cc([C@H]2C[C@@H]2c2ccnc(C(F)(F)F)n2)c(Cl)c1=O] | Mixture of atropisomers, single known cyclopropyl stereoisomer |

| | | |
|---|---|---|
| 1.42 | 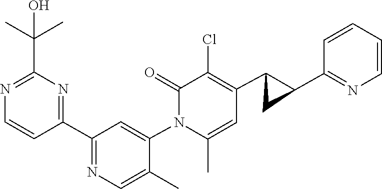 [SMILES: Cc1cnc(-c2ccnc(C(C)(C)O)n2)cc1-n1c(C)cc([C@H]2C[C@@H]2c2ccccn2)c(Cl)c1=O] | Mixture of atropisomers, single known cyclopropyl stereoisomer |

| | | |
|---|---|---|
| 1.43 | 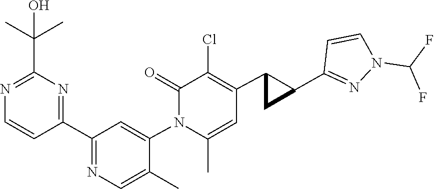 [SMILES: Cc1cnc(-c2ccnc(C(C)(C)O)n2)cc1-n1c(C)cc([C@H]2C[C@@H]2c2ccn(C(F)F)n2)c(Cl)c1=O] | Mixture of atropisomers, mixture of trans cyclopropyl stereoisomers |

| | | |
|---|---|---|
| 1.44 | 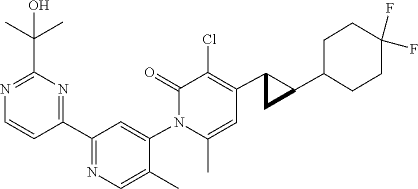 [SMILES: Cc1cnc(-c2ccnc(C(C)(C)O)n2)cc1-n1c(C)cc([C@H]2C[C@@H]2C2CCC(F)(F)CC2)c(Cl)c1=O] | Mixture of atropisomers, mixture of trans cyclopropyl stereoisomers |

| | | |
|---|---|---|
| 1.45 | 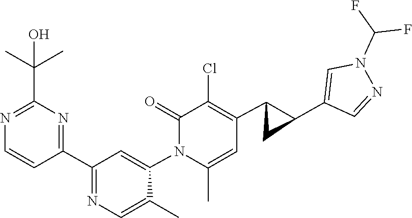 | Single known stereoisomer |

| | | |
|---|---|---|
| 1.46 | 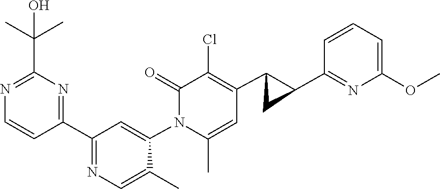 | Single known stereoisomer |

| | | |
|---|---|---|
| 1.47 | 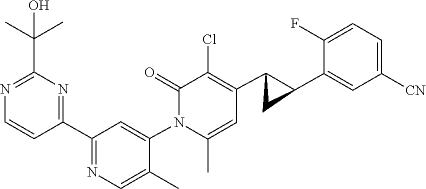 [SMILES: Cc1cnc(-c2ccnc(C(C)(C)O)n2)cc1-n1c(C)cc([C@H]2C[C@@H]2c2cc(C#N)ccc2F)c(Cl)c1=O] | Mixture of atropisomers, single known cyclopropyl stereoisomer |

| | | |
|---|---|---|
| 1.48 | 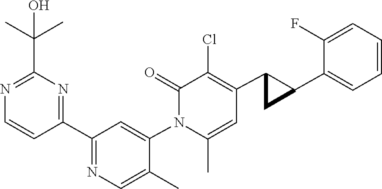 [SMILES: Cc1cnc(-c2ccnc(C(C)(C)O)n2)cc1-n1c(C)cc([C@H]2C[C@@H]2c2ccccc2F)c(Cl)c1=O] | Mixture of atropisomers, mixture of trans cyclopropyl stereoisomers |

| | | |
|---|---|---|
| 1.49 | 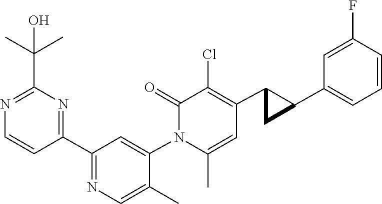 [SMILES: Cc1cnc(-c2ccnc(C(C)(C)O)n2)cc1-n1c(C)cc([C@H]2C[C@@H]2c2cccc(F)c2)c(Cl)c1=O] | Mixture of atropisomers, mixture of trans cyclopropyl stereoisomers |

| | | |
|---|---|---|
| 1.50 | 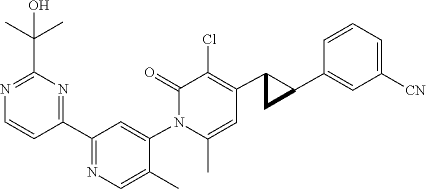 [SMILES: Cc1cnc(-c2ccnc(C(C)(C)O)n2)cc1-n1c(C)cc([C@H]2C[C@@H]2c2cccc(C#N)c2)c(Cl)c1=O] | Mixture of atropisomers, mixture of trans cyclopropyl stereoisomers |

| | | |
|---|---|---|
| 1.51 | 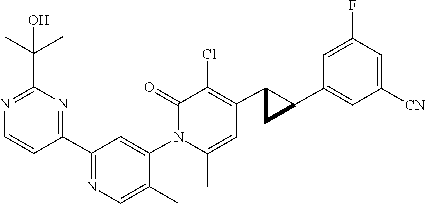 [SMILES: Cc1cnc(-c2ccnc(C(C)(C)O)n2)cc1-n1c(C)cc([C@H]2C[C@@H]2c2cc(F)cc(C#N)c2)c(Cl)c1=O] | Mixture of atropisomers, mixture of trans cyclopropyl stereoisomers |

| | | |
|---|---|---|
| 1.52 | 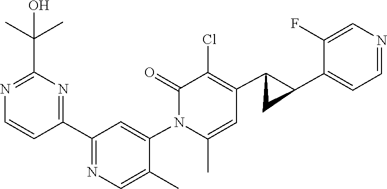 [SMILES: Cc1cnc(-c2ccnc(C(C)(C)O)n2)cc1-n1c(C)cc([C@H]2C[C@@H]2c2ccncc2F)c(Cl)c1=O] | Mixture of atropisomers, single known cyclopropyl stereoisomer |

| | | |
|---|---|---|
| 1.53 | 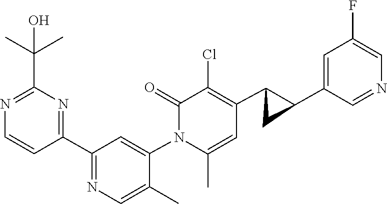 [SMILES: Cc1cnc(-c2ccnc(C(C)(C)O)n2)cc1-n1c(C)cc([C@H]2C[C@@H]2c2cncc(F)c2)c(Cl)c1=O] | Mixture of atropisomers, single known cyclopropyl stereoisomer |

| | | |
|---|---|---|
| 1.54 | 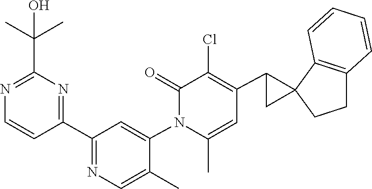 [SMILES: Cc1cnc(-c2ccnc(C(C)(C)O)n2)cc1-n1c(C)cc(C2CC23CCc2ccccc23)c(Cl)c1=O] | Mixture of atropisomers, mixture of cyclopropyl isomers |

| | | |
|---|---|---|
| 1.55 | 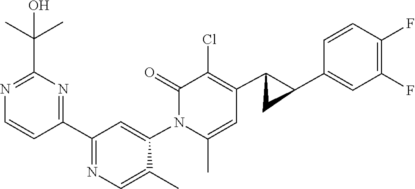 | Single known stereoisomer |

| | | |
|---|---|---|
| 1.56 | 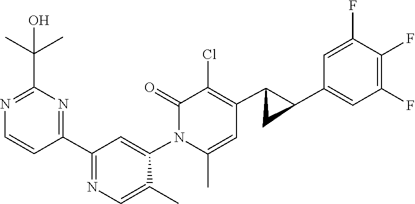 | Single known stereoisomer |

| | | |
|---|---|---|
| 1.57 | 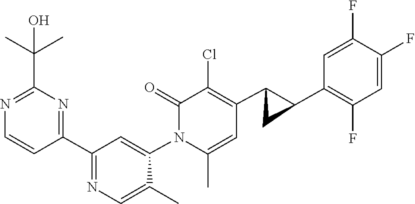 | Single known stereoisomer |

| | | |
|---|---|---|
| 1.58 | 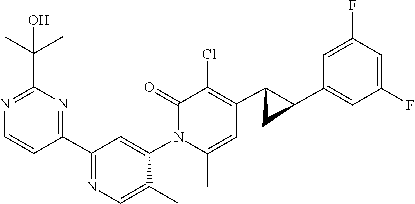 | Single known stereoisomer |

| | | |
|---|---|---|
| 1.59 | 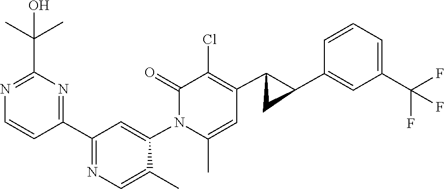 | Single known stereoisomer |

| | | |
|---|---|---|
| 1.60 | 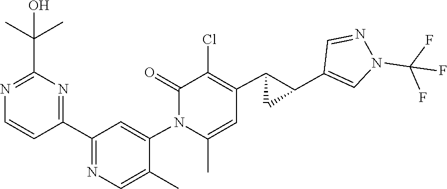 [SMILES: Cc1cnc(-c2ccnc(C(C)(C)O)n2)cc1-n1c(C)cc([C@@H]2C[C@H]2c2cnn(C(F)(F)F)c2)c(Cl)c1=O] | Single known stereoisomer |

| | | |
|---|---|---|
| 1.61 | 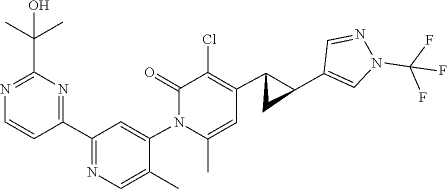 [SMILES: Cc1cnc(-c2ccnc(C(C)(C)O)n2)cc1-n1c(C)cc([C@H]2C[C@@H]2c2cnn(C(F)(F)F)c2)c(Cl)c1=O] | Single known stereoisomer |

| | | |
|---|---|---|
| 1.62 | 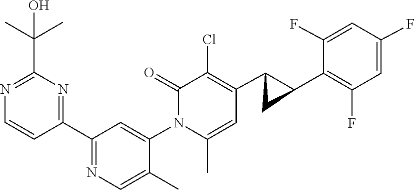 [SMILES: Cc1cnc(-c2ccnc(C(C)(C)O)n2)cc1-n1c(C)cc([C@H]2C[C@@H]2c2c(F)cc(F)cc2F)c(Cl)c1=O] | Single known stereoisomer |

| | | |
|---|---|---|
| 1.63 | 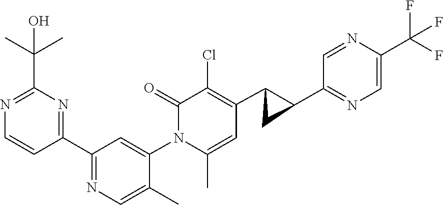 [SMILES: Cc1cnc(-c2ccnc(C(C)(C)O)n2)cc1-n1c(C)cc([C@H]2C[C@@H]2c2cnc(C(F)(F)F)cn2)c(Cl)c1=O] | Single known stereoisomer |

| | | |
|---|---|---|
| 1.64 | 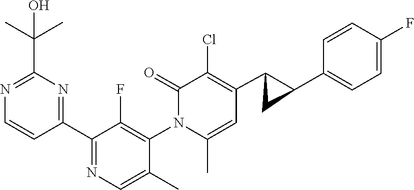 [SMILES: Cc1cnc(-c2ccnc(C(C)(C)O)n2)c(F)c1-n1c(C)cc([C@H]2C[C@@H]2c2ccc(F)cc2)c(Cl)c1=O] | Single known stereoisomer |

| | | |
|---|---|---|
| 1.65 | 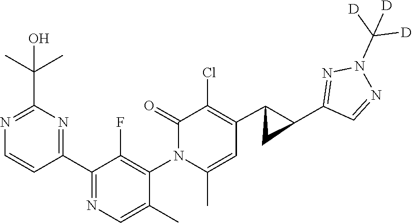 [SMILES: [2H]C([2H])([2H])n1ncc([C@H]2C[C@@H]2c2cc(C)n(-c3c(C)cnc(-c4ccnc(C(C)(C)O)n4)c3F)c(=O)c2Cl)n1] | Single known stereoisomer |

| | | |
|---|---|---|
| 2.1 | 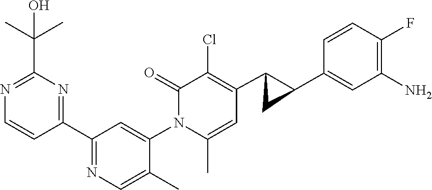 [SMILES: Cc1cnc(-c2ccnc(C(C)(C)O)n2)cc1-n1c(C)cc([C@H]2C[C@@H]2c2ccc(F)c(N)c2)c(Cl)c1=O] | Mixture of atropisomers, single known cyclopropyl stereoisomer |

| | | |
|---|---|---|
| 2.2 | 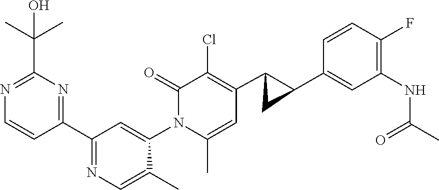 | Single known stereoisomer |

| | | |
|---|---|---|
| 2.3 | 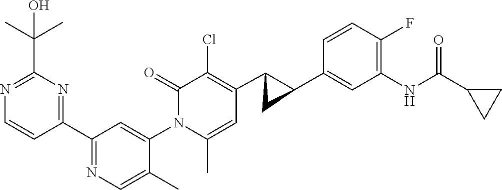 [SMILES: Cc1cnc(-c2ccnc(C(C)(C)O)n2)cc1-n1c(C)cc([C@H]2C[C@@H]2c2ccc(F)c(NC(=O)C3CC3)c2)c(Cl)c1=O] | Mixture of atropisomers, single known cyclopropyl stereoisomer |

| | | |
|---|---|---|
| 3.1 | 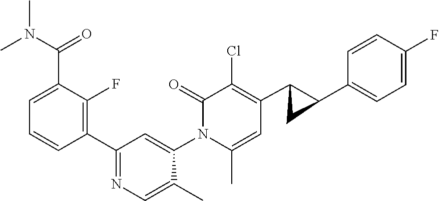 | Single known stereoisomer |

| | | |
|---|---|---|
| 3.2 | 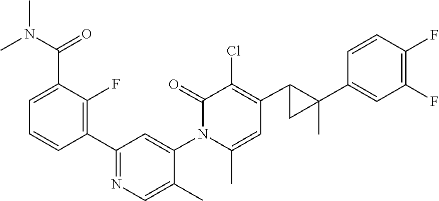 [SMILES: Cc1cnc(-c2cccc(C(=O)N(C)C)c2F)cc1-n1c(C)cc(C2CC2(C)c2ccc(F)c(F)c2)c(Cl)c1=O] | Mixture of atropisomers, mixture of cyclopropyl isomers |

| | | |
|---|---|---|
| 3.3 | 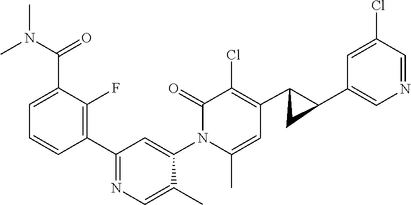 | Single known stereoisomer |

| | | |
|---|---|---|
| 3.4 | 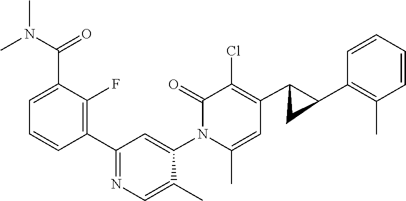 | Single known stereoisomer |

| | | |
|---|---|---|
| 3.5 | 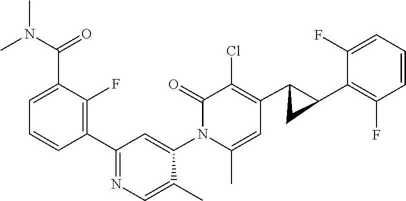 | Single known stereoisomer |

| | | |
|---|---|---|
| 3.6 | 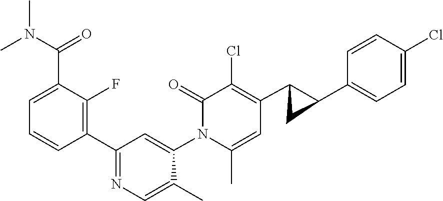 | Single known stereoisomer |

| | | |
|---|---|---|
| 3.7 | 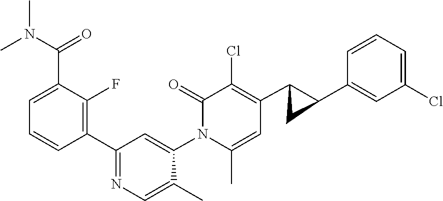 | Single known stereoisomer |

| | | |
|---|---|---|
| 3.8 | 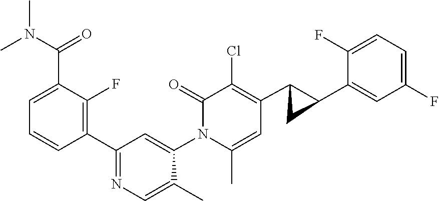 | Single known stereoisomer |

| | | |
|---|---|---|
| 3.9 | 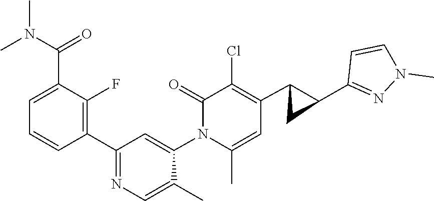 | Single known stereoisomer |

| | | |
|---|---|---|
| 3.10 | 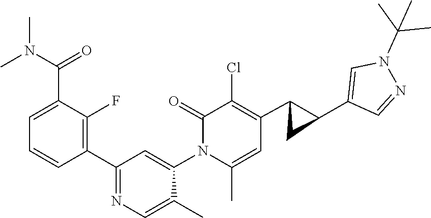 | Single known stereoisomer |

| | | |
|---|---|---|
| 3.11 | 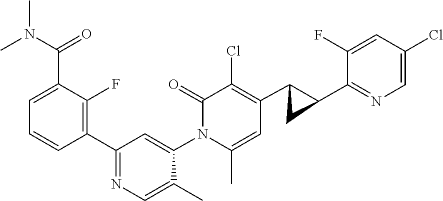 | Single known stereoisomer |

| | | |
|---|---|---|
| 3.12 | 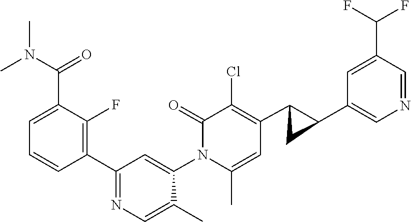 | Single known stereoisomer |

| | | |
|---|---|---|
| 3.13 | 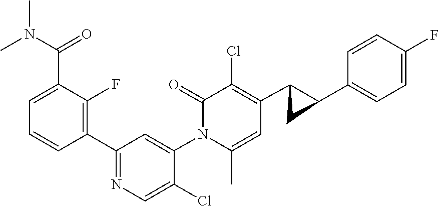 [SMILES: Cc1cc([C@H]2C[C@@H]2c2ccc(F)cc2)c(Cl)c(=O)n1-c1cc(-c2cccc(C(=O)N(C)C)c2F)ncc1Cl] | Mixture of atropisomers, single known cyclopropyl stereoisomer |

| | | |
|---|---|---|
| 3.14 | 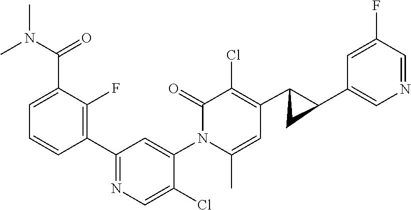 [SMILES: Cc1cc([C@H]2C[C@@H]2c2cncc(F)c2)c(Cl)c(=O)n1-c1cc(-c2cccc(C(=O)N(C)C)c2F)ncc1Cl] | Mixture of atropisomers, single known cyclopropyl stereoisomer |

| | | |
|---|---|---|
| 3.15 | 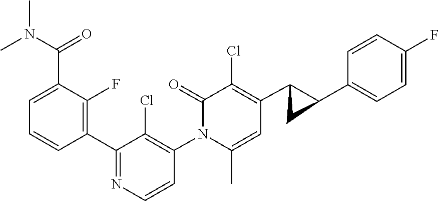 [SMILES: Cc1cc([C@H]2C[C@@H]2c2ccc(F)cc2)c(Cl)c(=O)n1-c1ccnc(-c2cccc(C(=O)N(C)C)c2F)c1Cl] | Mixture of atropisomers, single known cyclopropyl stereoisomer |

| | | |
|---|---|---|
| 3.16 | 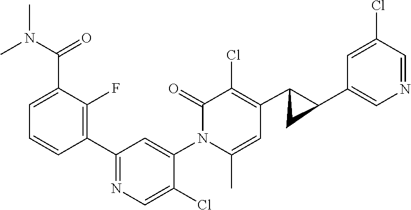 [SMILES: Cc1cc([C@H]2C[C@@H]2c2cncc(Cl)c2)c(Cl)c(=O)n1-c1cc(-c2cccc(C(=O)N(C)C)c2F)ncc1Cl] | Mixture of atropisomers, single known cyclopropyl stereoisomer |

| | | |
|---|---|---|
| 3.17 | 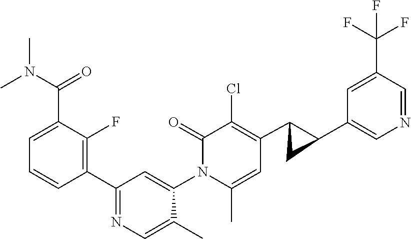 | Single known stereoisomer |

| | | |
|---|---|---|
| 3.18 | 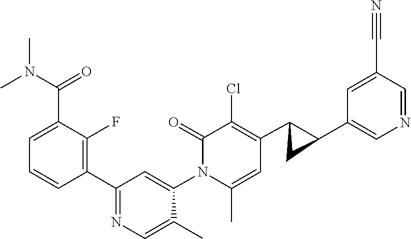 | Single known stereoisomer |

| | | |
|---|---|---|
| 3.19 | 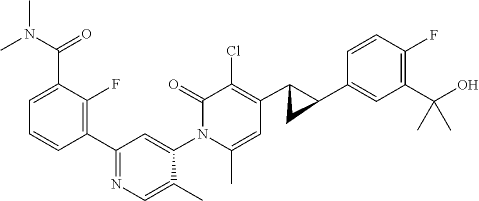 | Single known stereoisomer |

| | | |
|---|---|---|
| 3.20 | 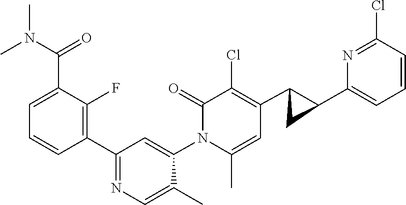 | Single known stereoisomer |

| | | |
|---|---|---|
| 3.21 | 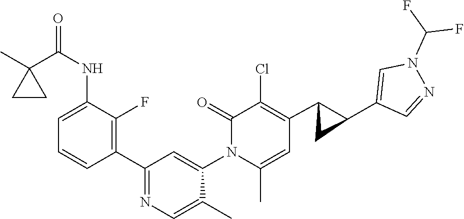 | Single known stereoisomer |

| | | |
|---|---|---|
| 3.22 | 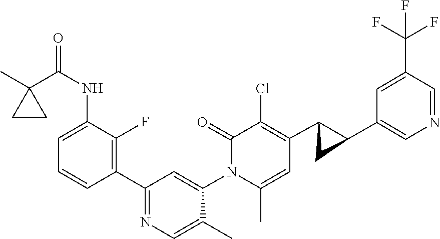 | Single known stereoisomer |

| | | |
|---|---|---|
| 3.23 | 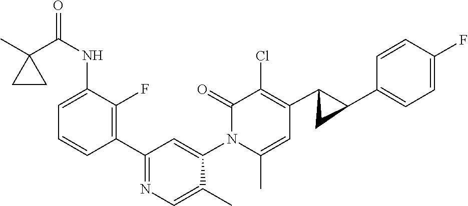 | Single known stereoisomer |

| | | |
|---|---|---|
| 3.24 | 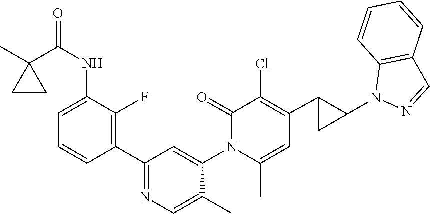 | Single known atropisomer, mixture of cyclopropyl stereoisomers |

| | | |
|---|---|---|
| 3.25 | 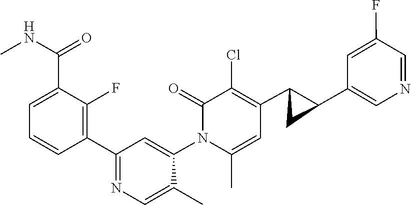 | Single known stereoisomer |

| | | |
|---|---|---|
| 3.26 | 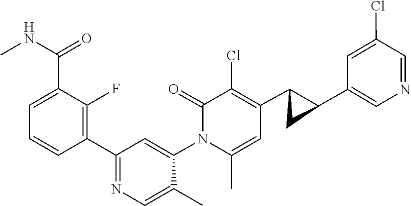 | Single known stereoisomer |

| | | |
|---|---|---|
| 3.27 | 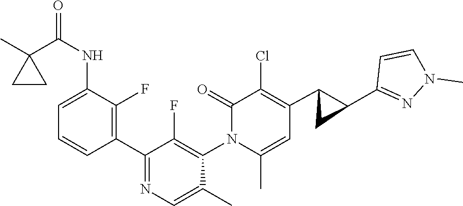 | Single known stereoisomer |

| | | |
|---|---|---|
| 3.28 | 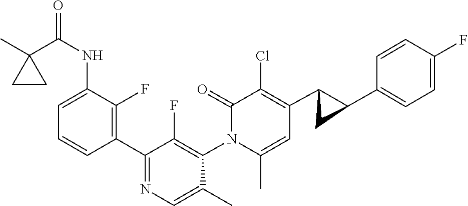 | Single known stereoisomer |

| | | |
|---|---|---|
| 3.29 | 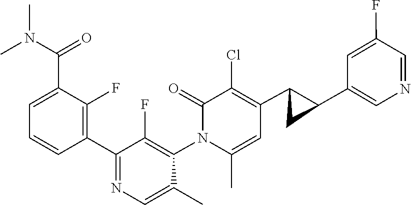 | Single known stereoisomer |

| | | |
|---|---|---|
| 3.30 | 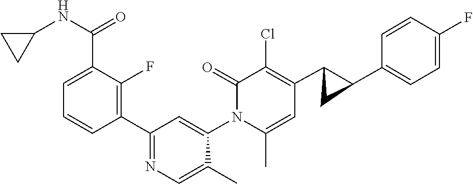 | Single known stereoisomer |

| | | |
|---|---|---|
| 3.31 | 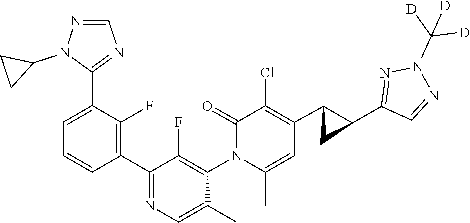 | Single known stereoisomer |

| | | |
|---|---|---|
| 3.32 | 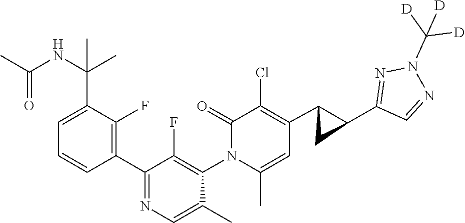 | Single known stereoisomer |

| | | |
|---|---|---|
| 3.33 | 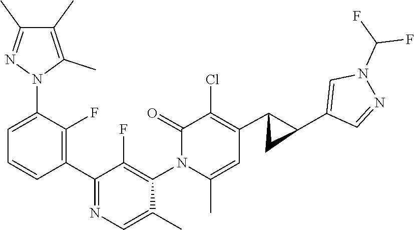 | Single known stereoisomer |

| | | |
|---|---|---|
| 3.34 | 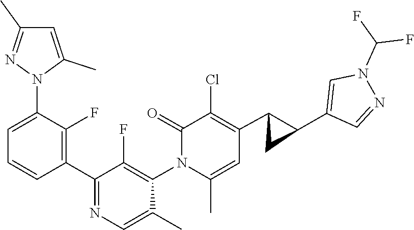 | Single known stereoisomer |

| | | |
|---|---|---|
| 3.35 | 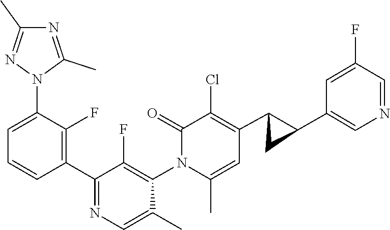 | Single known stereoisomer |

| | | |
|---|---|---|
| 3.36 | 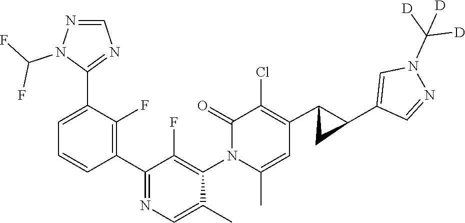 | Single known stereoisomer |

| | | |
|---|---|---|
| 3.37 | 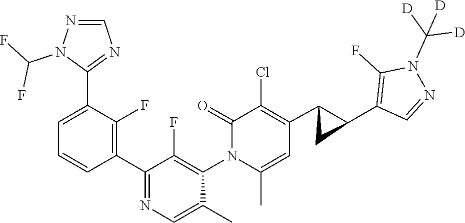 | Single known stereoisomer |

| | | |
|---|---|---|
| 3.38 | 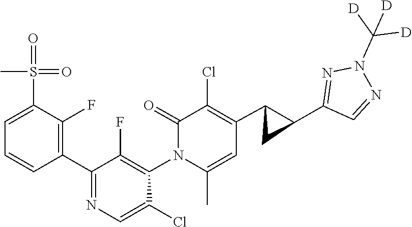 | Single known stereoisomer |

| | | |
|---|---|---|
| 3.39 | 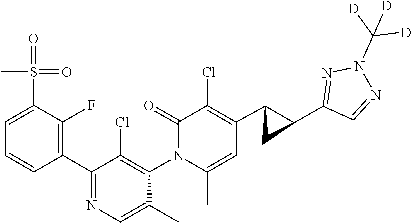 | Single known stereoisomer |

| | | |
|---|---|---|
| 4.1 | 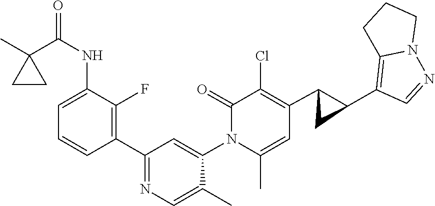 | Single known stereoisomer |

| | | |
|---|---|---|
| 4.2 | 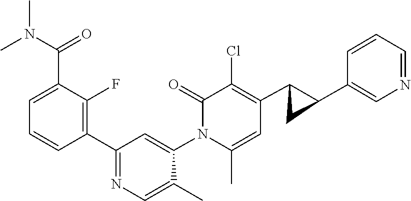 | Single known stereoisomer |

| | | |
|---|---|---|
| 4.3 | 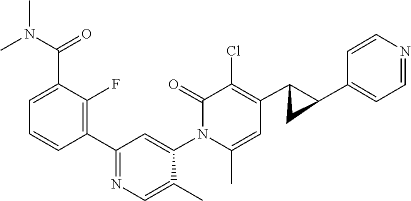 | Single known stereoisomer |

| | | |
|---|---|---|
| 4.4 | 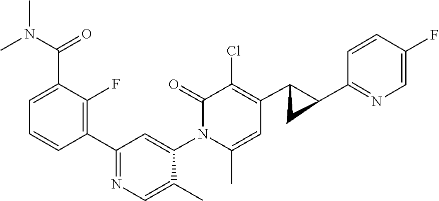 | Single known stereoisomer |
| 4.5 | 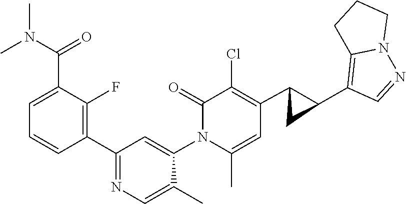 | Single known stereoisomer |
| 4.6 | 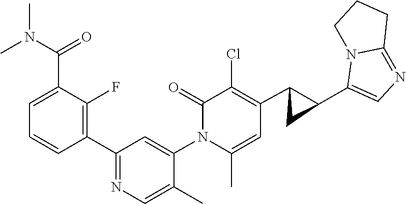 [SMILES: Cc1cnc(-c2cccc(C(=O)N(C)C)c2F)cc1-n1c(C)cc([C@H]2C[C@@H]2c2cnc3n2CCC3)c(Cl)c1=O] | Mixture of atropisomers, single known cyclopropyl stereoisomer |
| 4.7 | 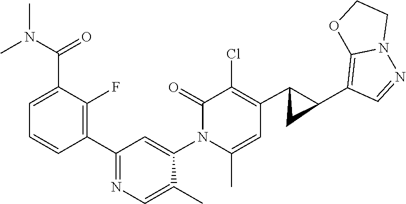 | Single known stereoisomer |
| 4.8 | 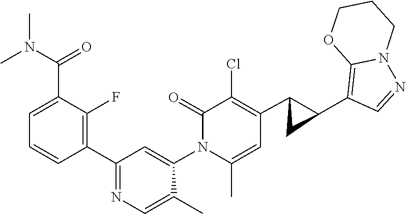 | Single known stereoisomer |
| 4.9 | 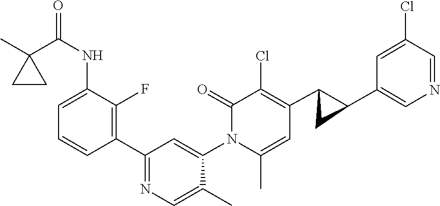 | Single known stereoisomer |
| 4.10 | 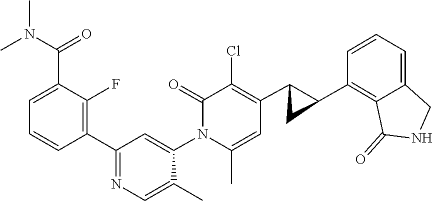 | Single known stereoisomer |
| 4.11 | 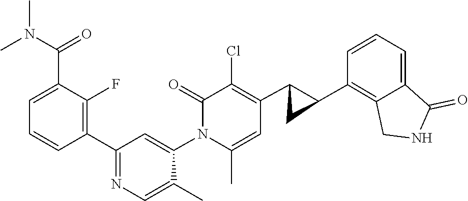 | Single known stereoisomer |
| 4.12 | 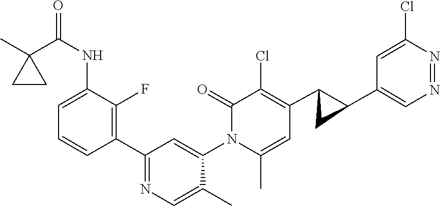 | Single known stereoisomer |
| 4.13 | 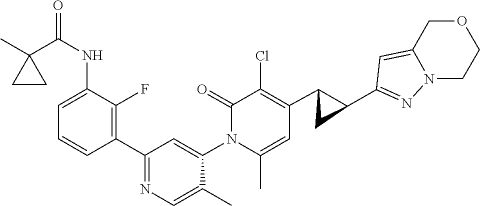 | Single known stereoisomer |
| 4.14 | 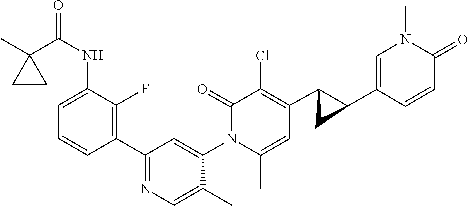 | Single known stereoisomer |
| 4.15 | 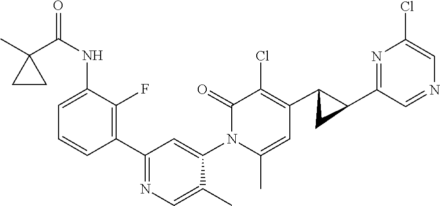 | Single known stereoisomer |
| 4.16 | 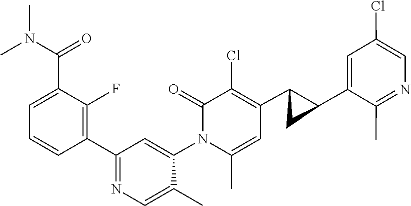 | Single known stereoisomer |
| 4.17 | 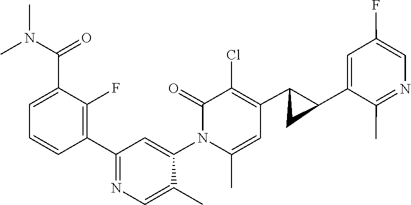 | Single known stereoisomer |
| 4.18 | 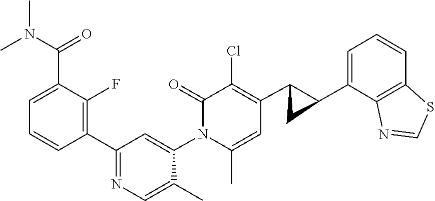 | Single known stereoisomer |
| 4.19 | 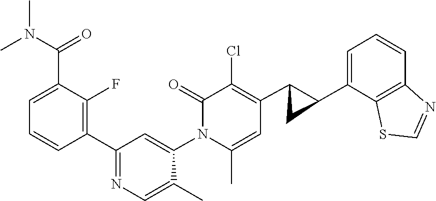 | Single known stereoisomer |
| 4.20 | 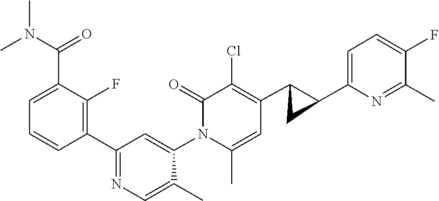 | Single known stereoisomer |
| 4.21 | 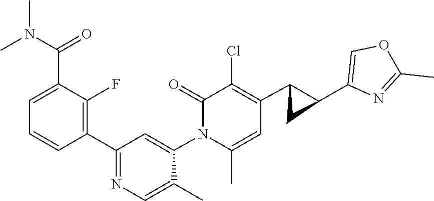 | Single known stereoisomer |
| 4.22 | 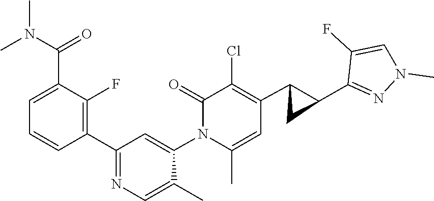 | Single known stereoisomer |
| 4.23 | 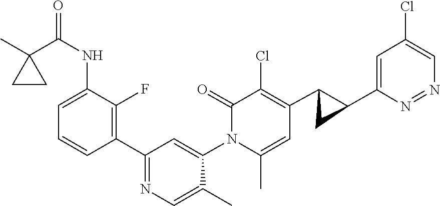 | Single known stereoisomer |
| 4.24 | 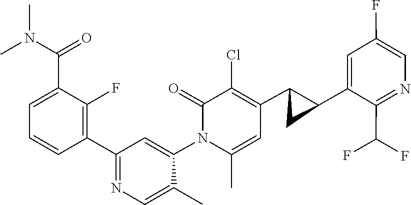 | Single known stereoisomer |
| 4.25 | 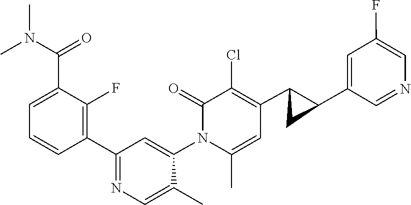 | Single known stereoisomer |
| 4.26 | 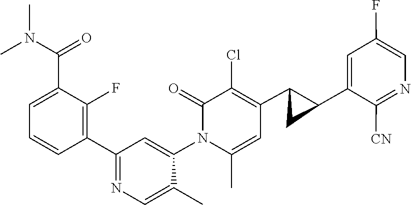 | Single known stereoisomer |
| 4.27 | 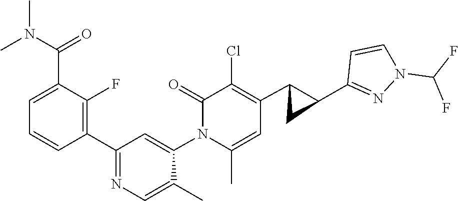 | Single known stereoisomer |
| 4.28 | 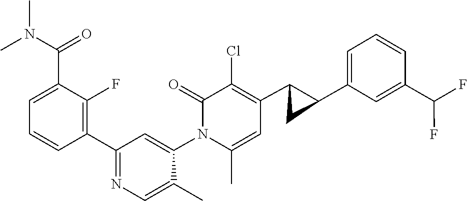 | Single known stereoisomer |
| 4.29 | 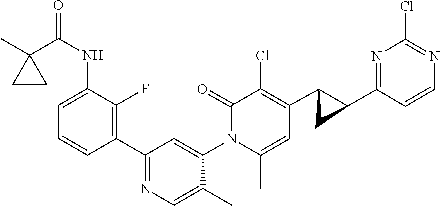 | Single known stereoisomer |
| 4.30 | 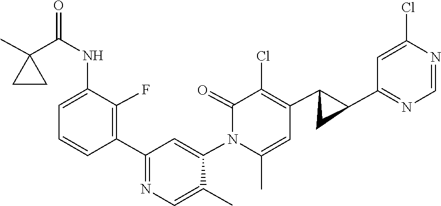 | Single known stereoisomer |
| 4.31 | 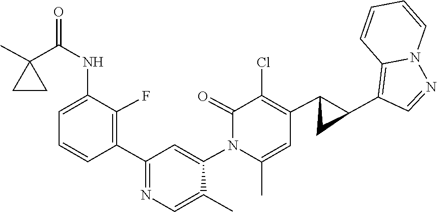 | Single known stereoisomer |
| 4.32 | 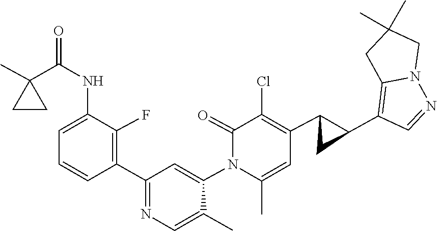 | Single known stereoisomer |
| 4.33 | 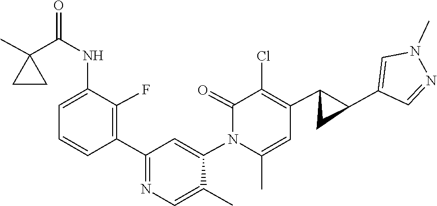 | Single known stereoisomer |
| 4.34 | 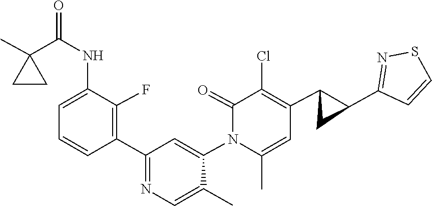 | Single known stereoisomer |
| 4.35 | 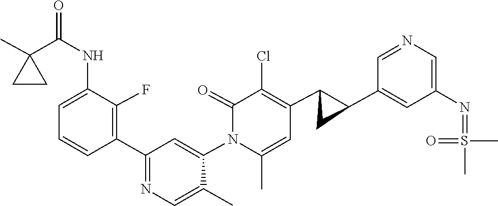 | Single known stereoisomer |
| 4.36 | 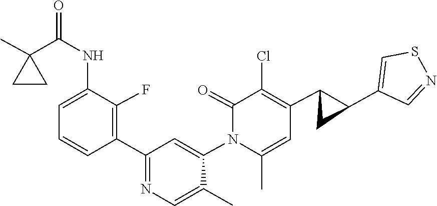 | Single known stereoisomer |
| 4.37 | 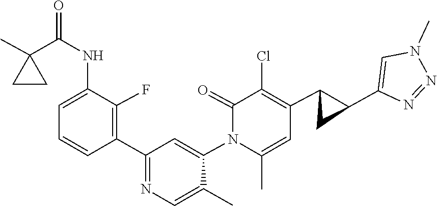 | Single known stereoisomer |
| 4.38 | 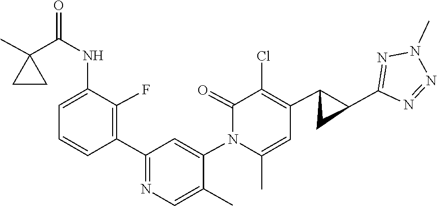 [SMILES: Cc1cnc(-c2cccc(NC(=O)C3(C)CC3)c2F)cc1-n1c(C)cc([C@H]2C[C@@H]2c2nnn(C)n2)c(Cl)c1=O] | Single known stereoisomer |
| 4.39 | 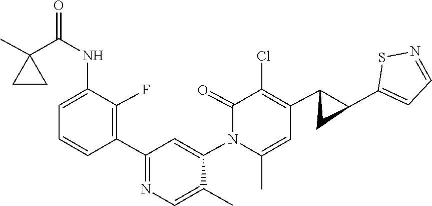 | Single known stereoisomer |
| 4.40 | 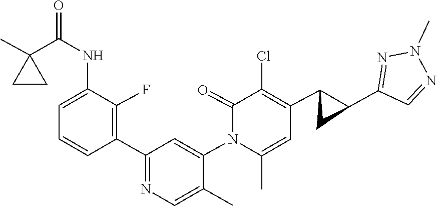 [SMILES: Cc1cnc(-c2cccc(NC(=O)C3(C)CC3)c2F)cc1-n1c(C)cc([C@H]2C[C@@H]2c2cnn(C)n2)c(Cl)c1=O] | Mixture of atropisomers, single known cyclopropyl stereoisomer |
| 4.41 | 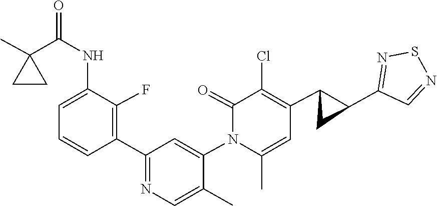 [SMILES: Cc1cnc(-c2cccc(NC(=O)C3(C)CC3)c2F)cc1-n1c(C)cc([C@H]2C[C@@H]2c2cnsn2)c(Cl)c1=O] | Mixture of atropisomers, single known cyclopropyl stereoisomer |
| 4.42 | 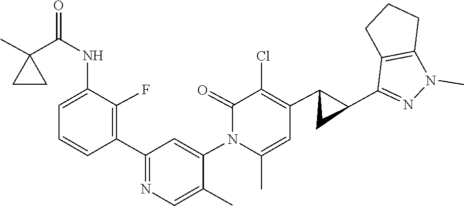 | Mixture of atropisomers, single known cyclopropyl stereoisomer |
| 4.43 | 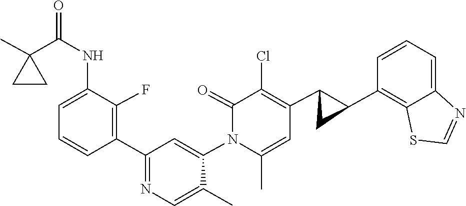 | Single known stereoisomer |
| 4.44 | 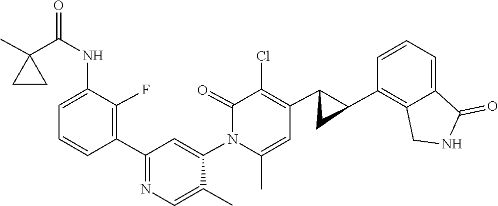 | Single known stereoisomer |
| 4.45 | 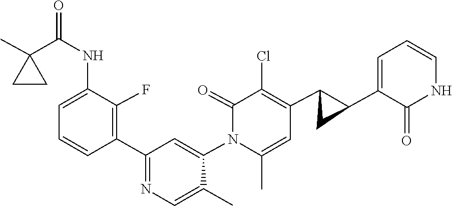 | Single known stereoisomer |
| 4.46 | 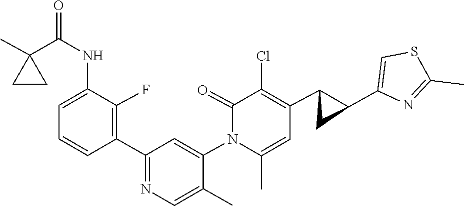 | Mixture of atropisomers, single known cyclopropyl stereoisomer |
| 4.47 | 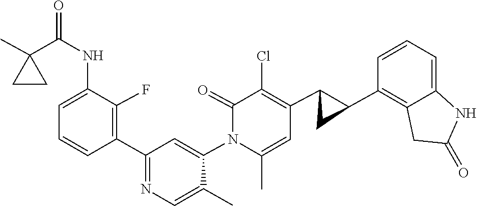 | Single known stereoisomer |
| 4.48 | 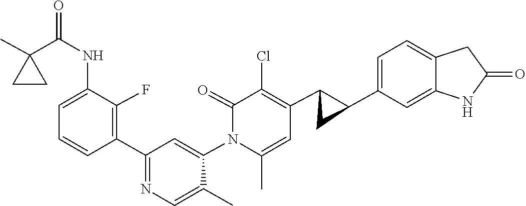 | Single known stereoisomer |
| 4.49 | 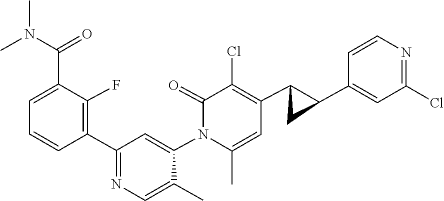 | Single known stereoisomer |
| 4.50 | 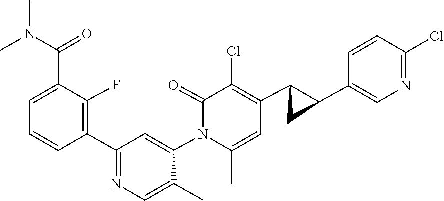 | Single known stereoisomer |
| 4.51 | 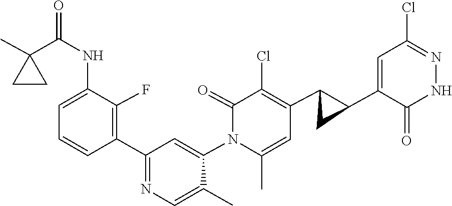 | Single known stereoisomer |
| 4.52 | 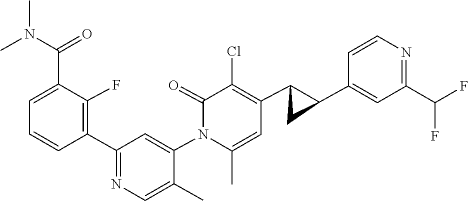 [SMILES: Cc1cnc(-c2cccc(C(=O)N(C)C)c2F)cc1-n1c(C)cc([C@H]2C[C@@H]2c2ccnc(C(F)F)c2)c(Cl)c1=O] | Mixture of atropisomers, single known cyclopropyl stereoisomer |
| 4.53 | 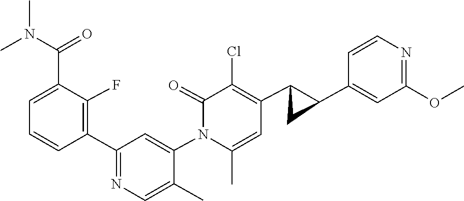 | Mixture of atropisomers, single known cyclopropyl stereoisomer |
| 4.54 | 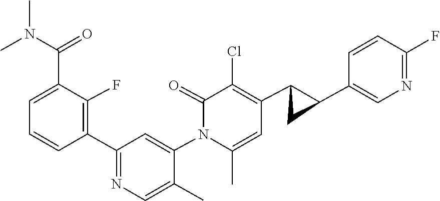 [SMILES: Cc1cnc(-c2cccc(C(=O)N(C)C)c2F)cc1-n1c(C)cc([C@H]2C[C@@H]2c2ccc(F)nc2)c(Cl)c1=O] | Mixture of atropisomers, single known cyclopropyl stereoisomer |
| 4.55 | 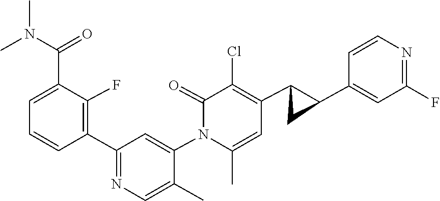 [SMILES: Cc1cnc(-c2cccc(C(=O)N(C)C)c2F)cc1-n1c(C)cc([C@H]2C[C@@H]2c2ccnc(F)c2)c(Cl)c1=O] | Mixture of atropisomers, single known cyclopropyl stereoisomer |
| 4.56 | 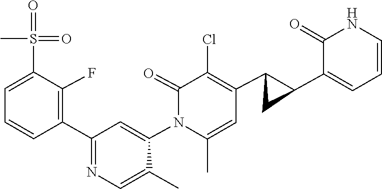 | Single known stereoisomer |
| 4.57 | 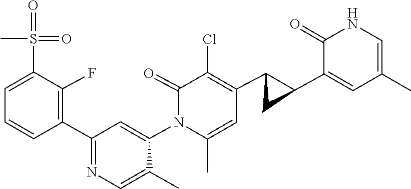 | Single known stereoisomer |
| 4.58 | 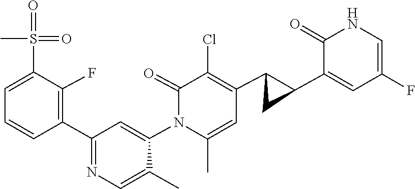 | Single known stereoisomer |
| 4.59 | 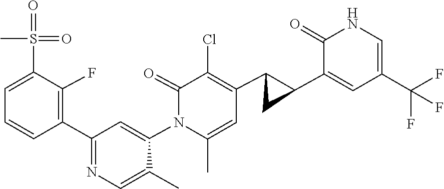 | Single known stereoisomer |
| 4.60 | 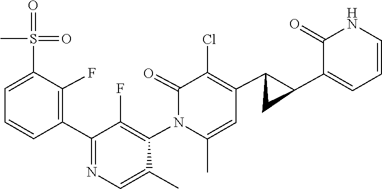 | Single known stereoisomer |
| 4.61 | 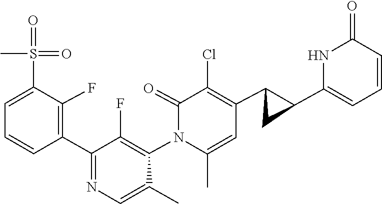 | Single known stereoisomer |
| 4.62 | 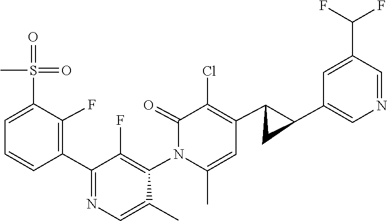 | Single known stereoisomer |
| 4.63 | 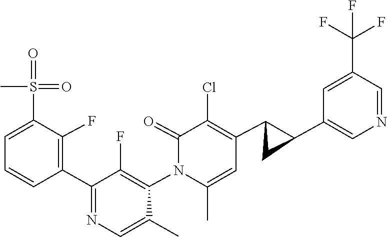 | Single known stereoisomer |
| 4.64 | 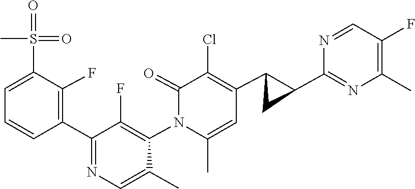 | Single known stereoisomer |
| 4.65 | 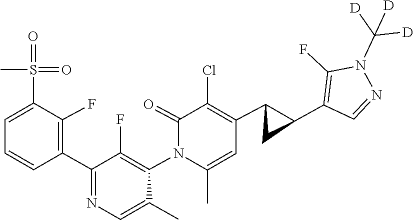 | Single known stereoisomer |
| 4.66 | 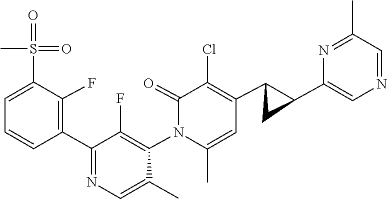 | Single known stereoisomer |
| 4.67 | 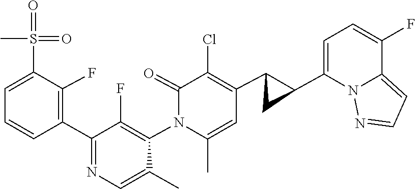 | Single known stereoisomer |
| 4.68 | 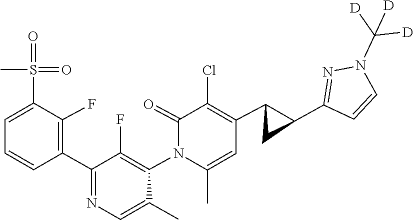 | Single known stereoisomer |
| 4.69 | 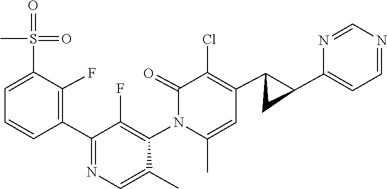 | Single known stereoisomer |
| 4.70 | 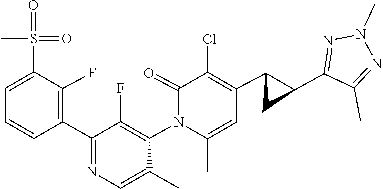 | Single known stereoisomer |
| 4.71 | 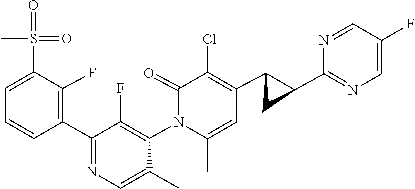 | Single known stereoisomer |
| 4.72 | 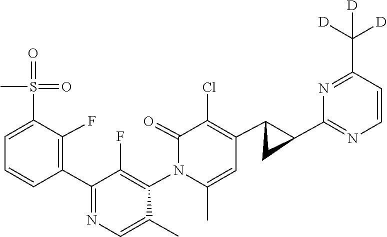 | Single known stereoisomer |
| 4.73 | 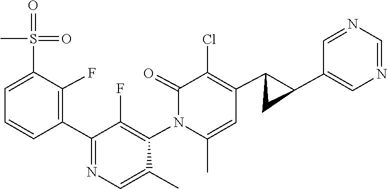 | Single known stereoisomer |
| 4.74 | 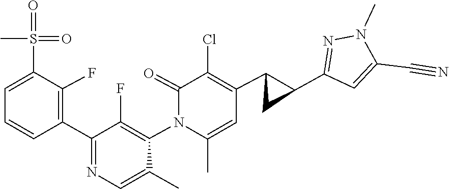 | Single known stereoisomer |
| 4.75 | 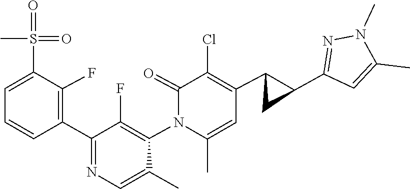 | Single known stereoisomer |
| 4.76 | 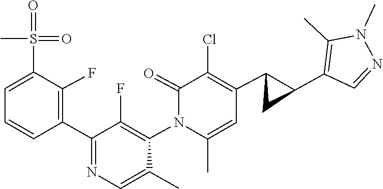 | Single known stereoisomer |
| 4.77 | 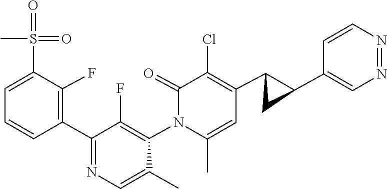 | Single known stereoisomer |
| 4.78 | 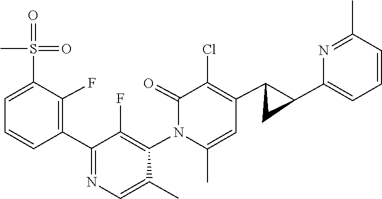 | Single known stereoisomer |
| 4.79 | 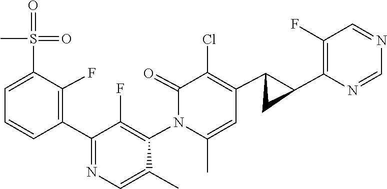 | Single known stereoisomer |
| 4.80 | 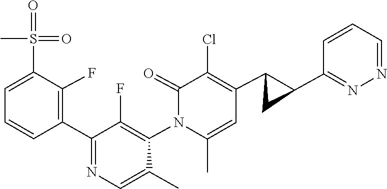 | Single known stereoisomer |
| 4.81 | 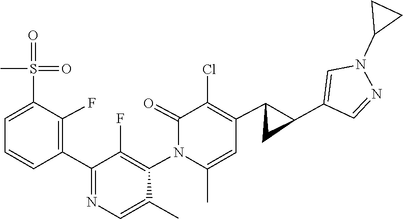 | Single known stereoisomer |
| 4.82 | 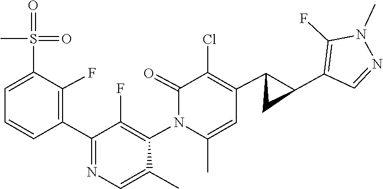 | Single known stereoisomer |
| 4.83 | 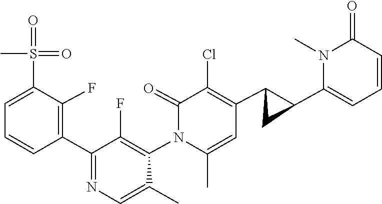 | Single known stereoisomer |
| 4.84 | 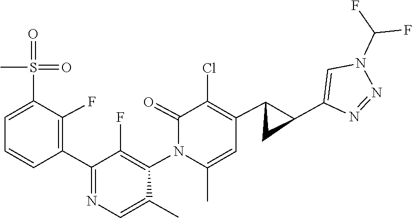 | Single known stereoisomer |
| 4.85 | 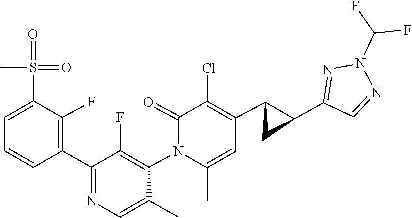 | Single known stereoisomer |
| 4.86 | 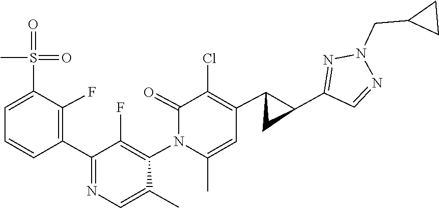 | Single known stereoisomer |
| 4.87 | 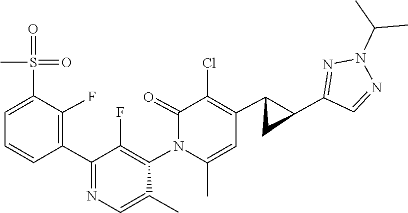 | Single known stereoisomer |
| 4.88 | 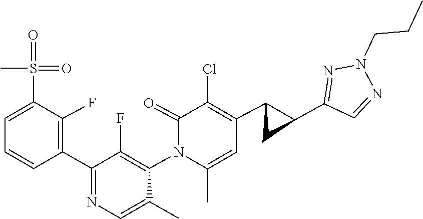 | Single known stereoisomer |
| 4.89 | 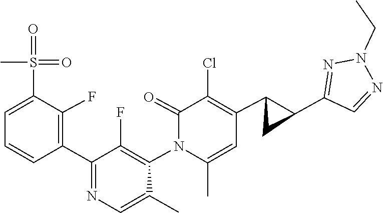 | Single known stereoisomer |
| 4.90 | 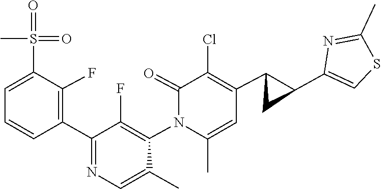 | Single known stereoisomer |
| 4.91 | 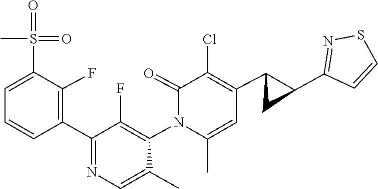 | Single known stereoisomer |
| 4.92 | 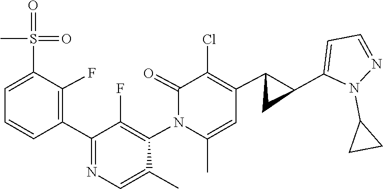 | Single known stereoisomer |
| 4.93 | 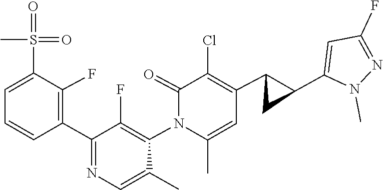 | Single known stereoisomer |
| 4.94 | 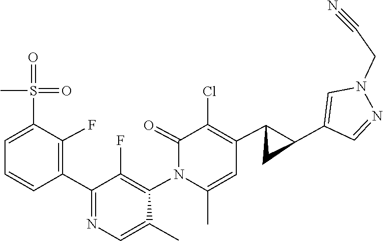 | Single known stereoisomer |
| 4.95 | 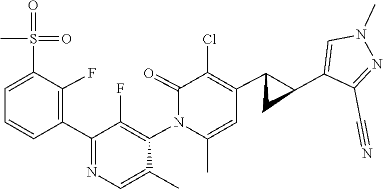 | Single known stereoisomer |
| 4.96 | 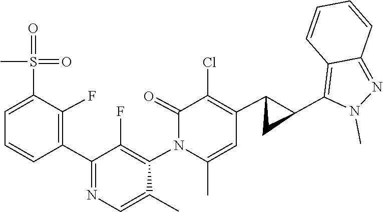 | Single known stereoisomer |
| 4.97 | 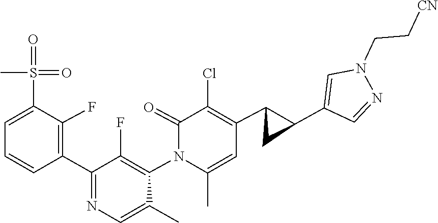 | Single known stereoisomer |
| 4.98 | 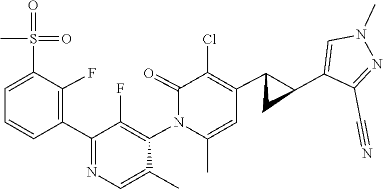 | Single known stereoisomer |
| 4.99 | 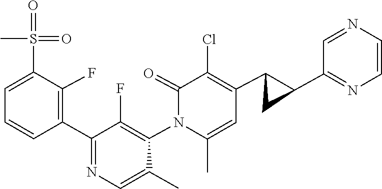 | Single known stereoisomer |
| 4.100 | 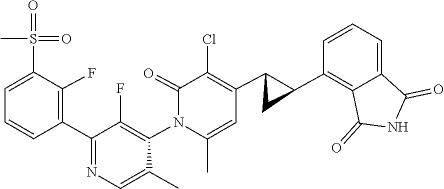 | Single known stereoisomer |
| 4.101 | 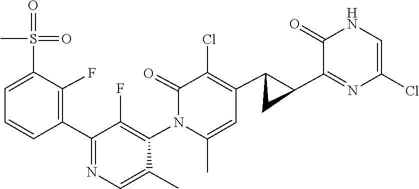 | Single known stereoisomer |
| 4.102 | 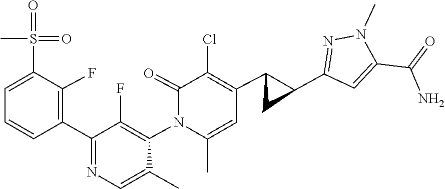 | Single known stereoisomer |
| 4.103 | 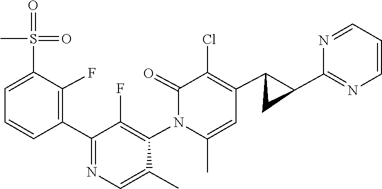 | Single known stereoisomer |
| 4.104 | 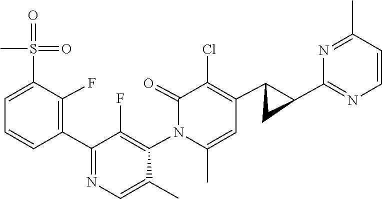 | Single known stereoisomer |
| 4.105 | 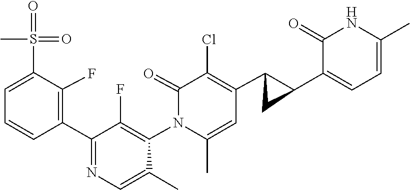 | Single known stereoisomer |
| 4.106 | 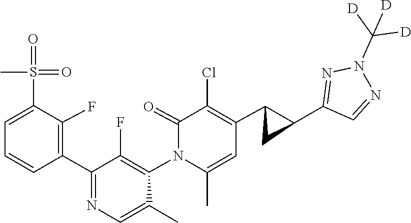 | Single known stereoisomer |
| 4.107 | 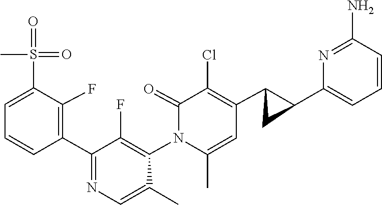 | Single known stereoisomer |
| 4.108 | 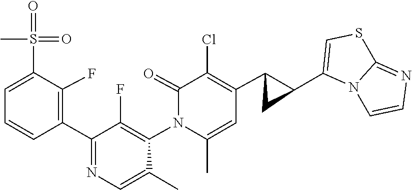 | Single known stereoisomer |
| 4.109 | 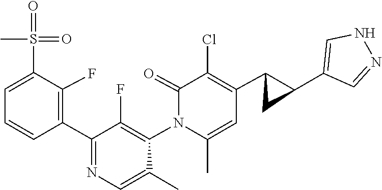 | Single known stereoisomer |
| 4.110 | 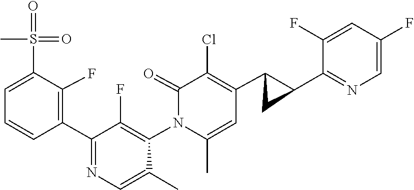 | Single known stereoisomer |
| 4.111 | 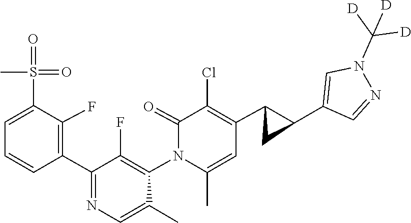 | Single known stereoisomer |
| 4.112 | 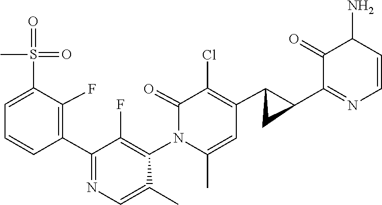 | Single known stereoisomer |
| 4.113 | 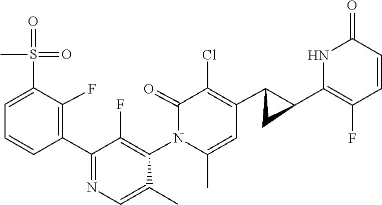 | Single known stereoisomer |
| 4.114 | 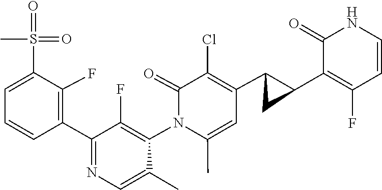 | Single known stereoisomer |
| 4.115 | 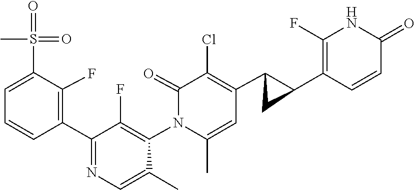 | Single known stereoisomer |
| 4.116 | 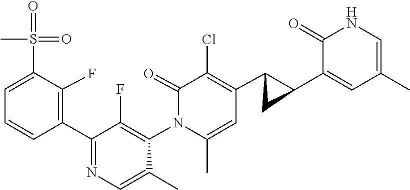 | Single known stereoisomer |
| 4.117 | 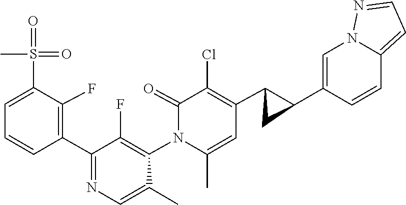 | Single known stereoisomer |
| 4.118 | 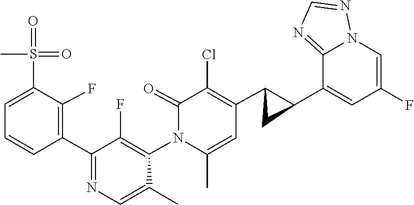 | Single known stereoisomer |
| 4.119 | 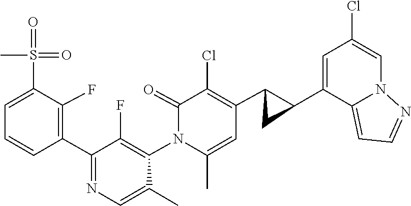 | Single known stereoisomer |
| 4.120 | 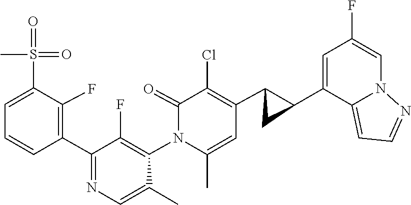 | Single known stereoisomer |
| 4.121 | 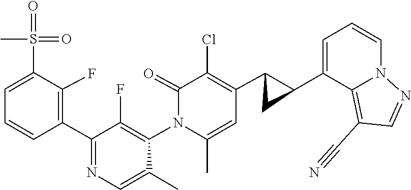 | Single known stereoisomer |
| 4.122 | 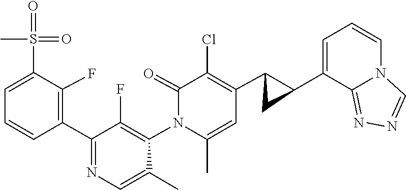 | Single known stereoisomer |
| 4.123 | 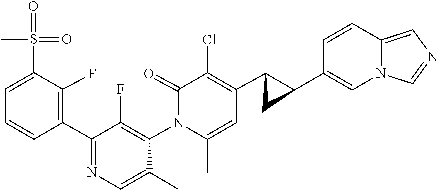 | Single known stereoisomer |
| 4.124 | 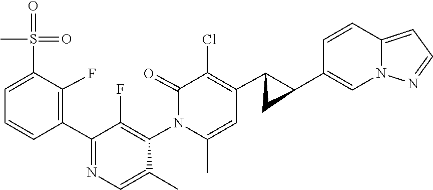 | Single known stereoisomer |
| 4.125 | 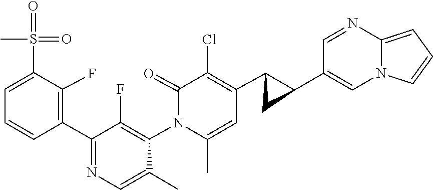 | Single known stereoisomer |
| 4.126 | 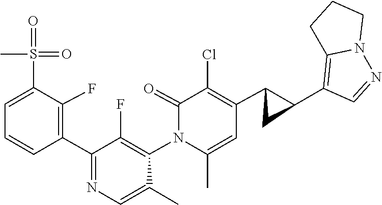 | Single known stereoisomer |
| 4.127 | 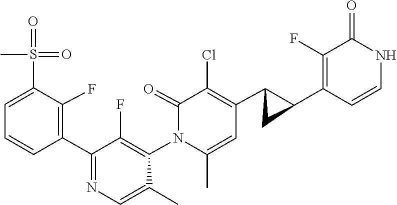 | Single known stereoisomer |
| 4.128 | 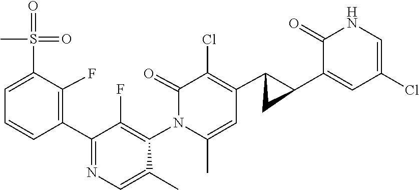 | Single known stereoisomer |
| 4.129 | 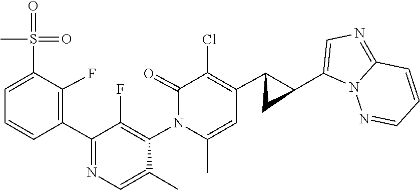 | Single known stereoisomer |
| 4.130 | 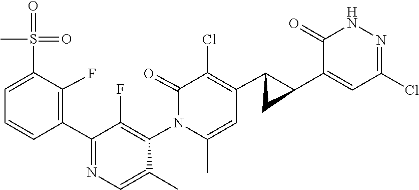 | Single known stereoisomer |
| 4.131 | 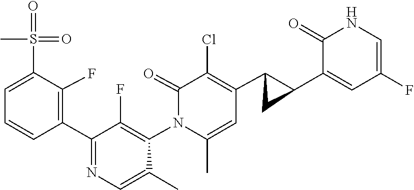 | Single known stereoisomer |
| 4.132 | 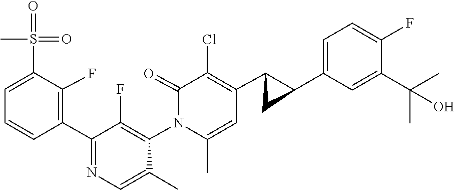 | Single known stereoisomer |
| 4.133 | 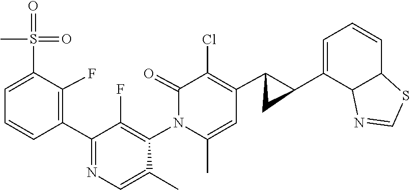 | Single known stereoisomer |
| 4.134 | 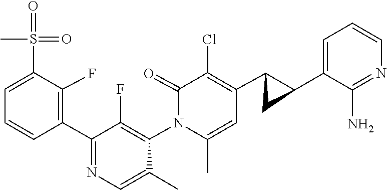 | Single known stereoisomer |
| 4.135 | 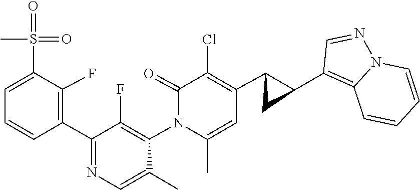 | Single known stereoisomer |
| 4.136 | 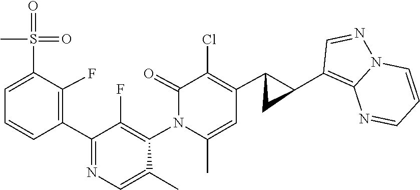 | Single known stereoisomer |
| 4.137 | 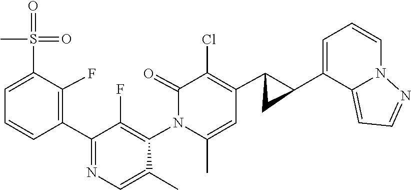 | Single known stereoisomer |
| 4.138 | 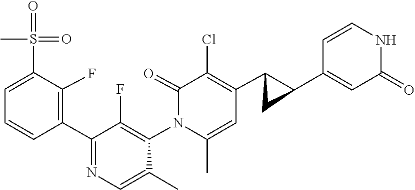 | Single known stereoisomer |
| 4.139 | 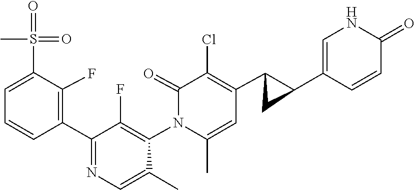 | Single known stereoisomer |
| 4.140 | 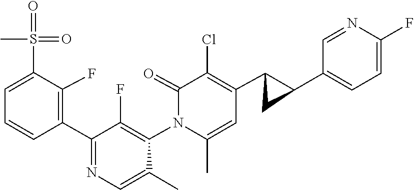 | Single known stereoisomer |
| 4.141 | 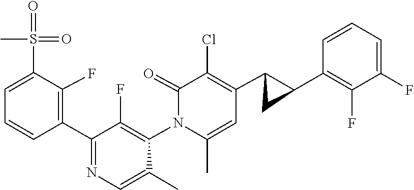 | Single known stereoisomer |
| 4.142 | 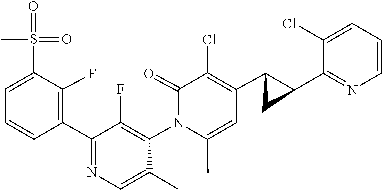 | Single known stereoisomer |
| 4.143 | 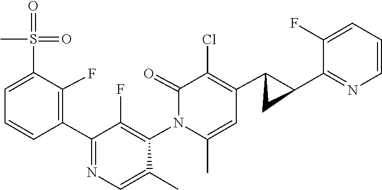 | Single known stereoisomer |
| 4.144 | 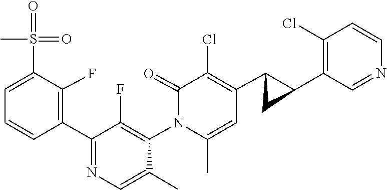 | Single known stereoisomer |
| 4.145 | 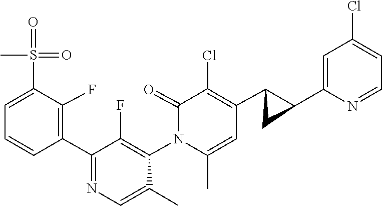 | Single known stereoisomer |
| 4.146 | 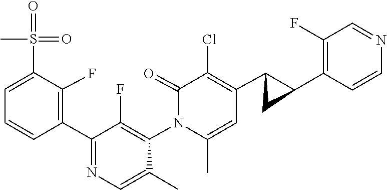 | Single known stereoisomer |
| 4.147 | 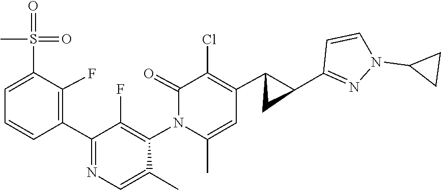 | Single known stereoisomer |
| 4.148 | 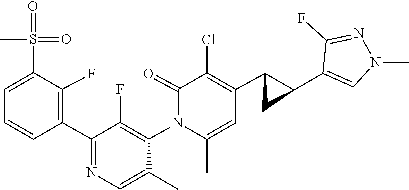 | Single known stereoisomer |
| 4.149 | 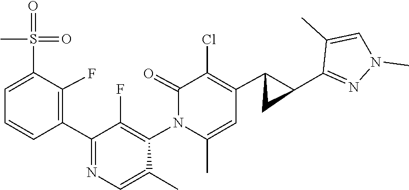 | Single known stereoisomer |
| 4.150 | 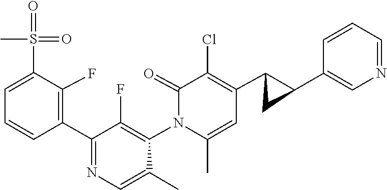 | Single known stereoisomer |
| 4.151 | 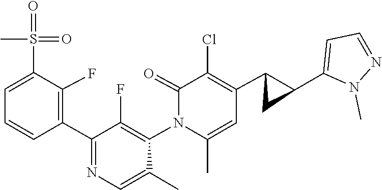 | Single known stereoisomer |
| 4.152 | 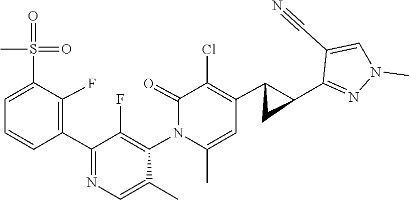 | Single known stereoisomer |
| 4.153 | 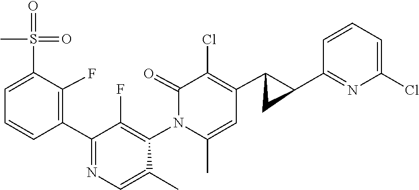 | Single known stereoisomer |
| 4.154 | 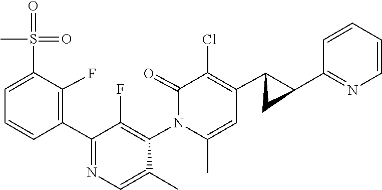 | Single known stereoisomer |
| 4.155 | 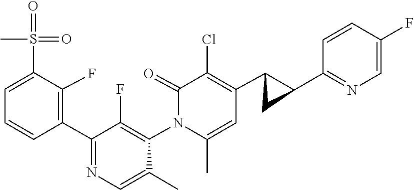 | Single known stereoisomer |
| 4.156 | 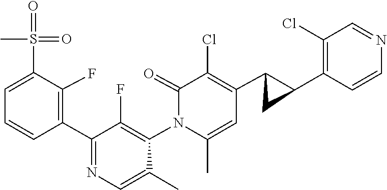 | Single known stereoisomer |
| 4.157 | 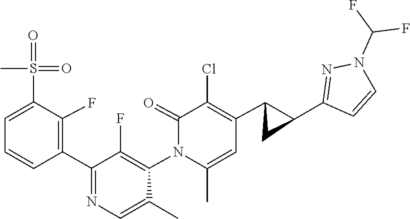 | Single known stereoisomer |
| 4.158 | 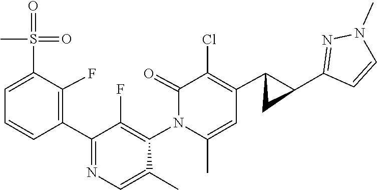 | Single known stereoisomer |
| 4.159 | 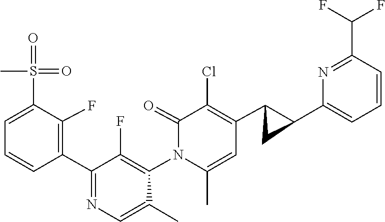 | Single known stereoisomer |
| 4.160 | 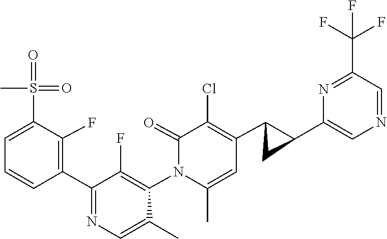 | Single known stereoisomer |
| 4.161 | 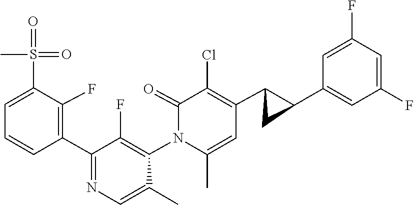 | Single known stereoisomer |
| 4.162 | 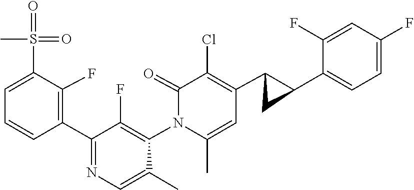 | Single known stereoisomer |
| 4.163 | 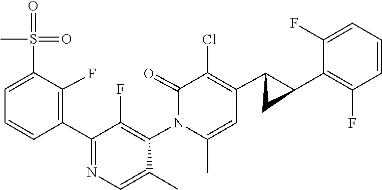 | Single known stereoisomer |
| 4.164 | 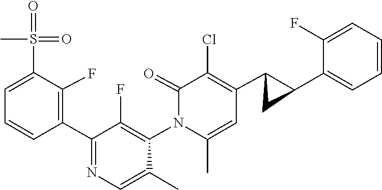 | Single known stereoisomer |
| 4.165 | 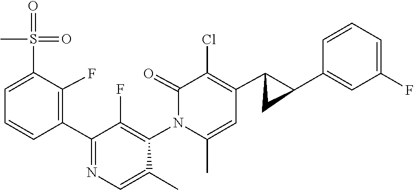 | Single known stereoisomer |
| 4.166 | 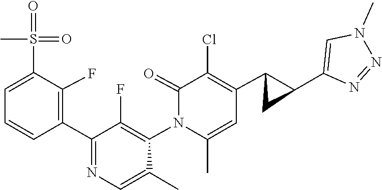 | Single known stereoisomer |
| 4.167 | 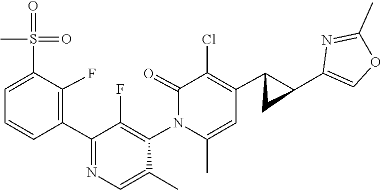 | Single known stereoisomer |
| 4.168 | 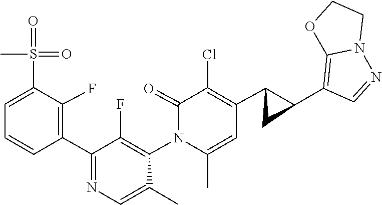 | Single known stereoisomer |
| 4.169 | 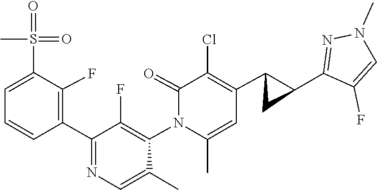 | Single known stereoisomer |
| 4.170 | 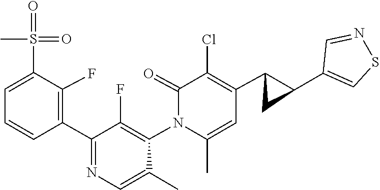 | Single known stereoisomer |
| 4.171 | 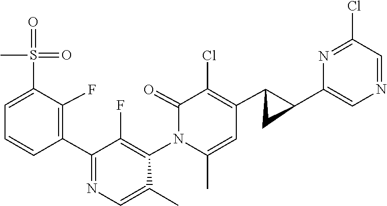 | Single known stereoisomer |
| 4.172 | 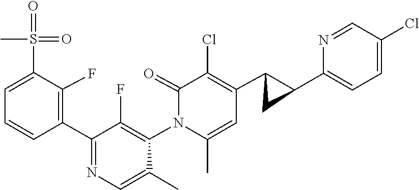 | Single known stereoisomer |
| 4.173 | 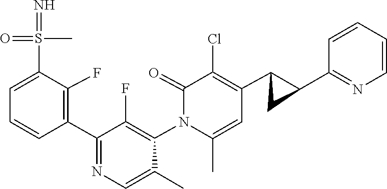 | Single unknown stereoisomer; single known atropisomer, single known cyclopropyl stereoisomer |
| 4.174 | 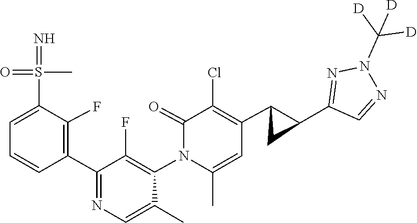 | Single unknown stereoisomer; single known atropisomer, single known cyclopropyl stereoisomer |
| 4.175 | 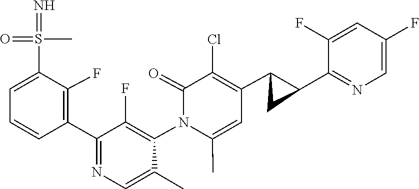 | Single unknown stereoisomer; single known atropisomer, single known cyclopropyl stereoisomer |
| 4.176 | 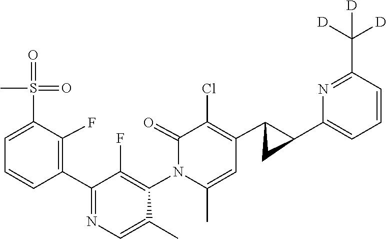 | Single known stereoisomer |
| 4.177 | 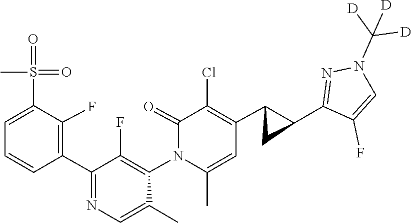 | Single known stereoisomer |
| 4.178 | 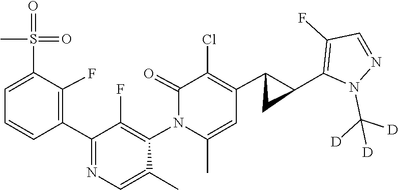 | Single known stereoisomer |
| 4.179 | 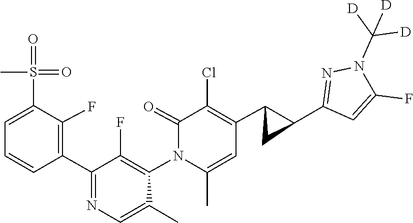 | Single known stereoisomer |
| 4.180 | 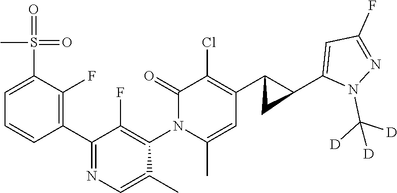 | Single known stereoisomer |
| 4.181 | 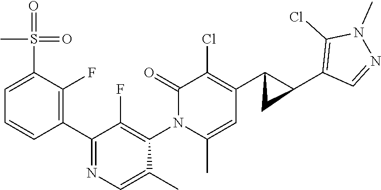 | Single known stereoisomer |
| 4.182 | 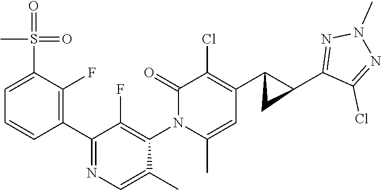 | Single known stereoisomer |
| 4.184 | 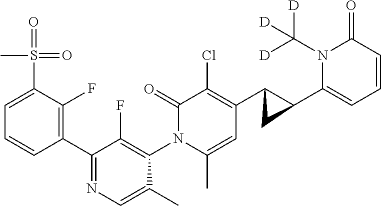 | Single known stereoisomer |
| 4.185 | 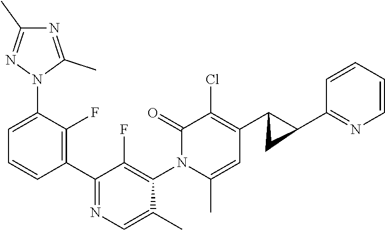 | Single known stereoisomer |
| 4.186 | 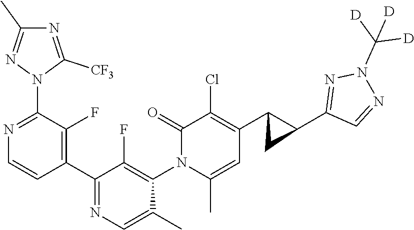 | Single known stereoisomer |
| 4.187 | 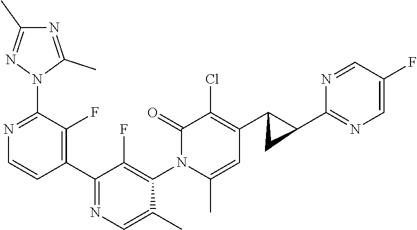 | Single known stereoisomer |
| 4.188 | 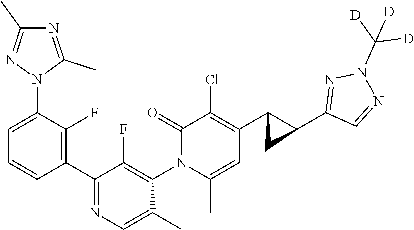 | Single known stereoisomer |
| 4.189 | 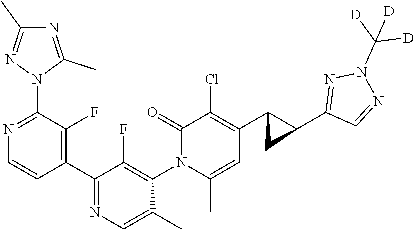 | Single known stereoisomer |
| 4.190 | 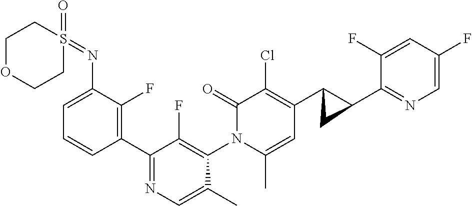 | Single known stereoisomer |
| 4.191 | 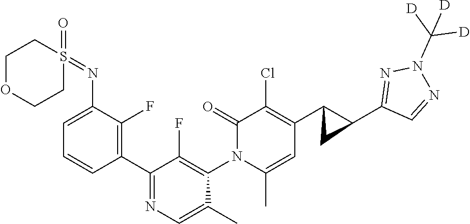 | Single known stereoisomer |
| 4.192 | 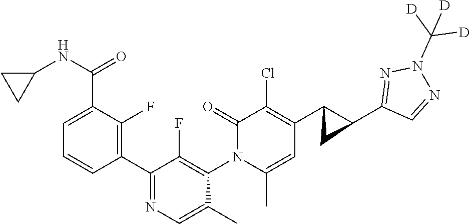 | Single known stereoisomer |
| 4.193 | 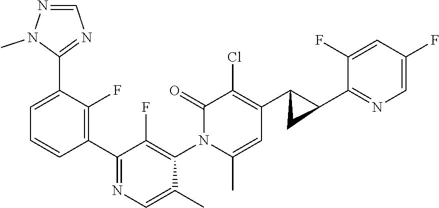 | Single known stereoisomer |
| 4.194 | 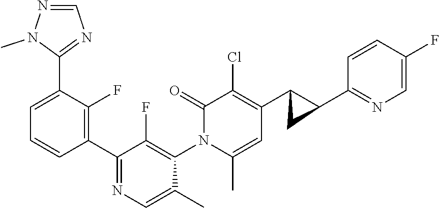 | Single known stereoisomer |
| 4.195 | 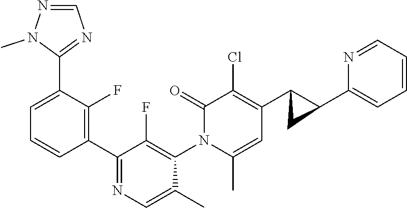 | Single known stereoisomer |
| 4.196 | 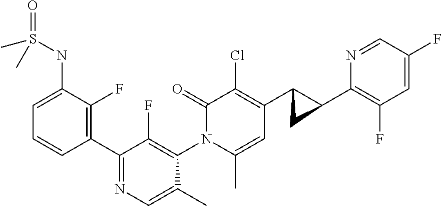 | Single known stereoisomer |
| 4.197 | 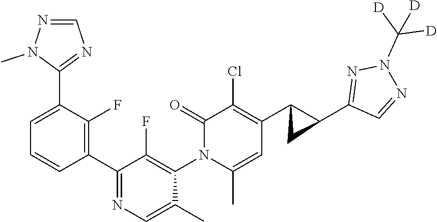 | Single known stereoisomer |
| 4.198 | 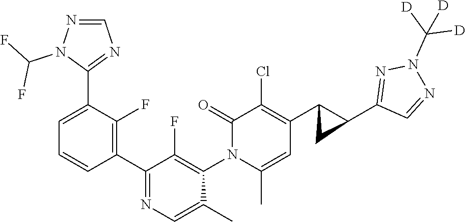 | Single known stereoisomer |
| 4.199 | 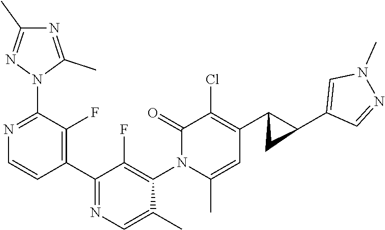 | Single known stereoisomer |
| 4.200 | 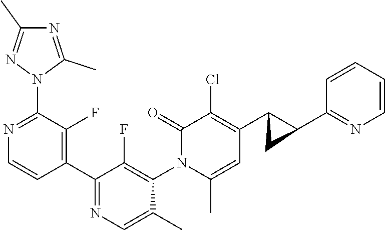 | Single known stereoisomer |
| 5.1 | 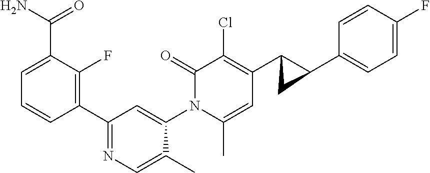 | Single known stereoisomer |
| 5.2 | 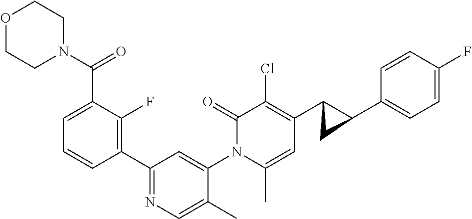 [SMILES: Cc1cnc(-c2cccc(C(=O)N3CCOCC3)c2F)cc1-n1c(C)cc([C@H]2C[C@@H]2c2ccc(F)cc2)c(Cl)c1=O] | Mixture of atropisomers, single known cyclopropyl stereoisomer |
| 5.3 | 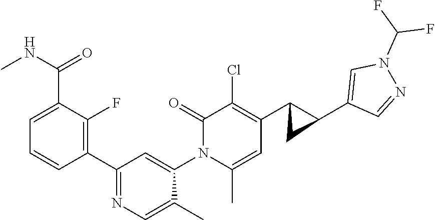 | Single known stereoisomer |
| 5.4 | 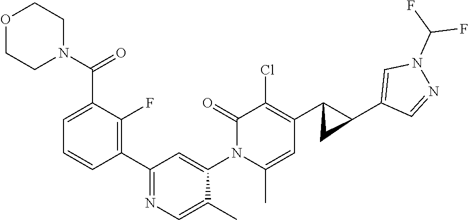 | Single known stereoisomer |
| 5.5 | 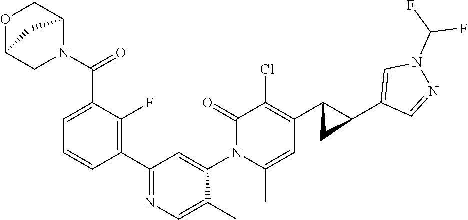 | Single known stereoisomer |
| 5.6 | 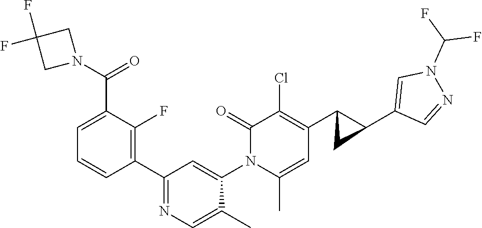 | Single known stereoisomer |
| 5.7 | 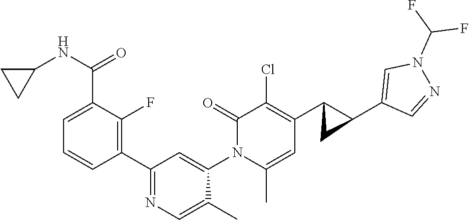 | Single known stereoisomer |
| 5.8 | 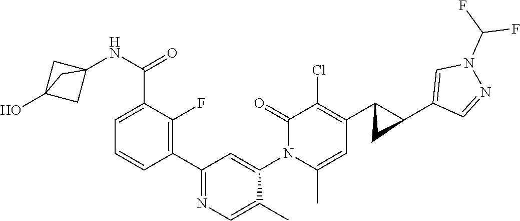 | Single known stereoisomer |
| 5.9 | 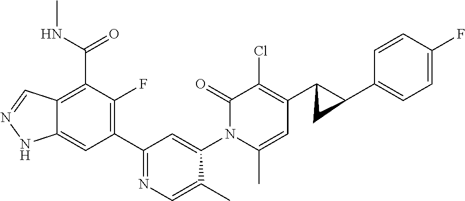 | Single known stereoisomer |
| 5.10 | 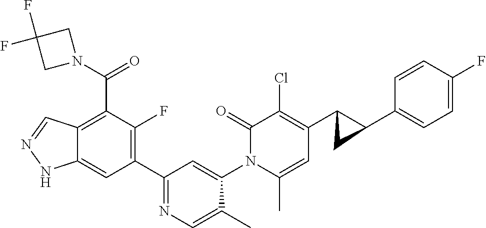 | Single known stereoisomer |
| 5.11 | 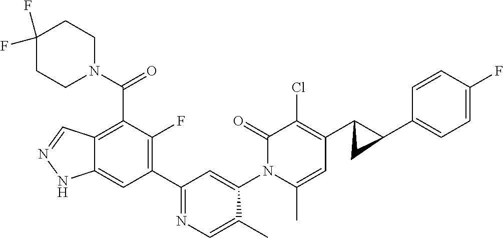 | Single known stereoisomer |
| 5.12 | 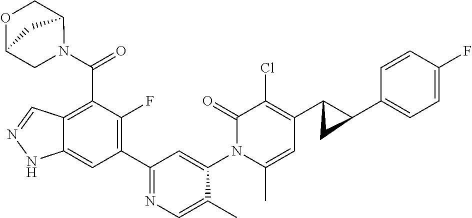 | Single known stereoisomer |
| 5.13 | 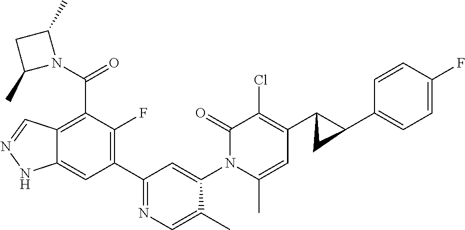 | Single known stereoisomer |
| 5.14 | 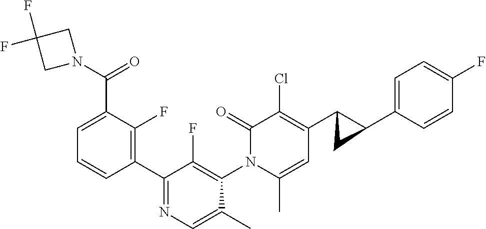 | Single known stereoisomer |
| 5.15 | 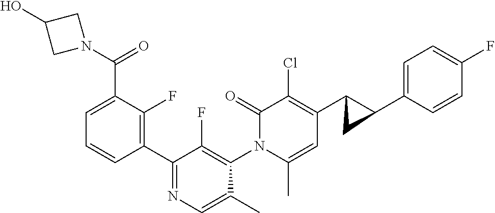 | Single known stereoisomer |
| 5.16 | 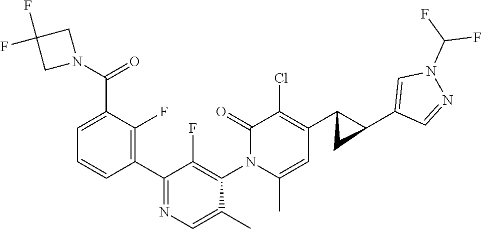 | Single known stereoisomer |
| 5.17 | 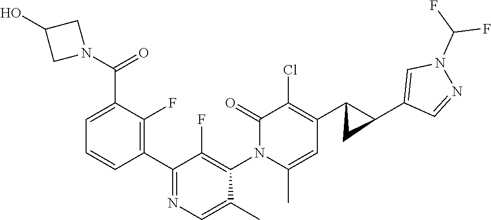 | Single known stereoisomer |
| 5.18 | 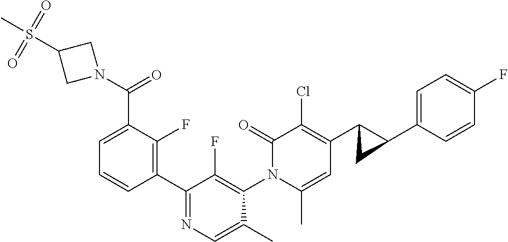 | Single known stereoisomer |
| 5.19 | 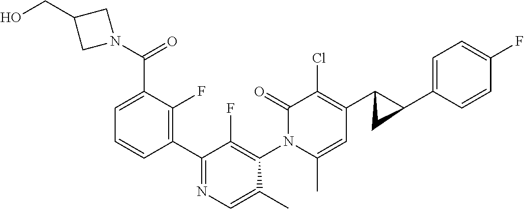 | Single known stereoisomer |
| 5.20 | 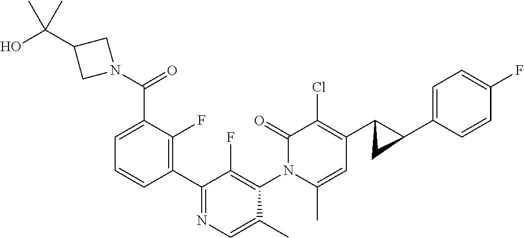 | Single known stereoisomer |
| 5.21 | 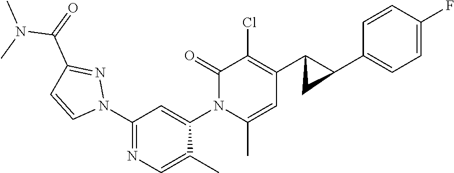 | Single known stereoisomer |
| 5.22 | 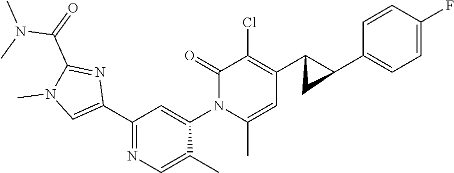 | Single known stereoisomer |
| 5.23 | 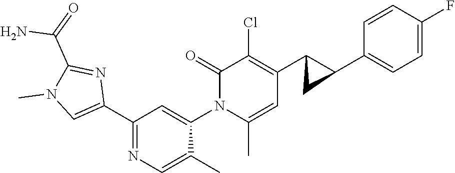 | Single known stereoisomer |
| 5.24 | 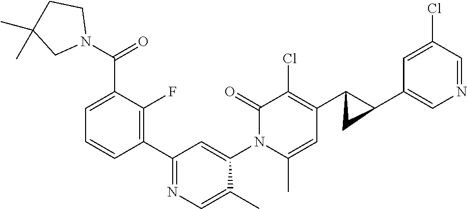 | Single known stereoisomer |
| 5.25 | 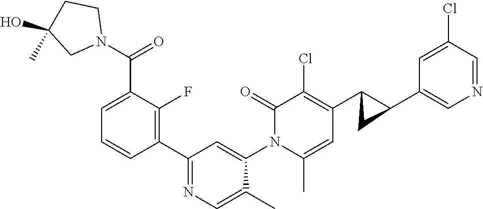 | Single known stereoisomer |
| 5.26 | 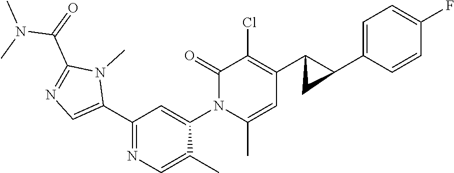 | Single known stereoisomer |
| 5.27 | 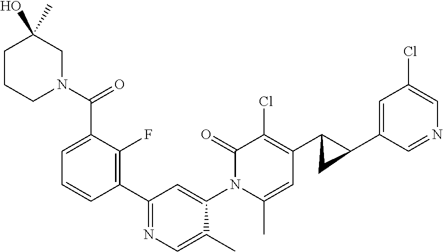 | Single known stereoisomer |
| 5.28 | 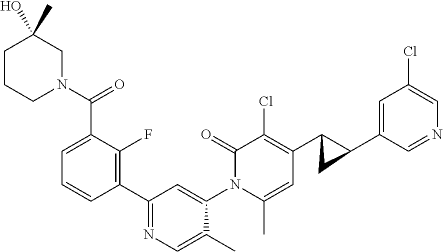 | Single known stereoisomer |
| 5.29 | 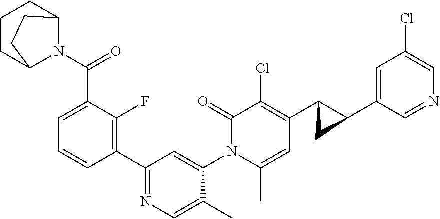 | Single known stereoisomer |
| 5.30 | 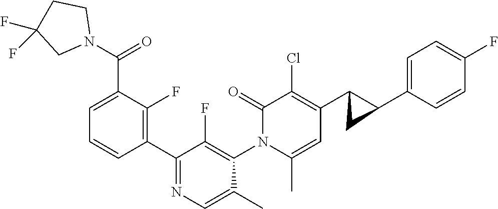 | Single known stereoisomer |
| 5.31 | 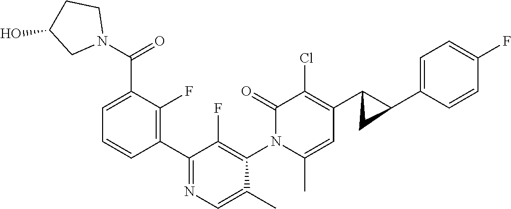 | Single known stereoisomer |
| 5.32 | 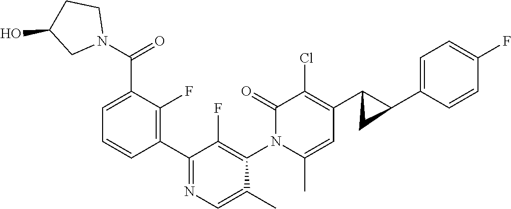 | Single known stereoisomer |
| 5.33 | 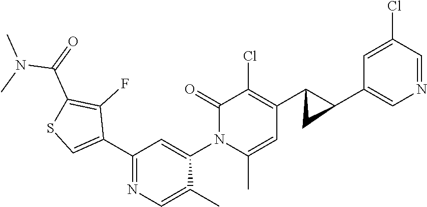 | Single known stereoisomer |
| 5.34 | 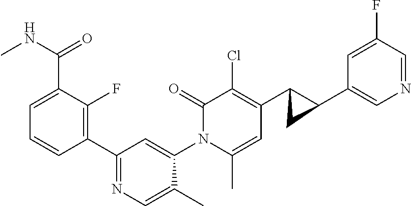 | Single known stereoisomer |
| 5.35 | 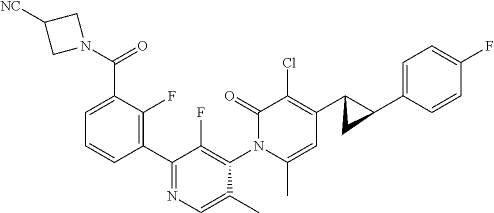 | Single known stereoisomer |
| 5.36 | 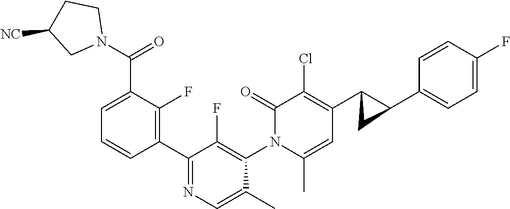 | Single known stereoisomer |
| 5.37 | 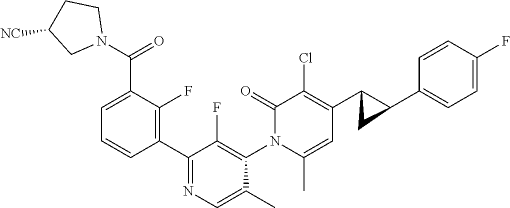 | Single known stereoisomer |
| 5.38 | 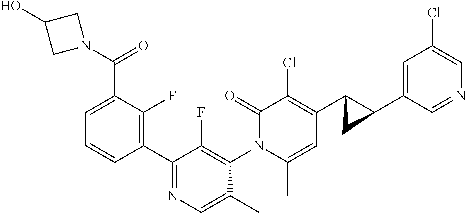 | Single known stereoisomer |
| 5.39 | 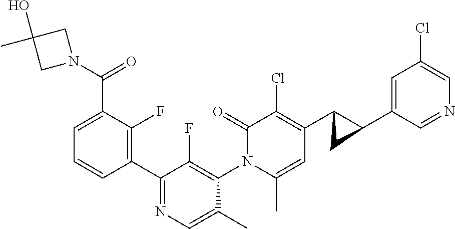 | Single known stereoisomer |
| 5.40 | 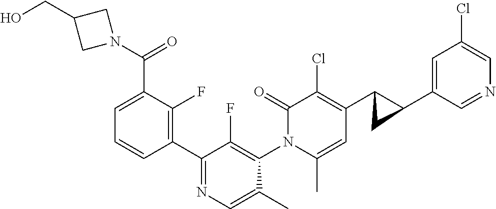 | Single known stereoisomer |
| 5.41 | 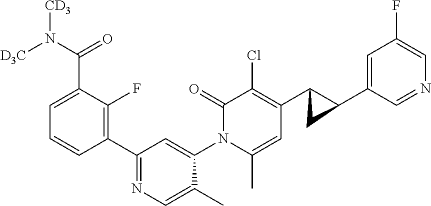 | Single known stereoisomer |
| 5.42 | 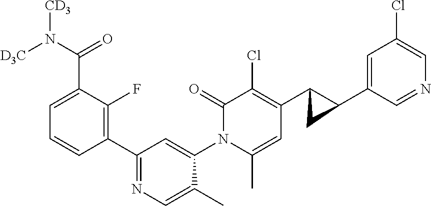 | Single known stereoisomer |
| 5.43 | 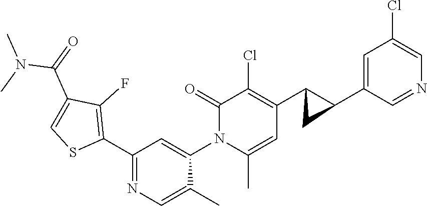 | Single known stereoisomer |
| 5.44 | 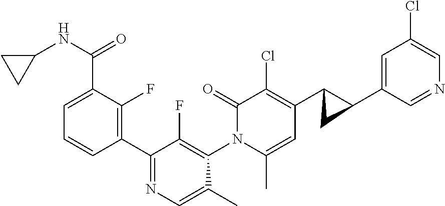 | Single known stereoisomer |
| 5.45 | 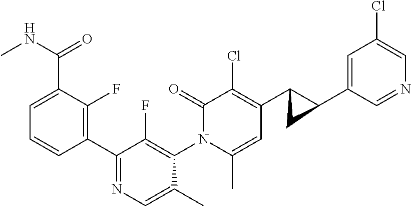 | Single known stereoisomer |
| 5.46 | 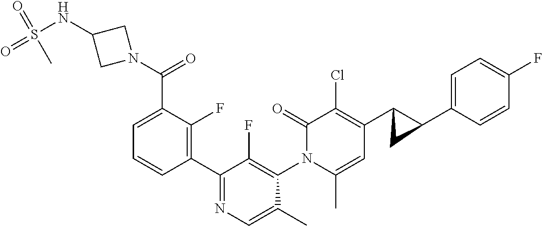 | Single known stereoisomer |
| 5.47 | 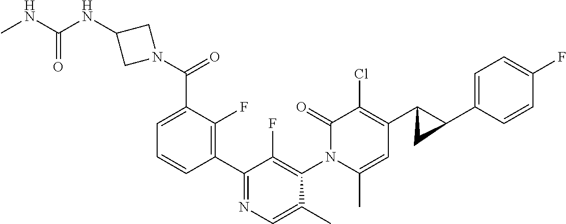 | Single known stereoisomer |
| 5.48 | 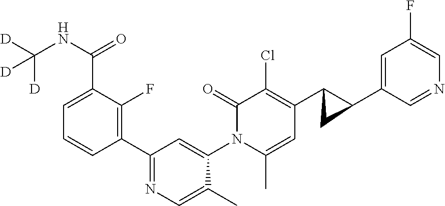 | Single known stereoisomer |
| 5.49 | 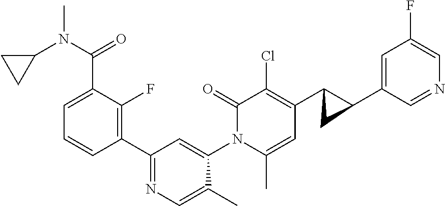 | Single known stereoisomer |
| 5.50 | 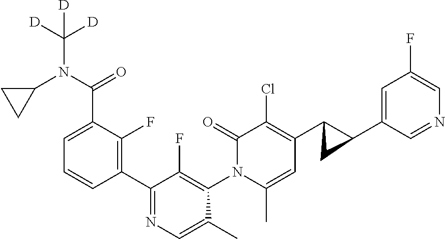 | Single known stereoisomer |
| 5.51 | 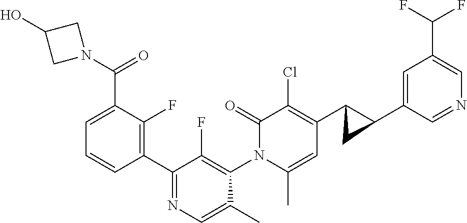 | Single known stereoisomer |
| 5.52 | 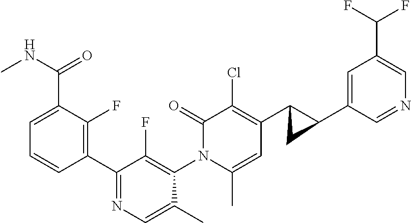 | Single known stereoisomer |
| 5.53 | 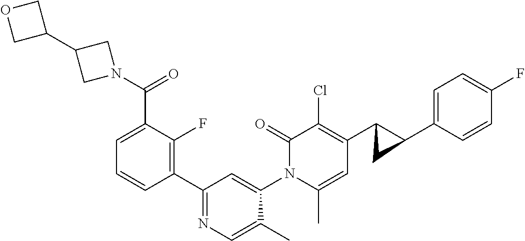 | Single known stereoisomer |
| 5.54 | 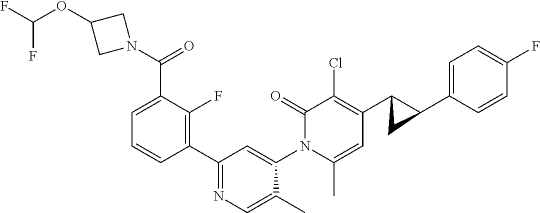 | Single known stereoisomer |
| 5.55 | 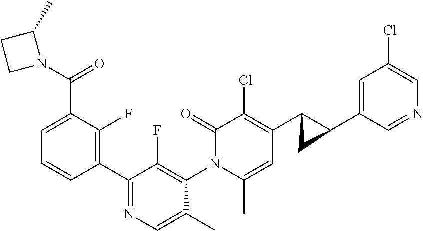 | Single known stereoisomer |
| 5.56 | 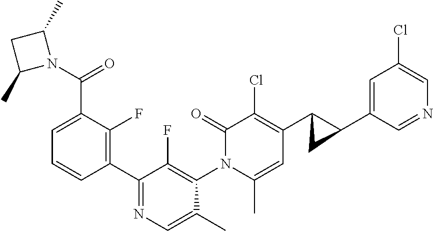 | Single known stereoisomer |
| 5.57 | 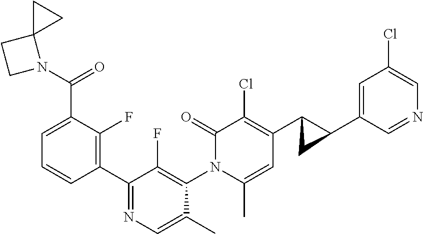 | Single known stereoisomer |
| 5.58 | 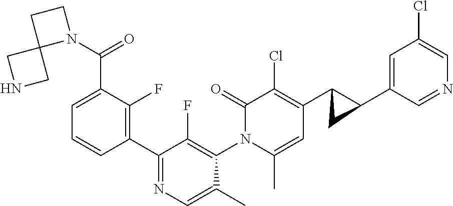 | Single known stereoisomer |
| 5.59 | 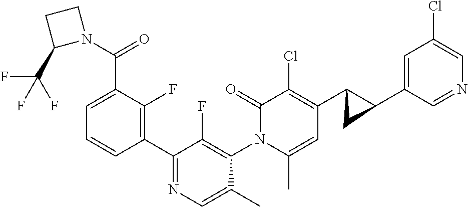 | Single known stereoisomer |
| 5.60 | 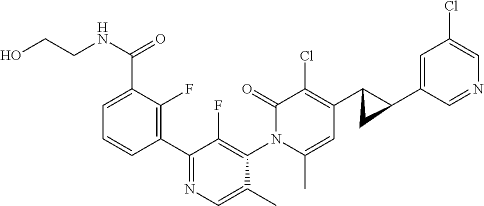 | Single known stereoisomer |
| 5.61 | 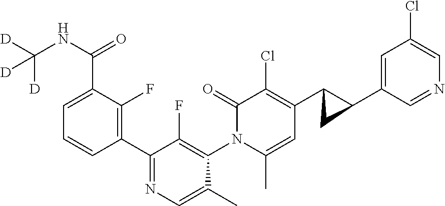 | Single known stereoisomer |
| 5.62 | 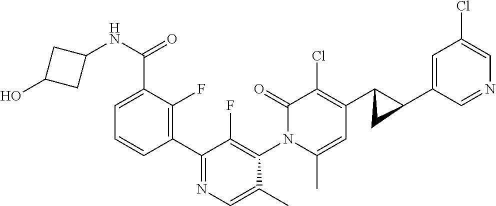 | Mixture of diastereomers, single known atropisomer, single known cyclopropyl stereoisomer |
| 5.63 | 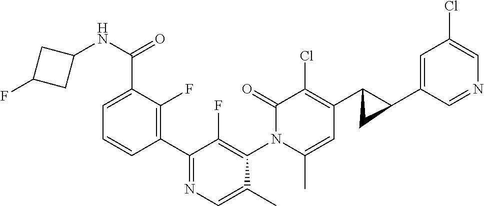 | Mixture of diastereomers, single known atropisomer, single known cyclopropyl stereoisomer |
| 6.1 | 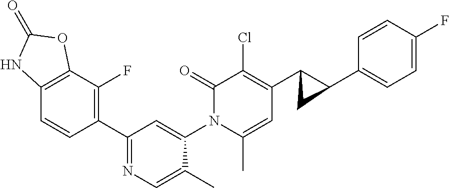 | Single known stereoisomer |
| 6.2 | 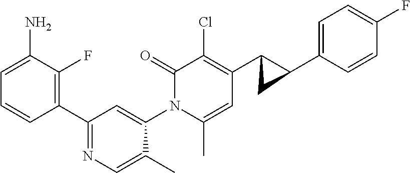 | Single known stereoisomer |
| 6.3 | 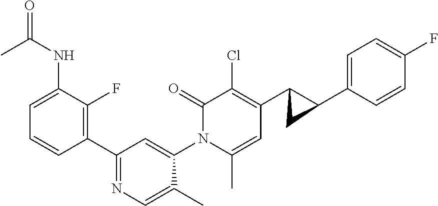 | Single known stereoisomer |
| 6.4 | 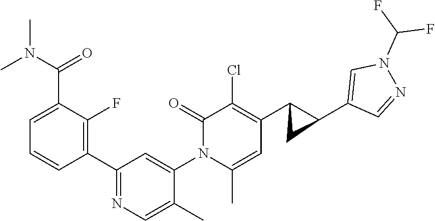 [SMILES: Cc1cnc(-c2cccc(C(=O)N(C)C)c2F)cc1-n1c(C)cc([C@H]2C[C@@H]2c2cnn(C(F)F)c2)c(Cl)c1=O] | Mixture of atropisomers, single known cyclopropyl stereoisomer |
| 6.5 | 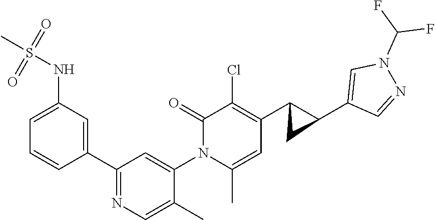 [SMILES: Cc1cnc(-c2cccc(NS(C)(=O)=O)c2)cc1-n1c(C)cc([C@H]2C[C@@H]2c2cnn(C(F)F)c2)c(Cl)c1=O] | Mixture of atropisomers, single known cyclopropyl stereoisomer |
| 6.6 | 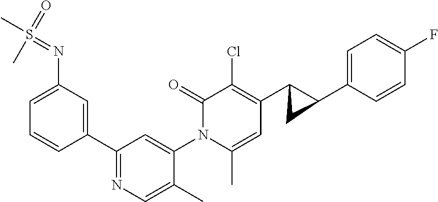 [SMILES: Cc1cnc(-c2cccc(N=S(C)(C)=O)c2)cc1-n1c(C)cc([C@H]2C[C@@H]2c2ccc(F)cc2)c(Cl)c1=O] | Mixture of atropisomers, single known cyclopropyl stereoisomer |
| 6.7 | 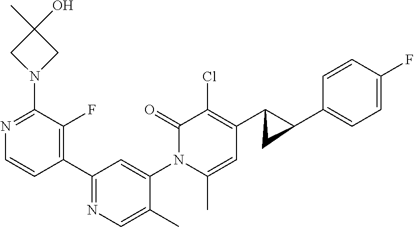 [SMILES: Cc1cnc(-c2ccnc(N3CC(C)(O)C3)c2F)cc1-n1c(C)cc([C@H]2C[C@@H]2c2ccc(F)cc2)c(Cl)c1=O] | Mixture of atropisomers, single known cyclopropyl stereoisomer |
| 6.8 | 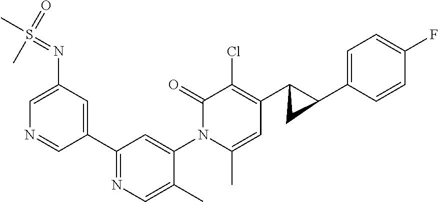 [SMILES: Cc1cnc(-c2cncc(N=S(C)(C)=O)c2)cc1-n1c(C)cc([C@H]2C[C@@H]2c2ccc(F)cc2)c(Cl)c1=O] | Mixture of atropisomers, single known cyclopropyl stereoisomer |
| 6.9 | 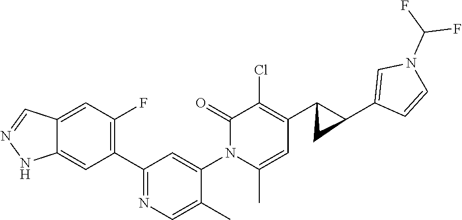 [SMILES: Cc1cnc(-c2cc3[nH]ncc3cc2F)cc1-n1c(C)cc([C@H]2C[C@@H]2c2ccn(C(F)F)c2)c(Cl)c1=O] | Mixture of atropisomers, single known cyclopropyl stereoisomer |
| 6.10 | 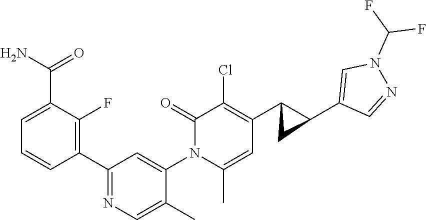 [SMILES: Cc1cnc(-c2cccc(C(N)=O)c2F)cc1-n1c(C)cc([C@H]2C[C@@H]2c2cnn(C(F)F)c2)c(Cl)c1=O] | Mixture of atropisomers, single known cyclopropyl stereoisomer |
| 6.11 | 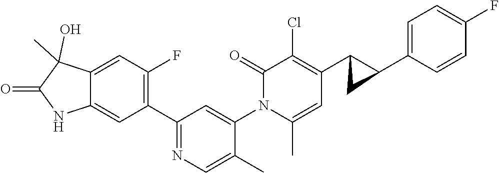 [SMILES: Cc1cnc(-c2cc3c(cc2F)C(C)(O)C(=O)N3)cc1-n1c(C)cc([C@H]2C[C@@H]2c2ccc(F)cc2)c(Cl)c1=O] | Mixture of atropisomers, single known cyclopropyl stereoisomer |
| 6.12 | 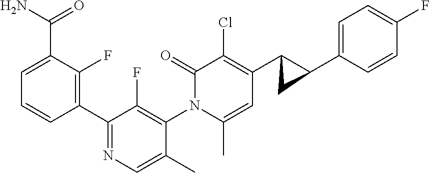 [SMILES: Cc1cnc(-c2cccc(C(N)=O)c2F)c(F)c1-n1c(C)cc([C@H]2C[C@@H]2c2ccc(F)cc2)c(Cl)c1=O] | Mixture of atropisomers, single known cyclopropyl stereoisomer |
| 6.13 | 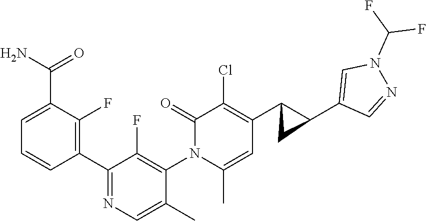 [SMILES: Cc1cnc(-c2cccc(C(N)=O)c2F)c(F)c1-n1c(C)cc([C@H]2C[C@@H]2c2cnn(C(F)F)c2)c(Cl)c1=O] | Mixture of atropisomers, single known cyclopropyl stereoisomer |
| 6.14 | 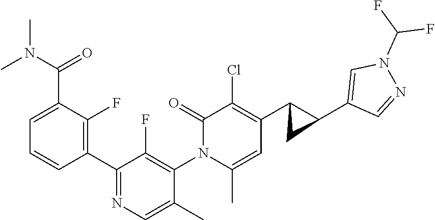 [SMILES: Cc1cnc(-c2cccc(C(=O)N(C)C)c2F)c(F)c1-n1c(C)cc([C@H]2C[C@@H]2c2cnn(C(F)F)c2)c(Cl)c1=O] | Mixture of atropisomers, single known cyclopropyl stereoisomer |
| 6.15 | 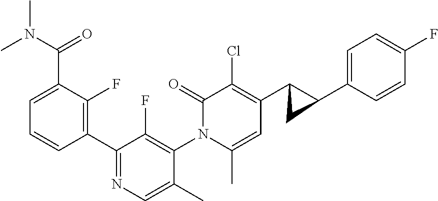 [SMILES: Cc1cnc(-c2cccc(C(=O)N(C)C)c2F)c(F)c1-n1c(C)cc([C@H]2C[C@@H]2c2ccc(F)cc2)c(Cl)c1=O] | Mixture of atropisomers, single known cyclopropyl stereoisomer |
| 6.16 | 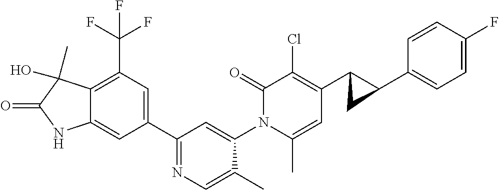 | Mixture of alcohol stereoisomers, single known atropisomer, single known cyclopropyl stereoisomer |
| 6.17 | 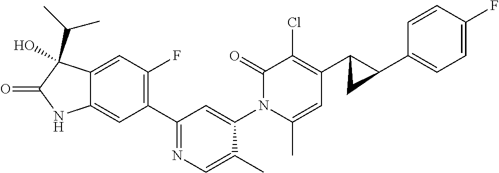 | Single unknown stereoisomer, single known atropisomer, single known cyclopropyl stereoisomer |
| 6.18 | 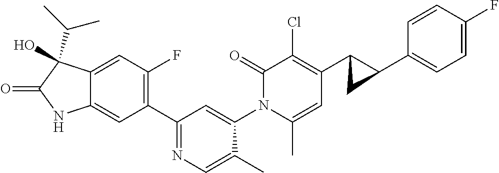 | Single unknown stereoisomer, single known atropisomer, single known cyclopropyl stereoisomer |
| 6.19 | 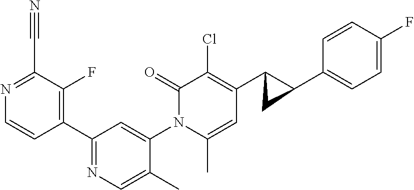 [SMILES: Cc1cnc(-c2ccnc(C#N)c2F)cc1-n1c(C)cc([C@H]2C[C@@H]2c2ccc(F)cc2)c(Cl)c1=O] | Mixture of atropisomers, single known cyclopropyl stereoisomer |
| 6.20 | 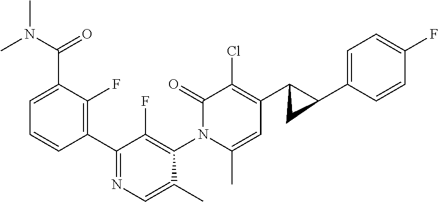 | Single known stereoisomer |
| 6.21 | 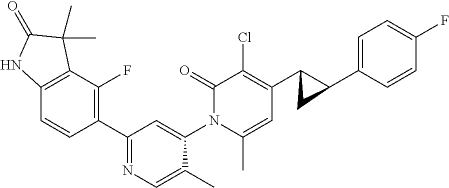 | Single known stereoisomer |
| 6.22 | 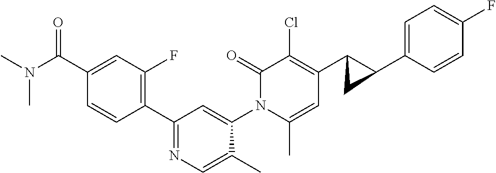 | Single known stereoisomer |
| 6.23 | 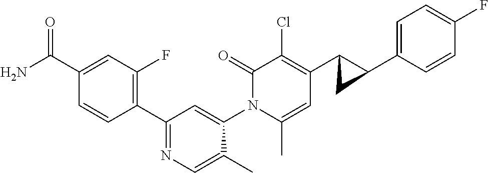 | Single known stereoisomer |
| 6.24 | 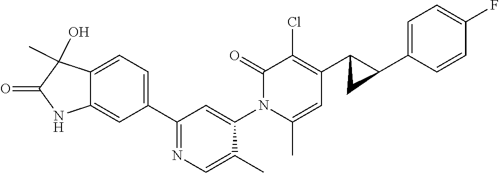 | Mixture of diastereomers, single known atropisomer, single known cyclopropyl stereoisomer |
| 6.25 | 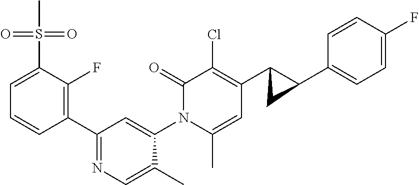 | Single known stereoisomer |
| 6.26 | 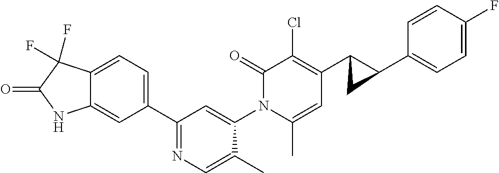 | Single known stereoisomer |
| 6.27 | 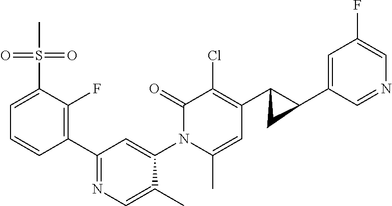 | Single known stereoisomer |
| 6.28 | 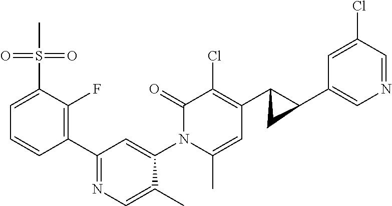 | Single known stereoisomer |
| 6.29 | 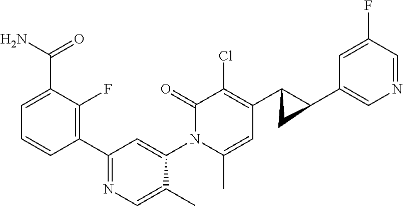 | Single known stereoisomer |
| 6.30 | 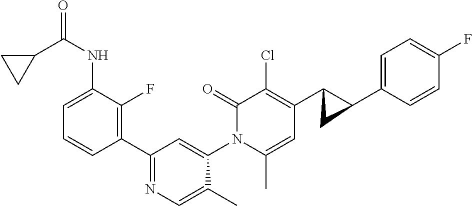 | Single known stereoisomer |
| 6.31 | 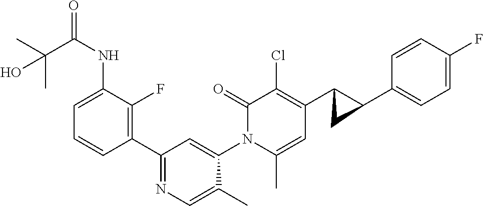 | Single known stereoisomer |
| 6.32 | 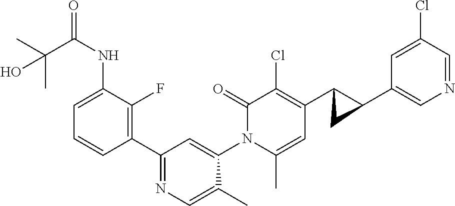 | Single known stereoisomer |
| 6.33 | 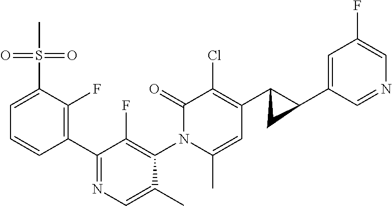 | Single known stereoisomer |
| 6.34 | 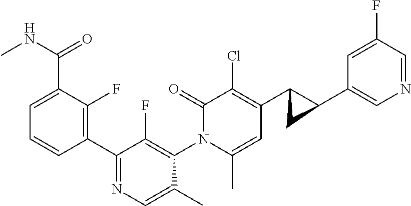 | Single known stereoisomer |
| 6.35 | 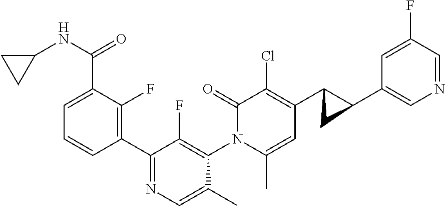 | Single known stereoisomer |
| 6.36 | 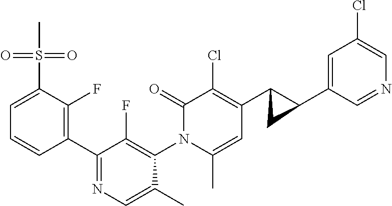 | Single known stereoisomer |
| 6.37 | 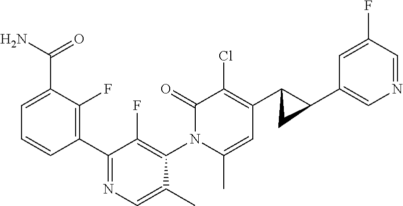 | Single known stereoisomer |
| 6.38 | 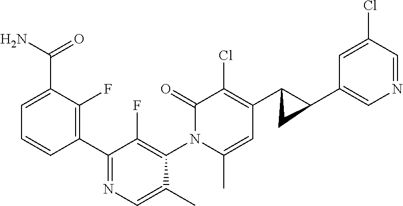 | Single known stereoisomer |
| 6.39 | 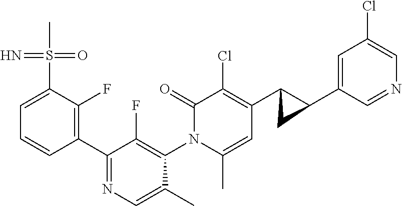 | Mixture of diastereomers, single known atropisomer, single known cyclopropyl stereoisomer |
| 6.40 | 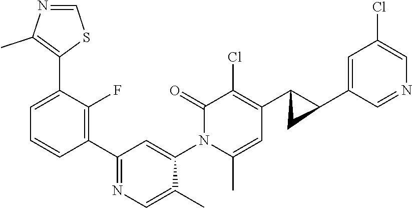 | Single known stereoisomer |
| 6.41 | 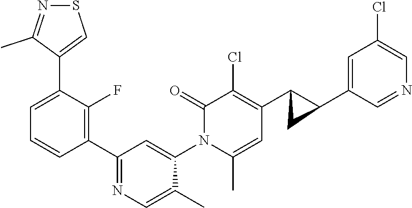 | Single known stereoisomer |
| 6.42 | 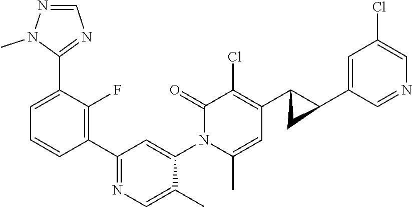 | Single known stereoisomer |
| 6.43 | 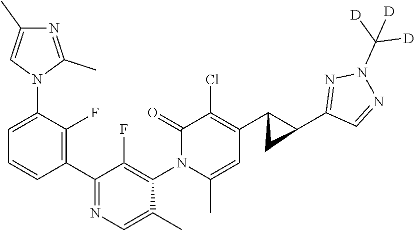 | Single known stereoisomer |
| 6.44 | 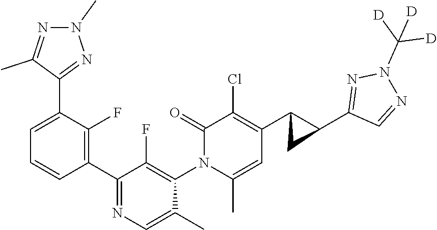 | Single known stereoisomer |
| 6.45 | 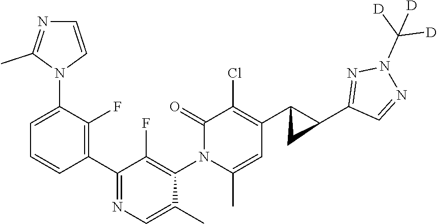 | Single known stereoisomer |
| 6.46 | 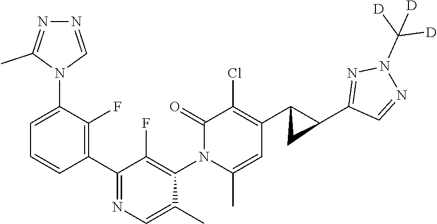 | Single known stereoisomer |
| 6.47 | 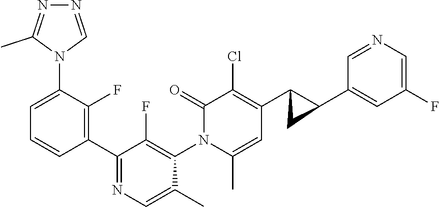 | Single known stereoisomer |
| 6.48 | 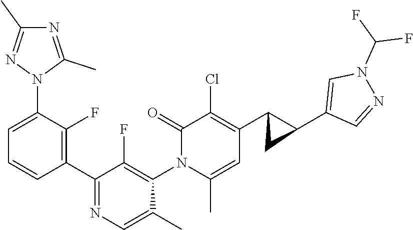 | Single known stereoisomer |
| 6.49 | 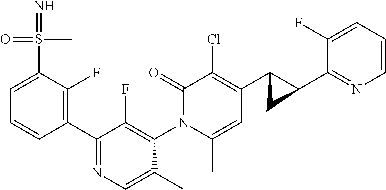 | Single unknown stereoisomer, single known atropisomer, single known cyclopropyl stereoisomer |
| 6.50 | 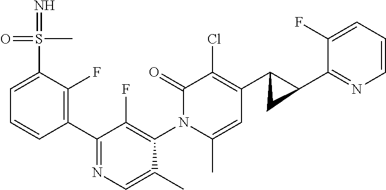 | Single unknown stereoisomer, single known atropisomer, single known cyclopropyl stereoisomer |
| 6.51 | 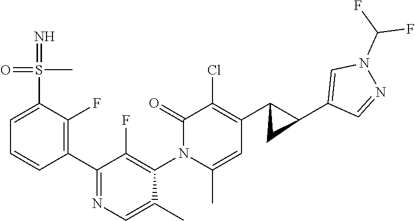 | Single unknown stereoisomer, single known atropisomer, single known cyclopropyl stereoisomer |
| 6.52 | 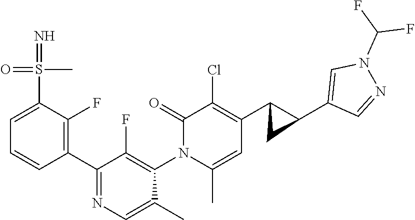 | Single unknown stereoisomer, single known atropisomer, single known cyclopropyl stereoisomer |
| 6.53 | 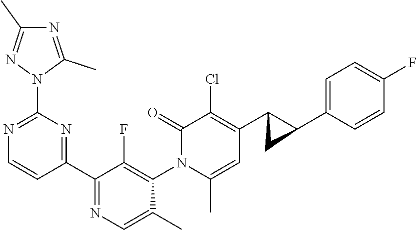 | Single known stereoisomer |
| 6.54 | 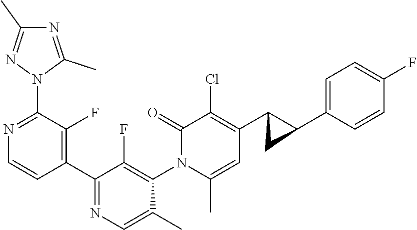 | Single known stereoisomer |
| 6.55 | 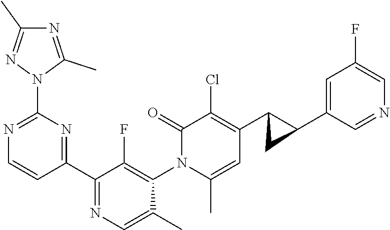 | Single known stereoisomer |
| 6.56 | 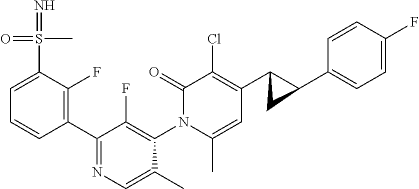 | Single unknown stereoisomer, single known atropisomer, single known cyclopropyl stereoisomer |
| 6.57 | 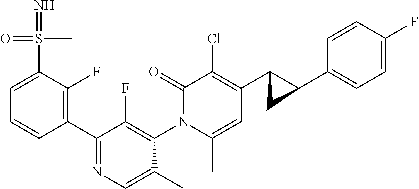 | Single unknown stereoisomer, single known atropisomer, single known cyclopropyl stereoisomer |
| 6.58 | 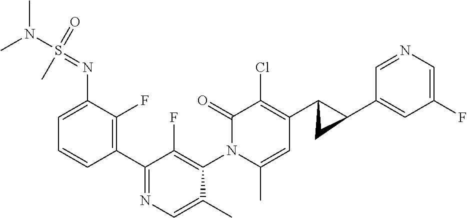 | Mixture of diastereomers; single known atropisomer, single known cyclopropyl stereoisomer |
| 6.59 | 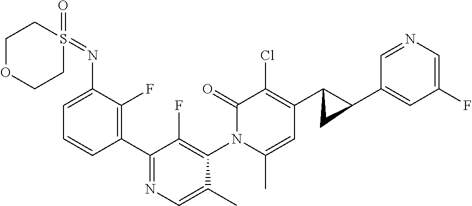 | Single known stereoisomer |
| 6.60 | 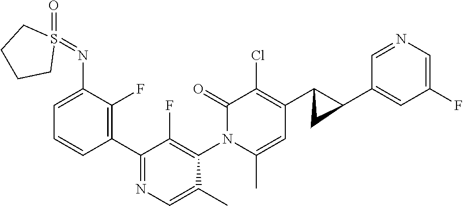 | Single known stereoisomer |
| 6.61 | 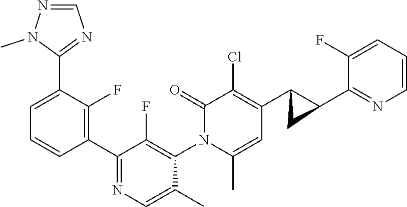 | Single known stereoisomer |
| 6.62 | 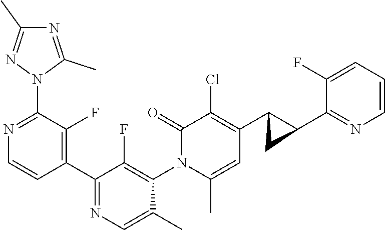 | Single known stereoisomer |
| 6.63 | 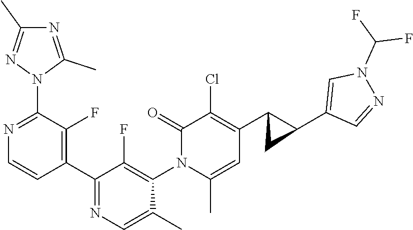 | Single known stereoisomer |
| 6.64 | 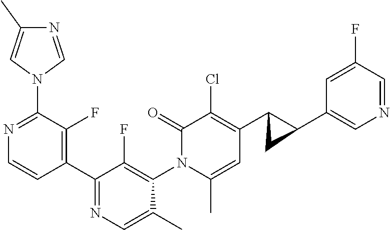 | Single known stereoisomer |
| 6.65 | 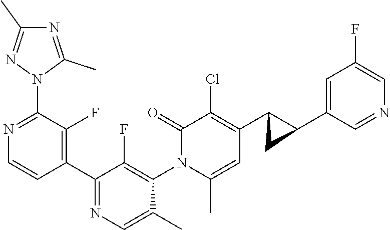 | Single known stereoisomer |
| 6.66 | 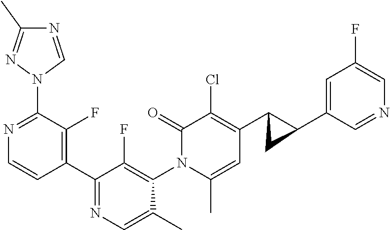 | Single known stereoisomer |
| 6.67 | 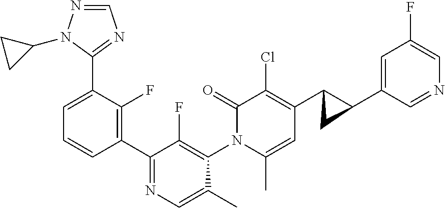 | Single known stereoisomer |
| 6.68 | 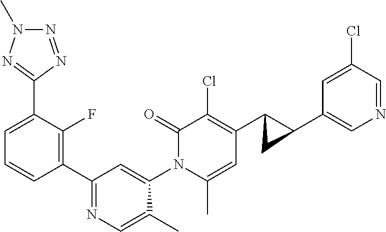 | Single known stereoisomer |
| 6.69 | 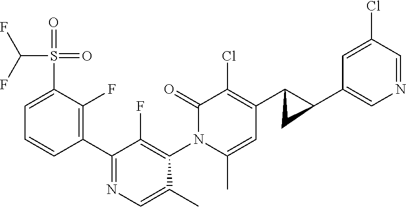 | Single known stereoisomer |
| 6.70 | 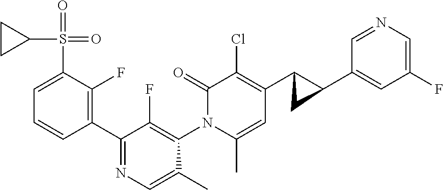 | Single known stereoisomer |
| 6.71 | 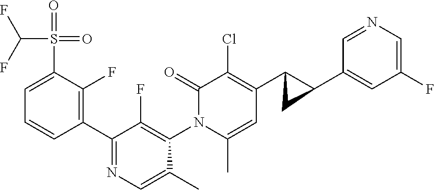 | Single known stereoisomer |
| 6.72 | 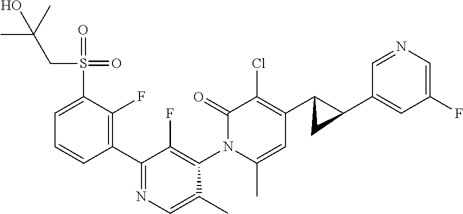 | Single known stereoisomer |
| 6.73 | 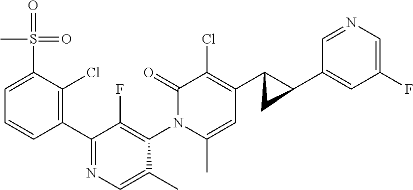 | Single known stereoisomer |
| 6.74 | 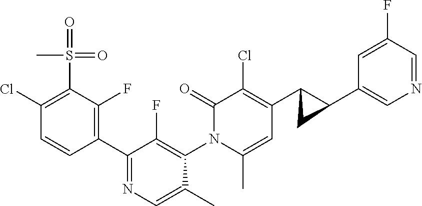 | Single known stereoisomer |
| 6.75 | 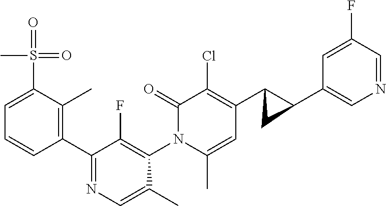 | Single known stereoisomer |
| 6.76 | 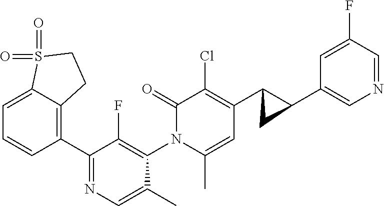 | Single known stereoisomer |
| 6.77 | 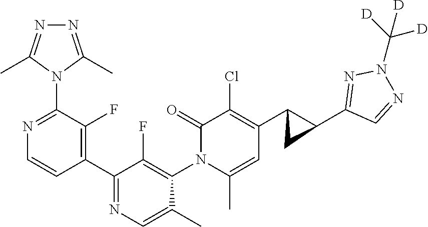 | Single known stereoisomer |
| 6.78 | 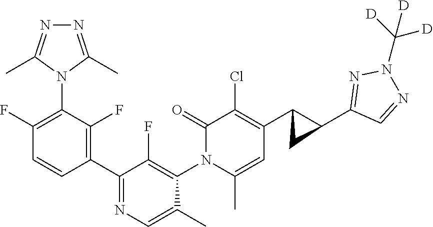 | Single known stereoisomer |
| 6.79 | 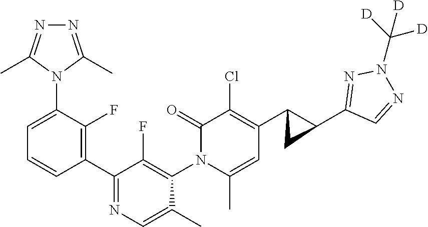 | Single known stereoisomer |
| 6.80 | 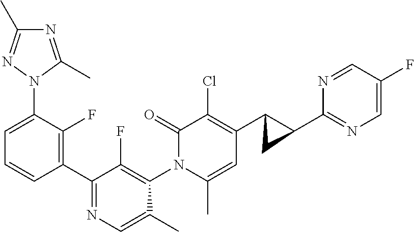 | Single known stereoisomer |
| 6.81 | 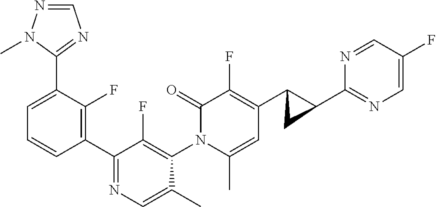 | Single known stereoisomer |
| 6.82 | 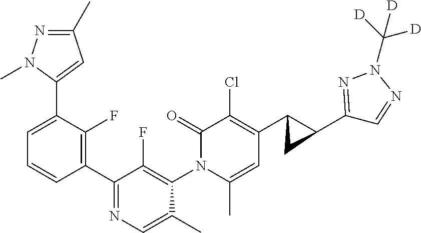 | Single known stereoisomer |
| 6.83 | 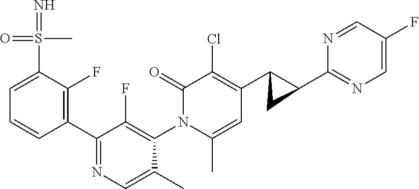 | Single unknown stereoisomer, single known atropisomer, single known cyclopropyl stereoisomer |
| 6.84 | 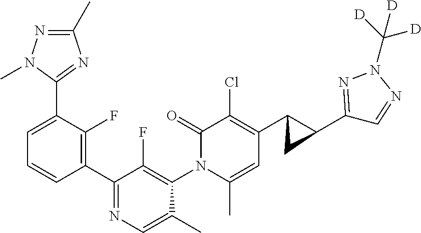 | Single known stereoisomer |
| 6.85 | 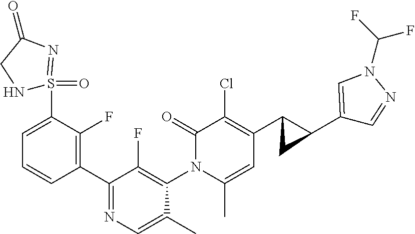 | Mixture of diastereomers |
| 6.86 | 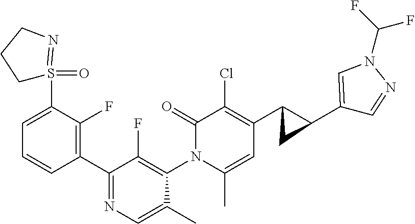 | Mixture of diastereomers, single known atropisomer, single known cyclopropyl stereoisomer |
| 6.87 | 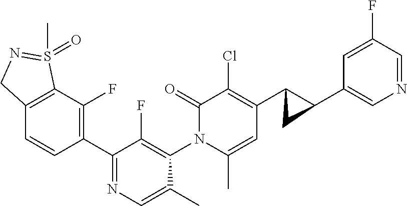 | Mixture of diastereomers, single known atropisomer, single known cyclopropyl stereoisomer |
| 7.1 | 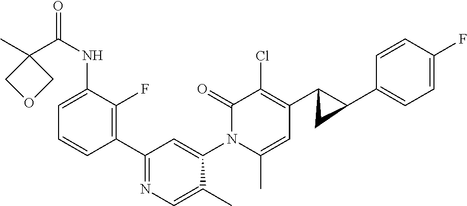 | Single known stereoisomer |
| 7.2 | 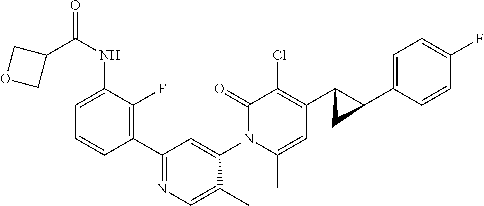 | Single known stereoisomer |
| 7.3 | 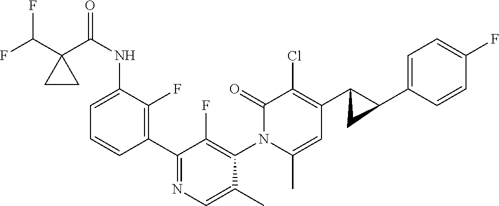 | Single known stereoisomer |
| 7.4 | 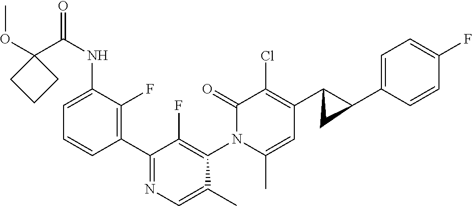 | Single known stereoisomer |
| 8.1 | 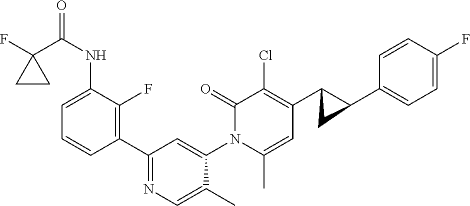 | Single known stereoisomer |
| 8.2 | 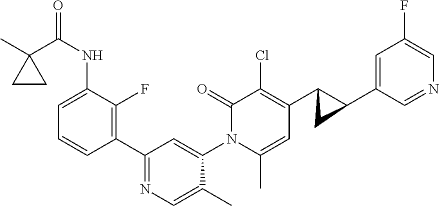 | Single known stereoisomer |
| 9.1 | 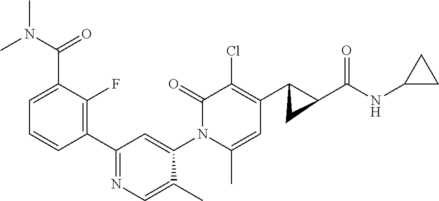 | Single known stereoisomer |
| 9.2 | 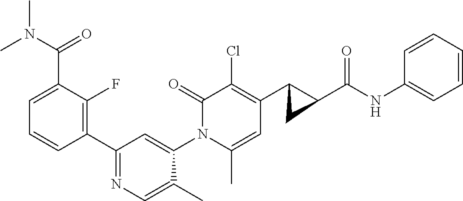 | Single known stereoisomer |
| 9.3 | 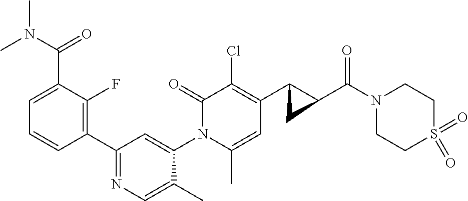 | Single known stereoisomer |
| 10.1 | 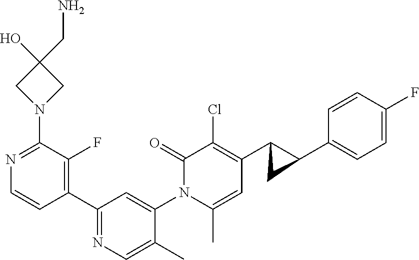 [SMILES: Cc1cnc(-c2ccnc(N3CC(O)(CN)C3)c2F)cc1-n1c(C)cc([C@H]2C[C@@H]2c2ccc(F)cc2)c(Cl)c1=O] | Single known stereoisomer |
| 10.2 | 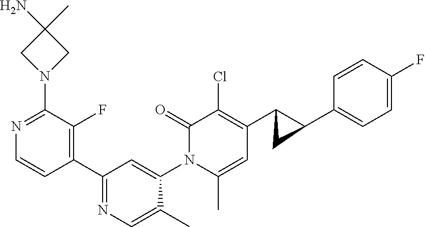 | Single known stereoisomer |
| 10.3 | 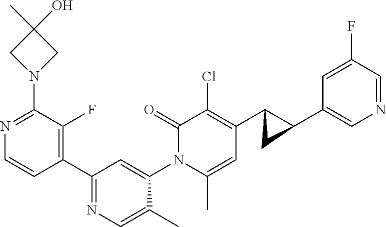 | Single known stereoisomer |
| 10.4 | 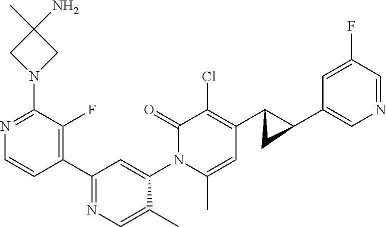 | Single known stereoisomer |
| 10.5 | 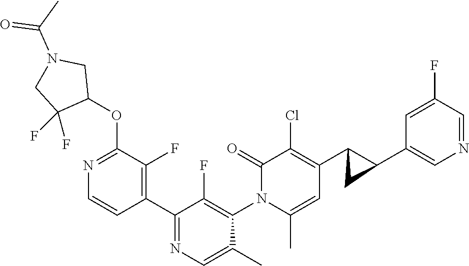 | Mixture of diastereomers, single known atropisomer, single known cyclopropyl stereoisomer |
| 10.6 | 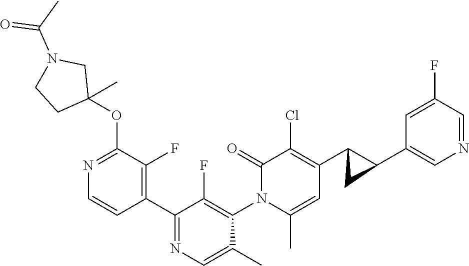 | Mixture of diastereomers, single known atropisomer, single known cyclopropyl stereoisomer |
| 10.7 | 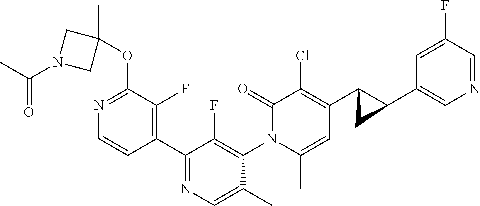 | Single known stereoisomer |
| 10.8 | 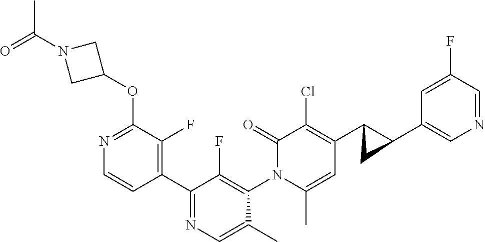 | Single known stereoisomer |
| 10.9 | 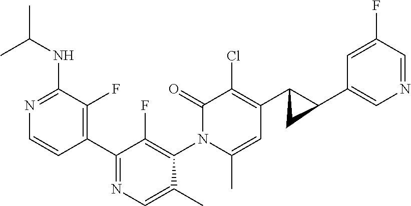 | Single known stereoisomer |
| 10.10 | 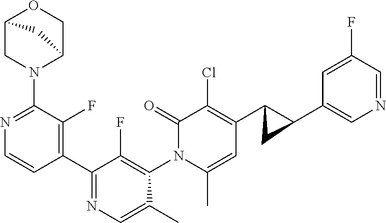 | Single known stereoisomer |
| 10.11 | 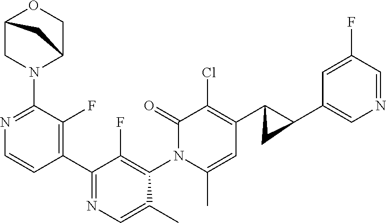 | Single known stereoisomer |
| 11.1 | 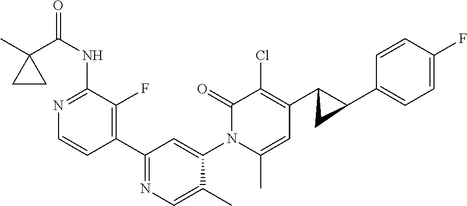 | Single known stereoisomer |
| 12.1 | 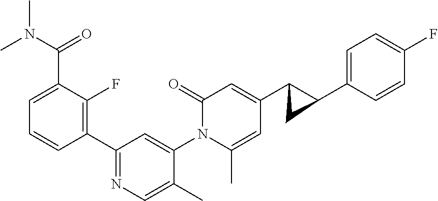 [SMILES: Cc1cnc(-c2cccc(C(=O)N(C)C)c2F)cc1-n1c(C)cc([C@H]2C[C@@H]2c2ccc(F)cc2)cc1=O] | Mixture of atropisomers, single known cyclopropyl stereoisomer |
| 13.1 | 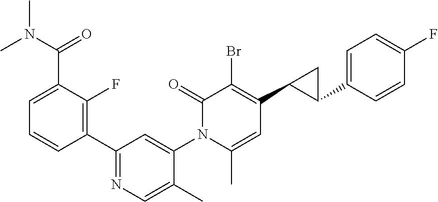 [SMILES: Cc1cnc(-c2cccc(C(=O)N(C)C)c2F)cc1-n1c(C)cc([C@H]2C[C@@H]2c2ccc(F)cc2)c(Br)c1=O] | Mixture of atropisomers, single known cyclopropyl stereoisomer |
| 14.1 | 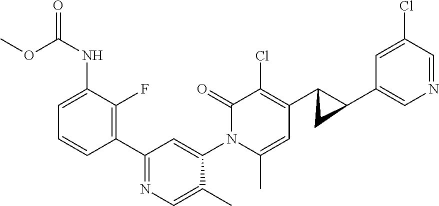 | Single known stereoisomer |
| 14.2 | 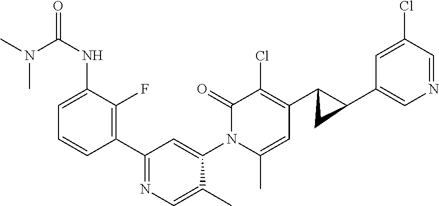 | Single known stereoisomer |
| 14.3 | 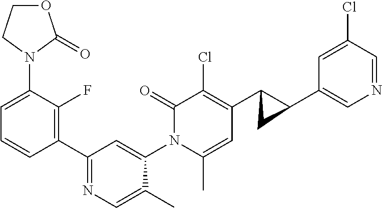 | Single known stereoisomer |
| 15.1 | 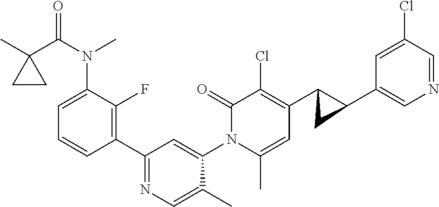 | Single known stereoisomer |
| 16.1 | 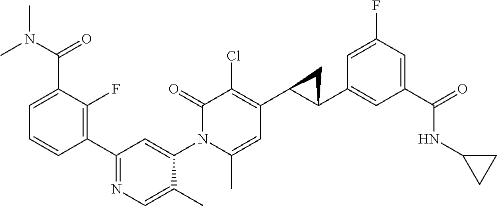 | Single known stereoisomer |
| 17.1 | 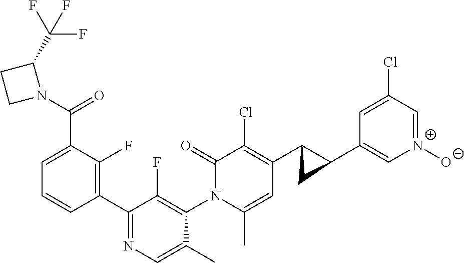 | Single known stereoisomer |
| 17.2 | 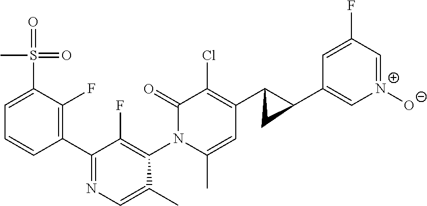 | Single known stereoisomer |
| 17.3 | 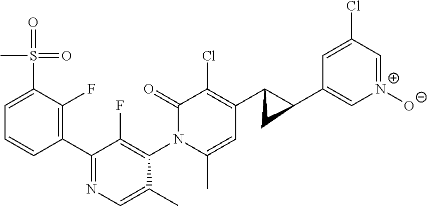 | Single known stereoisomer |
| 17.4 | 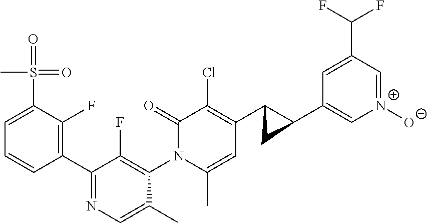 | Single known stereoisomer |
| 17.5 | 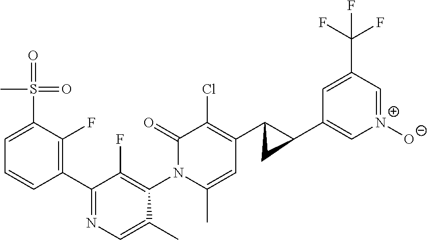 | Single known stereoisomer |
| 18.1 | 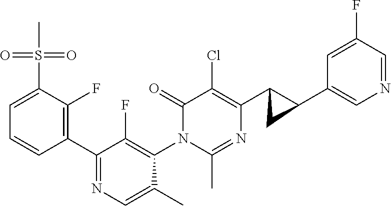 | Single known stereoisomer |
| 18.3 | 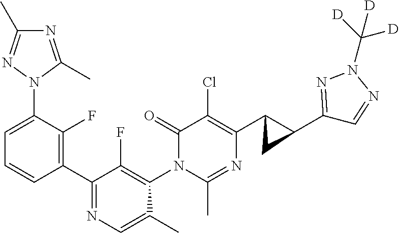 | Single known stereoisomer |
| 18.4 | 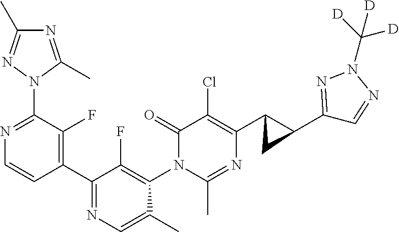 | Single known stereoisomer |
| 18.5 | 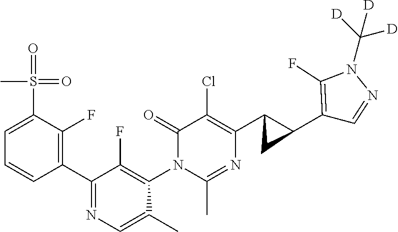 | Single known stereoisomer |
| 18.6 | 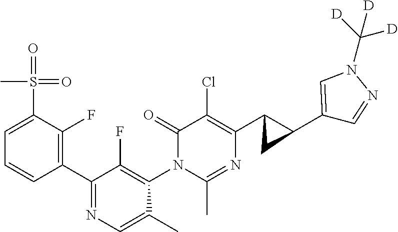 | Single known stereoisomer |
| 18.7 | 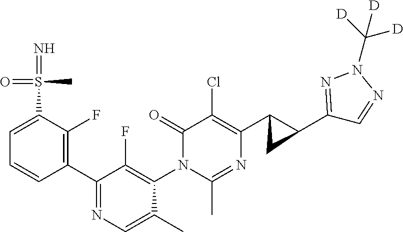 | Single known stereoisomer |
| 18.8 | 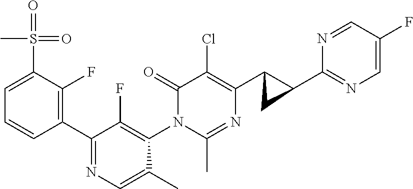 | Single known stereoisomer |
| 18.9 | 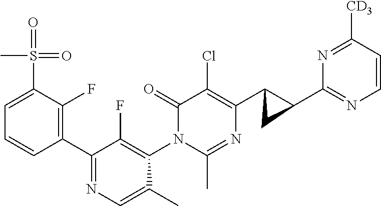 | Single known stereoisomer |
| 19.1 | 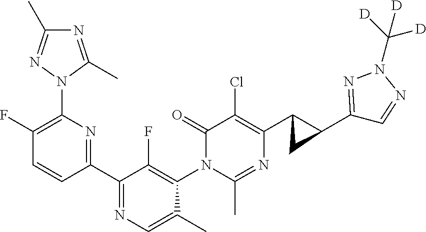 | Single known stereoisomer |
| 19.2 | 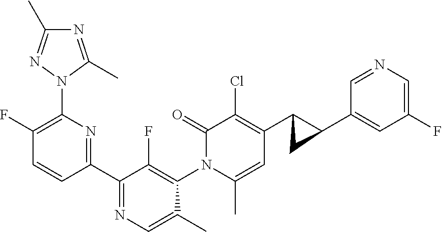 | Single known stereoisomer |
| 19.3 | 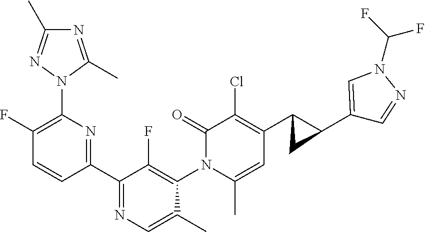 | Single known stereoisomer |
| 19.4 | 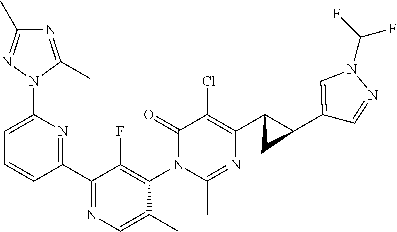 | Single known stereoisomer |
| 19.5 | 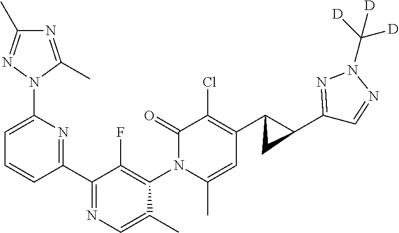 | Single known stereoisomer |
| 19.6 | 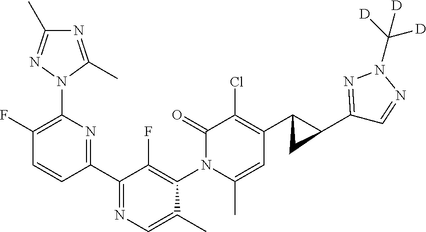 | Single known stereoisomer |
| 20.1 | 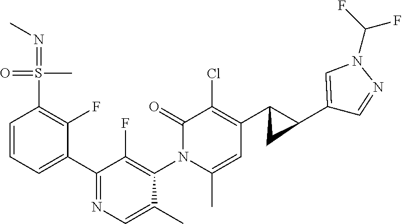 | Single unknown stereoisomer, single known atropisomer, single known cyclopropyl stereoisomer |
| 20.2 | 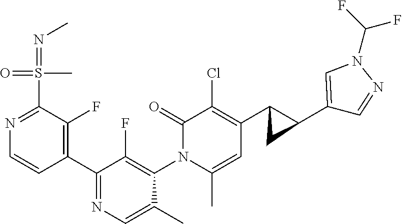 | Single unknown stereoisomer, single known atropisomer, single known cyclopropyl stereoisomer |
| 20.3 | 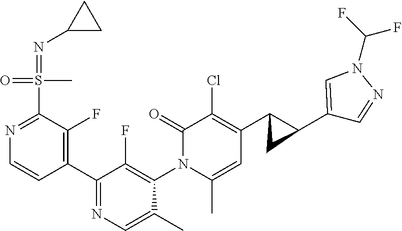 | Single unknown stereoisomer, single known atropisomer, single known cyclopropyl stereoisomer |
| 20.4 | 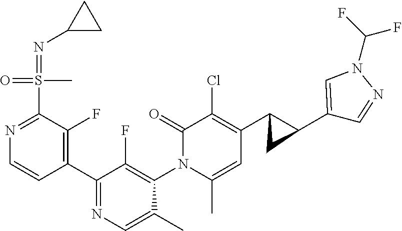 | Single unknown stereoisomer, single known atropisomer, single known cyclopropyl stereoisomer |
| 21.1 | 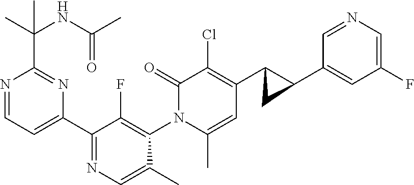 | Single known stereoisomer |
| 22.1 | 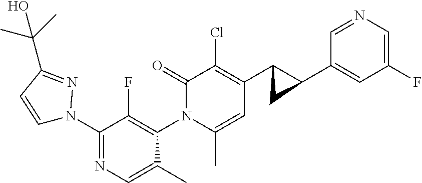 | Single known stereoisomer |

or a pharmaceutically acceptable salt thereof.

### III. Compositions and Kits

Compounds provided herein, or pharmaceutically acceptable salts thereof, are usually administered in the form of pharmaceutical compositions. Thus, provided herein are also pharmaceutical compositions that comprise one or more of the compounds provided herein or pharmaceutically acceptable salts, isomer, or a mixture thereof and one or more pharmaceutically acceptable vehicles selected from carriers, adjuvants and excipients. The compounds provided herein, or pharmaceutically acceptable salts thereof, may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In one embodiment, provided herein are pharmaceutical compositions comprising a compound provided herein (i.e., a compound of Formula I, II, III, IV, V, VI, or VII), or a pharmaceutically acceptable salt thereof, and a pharmaceutically acceptable excipient or carrier. In some embodiments, the pharmaceutical compositions comprise a therapeutically effective amount of a compound provided herein, or a pharmaceutically acceptable salt thereof, and a pharmaceutically acceptable excipient or carrier.

In some embodiments, the pharmaceutical compositions provided herein further comprise one or more (i.e., one, two, three, four; one or two; one to three; or one to four) additional therapeutic agents, or a pharmaceutically acceptable salt thereof. In some embodiments, the pharmaceutical compositions further comprise a therapeutically effective amount of the one or more (i.e., on e, two, three, four; one or two; one to three; or one to four) additional therapeutic agents, or a pharmaceutically acceptable salt thereof.

In some embodiments, the one or more additional therapeutic agents include agents that are therapeutic for an inflammatory condition. In some embodiments, the one or more additional therapeutic agents is selected from the group consisting of: veltuzumab, PF-06835375, eculizumab, milatuzumab, SM-06, SM-03, BT-063, QX-006-N, BOS-161721, AK-101, TNX-1500, theralizumab, daxdilimab, TAK-079, felzartamab, itolizumab, anifrolumab, iscalimab, dapirolizumab pegol, lanalumab, LY-3361237, JNJ-55920839, UBP-1213, DS-7011, PFI-102, BIIB-059, obexelimab, talacotuzumab, vobarilizumab, TE-2324, PRV-3279, chloroquine, hydroxychloroquine, hydroxychloroquine sulfate, COV-08-0064; GNKS-356, AVO-101, rozibafusp alfa, VRN-02, annexuzlimab, ALPN-101, bendamustine hydrochloride, BMS-986256, NKTR-35, atacicept, telitacicept, BMS-986256, M-5049, KZR-616, KPG-818, verdinexor, ALPN-303, valziflocept, LA-1, cenerimod, prednisone, corticotropin, deucravacitinib, CPL-409116, CS-12192, tofacitinib citrate, ISB-830, DV-1079, julemic acid, iberdomide, TAM-01, BML-258, brepocitinib, SDC-1801, SDC-1802, ICP-330, NTR-441, dalazatide, GSK-2646264, SKI-O-703, lanraplenib (GS-9876), GNS-1653, HMPL-523, RSLV-132, interleukin-2 follow-on biologic, interleukin-2 Anteluke, interking recombinant human interleukin-2, ILT-101, CUG-252, DZ-2002, PEGylated HLA-x (SLE), AC-0058, fenebrutinib, XNW-1011, tirabrutinib hydrochloride, branebrutinib, elsubrutinib, orelabrutinib, DWP-213388, INV-103, R-salbutamol sulphate, anchorins, NIK-SMI1, X-6, INV-17, Oshadi D, baricitinib, upadacitinib, filgotinib, itacitinib, INCB-54707, delgocitinib, DWP-212525, CKD-971, as mometasone, betamethasone, forigerimod, anandamide, DCB-SLE1, arsenic trioxide, tairuimide, TV-4710 (edratide), allogeneic human umbilical cord-derived mesenchymal stem cell therapy (hUC-MSCs), LC-200, BI-705564, SM-934, GX-101, TXR-712, TXR-711, CIT-013, MHV-370, Panzyga®, TPX-6001, TPX-7001, artenimol, and AMG-592, or a pharmaceutically acceptable salt of any of the foregoing, or any combinations thereof.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. In some embodiments, the compounds, or pharmaceutically acceptable salts thereof, and pharmaceutical compositions disclosed herein are administered by subcutaneous injection.

The pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the sterile injectable preparation disclosed herein may also be a sterile injectable solution or suspension prepared from a reconstituted lyophilized powder in a non-toxic parenterally acceptable diluent or solvent, such as a solution in 1,3-butane-diol. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. In certain embodiments the suspension is a microsuspension. In certain embodiments the suspension is a nanosuspension.

In some embodiments, formulations suitable for parenteral administration (e.g., intramuscular (IM) and subcutaneous (SC) administration) will include one or more excipients. Excipients should be compatible with the other ingredients of the formulation and physiologically innocuous to the recipient thereof. Examples of suitable excipients are well known to the person skilled in the art of parenteral formulation and may be found e.g., in Handbook of Pharmaceutical Excipients (eds. Rowe, Sheskey & Quinn), 6th edition 2009. Examples of solubilizing excipients in a parenteral formulation (e.g., an SC or IM formulation) include, but are not limited to, polysorbates (such as polysorbate 20 or 80) and poloxamers (such as poloxamer 338, 188, or 207).

In some embodiments, the compounds, or pharmaceutically acceptable salts thereof, and pharmaceutical compositions disclosed herein are administered with implants.

Oral administration may be another route for administration of the compounds provided herein or pharmaceutically acceptable salts thereof. Administration may be via, for example, capsule or enteric coated tablets. In making the pharmaceutical compositions that include at least one compound provided herein or pharmaceutically acceptable salts, isomer, or a mixture thereof, the active ingredient (such as a compound provided herein) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose or any combinations thereof. The pharmaceutical compositions can additionally include lubricating agents such as talc, magnesium stearate, and mineral oil; wetting agents; emulsifying and suspending agents; preserving agents such as methyl and propylhydroxy-benzoates; sweetening agents; and flavoring agents; or any combinations thereof.

The pharmaceutical compositions that include at least one compound described herein or pharmaceutically acceptable salts, isomer, or a mixture thereof can be formulated so as to provide quick, sustained or delayed release of the active ingredient (such as a compound provided herein) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds provided herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or pharmaceutically acceptable salts, isomer, or a mixture thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds provided herein or pharmaceutically acceptable salts thereof may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release.

A variety of materials can be used for such enteric layers or coatings, such materials including a number of polymeric acids and mixtures of polymeric acids with materials such as shellac, cetyl alcohol, and cellulose acetate.

Pharmaceutical compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In one embodiment, provided herein are kits that comprise a compound provided herein, (i.e., a compound of Formula I, II, III, IV, V, VI, or VII), or a pharmaceutically acceptable salt, stereoisomer, prodrug, or solvate thereof, and suitable packaging. In some embodiments, the kit further comprises instructions for use. In some embodiments, the kit comprises a compound provided herein (i.e., a compound of Formula I, II, III, IV, V, VI, or VII), or a pharmaceutically acceptable salt, stereoisomer, prodrug, or solvate thereof, and a label and/or instructions for use of the compounds in the treatment of the indications, including the diseases or conditions, described herein.

In some embodiments, the kits further comprise one or more (i.e., one, two, three, four; one or two; one to three; or one to four) additional therapeutic agents, or a pharmaceutically acceptable salt thereof.

In one embodiment, provided herein are articles of manufacture that comprise a compound described herein or pharmaceutically acceptable salts, isomer, or a mixture thereof in a suitable container.

In some embodiments, the container may be a vial, jar, ampoule, preloaded syringe, or intravenous bag.

### IV. Methods

The methods provided herein may be applied to cell populations in vivo or ex vivo. “In vivo” means within a living individual, as within an animal or human. In this context, the methods provided herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. Exemplary tissue samples include tumors and biopsies thereof. In this context, the present disclosure may be used for a variety of purposes, including therapeutic and experimental purposes. For example, the present disclosure may be used ex vivo to determine the optimal schedule and/or dosing of administration of a kinase inhibitor (e.g., a p38 MAP kinase inhibitor or an MK2 inhibitor) as disclosed herein for a given cell type, individual, and other parameters. Information gleaned from such use may be used for experimental purposes or in the clinic to set protocols for in vivo treatment. Other ex vivo uses for which the present disclosure may be suited are described below or will become apparent to those skilled in the art. The selected compounds may be further characterized to examine the safety or tolerance dosage in human or non-human subjects. Such properties may be examined using commonly known methods to those skilled in the art.

In one embodiment, the compounds and compositions described herein are generally useful for the inhibition of kinase activity of one or more enzymes. Examples of kinases that may be inhibited by the compounds and compositions described herein and against which the methods described herein may be useful include p38 MAP kinase, MAP kinase-activated protein kinase 2 (“MK2”), or a mutant thereof. In one embodiment, the compounds and compositions described herein are generally useful for the inhibition of MK2. MK2 is an enzyme that in humans is encoded by the MAPKAPK2 gene.

In one embodiment, provided herein is a method of inhibiting MK2 activity in a subject in need thereof, comprising administering to the subject a therapeutically effective amount of a compound provided herein (i.e., a compound of Formula I, II, III, IV, V, VI, or VII), or a pharmaceutically acceptable salt thereof, or a therapeutically effective amount of a pharmaceutical composition provided herein.

In one embodiment, provided herein is a method of treating a p38 MAP kinase-mediated disease or disorder in a subject in need thereof, comprising administering to the subject a therapeutically effective amount of a compound provided herein (i.e., a compound of Formula I, II, III, IV, V, VI, or VII), or a pharmaceutically acceptable salt thereof, or a therapeutically effective amount of a pharmaceutical composition provided herein.

In one embodiment, provided herein is a method of treating a MK2-mediated disease or disorder in a subject in need thereof, comprising administering to the subject a therapeutically effective amount of a compound provided herein (i.e., a compound of Formula I, II, III, IV, V, VI, or VII), or a pharmaceutically acceptable salt thereof, or a therapeutically effective amount of a pharmaceutical composition provided herein.

In one embodiment, provided herein is a method of treating an inflammatory condition in a subject in need thereof, comprising administering to the subject a therapeutically effective amount of a compound provided herein (i.e., a compound of Formula I, II, III, IV, V, VI, or VII), or a pharmaceutically acceptable salt thereof, or a therapeutically effective amount of a pharmaceutical composition provided herein.

In one embodiment, provided herein is a method of treating an autoimmune disorder, a chronic inflammatory disorder, an acute inflammatory disorder, an auto-inflammatory disorder, a fibrotic disorder, a metabolic disorder, a neoplastic disorder, a cardiovascular disorder, or a cerebrovascular disorder in a subject in need thereof, comprising administering to the subject a therapeutically effective amount of a compound provided herein (i.e., a compound of Formula I, II, III, IV, V, VI, or VII), or a pharmaceutically acceptable salt thereof, or a therapeutically effective amount of a pharmaceutical composition provided herein.

In one embodiment, provided herein is a method of treating rheumatoid arthritis in a subject in need thereof, comprising administering to the subject a therapeutically effective amount of a compound provided herein (i.e., a compound of Formula I, II, III, IV, V, VI, or VII), or a pharmaceutically acceptable salt thereof, or a therapeutically effective amount of a pharmaceutical composition provided herein.

Diseases or disorders associated with MK2 include autoimmune disorders, chronic inflammatory disorders, acute inflammatory disorders, auto-inflammatory disorders, fibrotic disorders, metabolic disorders, neoplastic disorders, and cardiovascular or cerebrovascular disorders.

In some embodiments, an MK2-mediated disease or disorder is an autoimmune disorder, chronic and/or acute inflammatory disorder, and/or auto-inflammatory disorder. Exemplary autoimmune and/or inflammatory and/or auto-inflammatory disorders include: inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepatic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic purpura, metal-induced autoimmunity, myasthenia gravis, pemphigus, autoimmune deafness (for example, Meniere's disease), Goodpasture's syndrome, Graves' disease, HW-related autoimmune syndromes, Guillain-Barre disease, Addison's disease, anti-phospholipid syndrome, asthma, atopic dermatitis, Celiac disease, Cushing's syndrome, dermatomyositis, idiopathic adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post-surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

In some embodiments, an MK2-mediated disease or disorder is a fibrotic disorder. Exemplary fibrotic disorders include systemic sclerosis/scleroderma, lupus nephritis, connective tissue disease, wound healing, surgical scarring, spinal cord injury, CNS scarring, acute lung injury, pulmonary fibrosis (for example, idiopathic pulmonary fibrosis or cystic fibrosis), chronic obstructive pulmonary disease, adult respiratory distress syndrome, acute lung injury, drug-induced lung injury, glomerulonephritis, chronic kidney disease (for example, diabetic nephropathy), hypertension-induced nephropathy, alimentary track or gastrointestinal fibrosis, renal fibrosis, hepatic or biliary fibrosis, liver fibrosis (for example, nonalcoholic steatohepatitis, hepatitis C, or hepatocellular carcinoma), cirrhosis (for example, primary biliary cirrhosis or cirrhosis due to fatty liver disease (for example, alcoholic and nonalcoholic steatosis)), radiation-induced fibrosis (for example, head and neck, gastrointestinal or pulmonary), primary sclerosing cholangitis, restenosis, cardiac fibrosis (for example, endomyocardial fibrosis or atrial fibrosis), ophthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, and nephrogenic systemic fibrosis.

In some embodiments, an MK2-mediated disease or disorder is a metabolic disorder. Exemplary metabolic disorders include obesity, steroid-resistance, glucose intolerance, and metabolic syndrome.

In some embodiments, an MK2-mediated disease or disorder is a neoplastic disease or disorder. Exemplary neoplastic diseases or disorders include cancers. In some embodiments, exemplary neoplastic diseases or disorders include angiogenesis disorders, multiple myeloma, leukemias (for example, acute lymphocytic leukemia, acute and chronic myelogenous leukemia, chronic lymphocytic leukemia, acute lymphoblastic leukemia, or promyelocytic leukemia), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease), myelodysplastic syndrome, fibrosarcoma, rhabdomyosarcoma; astrocytoma, neuroblastoma, glioma and schwannomas; melanoma, seminoma, teratocarcinoma, osteosarcoma, xenoderoma pigmentosum, keratoctanthoma, thyroid follicular cancer, Kaposi's sarcoma, melanoma, teratoma, rhabdomyosarcoma, metastatic and bone disorders, as well as cancer of the bone, mouth/pharynx, esophagus, larynx, stomach, intestine, colon, rectum, lung (for example, non-small cell lung cancer or small cell lung cancer), liver, pancreas, nerve, brain (for example, glioma or glioblastoma multiforme), head and neck, throat, ovary, uterus, prostate, testis, bladder, kidney, breast, gall bladder, cervix, thyroid, prostate, and skin.

In some embodiments, an MK2-mediated disorder is a cardiovascular or cerebrovascular disorder. Exemplary cardiovascular disorders include atherosclerosis, restenosis of an atherosclerotic coronary artery, acute coronary syndrome, myocardial infarction, cardiac-allograft vasculopathy and stroke. Exemplary cerebrovascular diseases include central nervous system disorders with an inflammatory or apoptotic component, Alzheimer's disease, Parkinson's disease, Huntington's disease, amyotrophic lateral sclerosis, spinal cord injury, neuronal ischemia and peripheral neuropathy.

Non-limiting examples of an inflammatory condition include, without limitation, acne, acid-induced lung injury, Addison's disease, adrenal hyperplasia, adrenocortical insufficiency, adult-onset Still's disease, adult respiratory distress syndrome (ARDS), age-related macular degeneration, aging, alcoholic hepatitis, alcoholic liver disease, allergen-induced asthma, allergic bronchopulmonary, allergic conjunctivitis, allergic contact dermatitis, allergies, allergic encephalomyelitis, allergic neuritis, allograft rejection, alopecia, alopecia areata, Alzheimer's disease, amyloidosis, amyotrophic lateral sclerosis, angina pectoris, angioedema, angiofibroma, anhidrotic ectodermal dysplasia-ill, anti-glomerular basement membrane disease, antigen-antibody complex mediated diseases, ankylosing spondylitis, antiphospholipid syndrome, aphthous stomatitis, appendicitis, arthritis, ascites, aspergillosis, asthma, atherosclerosis, atherosclerotic plaques, atopic dermatitis, atrophic thyroiditis, autoimmune diseases, autoimmune hemolytic anemia (immune pancytopenia, paroxysmal nocturnal hemoglobinuria), autoimmune polyendocrinopathies, autoimmune thrombocytopenia (idiopathic thrombocytopenic purpura, immune-mediated thrombocytopenia), autoimmune hepatitis, autoimmune thyroid disorders, autoinflammatory diseases, back pain, Bacillus anthracis infection, Bechet's disease, bee sting-induced inflammation, Behçet's syndrome, Bell's palsy, berylliosis, Blau syndrome, bone pain, bronchiolitis, bullous pemphigoid (BP) asthma, burns, bursitis, cardiac hypertrophy, carpal tunnel syndrome, Castleman's disease, catabolic disorders, cataracts, Celiac disease, cerebral aneurysm, chemical irritant-induced inflammation, chorioretinitis, chronic atypicalneutrophilic dermatosis with lipodystrophy and elevated temperature (CANDLE) syndrome, chronic heart failure, chronic lung disease of prematurity, chronic obstructive pulmonary disease (COPD), chronic pancreatitis, chronic prostatitis, chronic recurrent multifocal osteomyelitis, cicatricial alopecia, colitis, complex regional pain syndrome, complications of organ transplantation, conjunctivitis, connective tissue disease, contact dermatitis, corneal graft neovascularization, corneal ulcer, Crohn's disease, cryopyrin-associated periodic syndromes, cutaneous lupus erythematosus (CLE), cryptococcosis, cystic fibrosis, deficiency of the interleukin-1 receptor antagonist (DIRA), dermatitis, dermatitis endotoxemia, dermatomyositis, diabetic macular edema, diverticulitis, eczema, encephalitis, endometriosis, endotoxemia, eosinophilic pneumonias, epicondylitis, epidermolysis bullosa, erythema multiforme, erythroblastopenia, esophagitis, familial amyloidotic polyneuropathy, familial cold urticarial, familial Mediterranean fever, fetal growth retardation, fibromyalgia, fistulizing Crohn's disease, food allergies, giant cell arteritis, glaucoma, glioblastoma, glomerular disease, glomerular nephritis, glomerulonephritis, gluten-sensitive enteropathy, gout, gouty arthritis, graft-versus-host disease (GVHD), granulomatous hepatitis, Graves' disease, growth plate injuries, Guillain-Barre syndrome. gut diseases, hair loss, Hashimoto's thyroiditis, head injury, headache, hearing loss, heart disease, hemangioma, hemolytic anemia, hemophilic joints, Henoch-Scholein purpura, hepatitis, hereditary periodic fever syndrome, heritable disorders of connective tissue, herpes zoster and simplex, hidradenitis suppurativa (HS), hip replacement, Hodgkin's disease, Huntington's disease, hyaline membrane disease, hyperactive inflammatory response, hyperammonemia, hypercalcemia, hypercholesterolemia, hypereosinophilic syndrome (HES), hyperimmunoglobulinemia D with recurrent fever (HIDS), hypersensitivity pneumonitis, hypertropic bone formation, hypoplastic and other anemias, hypoplastic anemia, ichthyosis, idiopathic demyelinating polyneuropathy, Idiopathic inflammatory myopathies (dermatomyositis, polymyositis), idiopathic pulmonary fibrosis, idiopathic thrombocytopenic purpura, immunoglobulin nephropathies, immune complex nephritis, immune thrombocytopenic purpura (ITP), incontinentia pigmenti (IP, Bloch-Siemens syndrome), infectious mononucleosis, infectious diseases including viral diseases such as AIDS (HIV infection), hepatitis A, B, C, D, and E, herpes; inflammation, inflammation of the CNS, inflammatory bowel disease (IBD), inflammatory disease of the lower respiratory tract including bronchitis or chronic obstructive pulmonary diseases, inflammatory disease of the upper respiratory tract including the nose and sinuses such as rhinitis or sinusitis, inflammatory diseases of the respiratory tract, inflammatory ischemic event such as stroke or cardiac arrest, inflammatory lung disease, inflammatory myopathy such as myocarditis, inflammatory liver disease, inflammatory neuropathy, inflammatory pain, insect bite-induced inflammation, interstitial cystitis, interstitial lung disease, iritis, irritant-induced inflammation, ischemia/reperfusion, joint replacement, juvenile arthritis, juvenile rheumatoid arthritis, keratitis, kidney injury caused by parasitic infections, kidney transplant rejection, leptospirosis, leukocyte adhesion deficiency, lichen sclerosus (LS), Lambert-Eaton myasthenic syndrome, Loeffler's syndrome, lupus, lupus nephritis, Lyme disease, Marfan syndrome (MFS), mast cell activation syndrome, mastocytosis, meningitis, meningioma, mesothelioma, mixed connective tissue disease, Muckle-Wells syndrome (urticaria deafness amyloidosis), mucositis, multiple organ injury syndrome, multiple sclerosis, muscle wasting, muscular dystrophy, myasthenia gravis (MG), myelodysplastic syndrome, myocarditis, myositis, nasal sinusitis, necrotizing enterocolitis, neonatal onset multisystem inflammatory disease (NOMID), neovascular glaucoma, nephrotic syndrome, neuritis, neuropathological diseases, non-allergen induced asthma, obesity, ocular allergy, optic neuritis, organ transplant rejection, Osier-Weber syndrome, osteoarthritis, osteogenesis imperfecta, osteonecrosis, osteoporosis, osteoarthritis, otitis, pachyonychia congenita, Paget's disease, Paget's disease of bone, pancreatitis, Parkinson's disease, pediatric rheumatology, pelvic inflammatory disease, pemphigus, pemphigus vulgaris (PV), bullous pemphigoid (BP), pericarditis, periodic fever, periodontitis, peritoneal endometriosis, pernicious anemia (Addison's disease), pertussis, PFAPA (periodic feveraphthous pharyngitis and cervical adenopathy), pharyngitis and adenitis (PFAPA syndrome), plant irritant-induced inflammation, pneumocystis infection, pneumonia, pneumonitis, poison ivy/urushiol oil-induced inflammation, polyarthritis nodosa, polychondritis, polycystic kidney disease, polymyalgia rheumatic, giant cell arteritis, polymyositis, pouchitis, reperfusion injury and transplant rejection, primary biliary cirrhosis, primary pulmonary hypertension, primary sclerosing cholangitis (PSC), proctitis, psoriasis, psoriasis vulgaris, psoriatic arthritis, psoriatic epidermis, psychosocial stress diseases, pulmonary disease, pulmonary fibrosis, pulmonary hypertension, pyoderma gangrenosum, pyogenic granuloma retrolental fibroplasias, pyogenic sterile arthritis, Raynaud's syndrome, Reiter's disease, reactive arthritis, renal disease, renal graft rejection, reperfusion injury, respiratory distress syndrome, retinal disease, retrolental fibroplasia, Reynaud's syndrome, rheumatic carditis, rheumatic diseases, rheumatic fever, rheumatoid arthritis, rhinitis, rhinitis psoriasis, rosacea, sarcoidosis, Schnitzler syndrome, scleritis, sclerosis, scleroderma, scoliosis, seborrhea, sepsis, septic shock, severe pain, Sezary syndrome, sickle cell anemia, silica-induced disease (Silicosis), Sjogren's syndrome, skin diseases, skin irritation, skin rash, skin sensitization (contact dermatitis or allergic contact dermatitis), sleep apnea, spinal cord injury, spinal stenosis, spondyloarthropathies, sports injuries, sprains and strains, Stevens-Johnson syndrome (SJS), stroke, subarachnoid hemorrhage, sunburn, synovial inflammation, systemic inflammatory response syndrome (SIRS), systemic lupus erythematosus (SLE), systemic mast cell disease (SMCD), systemic vasculitis, systemic-onset juvenile idiopathic arthritis, temporal arteritis, tendinitis, tenosynovitis, thrombocytopenia, thyroditis, thyroiditis, tissue transplant, toxoplasmosis, trachoma, transplantation rejection, traumatic brain injury, tuberculosis, tubulointerstitial nephritis, tumor necrosis factor (TNF) receptor associated periodic syndrome (TRAPS), type 1 diabetes, type 2 diabetes, complications from type 1 or type 2 diabetes, ulcerative colitis, urticaria, uterine fibroids, uveitis, uveoretinitis, vascular restenosis, vasculitis, vasculitis (NHLBI), vitiligo, Wegener's granulomatosis, and Whipple's disease.

In some embodiments, the inflammatory condition is selected from inflammatory bowel disease, psoriasis, psoriatic arthritis, rheumatoid arthritis, glomerulonephritis, mixed connective tissue disease (MCTD), dermatomyositis, polymyositis, systemic sclerosis, antineutrophil cytoplasmic antibody-associated vasculitis, anti-phospholipid syndrome, autoimmune hemolytic anemia, macrophage activation syndrome driven inflammatory anemia, IgA nephropathy, type I diabetes, non-alcoholic steatohepatitis, and Sjogren's syndrome. In some embodiments, the inflammatory condition is inflammatory bowel disease. In some embodiments, the inflammatory condition is psoriasis. In some embodiments, the inflammatory condition is psoriatic arthritis. In some embodiments, the inflammatory condition is rheumatoid arthritis. In some embodiments, the inflammatory condition is glomerulonephritis. In some embodiments, the inflammatory condition is mixed connective tissue disease (MCTD). In some embodiments, the inflammatory condition is dermatomyositis. In some embodiments, the inflammatory condition is polymyositis. In some embodiments, the inflammatory condition is systemic sclerosis. In some embodiments, the inflammatory condition is antineutrophil cytoplasmic antibody-associated vasculitis. In some embodiments, the inflammatory condition is anti-phospholipid syndrome. In some embodiments, the inflammatory condition is autoimmune hemolytic anemia. In some embodiments, the inflammatory condition is macrophage activation syndrome driven inflammatory anemia. In some embodiments, the inflammatory condition is IgA nephropathy. In some embodiments, the inflammatory condition is type I diabetes. In some embodiments, the inflammatory condition is non-alcoholic steatohepatitis. In some embodiments, the inflammatory condition is Sjogren's syndrome.

In one embodiment, the present disclosure provides a method of treating rheumatoid arthritis in a subject in need thereof, comprising administering to the subject a therapeutically effective amount of a compound provided herein (i.e., a compound of Formula I, II, III, IV, V, VI, or VII), or a pharmaceutically acceptable salt thereof, or a therapeutically effective amount of a pharmaceutical composition provided herein.

In some embodiments, the methods provided herein further comprise administering a therapeutically effective amount of one or more additional therapeutic agents, or a pharmaceutically acceptable salt thereof.

In some embodiments, the additional therapeutic agent is selected from the group consisting of anti-inflammatory drugs, anti-atherosclerotic drugs, immunosuppressive drugs, immunomodulatory drugs, cytostatic drugs, anti-proliferative agents, angiogenesis inhibitors, kinase inhibitors, cytokine blockers, and inhibitors of cell adhesion molecules.

In some embodiments, the additional therapeutic agent is selected from the group consisting of NSAIDs, immunosuppressive drugs, immunomodulatory drugs, cytostatic drugs, antiproliferative agents, angiogenesis inhibitors, biological agents, steroids, vitamin D3 analogs, retinoids, other kinase inhibitors, cytokine blockers, corticosteroids, and inhibitors of cell adhesion molecules. In some embodiments, the additional therapeutic agent is selected from the group consisting of torcetrapib, aspirin, niacin, HMG CoA reductase inhibitors (e.g., atorvastatin, fluvastatin, lovastatin, pravastatin, rosuvastatin and simvastatin), colesevelam, cholestyramine, colestipol, gemfibrozil, probucol, and clofibrate.

In some embodiments, the additional therapeutic agent is selected from the group consisting of corticosteroids, nonsteroidal anti-inflammatory drugs (NSAID) (e.g. ibuprofen, naproxen, acetaminophen, aspirin, Fenoprofen (Nalfon), Flurbiprofen (Ansaid), Ketoprofen, Oxaprozin (Daypro), Diclofenac sodium (Voltaren), Diclofenac potassium (Cataflam), Etodolac (Lodine), Indomethacin (Indocin), Ketorolac (Toradol), Sulindac (Clinoril), Tolmetin (Tolectin), Meclofenamate (Meclomen), Mefenamic acid (Ponstel), Nabumetone (Relafen), Piroxicam (Feldene), cox-2 inhibitors (e.g., celecoxib (Celebrex))), immunosuppressants (e.g., methotrexate (Rheumatrex), leflunomide (Arava), azathioprine (Imuran), cyclosporine (Neoral, Sandimmune), tacrolimus and cyclophosphamide (Cytoxan), CD20 blockers (Rituximab), Tumor Necrosis Factor (TNF) blockers (e.g., etanercept (Enbrel), infliximab (Remicade) and adalimumab (Humira)), Abatacept (CTLA4-Ig) and interleukin-1 receptor antagonists (e.g. Anakinra (Kineret), interleukin 6 inhibitors (e.g., Actemra), interleukin 17 inhibitors (e.g., AIN457), Janus kinase inhibitors (e.g., Tasocitinib), syk inhibitors (e.g. R788), and chloroquine and its derivatives.

In some embodiments, the additional therapeutic agent is selected from the group consisting of an EGFR kinase inhibitor, MEK inhibitor, VEGFR inhibitor, anti-VEGFR2 antibody, KDR antibody, AKT inhibitor, PDK-1 inhibitor, PI3K inhibitor, c-kit/Kdr tyrosine kinase inhibitor, Bcr-Abl tyrosine kinase inhibitor, VEGFR2 inhibitor, PDGFR-beta inhibitor, KIT inhibitor, Flt3 tyrosine kinase inhibitor, PDGF receptor family inhibitor, Flt3 tyrosine kinase inhibitor, RET tyrosine kinase receptor family inhibitor, VEGF-3 receptor antagonist, Raf protein kinase family inhibitor, angiogenesis inhibitor, Erb2 inhibitor, mTOR inhibitor, IGF-1R antibody, NFkB inhibitor, proteosome inhibitor, chemotherapy agent, and glucose reduction agent.

In some embodiments, the one or more additional therapeutic agents is selected from the group consisting of veltuzumab, PF-06835375, eculizumab, milatuzumab, SM-06, SM-03, BT-063, QX-006-N, BOS-161721, AK-101, TNX-1500, theralizumab, daxdilimab, TAK-079, felzartamab, itolizumab, anifrolumab, iscalimab, dapirolizumab pegol, lanalumab, LY-3361237, JNJ-55920839, UBP-1213, DS-7011, PFI-102, BIIB-059, obexelimab, talacotuzumab, vobarilizumab, TE-2324, PRV-3279, chloroquine, hydroxychloroquine, hydroxychloroquine sulfate, COV-08-0064; GNKS-356, AVO-101, rozibafusp alfa, VRN-02, annexuzlimab, ALPN-101, bendamustine hydrochloride, BMS-986256, NKTR-35, atacicept, telitacicept, BMS-986256, M-5049, KZR-616, KPG-818, verdinexor, ALPN-303, valziflocept, LA-1, cenerimod, prednisone, corticotropin, deucravacitinib, CPL-409116, CS-12192, tofacitinib citrate, ISB-830, DV-1079, julemic acid, iberdomide, TAM-01, BML-258, brepocitinib, SDC-1801, SDC-1802, ICP-330, NTR-441, dalazatide, GSK-2646264, SKI-O-703, lanraplenib (GS-9876), GNS-1653, HMPL-523, RSLV-132, interleukin-2 follow-on biologic, interleukin-2 Anteluke, interking recombinant human interleukin-2, ILT-101, CUG-252, DZ-2002, PEGylated HLA-x (SLE), AC-0058, fenebrutinib, XNW-1011, tirabrutinib hydrochloride, branebrutinib, elsubrutinib, orelabrutinib, DWP-213388, INV-103, R-salbutamol sulphate, anchorins, NIK-SMI1, X-6, INV-17, Oshadi D, baricitinib, upadacitinib, filgotinib, itacitinib, INCB-54707, delgocitinib, DWP-212525, CKD-971, as mometasone, betamethasone, forigerimod, anandamide, DCB-SLE1, arsenic trioxide, tairuimide, TV-4710 (edratide), allogeneic human umbilical cord-derived mesenchymal stem cell therapy (hUC-MSCs), LC-200, BI-705564, SM-934, GX-101, TXR-712, TXR-711, CIT-013, MHV-370, Panzyga®, TPX-6001, TPX-7001, artenimol, and AMG-592, or a pharmaceutically acceptable salt of any of the foregoing, or any combination thereof.

In some embodiments of the methods provided herein, the subject is a human.

In some embodiments, the methods provided herein comprise administering a therapeutically effective amount of a compound provided herein (i.e., a compound of Formula I, II, III, IV, V, VI, or VII), or a pharmaceutically acceptable salt thereof. In some embodiments, the methods provided herein comprise administering a therapeutically effective amount of a pharmaceutical composition provided herein.

In one embodiment, the present disclosure provides a compound provided herein (i.e., a compound of Formula I, II, III, IV, V, VI, or VII), or a pharmaceutically acceptable salt thereof, or a pharmaceutical composition provided herein for use in therapy.

In one embodiment, provided herein is a compound provided herein (i.e., a compound of Formula I, II, III, IV, V, VI, or VII), or a pharmaceutically acceptable salt thereof, or a pharmaceutical composition provided herein for use in therapy.

In one embodiment, provided herein is a compound provided herein (i.e., a compound of Formula I, II, III, IV, V, VI, or VII), or a pharmaceutically acceptable salt thereof, or a pharmaceutical composition provided herein for use in a method of inhibiting p38 MAP kinase activity in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein (i.e., a compound of Formula I, II, III, IV, V, VI, or VII), or a pharmaceutically acceptable salt thereof, or a pharmaceutical composition provided herein for use in a method of inhibiting MK2 activity in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein (i.e., a compound of Formula I, II, III, IV, V, VI, or VII), or a pharmaceutically acceptable salt thereof, or a pharmaceutical composition provided herein for use in a method of treating a p38 MAP kinase-mediated in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein (i.e., a compound of Formula I, II, III, IV, V, VI, or VII), or a pharmaceutically acceptable salt thereof, or a pharmaceutical composition provided herein for use in a method of treating a MI 2-mediated disease or disorder in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein (i.e., a compound of Formula I, II, III, IV, V, VI, or VII), or a pharmaceutically acceptable salt thereof, or a pharmaceutical composition provided herein for use in a method of treating an inflammatory condition in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein (i.e., a compound of Formula I, II, III, IV, V, VI, or VII), or a pharmaceutically acceptable salt thereof, or a pharmaceutical composition provided herein for use in a method of treating an autoimmune disorder, a chronic inflammatory disorder, an acute inflammatory disorder, an auto-inflammatory disorder, a fibrotic disorder, a metabolic disorder, a neoplastic disorder, a cardiovascular disorder, or a cerebrovascular disorder in a subject in need thereof, comprising administering to the subject a therapeutically effective amount of the compound, or a pharmaceutically acceptable salt thereof, or a therapeutically effective amount of the pharmaceutical composition.

In one embodiment, provided herein is a compound provided herein (i.e., a compound of Formula I, II, III, IV, V, VI, or VII), or a pharmaceutically acceptable salt thereof, or a pharmaceutical composition provided herein for use in a method of treating rheumatoid arthritis in a subject in need thereof, comprising administering to the subject a therapeutically effective amount of the compound, or a pharmaceutically acceptable salt thereof, or a therapeutically effective amount of the pharmaceutical composition.

Diseases or disorders associated with MK2 include autoimmune disorders, chronic inflammatory disorders, acute inflammatory disorders, auto-inflammatory disorders, fibrotic disorders, metabolic disorders, neoplastic disorders, and cardiovascular or cerebrovascular disorders.

In some embodiments, an MK2-mediated disease or disorder is an autoimmune disorder, chronic and/or acute inflammatory disorder, and/or auto-inflammatory disorder. Exemplary autoimmune and/or inflammatory and/or auto-inflammatory disorders include: inflammatory bowel diseases (for example, ulcerative colitis or Crohn's disease), multiple sclerosis, psoriasis, arthritis, rheumatoid arthritis, osteoarthritis, juvenile arthritis, psoriatic arthritis, reactive arthritis, ankylosing spondylitis, cryopyrin associated periodic syndromes, Muckle-Wells syndrome, familial cold auto-inflammatory syndrome, neonatal-onset multisystem inflammatory disease, TNF receptor associated periodic syndrome, acute and chronic pancreatitis, atherosclerosis, gout, ankylosing spondylitis, fibrotic disorders (for example, hepalic fibrosis or idiopathic pulmonary fibrosis), nephropathy, sarcoidosis, scleroderma, anaphylaxis, diabetes (for example, diabetes mellitus type 1 or diabetes mellitus type 2), diabetic retinopathy, Still's disease, vasculitis, sarcoidosis, pulmonary inflammation, acute respiratory distress syndrome, wet and dry age-related macular degeneration, autoimmune hemolytic syndromes, autoimmune and inflammatory hepatitis, autoimmune neuropathy, autoimmune ovarian failure, autoimmune orchitis, autoimmune thrombocytopenia, silicone implant associated autoimmune disease, Sjogren's syndrome, familial Mediterranean fever, systemic lupus erythematosus, vasculitis syndromes (for example, temporal, Takayasu's and giant cell arteritis, Behçet's disease or Wegener's granulomatosis), vitiligo, secondary hematologic manifestation of autoimmune diseases (for example, anemias), drug-induced autoimmunity, Hashimoto's thyroiditis, hypophysitis, idiopathic thrombocytic purpura, metal-induced autoimmunity, myasthenia gravis, pemphigus, autoimmune deafness (for example, Meniere's disease), Goodpasture's syndrome, Graves' disease, HW-related autoimmune syndromes, Guillain-Barre disease, Addison's disease, anti-phospholipid syndrome, asthma, atopic dermatitis, Celiac disease, Cushing's syndrome, dermatomyositis, idiopathic adrenal atrophy, idiopathic thrombocytopenia, Kawasaki syndrome, Lambert-Eaton Syndrome, pernicious anemia, pollinosis, polyarteritis nodosa, primary biliary cirrhosis, primary sclerosing cholangitis, Raynaud's, Reiter's Syndrome, relapsing polychondritis, Schmidt's syndrome, thyrotoxidosis, sepsis, septic shock, endotoxic shock, exotoxin-induced toxic shock, gram negative sepsis, toxic shock syndrome, glomerulonephritis, peritonitis, interstitial cystitis, hyperoxia-induced inflammations, chronic obstructive pulmonary disease (COPD), vasculitis, graft vs. host reaction (for example, graft vs. host disease), allograft rejections (for example, acute allograft rejection or chronic allograft rejection), early transplantation rejection (for example, acute allograft rejection), reperfusion injury, pain (for example, acute pain, chronic pain, neuropathic pain, or fibromyalgia), chronic infections, meningitis, encephalitis, myocarditis, gingivitis, post-surgical trauma, tissue injury, traumatic brain injury, enterocolitis, sinusitis, uveitis, ocular inflammation, optic neuritis, gastric ulcers, esophagitis, peritonitis, periodontitis, dermatomyositis, gastritis, myositis, polymyalgia, pneumonia and bronchitis.

In some embodiments, an MK2-mediated disease or disorder is a fibrotic disorder. Exemplary fibrotic disorders include systemic sclerosis/scleroderma, lupus nephritis, connective tissue disease, wound healing, surgical scarring, spinal cord injury, CNS scarring, acute lung injury, pulmonary fibrosis (for example, idiopathic pulmonary fibrosis or cystic fibrosis), chronic obstructive pulmonary disease, adult respiratory distress syndrome, acute lung injury, drug-induced lung injury, glomerulonephritis, chronic kidney disease (for example, diabetic nephropathy), hypertension-induced nephropathy, alimentary track or gastrointestinal fibrosis, renal fibrosis, hepatic or biliary fibrosis, liver fibrosis (for example, nonalcoholic steatohepatitis, hepatitis C, or hepatocellular carcinoma), cirrhosis (for example, primary biliary cirrhosis or cirrhosis due to fatty liver disease (for example, alcoholic and nonalcoholic steatosis)), radiation-induced fibrosis (for example, head and neck, gastrointestinal or pulmonary), primary sclerosing cholangitis, restenosis, cardiac fibrosis (for example, endomyocardial fibrosis or atrial fibrosis), ophthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, and nephrogenic systemic fibrosis.

In some embodiments, an MK2-mediated disease or disorder is a metabolic disorder. Exemplary metabolic disorders include obesity, steroid-resistance, glucose intolerance, and metabolic syndrome.

In some embodiments, an MK2-mediated disease or disorder is a neoplastic disease or disorder. Exemplary neoplastic diseases or disorders include cancers. In some embodiments, exemplary neoplastic diseases or disorders include angiogenesis disorders, multiple myeloma, leukemias (for example, acute lymphocytic leukemia, acute and chronic myelogenous leukemia, chronic lymphocytic leukemia, acute lymphoblastic leukemia, or promyelocytic leukemia), lymphomas (for example, B-cell lymphoma, T-cell lymphoma, mantle cell lymphoma, hairy cell lymphoma, Burkitt's lymphoma, mast cell tumors, Hodgkin's disease or non-Hodgkin's disease), myelodysplastic syndrome, fibrosarcoma, rhabdomyosarcoma; astrocytoma, neuroblastoma, glioma and schwannomas; melanoma, seminoma, teratocarcinoma, osteosarcoma, xenoderoma pigmentosum, keratoctanthoma, thyroid follicular cancer, Kaposi's sarcoma, melanoma, teratoma, rhabdomyosarcoma, metastatic and bone disorders, as well as cancer of the bone, mouth/pharynx, esophagus, larynx, stomach, intestine, colon, rectum, lung (for example, non-small cell lung cancer or small cell lung cancer), liver, pancreas, nerve, brain (for example, glioma or glioblastoma multiforme), head and neck, throat, ovary, uterus, prostate, testis, bladder, kidney, breast, gall bladder, cervix, thyroid, prostate, and skin.

In some embodiments, an MK2-mediated disorder is a cardiovascular or cerebrovascular disorder. Exemplary cardiovascular disorders include atherosclerosis, restenosis of an atherosclerotic coronary artery, acute coronary syndrome, myocardial infarction, cardiac-allograft vasculopathy and stroke. Exemplary cerebrovascular diseases include central nervous system disorders with an inflammatory or apoptotic component, Alzheimer's disease, Parkinson's disease, Huntington's disease, amyotrophic lateral sclerosis, spinal cord injury, neuronal ischemia and peripheral neuropathy.

Non-limiting examples of an inflammatory condition include, without limitation, acne, acid-induced lung injury, Addison's disease, adrenal hyperplasia, adrenocortical insufficiency, adult-onset Still's disease, adult respiratory distress syndrome (ARDS), age-related macular degeneration, aging, alcoholic hepatitis, alcoholic liver disease, allergen-induced asthma, allergic bronchopulmonary, allergic conjunctivitis, allergic contact dermatitis, allergies, allergic encephalomyelitis, allergic neuritis, allograft rejection, alopecia, alopecia areata, Alzheimer's disease, amyloidosis, amyotrophic lateral sclerosis, angina pectoris, angioedema, angiofibroma, anhidrotic ectodermal dysplasia-ill, anti-glomerular basement membrane disease, antigen-antibody complex mediated diseases, ankylosing spondylitis, antiphospholipid syndrome, aphthous stomatitis, appendicitis, arthritis, ascites, aspergillosis, asthma, atherosclerosis, atherosclerotic plaques, atopic dermatitis, atrophic thyroiditis, autoimmune diseases, autoimmune hemolytic anemia (immune pancytopenia, paroxysmal nocturnal hemoglobinuria), autoimmune polyendocrinopathies, autoimmune thrombocytopenia (idiopathic thrombocytopenic purpura, immune-mediated thrombocytopenia), autoimmune hepatitis, autoimmune thyroid disorders, autoinflammatory diseases, back pain, Bacillus anthracis infection, Bechet's disease, bee sting-induced inflammation, Behçet's syndrome, Bell's palsy, berylliosis, Blau syndrome, bone pain, bronchiolitis, bullous pemphigoid (BP) asthma, burns, bursitis, cardiac hypertrophy, carpal tunnel syndrome, Castleman's disease, catabolic disorders, cataracts, Celiac disease, cerebral aneurysm, chemical irritant-induced inflammation, chorioretinitis, chronic atypicalneutrophilic dermatosis with lipodystrophy and elevated temperature (CANDLE) syndrome, chronic heart failure, chronic lung disease of prematurity, chronic obstructive pulmonary disease (COPD), chronic pancreatitis, chronic prostatitis, chronic recurrent multifocal osteomyelitis, cicatricial alopecia, colitis, complex regional pain syndrome, complications of organ transplantation, conjunctivitis, connective tissue disease, contact dermatitis, corneal graft neovascularization, corneal ulcer, Crohn's disease, cryopyrin-associated periodic syndromes, cutaneous lupus erythematosus (CLE), cryptococcosis, cystic fibrosis, deficiency of the interleukin-1 receptor antagonist (DIRA), dermatitis, dermatitis endotoxemia, dermatomyositis, diabetic macular edema, diverticulitis, eczema, encephalitis, endometriosis, endotoxemia, eosinophilic pneumonias, epicondylitis, epidermolysis bullosa, erythema multiforme, erythroblastopenia, esophagitis, familial amyloidotic polyneuropathy, familial cold urticarial, familial Mediterranean fever, fetal growth retardation, fibromyalgia, fistulizing Crohn's disease, food allergies, giant cell arteritis, glaucoma, glioblastoma, glomerular disease, glomerular nephritis, glomerulonephritis, gluten-sensitive enteropathy, gout, gouty arthritis, graft-versus-host disease (GVHD), granulomatous hepatitis, Graves' disease, growth plate injuries, Guillain-Barre syndrome. gut diseases, hair loss, Hashimoto's thyroiditis, head injury, headache, hearing loss, heart disease, hemangioma, hemolytic anemia, hemophilic joints, Henoch-Scholein purpura, hepatitis, hereditary periodic fever syndrome, heritable disorders of connective tissue, herpes zoster and simplex, hidradenitis suppurativa (HS), hip replacement, Hodgkin's disease, Huntington's disease, hyaline membrane disease, hyperactive inflammatory response, hyperammonemia, hypercalcemia, hypercholesterolemia, hypereosinophilic syndrome (HES), hyperimmunoglobulinemia D with recurrent fever (HIDS), hypersensitivity pneumonitis, hypertropic bone formation, hypoplastic and other anemias, hypoplastic anemia, ichthyosis, idiopathic demyelinating polyneuropathy, Idiopathic inflammatory myopathies (dermatomyositis, polymyositis), idiopathic pulmonary fibrosis, idiopathic thrombocytopenic purpura, immunoglobulin nephropathies, immune complex nephritis, immune thrombocytopenic purpura (ITP), incontinentia pigmenti (IP, Bloch-Siemens syndrome), infectious mononucleosis, infectious diseases including viral diseases such as AIDS (HIV infection), hepatitis A, B, C, D, and E, herpes; inflammation, inflammation of the CNS, inflammatory bowel disease (IBD), inflammatory disease of the lower respiratory tract including bronchitis or chronic obstructive pulmonary diseases, inflammatory disease of the upper respiratory tract including the nose and sinuses such as rhinitis or sinusitis, inflammatory diseases of the respiratory tract, inflammatory ischemic event such as stroke or cardiac arrest, inflammatory lung disease, inflammatory myopathy such as myocarditis, inflammatory liver disease, inflammatory neuropathy, inflammatory pain, insect bite-induced inflammation, interstitial cystitis, interstitial lung disease, iritis, irritant-induced inflammation, ischemia/reperfusion, joint replacement, juvenile arthritis, juvenile rheumatoid arthritis, keratitis, kidney injury caused by parasitic infections, kidney transplant rejection, leptospirosis, leukocyte adhesion deficiency, lichen sclerosus (LS), Lambert-Eaton myasthenic syndrome, Loeffler's syndrome, lupus, lupus nephritis, Lyme disease, Marfan syndrome (MFS), mast cell activation syndrome, mastocytosis, meningitis, meningioma, mesothelioma, mixed connective tissue disease, Muckle-Wells syndrome (urticaria deafness amyloidosis), mucositis, multiple organ injury syndrome, multiple sclerosis, muscle wasting, muscular dystrophy, myasthenia gravis (MG), myelodysplastic syndrome, myocarditis, myositis, nasal sinusitis, necrotizing enterocolitis, neonatal onset multisystem inflammatory disease (NOMID), neovascular glaucoma, nephrotic syndrome, neuritis, neuropathological diseases, non-allergen induced asthma, obesity, ocular allergy, optic neuritis, organ transplant rejection, Osier-Weber syndrome, osteoarthritis, osteogenesis imperfecta, osteonecrosis, osteoporosis, osteoarthritis, otitis, pachyonychia congenita, Paget's disease, Paget's disease of bone, pancreatitis, Parkinson's disease, pediatric rheumatology, pelvic inflammatory disease, pemphigus, pemphigus vulgaris (PV), bullous pemphigoid (BP), pericarditis, periodic fever, periodontitis, peritoneal endometriosis, pernicious anemia (Addison's disease), pertussis, PFAPA (periodic fever aphthous pharyngitis and cervical adenopathy), pharyngitis and adenitis (PFAPA syndrome), plant irritant-induced inflammation, pneumocystis infection, pneumonia, pneumonitis, poison ivy/urushiol oil-induced inflammation, polyarthritis nodosa, polychondritis, polycystic kidney disease, polymyalgia rheumatic, giant cell arteritis, polymyositis, pouchitis, reperfusion injury and transplant rejection, primary biliary cirrhosis, primary pulmonary hypertension, primary sclerosing cholangitis (PSC), proctitis, psoriasis, psoriasis vulgaris, psoriatic arthritis, psoriatic epidermis, psychosocial stress diseases, pulmonary disease, pulmonary fibrosis, pulmonary hypertension, pyoderma gangrenosum, pyogenic granuloma retrolental fibroplasias, pyogenic sterile arthritis, Raynaud's syndrome, Reiter's disease, reactive arthritis, renal disease, renal graft rejection, reperfusion injury, respiratory distress syndrome, retinal disease, retrolental fibroplasia, Reynaud's syndrome, rheumatic carditis, rheumatic diseases, rheumatic fever, rheumatoid arthritis, rhinitis, rhinitis psoriasis, rosacea, sarcoidosis, Schnitzler syndrome, scleritis, sclerosis, scleroderma, scoliosis, seborrhea, sepsis, septic shock, severe pain, Sezary syndrome, sickle cell anemia, silica-induced disease (Silicosis), Sjogren's syndrome, skin diseases, skin irritation, skin rash, skin sensitization (contact dermatitis or allergic contact dermatitis), sleep apnea, spinal cord injury, spinal stenosis, spondyloarthropathies, sports injuries, sprains and strains, Stevens-Johnson syndrome (SJS), stroke, subarachnoid hemorrhage, sunburn, synovial inflammation, systemic inflammatory response syndrome (SIRS), systemic lupus erythematosus (SLE), systemic mast cell disease (SMCD), systemic vasculitis, systemic-onset juvenile idiopathic arthritis, temporal arteritis, tendinitis, tenosynovitis, thrombocytopenia, thyroditis, thyroiditis, tissue transplant, toxoplasmosis, trachoma, transplantation rejection, traumatic brain injury, tuberculosis, tubulointerstitial nephritis, tumor necrosis factor (TNF) receptor associated periodic syndrome (TRAPS), type 1 diabetes, type 2 diabetes, complications from type 1 or type 2 diabetes, ulcerative colitis, urticaria, uterine fibroids, uveitis, uveoretinitis, vascular restenosis, vasculitis, vasculitis (NHLBI), vitiligo, Wegener's granulomatosis, and Whipple's disease.

In some embodiments, the inflammatory condition is selected from inflammatory bowel disease, psoriasis, psoriatic arthritis, rheumatoid arthritis, glomerulonephritis, mixed connective tissue disease (MCTD), dermatomyositis, polymyositis, systemic sclerosis, antineutrophil cytoplasmic antibody-associated vasculitis, anti-phospholipid syndrome, autoimmune hemolytic anemia, macrophage activation syndrome driven inflammatory anemia, IgA nephropathy, type I diabetes, non-alcoholic steatohepatitis, and Sjogren's syndrome. In some embodiments, the inflammatory condition is inflammatory bowel disease. In some embodiments, the inflammatory condition is psoriasis. In some embodiments, the inflammatory condition is psoriatic arthritis. In some embodiments, the inflammatory condition is rheumatoid arthritis. In some embodiments, the inflammatory condition is glomerulonephritis. In some embodiments, the inflammatory condition is mixed connective tissue disease (MCTD). In some embodiments, the inflammatory condition is dermatomyositis. In some embodiments, the inflammatory condition is polymyositis. In some embodiments, the inflammatory condition is systemic sclerosis. In some embodiments, the inflammatory condition is antineutrophil cytoplasmic antibody-associated vasculitis. In some embodiments, the inflammatory condition is anti-phospholipid syndrome. In some embodiments, the inflammatory condition is autoimmune hemolytic anemia. In some embodiments, the inflammatory condition is macrophage activation syndrome driven inflammatory anemia. In some embodiments, the inflammatory condition is IgA nephropathy. In some embodiments, the inflammatory condition is type I diabetes. In some embodiments, the inflammatory condition is non-alcoholic steatohepatitis. In some embodiments, the inflammatory condition is Sjogren's syndrome.

In one embodiment, the present disclosure provides a compound provided herein (i.e., a compound of Formula I, II, III, IV, V, VI, or VII), or a pharmaceutically acceptable salt thereof, or a pharmaceutical composition provided herein, for use in a method of treating rheumatoid arthritis in a subject in need thereof, comprising administering to the subject a therapeutically effective amount of a compound provided herein (i.e., a compound of Formula I, II, III, IV, V, VI, or VII), or a pharmaceutically acceptable salt thereof, or a therapeutically effective amount of a pharmaceutical composition provided herein.

In some embodiments, the uses provided herein further comprise administering a therapeutically effective amount of one or more additional therapeutic agents, or a pharmaceutically acceptable salt thereof.

In some embodiments, the additional therapeutic agent is selected from the group consisting of anti-inflammatory drugs, anti-atherosclerotic drugs, immunosuppressive drugs, immunomodulatory drugs, cytostatic drugs, anti-proliferative agents, angiogenesis inhibitors, kinase inhibitors, cytokine blockers, and inhibitors of cell adhesion molecules.

In some embodiments, the additional therapeutic agent is selected from the group consisting of NSAIDs, immunosuppressive drugs, immunomodulatory drugs, cytostatic drugs, antiproliferative agents, angiogenesis inhibitors, biological agents, steroids, vitamin D3 analogs, retinoids, other kinase inhibitors, cytokine blockers, corticosteroids, and inhibitors of cell adhesion molecules. In some embodiments, the additional therapeutic agent is selected from the group consisting of torcetrapib, aspirin, niacin, HMG CoA reductase inhibitors (e.g., atorvastatin, fluvastatin, lovastatin, pravastatin, rosuvastatin and simvastatin), colesevelam, cholestyramine, colestipol, gemfibrozil, probucol, and clofibrate.

In some embodiments, the additional therapeutic agent is selected from the group consisting of corticosteroids, nonsteroidal anti-inflammatory drugs (NSAID) (e.g. ibuprofen, naproxen, acetaminophen, aspirin, Fenoprofen (Nalfon), Flurbiprofen (Ansaid), Ketoprofen, Oxaprozin (Daypro), Diclofenac sodium (Voltaren), Diclofenac potassium (Cataflam), Etodolac (Lodine), Indomethacin (Indocin), Ketorolac (Toradol), Sulindac (Clinoril), Tolmetin (Tolectin), Meclofenamate (Meclomen), Mefenamic acid (Ponstel), Nabumetone (Relafen), Piroxicam (Feldene), cox-2 inhibitors (e.g., celecoxib (Celebrex))), immunosuppressants (e.g., methotrexate (Rheumatrex), leflunomide (Arava), azathioprine (Imuran), cyclosporine (Neoral, Sandimmune), tacrolimus and cyclophosphamide (Cytoxan), CD20 blockers (Rituximab), Tumor Necrosis Factor (TNF) blockers (e.g., etanercept (Enbrel), infliximab (Remicade) and adalimumab (Humira)), Abatacept (CTLA4-Ig) and interleukin-1 receptor antagonists (e.g. Anakinra (Kineret), interleukin 6 inhibitors (e.g., Actemra), interleukin 17 inhibitors (e.g., AIN457), Janus kinase inhibitors (e.g., Tasocitinib), syk inhibitors (e.g. R788), and chloroquine and its derivatives.

In some embodiments, the additional therapeutic agent is selected from the group consisting of an EGFR kinase inhibitor, MEK inhibitor, VEGFR inhibitor, anti-VEGFR2 antibody, KDR antibody, AKT inhibitor, PDK-1 inhibitor, PI3K inhibitor, c-kit/Kdr tyrosine kinase inhibitor, Bcr-Abl tyrosine kinase inhibitor, VEGFR2 inhibitor, PDGFR-beta inhibitor, KIT inhibitor, Flt3 tyrosine kinase inhibitor, PDGF receptor family inhibitor, Flt3 tyrosine kinase inhibitor, RET tyrosine kinase receptor family inhibitor, VEGF-3 receptor antagonist, Raf protein kinase family inhibitor, angiogenesis inhibitor, Erb2 inhibitor, mTOR inhibitor, IGF-1R antibody, NFkB inhibitor, proteosome inhibitor, chemotherapy agent, and glucose reduction agent.

In some embodiments, the one or more additional therapeutic agents is selected from the group consisting of veltuzumab, PF-06835375, eculizumab, milatuzumab, SM-06, SM-03, BT-063, QX-006-N, BOS-161721, AK-101, TNX-1500, theralizumab, daxdilimab, TAK-079, felzartamab, itolizumab, anifrolumab, iscalimab, dapirolizumab pegol, lanalumab, LY-3361237, JNJ-55920839, UBP-1213, DS-7011, PFI-102, BIIB-059, obexelimab, talacotuzumab, vobarilizumab, TE-2324, PRV-3279, chloroquine, hydroxychloroquine, hydroxychloroquine sulfate, COV-08-0064; GNKS-356, AVO-101, rozibafusp alfa, VRN-02, annexuzlimab, ALPN-101, bendamustine hydrochloride, BMS-986256, NKTR-35, atacicept, telitacicept, BMS-986256, M-5049, KZR-616, KPG-818, verdinexor, ALPN-303, valziflocept, LA-1, cenerimod, prednisone, corticotropin, deucravacitinib, CPL-409116, CS-12192, tofacitinib citrate, ISB-830, DV-1079, julemic acid, iberdomide, TAM-01, BML-258, brepocitinib, SDC-1801, SDC-1802, ICP-330, NTR-441, dalazatide, GSK-2646264, SKI-O-703, lanraplenib (GS-9876), GNS-1653, HMPL-523, RSLV-132, interleukin-2 follow-on biologic, interleukin-2 Anteluke, interking recombinant human interleukin-2, ILT-101, CUG-252, DZ-2002, PEGylated HLA-x (SLE), AC-0058, fenebrutinib, XNW-1011, tirabrutinib hydrochloride, branebrutinib, elsubrutinib, orelabrutinib, DWP-213388, INV-103, R-salbutamol sulphate, anchorins, NIK-SMI1, X-6, INV-17, Oshadi D, baricitinib, upadacitinib, filgotinib, itacitinib, INCB-54707, delgocitinib, DWP-212525, CKD-971, as mometasone, betamethasone, forigerimod, anandamide, DCB-SLE1, arsenic trioxide, tairuimide, TV-4710 (edratide), allogeneic human umbilical cord-derived mesenchymal stem cell therapy (hUC-MSCs), LC-200, BI-705564, SM-934, GX-101, TXR-712, TXR-711, CIT-013, MHV-370, Panzyga®, TPX-6001, TPX-7001, artenimol, and AMG-592, or a pharmaceutically acceptable salt of any of the foregoing, or any combination thereof.

In some embodiments of the uses provided herein, the subject is a human.

In some embodiments, the uses provided herein comprise administering a therapeutically effective amount of a compound provided herein (i.e., a compound of Formula I, II, III, IV, V, VI, or VII), or a pharmaceutically acceptable salt thereof. In some embodiments, the methods provided herein comprise administering a therapeutically effective amount of a pharmaceutical composition provided herein.

### V. Administration

The compounds of the present disclosure or pharmaceutically acceptable salts thereof (also referred to herein as the active ingredients) can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with, for example, the condition of the recipient. An advantage of certain compounds disclosed herein, or pharmaceutically acceptable salts thereof, is that they are orally bioavailable and can be dosed orally.

A compound of the present disclosure, or a pharmaceutically acceptable salt thereof, may be administered to an individual in accordance with an effective dosing regimen for a desired period of time or duration, such as at least about one month, at least about 2 months, at least about 3 months, at least about 6 months, or at least about 12 months or longer. In some embodiments, the compound, or a pharmaceutically acceptable salt thereof, is administered on a daily or intermittent schedule for the duration of the individual's life.

The specific dose level of a compound of the present disclosure, or a pharmaceutically acceptable salt thereof, for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound provided herein, or a pharmaceutically acceptable salt thereof, per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.25 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein, or a pharmaceutically acceptable salt thereof, administered per dose or per day. Daily dosage of a compound of Formula I, II, III, IV, V, VI, or VII, or a pharmaceutically acceptable salt or pharmaceutically acceptable tautomer thereof, may be from about 1 mg to 4,000 mg, from about 2,000 to 4,000 mg/day, from about 1 to 2,000 mg/day, from about 1 to 1,000 mg/day, from about 10 to 500 mg/day, from about 20 to 500 mg/day, from about 10 to 300 mg/day, from about 10 to 200 mg/day, or from about 10 to 150 mg/day.

The dosage or dosing frequency of a compound of the present disclosure, or a pharmaceutically acceptable salt thereof, may be adjusted over the course of the treatment, based on the judgment of the administering physician.

The compounds of the present disclosure, or pharmaceutically acceptable salts thereof, may be administered to an individual (e.g., a human) in a therapeutically effective amount. In some embodiments, the compound, or a pharmaceutically acceptable salt thereof, is administered once daily.

The compounds provided herein, or pharmaceutically acceptable salts thereof, can be administered by any useful route and means, such as by oral or parenteral (e.g., intravenous) administration. Therapeutically effective amounts of the compound, or a pharmaceutically acceptable salt thereof, may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein, or pharmaceutically acceptable salts thereof, include from about 0.2 mg to about 30 mg per day, or from about 5 mg to about 300 mg per day, or from about 0.2 μg to about 30 mg per day, or from about 5 μg to about 300 μg per day.

A compound of the present disclosure, or a pharmaceutically acceptable salt thereof, may be combined with one or more additional therapeutic agents in any dosage amount of the compound of the present disclosure or a pharmaceutically acceptable salt thereof (e.g., from 1 mg to 1000 mg of compound). Therapeutically effective amounts may include from about 0.1 mg per dose to about 1000 mg per dose, such as from about 5 mg per dose to about 500 mg per dose, or such as from about 5 mg per dose to about 400 mg per dose, or such as from about 10 mg per dose to about 350 mg per dose, or such as from about 10 mg per dose to about 300 mg per dose. Other therapeutically effective amounts of the compound of Formula I, II, III, IV, V, VI, or VII, or a pharmaceutically acceptable salt thereof, are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the compound of the present disclosure, or pharmaceutically acceptable salts thereof, are about 100, 125, 150, 175, 200, 225, 250, 275, 300, 325, 350,375, 400, 425, 450, 475, 500,525, 550, 575, 600, 625,650, 675, 700, 725, 750, 775, 800, 825, 850, 875, 900, 925, 950, 975, or about 1000 mg per dose.

In some embodiments, a therapeutically effective amount of the compound of Formula I, II, III, IV, V, VI, or VII, or a pharmaceutically acceptable salt thereof, is about 1 mg to about 600 mg. In some embodiments, a therapeutically effective amount of the compound of Formula I, II, III, IV, V, VI, or VII, or a pharmaceutically acceptable salt thereof, is about 1 mg to about 500 mg. In some embodiments, a therapeutically effective amount of the compound of Formula I, II, III, IV, V, VI, or VII, or a pharmaceutically acceptable salt thereof, is about 1 mg to about 400 mg. In some embodiments, a therapeutically effective amount of the compound of Formula I, II, III, IV, V, VI, or VII, or a pharmaceutically acceptable salt thereof, is about 1 mg to about 300 mg. In some embodiments, a therapeutically effective amount of the compound of Formula I, II, III, IV, V, VI, or VII, or a pharmaceutically acceptable salt thereof, is about 1 mg to about 200 mg. In some embodiments, a therapeutically effective amount of the compound of Formula I, II, III, IV, V, VI, or VII, or a pharmaceutically acceptable salt thereof, is about 1 mg to about 100 mg. In some embodiments, a therapeutically effective amount of the compound of Formula I, II, III, IV, V, VI, or VII, or a pharmaceutically acceptable salt thereof, is about 1 mg to about 75 mg. In some embodiments, a therapeutically effective amount of the compound of Formula I, II, III, IV, V, VI, or VII, or a pharmaceutically acceptable salt thereof, is about 1 mg to about 50 mg. In some embodiments, a therapeutically effective amount of the compound of Formula I, II, III, IV, V, VI, or VII, or a pharmaceutically acceptable salt thereof, is about 1 mg to about 25 mg. In some embodiments, a therapeutically effective amount of the compound of Formula I, II, III, IV, V, VI, or VII, or a pharmaceutically acceptable salt thereof, is about 1 mg to about 20 mg. In some embodiments, a therapeutically effective amount of the compound of Formula I, II, III, IV, V, VI, or VII, or a pharmaceutically acceptable salt thereof, is about 1 mg to about 15 mg. In some embodiments, a therapeutically effective amount of the compound of Formula I, II, III, IV, V, VI, or VII, or a pharmaceutically acceptable salt thereof, is about 1 mg to about 10 mg. In some embodiments, a therapeutically effective amount of the compound of Formula I, II, III, IV, V, VI, or VII, or a pharmaceutically acceptable salt thereof, is about 1 mg to about 5 mg. In some embodiments, a therapeutically effective amount of the compound of Formula I, II, III, IV, V, VI, or VII, or a pharmaceutically acceptable salt thereof, is about 1 mg, about 2 mg, about 3 mg, about 4 mg, about 5 mg about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, or about 25 mg. In some embodiments, a therapeutically effective amount of the compound of Formula I, II, III, IV, V, VI, or VII, or a pharmaceutically acceptable salt thereof, is about 5 mg. In some embodiments, a therapeutically effective amount of the compound of Formula I, II, III, IV, V, VI, or VII, or a pharmaceutically acceptable salt thereof, is about 10 mg. In some embodiments, a therapeutically effective amount of the compound of Formula I, II, III, IV, V, VI, or VII, or a pharmaceutically acceptable salt thereof, is about 15 mg. In some embodiments, a therapeutically effective amount of the compound of Formula I, II, III, IV, V, VI, or VII, or a pharmaceutically acceptable salt thereof, is about 20 mg. In some embodiments, a therapeutically effective amount of the compound of Formula I, II, III, IV, V, VI, or VII, or a pharmaceutically acceptable salt thereof, is about 25 mg. In some embodiments, a therapeutically effective amount of the compound of Formula I, II, III, IV, V, VI, or VII, or a pharmaceutically acceptable salt thereof, is about 30 mg. In some embodiments, a therapeutically effective amount of the compound of Formula I, II, III, IV, V, VI, or VII, or a pharmaceutically acceptable salt thereof, is about 35 mg. In some embodiments, a therapeutically effective amount of the compound of Formula I, II, III, IV, V, VI, or VII, or a pharmaceutically acceptable salt thereof, is about 40 mg. In some embodiments, a therapeutically effective amount of the compound of Formula I, II, III, IV, V, VI, or VII, or a pharmaceutically acceptable salt thereof, is about 45 mg. In some embodiments, a therapeutically effective amount of the compound of Formula I, II, III, IV, V, VI, or VII, or a pharmaceutically acceptable salt thereof, is about 50 mg.

In some embodiments, the methods described herein comprise administering to the subject an initial daily dose of about 1 to 500 mg of a compound provided herein, or a pharmaceutically acceptable salt thereof, and increasing the dose by increments until clinical efficacy is achieved. Increments of about 5, 10, 25, 50, or 100 mg can be used to increase the dose. The dosage can be increased daily, every other day, twice per week, once per week, once every two weeks, once every three weeks, or once a month.

When administered orally, the total daily dosage for a human subject may be between about 1 mg and 1,000 mg, between about 10-500 mg/day, between about 50-300 mg/day, between about 75-200 mg/day, or between about 100-150 mg/day. In some embodiments, the total daily dosage for a human subject may be about 5, 10, 25, 50, 75, 100, 200, 300, 400, 500, 600, 700, 800, 900, or 1000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose.

In some embodiments, the total daily dosage for a human subject may be about 10 mg/day administered in a single dose. In some embodiments, the total daily dosage for a human subject may be about 50 mg/day administered in a single dose. In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound provided herein, or a pharmaceutically acceptable salt thereof, is administered once daily in a method disclosed herein. In some embodiments, a compound provided herein, or a pharmaceutically acceptable salt thereof, is administered twice daily in a method disclosed herein.

The frequency of dosage of the compound of the present disclosure, or a pharmaceutically acceptable salt thereof, will be determined by the needs of the individual patient and can be, for example, once per day or twice, or more times, per day. Administration of the compound, or a pharmaceutically acceptable salt thereof, continues for as long as necessary to treat the inflammatory condition, or any other indication described herein. For example, a compound, or a pharmaceutically acceptable salt thereof, can be administered to a human suffering from an inflammatory condition for a period of from 20 days to 180 days or, for example, for a period of from 20 days to 90 days or, for example, for a period of from 30 days to 60 days.

Administration can be intermittent, with a period of several or more days during which a patient receives a daily dose of the compound of the present disclosure, or a pharmaceutically acceptable salt thereof, followed by a period of several or more days during which a patient does not receive a daily dose of the compound or a pharmaceutically acceptable salt thereof. For example, a patient can receive a dose of the compound, or a pharmaceutically acceptable salt thereof, every other day, or three times per week.

Again by way of example, a patient can receive a dose of the compound, or a pharmaceutically acceptable salt thereof, each day for a period of from 1 to 14 days, followed by a period of 7 to 21 days during which the patient does not receive a dose of the compound, or a pharmaceutically acceptable salt thereof, followed by a subsequent period (e.g., from 1 to 14 days) during which the patient again receives a daily dose of the compound, or a pharmaceutically acceptable salt thereof. Alternating periods of administration of the compound, or a pharmaceutically acceptable salt thereof, followed by non-administration of the compound, or a pharmaceutically acceptable salt thereof, can be repeated as clinically required to treat the patient.

The compounds of the present disclosure, or pharmaceutically acceptable salts thereof, or the pharmaceutical compositions of the present disclosure may be administered once, twice, three, or four times daily, using any suitable mode described above. Also, administration or treatment with the compounds, or pharmaceutically acceptable salts thereof, may be continued for a number of days; for example, commonly treatment would continue for at least 7 days, 14 days, or 28 days, for one cycle of treatment. Treatment cycles are well known for inflammatory conditions and other indications described herein. In some embodiments, treatment cycles are frequently alternated with resting periods of about 1 to 28 days, commonly about 7 days or about 14 days, between cycles. The treatment cycles, in other embodiments, may also be continuous.

### VI. Combination Therapy

Patients being treated by administration of the compounds provided herein, or pharmaceutically acceptable salts thereof, often exhibit diseases or conditions that benefit from treatment with other therapeutic agents. These diseases or conditions can be of an inflammatory nature or can be related to cancer, metabolic disorders, gastrointestinal disorders and the like. Thus, one embodiment of the disclosure is a method of treating an inflammation related disease or condition, or a metabolic disorder, gastrointestinal disorder, or cancer and the like comprising administering a compound of the present disclosure, or a pharmaceutically acceptable salt thereof, in combination with one or more compounds useful for the treatment of such diseases to a subject, particularly a human subject, in need thereof.

In some embodiments, a compound of the present disclosure, or a pharmaceutically acceptable salt thereof, is combined with one, two, three, four or more additional therapeutic agents. In some embodiments, a compound of the present disclosure, or a pharmaceutically acceptable salt thereof, is combined with two additional therapeutic agents. In some embodiments, a compound of the present disclosure, or a pharmaceutically acceptable salt thereof, is combined with three additional therapeutic agents. In some embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In some embodiments, when a compound of the present disclosure, or a pharmaceutically acceptable salt thereof, is combined with one or more additional therapeutic agents as described herein, the components of the composition are administered as a simultaneous or sequential regimen. When administered sequentially, the combination may be administered in two or more administrations.

In some embodiments, a compound of the present disclosure, or a pharmaceutically acceptable salt thereof, is combined with one or more additional therapeutic agents in a unitary dosage form for simultaneous administration to a patient, for example as a solid dosage form for oral administration.

In some embodiments, a compound of the present disclosure, or a pharmaceutically acceptable salt thereof, is co-administered with one or more additional therapeutic agents.

Co-administration includes administration of unit dosages of the compounds provided herein, or pharmaceutically acceptable salts thereof, before or after administration of unit dosages of one or more additional therapeutic agents. The compounds provided herein, or pharmaceutically acceptable salts thereof, may be administered within seconds, minutes, or hours of the administration of one or more additional therapeutic agents. For example, in some embodiments, a unit dose of a compound provided herein, or a pharmaceutically acceptable salt thereof, is administered first, followed within seconds or minutes by administration of a unit dose of one or more additional therapeutic agents. Alternatively, in other embodiments, a unit dose of one or more additional therapeutic agents is administered first, followed by administration of a unit dose of a compound provided herein, or a pharmaceutically acceptable salt thereof, within seconds or minutes. In some embodiments, a unit dose of a compound provided herein, or a pharmaceutically acceptable salt thereof, is administered first, followed, after a period of hours (i.e., 1-12 hours), by administration of a unit dose of one or more additional therapeutic agents. In other embodiments, a unit dose of one or more additional therapeutic agents is administered first, followed, after a period of hours (i.e., 1-12 hours), by administration of a unit dose of a compound provided herein or a pharmaceutically acceptable salt thereof.

In some embodiments, a compound of Formula I, II, III, IV, V, VI, or VII, or a pharmaceutically acceptable salt thereof, is formulated as a tablet, which may optionally contain one or more other compounds useful for treating the disease being treated. In certain embodiments, the tablet can contain another active ingredient for treating an inflammatory condition or other indication described herein. In some embodiments, such tablets are suitable for once daily dosing.

Also provided herein are methods of treatment in which a compound of Formula I, II, III, IV, V, VI, or VII, or a tautomer or pharmaceutically acceptable salt thereof, is given to a patient in combination with one or more additional therapeutic agents or therapy. In some embodiments, the total daily dosage of a compound of Formula I, II, III, IV, V, VI, or VII, or a tautomer, or a pharmaceutically acceptable salt thereof, may be about 1 to about 500 mg/day administered in a single dose for a human subject.

In some embodiments, the additional therapeutic agent is selected from the group consisting of anti-inflammatory drugs, anti-atherosclerotic drugs, immunosuppressive drugs, immunomodulatory drugs, cytostatic drugs, anti-proliferative agents, angiogenesis inhibitors, kinase inhibitors, cytokine blockers, and inhibitors of cell adhesion molecules.

In some embodiments, the additional therapeutic agent is selected from the group consisting of NSAIDs, immunosuppressive drugs, immunomodulatory drugs, cytostatic drugs, antiproliferative agents, angiogenesis inhibitors, biological agents, steroids, vitamin D3 analogs, retinoids, other kinase inhibitors, cytokine blockers, corticosteroids, and inhibitors of cell adhesion molecules. In some embodiments, the additional therapeutic agent is selected from the group consisting of torcetrapib, aspirin, niacin, HMG CoA reductase inhibitors (e.g., atorvastatin, fluvastatin, lovastatin, pravastatin, rosuvastatin and simvastatin), colesevelam, cholestyramine, colestipol, gemfibrozil, probucol, and clofibrate.

In some embodiments, the additional therapeutic agent is selected from the group consisting of corticosteroids, nonsteroidal anti-inflammatory drugs (NSAID) (e.g. ibuprofen, naproxen, acetaminophen, aspirin, Fenoprofen (Nalfon), Flurbiprofen (Ansaid), Ketoprofen, Oxaprozin (Daypro), Diclofenac sodium (Voltaren), Diclofenac potassium (Cataflam), Etodolac (Lodine), Indomethacin (Indocin), Ketorolac (Toradol), Sulindac (Clinoril), Tolmetin (Tolectin), Meclofenamate (Meclomen), Mefenamic acid (Ponstel), Nabumetone (Relafen), Piroxicam (Feldene), cox-2 inhibitors (e.g., celecoxib (Celebrex))), immunosuppressants (e.g., methotrexate (Rheumatrex), leflunomide (Arava), azathioprine (Imuran), cyclosporine (Neoral, Sandimmune), tacrolimus and cyclophosphamide (Cytoxan), CD20 blockers (Rituximab), Tumor Necrosis Factor (TNF) blockers (e.g., etanercept (Enbrel), infliximab (Remicade) and adalimumab (Humira)), Abatacept (CTLA4-Ig) and interleukin-1 receptor antagonists (e.g. Anakinra (Kineret), interleukin 6 inhibitors (e.g., Actemra), interleukin 17 inhibitors (e.g., AIN457), Janus kinase inhibitors (e.g., Tasocitinib), syk inhibitors (e.g. R788), and chloroquine and its derivatives.

In some embodiments, the additional therapeutic agent is selected from the group consisting of an EGFR kinase inhibitor, MEK inhibitor, VEGFR inhibitor, anti-VEGFR2 antibody, KDR antibody, AKT inhibitor, PDK-1 inhibitor, PI3K inhibitor, c-kit/Kdr tyrosine kinase inhibitor, Bcr-Abl tyrosine kinase inhibitor, VEGFR2 inhibitor, PDGFR-beta inhibitor, KIT inhibitor, Flt3 tyrosine kinase inhibitor, PDGF receptor family inhibitor, Flt3 tyrosine kinase inhibitor, RET tyrosine kinase receptor family inhibitor, VEGF-3 receptor antagonist, Raf protein kinase family inhibitor, angiogenesis inhibitor, Erb2 inhibitor, mTOR inhibitor, IGF-1R antibody, NFkB inhibitor, proteosome inhibitor, chemotherapy agent, and glucose reduction agent.

### Inflammatory Condition or Disease Combination Therapy

In some embodiments, the compounds provided herein, or pharmaceutically acceptable salts thereof, may be combined with one or more additional therapeutic agents that treat or ameliorate an inflammatory condition. Non-limiting examples of an inflammatory condition include, without limitation, acne, acid-induced lung injury, Addison's disease, adrenal hyperplasia, adrenocortical insufficiency, adult-onset Still's disease, adult respiratory distress syndrome (ARDS), age-related macular degeneration, aging, alcoholic hepatitis, alcoholic liver disease, allergen-induced asthma, allergic bronchopulmonary, allergic conjunctivitis, allergic contact dermatitis, allergies, allergic encephalomyelitis, allergic neuritis, allograft rejection, alopecia, alopecia areata, Alzheimer's disease, amyloidosis, amyotrophic lateral sclerosis, angina pectoris, angioedema, angiofibroma, anhidrotic ectodermal dysplasia-ill, anti-glomerular basement membrane disease, antigen-antibody complex mediated diseases, ankylosing spondylitis, antiphospholipid syndrome, aphthous stomatitis, appendicitis, arthritis, ascites, aspergillosis, asthma, atherosclerosis, atherosclerotic plaques, atopic dermatitis, atrophic thyroiditis, autoimmune diseases, autoimmune hemolytic anemia (immune pancytopenia, paroxysmal nocturnal hemoglobinuria), autoimmune polyendocrinopathies, autoimmune thrombocytopenia (idiopathic thrombocytopenic purpura, immune-mediated thrombocytopenia), autoimmune hepatitis, autoimmune thyroid disorders, autoinflammatory diseases, back pain, Bacillus anthracis infection, Bechet's disease, bee sting-induced inflammation, Behçet's syndrome, Bell's palsy, berylliosis, Blau syndrome, bone pain, bronchiolitis, bullous pemphigoid (BP) asthma, burns, bursitis, cardiac hypertrophy, carpal tunnel syndrome, Castleman's disease, catabolic disorders, cataracts, Celiac disease, cerebral aneurysm, chemical irritant-induced inflammation, chorioretinitis, chronic atypical neutrophilic dermatosis with lipodystrophy and elevated temperature (CANDLE) syndrome, chronic heart failure, chronic lung disease of prematurity, chronic obstructive pulmonary disease (COPD), chronic pancreatitis, chronic prostatitis, chronic recurrent multifocal osteomyelitis, cicatricial alopecia, colitis, complex regional pain syndrome, complications of organ transplantation, conjunctivitis, connective tissue disease, contact dermatitis, corneal graft neovascularization, corneal ulcer, Crohn's disease, cryopyrin-associated periodic syndromes, cutaneous lupus erythematosus (CLE), cryptococcosis, cystic fibrosis, deficiency of the interleukin-1 receptor antagonist (DIRA), dermatitis, dermatitis endotoxemia, dermatomyositis, diabetic macular edema, diverticulitis, eczema, encephalitis, endometriosis, endotoxemia, eosinophilic pneumonias, epicondylitis, epidermolysis bullosa, erythema multiforme, erythroblastopenia, esophagitis, familial amyloidotic polyneuropathy, familial cold urticarial, familial Mediterranean fever, fetal growth retardation, fibromyalgia, fistulizing Crohn's disease, food allergies, giant cell arteritis, glaucoma, glioblastoma, glomerular disease, glomerular nephritis, glomerulonephritis, gluten-sensitive enteropathy, gout, gouty arthritis, graft-versus-host disease (GVHD), granulomatous hepatitis, Graves' disease, growth plate injuries, Guillain-Barre syndrome. gut diseases, hair loss, Hashimoto's thyroiditis, head injury, headache, hearing loss, heart disease, hemangioma, hemolytic anemia, hemophilic joints, Henoch-Scholein purpura, hepatitis, hereditary periodic fever syndrome, heritable disorders of connective tissue, herpes zoster and simplex, hidradenitis suppurativa (HS), hip replacement, Hodgkin's disease, Huntington's disease, hyaline membrane disease, hyperactive inflammatory response, hyperammonemia, hypercalcemia, hypercholesterolemia, hypereosinophilic syndrome (HES), hyperimmunoglobulinemia D with recurrent fever (HIDS), hypersensitivity pneumonitis, hypertropic bone formation, hypoplastic and other anemias, hypoplastic anemia, ichthyosis, idiopathic demyelinating polyneuropathy, Idiopathic inflammatory myopathies (dermatomyositis, polymyositis), idiopathic pulmonary fibrosis, idiopathic thrombocytopenic purpura, immunoglobulin nephropathies, immune complex nephritis, immune thrombocytopenic purpura (ITP), incontinentia pigmenti (IP, Bloch-Siemens syndrome), infectious mononucleosis, infectious diseases including viral diseases such as AIDS (HIV infection), hepatitis A, B, C, D, and E, herpes; inflammation, inflammation of the CNS, inflammatory bowel disease (IBD), inflammatory disease of the lower respiratory tract including bronchitis or chronic obstructive pulmonary diseases, inflammatory disease of the upper respiratory tract including the nose and sinuses such as rhinitis or sinusitis, inflammatory diseases of the respiratory tract, inflammatory ischemic event such as stroke or cardiac arrest, inflammatory lung disease, inflammatory myopathy such as myocarditis, inflammatory liver disease, inflammatory neuropathy, inflammatory pain, insect bite-induced inflammation, interstitial cystitis, interstitial lung disease, iritis, irritant-induced inflammation, ischemia/reperfusion, joint replacement, juvenile arthritis, juvenile rheumatoid arthritis, keratitis, kidney injury caused by parasitic infections, kidney transplant rejection, leptospirosis, leukocyte adhesion deficiency, lichen sclerosus (LS), Lambert-Eaton myasthenic syndrome, Loeffler's syndrome, lupus, lupus nephritis, Lyme disease, Marfan syndrome (MFS), mast cell activation syndrome, mastocytosis, meningitis, meningioma, mesothelioma, mixed connective tissue disease, Muckle-Wells syndrome (urticaria deafness amyloidosis), mucositis, multiple organ injury syndrome, multiple sclerosis, muscle wasting, muscular dystrophy, myasthenia gravis (MG), myelodysplastic syndrome, myocarditis, myositis, nasal sinusitis, necrotizing enterocolitis, neonatal onset multisystem inflammatory disease (NOMID), neovascular glaucoma, nephrotic syndrome, neuritis, neuropathological diseases, non-allergen induced asthma, obesity, ocular allergy, optic neuritis, organ transplant rejection, Osier-Weber syndrome, osteoarthritis, osteogenesis imperfecta, osteonecrosis, osteoporosis, osteoarthritis, otitis, pachyonychia congenita, Paget's disease, Paget's disease of bone, pancreatitis, Parkinson's disease, pediatric rheumatology, pelvic inflammatory disease, pemphigus, pemphigus vulgaris (PV), bullous pemphigoid (BP), pericarditis, periodic fever, periodontitis, peritoneal endometriosis, pernicious anemia (Addison's disease), pertussis, PFAPA (periodic feveraphthous pharyngitis and cervical adenopathy), pharyngitis and adenitis (PFAPA syndrome), plant irritant-induced inflammation, pneumocystis infection, pneumonia, pneumonitis, poison ivy/urushiol oil-induced inflammation, polyarthritis nodosa, polychondritis, polycystic kidney disease, polymyalgia rheumatic, giant cell arteritis, polymyositis, pouchitis, reperfusion injury and transplant rejection, primary biliary cirrhosis, primary pulmonary hypertension, primary sclerosing cholangitis (PSC), proctitis, psoriasis, psoriasis vulgaris, psoriatic arthritis, psoriatic epidermis, psychosocial stress diseases, pulmonary disease, pulmonary fibrosis, pulmonary hypertension, pyoderma gangrenosum, pyogenic granuloma retrolental fibroplasias, pyogenic sterile arthritis, Raynaud's syndrome, Reiter's disease, reactive arthritis, renal disease, renal graft rejection, reperfusion injury, respiratory distress syndrome, retinal disease, retrolental fibroplasia, Reynaud's syndrome, rheumatic carditis, rheumatic diseases, rheumatic fever, rheumatoid arthritis, rhinitis, rhinitis psoriasis, rosacea, sarcoidosis, Schnitzler syndrome, scleritis, sclerosis, scleroderma, scoliosis, seborrhea, sepsis, septic shock, severe pain, Sezary syndrome, sickle cell anemia, silica-induced disease (Silicosis), Sjogren's syndrome, skin diseases, skin irritation, skin rash, skin sensitization (contact dermatitis or allergic contact dermatitis), sleep apnea, spinal cord injury, spinal stenosis, spondyloarthropathies, sports injuries, sprains and strains, Stevens-Johnson syndrome (SJS), stroke, subarachnoid hemorrhage, sunburn, synovial inflammation, systemic inflammatory response syndrome (SIRS), systemic lupus erythematosus (SLE), systemic mast cell disease (SMCD), systemic vasculitis, systemic-onset juvenile idiopathic arthritis, temporal arteritis, tendinitis, tenosynovitis, thrombocytopenia, thyroditis, thyroiditis, tissue transplant, toxoplasmosis, trachoma, transplantation rejection, traumatic brain injury, tuberculosis, tubulointerstitial nephritis, tumor necrosis factor (TNF) receptor associated periodic syndrome (TRAPS), type 1 diabetes, type 2 diabetes, complications from type 1 or type 2 diabetes, ulcerative colitis, urticaria, uterine fibroids, uveitis, uveoretinitis, vascular restenosis, vasculitis, vasculitis (NHLBI), vitiligo, Wegener's granulomatosis, and Whipple's disease.

Non-limiting examples of therapeutic agents for treatment of an inflammatory disease or condition that can be used in combination with the compounds provided herein, or pharmaceutically acceptable salts thereof, include alpha-fetoprotein modulators; adenosine A3 receptor antagonist; adrenomedullin ligands; AKT1 gene inhibitors; antibiotics; antifungals; ASK1 inhibitors; ATPase inhibitors; beta adrenoceptor antagonists; BTK inhibitors; calcineurin inhibitors; carbohydrate metabolism modulators; cathepsin S inhibitors; CCR9 chemokine antagonists; CD233 modulators; CD29 modulators; CD3 antagonists; CD40 ligand inhibitors; CD40 ligand receptor antagonists; chemokine CXC ligand inhibitors; CHST15 gene inhibitors; collagen modulators; COT protein kinase inhibitors; CSF-1 agonist; CSF-1 antagonists; CX3CR1 chemokine modulators DYRK-1 alpha protein kinase inhibitor, eotaxin ligand inhibitors; EP4 prostanoid receptor agonists; F1F0 ATP synthase modulators; farnesoid X receptor (FXR, NR1H4) agonists or modulators; fecal microbiota transplantation (FMT), fractalkine ligand inhibitors; free fatty acid receptor 2 antagonists; GATA 3 transcription factor inhibitors; glucagon-like peptide 2 agonists; glucocorticoid agonists; Glucocorticoid receptor modulators; guanylate cyclase receptor agonists; HIF prolyl hydroxylase inhibitors; histone deacetylase inhibitors; HLA class II antigen modulators; hypoxia inducible factor-1 stimulator; ICAM1 gene inhibitors; IL-1 beta ligand modulators; IL-12 antagonists; IL-13 antagonists; IL-18 antagonists; IL-18 receptor accessory protein antagonist, IL-22 agonists; IL-23 antagonists; IL-23A inhibitors; IL-6 antagonists; IL-7 receptor antagonists; IL-8 receptor antagonists; IL-36 inhibitors, integrin alpha-4/beta-1 antagonists; integrin alpha-4/beta-7 antagonists; integrin antagonists; interleukin ligand inhibitors; interleukin receptor 17A antagonists; interleukin-1 beta ligands; interleukin 1 like receptor2 inhibitors; IL-6 receptor modulators; JAK tyrosine kinase inhibitors; Jak1 tyrosine kinase inhibitors; Jak3 tyrosine kinase inhibitors; lactoferrin stimulators; LanC like protein 2 modulators; leukocyte elastase inhibitors; leukocyte proteinase-3 inhibitors; MAdCAM inhibitors; melanin concentrating hormone (MCH-1) antagonist; melanocortin agonists; metalloprotease-9 inhibitors; microbiome-targeting therapeutics; natriuretic peptide receptor C agonists; neuregulin-4 ligands; NLRP3 inhibitors; NKG2 D activating NK receptor antagonists; NR1H4 receptor (FXR) agonists or modulators (deleted); nuclear factor kappa B inhibitors; opioid receptor antagonists; OX40 ligand inhibitors; oxidoreductase inhibitors; P2X7 purinoceptor modulators; PDE 4 inhibitors; Pellino homolog 1 inhibitors; PPAR alpha/delta agonists; PPAR gamma agonists; Protein arginine deiminase IV inhibitor, protein fimH inhibitors; P-selectin glycoprotein ligand-1 inhibitors; Ret tyrosine kinase receptor inhibitors; RIP-1 kinase inhibitors; RIP-2 kinase inhibitors; RNA polymerase inhibitors; sphingosine 1 phosphate phosphatase 1 stimulators; sphingosine-1-phosphate receptor-1 agonists; sphingosine-1-phosphate receptor-5 agonists; sphingosine-1-phosphate receptor-1 antagonists; sphingosine-1-phosphate receptor-1 modulators; stem cell antigen-1 inhibitors; superoxide dismutase modulators; SYK inhibitors; tissue transglutaminase inhibitor; TLR-3 antagonists; TLR-4 antagonists; Toll-like receptor 8 (TLR8) inhibitors; TNF alpha ligand inhibitors; TNF ligand inhibitors; TNF alpha ligand modulators; TNF antagonists; TPL-2 inhibitors; tumor necrosis factor 14 ligand modulators; tumor necrosis factor 15 ligand inhibitors; Tyk2 tyrosine kinase inhibitors; type I IL-1 receptor antagonists; vanilloid VR1 agonists; and zonulin inhibitors; or any combination thereof.

Adenosine A3 receptor antagonists include but are not limited to PBF-677.

Adrenomedullin ligands include but are not limited to adrenomedullin.

Antibiotics include but are not limited to ciprofloxacin, clarithromycin, metronidazole, vancomycin, rifamycin, rifaximin, and tosufloxacin.

ASK1 inhibitors include but are not limited to GS-4997.

Alpha-fetoprotein modulators include but are not limited to ACT-101.

Anti-CD28 inhibitors include but are not limited to JNJ-3133 and abatacept.

Beta adrenoceptor antagonists include but are not limited to NM-001.

BTK inhibitors include but are not limited to GS-4059.

Calcineurin inhibitors include but are not limited to tacrolimus and ciclosporin.

Carbohydrate metabolism modulators include but are not limited to ASD-003.

Cathepsin S inhibitors include but are not limited to VBY-129.

CCR9 chemokine antagonists include but are not limited to CCX-507.

CD233 modulators include but are not limited to GSK-2831781.

CD29 modulators include but are not limited to PF-06687234.

CD3 antagonists include but are not limited to NI-0401, muromonab-CD3, and teplizumab.

CD4 antagonists include but are not limited to IT-1208.

CD40 ligand inhibitors include but are not limited to SAR-441344 and letolizumab.

CD40 gene inhibitors include but are not limited to NJA-730.

CD40 ligand receptor antagonists include but are not limited to FFP-104, BI-655064, ABBV-323, and VIB-4920.

Chaperonin binding immunoglobulin protein include but are not limited to IRL-201805.

Chemokine CXC ligand inhibitors include but are not limited to LY-3041658.

CHST15 gene inhibitors include but are not limited to STNM-01.

Collagen modulators include but are not limited to ECCS-50 (DCCT-10).

COT protein kinase inhibitors include but are not limited to GS-4875.

CSF-1 antagonists include but are not limited to JNJ-40346527 (PRV-6527) and SNDX-6352.

CX3CR1 chemokine modulators include but are not limited to E-6130.

DYRK-1 alpha protein kinase inhibitor include but are not limited to VRN-02.

Microbiome-targeting therapeutics include but are not limited to SER-287, SER-301, and SER-155.

Eotaxin ligand inhibitors include but are not limited to bertilimumab.

EP4 prostanoid receptor agonists include but are not limited to KAG-308.

F1F0 ATP synthase modulators include but are not limited to LYC-30937 EC.

Fractalkine ligand inhibitors include but are not limited to quetmolimab (E-6011).

Free fatty acid receptor 2 antagonists include but are not limited to GLPG-0974.

GATA 3 transcription factor inhibitors include but are not limited to SB-012.

Glucagon-like peptide 2 agonists include but are not limited to teduglutide and apraglutide.

Glucocorticoid receptor agonists include but are not limited to budesonide, beclomethasone dipropionate, and dexamethasone sodium phosphate.

Glucocorticoid receptor modulators/TNF ligand inhibitors include but are not limited to ABBV-3373.

Guanylate cyclase receptor agonists include but are not limited to dolcanatide.

HIF prolyl hydroxylase inhibitors include but are not limited to DS-1093 and AKB-4924.

HIF prolyl hydroxylase-2 inhibitors/hypoxia inducible factor-1 stimulators include but are not limited to GB-004.

Histone deacetylase inhibitors include but are not limited to givinostat and NIPEP-CARE.

Histone deacetylase-6 inhibitors include but are not limited to CKD-506.

HLA class II antigen modulators include but are not limited to HLA class II protein modulators.

ICAM1 gene inhibitors include but are not limited to alicaforsen.

IL-12 antagonists include but are not limited to ustekinumab (IL12/IL23).

IL-13 antagonists include but are not limited to tralokinumab.

IL-18 antagonists include but are not limited to GSK-1070806.

IL-18 receptor accessory protein antagonist include but are not limited to anti-IL-1R7 canonical antibody.

IL-22 agonists include but are not limited to AMT-126 and RG-7880.

IL-23 antagonists include but are not limited to tildrakizumab, risankizumab (BI-655066), mirikizumab (LY-3074828), brazikumab (AMG-139), IBI-112, and PTG-200.

IL-23A inhibitors include but are not limited to guselkumab.

IL-6 antagonists include but are not limited to olokizumab.

IL-7 receptor antagonists include but are not limited to OSE-127.

IL-8 receptor antagonists include but are not limited to clotrimazole.

Integrin alpha-4/beta-1 antagonists include but are not limited to natalizumab.

Integrin alpha-4/beta-7 antagonists include but are not limited to etrolizumab (a4b7/aEb7), vedolizumab, carotegrast methyl, TRK-170 (a4b7/a4b1), PTG-100, and PN-10943.

Integrin antagonists include but are not limited to E-6007.

Interleukin ligand inhibitors include but are not limited to bimekizumab (IL-17A/IL-17F).

Interleukin receptor 17A antagonists include but are not limited to brodalumab.

Interleukin-1 beta ligands include but are not limited to K(D)PT.

Interleukin 1 like receptor 2 inhibitors include but are not limited to BI-655130.

IL-6 receptor modulators include but are not limited to Amilo-5MER and olamkicept.

JAK tyrosine kinase inhibitors include but are not limited to tofacitinib (1/3), peficitinib (1/3), TD-3504, and TD-1473.

Jak1 tyrosine kinase inhibitors include but are not limited to a compound disclosed in U.S. Pat. No. 9,238,628.

Jak3 tyrosine kinase inhibitors include but are not limited to OST-122 and PF-06651600.

Jak3 tyrosine kinase inhibitor/TrkA receptor antagonist include but are not limited to SNA-125.

Examples of other JAK inhibitors include but are not limited to AT9283, AZD1480, baricitinib, BMS-911543, fedratinib, filgotinib (GLPG0634), gandotinib (LY2784544), INCB039110, lestaurtinib, momelotinib (CYT0387), NS-018, pacritinib (SB1518), peficitinib (ASP015K), ruxolitinib, tofacitinib (formerly tasocitinib), XL019, upadacitinib (ABT-494), LPG-0555, SHR-0302, and brepocitinib (PF-06700841) (JAK1/Tyk2).

Lactoferrin stimulators include but are not limited to recombinant human lactoferrin (VEN-100).

LanC like protein 2 modulators include but are not limited to BT-11 and BT-104.

Leukocyte elastase inhibitors/Leukocyte proteinase-3 inhibitors include but are not limited to tiprelestat.

MAdCAM inhibitors include but are not limited to SHP-647 (PF-547659).

Melanin concentrating hormone (MCH-1) antagonists include but are not limited to CSTI-100.

Melanocortin MC1 receptor agonists include but are not limited to ASP-3291 and PL-8177.

Metalloprotease-9 inhibitors include but are not limited to GS-5745.

Microbiome modulators include but are not limited to ABI-M201.

Natriuretic peptide receptor C agonists include but are not limited to plecanatide.

Neuregulin-4 ligands include but are not limited to NRG-4.

NKG2 D activating NK receptor antagonists include but are not limited to JNJ-4500.

NLRP3 inhibitors include but are not limited to dapansutrile, BMS-986299, SB-414, MCC-950, IFM-514, JT-194, PELA-167, and NBC-6.

Farnesoid X receptor (FXR, NR1H4) agonists or modulators include but are not limited to AGN-242266, cilofexor tromethamine (GS-9674), EDP-305, EYP-001, GNF-5120, MET-409, MET-642, nidufexor (LMB-763), obeticholic acid, TERN-101, and tropifexor.

Nuclear factor kappa B inhibitors include but are not limited to Thetanix.

Opioid receptor antagonists include but are not limited to naltrexone and IRT-103.

OX40 ligand inhibitors include but are not limited to KHK-4083.

Oxidoreductase inhibitors include but are not limited to olsalazine.

Pellino homolog 1 inhibitors include but are not limited to BBT-401.

P2X7 purinoceptor modulators include but are not limited to SGM-1019.

PDE 4 inhibitors include but are not limited to apremilast.

PPAR alpha/delta agonists include but are not limited to elafibranor (GFT-1007).

PPAR gamma agonists include but are not limited to GED-0507-34-Levo.

Protein fimH inhibitors include but are not limited to sibofimloc (EB-8018).

P-selectin glycoprotein ligand-1 inhibitors include but are not limited to SEL-K2, AbGn-168H, and neihulizumab.

Ret tyrosine kinase receptor inhibitors include but are not limited to GSK-3179106.

RIP-1 kinase inhibitors include but are not limited to GSK-2982772 and VRN-04.

RIP-2 kinase inhibitors include but are not limited to GSK-2983559.

Sphingosine 1 phosphate phosphatase 1 stimulators include but are not limited to etrasimod.

Sphingosine-1-phosphate receptor-1 agonists include but are not limited to mocravimod (KRP-203) and BMS-986166.

Sphingosine-1-phosphate receptor-1 agonists/Sphingosine-1-phosphate receptor-5 agonists include but are not limited to ozanimod.

Sphingosine-1-phosphate receptor-1 antagonists include but are not limited to amiselimod (MT-1303).

Sphingosine-1-phosphate receptor-1 modulators include but are not limited to OPL-002, SK1-I.

Stem cell antigen-1 inhibitors include but are not limited to Ampion (DMI-9523).

Superoxide dismutase modulators include but are not limited to midismase.

Syk inhibitors include but are not limited to GS-9876.

tissue transglutaminase inhibitor include but are not limited to zampilimab.

TLR-3 antagonists include but are not limited to PRV-300.

TLR-4 antagonists include but are not limited to JKB-122.

Toll-like receptor 8 (TLR8) inhibitors include but are not limited to E-6887, IMO-4200, IMO-8400, IMO-9200, MCT-465, MEDI-9197, motolimod, resiquimod, VTX-1463, and VTX-763.

TNF alpha ligand inhibitors include but are not limited to adalimumab, certolizumab pegol, infliximab, golimumab, DLX-105, Debio-0512, HMPL-004, CYT-020-TNFQb, Hemay-007, and V-565.

TNF alpha ligand modulators/IL-1 betaligand modulators include but are not limited to PUR-0110.

TNF antagonists include but are not limited to AVX-470, tulinercept, and etanercept.

Tumor necrosis factor 14 ligand modulators include but are not limited to AEVI-002.

Tumor necrosis factor 15 ligand inhibitors include but are not limited to PF-06480605.

Tyk2 tyrosine kinase inhibitors include but are not limited to PF-06826647 and BMS-986165.

Type I IL-1 receptor antagonists include but are not limited to anakinra.

Zonulin inhibitors include but are not limited to larazotide acetate.

In some embodiments, the compounds provided herein, or pharmaceutically acceptable salts thereof, may be combined with one or more anti-inflammatory agents. Anti-inflammatory agents include but are not limited to non-steroidal anti-inflammatory drugs (NSAIDs), non-specific and COX-2 specific cyclooxygenase enzyme inhibitors, gold compounds, corticosteroids, methotrexate, tumor necrosis factor receptor (TNF) receptors antagonists, and immunosuppressants.

Examples of NSAIDs include, but are not limited to ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Additional examples of NSAIDs also include but are not limited to COX-2 specific inhibitors (i.e., a compound that inhibits COX-2 with an IC₅₀ that is at least 50-fold lower than the IC₅₀ for COX-1), such as celecoxib, valdecoxib, lumiracoxib, etoricoxib and/or rofecoxib.

In some embodiments, the anti-inflammatory agent is a salicylate. Salicylates include but are not limited to acetylsalicylic acid or aspirin, sodium salicylate, choline, and magnesium salicylates.

In some embodiments, the anti-inflammatory agent is a corticosteroid. Non-limiting examples of a corticosteroid include cortisone, dexamethasone, methylprednisolone, prednisolone, prednisolone sodium phosphate, and prednisone.

In some embodiments, the anti-inflammatory agent is a gold compound, e.g., gold sodium thiomalate or auranofin.

In some embodiments, the anti-inflammatory agent is a metabolic inhibitor. Non-limiting examples of a metabolic inhibitor include a dihydrofolate reductase inhibitor, such as methotrexate, or a dihydroorotate dehydrogenase (DHODH) inhibitor, such as leflunomide.

In some embodiments, the anti-inflammatory agent is an anti-C5 monoclonal antibody (such as eculizumab or pexelizumab), a TNF antagonist (such as etanercept), or infliximab, which is an anti-TNF alpha monoclonal antibody.

In some embodiments, the anti-inflammatory agent is an immunosuppressant. Non-limiting examples of an immunosuppressant include methotrexate, leflunomide, cyclosporine, tacrolimus, azathioprine, mycophenolate sodium, mercaptopurine, and mycophenolate mofetil.

### Lupus Combination Therapy

In some embodiments, the compounds provided herein, or pharmaceutically acceptable salts thereof, is combined with one or more additional therapeutic agents that target adenosylhomocysteinase, ADP ribosyl cyclase-1 (CD38), adrenocorticotrophic hormone ligands, AIMP multisynthetase complex protein 1, annexin A1 modulators, B and T lymphocyte attenuator (BTLA), BDCA2, beta 2 adrenoceptor, B-lymphocyte antigen CD19, B-lymphocyte antigen CD20, B-lymphocyte cell adhesion molecule (CD22), B-lymphocyte stimulator ligand (BAFF), btk tyrosine kinase, cannabinoid CB2 receptor, CD11b agonists, CD38 Activation-inducible TNF receptor, CD40 (CD154) ligand, CD74, CD79b modulators, CDw123, Collagen VII (Col VII), Complement C5 factor, C-type lectin domain protein 4C, CXCR5 chemokine modulators, deoxyribonuclease modulators, DNA binding protein Ikaros, DYRK-1 alpha protein kinase, dndoplasmin, Exportin 1, FK506 binding protein, glucocorticoid receptor, HLA antigen, IL-10, IL-23m IL-12 receptors, IL-2 receptor, IL-2 receptor alpha subunit, IL-21 modulators, IL-6R, immunoglobulin gamma Fc receptor II modulators, immunoglobulin gamma Fc receptor IIB, inducible T-cell co-stimulator, interferon alpha ligand (INF-alpha), interferon omega ligand (INF omega), interferon type I receptor, interleukin-2 ligand, Itk tyrosine kinase, JAK tyrosine kinase, Jak1 tyrosine kinase, Jak2 tyrosine kinase, Jak3 tyrosine kinase, KCNA voltage-gated potassium channel-3, leukocyte Ig like receptor A4 modulators, mitochondrial 10 kDa heat shock protein, mTOR, non receptor tyrosine kinase TYK2, nuclear export, nuclear factor kappa B inducing kinase, nuclease stimulators, OX-40 receptors, PARP modulators, proteasome modulators, protein arginine deiminase IV (PAD4), protein cereblon modulators, protein MB21D1, retinoid Z receptor gamma inverse, rho associated protein kinase 1, rho associated protein kinase 2, serine threonine protein kinase TBK1 (TBK1), sphingosine kinase 1, sphingosine-1-phosphate receptor-1 modulators, stimulator of interferon genes protein, Syk tyrosine kinase, T cell surface glycoprotein CD28, T-cell differentiation antigen CD6, TLR-7 modulators, TLR-8 modulators, TLR-9 modulators, transcription factor modulators, tumor necrosis factor ligand 13 (APRIL), Tyk2 tyrosine kinase, ubiquitin ligase modulators, and/or zinc finger binding protein Aiolos.

In some embodiments, the compounds provided herein, or pharmaceutically acceptable salts thereof, may be combined with one, two, three, or four additional therapeutic agents selected from:
activation-inducible TNF receptor agonists, including but not limited to BMS-986256;
adenosylhomocysteinase inhibitors, including but not limited to DZ-2002;
adrenocorticotrophic hormone ligands, including but not limited to corticotropin;
AIMP multisynthetase complex protein 1 stimulator/Endoplasmin inhibitors, including but not limited to anchorins;
anti-CDw123 antibodies, including but not limited to talacotuzumab;
annexin A1 modulators, including but not limited to annexuzlimab;
anti-IL-12/IL23 antibodies, including but not limited to AK-101;
anti-BAFF-R antibodies, including but not limited to lanalumab;
anti-BDCA2 antibodies, including but not limited to BIIB-059;
anti-BLys antibodies, including but not limited to belimumab and UBP-1213;
anti-BTLA modulator antibodies, including but not limited to LY-3361237;
anti-C5 antibodies, including but not limited to eculizumab;
anti-CD154 antibodies, including but not limited to TNX-1500;
anti-CD19/CD32b antibodies, including but not limited to obexelimab;
anti-CD20 antibodies, including but not limited to veltuzumab;
anti-CD22 antibodies, including but not limited to SM-06, SM-03;
anti-CD28 antibodies, including but not limited to theralizumab;
anti-CD38 antibodies, including but not limited to TAK-079 and felzartamab;
anti-CD40 antibodies, including but not limited to iscalimab and dapirolizumab pegol;
anti-CD6 antibodies, including but not limited to itolizumab;
anti-CD74 antibodies, including but not limited to milatuzumab;
anti-CXCR5 antibodies, including but not limited to PF-06835375;
anti-IFN-alpha antibodies, including but not limited to QX-006-N;
anti-IFN-alpha/omega antibodies, including but not limited to JNJ-55920839;
anti-IL-10 antibodies, including but not limited to BT-063;
anti-IL-21 antibodies, including but not limited to BOS-161721;
anti-IL-6R nanobodies, including but not limited to vobarilizumab;
anti-ILT7 antibodies, including but not limited to daxdilimab;
anti-interferon alpha vaccines, including but not limited to CKD-971;
anti-interferon receptor type I antibodies, including but not limited to anifrolumab;
anti-PAD4 antibodies, including but not limited to PFI-102;
anti-TLR-7 antibodies, including but not limited to DS-7011;
BAFF/APRIL inhibitors, including but not limited to ALPN-303;
Beta 2 adrenoceptor agonists, including but not limited to R-salbutamol sulphate;
bi-specific antibodies targeting BAFF/ICOSL, including but not limited to rozibafusp alfa;
bi-specific antibodies targeting CD32B/CD79B, including but not limited to PRV-3279;
bi-specific antibodies targeting Col VII/BAFF, including but not limited to TE-2324;
B-lymphocyte stimulator ligand inhibitors, including but not limited to atacicept and telitacicept;
Btk tyrosine kinase inhibitors, including but not limited to AC-0058, fenebrutinib, XNW-1011, tirabrutinib hydrochloride, branebrutinib, elsubrutinib, and orelabrutinib;
Btk/itk tyrosine kinase inhibitors, including but not limited to DWP-213388;
Btk/Jak3 tyrosine kinase inhibitors, including but not limited to DWP-212525;
cannabinoid CB2 receptor agonists, including but not limited to julemic acid;
CD11b agonists, including but not limited to LA-1;
deoxyribonuclease gamma stimulators, including but not limited to NTR-441;
deoxyribonuclease modulators, including but not limited to Oshadi D;
DYRK-1 alpha protein kinase inhibitors, including but not limited to VRN-02;
exportin 1 inhibitors, including but not limited to SINE compounds;
glucocorticoid receptor agonists, including but not limited to prednisone;
HLA antigen modulators, including but not limited to PEGylated HLA-x (SLE);
IL-2 receptor alpha subunit stimulators, including but not limited to NKTR-35;
immunoglobulin gamma Fc receptor IIB modulators, including but not limited to valziflocept;
inducible T-cell co-stimulator inhibitor (ICOS)/T cell surface glycoprotein CD28 inhibitors, including but not limited to ALPN-101;
interferon alpha ligand modulator/TLR-7/TLR-9 modulators, including but not limited to DV-1079;
interleukin-2 ligands, including but not limited to interleukin-2 Anteluke, interking recombinant human interleukin-2, ILT-101, and CUG-252;
interleukin-2 ligands/IL-2 receptor agonists, including but not limited to interleukin-2 follow-on biologic;
JAK 1/2/3 and ROCK 1/2 inhibitors, including but not limited to CPL-409116;
JAK tyrosine kinase inhibitors, including but not limited to delgocitinib;
Jak1/Jak2 tyrosine kinase inhibitors, including but not limited to baricitinib;
Jak1 tyrosine kinase inhibitors, including but not limited to upadacitinib, filgotinib, itacitinib, and INCB-54707;
Jak1/Tyk2 tyrosine kinase inhibitors, including but not limited to brepocitinib, SDC-1801, and SDC-1802;
JAK3/1 and TBK1 kinase inhibitors, including but not limited to CS-12192;
JAK3/JAK1 tyrosine kinase inhibitors, including but not limited to tofacitinib citrate;
KCNA voltage-gated potassium channel-3 inhibitors, including but not limited to dalazatide;
mitochondrial 10 kDa heat shock protein stimulators, including but not limited to INV-103;
mTOR inhibitors, including but not limited to TAM-01;
non-receptor tyrosine kinase TYK2 antagonists, including but not limited to ICP-330;
nuclear export inhibitors, including but not limited to verdinexor;
nuclear factor kappa B inducing kinase inhibitors, including but not limited to NIK-SMI1;
nuclease stimulators, including but not limited to RSLV-132;
OX-40 receptor antagonists, including but not limited to ISB-830;
PARP modulators, including but not limited to bendamustine hydrochloride;
PD-L1 CAR-expressing NK-92 cell therapy;
proteasome inhibitors, including but not limited to KZR-616;
protein cereblon modulators, including but not limited to iberdomide;
protein MB21D1 inhibitors, including but not limited to X-6;
retinoid Z receptor gamma inverse agonists, including but not limited to INV-17;
sphingosine kinase 1 inhibitors, including but not limited to BML-258;
sphingosine-1-phosphate receptor-1 modulator, including but not limited to cenerimod;
Syk tyrosine kinase inhibitors, including but not limited to GSK-2646264, SKI-O-703, lanraplenib (GS-9876), GNS-1653, and HMPL-523;
TLR-9 antagonists, including but not limited to chloroquine, hydroxychloroquine, hydroxychloroquine sulfate, COV-08-0064; GNKS-356, and AVO-101;
TLR7/8 antagonists, including but not limited to M-5049, E-6887, and BMS-986256;
TLR-8 antagonists, including but not limited to ZG-170607;
TLR7/8/9 antagonists, including but not limited to IMO-8400 and IMO-9200;
Tyk2 tyrosine kinase inhibitors, including but not limited to deucravacitinib;
ubiquitin ligase modulators, including but not limited to KPG-818; and
other drugs for lupus, including but not limited to mometasone, betamethasone, forigerimod, anandamide, DCB-SLE1, arsenic trioxide, tairuimide, TV-4710 (edratide), allogeneic human umbilical cord-derived mesenchymal stem cell therapy (hUC-MSCs), LC-200, BI-705564, SM-934, GX-101, TXR-712, TXR-711, CIT-013, MHV-370, Panzyga®, TPX-6001, TPX-7001, artenimol, AMG-592, phosphatidylserine-liposome-based immunotherapy, and CD4+CD127lo/−CD25+ polyclonal regulatory T cells.

In some embodiments, the compounds provided herein, or pharmaceutically acceptable salts thereof, may be combined with one, two, three, or four additional therapeutic agents selected from veltuzumab, PF-06835375, eculizumab, milatuzumab, SM-06, SM-03, BT-063, QX-006-N, BOS-161721, AK-101, TNX-1500, theralizumab, daxdilimab, TAK-079, felzartamab, itolizumab, anifrolumab, iscalimab, dapirolizumab pegol, lanalumab, LY-3361237, JNJ-55920839, UBP-1213, DS-7011, PFI-102, BIIB-059, obexelimab, talacotuzumab, vobarilizumab, TE-2324, PRV-3279, chloroquine, hydroxychloroquine, hydroxychloroquine sulfate, COV-08-0064; GNKS-356, AVO-101, rozibafusp alfa, VRN-02, annexuzlimab, ALPN-101, bendamustine hydrochloride, BMS-986256, NKTR-35, atacicept, telitacicept, BMS-986256, M-5049, KZR-616, KPG-818, verdinexor, ALPN-303, valziflocept, LA-1, cenerimod, prednisone, corticotropin, deucravacitinib, CPL-409116, CS-12192, tofacitinib citrate, ISB-830, DV-1079, julemic acid, iberdomide, TAM-01, BML-258, brepocitinib, SDC-1801, SDC-1802, ICP-330, NTR-441, dalazatide, GSK-2646264, SKI-O-703, lanraplenib (GS-9876), GNS-1653, HMPL-523, RSLV-132, interleukin-2 follow-on biologic, interleukin-2 Anteluke, interking recombinant human interleukin-2, ILT-101, CUG-252, DZ-2002, PEGylated HLA-x (SLE), AC-0058, fenebrutinib, XNW-1011, tirabrutinib hydrochloride, branebrutinib, elsubrutinib, orelabrutinib, DWP-213388, INV-103, R-salbutamol sulphate, anchorins, NIK-SMI1, X-6, INV-17, Oshadi D, baricitinib, upadacitinib, filgotinib, itacitinib, INCB-54707, delgocitinib, DWP-212525, CKD-971, as mometasone, betamethasone, forigerimod, anandamide, DCB-SLE1, arsenic trioxide, tairuimide, TV-4710 (edratide), allogeneic human umbilical cord-derived mesenchymal stem cell therapy (hUC-MSCs), LC-200, BI-705564, SM-934, GX-101, TXR-712, TXR-711, CIT-013, MHV-370, Panzyga®, TPX-6001, TPX-7001, artenimol, and AMG-592, or a pharmaceutically acceptable salt of any of the foregoing, or any combination thereof.

### Psoriasis Combination Therapy

In some embodiments, the compounds provided herein, or pharmaceutically acceptable salts thereof, may be combined with one, two, three, or four additional therapeutic agents that are useful for treating or ameliorating psoriasis. In some embodiments, the compounds provided herein, or pharmaceutically acceptable salts thereof, may be combined with one, two, three, or four additional therapeutic agents selected from acetaldehyde dehydrogenase inhibitor, adenosine A1 receptor antagonist, adenosine A3 receptor antagonist, adenosine A3 receptor agonists, ADP ribosyl cyclase-1 inhibitors, alpha 2 adrenoceptor modulator, apolipoprotein A antagonist, aryl hydrocarbon receptor agonist, Bcl-xL Bcl-2 associated death promotor modulators, beta amyloid antagonist, beta-catenin inhibitors, bromodomain containing protein inhibitor, Ca2+ release activated Ca2+ channel 1 inhibitors, calcineurin inhibitors, calcium channel inhibitors, cannabinoid CB1 receptor antagonist, cathepsin S inhibitors, CCR3 chemokine antagonists, CXCR2 chemokine antagonist, CXCR1/2 chemokine, CCR6 chemokine antagonist, CD223 modulators, CD40 ligand receptor antagonists, cell adhesion molecule inhibitors, cell surface glycoprotein MUC18 inhibitors, CREB binding protein inhibitors, CXCR4 chemokine modulators, cytokine receptor antagonist, cytosolic phospholipase A2 inhibitors, DHFR inhibitors, DYRK-1 alpha protein kinase inhibitor, EGFR family tyrosine kinase receptor inhibitors, enolase 1 inhibitor, eotaxin ligand inhibitors, F1F0 ATP synthase modulator, free fatty acid receptor 2 agonist, free fatty acid receptor 3 agonist, galectin-3 inhibitors, glucocorticoid agonists, GM-CSF ligand inhibitors, GNRH receptor modulators, 5-HT 1a receptor antagonist, FGF receptor antagonist, GroEL protein 2 inhibitor, histamine H1 receptor antagonists, histamine H4 receptor antagonists, histone deacetylase-1 inhibitors, histone deacetylase-2 inhibitors, histone deacetylase-3 inhibitors, histone deacetylase-6 inhibitors, Hsp 90 inhibitor, IL-1 receptor antagonist, interleukin 1 like receptor 2 inhibitor, IL-2 receptor alpha subunit stimulator, IL-2 modulator, IL-10 antagonists, IL-12 antagonists, IL-17 agonist, IL17RA gene inhibitor, IL-17 antagonists, IL-23 antagonists, IL-8 antagonists, immunoglobulin like domain receptor 2 antagonist, insulin receptor substrate-1 inhibitors, interferon gamma receptor antagonists, interleukin 17 ligand inhibitors, interleukin 17A ligand inhibitors, interleukin 17A ligand modulators, interleukin 17F ligand inhibitors, interleukin 23A inhibitors, interleukin receptor 17A antagonists, interleukin receptor 17A modulators, interleukin-1 alpha ligand inhibitors, interleukin-1 beta ligand modulators, IRAK-4 protein kinase inhibitor, Itk tyrosine kinase inhibitor, JAK tyrosine kinase inhibitors, Jak1 tyrosine kinase inhibitors, Jak2 tyrosine kinase inhibitors, Jak3 tyrosine kinase inhibitors, KCNA voltage-gated potassium channel-3 inhibitors, Lck tyrosine kinase inhibitors, lysophosphatidate-1 receptor antagonists, MALT protein 1 inhibitors, MAP kinase inhibitors, membrane copper amine oxidase inhibitors, metalloprotease-1 inhibitors, mitochondrial 10 kDa heat shock protein stimulators, mTOR complex 1 inhibitor, mTOR complex 2 inhibitor, non receptor tyrosine kinase TYK2 antagonists, nuclear erythroid 2-related factor 2 stimulators, nuclear factor kappa B inhibitors, nucleoside reverse transcriptase inhibitors, oncostatin M receptor subunit beta inhibitor, opioid receptor delta antagonists, OX40 ligand inhibitor, parathyroid hormone ligand inhibitors, PDE 4 inhibitors, PDE 4b inhibitor, P2Y6 purinoceptor modulator; P-glycoprotein inhibitors, phosphoinositide-3 kinase delta inhibitors, phosphoinositide-3 kinase gamma inhibitors, phospholipase A2 inhibitors, programmed cell death ligand 1 modulators, programmed cell death protein 1 stimulator, P-selectin glycoprotein ligand-1 stimulators, retinoic acid receptor agonists, retinoic acid receptor gamma antagonists, retinoic acid receptor gamma inverse agonists, retinoid receptor agonists, retinoid X receptor agonists, retinoid X receptor modulators, retinoid Z receptor gamma agonists, retinoid Z receptor gamma inverse agonists, retinoid Z receptor gamma antagonist, rho associated protein kinase 2 inhibitors, ribonuclease P inhibitors, RIP-1 kinase inhibitor, sphingosine-1-phosphate receptor-1 antagonists, sphingosine-1-phosphate receptor-1 modulators, Src tyrosine kinase inhibitors, STAT-3 inhibitors, Syk tyrosine kinase inhibitor, T-box transcription factor TBX21 modulators, T-cell differentiation antigen CD6 inhibitors, T-cell surface glycoprotein CD8 inhibitors, T cell surface glycoprotein CD28 stimulator, TGF beta agonists, TLR-7 antagonists, TLR-8 antagonists, TLR-9 antagonists, TNF alpha ligand inhibitors, TNF alpha ligand modulators, TNF antagonists, TNF binding agents, TNF gene inhibitor, topoisomerase II inhibitors, TrkA receptor antagonists, tubulin binding agents, Tyk2 tyrosine kinase inhibitor, type II TNF receptor modulators, unspecified cytokine receptor antagonists, vitamin D3 receptor agonists, vitamin D3 receptor modulators, Wnt ligand inhibitor, and Wnt 5A ligand inhibitor.

In some embodiments, the compounds provided herein, or pharmaceutically acceptable salts thereof, may be combined with one, two, three, or four additional therapeutic agents selected from AP-005, 18C3 (anti-IL-1 alpha true human antibody), ABX-464, acitretin, adalimumab, adipocell, AFB-035, aganirsen, AKP-11, alefacept, alitretinoin, Amilo-5mer, aminopterin, amiselimod, apremilast, ASKP-1240, AST-005, ATI-2138, AVX-001, baricitinib, belapectin (GR-MD-02), bertilimumab, betamethasone, BI-655066, BI-730357, BI-730460, BI-730460, bimekizumab, BMS-986165, BMX-010, briakinumab, brodalumab, BTT-1023, C-82, calcipotriol, calcitriol, CC-90005, CCL-20LD, CD-10367, certolizumab pegol, CF-101, ciclosporin, CJM-112, CKBA, clobetasol propionate+tretinoin, CM-2489, CPL-409116, crisaborole, CS-12192, CT-327, CTX-101, dalazatide, DFD-06, dimethyl fumarate, dithranol, DLX-105, DSXS-1411, DSXS-1535, DUR-928, EDP-1815, etanercept, fluocinonide, FPP-003, GK-664-S, GLG-801, GLPG-3121, GLPG-3667, GLPG-3970, GLY-2028, GMDP, GSK-2800528, GSK-2831781, GSK-2981278A, guselkumab, halomethasone, HAT-1, IMO-3100, IMO-8400, inecalcitol, infliximab, INV-103, IR-444, IR-502, itolizumab, ixekizumab, JN-2528, KBL-697, KD-025, LAS-41004, LEO-124249, LEO-29102, LEO-32731, LEO-35299, lithium succinate, LNP-1955, LP-0200, M-1095, maxacalcitol, MDX-018, methotrexate, MOL-4249, mometasone, MP-1032, MSB-03, myristyl nicotinate, namilumab, neihulizumab, niclosamide, NLP-91, NP-000888, NVN-1000, olopatadine, orilotimod, P-3072, P-3073, PAT-1657, Pc4, pefcalcitol, PF-06700841, Prurisol, PRX-003, PRX-167700, PUR-0110, recombinant human LFA-3/antibody fusion protein, RON-2315, RTU-1096, S-414114, secukinumab, SHP-141, SMET-D1, SNK-01, SP-14019, SSS-07, tacalcitol, tazarotene, tildrakizumab, tirbanibulin (KX-01), tofacitinib, toreforant, tregalizumab, TU-2100, UCB-5857, UHE-105, ulobetasol, ustekinumab, VBY-891, voclosporin, VTP-43742, WBI-1001, and ZPL-389, or a pharmaceutically acceptable salt of any of the foregoing, or any combination thereof.

In some embodiments, the compounds provided herein, or pharmaceutically acceptable salts thereof, may be combined with one, two, three, or four additional therapeutic agents selected from:
acetaldehyde dehydrogenase inhibitor, including but not limited to ADX-629;
adenosine A3 receptor agonists, including but not limited to piclidenoson (CF-101);
adenosine A3 receptor antagonist, including but not limited to PBF-1650;
ADP ribosyl cyclase-1 inhibitors, including but not limited to IMO-3100;
5-HT 1a receptor antagonist, including but not limited to AX-1602;
apolipoprotein A antagonist, including but not limited to orticumab;
cytokine receptor antagonist, including but not limited to tapinarof;
aryl hydrocarbon receptor modulator, including but not limited to NTI-528 and RLV-102;
Bcl-xL Bcl-2 associated death promotor modulators, including but not limited to Pc4;
beta-catenin inhibitors, including but not limited to C-82;
bromodomain containing protein inhibitor, including but not limited to BOS-475;
Ca2+ release activated Ca2+ channel 1 inhibitors, including but not limited to CM-2489 and PRCL-02;
calcineurin inhibitors, including but not limited to voclosporin, pimecrolimus, tacrolimus, ciclosporin, HS-378, oxeclosporin, OLO-400, ADV-P3, and CTX-006;
calcium channel inhibitors, including but not limited to RP-3128;
cathepsin S inhibitors, including but not limited to VBY-129, VBY-891, RWJ-445380, and CRA-028129;
CCR3 chemokine antagonists, including but not limited to bertilimumab;
CXCR2 chemokine antagonist, including but not limited to CCX-624;
CD223 modulators, including but not limited to GSK-2831781;
CD40 ligand receptor antagonists, including but not limited to ASKP-1240, lucatumumab, and toralizumab;
cell adhesion molecule inhibitors, including but not limited to BIRT-2584, PC-114, alicaforsen, IC-747, ICM-3, and ISIS-2302;
cell surface glycoprotein MUC18 inhibitors, including but not limited to PRX-003 and imaprelimab;
CREB binding protein inhibitors, including but not limited to C-82;
CXCR1/2 chemokine, including but not limited to LY-3041658;
CXCR4 chemokine modulators, including but not limited to CD184-FK506 ADC;
cytosolic phospholipase A2 inhibitors, including but not limited to AVX-001;
DHFR inhibitors, including but not limited to methotrexate, CH-4051, CePep, CH-1504, MQX-5902, and MPI-2505;
DYRK-1 alpha protein kinase inhibitor, including but not limited to VRN-02;
EGFR family tyrosine kinase receptor inhibitors, including but not limited to erlotinib, icotinib hydrochloride, and SGT-210;
Enolase 1 inhibitor, including but not limited to HuL-001;
Eotaxin ligand inhibitors, including but not limited to bertilimumab;
F1F0 ATP synthase modulator, including but not limited to LYC-30937;
FGF receptor antagonist, including but not limited to potassium dobesilate;
free fatty acid receptor 2, 3 agonist, including but not limited to SFA-002;
galectin-3 inhibitors, including but not limited to belapectin (GR-MD-02);
glucocorticoid agonists, including but not limited to betamethasone, clobetasol, auranofin, NM-135, DSXS-1538b, and SEGRA;
GM-CSF ligand inhibitors, including but not limited to namilumab;
GNRH receptor modulators, including but not limited to NL-001;
GroEL protein 2 inhibitor, including but not limited to prozumab;
histamine H1 receptor antagonists, including but not limited to olopatadine and loratadine+nortriptyline;
histamine H4 receptor antagonists, including but not limited to toreforant and ZPL-389;
histone deacetylase-2 inhibitors, including but not limited to KAR-1880;
histone deacetylase 1, 6, 2, 3 inhibitors, including but not limited to remetinostat (SHP-141);
Hsp 90 inhibitor, including but not limited to CTXT-102;
IL-2 receptor alpha subunit stimulator, including but not limited to NKTR-358;
IL-2 modulator; including but not limited to CC-92252;
IL-10 antagonists, including but not limited to pimecrolimus;
IL-12 antagonists, including but not limited to BOW-090, briakinumab, FM-202, and apilimod;
IL-17 antagonists, including but not limited to ixekizumab, secukinumab, AFB-035, KD-025, DLX-3003, EBI-028, M-1095, IMO-3100, GR-1501, 608, vunakizumab, sonelokimab, AK-111, HB-0017, and SIM-335;
IL-17 agonist, including but not limited to ZL-1102;
IL17RA gene inhibitor, including but not limited to XCUR-17;
IL-23 antagonists, including but not limited to tildrakizumab, BI-655066, AMG-139, briakinumab, mirikizumab (LY-3074828), FM-202, apilimod, LY-2525623, risankizumab, and IBI-112;
IL-23 antagonist, including but not limited to ustekinumab and AK-101;
IL-8 antagonists, including but not limited to BMS-986253 (MDX-018), AS-101, ABX-IL8, LI-312, SB-332235, and LF-216;
immunoglobulin like domain receptor 2 antagonist, including but not limited to CGEN-15001;
insulin receptor substrate-1 inhibitors, including but not limited to aganirsen;
interferon gamma receptor antagonists, including but not limited to pimecrolimus, AMG-811, OA-1, AGT-1, mometasone+nortriptyline, and fontolizumab;
interleukin 17 ligand inhibitors, including but not limited to CJM-112, netakimab, and AFB-035;
interleukin 17A ligand inhibitors, including but not limited to COVA-322, JS-005, and ABY-035/AFO2;
interleukin 17A ligand modulators, including but not limited to QX-002-N;
interleukin 17A/17F ligand inhibitors, including but not limited to bimekizumab;
interleukin 23A inhibitors, including but not limited to guselkumab and QX-004-N;
interleukin receptor 17A antagonists, including but not limited to brodalumab and LZM-012;
interleukin 1 like receptor 2 inhibitor, including but not limited to spesolimab and imsidolimab;
interleukin-1 alpha ligand inhibitors, including but not limited to bermekimab (CA-18C3);
interleukin-1 beta ligand modulators, including but not limited to PUR-0110 and AR-100;
IRAK-4 protein kinase inhibitor, including but not limited to BAY-1834845;
Itk tyrosine kinase inhibitor, including but not limited to JTE-051;
JAK tyrosine kinase inhibitors, including but not limited to CS-17380;
Jak1 tyrosine kinase inhibitors, including but not limited to itacitinib, abrocitinib (PF-04965842), solcitinib, SHR-0302, and filgotinib;
JAK1,2,3 tyrosine kinase inhibitor, including but not limited to jaktinib;
JAk1,2 tyrosine kinase inhibitor, including but not limited to baricitinib and ruxolitinib;
TYk2 tyrosine kinase inhibitor, including but not limited to brepocitinib;
Jak1 tyrosine kinase inhibitor, including but not limited to PF-06263276;
JAk 1, 3 tyrosine kinase inhibitor, including but not limited to CS-944X, tofacitinib, and peficitinib;
KCNA voltage-gated potassium channel-3 inhibitors, including but not limited to KPI-150, dalazatide, BNC-164, and SPS-4251;
Lck tyrosine kinase inhibitors, including but not limited to BMS-350751 and NTRC-0625-0;
lysophosphatidate-1 receptor antagonists, including but not limited to BMS-986202;
MAP kinase inhibitors, including but not limited to AIK-33 and KIN-3032;
membrane copper amine oxidase inhibitors, including but not limited to vepalimomab, BTT-1023, RTU-1096, and PRX-167700;
metalloprotease-1 inhibitors, including but not limited to KIN-3032 and HMR-1571;
mitochondrial 10 kDa heat shock protein stimulators, including but not limited to INV-103;
Non receptor tyrosine kinase TYK2 antagonists, including but not limited to SAR-20347, ICP-332, and SDC-1801;
nuclear erythroid 2-related factor 2 stimulators, including but not limited to dimethyl fumarate and XP-23829;
nuclear factor kappa B inhibitors, including but not limited to S-414114, VGX-1027, AKBA, SP-100030, and YP-008;
nucleoside reverse transcriptase inhibitors, including but not limited to Prurisol;
oncostatin M receptor subunit beta inhibitor, including but not limited to vixarelimab;
Opioid receptor delta antagonists, including but not limited to HS-378;
OX40 ligand inhibitor, including but not limited to KY-1005;
P38 MAP kinase inhibitor, including but not limited to AMG-101, AIK-3, VGX-1027, AIK-a1, BMS-582949, doramapimod, semapimod, TA-5493, HEP-689, and RWJ-68354;
parathyroid hormone ligand inhibitors, including but not limited to inecalcitol;
PDE 4 inhibitors, including but not limited to apremilast, roflumilast, orismilast, MK-0873, Ro-20-1724, HMR-1571, RPR-122818, HPP-737, crisaborole, and DC-591042;
PDE 4b inhibitor, including but not limited to GRT-6015;
TNF alpha ligand inhibitor, including but not limited to Hemay-005;
P-Glycoprotein inhibitors, including but not limited to boningmycin;
Beta amyloid antagonist, including but not limited to GC-021109;
phosphoinositide-3 kinase delta inhibitors, including but not limited to seletalisib (UCB-5857);
mTOR complex 2 inhibitor, including but not limited to bimiralisib;
phosphoinositide-3 kinase gamma inhibitors, including but not limited to TAT-N25 peptide;
phospholipase A2 inhibitor, including but not limited to ZPL-521, Project P-0229, BMS-181162, and BMS-188184;
programmed cell death ligand 1 modulators, including but not limited to GX-P2;
programmed cell death protein 1 stimulator, including but not limited to LY-3462817 and CC-90006;
P-selectin glycoprotein ligand-1 stimulators, including but not limited toneihulizumab;
P-selectin glycoprotein ligand-1, including but not limited to AbGn-168H;
retinoic acid receptor agonists, including but not limited to acitretin, tazarotene, tretinoin, tazarotene arotinoid trometamol, CD-1599, AM-580, BMS-181163, and CPR-2005;
retinoic acid receptor gamma antagonists, including but not limited to VTP-43742 and BBI-6000;
retinoic acid receptor gamma inverse agonists, including but not limited to GSK-2981278A and JNJ-3534;
retinoid receptor agonists, including but not limited to RASP;
retinoid X receptor agonists, including but not limited to LGD-1550;
retinoid X receptor modulators, including but not limited to bexarotene, alitretinoin, ALRT-1069, LGD-1069, and Net-41B;
retinoid Z receptor gamma agonists, including but not limited to NCE-407;
retinoid Z receptor gamma inverse agonists, including but not limited to ARN-6039, IMU-935, BOS-172767, SAR-441169, and INV-17;
retinoid Z receptor gamma antagonist, including but not limited to AUR-101, JTE-451, ESR-114, ABBV-157, and AZD-0284;
rho associated protein kinase 2 inhibitors, including but not limited to KD-025;
RIP-1 kinase inhibitor, including but not limited to GSK-2982772, DNL-758, and VRN-04;
ribonuclease P inhibitors, including but not limited to RASP;
sphingosine-1-phosphate receptor-1 modulators, including but not limited to amiselimod, AKP-11, FP-253, and CS-0777;
sphingosine-1-phosphate receptor-1 agonist, including but not limited to AK-119, SCD-044, and SYL-927;
sphingosine-1-phosphate receptor-5 modulator, including but not limited to CBP-307;
Src tyrosine kinase inhibitors, including but not limited to tirbanibulin (KX-01);
STAT-3 inhibitors, including but not limited to TAK-114, GLG-801, and MOL-4249;
Syk tyrosine kinase inhibitor, including but not limited to HMPL-523;
T-box transcription factor TBX21 modulators, including but not limited to SB-020;
T-cell differentiation antigen CD6 inhibitors, including but not limited to itolizumab;
T-cell surface glycoprotein CD8 inhibitors, including but not limited to tregalizumab;
T cell surface glycoprotein CD28 stimulator, including but not limited to theralizumab;
TGF beta agonists, including but not limited to tregalizumab;
TLR-7 antagonists, including but not limited to IMO-3100;
TLR-9 antagonists, including but not limited to IMO-3100 and GNKS-356;
TLR 7,8,9 antagonist, including but not limited to IMO-8400;
TNF alpha ligand inhibitors, including but not limited to adalimumab, CHS-1420, BAX-2923, MSB-11022, ABP-501, MYL-1401A, infliximab, certolizumab pegol, AST-005, etanercept, opinercept, ISIS-104838, DLX-105, SSS-07, DLX-2751, DLX-105, Debio-0512, TAQ-588, adalimumab, placulumab, PMI-001, CYT-020-TNFQb, AN-0128, CYT-007-TNFQb, SYI-2074, YP-008, SCT-640A, SBT-104, and T-1649;
TNF alpha ligand modulators, including but not limited to PUR-0110, CDP-571, and ACU-D2;
TNF antagonists, including but not limited to certolizumab pegol, SCB-808, BAX-2200, CT-P05, SCB-131, GSK-2800528, onercept, and ALS-00T2-0501;
TNF binding agents, including but not limited to adalimumab, certolizumab pegol SCB-131, onercept, CT-P17, SBC-808, ABP-501, MYL-1401A, MSB-11022, BAX-2923, CHS-1420, and BCD-057;
TNF gene inhibitor, including but not limited to AST-005;
topoisomerase II inhibitors, including but not limited to GPX-150;
TrkA receptor antagonists, including but not limited to VM-902A, CT-327, K-252a, and lestaurtinib;
tubulin binding agents, including but not limited to KX-01 and paclitaxel;
Tyk2 tyrosine kinase inhibitor, including but not limited to deucravacitinib, PF-06826647, ABBV-712, and CS-43001;
type II TNF receptor modulators, including but not limited to TNR-001, BAX-2200, and SCB-131;
unspecified cytokine receptor antagonists, including but not limited to tetrathiomolybdate, JD-4000, X-083-NAB, SPHD-400, pimecrolimus, and HMPL-010;
vitamin D3 receptor agonists, including but not limited to inecalcitol, maxacalcitol, calcipotriol, falecalcitriol, maxacalcitol, calcitriol NS-78, tacalcitol, calcipotriol, calcithiazol, ecalcidene, lexacalcitol, atocalcitol, and Ro-65-2299;
vitamin D, D3 receptor modulators, including but not limited to VS-320 and VS-105;
Wnt ligand inhibitor, including but not limited to SM-04755; and
Wnt 5A ligand inhibitor, including but not limited to Box-5.

### Rheumatoid Arthritis Combination Therapy

In some embodiments, the compounds provided herein, or pharmaceutically acceptable salts thereof, may be combined with one, two, three, or four additional therapeutic agents that are useful for treating or ameliorating rheumatoid arthritis. In some embodiments, the compounds provided herein, or pharmaceutically acceptable salts thereof, may be combined with one, two, three, or four additional therapeutic agents selected from 14-3-3 protein eta inhibitors, 5-lipoxygenase inhibitors, abl tyrosine kinase inhibitors, ACTH receptor agonists, adenosine A3 receptor agonists, adenosine deaminase inhibitors, ADP ribosyl cyclase-1 inhibitors, ADP ribosyl cyclase-1 modulators, ADP ribosylation factor 6 inhibitors, adrenocorticotrophic hormone ligands, aggrecanase-2 inhibitors, albumin modulators, anti-TNF steroid conjugate, adenosine A1 receptor antagonist, annexin A1 modulator, API transcription factor inhibitors, apolipoprotein B modulator, aryl hydrocarbon receptor agonist plus autoantigen, basigin inhibitors, bcr protein inhibitors, B-lymphocyte antigen CD19 inhibitors, B-lymphocyte antigen CD20 inhibitors, B-lymphocyte antigen CD20 modulators, B-lymphocyte cell adhesion molecule inhibitor, B-lymphocyte stimulator ligand inhibitors, bradykinin receptor modulators, BRAF gene inhibitors, branched amino acid aminotransferase 1 inhibitors, bromodomain containing protein inhibitors, Btk tyrosine kinase inhibitors, cadherin-11 antagonists, calcineurin inhibitors, calcium channel inhibitors, calreticulin inhibitor, carbonic anhydrase inhibitors, cathepsin K inhibitors, cathepsin S inhibitors, CCR1 chemokine antagonists, CCR2 chemokine antagonists, CCR3 gene modulators, CCR5 chemokine antagonists, CD126 antagonists, CD29 modulators, CD3 modulators, CD39 agonists, CD4 agonists, CD4 antagonists, CD40 ligand inhibitors, CD40 ligand receptor antagonists, CD40 ligand receptor modulators, CD52 antagonists, CD73 agonists, CD79b modulators, CD80 antagonists, CD86 antagonists, CD95 antagonists, cell adhesion molecule inhibitors, chaperonin modulator, choline kinase inhibitors, clusterin stimulators, complement C5 factor inhibitors, complement factor stimulators, C-reactive protein inhibitors, CSF-1 antagonists, CXC10 chemokine ligand inhibitors, CXCR4 chemokine antagonists, cy clin-dependent kinase inhibitor 1 inhibitors, cyclin-dependent kinase-2 inhibitors, cyclin-dependent kinase-4 inhibitors, cyclin-dependent kinase-5 inhibitors, cyclin-dependent kinase-6 inhibitors, cyclin-dependent kinase-7 inhibitors, cyclin-dependent kinase-9 inhibitors, cyclooxygenase 2 inhibitors, cyclooxygenase 2 modulators, cyclooxygenase inhibitors, cytosolic phospholipase A2 inhibitors, cytotoxic T-lymphocyte protein-4 modulators, cytotoxic T-lymphocyte protein-4 stimulators, deoxyribonuclease gamma stimulator, DHFR inhibitors, diamine acetyltransferase inhibitors, dihydroorotate dehydrogenase inhibitors, DYRK-1 alpha protein kinase inhibitor, elongation factor 2 inhibitors, enolase 1 inhibitor, eotaxin 2 ligand inhibitors, EP4 prostanoid receptor antagonists, erythropoietin receptor agonists, factor XIIa antagonist, Fas ligands, FGF-2 ligand inhibitors, FK506 binding protein-12 modulators, folate antagonists, folate receptor agonists, folate receptor beta antagonists, folate receptor modulators, fractalkine ligand inhibitors, fyn tyrosine kinase inhibitors, G protein coupled receptor 15 antagonists, GABA A receptor modulators, glucocorticoid agonists, glucocorticoid antagonists, glucocorticoid induced leucine zipper stimulators, GM-CSF ligand inhibitors, GM-CSF receptor antagonists, GM-CSF receptor modulators, growth regulated protein alpha ligand inhibitors, H+K+ ATPase inhibitors, histamine H4 receptor antagonists, histone deacetylase inhibitors, histone deacetylase-6 inhibitors, HIV-1 gp120 protein inhibitors, HLA class II antigen DQ-2 alpha modulators, HLA class II antigen inhibitors, HLA class II antigen modulators, Hsp 70 family inhibitors, hypoxia inducible factor-1 inhibitors, IFNB gene stimulators, I-kappa B kinase beta inhibitors, I-kappa B kinase inhibitors, IL-1 antagonists, IL-10 agonists, IL-11 agonists, IL-12 antagonists, IL-15 antagonists, IL-17 antagonists, IL-17 receptor modulators, IL-18 receptor accessory protein antagonist, IL-8 ligand inhibitors, IL-2 agonists, IL-2 antagonists, IL-21 antagonists, IL-23 antagonists, IL-3 antagonists, IL-4 agonists, IL-6 antagonists, IL-6 receptor modulators, IL-6 neutralizing human antibodies, anti-IL6 antibody, immunoglobulin antagonists, immunoglobulin G1 agonists, immunoglobulin G1 antagonists, immunoglobulin G1 modulators, immunoglobulin G2 antagonists, immunoglobulin G2 modulators, immunoglobulin gamma Fc receptor II modulators, immunoglobulin gamma Fc receptor IIB antagonists, immunoglobulin kappa modulators, immunoglobulin M antagonists, inducible nitric oxide synthase inhibitors (iNOS inhibitors), inosine monophosphate dehydrogenase inhibitors, insulin sensitizers, integrin alpha-1/beta-1 antagonists, integrin alpha-4/beta-1 antagonists, integrin alpha-9 antagonist, integrin antagonists, interferon beta ligands, interferon gamma ligands, interleukin 17A ligand inhibitors, interleukin 17F ligand inhibitors, interleukin 23A inhibitors, interleukin ligands, interleukin receptor 17A antagonists, interleukin-1 betaligand inhibitors, interleukin-10 ligands, interleukin-2 ligands, interleukin-4 ligands, Interleukin-6 ligand inhibitors, Itk tyrosine kinase inhibitors, JAK tyrosine kinase inhibitors, Jak1 tyrosine kinase inhibitors, Jak2 tyrosine kinase inhibitors, JAK3 gene inhibitors, Jak3 tyrosine kinase inhibitors, Jun N terminal kinase inhibitors, KCNA voltage-gated potassium channel-3 modulators, kelch like ECH associated protein 1 modulators, kit tyrosine kinase inhibitors, LanC like protein 2 modulators, leukotriene BLT receptor antagonist, LITAF gene inhibitors, lymphocyte function antigen-3 receptor antagonists, Lyn tyrosine kinase inhibitors, macrophage-drug conjugate (MDC), macrophage mannose receptor 1 modulators, MAdCAM inhibitors, MAP kinase modulators, MAP3K2 gene inhibitors, MAPKAPK5 inhibitors, matrix metalloprotease inhibitors, MCL1 gene inhibitors, MEK protein kinase inhibitors, MEK-1 protein kinase inhibitors, MEK-2 protein kinase inhibitors, membrane copper amine oxidase inhibitors, metalloprotease-2 inhibitors, metalloprotease-9 inhibitors, methylprednisolone, midkine ligand inhibitors, mitochondrial 10 kDa heat shock protein stimulators, mTOR complex 1 inhibitors, mTOR inhibitors, NAD ADP ribosyltransferase stimulators, NAMPT gene inhibitors, NF kappaB inhibitor stimulators, NFAT gene inhibitors, NFE2L2 gene stimulators, nicotinic acetylcholine receptor antagonists, NK cell receptor modulators, NKG2 A B activating NK receptor antagonists, NKG2 D activating NK receptor antagonists, nuclear erythroid 2-related factor 2 stimulators, nuclear factor kappa B inhibitors, nuclear factor kappa B modulators, nuclear factor kappa B p105 inhibitors, opioid growth factor receptor agonists, opioid receptor delta antagonists, osteoclast differentiation factor antagonists, osteoclast differentiation factor ligand inhibitors, oxidoreductase inhibitors, P2X7 purinoceptor agonists, p38 MAP kinase alpha inhibitors, p38 MAP kinase inhibitors, PDE 4 inhibitors, PDE 5 inhibitors, PDGF receptor agonists, PDGF receptor antagonists, PDGF-B ligand inhibitors, PERK gene inhibitors, phosphoinositide-3 kinase delta inhibitors, phosphoinositide-3 kinase gamma inhibitors, phospholipase A2 inhibitors, platelet activating factor receptor antagonists, PPAR gamma agonists, programmed cell death protein 1 modulators, prostaglandin D synthase stimulators, protein arginine deiminase inhibitors, protein tyrosine kinase inhibitors, protease-activated receptor-2 antagonist, PurH purine biosynthesis protein inhibitors, rho associated protein kinase 2 inhibitors, seprase inhibitors, signal transducer CD24 modulators, signal transduction inhibitors, sodium glucose transporter-2 inhibitors, sphingosine 1 phosphate phosphatase modulators, STAT3 gene inhibitors, serum amyloid A protein modulator, superoxide dismutase stimulators, SYK family tyrosine kinase inhibitors, Syk tyrosine kinase inhibitors, syndecan-1 inhibitors, T cell receptor antagonists, T cell receptor modulators, T cell surface glycoprotein CD28 inhibitors, T cell surface glycoprotein CD28 stimulators, TAK1 binding protein modulators, talin modulators, T-cell differentiation antigen CD6 inhibitors, T-cell surface glycoprotein CD8 inhibitors, tenascin modulators, TGF beta agonists, thymulin agonists, TLR-2 antagonists, TLR-4 antagonists, TLR-9 antagonists, TNF alpha ligand inhibitors, TNF alpha ligand modulators, TNF antagonists, TNF gene inhibitors, TNF receptor modulators, TNFSF11 gene inhibitors, transcription factor p65 inhibitors, transcription factor RelB inhibitors, transferrin modulators, transthyretin modulator, tumor necrosis factor 13C receptor antagonists, tumor necrosis factor 15 ligand inhibitors, tumor necrosis factor ligand 13 inhibitors, tumor necrosis factor ligand inhibitors, type I IL-1 receptor antagonists, type I TNF receptor antagonists, type II TNF receptor modulators, unspecified GPCR agonists, VEGF receptor antagonists, VEGF-2 receptor antagonists, VEGF-2 receptor modulators, VEGF-B ligand inhibitors, X-linked inhibitor of apoptosis protein inhibitors, and zap70 tyrosine kinase inhibitors.

In some embodiments, the compounds provided herein, or pharmaceutically acceptable salts thereof, may be combined with one, two, three, or four additional therapeutic agents selected from 99mTc labelled annexin V-128, abatacept, abatacept biosimilar, ABBV-257, ABT-122, ABT-494, acalabrutinib, aceclofenac, actarit, AdMSCs, MS-392, adalimumab, adalimumab biosimilar, adalimumab follow-on biologic, AK-106, ALX-0061, Amilo-5MER, aminopterin, AMT-101, anakinra, anakinra biosimilar, anakinra follow-on biologic, annexuzlimab, ARG-301, ARQ-250, ASLAN-003, ASP-5094, AT-132, AZD-9567, baricitinib, BI-655064, bimekizumab, BiP (rheumatoid arthritis), BLHP-006, blisibimod, BMS-986104, BMS-986142, ABBV-105, BTT-1023, canakinumab, Cartistem, CCX-354, CD24-IgFc, celecoxib, cerdulatinib, certolizumab pegol, CF-101, CFZ-533, CHR-5154, cibinetide, ciclosporin, clazakizumab, CNTO-6785, corticotropin, CR-6086, CreaVax-RA, CWG-92, CWG-940, Cx-611, DE-098, DEN-181, deflazacort, Rheumavax, denosumab, diacerein, diclofenac, DWJ-1421, E-6011, eicosapentaenoic acid monoglycerides, etanercept, etanercept biosimilar, etanercept follow-on biologic, etodolac, etoricoxib, filgotinib, fosdagrocorat, GLPG-3970, gerilimzumab, ginsenoside C—K, givinostat, GLPG-4399, goat polyclonal antibodies, golimumab, GS-5745, GS-9876, GSK-3196165, HHT-109, HM-71224, HMPL-523, HST-003, hyaluronate sodium, (S)-hydroxychloroquine, IB-RA (injectable, rheumatoid arthritis), IB-RA (oral, rheumatoid arthritis), IcanoMAB, ICP-022, iguratimod, IMD-2560, imidazole salicylate, infliximab, infliximab biobetter, infliximab biosimilar, CT-P13, INSIX RA, interferon gamma follow-on biologic, interleukin-2 (injectable), interleukin-2 follow-on biologic, INV-103, IR-501, itolizumab, JNJ-40346527, Ka Shu Ning, KB-312, KD-025, ketoprofen+omeprazole, KINE-101, LB-600, leflunomide, lenzilumab, LLDT-8, LNK-01001, LNP-1955, lumiracoxib, LY-3090106, masitinib, mavrilimumab, MBS-2320, MEDI-5117, meloxicam, methotrexate, MGD-010, misoprostol+diclofenac, MM-A01-01, monalizumab, MORAb-022, MPC-300-IV, MRC-375, nabumetone, namilumab, naproxen+esomeprazole, naproxen+esomeprazole strontium, NIP-046, ocaratuzumab, ofatumumab, OHR-118, olokizumab, OM-89, once-daily naproxen (oral controlled release, pain), ONO-4059, Oralgam, ozoralizumab, PAR-2 inhibitors, peficitinib, pelubiprofen, PF-06687234, piperidone hydrochloridum, piroxicam, prednisolone, prednisone, Procell, Prosorba, PRT-2607, PRTX-100, PRX-167700, QBSAU, rabeximod, RCT-18, recombinant human CD22 monoclonal antibody (iv infusion), Lonn Ryonn Pharma/SinoMab Bioscience (Shenzhen), RA-Curcusome, recombinant human interleukin-1 receptor antagonist (rheumatoid arthritis), recombinant human interleukin-2 recombinant TNF receptor 2-Fc fusion protein mutant, RG-6125, RhuDex, rifabutin+clarithromycin+clofazimine, rituximab, rituximab biosimilar, Toritz, rituximab follow-on biologic, RPI-78, SAN-300, sarilumab, SBI-087, seliciclib, SHR-0302, sirukumab, spebrutinib, SR-047, SSS-07, KDDF-201110-06, Syn-1002, T-5224, TAB-08, tacrolimus, TAK-020, TAK-079, tarenflurbil (transdermal spraygel, skin disease/rheumatoid arthritis), technetium Tc 99m tilmanocept, technetium[99Tc] methylenediphosphonate, tenoxicam, Debio-0512, tocilizumab, tofacitinib, tofacitinib citrate, TQG-2813, Trichuris suis ova, umbilical cord-derived mesenchymal stem cells (iv, RA/liver disease), ustekinumab, VAY-736, VB-201, WF-10, XmAb-5871, YH-1713, YHB-1411-2, YRA-1909, and ZM-008, or a pharmaceutically acceptable salt of any of the foregoing, or any combination thereof.

In some embodiments, the compounds provided herein, or pharmaceutically acceptable salts thereof, may be combined with one, two, three, or four additional therapeutic agents selected from:
14-3-3 protein eta inhibitors, including but not limited to anti-AGX-020 mAbs (rheumatoid arthritis) and Augurex;
5-Lipoxygenase inhibitors, including but not limited to darbufelone, tebufelone, ZD-2138, etalocib, PGV-20229, L-708780, T-0757, T-0799, ZM-216800, L-699333, BU-4601A, and SKF-104351;
5-Lipoxygenase/Cyclooxygenase inhibitors, including but not limited to tenoxicam, licofelone, tenidap, tepoxalin, flobufen, SKF-86002, WY-28342, and CI-986;
5-Lipoxygenase/PPAR gamma agonists, including but not limited to etalocib;
Abl tyrosine kinase inhibitors/Bcr protein inhibitors/Kit tyrosine kinase inhibitors/PDGF receptor antagonists/Signal transduction inhibitors, including but not limited to imatinib;
ACTH receptor agonists/Adrenocorticotrophic hormone ligands/Opioid growth factor receptor agonists, including but not limited to FAR-404 and metenkefalin acetate+tridecactide acetate;
adenosine A1 receptor antagonist, including but not limited to CP-25;
adenosine A3 receptor agonists, including but not limited to CF-101 (piclidenoson);
adenosine deaminase inhibitors, cladribine, pentostatin, and FR-221647;
ADP ribosyl cyclase-1 inhibitors, including but not limited to daratumumab;
ADP ribosyl cyclase-1 modulators/syndecan-1 inhibitors, including but not limited to indatuximab ravtansine;
ADP ribosylation factor 6 inhibitors, including but not limited to NAV-2729;
adrenocorticotrophic hormone ligands, including but not limited to corticotropin and Mallinckrodt;
aggrecanase-2/TNF gene inhibitors, including but not limited to GIBH-R-001-2;
albumin modulators, including but not limited to ONS-1210;
albumin modulators/IL-6 antagonists, including but not limited to ALX-0061 (vobarilizumab);
albumin modulators/TNF alpha ligand inhibitors, including but not limited to HOT-3010;
API transcription factor/nuclear factor kappa B inhibitors, including but not limited to tarenflurbil and SP-100030;
anti-TNF steroid antibody-drug conjugates (anti-TNF-GRM), including but not limited to ABBV-3373 and ABBV-154;
basigin inhibitors/branched amino acid aminotransferase 1/metalloprotease-9 inhibitors/metalloprotease-2 inhibitors, including but not limited to ERG-240;
BET inhibitors, including but not limited to GSK-3358699;
bispecific anti-CD86/IL-10, including but not limited to APVO-210;
bispecific humanized monoclonal antibody targeted against BAFF and IL-17A, including but not limited to tibulizumab;
bispecific antibody-peptide conjugate (BAFF/ICOSL), including but not limited to AMG-570;
B-lymphocyte antigen CD19 inhibitors, including but not limited to MDX-1342;
B-lymphocyte antigen CD19 inhibitors/immunoglobulin gamma Fc receptor IIB antagonists, including but not limited to XmAb-5871;
B-lymphocyte antigen CD20 inhibitors, including but not limited to ocrelizumab, ofatumumab, rituximab, ABP-798, Maball, Mabtas, Reditux, Zytux, veltuzumab, ocaratuzumab, BLX-301, IDEC-102, ABP-798, GP-2013, MK-8808, HLX-01, CT-P10, TL-011, PF-05280586, IBPM-001RX, IBI-301, AME-133v, BCD-020, BT-D004, SAIT-101, and JHL-1101;
B-lymphocyte antigen CD20 modulators, including but not limited to SBI-087, TRU-015, DXL-625, and Mabion CD20;
B-lymphocyte cell adhesion molecule inhibitor, including but not limited to SM-06;
B-lymphocyte stimulator ligand inhibitors, including but not limited to belimumab, RCT-18, blisibimod, tabalumab, and briobacept;
B-lymphocyte stimulator ligand/Tumor necrosis factor ligand 13 inhibitors, including but not limited to atacicept;
bradykinin receptor modulators/histone deacetylase inhibitors/P2X7 purinoceptor agonists, including but not limited to givinostat;
BRAF gene/MEK protein kinase/PERK gene inhibitors, including but not limited to binimetinib;
Bromodomain containing protein inhibitors, including but not limited to RVX-297, ZEN-003694;
Btk tyrosine kinase inhibitors, including but not limited to AC-0058, acalabrutinib, HM-71224, spebrutinib, BMS-986142, TAK-020, tirabrutinib (ONO-4059), TAS-5315, ABBV-105, GDC-0834, EBI-1459, BMS-986195, evobrutinib, fenebrutinib, SIMM-016, and YZJ-3058;
Btk tyrosine kinase inhibitors/Syk tyrosine kinase inhibitors/VEGF-2 receptor antagonists, including but not limited to CG-026806;
Btk tyrosine kinase inhibitors/IL-6 antagonists, including but not limited to RN-486;
Btk tyrosine kinase/Jak1 tyrosine kinase inhibitors, including but not limited to upadacitinib+ABBV-105;
Btk tyrosine kinase/Jak3 tyrosine kinase inhibitors, including but not limited to AC-0025;
cadherin-11 antagonists, including but not limited to RG-6125;
calcineurin inhibitors, including but not limited to ciclosporin;
calcineurin inhibitors/opioid receptor delta antagonists, including but not limited to HS-378;
calcium channel inhibitors, including but not limited to RP-3128;
calreticulin inhibitor, including but not limited to ALB-001 and ZYBK-2;
carbonic anhydrase/cyclooxygenase 2 inhibitors, including but not limited to polmacoxib;
cathepsin K inhibitors, including but not limited to CRA-013783 and VEL-0230;
cathepsin K/cathepsin S inhibitors, including but not limited to AM-3876 and NPI-2019;
cathepsin S inhibitors, including but not limited to MIV-247 and RWJ-445380;
CCR1 chemokine antagonists, including but not limited to BX-471, BMS-817399, BI-638683, CCX-354, MLN-3701, MLN-3897, CP-481715, and PS-375179;
CCR2 chemokine antagonists, including but not limited to MK-0812 and AZD-6942;
CCR3 gene modulators/eotaxin 2 ligand inhibitors, including but not limited to CM-102;
CCR5 chemokine antagonists, including but not limited to OHR-118, NIBR-6465, AZD-5672, and AZD-8566;
CD29 modulators/interleukin-10 ligands, including but not limited to PF-06687234;
CD3 modulators, including but not limited to otelixizumab;
CD39/CD73 agonists, including but not limited to AAV5-CD39/CD73 (rheumatoid arthritis), and Arthrogen;
CCR5 chemokine antagonists/CD4 agonists/HIV-1 gp120 protein inhibitors, including but not limited to maraviroc;
CD4 antagonists, including but not limited to zanolimumab, MTRX-1011A, BW-4162W94, EP-1645, clenoliximab, and DerG-PG275Cit;
CD40 ligand inhibitors, including but not limited to dapirolizumab pegol, and TNX-1500;
CD40 ligand receptor antagonists, including but not limited to BI-655064, anti-CD40-XTEN, teneliximab, VIB-4920, and iscalimab;
CD40 ligand receptor modulators/immunoglobulin G1 modulators, including but not limited to CFZ-533;
CD52 antagonists/clusterin stimulators, including but not limited to alemtuzumab;
bispecific CD32B/CD79B antibody, including but not limited to PRV-3279 (MGD-010);
CD80 antagonists, including but not limited to abatacept biobetter;
CD80 antagonists/T cell surface glycoprotein CD28 inhibitors, including but not limited to RhuDex;
CD80 antagonists/CD86 antagonists, including but not limited to XENP-9523 and ASP-2408;
CD86 antagonists, including but not limited to abatacept biosuperior;
CD86 antagonists/cytotoxic T-lymphocyte protein-4 modulators, including but not limited to ES-210;
CD95 antagonists, including but not limited to DE-098 and CS-9507;
Cell adhesion molecule inhibitors, including but not limited to alicaforsen, NPC-17923, TK-280, and PD-144795;
Chemokine receptor antagonists, including but not limited to PF-06835375;
Complement C5 factor inhibitors, including but not limited to eculizumab,
Complement C5 factor inhibitors/IL-1 antagonists, including but not limited to antisense oligonucleotides (rheumatoid arthritis) and Leiden University Medical Center Complement Factor stimulators, including but not limited to CM-101;
C-reactive protein inhibitors, including but not limited to ISIS-353512;
C-reactive protein inhibitors/cyclooxygenase 2 inhibitors/Nuclear factor kappa B inhibitors/immunoglobulin M antagonists/IL-2 receptor antagonists/PGE2 antagonists: IB-RACSF-1 antagonists, including but not limited to masitinib, FPA-008, JNJ-27301937, JNJ-40346527, PLX-5622, CT-1578, PD-360324, and JNJ-28312141;
CSF-1 antagonists/Fyn tyrosine kinase inhibitors/Kit tyrosine kinase inhibitors/Lyn tyrosine kinase inhibitors/NK cell receptor modulators/PDGF receptor antagonists, including but not limited to masitinib;
CXC10 chemokine ligand inhibitors, including but not limited to 946414-98-8 and BMS-936557;
CXCR4 chemokine antagonists, including but not limited to plerixafor;
CDK-2/7/9 inhibitors/MCL1 gene inhibitors, including but not limited to seliciclib;
CDK-1/2/5/7/9 inhibitors, including but not limited to BP-14;
Chaperonin modulator, including but not limited to IRL-201805;
cyclooxygenase 2 inhibitors, including but not limited to celecoxib, etoricoxib, meloxicam, and lumiracoxib;
cyclooxygenase 2/oxidoreductase inhibitors, including but not limited to etodolac;
cyclooxygenase 2 modulators, including but not limited to DRGT-46;
cyclooxygenase inhibitors, including but not limited to aceclofenac, diclofenac, naproxcinod, naproxen etemesil, nabumetone, Aleve, pelubiprofen, LY-210073, NS-398, bromfenac, L-746483, LY-255283, ibuprofen, flurbiprofen, SC-57666, and bermoprofen;
cyclooxygenase inhibitors/H+K+ ATPase inhibitors, including but not limited to naproxen+esomeprazole strontium;
cyclooxygenase inhibitors/PGE1 agonists, including but not limited to misoprostol+diclofenac;
cyclooxygenase inhibitors/oxidoreductase inhibitors, including but not limited to imidazole salicylate;
cytosolic phospholipase A2 inhibitors/phospholipase A2 inhibitors, including but not limited to AVX-002;
cytotoxic T-lymphocyte protein-4 stimulators/T cell surface glycoprotein CD28 inhibitors, including but not limited to abatacept, BMS-188667, and belatacept;
deoxyribonuclease gamma stimulator, including but not limited to NTR-441;
DHFR inhibitors, including but not limited to MPI-2505, Jylamvo, and ZeNEO-Methotrexate;
DHFR inhibitors/folate antagonists/transferrin modulators, including but not limited to methotrexate;
diamine acetyltransferase inhibitors, including but not limited to diminazene aceturate;
dihydroorotate dehydrogenase inhibitors, including but not limited to ASLAN-003, HWA-486, and ABR-224050;
dihydroorotate dehydrogenase/protein tyrosine kinase inhibitors, including but not limited to leflunomide;
DYRK-1 alpha protein kinase inhibitor, including but not limited to VRN-02;
elongation factor 2 inhibitors/interleukin-2 ligands/NAD ADP ribosyltransferase stimulators, including but not limited to denileukin diftitox;
enolase 1 inhibitor, including but not limited to HuL-001;
EP4 prostanoid receptor antagonists, including but not limited to CR-6086;
erythropoietin receptor agonists, including but not limited to cibinetide;
Fas ligands, including but not limited to AP-300;
FGF-2 ligand inhibitors, including but not limited to RBM-007;
FK506 binding protein-12 modulators/mTOR inhibitors, including but not limited to temsirolimus;
folate antagonists/transferrin modulators/DHFR inhibitors, including but not limited to MBP-Y003;
folate receptor modulators, including but not limited to technetium (99mTc) etarfolatide;
fractalkine ligand inhibitors, including but not limited to E-6011;
Fyn tyrosine kinase inhibitors/GABA A receptor modulators/cyclooxygenase 2 inhibitors/dihydroorotate dehydrogenase inhibitors, including but not limited to laflunimus;
glucocorticoid agonists, including but not limited to prednisone, prednisolone, and fosdagrocorat;
glucocorticoid antagonists, including but not limited to REC-200;
glucocorticoid induced leucine zipper stimulators, including but not limited to ART-GOT;
GM-CSF ligand inhibitors, including but not limited to namilumab, gimsilumab (MORAb-022), and TJM-2;
GM-CSF receptor antagonists, including but not limited to mavrilimumab;
GM-CSF receptor modulators, including but not limited to GSK-3196165 and otilimab;
growth regulated protein alpha ligand inhibitors/APT transcription factor inhibitors/IL-6 antagonists/interleukin-1 beta ligand inhibitors/cathepsin K inhibitors/NFAT gene inhibitors, including but not limited to T-5224;
H+K+ ATPase inhibitors, including but not limited to naproxen+esomeprazole, ketoprofen+omeprazole, KEO-25001, HC-1004, and PN-40020;
histamine H4 receptor antagonists, including but not limited to toreforant and GD-48;
histone deacetylase inhibitors, including but not limited to CHR-5154 (GSK-3117391) and NIPEP-CARE;
histone deacetylase-6 inhibitors, including but not limited to CKD-506;
HLA class II antigen DQ-2 alpha modulators, including but not limited to NexVax2;
HLA class II antigen inhibitors, including but not limited to HLA-DR1/DR4 inhibitors (rheumatoid arthritis) and Provid;
HLA class II antigen modulators, including but not limited to recombinant T-cell receptor ligand (rheumatoid arthritis) and Artielle;
Hsp 70 family inhibitors, including but not limited to gusperimus trihydrochloride;
hypoxia inducible factor-1 inhibitors/VEGF receptor antagonists, including but not limited to 2-methoxyestradiol;
IFNB gene stimulators, including but not limited to ART-102;
I-kappa B kinase beta inhibitors, including but not limited to IMD-2560;
I-kappa B kinase beta inhibitors/Nuclear factor kappa B inhibitors, including but not limited to IMD-0560;
I-kappa B kinase inhibitors/NFE2L2 gene stimulators/Nuclear factor kappa B inhibitors/STAT3 gene inhibitors, including but not limited to bardoxolone methyl;
IL-1 antagonists, including but not limited to recombinant human interleukin-1 receptor antagonist (rheumatoid arthritis), Shanghai Fudan-Zhangjiang Bio-Pharmaceutical;
IL-1 antagonists/interleukin-1 beta ligand inhibitors, including but not limited to rilonacept;
IL-10 agonists, including but not limited to peg-ilodecakin;
IL-11 agonists/PDGF receptor agonists, including but not limited to oprelvekin;
IL-12 antagonists/IL-23 antagonists, including but not limited to ustekinumab and briakinumab;
IL-15 antagonists, including but not limited to AMG-714;
IL-17 antagonists, including but not limited to ixekizumab and secukinumab;
IL-17 receptor modulators, including but not limited to CNTO-6785;
IL-2 receptor agonists, including but not limited to interleukin-2 follow-on biologic (IL-2), Anteluke, and Interking;
IL-2/IL-21/IL-15 antagonists, including but not limited to BNZ-132-2;
IL-21 antagonists, including but not limited to NN-8828;
IL-4 agonists, including but not limited to SER-130-AMI;
IL-6 antagonists, including but not limited to BCD-089, olokizumab, clazakizumab, sirukumab, SA-237, FB-704A, OP-R003, peptide IL-6 antagonist, MEDI-5117, AMG-220, FM-101, BLX-1025, esonarimod, TA-383, and sarilumab;
IL-6 antagonists/interleukin-1 beta ligand inhibitors/TNF alpha ligand inhibitors, including but not limited to K-832;
IL-6 antagonists/insulin sensitizers/interleukin-1 beta ligand inhibitors, including but not limited to BLX-1002;
IL-6 receptor antagonists/modulators, including but not limited to tocilizumab, HS-628, and LusiNEX;
IL-6 receptor modulators, including but not limited to BAT-1806 and RO-4877533;
immunoglobulin antagonists, including but not limited to iguratimod;
immunoglobulin G1 agonists, including but not limited to BX-2922 and HF-1020;
immunoglobulin G1 agonists/interleukin-1 beta ligand inhibitors, including but not limited to canakinumab;
immunoglobulin G1 agonists/TNF alpha ligand inhibitors, including but not limited to STI-002;
immunoglobulin G1 antagonists/TNF alpha ligand inhibitors, including but not limited to YHB-1411-2;
immunoglobulin G1 modulators/GM-CSF ligand inhibitors/immunoglobulin kappa modulators, including but not limited to lenzilumab;
immunoglobulin G2 antagonists/NF kappa B inhibitor stimulators/osteoclast differentiation factor antagonists/osteoclast differentiation factor ligand inhibitors/TNFSF 11 gene inhibitors, including but not limited to denosumab;
immunoglobulin gamma Fc receptor II modulators, including but not limited to MGD-010;
inducible nitric oxide synthase inhibitors/cyclooxygenase 2 inhibitors/MAP kinase modulators/nuclear factor kappa B inhibitors, including but not limited to SKLB-023;
inosine monophosphate dehydrogenase inhibitors, including but not limited to mizoribine;
insulin sensitizers/nuclear factor kappa B inhibitors/interleukin ligand inhibitors, including but not limited to HE-3286;
integrin alpha-1/beta-1 antagonists, including but not limited to SAN-300;
integrin alpha-4/beta-1 antagonists/cell adhesion molecule inhibitors, including but not limited to natalizumab;
integrin alpha-9 antagonist, including but not limited to ASP-5094;
integrin antagonists, including but not limited to PEG-HM-3 and CY-9652;
interferon beta ligands, including but not limited to recombinant interferon beta-1a;
interferon beta ligands/IL-6 antagonists, including but not limited to TA-383;
interferon gamma ligands, including but not limited to Li Zhu Yin De Fu and Clongamma;
interleukin 17A ligand inhibitors/tumor necrosis factor ligand inhibitors, including but not limited to ABT-122 and ABBV-257;
interleukin 17F ligand inhibitors, including but not limited to bimekizumab;
interleukin 18 ligand inhibitors, including but not limited to tadekinig alfa;
interleukin 23A inhibitors, including but not limited to guselkumab;
interleukin ligands/IL-1 antagonists, including but not limited to IBPB-007-IL;
interleukin receptor 17A antagonists, including but not limited to brodalumab;
interleukin-1 beta ligand inhibitors, including but not limited to gevokizumab, LY-2189102, CDP-484, and AR-100;
interleukin-1 beta ligand inhibitors/TNF alpha ligand inhibitors, including but not limited to PMI-001;
interleukin-1 beta ligands/TNF alphaligand modulators, including but not limited to PUR-0110;
interleukin-2 ligands, including but not limited to recombinant interleukin-2 and CUG-252;
IL-2 modulators, including but not limited to AMG-592;
interleukin-4 ligands/tenascin modulator, including but not limited to Tetravil;
interleukin-6 ligand inhibitors, including but not limited to gerilimzumab and PF-4236921;
IRAK-4 protein kinase inhibitor, including but not limited to BAY-1830839, BAY-1834845, PF-06650833, and KT-474;
Itk tyrosine kinase inhibitors, including but not limited to JTE-051;
Itk tyrosine kinase inhibitors/Jak3 tyrosine kinase inhibitors, including but not limited to ARN-4079;
JAK tyrosine kinase inhibitors, including but not limited to deuterated tofacitinib analog, SD-900, and WXSH-0150;
JAK tyrosine kinase inhibitors/Syk tyrosine kinase inhibitors, including but not limited to cerdulatinib and CVXL-0074;
Jak1 tyrosine kinase inhibitors, including but not limited to ABT-494 (upadacitinib), ruxolitinib, filgotinib, itacitinib, NIP-585, YJC-50018, GLPG-0555, MRK-12, and SHR-0302;
Jak1/3 tyrosine kinase inhibitors, including but not limited to tofacitinib, tofacitinib citrate, peficitinib, CKD-374, and CS-944X;
JAK 1/3 inhibitor/ROCK1/2 inhibitor: CPL-409116;
Jak1/2 tyrosine kinase inhibitors, including but not limited to baricitinib, ruxolitinib, LW-104, and TLL-018;
Jak2 tyrosine kinase inhibitors/CSF-1 antagonists, including but not limited to CT-1578;
JAK3 gene inhibitors, including but not limited to PF-06651600;
Jak3 tyrosine kinase inhibitors, including but not limited to decernotinib, DNX-04042, MTF-003, and PS-020613;
Jun N terminal kinase inhibitors, including but not limited to IQ-T S;
KCNA voltage-gated potassium channel-3 modulators, including but not limited to MRAD-P1;
Kelch like ECH associated protein 1 modulators/Nuclear erythroid 2-related factor 2 stimulators, including but not limited to dimethyl fumarate;
LanC like protein 2 modulators, including but not limited to BT-11 and BT-104;
LDL receptor related protein-1 stimulator, including but not limited to SP-16;
leukotriene BLT receptor antagonists/complement C5 factor inhibitors, including but not limited to nomacopan;
LITAF gene inhibitors/JAK3 gene inhibitors/MAP3K2 gene inhibitors/TNF antagonists, including but not limited to GBL-5b;
Lymphocyte function antigen-3 receptor antagonists, including but not limited to alefacept;
Macrophage mannose receptor 1 modulators, including but not limited to technetium Tc 99m tilmanocept;
MAdCAM inhibitors/immunoglobulin G2 modulators, including but not limited to PF-547659;
MAPKAPK5 inhibitors/matrix metalloprotease inhibitors, including but not limited to GLPG-0259;
MEK protein kinase inhibitors, including but not limited to AD-GL0001;
membrane copper amine oxidase inhibitors, including but not limited to BTT-1023, PRX-167700, and vepalimomab;
metalloprotease-9 inhibitors, including but not limited to GS-5745;
microbiome modulator, including but not limited to EDP-1815;
midkine ligand inhibitors, including but not limited to CAB-102;
mitochondrial 10 kDa heat shock protein stimulators, including but not limited to INV-103;
mTOR inhibitors, including but not limited to everolimus;
NAMPT gene inhibitors, including but not limited to ART-D01;
Nicotinic acetylcholine receptor antagonists, including but not limited to RPI-78 and RPI-MN;
NKG2 A B activating NK receptor antagonists, including but not limited to monalizumab;
NKG2 D activating NK receptor antagonists, including but not limited to NNC-0142-002;
nuclear factor kappa B inhibitors, including but not limited to dehydroxy methylepoxyquinomicin, MP-42, VGX-1027, SP-650003, MG-132, SIM-916, VGX-350, VGX-300, GIT-027, MLN-1145, and NVP-IKK-005;
nuclear factor kappa B modulators/nuclear factor kappa B p105 inhibitors/transcription factor RelB inhibitors/transcription factor p65 inhibitors, including but not limited to REM-1086;
osteoclast differentiation factor antagonists, including but not limited to cyclic peptidomimetics (rheumatoid arthritis/osteoporosis), University of Michigan;
p38 MAP kinase alpha inhibitors, including but not limited to VX-745, BMS-582949, and BMS-751324;
p38 MAP kinase inhibitors, including but not limited to BCT-197, losmapimod, and ARRY-797;
PDE 4 inhibitors, including but not limited to apremilast;
PDE 5 inhibitors, including but not limited to PDE5 inhibitors (rheumatoid arthritis), University of Rochester;
PDGF-B ligand inhibitors/VEGF receptor antagonists, including but not limited to SL-1026;
phosphoinositide-3 kinase delta inhibitors, including but not limited to CT-732, INK-007, and GNE-293;
phosphoinositide-3 kinase delta/gamma inhibitors, including but not limited to duvelisib and RP-6503;
phospholipase A2 inhibitors, including but not limited to AK-106, varespladib methyl, Ro-31-4493, BM-162353, Ro-23-9358, and YM-26734;
platelet activating factor receptor antagonists, including but not limited to piperidone hydrochloridum;
PPAR gamma agonists, including but not limited to rosiglitazone XR;
PPAR gamma agonists/insulin sensitizers, including but not limited to rosiglitazone;
programmed cell death protein 1 modulators, including but not limited to INSIX RA;
prostaglandin D synthase stimulators, including but not limited to HF-0220;
protein tyrosine kinase inhibitors, including but not limited to tairuimide;
PurH purine biosynthesis protein inhibitors/inosine monophosphate dehydrogenase inhibitors, including but not limited to mycophenolate mofetil;
Rev protein modulators, including but not limited to ABX-464;
RIP-1 kinase inhibitors, including but not limited to GSK-2982772 and VRN-04;
IL-17 antagonist/rho associated protein kinase 2 inhibitor, including but not limited to KD-025;
signal transducer CD24 modulators, including but not limited to CD24-IgFc;
sodium glucose transporter-2 inhibitors/PPAR gamma agonists/insulin sensitizers, including but not limited to THR-0921;
STAT3 gene inhibitors, including but not limited to vidofludimus;
STAT-3 inhibitors, including but not limited to HL-237;
Superoxide dismutase stimulators, including but not limited to imisopasem manganese;
SYK family tyrosine kinase inhibitors/Zap70 tyrosine kinase inhibitors, including but not limited to MK-8457;
Syk tyrosine kinase inhibitors, including but not limited to fostamatinib, entospletinib, KDDF-201110-06, HMPL-523, AB-8779, GS-9876, PRT-2607, CG-103065, and SKI-O-703;
T cell receptor antagonists, including but not limited to TCR inhibiting SCHOOL peptides (systemic/topical, rheumatoid arthritis/dermatitis/scleroderma), SignaBlok and CII modified peptide (rheumatoid arthritis);
T cell receptor modulators/HLA class II antigen modulators, including but not limited to ARG-301;
T cell surface glycoprotein CD28 stimulators, including but not limited to TAB-08 and theralizumab;
TAK1 binding protein modulators, including but not limited to epigallocatechin 3-gallate;
Talin modulators, including but not limited to short-formtalin regulators (rheumatoid arthritis), KayteeBio;
T-cell differentiation antigen CD6 inhibitors, including but not limited to itolizumab;
T-cell surface glycoprotein CD8 inhibitors/TGF beta agonists/CD4 antagonists, including but not limited to tregalizumab;
thymulin agonists, including but not limited to Syn-1002;
TLR-2/TLR-4 antagonists, including but not limited to VB-201;
TLR-4 antagonists, including but not limited to NI-0101;
TLR-2/4/9 antagonists, including but not limited to P-13;
TNF agonists/TNF antagonists/type II TNF receptor modulators, including but not limited to Lifmior;
TNF alpha ligand inhibitors, including but not limited to Adfrar, FKB-327, Exemptia, Cinnora, Mabura, adalimumab, infliximab, Flixabi, PF-06438179, hadlima, recombinant humanized anti-TNF-alpha monoclonal antibody, CMAB-008, CT-P13, GB-242, golimumab (CNTO-148), ozoralizumab, AT-132, ISIS-104838, ISU-202, CT-P17, MB-612, Debio-0512, anti-TNF alpha human monoclonal antibody, UB-721, KN-002, DA-3113, BX-2922, R-TPR-015, BOW-050, PF-06410293, CKD-760, CHS-1420, GS-071, ABP-710, BOW-015, HLX-03, BI-695501, MYL-1401A, ABP-501, BAX-2923, SCH-215596, ABT-D2E7, BAT-1406, XPro-1595, Atsttrin, SSS-07, golimumabbiosimilar, TA-101, BLX-1002, ABX-0401, TAQ-588, TeHL-1, placulumab, CYT-007-TNFQb, SSR-150106, PassTNF, Verigen, DOM-0200, DOM-0215, AME-527, anti-TNF-alpha mAb, GENZ-38167, BLX-1028, CYT-020-TNFQb, CC-1080, CC-1069, LBAL, GP-2017, Idacio, IBI-303, HS-016, TNF-2, and IA-14069;
TNF alpha ligand inhibitors/TNF antagonists/type II TNF receptor modulators, including but not limited to BAX-2200;
TNF alpha ligand inhibitors/Type II TNF receptor modulators, including but not limited to Eucept, TNF alphaligand modulators: MM-A01-01, CDP-571, camobucol, and JNJ-63823539;
TNF antagonists, including but not limited to DNX-114, TNF antagonist+IL-12 antagonist (rheumatoid arthritis), University of Oxford, BN-006, pegsunercept, ACE-772, onercept, DE-096, PN-0615, lenercept, ITF-1779, MDL-201112, HD-203, Qiangke, and TNF a Fc;
TNF antagonists/type II TNF receptor modulators, including but not limited to Altebrel, Intacept, QL-0902, etanercept, Erelzi, opinercept, YISAIPU, Anbainuo, Benepali, YLB-113, SCB-808, DA-3853, and SCB-131;
TNF antagonists/TNF alpha ligand inhibitors, including but not limited to certolizumab pegol;
TNF receptor modulators, including but not limited to recombinant TNF receptor 2-Fc fusion protein mutant, T-0001;
TNF receptor modulators/TNF alpha ligand inhibitors, including but not limited to tgAAV-TNFR:Fc;
tumor necrosis factor 13C receptor antagonists, including but not limited to VAY-736;
tumor necrosis factor 15 ligand inhibitors, including but not limited to anti-TL1A antibodies (rheumatoid arthritis/inflammatory bowel disease), NIAMS;
tumor necrosis factor ligand inhibitors, including but not limited to etanercept biosimilar;
type I IL-1 receptor antagonists, including but not limited to anakinra, IL-1 R^{1a}, anakinra follow-on biologic, and AXXO;
type I TNF receptor antagonists, including but not limited to NM-940 and EN-2001;
type II TNF receptor modulators, including but not limited to LBEC-0101, DMB-3853, DWP-422, and BT-D001;
unspecified GPCR agonists, including but not limited to NCP-70X;
VEGF receptor antagonists, including but not limited to NSC-650853;
VEGF-2 receptor modulators, including but not limited to VEGFR2 neutralizing antibody (rheumatoid arthritis), University of Rochester;
VEGF-B ligand inhibitors, including but not limited to CSL-346;
X-linked inhibitor of apoptosis protein inhibitors, including but not limited to IAP inhibitors (oral), Pharmascience; and
Zap70 tyrosine kinase inhibitors, including but not limited to CT-5332.

### Inflammatory Bowel Disease Combination Therapy

In some embodiments, the compounds provided herein, or pharmaceutically acceptable salts thereof, may be combined with one or more additional therapeutic agents that treat or ameliorate inflammatory bowel disease (IBD).

The term “inflammatory bowel disease” or “IBD” as used herein is a collective term describing inflammatory disorders of the gastrointestinal tract, the most common forms of which are ulcerative colitis and Crohn's disease. Other forms of IBD that can be treated with the compounds provided herein, or pharmaceutically acceptable salts thereof, or pharmaceutical compositions provided herein include, but are not limited to, diversion colitis, ischemic colitis, infectious colitis, chemical colitis, microscopic colitis (including collagenous colitis and lymphocytic colitis), atypical colitis, pseudomembranous colitis, fulminant colitis, autistic enterocolitis, indeterminate colitis, Behçet's disease, gastroduodenal CD, jejunoileitis, ileitis, ileocolitis, Crohn's (granulomatous) colitis, irritable bowel syndrome, mucositis, radiation induced enteritis, short bowel syndrome, celiac disease, stomach ulcers, diverticulitis, pouchitis, proctitis, and chronic diarrhea.

Treating or preventing IBD also includes ameliorating or reducing one or more symptoms of IBD. As used herein, the term “symptoms of IBD” refers to detected symptoms such as abdominal pain, diarrhea, rectal bleeding, weight loss, fever, loss of appetite, and other more serious complications, such as dehydration, anemia and malnutrition. A number of such symptoms are subject to quantitative analysis (e.g., weight loss, fever, anemia, etc.). Some symptoms are readily determined from a blood test (e.g., anemia) or a test that detects the presence of blood (e.g., rectal bleeding). The term “wherein said symptoms are reduced” refers to a qualitative or quantitative reduction in detectable symptoms, including but not limited to a detectable impact on the rate of recovery from disease (e.g., rate of weight gain). The diagnosis is typically determined by way of an endoscopic observation of the mucosa, and pathologic examination of endoscopic biopsy specimens.

The course of IBD varies and is often associated with intermittent periods of disease remission and disease exacerbation. Various methods have been described for characterizing disease activity and severity of IBD as well as response to treatment in subjects having IBD. Treatment according to the present methods and uses is generally applicable to a subject having IBD of any level or degree of disease activity.

The methods and uses provided herein can also be applied at any point in the course of the disease. In some embodiments, the methods and uses are applied to a subject having IBD during a time period of remission (i.e., inactive disease). In some embodiments, the present methods and uses provided herein provide benefit by extending the time period of remission (e.g., extending the period of inactive disease) or by preventing, reducing, or delaying the onset of active disease. In some embodiments, the methods and uses provided herein may be applied to a subject having IBD during a period of active disease. In some embodiments, the methods and uses provided herein provide benefit by reducing the duration of the period of active disease, reducing or ameliorating one or more symptoms of IBD, or treating IBD.

Measures for determining efficacy of treatment of IBD in clinical practice have been described and include, for example, the following: symptom control; fistula closure; extent of corticosteroid therapy required; and improvement in quality of life. Heath-related quality of life (HRQL) can be assessed using the Inflammatory Bowel Disease Questionnaire (IBDQ), which is extensively used in clinical practice to assess quality of life in a subject with IBD. (See Guyatt et al. (1989) Gastroenterology 96:804-810.) In some embodiments, the compounds provided herein, or pharmaceutically acceptable salts thereof, may be combined with one or more additional therapeutic agents that treat or ameliorate IBD.

Non-limiting examples of therapeutic agents that treat or ameliorate IBC include allogeneic bone marrow-derived MSC therapy, AMP activated protein kinase stimulator, aryl hydrocarbon receptor agonist and T cell receptor modulator, ASK1 inhibitors, beta adrenoceptor antagonists, BTK inhibitors, beta-catenin stimulator, beta-glucuronidase inhibitors, bradykinin receptor modulators, calcineurin inhibitors, calcium channel inhibitors, cathepsin S inhibitors, CCR3 chemokine antagonists, CD40 ligand receptor antagonists, chemokine CXC ligand inhibitors, CHST15 gene inhibitors, collagen modulators, CXCR3 chemokine antagonist, CSF-1 antagonists, cyclooxygenase inhibitors, cytochrome P450 3A4 inhibitors, DYRK-1 alpha protein kinase inhibitor, endothelial dysfunction and vascular leakage blocker, enolase 1 inhibitor, eotaxin ligand inhibitors, EP4 prostanoid receptor agonists, erythropoietin receptor agonists, exportin 1 inhibitor, fractalkine ligand inhibitors, free fatty acid receptor2 antagonists, GATA 3 transcription factor inhibitors, glucagon-like peptide 2 agonists, glucocorticoid agonists, guanylate cyclase receptor agonists, histone deacetylase inhibitors, HLA class II antigen modulators, IL-12 antagonists, IL-13 antagonists, Interleukin-2 ligand, IL-23 antagonists, IL-6 antagonists, IL-6 receptor modulators, interleukin-7 receptor modulators, IL-7 antagonists, IL-8 antagonists, integrin alpha-4/beta-1 antagonists, integrin alpha-4/beta-7 antagonists, integrin alpha-E antagonists, integrin antagonists, integrin beta-7 antagonists, interleukin ligand inhibitors, Interleukin-10 ligand, interleukin receptor 17A antagonists, Interleukin 23A inhibitor, interleukin-1 beta ligands, interleukin-1 beta ligand modulators, IRAK4 inhibitors, JAK tyrosine kinase inhibitors, Jak1 tyrosine kinase inhibitors, Jak3 tyrosine kinase inhibitors, LanC like protein 2 modulators, lipoxygenase modulators, acrophage mannose receptor 1 modulator, MAdCAM inhibitors, matrix metalloprotease inhibitors, melanocortin agonists, metalloprotease-9 inhibitors, NADPH oxidase inhibitor, natriuretic peptide receptor C agonists, NC-301, next-generation intestinal microbiota therapy, neuregulin-4 ligands, NKG2 D activating NK receptor antagonists, Non receptor tyrosine kinase TYK2 antagonist, opioid receptor antagonists, opioid receptor delta antagonists, oxidoreductase inhibitors, P2X7 purinoceptor agonists, PDE 4 inhibitors, phagocytosis stimulating peptide modulators, potassium channel inhibitors, PPAR alpha agonists, PPAR delta agonists, PPAR gamma agonists, protein fimH inhibitors, P-selectin glycoprotein ligand-1 inhibitors, RNA polymerase inhibitors, sphingosine 1 phosphate phosphatase 1 stimulators, sphingosine 1 phosphate phosphatase modulators, sphingosine-1-phosphate receptor-1 agonists, sphingosine-1-phosphate receptor-1 antagonists, sphingosine-1-phosphate receptor-1 modulators, sphingosine-1-phosphate receptor-5 modulators, STAT3 gene inhibitors, stem cell antigen-1 inhibitors, superoxide dismutase modulators, superoxide dismutase stimulators, SYK inhibitors, TGF beta 1 ligand inhibitors, thymulin agonists, TLR antagonists, TNF alphaligand inhibitors, TNF antagonists, tumor necrosis factor 14 ligand modulators, type II TNF receptor modulators, Tpl 2 inhibitors, X box binding protein 1 stimulator, and Zonulin inhibitors.

In some embodiments, the compounds provided herein, or pharmaceutically acceptable salts thereof, may be combined with one or more additional therapeutic agents selected from ABX-464, adalimumab; ALLO-ASC-CD, AMG-966, AMT-101, anakinra, apremilast; Alequel; ALV-304, AMG-139; amiselimod, anti-CXCR3 mAb, ASD-003, ASP-3291, AX-1505, balsalazide; beclomethasone dipropionate; BI-655130, BMC-321, BMC-322, BMS-986184; BT-051, budesonide; CBX-111, CEQ-508; certolizumab; cibinetide, Clostridium butyricum; ChAdOx2-HAV, CU-06, CUG-252 dexamethasone sodium phosphate, DNVX-078, EB-7020, EM-101, etanercept; ENERGI-F704, ETX-201, golimumab; GS-4997, GS-5718, GS-9876, GS-4875, GS-4059, infliximab; IMS-001, mesalazine, HLD-400, IBI-112, IMM-H013, KB-295, LFS-829, LYC-30937 EC; IONIS-JBI1-2.5Rx, JNJ-64304500, JNJ-66525433, JNJ-4447, mesalamine, MET-642, MVA-HAV, naltrexone; natalizumab; neihulizumab, olsalazine; NOS-1244, NTG-A-009, PH-46-A, propionyl-L-carnitine; PTG-100; remestemcel-L; tacrolimus; teduglutide; tofacitinib; ASP-1002; ustekinumab; vedolizumab; AVX-470; INN-108; SGM-1019; PF-06480605; PF-06651600; PR-600; RBX-8225, R-2187, RG-6287, SER-287; TOP-1288; VBY-129; 99mTc-annexin V-128; bertilimumab; DLX-105; dolcanatide; quetmolimab (E-6011); FFP-104; filgotinib; foralumab; GED-0507-34-Levo; givinostat; GLPG-0974; iberogast; ICP-330, JNJ-40346527; K(D)PT; KAG-308; KHK-4083; KRP-203; larazotide acetate; LY-3074828, midismase; olokizumab; OvaSave; P-28-GST; PF-547659; prednisolone; QBECO; RG-7835; RBX-2660, RO7049665, JKB-122; SYGN-313, SB-012; STNM-01; SZN-1326, TJC-0434, Debio-0512; TRK-170; ABT-494; Ampion; BI-655066; carotegast methyl; cobitolimod; elafibranor; etrolizumab; GS-5745; HMPL-004; LP-02, ozanimod; peficitinib; QX-004-N, RHB-104; SEFA-1024, tildrakizumab; TOP-1890, tralokinumab; brodalumab; laquinimod; and plecanatide; or a pharmaceutically acceptable salt of any of the foregoing; or any combination thereof.

### NUMBERED EMBODIMENTS

Clause 1. A compound of Formula (I)

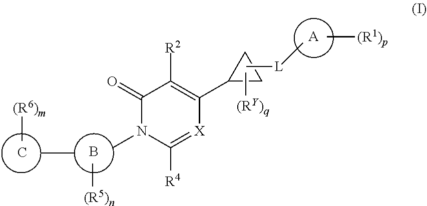

or a pharmaceutically acceptable salt thereof, wherein ring \textcircled{A} is C₆₋₁₀ aryl, 5- to 10-membered heteroaryl, C₃₋₁₀ cycloalkyl, or 4- to 10-membered heterocyclyl;
each R¹ is independently halogen, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g};
each R^{1a} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, two R^{1a} groups taken together with the nitrogen atom to which they are attached form a 4- to 10-membered heterocyclyl, wherein each alkyl, alkenyl, alkynyl, cycloalkyl, heterocyclyl, aryl, and heteroaryl is optionally substituted with one to three R¹ⁱ;
each R^{1g} is independently —CN, C₁₋₆ alkyl, C₂₋₆ alkynyl, C₃₋₁₀ cycloalkyl, 4- to 10-membered heterocyclyl, C₆₋₁₀ aryl, 5- to 10-membered heteroaryl, —OR^{1h}, —N(R^{1h})(R^{1h}), —C(O)N(R^{1h})(R^{1h}), —N(R^{1h})C(O-R^{1h}, —N(R^{1h})C(O)O—R^{1h}, —N(R^{1h})C(O)N(R^{1h})(R^{1h}), —OC(O)N(R^{1h})(R^{1h}), —N(R^{1a})S(O)₂(R^{1a}), —S(O)₂R^{1h}, or —S(O)₂N(R^{1a})(R^{1a}), wherein each alkyl, alkynyl, cycloalkyl, heterocyclyl, aryl, and heteroaryl is optionally substituted with one to three R¹ⁱ;
each R^{1h} is independently H, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, or two R^{1h} groups taken together with the nitrogen atom to which they are attached form a 4- to 10-membered heterocyclyl, wherein each alkyl, alkynyl, cycloalkyl, heterocyclyl, aryl and heteroaryl may be further substituted with one to three R¹ⁱ;
each R¹ⁱ is independently halogen, —OH, —NH₂, —CN, C₁₋₆ alkyl, C₁₋₆ haloalkyl, C₁₋₆ hydroxyalkyl, C₃₋₆ cycloalkyl, C₃₋₆ halocycloalkyl, C₁₋₆ alkoxy, C₁₋₆ haloalkoxy, or —O(C₃₋₆ cycloalkyl); L is a bond, —O—, —NR^{La}—, —C(O)NR^{La}—, —C(O)—, or C₁₋₃ alkylene, wherein the alkylene is optionally substituted with one to three R^{Lb};
each R^{La} is independently H, C₁₋₆ alkyl, C₁₋₆ haloalkyl, or C₃₋₁₀ cycloalkyl;
each R^{Lb} is independently halogen, —OH, —OCH₃, —NH₂, or —CN;
each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy;
or R^{Y} and R¹ join together to form a C₃₋₆ cycloalkyl or 4- to 6-membered heterocyclyl, wherein each cycloalkyl and heterocyclyl is optionally substituted with one to three halogen;
X is CR³ or N;
R² is H, halogen, C₁₋₆ alkyl, or C₃₋₆ cycloalkyl, wherein each alkyl and cycloalkyl is optionally substituted with one to three halogen;
R³ is H, halogen, —CN, C₁₋₆ alkyl, C₁₋₆ haloalkyl, C₁₋₆ hydroxyalkyl, C₁₋₆ alkoxy, C₁₋₆ haloalkoxy, or —O(C₃₋₆ cycloalkyl);
R⁴ is H, —CN, C₁₋₆ alkyl, C₂₋₆ alkynyl, or C₃₋₁₀ cycloalkyl, wherein each alkyl, alkynyl, and cycloalkyl is optionally substituted with one to three R^{4a1};
each R^{4a1} is independently halogen, —CN, —OR^{4a2}, or —NR^{4a2}R^{4a2};
each R^{4a2} is independently H, C₁₋₆ alkyl, C₁₋₆ haloalkyl, C₃₋₆ cycloalkyl, —C(O)C₁₋₆ alkyl, —C(O)O(C₁₋₆ alkyl), or —C(O)N(R^{4a3})₂;
each R^{4a3} is independently C₁₋₆ alkyl;
ring \textcircled{B} is C₆₋₁₀ aryl or 5- to 10-membered heteroaryl;
each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one to three R^{5b1};
each R^{5a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with one to three R^{5b3};
each R^{5b1} is independently halogen, —CN, C₁₋₆ alkyl, C₂₋₆ alkynyl, C₁₋₆ haloalkyl, C₃₋₁₀ cycloalkyl, 4- to 10-membered heterocyclyl, —OR¹², or —N(R^{5b2})₂, wherein each alkyl, alkynyl, haloalkyl, cycloalkyl, and heterocycle is optionally substituted with one to three R^{5b3};
each R^{5b2} is independently H, C₁₋₆ alkyl, or C₃₋₁₀ cycloalkyl, wherein each alkyl and cycloalkyl is optionally substituted with one or two oxo and is further optionally substituted with one to three R^{5b3};
each R^{5b3} is independently halogen, —CN, —OH, —NH₂, C₁₋₆ alkyl, C₁₋₆ haloalkyl, C₁₋₆ hydroxyalkyl, C₁₋₆ alkoxy, C₁₋₆ haloalkoxy, C₃₋₆ cycloalkyl, C₃₋₆ halocycloalkyl, or —O(C₃₋₆ cycloalkyl); ring \textcircled{C} is C₆₋₁₀ aryl, 5- to 10-membered heteroaryl, C₃₋₈ cycloalkyl, or 4- to 10-membered heterocyclyl;
each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further optionally substituted with one to three R^{6b1};
each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R⁶¹ groups taken together with the atom or atoms to which they are attached form a 4- to 10-membered heterocyclyl, wherein each alkyl, alkenyl, cycloalkyl, heterocyclyl, aryl, and heteroaryl is optionally substituted with one to three R^{6b1};
each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is optionally substituted with one to three R^{6c1};
each R^{6b2} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, wherein each alkyl, alkenyl, cycloalkyl, heterocyclyl, aryl, and heteroaryl is optionally substituted with one to three R^{6c2};
each R^{6c1} and R^{6c2} is independently oxo, halogen, —NO₂, —N₃, —CN, —OH, R^{6d1}, —O(R^{6d1}), —OC(O)(R^{6d1}), —NH₂, —NH(R^{6d1}), —N(R^{6d1})₂, —C(O)(R^{6d1}), —C(O)O(R^{6d1}), —C(O)NH₂, —C(O)NH(R^{6d1}), —C(O)N(R^{6d1})₂, —NHC(O)(R^{6d1}), —NHC(O)O(R^{6d1}), —NHC(O)NH(R^{6d1}), —SH, —S(R^{6d1}), —NHS(O)(R^{6d1}), —N(R^{6d1})(S(O)(R^{6d1}), —S(O)N(R^{6d1})₂, —S(O)(R^{6d1}), —S(O)(NH)(R^{6d1}), —S(O)₂(R^{6d1}), —S(O)₂NH(R^{6d1}), or —S(O)₂N(R^{6d1})₂;
each R^{6d1} is independently C₁₋₆ alkyl, C₃₋₁₀ cycloalkyl, 4- to 10-membered heterocyclyl, C₆₋₁₀ aryl, or 5- to 10-membered heteroaryl, wherein each alkyl, alkenyl, alkynyl, cycloalkyl, heterocyclyl aryl, and heteroaryl is optionally substituted with one to three R^{6e1};
each R^{6e1} is independently oxo, halogen, —NO₂, —N₃, —CN, —OH, —NH₂, methyl, or halomethyl;
wherein each heterocyclyl and heteroaryl independently has one to four ring heteroatoms each independently selected from N, O, and S; and
   each m, n, p and q is independently 0, 1, 2 or 3.

Clause 2. The compound of clause 1 having Formula (II)

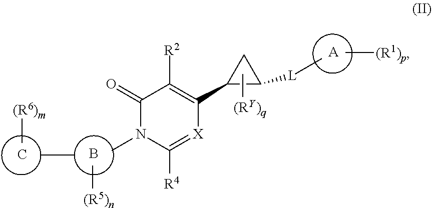

or a pharmaceutically acceptable salt thereof.
Clause 3. The compound of clause 1 or 2, or a pharmaceutically acceptable salt thereof, wherein \textcircled{A} is C₆₋₁₀ aryl or 5- to 10-membered heteroaryl.

Clause 4. The compound of clause 1 or 2, or a pharmaceutically acceptable salt thereof, wherein \textcircled{A} is phenyl.

Clause 5. The compound of clause 1 or 2, or a pharmaceutically acceptable salt thereof, wherein \textcircled{A} is 5- to 9-membered heteroaryl.

Clause 6. The compound of clause 1 or 2, or a pharmaceutically acceptable salt thereof, wherein \textcircled{A} is 5-membered heteroaryl.

Clause 7. The compound of clause 1 or 2, or a pharmaceutically acceptable salt thereof, wherein \textcircled{A} is thiazolyl, isothiazolyl, pyrazolyl, imidazolyl, or pyrrolyl.

Clause 8. The compound of clause 1 or 2, or a pharmaceutically acceptable salt thereof, wherein \textcircled{A} is 6-membered heteroaryl.

Clause 9. The compound of clause 1 or 2, or a pharmaceutically acceptable salt thereof, wherein \textcircled{A} is pyridinyl, pyrimidinyl, pyrazinyl, or pyridazinyl.

Clause 10. The compound of clause 1 or 2, or a pharmaceutically acceptable salt thereof, wherein ring \textcircled{A} is

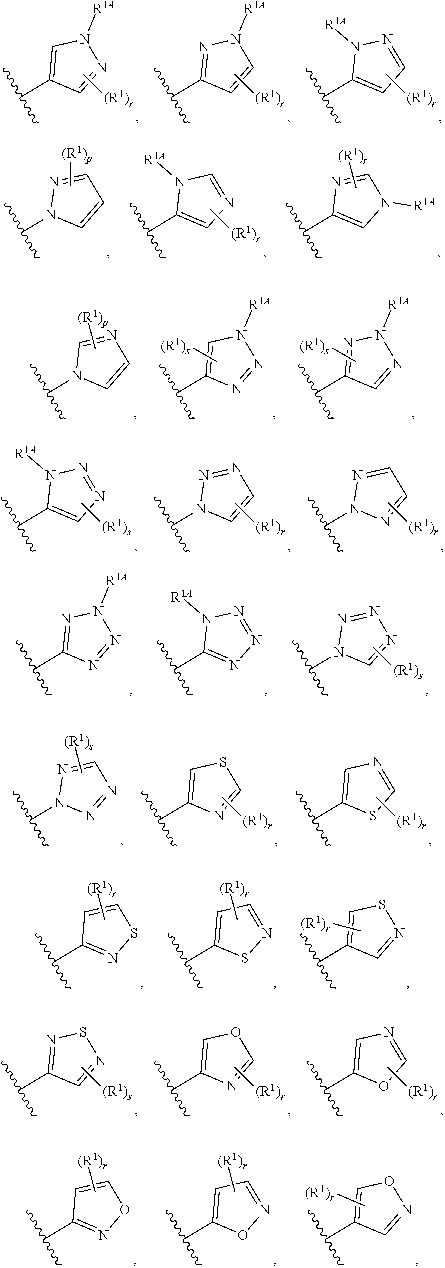

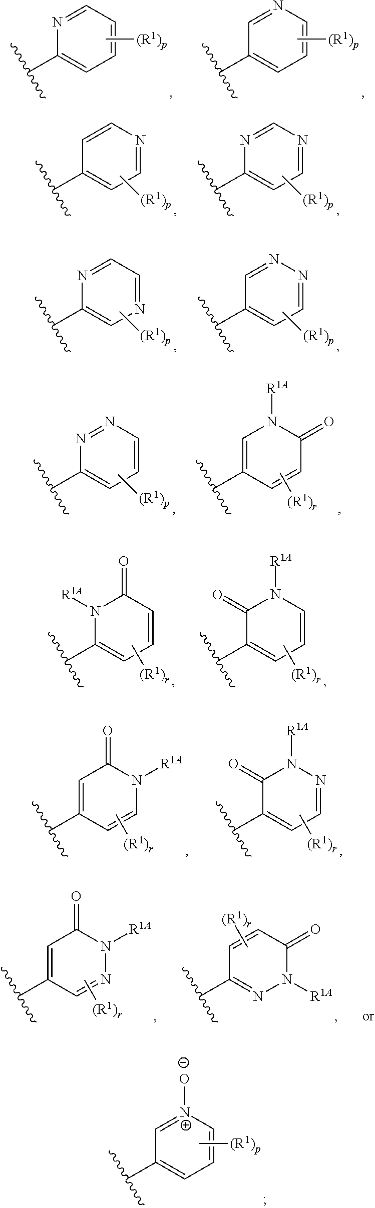

R^{1A} is H or R¹;
r is 0, 1, or 2; and
s is 0 or 1.

Clause 11. The compound of clause 1 or 2, or a pharmaceutically acceptable salt thereof, wherein ring \textcircled{A} is

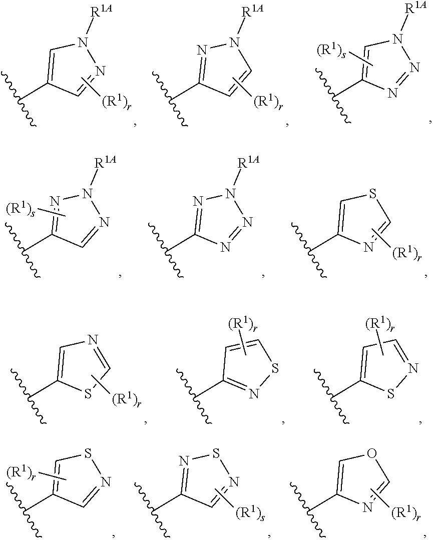

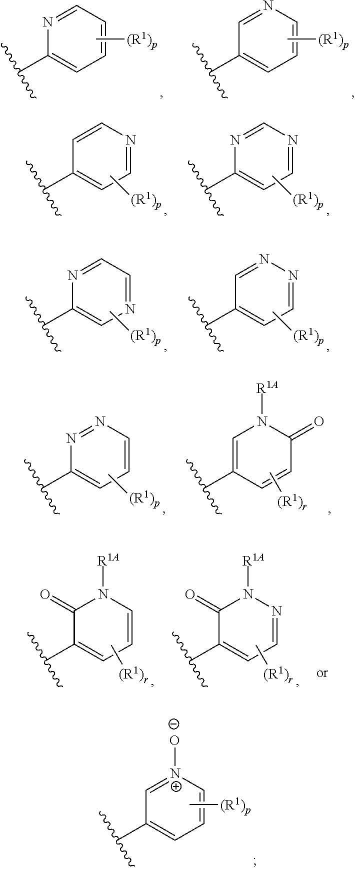

R^{1A} is H or R¹;
r is 0, 1, or 2; and
s is 0 or 1.

Clause 12. The compound of clause 1 or 2, or a pharmaceutically acceptable salt thereof, wherein ring \textcircled{A} is

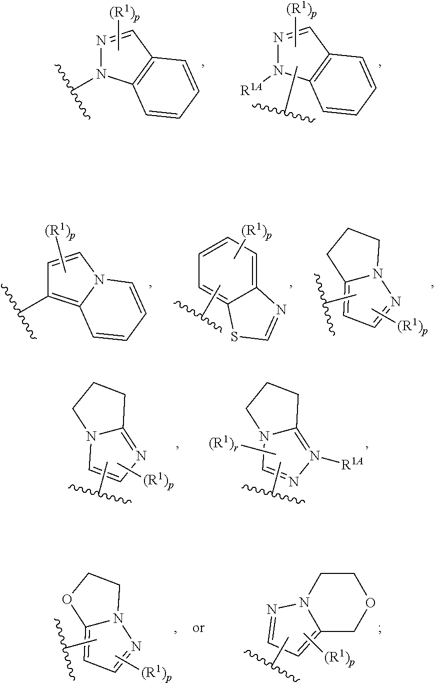

R^{1A} is H or R¹; and
r is 0, 1, or 2.

Clause 13. The compound of clause 1 or 2, or a pharmaceutically acceptable salt thereof, wherein ring \textcircled{A} is

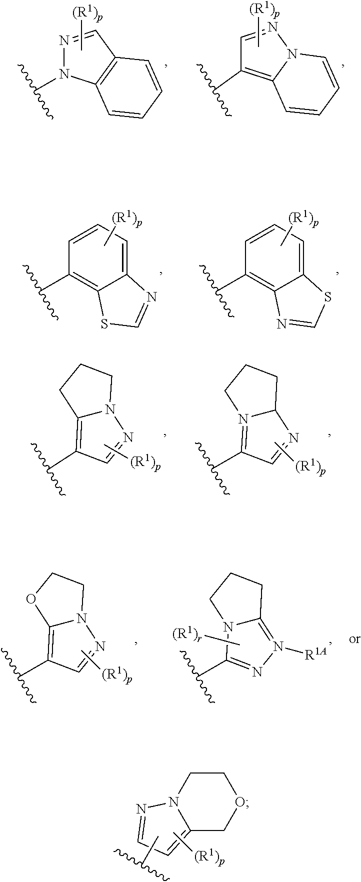

R^{1A} is H or R¹; and
r is 0, 1, or 2.

Clause 14. The compound of clause 1 or 2, or a pharmaceutically acceptable salt thereof, wherein ring \textcircled{A} is

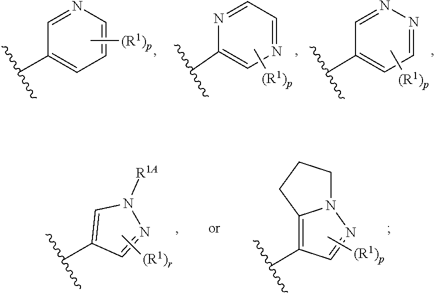

Clause 15. The compound of clause 1 or 2, or a pharmaceutically acceptable salt thereof, wherein ring \textcircled{A} is

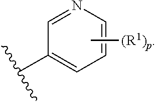

Clause 16. The compound of clause 1 or 2, or a pharmaceutically acceptable salt thereof, wherein ring \textcircled{A} is

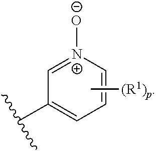

Clause 17. The compound of clause 1 or 2, or a pharmaceutically acceptable salt thereof, wherein ring \textcircled{A} is

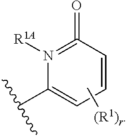

R^{1A} is H or R¹; and
r is 0, 1, or 2.

Clause 18. The compound of clause 1 or 2, or a pharmaceutically acceptable salt thereof, wherein ring \textcircled{A} is

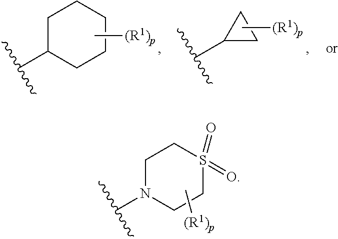

Clause 19. The compound of any one of clauses 1 to 18, or a pharmaceutically acceptable salt thereof, wherein p is 0, 1, or 2.

Clause 20. The compound of clause 19, or a pharmaceutically acceptable salt thereof, wherein p is 1 or 2.

Clause 21. The compound of any one of clauses 1 to 20, or a pharmaceutically acceptable salt thereof, wherein each R¹ is independently halogen, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g}.

Clause 22. The compound of any one of clauses 1 to 21, or a pharmaceutically acceptable salt thereof, wherein two R¹ groups taken together with two adjacent atoms of ring \textcircled{A} to which they are attached forma 4- to 6-membered heterocyclyl, wherein the heterocyclyl is optionally substituted with one to three halogen and is further optionally substituted with one to three R^{1g}.

Clause 23. The compound of any one of clauses 1 to 22, or a pharmaceutically acceptable salt thereof, wherein two R¹ groups taken together with two adjacent atoms of ring \textcircled{A} to which they are attached forma 5- to 6-membered heterocyclyl, wherein the heterocyclyl is optionally substituted with one to three halogen and is further optionally substituted with one to three R^{1g}.

Clause 24. The compound of any one of clauses 1 to 23, or a pharmaceutically acceptable salt thereof, wherein R^{1g} is C₁₋₆ alkyl, wherein each alkyl is optionally substituted with one to three halogen.

Clause 25. The compound of clause 24, or a pharmaceutically acceptable salt thereof, wherein R^{1g} is C₁₋₃ alkyl.

Clause 26. The compound of any one of clauses 1 to 23, or a pharmaceutically acceptable salt thereof, wherein the heterocyclyl is not substituted with R^{1g}.

Clause 27. The compound of any one of clauses 1 to 20, or a pharmaceutically acceptable salt thereof, wherein each R¹ is independently halogen, C₁₋₆ alkyl, C₃₋₁₀ cycloalkyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —N═S(O)(R^{1a})(R^{1a}) or two R¹ groups taken together with two adjacent atoms of ring \textcircled{A} to which they are attached form a 4- to 10-membered heterocyclyl, wherein each alkyl, cycloalkyl, and heterocyclyl is optionally substituted with one to three halogen.

Clause 28. The compound of clause 27, or a pharmaceutically acceptable salt thereof, wherein each R¹ is independently halogen, C₁₋₆ alkyl, or —C(O)N(R^{1a})(R^{1a}), wherein each alkyl is optionally substituted with one to three halogen.

Clause 29. The compound of any one of clauses 1 to 28, or a pharmaceutically acceptable salt thereof, wherein each R^{1a} is independently H, C₁₋₆ alkyl, or C₃₋₁₀ cycloalkyl wherein each alkyl and cycloalkyl is optionally substituted with one to three R¹ⁱ.

Clause 30. The compound of any one of clauses 1 to 29, or a pharmaceutically acceptable salt thereof, wherein each R¹ⁱ is halogen.

Clause 31. The compound of any one of clauses 1 to 29, or a pharmaceutically acceptable salt thereof, wherein each R^{1a} is independently H, C₁₋₃ alkyl, or C₃₋₆ cycloalkyl.

Clause 32. The compound of any one of clauses 1 to 20, or a pharmaceutically acceptable salt thereof, wherein each R¹ is independently halogen or C₁₋₃ alkyl.

Clause 33. The compound of any one of clauses 1 to 32, or a pharmaceutically acceptable salt thereof, wherein at least one R¹ is chloro.

Clause 34. The compound of any one of clauses 1 to 33, or a pharmaceutically acceptable salt thereof, wherein at least one R¹ is fluoro.

Clause 35. The compound of any one of clauses 1 to 34, or a pharmaceutically acceptable salt thereof, wherein q is 1.

Clause 36. The compound of any one of clauses 1 to 35, or a pharmaceutically acceptable salt thereof, wherein R^{Y} is halogen.

Clause 37. The compound of any one of clauses 1 to 34, or a pharmaceutically acceptable salt thereof, wherein q is 0.

Clause 38. The compound of any one of clauses 1 to 37, or a pharmaceutically acceptable salt thereof, wherein L is —C(O)NR^{La}— or —C(O)—.

Clause 39. The compound of any one of clauses 1 to 38, or a pharmaceutically acceptable salt thereof, wherein R^{La} is H.

Clause 40. The compound of any one of clauses 1 to 38, or a pharmaceutically acceptable salt thereof, wherein R^{La} is methyl.

Clause 41. The compound of any one of clauses 1 to 37, or a pharmaceutically acceptable salt thereof, wherein L is a bond.

Clause 42. The compound of any one of clauses 1 to 37, or a pharmaceutically acceptable salt thereof, wherein L is C₁₋₃ alkylene.

Clause 43. The compound of any one of clauses 1 to 42, or a pharmaceutically acceptable salt thereof, wherein X is CR³.

Clause 44. The compound of clause 43, or a pharmaceutically acceptable salt thereof, wherein R³ is H.

Clause 45. The compound of any one of clauses 1 to 42, or a pharmaceutically acceptable salt thereof, wherein X is N.

Clause 46. The compound of any one of clauses 1 to 45, or a pharmaceutically acceptable salt thereof, wherein R⁴ is C₁₋₆ alkyl.

Clause 47. The compound of clause 46, or a pharmaceutically acceptable salt thereof, wherein R⁴ is C₁₋₃ alkyl.

Clause 48. The compound of clause 47, or a pharmaceutically acceptable salt thereof, wherein R⁴ is methyl.

Clause 49. The compound of any one of clauses 1 to 34 having Formula (III)

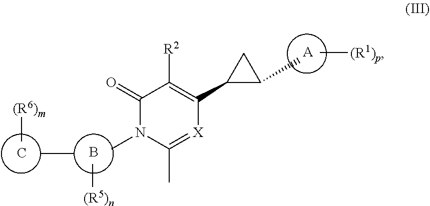

or a pharmaceutically acceptable salt thereof.

Clause 50. The compound of any one of clauses 1 to 49, or a pharmaceutically acceptable salt thereof, wherein R² is H, halogen, or C₁₋₆ alkyl.

Clause 51. The compound of clause 50, or a pharmaceutically acceptable salt thereof, wherein R² is halogen.

Clause 52. The compound of clause 51, or a pharmaceutically acceptable salt thereof, wherein R² is chloro.

Clause 53. The compound of clause 51, or a pharmaceutically acceptable salt thereof, wherein R² is bromo.

Clause 54. The compound of any one of clauses 1 to 53, or a pharmaceutically acceptable salt thereof, wherein ring \textcircled{B} is C₆₋₁₀ aryl.

Clause 55. The compound of clause 54, or a pharmaceutically acceptable salt thereof, wherein ring \textcircled{B} is phenyl.

Clause 56. The compound of any one of clauses 1 to 53, or a pharmaceutically acceptable salt thereof, wherein ring is 5- to 10-membered heteroaryl.

Clause 57. The compound of clause 56, or a pharmaceutically acceptable salt thereof, wherein ring \textcircled{B} is 5- to 6-membered heteroaryl.

Clause 58. The compound of clause 57, or a pharmaceutically acceptable salt thereof, wherein ring \textcircled{B} is 6-membered heteroaryl.

Clause 59. The compound of clause 58, or a pharmaceutically acceptable salt thereof, wherein ring \textcircled{B} is pyridinyl.

Clause 60. The compound of clause 58 having Formula (IV)

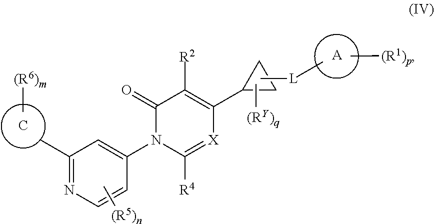

or a pharmaceutically acceptable salt thereof.

Clause 61. The compound of any one of clauses 1 to 60, or a pharmaceutically acceptable salt thereof, wherein each R⁵ is independently halogen or C₁₋₆ alkyl.

Clause 62. The compound of clause 61, or a pharmaceutically acceptable salt thereof, wherein each R⁵ is independently chloro, fluoro, or methyl.

Clause 63. The compound of any one of clauses 1 to 62, or a pharmaceutically acceptable salt thereof, wherein at least one R⁵ is C₁₋₆ alkyl.

Clause 64. The compound of any one of clauses 1 to 63, or a pharmaceutically acceptable salt thereof, wherein at least one R⁵ is methyl.

Clause 65. The compound of any one of clauses 1 to 64, or a pharmaceutically acceptable salt thereof, wherein at least one R⁵ is fluoro.

Clause 66. The compound of any one of clauses 1 to 65, or a pharmaceutically acceptable salt thereof, wherein at least one R⁵ is chloro.

Clause 67. The compound of any one of clauses 1 to 66, or a pharmaceutically acceptable salt thereof, wherein n is 1 or 2.

Clause 68. The compound of clause 67, or a pharmaceutically acceptable salt thereof, wherein n is 2.

Clause 69. The compound of any one of clauses 1 to 53, or a pharmaceutically acceptable salt thereof, wherein ring \textcircled{B} is

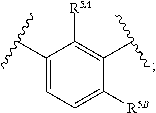

and
each R^{5A} and R^{5B} is independently H or R⁵.

Clause 70. The compound of any one of clauses 1 to 53, or a pharmaceutically acceptable salt thereof, wherein ring \textcircled{B} is

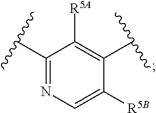

and
each R^{5A} and R^{5B} is independently H or R⁵.

Clause The compound of any one of clauses 1 to 53 having Formula (V)

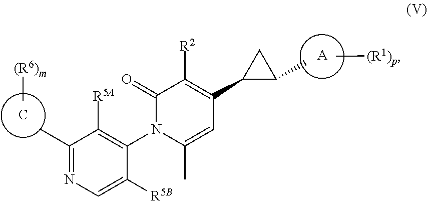

or a pharmaceutically acceptable salt thereof,
wherein each R^{5A} and R^{5B} is independently H or R⁵.

Clause 72. The compound of any one of clauses 69 to 71, or a pharmaceutically acceptable salt thereof, wherein R^{5B} is halogen, C₁₋₆ alkyl, or C₂₋₆ alkynyl, wherein each alkyl and alkynyl is optionally substituted with one to three halogen.

Clause 73. The compound of any one of clauses 69 to 72, or a pharmaceutically acceptable salt thereof, wherein R^{5B} is methyl, —CF₃, CHF₂, or —C≡CCH₃.

Clause 74. The compound of any one of clauses 69 to 72, or a pharmaceutically acceptable salt thereof, wherein R^{5B} is chloro.

Clause 75. The compound of any one of clauses 69 to 72, or a pharmaceutically acceptable salt thereof, wherein R^{5B} is C₁₋₃ alkyl.

Clause 76. The compound of any one of clauses 69 to 73, or a pharmaceutically acceptable salt thereof, wherein R^{5B} is methyl.

Clause 77. The compound of any one of clauses 69 to 71, or a pharmaceutically acceptable salt thereof, wherein R^{5B} is H.

Clause 78. The compound of any one of clauses 1 to 53, or a pharmaceutically acceptable salt thereof, wherein ring \textcircled{B} is

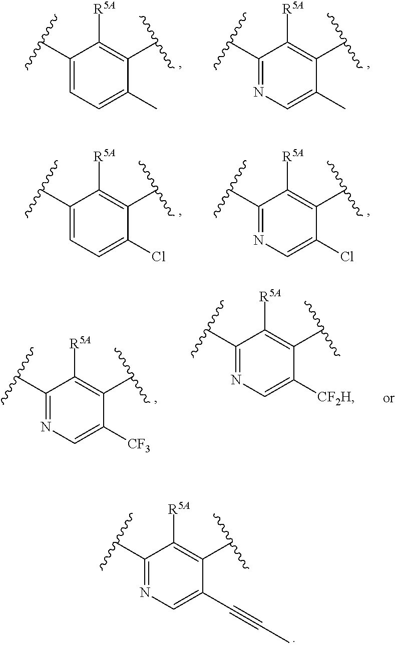

Clause 79. The compound of any one of clauses 1 to 78, or a pharmaceutically acceptable salt thereof, wherein R^{5A} is halogen.

Clause 80. The compound of any one of clauses 1 to 79, or a pharmaceutically acceptable salt thereof, wherein R^{1A} is fluoro.

Clause 81. The compound of any one of clauses 1 to 78, or a pharmaceutically acceptable salt thereof, wherein R^{1A} is H.

Clause 82. The compound of any one of clauses 1 to 53, or a pharmaceutically acceptable salt thereof, wherein ring \textcircled{B} is

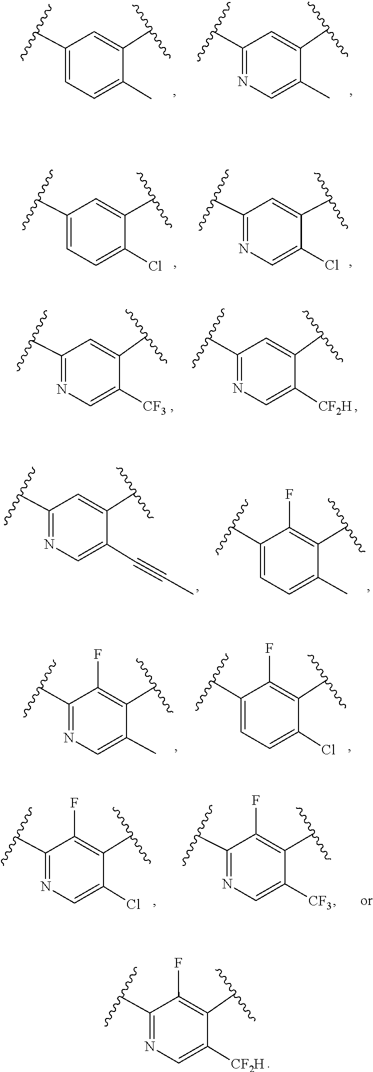

Clause 83. The compound of any one of clauses to 53, or a pharmaceutically acceptable salt thereof, wherein ring \textcircled{B} is

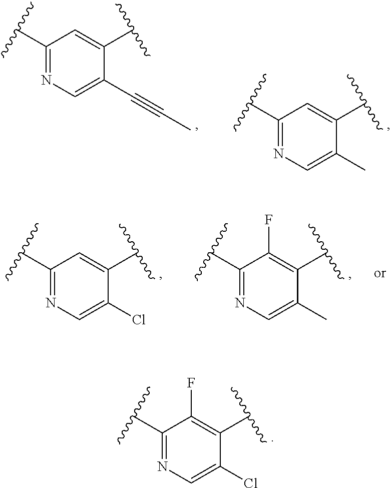

Clause 84. The compound of any one of clauses 1 to 53, or a pharmaceutically acceptable salt thereof, wherein ring \textcircled{B} is

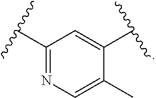 [SMILES: *c1cc(*)c(C)cn1]

Clause 85. The compound of any one of clauses 1 to 53, or a pharmaceutically acceptable salt thereof, wherein ring \textcircled{B} is

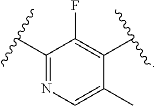 [SMILES: *c1ncc(C)c(*)c1F]

Clause 86. The compound of any one of clauses 1 to 85, or a pharmaceutically acceptable salt thereof, wherein ring \textcircled{C} is C₆₋₁₀ aryl.

Clause 87. The compound of any one of clauses 1 to 85, or a pharmaceutically acceptable salt thereof, wherein \textcircled{C} ring is phenyl.

Clause 88. The compound of any one of clauses 1 to 85, or a pharmaceutically acceptable salt thereof, wherein ring \textcircled{C} is 5- to 10-membered heteroaryl.

Clause 89. The compound of any one of clauses 1 to 85, or a pharmaceutically acceptable salt thereof, wherein ring \textcircled{C} is 5- to 6-membered heteroaryl.

Clause 90. The compound of any one of clauses 1 to 85, or a pharmaceutically acceptable salt thereof, wherein ring \textcircled{C} is

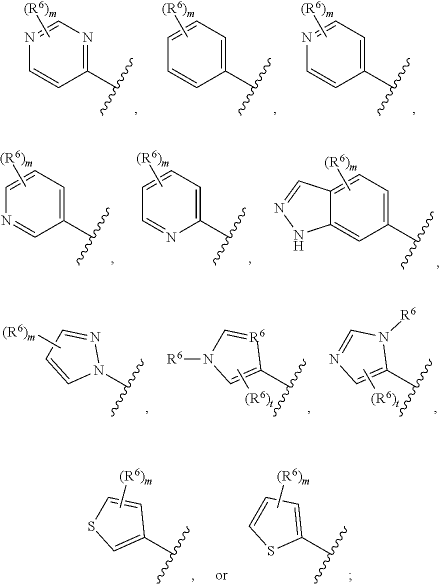

wherein
t is 0, 1, or 2.

Clause 91. The compound of any one of clauses 1 to 85, or a pharmaceutically acceptable salt thereof, wherein ring \textcircled{C} is

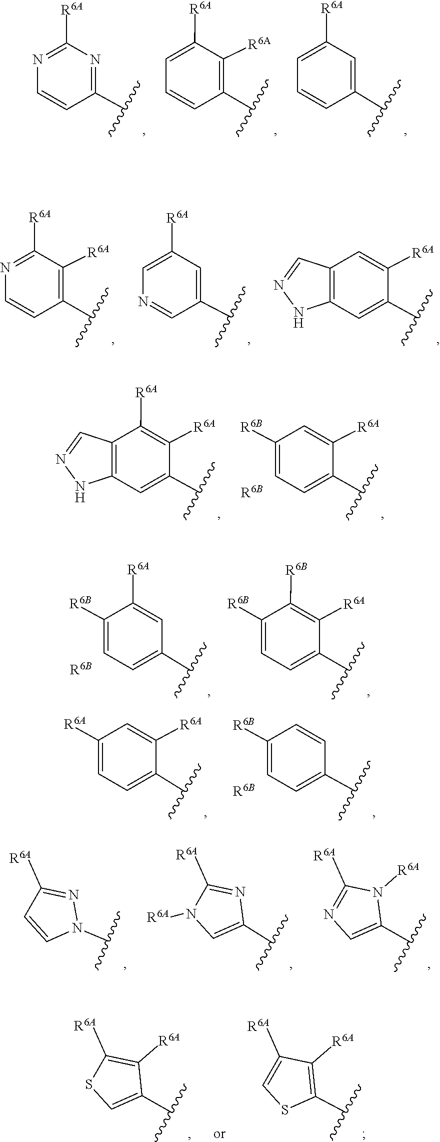

wherein
each R^{6A} is independently oxo, halogen, —NO₂, —N₃, —CN, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, 5- to 10-membered heteroaryl, —OR^{6a1}N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, —N(R^{6a1})C(O)N(R^{6a1})₂—N═S(O)(R^{6a1})z, —N(R^{6a1})S(O)₂ (R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂—Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, wherein each alkyl, alkenyl, alkynyl, cycloalkyl, heterocyclyl, aryl, and heteroaryl is optionally substituted with one to three R^{6b1}; and
two R^{6B} groups taken together with two adjacent atoms of ring \textcircled{C} to which they are attached forma 4- to 10-membered heterocyclyl, wherein the heterocyclyl is optionally substituted with one to three R^{6b1}.

Clause 92. The compound of any one of clauses 1 to 85, or a pharmaceutically acceptable salt thereof, wherein ring \textcircled{C} is

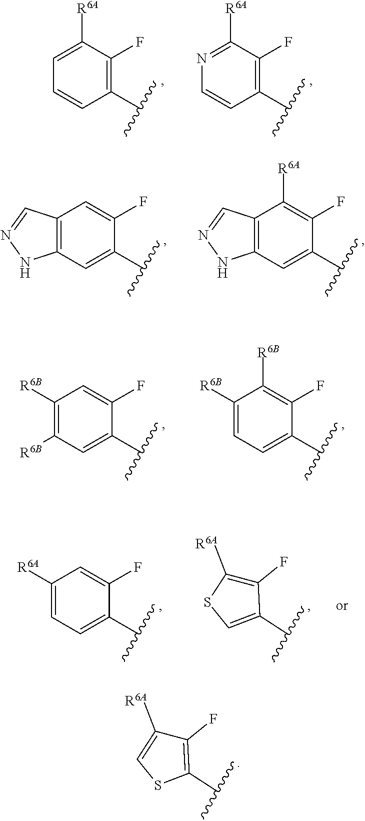

Clause 93. The compound of any one of clauses 1 to 85, or a pharmaceutically acceptable salt thereof, wherein ring \textcircled{C} is

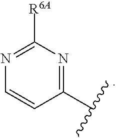

Clause 94. The compound of any one of clauses 1 to 85, or a pharmaceutically acceptable salt thereof, wherein ring \textcircled{C} is

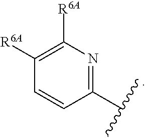

Clause 95. The compound of any one of clauses 1 to 85, or a pharmaceutically acceptable salt thereof, wherein ring \textcircled{C} is

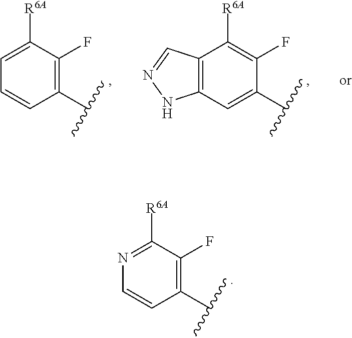

Clause 96. The compound of any one of clauses 1 to 85, or a pharmaceutically acceptable salt thereof, wherein ring \textcircled{C} is

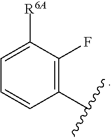

Clause 97. The compound of any one of clauses 1 to 96, or a pharmaceutically acceptable salt thereof, wherein each R⁶ is independently halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to three R^{6b1}.

Clause 98. The compound of any one of clauses 1 to 97, or a pharmaceutically acceptable salt thereof, wherein each R⁶ is independently halogen, —CN, —C₁₋₆ alkyl, 4- to 10-membered heterocyclyl, 5- to 10-membered heteroaryl, —C(O)N(R^{6a1})(R^{6a1}), —N(R^{6a1})C(O)—R^{6a1}, —N(R^{6a1})C(O)O—R^{6a1}, N═S(O)(R^{6a1})₂, —S(NR^{6a1})(O)(R^{6a1}), or —S(O)₂R^{6a1}.

Clause 99. The compound of any one of clauses 1 to 98, or a pharmaceutically acceptable salt thereof, wherein at least one R⁶ is halogen.

Clause 100. The compound of any one of clauses 1 to 85 having Formula (VI)

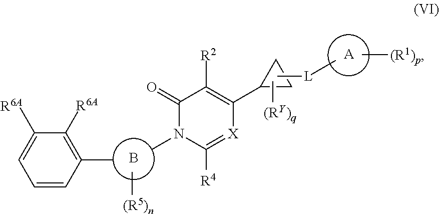

or a pharmaceutically acceptable salt thereof,
wherein each R^{6A} is independently H or R⁶.

Clause 101. The compound of clause 100 having Formula (VII)

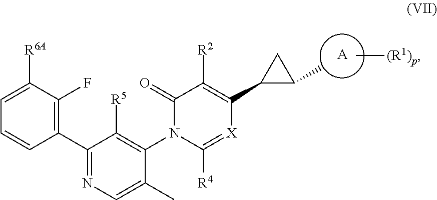

or a pharmaceutically acceptable salt thereof.

Clause 102. The compound of clause 100 or 101, or a pharmaceutically acceptable salt thereof, wherein each R^{6A} is halogen, —CN, —OH, —C₁₋₆ alkyl, C₃₋₁₀ cycloalkyl, 4- to 10-membered heterocyclyl, 5- to 10-membered heteroaryl, —O^{R6a1}, —C(O)N(R^{6a1})(R^{6a1}), —N(R^{6a1})C(O)—R^{6a1}, —N(R^{6a1})C(O)O—R^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to three R^{6b1}.

Clause 103. The compound of any one of clauses 100 to 102, or a pharmaceutically acceptable salt thereof, wherein R^{6A} is C₁₋₆ alkyl, C₃₋₁₀ cycloalkyl, 4- to 10-membered heterocyclyl, 5- to 10-membered heteroaryl, —C(O)N(R^{6a1})₂, —N(R^{6a1})C(O)R^{6a1}, —N(R^{6a1})C(O)OR^{6a1}, —OC(O)N(R^{6a1})(R^{6a1}), —N═S(O)(R^{6a1})₂, —S(O)R^{6a1}, —S(NR^{6a1})(O)(R^{6a1}), —S(O)₂R^{6a1}, or —S(O)₂N(R^{6a1})₂, wherein each alkyl, cycloalkyl, heterocyclyl, and heteroaryl is optionally substituted with one to three halogen and is further optionally substituted with one to three R^{6b1}.

Clause 104. The compound of any one of clauses 100 to 102, or a pharmaceutically acceptable salt thereof, wherein R^{6A} is 5- to 6-membered heterocyclyl or 5- to 6-membered heteroaryl.

Clause 105. The compound of any one of clauses 100 to 102, or a pharmaceutically acceptable salt thereof, wherein R^{6A} is —C(O)N(R^{6a1})₂.

Clause 106. The compound of any one of clauses 100 to 102, or a pharmaceutically acceptable salt thereof, wherein R^{6A} is —N(R^{6a1})C(O)R^{6a1}.

Clause 107. The compound of any one of clauses 100 to 102, or a pharmaceutically acceptable salt thereof, wherein R^{6A} is —N(R^{6a1})C(O)OR^{6a1}.

Clause 108. The compound of any one of clauses 100 to 102, or a pharmaceutically acceptable salt thereof, wherein R^{6A} is —NHC(O)OR^{6a1}.

Clause 109. The compound of any one of clauses 100 to 102, or a pharmaceutically acceptable salt thereof, wherein R^{6A} is —S(O)₂R^{6a1}.

Clause 110. The compound of any one of clauses 100 to 102, or a pharmaceutically acceptable salt thereof, wherein R^{6A} is —N═S(O)(R^{6a1})₂.

Clause 111. The compound of any one of clauses 100 to 102, or a pharmaceutically acceptable salt thereof, wherein R^{6A} is —S(NR^{6a1})(O)(R^{6a1}).

Clause 112. The compound of any one of clauses 100 to 102, or a pharmaceutically acceptable salt thereof, wherein R^{6A} is C₁₋₆ alkyl, wherein the alkyl is substituted with one to three R^{6b1}.

Clause 113. The compound of any one of clauses 100 to 102, or a pharmaceutically acceptable salt thereof, wherein R^{6A} is C₃₋₆ cycloalkyl, wherein the cycloalkyl is substituted with one to three R^{6b1}.

Clause 114. The compound of any one of clauses 1 to 101, or a pharmaceutically acceptable salt thereof, wherein ring \textcircled{C} is

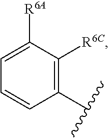

wherein R^{6c} is halogen.

Clause 115. The compound of clause 114, or a pharmaceutically acceptable salt thereof, wherein R^{6C} is fluoro.

Clause 116. The compound of any one of clauses 1 to 115, or a pharmaceutically acceptable salt thereof, wherein each R^{6a1} is independently H, C₁₋₆ alkyl, C₃₋₁₀ cycloalkyl, or 4- to 10-membered heterocyclyl, or two R^{6a1} groups taken together with the atom or atoms to which they are attached form a 4- to 10-membered heterocyclyl, wherein each alkyl, cycloalkyl, and heterocyclyl is optionally substituted with one to three R^{6b1}.

Clause 117. The compound of any one of clauses 1 to 116, or a pharmaceutically acceptable salt thereof, wherein each R^{6a1} is independently H, C₁₋₆ alkyl, or C₃₋₆ cycloalkyl, wherein each alkyl and cycloalkyl is optionally substituted with one to three R^{6b1}.

Clause 118. The compound of any one of clauses 1 to 117, or a pharmaceutically acceptable salt thereof, wherein each R^{6a1} is independently H or C₁₋₃ alkyl, wherein each alkyl is optionally substituted with one to three R^{6b1}.

Clause 119. The compound of any one of clauses 1 to 118, or a pharmaceutically acceptable salt thereof, wherein each R^{6a1} is independently H or C₃ cycloalkyl, wherein the cycloalkyl is optionally substituted with one to three R^{6b1}.

Clause 120. The compound of any one of clauses 1 to 116, or a pharmaceutically acceptable salt thereof, wherein each R^{6a1} is independently H, C₁₋₆ alkyl, C₃₋₁₀ cycloalkyl, or 4- to 10-membered heterocyclyl.

Clause 121. The compound of any one of clauses 1 to 116 and 120, or a pharmaceutically acceptable salt thereof, wherein each R^{6a1} is independently H, C₁₋₃ alkyl, or C₃₋₆ cycloalkyl.

Clause 122. The compound of any one of clauses 1 to 116 and 120 to 121, or a pharmaceutically acceptable salt thereof, wherein each R^{6a1} is independently H or methyl.

Clause 123. The compound of any one of clauses 1 to 116 and 120 to 122, or a pharmaceutically acceptable salt thereof, wherein each R^{6a1} is methyl.

Clause 124. The compound of any one of clauses 1 to 116, or a pharmaceutically acceptable salt thereof, wherein two R^{6a1} groups taken together with the atom or atoms to which they are attached form a 4- to 6-membered heterocyclyl.

Clause 125. The compound of any one of clauses 1 to 116 and 124, or a pharmaceutically acceptable salt thereof, wherein two R^{6a1} groups taken together with the nitrogen atom to which they are attached form a 4- to 6-membered heterocyclyl.

Clause 126. The compound of any one of clauses 1 to 123, or a pharmaceutically acceptable salt thereof, wherein each R^{6b1} is independently halogen, —CN, C₁₋₆ alkyl, C₃₋₁₀ cycloalkyl, 4- to 10-membered heterocyclyl, —OR^{6b2}, —N(R^{6b2})₂, N(R¹²)C(O)N(R^{6b2})₂, —N(R^{6b2})S(O)₂(R^{6b2}), —S(O)₂R^{6b2}, or —S(O)₂N(R^{6b2})₂ wherein each alkyl and cycloalkyl is optionally substituted with one to three R^{6c1}.

Clause 127. The compound of any one of clauses 1 to 123, or a pharmaceutically acceptable salt thereof, wherein each R^{6b1} is independently halogen, —CN, C₁₋₆ alkyl, 4- to 10-membered heterocyclyl, —OR^{6b2}, —N(R^{6b2})₂, N(R^{6b2})C(O)N(R^{6b2})₂, —N(R^{6b2})S(O)₂(R^{6b2}), or —S(O)₂R^{6b2}, wherein each alkyl and cycloalkyl is optionally substituted with one to three R^{6c1}.

Clause 128. The compound of any one of clauses 1 to 123, or a pharmaceutically acceptable salt thereof, wherein each R^{6b1} is independently halogen, C₁₋₆ alkyl, —OR^{6b2}, or —S(O)₂R^{6b2}.

Clause 129. The compound of any one of clauses 1 to 123, or a pharmaceutically acceptable salt thereof, wherein each R^{6b1} is independently fluoro, —OH, or methyl.

Clause 130. The compound of any one of clauses 1 to 123, or a pharmaceutically acceptable salt thereof, wherein each R^{6b1} is fluoro.

Clause 131. The compound of any one of clauses 1 to 123, or a pharmaceutically acceptable salt thereof, wherein each R^{6b1} is —OH.

Clause 132. The compound of any one of clauses 1 to 123, or a pharmaceutically acceptable salt thereof, wherein each R^{6b1} is methyl.

Clause 133. The compound of any one of clauses 1 to 132, or a pharmaceutically acceptable salt thereof, wherein each R^{6b2} is independently H or C₁₋₃ alkyl.

Clause 134. The compound ofany one of clauses to 133, or a pharmaceutically acceptable salt thereof, wherein each R^{6c1} and R^{60Z} is independently halogen, —OH, or —NH₂.

Clause 135. The compound of any one of clauses 1 to 134, or a pharmaceutically acceptable salt thereof, wherein each R^{6d1} is independently C₆ alkyl, C₃′ cycloalkyl, 4- to 10-membered heterocyclyl, C₆₋₁₀ aryl, or 5- to 10-membered heteroaryl.

Clause 136. The compound of any one of clauses 1 to 135, or a pharmaceutically acceptable salt thereof, wherein each R^{6d1} is independently C₆ alkyl.

Clause 137. The compound ofany one of clauses 1 to 136, or a pharmaceutically acceptable salt thereof, wherein m is 1, 2, or 3.

Clause 138. The compound of any one of clauses 1 to 137, or a pharmaceutically acceptable salt thereof, wherein m is 1 or 2.

Clause 139. The compound of any one of clauses 1 to 138, or a pharmaceutically acceptable salt thereof, wherein the compound is selected from:

4-((1S,2R)-2-benzylcyclopropyl)-3-chloro-2′-(2-(2-hydroxypropan-2-yl)pyrimidin-4-yl)-5′,6-
dimethyl-2H-[1,4′-bipyridin]-2-one
3-chloro-2′-(2-(2-hydroxypropan-2-yl)pyrimidin-4-yl)-4-((1S,2S)-2-(3-
methoxyphenyl)cyclopropyl)-5′,6-dimethyl-2H-[1,4′-bipyridin]-2-one
3-chloro-4-((1S,2S)-2-(4-fluorophenyl)cyclopropyl)-2′-(2-(2-hydroxypropan-2-yl)pyrimidin-
4-yl)-5′,6-dimethyl-2H-[1,4′-bipyridin]-2-one
3-chloro-4-((1S,2S)-2-(4-fluorophenyl)cyclopropyl)-2′-(2-(2-hydroxypropan-2-yl)pyrimidin-
4-yl)-5′,6-dimethyl-2H-[1,4′-bipyridin]-2-one
3-chloro-4-((1R,2R)-2-(4-fluorophenyl)cyclopropyl)-2′-(2-(2-hydroxypropan-2-yl)pyrimidin-
4-yl)-5′,6-dimethyl-2H-[1,4′-bipyridin]-2-one
3-chloro-4-((1S,2S)-2-(2,4-difluorophenyl)cyclopropyl)-2′-(2-(2-hydroxypropan-2-
yl)pyrimidin-4-yl)-5′,6-dimethyl-2H-[1,4′-bipyridin]-2-one
3-chloro-4-((1S,2S)-2-(3,4-difluorophenyl)cyclopropyl)-2′-(2-(2-hydroxypropan-2-
yl)pyrimidin-4-yl)-5′,6-dimethyl-2H-[1,4′-bipyridin]-2-one
3-chloro-4-((1S,2S)-2-(2,2-difluorobenzo[d][1,3]dioxol-5-yl)cyclopropyl)-2′-(2-(2-
hydroxypropan-2-yl)pyrimidin-4-yl)-5′,6-dimethyl-2H-[1,4′-bipyridin]-2-one
3-chloro-4-((1S,2S)-2-(4-chloro-3-fluorophenyl)cyclopropyl)-2′-(2-(2-hydroxypropan-2-
yl)pyrimidin-4-yl)-5′,6-dimethyl-2H-[1,4′-bipyridin]-2-one
3-chloro-4-((1S,2S)-2-(2,6-difluorophenyl)cyclopropyl)-2′-(2-(2-hydroxypropan-2-
yl)pyrimidin-4-yl)-5′,6-dimethyl-2H-[1,4′-bipyridin]-2-one
3-chloro-4-((1S,2S)-2-(3-chloro-4-fluorophenyl)cyclopropyl)-2′-(2-(2-hydroxypropan-2-
yl)pyrimidin-4-yl)-5′,6-dimethyl-2H-[1,4′-bipyridin]-2-one
3-chloro-4-((1S,2S)-2-(2-chlorophenyl)cyclopropyl)-2′-(2-(2-hydroxypropan-2-yl)pyrimidin-
4-yl)-5′,6-dimethyl-2H-[1,4′-bipyridin]-2-one
3-chloro-4-((1S,2S)-2-(4-cyclopropylphenyl)cyclopropyl)-2′-(2-(2-hydroxypropan-2-
yl)pyrimidin-4-yl)-5′,6-dimethyl-2H-[1,4′-bipyridin]-2-one
3-chloro-4-((1S,2S)-2-(5-(difluoromethoxy)pyridin-2-yl)cyclopropyl)-2′-(2-(2-
hydroxypropan-2-yl)pyrimidin-4-yl)-5′,6-dimethyl-2H-[1,4′-bipyridin]-2-one
3-chloro-2′-(2-(2-hydroxypropan-2-yl)pyrimidin-4-yl)-5′,6-dimethyl-4-((1S,2S)-2-(2,3,4-
trifluorophenyl)cyclopropyl)-2H-[1,4′-bipyridin]-2-one
3-chloro-4-((1R,2R)-2-(1-(difluoromethyl)-1H-pyrazol-4-yl)cyclopropyl)-2′-(2-(2-
hydroxypropan-2-yl)pyrimidin-4-yl)-5′,6-dimethyl-2H-[1,4′-bipyridin]-2-one
3-chloro-4-((1S,2S)-2-(1-(difluoromethyl)-1H-pyrazol-4-yl)cyclopropyl)-2′-(2-(2-
hydroxypropan-2-yl)pyrimidin-4-yl)-5′,6-dimethyl-2H-[1,4′-bipyridin]-2-one
3-chloro-4-((1S,2S)-2-(6-cyclopropylpyridin-2-yl)cyclopropyl)-2′-(2-(2-hydroxypropan-2-
yl)pyrimidin-4-yl)-5′,6-dimethyl-2H-[1,4′-bipyridin]-2-one
3-chloro-2′-(2-(2-hydroxypropan-2-yl)pyrimidin-4-yl)-5′,6-dimethyl-4-((1S,2S)-2-(2-
methylthiazol-4-yl)cyclopropyl)-2H-[1,4′-bipyridin]-2-one
4-((1S,2S)-2-(3-chloro-2′-(2-(2-hydroxypropan-2-yl)pyrimidin-4-yl)-5′,6-dimethyl-2-oxo-2H-
[1,4′-bipyridin]-4-yl)cyclopropyl)-2-fluorobenzonitrile
4-((1S,2S)-2-(3-chloro-2′-(2-(2-hydroxypropan-2-yl)pyrimidin-4-yl)-5′,6-dimethyl-2-oxo-2H-
[1,4′-bipyridin]-4-yl)cyclopropyl)benzonitrile
3-chloro-2′-(2-(2-hydroxypropan-2-yl)pyrimidin-4-yl)-5′,6-dimethyl-4-((1S,2S)-2-(5-
(trifluoromethyl)pyridin-2-yl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(6-
(trifluoromethyl)pyridin-3-yl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(5-
(trifluoromethoxy)pyridin-2-yl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(6-
(trifluoromethoxy)pyridin-3-yl)cyclopropyl)-2H-[1,4′-bipyridin]-2-one
3-chloro-2′-(2-(2-hydroxypropan-2-yl)pyrimidin-4-yl)-5′,6-dimethyl-4-((1R,2R)-2-(2-
(trifluoromethyl)thiazol-5-yl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(2-
(trifluoromethyl)thiazol-5-yl)cyclopropyl)-2H-[1,4′-bipyridin]-2-one
3-chloro-4-((1S,2S)-2-(4-chloro-2-fluorophenyl)cyclopropyl)-2′-(2-(2-hydroxypropan-2-
yl)pyrimidin-4-yl)-5′,6-dimethyl-2H-[1,4′-bipyridin]-2-one
3-chloro-2′-(2-(2-hydroxypropan-2-yl)pyrimidin-4-yl)-5′,6-dimethyl-4-((1S,2S)-2-(3-
(trifluoromethoxy)phenyl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(4-
(trifluoromethyl)phenyl)cyclopropyl)-2H-[1,4′-bipyridin]-2-one
4-((1S,2S)-2-(3-chloro-2′-(2-(2-hydroxypropan-2-yl)pyrimidin-4-yl)-5′,6-dimethyl-2-oxo-2H-
[1,4′-bipyridin]-4-yl)cyclopropyl)picolinonitrile
5-((1S,2S)-2-(3-chloro-2′-(2-(2-hydroxypropan-2-yl)pyrimidin-4-yl)-5′,6-dimethyl-2-oxo-2H-
[1,4′-bipyridin]-4-yl)cyclopropyl)nicotinonitrile
3-chloro-2′-(2-(2-hydroxypropan-2-yl)pyrimidin-4-yl)-5′,6-dimethyl-4-((1R,2R)-2-(5-
(trifluoromethyl)pyridin-3-yl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(5-
(trifluoromethyl)pyridin-3-yl)cyclopropyl)-2H-[1,4′-bipyridin]-2-one
methyl 3-((1S,2S)-2-(3-chloro-2′-(2-(2-hydroxypropan-2-yl)pyrimidin-4-yl)-5′,6-dimethyl-2-
oxo-2H-[1,4′-bipyridin]-4-yl)cyclopropyl)benzoate
3-chloro-4-((1S,3S)-2,2-difluoro-3-phenylcyclopropyl)-2′-(2-(2-hydroxypropan-2-
yl)pyrimidin-4-yl)-5′,6-dimethyl-2H-[1,4′-bipyridin]-2-one
3-chloro-2′-(2-(2-hydroxypropan-2-yl)pyrimidin-4-yl)-5′,6-dimethyl-4-((1S,2S)-2-(2,3,6-
trifluorophenyl)cyclopropyl)-2H-[1,4′-bipyridin]-2-one
3-chloro-4-((1S,2S)-2-(4-fluoro-3-(trifluoromethyl)phenyl)cyclopropyl)-2′-(2-(2-
hydroxypropan-2-yl)pyrimidin-4-yl)-5′,6-dimethyl-2H-[1,4′-bipyridin]-2-one
3-chloro-2′-(2-(2-hydroxypropan-2-yl)pyrimidin-4-yl)-5′,6-dimethyl-4-((1S,2S)-2-
phenylcyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(2-
(trifluoromethyl)pyrimidin-4-yl)cyclopropyl)-2H-[1,4′-bipyridin]-2-one
3-chloro-2′-(2-(2-hydroxypropan-2-yl)pyrimidin-4-yl)-5′,6-dimethyl-4-((1R,2R)-2-(2-
(trifluoromethyl)pyrimidin-4-yl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(pyridin-2-
yl)cyclopropyl)-2H-[1,4′-bipyridin]-2-one
3-chloro-4-((1S,2S)-2-(1-(difluoromethyl)-1H-pyrazol-3-yl)cyclopropyl)-2′-(2-(2-
hydroxypropan-2-yl)pyrimidin-4-yl)-5′,6-dimethyl-2H-[1,4′-bipyridin]-2-one
3-chloro-4-((1S,2R)-2-(4,4-difluorocyclohexyl)cyclopropyl)-2′-(2-(2-hydroxypropan-2-
yl)pyrimidin-4-yl)-5′,6-dimethyl-2H-[1,4′-bipyridin]-2-one
3-chloro-4-((1S,2S)-2-(1-(difluoromethyl)-1H-pyrazol-4-yl)cyclopropyl)-2′-(2-(2-
hydroxypropan-2-yl)pyrimidin-4-yl)-5′,6-dimethyl-2H-[1,4′-bipyridin]-2-one
3-chloro-2′-(2-(2-hydroxypropan-2-yl)pyrimidin-4-yl)-4-((1S,2S)-2-(6-methoxypyridin-2-
yl)cyclopropyl)-5′,6-dimethyl-2H-[1,4′-bipyridin]-2-one
3-((1S,2S)-2-(3-chloro-2′-(2-(2-hydroxypropan-2-yl)pyrimidin-4-yl)-5′,6-dimethyl-2-oxo-2H-
[1,4′-bipyridin]-4-yl)cyclopropyl)-4-fluorobenzonitrile
3-chloro-4-(2-(2-fluorophenyl)cyclopropyl)-2′-(2-(2-hydroxypropan-2-yl)pyrimidin-4-yl)-
5′,6-dimethyl-2H-[1,4′-bipyridin]-2-one
3-chloro-4-(2-(3-fluorophenyl)cyclopropyl)-2′-(2-(2-hydroxypropan-2-yl)pyrimidin-4-yl)-
5′,6-dimethyl-2H-[1,4′-bipyridin]-2-one
3-(2-(3-chloro-2′-(2-(2-hydroxypropan-2-yl)pyrimidin-4-yl)-5′,6-dimethyl-2-oxo-2H-[1,4′-
bipyridin]-4-yl)cyclopropyl)benzonitrile
3-(2-(3-chloro-2′-(2-(2-hydroxypropan-2-yl)pyrimidin-4-yl)-5′,6-dimethyl-2-oxo-2H-[1,4′-
bipyridin]-4-yl)cyclopropyl)-5-fluorobenzonitrile
3-chloro-4-((1S,2S)-2-(3-fluoropyridin-4-yl)cyclopropyl)-2′-(2-(2-hydroxypropan-2-
yl)pyrimidin-4-yl)-5′,6-dimethyl-2H-[1,4′-bipyridin]-2-one
3-chloro-4-((1S,2S)-2-(5-fluoropyridin-3-yl)cyclopropyl)-2′-(2-(2-hydroxypropan-2-
yl)pyrimidin-4-yl)-5′,6-dimethyl-2H-[1,4′-bipyridin]-2-one
3-chloro-4-(2′,3′-dihydrospiro[cyclopropane-1,1′-inden]-2-yl)-2′-(2-(2-hydroxypropan-2-
yl)pyrimidin-4-yl)-5′,6-dimethyl-2H-[1,4′-bipyridin]-2-one
3-chloro-4-((1S,2S)-2-(3,4-difluorophenyl)cyclopropyl)-2′-(2-(2-hydroxypropan-2-
yl)pyrimidin-4-yl)-5′,6-dimethyl-2H-[1,4′-bipyridin]-2-one
3-chloro-2′-(2-(2-hydroxypropan-2-yl)pyrimidin-4-yl)-5′,6-dimethyl-4-((1S,2S)-2-(3,4,5-
trifluorophenyl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(2,4,5-
trifluorophenyl)cyclopropyl)-2H-[1,4′-bipyridin]-2-one
3-chloro-4-((1S,2S)-2-(3,5-difluorophenyl)cyclopropyl)-2′-(2-(2-hydroxypropan-2-
yl)pyrimidin-4-yl)-5′,6-dimethyl-2H-[1,4′-bipyridin]-2-one
3-chloro-2′-(2-(2-hydroxypropan-2-yl)pyrimidin-4-yl)-5′,6-dimethyl-4-((1S,2S)-2-(3-
(trifluoromethyl)phenyl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(1-
(trifluoromethyl)-1H-pyrazol-4-yl)cyclopropyl)-2H-[1,4′-bipyridin]-2-one
3-chloro-2′-(2-(2-hydroxypropan-2-yl)pyrimidin-4-yl)-5′,6-dimethyl-4-((1R,2R)-2-(1-
(trifluoromethyl)-1H-pyrazol-4-yl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(2,4,6-
trifluorophenyl)cyclopropyl)-2H-[1,4′-bipyridin]-2-one
3-chloro-2′-(2-(2-hydroxypropan-2-yl)pyrimidin-4-yl)-5′,6-dimethyl-4-((1S,2S)-2-(5-
(trifluoromethyl)pyrazin-2-yl)cyclopropyl)-2H-[1,4′-bipyridin]-2-one
3-chloro-3′-fluoro-4-((1S,2S)-2-(4-fluorophenyl)cyclopropyl)-2′-(2-(2-hydroxypropan-2-
yl)pyrimidin-4-yl)-5′,6-dimethyl-2H-[1,4′-bipyridin]-2-one
4-((1S,2S)-2-(3-amino-4-fluorophenyl)cyclopropyl)-3-chloro-2′-(2-(2-hydroxypropan-2-
yl)pyrimidin-4-yl)-5′,6-dimethyl-2H-[1,4′-bipyridin]-2-one
N-(5-((1S,2S)-2-(3-chloro-2′-(2-(2-hydroxypropan-2-yl)pyrimidin-4-yl)-5′,6-dimethyl-2-oxo-
2H-[1,4′-bipyridin]-4-yl)cyclopropyl)-2-fluorophenyl)acetamide
N-(5-((1S,2S)-2-(3-chloro-2′-(2-(2-hydroxypropan-2-yl)pyrimidin-4-yl)-5′,6-dimethyl-2-oxo-
2H-[1,4′-bipyridin]-4-yl)cyclopropyl)-2-fluorophenyl)cyclopropanecarboxamide
3-(3-chloro-4-((1S,2S)-2-(4-fluorophenyl)cyclopropyl)-5′,6-dimethyl-2-oxo-2H-[1,4′-
bipyridin]-2′-yl)-2-fluoro-N,N-dimethylbenzamide
3-(3-chloro-4-(2-(3,4-difluorophenyl)-2-methylcyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(5-chloropyridin-3-yl)cyclopropyl)-5′,6-dimethyl-2-oxo-2H-[1,4′-
bipyridin]-2′-yl)-2-fluoro-N,N-dimethylbenzamide
3-(3-chloro-5′,6-dimethyl-2-oxo-4-((1S,2S)-2-(o-tolyl)cyclopropyl)-2H-[1,4′-bipyridin]-2′-
yl)-2-fluoro-N,N-dimethylbenzamide
3-(3-chloro-4-((1S,2S)-2-(2,6-difluorophenyl)cyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(4-chlorophenyl)cyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(3-chlorophenyl)cyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(2,5-difluorophenyl)cyclopropyl)-5′,6-dimethyl-2-oxo-2H-[1,4′-
bipyridin]-2′-yl)-2-fluoro-N,N-dimethylbenzamide
3-(3-chloro-5′,6-dimethyl-4-((1S,2S)-2-(1-methyl-1H-pyrazol-3-yl)cyclopropyl)-2-oxo-2H-
[1,4′-bipyridin]-2′-yl)-2-fluoro-N,N-dimethylbenzamide
3-(4-((1S,2S)-2-(1-(tert-butyl)-1H-pyrazol-4-yl)cyclopropyl)-3-chloro-5′,6-dimethyl-2-oxo-
2H-[1,4′-bipyridin]-2′-yl)-2-fluoro-N,N-dimethylbenzamide
3-(3-chloro-4-((1S,2S)-2-(5-chloro-3-fluoropyridin-2-yl)cyclopropyl)-5′,6-dimethyl-2-oxo-
2H-[1,4′-bipyridin]-2′-yl)-2-fluoro-N,N-dimethylbenzamide
3-(3-chloro-4-((1S,2S)-2-(5-(difluoromethyl)pyridin-3-yl)cyclopropyl)-5′,6-dimethyl-2-oxo-
2H-[1,4′-bipyridin]-2′-yl)-2-fluoro-N,N-dimethylbenzamide
3-(3,5′-dichloro-4-((1S,2S)-2-(4-fluorophenyl)cyclopropyl)-6-methyl-2-oxo-2H-[1,4′-
bipyridin]-2′-yl)-2-fluoro-N,N-dimethylbenzamide
3-(3,5′-dichloro-4-((1S,2S)-2-(5-fluoropyridin-3-yl)cyclopropyl)-6-methyl-2-oxo-2H-[1,4′-
bipyridin]-2′-yl)-2-fluoro-N,N-dimethylbenzamide
3-(3,3′-dichloro-4-((1S,2S)-2-(4-fluorophenyl)cyclopropyl)-6-methyl-2-oxo-2H-[1,4′-
bipyridin]-2′-yl)-2-fluoro-N,N-dimethylbenzamide
3-(3,5′-dichloro-4-((1S,2S)-2-(5-chloropyridin-3-yl)cyclopropyl)-6-methyl-2-oxo-2H-[1,4′-
bipyridin]-2′-yl)-2-fluoro-N,N-dimethylbenzamide
3-(3-chloro-5′,6-dimethyl-2-oxo-4-((1S,2S)-2-(5-(trifluoromethyl)pyridin-3-yl)cyclopropyl)-
2H-[1,4′-bipyridin]-2′-yl)-2-fluoro-N,N-dimethylbenzamide
3-(3-chloro-4-((1S,2S)-2-(5-cyanopyridin-3-yl)cyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(4-fluoro-3-(2-hydroxypropan-2-yl)phenyl)cyclopropyl)-5′,6-
dimethyl-2-oxo-2H-[1,4′-bipyridin]-2′-yl)-2-fluoro-N,N-dimethylbenzamide
3-(3-chloro-4-((1S,2S)-2-(6-chloropyridin-2-yl)cyclopropyl)-5′,6-dimethyl-2-oxo-2H-[1,4′-
bipyridin]-2′-yl)-2-fluoro-N,N-dimethylbenzamide
N-(3-(3-chloro-4-((1S,2S)-2-(1-(difluoromethyl)-1H-pyrazol-4-yl)cyclopropyl)-5′,6-
dimethyl-2-oxo-2H-[1,4′-bipyridin]-2′-yl)-2-fluorophenyl)-1-methylcyclopropane-1-carboxamide
N-(3-(3-chloro-5′,6-dimethyl-2-oxo-4-((1S,2S)-2-(5-(trifluoromethyl)pyridin-3-
yl)cyclopropyl)-2H-[1,4′-bipyridin]-2′-yl)-2-fluorophenyl)-1-methylcyclopropane-1-carboxamide
N-(3-(3-chloro-4-((1S,2S)-2-(4-fluorophenyl)cyclopropyl)-5′,6-dimethyl-2-oxo-2H-[1,4′-
bipyridin]-2′-yl)-2-fluorophenyl)-1-methylcyclopropane-1-carboxamide
N-(3-(4-(2-(1H-indazol-1-yl)cyclopropyl)-3-chloro-5′,6-dimethyl-2-oxo-2H-[1,4′-bipyridin]-
2′-yl)-2-fluorophenyl)-1-methylcyclopropane-1-carboxamide
3-(3,5′-dichloro-4-((1S,2S)-2-(5-fluoropyridin-3-yl)cyclopropyl)-6-methyl-2-oxo-2H-[1,4′-
bipyridin]-2′-yl)-2-fluoro-N-methylbenzamide
3-(3,5′-dichloro-4-((1S,2S)-2-(5-chloropyridin-3-yl)cyclopropyl)-6-methyl-2-oxo-2H-[1,4′-
bipyridin]-2′-yl)-2-fluoro-N-methylbenzamide
N-(3-(3-chloro-3′-fluoro-5′,6-dimethyl-4-((1S,2S)-2-(1-methyl-1H-pyrazol-3-
yl)cyclopropyl)-2-oxo-2H-[1,4′-bipyridin]-2′-yl)-2-fluorophenyl)-1-methylcyclopropane-1-
carboxamide
N-(3-(3-chloro-3′-fluoro-4-((1S,2S)-2-(4-fluorophenyl)cyclopropyl)-5′,6-dimethyl-2-oxo-2H-
[1,4′-bipyridin]-2′-yl)-2-fluorophenyl)-1-methylcyclopropane-1-carboxamide
3-(3-chloro-3′-fluoro-4-((1S,2S)-2-(5-fluoropyridin-3-yl)cyclopropyl)-5′,6-dimethyl-2-oxo-
2H-[1,4′-bipyridin]-2′-yl)-2-fluoro-N,N-dimethylbenzamide
3′-(3-chloro-4-((1S,2S)-2-(4-fluorophenyl)cyclopropyl)-6-methyl-2-oxopyridin-1(2H)-yl)-N-
cyclopropyl-2-fluoro-4′-methyl-[1,1′-biphenyl]-3-carboxamide
N-(3-(3-chloro-4-((1S,2S)-2-(5,6-dihydro-4H-pyrrolo[1,2-b]pyrazol-3-yl)cyclopropyl)-5′,6-
dimethyl-2-oxo-2H-[1,4′-bipyridin]-2′-yl)-2-fluorophenyl)-1-methylcyclopropane-1-carboxamide
3-(3-chloro-5′,6-dimethyl-2-oxo-4-((1S,2S)-2-(pyridin-3-yl)cyclopropyl)-2H-[1,4′-bipyridin]-
2′-yl)-2-fluoro-N,N-dimethylbenzamide
3-(3-chloro-5′,6-dimethyl-2-oxo-4-((1S,2S)-2-(pyridin-4-yl)cyclopropyl)-2H-[1,4′-bipyridin]-
2′-yl)-2-fluoro-N,N-dimethylbenzamide
3-(3-chloro-4-((1S,2S)-2-(5-fluoropyridin-2-yl)cyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(5,6-dihydro-4H-pyrrolo[1,2-b]pyrazol-3-yl)cyclopropyl)-5′,6-
dimethyl-2-oxo-2H-[1,4′-bipyridin]-2′-yl)-2-fluoro-N,N-dimethylbenzamide
3-(3-chloro-4-((1S,2S)-2-(6,7-dihydro-5H-pyrrolo[1,2-a]imidazol-3-yl)cyclopropyl)-5′,6-
dimethyl-2-oxo-2H-[1,4′-bipyridin]-2′-yl)-2-fluoro-N,N-dimethylbenzamide
3-(3-chloro-4-((1S,2S)-2-(2,3-dihydropyrazolo[5,1-b]oxazol-7-yl)cyclopropyl)-5′,6-dimethyl-
2-oxo-2H-[1,4′-bipyridin]-2′-yl)-2-fluoro-N,N-dimethylbenzamide
3-(3-chloro-4-((1S,2S)-2-(6,7-dihydro-5H-pyrazolo[5,1-b][1,3]oxazin-3-yl)cyclopropyl)-5′,6-
dimethyl-2-oxo-2H-[1,4′-bipyridin]-2′-yl)-2-fluoro-N,N-dimethylbenzamide
N-(3-(3-chloro-4-((1S,2S)-2-(5-chloropyridin-3-yl)cyclopropyl)-5′,6-dimethyl-2-oxo-2H-
[1,4′-bipyridin]-2′-yl)-2-fluorophenyl)-1-methylcyclopropane-1-carboxamide
3-(3-chloro-5′,6-dimethyl-2-oxo-4-((1S,2S)-2-(3-oxoisoindolin-4-yl)cyclopropyl)-2H-[1,4′-
bipyridin]-2′-yl)-2-fluoro-N,N-dimethylbenzamide
3-(3-chloro-5′,6-dimethyl-2-oxo-4-((1S,2S)-2-(1-oxoisoindolin-4-yl)cyclopropyl)-2H-[1,4′-
bipyridin]-2′-yl)-2-fluoro-N,N-dimethylbenzamide
N-(3-(3-chloro-4-((1S,2S)-2-(6-chloropyridazin-4-yl)cyclopropyl)-5′,6-dimethyl-2-oxo-2H-
[1,4′-bipyridin]-2′-yl)-2-fluorophenyl)-1-methylcyclopropane-1-carboxamide
N-(3-(3-chloro-4-((1S,2S)-2-(6,7-dihydro-4H-pyrazolo[5,1-c][1,4]oxazin-2-yl)cyclopropyl)-
5′,6-dimethyl-2-oxo-2H-[1,4′-bipyridin]-2′-yl)-2-fluorophenyl)-1-methylcyclopropane-1-carboxamide
N-(3-(3-chloro-5′,6-dimethyl-4-((1S,2S)-2-(1-methyl-6-oxo-1,6-dihydropyridin-3-
yl)cyclopropyl)-2-oxo-2H-[1,4′-bipyridin]-2′-yl)-2-fluorophenyl)-1-methylcyclopropane-1-
carboxamide
N-(3-(3-chloro-4-((1S,2S)-2-(6-chloropyrazin-2-yl)cyclopropyl)-5′,6-dimethyl-2-oxo-2H-
[1,4′-bipyridin]-2′-yl)-2-fluorophenyl)-1-methylcyclopropane-1-carboxamide
3-(3-chloro-4-((1S,2S)-2-(5-chloro-2-methylpyridin-3-yl)cyclopropyl)-5′,6-dimethyl-2-oxo-
2H-[1,4′-bipyridin]-2′-yl)-2-fluoro-N,N-dimethylbenzamide
3-(3-chloro-4-((1S,2S)-2-(5-fluoro-2-methylpyridin-3-yl)cyclopropyl)-5′,6-dimethyl-2-oxo-
2H-[1,4′-bipyridin]-2′-yl)-2-fluoro-N,N-dimethylbenzamide
3-(4-((1S,2S)-2-(benzo[d]thiazol-4-yl)cyclopropyl)-3-chloro-5′,6-dimethyl-2-oxo-2H-[1,4′-
bipyridin]-2′-yl)-2-fluoro-N,N-dimethylbenzamide
3-(4-((1S,2S)-2-(benzo[d]thiazol-7-yl)cyclopropyl)-3-chloro-5′,6-dimethyl-2-oxo-2H-[1,4′-
bipyridin]-2′-yl)-2-fluoro-N,N-dimethylbenzamide
3-(3-chloro-4-((1S,2S)-2-(5-fluoro-6-methylpyridin-2-yl)cyclopropyl)-5′,6-dimethyl-2-oxo-
2H-[1,4′-bipyridin]-2′-yl)-2-fluoro-N,N-dimethylbenzamide
3-(3-chloro-5′,6-dimethyl-4-((1S,2S)-2-(2-methyloxazol-4-yl)cyclopropyl)-2-oxo-2H-[1,4′-
bipyridin]-2′-yl)-2-fluoro-N,N-dimethylbenzamide
3-(3-chloro-4-((1S,2S)-2-(4-fluoro-1-methyl-1H-pyrazol-3-yl)cyclopropyl)-5′,6-dimethyl-2-
oxo-2H-[1,4′-bipyridin]-2′-yl)-2-fluoro-N,N-dimethylbenzamide
N-(3-(3-chloro-4-((1S,2S)-2-(5-chloropyridazin-3-yl)cyclopropyl)-5′,6-dimethyl-2-oxo-2H-
[1,4′-bipyridin]-2′-yl)-2-fluorophenyl)-1-methylcyclopropane-1-carboxamide
3-(3-chloro-4-((1S,2S)-2-(2-(difluoromethyl)-5-fluoropyridin-3-yl)cyclopropyl)-5′,6-
dimethyl-2-oxo-2H-[1,4′-bipyridin]-2′-yl)-2-fluoro-N,N-dimethylbenzamide
3-(3-chloro-4-((1S,2S)-2-(5-fluoropyridin-3-yl)cyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(2-cyano-5-fluoropyridin-3-yl)cyclopropyl)-5′,6-dimethyl-2-oxo-
2H-[1,4′-bipyridin]-2′-yl)-2-fluoro-N,N-dimethylbenzamide
3-(3-chloro-4-((1S,2S)-2-(1-(difluoromethyl)-1H-pyrazol-3-yl)cyclopropyl)-5′,6-dimethyl-2-
oxo-2H-[1,4′-bipyridin]-2′-yl)-2-fluoro-N,N-dimethylbenzamide
3-(3-chloro-4-((1S,2S)-2-(3-(difluoromethyl)phenyl)cyclopropyl)-5′,6-dimethyl-2-oxo-2H-
[1,4′-bipyridin]-2′-yl)-2-fluoro-N,N-dimethylbenzamide
N-(3-(3-chloro-4-((1S,2S)-2-(2-chloropyrimidin-4-yl)cyclopropyl)-5′,6-dimethyl-2-oxo-2H-
[1,4′-bipyridin]-2′-yl)-2-fluorophenyl)-1-methylcyclopropane-1-carboxamide
N-(3-(3-chloro-4-((1S,2S)-2-(6-chloropyrimidin-4-yl)cyclopropyl)-5′,6-dimethyl-2-oxo-2H-
[1,4′-bipyridin]-2′-yl)-2-fluorophenyl)-1-methylcyclopropane-1-carboxamide
N-(3-(3-chloro-5′,6-dimethyl-2-oxo-4-((1S,2S)-2-(pyrazolo[1,5-a]pyridin-3-yl)cyclopropyl)-
2H-[1,4′-bipyridin]-2′-yl)-2-fluorophenyl)-1-methylcyclopropane-1-carboxamide
N-(3-(3-chloro-4-((1S,2S)-2-(5,5-dimethyl-5,6-dihydro-4H-pyrrolo[1,2-b]pyrazol-3-
yl)cyclopropyl)-5′,6-dimethyl-2-oxo-2H-[1,4′-bipyridin]-2′-yl)-2-fluorophenyl)-1-
methylcyclopropane-1-carboxamide
N-(3-(3-chloro-5′,6-dimethyl-4-((1S,2S)-2-(1-methyl-1H-pyrazol-4-yl)cyclopropyl)-2-oxo-
2H-[1,4′-bipyridin]-2′-yl)-2-fluorophenyl)-1-methylcyclopropane-1-carboxamide
N-(3-(3-chloro-4-((1S,2S)-2-(isothiazol-3-yl)cyclopropyl)-5′,6-dimethyl-2-oxo-2H-[1,4′-
bipyridin]-2′-yl)-2-fluorophenyl)-1-methylcyclopropane-1-carboxamide
N-(3-(3-chloro-4-((1S,2S)-2-(5-((dimethyl(oxo)-λ⁶-sulfaneylidene)amino)pyridin-3-
yl)cyclopropyl)-5′,6-dimethyl-2-oxo-2H-[1,4′-bipyridin]-2′-yl)-2-fluorophenyl)-1-
methylcyclopropane-1-carboxamide
N-(3-(3-chloro-4-((1S,2S)-2-(isothiazol-4-yl)cyclopropyl)-5′,6-dimethyl-2-oxo-2H-[1,4′-
bipyridin]-2′-yl)-2-fluorophenyl)-1-methylcyclopropane-1-carboxamide
N-(3-(3-chloro-5′,6-dimethyl-4-((1S,2S)-2-(1-methyl-1H-1,2,3-triazol-4-yl)cyclopropyl)-2-
oxo-2H-[1,4′-bipyridin]-2′-yl)-2-fluorophenyl)-1-methylcyclopropane-1-carboxamide
N-(3-(3-chloro-5′,6-dimethyl-4-((1S,2S)-2-(2-methyl-2H-tetrazol-5-yl)cyclopropyl)-2-oxo-
2H-[1,4′-bipyridin]-2′-yl)-2-fluorophenyl)-1-methylcyclopropane-1-carboxamide
N-(3-(3-chloro-4-((1S,2S)-2-(isothiazol-5-yl)cyclopropyl)-5′,6-dimethyl-2-oxo-2H-[1,4′-
bipyridin]-2′-yl)-2-fluorophenyl)-1-methylcyclopropane-1-carboxamide
N-(3-(3-chloro-5′,6-dimethyl-4-((1S,2S)-2-(2-methyl-2H-1,2,3-triazol-4-yl)cyclopropyl)-2-
oxo-2H-[1,4′-bipyridin]-2′-yl)-2-fluorophenyl)-1-methylcyclopropane-1-carboxamide
N-(3-(4-((1S,2S)-2-(1,2,5-thiadiazol-3-yl)cyclopropyl)-3-chloro-5′,6-dimethyl-2-oxo-2H-
[1,4′-bipyridin]-2′-yl)-2-fluorophenyl)-1-methylcyclopropane-1-carboxamide
N-(3-(3-chloro-5′,6-dimethyl-4-((1S,2S)-2-(1-methyl-1,4,5,6-tetrahydrocyclopenta[c]pyrazol-
3-yl)cyclopropyl)-2-oxo-2H-[1,4′-bipyridin]-2′-yl)-2-fluorophenyl)-1-methylcyclopropane-1-
carboxamide
N-(3-(4-((1S,2S)-2-(benzo[d]thiazol-7-yl)cyclopropyl)-3-chloro-5′,6-dimethyl-2-oxo-2H-
[1,4′-bipyridin]-2′-yl)-2-fluorophenyl)-1-methylcyclopropane-1-carboxamide
N-(3-(3-chloro-5′,6-dimethyl-2-oxo-4-((1S,2S)-2-(1-oxoisoindolin-4-yl)cyclopropyl)-2H-
[1,4′-bipyridin]-2′-yl)-2-fluorophenyl)-1-methylcyclopropane-1-carboxamide
N-(3-(3-chloro-5′,6-dimethyl-2-oxo-4-((1S,2S)-2-(2-oxo-1,2-dihydropyridin-3-
yl)cyclopropyl)-2H-[1,4′-bipyridin]-2′-yl)-2-fluorophenyl)-1-methylcyclopropane-1-carboxamide
N-(3-(3-chloro-5′,6-dimethyl-4-((1S,2S)-2-(2-methylthiazol-4-yl)cyclopropyl)-2-oxo-2H-
[1,4′-bipyridin]-2′-yl)-2-fluorophenyl)-1-methylcyclopropane-1-carboxamide
N-(3-(3-chloro-5′,6-dimethyl-2-oxo-4-((1S,2S)-2-(2-oxoindolin-4-yl)cyclopropyl)-2H-[1,4′-
bipyridin]-2′-yl)-2-fluorophenyl)-1-methylcyclopropane-1-carboxamide
N-(3-(3-chloro-5′,6-dimethyl-2-oxo-4-((1S,2S)-2-(2-oxoindolin-6-yl)cyclopropyl)-2H-[1,4′-
bipyridin]-2′-yl)-2-fluorophenyl)-1-methylcyclopropane-1-carboxamide
3-(3-chloro-4-((1S,2S)-2-(2-chloropyridin-4-yl)cyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(6-chloropyridin-3-yl)cyclopropyl)-5′,6-dimethyl-2-oxo-2H-[1,4′-
bipyridin]-2′-yl)-2-fluoro-N,N-dimethylbenzamide
N-(3-(3-chloro-4-((1S,2S)-2-(6-chloro-3-oxo-2,3-dihydropyridazin-4-yl)cyclopropyl)-5′,6-
dimethyl-2-oxo-2H-[1,4′-bipyridin]-2′-yl)-2-fluorophenyl)-1-methylcyclopropane-1-carboxamide
3-(3-chloro-4-((1S,2S)-2-(2-(difluoromethyl)pyridin-4-yl)cyclopropyl)-5′,6-dimethyl-2-oxo-
2H-[1,4′-bipyridin]-2′-yl)-2-fluoro-N,N-dimethylbenzamide
3-(3-chloro-4-((1S,2S)-2-(2-methoxypyridin-4-yl)cyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(6-fluoropyridin-3-yl)cyclopropyl)-5′,6-dimethyl-2-oxo-2H-[1,4′-
bipyridin]-2′-yl)-2-fluoro-N,N-dimethylbenzamide
3-(3-chloro-4-((1S,2S)-2-(2-fluoropyridin-4-yl)cyclopropyl)-5′,6-dimethyl-2-oxo-2H-[1,4′-
bipyridin]-2′-yl)-2-fluoro-N,N-dimethylbenzamide
3-chloro-2′-(2-fluoro-3-(methylsulfonyl)phenyl)-5′,6-dimethyl-4-((1S,2S)-2-(2-oxo-1,2-
dihydropyridin-3-yl)cyclopropyl)-2H-[1,4′-bipyridin]-2-one
3-chloro-2′-(2-fluoro-3-(methylsulfonyl)phenyl)-5′,6-dimethyl-4-((1S,2S)-2-(5-methyl-2-oxo-
1,2-dihydropyridin-3-yl)cyclopropyl)-2H-[1,4′-bipyridin]-2-one
3-chloro-4-((1S,2S)-2-(5-fluoro-2-oxo-1,2-dihydropyridin-3-yl)cyclopropyl)-2′-(2-fluoro-3-
(methylsulfonyl)phenyl)-5′,6-dimethyl-2H-[1,4′-bipyridin]-2-one
3-chloro-2′-(2-fluoro-3-(methylsulfonyl)phenyl)-5′,6-dimethyl-4-((1S,2S)-2-(2-oxo-5-
(trifluoromethyl)-1,2-dihydropyridin-3-yl)cyclopropyl)-2H-[1,4′-bipyridin]-2-one
3-chloro-3′-fluoro-2′-(2-fluoro-3-(methylsulfonyl)phenyl)-5′,6-dimethyl-4-((1S,2S)-2-(2-oxo-
1,2-dihydropyridin-3-yl)cyclopropyl)-2H-[1,4′-bipyridin]-2-one
3-chloro-3′-fluoro-2′-(2-fluoro-3-(methylsulfonyl)phenyl)-5′,6-dimethyl-4-((1S,2S)-2-(6-oxo-
1,6-dihydropyridin-2-yl)cyclopropyl)-2H-[1,4′-bipyridin]-2-one
3-chloro-4-((1S,2S)-2-(5-(difluoromethyl)pyridin-3-yl)cyclopropyl)-3′-fluoro-2′-(2-fluoro-3-
(methylsulfonyl)phenyl)-5′,6-dimethyl-2H-[1,4′-bipyridin]-2-one
3-chloro-3′-fluoro-2′-(2-fluoro-3-(methylsulfonyl)phenyl)-5′,6-dimethyl-4-((1S,2S)-2-(5-
(trifluoromethyl)pyridin-3-yl)cyclopropyl)-2H-[1,4′-bipyridin]-2-one
3-(3-chloro-4-((1S,2S)-2-(4-fluorophenyl)cyclopropyl)-5′,6-dimethyl-2-oxo-2H-[1,4′-
bipyridin]-2′-yl)-2-fluorobenzamide
3-chloro-2′-(2-fluoro-3-(morpholine-4-carbonyl)phenyl)-4-((1S,2S)-2-(4-
fluorophenyl)cyclopropyl)-5′,6-dimethyl-2H-[1,4′-bipyridin]-2-one
3-(3-chloro-4-((1S,2S)-2-(1-(difluoromethyl)-1H-pyrazol-4-yl)cyclopropyl)-5′,6-dimethyl-2-
oxo-2H-[1,4′-bipyridin]-2′-yl)-2-fluoro-N-methylbenzamide
3-chloro-4-((1S,2S)-2-(1-(difluoromethyl)-1H-pyrazol-4-yl)cyclopropyl)-2′-(2-fluoro-3-
(morpholine-4-carbonyl)phenyl)-5′,6-dimethyl-2H-[1,4′-bipyridin]-2-one
2′-(3-((1S,4S)-2-oxa-5-azabicyclo[2.2.1]heptane-5-carbonyl)-2-fluorophenyl)-3-chloro-4-
((1S,2S)-2-(1-(difluoromethyl)-1H-pyrazol-4-yl)cyclopropyl)-5′,6-dimethyl-2H-[1,4′-bipyridin]-2-one
3-chloro-2′-(3-(3,3-difluoroazetidine-1-carbonyl)-2-fluorophenyl)-4-((1S,2S)-2-(1-
(difluoromethyl)-1H-pyrazol-4-yl)cyclopropyl)-5′,6-dimethyl-2H-[1,4′-bipyridin]-2-one
3-(3-chloro-4-((1S,2S)-2-(1-(difluoromethyl)-1H-pyrazol-4-yl)cyclopropyl)-5′,6-dimethyl-2-
oxo-2H-[1,4′-bipyridin]-2′-yl)-N-cyclopropyl-2-fluorobenzamide
3-(3-chloro-4-((1S,2S)-2-(1-(difluoromethyl)-1H-pyrazol-4-yl)cyclopropyl)-5′,6-dimethyl-2-
oxo-2H-[1,4′-bipyridin]-2′-yl)-2-fluoro-N-(3-hydroxybicyclo[1.1.1]pentan-1-yl)benzamide
6-(3-chloro-4-((1S,2S)-2-(4-fluorophenyl)cyclopropyl)-5′,6-dimethyl-2-oxo-2H-[1,4′-
bipyridin]-2′-yl)-5-fluoro-N-methyl-1H-indazole-4-carboxamide
3-chloro-2′-(4-(3,3-difluoroazetidine-1-carbonyl)-5-fluoro-1H-indazol-6-yl)-4-((1S,2S)-2-(4-
fluorophenyl)cyclopropyl)-5′,6-dimethyl-2H-[1,4′-bipyridin]-2-one
3-chloro-2′-(4-(4,4-difluoropiperidine-1-carbonyl)-5-fluoro-1H-indazol-6-yl)-4-((1S,2S)-2-
(4-fluorophenyl)cyclopropyl)-5′,6-dimethyl-2H-[1,4′-bipyridin]-2-one
2′-(4-((1S,4S)-2-oxa-5-azabicyclo[2.2.1]heptane-5-carbonyl)-5-fluoro-1H-indazol-6-yl)-3-
chloro-4-((1S,2S)-2-(4-fluorophenyl)cyclopropyl)-5′,6-dimethyl-2H-[1,4′-bipyridin]-2-one
3-chloro-2′-(4-((2S,4S)-2,4-dimethylazetidine-1-carbonyl)-5-fluoro-1H-indazol-6-yl)-4-
((1S,2S)-2-(4-fluorophenyl)cyclopropyl)-5′,6-dimethyl-2H-[1,4′-bipyridin]-2-one
3-chloro-2′-(3-(3,3-difluoroazetidine-1-carbonyl)-2-fluorophenyl)-3′-fluoro-4-((1S,2S)-2-(4-
fluorophenyl)cyclopropyl)-5′,6-dimethyl-2H-[1,4′-bipyridin]-2-one
3-chloro-3′-fluoro-2′-(2-fluoro-3-(3-hydroxyazetidine-1-carbonyl)phenyl)-4-((1S,2S)-2-(4-
fluorophenyl)cyclopropyl)-5′,6-dimethyl-2H-[1,4′-bipyridin]-2-one
3-chloro-2′-(3-(3,3-difluoroazetidine-1-carbonyl)-2-fluorophenyl)-4-((1S,2S)-2-(1-
(difluoromethyl)-1H-pyrazol-4-yl)cyclopropyl)-3′-fluoro-5′,6-dimethyl-2H-[1,4′-bipyridin]-2-one
3-chloro-4-((1S,2S)-2-(1-(difluoromethyl)-1H-pyrazol-4-yl)cyclopropyl)-3′-fluoro-2′-(2-
fluoro-3-(3-hydroxyazetidine-1-carbonyl)phenyl)-5′,6-dimethyl-2H-[1,4′-bipyridin]-2-one
3-chloro-3′-fluoro-2′-(2-fluoro-3-(3-(methylsulfonyl)azetidine-1-carbonyl)phenyl)-4-
((1S,2S)-2-(4-fluorophenyl)cyclopropyl)-5′,6-dimethyl-2H-[1,4′-bipyridin]-2-one
3-chloro-3′-fluoro-2′-(2-fluoro-3-(3-(hydroxymethyl)azetidine-1-carbonyl)phenyl)-4-
((1S,2S)-2-(4-fluorophenyl)cyclopropyl)-5′,6-dimethyl-2H-[1,4′-bipyridin]-2-one
3-chloro-3′-fluoro-2′-(2-fluoro-3-(3-(2-hydroxypropan-2-yl)azetidine-1-carbonyl)phenyl)-4-
((1S,2S)-2-(4-fluorophenyl)cyclopropyl)-5′,6-dimethyl-2H-[1,4′-bipyridin]-2-one
1-(3-chloro-4-((1S,2S)-2-(4-fluorophenyl)cyclopropyl)-5′,6-dimethyl-2-oxo-2H-[1,4′-
bipyridin]-2′-yl)-N,N-dimethyl-1H-pyrazole-3-carboxamide
4-(3-chloro-4-((1S,2S)-2-(4-fluorophenyl)cyclopropyl)-5′,6-dimethyl-2-oxo-2H-[1,4′-
bipyridin]-2′-yl)-N,N,1-trimethyl-1H-imidazole-2-carboxamide
4-(3-chloro-4-((1S,2S)-2-(4-fluorophenyl)cyclopropyl)-5′,6-dimethyl-2-oxo-2H-[1,4′-
bipyridin]-2′-yl)-1-methyl-1H-imidazole-2-carboxamide
3-chloro-4-((1S,2S)-2-(5-chloropyridin-3-yl)cyclopropyl)-2′-(3-(3,3-dimethylpyrrolidine-1-
carbonyl)-2-fluorophenyl)-5′,6-dimethyl-2H-[1,4′-bipyridin]-2-one
3-chloro-4-((1S,2S)-2-(5-chloropyridin-3-yl)cyclopropyl)-2′-(2-fluoro-3-((S)-3-hydroxy-3-
methylpyrrolidine-1-carbonyl)phenyl)-5′,6-dimethyl-2H-[1,4′-bipyridin]-2-one
5-(3-chloro-4-((1S,2S)-2-(4-fluorophenyl)cyclopropyl)-5′,6-dimethyl-2-oxo-2H-[1,4′-
bipyridin]-2′-yl)-N,N,1-trimethyl-1H-imidazole-2-carboxamide
3-chloro-4-((1S,2S)-2-(5-chloropyridin-3-yl)cyclopropyl)-2′-(2-fluoro-3-((R)-3-hydroxy-3-
methylpiperidine-1-carbonyl)phenyl)-5′,6-dimethyl-2H-[1,4′-bipyridin]-2-one
3-chloro-4-((1S,2S)-2-(5-chloropyridin-3-yl)cyclopropyl)-2′-(2-fluoro-3-((S)-3-hydroxy-3-
methylpiperidine-1-carbonyl)phenyl)-5′,6-dimethyl-2H-[1,4′-bipyridin]-2-one
2′-(3-(8-azabicyclo[3.2.1]octane-8-carbonyl)-2-fluorophenyl)-3-chloro-4-((1S,2S)-2-(5-
chloropyridin-3-yl)cyclopropyl)-5′,6-dimethyl-2H-[1,4′-bipyridin]-2-one
3-chloro-2′-(3-(3,3-difluoropyrrolidine-1-carbonyl)-2-fluorophenyl)-3′-fluoro-4-((1S,2S)-2-
(4-fluorophenyl)cyclopropyl)-5′,6-dimethyl-2H-[1,4′-bipyridin]-2-one
3-chloro-3′-fluoro-2′-(2-fluoro-3-((R)-3-hydroxypyrrolidine-1-carbonyl)phenyl)-4-((1S,2S)-
2-(4-fluorophenyl)cyclopropyl)-5′,6-dimethyl-2H-[1,4′-bipyridin]-2-one
3-chloro-3′-fluoro-2′-(2-fluoro-3-((S)-3-hydroxypyrrolidine-1-carbonyl)phenyl)-4-((1S,2S)-2-
(4-fluorophenyl)cyclopropyl)-5′,6-dimethyl-2H-[1,4′-bipyridin]-2-one
4-(3-chloro-4-((1S,2S)-2-(5-chloropyridin-3-yl)cyclopropyl)-5′,6-dimethyl-2-oxo-2H-[1,4′-
bipyridin]-2′-yl)-3-fluoro-N,N-dimethylthiophene-2-carboxamide
3-(3-chloro-4-((1S,2S)-2-(5-fluoropyridin-3-yl)cyclopropyl)-5′,6-dimethyl-2-oxo-2H-[1,4′-
bipyridin]-2′-yl)-2-fluoro-N-methylbenzamide
1-(3-(3-chloro-3′-fluoro-4-((1S,2S)-2-(4-fluorophenyl)cyclopropyl)-5′,6-dimethyl-2-oxo-2H-
[1,4′-bipyridin]-2′-yl)-2-fluorobenzoyl)azetidine-3-carbonitrile
(S)-1-(3-(3-chloro-3′-fluoro-4-((1S,2S)-2-(4-fluorophenyl)cyclopropyl)-5′,6-dimethyl-2-oxo-
2H-[1,4′-bipyridin]-2′-yl)-2-fluorobenzoyl)pyrrolidine-3-carbonitrile
(R)-1-(3-(3-chloro-3′-fluoro-4-((1S,2S)-2-(4-fluorophenyl)cyclopropyl)-5′,6-dimethyl-2-oxo-
2H-[1,4′-bipyridin]-2′-yl)-2-fluorobenzoyl)pyrrolidine-3-carbonitrile
3-chloro-4-((1S,2S)-2-(5-chloropyridin-3-yl)cyclopropyl)-3′-fluoro-2′-(2-fluoro-3-(3-
hydroxyazetidine-1-carbonyl)phenyl)-5′,6-dimethyl-2H-[1,4′-bipyridin]-2-one
3-chloro-4-((1S,2S)-2-(5-chloropyridin-3-yl)cyclopropyl)-3′-fluoro-2′-(2-fluoro-3-(3-
hydroxy-3-methylazetidine-1-carbonyl)phenyl)-5′,6-dimethyl-2H-[1,4′-bipyridin]-2-one
3-chloro-4-((1S,2S)-2-(5-chloropyridin-3-yl)cyclopropyl)-3′-fluoro-2′-(2-fluoro-3-(3-
(hydroxymethyl)azetidine-1-carbonyl)phenyl)-5′,6-dimethyl-2H-[1,4′-bipyridin]-2-one
3-(3-chloro-4-((1S,2S)-2-(5-fluoropyridin-3-yl)cyclopropyl)-5′,6-dimethyl-2-oxo-2H-[1,4′-
bipyridin]-2′-yl)-2-fluoro-N,N-bis(methyl-d3)benzamide
3-(3-chloro-4-((1S,2S)-2-(5-chloropyridin-3-yl)cyclopropyl)-5′,6-dimethyl-2-oxo-2H-[1,4′-
bipyridin]-2′-yl)-2-fluoro-N,N-bis(methyl-d3)benzamide
5-(3-chloro-4-((1S,2S)-2-(5-chloropyridin-3-yl)cyclopropyl)-5′,6-dimethyl-2-oxo-2H-[1,4′-
bipyridin]-2′-yl)-4-fluoro-N,N-dimethylthiophene-3-carboxamide
3-(3-chloro-4-((1S,2S)-2-(5-chloropyridin-3-yl)cyclopropyl)-3′-fluoro-5′,6-dimethyl-2-oxo-
2H-[1,4′-bipyridin]-2′-yl)-N-cyclopropyl-2-fluorobenzamide
3-(3-chloro-4-((1S,2S)-2-(5-chloropyridin-3-yl)cyclopropyl)-3′-fluoro-5′,6-dimethyl-2-oxo-
2H-[1,4′-bipyridin]-2′-yl)-2-fluoro-N-methylbenzamide
N-(1-(3-(3-chloro-3′-fluoro-4-((1S,2S)-2-(4-fluorophenyl)cyclopropyl)-5′,6-dimethyl-2-oxo-
2H-[1,4′-bipyridin]-2′-yl)-2-fluorobenzoyl)azetidin-3-yl)methanesulfonamide
1-(1-(3-(3-chloro-3′-fluoro-4-((1S,2S)-2-(4-fluorophenyl)cyclopropyl)-5′,6-dimethyl-2-oxo-
2H-[1,4′-bipyridin]-2′-yl)-2-fluorobenzoyl)azetidin-3-yl)-3-methylurea
3-(3-chloro-4-((1S,2S)-2-(5-fluoropyridin-3-yl)cyclopropyl)-5′,6-dimethyl-2-oxo-2H-[1,4′-
bipyridin]-2′-yl)-2-fluoro-N-(methyl-d3)benzamide
3-(3-chloro-4-((1S,2S)-2-(5-fluoropyridin-3-yl)cyclopropyl)-5′,6-dimethyl-2-oxo-2H-[1,4′-
bipyridin]-2′-yl)-N-cyclopropyl-2-fluoro-N-methylbenzamide
3-(3-chloro-3′-fluoro-4-((1S,2S)-2-(5-fluoropyridin-3-yl)cyclopropyl)-5′,6-dimethyl-2-oxo-
2H-[1,4′-bipyridin]-2′-yl)-N-cyclopropyl-2-fluoro-N-(methyl-d3)benzamide
3-chloro-4-((1S,2S)-2-(5-(difluoromethyl)pyridin-3-yl)cyclopropyl)-3′-fluoro-2′-(2-fluoro-3-
(3-hydroxyazetidine-1-carbonyl)phenyl)-5′,6-dimethyl-2H-[1,4′-bipyridin]-2-one
3-(3-chloro-4-((1S,2S)-2-(5-(difluoromethyl)pyridin-3-yl)cyclopropyl)-3′-fluoro-5′,6-
dimethyl-2-oxo-2H-[1,4′-bipyridin]-2′-yl)-2-fluoro-N-methylbenzamide
3-chloro-3′-fluoro-2′-(2-fluoro-3-(3-(oxetan-3-yl)azetidine-1-carbonyl)phenyl)-4-((1S,2S)-2-
(4-fluorophenyl)cyclopropyl)-5′,6-dimethyl-2H-[1,4′-bipyridin]-2-one
3-chloro-2′-(3-(3-(difluoromethoxy)azetidine-1-carbonyl)-2-fluorophenyl)-3′-fluoro-4-
((1S,2S)-2-(4-fluorophenyl)cyclopropyl)-5′,6-dimethyl-2H-[1,4′-bipyridin]-2-one
3-chloro-4-((1S,2S)-2-(5-chloropyridin-3-yl)cyclopropyl)-3′-fluoro-2′-(2-fluoro-3-((S)-2-
methylazetidine-1-carbonyl)phenyl)-5′,6-dimethyl-2H-[1,4′-bipyridin]-2-one
3-chloro-4-((1S,2S)-2-(5-chloropyridin-3-yl)cyclopropyl)-2′-(3-((2S,4S)-2,4-
dimethylazetidine-1-carbonyl)-2-fluorophenyl)-3′-fluoro-5′,6-dimethyl-2H-[1,4′-bipyridin]-2-one
3-chloro-4-((1S,2S)-2-(5-chloropyridin-3-yl)cyclopropyl)-3′-fluoro-2′-(2-fluoro-3-(4-
azaspiro[2.3]hexane-4-carbonyl)phenyl)-5′,6-dimethyl-2H-[1,4′-bipyridin]-2-one
3-chloro-4-((1S,2S)-2-(5-chloropyridin-3-yl)cyclopropyl)-3′-fluoro-2′-(2-fluoro-3-(1,6-
diazaspiro[3.3]heptane-1-carbonyl)phenyl)-5′,6-dimethyl-2H-[1,4′-bipyridin]-2-one
3-chloro-4-((1S,2S)-2-(5-chloropyridin-3-yl)cyclopropyl)-3′-fluoro-2′-(2-fluoro-3-((R)-2-
(trifluoromethyl)azetidine-1-carbonyl)phenyl)-5′,6-dimethyl-2H-[1,4′-bipyridin]-2-one
3-(3-chloro-4-((1S,2S)-2-(5-chloropyridin-3-yl)cyclopropyl)-3′-fluoro-5′,6-dimethyl-2-oxo-
2H-[1,4′-bipyridin]-2′-yl)-2-fluoro-N-(2-hydroxyethyl)benzamide
3-(3-chloro-4-((1S,2S)-2-(5-chloropyridin-3-yl)cyclopropyl)-3′-fluoro-5′,6-dimethyl-2-oxo-
2H-[1,4′-bipyridin]-2′-yl)-2-fluoro-N-(methyl-d3)benzamide
3-(3-chloro-4-((1S,2S)-2-(5-chloropyridin-3-yl)cyclopropyl)-3′-fluoro-5′,6-dimethyl-2-oxo-
2H-[1,4′-bipyridin]-2′-yl)-2-fluoro-N-(3-hydroxycyclobutyl)benzamide
3-(3-chloro-4-((1S,2S)-2-(5-chloropyridin-3-yl)cyclopropyl)-3′-fluoro-5′,6-dimethyl-2-oxo-
2H-[1,4′-bipyridin]-2′-yl)-2-fluoro-N-(3-fluorocyclobutyl)benzamide
6-(3-chloro-4-((1S,2S)-2-(4-fluorophenyl)cyclopropyl)-5′,6-dimethyl-2-oxo-2H-[1,4′-
bipyridin]-2′-yl)-7-fluorobenzo[d]oxazol-2(3H)-one
2′-(3-amino-2-fluorophenyl)-3-chloro-4-((1S,2S)-2-(4-fluorophenyl)cyclopropyl)-5′,6-
dimethyl-2H-[1,4′-bipyridin]-2-one
N-(3-(3-chloro-4-((1S,2S)-2-(4-fluorophenyl)cyclopropyl)-5′,6-dimethyl-2-oxo-2H-[1,4′-
bipyridin]-2′-yl)-2-fluorophenyl)acetamide
3-(3-chloro-4-((1S,2S)-2-(1-(difluoromethyl)-1H-pyrazol-4-yl)cyclopropyl)-5′,6-dimethyl-2-
oxo-2H-[1,4′-bipyridin]-2′-yl)-2-fluoro-N,N-dimethylbenzamide
N-(3-(3-chloro-4-((1S,2S)-2-(1-(difluoromethyl)-1H-pyrazol-4-yl)cyclopropyl)-5′,6-
dimethyl-2-oxo-2H-[1,4′-bipyridin]-2′-yl)phenyl)methanesulfonamide
3-chloro-2′-(3-((dimethyl(oxo)-λ⁶-sulfaneylidene)amino)phenyl)-4-((1S,2S)-2-(4-
fluorophenyl)cyclopropyl)-5′,6-dimethyl-2H-[1,4′-bipyridin]-2-one
3-chloro-3″-fluoro-4-((1S,2S)-2-(4-fluorophenyl)cyclopropyl)-2″-(3-hydroxy-3-
methylazetidin-1-yl)-5′,6-dimethyl-2H-[1,4′:2′,4″-terpyridin]-2-one
3-chloro-5″-((dimethyl(oxo)-λ⁶-sulfaneylidene)amino)-4-((1S,2S)-2-(4-
fluorophenyl)cyclopropyl)-5′,6-dimethyl-2H-[1,4′:2′,3″-terpyridin]-2-one
3-chloro-4-((1S,2S)-2-(1-(difluoromethyl)-1H-pyrazol-4-yl)cyclopropyl)-2′-(5-fluoro-1H-
indazol-6-yl)-5′,6-dimethyl-2H-[1,4′-bipyridin]-2-one
3-(3-chloro-4-((1S,2S)-2-(1-(difluoromethyl)-1H-pyrazol-4-yl)cyclopropyl)-5′,6-dimethyl-2-
oxo-2H-[1,4′-bipyridin]-2′-yl)-2-fluorobenzamide
6-(3-chloro-4-((1S,2S)-2-(4-fluorophenyl)cyclopropyl)-5′,6-dimethyl-2-oxo-2H-[1,4′-
bipyridin]-2′-yl)-5-fluoro-3-hydroxy-3-methylindolin-2-one
3-(3-chloro-3′-fluoro-4-((1S,2S)-2-(4-fluorophenyl)cyclopropyl)-5′,6-dimethyl-2-oxo-2H-
[1,4′-bipyridin]-2′-yl)-2-fluorobenzamide
3-(3-chloro-4-((1S,2S)-2-(1-(difluoromethyl)-1H-pyrazol-4-yl)cyclopropyl)-3′-fluoro-5′,6-
dimethyl-2-oxo-2H-[1,4′-bipyridin]-2′-yl)-2-fluorobenzamide
3-(3-chloro-4-((1S,2S)-2-(1-(difluoromethyl)-1H-pyrazol-4-yl)cyclopropyl)-3′-fluoro-5′,6-
dimethyl-2-oxo-2H-[1,4′-bipyridin]-2′-yl)-2-fluoro-N,N-dimethylbenzamide
3-(3-chloro-3′-fluoro-4-((1S,2S)-2-(4-fluorophenyl)cyclopropyl)-5′,6-dimethyl-2-oxo-2H-
[1,4′-bipyridin]-2′-yl)-2-fluoro-N,N-dimethylbenzamide
6-(3-chloro-4-((1S,2S)-2-(4-fluorophenyl)cyclopropyl)-5′,6-dimethyl-2-oxo-2H-[1,4′-
bipyridin]-2′-yl)-3-hydroxy-3-isopropyl-4-(trifluoromethyl)indolin-2-one
(S)-6-(3-chloro-4-((1S,2S)-2-(4-fluorophenyl)cyclopropyl)-5′,6-dimethyl-2-oxo-2H-[1,4′-
bipyridin]-2′-yl)-5-fluoro-3-hydroxy-3-isopropylindolin-2-one
(R)-6-(3-chloro-4-((1S,2S)-2-(4-fluorophenyl)cyclopropyl)-5′,6-dimethyl-2-oxo-2H-[1,4′-
bipyridin]-2′-yl)-5-fluoro-3-hydroxy-3-isopropylindolin-2-one
3-chloro-3″-fluoro-4-((1S,2S)-2-(4-fluorophenyl)cyclopropyl)-5′,6-dimethyl-2-oxo-2H-
[1,4′:2′,4″-terpyridine]-2″-carbonitrile
3-(3-chloro-3′-fluoro-4-((1S,2S)-2-(4-fluorophenyl)cyclopropyl)-5′,6-dimethyl-2-oxo-2H-
[1,4′-bipyridin]-2′-yl)-2-fluoro-N,N-dimethylbenzamide
5-(3-chloro-4-((1S,2S)-2-(4-fluorophenyl)cyclopropyl)-5′,6-dimethyl-2-oxo-2H-[1,4′-
bipyridin]-2′-yl)-4-fluoro-3,3-dimethylindolin-2-one
4-(3-chloro-4-((1S,2S)-2-(4-fluorophenyl)cyclopropyl)-5′,6-dimethyl-2-oxo-2H-[1,4′-
bipyridin]-2′-yl)-3-fluoro-N,N-dimethylbenzamide
4-(3-chloro-4-((1S,2S)-2-(4-fluorophenyl)cyclopropyl)-5′,6-dimethyl-2-oxo-2H-[1,4′-
bipyridin]-2′-yl)-3-fluorobenzamide
6-(3-chloro-4-((1S,2S)-2-(4-fluorophenyl)cyclopropyl)-5′,6-dimethyl-2-oxo-2H-[1,4′-
bipyridin]-2′-yl)-3-hydroxy-3-methylindolin-2-one
3-chloro-2′-(2-fluoro-3-(methylsulfonyl)phenyl)-4-((1S,2S)-2-(4-fluorophenyl)cyclopropyl)-
5′,6-dimethyl-2H-[1,4′-bipyridin]-2-onea
6-(3-chloro-4-((1S,2S)-2-(4-fluorophenyl)cyclopropyl)-5′,6-dimethyl-2-oxo-2H-[1,4′-
bipyridin]-2′-yl)-3,3-difluoroindolin-2-one
3-chloro-2′-(2-fluoro-3-(methylsulfonyl)phenyl)-4-((1S,2S)-2-(5-fluoropyridin-3-
yl)cyclopropyl)-5′,6-dimethyl-2H-[1,4′-bipyridin]-2-one
3-chloro-4-((1S,2S)-2-(5-chloropyridin-3-yl)cyclopropyl)-2′-(2-fluoro-3-
(methylsulfonyl)phenyl)-5′,6-dimethyl-2H-[1,4′-bipyridin]-2-one
3-(3-chloro-4-((1S,2S)-2-(5-fluoropyridin-3-yl)cyclopropyl)-5′,6-dimethyl-2-oxo-2H-[1,4′-
bipyridin]-2′-yl)-2-fluorobenzamide
N-(3-(3-chloro-4-((1S,2S)-2-(4-fluorophenyl)cyclopropyl)-5′,6-dimethyl-2-oxo-2H-[1,4′-
bipyridin]-2′-yl)-2-fluorophenyl)cyclopropanecarboxamide
N-(3-(3-chloro-4-((1S,2S)-2-(4-fluorophenyl)cyclopropyl)-5′,6-dimethyl-2-oxo-2H-[1,4′-
bipyridin]-2′-yl)-2-fluorophenyl)-2-hydroxy-2-methylpropanamide
N-(3-(3-chloro-4-((1S,2S)-2-(5-chloropyridin-3-yl)cyclopropyl)-5′,6-dimethyl-2-oxo-2H-
[1,4′-bipyridin]-2′-yl)-2-fluorophenyl)-2-hydroxy-2-methylpropanamide
3-chloro-3′-fluoro-2′-(2-fluoro-3-(methylsulfonyl)phenyl)-4-((1S,2S)-2-(5-fluoropyridin-3-
yl)cyclopropyl)-5′,6-dimethyl-2H-[1,4′-bipyridin]-2-one
3-(3-chloro-3′-fluoro-4-((1S,2S)-2-(5-fluoropyridin-3-yl)cyclopropyl)-5′,6-dimethyl-2-oxo-
2H-[1,4′-bipyridin]-2′-yl)-2-fluoro-N-methylbenzamide
3-(3-chloro-3′-fluoro-4-((1S,2S)-2-(5-fluoropyridin-3-yl)cyclopropyl)-5′,6-dimethyl-2-oxo-
2H-[1,4′-bipyridin]-2′-yl)-N-cyclopropyl-2-fluorobenzamide
3-chloro-4-((1S,2S)-2-(5-chloropyridin-3-yl)cyclopropyl)-3′-fluoro-2′-(2-fluoro-3-
(methylsulfonyl)phenyl)-5′,6-dimethyl-2H-[1,4′-bipyridin]-2-one
3-(3-chloro-3′-fluoro-4-((1S,2S)-2-(5-fluoropyridin-3-yl)cyclopropyl)-5′,6-dimethyl-2-oxo-
2H-[1,4′-bipyridin]-2′-yl)-2-fluorobenzamide
3-(3-chloro-4-((1S,2S)-2-(5-chloropyridin-3-yl)cyclopropyl)-3′-fluoro-5′,6-dimethyl-2-oxo-
2H-[1,4′-bipyridin]-2′-yl)-2-fluorobenzamide
3-chloro-4-((1S,2S)-2-(5-chloropyridin-3-yl)cyclopropyl)-3′-fluoro-2′-(2-fluoro-3-(S-
methylsulfonimidoyl)phenyl)-5′,6-dimethyl-2H-[1,4′-bipyridin]-2-one
3-chloro-4-((1S,2S)-2-(5-chloropyridin-3-yl)cyclopropyl)-2′-(2-fluoro-3-(4-methylthiazol-5-
yl)phenyl)-5′,6-dimethyl-2H-[1,4′-bipyridin]-2-one
3-chloro-4-((1S,2S)-2-(5-chloropyridin-3-yl)cyclopropyl)-2′-(2-fluoro-3-(3-methylisothiazol-
4-yl)phenyl)-5′,6-dimethyl-2H-[1,4′-bipyridin]-2-one
3-chloro-4-((1S,2S)-2-(5-chloropyridin-3-yl)cyclopropyl)-2′-(2-fluoro-3-(1-methyl-1H-1,2,4-
triazol-5-yl)phenyl)-5′,6-dimethyl-2H-[1,4′-bipyridin]-2-one
N-(3-(3-chloro-4-((1S,2S)-2-(4-fluorophenyl)cyclopropyl)-5′,6-dimethyl-2-oxo-2H-[1,4′-
bipyridin]-2′-yl)-2-fluorophenyl)-3-methyloxetane-3-carboxamide
N-(3-(3-chloro-4-((1S,2S)-2-(4-fluorophenyl)cyclopropyl)-5′,6-dimethyl-2-oxo-2H-[1,4′-
bipyridin]-2′-yl)-2-fluorophenyl)oxetane-3-carboxamide
N-(3-(3-chloro-4-((1S,2S)-2-(4-fluorophenyl)cyclopropyl)-5′,6-dimethyl-2-oxo-2H-[1,4′-
bipyridin]-2′-yl)-2-fluorophenyl)-1-(difluoromethyl)cyclopropane-1-carboxamide
N-(3-(3-chloro-4-((1S,2S)-2-(4-fluorophenyl)cyclopropyl)-5′,6-dimethyl-2-oxo-2H-[1,4′-
bipyridin]-2′-yl)-2-fluorophenyl)-1-methoxycyclobutane-1-carboxamide
N-(3-(3-chloro-4-((1S,2S)-2-(4-fluorophenyl)cyclopropyl)-5′,6-dimethyl-2-oxo-2H-[1,4′-
bipyridin]-2′-yl)-2-fluorophenyl)-1-fluorocyclopropane-1-carboxamide
N-(3-(3-chloro-4-((1S,2S)-2-(5-fluoropyridin-3-yl)cyclopropyl)-5′,6-dimethyl-2-oxo-2H-
[1,4′-bipyridin]-2′-yl)-2-fluorophenyl)-1-methylcyclopropane-1-carboxamide
3-(3-chloro-4-((1S,2S)-2-(cyclopropylcarbamoyl)cyclopropyl)-5′,6-dimethyl-2-oxo-2H-[1,4′-
bipyridin]-2′-yl)-2-fluoro-N,N-dimethylbenzamide
3-(3-chloro-5′,6-dimethyl-2-oxo-4-((1S,2S)-2-(phenylcarbamoyl)cyclopropyl)-2H-[1,4′-
bipyridin]-2′-yl)-2-fluoro-N,N-dimethylbenzamide
3-(3-chloro-4-((1S,2S)-2-(1,1-dioxidothiomorpholine-4-carbonyl)cyclopropyl)-5′,6-dimethyl-
2-oxo-2H-[1,4′-bipyridin]-2′-yl)-2-fluoro-N,N-dimethylbenzamide
2″-(3-(aminomethyl)-3-hydroxyazetidin-1-yl)-3-chloro-3″-fluoro-4-((1S,2S)-2-(4-
fluorophenyl)cyclopropyl)-5′,6-dimethyl-2H-[1,4′:2′,4″-terpyridin]-2-one
2″-(3-amino-3-methylazetidin-1-yl)-3-chloro-3″-fluoro-4-((1S,2S)-2-(4-
fluorophenyl)cyclopropyl)-5′,6-dimethyl-2H-[1,4′:2′,4″-terpyridin]-2-one
3-chloro-3″-fluoro-4-((1S,2S)-2-(5-fluoropyridin-3-yl)cyclopropyl)-2″-(3-hydroxy-3-
methylazetidin-1-yl)-5′,6-dimethyl-2H-[1,4′:2′,4″-terpyridin]-2-one
2″-(3-amino-3-methylazetidin-1-yl)-3-chloro-3″-fluoro-4-((1S,2S)-2-(5-fluoropyridin-3-
yl)cyclopropyl)-5′,6-dimethyl-2H-[1,4′:2′,4″-terpyridin]-2-one
N-(3-chloro-3″-fluoro-4-((1S,2S)-2-(4-fluorophenyl)cyclopropyl)-5′,6-dimethyl-2-oxo-2H-
[1,4′:2′,4″-terpyridin]-2″-yl)-1-methylcyclopropane-1-carboxamide
2-fluoro-3-(4-((1S,2S)-2-(4-fluorophenyl)cyclopropyl)-5′,6-dimethyl-2-oxo-2H-[1,4′-
bipyridin]-2′-yl)-N,N-dimethylbenzamide
3-(3-bromo-4-((1S,2S)-2-(4-fluorophenyl)cyclopropyl)-5′,6-dimethyl-2-oxo-2H-[1,4′-
bipyridin]-2′-yl)-2-fluoro-N,N-dimethylbenzamide
methyl (3-(3-chloro-4-((1S,2S)-2-(5-chloropyridin-3-yl)cyclopropyl)-5′,6-dimethyl-2-oxo-
2H-[1,4′-bipyridin]-2′-yl)-2-fluorophenyl)carbamate
3-(3-(3-chloro-4-((1S,2S)-2-(5-chloropyridin-3-yl)cyclopropyl)-5′,6-dimethyl-2-oxo-2H-
[1,4′-bipyridin]-2′-yl)-2-fluorophenyl)-1,1-dimethylurea
3-(3-(3-chloro-4-((1S,2S)-2-(5-chloropyridin-3-yl)cyclopropyl)-5′,6-dimethyl-2-oxo-2H-
[1,4′-bipyridin]-2′-yl)-2-fluorophenyl)oxazolidin-2-one
N-(3-(3-chloro-4-((1S,2S)-2-(5-chloropyridin-3-yl)cyclopropyl)-5′,6-dimethyl-2-oxo-2H-
[1,4′-bipyridin]-2′-yl)-2-fluorophenyl)-N,1-dimethylcyclopropane-1-carboxamide
3-(3-chloro-4-((1S,2S)-2-(3-(cyclopropylcarbamoyl)-5-fluorophenyl)cyclopropyl)-5′,6-
dimethyl-2-oxo-2H-[1,4′-bipyridin]-2′-yl)-2-fluoro-N,N-dimethylbenzamide
3-chloro-5-((1S,2S)-2-(3-chloro-3′-fluoro-2′-(2-fluoro-3-((R)-2-(trifluoromethyl)azetidine-1-
carbonyl)phenyl)-5′,6-dimethyl-2-oxo-2H-[1,4′-bipyridin]-4-yl)cyclopropyl)pyridine 1-oxide
3-((1S,2S)-2-(3-chloro-3′-fluoro-2′-(2-fluoro-3-(methylsulfonyl)phenyl)-5′,6-dimethyl-2-oxo-
2H-[1,4′-bipyridin]-4-yl)cyclopropyl)-5-fluoropyridine 1-oxide
3-chloro-5-((1S,2S)-2-(3-chloro-3′-fluoro-2′-(2-fluoro-3-(methylsulfonyl)phenyl)-5′,6-
dimethyl-2-oxo-2H-[1,4′-bipyridin]-4-yl)cyclopropyl)pyridine 1-oxide
3-((1S,2S)-2-(3-chloro-3′-fluoro-2′-(2-fluoro-3-(methylsulfonyl)phenyl)-5′,6-dimethyl-2-oxo-
2H-[1,4′-bipyridin]-4-yl)cyclopropyl)-5-(difluoromethyl)pyridine 1 -oxide
3-((1S,2S)-2-(3-chloro-3′-fluoro-2′-(2-fluoro-3-(methylsulfonyl)phenyl)-5′,6-dimethyl-2-oxo-
2H-[1,4′-bipyridin]-4-yl)cyclopropyl)-5-(trifluoromethyl)pyridine 1 -oxide

or a pharmaceutically acceptable salt thereof.

Clause 140. A pharmaceutical composition comprising the compound of any one of clauses 1 to 139, or a pharmaceutically acceptable salt thereof, and a pharmaceutically acceptable excipient or carrier.

Clause 141. The pharmaceutical composition of clause 140, further comprising one or more additional therapeutic agents, or a pharmaceutically acceptable salt thereof.

Clause 142. A method of inhibiting p38 MAP kinase activity in a subject in need thereof, comprising administering to the subject a therapeutically effective amount of the compound of any one of clauses 1 to 139, or a pharmaceutically acceptable salt thereof, or a therapeutically effective amount of the pharmaceutical composition of clause 140 or 141.

Clause 143. A method of inhibiting MK2 activity in a subject in need thereof, comprising administering to the subject a therapeutically effective amount of the compound of any one of clauses 1 to 139, or a pharmaceutically acceptable salt thereof, or a therapeutically effective amount of the pharmaceutical composition of clause 140 or 141.

Clause 144. A method of treating a p38 MAP kinase-mediated disease or disorder in a subject in need thereof, comprising administering to the subject a therapeutically effective amount of the compound of any one of clauses 1 to 139, or a pharmaceutically acceptable salt thereof, or a therapeutically effective amount of the pharmaceutical composition of clause 140 or 141.

Clause 145. A method of treating an MK2-mediated disease or disorder in a subject in need thereof, comprising administering to the subject a therapeutically effective amount of the compound of any one of clauses 1 to 139, or a pharmaceutically acceptable salt thereof, or a therapeutically effective amount of the pharmaceutical composition of clause 140 or 141.

Clause 146. A method of treating an autoimmune disorder, a chronic inflammatory disorder, an acute inflammatory disorder, an auto-inflammatory disorder, a fibrotic disorder, a metabolic disorder, a neoplastic disorder, a cardiovascular disorder, or a cerebrovascular disorder in a subject in need thereof, comprising administering to the subject a therapeutically effective amount of the compound of any one of clauses 1 to 139, or a pharmaceutically acceptable salt thereof, or a therapeutically effective amount of the pharmaceutical composition of clause 140 or 141.

Clause 147. A method of treating an inflammatory condition in a subject in need thereof, comprising administering to the subject a therapeutically effective amount of the compound of any one of clauses 1 to 139, or a pharmaceutically acceptable salt thereof, or a therapeutically effective amount of the pharmaceutical composition of clause 140 or 141.

Clause 148. The method of clause 147, wherein the inflammatory condition is selected from inflammatory bowel disease, psoriasis, psoriatic arthritis, rheumatoid arthritis, glomerulonephritis, mixed connective tissue disease (MCTD), dermatomyositis, polymyositis, systemic sclerosis, antineutrophil cytoplasmic antibody-associated vasculitis, anti-phospholipid syndrome, autoimmune hemolytic anemia, macrophage activation syndrome driven inflammatory anemia, IgA nephropathy, type I diabetes, non-alcoholic steatohepatitis, and Sjogren's syndrome.

Clause 149. A method of treating rheumatoid arthritis in a subject in need thereof, comprising administering to the subject a therapeutically effective amount of the compound of any one of clauses 1 to 139, or a pharmaceutically acceptable salt thereof, or a therapeutically effective amount of the pharmaceutical composition of clause 140 or 141.

Clause 150. The method of any one of clauses 142 to 149, further comprising administering a therapeutically effective amount of one or more additional therapeutic agents, or a pharmaceutically acceptable salt thereof.

Clause 151. The method of clause 150, wherein the one or more additional therapeutic agents is selected from the group consisting of veltuzumab, PF-06835375, eculizumab, milatuzumab, SM-06, SM-03, BT-063, QX-006-N, BOS-161721, AK-101, TNX-1500, theralizumab, daxdilimab, TAK-079, felzartamab, itolizumab, anifrolumab, iscalimab, dapirolizumab pegol, lanalumab, LY-3361237, JNJ-55920839, UBP-1213, DS-7011, PFI-102, BIIB-059, obexelimab, talacotuzumab, vobarilizumab, TE-2324, PRV-3279, chloroquine, hydroxychloroquine, hydroxychloroquine sulfate, COV-08-0064; GNKS-356, AVO-101, rozibafusp alfa, VRN-02, annexuzlimab, ALPN-101, bendamustine hydrochloride, BMS-986256, NKTR-35, atacicept, telitacicept, BMS-986256, M-5049, KZR-616, KPG-818, verdinexor, ALPN-303, valziflocept, LA-1, cenerimod, prednisone, corticotropin, deucravacitinib, CPL-409116, CS-12192, tofacitinib citrate, ISB-830, DV-1079, julemic acid, iberdomide, TAM-01, BML-258, brepocitinib, SDC-1801, SDC-1802, ICP-330, NTR-441, dalazatide, GSK-2646264, SKI-O-703, lanraplenib (GS-9876), GNS-1653, HMPL-523, RSLV-132, interleukin-2 follow-on biologic, interleukin-2 Anteluke, interking recombinant human interleukin-2, ILT-101, CUG-252, DZ-2002, PEGylated HLA-x (SLE), AC-0058, fenebrutinib, XNW-1011, tirabrutinib hydrochloride, branebrutinib, elsubrutinib, orelabrutinib, DWP-213388, INV-103, R-salbutamol sulphate, anchorins, NIK-SMI1, X-6, INV-17, Oshadi D, baricitinib, upadacitinib, filgotinib, itacitinib, INCB-54707, delgocitinib, DWP-212525, CKD-971, as mometasone, betamethasone, forigerimod, anandamide, DCB-SLE1, arsenic trioxide, tairuimide, TV-4710 (edratide), allogeneic human umbilical cord-derived mesenchymal stem cell therapy (hUC-MSCs), LC-200, BI-705564, SM-934, GX-101, TXR-712, TXR-711, CIT-013, MHV-370, Panzyga®, TPX-6001, TPX-7001, artenimol, and AMG-592, or a pharmaceutically acceptable salt of any of the foregoing, or any combination thereof.

Clause 152. The method of any one of clauses 142 to 151, wherein the subject is a human.

Clause 153. A compound of any one of clauses 1 to 139, or a pharmaceutically acceptable salt thereof, or a pharmaceutical composition of clause 140 or 141 for use in therapy.

Clause 154. A compound of any one of clauses 1 to 139, or a pharmaceutically acceptable salt thereof, or a pharmaceutical composition of clause 140 or 141 for use in a method of inhibiting p38 MAP kinase activity in a subject in need thereof, comprising administering to the subject a therapeutically effective amount of the compound, or a pharmaceutically acceptable salt thereof, or a therapeutically effective amount of the pharmaceutical composition.

Clause 155. A compound of any one of clauses 1 to 139, or a pharmaceutically acceptable salt thereof, or a pharmaceutical composition of clause 140 or 141 for use in a method of inhibiting MK2 activity in a subject in need thereof, comprising administering to the subject a therapeutically effective amount of the compound, or a pharmaceutically acceptable salt thereof, or a therapeutically effective amount of the pharmaceutical composition.

Clause 156. A compound of any one of clauses 1 to 139, or a pharmaceutically acceptable salt thereof, or a pharmaceutical composition of clause 140 or 141 for use in a method of treating a p38 MAP kinase-mediated disease or disorder in a subject in need thereof, comprising administering to the subject a therapeutically effective amount of the compound, or a pharmaceutically acceptable salt thereof, or a therapeutically effective amount of the pharmaceutical composition.

Clause 157. A compound of any one of clauses 1 to 139, or a pharmaceutically acceptable salt thereof, or a pharmaceutical composition of clause 140 or 141 for use in a method of treating an MK2-mediated disease or disorder in a subject in need thereof, comprising administering to the subject a therapeutically effective amount of the compound, or a pharmaceutically acceptable salt thereof, or a therapeutically effective amount of the pharmaceutical composition.

Clause 158. A compound of any one of clauses 1 to 139, or a pharmaceutically acceptable salt thereof, or a pharmaceutical composition of clause 140 or 141 for use in a method of treating an autoimmune disorder, a chronic inflammatory disorder, an acute inflammatory disorder, an auto-inflammatory disorder, a fibrotic disorder, a metabolic disorder, a neoplastic disorder, a cardiovascular disorder, or a cerebrovascular disorder in a subject in need thereof, comprising administering to the subject a therapeutically effective amount of the compound, or a pharmaceutically acceptable salt thereof, or a therapeutically effective amount of the pharmaceutical composition.

Clause 159. A compound of any one of clauses 1 to 139, or a pharmaceutically acceptable salt thereof, or a pharmaceutical composition of clause 140 or 141 for use in a method of treating an inflammatory condition in a subject in need thereof, comprising administering to the subject a therapeutically effective amount of the compound, or a pharmaceutically acceptable salt thereof, or a therapeutically effective amount of the pharmaceutical composition.

Clause 160. The use of clause 159, wherein the inflammatory condition is selected from inflammatory bowel disease, psoriasis, psoriatic arthritis, rheumatoid arthritis, glomerulonephritis, mixed connective tissue disease (MCTD), dermatomyositis, polymyositis, systemic sclerosis, antineutrophil cytoplasmic antibody-associated vasculitis, anti-phospholipid syndrome, autoimmune hemolytic anemia, macrophage activation syndrome driven inflammatory anemia, IgA nephropathy, type I diabetes, non-alcoholic steatohepatitis, and Sjogren's syndrome.

Clause 161. A compound of any one of clauses 1 to 139, or a pharmaceutically acceptable salt thereof, or a pharmaceutical composition of any one of clauses 86-89 for use in a method of treating rheumatoid arthritis in a subject in need thereof, comprising administering to the subject a therapeutically effective amount of the compound, or a pharmaceutically acceptable salt thereof, or a therapeutically effective amount of the pharmaceutical composition.

Clause 162. The compound for use of any one of clauses 153 to 161, further comprising administering one or more additional therapeutic agents.

Clause 163. The compound for use of clause 162, wherein the one or more additional therapeutic agents is selected from the group consisting of veltuzumab, PF-06835375, eculizumab, milatuzumab, SM-06, SM-03, BT-063, QX-006-N, BOS-161721, AK-101, TNX-1500, theralizumab, daxdilimab, TAK-079, felzartamab, itolizumab, anifrolumab, iscalimab, dapirolizumab pegol, lanalumab, LY-3361237, JNJ-55920839, UBP-1213, DS-7011, PFI-102, BIIB-059, obexelimab, talacotuzumab, vobarilizumab, TE-2324, PRV-3279, chloroquine, hydroxychloroquine, hydroxychloroquine sulfate, COV-08-0064; GNKS-356, AVO-101, rozibafusp alfa, VRN-02, annexuzlimab, ALPN-101, bendamustine hydrochloride, BMS-986256, NKTR-35, atacicept, telitacicept, BMS-986256, M-5049, KZR-616, KPG-818, verdinexor, ALPN-303, valziflocept, LA-1, cenerimod, prednisone, corticotropin, deucravacitinib, CPL-409116, CS-12192, tofacitinib citrate, ISB-830, DV-1079, julemic acid, iberdomide, TAM-01, BML-258, brepocitinib, SDC-1801, SDC-1802, ICP-330, NTR-441, dalazatide, GSK-2646264, SKI-O-703, lanraplenib (GS-9876), GNS-1653, HMPL-523, RSLV-132, interleukin-2 follow-on biologic, interleukin-2 Anteluke, interking recombinant human interleukin-2, ILT-101, CUG-252, DZ-2002, PEGylated HLA-x (SLE), AC-0058, fenebrutinib, XNW-1011, tirabrutinib hydrochloride, branebrutinib, elsubrutinib, orelabrutinib, DWP-213388, INV-103, R-salbutamol sulphate, anchorins, NIK-SMI1, X-6, INV-17, Oshadi D, baricitinib, upadacitinib, filgotinib, itacitinib, INCB-54707, delgocitinib, DWP-212525, CKD-971, as mometasone, betamethasone, forigerimod, anandamide, DCB-SLE1, arsenic trioxide, tairuimide, TV-4710 (edratide), allogeneic human umbilical cord-derived mesenchymal stem cell therapy (hUC-MSCs), LC-200, BI-705564, SM-934, GX-101, TXR-712, TXR-711, CIT-013, MHV-370, Panzyga®, TPX-6001, TPX-7001, artenimol, and AMG-592, or a pharmaceutically acceptable salt thereof.

Clause 164. The compound for use of any one of clauses 153 to 163, wherein the subject is a human.

### VI. Compound Preparation

Some embodiments of the present disclosure are directed to processes and intermediates useful for preparing the compounds provided herein or pharmaceutically acceptable salts thereof.

Compounds described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography.

During any of the processes for preparation of the compounds provided herein, it may be necessary and/or desirable to protect sensitive or reactive groups on any of the molecules concerned. This may be achieved by means of conventional protecting groups as described in standard works, such as T. W. Greene and P. G. M. Wuts, “Protective Groups in Organic Synthesis,” 4^{th} ed., Wiley, New York 2006. The protecting groups may be removed at a convenient subsequent stage using methods known from the art.

Exemplary chemical entities useful in methods of the embodiments will now be described by reference to illustrative synthetic schemes for their general preparation herein and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups. Each of the reactions depicted in the general schemes is preferably run at a temperature from about 0° C. to the reflux temperature of the organic solvent used.

The methods of the present disclosure generally provide a specific enantiomer or diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or diastereomerically pure, or can be a mixture of enantiomers or diastereomers.

Representative syntheses of compounds of the present disclosure are described in the schemes below, and the particular examples that follow.

### VIII. Examples

Certain abbreviations and acronyms are used in describing the experimental details. Although a person of ordinary skill in the art will readily recognize and understand most of the abbreviations and acronyms, the below list provides many of the meanings of the abbreviations and acronyms.

### List of Abbreviations and Acronyms

| Abbreviation | Meaning |
|---|---|
| ° C. | Degree Celsius |
| Ac | acetyl |
| ACN, MeCN | acetonitrile |
| AcOH | Acetic acid |
| ATP | Adenosine-5′-triphosphate |
| B₂pin₂ | bis(pinacolato)diboron |
| Bn | Benzyl |
| Boc | Tert-butyloxycarbonyl |
| Boc₂O | di-tert-butyl dicarbonate |
| Bpin | (pinacolato)boron |
| br | broad |
| Bu | butyl |
| BrettPhos | 2-(Dicyclohexylphosphino)3,6-dimethoxy-2′,4′,6′-triisopropyl-1,1′-biphenyl |
| BrettPhos Pd G3 | [2′-(Amino-κN)[1,1′-biphenyl]-2-yl-κC][dicyclohexyl[3,6-dimethoxy-2′,4′,6′- |
| | tris(1-methylethyl)[1,1′-biphenyl]-2-yl]phosphine-κP](methanesulfonato- |
| | κO)palladium |
| Bz | benzoyl |
| BzCl | benzoyl chloride |
| CataCXium | Di(1-adamantyl)-n-butylphosphine |
| CataCXium Pd G3 | [(Di(1-adamantyl)-butylphosphine)-2-(2′-amino-1,1′-biphenyl)]palladium(II) |
| | methanesulfonate |
| CataCXium Pd G4 | [di(adamantan-1-yl)(butyl)phosphine](methanesulfonato-κO)[2′- |
| | (methylamino)-2-biphenylyl]palladium |
| Cbz | Carboxybenzyl |
| CDI | 1,1′-Carbonyldiimidazole |
| Cy | cyclohexyl |
| CyBu | cyclobutyl |
| CyPr | cyclopropyl |
| d | Doublet |
| DAST | Bis(2-methoxyethyl)aminosulfur trifluoride |
| dba | dibenzalacetone |
| DBU | 1,8-Diazabicyclo[5.4.0]undec-7-ene |
| DCE, 1,2-DCE | 1,2-dichloroethane |
| DCM | dichloromethane |
| dd | Doublet of doublets |
| ddd | Doublet of doublet of doublets |
| DEAD | Diethyl azodicarboxylate |
| DIAD | Diisopropyl azodicarboxylate |
| DIC | N,N′-Diisopropylcarbodiimide |
| DIPEA, DIEA | N,N-diisopropylethylamine |
| DMAC | N,N-dimethylacetamide |
| DMAP | 4-dimethylaminopyridine |
| DME, 1,2-DME | dimethoxyethane |
| DMEM | Dulbecco′s Modified Eagle Medium |
| DMF | N,N-dimethylformamide |
| DMPU | 1,3-dimethyl-3,4,5,6-tetrahydro-2(1H)-pyrimidinone |
| DMSO | dimethylsulfoxide |
| DPPA | Diphenylphosphoryl azide |
| dppf | 1,1′-Ferrocenediyl-bis(diphenylphosphine) |
| EDC | 1-Ethyl-3-(3-dimethylaminopropyl)carbodiimide |
| EDTA | Ethylenediaminetetraacetic acid |
| equiv/eq | Equivalents |
| ES/MS | electron spray mass spectrometry |
| ESI | Electrospray ionization |
| Et | ethyl |
| EtOAc | ethylacetate |
| EtOH | ethanol |
| FBS | fetal bovine serum |
| g | Grams |
| h, hr, or hrs | Hour or hours |
| HATU | 2-(7-Aza-1H-Benzotriazole-1-yl)-1,1,3,3-tetramethyluronium |
| | hexafluorophosphate |
| HCl | Hydrochloric acid |
| HPLC | high performance liquid chromatography |
| Hunig′s Base | N,N-diisopropylethylamine |
| Hz | Hertz |
| IC₅₀ | The half maximal inhibitory concentration |
| IPA, iPrOH | isopropanol |
| iPr | isopropyl |
| J | Coupling constant |
| kg | Kilogram |
| KOtBu | potassium tert-butoxide |
| LC | liquid chromatography |
| LC/MS, LCMS | Liquid chromatography mass spectrometry |
| LDA | Lithium diisopropylamide |
| LiHMDS, LHMDS | Lithium bis(trimethylsilyl)amide |
| M | Molar |
| m | Multiplet |
| m/z | mass-to-charge ratio |
| M+ | Mass peak |
| M + H | Mass peak plus hydrogen |
| mCPBA | meta-chloroperbenzoic acid |
| Me | methyl |
| MeOH | methanol |
| Mg | Milligram |
| MHz | Megahertz |
| min, m | Minute |
| mL | Milliliter |
| mM | Millimolar |
| mmol | Millimole |
| Mol | Mole |
| MS | Mass spectroscopy |
| Ms | methanesulfonyl |
| MTBE | Methyl tert-butyl ether |
| Mw | Microwave |
| N | Normal |
| NaHMDS, NHMDS | Sodium bis(trimethylsilyl)amide |
| NaOtBu | sodium tert-butoxide |
| NBS | N-bromosuccinimide |
| NCS | N-chlorosuccinimide |
| NIS | N-iodosuccinimide |
| NMP | N-methyl-2-pyrrolidone |
| NMR | Nuclear magnetic resonance |
| OAc | acetate |
| p | Pentuplet |
| PCR | Polymerase chain reaction |
| PCy₃ | tricyclohexylphosphine |
| Pd SPhos G3 | [2′-(Amino-κN)[1,1′-biphenyl]-2-yl-κC][dicyclohexyl(2′,6′-dimethoxy[1,1′- |
| | biphenyl]-2-yl)phosphine-κP](methanesulfonato-κO)palladium |
| Ph | phenyl |
| Ph₃P, PPh₃ | triphenylphosphine |
| PhMe, Tol | toluene |
| pin | pinacol |
| ppm | Parts per million |
| PPTS | Pyridinium para-toluenesulfonate |
| Prep | Preparative |
| PtBu₃ | tributylphosphine |
| Pyr | pyridine |
| RBF | round bottom flask |
| Rf | Retention factor |
| RP | Reverse phase |
| (R)-Pheox Ru(II) | Tetrakis(acetonitrile)[2-[(4R)-4,5-dihydro-4-phenyl-2-oxazolyl- |
| | N]phenyl]ruthenium(II) Hexafluorophosphate |
| RT | room temperature |
| RuPhos | 2-Dicyclohexylphosphino-2′,6′-diisopropoxy biphenyl |
| RuPhos Pd G3 | [2′-(Amino-κN)[1,1′-biphenyl]-2-yl-κC][2′,6′-bis(1-methylethoxy)[1,1′- |
| | biphenyl]-2-yl]dicyclohexylphosphine-κP](methanesulfonato-κO)palladium |
| s | Second |
| s | Singlet |
| SEM | Trimethylsilylethoxymethyl |
| SFC | supercritical fluid chromatography |
| Sn₂Bu₆ | hexabutylditin |
| Soln | solution |
| SPhos | 2-Dicyclohexylphosphino-2′,6′-dimethoxy biphenyl |
| SPhos Pd G4 | (SP-4-3)-[Dicyclohexyl(2′,6′-dimethoxy[1,1′-biphenyl]-2-yl)phosphine- |
| | κP](methanesulfonato-κO)[2′-(methylamino-κN)[1,1′-biphenyl]-2-yl- |
| | κC]palladium |
| t | Triplet |
| TBAF | Tetrabutylammonium fluoride |
| TBAI | Tetrabutylammonium iodide |
| TBDPS | Tert-butyldiphenylsilyl |
| TBS | Tert-butyldimethylsilyl |
| tBu | tert-butyl |
| tBuOH | tert-butanol |
| TEA, Et₃N | triethylamine |
| Tf | Trifluoromethylsulfonyl |
| TFA | 1,1, 1-trifluoroacetic acid |
| THF | tetrahydrofuran |
| THP | Tetrahydropyranyl |
| TLC | Thin layer chromatography |
| TMS | Trimethylsilyl |
| Ts | 4-toluenesulfonyl |
| XantPhos | 4,5-Bis(diphenylphosphino)-9,9-dimethylxanthene |
| XPhos | 2-Dicyclohexylphosphino-2′,4′,6′-triisopropylbiphenyl |
| XPhos Pd G2 | (SP-4-4)-[2′-(Amino-κN)[1,1′-biphenyl]-2-yl-κC]chloro[dicyclohexyl[2′,4′,6′- |
| | tris(1-methylethyl)[1,1′-biphenyl]-2-yl]phosphine]palladium |
| XPhos Pd G4 | (SP-4-3)-[Dicyclohexy][2′,4′,6′-tris(1-methylethyl)[1,1′-biphenyl]-2- |
| | yl]phosphine](methanesulfonato-κO)[2′-(methylamino-κN)[1,1′-biphenyl]-2- |
| | yl-κC]palladium |
| δ | parts per million referenced to residual solvent peak |
| μg | Microgram |
| μL/μl | Microliter |
| μM | Micromolar |
| μm | Micrometer |
| μmol | Micromole |

Exemplary chemical entities of the present disclosure are provided in the specific examples that follow. Those skilled in the art will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

The Examples provided herein describe the synthesis of compounds disclosed herein as well as intermediates used to prepare the compounds. It is to be understood that individual steps described herein may be combined. It is also to be understood that separate batches of a compound may be combined and then carried forth in the next synthetic step.

In the following description of the Examples, specific embodiments are described. These embodiments are described in sufficient detail to enable those skilled in the art to practice certain embodiments of the present disclosure. Other embodiments may be utilized and logical and other changes may be made without departing from the scope of the disclosure. The following description is, therefore, not intended to limit the scope of the present disclosure.

### Intermediates

The following intermediates were purchased from commercial vendors:

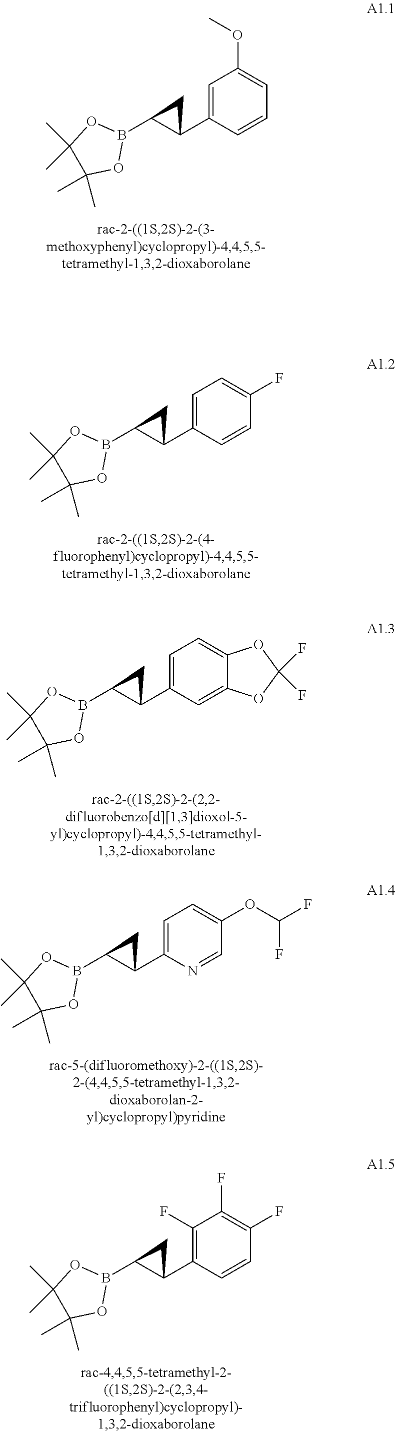 [SMILES: COc1cccc([C@H]2C[C@@H]2B2OC(C)(C)C(C)(C)O2)c1 ; CC1(C)OB([C@H]2C[C@@H]2c2ccc(F)cc2)OC1(C)C ; CC1(C)OB([C@H]2C[C@@H]2c2ccc3c(c2)OC(F)(F)O3)OC1(C)C ; CC1(C)OB([C@H]2C[C@@H]2c2ccc(OC(F)F)cn2)OC1(C)C ; CC1(C)OB([C@H]2C[C@@H]2c2ccc(F)c(F)c2F)OC1(C)C]

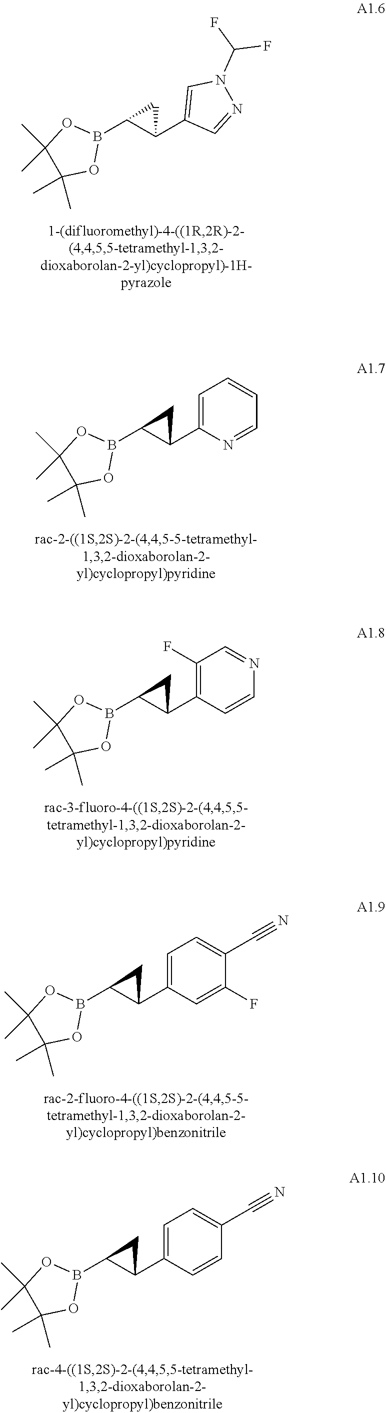 [SMILES: CC1(C)OB([C@@H]2C[C@H]2c2cnn(C(F)F)c2)OC1(C)C ; CC1(C)OB([C@H]2C[C@@H]2c2ccccn2)OC1(C)C ; CC1(C)OB([C@H]2C[C@@H]2c2ccncc2F)OC1(C)C ; CC1(C)OB([C@H]2C[C@@H]2c2ccc(C#N)c(F)c2)OC1(C)C ; CC1(C)OB([C@H]2C[C@@H]2c2ccc(C#N)cc2)OC1(C)C]

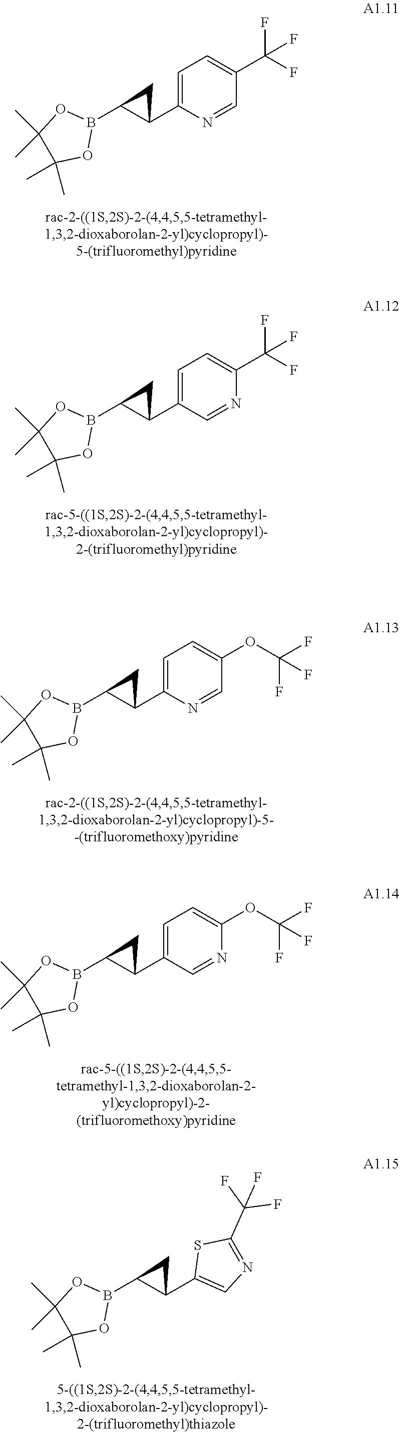 [SMILES: CC1(C)OB([C@H]2C[C@@H]2c2ccc(C(F)(F)F)cn2)OC1(C)C ; CC1(C)OB([C@H]2C[C@@H]2c2ccc(C(F)(F)F)nc2)OC1(C)C ; CC1(C)OB([C@H]2C[C@@H]2c2ccc(OC(F)(F)F)cn2)OC1(C)C ; CC1(C)OB([C@H]2C[C@@H]2c2ccc(OC(F)(F)F)nc2)OC1(C)C ; CC1(C)OB([C@H]2C[C@@H]2c2cnc(C(F)(F)F)s2)OC1(C)C]

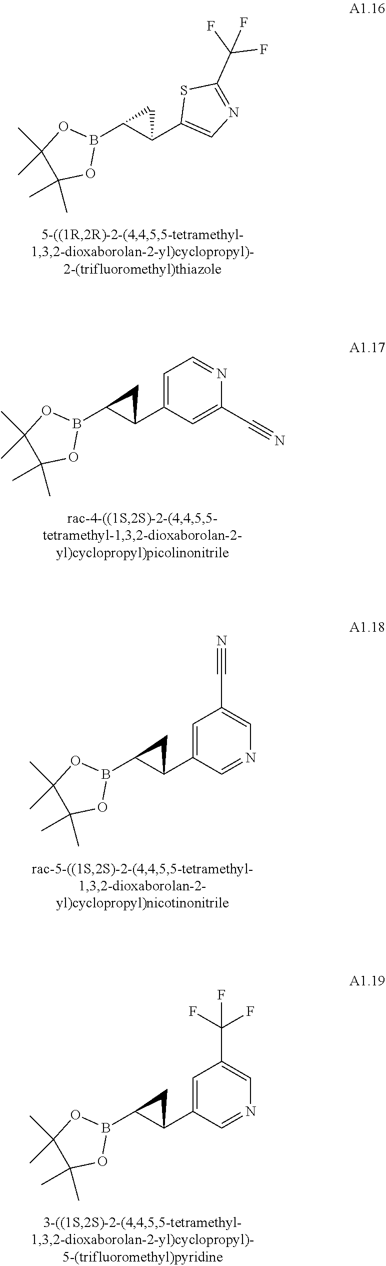 [SMILES: CC1(C)OB([C@@H]2C[C@H]2c2cnc(C(F)(F)F)s2)OC1(C)C ; CC1(C)OB([C@H]2C[C@@H]2c2ccnc(C#N)c2)OC1(C)C ; CC1(C)OB([C@H]2C[C@@H]2c2cncc(C#N)c2)OC1(C)C ; CC1(C)OB([C@H]2C[C@@H]2c2cncc(C(F)(F)F)c2)OC1(C)C]

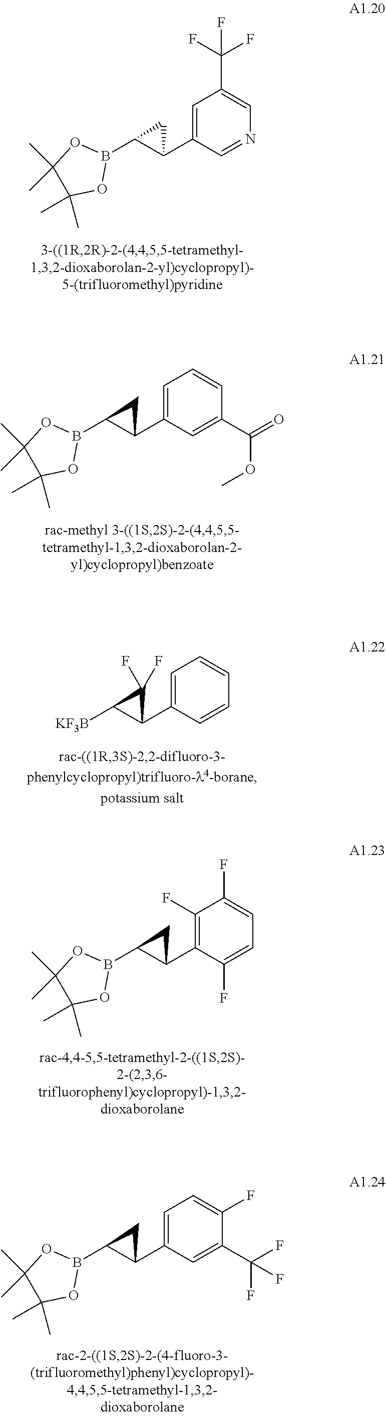

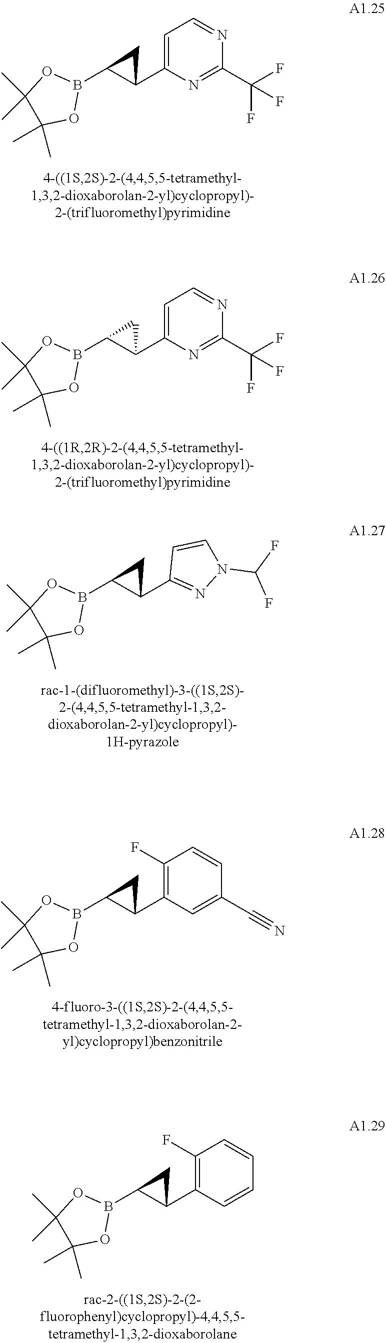 [SMILES: CC1(C)OB([C@H]2C[C@@H]2c2ccnc(C(F)(F)F)n2)OC1(C)C ; CC1(C)OB([C@@H]2C[C@H]2c2ccnc(C(F)(F)F)n2)OC1(C)C ; CC1(C)OB([C@H]2C[C@@H]2c2ccn(C(F)F)n2)OC1(C)C ; CC1(C)OB([C@H]2C[C@@H]2c2cc(C#N)ccc2F)OC1(C)C ; CC1(C)OB([C@H]2C[C@@H]2c2ccccc2F)OC1(C)C]

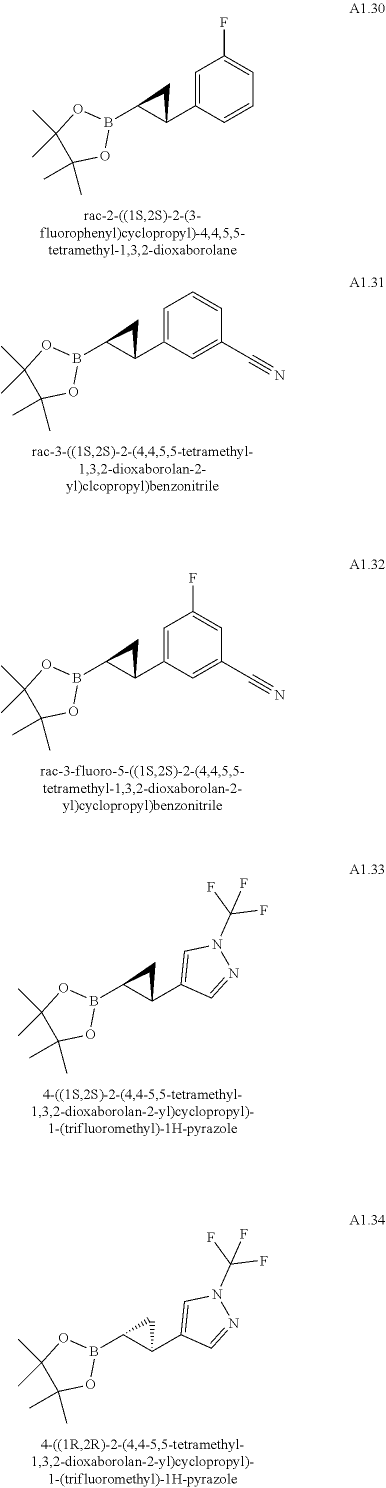 [SMILES: CC1(C)OB([C@H]2C[C@@H]2c2cccc(F)c2)OC1(C)C ; CC1(C)OB([C@H]2C[C@@H]2c2cccc(C#N)c2)OC1(C)C ; CC1(C)OB([C@H]2C[C@@H]2c2cc(F)cc(C#N)c2)OC1(C)C ; CC1(C)OB([C@H]2C[C@@H]2c2cnn(C(F)(F)F)c2)OC1(C)C ; CC1(C)OB([C@@H]2C[C@H]2c2cnn(C(F)(F)F)c2)OC1(C)C]

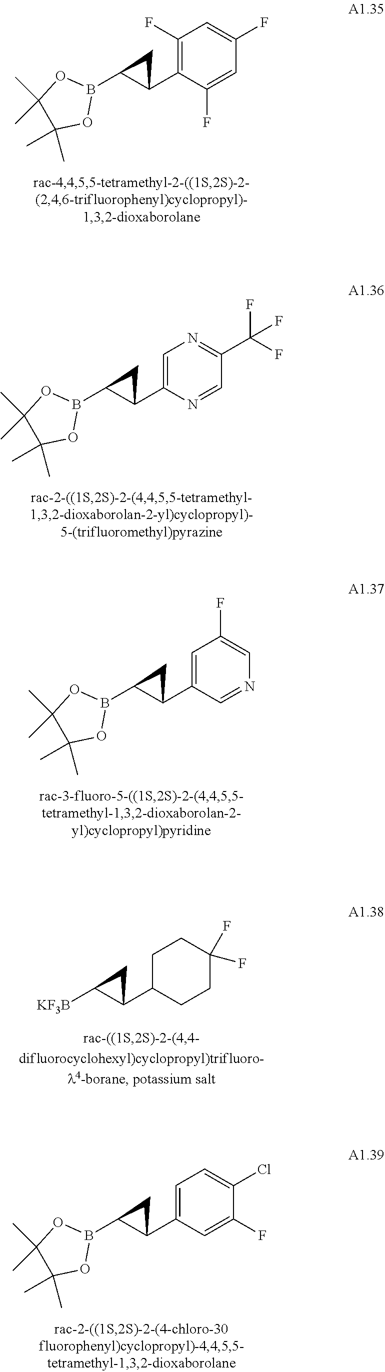

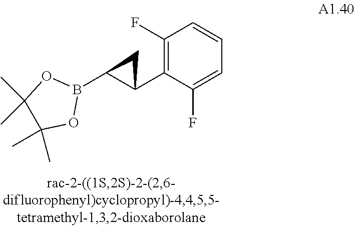 [SMILES: CC1(C)OB([C@H]2C[C@@H]2c2c(F)cccc2F)OC1(C)C]

### Preparation of rac-4,4,5,5-tetramethyl-2-((1S,2S)-2-(3,4,5-trifluorophenyl)cyclopropyl)-1,3,2-dioxaborolane (A2.1)

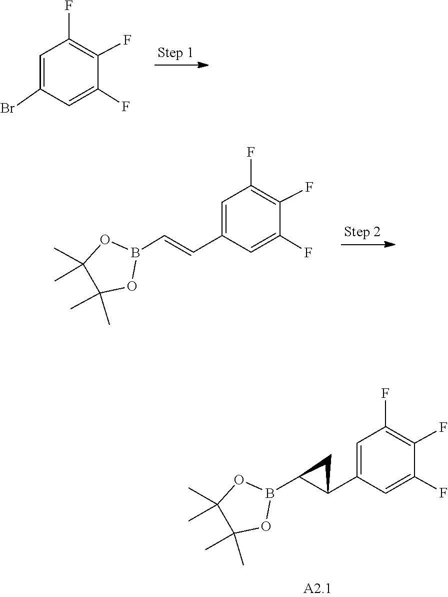 [SMILES: Fc1cc(Br)cc(F)c1F ; CC1(C)OB(/C=C/c2cc(F)c(F)c(F)c2)OC1(C)C ; CC1(C)OB([C@H]2C[C@@H]2c2cc(F)c(F)c(F)c2)OC1(C)C]

Step 1. To 5-bromo-1,2,3-trifluorobenzene (15.0 g, 71.1 mmol) in toluene (42 mL) was added Pd₂(dba)₃ (6.51 g, 7.11 mmol), DIPEA (24.7 mL, 142 mmol), P(t-Bu)₃ (4.13 g, 14.2 mmol), and vinylboronic acid pinacol ester (12.0 g, 78.2 mmol). The mixture was degassed with N₂ and was then stirred at 95° C. for 3 h. The mixture was concentrated. The residue was purified via flash column chromatography on silica gel to afford (E)-4,4,5,5-tetramethyl-2-(3,4,5-trifluorostyryl)-1,3,2-dioxaborolane.

Step 2. To a flask containing DCM (60 mL) under N₂ was added Et₂Zn (42.2 mL of a 1.00 M soln in toluene, 42.2 mmol). The mixture was cooled to 0° C. TFA (2.09 mL, 28.1 mmol) was added, dropwise, over 30 min to maintain an internal temperature <10° C. The resulting mixture was stirred at 0° C. for 30 min. Diiodomethane (4.54 mL, 56.3 mmol) was added, dropwise, over 30 min to the mixture at 3° C. The mixture was stirred at rt for 30 min. The mixture was cooled to 3° C. and a solution of (E)-4,4,5,5-tetramethyl-2-(3,4,5-trifluorostyryl)-1,3,2-dioxaborolane (8.00 g, 28.1 mmol) in DCM (40 mL) was added in one portion. The mixture was stirred at rt for 10 h. The reaction was quenched by the addition of HCl (50.0 mL of a 0.5 N solution). The mixture was extracted with DCM. The combined organic layers were washed with brine and then concentrated. The residue was purified by flash column chromatography on silica gel to yield rac-4,4,5,5-tetramethyl-2-((1S,2S)-2-(3,4,5-trifluorophenyl)cyclopropyl)-1,3,2-dioxaborolane (A2.1). ¹H NMR (CDCl₃, 400 MHz): δ 6.69-6.65 (m, 2H), 2.05-1.57 (m, 1H), 1.26 (s, 12H), 1.22-1.17 (m, 1H), 0.20-0.23 (m, 1H).

rac-4,4,5,5-tetramethyl-2-((1S,2S)-2-(2,4,5-trifluorophenyl)cyclopropyl)-1,3,2-dioxaborolane (A2.2). A2.2 was prepared analogously to A2.1, using 1-bromo-2,4,5-trifluorobenzene. ¹H NMR (CDCl₃, 400 MHz): δ 6.88-6.81 (m, 1H), 6.73-6.68 (m, 1H), 2.24-2.19 (m, 1H), 1.25 (s, 12H), 1.18-1.15 (m, 1H), 0.96-0.95 (m, 1H), 0.23-0.21 (m, 1H).

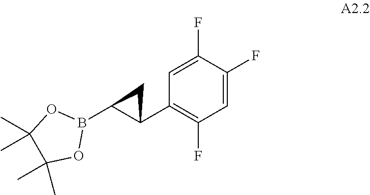 [SMILES: CC1(C)OB([C@H]2C[C@@H]2c2cc(F)c(F)cc2F)OC1(C)C]

### Preparation of rac-2-((1S,2R)-2-benzylcyclopropyl)-4,4,5,5-tetramethyl-1,3,2-dioxaborolane (A3.1)

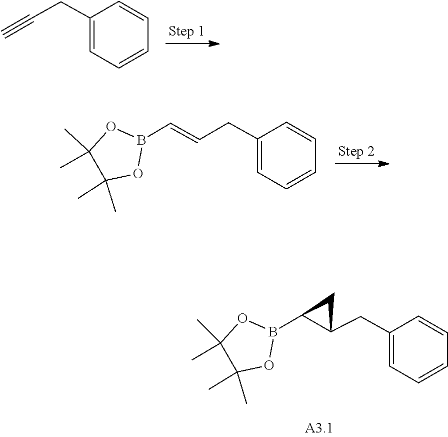

Step 1. To a solution of prop-2-yn-1-ylbenzene (16.0 mL, 129 mmol) in toluene (100 mL) was added AgOAc (2.16 g, 12.9 mmol) and pinacolborane (28.1 mL, 193 mmol). The mixture was stirred at 120° C. for 16 h. The mixture was concentrated. The residue was partitioned between water and DCM and the aqueous layer was extracted with DCM. The combined organic layers were washed with brine and then concentrated. The residue was purified via flash column chromatography on silica gel to yield (E)-4,4,5,5-tetramethyl-2-(3-phenylprop-1-en-1-yl)-1,3,2-dioxaborolane. ¹H NMR (CDCl₃, 400 MHz): δ 1.22 (s, 11H) 3.45 (dd, J=6.24, 1.34 Hz, 2H) 5.42 (dt, J=17.85, 1.59 Hz, 1H) 6.73 (dt, J=17.85, 6.36 Hz, 1H) 7.11-7.19 (m, 3H) 7.20-7.26 (m, 2H).

Step 2. rac-2-((1S,2R)-2-benzylcyclopropyl)-4,4,5,5-tetramethyl-1,3,2-dioxaborolane (A3.1) was prepared via the method described in Step 2 in the synthesis of A2.1, using (E)-4,4,5,5-tetramethyl-2-(3-phenylprop-1-en-1-yl)-1,3,2-dioxaborolane. ¹H NMR (CDCl₃, 400 MHz): δ 0.46-0.62 (m, 1H) 0.70 (br d, J=5.65 Hz, 1H) 0.86-0.99 (m, 1H) 1.11-1.23 (m, 12H) 1.45-1.89 (m, 1H) 2.44-2.61 (m, 1H) 3.45-3.67 (m, 1H) 7.05-7.31 (m, 6H).

### Preparation of 1,3-dioxoisoindolin-2-yl 2-diazoacetate (A4.1)

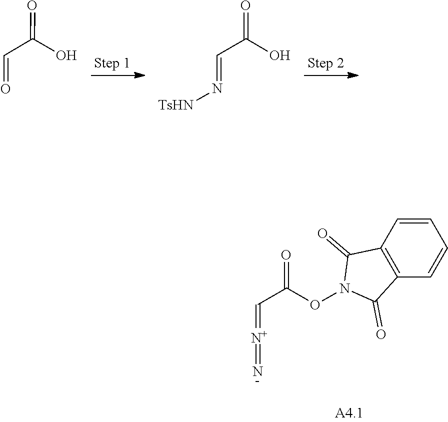

Step 1. To a solution of p-toluenesulfonylhyrazide (95.4 g, 644 mmol) in water (500 mL) was added glyoxylic acid (100 g, 536 mmol). The mixture was stirred at 60° C. for 2 h. The mixture was cooled to 0° C. and filtered. The filter cake was dried under vacuum to afford (E)-2-(2-tosylhydrazineylidene)acetic acid. ¹H NMR (DMSO-d₆, 400 MHz) δ12.28 (br s, 1H), 7.71 (d, J=8.0 Hz, 2H), 7.43 (d, J=8.0 Hz, 2H), 7.19 (s, 1H), 2.38 (s, 3H).

Step 2. To a solution of (E)-2-(2-tosylhydrazineylidene)acetic acid (100 g, 413 mmol) in DCM (500 mL) at 0° C. was added DMF (1.27 mL, 16.5 mmol) followed by oxalyl chloride (54.2 mL, 619 mmol). The mixture was stirred at rt for 16 h. The mixture was concentrated. The residue was dissolved in DCM (1.00 L) and cooled to 0° C. N-hydroxyphthalimide (101 g, 619 mmol) was added followed by 2,6-lutidine (96.1 mL, 825 mmol). The mixture was stirred at rt for 6 h. The mixture was concentrated and the residue was purified by flash column chromatography on silicagel to yield 1,3-dioxoisoindolin-2-yl 2-diazoacetate (A4.1). ¹H NMR (CDCl₃, 400 MHz): δ 7.91 (dd, J=5.2, 3.2 Hz, 2H), 7.78-7.83 (m, 2H), 5.20 (br s, 1H).

### Preparation of 2-methyl-4-((1S,2S)-2-(4,4,5,5-tetramethyl-1,3,2-dioxaborolan-2-yl)cyclopropyl)thiazole (A5.1)

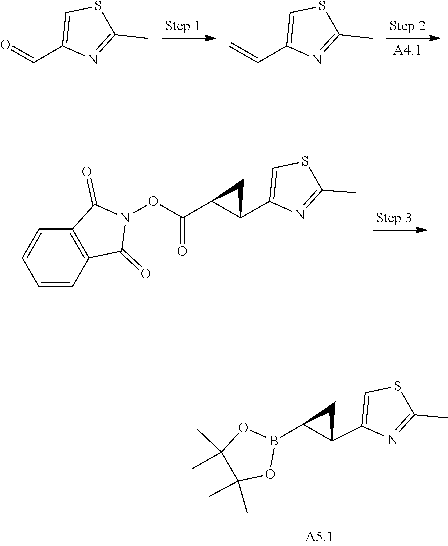

Step 1. To methyltriphenylphosphonium bromide (2.00 g, 5.60 mmol) in THF (8.0 mL) at 0° C. was added NaHMDS (5.6 mL of a 1 M soln in THF, 5.6 mmol). The mixture was stirred at rt for 1 h. The mixture was cooled to 0° C. and a solution of 2-methylthiazole-4-carbaldehyde (0.445 g, 3.50 mmol) in THF (4 mL) was added, dropwise. The mixture was stirred at 0° C. for 2 h. The reaction was quenched by the addition of sat NH₄Cl soln. The mixture was diluted with EtOAc and the aqueous layer was extracted with EtOAc. The combined organic layers were washed with brine, dried (Na₂SO₄), filtered, and concentrated. The residue was purified by flash column chromatography on silica gel to afford 2-methyl-4-vinylthiazole. ¹H NMR (400 MHz, Chloroform-d) δ 6.94 (s, 1H), 6.68 (dd, J=17.3, 10.9 Hz, 1H), 6.01 (dd, J=17.3, 1.6 Hz, 1H), 5.34 (dd, J=10.9, 1.6 Hz, 1H), 2.72 (s, 3H).

Step 2. A solution of 2-methyl-4-vinylthiazole (0.141 g, 1.13 mmol) and (R)-Pheox Ru(II) (0.036 g, 0.056 mmol) in DCM (11 mL) was sparged with N₂ for approximately 1 min and then cooled to 0° C. A solution of 1,3-dioxoisoindolin-2-yl 2-diazoacetate (0.287 g, 1.24 mmol) in DCM (6.2 mL) was added, dropwise, over 15 min. The mixture was stirred at 0° C. for 1 h and then at rt for 16 h. The reaction was quenched by the addition of MeOH and the mixture was concentrated. The residue was purified via flash column chromatography on silica gel to give 1,3-dioxoisoindolin-2-yl (1S,2S)-2-(2-methylthiazol-4-yl)cyclopropane-1-carboxylate. ES/MS: m/z 329.2 [M+H]⁺.

Step 3. A solution of 1,3-dioxoisoindolin-2-yl (1S,2S)-2-(2-methylthiazol-4-yl)cyclopropane-1-carboxylate (0.183 g, 0.557 mmol) in α,α,α-trifluorotoluene (2.8 mL) was sparged with N₂ for 5 minutes. B₂pin₂ (0.283 g, 1.11 mmol) was added, followed by tert-butyl isonicotinate (0.05 mL, 0.279 mmol). The mixture was stirred at 30° C. for 16 h. The mixture was sparged with air for 5 minutes and then filtered through Celite. The filtrate was concentrated. The residue was purified via flash column chromatography on silica gel to afford 2-methyl-4-((1S,2S)-2-(4,4,5,5-tetramethyl-1,3,2-dioxaborolan-2-yl)cyclopropyl)thiazole (A5.1). ES/MS: m/z 266.2 [M+H]⁺.

2-cyclopropyl-6-((1S,2S)-2-(4,4,5,5-tetramethyl-1,3,2-dioxaborolan-2-yl)cyclopropyl)pyridine (A5.2). A5.2 was prepared analogously to A5.1, using 6-cyclopropylpicolinaldehyde. ¹H NMR (400 MHz, Chloroform-d) δ 7.33 (t, J=7.6 Hz, 1H), 6.87 (t, J=7.6 Hz, 2H), 2.13 (dt, J=7.9, 5.1 Hz, 1H), 1.89 (td, J=8.2, 4.2 Hz, 1H), 1.30-1.19 (m, 13H), 1.06 (td, J=7.3, 2.6 Hz, 1H), 1.01-0.90 (m, 2H), 0.84 (dd, J=8.3, 2.7 Hz, 2H), 0.64 (ddd, J=10.0, 6.9, 5.2 Hz, 1H).

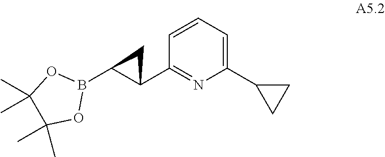 [SMILES: CC1(C)OB([C@H]2C[C@@H]2c2cccc(C3CC3)n2)OC1(C)C]

4,4,5,5-tetramethyl-2-((1S,2S)-2-phenylcyclopropyl)-1,3,2-dioxaborolane (A5.3). A5.3 was prepared from styrene using procedures described in Steps 2 and 3 in the synthesis of A5.1. ¹H NMR (400 MHz, Chloroform-d) δ 7.25-7.21 (m, 2H), 7.16-7.10 (m, 1H), 7.10-7.04 (m, 2H), 2.10 (dt, J=8.1, 5.4 Hz, 1H), 1.25 (s, 6H), 1.24 (s, 6H), 1.16 (ddd, J=8.1, 6.8, 3.7 Hz, 1H), 1.00 (ddd, J=9.8, 5.3, 3.7 Hz, 1H), 0.30 (ddd, J=9.8, 6.8, 5.5 Hz, 1H).

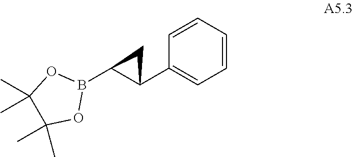 [SMILES: CC1(C)OB([C@H]2C[C@@H]2c2ccccc2)OC1(C)C]

### Preparation of 2-((1S,2S)-2-(3,4-difluorophenyl)cyclopropyl)-4,4,5,5-tetramethyl-1,3,2-dioxaborolane (A6.1)

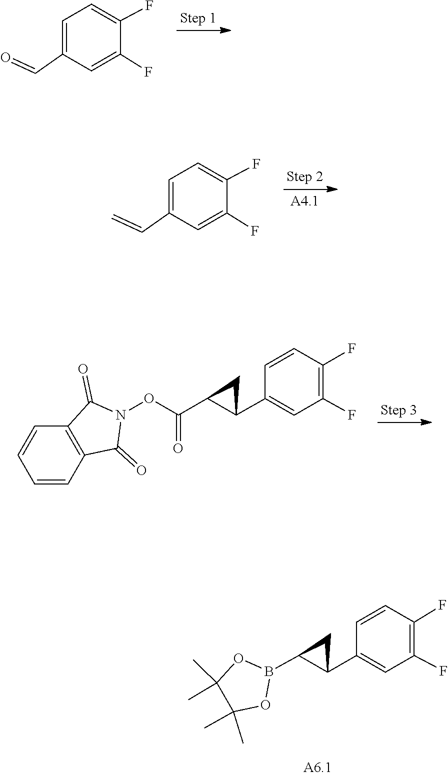

Step 1. 1,2-difluoro-4-vinylbenzene was prepared from 3,4-difluorobenzaldehyde following the procedure described in Step 1 in the synthesis of A5.1.

Step 2. 1,3-dioxoisoindolin-2-yl (1S,2S)-2-(3,4-difluorophenyl)cyclopropane-1-carboxylate was synthesized from 1,2-difluoro-4-vinylbenzene following the procedure described in Step 2 of A5.1.

Step 3. To 1,3-dioxoisoindolin-2-yl (1S,2S)-2-(3,4-difluorophenyl)cyclopropane-1-carboxylate (0.500 g, 1.46 mmol) in DMF (5.00 mL) was added B₂cat₂ (0.519 g, 2.18 mmol). The reaction vessel was flushed with Ar and sealed. The reaction mixture was stirred under blue LED lights for 6 h and was then cooled to rt. Pinacol (0.688 g, 5.83 mmol) was added and the mixture was stirred at rt for 1.5 h. Saturated NH₄Cl soln was added to the reaction mixture and the aqueous layer was extracted with EtOAc. The organic layer was dried (Na₂SO₄), filtered, and concentrated. The residue was purified via flash column chromatography to afford 2-((1S,2S)-2-(3,4-difluorophenyl)cyclopropyl)-4,4,5,5-tetramethyl-1,3,2-dioxaborolane (A6.1). ¹H NMR (400 MHz, Chloroform-d) δ 7.01 (dt, J=10.4, 8.4 Hz, 1H), 6.75-6.89 (m, 2H), 2.06 (dt, J=8.0, 5.2 Hz, 1H), 1.25 (d, J 3.6 Hz, 12H), 1.16 (ddd, J=8.0, 6.8, 3.6 Hz, 1H), 0.95 (ddd, J=9.6, 5.2, 3.6 Hz, 1H), 0.20-0.29 (m, 1H).

2-((1S,2S)-2-(2,4-difluorophenyl)cyclopropyl)-4,4,5,5-tetramethyl-1,3,2-dioxaborolane (A6.2). A6.2 was prepared analogously to A6.1, using 2,4-difluorobenzaldehyde. ¹H NMR (400 MHz, Chloroform-d) δ 6.82-6.94 (m, 1H), 6.68-6.80 (m, 2H), 2.19-2.28 (m, 1H), 1.26 (s, 12H), 1.12-1.19 (m, 1H), 0.99 (ddd, J=9.6, 5.6, 3.6 Hz, 1H), 0.24 (dt, J=9.6, 6.4 Hz, 1H).

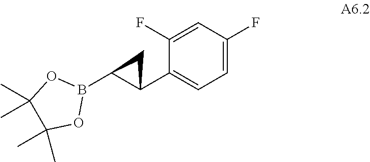 [SMILES: CC1(C)OB([C@H]2C[C@@H]2c2ccc(F)cc2F)OC1(C)C]

### Preparation of 2-((1S,2S)-2-(3-chloro-4-fluoro phenyl)cyclopropyl)-4,4,5,5-tetramethyl-1,3,2-dioxaborolane (A7.1)

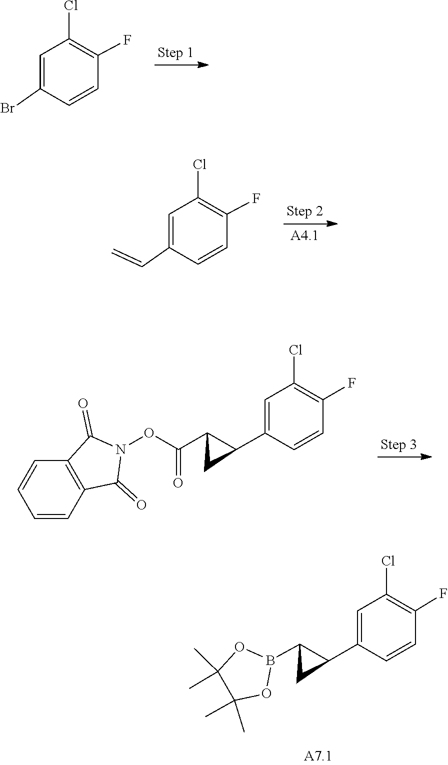

Step 1. To a solution of 4-bromo-2-chloro-1-fluorobenzene (5.0 g, 23.8 mmol, 2.9 mL, 1 eq) in dioxane (50 mL) and H₂O (10 mL) were added potassium trifluoro(vinyl)borate (6.4 g, 47.7 mmol, 2 eq), Pd(dppf)Cl₂ (3.4 g, 4.7 mmol, 0.2 eq), and Cs₂CO₃ (15.5 g, 47.7 mmol, 2 eq). The reaction mixture was degassed and purged with N₂ for three times. The mixture was stirred at 100° C. for 4 h under N₂ atmosphere. The reaction mixture was cooled to room temperature diluted with water (30 mL), then the mixture was extracted with ethyl acetate (60 mL×2). The combined organics were washed with brine (60 mL), dried over Na₂SO₄, concentrated under reduced pressure to give a residue. The residue was purified by flash column (SiO₂, 0-5% ethyl acetate in petroleum ether) to afford 2-chloro-1-fluoro-4-vinylbenzene. 1HNMR (400 MHz, CHLOROFORM-d) δ=7.45 (dd, J=2.1, 7.0 Hz, 1H), 7.27 (s, 1H), 7.19-7.05 (m, 1H), 6.63 (dd, J=10.9, 17.5 Hz, 1H), 5.69 (d, J=17.5 Hz, 1H), 5.33-5.24 (m, 1H)

Step 2. 1,3-dioxoisoindolin-2-yl (1S,2S)-2-(3-chloro-4-fluorophenyl)cyclopropane-1-carboxylate was prepared analogously to the procedure described in Step 2 of A5.1, using 2-chloro-1-fluoro-4-vinylbenzene.

Step 3. 2-((1S,2S)-2-(3-chloro-4-fluorophenyl)cyclopropyl)-4,4,5,5-tetramethyl-1,3,2-dioxaborolane (A7.1) was prepared analogously to the procedure described in Step 3 of A5.1, using 1,3-dioxoisoindolin-2-yl (1S,2S)-2-(3-chloro-4-fluorophenyl)cyclopropane-1-carboxylate. 2-chloro-6-((1S,2S)-2-(4,4,5,5-tetramethyl-1,3,2-dioxaborolan-2-yl)cyclopropyl)pyridine (A7.2). A7.2 was prepared analogously to 7.1, using 2-bromo-6-chloropyridine. 1H NMR (400 MHz, DMSO-d6) δ=7.69 (t, J=7.8 Hz, 1H), 7.35 (d, J=7.6 Hz, 1H), 7.24 (d, J=7.9 Hz, 1H), 2.18 (td, J=5.1, 7.8 Hz, 1H), 1.18 (d, J=5.5 Hz, 12H), 1.17-1.14 (m, 1H), 1.05 (dt, J=2.9, 7.4 Hz, 1H), 0.47-0.38 (m, 1H)

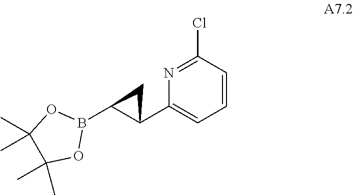 [SMILES: CC1(C)OB([C@H]2C[C@@H]2c2cccc(Cl)n2)OC1(C)C]

2-(2-fluoro-5-((1S,2S)-2-(4,4,5,5-tetramethyl-1,3,2-dioxaborolan-2-yl)cyclopropyl)phenyl)propan-2-ol (A7.3). A7.3 was prepared analogously to 7.1, using 2-(5-bromo-2-fluorophenyl)propan-2-ol. 1HNMR (400 MHz, DMSO-d6) δ=7.65-7.43 (m, 1H), 7.40-7.23 (m, 1H), 7.02-6.84 (m, 2H), 5.20 (br s, 1H), 2.02-1.88 (m, 1H), 1.44 (s, 6H), 1.18 (d, J=3.3 Hz, 5H), 1.05-0.79 (m, 2H), 0.15-−0.07 (m, 1H)

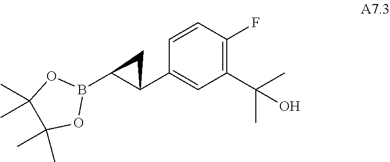 [SMILES: CC(C)(O)c1cc([C@H]2C[C@@H]2B2OC(C)(C)C(C)(C)O2)ccc1F]

1-(tert-butyl)-4-((1S,2S)-2-(4,4,5,5-tetramethyl-1,3,2-dioxaborolan-2-yl)cyclopropyl)-1H-pyrazole (A7.4) A7.4 was prepared analogously to 7.1, using 4-bromo-1-(tert-butyl)-1H-pyrazole. ¹H NMR (400 MHz, DMSO-d₆) δ=7.58 (s, 1H), 7.19 (s, 1H), 1.78 (td, J=5.4, 8.0 Hz, 1H), 1.45 (s, 9H), 1.17 (s, 12H), 0.90-0.80 (m, 2H), 0.00-−0.06 (m, 1H)

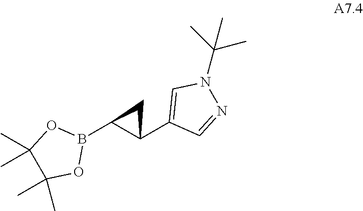 [SMILES: CC(C)(C)n1cc([C@H]2C[C@@H]2B2OC(C)(C)C(C)(C)O2)cn1]

### Preparation of methyl (1S,2S)-2-(trifluoro-4-boraneyl)cyclopropane-1-carboxylate Potassium Salt (A8.1)

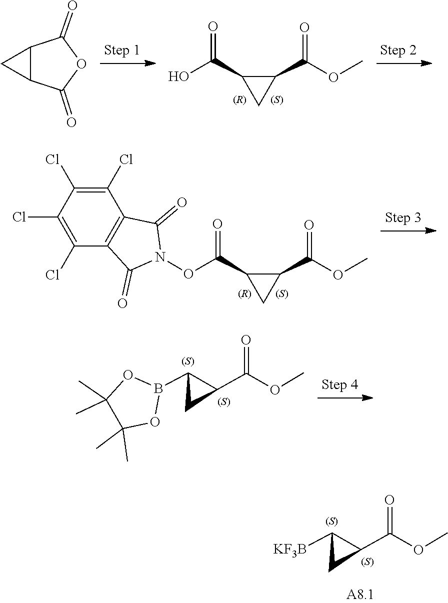

Step 1. A reactor was charged with 3-oxabicyclo[3.1.0]hexane-2,4-dione (5.00 kg, 44.6 mol), and tert-butyl methyl ether (3500 L). 1-((1R,2R)-2-(dimethylamino)cyclohexyl)-3-phenylthiourea (1.24 kg, 4.46 mol) was then added and the mixture was sparged with N₂. MeOH (18 L, 446 mol) was added, and the mixture was allowed to stir for 36 hrs at ambient temperature. Na₂CO₃ (sat. aq., 50 L) and NaCl (sat. aq., 100 L) were added, and the mixture extracted with DCM (3×50 L). The aqueous layer was then acidified to a pH of 1 with HCl (1N), and was extracted with EtOAc (5×20 L). The combined organics were washed with NaCl (sat. aq., 20 L), and concentrated to provide (1R,2S)-2-(methoxycarbonyl)cyclopropane-1-carboxylic acid.

Step 2. A reactor was charged with (1R,2S)-2-(methoxycarbonyl)cyclopropane-1-carboxylic acid (1.20 kg, 8.33 mol), 4,5,6,7-tetrachloro-2-hydroxyisoindoline-1,3-dione (2.63 kg, 8.74 mol), and DCM (24 L). DIC (1.42 L, 9.16 mol) was then added and the mixture allowed to stir for 16 hrs at ambient temperature. Water (15 L) was added, and the mixture filtered. The filtrate was portioned, and the organic layer was concentrated. The resulting solids were triturated with 1:10 DCM:EtOAc (20 L) and DCM (20 L) to obtain crude 1-methyl 2-(4,5,6,7-tetrachloro-1,3-dioxoisoindolin-2-yl) (1S,2R)-cyclopropane-1,2-dicarboxylate.

Step 3. 1-methyl 2-(4,5,6,7-tetrachloro-1,3-dioxoisoindolin-2-yl) (1S,2R)-cyclopropane-1,2-dicarboxylate (5.30 kg) was split into 13 parallel reactions: A flask was charged with 1-methyl 2-(4,5,6,7-tetrachloro-1,3-dioxoisoindolin-2-yl) (1S,2R)-cyclopropane-1,2-dicarboxylate (400 g, 937 mmol), PhCF₃ (2.8 L), followed by B₂pin₂ (476 g, 1.87 mol), and ethyl isonicotinate (21.2 g, 141 mmol). The mixture was heated to 30° C. for 16 hrs. The parallel reactions were combined, concentrated, and subjected to flash column chromatography (SiO₂, petroleum ether-EtOAc) to yield methyl (1S,2S)-2-(4,4,5,5-tetramethyl-1,3,2-dioxaborolan-2-yl)cyclopropane-1-carboxylate.

Step 4. (1S,2S)-2-(4,4,5,5-tetramethyl-1,3,2-dioxaborolan-2-yl)cyclopropane-1-carboxylate was split into 7 parallel reactions: A flask was charged with (1S,2S)-2-(4,4,5,5-tetramethyl-1,3,2-dioxaborolan-2-yl)cyclopropane-1-carboxylate (300 g, 1.33 mol), MeOH (1.5 L) and water (300 mL), followed by KHF₂ (363 g, 4.64 mol). The mixture was stirred at ambient temperature for 16 hrs. The parallel reactions were combined, filtered, and concentrated. The solids were redissolved in acetone (5 L) and filtered. The filtrate was concentrated. MTBE (10 L) was added and the mixture was stirred at ambient temperature for 30 min, before being filtered. The collected solids were then resuspended in EtOAc (10 L) and stir an additional 30 min. The solids were filtered and dried under vacuum to obtain methyl (1S,2S)-2-(trifluoro-λ⁴-boraneyl)cyclopropane-1-carboxylate potassium salt (A8.1).

### Preparation of 3-chloro-5-((1S,2S)-2-(4,4,5,5-tetramethyl-1,3,2-dioxaborolan-2-yl)cyclopropyl)pyridine (A9.1)

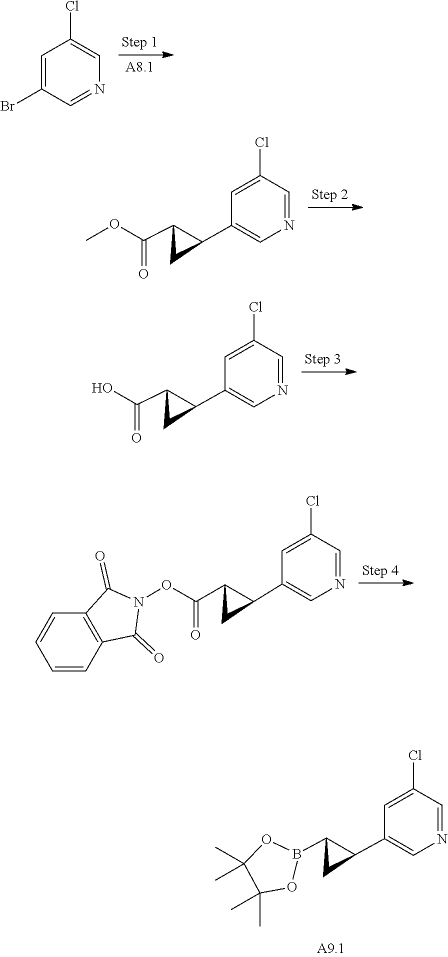 [SMILES: Clc1cncc(Br)c1 ; COC(=O)[C@H]1C[C@@H]1c1cncc(Cl)c1 ; O=C(O)[C@H]1C[C@@H]1c1cncc(Cl)c1 ; O=C(ON1C(=O)c2ccccc2C1=O)[C@H]1C[C@@H]1c1cncc(Cl)c1 ; CC1(C)OB([C@H]2C[C@@H]2c2cncc(Cl)c2)OC1(C)C]

Step 1: A flask with charged with 3-bromo-5-chloro-pyridine (6.00 g, 31.2 mmol), methyl (1S,2S)-2-(trifluoro-λ⁴-boraneyl)cyclopropane-1-carboxylate potassium salt (7.71 g, 37.4 mmol), CataCXium Pd G4 (2.31 g, 3.12 mmol), and cesium carbonate (30.5 g, 93.5 mmol). To the flask, 1,4-dioxane (120 mL) and H₂O (30 mL) were added, and the slurry was sparged with Ar for 10 minutes. A condenser was equipped, the mixture was placed under a flow of N₂, and heated to 110° C. and stirred for 18 hr. After cooling to room temperate, the mixture was concentrated under reduced pressure. The crude mixture, EtOAc (100 mL) and sat. aq. NaHCO₃ (200 mL) were added, and the mixture was transferred to a separatory funnel. The layers were separated, and the aqueous layer was extracted with EtOAc (3×100 mL). The organic extracts were combined, dried with MgSO₄, filtered and concentrated. The residue was subjected to flash column chromatography (SiO₂, hexanes-EtOAc) to yield methyl (1S,2S)-2-(5-chloro-3-pyridyl)cyclopropanecarboxylate.

Step 2: A flask was charged with methyl (1S,2S)-2-(5-chloro-3-pyridyl)cyclopropanecarboxylate (9.28 g, 43.8 mmol) followed by LiOH (2 M, 65.7 mL, 131 mmol), THF (130 mL), and MeOH (130 mL). The flask was heated to 50° C. for 1 hr. After cooling to room temperature, HCl (1 M, 136 mL, 136 mmol) was added to the flask. The mixture was transferred to a separatory funnel and then further diluted with H₂O (150 mL) and EtOAc (200 mL). The layers were separated and the aqueous was extracted with EtOAc 4× (100 mL). The organic extracts were combined, dried with Na₂SO₄, filtered, and concentrated to yield (1S,2S)-2-(5-chloro-3-pyridyl)cyclopropanecarboxylic acid that was used without further purification.

Step 3: A flask was charged with (1S,2S)-2-(5-chloro-3-pyridyl)cyclopropanecarboxylic acid (0.266 g, 1.35 mmol) and N-Hydroxyphthalimide (0.242 g, 1.48 mmol). To flask, DCM (200 mL) was added followed by N,N′-Diisopropylcarbodiimide (0.187 g, 1.48 mmol). The flask was placed under a flow of N₂ and stirred at room temperature for 18 hr. The mixture was filtered over celite, which was washed with EtOAc. The mixture was cooled to room temperature and concentrated. The residue was subjected to flash column chromatography (SiO₂, hexanes-EtOAc) to yield (1,3-dioxoisoindolin-2-yl)(1S,2S)-2-(5-chloro-3-pyridyl)cyclopropanecarboxylate,

Step 4: A flask was charged with (1,3-dioxoisoindolin-2-yl) (1S,2S)-2-(5-chloro-3-pyridyl)cyclopropanecarboxylate (1.36 g, 3.97 mmol) and bis(pinacoloato)diboron (2.02 g, 7.94 mmol). To the flask, PhCF₃ (23 mL) was added and the solution was sparged with argon for 10 minutes, then tert-butyl isonicotinate (0.36 g, 1.98 mmol) was added by syringe. The mixture was stirred for 18 hr at 30° C. The mixture was cooled to room temperature and concentrated. The residue was subject flash column chromatography (SiO₂, hexanes-EtOAc) to yield 3-chloro-5-((1S,2S)-2-(4,4,5,5-tetramethyl-1,3,2-dioxaborolan-2-yl)cyclopropyl)pyridine (A9.1).

2-((1S,2S)-2-(4-fluorophenyl)cyclopropyl)-4,4,5,5-tetramethyl-1,3,2-dioxaborolane (A9.2). A9.2 was prepared following the procedures described in Steps 3 and 4 in the synthesis of A9.1, starting with commercially-available (1S,2S)-2-(4-fluorophenyl)cyclopropane-1-carboxylic acid. ES/MS: m/z 263.3 [M+H]⁺.

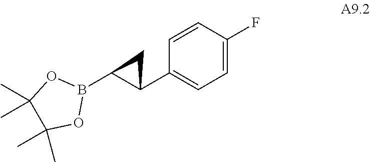 [SMILES: CC1(C)OB([C@H]2C[C@@H]2c2ccc(F)cc2)OC1(C)C]

2-((1R,2R)-2-(4-fluorophenyl)cyclopropyl)-4,4,5,5-tetramethyl-1,3,2-dioxaborolane (A9.3). A9.3 was prepared following the procedures described in Steps 3 and 4 in the synthesis of A9.1, starting with commercially-available (1R,2R)-2-(4-fluorophenyl)cyclopropane-1-carboxylic acid. ¹H NMR (400 MHz, Chloroform-d) δ 7.03 (dd, J=8.6, 5.5 Hz, 2H), 6.92 (t, J=8.7 Hz, 2H), 2.08 (dt, J=8.2, 5.4 Hz, 1H), 1.25 (s, 6H), 1.24 (s, 6H), 1.14 (ddd, J=8.3, 6.8, 3.7 Hz, 1H), 0.94 (ddd, J=9.3, 5.3, 3.8 Hz, 1H), 0.23 (ddd, J=9.9, 6.7, 5.6 Hz, 1H).

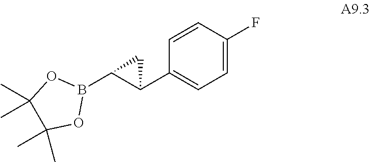 [SMILES: CC1(C)OB([C@@H]2C[C@H]2c2ccc(F)cc2)OC1(C)C]

2-(2-(3,4-difluorophenyl)-2-methylcyclopropyl)-4,4,5,5-tetramethyl-1,3,2-dioxaborolane (A9.4). A9.2 was prepared following the procedures described in Steps 3 and 4 in the synthesis of A9.1, starting with commercially-available 2-(3,4-difluorophenyl)-2-methylcyclopropane-1-carboxylic acid. ES/MS: m/z 263.3 [M+H]⁺.

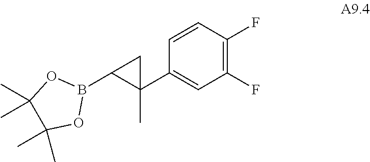 [SMILES: CC1(c2ccc(F)c(F)c2)CC1B1OC(C)(C)C(C)(C)O1]

2-(2′,3′-dihydrospiro[cyclopropane-1,1′-inden]-2-yl)-4,4,5,5-tetramethyl-1,3,2-dioxaborolane (A9.5). A9.5 was prepared following the procedures described in Steps 3 and 4 in the synthesis of A9.1, starting with commercially-available 2′,3′-dihydrospiro[cyclopropane-1,1′-indene]-2-carboxylic acid.

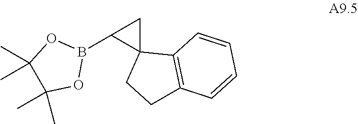 [SMILES: CC1(C)OB(C2CC23CCc2ccccc23)OC1(C)C]

3-(difluoromethyl)-5-((1S,2S)-2-(4,4,5,5-tetramethyl-1,3,2-dioxaborolan-2-yl)cyclopropyl)pyridine (A9.6). A9.6 was prepared analogously to A9.1, using 3-bromo-5-(difluoromethyl)pyridine; in Step 1, RuPhos Pd G3 was used instead of cataCXium Pd G4. ¹H NMR (400 MHz, DMSO-d₆) δ 8.03 (s, 1H), 7.68 (t, J=59.4 Hz, 1H), 7.61 (s, 1H), 1.94-1.83 (m, 1H), 1.19 (s, 6H), 1.18 (s, 6H), 0.99-0.86 (m, 2H), 0.17-0.01 (m, 1H).

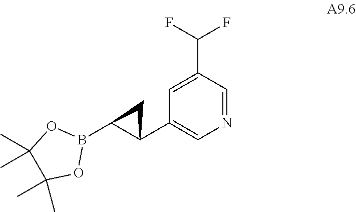 [SMILES: CC1(C)OB([C@H]2C[C@@H]2c2cncc(C(F)F)c2)OC1(C)C]

1-(difluoromethyl)-4-((1S,2S)-2-(4,4,5,5-tetramethyl-1,3,2-dioxaborolan-2-yl)cyclopropyl)-1H-pyrazole (A9.7). A9.7 was prepared analogously to A9.1, using 4-bromo-1-(difluoromethyl)-1H-pyrazole; in Step 1, RuPhos Pd G3 was used instead of cataCXium Pd G4.

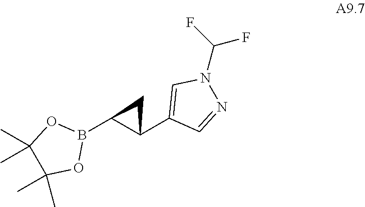 [SMILES: CC1(C)OB([C@H]2C[C@@H]2c2cnn(C(F)F)c2)OC1(C)C]

3-fluoro-5-((1S,2S)-2-(4,4,5,5-tetramethyl-1,3,2-dioxaborolan-2-yl)cyclopropyl)pyridine (A9.8). A9.8 was prepared analogously to A9.1, using 3-bromo-5-fluoropyridine; in Step 1, RuPhos Pd G3 was used instead of cataCXium Pd G4.

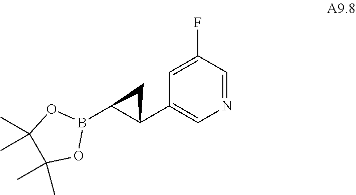 [SMILES: CC1(C)OB([C@H]2C[C@@H]2c2cncc(F)c2)OC1(C)C]

rac-1-((1S,2S)-2-(4,4,5,5-tetramethyl-1,3,2-dioxaborolan-2-yl)cyclopropyl)-1H-indazole (A9.9). A9.9 was prepared from commercially-available ethyl rac-(1S,2S)-2-(1H-indazol-1-yl)cyclopropane-1-carboxylate via procedures described in Steps 2-4 of the synthesis of A9.1.

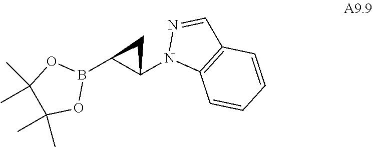 [SMILES: CC1(C)OB([C@H]2C[C@@H]2n2ncc3ccccc32)OC1(C)C]

2-(methyl-d3)-4-((1S,2S)-2-(4,4,5,5-tetramethyl-1,3,2-dioxaborolan-2-yl)cyclopropyl)-2H-1,2,3-triazole (A9.10). A9.10 was prepared following the procedures described in the synthesis of A9.1, starting with A13.2 and using methyl (1S,2S)-2-(4,4,5,5-tetramethyl-1,3,2-dioxaborolan-2-yl)cyclopropane-1-carboxylate (Step 3 in synthesis of A8.1) instead of A8.1 in Step 1.

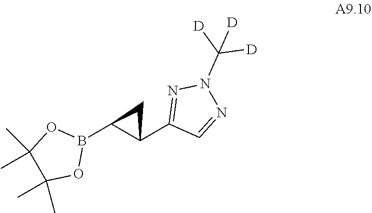 [SMILES: [2H]C([2H])([2H])n1ncc([C@H]2C[C@@H]2B2OC(C)(C)C(C)(C)O2)n1]

1-(methyl-d3)-4-((1S,2S)-2-(4,4,5,5-tetramethyl-1,3,2-dioxaborolan-2-yl)cyclopropyl)-1H-pyrazole (A9.11). A9.11 was prepared following the procedures described in the synthesis of A9.1, starting with A13.5 and using methyl (1S,2S)-2-(4,4,5,5-tetramethyl-1,3,2-dioxaborolan-2-yl)cyclopropane-1-carboxylate (Step 3 in synthesis of A8.1) instead of A8.1 in Step 1.

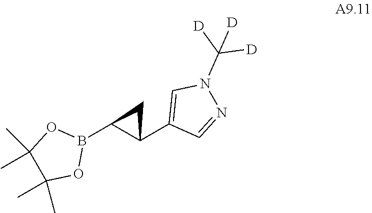 [SMILES: [2H]C([2H])([2H])n1cc([C@H]2C[C@@H]2B2OC(C)(C)C(C)(C)O2)cn1]

1-(methyl-d₃)-3-((1S,2S)-2-(4,4,5,5-tetramethyl-1,3,2-dioxaborolan-2-yl)cyclopropyl)-1H-pyrazole (A9.12). A9.12 was prepared following the procedures described in the synthesis of A9.1, starting with A13.6 and using methyl (1S,2S)-2-(4,4,5,5-tetramethyl-1,3,2-dioxaborolan-2-yl)cyclopropane-1-carboxylate (Step 3 in synthesis of A8.1) instead of A8.1 in Step 1.

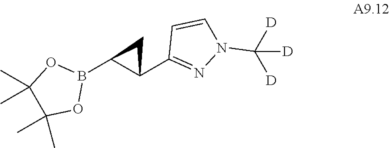 [SMILES: [2H]C([2H])([2H])n1ccc([C@H]2C[C@@H]2B2OC(C)(C)C(C)(C)O2)n1]

4-(methyl-d3)-2-((1S,2S)-2-(4,4,5,5-tetramethyl-1,3,2-dioxaborolan-2-yl)cyclopropyl)pyrimidine (A9.13). A9.13 was prepared following the procedures described in the synthesis of A9.1, starting with A13.3 and using methyl (1S,2S)-2-(4,4,5,5-tetramethyl-1,3,2-dioxaborolan-2-yl)cyclopropane-1-carboxylate (Step 3 in synthesis of A8.1) instead of A8.1 in Step 1.

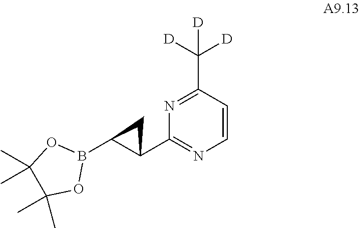 [SMILES: [2H]C([2H])([2H])c1ccnc([C@H]2C[C@@H]2B2OC(C)(C)C(C)(C)O2)n1]

3-fluoro-2-((1S,2S)-2-(4,4,5,5-tetramethyl-1,3,2-dioxaborolan-2-yl)cyclopropyl)pyridine (A9.14). A9.14 was prepared following the procedures described in the synthesis of A9.1, starting with 2-bromo-3-fluoropyridine and using methyl (1S,2S)-2-(4,4,5,5-tetramethyl-1,3,2-dioxaborolan-2-yl)cyclopropane-1-carboxylate (Step 3 in synthesis of A8.1) instead of A8.1 in Step 1.

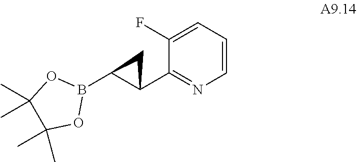 [SMILES: CC1(C)OB([C@H]2C[C@@H]2c2ncccc2F)OC1(C)C]

5-fluoro-2-((1S,2S)-2-(4,4,5,5-tetramethyl-1,3,2-dioxaborolan-2-yl)cyclopropyl)pyrimidine (A9.15). A9.15 was prepared following the procedures described in the synthesis of A9.1, starting with 2-bromo-5-fluoropyrimidine and using methyl (1S,2S)-2-(4,4,5,5-tetramethyl-1,3,2-dioxaborolan-2-yl)cyclopropane-1-carboxylate (Step 3 in synthesis of A8.1) instead of A8.1 in Step 1.

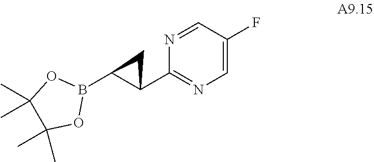 [SMILES: CC1(C)OB([C@H]2C[C@@H]2c2ncc(F)cn2)OC1(C)C]

5-fluoro-1-(methyl-d3)-4-((1S,2S)-2-(4,4,5,5-tetramethyl-1,3,2-dioxaborolan-2-yl)cyclopropyl)-1H-pyrazole (A9.16). A9.16 was prepared following the procedures described in the synthesis of A9.1, starting with A13.7 and using methyl (1S,2S)-2-(4,4,5,5-tetramethyl-1,3,2-dioxaborolan-2-yl)cyclopropane-1-carboxylate (Step 3 in synthesis of A8.1) instead of A8.1 in Step 1.

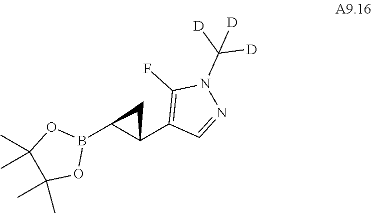 [SMILES: [2H]C([2H])([2H])n1ncc([C@H]2C[C@@H]2B2OC(C)(C)C(C)(C)O2)c1F]

### Preparation of trifluoro((1S,2S)-2-(4-fluorophenyl)cyclopropyl)-λ⁴-borane, Potassium Salt (A10.1)

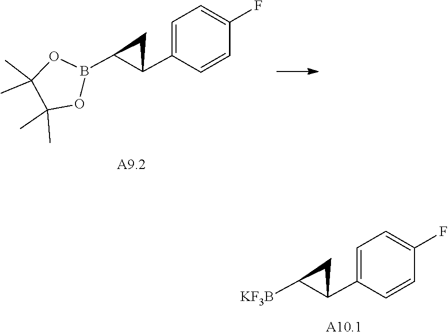

A mixture of 2-((1S,2S)-2-(4-fluorophenyl)cyclopropyl)-4,4,5,5-tetramethyl-1,3,2-dioxaborolane (A9.2, 0.758 g, 2.89 mmol) and potassium bifluoride (1.56 g, 20.2 mmol) in MeOH (12 mL) and water (2.4 mL) was stirred at rt for 16 h. The mixture was concentrated. To the residue was added MeCN (30 mL) and the slurry was stirred at rt for 3 h. The solution was decanted and filtered. The remaining solid was stirred with another portion of MeCN (30 mL) for 30 min. The mixture was filtered. The combined filtrates were concentrated to afford trifluoro((1S,2S)-2-(4-fluorophenyl)cyclopropyl)-λ⁴-borane, potassium salt (10.1).

rac-trifluoro((1S,2S)-2-(4-fluorophenyl)cyclopropyl)-λ⁴-borane, potassium salt (A10.2). A10.2 was prepared analogously to A10.1, using A1.2.

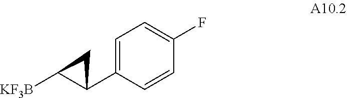

trifluoro((1R,2R)-2-(4-fluorophenyl)cyclopropyl)-λ⁴-borane, potassium salt (A10.3). A10.3 was prepared analogously to A10.1, using A9.3.

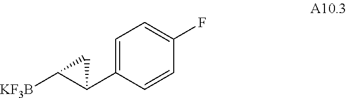

rac-trifluoro((1S,2S)-2-(3-methoxyphenyl)cyclopropyl)-λ4-borane, potassium salt (A10.4). A10.4 was prepared analogously to A10.1, using A1.1.

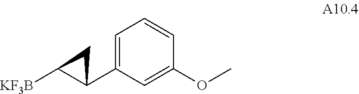

rac-((1S,2S)-2-(2,2-difluorobenzo[d][1,3]dioxol-5-yl)cyclopropyl)trifluoro-4-borane, potassium salt (A10.5). A10.5 was prepared analogously to A10.1, using A1.3.

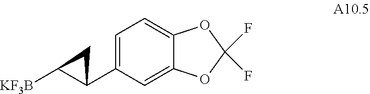

rac-trifluoro((1S,2S)-2-(2,3,4-trifluorophenyl)cyclopropyl)-λ4-borane, potassium salt (A10.6). A10.6 was prepared analogously to A10.1, using A1.5.

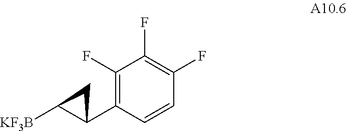

rac-2-((1S,2S)-2-(trifluoro-λ⁴-boraneyl)cyclopropyl)pyridine, potassium salt (A10.7). A10.7 was prepared analogously to A10.1, using A1.7.

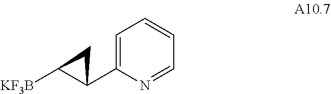

rac-2-fluoro-4-((1S,2S)-2-(trifluoro-λ⁴-boraneyl)cyclopropyl)benzonitrile, potassium salt (A10.8). A10.8 was prepared analogously to A10.1, using A1.9.

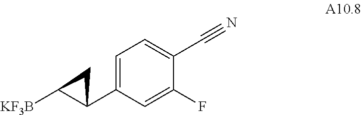

rac-4-((1S,2S)-2-(trifluoro-λ⁴-boraneyl)cyclopropyl)benzonitrile, potassium salt (A10.9).

A10.9 was prepared analogously to A10.1, using A1.10.

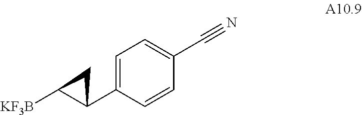

rac-trifluoro((1S,2S)-2-(2,3,6-trifluorophenyl)cyclopropyl)-λ⁴-borane, potassium salt (A10.10). A10.10 was prepared analogously to A10.1, using A1.23.

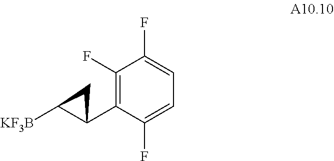

rac-trifluoro((1S,2S)-2-(4-fluoro-3-(trifluoromethyl)phenyl)cyclopropyl)-λ⁴-borane, potassium salt (A10.11). A10.11 was prepared analogously to A10.1, using A1.24.

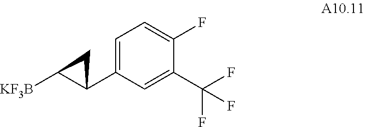

4-((1S,2S)-2-(trifluoro-λ⁴-boraneyl)cyclopropyl)-2-(trifluoromethyl)pyrimidine, potassium salt (A10.12). A10.12 was prepared analogously to A10.1, using A1.25.

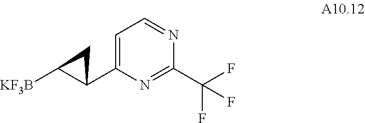

4-((1R,2R)-2-(trifluoro-λ⁴-boraneyl)cyclopropyl)-2-(trifluoromethyl)pyrimidine, potassium salt (A10.13). A10.13 was prepared analogously to A10.1, using A1.26.

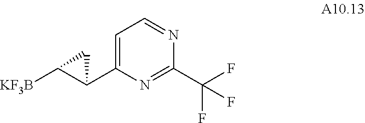

rac-1-(difluoromethyl)-3-((1S,2S)-2-(trifluoro-λ⁴-boraneyl)cyclopropyl)-1H-pyrazole, potassium salt (A10.14). A10.14 was prepared analogously to A10.1, using A1.27.

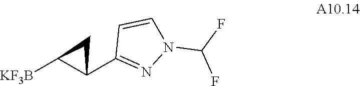

4-fluoro-3-((1S,2S)-2-(trifluoro-λ4-boraneyl)cyclopropyl)benzonitrile, potassium salt (A10.15). A10.15 was prepared analogously to A10.1, using A1.28.

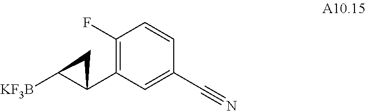

rac-trifluoro((1S,2S)-2-(2-fluorophenyl)cyclopropyl)-λ4-borane, potassium salt (A10.16). A10.16 was prepared analogously to A10.1, using A1.29.

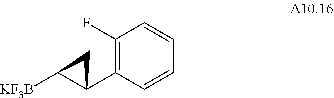

rac-trifluoro((1S,2S)-2-(3-fluorophenyl)cyclopropyl)-λ4-borane, potassium salt (A10.17). A10.17 was prepared analogously to A10.1, using A1.30.

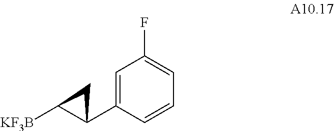

rac-3-((1S,2S)-2-(trifluoro-4-boraneyl)cyclopropyl)benzonitrile, potassium salt (A10.18).

A10.18 was prepared analogously to A10.1, using A1.31.

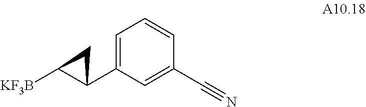

rac-3-fluoro-5-((1S,2S)-2-(trifluoro-λ4-boraneyl)cyclopropyl)benzonitrile, potassium salt (A10.19). A10.19 was prepared analogously to A10.1, using A1.32.

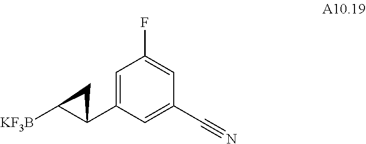

4-((1S,2S)-2-(trifluoro-λ⁴-boraneyl)cyclopropyl)-1-(trifluoromethyl)-1H-pyrazole, potassium salt (A10.20). A10.20 was prepared analogously to A10.1, using A1.33.

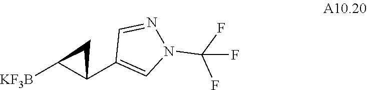

4-((1R,2R)-2-(trifluoro-λ⁴-boraneyl)cyclopropyl)-1-(trifluoromethyl)-1H-pyrazole, potassium salt (A10.21). A10.21 was prepared analogously to A10.1, using A1.34.

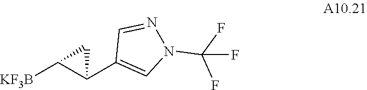

rac-((1S,2S)-2-(4-chloro-3-fluorophenyl)cyclopropyl)trifluoro-1-borane, potassium salt (A10.22). A10.22 was prepared analogously to A10.1, using A1.39.

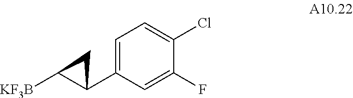

rac-((1S,2S)-2-(2,6-difluoro phenyl)cyclopropyl)trifluoro-λ⁴-borane, potassium salt (A10.23). A10.23 was prepared analogously to A10.1, using A1.40.

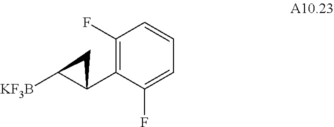

((1S,2S)-2-(3,4-difluorophenyl)cyclopropyl)trifluoro-λ4-borane, potassium salt (A10.24). A10.24 was prepared analogously to A10.1, using A6.1.

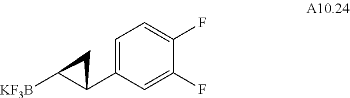

((1S,2S)-2-(2,4-difluorophenyl)cyclopropyl)trifluoro-λ4-borane, potassium salt (A10.25). A10.25 was prepared analogously to A10.1, using A6.2.

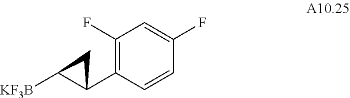

(2-(3,4-difluorophenyl)-2-methylcyclopropyl)trifluoro-λ⁴-borane, potassium salt (A10.26). A10.26 was prepared analogously to A10.1, using A9.4.

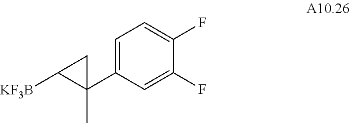

2-cyclopropyl-6-((1S,2S)-2-(trifluoro-λ⁴-boraneyl)cyclopropyl)pyridine, potassium salt (A10.27). A10.27 was prepared analogously to A10.1, using A5.2.

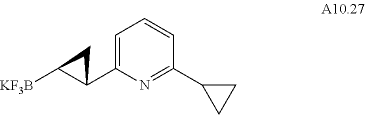

2-methyl-4-((1S,2S)-2-(trifluoro-λ⁴-boraneyl)cyclopropyl)thiazole, potassium salt (A10.28). A10.28 was prepared analogously to A10.1, using A5.1.

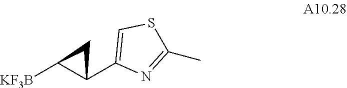

rac-((1S,2R)-2-benzylcyclopropyl)trifluoro-λ⁴-borane, potassium salt (A10.29). A10.29 was prepared analogously to A10.1, using A3.1.

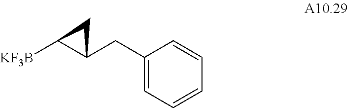

trifluoro((1S,2S)-2-phenylcyclopropyl)-λ⁴-borane, potassium salt (A10.30). A10.30 was prepared analogously to A10.1, using A5.3.

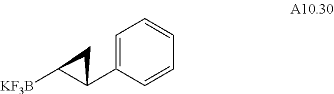

(2′,3′-dihydrospiro[cyclopropane-1,1′-inden]-2-yl)trifluoro-λ⁴-borane, potassium salt (A10.31). A10.31 was prepared analogously to A10.1, using A9.5.

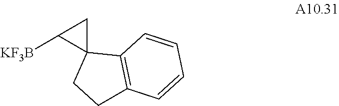

3-fluoro-5-((1S,2S)-2-(trifluoro-λ⁴-boraneyl)cyclopropyl)pyridine, potassium salt (A10.32). A10.32 was prepared analogously to A10.1, using A9.8. The product was purified by trituration with EtOAc/hexanes.

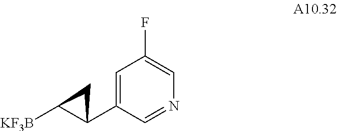

rac-1-((1S,2S)-2-(trifluoro-λ⁴-boraneyl)cyclopropyl)-1H-indazole, potassium salt (A10.33). A10.33 was prepared analogously to A10.1, using A9.9.

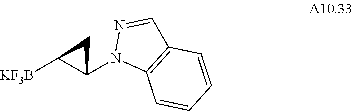

3-chloro-5-((1S,2S)-2-(trifluoro-λ⁴-boraneyl)cyclopropyl)pyridine, potassium salt (A10.34). A10.33 was prepared analogously to A10.1, using A9.8. The product was purified by trituration with EtOAc/hexanes.

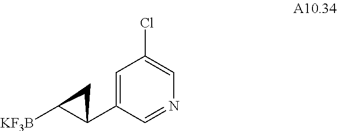

### Preparation of trifluoro((1S,2S)-2-(3-(trifluoromethoxy)phenyl)cyclopropyl)-λ⁴-borane, Potassium Salt (A11.1)

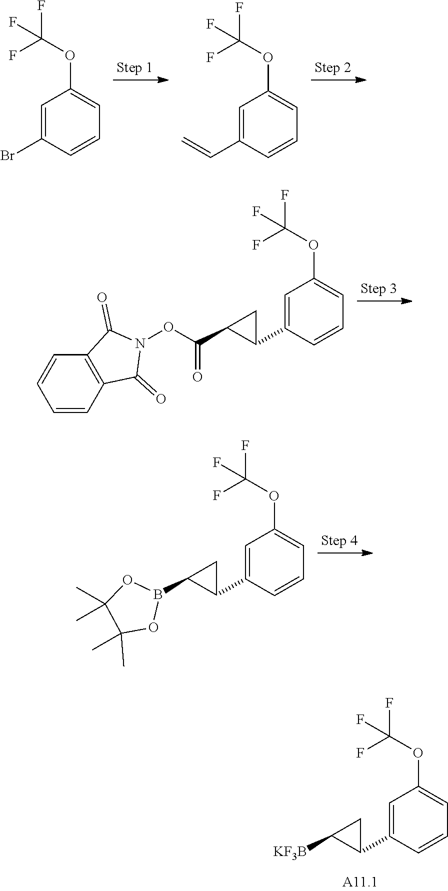

Step 1. To a solution of 1-bromo-3-(trifluoromethoxy)benzene (5.0 g, 20.7 mmol, 3.0 mL) in dioxane (50 mL), H₂O (10 mL) was added 4,4,5,5-tetramethyl-2-vinyl-1,3,2-dioxaborolane (5.5 g, 41.4 mmol), Pd(dppf)Cl₂ (3.0 g, 4.1 mmol), and Cs₂CO₃ (13.5 g, 41.4 mmol). The mixture was stirred at 100° C. for 4 h under nitrogen. After cooling to room temperature, the reaction mixture was diluted with water and extracted with ethyl acetate. The combined organics were washed with brine, dried, and concentrated under reduced pressure. The resulting residue was purified via silica gel column chromatography (0-15% ethyl acetate in petroleum ether) to afford 1-(trifluoromethoxy)-3-vinylbenzene.

Step 2. To a solution of 1-(trifluoromethoxy)-3-vinylbenzene (860.0 mg, 4.5 mmol) in DCM (6.0 mL) at 0° C. was (R)-Pheox Ru(II) (29.1 mg, 45.7 μmol) followed by 1,3-dioxoisoindolin-2-yl 2-diazoacetate (0.2 M, 20.5 mL). The reaction mixture was stirred at 0° C. for 4 h. The reaction mixture was concentrated under reduced pressure and the resulting residue was purified via silica gel column chromatography (0-10% ethyl acetate in petroleum ether) to afford to afford 1,3-dioxoisoindolin-2-yl (1S,2S)-2-(3-(trifluoromethoxy)phenyl)cyclopropane-1-carboxylate. 1H NMR (400 MHz, CDCl₃) δ=7.97-7.87 (m, 2H), 7.86-7.76 (m, 2H), 7.43-7.29 (m, 1H), 7.19-7.08 (m, 2H), 7.02 (s, 1H), 2.80 (ddd, J=4.3, 6.8, 9.4 Hz, 1H), 2.32-2.18 (m, 1H), 1.93-1.81 (m, 1H), 1.63 (ddd, J=5.1, 6.8, 8.5 Hz, 1H)

Step 3. A solution of 1,3-dioxoisoindolin-2-yl (1S,2S)-2-(3-(trifluoromethoxy)phenyl)cyclopropane-1-carboxylate (1.3 g, 3.4 mmol) in trifluoromethylbenzene (19.9 mL) was degassed and purged with N₂ for three times. t-butyl isonicotinate (304.5 mg, 1.7 mmol, 0.5 eq) and bis(pinacolato)diboron (1.7 g, 6.8 mmol) were added into the solution. The mixture was stirred at 30° C. for 16 h under N₂ atmosphere. The reaction mixture was concentrated under reduced pressure and the resulting residue was purified via silica gel column chromatography (0-10% ethyl acetate in petroleum ether) to afford 4,4,5,5-tetramethyl-2-((1S,2S)-2-(3-(trifluoromethoxy)phenyl)cyclopropyl)-1,3,2-dioxaborolane.

1H NMR (400 MHz, CDCl₃) δ=7.26-7.20 (m, 1H), 7.03-6.97 (m, 2H), 6.93-6.89 (m, 1H), 2.11 (td, J=5.4, 8.0 Hz, 1H), 1.26 (d, J=4.0 Hz, 12H), 1.01 (d dd, J=3.9, 5.3, 9.7 Hz, 1H), 0.93-0.83 (m, 1H), 0.37-0.27 (m, 1H)

Step 4. To a solution of 4,4,5,5-tetramethyl-2-((1S,2S)-2-(3-(trifluoromethoxy)phenyl)cyclopropyl)-1,3,2-dioxaborolane (870 mg, 2.6 mmol) in MeOH (13.0 mL), H₂O (2.6 mL), was added KHF₂ (1.4 g, 18.5 mmol). The reaction mixture was stirred at 20° C. for 16 h. The reaction mixture was concentrated under reduced pressure and to the resulting residue was added MeCN (15 mL). The mixture was shaken vigorously, and the MeCN layer was decanted. An additional portion of MeCN (12 mL) was added and this procedure was repeated. The combined MeCN layers were concentrated to give a residue. The residue was add deionized water (15 mL) and lyophilized to afford trifluoro((1S,2S)-2-(3-(trifluoromethoxy)phenyl)cyclopropyl)-λ⁴-borane, potassium salt A11.1.

1H NMR (400 MHz, DMSO-d6) δ=7.26 (t, J=7.9 Hz, 1H), 6.95 (dd, J=1.1, 7.9 Hz, 2H), 6.84 (s, 1H), 1.56-1.46 (m, 1H), 0.69 (dt, J=2.6, 7.3 Hz, 1H), 0.46-0.33 (m, 1H), −0.18-−0.32 (m, 1H).

5-chloro-3-fluoro-2-((1S,2S)-2-(trifluoro-λ⁴-boraneyl)cyclopropyl)pyridine, potassium salt (A11.2) A11.2 was prepared analogously to A11.1, beginning with 5-chloro-3-fluoro-2-bromopyridine. 1H NMR (400 MHz, DMSO-d6) δ=8.25 (d, J=1.6 Hz, 1H), 7.79 (dd, J=2.0, 10.0 Hz, 1H), 1.87-1.80 (m, 1H), 0.82 (br dd, J=2.8, 9.3 Hz, 1H), 0.70 (t, J=7.3 Hz, 1H), 0.18-0.03 (m, 1H)

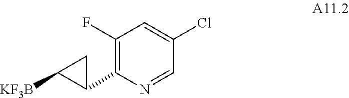

((1S,2S)-2-(3-chloro-4-fluorophenyl)cyclopropyl)trifluoro-M-borane, potassium salt (A11.3) A11.3 was prepared analogously to A11.1, beginning with 2-chloro-1-fluoro-4-bromobenzene. 1H NMR (400 MHz, DMSO-d6) δ=7.21-7.10 (m, 1H), 7.06 (dd, J=2.3, 7.3 Hz, 1H), 6.91 (ddd, J=2.3, 4.8, 8.5 Hz, 1H), 1.53-1.37 (m, 1H), 0.71-0.56 (m, 1H), 0.42-0.30 (m, 1H), −0.24-−0.37 (m, 1H)

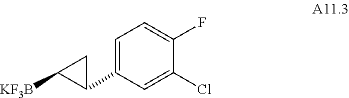

((1S,2S)-2-(2,5-difluorophenyl)cyclopropyl)trifluoro-M-borane, potassium salt (A11.4) A11.4 was prepared analogously to A11.1, beginning with 1,4-difluoro-2-bromobenzene. 1H NMR (400 MHz, DMSO-d6) δ=7.05 (dt, J=4.9, 9.4 Hz, 1H), 6.85-6.77 (m, 1H), 6.53 (ddd, J=3.2, 6.3, 10.0 Hz, 1H), 1.66 (br d, J=5.4 Hz, 1H), 0.68 (dt, J=2.4, 7.4 Hz, 1H), 0.49-0.39 (m, 1H), −0.16-−0.29 (m, 1H)

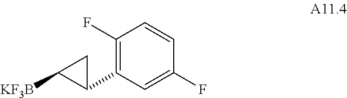

### 2-methoxy-6-((1S,2S)-2-(trifluoro-M-boraneyl)cyclopropyl)pyridine, Potassium Salt (A11.5)

A11.5 was prepared analogously to A11.1, beginning with 2-bromo-6-methoxypyridine. 1H NMR (400 MHz, DMSO-d6) δ=7.40 (t, J=7.7 Hz, 1H), 6.62 (d, J=7.4 Hz, 1H), 6.36 (d, J=7.9 Hz, 1H), 3.75 (s, 3H), 1.64-1.43 (m, 1H), 0.67 (br dd, J=3.0, 9.8 Hz, 1H), 0.61-0.53 (m, 1H), 0.02 (tt, J=3.5, 9.5 Hz, 1H)

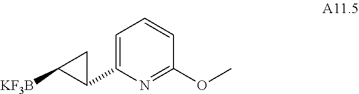

1-methyl-3-((1S,2S)-2-(trifluoro-4-boraneyl)cyclopropyl)-1H-pyrazole, potassium salt (A11.6) A11.6 was prepared analogously to A11.1, beginning with 3-iodo-1-methyl-1H-pyrazole. 1H NMR (400 MHz, DMSO-d6) δ=7.35 (d, J=2.0 Hz, 1H), 5.63 (d, J=2.1 Hz, 1H), 3.67 (s, 3H), 1.42-1.34 (m, 1H), 0.42 (dt, J=1.8, 7.3 Hz, 1H), 0.29-0.22 (m, 1H), −0.38-−0.43 (m, 1H)

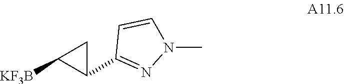

((1S,2S)-2-(3,5-difluorophenyl)cyclopropyl)trifluoro-M-borane, potassium salt (A11.7) A11.7 was prepared analogously to A11.1, beginning with 1-bromo-3,5-difluorobenzene. 1H NMR (400 MHz, DMSO-d6) δ=6.76 (tt, J=2.4, 9.4 Hz, 1H), 6.66-6.54 (m, 2H), 1.55-1.44 (m, 1H), 0.68 (dt, J=2.8, 7.3 Hz, 1H), 0.49-0.38 (m, 1H), −0.16-−0.30 (m, 1H)

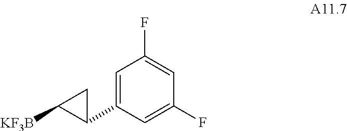

((1S,2S)-2-(4-chloro-2-fluorophenyl)cyclopropyl)trifluoro-M-borane, potassium salt (A11.8) A11.8 was prepared analogously to A11.1, beginning with 1-bromo-4-chloro-2-fluoro-benzene. 1HNMR (400 MHz, CHLOROFORM-d) δ=7.25-7.15 (m, 1H), 7.06 (dd, J=2.1, 8.4 Hz, 1H), 6.79 (t, J=8.5 Hz, 1H), 1.68-1.58 (m, 1H), 0.66 (dt, J=2.6, 7.3 Hz, 1H), 0.40 (br d, J=10.0 Hz, 1H), −0.14-−0.30 (m, 1H)

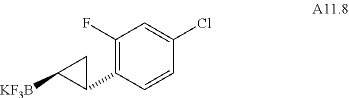

((1S,2S)-2-(2-chlorophenyl)cyclopropyl)trifluoro-M-borane, potassium salt (A11.9) A11.9 was prepared analogously to steps 2-4 in the preparation of A11.1, beginning with 1-chloro-2-vinylbenzene. 1H NMR (400 MHz, DMSO-d6) δ=7.28 (dd, J=1.2, 8.0 Hz, 1H), 7.20-7.08 (m, 1H), 7.01 (dt, J=1.6, 7.6 Hz, 1H), 6.77 (dd, J=1.5, 7.8 Hz, 1H), 1.90-1.78 (m, 1H), 0.69 (dt, J=2.4, 7.3 Hz, 1H), 0.32 (br d, J=10.1 Hz, 1H), −0.14-−0.25 (m, 1H)

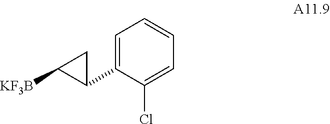

trifluoro((1S,2S)-2-(3-(trifluoromethyl)phenyl)cyclopropyl)-λ4-borane, potassium salt (A11.10) A11.10 was prepared analogously to steps 2-4 in the preparation of A11.1, beginning with 1-(trifluoromethyl)-3-vinylbenzene. 1H NMR (400 MHz, DMSO-d6) δ=7.41-7.31 (m, 2H), 7.24-7.18 (m, 2H), 1.58-1.52 (m, 1H), 0.70 (dt, J=2.4, 7.3 Hz, 1H), 0.45 (br d, J=10.1 Hz, 1H), −0.19-−0.28 (m, 1H)

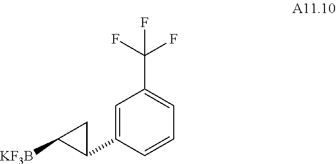

trifluoro((1S,2S)-2-(4-(trifluoromethyl)phenyl)cyclopropyl)-λ4-borane, potassium salt (A11.11) A11.11 was prepared analogously to steps 2-4 in the preparation of A11.1, beginning with 1-(trifluoromethyl)-4-vinylbenzene. 1H NMR (400 MHz, DMSO-d6) δ=6.64 (d, J=8.3 Hz, 2H), 6.30 (d, J=8.1 Hz, 2H), 0.79-0.65 (m, 1H), −0.09 (dt, J=2.5, 7.4 Hz, 1H), −0.35 (br d, J=10.1 Hz, 1H), −0.93-−1.09 m, 1H)

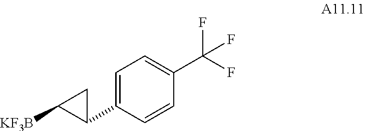

### trifluoro((1S,2S)-2-(o-tolyl)cyclopropyl)-λ4-borane, Potassium Salt (A11.12)

A11.12 was prepared analogously to steps 2-4 in the preparation of A11.1, beginning with 1-methyl-2-vinyl-benzene. 1HNMR (400 MHz, DMSO-d6) δ=7.05-6.94 (m, 2H), 6.94-6.88 (m, 1H), 6.77 (d, J=7.6 Hz, 1H), 2.32 (s, 3H), 1.50-1.42 (m, 1H), 0.53 (dt, J=2.3, 7.1 Hz, 1H), 0.40-0.30 (m, 1H)

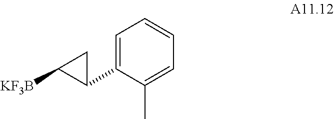

((1S,2S)-2-(2,6-difluorophenyl)cyclopropyl)trifluoro-M-borane, potassium salt (A11.13) A11.13 was prepared analogously to steps 2-4 in the preparation of A11.1, beginning with 1,3-difluoro-2-vinyl-benzene. 1H NMR (400 MHz, DMSO-d6) δ=7.11-7.01 (m, 1H), 6.94-6.81 (m, 2H), 1.59-1.46 (m, 1H), 0.82 (br dd, J=1.1, 8.8 Hz, 1H), 0.51 (br t, J=6.9 Hz, 1H), 0.16-0.07 (m, 1H)

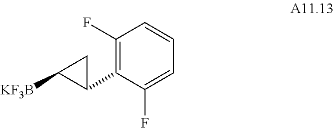

((1S,2S)-2-(3-chlorophenyl)cyclopropyl)trifluoro-M-borane, potassium salt (A11.14) A11.14 was prepared analogously to steps 2-4 in the preparation of A11.1, beginning with 1-chloro-3-vinyl-benzene. 1HNMR (400 MHz, DMSO-d₆) δ=7.20-7.11 (m, 1H), 7.03 (ddd, J=0.9, 2.0, 7.9 Hz, 1H), 6.95-6.85 (m, 2H), 1.56-1.35 (m, 1H), 0.66 (dt, J=2.6, 7.3 Hz, 1H), 0.48-0.30 (m, 1H), −0.17-−0.37 (m, 1H)

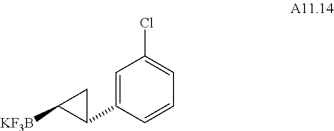

((1S,2S)-2-(4-chlorophenyl)cyclopropyl)trifluoro-λ⁴-borane, potassium salt (A11.15) A11.15 was prepared analogously to steps 2-4 in the preparation of A11.1, beginning with 1-chloro-4-vinyl-benzene. 1HNMR (400 MHz, DMSO-d6) δ=7.20-7.11 (m, 2H), 6.96-6.91 (m, 2H), 1.47-1.40 (m, 1H), 0.63 (dt, J=2.5, 7.3 Hz, 1H), 0.38-0.30 (m, 1H), −0.25-−0.35 (m, 1H)

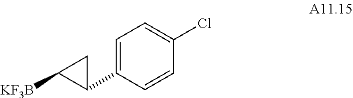

### Preparation of ((1S,2S)-2-(4-cyclopropylphenyl)cyclopropyl)trifluoro-λ⁴-borane, Potassium Salt (A12.1)

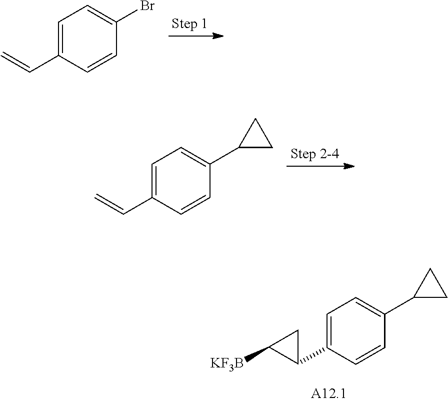

Step 1. To a solution of 1-bromo-4-vinyl-benzene (2 g, 10.9 mmol, 1.4 mL) in THF (20 mL) was added Ni(dppf)Cl₂ (149.5 mg, 218.5 μmol). The reaction mixture was degassed and purged with N₂ for three times. To the reaction mixture was added bromo(cyclopropyl)magnesium (0.5 M, 32.8 mL) dropwise at 0° C. The reaction mixture was heated at 50° C. for 8 hours. After cooling to rt, the reaction mixture was quenched with saturated aqueous NH₄Cl (50 mL) and extracted with ethyl acetate (50 mL×2). The combined organics were washed with brine (50 mL), dried, filtered and concentrated under reduced pressure. The resulting residue was purified via silica gel column chromatography (0-6% ethyl acetate in petroleum ether) to afford 1-cyclopropyl-4-vinylbenzene (A12.1).

¹H NMR (400 MHz, CHLOROFORM-d) δ=7.31 (br d, J=8.1 Hz, 2H), 7.04 (br d, J=8.0 Hz, 2H), 6.69 (dd, J=10.9, 17.6 Hz, 1H), 5.69 (d, J=17.6 Hz, 1H), 5.18 (d, J=10.9 Hz, 1H), 1.98-1.82 (m, 1H), 1.04-0.90 (m, 2H), 0.78-0.65 (m, 2H)

Steps 2-4. Steps 2-4 in the procedure of A11.1 were followed, beginning with 1-cyclopropyl-4-vinylbenzene. 1HNMR (400 MHz, DMSO-d6) δ=6.87-6.82 (m, 2H), 6.81-6.76 (m, 2H), 1.85-1.74 (m, 1H), 1.42-1.33 (m, 1H), 0.90-0.81 (m, 2H), 0.61-0.49 (m, 3H), 0.27 (br d, J=9.8 Hz, 1H), −0.30-−0.41 (m, 1H)

### Preparation of 3-bromo-2-(difluoromethyl)-5-fluoro-pyridine (A13.1)

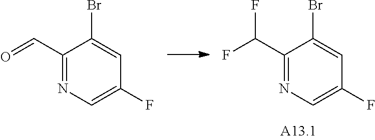 [SMILES: O=Cc1ncc(F)cc1Br ; Fc1cnc(C(F)F)c(Br)c1]

To a solution of 3-bromo-5-fluoro-pyridine-2-carbaldehyde (100 mg, 0.49 mmol) in DCM (5 mL) at 0° C. was added diethylaminosulfur trifluoride (0.1 mL, 0.74 mmol) dropwise over 5 min. The reaction was allowed to warm to room temp and stirred for 2 hr. A saturated sodium bicarbonate solution was added cautiously, and the aqueous layer was washed with DCM three times. The combined organic layers were dried over Na2SO4, filtered and concentrated. The residue was purified by silica chromatography (eluent: 0-100% EtOAc in hexanes) to give 3-bromo-2-(difluoromethyl)-5-fluoro-pyridine (A13.1). 1H NMR (400 MHz, Chloroform-d) δ 8.51 (d, J=2.4 Hz, 1H), 7.76 (ddt, J=7.5, 2.5, 0.9 Hz, 1H), 6.88 (t, J=53.8 Hz, 1H).

### Preparation of 4-bromo-2-(methyl-d3)-2H-1,2,3-triazole (A13.2)

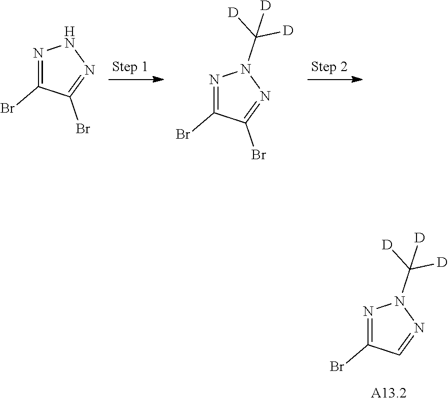 [SMILES: Brc1n[nH]nc1Br ; [2H]C([2H])([2H])n1nc(Br)c(Br)n1 ; [2H]C([2H])([2H])n1ncc(Br)n1]

Step 1. A reactor was charged with 4,5-dibromo-2H-1,2,3-triazole (500 g, 2.20 mol), K₂CO₃ (609 g, 4.4 mol), and DMF (3.0 L). The mixture was cooled to −5° C., and CD₃I (351 g, 2.42 mol) was added slowly. The mixture stirred for 3 his, and LiCl (1M, 500 ml) was added. The mixture was extracted with EtOAc (3×1 L). The combined organics were dried over Na₂SO₄, filtered, and concentrated. The mixture was subject flash column chromatography (SiO₂, petroleum ether-EtOAc) to give 4,5-dibromo-2-(methyl-d3)-2H-1,2,3-triazole. ¹³C NMR (100 MHz, CDCl₃): δ 124.2, 42.4 (septet).

Step 2. A reactor was charged with 4,5-dibromo-2-(methyl-d3)-2H-1,2,3-triazole (100 g, 410 mmol) and THF (600 mL). The mixture was cooled to −5° C., and iPrMgCl (2M in THF, 253 g, 1.23 mol) was added dropwise. The mixture stirred for 2 hrs. NH₄Cl (2M, 500 mL) was added and the mixture was extracted with MBTE (3×1 L). The combined organics were dried over Na₂SO₄, filtered, and concentrated. The residue was stirred in MeCN (25 mL) at −20° C. for 5 min and was filtered. The filtrate was concentrated to deliver 4-bromo-2-(methyl-d3)-2H-1,2,3-triazole. 1H NMR (400 MHz, CD₃Cl): δ 7.52 (s, 1H).

### 4-bromo-2-(methyl-d3)pyrimidine (A13.3)

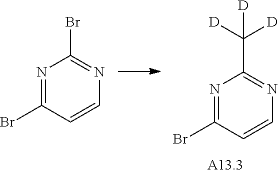 [SMILES: Brc1ccnc(Br)n1 ; [2H]C([2H])([2H])c1nccc(Br)n1]

A flask was charged with 2,4-dibromopyrimidine (8.16 g, 34 mmol), Iron(III) acetylacetonate (3.64 g, 10.3 mmol), and THF (100 mL). The mixture was cooled to 0° C. and (methyl-d3)magnesium iodide (1.0 M in ether, 52 mL, 52 mmol) was added dropwise. The mixture was allowed to warm to room temperature over 1 hr. NH₄Cl (sat. aq., 100 mL) was added and the mixture was extracted with EtOAc (3×50 mL). The combined organics were dried over Na₂SO₄, filtered, and concentrated. The resulting residue was subject to flash column chromatography (SiO₂, Hexanes-EtOAc) and the product was recrystallized from boiling hexane to deliver 4-bromo-2-(methyl-d3)pyrimidine. ¹H NMR (400 MHz, CDCl₃) δ 8.41 (d, J=5.0 Hz, 1H), 7.16 (d, J=5.0 Hz, 1H).

2-bromo-6-(methyl-d3)pyridine (A13.4). A13.4 was prepared analogously to A13.3, using 2,6-dibromopyridine instead of 2,4-dibromopyrimidine. ¹H NMR (400 MHz, CDCl₃) δ 7.42 (t, J=7.7 Hz, 1H), 7.29 (dd, J=7.9, 0.9 Hz, 1H), 7.10 (dd, J=7.5, 0.9 Hz, 1H).

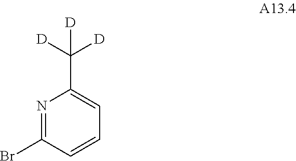 [SMILES: [2H]C([2H])([2H])c1cccc(Br)n1]

4-bromo-1-(methyl-d3)-1H-pyrazole (A13.5). A13.5 was prepared analogously to step 1 in the preparation of A13.2 using 4-bromo-1H-pyrazole instead of 4,5-dibromo-2H-1,2,3-triazole. ¹H NMR (400 MHz, CDCl3) δ 7.43 (s, 1H), 7.37 (s, 1H).

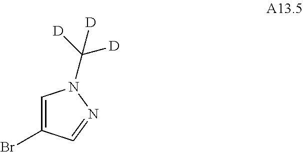

### 3-bromo-1-(methyl-d3)-1H-pyrazole (A13.6)

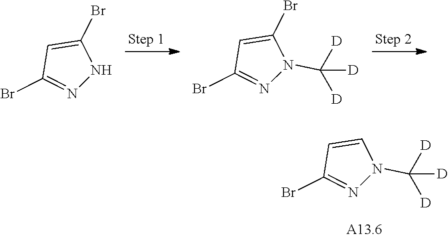

Step 1. A mixture of 3,5-dibromo-1H-pyrazole (10.0 g, 44.3 mmol), K₂CO₃ (12.2 g, 88.5 mmol) and iodomethane-d₃ (5.4 mL, 12.6 mmol) in DMF (50 mL) was stirred at rt for 16 h. The mixture was diluted with water and Et₂O. The aqueous layer was extracted with Et₂O. The combined organic layers were washed successively with brine, 10% LiCl (aq), and brine, then dried (Na₂SO₄), filtered and concentrated. The resulting residue was subjected to column chromatography on silica gel to yield 3,5-dibromo-1-(methyl-d₃)-1H-pyrazole. ES/MS: m/z 243.8 [M+H]⁺.

Step 2. To a solution of 3,5-dibromo-1-(methyl-d₃)-1H-pyrazole (10.0 g, 41.2 mmol) in THF (100 mL) at 0° C. was added i-PrMgCl (62 mL of a 2 M soln in THF, 124 mmol). The mixture was stirred at 0° C. for 1 h and then was quenched by the addition of sat. NH₄Cl solution. The mixture was extracted with Et₂O. The organic layer was washed with brine, dried (Na₂SO₄), filtered and concentrated. The residue was subjected to column chromatography on silica gel to afford 3-bromo-1-(methyl-d₃)-1H-pyrazole. ES/MS: m/z 164.0/166.0 [M+H]⁺.

### 4-bromo-5-fluoro-1-(methyl-d3)-1H-pyrazole (A13.7)

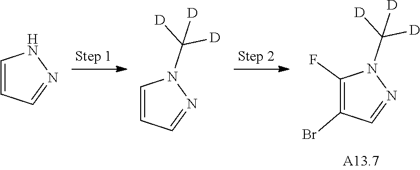 [SMILES: c1cn[nH]c1 ; [2H]C([2H])([2H])n1cccn1 ; [2H]C([2H])([2H])n1ncc(Br)c1F]

Step 1. Potassium hydroxide (7.65 g, 136 mmol) was added over 1 hr to a mixture of pyrazole (5.00 g, 76.4 mmol) in water (7.5 mL). To this solution was added d₃-iodomethane (8.5 mL, 137 mmol) dropwise over 70 minutes. The mixture stirred for 2 hr. The aqueous layer was washed twice with dichloromethane and the combined organic layers were washed with saturated sodium chloride, dried over sodium sulfate, filtered and concentrated to obtain 1-(trideuteriomethyl)pyrazole. 1H NMR (400 MHz, Chloroform-d) δ 7.49 (d, J=1.9 Hz, 1H), 7.35 (dd, J=2.2, 0.6 Hz, 1H), 6.24 (t, J=2.1 Hz, 1H).

Step 2. To a solution of 1-(trideuteriomethyl)pyrazole (3.0 g, 36 mmol) in THF (60 mL) under nitrogen at −78° C. was added dropwise a solution of nBuLi (2M in hexanes, 20 mL) over 30 min. The solution was allowed to stir at −78° C. for 30 min. Then, a solution of N-fluorobenzenesulfonimide (13.5 g 43 mmol) in THF (20 mL) was added dropwise at −78° C. over 30 min. The reaction stirred at −78° C. for 2 hr and then was allowed to warm to room temperature and stirred overnight. To the solution was added acetonitrile (60 mL) and N-bromosuccinimide (6.98 g, 392 mmol). The reaction was allowed to stir overnight. The reaction was concentrated and the residue was brought up in dichloromethane and washed three times with a saturated sodium bicarbonate solution. The organic layer was dried over sodium sulfate, filtered and concentrated. The residue was purified by silica gel chromatography (0-50% EtOAc in hex) to obtain 4-bromo-5-fluoro-1-(methyl-d3)-1H-pyrazole. 1H NMR (400 MHz, Chloroform-d) δ 7.33 (d, J=2.6 Hz, 1H).

### 5-bromo-3-fluoro-1-(methyl-d3)-1H-pyrazole (A13.8)

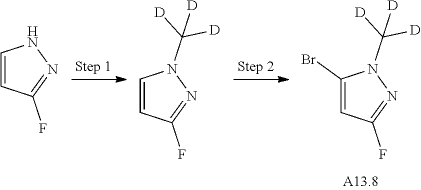 [SMILES: Fc1cc[nH]n1 ; [2H]C([2H])([2H])n1ccc(F)n1 ; [2H]C([2H])([2H])n1nc(F)cc1Br]

Step 1. To NaH (470 mg, 12.2 mmol, 60% purity) in THF (1 mL) was added 5-fluoro-1H-pyrazole (350 mg, 4.07 mmol) at 0° C. under nitrogen atmosphere. The mixture was stirred at 0° C. for 30 min. To this mixture was added d₃-iodomethane (2.36 g, 16.3 mmol) and the resulting mixture stirred at 0° C. for 1 hr. Water was added and the aqueous layer was washed with EtOAc three times. The combined organic layers were dried over Na2SO4, filtered and concentrated to give 3-fluoro-1-(trideuteriomethyl)pyrazole. 1HNMR (400 MHz, Chloroform-d) δ 7.15 (t, J=2.3 Hz, 1H), 5.74 (ddd, J=5.9, 2.3, 0.8 Hz, 1H).

Step 2. To a solution of 3-fluoro-1-(trideuteriomethyl)pyrazole (400 mg, 3.10 mmol) in THF (1 mL) at −78° C. was added dropwise a solution of n-butyl lithium (2M in hexanes, 2.33 mL, 4.66 mmol). After the solution stirred for 15 min at −78° C., bromine (0.240 mL, 4.66 mmol) was added dropwise over 5 min. The solution was allowed to stir at −30° C. for 1.5 hr. To the mixture was added saturated sodium bicarbonate and the aqueous layer was washed three times with EtOAc. The combined organic layers were dried over Na2SO4, filtered and concentrated. The residue was purified by silica gel chromatography (0-50% EtOAc in hex) to obtain 5-bromo-3-fluoro-1-(trideuteriomethyl)pyrazole. 1H NMR (400 MHz, Chloroform-d) δ 5.86 (dd, J=6.1, 1.3 Hz, 1H).

### 3-bromo-5-fluoro-1-(methyl-d3)-1H-pyrazole (A13.9)

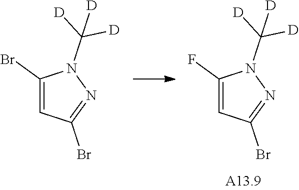 [SMILES: [2H]C([2H])([2H])n1nc(Br)cc1Br ; [2H]C([2H])([2H])n1nc(Br)cc1F]

To a solution of 3,5-dibromo-1-(trideuteriomethyl)pyrazole (1.0 g, 4.12 mmol) in THF (12 mL) under nitrogen at −78° C. was added dropwise a solution of nBuLi (2M in hexanes, 2.27 mL) over 30 min. The solution was allowed to stir at −78° C. for 30 min. Then, a solution of N-fluorobenzenesulfonimide (1.56 g, 4.94 mmol) in THF (5 mL) was added dropwise at −78° C. over 30 min. The reaction stirred at −78° C. for 2 hr and then was allowed to warm to room temperature and stirred overnight. A solution of saturated ammonium chloride was added and the mixture concentrated. The aqueous layer was washed three times with Et₂O. The combined organic layers were dried over magnesium sulfate, filtered and concentrated. The residue was purified by silica gel chromatography (0-50% EtOAc in hex) to obtain 3-bromo-5-fluoro-1-(trideuteriomethyl)pyrazole. 1H NMR (400 MHz, Chloroform-d) δ 5.80 (d, J=5.9 Hz, 1H).

### 5-bromo-4-fluoro-1-(methyl-d3)-1H-pyrazole (A13.10)

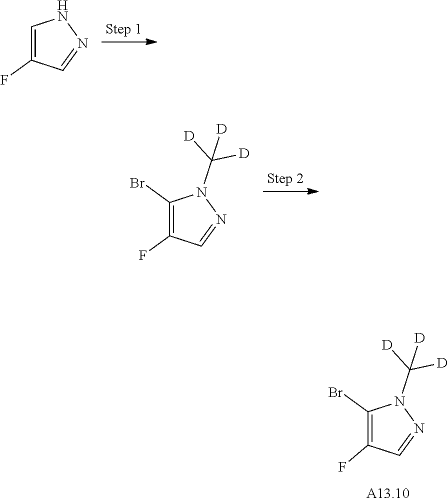 [SMILES: Fc1cn[nH]c1 ; [2H]C([2H])([2H])n1ncc(F)c1Br ; [2H]C([2H])([2H])n1ncc(F)c1Br]

Step 1. A mixture of 4-fluoro-1H-pyrazole (1.00 g, 11.6 mmol), potassium carbonate (3.21 g, 23.2 mmol) and d₃-iodomethane (3.45 g, 23.8 mmol) in DMF (12 mL) was stirred over night. The mixture was diluted with water and the aqueous layer was washed three times with Et₂O. The combined organic layers were washed with three times with 10% LiCl, dried over magnesium sulfate, filtered and concentrated to give 4-fluoro-1-(trideuteriomethyl)pyrazole. 1HNMR (400 MHz, Chloroform-d) δ 7.30 (d, J=4.2 Hz, 1H), 7.24 (dd, J=4.8, 0.8 Hz, 1H).

Step 2. To a solution of 4-fluoro-1-(trideuteriomethyl)pyrazole (684 mg, 6.63 mmol) in THF (1 mL) at −78° C. was added dropwise a solution of n-butyl lithium (2.5M in hexanes, 3.18 mL, 7.96 mmol). After the solution stirred for 15 min at −78° C., bromine (0.410 mL, 7.96 mmol) was added dropwise over 5 min. The solution was allowed to stir at −30° C. for 1.5 hr. To the mixture was added saturated sodium bicarbonate and the aqueous layer was washed three times with EtOAc. The combined organic layers were dried over Na2SO4, filtered and concentrated. The residue was purified by silica gel chromatography (0-50% EtOAc in hex) to obtain 5-bromo-4-fluoro-1-(trideuteriomethyl)pyrazole. 1H NMR (400 MHz, Chloroform-d) δ 7.37 (d, J=4.7 Hz, 1H).

### 3-bromo-4-fluoro-1-(methyl-d3)-1H-pyrazole (A13.11)

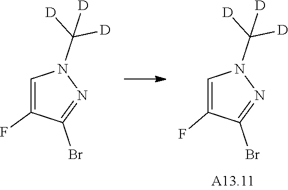 [SMILES: [2H]C([2H])([2H])n1cc(F)c(Br)n1 ; [2H]C([2H])([2H])n1cc(F)c(Br)n1]

A mixture of 3-bromo-4-fluoro-1H-pyrazole (0.507 g, 3.07 mmol), potassium carbonate (0.850 g, 6.15 mmol) and d3-iodomethane (0.913 g, 6.30 mmol) in DMF (3 mL) was stirred overnight. The mixture was diluted with water and the aqueous layer was washed three times with Et2O. The combined organic layers were washed with three times with 10% LiCl, dried over magnesium sulfate, filtered and concentrated. The residue was purified by silica gel chromatography (0-50% EtOAc in hex) to obtain 3-bromo-4-fluoro-1-(trideuteriomethyl)pyrazole. 1H NMR (400 MHz, Chloroform-d) δ 7.22 (d, J=5.2 Hz, 1H).

### 4-bromo-2-(difluoromethyl)triazole (A13.12) and 4-bromo-1-(difluoromethyl)triazole (A13.13)

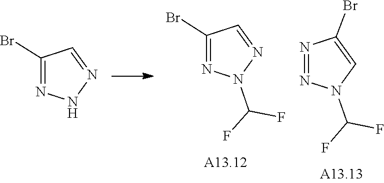

A vial was charged with 4-bromo-2H-triazole (964 mg, 6.52 mmol), sodium 2-chloro-2,2-difluoroacetate (1.99 g, 13 mmol), and cesium carbonate (6.37 g, 19.5 mmol). To this, dimethylsulfoxide (14.6 mL) was added, and the mixture was stirred and heated to 90° C. for 16 hours under a dry nitrogen atmosphere. It was allowed to cool to ambient temperature, then was poured onto water. It was extracted three times with diethyl ether, and the combined extracts were washed three times with saturated aqueous sodium chloride. It was dried over anhydrous magnesium sulfate, filtered, and concentrated. It was purified via flash chromatography (SiO₂, 5-50% diethyl ether/pentane linear gradient) to yield 4-bromo-2-(difluoromethyl)triazole (¹H NMR (400 MHz, DMSO) δ 8.45 (s, 1H), 8.16 (t, J=57.1 Hz, 1H)) as the first eluting fraction and 4-bromo-1-(difluoromethyl)triazole (¹H NMR (400 MHz, DMSO) δ 9.11 (s, 1H), 8.27 (t, J=57.9 Hz, 1H)) as the second eluting fraction. 4-bromo-2-ethyl-triazole (A13.14). A13.14 was prepared analogously to A13.2 using bromoethane instead of CD₃I. ¹H NMR (400 MHz, CDCl₃) δ 7.53 (s, 1H), 4.45 (q, J=7.4 Hz, 2H), 1.56 (t, J=7.4 Hz, 3H).

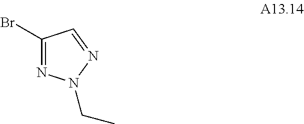

4,5-dibromo-2-propyl-triazole (A13.15). A13.15 was prepared analogously to A13.2 using 1-iodopropane instead of CD₃I. ¹H NMR (400 MHz, CDCl₃) δ 7.53 (s, 1H), 4.36 (t, J=7.0 Hz, 2H), 1.98 (h, J=7.3 Hz, 2H), 0.93 (t, J=7.4 Hz, 3H).

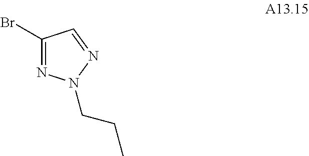

4,5-dibromo-2-isopropyl-triazole (A13.16) A13.16 was prepared analogously to A13.2 using 2-iodopropane instead of CD₃I. ¹H NMR (400 MHz, CDCl₃) δ 7.52 (s, 1H), 4.80 (hept, J=6.7 Hz, 1H), 1.57 (d, J=6.7 Hz, 7H).

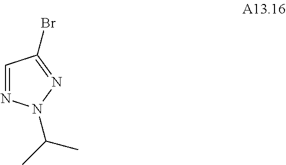

4-bromo-2-(cyclopropylmethyl)triazole (A13.17) A13.17 was prepared analogously to A13.2 using (bromomethyl)cyclopropane instead of CD₃I. ¹HNMR (400 MHz, CDCl₃) δ 7.54 (s, 1H), 4.25 (d, J=7.3 Hz, 2H), 1.44-1.34 (m, 1H), 0.67-0.59 (m, 2H), 0.44 (dt, J=6.2, 4.8 Hz, 2H).

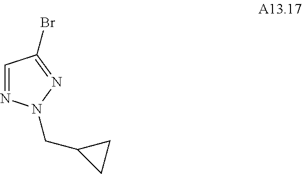

### 3-bromo-5-chloro-2-((2-(trimethylsilyl)ethoxy)methoxy)pyrazine (A13.18)

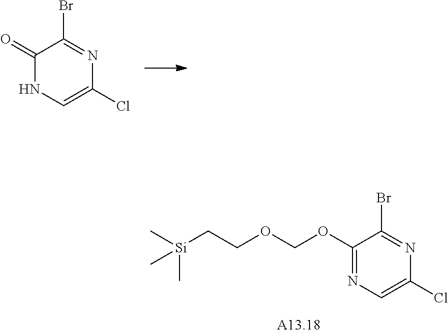 [SMILES: O=c1[nH]cc(Cl)nc1Br ; C[Si](C)(C)CCOCOc1ncc(Cl)nc1Br]

A vial was charged with 3-bromo-5-chloro-1H-pyrazin-2-one (165 mg, 0.788 mmol) and potassium carbonate (363 mg, 1.58 mmol). To this, acetone (4 mL) was added, followed by (2-((chloromethoxy)methoxy)ethyl)trimethylsilane (0.167 mL, 0.95 mmol) was added, and the mixture was allowed to stir overnight. It was filtered, concentrated, and purified via flash chromatography (SiO₂, 5-100% ethyl acetate/hexanes linear gradient) to yield the title compound. ¹H NMR (400 MHz, CDCl₃) δ 7.38 (d, J=3.4 Hz, 1H), 5.33 (d, J=4.7 Hz, 2H), 3.70-3.61 (m, 2H), 1.03-0.93 (m, 2H), 0.02 (s, 9H). 2-bromo-3-((2-(trimethylsilyl)ethoxy)methoxy)pyrazine (A13.19) A13.19 was prepared analogously to A13.18 using 3-bromo-1H-pyrazin-2-one instead of 3-bromo-5-chloro-1H-pyrazin-2-one.

¹H NMR (400 MHz, CDCl₃) δ 8.06 (d, J=2.6 Hz, 1H), 7.97 (d, J=2.6 Hz, 1H), 5.65 (s, 2H), 3.92-3.77 (m, 2H), 1.08-0.93 (m, 2H), 0.01 (s, 9H).

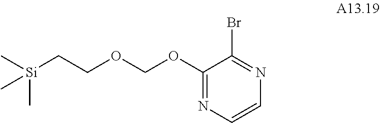 [SMILES: C[Si](C)(C)CCOCOc1nccnc1Br]

2-bromo-3-fluoro-6-((2-(trimethylsilyl)ethoxy)methoxy)pyridine (A13.20) A13.20 was prepared analogously to A13.18 using 6-bromo-5-fluoro-1H-pyridin-2-one instead of 3-bromo-5-chloro-1H-pyrazin-2-one. ¹H NMR (400 MHz, CDCl₃) δ 7.37 (dd, J=8.7, 6.9 Hz, 1H), 6.73 (dd, J=8.7, 2.8 Hz, 1H), 5.50 (s, 2H), 3.85-3.73 (m, 2H), 1.04-0.92 (m, 2H), 0.01 (s, 9H).

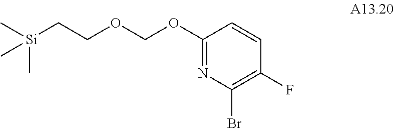 [SMILES: C[Si](C)(C)CCOCOc1ccc(F)c(Br)n1]

3-bromo-4-fluoro-2-((2-(trimethylsilyl)ethoxy)methoxy)pyridine (A13.21) A13.21 was prepared analogously to A13.18 using 3-bromo-4-fluoro-1H-pyridin-2-one one instead of 3-bromo-5-chloro-1H-pyrazin-2-one. ¹H NMR (400 MHz, CDCl₃) δ 8.05 (dd, J=8.0, 5.6 Hz, 1H), 6.75 (dd, J=7.4, 5.6 Hz, 1H), 5.64 (s, 2H), 3.89-3.74 (m, 2H), 1.03-0.93 (m, 2H), 0.01 (s, 9H).

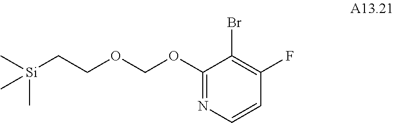 [SMILES: C[Si](C)(C)CCOCOc1nccc(F)c1Br]

3-bromo-2-fluoro-6-((2-(trimethylsilyl)ethoxy)methoxy)pyridine (A13.22) A13.22 was prepared analogously to A13.18 using 5-bromo-6-fluoro-1H-pyridin-2-one instead of 3-bromo-5-chloro-1H-pyrazin-2-one. ¹HNMR (400 MHz, CDCl₃) δ 7.80 (t, J=8.6 Hz, 1H), 6.60 (dd, J=8.4, 0.8 Hz, 1H), 5.48 (s, 2H), 3.87-3.73 (m, 2H), 1.03-0.92 (m, 2H), 0.01 (s, 9H).

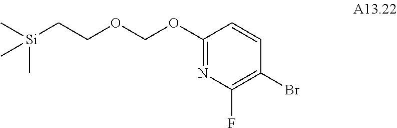 [SMILES: C[Si](C)(C)CCOCOc1ccc(Br)c(F)n1]

3-bromo-5-methyl-2-((2-(trimethylsilyl)ethoxy)methoxy)pyridine (A13.23) A13.23 was prepared analogously to A13.18 using 3-bromo-5-methyl-1H-pyridin-2-one instead of 3-bromo-5-chloro-1H-pyrazin-2-one. ¹HNMR (400 MHz, CDCl₃) δ 7.91 (dd, J=2.1, 0.9 Hz, 1H), 7.67 (dd, J=2.2, 0.7 Hz, 1H), 5.60 (s, 2H), 3.86-3.77 (m, 2H), 1.04-0.93 (m, 2H), 0.00 (s, 9H).

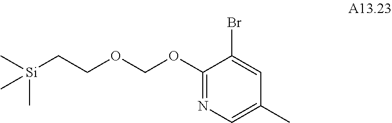

4-bromo-6-chloro-2-(((2-(trimethylsilyl)ethoxy)methoxy)methyl)pyridazin-3(2H)-one (A13.24) A13.24 was prepared analogously to A13.18 using 3-bromo-5-methyl-1H-pyridin-2-one instead of 3-bromo-5-chloro-1H-pyrazin-2-one. ¹H NMR (400 MHz, CDCl₃) δ 7.91 (dd, J=2.1, 0.9 Hz, 1H), 7.67 (dd, J=2.2, 0.7 Hz, 1H), 5.60 (s, 2H), 3.86-3.77 (m, 2H), 1.04-0.93 (m, 2H), 0.00 (s, 9H).

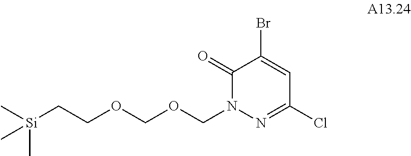 [SMILES: C[Si](C)(C)CCOCOCn1nc(Cl)cc(Br)c1=O]

### 3-bromo-2-((4-methoxybenzyl)oxy)-6-methylpyridine (A13.25)

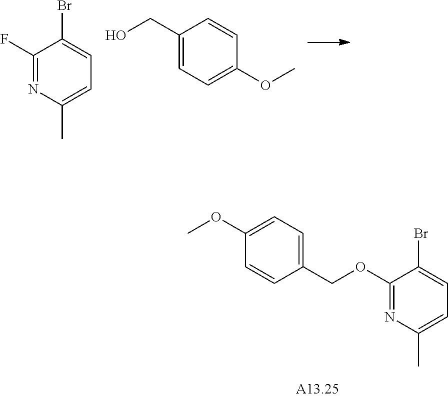

A vial was charged with (4-methoxyphenyl)methanol (392 mL, 3.16 mmol), and anhydrous THF (4.1 mL) was added. It was cooled to 0° C., and 60% sodium hydride in mineral oil (126 mg, 3.16 mmol) was added, and the mixture was stirred at this temperature for 30 min. To this, 3-bromo-2-fluoro-6-methylpyridine (400 mg, 2.11 mmol) was added in one portion, and the reaction was heated to 45° C. for 16 hours. It was cooled to ambient temperature and quenched by the addition of saturated ammonium chloride. It was extracted with ethyl acetate, and the organics were washed with saturated sodium chloride, dried over anhydrous sodium sulfate, filtered, and concentrated. It was purified via flash chromatography (SiO₂, 0-30% ethyl acetate/hexanes linear gradient) to yield the title compound. ¹H NMR (400 MHz, CDCl₃) δ 7.65 (d, J=7.7 Hz, 1H), 7.47-7.40 (m, 2H), 6.94-6.87 (m, 2H), 6.61 (d, J=7.7, 1H), 5.38 (s, 2H), 3.81 (s, 3H), 2.41 (s, 3H).

4-bromo-3-fluoro-2-((4-methoxybenzyl)oxy)pyridine (A13.25) A13.25 was prepared analogously to A13.24 using 4-bromo-2,3-difluoropyridine instead of 3-bromo-2-fluoro-6-methylpyridine. ¹H NMR (400 MHz, CDCl₃) δ 7.74 (dd, J=5.4, 0.8 Hz, 1H), 7.45-7.37 (m, 2H), 7.05 (dd, J=5.4, 4.0 Hz, 1H), 6.94-6.86 (m, 2H), 5.39 (s, 2H), 3.81 (s, 3H).

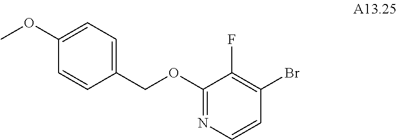 [SMILES: COc1ccc(COc2nccc(Br)c2F)cc1]

3-bromo-5-chloro-2-((4-methoxybenzyl)oxy)pyridine (A13.26) A13.26 was prepared analogously to A13.24 using 3-bromo-5-chloro-2-fluoropyridine instead of 3-bromo-2-fluoro-6-methylpyridine. H NMR (400 MHz, CDCl₃) δ 8.05 (d, J=2.3 Hz, 1H), 7.81 (d, J=2.4 Hz, 1H), 7.45-7.36 (m, 2H), 6.94-6.86 (m, 2H), 5.36 (s, 2H), 3.81 (s, 3H).

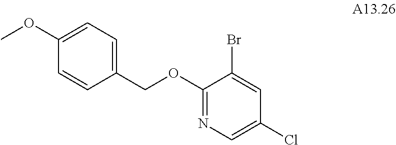 [SMILES: COc1ccc(COc2ncc(Cl)cc2Br)cc1]

### 3-bromo-5-fluoro-1-(4-methoxybenzyl)pyridin-2(1H)-one (A13.27)

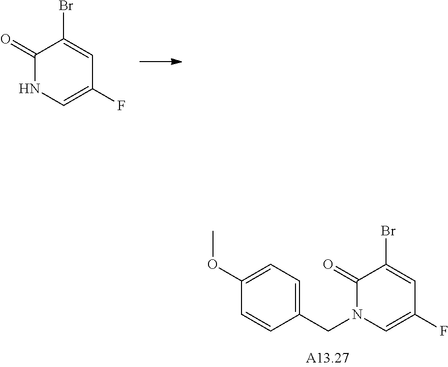 [SMILES: O=c1[nH]cc(F)cc1Br ; COc1ccc(Cn2cc(F)cc(Br)c2=O)cc1]

A vial was charged with 3-bromo-5-fluoro-1H-pyridin-2-one (300 mg, 1.56 mmol), DMF (8 mL), 1-(bromomethyl)-4-methoxybenzene (0.36 mL, 2.3 mmol), and potassium carbonate (720 mg, 3.13 mmol). It was sealed and heated to 45° C. for 16 hours. It was cooled to ambient temperature, diluted with water, and extracted twice with ethyl acetate. The combined extracts were washed with saturated sodium chloride, dried over anhydrous sodium sulfate, filtered, and concentrated. It was purified via flash chromatography (SiO₂, 5-50% ethyl acetate/hexanes linear gradient) to yield the title compound. ¹H NMR (400 MHz, CDCl₃) δ 7.69 (dd, J=6.7, 3.1 Hz, 1H), 7.31-7.27 (m, 2H), 7.19 (dd, J=4.0, 3.1 Hz, 1H), 6.94-6.85 (m, 2H), 5.07 (s, 2H), 3.81 (s, 3H).

### 4-bromo-6-chloro-2-(2-trimethylsilylethoxymethyl)pyridazin-3-one (A13.28)

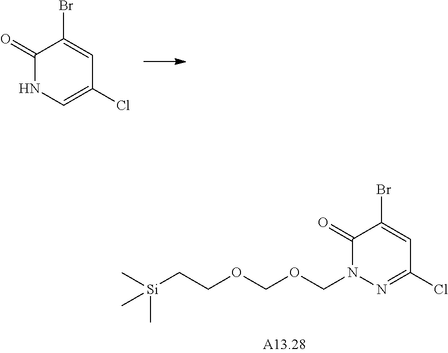 [SMILES: O=c1[nH]cc(Cl)cc1Br ; C[Si](C)(C)CCOCOCn1nc(Cl)cc(Br)c1=O]

A vial was charged with 5-bromo-3-chloro-1H-pyridazin-6-one (500 mg, 2.39 mmol) and DMF (8.4 mL), and it was cooled to 0° C. To this, 60% sodium hydride in mineral oil (115 mg, 2.86 mmol) was added, and it was stirred for 20 minutes. To this, 2-(trimethylsilyl)ethoxymethyl chloride (0.55 mL, 3.1 mmol) was added, and the mixture was allowed to warm to ambient temperature and stir overnight. It was quenched by the addition of saturated ammonium chloride, extracted with ethyl acetate, and the extract was washed with saturated sodium chloride and 1M lithium chloride. It was dried over anhydrous sodium sulfate, filtered, and concentrated. It was purified via flash chromatography (SiO₂, 5-30% ethyl acetate/hexanes linear gradient) to yield the title compound. ¹H NMR (400 MHz, CDCl₃) δ 7.62 (s, 1H), 5.46 (s, 2H), 3.78-3.69 (m, 2H), 1.02-0.92 (m, 2H), 0.01 (s, 9H).

5-bromo-2-((4-methoxybenzyl)oxy)pyridine (A13.29) A13.29 was prepared analogously to A13.24 using 5-bromo-2-fluoropyridine instead of 3-bromo-2-fluoro-6-methylpyridine.

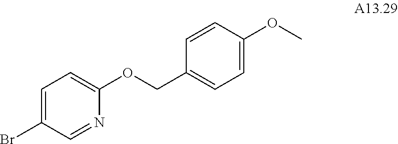 [SMILES: COc1ccc(COc2ccc(Br)cn2)cc1]

4-bromo-2-((4-methoxybenzyl)oxy)pyridine A(13.30) A13.30 was prepared analogously to A13.24 using 4-bromo-2-fluoropyridine instead of 3-bromo-2-fluoro-6-methylpyridine.

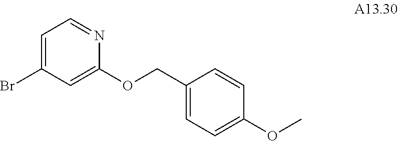 [SMILES: COc1ccc(COc2cc(Br)ccn2)cc1]

### Preparation of 2′-chloro-4-hydroxy-5′,6-dimethyl-2H-[1,4′-bipyridin]-2-one (B1.1)

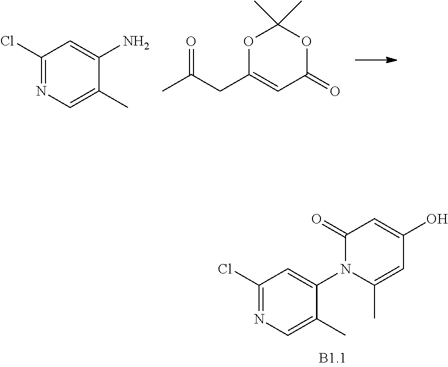

A flask was charged with 2-chloro-5-methylpyridin-4-amine (85 g, 461 mmol), 2,2-dimethyl-6-(2-oxopropyl)-4H-1,3-dioxin-4-one (79 g, 554 mmol), and 1,4-dioxane (850 mL). The mixture was heated to 90° C. for 3 hr, followed by addition of H₂SO₄ (42 mL, 77 g, 785 mmol) at 90° C. over 30 min. The mixture was allowed to stir at 90° C. for 1 hr. The mixture was concentrated and then diluted with MTBE (500 mL) and water (500 mL). The mixture stirred at ambient temperature for 30 min, and then filtered and washed with water. The filtrate was dried under vacuum to yield the title compound (B1.1). ¹H NMR (400 MHz, CDCl₃-d): δ 5.34 (s, 1H), 3.34 (s, 2H), 2.24 (s, 3H), 1.71 (s, 6H).

2′-bromo-4-hydroxy-5′,6-dimethyl-2H-[1,4′-bipyridin]-2-one (B1.2) B1.2 was prepared analogously to B1.1 using 2-bromo-5-methylpyridin-4-amine. ¹HNMR (400 MHz, DMSO-d₆): δ 11.1-10.6 (m, 1H), 7.59-7.54 (m, 1H), 7.45 (d, J=2.0 Hz, 1H), 7.36 (d, J=8.2 Hz, 1H), 5.98 (d, J=1.6 Hz, 1H), 5.62 (d, J=2.4 Hz, 1H), 1.93 (s, 3H), 1.82 (s, 3H)

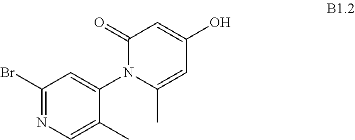 [SMILES: Cc1cnc(Br)cc1-n1c(C)cc(O)cc1=O]

2′-bromo-3′-fluoro-4-hydroxy-5′,6-dimethyl-2H-[1,4′-bipyridin]-2-one (B1.3) B1.3 was prepared analogously to B1.1 using 2-bromo-3-fluoro-5-methylpyridin-4-amine. ¹H NMR (400 MHz, DMSO-d₆) δ=11.02 (br s, 1H), 8.41 (s, 1H), 6.04 (d, J=1.5 Hz, 1H), 5.60 (d, J=2.4 Hz, 1H), 2.03 (s, 3H), 1.88 (s, 3H)

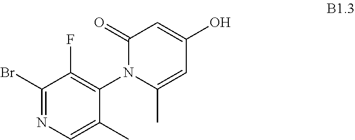 [SMILES: Cc1cnc(Br)c(F)c1-n1c(C)cc(O)cc1=O]

2′-chloro-3′-fluoro-4-hydroxy-5′,6-dimethyl-2H-[1,4′-bipyridin]-2-one (B1.4) B1.4 was prepared analogously to B1.1 using 2-chloro-3-fluoro-5-methylpyridin-4-amine.

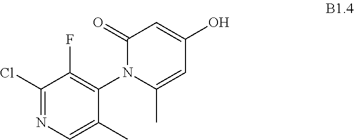 [SMILES: Cc1cnc(Cl)c(F)c1-n1c(C)cc(O)cc1=O]

2′-bromo-5′-chloro-4-hydroxy-6-methyl-2H-[1,4′-bipyridin]-2-one (B1.5) B1.5 was prepared analogously to B1.1 using 2-bromo-5-chloropyridin-4-amine.

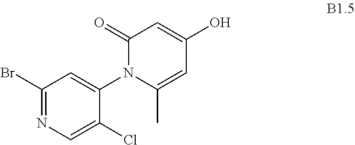 [SMILES: Cc1cc(O)cc(=O)n1-c1cc(Br)ncc1Cl]

2′-bromo-3′-chloro-4-hydroxy-6-methyl-2H-[1,4′-bipyridin]-2-one (B1.6) B1.6 was prepared analogously to B1.1 using 2-bromo-3-chloropyridin-4-amine.

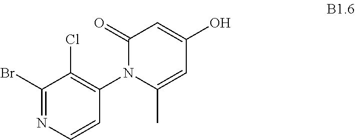 [SMILES: Cc1cc(O)cc(=O)n1-c1ccnc(Br)c1Cl]

2′-bromo-5′-chloro-3′-fluoro-4-hydroxy-6-methyl-2H-[1,4′-bipyridin]-2-one (B1.7) B1.7 was prepared analogously to B1.1 using 2-bromo-5-chloro-3-fluoropyridin-4-amine.

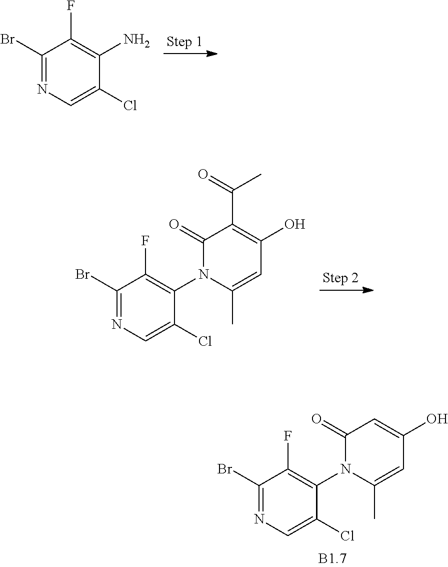 [SMILES: Nc1c(Cl)cnc(Br)c1F ; CC(=O)c1c(O)cc(C)n(-c2c(Cl)cnc(Br)c2F)c1=O ; Cc1cc(O)cc(=O)n1-c1c(Cl)cnc(Br)c1F]

Step 1. To a solution of 2-bromo-5-chloro-3-fluoropyridin-4-amine (1.5 g, 6.6 mmol) in dimethyl acetamide (15.0 mL) was added 2,2,6-trimethyl-1,3-dioxin-4-one (9.4 g, 66.5 mmol) and the mixture was stirred at 140° C. for 16 h. The reaction was diluted with water (20 mL), extracted with ethyl acetate (25 mL×3), and the combined organic phase was washed with water (25 mL×3) and brine (20 mL×3), dried over Na₂SO₄, filtered, and contracted under vacuum to give a residue. The residue was subjected to flash column chromatography (SiO2, Ethyl acetate/Petroleum ether=5˜10%) to afford 3-acetyl-2′-bromo-5′-chloro-3′-fluoro-4-hydroxy-6-methyl-2H-[1,4′-bipyridin]-2-one. 1HNMR (400 MHz, DMSO-d6) δ=8.79 (s, 1H), 6.37 (s, 1H), 2.55 (s, 3H), 2.08 (s, 3H)

Step 2. A solution of 3-acetyl-2′-bromo-5′-chloro-3′-fluoro-4-hydroxy-6-methyl-2H-[1,4′-bipyridin]-2-one (1.5 g, 3.9 mmol) in sulfuric acid (15.0 mL) was stirred at 110° C. for 5 h. The reaction mixture was cooled to room temperature, quenched by water (20 mL) and neutralized with saturated NaHCO₃ aqueous solution to pH=7. The resultant mixture was filtered and the filter cake was washed with petroleum ether (20 mL×3), dried under reduced pressure to afford 2′-bromo-5′-chloro-3′-fluoro-4-hydroxy-6-methyl-2H-[1,4′-bipyridin]-2-one. 1HNMR (400 MHz, DMSO-d6) δ=8.73 (s, 1H), 6.07 (s, 1H), 5.61 (d, J=1.6 Hz, 1H), 1.95 (s, 3H)

2′-bromo-3′-chloro-4-hydroxy-5′,6-dimethyl-2H-[1,4′-bipyridin]-2-one (B1.8) B1.8 was prepared analogously to B1.7 using 2-bromo-3-chloro-5-methylpyridin-4-amine.

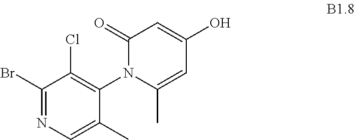 [SMILES: Cc1cnc(Br)c(Cl)c1-n1c(C)cc(O)cc1=O]

### Preparation of 2′,3-dichloro-4-hydroxy-5′,6-dimethyl-2H-[1,4′-bipyridin]-2-one (B2.1)

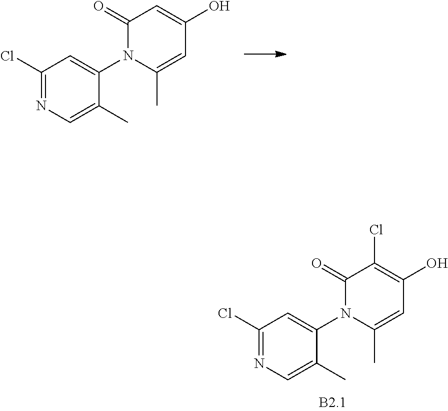 [SMILES: Cc1cnc(Cl)cc1-n1c(C)cc(O)cc1=O ; Cc1cnc(Cl)cc1-n1c(C)cc(O)c(Cl)c1=O]

A reactor was charged with 2′-chloro-4-hydroxy-5′,6-dimethyl-2H-[1,4′-bipyridin]-2-one (450 g, 1.80 mol), DCE (3 L), and 2-propanol (2.25 L). N-chlorosuccinimide (284 g, 2.12 mmol) was the added portion-wise. The mixture was stirred for 2 hours at 55° C. The mixture was then cooled to room temperature and poured into water (10 L). The mixture was filtered and the resulting solids dried under vacuum to provide crude 2′,3-dichloro-4-hydroxy-5′,6-dimethyl-2H-[1,4′-bipyridin]-2-one (B2.1), which was used without further purification.

2′-bromo-3-chloro-3′-fluoro-4-hydroxy-5′,6-dimethyl-2H-[1,4′-bipyridin]-2-one (B2.2) B2.2 was prepared analogously to B2.1 using B1.3. ¹H NMR (400 MHz, DMSO-d₆) δ=11.87 (s, 1H), 8.45 (s, 1H), 6.24 (s, 1H), 2.04 (s, 3H), 1.92 (s, 3H). Chiral Separation was performed by HPLC.

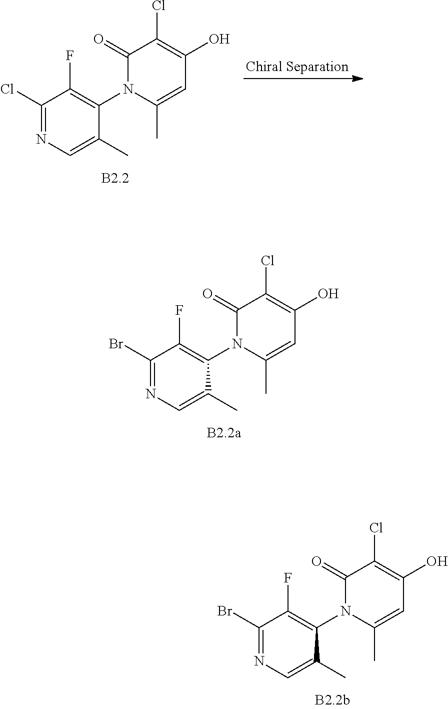

2′,3-dichloro-3′-fluoro-4-hydroxy-5′,6-dimethyl-2H-[1,4′-bipyridin]-2-one (B2.3, B2.3a, B2.3b) B2.3 was prepared analogously to B2.1 using B1.4. Atropisomers B2.3a and B2.3b were separated by HPLC (ChiralPak IC-3, 4.6×50 mm, 3 μm; MeOH, hexane, DCM 5:45:50 with 0.1% formic acid, 1 mL/min, 25° C.).

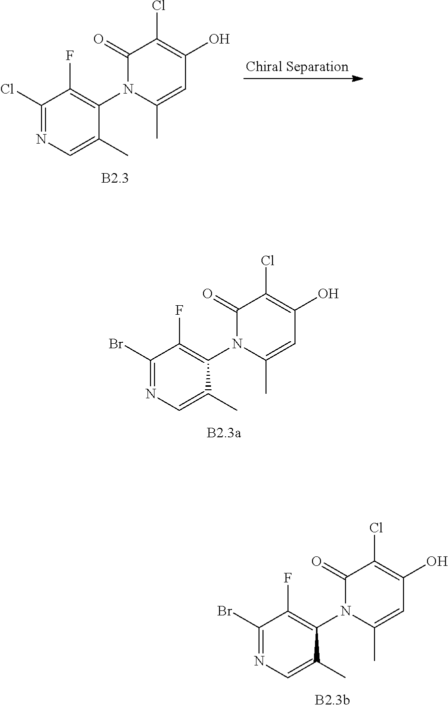

2′-bromo-3,5′-dichloro-4-hydroxy-6-methyl-2H-[1,4′-bipyridin]-2-one (B2.4) B2.4 was prepared analogously to B2.1 using B1.5.

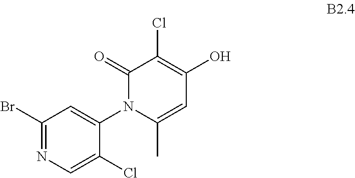 [SMILES: Cc1cc(O)c(Cl)c(=O)n1-c1cc(Br)ncc1Cl]

2′-bromo-3,3′-dichloro-4-hydroxy-6-methyl-2H-[1,4′-bipyridin]-2-one (B2.5) B2.5 was prepared analogously to B2.1 using B1.6.

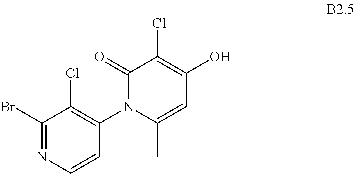 [SMILES: Cc1cc(O)c(Cl)c(=O)n1-c1ccnc(Br)c1Cl]

2′-bromo-3,5′-dichloro-3′-fluoro-4-hydroxy-6-methyl-2H-[1,4′-bipyridin]-2-one (B2.6) B2.6 was prepared analogously to B2.1 using B1.7. Chiral separation was done by SFC (column: DAICEL CHIRALCEL OZ (250*25 mm, 10 um); mobile phase: [A: CO2; B: EtOH (0.1% NH₃H₂O)]; B %: 40.00%-40.00%)

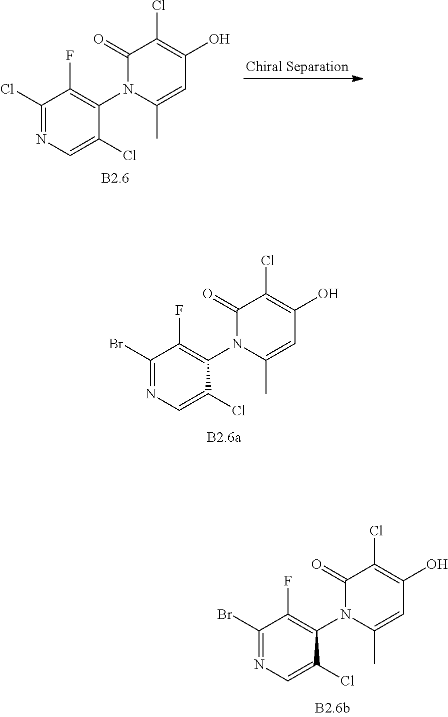

2′-bromo-3,3′-dichloro-4-hydroxy-5′,6-dimethyl-2H-[1,4′-bipyridin]-2-one (B2.7) B2.7 was prepared analogously to B2.1 using B1.7. Chiral separation was done by SFC (column: DAICEL CHIRALPAK IC (250 mm*50 mm, 10 um); mobile phase: [A: CO2; B: MeOH (0.1% IPAm)]; B %: 45.00%-45.00%)

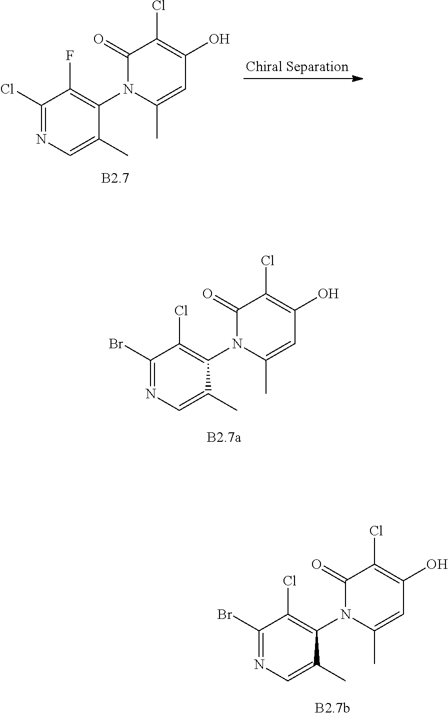

### Preparation of 2′,3-dichloro-5′,6-dimethyl-2-oxo-2H-[1,4′-bipyridin]-4-yl trifluoromethanesulfonate (B3.1, B3.1a, B3.1b)

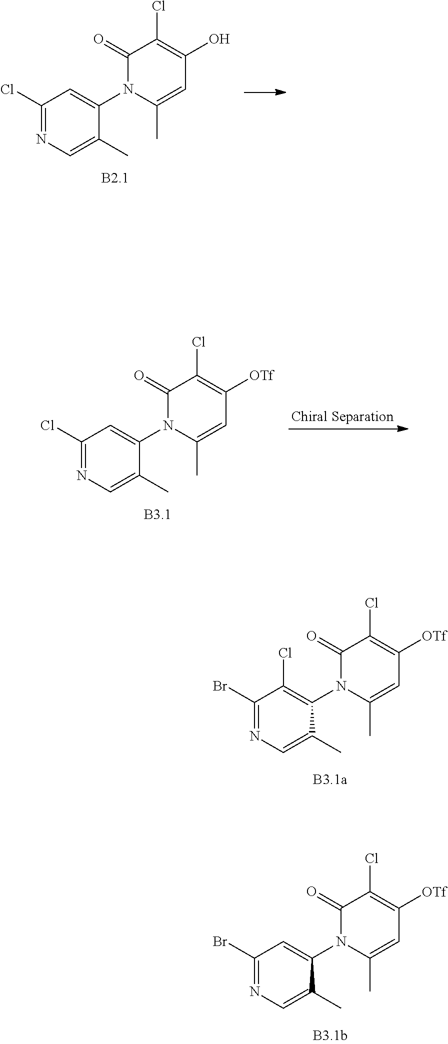

A reactor was charged with 2′,3-dichloro-4-hydroxy-5′,6-dimethyl-2H-[1,4′-bipyridin]-2-one (470 g), DCM (11.7 L), and Et₃N (500 g, 4.94 mol). The mixture was cooled to 0° C. and Tf₂O (698 g, 2.47 mol) was added slowly over 30 min. The mixture was allowed to warm to ambient temperature over 1 hr. Water (10 L) was added, and the organic layer was collected and concentrated. The residue was subject the flash column chromatography followed by preparatory HPLC to provide 2′,3-dichloro-5′,6-dimethyl-2-oxo-2H-[1,4′-bipyridin]-4-yl trifluoromethanesulfonate (B3.1). Atropisomers B3.1a and B3.1b were resolved by SFC.

2′,3-dichloro-3′-fluoro-5′,6-dimethyl-2-oxo-2H-[1,4′-bipyridin]-4-yl trifluoromethanesulfonate (B3.2a) B3.2a was prepared analogously to B3.1, using B2.3a.

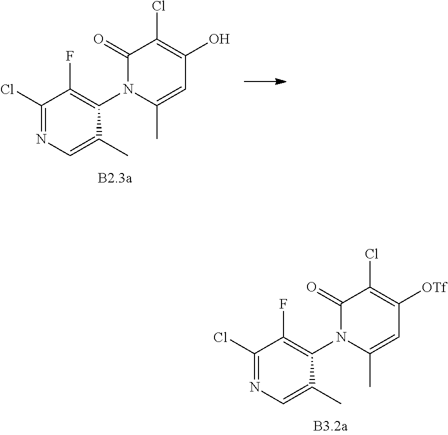

2′-bromo-3-chloro-3′-fluoro-5′,6-dimethyl-2-oxo-2H-[1,4′-bipyridin]-4-yl trifluoromethanesulfonate (B3.3a) B3.2a was prepared analogously to B3.1, using B2.2a.

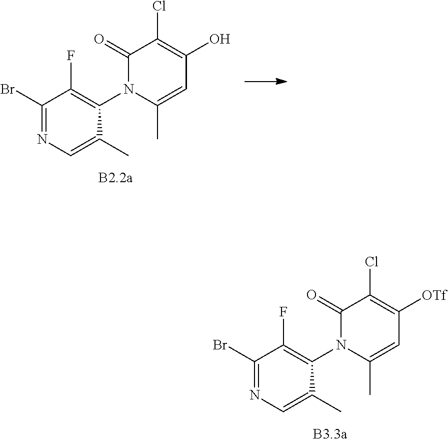

### Preparation 2′-bromo-3-chloro-4-((4-methoxybenzyl)oxy)-5′,6-dimethyl-2H-[1,4′-bipyridin]-2-one (B4.1)

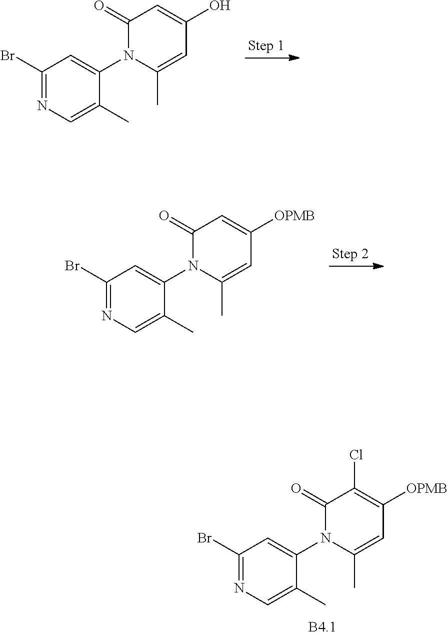

Step 1. A reactor was charged with 2′-bromo-4-hydroxy-5′,6-dimethyl-2H-[1,4′-bipyridin]-2-one (220 g, 745 mmol) and DMF (1.5 L), followed by K₂CO₃ (103 g, 745 mmol). 4-methoxybeznyl chloride (116 g, 745 mmol) was added dropwise and the mixture was heated to 60° C. for 1 hr. The mixture was cooled to ambient temperature and poured into water (7.7 L) and filtered. The solids were dried under vacuum to yield 2′-bromo-4-((4-methoxybenzyl)oxy)-5′,6-dimethyl-2H-[1,4′-bipyridin]-2-one. ¹H NMR (400 MHz, DMSO-d₆) δ 7.60-7.55 (m, 1H), 7.46 (d, J=2.0 Hz, 1H), 7.43-7.34 (m, 3H), 6.99 (d, J=8.8 Hz, 2H), 6.08 (d, J=1.8 Hz, 1H), 5.91 (d, J=2.4 Hz, 1H), 5.08-4.99 (m, 2H), 3.78 (s, 3H), 1.94 (s, 3H), 1.82 (s, 3H)

Step 2. A reactor was charged with 2′-bromo-4-((4-methoxybenzyl)oxy)-5′,6-dimethyl-2H-[1,4′-bipyridin]-2-one (230 g, 553 mmol), and MeCN (1.6 L), followed by NCS (111 g, 830 mmol). The mixture as heated to 60° C. for 2 hr. The mixture was cooled to ambient temperature and poured into water (8.1 L). The solids were dried under vacuum to yield 2′-bromo-3-chloro-4-((4-methoxybenzyl)oxy)-5′,6-dimethyl-2H-[1,4′-bipyridin]-2-one (B4.1). ¹H NMR (400 MHz, DMSO-d₆) δ 7.61 (dd, J=8.4, 2.0 Hz, 1H), 7.55 (d, J=1.8 Hz, 1H), 7.44 (d, J=8.6 Hz, 2H), 7.39 (d, J=8.2 Hz, 1H), 7.00 (d, J=8.6 Hz, 2H), 6.71 (s, 1H), 5.25 (s, 2H), 3.78 (s, 3H), 1.92 (s, 6H)

### Preparation of 2′-bromo-3-chloro-4-hydroxy-5′,6-dimethyl-2H-[1,4′-bipyridin]-2-one (B5.1, B5.1a, B5.1b)

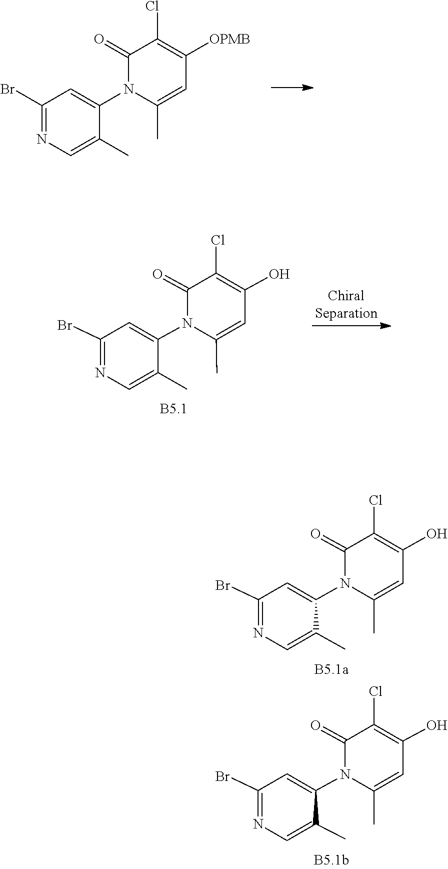

A reactor was charged with 2′-bromo-3-chloro-4-((4-methoxybenzyl)oxy)-5′,6-dimethyl-2H-[1,4′-bipyridin]-2-one (B4.1) (150 g, 333 mol) and DCM (750 mL), followed by TFA (50 mL, 76 g, 667 mmol). The mixture was stirred at ambient temperature for 30 min. The mixture was concentrated and the residue was subjected to flash column chromatography (SiO₂, petroleum ether-EtOAc) to yield the title compound (B5.1).

Individual atropisomers B5.1a and B5.1b were obtained by preparative SFC (DAICEL CHIRALPAK IC 250×50 mm, 10 μm; CO₂-0.1% NH₄OH in MeOH)

### Preparation of methyl (1S,2S)-2-((S)-2′,3-dichloro-5′,6-dimethyl-2-oxo-2H-[1,4′-bipyridin]-4-yl)cyclopropane-1-carboxylate (B6.1)

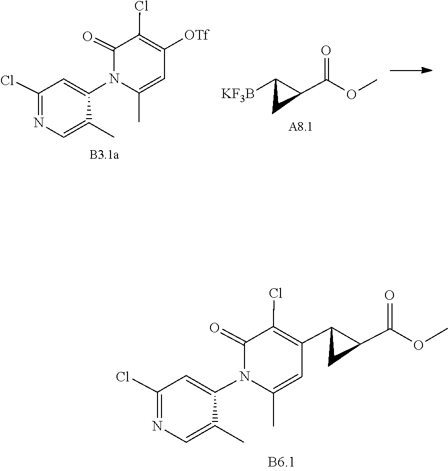

A flask was charged with 2′,3-dichloro-5′,6-dimethyl-2-oxo-2H-[1,4′-bipyridin]-4-yl trifluoromethanesulfonate (B3.1a) (1.25 g, 3.00 mmol), methyl (1S,2S)-2-(trifluoro-λ⁴-boraneyl)cyclopropane-1-carboxylate potassium salt (A8.1)(1.23 g, 6.00 mmol), Pd(dppf)Cl₂-DCM (817 mg, 0.300 mmol), and Cs₂CO₃ (2.93 g, 9.00 mmol), followed by PhMe (20 mL) and water (5 mL). The mixture was sparged with argon, and heated to 90° C. for 45 min. The mixture was cooled to ambient temperature, filtered, and concentrated. The residue was subjected to flash column chromatography (SiO₂, hexane-EtOAc) to afford (1S,2S)-2-((S)-2′,3-dichloro-5′,6-dimethyl-2-oxo-2H-[1,4′-bipyridin]-4-yl)cyclopropane-1-carboxylate (B6.1).

Preparation of methyl (1S,2S)-2-(2′,3-dichloro-3′-fluoro-5′,6-dimethyl-2-oxo-2H-[1,4′-bipyridin]-4-yl)cyclopropane-1-carboxylate (B6.2). B6.2 was prepared analogously to B6.1, using B3.2a instead of B3.1a.

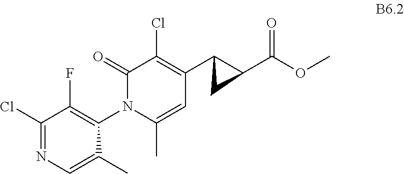

### Preparation of 3-chloro-4-hydroxy-2′-(2-(2-hydroxypropan-2-yl)pyrimidin-4-yl)-5′,6-dimethyl-2H-[1,4′-bipyridin]-2-one (B7.1)

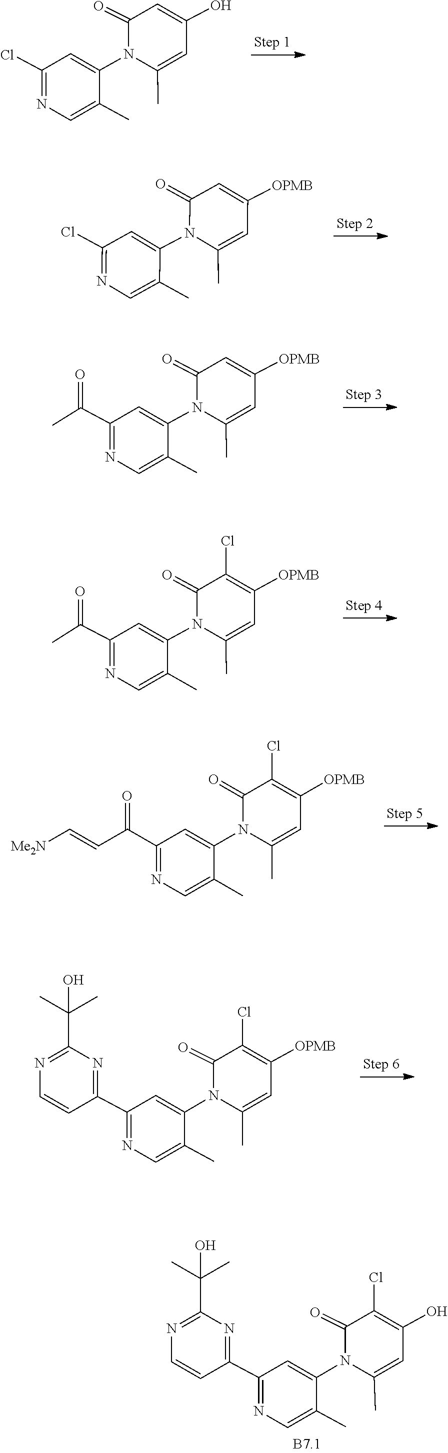

Step 1. A flask was charged with 2′-chloro-4-hydroxy-5′,6-dimethyl-2H-[1,4′-bipyridin]-2-one-2′ (B1.1) (116 g, 465 mmol), 18-crown-6 (2.46 g, 9.29 mmol), K₂CO₃ (193 g, 1.40 mol), and DMF (1.4 L), followed by PMB-Cl (72.8 g, 465 mmol). The mixture was heated to 50° C. for 12 hrs, after which it was cooled to room temperature. Water (2 L) was added, and the mixture was extracted with EtOAc (2×1 L). The combined organic layers were washed with NaCl (sat. aq., 500 mL×2), dried over Na₂SO₄, filtered, and concentrated to give 2′-chloro-4-((4-methoxybenzyl)oxy)-5′,6-dimethyl-2H-[1,4′-bipyridin]-2-one.

¹H NMR (DMSO-d₆ 400 MHz): δ 8.49 (s, 1H) 7.59 (s, 1H) 7.39 (d, J=8.6 Hz, 2H) 6.97 (d, J=8.6 Hz, 2H) 6.11 (d, J=1.8 Hz, 1H) 5.93 (d, J=2.4 Hz, 1H) 5.03 (s, 2H) 3.77 (s, 3H) 1.98 (s, 3H) 1.85 (s, 3H)

Step 2. A flask was charged with 2′-chloro-4-((4-methoxybenzyl)oxy)-5′,6-dimethyl-2H-[1,4′-bipyridin]-2-one (85 g, 216 mmol) and tributyl(1-ethoxyvinyl)stannane (114 g, 318 mmol). The flask was evacuated and backfilled with N₂ three times, and was then charged with Pd(dppf)Cl₂ (20.4 g, 27.8 mmol) and dioxane (1.1 L). The mixture was heated to 110° C. for 12 hrs, after which it was cooled to ambient temperature. HCL (2M, 350 mL) was added and the mixture was stirred for 30 min. The mixture was then adjusted to pH=7 with NaHCO₃. KF (500 g) was added and the mixture stirred for 1 hr. Water (500 mL) was added, and the mixture extracted with EtOAc (2×1 L), The combined organic layers were washed with NaCl (sat. aq., 500 mL), dried over Na₂SO₄, filtered, and concentrated. The resulting solids were triturated with TBME to yield 2′-acetyl-4-((4-methoxybenzyl)oxy)-5′,6-dimethyl-2H-[1,4′-bipyridin]-2-one. ¹H NMR (DMSO-d₆ 400 MHz): δ 8.80 (s, 1H) 7.78 (s, 1H) 7.40 (br d, J=8.2 Hz, 2H) 6.98 (br d, J=8.4 Hz, 2H) 6.12 (s, 1H) 5.94 (s, 1H) 5.04 (s, 2H) 3.78 (s, 3H) 2.67 (s, 3H) 2.10 (s, 3H) 1.81 (s, 3H)

Step 3. A flask was charged with 2′-acetyl-4-((4-methoxybenzyl)oxy)-5′,6-dimethyl-2H-[1,4′-bipyridin]-2-one (59 g, 140 mmol), Cl₂HCO₂H (1.85 g, 14.0 mmol), and iPrOH (530 mL). The mixture was heated to 60° C. and NCS (20.8 g, 156 mmol) was added slowly in three portions. The mixture was stirred a further 1 hr, cooled to room temperature, and filtered, and washed with iPrOH. The filtrate was concentrated to afford 2′-acetyl-3-chloro-4-((4-methoxybenzyl)oxy)-5′,6-dimethyl-2H-[1,4′-bipyridin]-2-one. ¹H NMR (DMSO-d₆ 400 MHz): δ 8.83 (s, 1H) 7.88 (s, 1H) 7.45 (d, J=8.6 Hz, 2H) 7.01 (d, J=8.6 Hz, 2H) 6.76 (s, 1H) 5.27 (s, 2H) 3.79 (s, 3H) 2.67 (s, 3H) 2.09 (s, 3H) 1.92 (s, 3H)

Step 4. A flask was charged with 2′-acetyl-3-chloro-4-((4-methoxybenzyl)oxy)-5′,6-dimethyl-2H-[1,4′-bipyridin]-2-one (46 g, 100 mmol), DMF-DMA (67 mL, 501 mmol) and DMF (460 mL). The mixture was heated to 70° C. for 12 hrs and cooled to room temperature. The crude solution was used directly in the next step. ES/MS: m/z 468.1 [M+H]⁺.

Step 5. To the solution above was added 2-hydroxy-2-methylpropanimidamide hydrochloride (69.5 g, 501 mmol), K₂CO₃ (97 g, 702 mmol), and DMF (460 mL). The mixture was heated to 75° C. for 48 hrs and then cooled to ambient temperature. Water (1.2 L) was added the mixture was extracted with EtOAc (2×1 L). The combined organic layers were washed with NaCl (sat. aq., 500 mL×2), dried over Na₂SO₄, filtered, and concentrated to give 3-chloro-2′-(2-(2-hydroxypropan-2-yl)pyrimidin-4-yl)-4-((4-methoxybenzyl)oxy)-5′,6-dimethyl-2H-[1,4′-bipyridin]-2-one. ¹H NMR (DMSO-d₆400 MHz): δ 8.97 (d, J=5.2 Hz, 1H) 8.85 (s, 1H) 8.67 (s, 1H) 8.23 (d, J=5.2 Hz, 1H) 7.45 (d, J=8.6 Hz, 2H) 7.01 (d, J=8.6 Hz, 2H) 6.80 (s, 1H) 5.27 (d, J=16.6 Hz, 3H) 3.78 (s, 3H) 2.10 (s, 3H) 1.98 (s, 3H) 1.53 (d, J=4.8 Hz, 6H)

Step 6. A vial was charged with 3-chloro-2′-(2-(2-hydroxypropan-2-yl)pyrimidin-4-yl)-4-((4-methoxybenzyl)oxy)-5′,6-dimethyl-2H-[1,4′-bipyridin]-2-one (37 g, 65.7 mmol), TFA (80 mL) and DCM (350 mL). The mixture was stirred at ambient temperature for 2 hrs. The mixture was concentrated, diluted with water (150 mL), and extracted with 10:1 DCM/MeOH (2×500 mL). The combined organic layers were washed with NaCl (sat. aq., 500 mL×2), dried over Na₂SO₄, filtered, and concentrated. The residue was triturated with TBME and dried under vacuum to give 3-chloro-4-hydroxy-2′-(2-(2-hydroxypropan-2-yl)pyrimidin-4-yl)-5′,6-dimethyl-2H-[1,4′-bipyridin]-2-one (B7.1). ¹H NMR (DMSO-d₆ 400 MHz): δ 11.63 (br s, 1H) 8.96 (d, J=5.2 Hz, 1H) 8.84 (s, 1H) 8.65 (s, 1H) 8.24 (d, J=5.2 Hz, 1H) 6.21 (s, 1H) 2.09 (s, 3H) 1.88 (s, 3H) 1.53 (d, J=4.4 Hz, 6H)

3-chloro-3′-fluoro-4-hydroxy-2′-(2-(2-hydroxypropan-2-yl)pyrimidin-4-yl)-5′,6-dimethyl-2H-[1,4′-bipyridin]-2-one (B7.2). B7.1 was prepared using step 2, step 4, and step 5 in the preparation of B7.1 starting with B2.2 instead of B1.1. ES/MS m/z: 405.1 [M+H]+

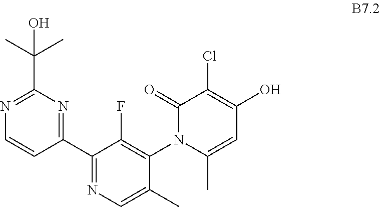 [SMILES: Cc1cnc(-c2ccnc(C(C)(C)O)n2)c(F)c1-n1c(C)cc(O)c(Cl)c1=O]

### Chiral Separation of B7.1 into B7.1a B7.1b

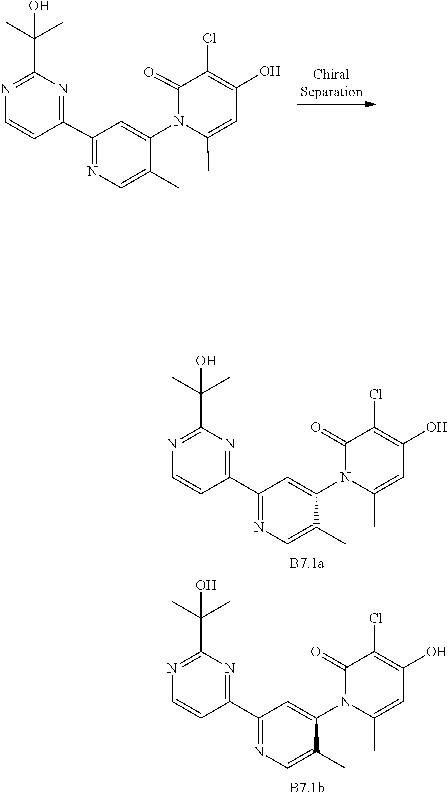

The isomers were separated by SFC (50% MeOH in CO₂ ChiralPak IG 4.6×100 mm, 3.0 mL/min).

### Preparation of 3-chloro-2′-(2-(2-hydroxypropan-2-yl)pyrimidin-4-yl)-5′,6-dimethyl-2-oxo-2H-[1,4′-bipyridin]-4-yl trifluoromethanesulfonate (B8.1, B8.1a)

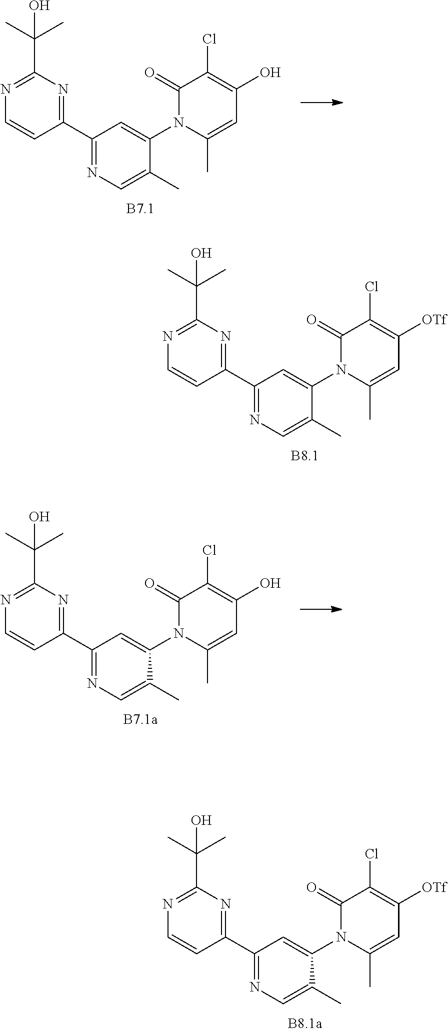

A vial was charged with 3-chloro-4-hydroxy-2′-(2-(2-hydroxypropan-2-yl)pyrimidin-4-yl)-5′,6-dimethyl-2H-[1,4′-bipyridin]-2-one (800 mg, 2.07 mmol), Et₃N (0.87 mL, 6.2 mmol), and DCM (10 mL). The mixture was cooled to 0° C. and Tf₂O (0.52 mL, 3.10 mmol) was added dropwise. The mixture was stirred for 1 hr, before water (10 mL) was added. The mixture was extracted with DCM (3×10 mL). The combined organics were dried over Na₂SO₄, filtered, and concentrated. The residue was subjected to flash column chromatography (SiO₂, hexanes-EtOAc) to give 3-chloro-2′-(2-(2-hydroxypropan-2-yl)pyrimidin-4-yl)-5′,6-dimethyl-2-oxo-2H-[1,4′-bipyridin]-4-yl trifluoromethanesulfonate (B8.1). ES/MS m/z: 519.1 [M+H]+

This procedure could also be conducted on atropisomerically enriched material 7.1a to give atropisomerically enriched material 8.1a.

3-chloro-3′-fluoro-2′-(2-(2-hydroxypropan-2-yl)pyrimidin-4-yl)-5′,6-dimethyl-2-oxo-2H-[1,4′-bipyridin]-4-yl trifluoromethanesulfonate (B8.2) B8.2 was prepared analogously to B8.1, using N-Phenylbis(trifluoromethane)sulfonimide instead of Tf₂O, DMF instead of DCM, and B7.2 instead of B7.1. ES/MS m/z: 537.0 [M+H]+

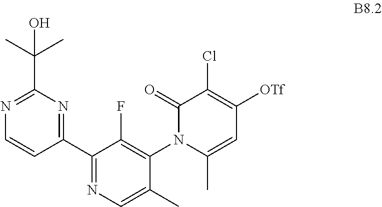 [SMILES: Cc1cnc(-c2ccnc(C(C)(C)O)n2)c(F)c1-n1c(C)cc(OS(=O)(=O)C(F)(F)F)c(Cl)c1=O]

### 5-chloro-3-(2-chloro-3-fluoro-5-methylpyridin-4-yl)-2,6-dimethylpyrimidin-4(3H)-one (B9.1, B9.1a, and B9.1b)

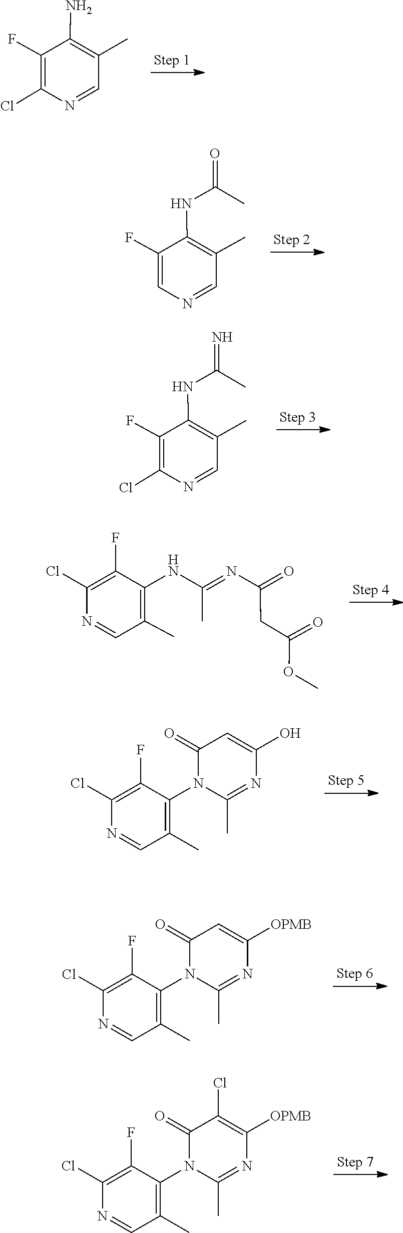

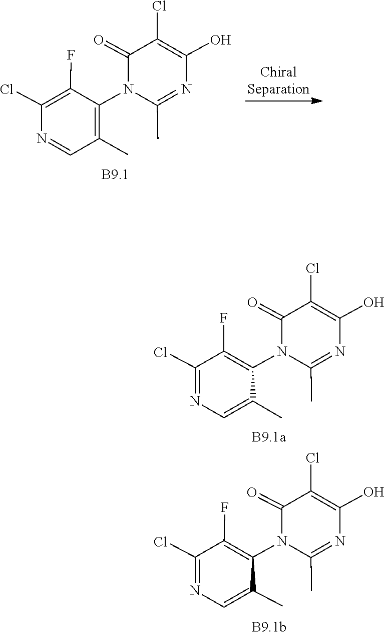

Step 1. A solution of 2-chloro-3-fluoro-5-methylpyridin-4-amine (5.0 g, 31.1 mmol, 1.0 equiv.) and DMAP (38.0 mg, 0.3 mmol, 0.01 equiv.) in acetic anhydride (80 mL) was stirred for 3 h at 140° C. under air atmosphere. The resulting mixture was concentrated under reduced pressure. To the above mixture was added NaOH (3.7 g, 93.4 mmol, 3 equiv.), MeOH (40 mL) and H₂O (10 mL) in one portion at room temperature. The resulting mixture was stirred for additional 10 min at room temperature. The resulting mixture was extracted with EtOAc (3×150 mL). The combined organic layers were dried over anhydrous Na₂SO₄. After filtration, the filtrate was concentrated under reduced pressure. This resulted in N-(2-chloro-3-fluoro-5-methylpyridin-4-yl) acetamide. LCMS (ESI, m/z): [M+H]⁺=203.05

Step 2. A solution of N-(2-chloro-3-fluoro-5-methylpyridin-4-yl) acetamide (5.6 g, 27.6 mmol, 1.0 equiv.) and PCl₅ (5.8 g, 27.6 mmol, 1.0 equiv.) in Toluene (100 mL) was stirred overnight at 120° C. under nitrogen atmosphere. The resulting mixture was concentrated under reduced pressure. Then the mixture of crude product in THF (80 mL) was stirred for 30 min at −78° C. under ammonia atmosphere. The resulting mixture was partitioned between EtOAc (3×150 mL) and water (150 mL). The combined organic layers were washed with brine (1×150 mL), dried over anhydrous Na₂SO₄. After filtration, the filtrate was concentrated under reduced pressure. This resulted in N-(2-chloro-3-fluoro-5-methylpyridin-4-yl) ethanimidamide. LCMS (ESI): [M+H]⁺=202.15

Step 3. To a stirred solution of N-(2-chloro-3-fluoro-5-methylpyridin-4-yl) ethanimidamide (5.3 g, 26.3 mmol, 1 equiv.) and NMM (4.0 g, 39.4 mmol, 1.5 equiv.) in DCM (100 mL) was added methyl 3-chloro-3-oxopropanoate (5.4 g, 39.4 mmol, 1.5 equiv.) in portions at 0° C. under nitrogen atmosphere. The resulting mixture was stirred for 2 h at room temperature under nitrogen atmosphere. The reaction was quenched with NaHCO₃ at room temperature. The resulting mixture was extracted with EtOAc (3×150 mL). The combined organic layers were washed with water (3×150 mL), dried over anhydrous Na₂SO₄. After filtration, the filtrate was concentrated under reduced pressure. The residue was purified by silica gel column chromatography, eluted with PE/EA (2:1) to afford methyl 2-{[(1E)-1-[(2-chloro-3-fluoro-5-methylpyridin-4-yl) amino] ethylidene] carbamoyl} acetate. LCMS (ESI): [M+H]⁺=302.20

Step 4. To a stirred solution of methyl 2-{[(TE)-1-[(2-chloro-3-fluoro-5-methylpyridin-4-yl) amino] ethylidene] carbamoyl} acetate (4.5 g, 14.9 mmol, 1.0 equiv.) in 1,4-dioxane (80 mL) was added DBU (6.8 g, 44.7 mmol, 3.0 equiv.) in portions at 0° C. under nitrogen atmosphere. The resulting mixture was stirred overnight at 60° C. under nitrogen atmosphere. The resulting mixture was concentrated under reduced pressure. The residue was purified by silica gel column chromatography, eluted with CH₂Cl₂/MeOH (10:1) to afford 3-(2-chloro-3-fluoro-5-methylpyridin-4-yl)-6-hydroxy-2-methylpyrimidin-4-one. LCMS (ESI): [M+H]⁺=270.0

Step 5. To a stirred solution of 3-(2-chloro-3-fluoro-5-methylpyridin-4-yl)-6-hydroxy-2-methylpyrimidin-4-one (2.7 g, 10.0 mmol, 1 equiv.) and K₂CO₃ (4.1 g, 30.0 mmol, 3.0 equiv.) in DMF (40 mL) was added 4-methoxybenzyl chloride (1.6 g, 10.0 mmol, 1.0 equiv.) dropwise at 0° C. under nitrogen atmosphere. The resulting mixture was stirred for 2 h at 60° C. under nitrogen atmosphere. The resulting mixture was extracted with EtOAc (3×80 mL). The combined organic layers were washed with water (3×80 mL), dried over anhydrous Na₂SO₄. After filtration, the filtrate was concentrated under reduced pressure. The residue was purified by silica gel column chromatography, eluted with PE/EA (2:1) to afford 3-(2-chloro-3-fluoro-5-methylpyridin-4-yl)-6-[(4-methoxyphenyl) methoxy]-2-methylpyrimidin-4-one. LCMS (ESI): [M+H]⁺=390.10

Step 6. To a stirred solution of 3-(2-chloro-3-fluoro-5-methylpyridin-4-yl)-6-[(4-methoxyphenyl) methoxy]-2-methylpyrimidin-4-one (2.0 g, 5.1 mmol, 1.0 equiv.) and NCS (822.1 mg, 6.1 mmol, 1.2 equiv.) in i-PrOH (20 mL) was added 2,2-dichloroacetic acid (66.2 mg, 0.5 mmol, 0.1 equiv.) in portions at room temperature under air atmosphere. The resulting mixture was stirred for 4 h at 60° C. The resulting mixture was extracted with EtOAc (3×50 mL). The combined organic layers were washed with water (3×50 mL), dried over anhydrous Na₂SO₄. After filtration, the filtrate was concentrated under reduced pressure. The residue was purified by silica gel column chromatography, eluted with PE/EA (2:1) to afford 5-chloro-3-(2-chloro-3-fluoro-5-methylpyridin-4-yl)-6-[(4-methoxyphenyl) methoxy]-2-methylpyrimidin-4-one. LCMS (ESI): [M+H]⁺=424.05

Step 7. A solution of 5-chloro-3-(2-chloro-3-fluoro-5-methylpyridin-4-yl)-6-[(4-methoxyphenyl) methoxy]-2-methylpyrimidin-4-one (1.5 g, 3.5 mmol, 1 equiv.) and HCl (gas) in 1,4-dioxane (0.6 g, 17.7 mmol, 5.0 equiv.) in DCM (20 mL) was stirred for 30 min at room temperature under air atmosphere. The reaction was monitored by LCMS. The precipitated solids were collected by filtration and washed with DCM (3×10 mL). The resulting mixture was concentrated under reduced pressure. to afford 5-chloro-3-(2-chloro-3-fluoro-5-methylpyridin-4-yl)-6-hydroxy-2-methylpyrimidin-4-one (B9.1). LCMS (ESI): [M+H]⁺=304.05 ¹H NMR (400 MHz, Methanol-d₄): δ 8.34 (s, 1H), 2.20 (s, 3H), 1.88-2.18 (m, 3H).

B9.1 was separated into stereoisomers B9.1a and B9.1b by SFC (CHIRAL ART Cellulose-SC, 3×25 cm, 5 μM; 70% MeOH with 0.1% NH₃ in CO₂, 100 mL/min, 35° C.

### (R)-5-chloro-1-(2-chloro-3-fluoro-5-methylpyridin-4-yl)-2-methyl-6-oxo-1,6-dihydropyrimidin-4-yl trifluoromethanesulfonate (B10.1)

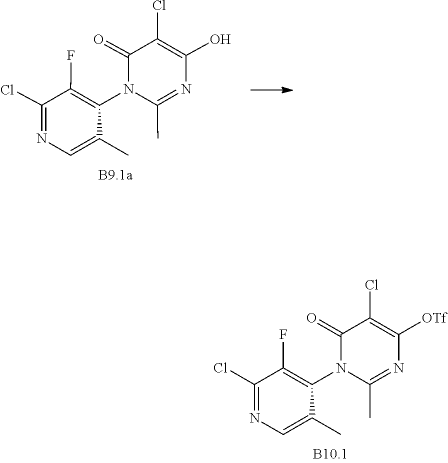

A flask was charged with B9.1a (545 mg, 1.79 mmol), DMF (7 mL), and Et₃N (0.45 g, 4.48 mmol). N-(5-chloropyridin-2-yl)-1,1,1-trifluoro-N-((trifluoromethyl)sulfonyl)methanesulfonamide (1.76 g, 4.48 mmol) was added. The mixture stirred for 2 hrs. H₂O (30 mL) was added, the mixture was filtered and the solids washed with H₂O (5 mL). The resulting solids were dried under vacuum and subject to flash column chromatography (SiO₂, hexane-EtOAc) to give (R)-5-chloro-1-(2-chloro-3-fluoro-5-methylpyridin-4-yl)-2-methyl-6-oxo-1,6-dihydropyrimidin-4-yl trifluoromethanesulfonate.

The following intermediates were purchased from commercial vendors:

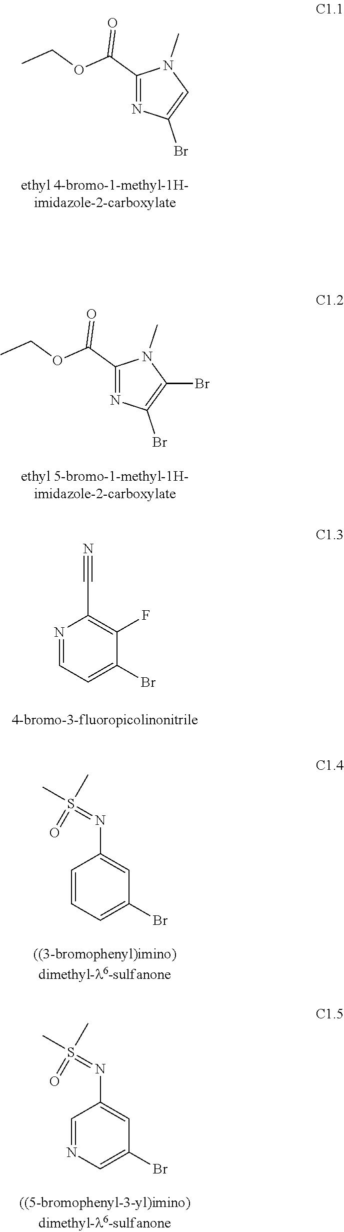

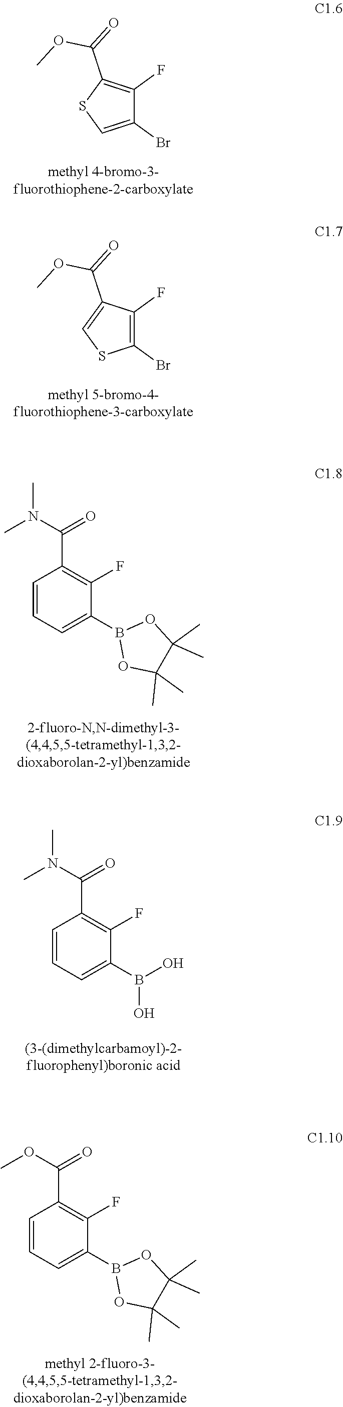

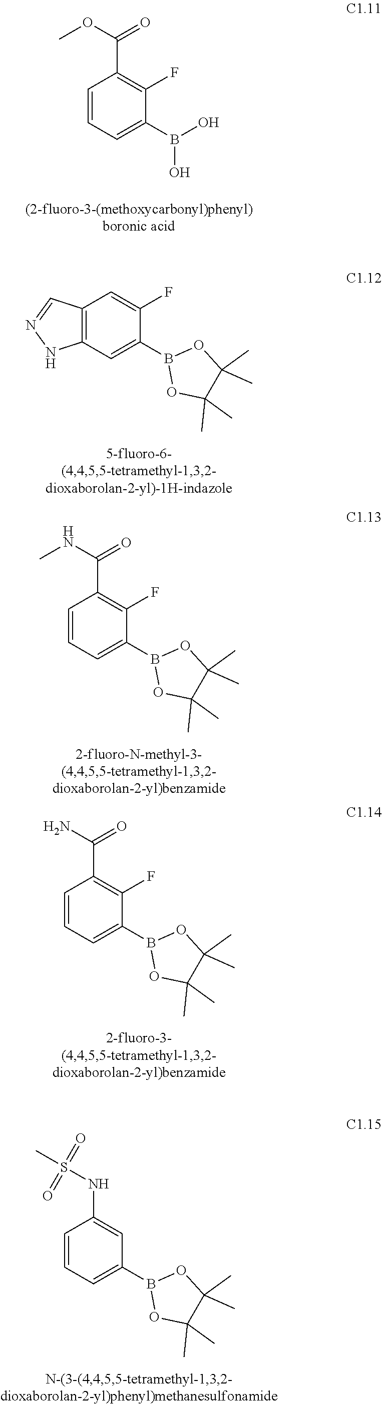

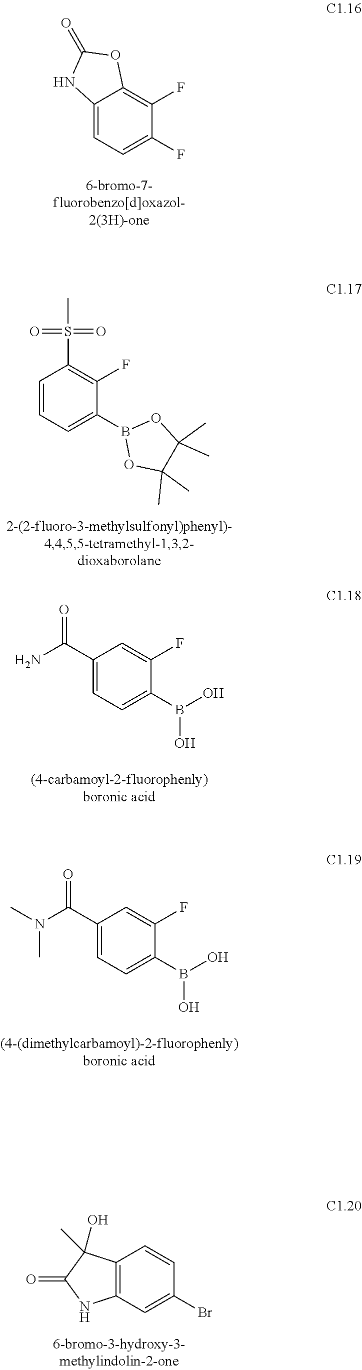

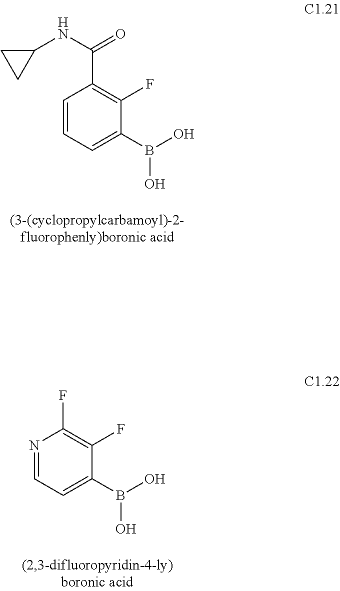 [SMILES: O=C(NC1CC1)c1cccc(B(O)O)c1F ; OB(O)c1ccnc(F)c1F]

### Preparation of 6-bromo-5-fluoro-1-tetrahydropyran-2-yl-indazole-4-carboxylate (C2.1)

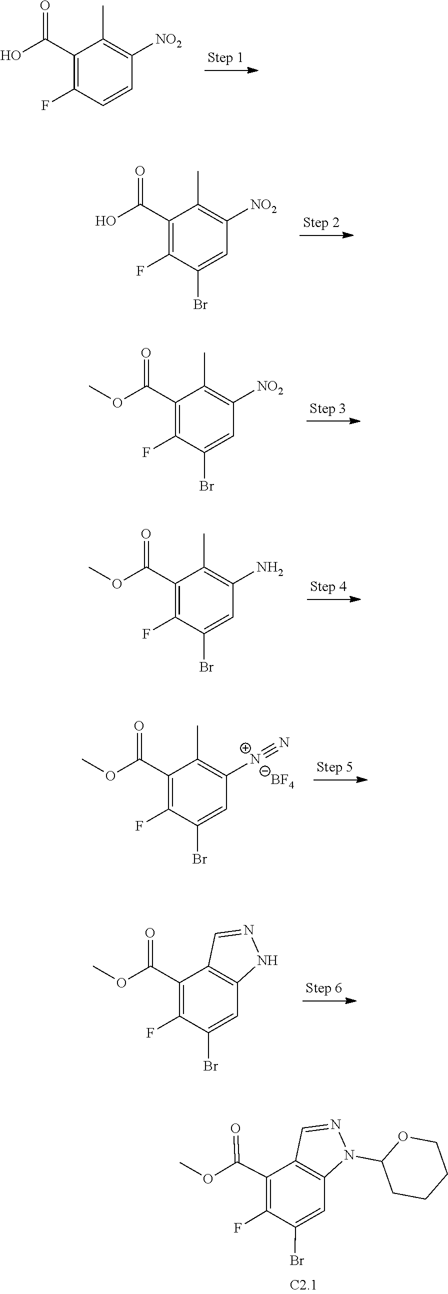

Step 1: To a flask of 6-fluoro-2-methyl-3-nitro-benzoic acid (2.50 g, 12.6 mmol) in sulfuric acid (10 mL) was added 1,3-dibromo-5,5-dimethylhydantoin (1.97 g, 6.90 mmol) portion-wise, keeping the reaction from boiling. The reaction was stirred at room temperature for 16 hrs and then poured into ice water, giving a precipitate which was filtered off and washed with H₂O. After drying under high vacuum, 3-bromo-2-fluoro-6-methyl-5-nitro-benzoic acid was isolated.

Step 2: To a flask, 3-bromo-2-fluoro-6-methyl-5-nitro-benzoic acid (1.5 g, 5.40 mmol) and Na₂CO₃ (2.29 g, 21.6 mmol) were charged and placed under a flow of N₂. The solids were dissolved in DMF (12 mL) followed by the addition of methyl iodide (3.06 g, 21.6 mmol). The reaction was stirred overnight at 60° C. After cooling to room temperature, the reaction was diluted with H₂O and EtOAc and the layers were separated. The aqueous was extracted with EtOAc (3×), and the organic extracts were combined, dried with MgSO₄, filtered, and concentrated to yield methyl 3-bromo-2-fluoro-6-methyl-5-nitro-benzoate, which was used without further purification.

Step 3: To a flask, methyl 3-bromo-2-fluoro-6-methyl-5-nitro-benzoate from Step 2 was charged followed by NH₄Cl (1.45 g, 27.0 mmol) and Fe powder (0.906 g, 16.2 mmol). The solid was dissolve in EtOH:H₂O (1:1, 10 mL) and heated at 95° C. After cooling to room temperature, the reaction was filtered through celite and diluted with H₂O and EtOAc and the layers were separated. The aqueous was extracted with EtOAc (3×), and the organic extracts were combined, dried with MgSO₄, filtered, and concentrated to yield methyl 5-amino-3-bromo-2-fluoro-6-methyl-benzoate, which was used without further purification. ES/MS: m/z 263.2 [M+H]+.

Step 4: methyl 5-amino-3-bromo-2-fluoro-6-methyl-benzoate from Step 3 and NH₄BF₄ (1.09 g, 10.4 mmol) were charged to a flask. The solids were dissolved in AcOH (15 mL) and H₂O (7 mL) and the reaction was cooled to 0° C. Concentrated HCl (2.61 mL, 31.3 mmol) was added and stirred for 5 mins. To the flask, NaNO₂ (0.396 g, 5.73 mmol) was added and stirred at 0° C. for 5 mins, then 1 hr at 25° C. The reaction was concentrated and then azeotroped with PhMe to dryness to afford 5-bromo-4-fluoro-3-methoxycarbonyl-2-methyl-benzenediazonium; tetrafluoroborate, which was used immediately without further purification. ES/MS: m/z 258.1 [M+H]+.

Step 5: 5-bromo-4-fluoro-3-methoxycarbonyl-2-methyl-benzenediazonium; tetrafluoroborate from Step 4 was charged to a flask along with KOAc (5.12 g, 52.1 mmol). The solids were diluted with EtOAc (25 mL) and stirred at room temperature for 16 hr. After completion, the reaction was diluted with EtOAc and washed with brine. The aqueous was extracted with EtOAc (3×), and the organic extracts were combined, dried with MgSO₄, filtered, and concentrated. The crude material was subjected to silica gel flash chromatography (hexanes to EtOAc) to yield methyl 6-bromo-5-fluoro-1H-indazole-4-carboxylate. ES/MS: m/z 274.7 [M+H]+.

Step 6: To a flask methyl 6-bromo-5-fluoro-1H-indazole-4-carboxylate (0.263 g, 0.963 mmol) and TsOH·H₂O (0.018 g, 0.963 mmol) were charged and dissolved in DCM (10 mL). To the flask, 3,4-Dihydro-2H-pyran (0.243 g, 2.89 mmol) was added and the reaction was stirred at room temperature for 16 hr. The reaction was quenched by the addition of sat. aq. NaHCO₃. The aqueous was extracted with EtOAc (3×), and the organic extracts were combined, dried with MgSO₄, filtered, and concentrated. The crude material was subjected to silica gel flash chromatography (hexanes to EtOAc) to yield methyl 6-bromo-5-fluoro-1-tetrahydropyran-2-yl-indazole-4-carboxylate (C2.1).

### Preparation of 5-bromo-4-fluoro-3,3-dimethylindolin-2-one (C3.1)

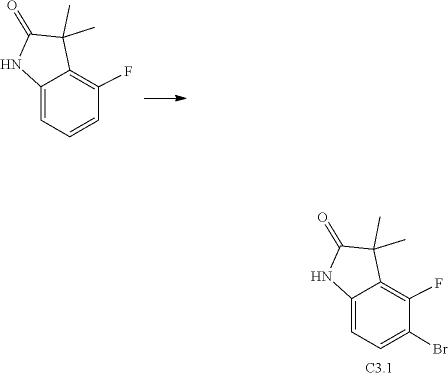

To a flask, 4-fluoro-3,3-dimethyl-indolin-2-one (1.00 g, 5.58 mmol) was charged and dissolved in DCM (50 mL) and AcOH (2 mL). To the flask, Br₂ (0.387 mL, 7.53 mmol) was added dropwise and the reaction was stirred for 16 hr at room temperature. The reaction was poured into a solution of sat. aq. Na₂S₂O₃. The aqueous was extracted with EtOAc (3×), and the organic extracts were combined, dried with MgSO₄, filtered, and concentrated. The crude material was subjected to silica gel flash chromatography (hexanes to EtOAc) to yield 5-bromo-4-fluoro-3,3-dimethyl-indolin-2-one (C3.1). ES/MS: m/z 259.8 [M+H]+.

### Preparation of tert-butyl (3-bromo-2-fluorophenyl)carbamate (C4.1)

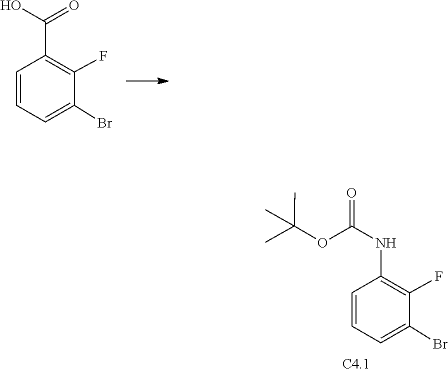 [SMILES: O=C(O)c1cccc(Br)c1F ; CC(C)(C)OC(=O)Nc1cccc(Br)c1F]

A flask was charged with 3-bromo-2-fluoro-benzoic acid (6 g, 27 mmol) and triethylamine (4.96 mL, 36 mmol), and tert-butanol (28 mL), followed by diphenylphosphoryl azide (6.49 mL, 30.1 mmol). The mixture was heated to 80° C. for 16 hrs, after which it was cooled to room temperature. The mixture was diluted with ethyl acetate and washed sequentially with saturated ammonium chloride, saturated sodium bicarbonate, and saturated sodium chloride. The organic layer was dried over anhydrous sodium sulfate, filtered, and concentrated. The residue was subjected to flash chromatography (SiO₂, hexane-EtOAc) to yield tert-butyl N-(3-bromo-2-fluoro-phenyl)carbamate (C4.1).

### Preparation of 3-hydroxy-6-iodo-3-isopropyl-4-(trifluoromethyl)-1-(2-trimethylsilylethoxymethyl)indolin-2-one (C5.1)

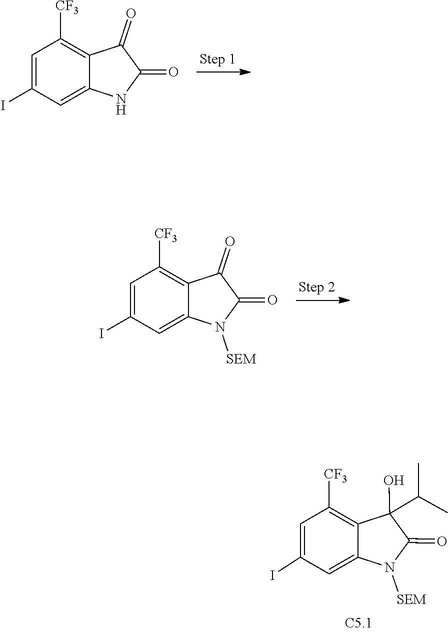 [SMILES: O=C1Nc2cc(I)cc(C(F)(F)F)c2C1=O ; C[Si](C)(C)CCOCN1C(=O)C(=O)c2c1cc(I)cc2C(F)(F)F ; CC(C)C1(O)C(=O)N(COCC[Si](C)(C)C)c2cc(I)cc(C(F)(F)F)c21]

Step 1. A flask was charged with 6-iodo-4-(trifluoromethyl)indoline-2,3-dione (380 mg, 1.11 mmol) and DMF, and it was cooled to 0° C. under dry nitrogen. To this, sodium hydride (60% in mineral oil, 53.5 mg, 1.34 mmol) was added, and the reaction was stirred for 30 minutes. To this, 2-(chloromethoxy)ethyl-trimethyl-silane (0.24 mL, 1.3 mmol) was added, and the reaction was allowed to warm to ambient temperature and stir for 16 hours. It was quenched by the addition of saturated aqueous ammonium chloride, extracted once with ethyl acetate, and the extract was washed with water and saturated sodium chloride. It was dried over anhydrous sodium sulfate, filtered, and concentrated. It was purified by flash chromatography (SiO₂, hexane-EtOAc) to yield 6-iodo-4-(trifluoromethyl)-1-(2-trimethylsilylethoxymethyl)indoline-2,3-dione.

Step 2. A flame-dried flask was charged with 6-iodo-4-(trifluoromethyl)-1-(2-trimethylsilylethoxymethyl)indoline-2,3-dione (150 mg, 0.32 mmol) and tetrahydrofuran (3 mL) under dry nitrogen, and it was cooled to −78° C. To this, isopropylmagnesium chloride (2.0M in THF, 0.19 mL, 0.38 mmol) was added dropwise, and the reaction was stirred at this temperature for 2 hours. It was quenched by the addition of saturated aqueous ammonium chloride and was allowed to warm to ambient temperature. The mixture was extracted with ethyl acetate, and the extract was washed with water and saturated sodium chloride. It was dried over anhydrous sodium sulfate, filtered, and concentrated. It was purified by flash chromatography (SiO₂, hexane-EtOAc) to yield the title compound (C5.1). ES/MS: m/z: 538.1 (M+Na)⁺.

6-bromo-5-fluoro-3-hydroxy-3-isopropyl-1-(2-trimethylsilylethoxymethyl)indolin-2-one (C5.2) C5.2 was prepared analogously to C5.1 using 6-bromo-5-fluoro-indoline-2,3-dione. ES/MS m/z: 440.1 (M+Na)⁺.

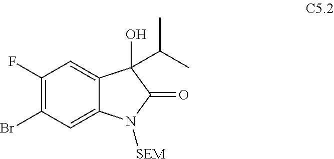 [SMILES: CC(C)C1(O)C(=O)N(COCC[Si](C)(C)C)c2cc(Br)c(F)cc21]

3-hydroxy-3-isopropyl-6-(4,4,5,5-tetramethyl-1,3,2-dioxaborolan-2-yl)-4-(trifluoromethyl)-1-((2-(trimethylsilyl)ethoxy)methyl)indolin-2-one (C5.3). C5.2 was prepared analogously to C5.1, step 2 using 6-bromo-5-fluoro-indoline-2,3-dione and methylmagnesium bromide.

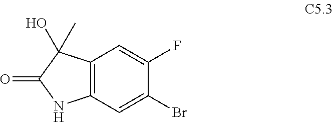

### 6-bromo-3,3-difluoro-1-(2-trimethylsilylethoxymethyl)indolin-2-one (C5.4)

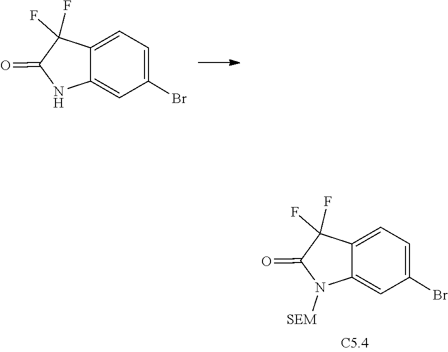 [SMILES: O=C1Nc2cc(Br)ccc2C1(F)F ; C[Si](C)(C)CCOCN1C(=O)C(F)(F)c2ccc(Br)cc21]

A vial was charged with 6-bromo-3,3-difluoro-indolin-2-one (351 mg, 1.42 mmol) and DMF (10.5 mL) followed by NaHMDS (1 M in THF, 1.7 mL, 1.7 mmol) and 2-(trimethylsilyl)ethoxymethyl chloride (0.3 mL, 1.7 mmol) at room temperature. After 1 hr, NH₄Cl (sat aq., 1 mL) was added. The reaction was extracted with ethyl acetate (3×2 mL), and the extracts were washed with NaCl (sat aq., 5 mL) and LiCl (1M, 5 mL) sequentially. It was dried over anhydrous sodium sulfate, filtered, and concentrated. It was purified by flash chromatography (SiO₂, hexane-EtOAc) to give 6-bromo-3,3-difluoro-1-(2-trimethylsilylethoxymethyl)indolin-2-one (C5.4).

### Preparation of N-(3-bromo-2-fluoro-phenyl)-2-hydroxy-2-methyl-propanamide (C6.1)

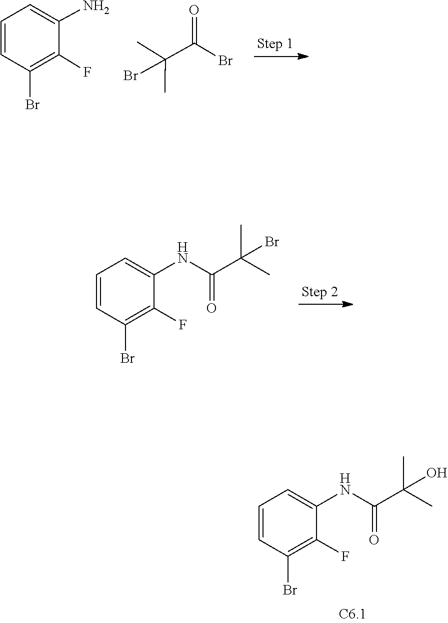

Step 1. A flask was charged with 3-bromo-2-fluoro-aniline (200 mg, 1.05 mmol), triethylamine (0.176 mL, 1.26 mmol), and dichloromethane (4 mL), then 2-bromo-2-methyl-propanoyl bromide (0.143 mL, 1.16 mmol) was added, and the reaction was allowed to stir at ambient temperature for 3 days. It was diluted with ethyl acetate and washed with 10% aqueous potassium bisulfate, saturated sodium bicarbonate, and saturated sodium chloride. It was dried over anhydrous sodium sulfate, filtered, and concentrated to yield 2-bromo-N-(3-bromo-2-fluoro-phenyl)-2-methyl-propanamide. ES/MS: m/z: 340.0 (M+H)⁺.

Step 2. A vial was charged with 2-bromo-N-(3-bromo-2-fluoro-phenyl)-2-methyl-propanamide (301 mg, 0.888 mmol) and acetonitrile (9 mL), and to this was added silver(I) oxide (412 mg, 1.78 mmol). The reaction was stirred at ambient temperature for 16 hours. It was filtered through celite, and the filter pad was washed with ethyl acetate. The combined filtrates were concentrated and purified by flash chromatography (SiO₂, hexane-EtOAc) to yield the title compound (C6.1). ES/MS: m/z: 276.4 (M+H)⁺.

N-(3-bromo-2-fluoro-phenyl)acetamide (C6.2). C6.2 was prepared from 3-bromo-2-fluoro-aniline using Step 1 in the synthesis of C6.1 and using acetic anhydride instead of 2-bromo-2-methyl-propanoyl bromide.

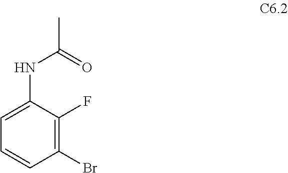

N-(3-bromo-2-fluorophenyl)-1-methylcyclopropane-1-carboxamide (C6.3). C6.3 was prepared from 3-bromo-2-fluoro-aniline using Step 1 in the synthesis of C6.1 and using 1-methylcyclopropane-1-carbonyl chloride instead of 2-bromo-2-methyl-propanoyl bromide.

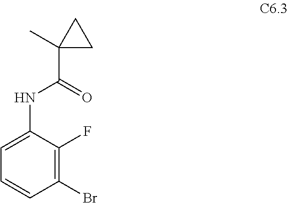

N-(3-bromo-2-fluorophenyl)cyclopropanecarboxamide (C6.4). C6.4 was prepared from 3-bromo-2-fluoro-aniline using Step 1 in the synthesis of C6.1 and using cyclopropanecarbonyl chloride instead of 2-bromo-2-methyl-propanoyl bromide.

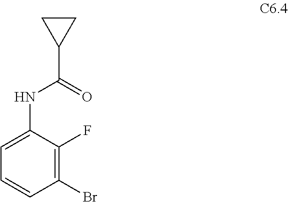 [SMILES: O=C(Nc1cccc(Br)c1F)C1CC1]

### Preparation of dimethyl((3-(4,4,5,5-tetramethyl-1,3,2-dioxaborolan-2-yl)phenyl)imino)-λ⁶-sulfanone (C7.1)

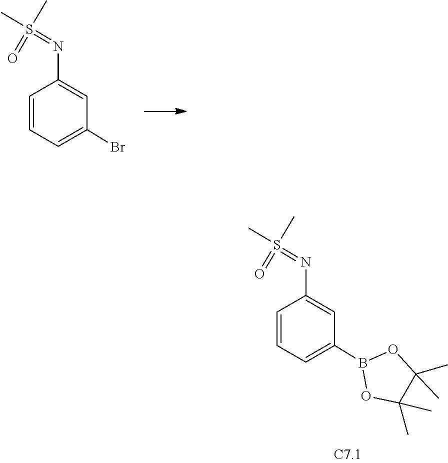

A vial was charged with ((3-bromophenyl)imino)dimethyl-λ⁶-sulfanone (60 mg, 0.24 mmol), B₂pin₂ (92 mg, 0.36 mmol), KOAc (71 mg, 0.73 mmol), and Pd(dppf)Cl₂-DCM (20 mg, 0.024 mmol). 1,4-Dioxane (3 mL) was added and the mixture was degassed by sparging with argon for 5 minutes. The mixture was stirred at 110° C. for 3 hours. The mixture was cooled to ambient temperature, filtered over a pad of celite, and rinsed with EtOAc. The organics were concentrated and subjected to flash column chromatography (SiO₂, hexanes-EtOAc) to yield dimethyl((3-(4,4,5,5-tetramethyl-1,3,2-dioxaborolan-2-yl)phenyl)imino)-λ⁶-sulfanone (C7.1). ES/MS m/z: 296.1 [M+H]⁺.

(2-cyano-3-fluoropyridin-4-yl)boronic acid (C7.2). C7.2 was prepared analogously to C7.1 using C1.3.

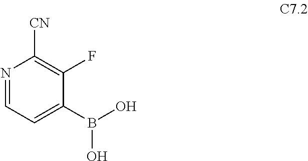 [SMILES: N#Cc1nccc(B(O)O)c1F]

(2-(ethoxycarbonyl)-1-methyl-1H-imidazol-4-yl)boronic acid (C7.3). C7.3 was prepared analogously to C7.1 using C1.1 and XPhos Pd G2 instead of Pd(dppf)Cl₂-DCM.

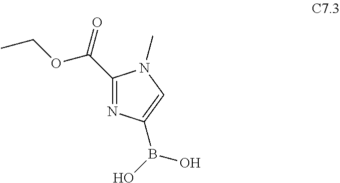

(2-(ethoxycarbonyl)-1-methyl-1H-imidazol-5-yl)boronic acid (C7.4). C7.4 was prepared analogously to C7.1 using C1.2 and Xphos Pd G2 instead of Pd(dppf)Cl₂-DCM.

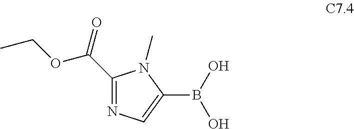

methyl 5-fluoro-1-(tetrahydro-2H-pyran-2-yl)-6-(4,4,5,5-tetramethyl-1,3,2-dioxaborolan-2-yl)-1H-indazole-4-carboxylate (C7.5). C7.5 was prepared analogously to C7.1 using C2.1. ES/MS m/z 404.9 [M+H]+.

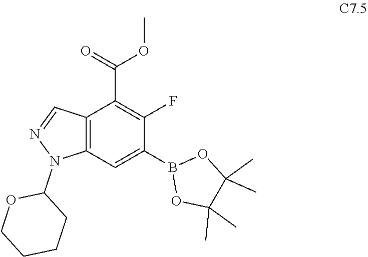 [SMILES: COC(=O)c1c(F)c(B2OC(C)(C)C(C)(C)O2)cc2c1cnn2C1CCCCO1]

7-fluoro-6-(4,4,5,5-tetramethyl-1,3,2-dioxaborolan-2-yl)benzo[d]oxazol-2(3H)-one (C7.6) C7.6 was prepared analogously to C7.1 using C1.16 and using Pd₂dba₃ and PCy₃ instead of Pd(dppf)Cl₂-DCM. ES/MS m/z 279.8 [M+H]+.

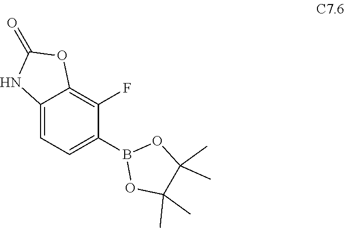 [SMILES: CC1(C)OB(c2ccc3[nH]c(=O)oc3c2F)OC1(C)C]

(5-((dimethyl(oxo)-λ⁶-sulfaneylidene)amino)-4-fluoropyridin-3-yl)boronic acid (C7.7). C7.7 was prepared analogously to C7.1 using C1.5

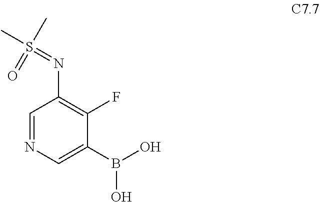

methyl 3-fluoro-4-(4,4,5,5-tetramethyl-1,3,2-dioxaborolan-2-yl)thiophene-2-carboxylate (C7.8). C7.8 was prepared analogously to C7.1 using C1.6.

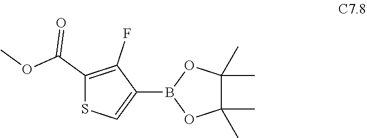

methyl 4-fluoro-5-(4,4,5,5-tetramethyl-1,3,2-dioxaborolan-2-yl)thiophene-3-carboxylate (C7.9). C7.9 was prepared analogously to C7.1 using C1.6.

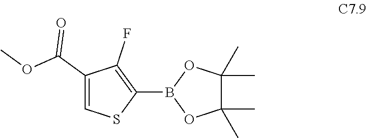

N-(2-fluoro-3-(4,4,5,5-tetramethyl-1,3,2-dioxaborolan-2-yl)phenyl)-1-methylcyclopropane-1-carboxamide (C7.10) C7.10 was prepared analogously to C7.1 using C6.3.

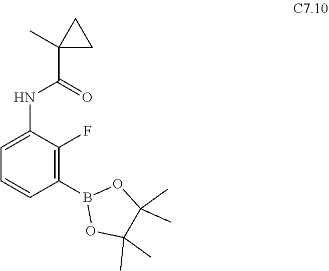 [SMILES: CC1(C(=O)Nc2cccc(B3OC(C)(C)C(C)(C)O3)c2F)CC1]

N-(2-fluoro-3-(4,4,5,5-tetramethyl-1,3,2-dioxaborolan-2-yl)phenyl)-2-hydroxy-2-methylpropanamide (C7.11) C7.11 was prepared analogously to C7.1 using C6.1.

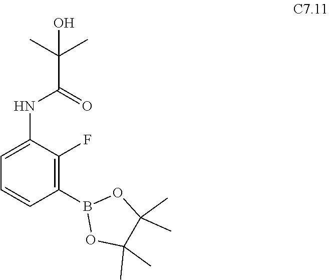 [SMILES: CC(C)(O)C(=O)Nc1cccc(B2OC(C)(C)C(C)(C)O2)c1F]

N-(2-fluoro-3-(4,4,5,5-tetramethyl-1,3,2-dioxaborolan-2-yl)phenyl)acetamide (C7.12) C7.12 was prepared analogously to C7.1 using C6.2.

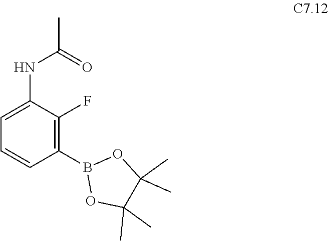 [SMILES: CC(=O)Nc1cccc(B2OC(C)(C)C(C)(C)O2)c1F]

N-(2-fluoro-3-(4,4,5,5-tetramethyl-1,3,2-dioxaborolan-2-yl)phenyl)cyclopropanecarboxamide (C7.13) C7.13 was prepared analogously to C7.1 using C6.4.

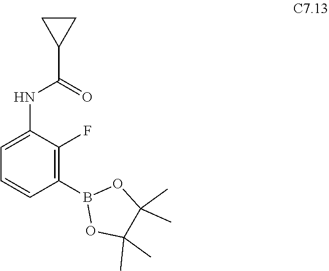 [SMILES: CC1(C)OB(c2cccc(NC(=O)C3CC3)c2F)OC1(C)C]

tert-butyl (2-fluoro-3-(4,4,5,5-tetramethyl-1,3,2-dioxaborolan-2-yl)phenyl)carbamate (C7.14) C7.14 was prepared analogously to C7.1 using C4.1.

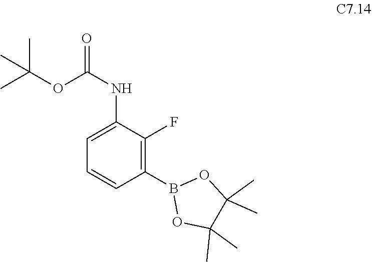 [SMILES: CC(C)(C)OC(=O)Nc1cccc(B2OC(C)(C)C(C)(C)O2)c1F]

4-fluoro-3,3-dimethyl-5-(4,4,5,5-tetramethyl-1,3,2-dioxaborolan-2-yl)indolin-2-one (C7.15). C7.15 was prepared analogously to C7.1 using C3.1 and using Pd₂dba₃ and PCy₃ instead of Pd(dppf)Cl₂-DCM. ES/MS m/z 306.8 [M+H]+.

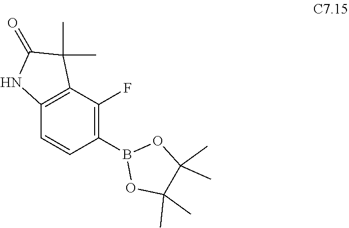 [SMILES: CC1(C)C(=O)Nc2ccc(B3OC(C)(C)C(C)(C)O3)c(F)c21]

5-fluoro-3-hydroxy-3-isopropyl-6-(4,4,5,5-tetramethyl-1,3,2-dioxaborolan-2-yl)-1-((2-(trimethylsilyl)ethoxy)methyl)indolin-2-one (C7.16). C7.16 was prepared analogously to C7.1 using C5.2.

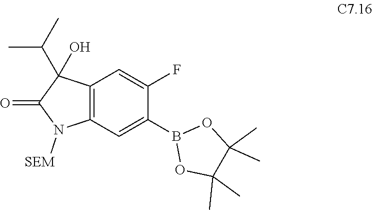 [SMILES: CC(C)C1(O)C(=O)N(COCC[Si](C)(C)C)c2cc(B3OC(C)(C)C(C)(C)O3)c(F)cc21]

3-hydroxy-3-isopropyl-6-(4,4,5,5-tetramethyl-1,3,2-dioxaborolan-2-yl)-4-(trifluoromethyl)-1-((2-(trimethylsilyl)ethoxy)methyl)indolin-2-one (C7.17). C7.17 was prepared analogously to C7.1 using C5.1.

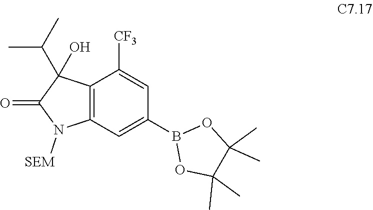 [SMILES: CC(C)C1(O)C(=O)N(COCC[Si](C)(C)C)c2cc(B3OC(C)(C)C(C)(C)O3)cc(C(F)(F)F)c21]

3-hydroxy-3-methyl-6-(4,4,5,5-tetramethyl-1,3,2-dioxaborolan-2-yl)indolin-2-one (C7.18). C7.18 was prepared analogously to C7.1 using C1.20.

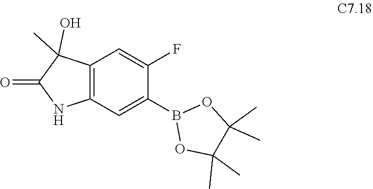 [SMILES: CC1(O)C(=O)Nc2cc(B3OC(C)(C)C(C)(C)O3)c(F)cc21]

5-fluoro-3-hydroxy-3-methyl-6-(4,4,5,5-tetramethyl-1,3,2-dioxaborolan-2-yl)indolin-2-one (C7.19). C7.19 was prepared analogously to C7.1 using C5.3.

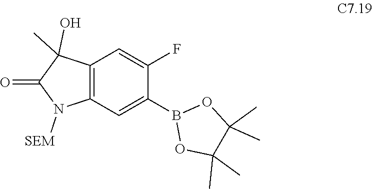 [SMILES: CC1(O)C(=O)N(COCC[Si](C)(C)C)c2cc(B3OC(C)(C)C(C)(C)O3)c(F)cc21]

3,3-difluoro-6-(4,4,5,5-tetramethyl-1,3,2-dioxaborolan-2-yl)-1-(2-trimethylsilylethoxymethyl)indolin-2-one (C7.20). C7.20 was prepared analogously to C7.1 using C5.4.

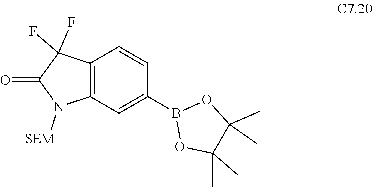 [SMILES: CC1(C)OB(c2ccc3c(c2)N(COCC[Si](C)(C)C)C(=O)C3(F)F)OC1(C)C]

1-(2-fluoro-3-(4,4,5,5-tetramethyl-1,3,2-dioxaborolan-2-yl)phenyl)-3-methylazetidin-3-ol (C7.21). C7.21 was prepared analogously to C7.1 using C8.1.

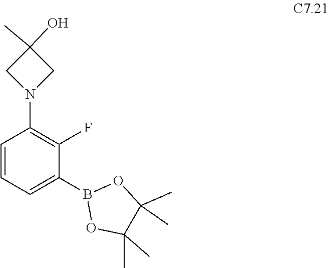 [SMILES: CC1(O)CN(c2cccc(B3OC(C)(C)C(C)(C)O3)c2F)C1]

(2-(3,5-dimethyl-1H-1,2,4-triazol-1-yl)-3-fluoropyridin-4-yl)boronic acid (C7.22). C7.22 was prepared analogously to C7.1 using C8.3 and toluene instead of 1,4-dioxane. ES/MS m/z: 237.2 [M+H]+

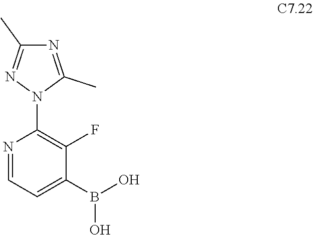 [SMILES: Cc1nc(C)n(-c2nccc(B(O)O)c2F)n1]

(3-fluoro-2-(3-methyl-1H-1,2,4-triazol-1-yl)pyridin-4-yl)boronic acid (C7.23). C7.23 was prepared analogously to C7.1 using C8.4 and toluene instead of 1,4-dioxane. ES/MS m/z: 222.9 [M+H]+

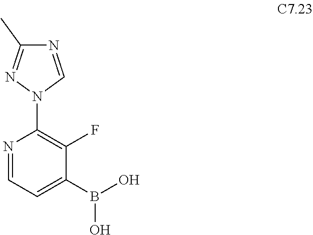 [SMILES: Cc1ncn(-c2nccc(B(O)O)c2F)n1]

(3-fluoro-2-(3-methyl-5-(trifluoromethyl)-1H-1,2,4-triazol-1-yl)pyridin-4-yl)boronic acid (C7.24). C7.24 was prepared analogously to C7.1 using C8.5 and toluene instead of 1,4-dioxane. ES/MS m/z: 290.8 [M+H]+

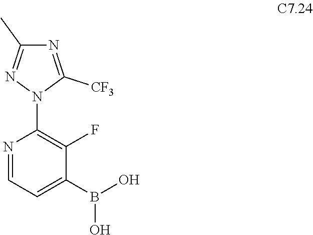 [SMILES: Cc1nc(C(F)(F)F)n(-c2nccc(B(O)O)c2F)n1]

(2-(3,5-dimethyl-1H-1,2,4-triazol-1-yl)pyrimidin-4-yl)boronic acid (C7.25). C7.25 was prepared analogously to C7.1 using C8.6 and toluene instead of 1,4-dioxane. ES/MS m/z: 219.9 [M+H]+

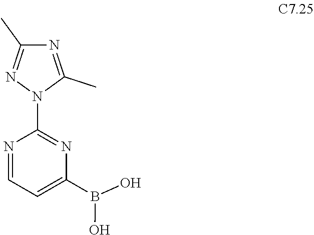 [SMILES: Cc1nc(C)n(-c2nccc(B(O)O)n2)n1]

1-(2-fluoro-6-(4,4,5,5-tetramethyl-1,3,2-dioxaborolan-2-yl)phenyl)-2,4-dimethyl-1H-imidazole (C7.26). C7.26 was prepared analogously to C7.1 using C8.8 and toluene instead of 1,4-dioxane. ES/MS m/z: 317.0 [M+H]+

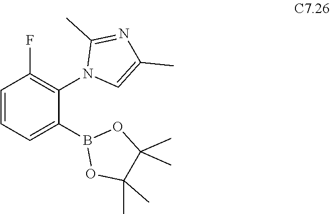 [SMILES: Cc1cn(-c2c(F)cccc2B2OC(C)(C)C(C)(C)O2)c(C)n1]

1-(2-fluoro-6-(4,4,5,5-tetramethyl-1,3,2-dioxaborolan-2-yl)phenyl)-3,5-dimethyl-1H-1,2,4-triazole (C7.27). C7.27 was prepared analogously to C7.1 using C8.9 and toluene instead of 1,4-dioxane. ES/MS m/z: 318.0 [M+H]+

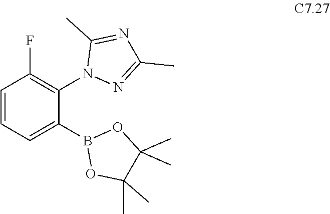 [SMILES: Cc1nc(C)n(-c2c(F)cccc2B2OC(C)(C)C(C)(C)O2)n1]

2-(3,5-dimethyl-4H-1,2,4-triazol-4-yl)-3-fluoro-4-(4,4,5,5-tetramethyl-1,3,2-dioxaborolan-2-yl)pyridine (C7.28). C7.28 was prepared analogously to C7.1 using C14.1 and toluene instead of 1,4-dioxane.

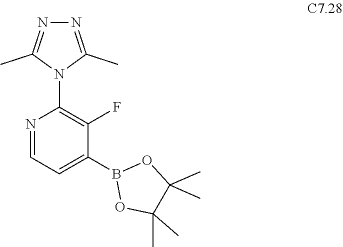

4-(2,6-difluoro-3-(4,4,5,5-tetramethyl-1,3,2-dioxaborolan-2-yl)phenyl)-3,5-dimethyl-4H-1,2,4-triazole (C7.29). C7.29 was prepared analogously to C7.1 using C14.3 and toluene instead of 1,4-dioxane. ES/MS m/z: 335.9 [M+H]+

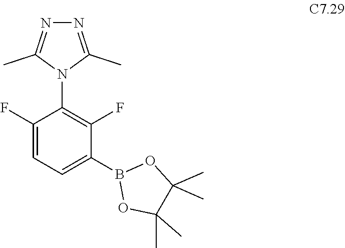

4-(2-fluoro-3-(4,4,5,5-tetramethyl-1,3,2-dioxaborolan-2-yl)phenyl)-3,5-dimethyl-4H-1,2,4-triazole (C7.30). C7.30 was prepared analogously to C7.1 using C14.4 and toluene instead of 1,4-dioxane. ES/MS m/z: 317.9 [M+H]+

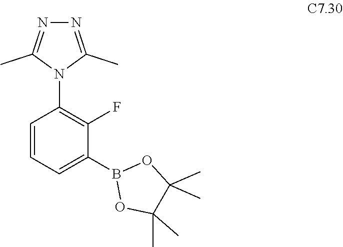

2-(2-chloro-3-(methylsulfonyl)phenyl)-4,4,5,5-tetramethyl-1,3,2-dioxaborolane (C7.31). C7.31 was prepared analogously to C7.1 using 1-bromo-2-chloro-3-(methylsulfonyl)benzene.

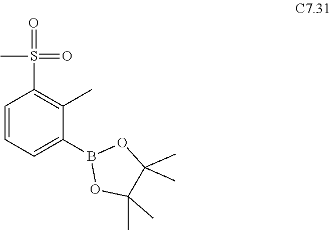

4,4,5,5-tetramethyl-2-(2-methyl-3-(methylsulfonyl)phenyl)-1,3,2-dioxaborolane (C7.32). C7.32 was prepared analogously to C7.1 using 1-bromo-2-methyl-3-(methylsulfonyl)benzene.

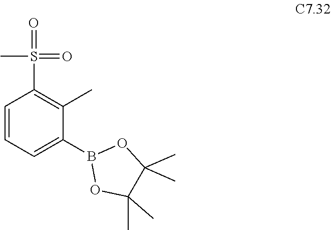

2-(4-chloro-2-fluoro-3-(methylsulfonyl)phenyl)-4,4,5,5-tetramethyl-1,3,2-dioxaborolane (C7.33). C7.33 was prepared analogously to C7.1 using 1-bromo-4-chloro-2-fluoro-3-(methylsulfonyl)benzene.

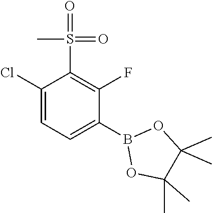 [SMILES: CC1(C)OB(c2ccc(Cl)c(S(C)(=O)=O)c2F)OC1(C)C]

4-(4,4,5,5-tetramethyl-1,3,2-dioxaborolan-2-yl)-2,3-dihydrobenzo[b]thiophene 1,1-dioxide (C7.33). C7.33 was prepared analogously to C7.1 using 4-bromo-2,3-dihydrobenzo[b]thiophene 1,1-dioxide.

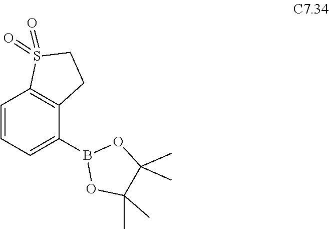 [SMILES: CC1(C)OB(c2cccc3c2CCS3(=O)=O)OC1(C)C]

### Preparation of 1-(4-bromo-3-fluoropyridin-2-yl)-3-methylazetidin-3-ol (C8.1)

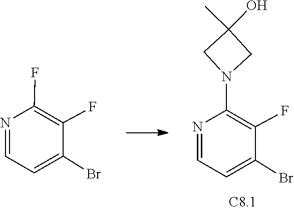

A vial was charged with 4-bromo-2,3-difluoropyridine (200 mg, 1.03 mmol), 3-methylazetidin-3-ol hydrochloride (153 mg, 1.24 mmol), MeCN (4.0 mL), and (iPr)₂EtN (0.54 mL, 3.10 mmol). The mixture was heated to 80° C. for 18 hrs. The mixture was concentrated and subjected to flash column chromatography (SiO₂, hexanes-EtOAc) to provide 1-(4-bromo-3-fluoropyridin-2-yl)-3-methylazetidin-3-ol (C8.1). 2-(3,5-dimethyl-1H-1,2,4-triazol-1-yl)-3-fluoro-6-(tributylstannyl)pyridine (C8.2). C8.2 was prepared analogously to C8.1 using 3,5-dimethyl-1H-1,2,4-triazole instead of 3-methylazetidin-3-ol hydrochloride, 6-bromo-2,3-difluoro-pyridine instead of 4-bromo-2,3-difluoropyridine, and Cs₂CO₃ instead of (iPr)₂EtN. ES/MS m/z: 271.0 [M+H]+

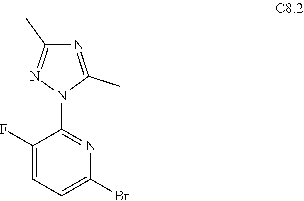

4-bromo-2-(3,5-dimethyl-1H-1,2,4-triazol-1-yl)-3-fluoropyridine (C8.3). C8.3 was prepared analogously to C8.1 using 3,5-dimethyl-1H-1,2,4-triazole instead of 3-methylazetidin-3-ol hydrochloride and Cs₂CO₃ instead of (iPr)₂EtN. ES/MS m/z: 271.0 [M+H]+

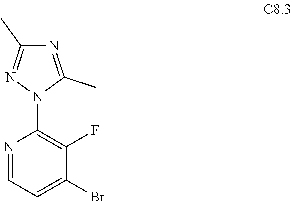

4-bromo-3-fluoro-2-(3-methyl-1H-1,2,4-triazol-1-yl)pyridine (C8.4). C8.4 was prepared analogously to C8.1 using 3-methyl-1H-1,2,4-triazole instead of 3-methylazetidin-3-ol hydrochloride, Cs₂CO₃ instead of (iPr)₂EtN, and DMF instead of MeCN.

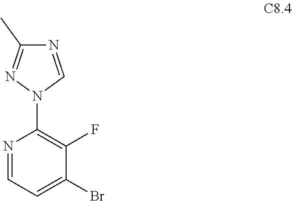

4-bromo-3-fluoro-2-(3-methyl-5-(trifluoromethyl)-1H-1,2,4-triazol-1-yl)pyridine (C8.5). C8.5 was prepared analogously to C8.1 using 3-methyl-5-(trifluoromethyl)-1H-1,2,4-triazole instead of 3-methylazetidin-3-ol hydrochloride and Cs₂CO₃ instead of (iPr)₂EtN.

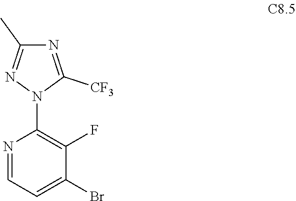 [SMILES: Cc1nc(C(F)(F)F)n(-c2nccc(Br)c2F)n1]

4-bromo-2-(3,5-dimethyl-1H-1,2,4-triazol-1-yl)pyrimidine (C8.6). C8.6 was prepared analogously to C8.1 using 3,5-dimethyl-1H-1,2,4-triazole instead of 3-methylazetidin-3-ol hydrochloride, 4-bromo-2-chloro-pyrimidine instead of 4-bromo-2,3-difluoropyridine, and Cs₂CO₃ instead of (iPr)₂EtN.

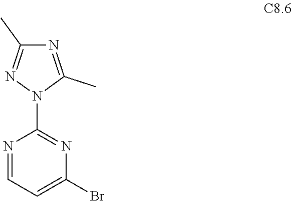

2-bromo-6-(3,5-dimethyl-1H-1,2,4-triazol-1-yl)pyridine (C8.7). C8.7 was prepared analogously to C8.1 using 3,5-dimethyl-1H-1,2,4-triazole instead of 3-methylazetidin-3-ol hydrochloride, 2-bromo-6-fluoro-pyridine instead of 4-bromo-2,3-difluoropyridine, and Cs₂CO₃ instead of (iPr)₂EtN.

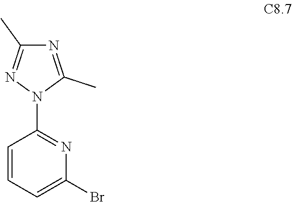

1-(2-bromo-6-fluorophenyl)-2,4-dimethyl-1H-imidazole (C8.8). C8.8 was prepared analogously to C8.1 using, 4-dimethyl-1H-imidazole instead of 3-methylazetidin-3-ol hydrochloride, 1-bromo-2,3<difluorobenzene instead of 4-bromo-2,3-difluoropyridine, and Cs₂CO₃ instead of (iPr)₂EtN.

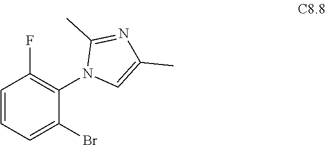

1-(2-bromo-6-fluorophenyl)-3,5-dimethyl-1H-1,2,4-triazole (C8.9). C8.9 was prepared analogously to C8.1 using 3,5-dimethyl-1H-1,2,4-triazole instead of 3-methylazetidin-3-ol hydrochloride, 1-bromo-2,3-difluorobenzene instead of 4-bromo-2,3-difluoropyridine, and Cs₂CO₃ instead of (iPr)₂EtN.

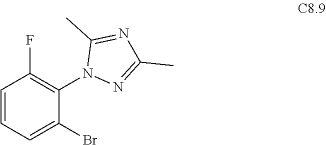

### Preparation of 5-(2-fluoro-3-(4,4,5,5-tetramethyl-1,3,2-dioxaborolan-2-yl)phenyl)-4-methylthiazole (C9.1)

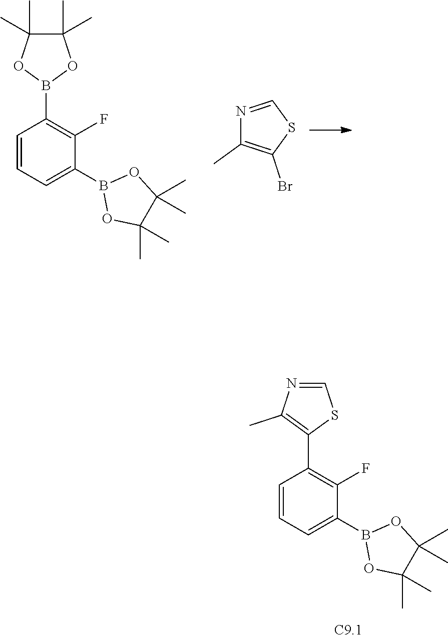

A vial was charged with 5-bromo-4-methylthiazole (37 mg, 0.21 mmol), 2,2′-(2-fluoro-1,3-phenylene)bis(4,4,5,5-tetramethyl-1,3,2-dioxaborolane) (289 mg, 0.84 mmol), Cs₂CO₃ (205 mg, 0.63 mmol), Pd(dppf)Cl₂-DCM (16 mg, 0.021 mmol), PhMe (1 mL), and water (0.2 mL). The mixture was heated to 80° C. for 18 hrs, after which it was cooled to ambient temperature, concentrated, and subjected to flash column chromatography (SiO₂, hexanes-EtOAc) to provide 5-(2-fluoro-3-(4,4,5,5-tetramethyl-1,3,2-dioxaborolan-2-yl)phenyl)-4-methylthiazole (C9.1). 4-(2-fluoro-3-(4,4,5,5-tetramethyl-1,3,2-dioxaborolan-2-yl)phenyl)-3-methylisothiazole (C9.2). C9.2 was prepared analogously to C9.1, using 4-bromo-3-methylisothiazole instead of 5-bromo-4-methylthiazole.

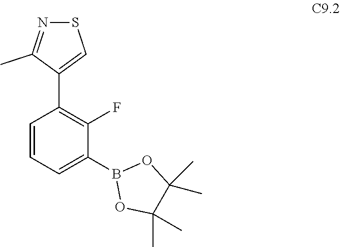

5-(2-fluoro-3-(4,4,5,5-tetramethyl-1,3,2-dioxaborolan-2-yl)phenyl)-1-methyl-1H-1,2,4-triazole (C9.3). C9.3 was prepared analogously to C9.1, using 5-bromo-1-methyl-1H-1,2,4-triazole instead of 5-bromo-4-methylthiazole.

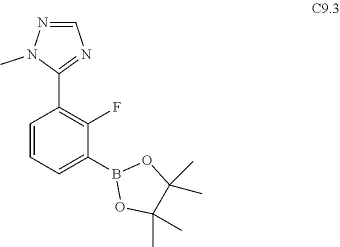

### Preparation of tert-butyl ((2-fluoro-3-(4,4,5,5-tetramethyl-1,3,2-dioxaborolan-2-yl)phenyl)(methyl)(oxo)-λ⁶-sulfaneylidene)carbamate (C10.1)

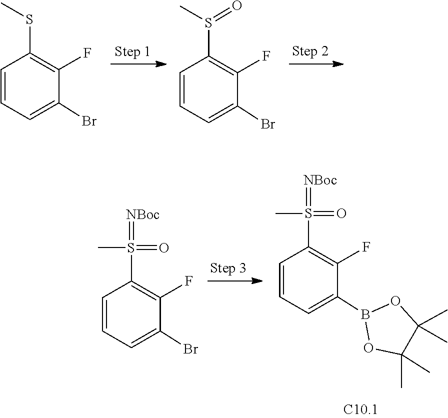

Step 1. A vial was charged with (3-bromo-2-fluorophenyl)(methyl)sulfane (500 mg, 2.26 mmol) and DCM (3.0 mL), followed by mCPBA (77%, 558 mg, 2.49 mmol). The mixture was stirred for 15 minutes, concentrated and subjected to flash column chromatography (SiO₂, hexanes-EtOAc) to provide 1-bromo-2-fluoro-3-(methylsulfinyl)benzene.

Step 2. A vial was charged with 1-bromo-2-fluoro-3-(methylsulfinyl)benzene (536 mg, 2.26 mmol), PhI(OAc)₂ (1.89 g, 5.87 mmol), tert-butyl carbamate (688 mg, 5.87 mmol), MgO (379 mg, 9.4 mmol), and rhodium acetate (26 mg, 0.050 mmol), followed by DCM (15 mL). The mixture was heated to 50° C. for 18 hours, after which it was filtered over celite, concentrated, and subjected to flash column chromatography (SiO₂, hexanes-EtOAc) to provide tert-butyl ((3-bromo-2-fluorophenyl)(methyl)(oxo)-λ⁶-sulfaneylidene)carbamate.

Step 3. ((2-fluoro-3-(4,4,5,5-tetramethyl-1,3,2-dioxaborolan-2-yl)phenyl)(methyl)(oxo)-λ⁶-sulfaneylidene)carbamate (C10.1) was prepared analogously to C7.1 using ((3-bromo-2-fluorophenyl)(methyl)(oxo)-λ⁶-sulfaneylidene)carbamate instead of ((3-bromophenyl)imino)dimethyl-λ⁶-sulfanone.

### tert-butyl (S)-((2-fluoro-3-(4,4,5,5-tetramethyl-1,3,2-dioxaborolan-2-yl)phenyl) (methyl)(oxo)-λ⁶-sulfaneylidene)carbamate and tert-butyl (R)-((2-fluoro-3-(4,4,5,5-tetramethyl-1,3,2-dioxaborolan-2-yl)phenyl)(methyl)(oxo)-λ⁶-sulfaneylidene)carbamate (C10.1a and C10.1b)

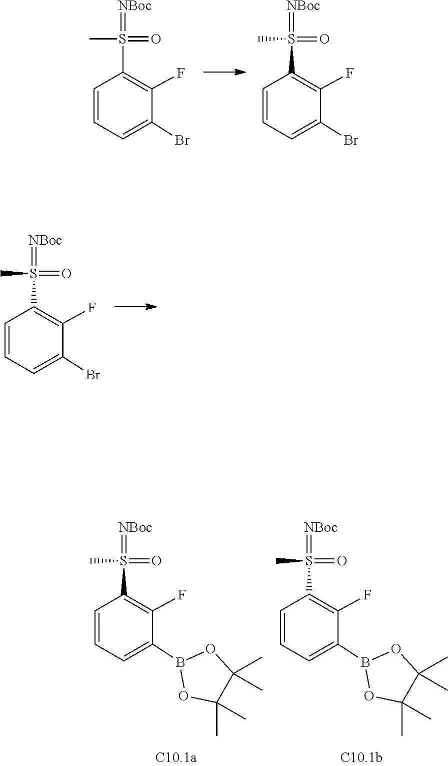

Stereoisomers of tert-butyl ((3-bromo-2-fluorophenyl)(methyl)(oxo)-λ⁶-sulfaneylidene)carbamate was separated by SFC. Stereochemistry was assigned arbitrarily. (CHIRALPAK IG (4.6×100 mm), 15% EtOH in CO₂, 3 mL/min, 40° C.). The individual stereoisomers were converted to C10.1a and C10.1b using Step 3 in the preparation of C10.1.

### Preparation of 1-(2-fluoro-3-(4,4,5,5-tetramethyl-1,3,2-dioxaborolan-2-yl)phenyl)-2,4-dimethyl-1H-imidazole (C11.1

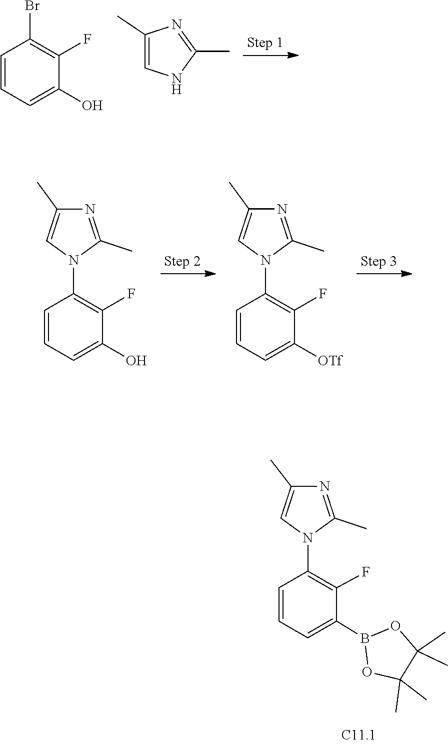

Step 1. A flask was charged with 3-bromo-2-fluorophenol (200 mg, 1.05 mmol), 2,4-dimethyl-1H-imidazole (130 mg, 1.36 mmol), 4,7-dimethoxy-1,10-phenanthroline (33 mg, 0.14 mmol), PEG 3350 (160 mg, 0.05 mmol), cesium carbonate (512 mg, 1.57 mmol), copper (I) oxide (15 mg, 0.11 mmol), and butyronitrile (4 mL). The mixture was heated to 120° C. and stirred for 24 h. The mixture was allowed to warm to room temperature and filtered over Celite. After filtration, the filtrate was concentrated under reduced pressure. The residue was used directly in the next step. ES/MS m/z: 207.2 [M+H]+

Step 2. A flask was charged with the crude residue from step 1, N-phenylbis(trifluoromethane)sulfonimide (449 mg, 1.26 mmol), triethylamine (0.29 mL, 2.09 mmol), and DMF (4.0 mL). The mixture was stirred at room temperature for 30 min. LiCl (sat. aq. 5 mL) was added and the resulting mixture was extracted with EtOAc (3×10 mL). The combined organic layers were dried over anhydrous Na₂SO₄. After filtration, the filtrate was concentrated under reduced pressure. The resulting residue was subject to flash column chromatography (SiO₂, hexane-EtOAc) to afford 3-(2,4-dimethyl-1H-imidazol-1-yl)-2-fluorophenyl trifluoromethanesulfonate. ES/MS m/z: 339.0 [M+H]+

Step 3. A vial was charged with 3-(2,4-dimethyl-1H-imidazol-1-yl)-2-fluorophenyl trifluoromethanesulfonate (152 mg, 0.45 mmol), bis(pinacoloato)diboron (160 mg, 0.63 mmol), PdCl₂(dppf) (33 mg, 0.04 mmol), potassium acetate (132 mg, 1.35 mmol) and toluene (4.6 mL). The mixture was sparged with argon for 5 min and then heated to 110° C. After 3 h, the mixture was allowed to cool to room temperature, filtered over Celite, and concentrated to afford 1-(2-fluoro-3-(4,4,5,5-tetramethyl-1,3,2-dioxaborolan-2-yl)phenyl)-2,4-dimethyl-1H-imidazole (C11.1). ES/MS m/z: 317.2 [M+H]+

1-(2-fluoro-3-(4,4,5,5-tetramethyl-1,3,2-dioxaborolan-2-yl)phenyl)-2-methyl-1H-imidazole (C11.2). C11.2 was prepared analogously to C11.1, using 2-methyl-1H-imidazole instead of 2,4-dimethyl-1H-imidazole. ES/MS m/z: 303.0 [M+H]+

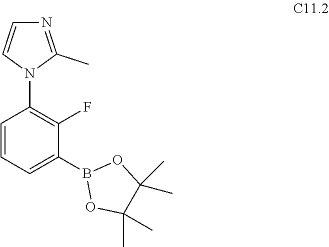 [SMILES: Cc1nccn1-c1cccc(B2OC(C)(C)C(C)(C)O2)c1F]

1-(2-fluoro-3-(4,4,5,5-tetramethyl-1,3,2-dioxaborolan-2-yl)phenyl)-4-methyl-1H-imidazole (C11.3). C11.3 was prepared analogously to C11.1, using 4-methyl-1H-imidazole instead of 2,4-dimethyl-1H-imidazole. ES/MS m/z: 302.9 [M+H]+

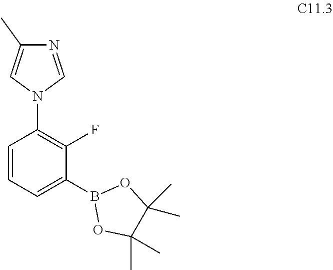

4-(2-fluoro-3-(4,4,5,5-tetramethyl-1,3,2-dioxaborolan-2-yl)phenyl)-3-methyl-4H-1,2,4-triazole (C11.4). C11.4 was prepared analogously to C11.1, using 3-methyl-4H-1,2,4-triazole instead of 2,4-dimethyl-1H-imidazole. ES/MS m/z: 317.9 [M+H]+

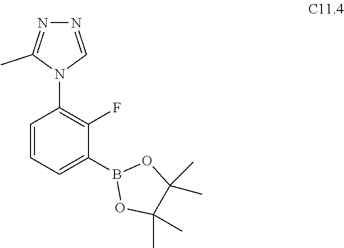

### Preparation of 5-(2-fluoro-3-(4,4,5,5-tetramethyl-1,3,2-dioxaborolan-2-yl)phenyl)-1,3-dimethyl-1H-1,2,4-triazole (C12.1)

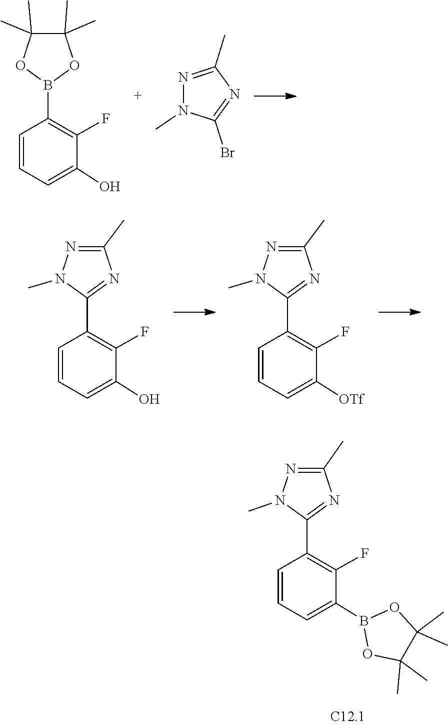

Step 1. 3-(1,3-dimethyl-1H-1,2,4-triazol-5-yl)-2-fluorophenol was prepared analogously to C9.1, using 2-fluoro-3-(4,4,5,5-tetramethyl-1,3,2-dioxaborolan-2-yl)phenol instead of 2,2′-(2-fluoro-1,3-phenylene)bis(4,4,5,5-tetramethyl-1,3,2-dioxaborolane), 5-bromo-1,3-dimethyl-1,2,4-triazole instead of 5-bromo-4-methylthiazole, and 1,4-dioxane instead of toluene. Crude residue used directly in next step. ES/MS m/z: 2008.0 [M+H]+

Step 2. 3-(1,3-dimethyl-1H-1,2,4-triazol-5-yl)-2-fluorophenyl trifluoromethanesulfonate was synthesized from 3-(1,3-dimethyl-1H-1,2,4-triazol-5-yl)-2-fluorophenol following the procedure described in Step 2 of C11.1. ES/MS m/z: 340.0 [M+H]+

Step 3. 5-(2-fluoro-3-(4,4,5,5-tetramethyl-1,3,2-dioxaborolan-2-yl)phenyl)-1,3-dimethyl-1H-1,2,4-triazole was synthesized from 3-(1,3-dimethyl-1H-1,2,4-triazol-5-yl)-2-fluorophenyl trifluoromethanesulfonate following the procedure described in Step 3 of C11.1. ES/MS m/z: 318.0 [M+H]+

4-(2-fluoro-3-(4,4,5,5-tetramethyl-1,3,2-dioxaborolan-2-yl)phenyl)-2,5-dimethyl-2H-1,2,3-triazole (C12.2). C12.2 was prepared analogously to C12.1, using 4-bromo-2,5-dimethyl-triazole instead of 5-bromo-1,3-dimethyl-1,2,4-triazole. ES/MS m/z: 318.2 [M+H]+

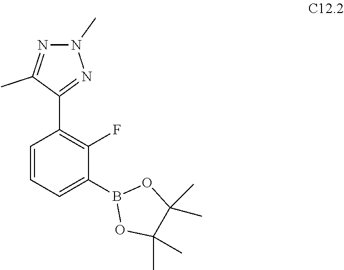

5-(2-fluoro-3-(4,4,5,5-tetramethyl-1,3,2-dioxaborolan-2-yl)phenyl)-1,3-dimethyl-1H-pyrazole (C12.3). C12.3 was prepared analogously to C12.1, using 5-bromo-1,3-dimethyl-pyrazole instead of 5-bromo-1,3-dimethyl-1,2,4-triazole. ES/MS m/z: 317.2 [M+H]+

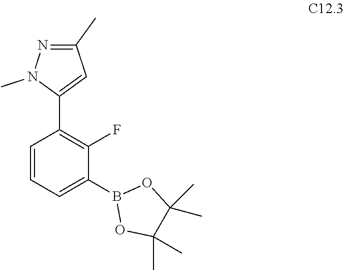

1-cyclopropyl-5-(2-fluoro-3-(4,4,5,5-tetramethyl-1,3,2-dioxaborolan-2-yl)phenyl)-1H-1,2,4-triazole (C12.4). C12.4 was prepared analogously to C12.1, using 5-bromo-1-cyclopropyl-1,2,4-triazole instead of 5-bromo-1,3-dimethyl-1,2,4-triazole.

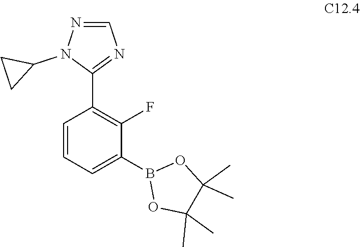 [SMILES: CC1(C)OB(c2cccc(-c3ncnn3C3CC3)c2F)OC1(C)C]

5-(2-fluoro-3-(4,4,5,5-tetramethyl-1,3,2-dioxaborolan-2-yl)phenyl)-2-methyl-2H-tetrazole (C12.5). C12.5 was prepared analogously to C12.1, using 5-bromo-2-methyl-tetrazole instead of 5-bromo-1,3-dimethyl-1,2,4-triazole.

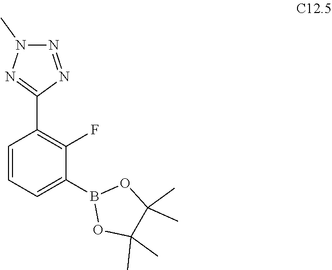

### Preparation of 1-(2-fluoro-3-(4,4,5,5-tetramethyl-1,3,2-dioxaborolan-2-yl)phenyl)-3,5-dimethyl-1H-pyrazole (C13.1)

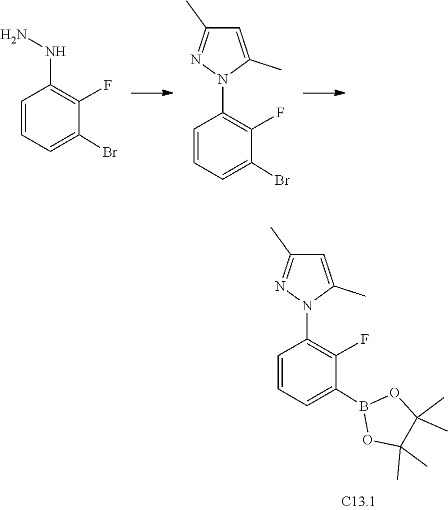

Step 1. A vial was charged with (3-bromo-2-fluoro-phenyl)hydrazine hydrochloride (0.15 g 0.62 mmol), pentane-2,4-dione (0.08 mL, 0.77 mmol), and AcOH (3 mL). The mixture was heated to 100° C. for 2 h. The mixture was then allowed to cool to room temperature, sat. aq. NaHCO₃ was added to the mixture, and the aqueous layer was extracted with EtOAc. The organic layer was dried (Na₂SO₄), filtered, and concentrated. The resulting residue was subject to flash column chromatography (SiO₂, hexane-EtOAc) to afford to afford 1-(3-bromo-2-fluoro-phenyl)-3,5-dimethyl-pyrazole. ES/MS m/z: 269.0 [M+H]+

Step 2. C13.1 was prepared analogously to C7.1 using 1-(3-bromo-2-fluoro-phenyl)-3,5-dimethyl-pyrazole and toluene instead of 1,4-dioxane. 1-(2-fluoro-3-(4,4,5,5-tetramethyl-1,3,2-dioxaborolan-2-yl)phenyl)-3,4,5-trimethyl-1H-pyrazole (C13.2). C13.2 was prepared analogously to C13.1, using 3-methylpentane-2,4-dione instead of pentane-2,4-dione. ES/MS m/z: 331.2 [M+H]+

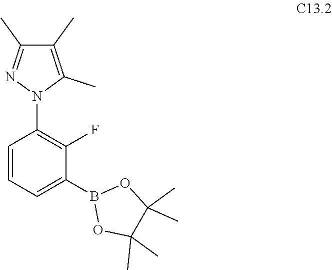

1-(2-fluoro-3-(4,4,5,5-tetramethyl-1,3,2-dioxaborolan-2-yl)phenyl)-3,5-dimethyl-1H-1,2,4-triazole (C13.3). C13.3 was prepared analogously to C13.1, using N-acetylacetamide instead of pentane-2,4-dione, pyridine instead of acetic acid, heating to 120° C. instead of 100° C., and concentrating mixture instead of aqueous workup for step 1. ES/MS m/z: 317.9 [M+H]+

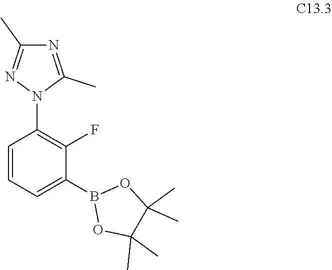

### Preparation of 4-bromo-2-(3,5-dimethyl-4H-1,2,4-triazol-4-yl)-3-fluoropyridine (C14.1)

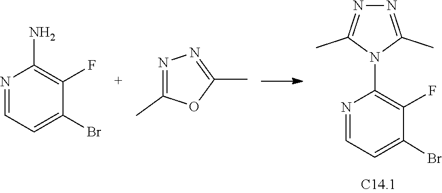

A flask was charged with 2,5-dimethyl-1,3,4-oxadiazole (0.51 g, 5.24 mmol), 4-bromo-3-fluoro-pyridin-2-amine (0.50 g, 2.62 mmol) and p-toluene sulfonic acid monohydrate (50 mg. 0.26 mmol). The mixture was heated to 140° C. after 16 h, the mixture was allowed to cool to room temperature, filtered over Celite, and concentrated. The resulting residue was subject to flash column chromatography (SiO₂, hexane-EtOAc) to afford 4-bromo-2-(3,5-dimethyl-4H-1,2,4-triazol-4-yl)-3-fluoropyridine (C14.1). ES/MS m/z: 270.8 [M+H]+

6-bromo-2-(3,5-dimethyl-4H-1,2,4-triazol-4-yl)-3-fluoropyridine (C14.2). C14.2 was prepared analogously to C14.1, using 6-bromo-3-fluoro-pyridin-2-amine instead of 4-bromo-3-fluoro-pyridin-2-amine. ES/MS m/z: 270.8 [M+H]+

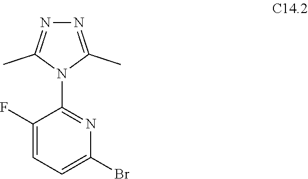

4-(3-bromo-2,6-difluorophenyl)-3,5-dimethyl-4H-1,2,4-triazole (C14.3). C143 was prepared analogously to C14.1, using 3-bromo-2,6-difluoro-aniline instead of 4-bromo-3-fluoro-pyridin-2-amine. ES/MS m/z: 287.8 [M+H]+

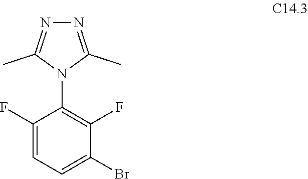

4-(3-bromo-2-fluorophenyl)-3,5-dimethyl-4H-1,2,4-triazole (C14.4). C14.4 was prepared analogously to C14.1, using 3-bromo-2-fluoro-aniline instead of 4-bromo-3-fluoro-pyridin-2-amine. ES/MS m/z: 271.8 [M+H]+

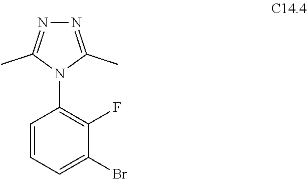

### Preparation of 1-(difluoromethyl)-5-(2-fluoro-3-(4,4,5,5-tetramethyl-1,3,2-dioxaborolan-2-yl)phenyl)-1H-1,2,4-triazole (C15.1)

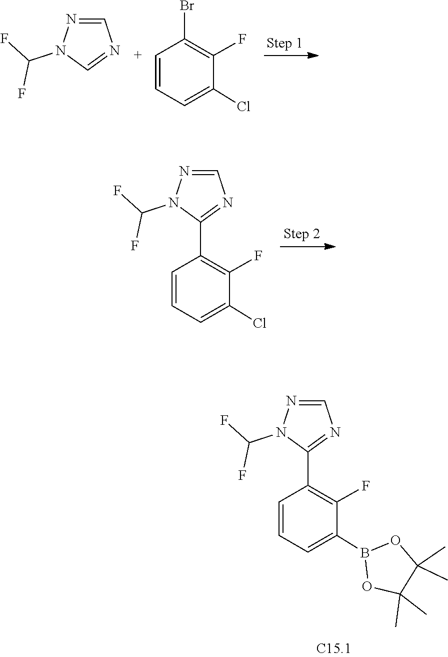

Step 1. A mixture of 1-(difluoromethyl)-1H-1,2,4-triazole (2.05 g, 17.2 mmol), 1-bromo-3-chloro-2-fluorobenzene (3.00 g, 14.3 g), pivalic acid (0.439 g, 4.30 mmol), potassium carbonate (5.98 g, 43.0 mmol), cataCXium Pd G4 (0.532 g, 0.716 mmol) and cataCXium A (0.257 g, 0.716 mmol) in toluene (14.4 mL) was sparged with Ar for several minutes and then was stirred at 120° C. for 16 h. The mixture was filtered through a pad of Celite. The filtrate was concentrated and the residue was subjected to column chromatography on silica gel to afford 5-(3-chloro-2-fluorophenyl)-1-(difluoromethyl)-TH-1,2,4-triazole. ES/MS: m/z 248.0 [M+H]+.

Step 2. A mixture of 5-(3-chloro-2-fluorophenyl)-1-(difluoromethyl)-1H-1,2,4-triazole (0.650 g, 2.07 mmol), bis(pinacolato)diboron (0.790 g, 3.11 mmol), potassium acetate (0.611 g, 6.22 mmol), Pd₂(dba)₃ (0.095 g, 0.104 mmol) and XPhos (0.102 g, 0.207 mmol) in 1,4-dioxane (10.5 mL) was sparged with N₂ for several minutes and was then stirred at 100° C. for 2 h. The mixture was cooled to rt and filtered through a pad of Celite. The filtrate was concentrated and the residue containing 1-(difluoromethyl)-5-(2-fluoro-3-(4,4,5,5-tetramethyl-1,3,2-dioxaborolan-2-yl)phenyl)-1H-1,2,4-triazole (C15.1) was taken on crude. ES/MS: m/z 258.0 [M+H]⁺ (corresponds to the analogous boronic acid).

### Preparation of 2-(3,5-dimethyl-1H-1,2,4-triazol-1-yl)-3-fluoro-6-(tributylstannyl)pyridine (C16.1)

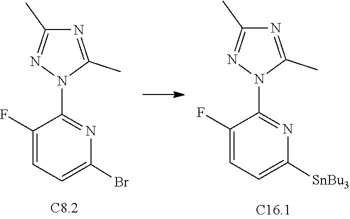

A flask was charged with 4-bromo-2-(3,5-dimethyl-1,2,4-triazol-1-yl)-3-fluoro-pyridine (C8.2) (0.50 g, 1.84 mmol), bis(tricyclohexylphosphine)palladium(0) (0.25 g, 0.37 mmol), LiCl (0.39 g, 9.22 mmol), and 1,4-dioxane (17 mL). The mixture was sparged with argon for 5 min followed by addition of bis(tributyltin) (2.8 mL, 5.53 mmol). The mixture was heated to 110° C. and stirred for 20 h. The mixture was allowed to cool to room temperature, filtered over Celite, and concentrated to afford 2-(3,5-dimethyl-1H-1,2,4-triazol-1-yl)-3-fluoro-4-(tributylstannyl)pyridine (C16.1). ES/MS m/z: 483.0 [M+H]+

2-(3,5-dimethyl-1H-1,2,4-triazol-1-yl)-6-(tributylstannyl)pyridine (C16.2). C16.2 was prepared analogously to C16.1, using C8.7 instead of C8.2.

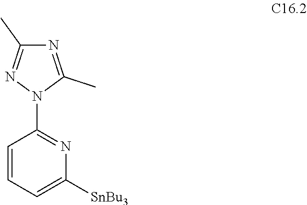

2-(3,5-dimethyl-1H-1,2,4-triazol-1-yl)-3-fluoro-4-(tributylstannyl)pyridine (C16.3). C16.3 was prepared analogously to C16.1, using C8.3 instead of C8.2.

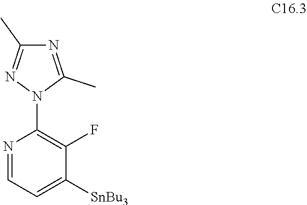

2-(3,5-dimethyl-4H-1,2,4-triazol-4-yl)-3-fluoro-6-(tributylstannyl)pyridine (C16.4). C16.4 was prepared analogously to C16.1, using C14.2 instead of C8.2. ES/MS m/z: 482.8 [M+H]+

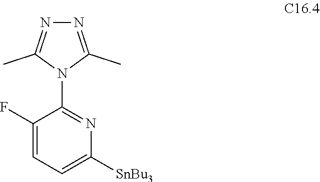

### Preparation of N-(2-(2-fluoro-3-(4,4,5,5-tetramethyl-1,3,2-dioxaborolan-2-yl)phenyl)propan-2-yl)acetamide (C17.1)

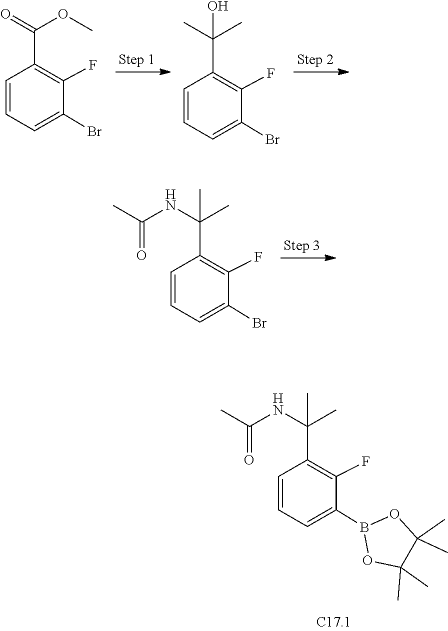 [SMILES: COC(=O)c1cccc(Br)c1F ; CC(C)(O)c1cccc(Br)c1F ; CC(=O)NC(C)(C)c1cccc(Br)c1F ; CC(=O)NC(C)(C)c1cccc(B2OC(C)(C)C(C)(C)O2)c1F]

Step 1. To a solution of methyl 3-bromo-2-fluoro-benzoate (408 mg, 1.75 mmol) in THF (4 mL) at −78° C. was added dropwise a solution of MeMgBr (3M in diethyl ether, 2.33 mL 7.00 mmol). The reaction mixture was warmed up to room temperature and stirred for 2 h. The solution was poured to an aqueous saturated solution of ammonium chloride and the organic material was extracted with EtOAc. The organic layer was dried over Na₂SO₄, filtered and concentrated to give 2-(3-bromo-2-fluoro-phenyl)propan-2-ol. 1HNMR (400 MHz, Chloroform-d) δ 7.57-7.51 (m, 1H), 7.45 (ddd, J=8.0, 6.4, 1.7 Hz, 1H), 7.01 (td, J=7.9, 1.0 Hz, 1H), 1.65 (d, J=1.2 Hz, 6H).

Step 2. To a solution of 2-(3-bromo-2-fluoro-phenyl)propan-2-ol (389 mg, 1.67 mmol) in acetonitrile (1 mL) was added acetic acid (0.1 mL). The reaction mixture was cooled to 0° C. and H₂SO₄ (0.1 mL) was added dropwise. The reaction mixture was warned to room temperature and stirred for 18 h. The reaction was then poured into ice and extracted with EtOAc. The organic layer was washed with saturated sodium bicarbonate, dried over Na₂SO₄, filtered and concentrated. The residue was subject to silica gel chromatography (0-100% EtOAc in hexane) to provide N-(2-(3-bromo-2-fluorophenyl)propan-2-yl)acetamide. 1HNMR (400 MHz, Chloroform-d) δ 7.43 (ddd, J=8.0, 6.4, 1.6 Hz, 1H), 7.32 (td, J=7.8, 1.6 Hz, 1H), 6.98 (td, J=8.0, 1.1 Hz, 1H), 5.83 (s, 1H), 1.96 (s, 3H), 1.73 (d, J=0.9 Hz, 6H).

Step 3. C17.1 was prepared analogously to C7.1 using N-(2-(3-bromo-2-fluorophenyl)propan-2-yl)acetamide.

### Preparation of 4-((2-fluoro-3-(4,4,5,5-tetramethyl-1,3,2-dioxaborolan-2-yl)phenyl)imino)-1,4λ⁶-oxathiane 4-oxide (C18.1)

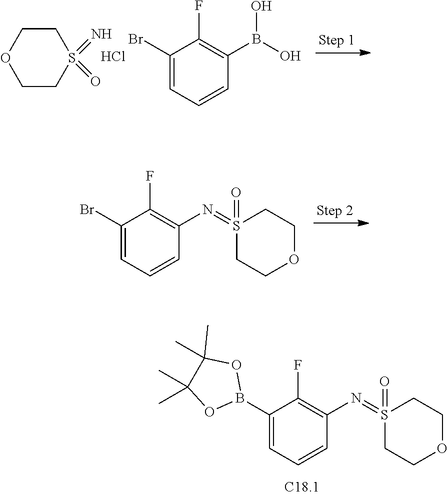

Step 1. A vial was charged with 4-imino-1,4,6-oxathiane 4-oxide hydrochloride (235 mg, 1.37 mmol), (3-bromo-2-fluoro-phenyl)boronic acid (250 mg, 1.14 mmol), and potassium carbonate (189 mg, 1.37 mmol. To it, DMF (9.2 mL) was added followed by copper(II) acetate (20.8 mg, 0.11 mmol), and the mixture was stirred open to the air at ambient temperature for 16 hours. It was partitioned between EtOAc and water, and the organic layer was collected and washed with 1M ammonium hydroxide and saturated sodium chloride. It was dried over anhydrous sodium sulfate, filtered, and concentrated. It was purified via flash chromatography (SiO₂, 5-100% ethyl acetate/hexanes linear gradient) to yield 4-((3-bromo-2-fluorophenyl)imino)-1,4λ⁶-oxathiane4-oxide. ¹H NMR (400 MHz, CDCl₃) δ 7.23-7.13 (m, 2H), 6.88 (td, J=8.1, 1.4 Hz, 1H), 4.25-4.07 (m, 4H), 3.47-3.34 (m, 2H), 3.28 (ddd, J=13.6, 9.0, 4.1 Hz, 2H).

Step 2. C18.1 was prepared analogously to C7.1 using 4-((3-bromo-2-fluorophenyl)imino)-1,4λ⁶-oxathiane 4-oxide.

N′-(2-fluoro-3-(4,4,5,5-tetramethyl-1,3,2-dioxaborolan-2-yl)phenyl)-N,N-dimethylmethanesulfonimidamide (C18.2) Prepared analogously to C18.1 using N-methyl-N-(methylsulfonimidoyl)methanamine hydrochloride instead of 4-imino-1,4λ⁶-oxathiane 4-oxide hydrochloride. ¹H NMR (400 MHz, CDCl) δ 7.31-7.23 (m, 1H), 7.12 (ddd, J=7.8, 6.1, 1.6 Hz, 1H), 6.84 (td, J=8.1, 1.5 Hz, 1H), 3.01 (s, 3H), 2.89 (s, 6H).

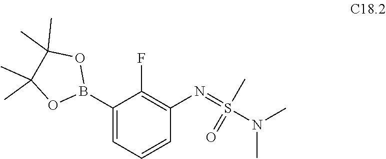

1-((2-fluoro-3-(4,4,5,5-tetramethyl-1,3,2-dioxaborolan-2-yl)phenyl)imino)tetrahydro-1H-1λ⁶-thiophene 1-oxide (C18.3) Prepared analogously to C18.1 using 1-iminotetrahydro-1H-1λ⁶-thiophene 1-oxide instead of 4-imino-1,4λ⁶-oxathiane 4-oxide hydrochloride and omitting potassium carbonate. ¹H NMR (400 MHz, CDCl₃) δ 7.18-7.07 (m, 2H), 6.93-6.83 (m, 1H), 3.49-3.34 (m, 2H), 3.28-3.13 (m, 2H), 2.42-2.21 (m, 4H).

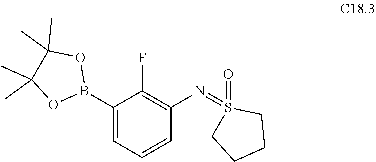 [SMILES: CC1(C)OB(c2cccc(N=S3(=O)CCCC3)c2F)OC1(C)C]

((2-fluoro-3-(4,4,5,5-tetramethyl-1,3,2-dioxaborolan-2-yl)phenyl)imino)dimethyl-λ⁶-sulfanone (C18.4) Prepared analogously to C18.1 using iminodimethyl-λ⁶-sulfanone instead of 4-imino-1,4λ⁶-oxathiane 4-oxide hydrochloride and omitting potassium carbonate. ES/MS m/z: 266.0 (M+H)⁺.

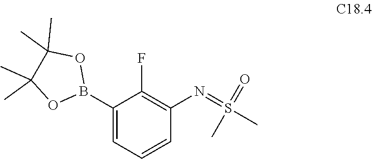

### Preparation of 2-(3-(cyclopropylsulfonyl)-2-fluorophenyl)-4,4,5,5-tetramethyl-1,3,2-dioxaborolane (C19.1)

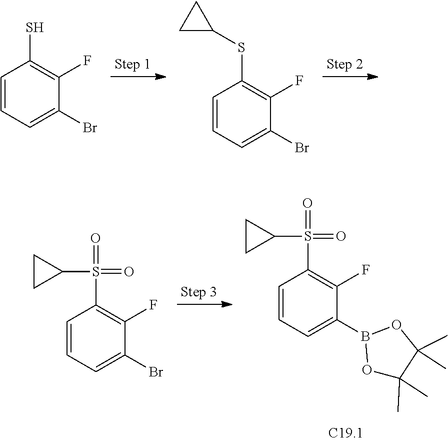 [SMILES: Fc1c(S)cccc1Br ; Fc1c(Br)cccc1SC1CC1 ; O=S(=O)(c1cccc(Br)c1F)C1CC1 ; CC1(C)OB(c2cccc(S(=O)(=O)C3CC3)c2F)OC1(C)C]

Step 1. To a solution of 3-bromo-2-fluoro-benzenethiol (120 mg, 0.580 mmol) and iodocyclopropane (195 mg, 1.16 mmol) in DMSO (0.5 mL) was added cesium carbonate (380 mg, 1.16 mmol) and stirred at 70° C. for 4 days. To the reaction mixture was added brine and the aqueous layer was washed with Et₂O three times. The combined organic layers were washed with brine twice and dried over Na₂SO₄, filtered and concentrated. The residue was purified by silica gel chromatography (0-50% EtOAc in hexane) to give (3-bromo-2-fluorophenyl)(cyclopropyl)sulfane. 1H NMR (400 MHz, Chloroform-d) δ 7.43 (ddd, J=8.0, 6.6, 1.6 Hz, 1H), 7.34 (ddd, J=7.9, 6.4, 1.6 Hz, 1H), 6.99 (td, J=8.0, 1.1 Hz, 1H), 2.18 (ttd, J=7.3, 4.3, 0.6 Hz, 1H), 1.12-1.04 (m, 2H), 0.75-0.66 (m, 2H).

Step 2. To a solution of (3-bromo-2-fluorophenyl)(cyclopropyl)sulfane (46 mg, 0.186 mmol) in dichloromethane (1.7 mL) at 0° C. was added 3-chloroperoxybenzoic acid, (167 mg, 0.745 mmol) and the reaction was allowed to warm to room temp and stirred overnight. Saturated sodium bicarbonate was added and the aqueous layer was washed with dichloromethane twice. The combined organic layers were dried over sodium sulfate, filtered, and concentrated. The residue was purified by silica gel chromatography (0-50% EtOAc in hex) to give 1-bromo-3-(cyclopropylsulfonyl)-2-fluorobenzene. 1H NMR (400 MHz, Chloroform-d) δ 7.88-7.79 (m, 2H), 7.21 (td, J=7.9, 1.0 Hz, 1H), 2.76 (ttd, J=8.0, 4.8, 1.7 Hz, 1H), 1.45-1.36 (m, 2H), 1.15-1.07 (m, 2H).

Step 3. C19.1 was prepared in analogy to was prepared analogously to C7.1 using 1-bromo-3-(cyclopropylsulfonyl)-2-fluorobenzene.

### Preparation of 2-(3-((difluoromethyl)sulfonyl)-2-fluorophenyl)-4,4,5,5-tetramethyl-1,3,2-dioxaborolane (C19.2)

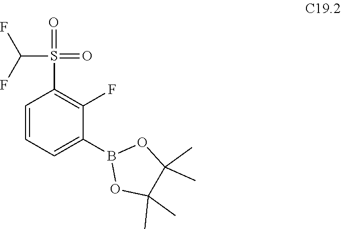 [SMILES: CC1(C)OB(c2cccc(S(=O)(=O)C(F)F)c2F)OC1(C)C]

Step 1: A solution of 3-bromo-2-fluoro-benzenethiol (154 mg, 0.744 mmol), sodium chlorodifluoroacetate (171 mg, 1.12 mmol), and potassium carbonate (206 mg, 1.49 mmol) in DMF (1.3 mL) was stirred at 130° C. for 1 hr. The reaction mixture was cooled to room temp and diluted with water. The aqueous layer was washed with Et₂O twice. The combined organic layer was washed with 10% citric acid solution, brine and dried over sodium sulfate, filtered and concentrated. The residue was purified by silica gel chromatography (0-50% EtOAc in hexane) to give (3-bromo-2-fluorophenyl)(difluoromethyl)sulfane. 1HNMR (400 MHz, Chloroform-d) δ 7.65 (ddd, J=8.0, 6.3, 1.7 Hz, 1H), 7.54 (ddd, J=7.9, 6.3, 1.7 Hz, 1H), 7.07 (td, J=7.9, 1.1 Hz, 1H), 6.89 (t, J=56.7 Hz, 1H).

C19.2 was prepared in analogy to Steps 2 and 3 for C19.1.

### Preparation of 1-((2-fluoro-3-(4,4,5,5-tetramethyl-1,3,2-dioxaborolan-2-yl)phenyl)sulfonyl)-2-methylpropan-2-ol (C19.3)

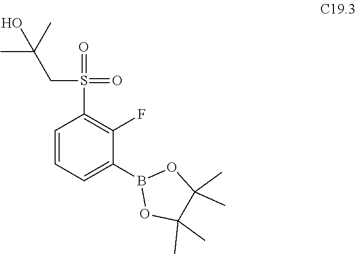 [SMILES: CC(C)(O)CS(=O)(=O)c1cccc(B2OC(C)(C)C(C)(C)O2)c1F]

Step 1: A solution of 3-bromo-2-fluoro-benzenethiol (132 mg, 0.637 mmol), 1-bromo-2-methyl-propan-2-ol (148 mg, 0.966 mmol) and potassium carbonate (176 mg, 1.27 mmoL) in acetone (0.6 mL) was stirred overnight and concentrated. To the residue was added water and DCM. The aqueous layer was washed twice with DCM. The combined organic layers were dried over sodium sulfate, filtered and concentrated to give 1-((3-bromo-2-fluorophenyl)thio)-2-methylpropan-2-ol. 1H NMR (400 MHz, Chloroform-d) δ 7.45-7.37 (m, 2H), 6.96 (td, J=8.0, 1.1 Hz, 1H), 3.10 (s, 2H), 1.32 (s, 6H).

C19.3 was prepared in analogy to Steps 2 and 3 for C19.1.

### Preparation of (2-fluoro-3-(1-oxido-4-oxo-3,4-dihydro-2H-1λ⁶,2,5-thiadiazol-1-yl)phenyl)boronic acid (C20.1)

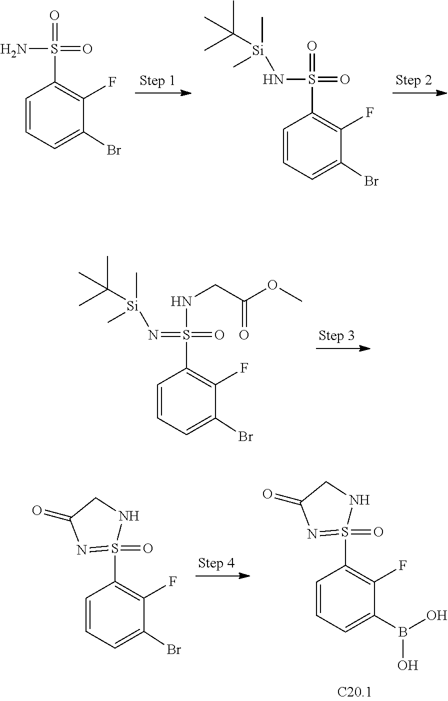 [SMILES: NS(=O)(=O)c1cccc(Br)c1F ; CC(C)(C)[Si](C)(C)NS(=O)(=O)c1cccc(Br)c1F ; COC(=O)CNS(=O)(=N[Si](C)(C)C(C)(C)C)c1cccc(Br)c1F ; O=C1CNS(=O)(c2cccc(Br)c2F)=N1 ; O=C1CNS(=O)(c2cccc(B(O)O)c2F)=N1]

Step 1. To a solution of 3-bromo-2-fluorobenzenesulfonamide (9.3 g, 0.0366 mol) and triethylamine (11.1 g, 0.110 mol) in dichloromethane (120 mL) was added tert-butyldimethylsilyl chloride (8.3 g, 0.0548 mol). The mixture was stirred at 25° C. for 1 hr. The mixture was concentrated under reduced pressure to give a residue. The residue was purified by prep-HPLC (column: Welch Xtimate C18 250*100 mm #0 um; mobile phase: [A: H2O (10 mM NH4HCO3); B: ACN]; B %: 60.00%-90.00%) to give 3-bromo-N-(tert-butyldimethylsilyl)-2-fluorobenzenesulfonamide. 1H NMR (400 MHz, Chloroform-d) δ=7.87-7.81 (m, 1H), 7.74 (ddd, J=1.6, 6.5, 7.9 Hz, 1H), 7.15 (t, J=7.9 Hz, 1H), 4.67 (s, 1H), 0.91 (s, 9H), 0.18 (s, 6H)

Step 2. A mixture of 3-bromo-N-(tert-butyldimethylsilyl)-2-fluorobenzenesulfonamide (8.0 g, 0.0217 mol) and triethylamine (6.6 g, 0.0651 mol) in dichloromethane (80.0 mL) was cooled to 0° C. under nitrogen atmosphere. Then a solution of dichlorotriphenylphosphorane (21.7 g, 0.0652 mol) in dichloromethane (80.0 mL) was added to the mixture dropwise. The mixture was stirred at 0° C. for 0.5 h. A solution of methyl 2-aminoacetate; hydrochloride (13.6 g, 0.109 mol) and triethylamine (11.0 g, 0.109 mol) in dichloromethane (80.0 mL) (that had been stirred together for 30 min at 20° C.) was added to the reaction mixture dropwise. The mixture was stirred at 0° C. for 1 h. The mixture was quenched by water (300 ml), extracted with EtOAc (200 mL×3). The combined organics were washed with brine (150 mL), dried over Na₂SO₄, filtered and concentrated under reduced pressure to give a residue. The residue was purified by flash column (silica, 10-15% ethyl acetate in petroleum ether) to give methyl 2-[[S-(3-bromo-2-fluoro-phenyl)-N-[tert-butyl(dimethyl)silyl]sulfonimidoyl]amino]acetate. 1H NMR (400 MHz, chloroform-d) δ=7.82 (t, J=7.2 Hz, 1H), 7.72-7.63 (m, 1H), 7.10 (t, J=7.9 Hz, 1H), 5.11 (br t, J=5.1 Hz, 1H), 3.93-3.74 (m, 2H), 3.64 (d, J=0.9 Hz, 3H), 0.92 (d, J=0.9 Hz, 10H), 0.10 (s, 6H)

Step 3. To a solution of methyl 2-[[S-(3-bromo-2-fluoro-phenyl)-N-[tert-butyl(dimethyl)silyl]sulfonimidoyl]amino]acetate (7.3 g, 0.0166 mol) in THF (70.0 mL) was added tetrabutylammonium fluoride (1.00 M in THF, 83.1 mL, 0.0831 mol). The mixture was allowed to 50° C. and stirred for 2 h. The mixture was cooled to 20° C. and quenched by saturated NH₄Cl aqueous solution (100 ml), extracted with ethyl acetate (100 mL×3). The combined organics were washed with brine (150 mL), dried over Na₂SO₄, filtered and concentrated under reduced pressure to give a residue. The residue was purified by flash column (silica, 5˜10% methanol in dichloromethane) to give 1-(3-bromo-2-fluorophenyl)-2H-1,2,5-thiadiazol-4(3H)-one 1-oxide. 1H NMR (400 MHz, DMSO-d6) δ=9.65 (br s, 1H), 8.21-8.12 (m, 1H), 8.03-7.92 (m, 1H), 7.45 (t, J=8.1 Hz, 1H), 4.19 (dd, J=1.9, 14.4 Hz, 1H), 4.06-3.98 (m, 1H)

Step 4. To a solution of 1-(3-bromo-2-fluorophenyl)-2H-1,2,5-thiadiazol-4(3H)-one 1-oxide (2.1 g, 0.00716 mol) and hypodiboric acid (6.4 g, 0.0716 mol) in methanol (30.0 mL) were added triethylamine (2.2 g, 0.0215 mol) and bis(1-adamantyl)-butyl-phosphane; methylsulfonyloxy-[2-(o-tolyl)phenyl]palladium(521 mg, 0.000716 mol). The reaction mixture was degassed, purged with N₂ for three times. The mixture was stirred at 25° C. for 30 min. for under N₂ atmosphere. The mixture was filtered and the filter cake was rinsed with MeOH (5 mL×3). Then the combined filtrates were concentrated under reduced pressure to give a residue. The residue was purified by prep-HPLC (column: Phenomenex luna C18 (250*70 mm, 15 um); mobile phase: [A: H2O (0.1% TFA); B: ACN]; B %: 1.00%-20.00%) to give 2-fluoro-3-(1-oxido-4-oxo-3,4-dihydro-2H-1λ⁶,2,5-thiadiazol-1-yl)phenyl)boronic acid. 1H NMR (400 MHz, DMSO-d6) δ=9.43 (s, 1H), 8.60 (s, 2H), 8.01-7.87 (m, 2H), 7.43 (t, J=7.6 Hz, 1H), 4.15 (dd, J=1.8, 14.3 Hz, 1H), 3.95 (d, J=14.3 Hz, 1H)

### Preparation of (2-fluoro-3-(1-oxido-4,5-dihydro-3H-1λ⁶-isothiazol-1-yl)phenyl)boronic acid (C20.2)

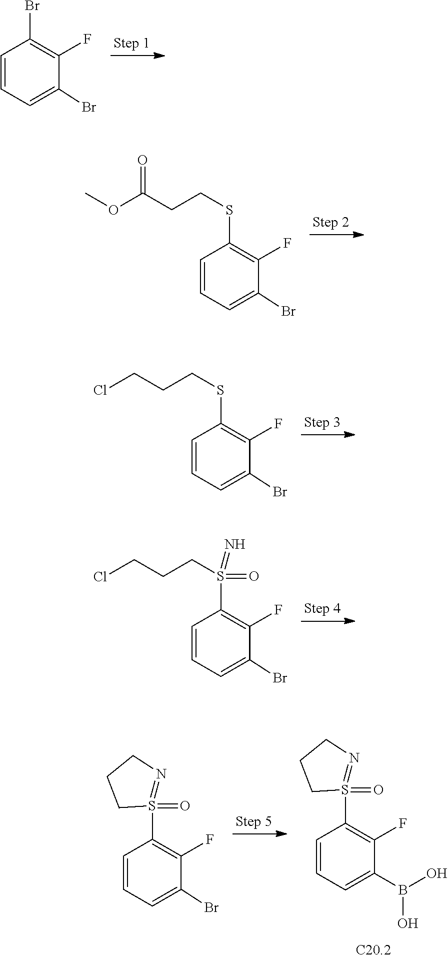 [SMILES: Fc1c(Br)cccc1Br ; COC(=O)CCSc1cccc(Br)c1F ; Fc1c(Br)cccc1SCCCCl ; N=S(=O)(CCCCl)c1cccc(Br)c1F ; O=S1(c2cccc(Br)c2F)=NCCC1 ; O=S1(c2cccc(B(O)O)c2F)=NCCC1]

Step 1. To a solution of 1,3-dibromo-2-fluorobenzene (5.0 g, 0.0197 mol) and methyl 3-sulfanylpropanoate (2.1 g, 0.0177 mol) in 1,4-dioxane (75 mL) was added tris(dibenzylideneacetone)dipalladium (797 mg, 0.000985 mol) and 4,5-bis(diphenylphosphino)-9,9-dimethylxanthene (1.1 g, 0.00197 mol). The reaction mixture was degassed and purged with N₂ for three times, heated to 100° C., stirred for 16 h under N₂ atmosphere. The mixture was quenched by water (100 ml), extracted with EtOAc (100 mL×3). The combined organics were washed with brine (100 mL), dried over Na₂SO₄, filtered and concentrated under reduced pressure to give a residue. The residue was purified by flash column (silica, 5-10% ethyl acetate in petroleum ether) to give methyl 3-((3-bromo-2-fluorophenyl)thio)propanoate. ¹H NMR (400 MHz, chloroform-d) δ=7.49-7.42 (m, 1H), 7.33 (t, J=7.2 Hz, 1H), 6.98 (t, J=7.9 Hz, 1H), 3.69 (s, 3H), 3.17 (t, J=7.4 Hz, 2H), 2.63 (t, J=7.3 Hz, 2H)

Step 2. To a solution of methyl 3-((3-bromo-2-fluorophenyl)thio)propanoate (3.2 g, 0.0109 mol) in ethanol (40 mL) was added sodium ethoxide (2.2 g, 0.0327 mol) in portions. The mixture was stirred at 25° C. for 1 h. Then 1-bromo-3-chloro-propane (3.4 g, 0.0218 mol) was added to the mixture dropwise. The mixture was stirred at 25° C. for 1 h. The mixture was quenched by water (50 ml), extracted with EtOAc (50 mL×3). The combined organics were washed with brine (50 mL), dried over Na₂SO₄, filtered and concentrated under reduced pressure to give a residue. The residue was purified by flash column (silica, 1-5% ethyl acetate in petroleum ether) to give (3-bromo-2-fluorophenyl)(3-chloropropyl)sulfane. ¹H NMR (400 MHz, chloroform-d) δ=7.44 (ddd, J=1.6, 6.4, 7.9 Hz, 1H), 7.33 (ddd, J=1.6, 6.6, 7.9 Hz, 1H), 6.99 (dt, J=0.8, 7.9 Hz, 1H), 3.68 (t, J=6.3 Hz, 2H), 3.09 (t, J=6.9 Hz, 2H), 2.06 (quin, J=6.6 Hz, 2H)

Step 3. To a solution of (3-bromo-2-fluorophenyl)(3-chloropropyl)sulfane (2.7 g, 0.00970 mol) and ammonium carbonate (1.4 g, 0.0146 mol) in methanol (50.0 mL) was added iodobenzene diacetate (6.6 g, 0.0204 mol) in portions. The mixture was stirred at 25° C. for 1 h. The mixture was concentrated under reduced pressure to give a residue. The residue was purified by flash column (silica, 20-25% ethyl acetate in petroleum ether) to give 1-bromo-3-(3-chloropropylsulfonimidoyl)-2-fluorobenzene. ¹H NMR (400 MHz, CHLOROFORM-d) δ=7.89 (ddd, J=1.6, 6.3, 7.9 Hz, 1H), 7.83 (ddd, J=1.7, 6.3, 7.9 Hz, 1H), 7.23 (dt, J=0.8, 7.9 Hz, 1H), 3.67 (t, J=6.3 Hz, 2H), 3.61-3.47 (m, 2H), 2.40-2.20 (m, 2H)

Step 4. To a solution of 1-bromo-3-(3-chloropropylsulfonimidoyl)-2-fluorobenzene (2.1 g, 0.00668 mol) in DMF (50.0 mL) was added potassium carbonate (2.8 g, 0.0200 mol). The mixture was stirred at 100° C. for 1 h. The mixture was cooled to 25° C. and quenched by ice water (80 mL), extracted with ethyl acetate (80 mL×3). The combined organics were washed with brine (50 mL), dried over Na₂SO₄, filtered and concentrated under reduced pressure to give a residue. The residue was purified by flash column (silica, 40-45% ethyl acetate in petroleum ether, gradient over 10 min) to give 1-(3-bromo-2-fluorophenyl)-4,5-dihydro-3H-isothiazole 1-oxide. ¹H NMR (400 MHz, chloroform-d) δ=8.04-7.96 (m, 1H), 7.80 (dt, J=1.1, 7.2 Hz, 1H), 7.21 (t, J=7.9 Hz, 1H), 4.14-4.06 (m, 1H), 3.70 (td, J=7.1, 10.6 Hz, 1H), 3.49 (t, J=7.8 Hz, 2H), 2.51-2.31 (m, 2H)

Step 5. To a solution of 1-(3-bromo-2-fluorophenyl)-4,5-dihydro-3H-isothiazole 1-oxide (1.3 g, 0.00467 mol) and hypodiboric acid (4.2 g, 0.0468 mol) in methanol (20.0 mL) were added triethylamine (1.4 g, 0.0140 mol) and bis(1-adamantyl)-butyl-phosphane; methylsulfonyloxy-[2-(o-tolyl)phenyl]palladium(340 mg, 0.000467 mol). The mixture was degassed, purged with N₂ for three times. The mixture was stirred at 25° C. for 1 h under N₂ atmosphere. The mixture was filtered and the filter cake was rinsed with MeOH (5 mL*3). Then the combined filtrates were concentrated under reduced pressure to give a residue. The residue was purified by prep-HPLC (column: Kromasil C18 (w) 250*50 mm 10 u; mobile phase: [A: H₂O (10 mM NH₄HCO₃); B: ACN]; B %: 5.00%-50.00%) to give (2-fluoro-3-(1-oxido-4,5-dihydro-3H-1×⁶-isothiazol-1-yl)phenyl)boronic acid. ¹HNMR (400 MHz, DMSO-d₆) δ=7.91-7.74 (m, 2H), 7.34 (br t, J=7.4 Hz, 1H), 3.82-3.75 (m, 1H), 3.58-3.49 (m, 1H), 3.47-3.38 (m, 2H), 2.22 (br d, J=6.3 Hz, 2H)

### Preparation of (7-fluoro-1-methyl-1-oxido-3H-1V-benzo[d]isothiazol-6-yl)boronic acid (C20.3)

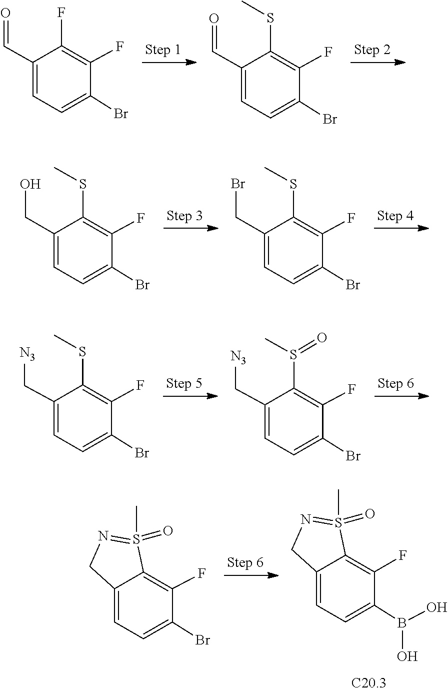

Step 1. To a solution of 4-bromo-2,3-difluorobenzaldehyde (9.0 g, 0.0407 mol) in N,N-dimethylformamide (100.0 mL) was added potassium carbonate (6.7 g, 0.0489 mol) and methanethiol (10.0%, 19.6 g, 0.0407 mol). The reaction mixture was degassed and purged with N₂ for 3 times, then stirred at 25° C. for 16 h under N₂ atmosphere. The reaction mixture was quenched by saturated ammonium chloride aqueous (100 mL), extracted with ethyl acetate (100 mL×2). The combined organics were washed with brine (100 mL), dried over Na₂SO₄, filtered and concentrated under reduced pressure to give a residue. The residue was purified by silica gel chromatography (silica, 0-5% ethyl acetate in petroleum ether) to afford 4-bromo-3-fluoro-2-(methylthio)benzaldehyde. ¹H NMR (400 MHz, chloroform-d) δ=7.22 (d, J=7.5 Hz, 1H), 6.62 (d, J=7.5 Hz, 1H), 4.90-4.24 (m, 2H), 2.10 (s, 3H)

Step 2. To a solution of 4-bromo-3-fluoro-2-(methylthio)benzaldehyde (4.2 g, 0.0137 mol) in methanol (40.0 mL) at 0° C., sodium borohydride (776.0 mg, 0.0205 mol) was added portionwise. The reaction mixture was stirred at 0° C. for 0.5 h. The reaction mixture was poured into 50 mL saturated ammonium chloride aqueous, and most of methyl alcohol was removed by evaporation and the resulting mixture was extracted ethyl acetate (50 mL×2). The combined organics were washed with brine (50 mL), dried over Na₂SO₄, filtered and concentrated under reduced pressure to give a residue. The residue was purified by silica gel chromatography (silica, 0-25% ethyl acetate in petroleum ether) to afford (4-bromo-3-fluoro-2-(methylthio)phenyl)methanol. ¹H NMR (400 MHz, chloroform-d) δ=7.50 (dd, J=6.8, 8.1 Hz, 1H), 7.14 (d, J=8.3 Hz, 1H), 4.82 (s, 2H), 2.47 (s, 3H)

Step 3. To a solution of (4-bromo-3-fluoro-2-(methylthio)phenyl)methanol (2.7 g, 0.0105 mol) in dichloromethane (30.0 mL) was added phosphorous tribromide (873.0 mg, 0.00316 mol) and the mixture was stirred at 20° C. for 16 h. The mixture was concentrated and the residue was purified by silica gel chromatography (ISCO 20 g silica, 0-5% ethyl acetate in petroleum ether) to afford (3-bromo-6-(bromomethyl)-2-fluorophenyl)(methyl)sulfane. ¹H NMR (400 MHz, chloroform-d) δ=7.48 (dd, J=6.8, 8.4 Hz, 1H), 7.13 (dd, J=1.3, 8.3 Hz, 1H), 4.72 (s, 2H), 2.50 (d, J=0.6 Hz, 3H)

Step 4. To a solution of (3-bromo-6-(bromomethyl)-2-fluorophenyl)(methyl)sulfane (500.0 mg, 0.00158 mol) in hexamethylphosphoramide (5.0 mL) was added azidotrimethylsilane (218.0 mg, 0.00189 mol). The reaction mixture was degassed and purged with N₂ for 3 times, then stirred at 65° C. for 16 h under N₂ atmosphere. The reaction mixture was cooled to room temperature and diluted with ice water (10 mL). Then the combined filtrates were extracted with ethyl acetate (10 mL×3). The aqueous phase was adjusted pH >9 by added saturated NaOH aqueous solution slowly at 0° C., then added sodium hypochlorite solution and soak overnight. The combined organics were washed with water (10 ml×3), brine (10 mL), dried over Na₂SO₄, filtered and concentrated under reduced pressure to give (6-(azidomethyl)-3-bromo-2-fluorophenyl)(methyl)sulfane. ¹H NMR (400 MHz, chloroform-d) δ=7.51 (dd, J=6.8, 8.2 Hz, 1H), 7.07 (d, J=8.1 Hz, 1H), 4.56 (s, 2H), 2.45 (s, 3H)

Step 5. To a solution of (6-(azidomethyl)-3-bromo-2-fluorophenyl)(methyl)sulfane (1.2 g, 0.00395 mol) in dichloromethane (15.0 mL) was cooled to −30° C., then was added 3-chloroperoxybenzoic acid (0.9 g, 0.00443 mol). The reaction mixture was degassed and purged with N₂ for 3 times, then stirred at −30° C. for 1 h under N₂ atmosphere. The reaction mixture was cooled to room temperature and quenched by saturated sodium sulfite solution (20 mL). Then the combined filtrates were extracted with ethyl acetate (15 mL×3). The combined organics were washed with NaHCO₃ (15 ml×3), brine (15 mL), dried over Na₂SO₄, filtered and concentrated under reduced pressure to give 1-(azidomethyl)-4-bromo-3-fluoro-2-(methylsulfinyl)benzene. ¹H NMR (400 MHz, CHLOROFORM-d) δ=7.70 (dd, J=6.8, 8.3 Hz, 1H), 7.14 (d, J=8.3 Hz, 1H), 4.90-4.85 (m, 1H), 4.76-4.71 (m, 1H), 3.09 (s, 3H).

Step 6. To a solution of 1-(azidomethyl)-4-bromo-3-fluoro-2-(methylsulfinyl)benzene (1.1 g, 0.00369 mol) in PhMe (12.0 mL) was added iron(II) phthalocyanine (0.5 g, 0.000948 mol). The reaction mixture was degassed and purged with N₂ for 3 times, then stirred at 110° C. for 16 h under N₂ atmosphere. The residue was purified by silica gel chromatography (silica, 0-60% ethyl acetate in petroleum ether) to afford 6-bromo-7-fluoro-1-methyl-3H-114-benzo[d]isothiazole 1-oxide. ¹H NMR (400 MHz, chloroform-d) δ=7.78 (dd, J=6.0, 8.0 Hz, 1H), 7.19 (d, J=8.1 Hz, 1H), 4.94 (br d, J=17.4 Hz, 1H), 4.73 (br d, J=17.6 Hz, 1H), 3.53 (s, 3H).

Step 7. To a solution of 6-bromo-7-fluoro-1-methyl-3H-114-benzo[d]isothiazole 1-oxide (400 mg, 0.00147 mol) in 1,4-dioxane (4.0 mL) was added 4,4,5,5-tetramethyl-2-(4,4,5,5-tetramethyl-1,3,2-dioxaborolan-2-yl)-1,3,2-dioxaborolane (933.0 mg, 0.00367 mol), Pd(dppf)Cl₂ (89.2 mg, 0.000148 mol) and potassium acetate (432.0 mg, 0.00440 mol). The reaction mixture was degassed and purged with N₂ for 3 times, then stirred at 80° C. for 16 h under N₂ atmosphere. The resultant mixture was filtered through a syringe filter and the combined filtrates were concentrated. The residue was purified by prep-HPLC (column: Phenomenex Luna C1875*30 mm*3 um; mobile phase: [A: water (0.04% HCl); B: ACN]; B %: 1.00%-12.00%) to give (7-fluoro-1-methyl-1-oxido-3H-1λ⁴-benzo[d]isothiazol-6-yl)boronic acid. ¹H NMR (400 MHz, DMSO-d6) δ=8.05 (dd, J=5.4, 7.6 Hz, 1H), 7.53 (d, J=7.6 Hz, 1H), 5.03-4.91 (m, 1H), 4.80 (br d, J=16.7 Hz, 1H), 4.07-3.78 (m, 3H)

### Preparation of 3-chloro-2′-(3-(dimethylcarbamoyl)-2-fluorophenyl)-5′,6-dimethyl-2-oxo-2H-[1,4′-bipyridin]-4-yl trifluoromethanesulfonate (D1.1a)

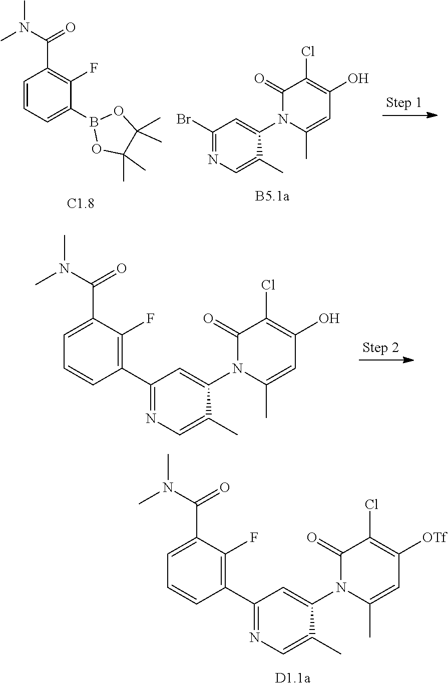

Step 1. A vial was charged with 2′-bromo-3-chloro-4-hydroxy-5′,6-dimethyl-2H-[1,4′-bipyridin]-2-one (B5.1a) (200 mg, 0.61 mmol), 2-fluoro-N,N-dimethyl-3-(4,4,5,5-tetramethyl-1,3,2-dioxaborolan-2-yl)benzamide (C1.8) (213 mg, 0.73 mmol), SPhos Pd G4 (48 mg, 0.067 mmol), Na₂CO₃ (2M aq., 1.2 mL), and 1,4-dioxane (6 mL). The mixture was sparged with argon for 2 min., and then heated to 80° C. for 1 hr. The mixture was filtered over celite, washing with MeOH. The filtrate was concentrated and dried overnight under vacuum. The residue was used directly in the next step.

Step 2. The crude residue from Step 1 was dissolved in DCM (10 mL), followed by addition of Et₃N (0.25 mL, 0.18 mmol). The mixture was cooled to 0° C. and Tf₂O (0.12 mL, 0.73 mmol) was added dropwise. The mixture was allowed to stir for 1 hr., followed by addition of water (20 mL). The mixture was extracted with DCM (3×10 mL). The combined organics were dried over Na₂SO₄, filtered, and concentrated. The residue was subjected to flash column chromatography (SiO₂, hexanes-EtOAc) to provide 3-chloro-2′-(3-(dimethylcarbamoyl)-2-fluorophenyl)-5′,6-dimethyl-2-oxo-2H-[1,4′-bipyridin]-4-yl trifluoromethanesulfonate (D1.1a). ES/MS m/z: 548.1 [M+H]+

(rac)-3-chloro-2′-(3-(dimethylcarbamoyl)-2-fluorophenyl)-5′,6-dimethyl-2-oxo-2H-[1,4′-bipyridin]-4-yl trifluoromethanesulfonate (D1.1) D1.1 was prepared analogously to D1.1a starting with B5.1.

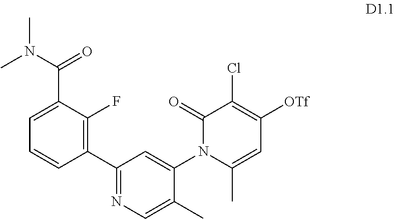 [SMILES: Cc1cnc(-c2cccc(C(=O)N(C)C)c2F)cc1-n1c(C)cc(OS(=O)(=O)C(F)(F)F)c(Cl)c1=O]

3,5′-dichloro-2′-(3-(dimethylcarbamoyl)-2-fluorophenyl)-6-methyl-2-oxo-2H-[1,4′-bipyridin]-4-yl trifluoromethanesulfonate (D1.2) D1.2 was prepared analogously to D1.1a starting with B2.4, using C1.9 instead of C1.8 and PhN(Tf)₂ instead of Tf₂O in Step 2.

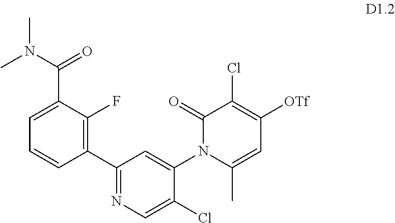 [SMILES: Cc1cc(OS(=O)(=O)C(F)(F)F)c(Cl)c(=O)n1-c1cc(-c2cccc(C(=O)N(C)C)c2F)ncc1Cl]

3,3′-dichloro-2′-(3-(dimethylcarbamoyl)-2-fluorophenyl)-6-methyl-2-oxo-2H-[1,4′-bipyridin]-4-yl trifluoromethanesulfonate (D1.3) D1.3 was prepared analogously to D1.1a starting with B2.5, using C1.9 instead of C1.8 and PhN(Tf)₂ instead of Tf₂O in Step 2.

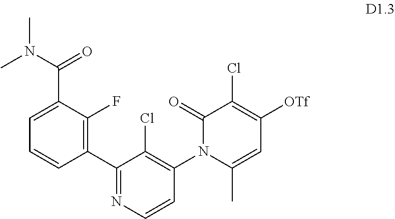 [SMILES: Cc1cc(OS(=O)(=O)C(F)(F)F)c(Cl)c(=O)n1-c1ccnc(-c2cccc(C(=O)N(C)C)c2F)c1Cl]

3,5′-dichloro-2′-(2-fluoro-3-(methylcarbamoyl)phenyl)-6-methyl-2-oxo-2H-[1,4′-bipyridin]-4-yl trifluoromethanesulfonate (D1.4) D1.4 was prepared analogously to D1.1a starting with B2.4, using C1.13 instead of C1.8 and PhN(Tf)₂ instead of Tf₂O in Step 2.

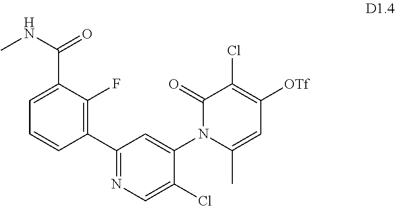 [SMILES: CNC(=O)c1cccc(-c2cc(-n3c(C)cc(OS(=O)(=O)C(F)(F)F)c(Cl)c3=O)c(Cl)cn2)c1F]

3-chloro-2′-(2-fluoro-3-(1-methylcyclopropane-1-carboxamido)phenyl)-5′,6-dimethyl-2-oxo-2H-[1,4′-bipyridin]-4-yl trifluoromethanesulfonate (D1.5) D1.5 was prepared analogously to D1.1a using C7.10 instead of C1.8.

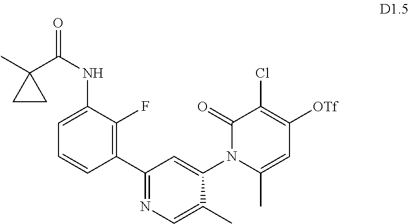

3-chloro-3′-fluoro-2′-(2-fluoro-3-(1-methylcyclopropane-1-carboxamido)phenyl)-5′,6-dimethyl-2-oxo-2H-[1,4′-bipyridin]-4-yl trifluoromethanesulfonate (D1.6) D1.6 was prepared analogously to D1.1a using C7.10 instead of C1.8 and B3.2a instead of B5.1a.

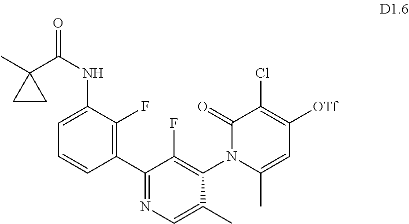

3-chloro-2′-(3-(cyclopropylcarbamoyl)-2-fluorophenyl)-5′,6-dimethyl-2-oxo-2H-[1,4′-bipyridin]-4-yl trifluoromethanesulfonate (D1.7) D1.7 was prepared analogously to D1.1a using C1.21 instead of C1.8.

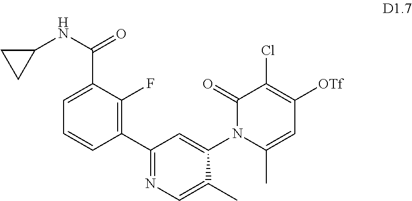

3-chloro-2′-(3-(dimethylcarbamoyl)-2-fluorophenyl)-3′-fluoro-5′,6-dimethyl-2-oxo-2H-[1,4′-bipyridin]-4-yl trifluoromethanesulfonate (D1.8) D1.8 was prepared analogously to D1.1a using C7.10 instead of C1.8 and B3.2a instead of B5.1a.

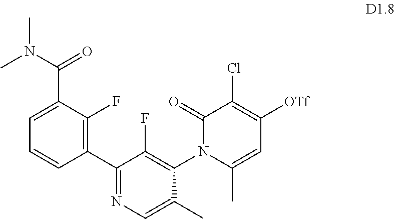

3-chloro-2′-(3-(1-cyclopropyl-1H-1,2,4-triazol-5-yl)-2-fluorophenyl)-3′-fluoro-5′,6-dimethyl-2-oxo-2H-[1,4′-bipyridin]-4-yl trifluoromethanesulfonate (D1.9). D1.9 was prepared analogously to D1.1a starting from B2.2a instead of B5.1a and C12.4 instead of C1.8, using PdCl₂(dppf)-DCM instead of SPhos Pd G4 in Step 1.

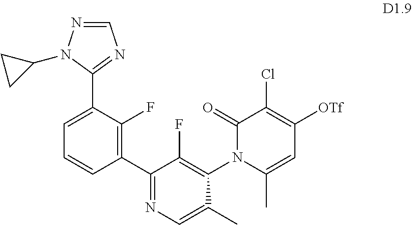

3-chloro-2′-(3-(1-(difluoromethyl)-1H-1,2,4-triazol-5-yl)-2-fluorophenyl)-3′-fluoro-5′,6-dimethyl-2-oxo-2H-[1,4′-bipyridin]-4-yl trifluoromethanesulfonate (D1.10). D1.10 was prepared analogously to D1.1a starting from B2.2a instead of B5.1a and C15.1 instead of C1.8, using PdCl₂(dppf)-DCM instead of SPhos Pd G4 in Step 1.

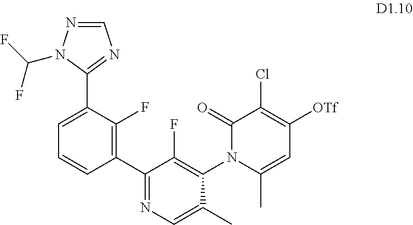

3-chloro-2′-(3-(3,5-dimethyl-1H-1,2,4-triazol-1-yl)-2-fluorophenyl)-3′-fluoro-5′,6-dimethyl-2-oxo-2H-[1,4′-bipyridin]-4-yl trifluoromethanesulfonate (D1.11). D1.11 was prepared analogously to D1.1a starting from B2.2a instead of B5.1a and C13.3 instead of C1.8. Using PdCl₂(dppf)-DCM instead of SPhos Pd G4 and Cs₂CO₃ instead of Na₂CO₃ in Step 1, and PhN(Tf)₂ instead of Tf₂O in Step 2.

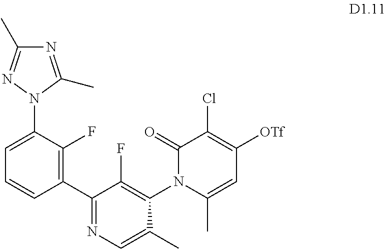

3-chloro-2′-(3-(3,5-dimethyl-1H-pyrazol-1-yl)-2-fluorophenyl)-3′-fluoro-5′,6-dimethyl-2-oxo-2H-[1,4′-bipyridin]-4-yl trifluoromethanesulfonate (D1.12). D1.12 was prepared analogously to D1.1a starting from B2.2a instead of B5.1a and C13.1 instead of C1.8. Using PdCl₂(dppf) instead of SPhos Pd G4 and Cs₂CO₃ instead of Na₂CO₃ in Step 1, and PhN(Tf)₂ instead of Tf₂O in Step 2.

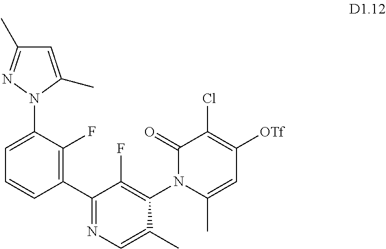

3-chloro-3′-fluoro-2′-(2-fluoro-3-(3,4,5-trimethyl-1H-pyrazol-1-yl)phenyl)-5′,6-dimethyl-2-oxo-2H-[1,4′-bipyridin]-4-yl trifluoromethanesulfonate (D1.13). D1.13 was prepared analogously to D1.1a starting from B2.2a instead of B5.1a and C13.2 instead of C1.8. Using PdCl₂(dppf) instead of SPhos Pd G4 and Cs₂CO₃ instead of Na₂CO₃ in Step 1, and PhN(Tf)₂ instead of Tf₂O in Step 2.

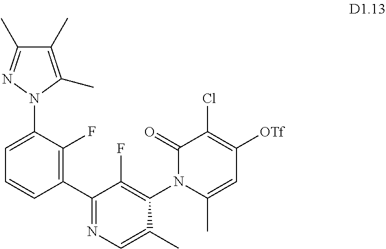

2′-(3-(2-acetamidopropan-2-yl)-2-fluorophenyl)-3-chloro-3′-fluoro-5′,6-dimethyl-2-oxo-2H-[1,4′-bipyridin]-4-yl trifluoromethanesulfonate (D1.14). D1.14 was prepared analogously to D1.1a starting from B2.2a instead of B5.1a and C17.1 instead of C1.8. Using PdCl₂(dppf) instead of SPhos Pd G4 and Cs₂CO₃ instead of Na₂CO₃ in Step 1.

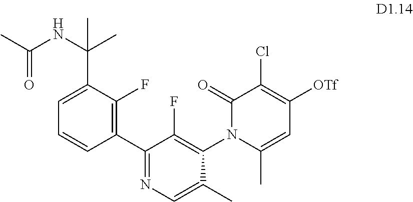

3,5′-dichloro-3′-fluoro-2′-(2-fluoro-3-(methylsulfonyl)phenyl)-6-methyl-2-oxo-2H-[1,4′-bipyridin]-4-yl trifluoromethanesulfonate (D1.15). D1.15 was prepared analogously to D1.1a starting from B2.6a instead of B5.1a and C17.1 instead of C1.8, using PdCl₂(dppf) instead of SPhos Pd G4 and Cs₂CO₃ instead of Na₂CO₃ in Step 1, and using PhN(OTf)₂ instead of Tf₂O in step 2.

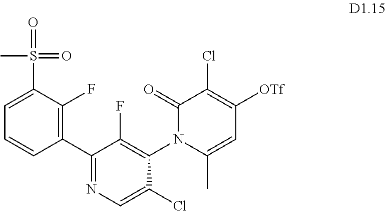

3,3′-dichloro-2′-(2-fluoro-3-(methylsulfonyl)phenyl)-5′,6-dimethyl-2-oxo-2H-[1,4′-bipyridin]-4-yl trifluoromethanesulfonate (D1.16). D1.16 was prepared analogously to D1.1a starting from B2.7a instead of B5.1a and C17.1 instead of C1.8, using PdCl₂(dppf) instead of SPhos Pd G4 and Cs₂CO₃ instead of Na₂CO₃ in Step 1, and using PhN(OTf)₂ instead of Tf₂O in step 2.

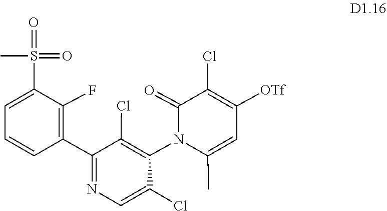

### Preparation of (rac)-3-(3-chloro-4-iodo-5′,6-dimethyl-2-oxo-2H-[1,4′-bipyridin]-2′-yl)-2-fluoro-N,N-dimethylbenzamide (D2.1)

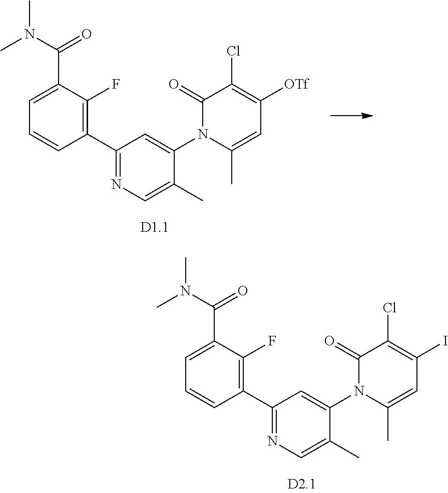 [SMILES: Cc1cnc(-c2cccc(C(=O)N(C)C)c2F)cc1-n1c(C)cc(OS(=O)(=O)C(F)(F)F)c(Cl)c1=O ; Cc1cnc(-c2cccc(C(=O)N(C)C)c2F)cc1-n1c(C)cc(I)c(Cl)c1=O]

A vial was charged with (rac)-3-chloro-2′-(3-(dimethylcarbamoyl)-2-fluorophenyl)-5′,6-dimethyl-2-oxo-2H-[1,4′-bipyridin]-4-yl trifluoromethanesulfonate (D1.1) (444 mg, 0.856 mmol), NaI (3.85 g, 25.7 mmol) and acetone (5 mL). The mixture was heated to 70° C. for 3 days. The mixture was diluted with DCM (20 mL), filtered, and concentrated. The residue was subjected to flash column chromatography (SiO₂, hexanes-EtOAc) to provide (rac)-3-(3-chloro-4-iodo-5′,6-dimethyl-2-oxo-2H-[1,4′-bipyridin]-2′-yl)-2-fluoro-N,N-dimethylbenzamide (D2.1).

### Preparation of N-(3-(3-chloro-5′,6-dimethyl-2-oxo-4-((1S,2S)-2-(4,4,5,5-tetramethyl-1,3,2-dioxaborolan-2-yl)cyclopropyl)-2H-[1,4′-bipyridin]-2′-yl)-2-fluorophenyl)-1-methylcyclopropane-1-carboxamide (D3.1)

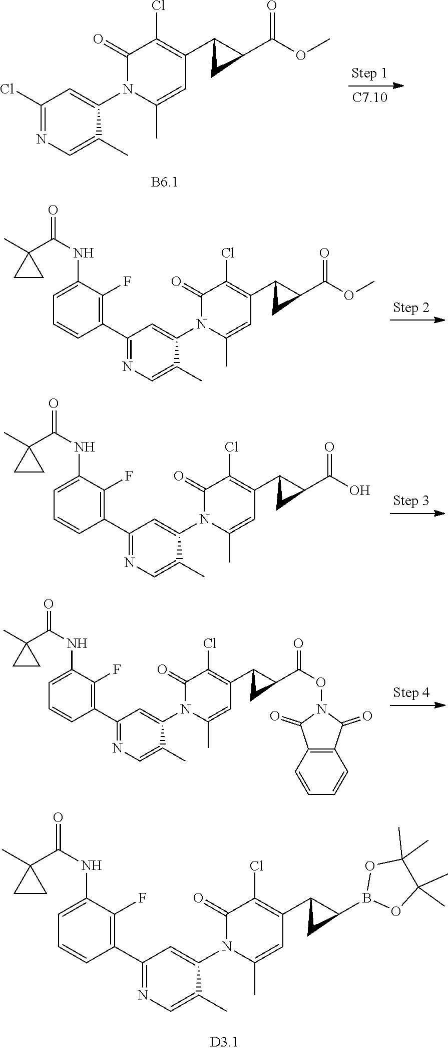

Step 1. A vial was charged with methyl (1S,2S)-2-(2′,3-dichloro-5′,6-dimethyl-2-oxo-2H-[1,4′-bipyridin]-4-yl)cyclopropane-1-carboxylate (B6.1) (1.00 g, 2.72 mmol), N-(2-fluoro-3-(4,4,5,5-tetramethyl-1,3,2-dioxaborolan-2-yl)phenyl)-1-methylcyclopropane-1-carboxamide (C7.10) (0.96 g, 3.00 mmol), Pd(dppf)Cl₂-DCM (225 mg, 0.27 mmol), 1,4-dioxane (20 mL), and Na₂CO₃ (2M aq., 5.5 mL). The mixture was heated to 85° C. for 1 hr. The mixture was cooled to room temperature, filtered, and concentrated. The residue was subjected to flash column chromatography (SiO₂, hexanes-EtOAc) to give methyl (1S,2S)-2-(3-chloro-2′-(2-fluoro-3-(1-methylcyclopropane-1-carboxamido)phenyl)-5′,6-dimethyl-2-oxo-2H-[1,4′-bipyridin]-4-yl)cyclopropane-1-carboxylate.

Step 2. A vial was charged with (1S,2S)-2-(3-chloro-2′-(2-fluoro-3-(1-methylcyclopropane-1-carboxamido)phenyl)-5′,6-dimethyl-2-oxo-2H-[1,4′-bipyridin]-4-yl)cyclopropane-1-carboxylate (1.28 g, 2.45 mmol), LiOH (1M aq., 4.9 mL), THF (10 mL), and MeOH (10 mL). The mixture was stirred at 50° C. for 1 hr. After cooling to room temperature HCl (1M aq., 5 mL) was added and the mixture was extracted with EtOAc (5×10 mL). The combined organics were dried over Na₂SO₄, filtered, and concentrated to give crude (1S,2S)-2-(3-chloro-2′-(2-fluoro-3-(1-methylcyclopropane-1-carboxamido)phenyl)-5′,6-dimethyl-2-oxo-2H-[1,4′-bipyridin]-4-yl)cyclopropane-1-carboxylic acid which was used without further purification. ES/MS m/z: 510.3 [M+H]+.

Step 3. To crude (1S,2S)-2-(3-chloro-2′-(2-fluoro-3-(1-methylcyclopropane-1-carboxamido)phenyl)-5′,6-dimethyl-2-oxo-2H-[1,4′-bipyridin]-4-yl)cyclopropane-1-carboxylic acid was added N-hydroxyphthalimide (440 mg, 2.70 mmol), DMAP (30 mg, 0.25 mmol), and DCM (30 mL).

DIC (340 mg, 2.70 mmol) was added and the mixture stirred at room temperature for 30 min. The mixture was concentrated and the residue subjected to flash column chromatography (SiO₂, hexanes-EtOAc) to give 1,3-dioxoisoindolin-2-yl (1S,2S)-2-(3-chloro-2′-(2-fluoro-3-(1-methylcyclopropane-1-carboxamido)phenyl)-5′,6-dimethyl-2-oxo-2H-[1,4′-bipyridin]-4-yl)cyclopropane-1-carboxylate. ES/MS m/z: 655.2 [M+H]+

Step 4. A vial was charged with 1,3-dioxoisoindolin-2-yl (1S,2S)-2-(3-chloro-2′-(2-fluoro-3-(1-methylcyclopropane-1-carboxamido)phenyl)-5′,6-dimethyl-2-oxo-2H-[1,4′-bipyridin]-4-yl)cyclopropane-1-carboxylate (1.38 g, 2.10 mmol), B₂pin₂ (1.07 g, 4.20 mmol), and PhCF₃ (15 mL). The mixture was sparged with N₂ for 5 min., after which tert-butyl isonicotinate (188 mg, 1.05 mmol) was added. The mixture was heated to 40° C. for 18 hrs. The mixture was cooled to room temperature, concentrated, and subjected to flash column chromatography (SiO₂, hexanes-EtOAc) to give N-(3-(3-chloro-5′,6-dimethyl-2-oxo-4-((1S,2S)-2-(4,4,5,5-tetramethyl-1,3,2-dioxaborolan-2-yl)cyclopropyl)-2H-[1,4′-bipyridin]-2′-yl)-2-fluorophenyl)-1-methylcyclopropane-1-carboxamide (D3.1).

3-(3-chloro-5′,6-dimethyl-2-oxo-4-((1S,2S)-2-(4,4,5,5-tetramethyl-1,3,2-dioxaborolan-2-yl)cyclopropyl)-2H-[1,4′-bipyridin]-2′-yl)-2-fluoro-N,N-dimethylbenzamide (D3.2). D3.2 was prepared analogously to D3.1, using C1.8 instead of C7.10.

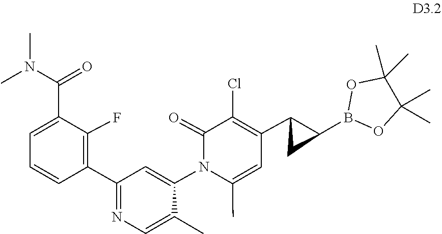

3-chloro-2′-(2-fluoro-3-(methylsulfonyl)phenyl)-5′,6-dimethyl-4-((1S,2S)-2-(4,4,5,5-tetramethyl-1,3,2-dioxaborolan-2-yl)cyclopropyl)-2H-[1,4′-bipyridin]-2-one (D3.3). D3.3 was prepared analogously to D3.1, using C1.17 instead of C7.10.

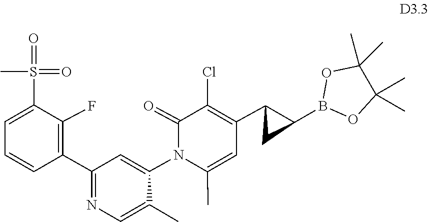

3-chloro-3′-fluoro-2′-(2-fluoro-3-(methylsulfonyl)phenyl)-5′,6-dimethyl-4-((1S,2S)-2-(4,4,5,5-tetramethyl-1,3,2-dioxaborolan-2-yl)cyclopropyl)-2H-[1,4′-bipyridin]-2-one (D3.4). D3.4 was prepared analogously to D3.1, using C1.17 instead of C7.10 and using B6.2 instead of B6.1.

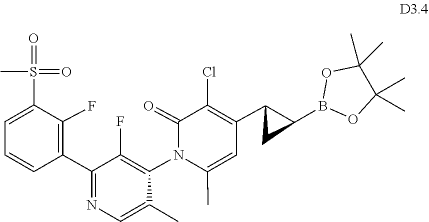

tert-butyl ((S)-(3-(3-chloro-3′-fluoro-5′,6-dimethyl-2-oxo-4-((1S,2S)-2-(4,4,5,5-tetramethyl-1,3,2-dioxaborolan-2-yl)cyclopropyl)-2H-[1,4′-bipyridin]-2′-yl)-2-fluorophenyl)(methyl)(oxo)-λ⁶-sulfaneylidene)carbamate (D3.5a) D3.5a was prepared analogously to D3.1, using C10.1a instead of C7.10 and using B6.2 instead of B6.1.

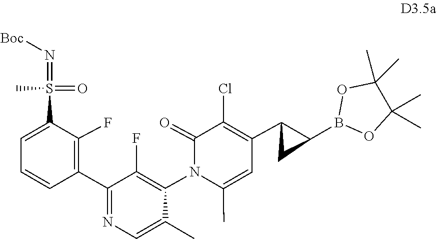

3-chloro-2′-(3-(1-(difluoromethyl)-1H-1,2,4-triazol-5-yl)-2-fluorophenyl)-3′-fluoro-5′,6-dimethyl-4-((1S,2S)-2-(4,4,5,5-tetramethyl-1,3,2-dioxaborolan-2-yl)cyclopropyl)-2H-[1,4′-bipyridin]-2-one (D3.6). D3.6 was prepared analogously to D3.1, using C15.1 instead of C7.10 and using B6.2 instead of B6.1, step 1 was heated to 100° C. instead of 85° C., and EtOAc was used instead of PhCF₃ for step 4.

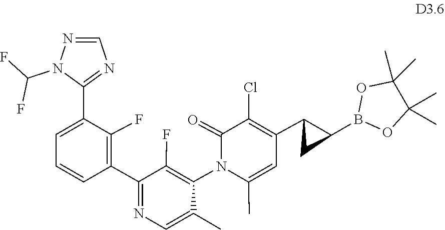

3-(3-chloro-3′-fluoro-5′,6-dimethyl-2-oxo-4-((1S,2S)-2-(4,4,5,5-tetramethyl-1,3,2-dioxaborolan-2-yl)cyclopropyl)-2H-[1,4′-bipyridin]-2′-yl)-N-cyclopropyl-2-fluorobenzamide (D3.7). D3.7 was prepared analogously to D3.1, using C1.21 instead of C7.10 and using B6.2 instead of B6.1 and EtOAc was used instead of PhCF₃ for step 4.

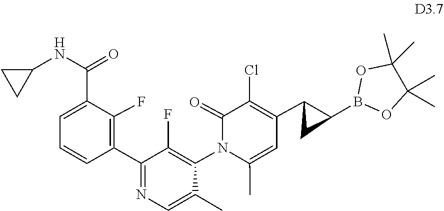

3-chloro-2″-(3,5-dimethyl-1H-1,2,4-triazol-1-yl)-3′,3″-difluoro-5′,6-dimethyl-4-((1S,2S)-2-(4,4,5,5-tetramethyl-1,3,2-dioxaborolan-2-yl)cyclopropyl)-2H-[1,4′:2′,4″-terpyridin]-2-one (D3.8). D3.8 was prepared analogously to D3.1, using C7.22 instead of C7.10 and using B6.2 instead of B6.1.

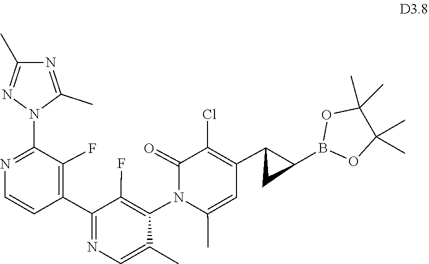

3-chloro-2′-(3-((dimethyl(oxo)-λ⁶-sulfaneylidene)amino)-2-fluorophenyl)-3′-fluoro-5′,6-dimethyl-4-((1S,2S)-2-(4,4,5,5-tetramethyl-1,3,2-dioxaborolan-2-yl)cyclopropyl)-2H-[1,4′-bipyridin]-2-one (D3.9). D3.9 was prepared analogously to D3.1, using C18.4 instead of C7.10 and using B6.2 instead of B6.1 and EtOAc was used instead of PhCF₃ for step 4.

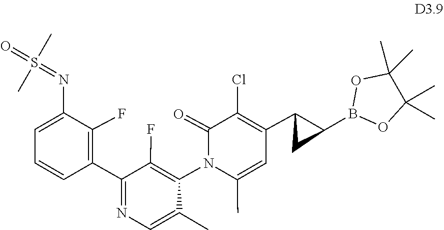

3-chloro-3′,3″-difluoro-5′,6-dimethyl-2″-(3-methyl-5-(trifluoromethyl)-1H-1,2,4-triazol-1-yl)-4-((1S,2S)-2-(4,4,5,5-tetramethyl-1,3,2-dioxaborolan-2-yl)cyclopropyl)-2H-[1,4′:2′,4″-terpyridin]-2-one (D3.10). D3.10 was prepared analogously to D3.1, using C7.24 instead of C7.10 and using B6.2 instead of B6.1 and EtOAc was used instead of PhCF₃ for step 4.

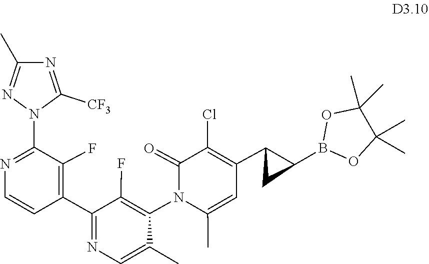

3-chloro-2′-(3-(3,5-dimethyl-1H-1,2,4-triazol-1-yl)-2-fluorophenyl)-3′-fluoro-5′,6-dimethyl-4-((1S,2S)-2-(4,4,5,5-tetramethyl-1,3,2-dioxaborolan-2-yl)cyclopropyl)-2H-[1,4′-bipyridin]-2-one (D3.11). D3.11 was prepared analogously to D3.1, using C13.3 instead of C7.10 and using B6.2 instead of B6.1 and EtOAc was used instead of PhCF₃ for step 4.

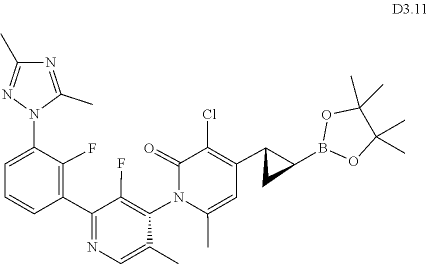

3-chloro-3′-fluoro-2′-(2-fluoro-3-(1-methyl-1H-1,2,4-triazol-5-yl)phenyl)-5′,6-dimethyl-4-((1S,2S)-2-(4,4,5,5-tetramethyl-1,3,2-dioxaborolan-2-yl)cyclopropyl)-2H-[1,4′-bipyridin]-2-one (D3.12). D3.12 was prepared analogously to D3.1, using C9.3 instead of C7.10 and using B6.2 instead of B6.1 and EtOAc was used instead of PhCF₃ for step 4.

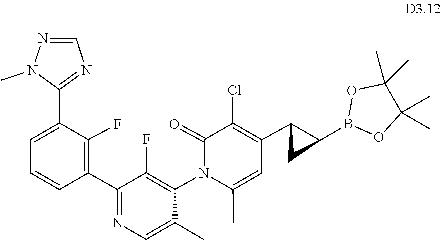

3-chloro-3′-fluoro-2′-(2-fluoro-3-((4-oxido-1,4λ⁶-oxathian-4-ylidene)amino)phenyl)-5′,6-dimethyl-4-((1S,2S)-2-(4,4,5,5-tetramethyl-1,3,2-dioxaborolan-2-yl)cyclopropyl)-2H-[1,4′-bipyridin]-2-one (D3.13). D3.13 was prepared analogously to D3.1, using C18.1 instead of C7.10 and using B6.2 instead of B6.1 and EtOAc was used instead of PhCF₃ for step 4.

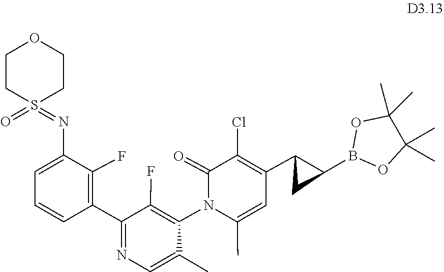

### Preparation of 2′,3-dichloro-4-((1S,2S)-2-(4-fluorophenyl)cyclopropyl)-5′,6-dimethyl-2H-[1,4′-bipyridin]-2-one (D4.1)

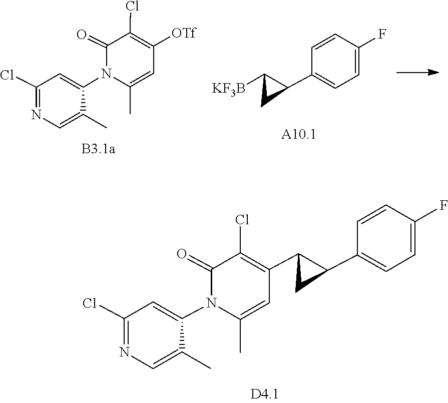

A vial was charged with 2′,3-dichloro-5′,6-dimethyl-2-oxo-2H-[1,4′-bipyridin]-4-yl trifluoromethanesulfonate (B3.1a) (800 mg, 1.92 mmol), trifluoro((1S,2S)-2-(4-fluorophenyl)cyclopropyl)-λ⁴-borane, potassium salt (A10.1) (696 mg, 2.88 mmol), Cs₂CO₃ (1.87 g, 5.75 mmol), Pd(dppf)Cl₂-DCM (157 mg, 0.19 mmol), PhMe (10 mL), and water (1 mL). The mixture was sparged with nitrogen for 2 minutes and was then heated to 80° C. for 30 min. The mixture was cooled to room temperature, filtered, concentrated, and subjected to flash column chromatography (SiO₂, hexanes-EtOAc-MeOH) to provide 2′,3-dichloro-4-((1S,2S)-2-(4-fluorophenyl)cyclopropyl)-5′,6-dimethyl-2H-[1,4′-bipyridin]-2-one (4.1).

2′,3-dichloro-3′-fluoro-4-((1S,2S)-2-(4-fluorophenyl)cyclopropyl)-5′,6-dimethyl-2H-[1,4′-bipyridin]-2-one (D4.2) D4.2 was prepared analogously to D4.1, using B3.2a instead of B3.1a.

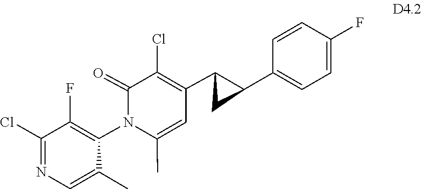

2′,3-dichloro-4-((1S,2S)-2-(1-(difluoromethyl)-1H-pyrazol-4-yl)cyclopropyl)-5′,6-dimethyl-2H-[1,4′-bipyridin]-2-one (D4.3) D4.3 was prepared analogously to D4.1, using A9.7 instead of A10.1.

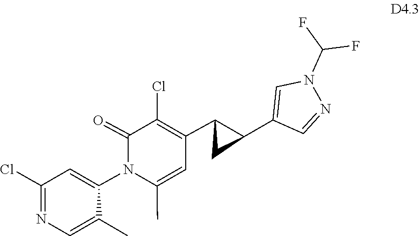

2′,3-dichloro-4-((1S,2S)-2-(1-(difluoromethyl)-1H-pyrazol-4-yl)cyclopropyl)-3′-fluoro-5′,6-dimethyl-2H-[1,4′-bipyridin]-2-one (D4.4) D4.4 was prepared analogously to D4.1, using B3.2a instead of B3.1a and A9.7 instead of A10.1.

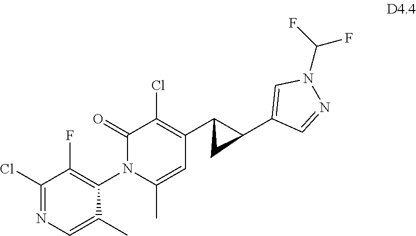

2′,3-dichloro-4-((1S,2S)-2-(5-chloropyridin-3-yl)cyclopropyl)-5′,6-dimethyl-2H-[1,4′-bipyridin]-2-one (D4.5) D4.5 was prepared analogously to D4.1, using A10.34 instead of A10.1.

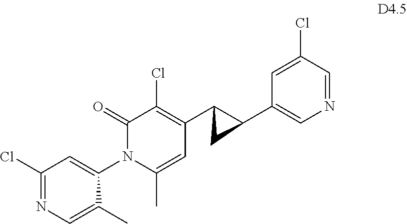

2′,3-dichloro-4-((1S,2S)-2-(5-chloropyridin-3-yl)cyclopropyl)-3′-fluoro-5′,6-dimethyl-2H-[1,4′-bipyridin]-2-one (D4.6) D4.6 was prepared analogously to D4.1, using B3.2a instead of B3.1a and A10.34 instead of A10.1.

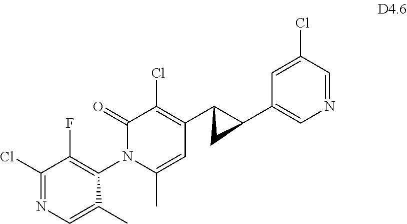

2′,3-dichloro-4-((1S,2S)-2-(5-fluoropyridin-3-yl)cyclopropyl)-5′,6-dimethyl-2H-[1,4′-bipyridin]-2-one (D4.7)) D4.7 was prepared analogously to D4.1, using A10.32 instead of A10.1.

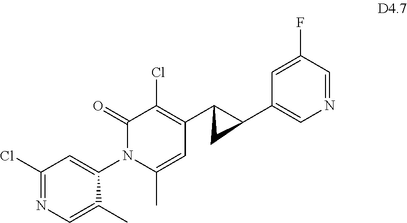

2′,3-dichloro-3′-fluoro-4-((1S,2S)-2-(5-fluoropyridin-3-yl)cyclopropyl)-5′,6-dimethyl-2H-[1,4′-bipyridin]-2-one (D4.8) D4.8 was prepared analogously to D4.1, using B3.2a instead of B3.1a and A10.32 instead of A10.1.

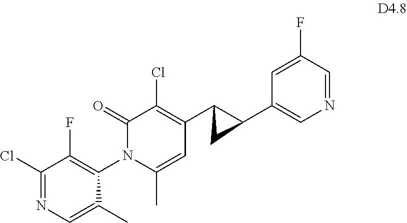

2′,3-dichloro-4-((1S,2S)-2-(5-(difluoromethyl)pyridin-3-yl)cyclopropyl)-3′-fluoro-5′,6-dimethyl-2H-[1,4′-bipyridin]-2-one (D4.9) D4.9 was prepared analogously to D4.1, using B3.2a instead of B3.1a and A9.6 instead of A10.1.

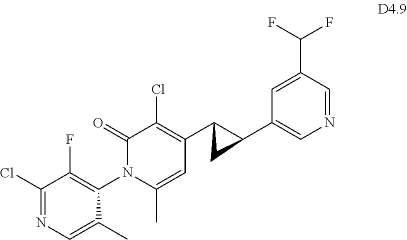

2′-bromo-3-chloro-3′-fluoro-4-((1S,2S)-2-(5-fluoropyridin-3-yl)cyclopropyl)-5′,6-dimethyl-2H-[1,4′-bipyridin]-2-one (D4.10). D4.10 was prepared analogously to D4.1, using B3.3a instead of B3.1a, A10.32 instead of A10.1.

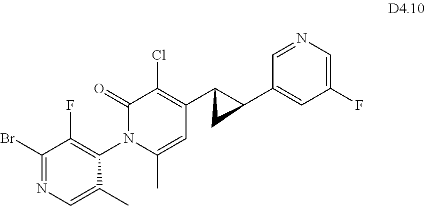

2′-bromo-3-chloro-4-((1S,2S)-2-(1-(difluoromethyl)-1H-pyrazol-4-yl)cyclopropyl)-3′-fluoro-5′,6-dimethyl-2H-[1,4′-bipyridin]-2-one (D4.11). D4.11 was prepared analogously to D4.1, using B3.3a instead of B3.1a, A9.7 instead of A10.1.

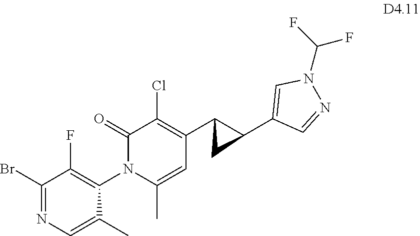

2′,3-dichloro-3′-fluoro-4-((1S,2S)-2-(3-fluoropyridin-2-yl)cyclopropyl)-5′,6-dimethyl-2H-[1,4′-bipyridin]-2-one (D4.12). D4.12 was prepared analogously to D4.1, using B3.2a instead of B3.1a, and A9.14 instead of A10.1.

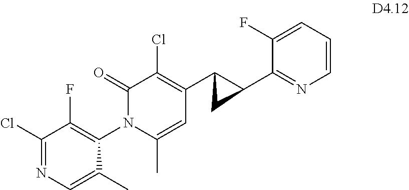

2′,3-dichloro-3′-fluoro-5′,6-dimethyl-4-((1S,2S)-2-(2-(methyl-d3)-2H-1,2,3-triazol-4-yl)cyclopropyl)-2H-[1,4′-bipyridin]-2-one (D4.13). D4.13 was prepared analogously to D4.1, using B3.2a instead of B3.1a, and A9.10 instead of A10.1.

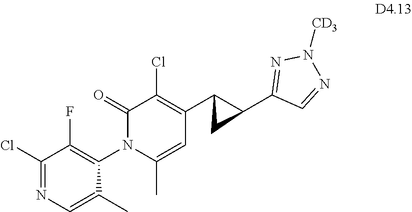

5-chloro-3-(2-chloro-3-fluoro-5-methylpyridin-4-yl)-2-methyl-6-((1S,2S)-2-(2-(methyl-d3)-2H-1,2,3-triazol-4-yl)cyclopropyl)pyrimidin-4(3H)-one (D4.14). D4.14 was prepared analogously to D4.1, using B10.1 instead of B3.1a, and A9.10 instead of A10.1.

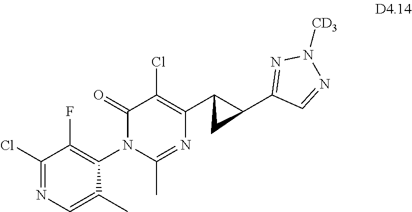

5-chloro-3-(2-chloro-3-fluoro-5-methylpyridin-4-yl)-6-((1S,2S)-2-(5-fluoropyridin-3-yl)cyclopropyl)-2-methylpyrimidin-4(3H)-one (D4.15) D4.15 was prepared analogously to D4.1, using B10.1 instead of B3.1a and A10.32 instead of A10.1.

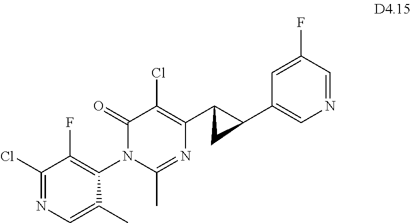

5-chloro-3-(2-chloro-3-fluoro-5-methylpyridin-4-yl)-2-methyl-6-((1S,2S)-2-(1-(methyl-d3)-1H-pyrazol-4-yl)cyclopropyl)pyrimidin-4(3H)-one (D4.16) D4.16 was prepared analogously to D4.1, using B10.1 instead of B3.1a and A9.11 instead of A10.1.

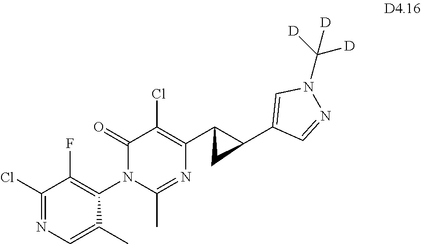

5-chloro-3-(2-chloro-3-fluoro-5-methylpyridin-4-yl)-6-((1S,2S)-2-(5-fluoro-1-(methyl-d3)-1H-pyrazol-4-yl)cyclopropyl)-2-methylpyrimidin-4(3H)-one (D4.17) D4.17 was prepared analogously to D4.1, using B10.1 instead of B3.1a and A9.16 instead of A10.1.

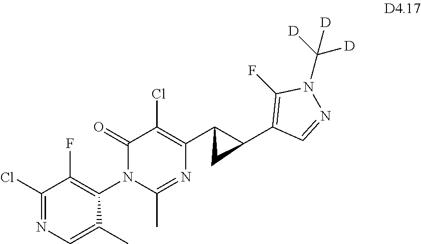

5-chloro-3-(2-chloro-3-fluoro-5-methylpyridin-4-yl)-2-methyl-6-((1S,2S)-2-(4-(methyl-d3)pyrimidin-2-yl)cyclopropyl)pyrimidin-4(3H)-one (D4.18) D4.18 was prepared analogously to D4.1, using B10.1 instead of B3.1a and A9.13 instead of A10.1.

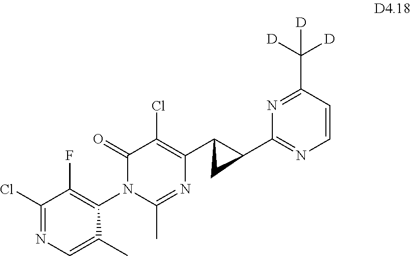

5-chloro-3-((R)-2-chloro-3-fluoro-5-methylpyridin-4-yl)-6-((1S,2S)-2-(5-fluoropyrimidin-2-yl)cyclopropyl)-2-methylpyrimidin-4(3H)-one (D4.19) D4.19 was prepared analogously to D4.1, using B10.1 instead of B3.1a and A9.15 instead of A10.1.

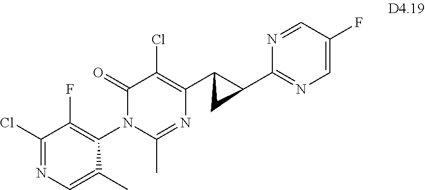

### Preparation of 3-((S)-3-chloro-4-((1S,2S)-2-(4-fluorophenyl)cyclopropyl)-5′,6-dimethyl-2-oxo-2H-[1,4′-bipyridin]-2′-yl)-2-fluorobenzoic acid (D5.1)

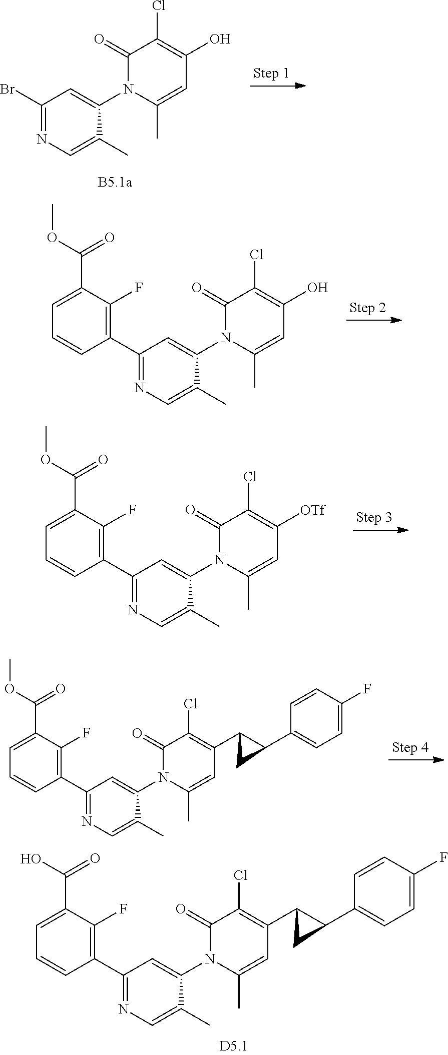

Step 1: 1-(2-bromo-5-methyl-4-pyridyl)-3-chloro-4-hydroxy-6-methyl-pyridin-2-one (2.50 g, 7.59 mmol), methyl 2-fluoro-3-(4,4,5,5-tetramethyl-1,3,2-dioxaborolan-2-yl)benzoate (2.44 g, 8.72 mmol) and cataCXiumPd G4 (0.282 g, 0.379 mmol) were charged to flask and flushed with N₂. 1,2-Dimethoxyethane (30 mL) was added followed by aq. Na₂CO₃ (2M, 15.2 mL, 30.3 mmol). The mixture was sparged with N₂ and then heated 80° C. and stirred for 1 hr. After cooling to room temperature, the reaction was diluted with H₂O and EtOAc and the layers were separated. The aqueous was extracted with EtOAc (3×20 mL), and the organic extracts were combined, dried with MgSO₄, filtered, and concentrated to yield methyl 3-[4-(3-chloro-4-hydroxy-6-methyl-2-oxo-1-pyridyl)-5-methyl-2-pyridyl]-2-fluoro-benzoate, which was used immediately in the next step without further purification.

Step 2: Methyl 3-[4-(3-chloro-4-hydroxy-6-methyl-2-oxo-1-pyridyl)-5-methyl-2-pyridyl]-2-fluoro-benzoate was charged to a flask and dissolved in DCM (25 mL). The mixture was cooled to 0° C. and N,N-diisopropylamine (3.85 g, 29.8 mmol) was added and stirred for 5 mins. Tf₂O (3.15 g, 11.2 mmol) was added dropwise and the reaction was stirred for a further 1 hr. Water was added and the mixture was transferred to a separatory funnel and the layers were separated. The aqueous was extracted with DCM (3×20 mL), and the organic extracts were combined, dried with MgSO₄, filtered, and concentrated. The residue was subjected to silicagel flash chromatography (hexanes-EtOAc) to yield methyl 3-[4-[3-chloro-6-methyl-2-oxo-4-(trifluoromethylsulfonyloxy)-1-pyridyl]-5-methyl-2-pyridyl]-2-fluoro-benzoate. ES/MS: m/z 534.8 [M+H]+.

Step 3: methyl 3-[4-[3-chloro-6-methyl-2-oxo-4-(trifluoromethylsulfonyloxy)-1-pyridyl]-5-methyl-2-pyridyl]-2-fluoro-benzoate (0.1 g, 0.187 mmol), trifluoro((1S,2S)-2-(4-fluorophenyl)cyclopropyl)-λ⁴-borane, potassium salt (70 mg, 0.28 mmol), cesium carbonate (0.18 g, 0.56 mmol), and Pd(dppf)Cl₂-DCM (0.016 g, 0.019 mmol) were charged to a vial and placed under a flow of N₂. PhMe (2 mL) and H₂O (0.5 mL) were added and the reaction was heated to 85° C. for 2.5 hrs. After cooling to room temperature, the reaction was diluted with H₂O and EtOAc and the layers were separated. The aqueous was extracted with EtOAc (3×), and the organic extracts were combined, dried with MgSO₄, filtered, and concentrated to yield methyl 3-[4-[3-chloro-4-[(1S,2S)-2-(4-fluorophenyl)cyclopropyl]-6-methyl-2-oxo-1-pyridyl]-5-methyl-2-pyridyl]-2-fluoro-benzoate, which was used immediately in the next step without further purification.

Step 4: methyl 3-[4-[3-chloro-4-[(1S,2S)-2-(4-fluorophenyl)cyclopropyl]-6-methyl-2-oxo-1-pyridyl]-5-methyl-2-pyridyl]-2-fluoro-benzoate from Step 3 was charged to a vial and dissolved in THF/MeOH (1:1, 2 mL). To the vial, LiOH (1 M, 0.374 mL, 0.374 mmol) was added and the reaction was heated to 50° C. and stirred for 3 hrs. After cooling to room temperature, the reaction was quenched by the addition of sat. aq. NH₄Cl and diluted with EtOAc and the layers were separated. The aqueous was extracted with EtOAc (3×), and the organic extracts were combined, dried with MgSO₄, filtered, and concentrated to yield 3-((S)-3-chloro-4-((1S,2S)-2-(4-fluorophenyl)cyclopropyl)-5′,6-dimethyl-2-oxo-2H-[1,4′-bipyridin]-2′-yl)-2-fluorobenzoic acid (D5.1).

### Preparation 3-(3-chloro-3′-fluoro-4-((1S,2S)-2-(4-fluorophenyl)cyclopropyl)-5′,6-dimethyl-2-oxo-2H-[1,4′-bipyridin]-2′-yl)-2-fluorobenzoic acid (D5.2)

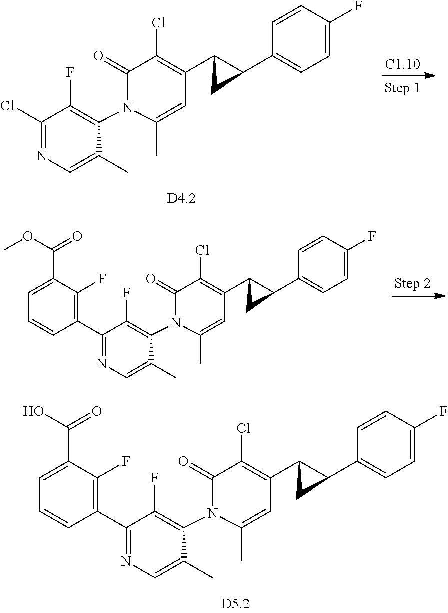

Step 1. Step 1 in the preparation of D5.2 was analogous to Step 1 in D5.1 synthesis, using D4.2 instead of B5.1a.

Step 2. Step 2 in the preparation of D5.2 was analogous to Step 4 in D5.1 synthesis. 3-(3-chloro-4-((1S,2S)-2-(1-(difluoromethyl)-1H-pyrazol-4-yl)cyclopropyl)-5′,6-dimethyl-2-oxo-2H-[1,4′-bipyridin]-2′-yl)-2-fluorobenzoic acid (D5.3) Prepared analogously to D5.2, using D4.3 instead of D4.2.

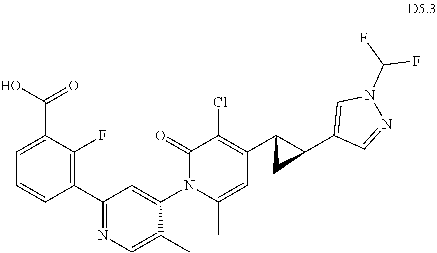

3-(3-chloro-4-((1S,2S)-2-(1-(difluoromethyl)-1H-pyrazol-4-yl)cyclopropyl)-3′-fluoro-5′,6-dimethyl-2-oxo-2H-[1,4′-bipyridin]-2′-yl)-2-fluorobenzoic acid (D5.4). D5.4 was prepared analogously to D5.2, using D4.4 instead of D4.2.

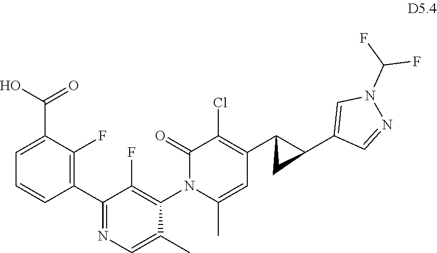

3-(3-chloro-4-((1S,2S)-2-(5-chloropyridin-3-yl)cyclopropyl)-5′,6-dimethyl-2-oxo-2H-[1,4′-bipyridin]-2′-yl)-2-fluorobenzoic acid (D5.5). D5.5 was prepared analogously to D5.2, using D4.5 instead of D4.2

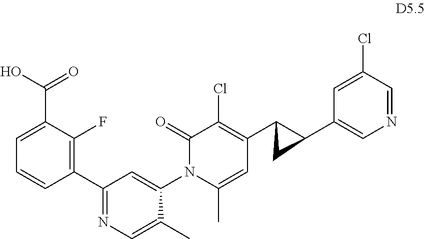

3-(3-chloro-4-((1S,2S)-2-(5-chloropyridin-3-yl)cyclopropyl)-3′-fluoro-5′,6-dimethyl-2-oxo-2H-[1,4′-bipyridin]-2′-yl)-2-fluorobenzoic acid (D5.6). D5.6 was prepared analogously to D5.2, using D4.6 instead of D4.2.

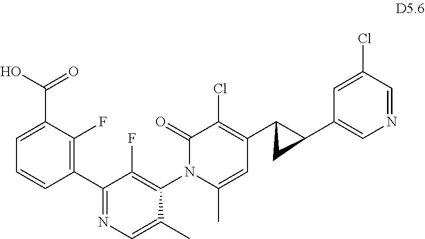

3-(3-chloro-4-((1S,2S)-2-(5-fluoropyridin-3-yl)cyclopropyl)-5′,6-dimethyl-2-oxo-2H-[1,4′-bipyridin]-2′-yl)-2-fluorobenzoic acid (D5.7). D5.7 was prepared analogously to D5.2, using D4.7 instead of D4.2.

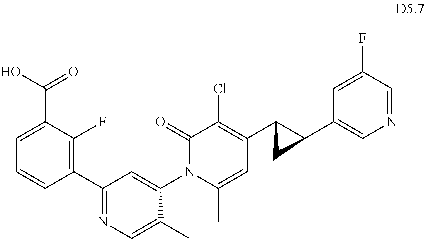

3-(3-chloro-3′-fluoro-4-((1S,2S)-2-(5-fluoropyridin-3-yl)cyclopropyl)-5′,6-dimethyl-2-oxo-2H-[1,4′-bipyridin]-2′-yl)-2-fluorobenzoic acid (D5.8). D5.8 was prepared analogously to D5.2, using D4.8 instead of D4.2.

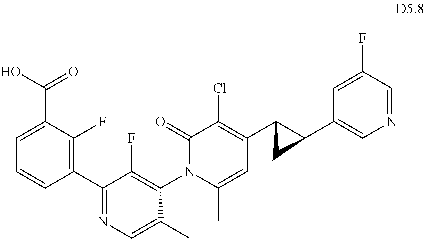

4-(3-chloro-4-((1S,2S)-2-(5-chloropyridin-3-yl)cyclopropyl)-5′,6-dimethyl-2-oxo-2H-[1,4′-bipyridin]-2′-yl)-3-fluorothiophene-2-carboxylic acid (D5.9). D5.9 was prepared analogously to D5.2, using D4.5 instead of D4.2 and C7.8 instead of C1.10.

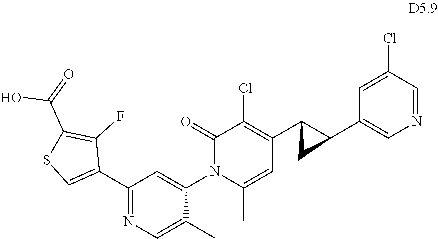

5-(3-chloro-4-((1S,2S)-2-(5-chloropyridin-3-yl)cyclopropyl)-5′,6-dimethyl-2-oxo-2H-[1,4′-bipyridin]-2′-yl)-4-fluorothiophene-3-carboxylic acid (D5.10). D5.10 was prepared analogously to D5.2, using D4.5 instead of D4.2 and C7.9 instead of C1.10.

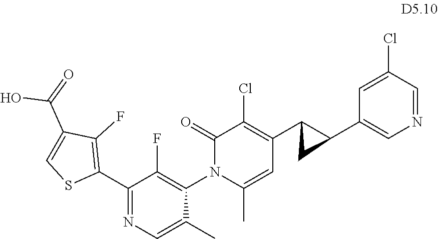

4-(3-chloro-4-((1S,2S)-2-(4-fluorophenyl)cyclopropyl)-5′,6-dimethyl-2-oxo-2H-[1,4′-bipyridin]-2′-yl)-1-methyl-1H-imidazole-2-carboxylic acid (D5.11) D5.11 was prepared analogously to D5.2, using D4.1 instead of D4.2 and C7.3 instead of C1.10.

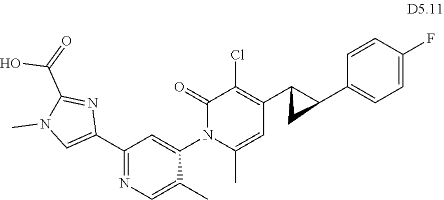

5-(3-chloro-4-((1S,2S)-2-(4-fluorophenyl)cyclopropyl)-5′,6-dimethyl-2-oxo-2H-[1,4′-bipyridin]-2′-yl)-1-methyl-1H-imidazole-2-carboxylic acid (D5.12). D5.12 was prepared analogously to D5.2, using D4.1 instead of D4.2 and C7.4 instead of C1.10.

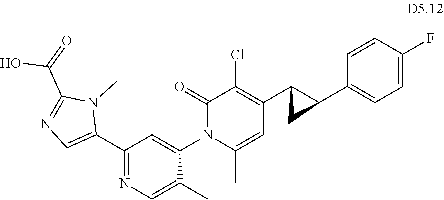

6-(3-chloro-4-((1S,2S)-2-(4-fluorophenyl)cyclopropyl)-5′,6-dimethyl-2-oxo-2H-[1,4′-bipyridin]-2′-yl)-5-fluoro-1-(tetrahydro-2H-pyran-2-yl)-1H-indazole-4-carboxylic acid (D5.13). D5.13 was prepared analogously to D5.2, using D4.1 instead of D4.2 and C7.5 instead of C1.10.

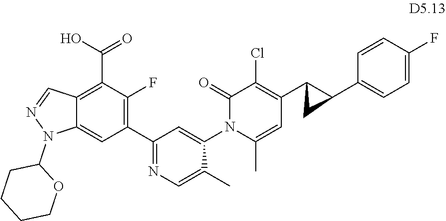

3-(3-chloro-4-((1S,2S)-2-(5-(difluoromethyl)pyridin-3-yl)cyclopropyl)-3′-fluoro-5′,6-dimethyl-2-oxo-2H-[1,4′-bipyridin]-2′-yl)-2-fluorobenzoic acid (D5.14). D5.14 was prepared analogously to D5.2, using D4.9 instead of D4.2.

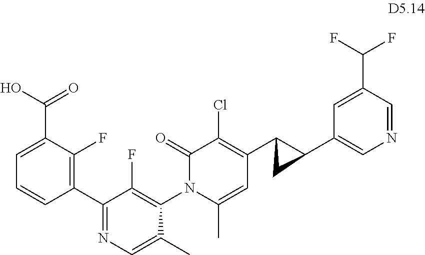

### Preparation of 1-(3-chloro-4-((1S,2S)-2-(4-fluorophenyl)cyclopropyl)-5′,6-dimethyl-2-oxo-2H-[1,4′-bipyridin]-2′-yl)-N,N-dimethyl-1H-pyrazole-3-carboxamide (D5.19)

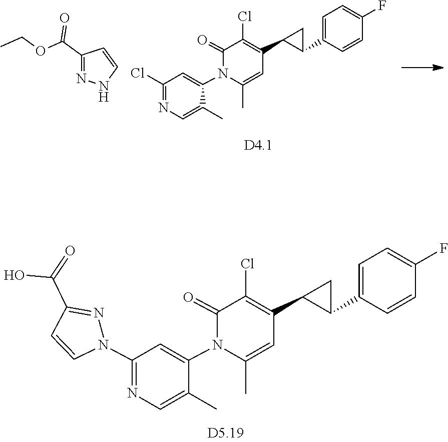

Methyl 1H-pyrazole-3-carboxylate (88 mg, 0.69 mmol), 2′,3-dichloro-4-((1S,2S)-2-(4-fluorophenyl)cyclopropyl)-5′,6-dimethyl-2H-[1,4′-bipyridin]-2-one (D4.1) (56 mg, 0.14 mmol), and cesium carbonate (136 mg, 0.42 mmol) were charged to a vial and suspended in DMF (3.0 mL). The mixture was stirred at 130° C. for 14 hours then cooled and concentrated under reduced pressure. The residue was subjected to reverse phase HPLC (Silicycle SiliaChrom SB C18, 5 μm, 150 Å, 30×250 mm; 0.1% TFA in MeCN-0.1% TFA in water) to give 1-(3-chloro-4-((1S,2S)-2-(4-fluorophenyl)cyclopropyl)-5′,6-dimethyl-2-oxo-2H-[1,4′-bipyridin]-2′-yl)-1H-pyrazole-3-carboxylic acid (D5.19).

2′-(3-amino-2-fluorophenyl)-3-chloro-4-((1S,2S)-2-(5-fluoropyridin-3-yl)cyclopropyl)-5′,6-dimethyl-2H-[1,4′-bipyridin]-2-one (D5.15) D5.15 was prepared analogously to D5.2, using D4.7 instead of D4.2 and C7.14 instead of C1.10, followed by treatment with 1:1 TFA/DCM for 15 min at room temperature.

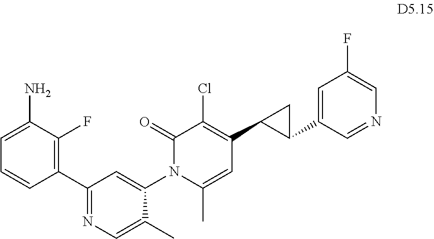

2′-(3-amino-2-fluorophenyl)-3-chloro-4-((1S,2S)-2-(5-chloropyridin-3-yl)cyclopropyl)-5′,6-dimethyl-2H-[1,4′-bipyridin]-2-one (D5.16) D5.16 was prepared analogously to D5.2, using D4.5 instead of D4.2 and C7.14 instead of C1.10, followed by treatment with 1:1 TFA/DCM for 15 min at room temperature.

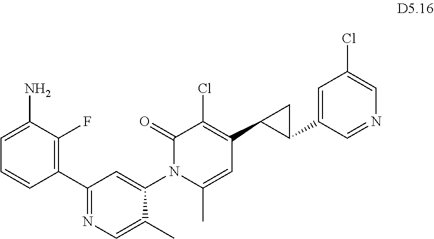

3-chloro-2″,3″-difluoro-4-((1S,2S)-2-(4-fluorophenyl)cyclopropyl)-5′,6-dimethyl-2H-[1,4′:2′,4″-terpyridin]-2-one (D5.17) D5.17 was prepared analogously to D5.2, using (rac)-D4.1 instead of D4.2 and C1.22 instead of C1.10.

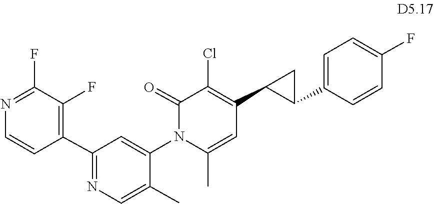 [SMILES: Cc1cnc(-c2ccnc(F)c2F)cc1-n1c(C)cc([C@H]2C[C@@H]2c2ccc(F)cc2)c(Cl)c1=O]

3-chloro-2″,3″-difluoro-4-((1S,2S)-2-(5-fluoropyridin-3-yl)cyclopropyl)-5′,6-dimethyl-2H-[1,4′:2′,4″-terpyridin]-2-one (D5.18) D5.18 was prepared analogously to D5.2, using D4.7 instead of D4.2 and C1.22 instead of C1.10.

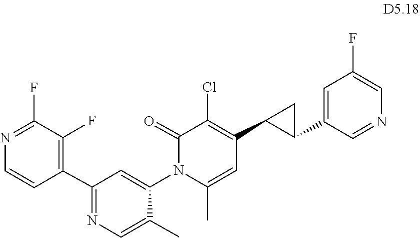

3-chloro-2″,3′,3″-trifluoro-4-((1S,2S)-2-(5-fluoropyridin-3-yl)cyclopropyl)-5′,6-dimethyl-2H-[1,4′:2′,4″-terpyridin]-2-one (D5.19)D5.19 was prepared analogously to D5.2, using D4.8 instead of D4.2 and C1.22 instead of C1.10.

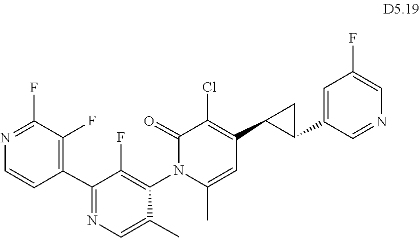

### Preparation of 3-chloro-2″-(3,5-dimethyl-1H-1,2,4-triazol-1-yl)-3′,3″-difluoro-5′,6-dimethyl-4-((1S,2S)-2-(4,4,5,5-tetramethyl-1,3,2-dioxaborolan-2-yl)cyclopropyl)-2H-[1,4′:2′,4″-terpyridin]-2-one (D6.1)

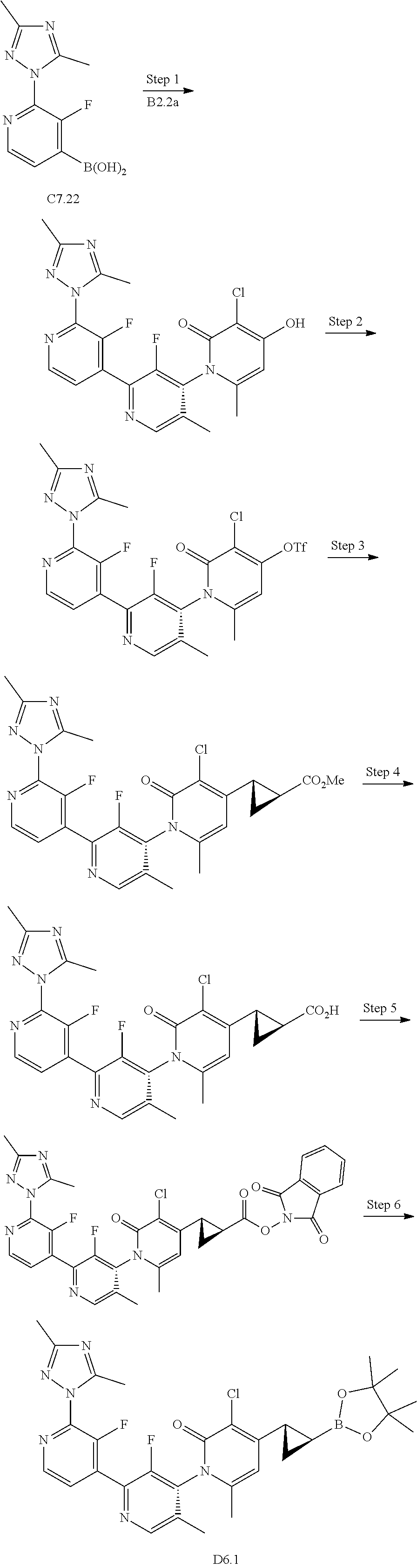

Step 1. 3-Chloro-2″-(3,5-dimethyl-1H-1,2,4-triazol-1-yl)-3′,3″-difluoro-4-hydroxy-5′,6-dimethyl-2H-[1,4′:2′,4″-terpyridin]-2-one was prepared following the procedure outlined in step 1 of the synthesis of D1.1a, starting from B2.2a instead of B5.1a and C7.22 instead of C1.8, using PdCl₂(dppf) instead of SPhos Pd G4 and Cs₂CO₃ instead of Na₂CO₃.

Step 2. 3-Chloro-2″-(3,5-dimethyl-1H-1,2,4-triazol-1-yl)-3′,3″-difluoro-5′,6-dimethyl-2-oxo-2H-[1,4′:2′,4″-terpyridin]-4-yl trifluoromethanesulfonate was prepared following the procedure outlined in step 2 of the synthesis of D1.1a using PhN(Tf)₂ instead of Tf₂O in Step 2.

Step 3. Methyl (1S,2S)-2-(3-chloro-2″-(3,5-dimethyl-1H-1,2,4-triazol-1-yl)-3′,3″-difluoro-5′,6-dimethyl-2-oxo-2H-[1,4′:2′,4″-terpyridin]-4-yl)cyclopropane-1-carboxylate was prepared analogously to B6.1.

Step 4. (1S,2S)-2-(3-chloro-2″-(3,5-dimethyl-1H-1,2,4-triazol-1-yl)-3′,3″-difluoro-5′,6-dimethyl-2-oxo-2H-[1,4′:2′,4″-terpyridin]-4-yl)cyclopropane-1-carboxylic acid was prepared following the procedure outlined in step 2 of the synthesis of D3.1.

Step 5. 1,3-dioxoisoindolin-2-yl (1S,2S)-2-(3-chloro-2″-(3,5-dimethyl-1H-1,2,4-triazol-1-yl)-3′,3″-difluoro-5′,6-dimethyl-2-oxo-2H-[1,4′:2′,4″-terpyridin]-4-yl)cyclopropane-1-carboxylate was prepared following the procedure outlined in step 3 of the synthesis of D3.1.

Step 6. D6.1 was prepared following the procedure outlined in step 4 of the synthesis of D3.1 using EtOAc instead of PhCF₃.

3-chloro-2′-(3-(3,5-dimethyl-1H-1,2,4-triazol-1-yl)-2-fluorophenyl)-3′-fluoro-5′,6-dimethyl-4-((1S,2S)-2-(4,4,5,5-tetramethyl-1,3,2-dioxaborolan-2-yl)cyclopropyl)-2H-[1,4′-bipyridin]-2-one (D6.2). D6.2 was prepared analogously to D6.1 starting from C13.3 instead of C7.22.

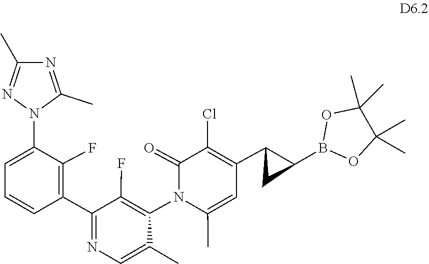

3-chloro-3′-fluoro-2′-(2-fluoro-3-(1-methyl-1H-1,2,4-triazol-5-yl)phenyl)-5′,6-dimethyl-4-((1S,2S)-2-(4,4,5,5-tetramethyl-1,3,2-dioxaborolan-2-yl)cyclopropyl)-2H-[1,4′-bipyridin]-2-one (D6.3). D6.3 was prepared analogously to D6.1 starting from C9.3 instead of C7.22.

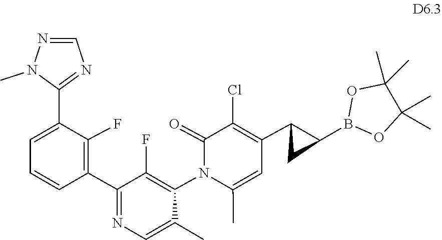

### Final Procedures

### Example 1.1: (rac)-4-((1S,2R)-2-benzylcyclopropyl)-3-chloro-2′-(2-(2-hydroxypropan-2-yl)pyrimidin-4-yl)-5′,6-dimethyl-2H-[1,4′-bipyridin]-2-one

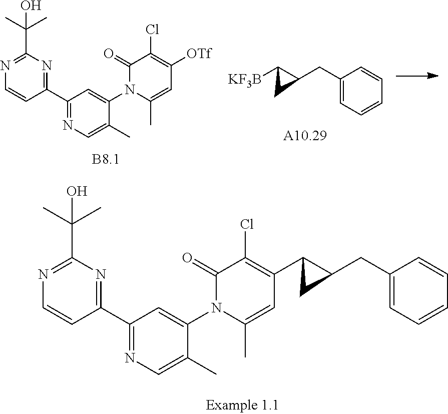

A vial was charged with 3-chloro-2′-(2-(2-hydroxypropan-2-yl)pyrimidin-4-yl)-5′,6-dimethyl-2-oxo-2H-[1,4′-bipyridin]-4-yl trifluoromethanesulfonate (B8.1) (30 mg, 0.058 mmol), Pd(PPh₃)₄ (6.6 mg, 0.058 mmol), Cs₂CO₃ (57 mg, 0.17 mmol), and (rac)-((1S,2R)-2-benzylcyclopropyl)trifluoro-λ⁴-borane, potassium salt (10.29) (21 mg, 0.087 mmol), PhMe (0.5 mL) and water (0.1 mL). The mixture was sparged with argon for 2 minutes, before the vial was capped and the mixture heated to 90° C. for 2 hrs. The mixture was cooled to ambient temperature, filtered over celite, concentrated and subject flash column chromatography (SiO₂, hexanes-EtOAc-MeOH), followed by reverse phase HPLC (Silicycle SiliaChrom SB C18, 5 μm, 150 Å, 30×250 mm; 0.1% TFA in MeCN-0.1% TFA in water) to afford (rac)-4-((1S,2R)-2-benzylcyclopropyl)-3-chloro-2′-(2-(2-hydroxypropan-2-yl)pyrimidin-4-yl)-5′,6-dimethyl-2H-[1,4′-bipyridin]-2-one (Example 1.1).

The following compounds in Table 1 were prepared analogously to Example 1.1 modified as

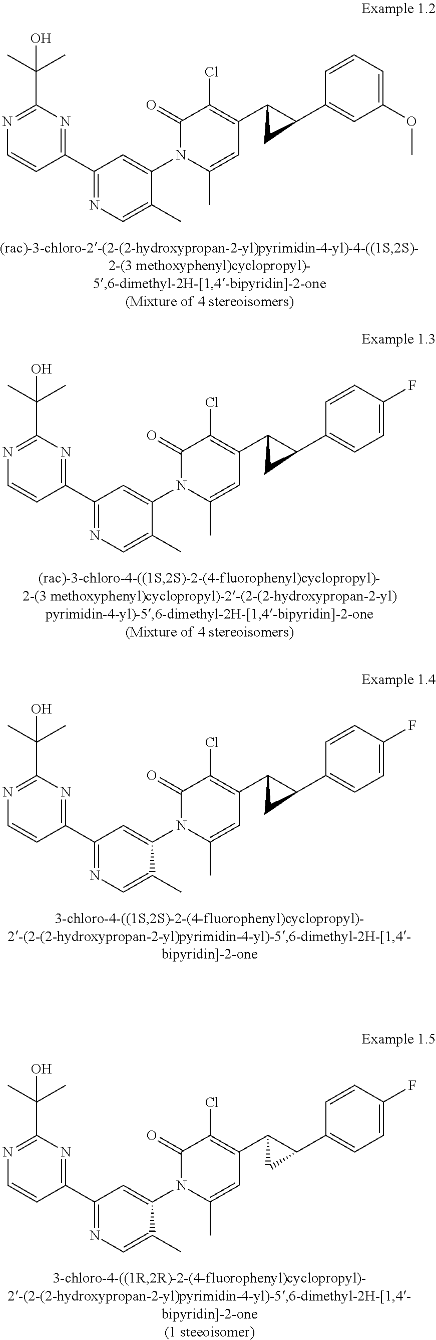

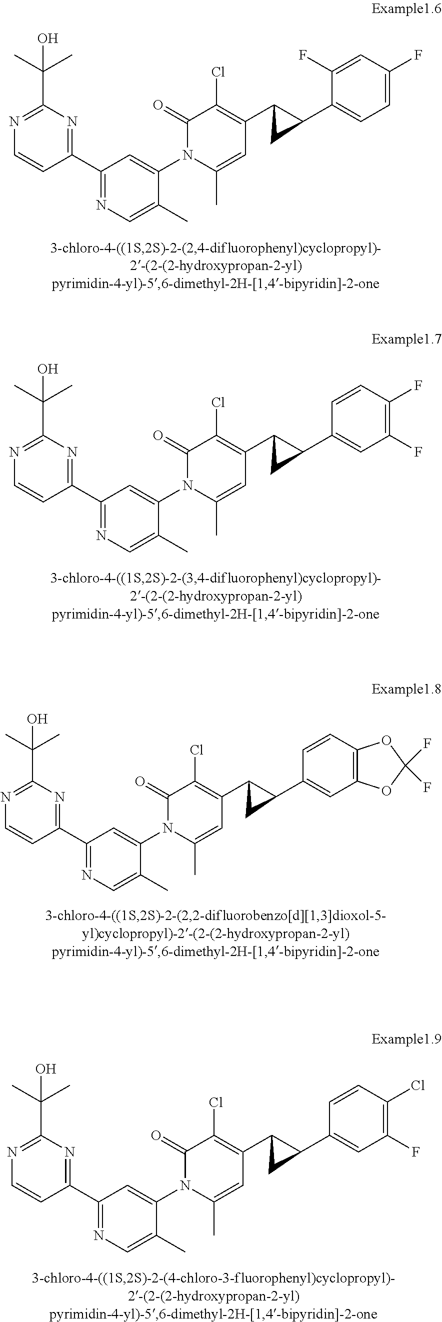 [SMILES: Cc1cnc(-c2ccnc(C(C)(C)O)n2)cc1-n1c(C)cc([C@H]2C[C@@H]2c2ccc(F)cc2F)c(Cl)c1=O ; Cc1cnc(-c2ccnc(C(C)(C)O)n2)cc1-n1c(C)cc([C@H]2C[C@@H]2c2ccc(F)c(F)c2)c(Cl)c1=O ; Cc1cnc(-c2ccnc(C(C)(C)O)n2)cc1-n1c(C)cc([C@H]2C[C@@H]2c2ccc3c(c2)OC(F)(F)O3)c(Cl)c1=O ; Cc1cnc(-c2ccnc(C(C)(C)O)n2)cc1-n1c(C)cc([C@H]2C[C@@H]2c2ccc(Cl)c(F)c2)c(Cl)c1=O]

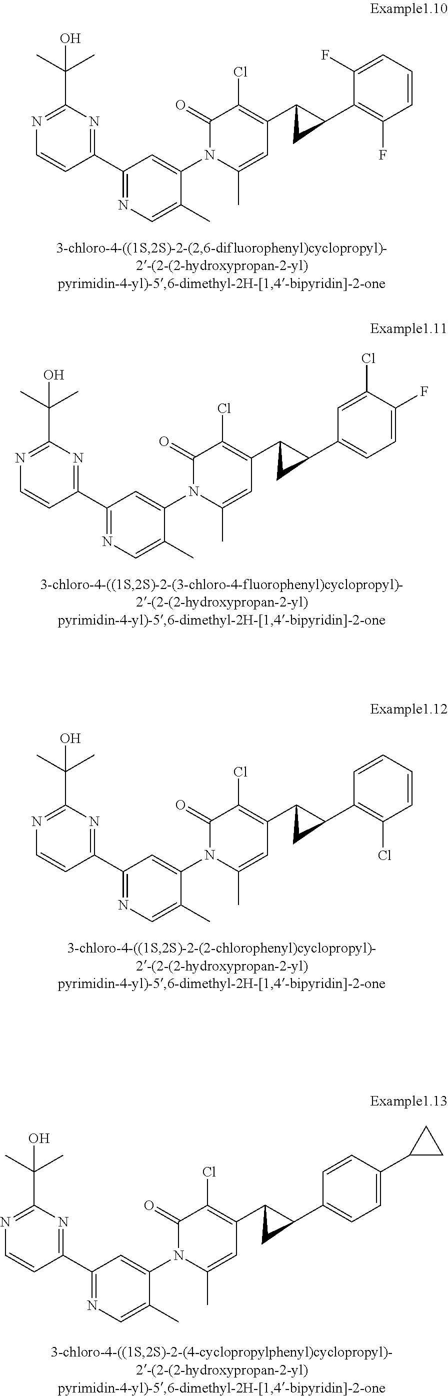 [SMILES: Cc1cnc(-c2ccnc(C(C)(C)O)n2)cc1-n1c(C)cc([C@H]2C[C@@H]2c2c(F)cccc2F)c(Cl)c1=O ; Cc1cnc(-c2ccnc(C(C)(C)O)n2)cc1-n1c(C)cc([C@H]2C[C@@H]2c2ccc(F)c(Cl)c2)c(Cl)c1=O ; Cc1cnc(-c2ccnc(C(C)(C)O)n2)cc1-n1c(C)cc([C@H]2C[C@@H]2c2ccccc2Cl)c(Cl)c1=O ; Cc1cnc(-c2ccnc(C(C)(C)O)n2)cc1-n1c(C)cc([C@H]2C[C@@H]2c2ccc(C3CC3)cc2)c(Cl)c1=O]

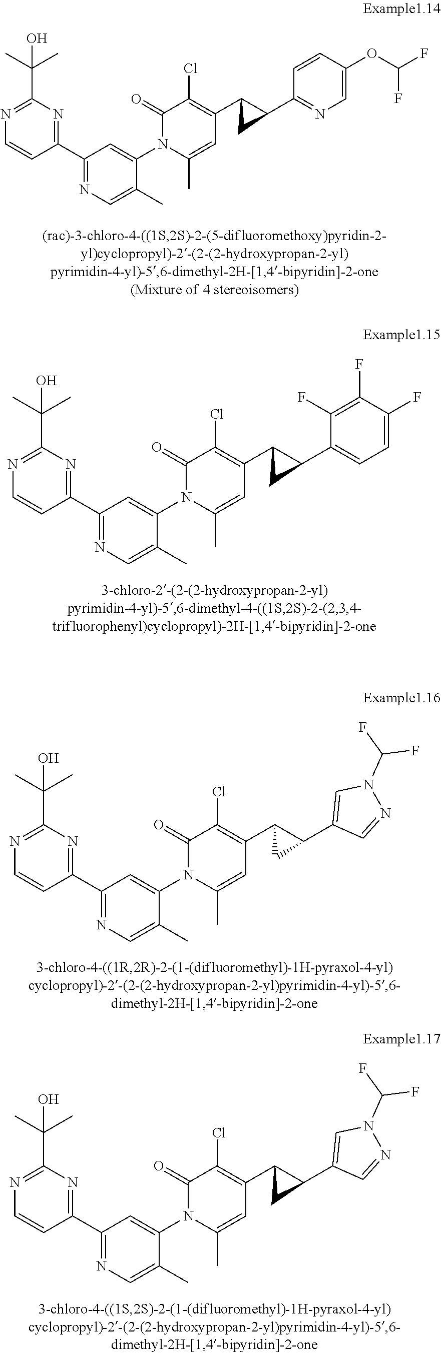 [SMILES: Cc1cnc(-c2ccnc(C(C)(C)O)n2)cc1-n1c(C)cc([C@H]2C[C@@H]2c2ccc(OC(F)F)cn2)c(Cl)c1=O ; Cc1cnc(-c2ccnc(C(C)(C)O)n2)cc1-n1c(C)cc([C@H]2C[C@@H]2c2ccc(F)c(F)c2F)c(Cl)c1=O ; Cc1cnc(-c2ccnc(C(C)(C)O)n2)cc1-n1c(C)cc([C@@H]2C[C@H]2c2cnn(C(F)F)c2)c(Cl)c1=O ; Cc1cnc(-c2ccnc(C(C)(C)O)n2)cc1-n1c(C)cc([C@H]2C[C@@H]2c2cnn(C(F)F)c2)c(Cl)c1=O]

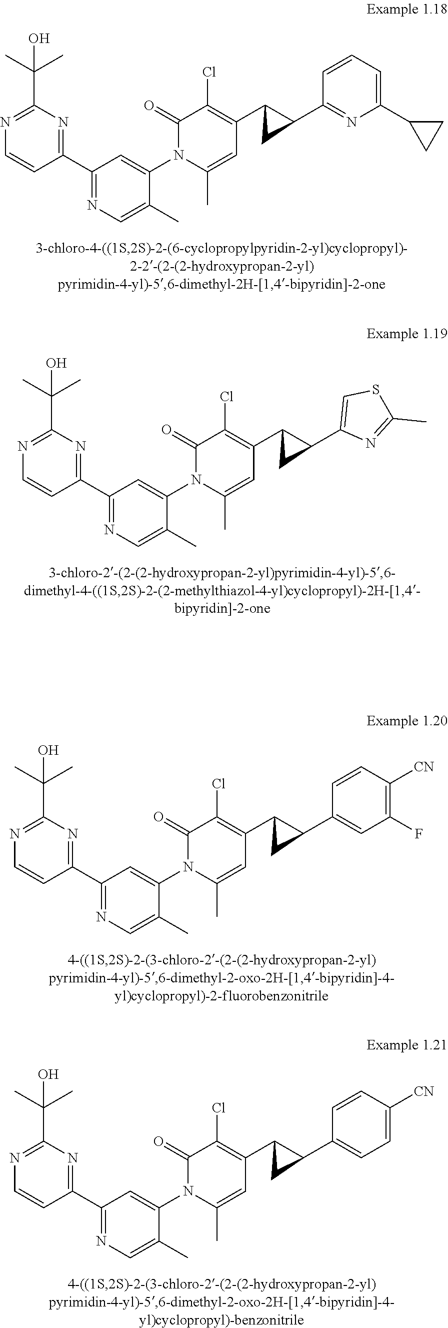

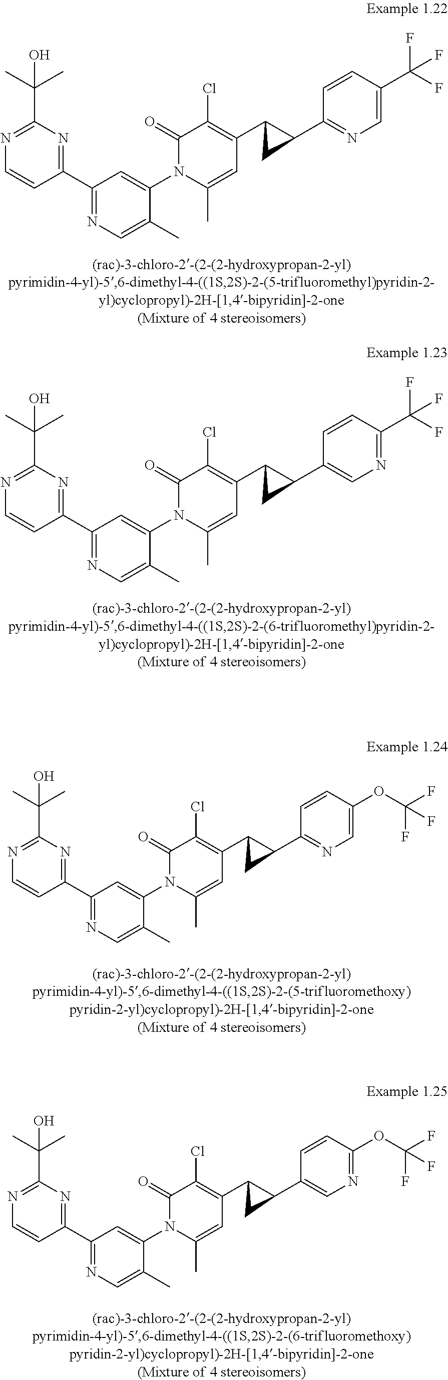 [SMILES: Cc1cnc(-c2ccnc(C(C)(C)O)n2)cc1-n1c(C)cc([C@H]2C[C@@H]2c2ccc(C(F)(F)F)cn2)c(Cl)c1=O ; Cc1cnc(-c2ccnc(C(C)(C)O)n2)cc1-n1c(C)cc([C@H]2C[C@@H]2c2ccc(C(F)(F)F)nc2)c(Cl)c1=O ; Cc1cnc(-c2ccnc(C(C)(C)O)n2)cc1-n1c(C)cc([C@H]2C[C@@H]2c2ccc(OC(F)(F)F)cn2)c(Cl)c1=O ; Cc1cnc(-c2ccnc(C(C)(C)O)n2)cc1-n1c(C)cc([C@H]2C[C@@H]2c2ccc(OC(F)(F)F)nc2)c(Cl)c1=O]

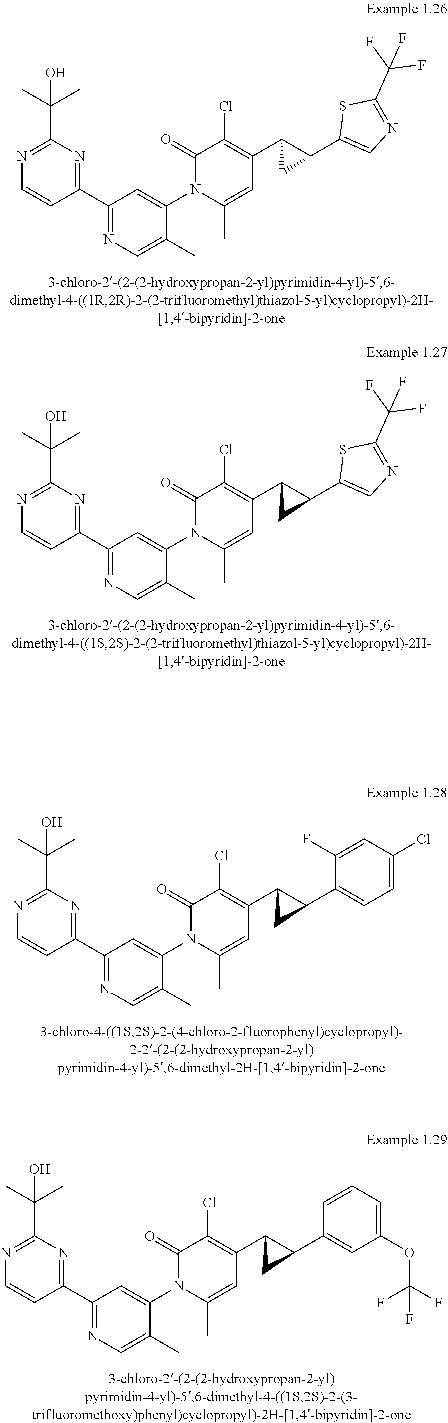 [SMILES: Cc1cnc(-c2ccnc(C(C)(C)O)n2)cc1-n1c(C)cc([C@@H]2C[C@H]2c2cnc(C(F)(F)F)s2)c(Cl)c1=O ; Cc1cnc(-c2ccnc(C(C)(C)O)n2)cc1-n1c(C)cc([C@H]2C[C@@H]2c2cnc(C(F)(F)F)s2)c(Cl)c1=O ; Cc1cnc(-c2ccnc(C(C)(C)O)n2)cc1-n1c(C)cc([C@H]2C[C@@H]2c2ccc(Cl)cc2F)c(Cl)c1=O ; Cc1cnc(-c2ccnc(C(C)(C)O)n2)cc1-n1c(C)cc([C@H]2C[C@@H]2c2cccc(OC(F)(F)F)c2)c(Cl)c1=O]

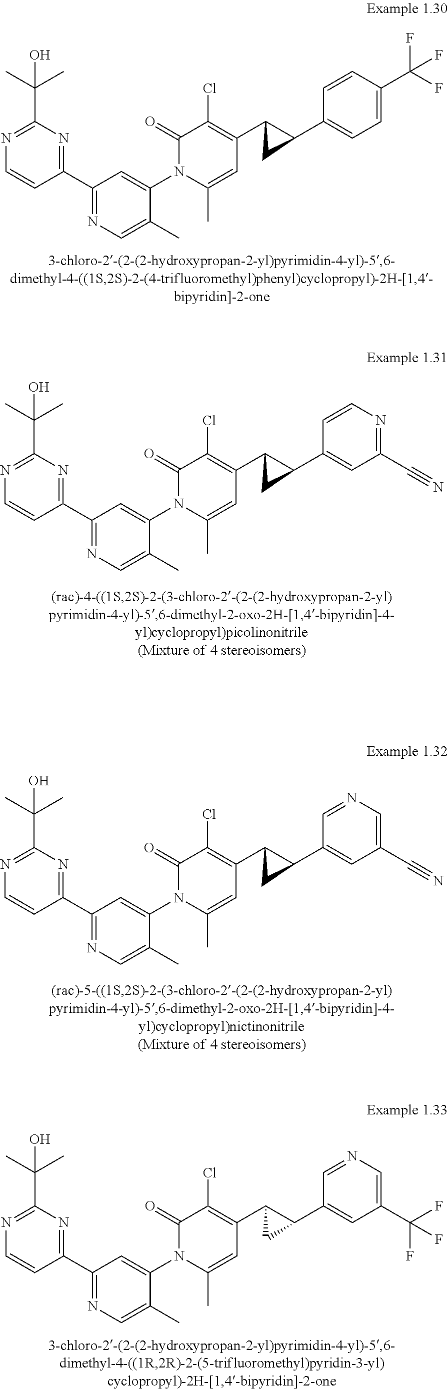 [SMILES: Cc1cnc(-c2ccnc(C(C)(C)O)n2)cc1-n1c(C)cc([C@H]2C[C@@H]2c2ccc(C(F)(F)F)cc2)c(Cl)c1=O ; Cc1cnc(-c2ccnc(C(C)(C)O)n2)cc1-n1c(C)cc([C@H]2C[C@@H]2c2ccnc(C#N)c2)c(Cl)c1=O ; Cc1cnc(-c2ccnc(C(C)(C)O)n2)cc1-n1c(C)cc([C@H]2C[C@@H]2c2cncc(C#N)c2)c(Cl)c1=O ; Cc1cnc(-c2ccnc(C(C)(C)O)n2)cc1-n1c(C)cc([C@@H]2C[C@H]2c2cncc(C(F)(F)F)c2)c(Cl)c1=O]

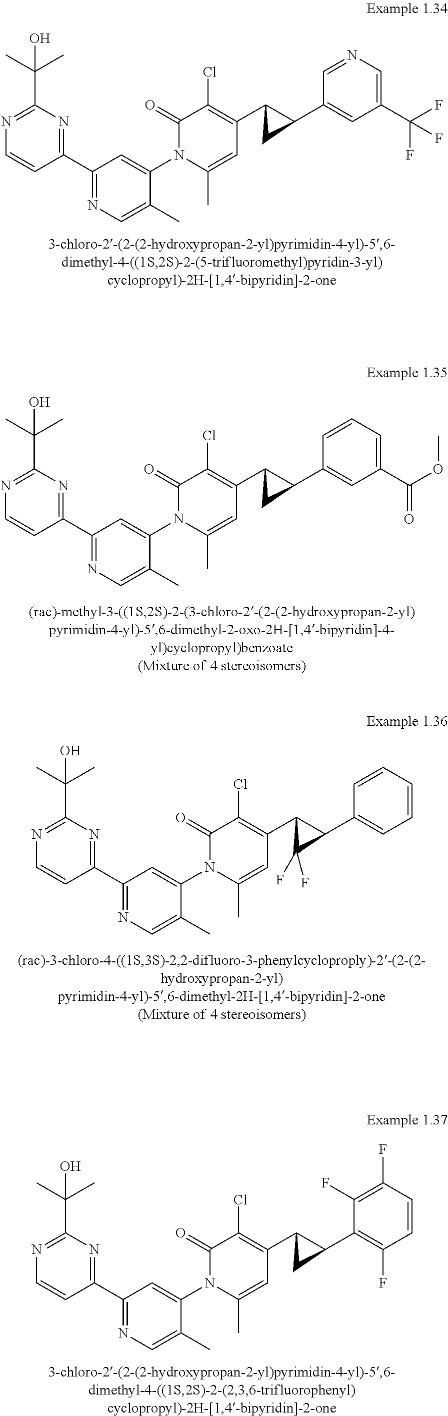

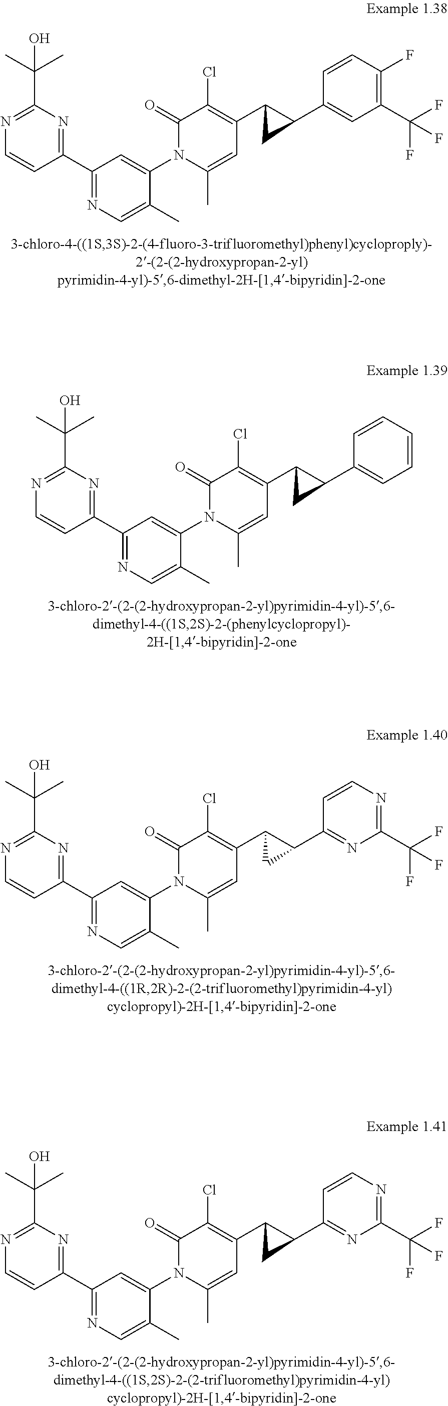 [SMILES: Cc1cnc(-c2ccnc(C(C)(C)O)n2)cc1-n1c(C)cc([C@H]2C[C@@H]2c2ccc(F)c(C(F)(F)F)c2)c(Cl)c1=O ; Cc1cnc(-c2ccnc(C(C)(C)O)n2)cc1-n1c(C)cc([C@H]2C[C@@H]2c2ccccc2)c(Cl)c1=O ; Cc1cnc(-c2ccnc(C(C)(C)O)n2)cc1-n1c(C)cc([C@@H]2C[C@H]2c2ccnc(C(F)(F)F)n2)c(Cl)c1=O ; Cc1cnc(-c2ccnc(C(C)(C)O)n2)cc1-n1c(C)cc([C@H]2C[C@@H]2c2ccnc(C(F)(F)F)n2)c(Cl)c1=O]

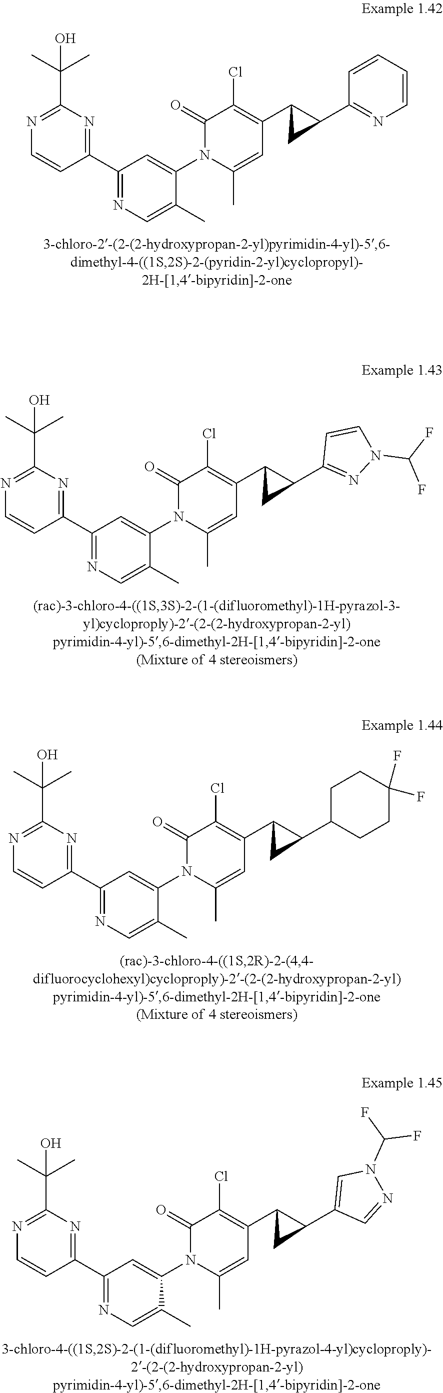

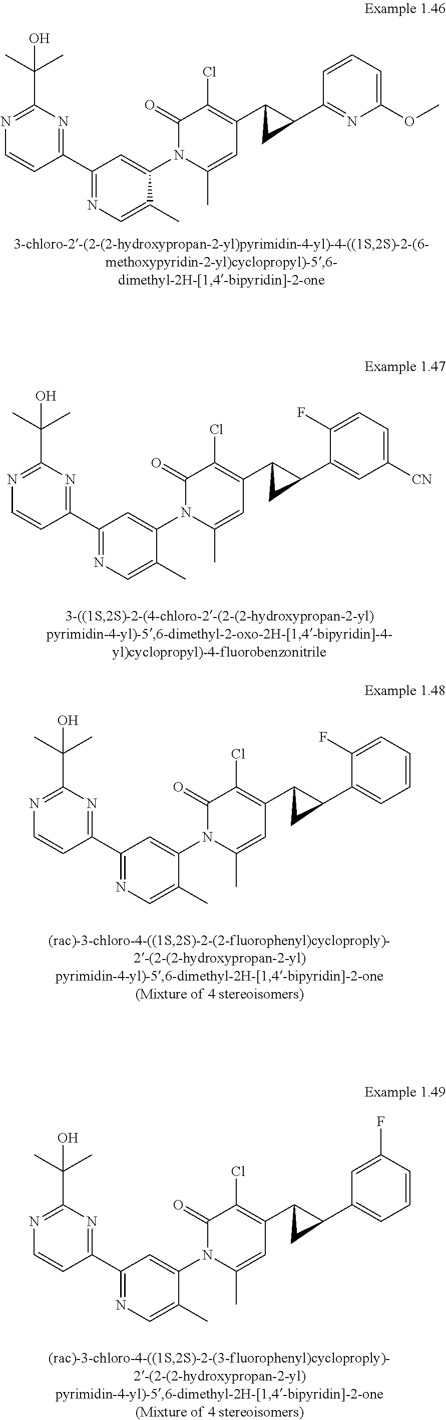

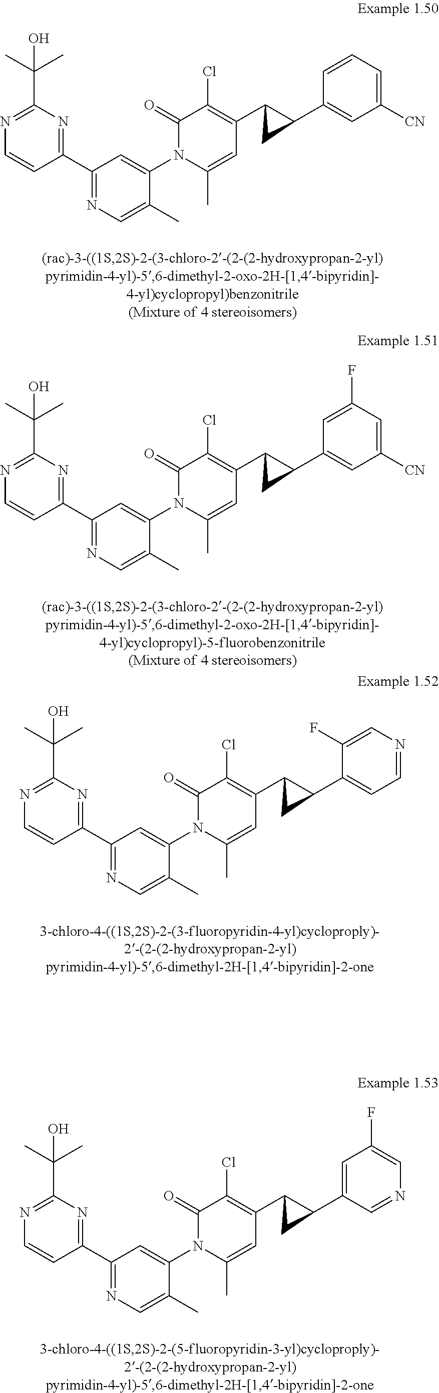 [SMILES: Cc1cnc(-c2ccnc(C(C)(C)O)n2)cc1-n1c(C)cc([C@H]2C[C@@H]2c2cccc(C#N)c2)c(Cl)c1=O ; Cc1cnc(-c2ccnc(C(C)(C)O)n2)cc1-n1c(C)cc([C@H]2C[C@@H]2c2cc(F)cc(C#N)c2)c(Cl)c1=O ; Cc1cnc(-c2ccnc(C(C)(C)O)n2)cc1-n1c(C)cc([C@H]2C[C@@H]2c2ccncc2F)c(Cl)c1=O ; Cc1cnc(-c2ccnc(C(C)(C)O)n2)cc1-n1c(C)cc([C@H]2C[C@@H]2c2cncc(F)c2)c(Cl)c1=O]

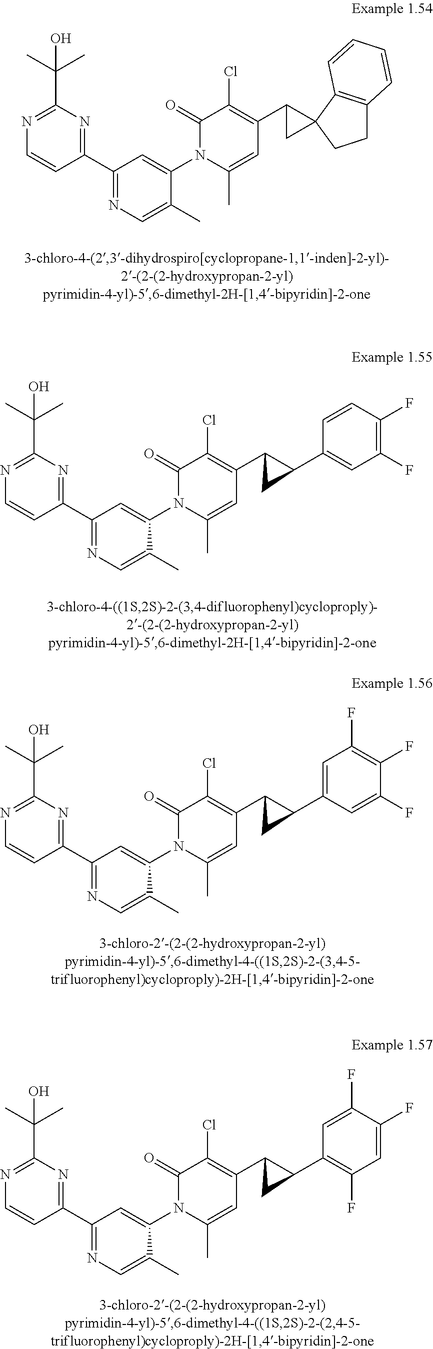

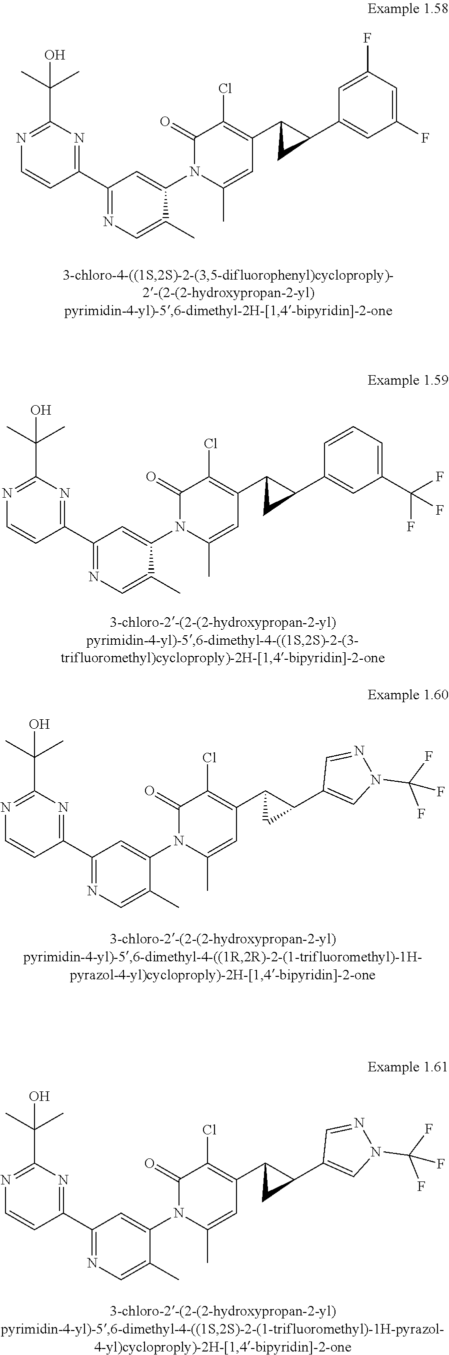

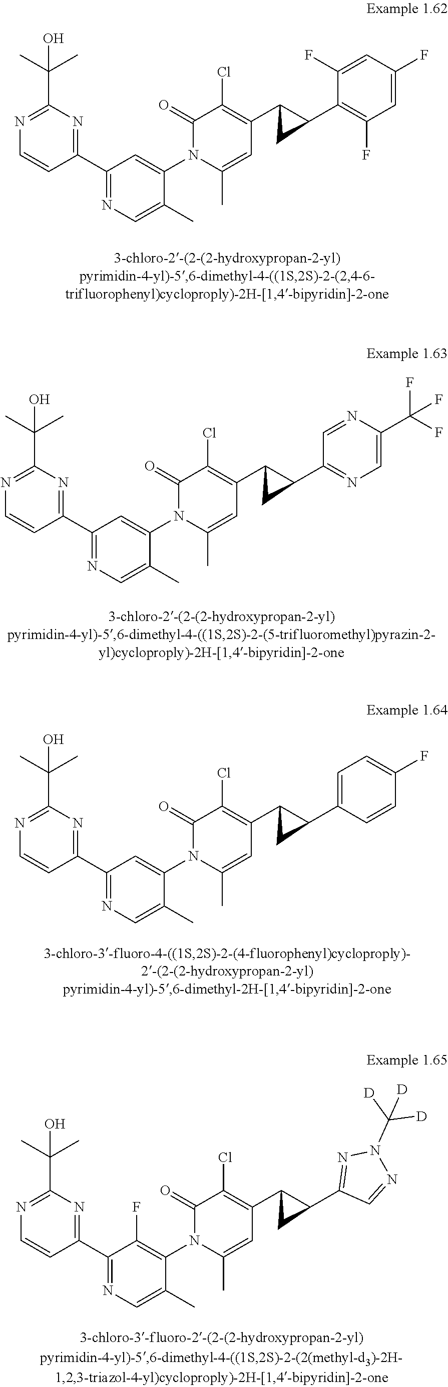 [SMILES: Cc1cnc(-c2ccnc(C(C)(C)O)n2)cc1-n1c(C)cc([C@H]2C[C@@H]2c2c(F)cc(F)cc2F)c(Cl)c1=O ; Cc1cnc(-c2ccnc(C(C)(C)O)n2)cc1-n1c(C)cc([C@H]2C[C@@H]2c2cnc(C(F)(F)F)cn2)c(Cl)c1=O ; Cc1cnc(-c2ccnc(C(C)(C)O)n2)cc1-n1c(C)cc([C@H]2C[C@@H]2c2ccc(F)cc2)c(Cl)c1=O ; [2H]C([2H])([2H])n1ncc([C@H]2C[C@@H]2c2cc(C)n(-c3c(C)cnc(-c4ccnc(C(C)(C)O)n4)c3F)c(=O)c2Cl)n1]

TABLE 1
Procedures analogous to Example 1.1 Synthesis

| Example | | Variations from Example 1.1 |
|---|---|---|
| Number | Instead of A10.29 | Synthesis |
| 1.2 | A10.4 | |
| 1.3 | A10.2 | |
| 1.4 | A10.2 | B8.1a was used instead of B8.1 |
| 1.5 | A10.3 | B8.1a was used instead of B8.1 |
| 1.6 | A10.25 | |
| 1.7 | A10.24 | |
| 1.8 | A10.5 | |
| 1.9 | A10.22 | PdCl₂(dppf) instead of Pd(PPh₃)₄ |
| 1.10 | A10.23 | PdCl₂(dppf) instead of Pd(PPh₃)₄ |
| 1.11 | A7.1 | |
| 1.12 | A11.9 | PdCl₂(dppf) instead of Pd(PPh₃)₄ |
| 1.13 | A12.1 | PdCl₂(dppf) instead of Pd(PPh₃)₄ |
| 1.14 | A1.4 | PdCl₂(dppf) instead of Pd(PPh₃)₄ |
| 1.15 | A1.5 | |
| 1.16 | A1.6 | PdCl₂(dppf) instead of Pd(PPh₃)₄ |
| 1.17 | A9.7 | PdCl₂(dppf) instead of Pd(PPh₃)₄ |
| 1.18 | A10.27 | |
| 1.19 | A10.28 | |
| 1.20 | A10.8 | |
| 1.21 | A10.9 | |
| 1.22 | A1.11 | PdCl₂(dppf) instead of Pd(PPh₃)₄ |
| 1.23 | A1.12 | PdCl₂(dppf) instead of Pd(PPh₃)₄ |
| 1.24 | A1.13 | PdCl₂(dppf) instead of Pd(PPh₃)₄ |
| 1.25 | A1.14 | PdCl₂(dppf) instead of Pd(PPh₃)₄ |
| 1.26 | A1.16 | PdCl₂(dppf) instead of Pd(PPh₃)₄ |
| 1.27 | A1.15 | PdCl₂(dppf) instead of Pd(PPh₃)₄ |
| 1.28 | A11.8 | PdCl₂(dppf) instead of Pd(PPh₃)₄ |
| 1.29 | A11.1 | PdCl₂(dppf) instead of Pd(PPh₃)₄ |
| 1.30 | A11.11 | PdCl₂(dppf) instead of Pd(PPh₃)₄ |
| 1.31 | A1.17 | PdCl₂(dppf) instead of Pd(PPh₃)₄ |
| 1.32 | A1.18 | PdCl₂(dppf) instead of Pd(PPh₃)₄ |
| 1.33 | A1.20 | PdCl₂(dppf) instead of Pd(PPh₃)₄ |
| 1.34 | A1.19 | PdCl₂(dppf) instead of Pd(PPh₃)₄ |
| 1.35 | A1.21 | PdCl₂(dppf) instead of Pd(PPh₃)₄ |
| 1.36 | A1.22 | PdCl₂(dppf) instead of Pd(PPh₃)₄ |
| 1.37 | A10.10 | |
| 1.38 | A10.11 | |
| 1.39 | A10.30 | PdCl₂(dppf) instead of Pd(PPh₃)₄ |
| 1.40 | A10.12 | |
| 1.41 | A10.13 | |
| 1.42 | A10.7 | |
| 1.43 | A10.14 | PdCl₂(dppf) instead of Pd(PPh₃)₄ |
| 1.44 | A1.38 | |
| 1.45 | A9.7 | |
| 1.46 | A11.5 | PdCl₂(dppf) instead of Pd(PPh₃)₄ |
| 1.47 | A10.15 | |
| 1.48 | A1.29 | |
| 1.49 | A10.17 | |
| 1.50 | A10.18 | |
| 1.51 | A10.19 | |
| 1.52 | A1.8 | |
| 1.53 | A1.37 | |
| 1.54 | A10.31 | |
| 1.55 | A6.1 | PdCl₂(dppf) instead of Pd(PPh₃)₄ |
| 1.56 | A2.1 | PdCl₂(dppf) instead of Pd(PPh₃)₄ |
| 1.57 | A2.2 | PdCl₂(dppf) instead of Pd(PPh₃)₄ |
| 1.58 | A11.7 | PdCl₂(dppf) instead of Pd(PPh₃)₄ |
| 1.59 | A11.10 | PdCl₂(dppf) instead of Pd(PPh₃)₄ |
| 1.60 | A10.20 | PdCl₂(dppf) instead of Pd(PPh₃)₄ |
| 1.61 | A10.21 | PdCl₂(dppf) instead of Pd(PPh₃)₄ |
| 1.62 | A1.35 | PdCl₂(dppf) instead of Pd(PPh₃)₄ |
| 1.63 | A1.36 | PdCl₂(dppf) instead of Pd(PPh₃)₄ |
| 1.64 | A10.2 | B8.2 was used instead of B8.1; |
| | | PdCl₂(dppf) instead of Pd(PPh₃)₄ |
| 1.65 | A9.10 | B8.2 was used instead of B8.1; |
| | | PdCl₂(dppf) instead of Pd(PPh₃)₄ |

### Example 2.1: 4-((1S,2S)-2-(3-amino-4-fluorophenyl)cyclopropyl)-3-chloro-2′-(2-(2-hydroxypropan-2-yl)pyrimidin-4-yl)-5′,6-dimethyl-2H-[1,4′-bipyridin]-2-one

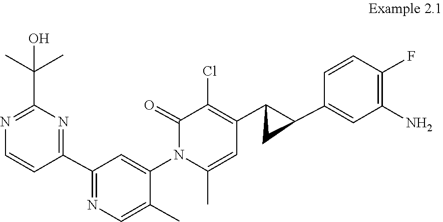 [SMILES: Cc1cnc(-c2ccnc(C(C)(C)O)n2)cc1-n1c(C)cc([C@H]2C[C@@H]2c2ccc(F)c(N)c2)c(Cl)c1=O]

Step 1. A vial was charged with 3-chloro-4-((1S,2S)-2-(3-chloro-4-fluorophenyl)cyclopropyl)-2′-(2-(2-hydroxypropan-2-yl)pyrimidin-4-yl)-5′,6-dimethyl-2H-[1,4′-bipyridin]-2-one (Example 1.11) (60 mg, 0.11 mmol), BocNH₂ (65 mg, 0.56 mmol), BrettPhos Pd G3 (10 mg, 0.011 mmol), Cs₂CO₃ (109 mg, 0.33 mmol) and 1,4-dioxane (2 mL). The mixture was sparged for 2 minutes with argon and then heated to 90° C. for 8 hrs. The mixture was filtered over celite, concentrated, and subjected to flash chromatography (SiO₂, hexanes-EtOAc) to give tert-butyl (5-((1S,2S)-2-(3-chloro-2′-(2-(2-hydroxypropan-2-yl)pyrimidin-4-yl)-5′,6-dimethyl-2-oxo-2H-[1,4′-bipyridin]-4-yl)cyclopropyl)-2-fluorophenyl)carbamate.

Step 2. To tert-butyl (5-((1S,2S)-2-(3-chloro-2′-(2-(2-hydroxypropan-2-yl)pyrimidin-4-yl)-5′,6-dimethyl-2-oxo-2H-[1,4′-bipyridin]-4-yl)cyclopropyl)-2-fluorophenyl)carbamate was added DCM (1 mL) and TFA (1 mL). The mixture was stirred for 15 min, concentrated, and subjected to reverse phase HPLC (Silicycle SiliaChrom SB C18, 5 μm, 150 Å, 30×250 mm; 0.1% TFA in MeCN-0.1% TFA in water) to give 4-((1S,2S)-2-(3-amino-4-fluorophenyl)cyclopropyl)-3-chloro-2′-(2-(2-hydroxypropan-2-yl)pyrimidin-4-yl)-5′,6-dimethyl-2H-[1,4′-bipyridin]-2-one (Example 2.1).

Example 2.2: 4-((1S,2S)-2-(3-amino-4-fluorophenyl)cyclopropyl)-3-chloro-2′-(2-(2-hydroxypropan-2-yl)pyrimidin-4-yl)-5′,6-dimethyl-2H-[1,4′-bipyridin]-2-one Example 2.2 was prepared analogously to step 1 in the preparation of Example 2.1 using acetamide instead of BocNH₂. Individual atropisomers could be separated on reverse phase HPLC.

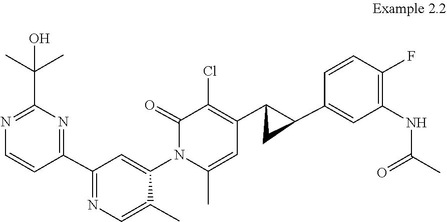

### Example 2.3: N-(5-((1S,2S)-2-(3-chloro-2′-(2-(2-hydroxypropan-2-yl)pyrimidin-4-yl)-5′,6-dimethyl-2-oxo-2H-[1,4′-bipyridin]-4-yl)cyclopropyl)-2-fluorophenyl)cyclopropanecarboxamide

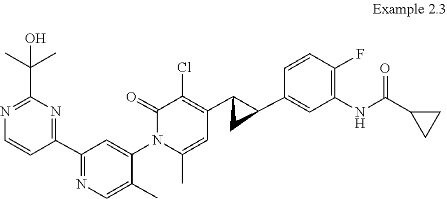 [SMILES: Cc1cnc(-c2ccnc(C(C)(C)O)n2)cc1-n1c(C)cc([C@H]2C[C@@H]2c2ccc(F)c(NC(=O)C3CC3)c2)c(Cl)c1=O]

A vial was charged with 4-((1S,2S)-2-(3-amino-4-fluorophenyl)cyclopropyl)-3-chloro-2′-(2-(2-hydroxypropan-2-yl)pyrimidin-4-yl)-5′,6-dimethyl-2H-[1,4′-bipyridin]-2-one (4.0 mg, 0.0077 mmol), pyridine (5 μL), and DCM (0.5 mL), followed by cyclopropanecarbonyl chloride (0.8 mg, 00077 mmol). The mixture was allowed to stir at room temperature for 10 minutes, was concentrated and subjected to reverse phase HPLC (Silicycle SiliaChrom SB C18, 5 μm, 150 Å, 30×250 mm; 0.1% TFA in MeCN-0.10% TFA in water) to give N-(5-((1 S,2S)-2-(3-chloro-2′-(2-(2-hydroxypropan-2-yl)pyrimidin-4-yl)-5′,6-dimethyl-2-oxo-2H-[1,4′-bipyridin]-4-yl)cyclopropyl)-2-fluorophenyl)cyclopropanecarboxamide (Example 2.3).

### Example 3.1: 3-(3-chloro-4-((1S,2S)-2-(4-fluorophenyl)cyclopropyl)-5′,6-dimethyl-2-oxo-2H-[1,4′-bipyridin]-2′-yl)-2-fluoro-N,N-dimethylbenzamide

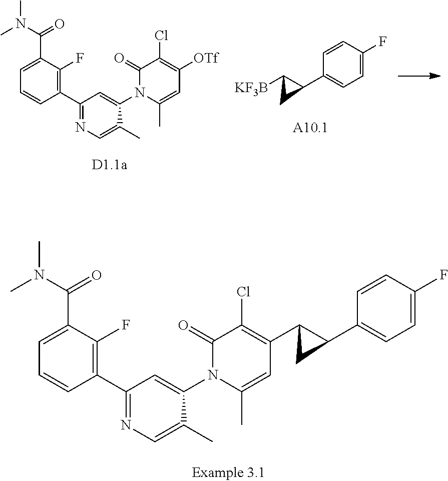

A vial was charged with 3-chloro-2′-(3-(dimethylcarbamoyl)-2-fluorophenyl)-5′,6-dimethyl-2-oxo-2H-[1,4′-bipyridin]-4-yl trifluoromethanesulfonate (D1.1a)(26 mg, 0.047 mmol), trifluoro((1 S,2S)-2-(4-fluorophenyl)cyclopropyl)-λ⁴-borane, potassium salt (14 mg, 0.056 mmol), Cs₂CO₃ (46 mg, 0.14 mmol), Pd(dppf)Cl₂-DCM (3.8 mg, 0.0047 mmol), PhMe (0.5 mL), and water (0.1 mL). The mixture was heated to 80° C. for 1 hr, before being cooled to room temperature. The mixture as passed through a plug of celite, washing with EtOAc, and concentrated. The residue was subjected to reverse phase HPLC (Silicycle SiliaChrom SB C18, 5 μm, 150 Å, 30×250 mm; 0.1% TFA in MeCN-0.1% TFA in water) to afford 4-(3-chloro-4-((1S,2S)-2-(4-fluorophenyl)cyclopropyl)-5′,6-dimethyl-2-oxo-2H-[1,4′-bipyridin]-2′-yl)-N,N-dimethylpyrimidine-2-carboxamide (Example 3.1).

The following compounds in Table 2 were prepared analogously to Example 3.1 modified as described:

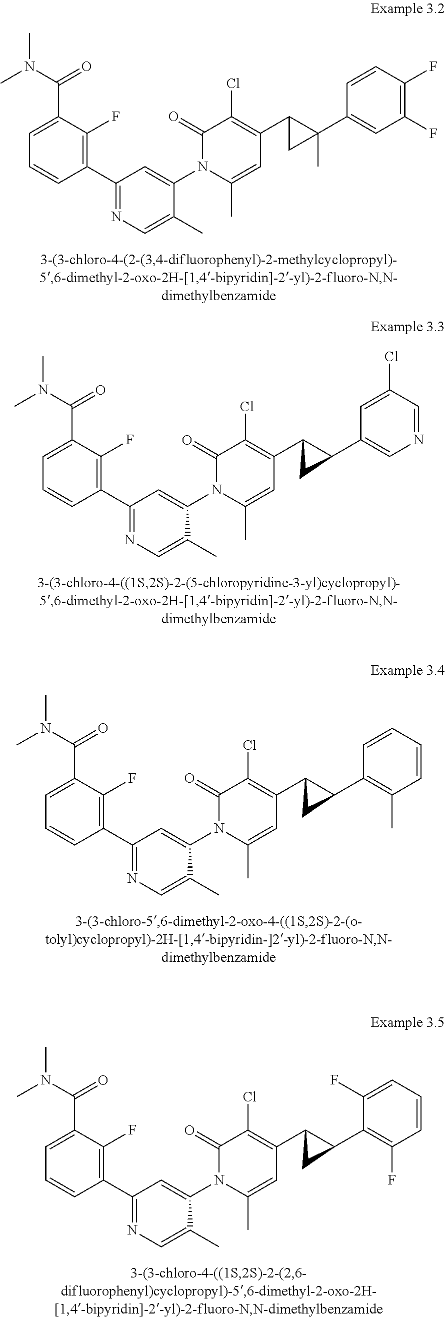

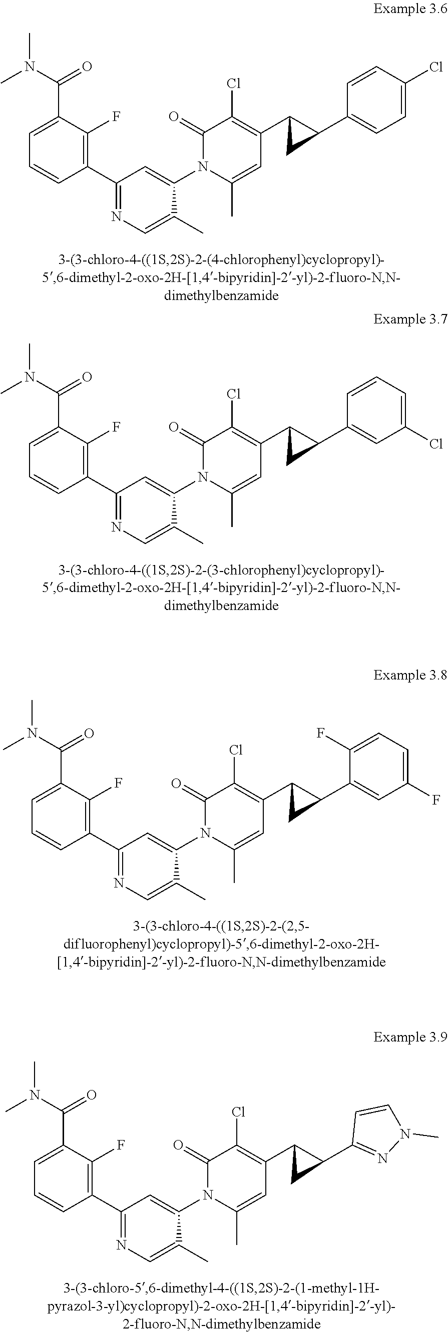

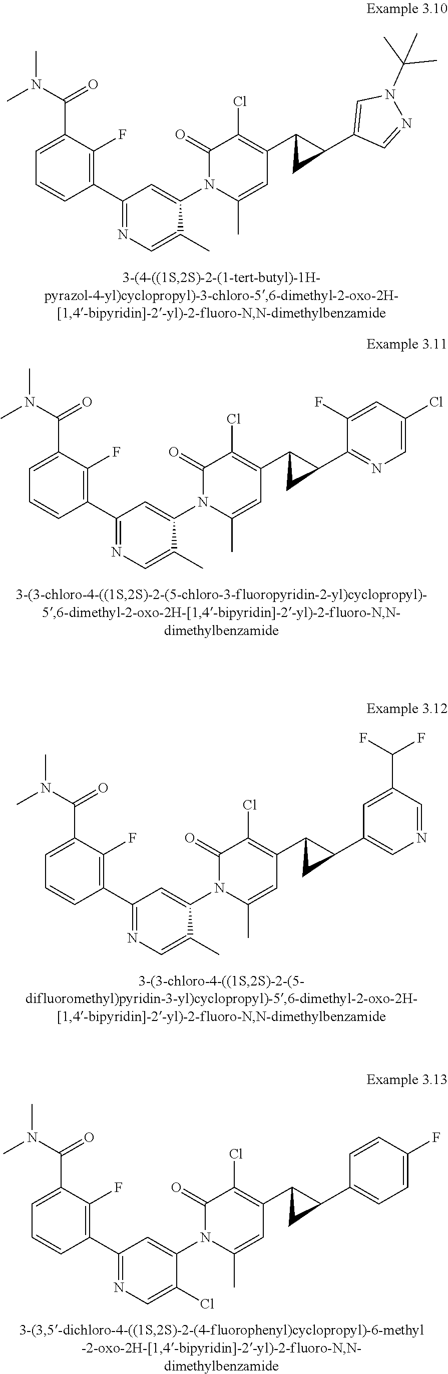

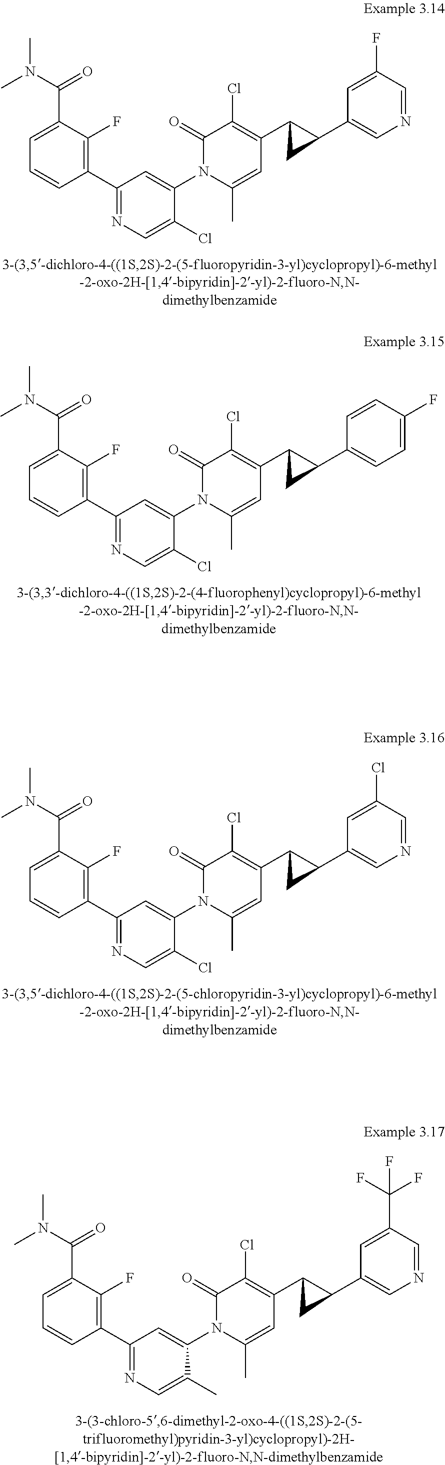

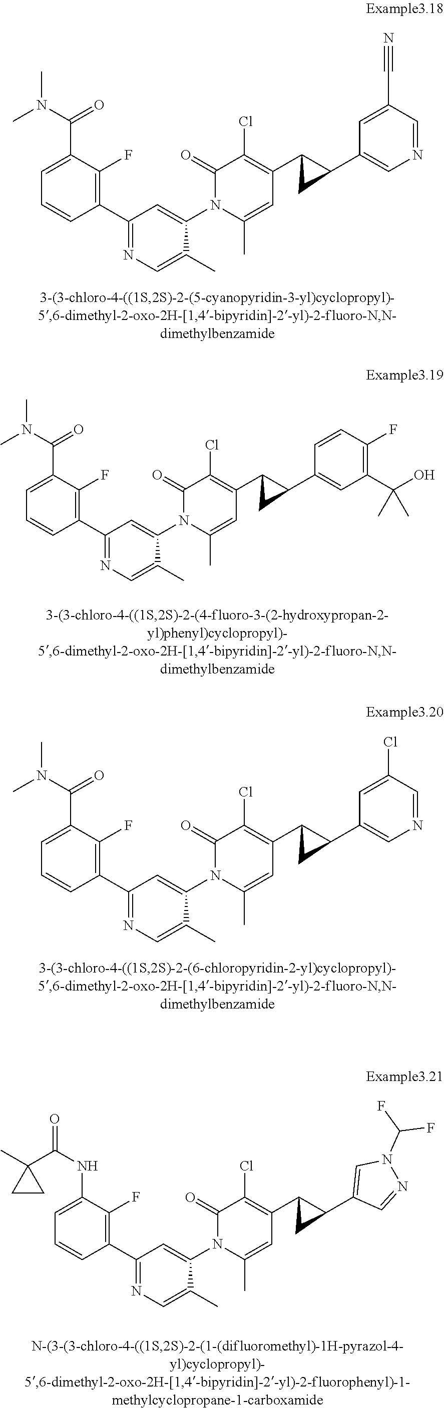

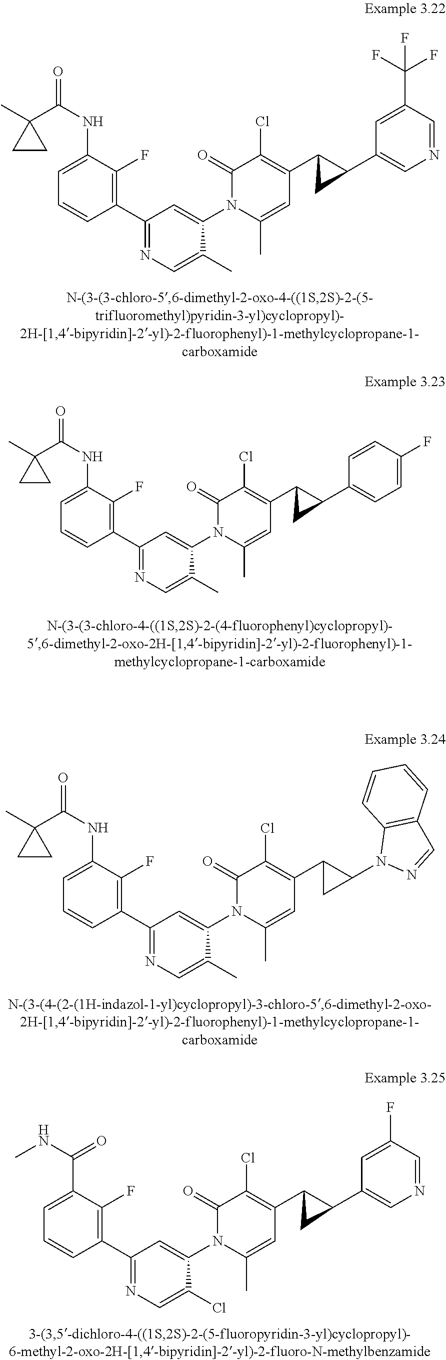

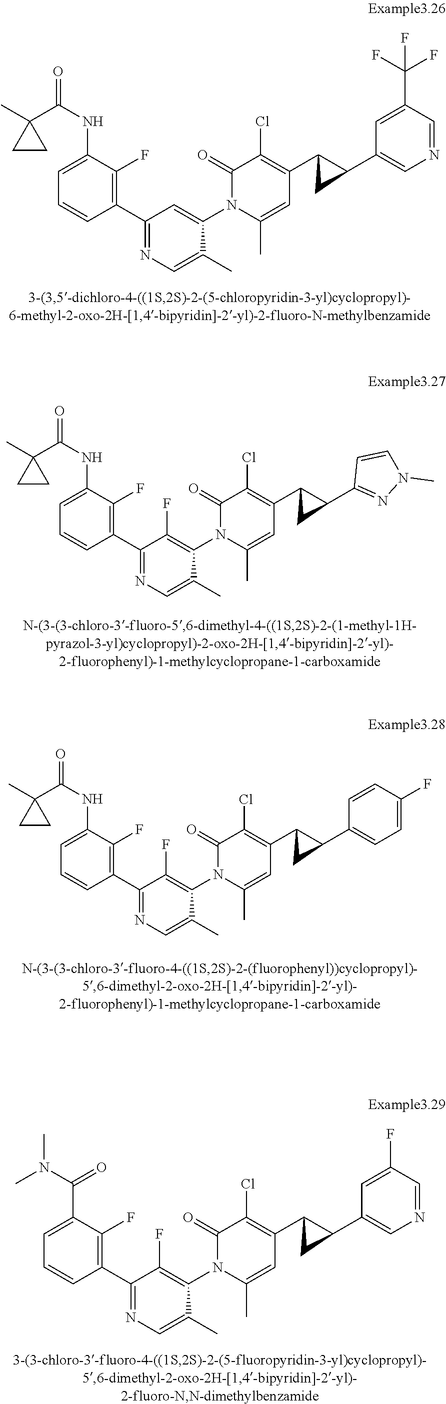

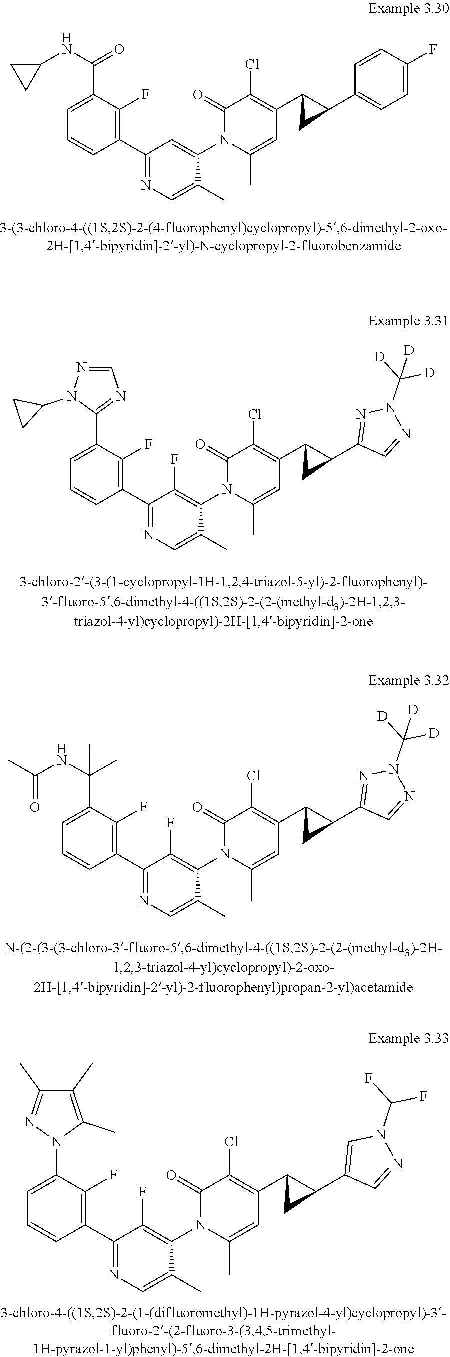

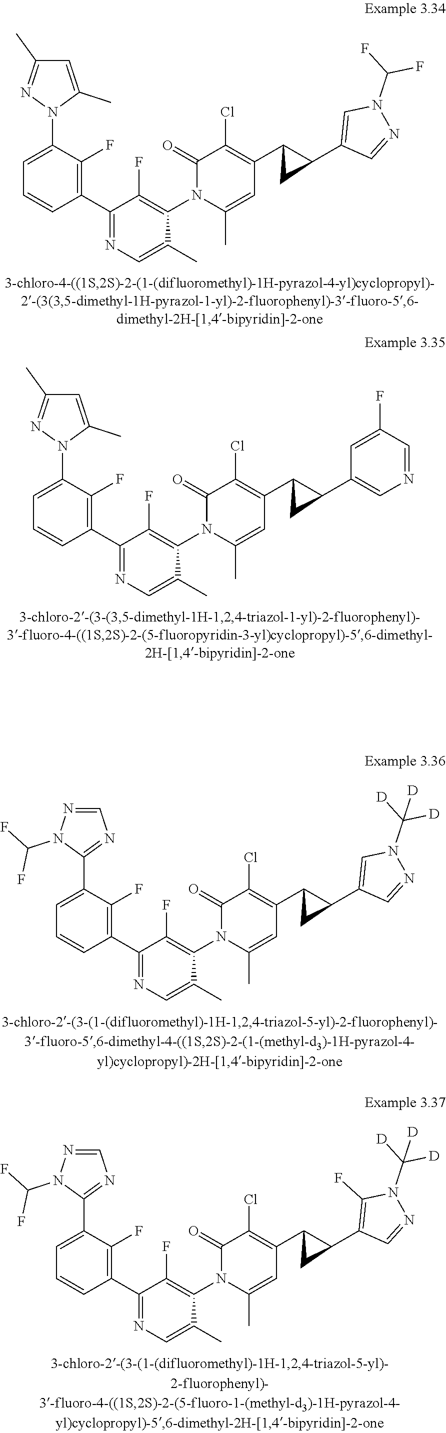

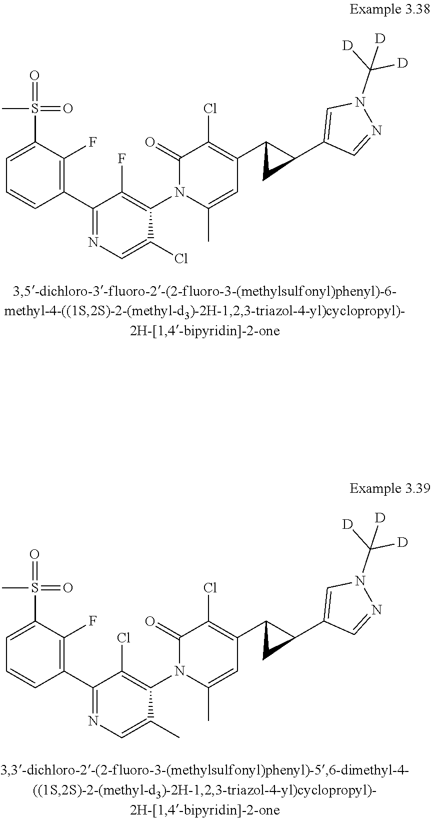

TABLE 2
Procedures analogous to Example 3.1 Synthesis

| Example | Instead of | Variations from |
|---|---|---|
| Number | A10.1 | Example 3.1 Synthesis |
| 3.2 | A10.26 | D2.1 used instead of D1.1a, |
| | | Pd(PPh₃)₄ was used instead |
| | | of Pd(dppf)Cl₂ |
| 3.3 | A9.1 | |
| 3.4 | A11.9 | |
| 3.5 | A11.13 | |
| 3.6 | A11.15 | |
| 3.7 | A11.14 | |
| 3.8 | A11.4 | |
| 3.9 | A11.6 | |
| 3.10 | A7.4 | |
| 3.11 | A11.2 | |
| 3.12 | A9.6 | |
| 3.13 | A10.1 | Used D1.2 instead of D1.1a |
| 3.14 | A10.32 | Used D1.2 instead of D1.1a |
| 3.15 | A10.1 | Used D1.3 instead of D1.1a |
| 3.16 | A9.1 | Used D1.2 instead of D1.1a |
| 3.17 | A1.19 | |
| 3.18 | A1.18 | |
| 3.19 | A7.3 | |
| 3.20 | A7.2 | |
| 3.21 | A9.7 | Used D1.5 instead of D1.1a |
| 3.22 | A1.19 | Used D1.5 instead of D1.1a |
| 3.23 | A10.1 | Used D1.5 instead of D1.1a |
| 3.24 | A10.33 | Used D1.5 instead of D1.1a |
| 3.25 | A10.32 | Used D1.4 instead of D1.1a |
| 3.26 | A10.34 | Used D1.4 instead of D1.1a |
| 3.27 | A11.6 | Used D1.6 instead of D1.1a |
| 3.28 | A10.1 | Used D1.6 instead of D1.1a |
| 3.29 | A10.32 | Used D1.8 instead of D1.1a |
| 3.30 | A10.1 | Used D1.7 instead of D1.1a |
| 3.31 | A9.10 | Used D1.9 instead of D1.1a |
| 3.32 | A9.10 | Used D1.14 instead of D1.1a |
| 3.33 | A9.7 | Used D1.13 instead of D1.1a |
| 3.34 | A9.7 | Used D1.12 instead of D1.1a |
| 3.35 | A10.32 | Used D1.11 instead of D1.1a |
| 3.36 | A9.11 | Used D1.10 instead of D1.1a |
| 3.37 | A9.16 | Used D1.10 instead of D1.1a |
| 3.38 | A9.10 | Used D1.15 instead of D1.1a |
| 3.39 | A9.10 | Used D1.16 instead of D1.1a |

### Example 4.1: N-(3-(3-chloro-4-((1S,2S)-2-(5,6-dihydro-4H-pyrrolo[1,2-b]pyrazol-3-yl)cyclopropyl)-5′,6-dimethyl-2-oxo-2H-[1,4′-bipyridin]-2′-yl)-2-fluorophenyl)-1-methylcyclopropane-1-carboxamide

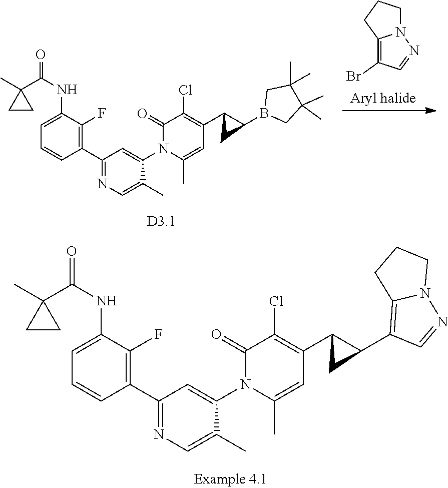

A vial was charged with N-(3-(3-chloro-5′,6-dimethyl-2-oxo-4-((1S,2S)-2-(4,4,5,5-tetramethyl-1,3,2-dioxaborolan-2-yl)cyclopropyl)-2H-[1,4′-bipyridin]-2′-yl)-2-fluorophenyl)-1-methylcyclopropane-1-carboxamide (D3.1) (30 mg, 0.053 mmol), 3-bromo-5,6-dihydro-4H-pyrrolo[1,2-b]pyrazole (15 mg, 0.080 mmol), cataXCiumA Pd G3 (3.8 mg, 0.0053 mmol), cataXCiumA (1.9 mg, 0.0053 mmol), K₃PO₄ (34 mg, 0.16 mmol), water (0.2 mL), and PhMe (1 mL). The mixture was sparged with argon for 2 min, then heated to 90° C. for 1.5 hrs. The mixture was cooled to room temperature, concentrated, and subjected to flash column chromatography (SiO₂, hexanes-EtOAc-MeOH) followed by reverse phase HPLC (Silicycle SiliaChrom SB C18, 5 μm, 150 Å, 30×250 mm; 0.1% TFA in MeCN-0.1% TFA in water) to give N-(3-(3-chloro-4-((1S,2S)-2-(5,6-dihydro-4H-pyrrolo[1,2-b]pyrazol-3-yl)cyclopropyl)-5′,6-dimethyl-2-oxo-2H-[1,4′-bipyridin]-2′-yl)-2-fluorophenyl)-1-methylcyclopropane-1-carboxamide (Example 4.1).

The following compounds in Table 3 were prepared analogously to Example 4.1 modified as

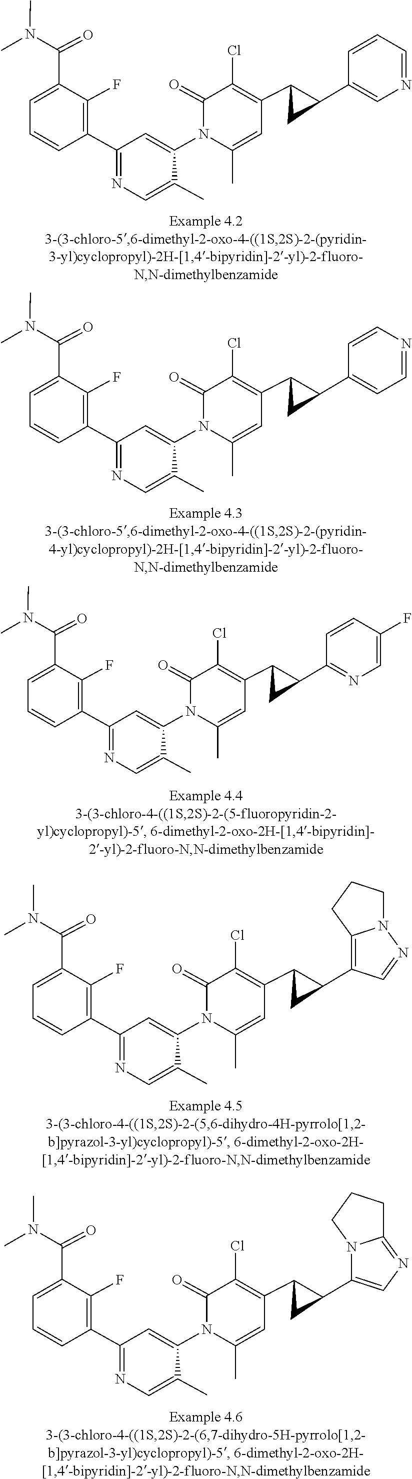

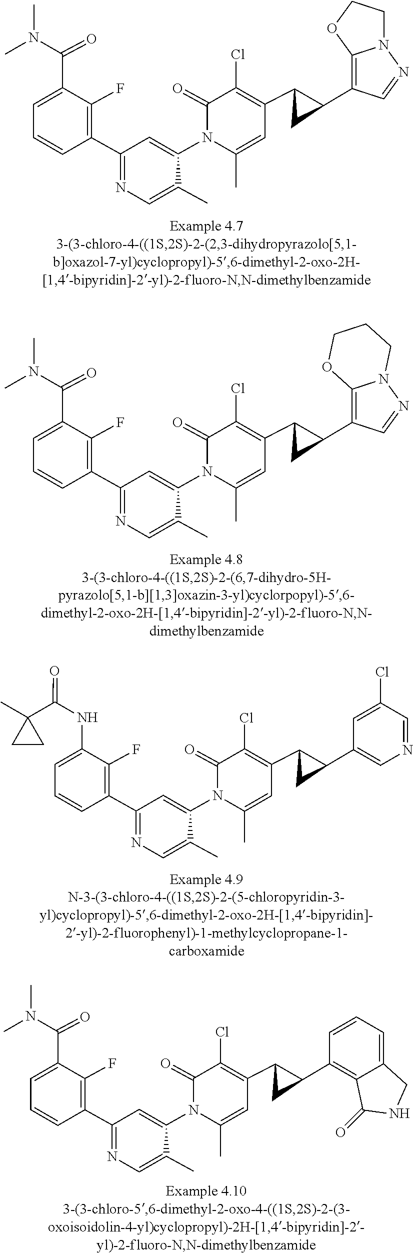

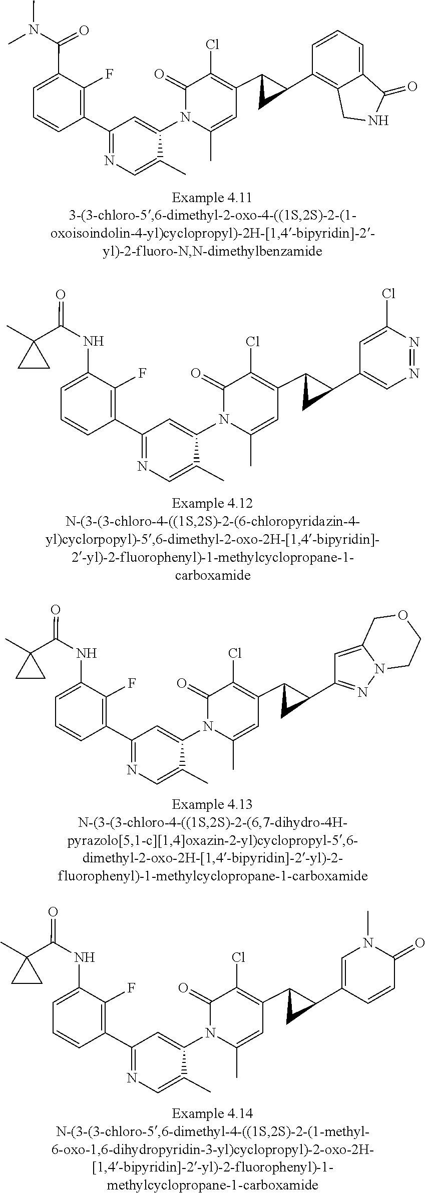

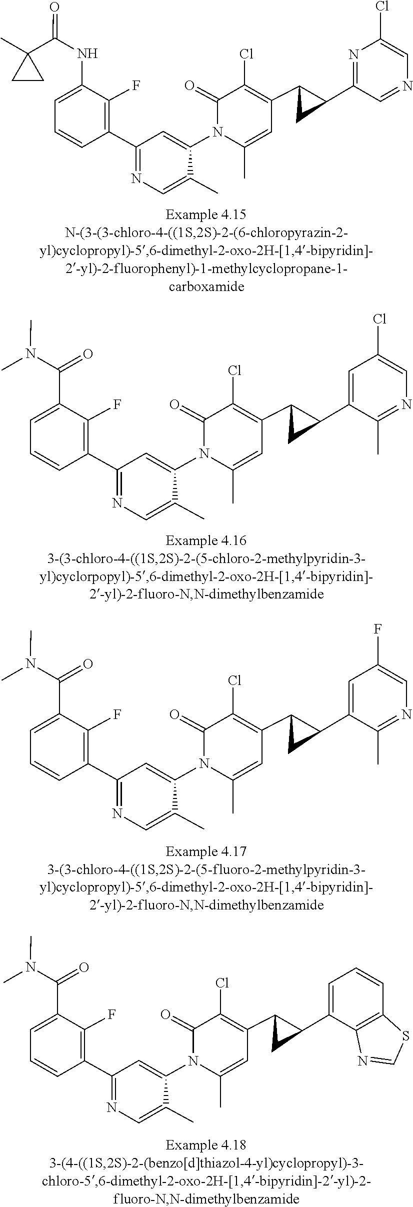

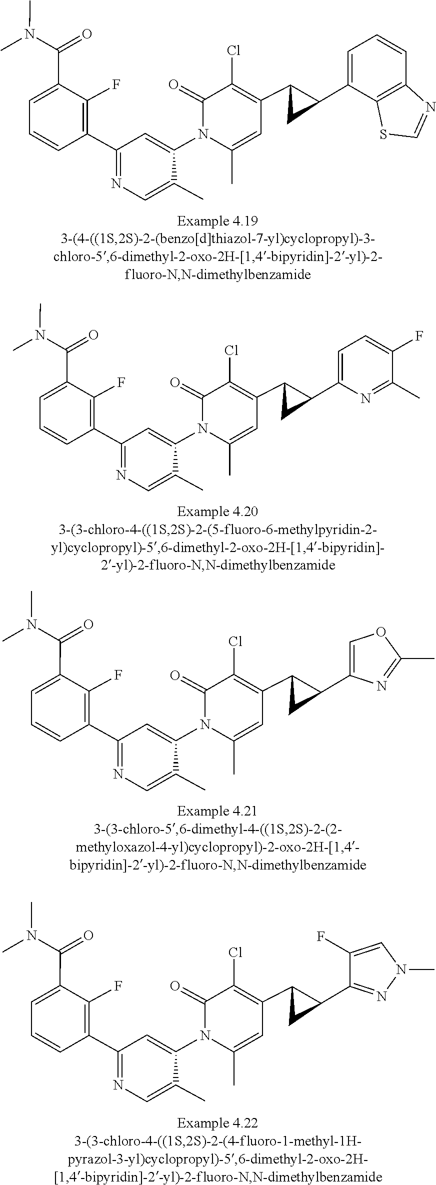

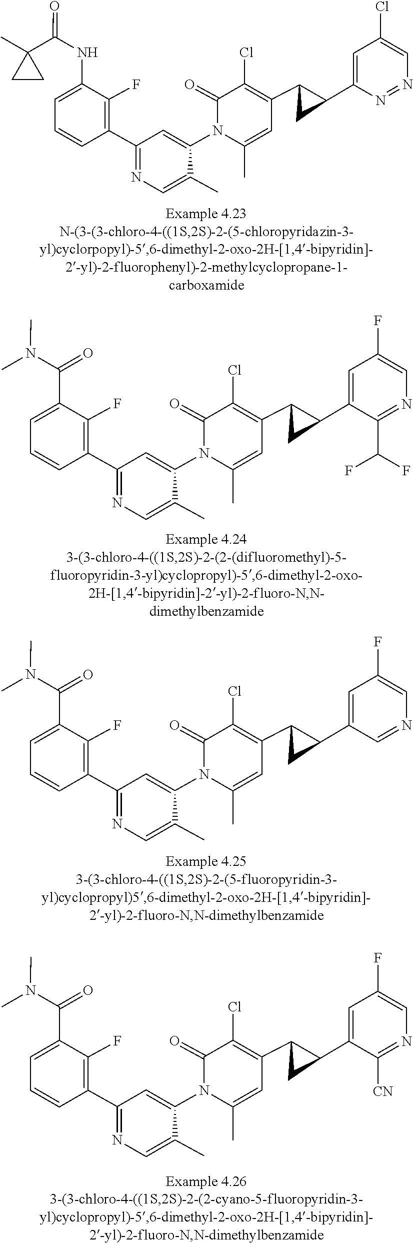

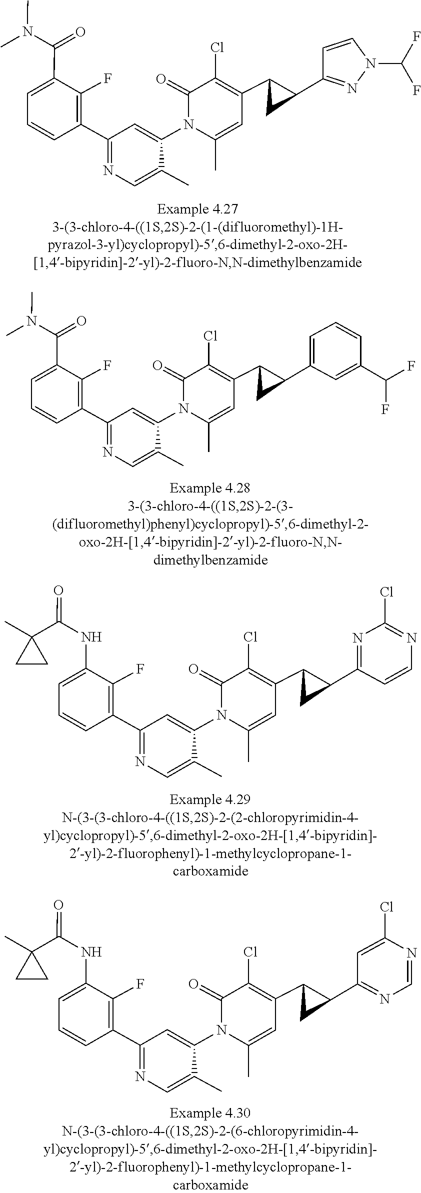

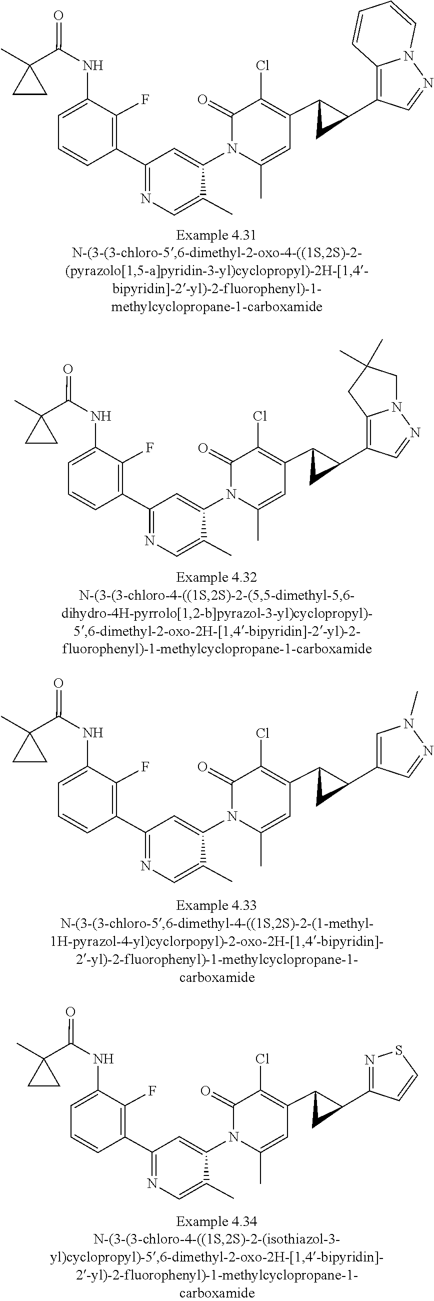

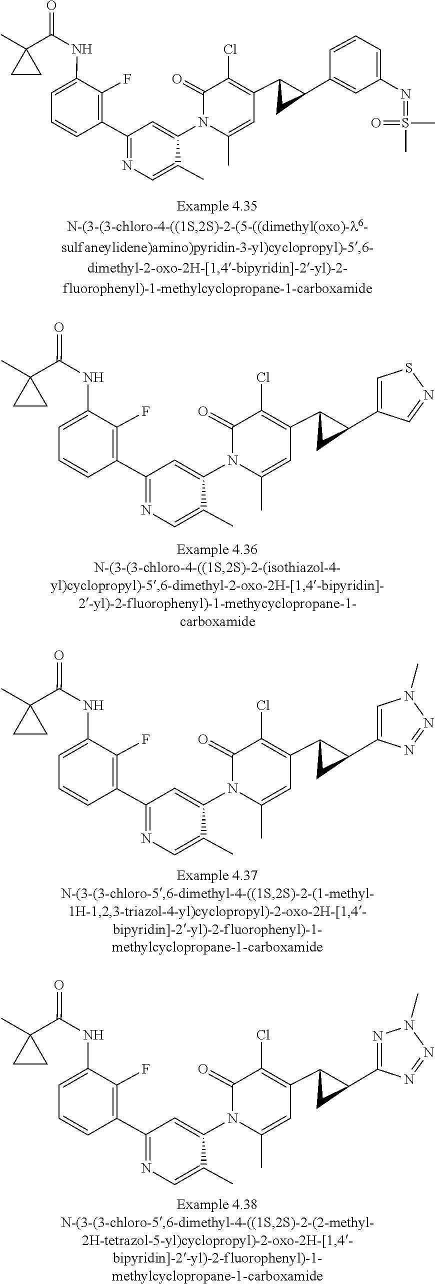

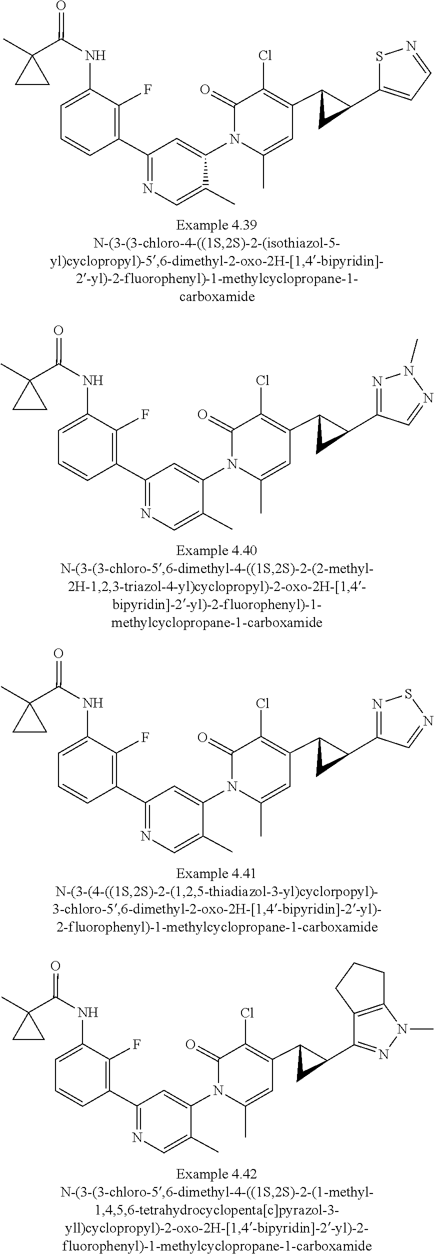

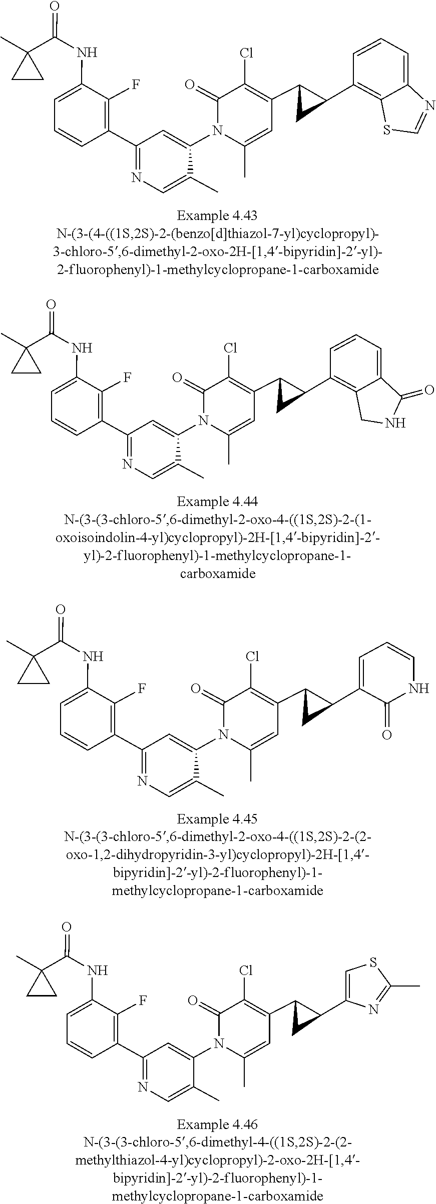

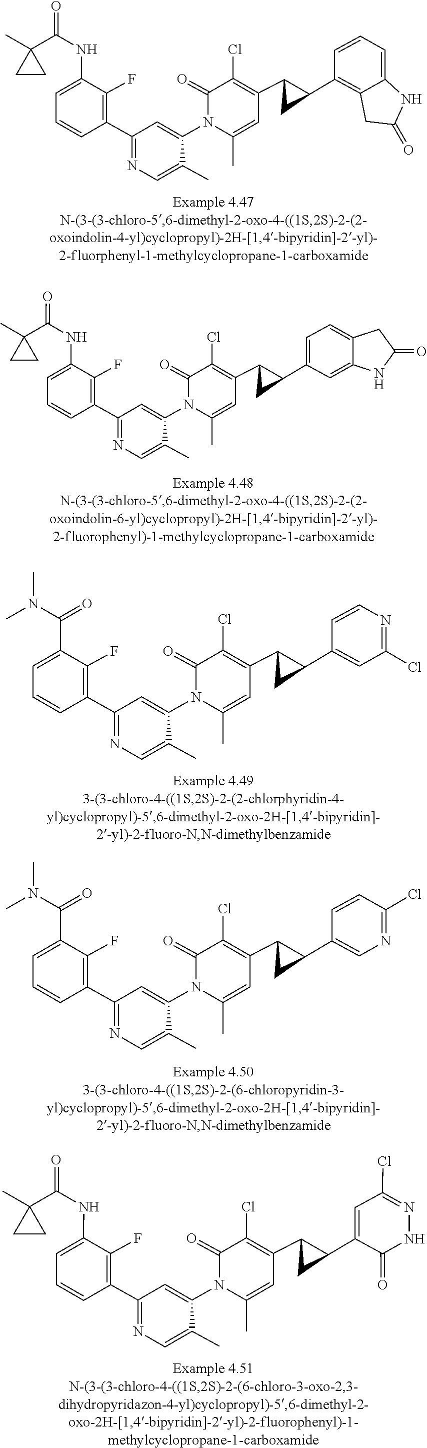

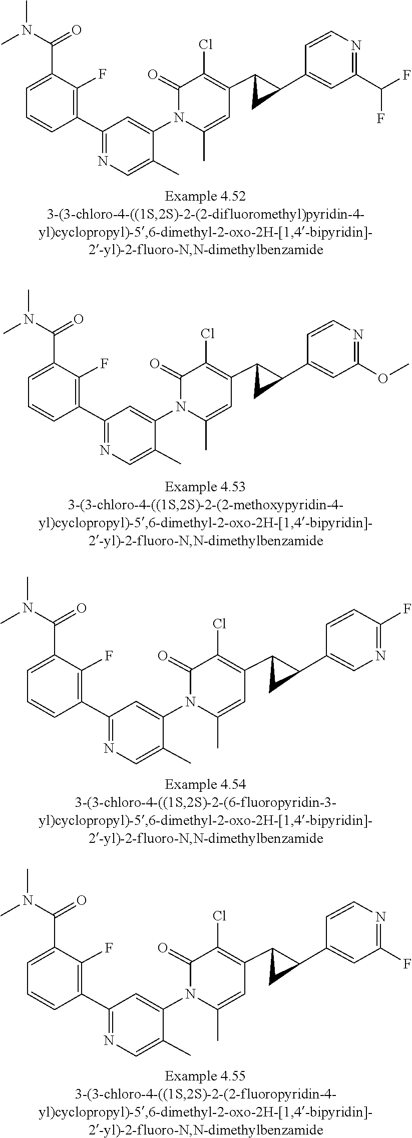

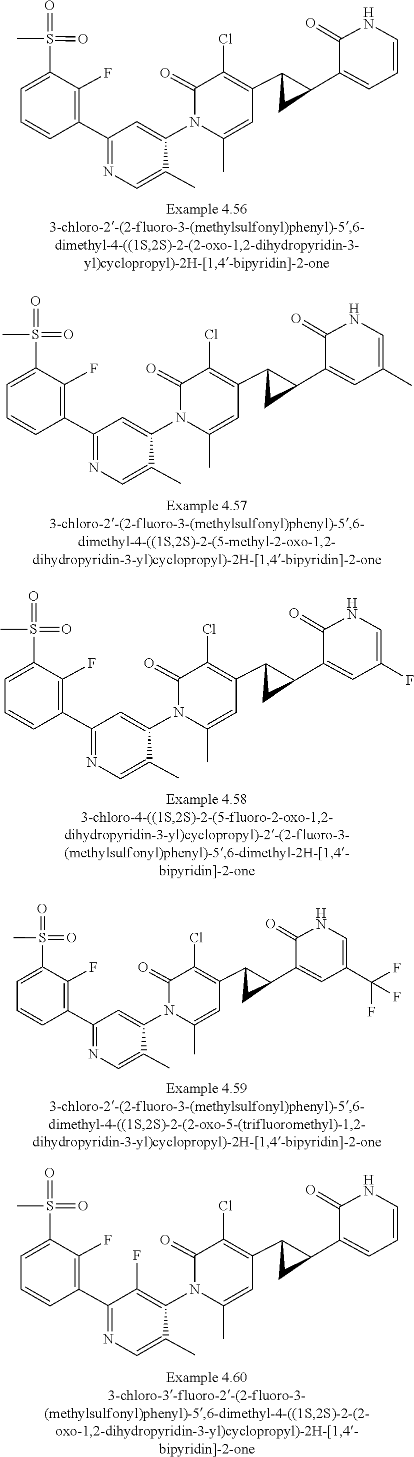

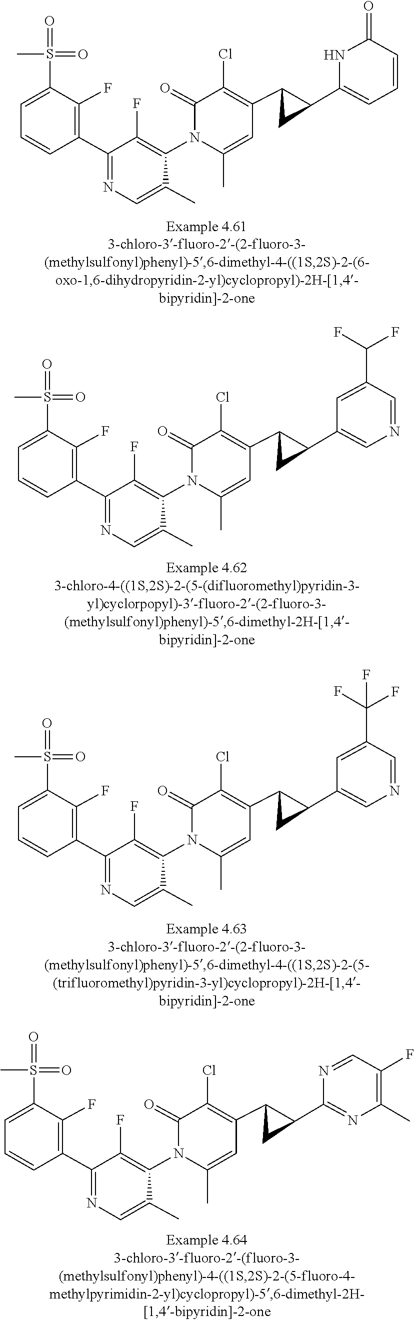

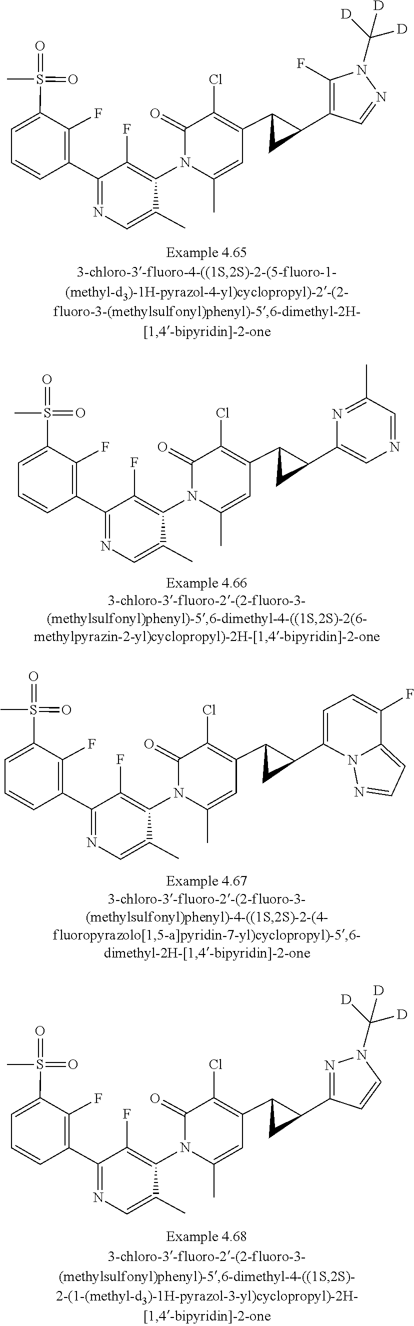

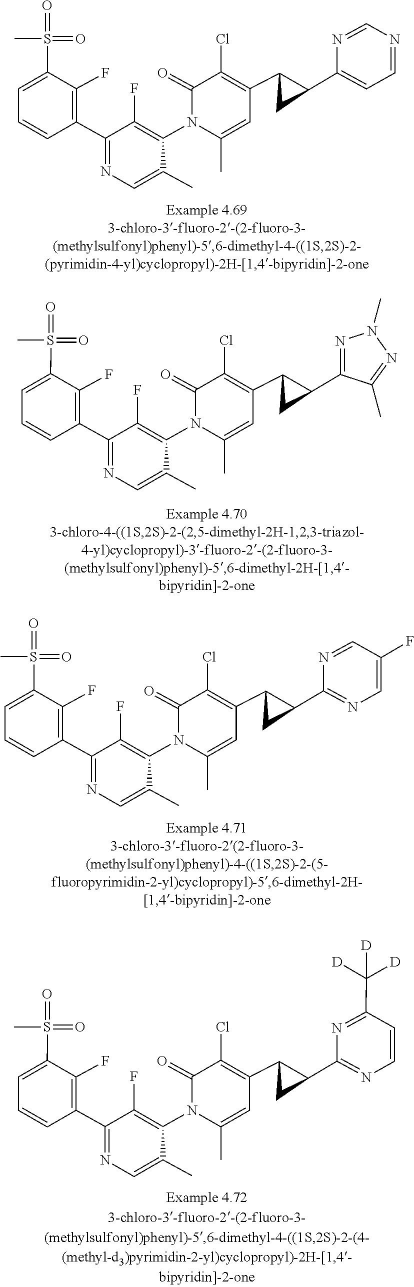

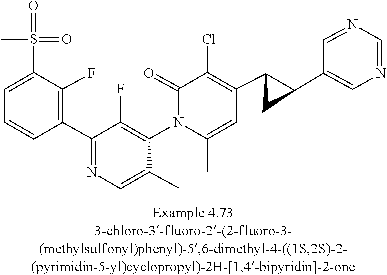

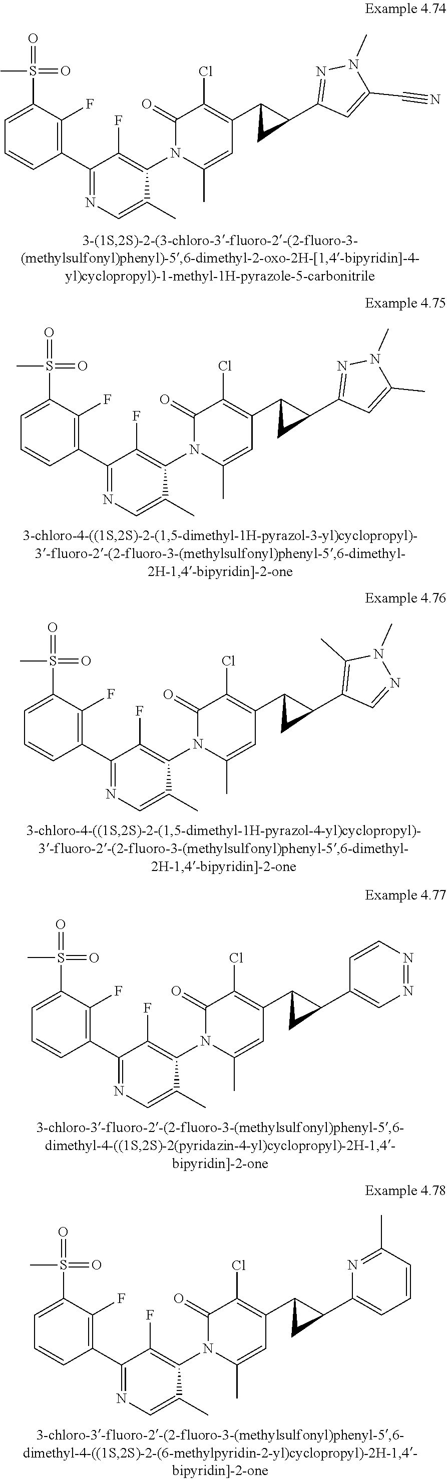

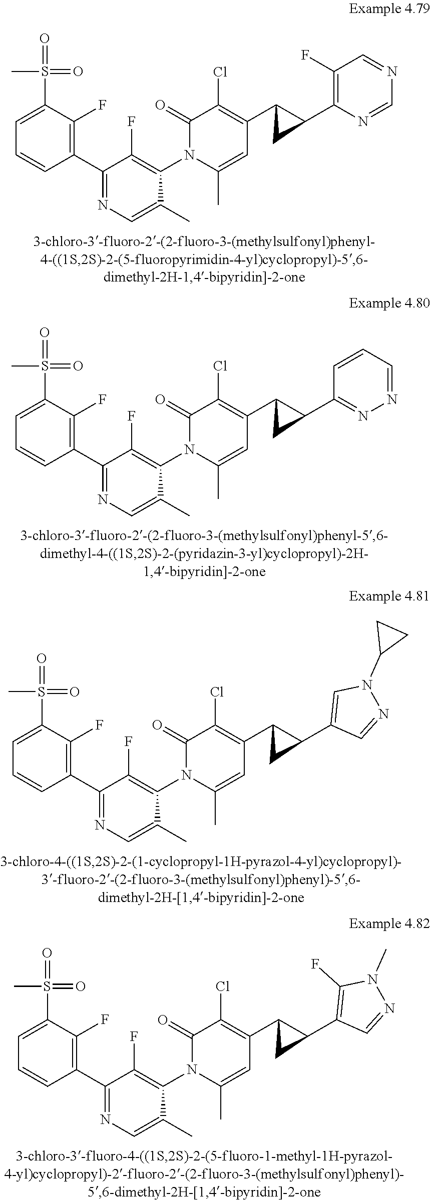

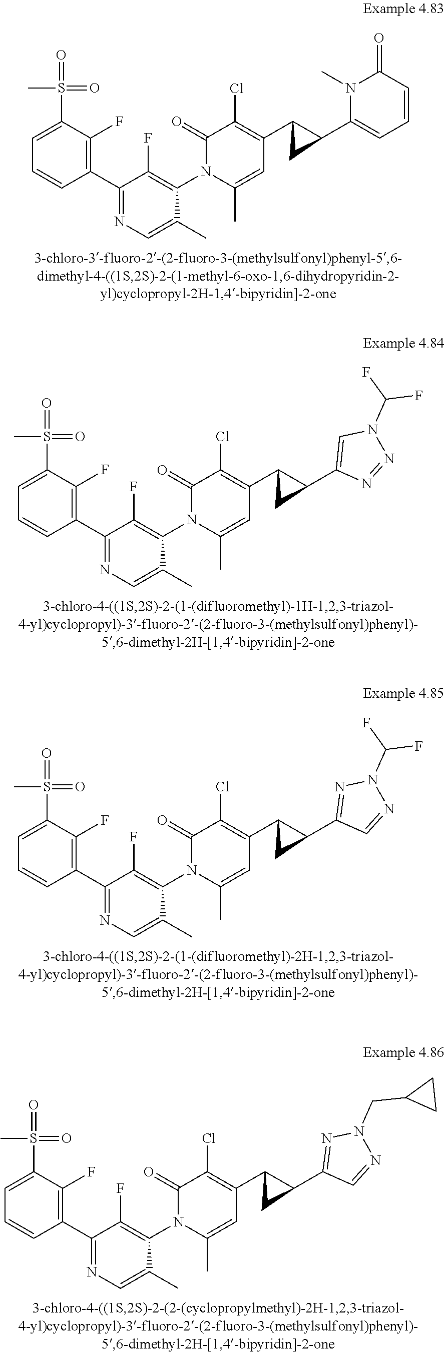

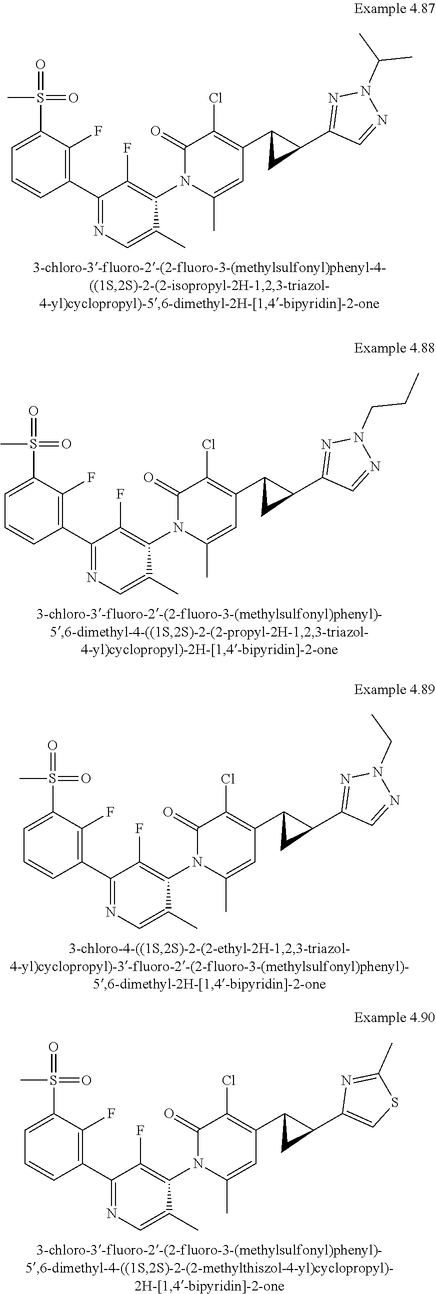

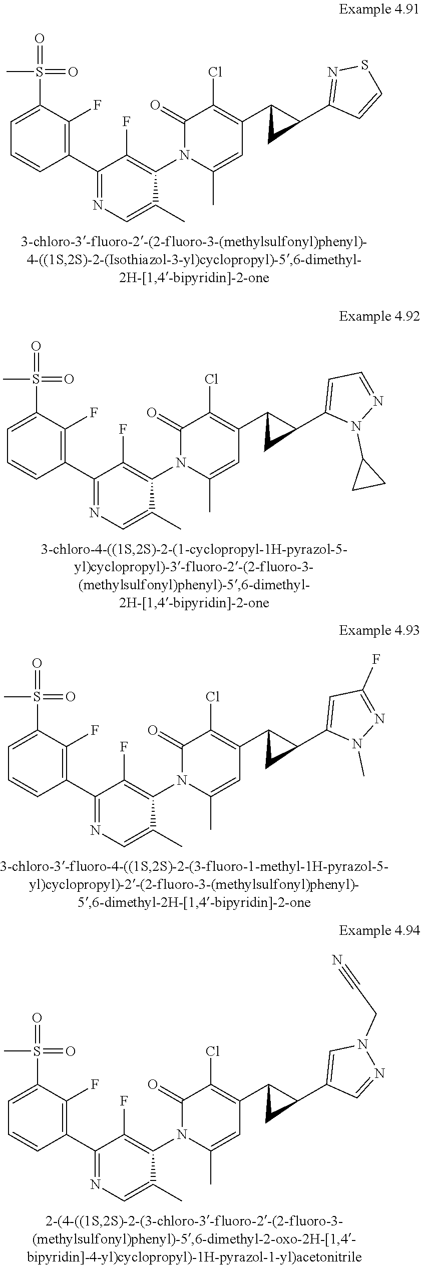

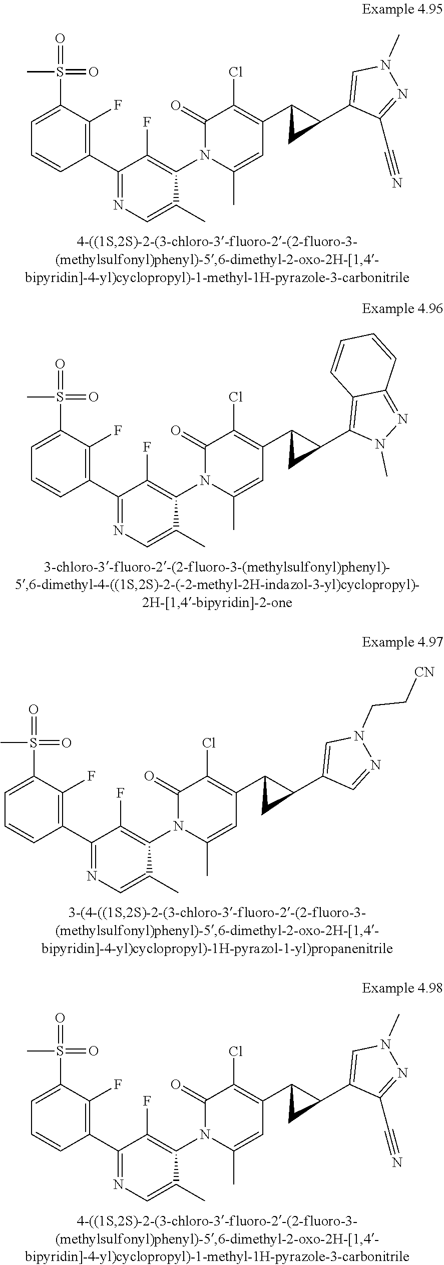

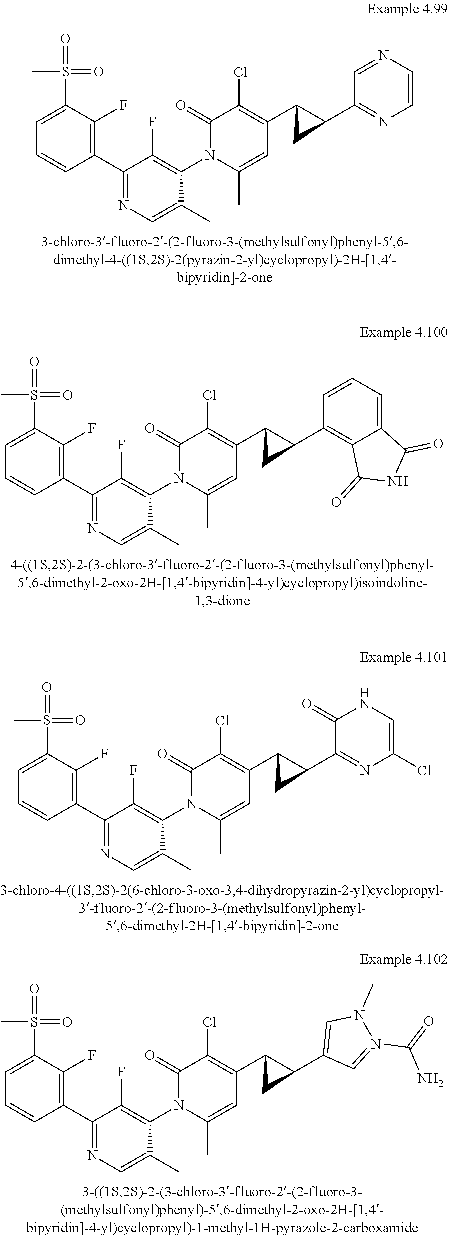

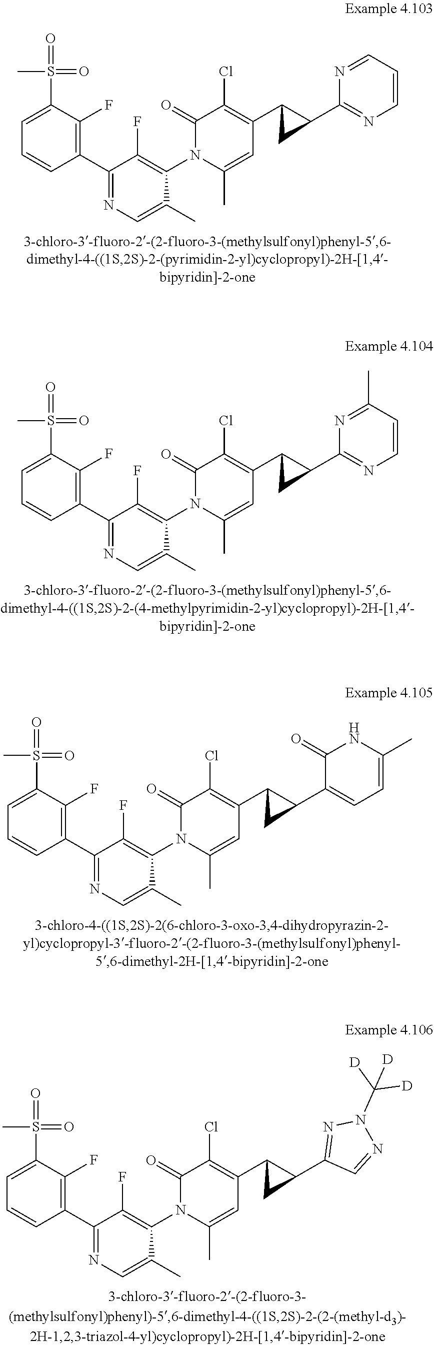

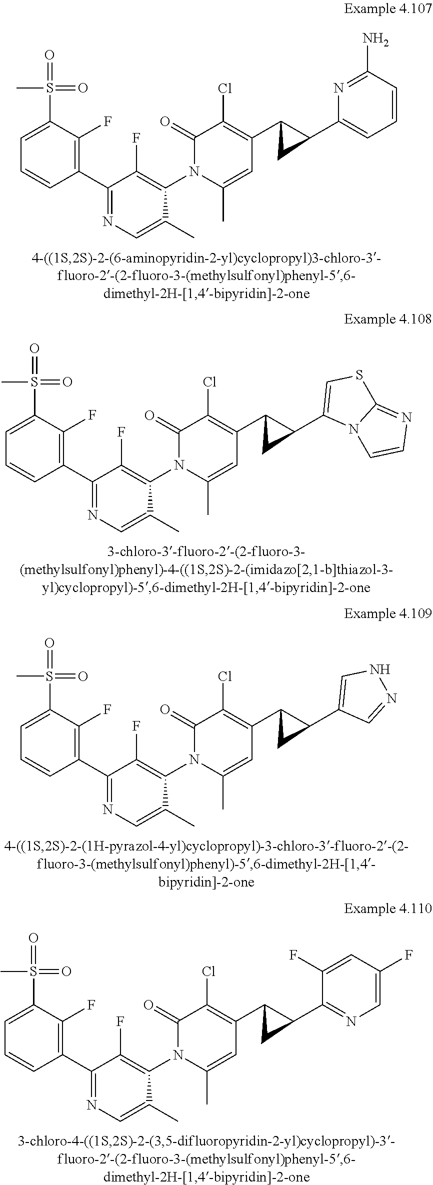

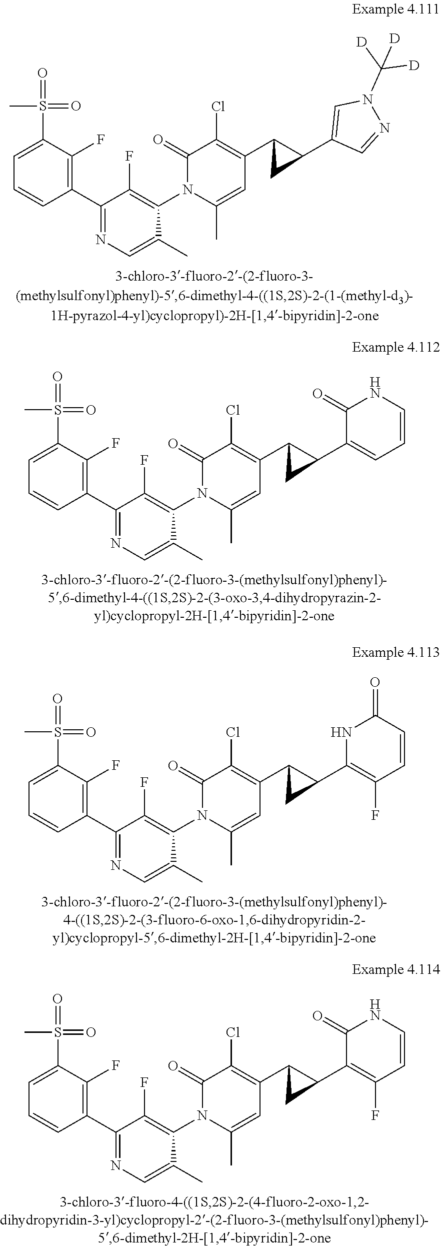

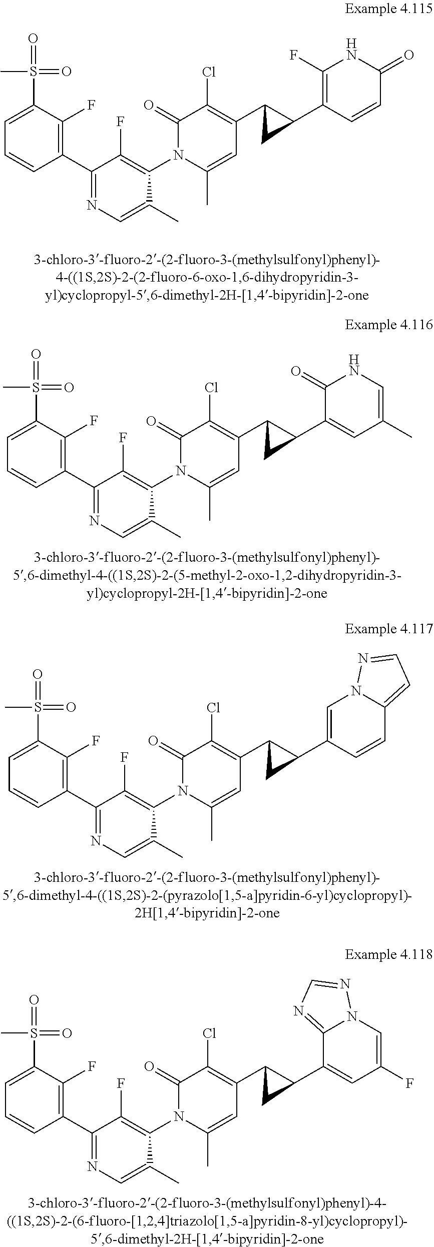

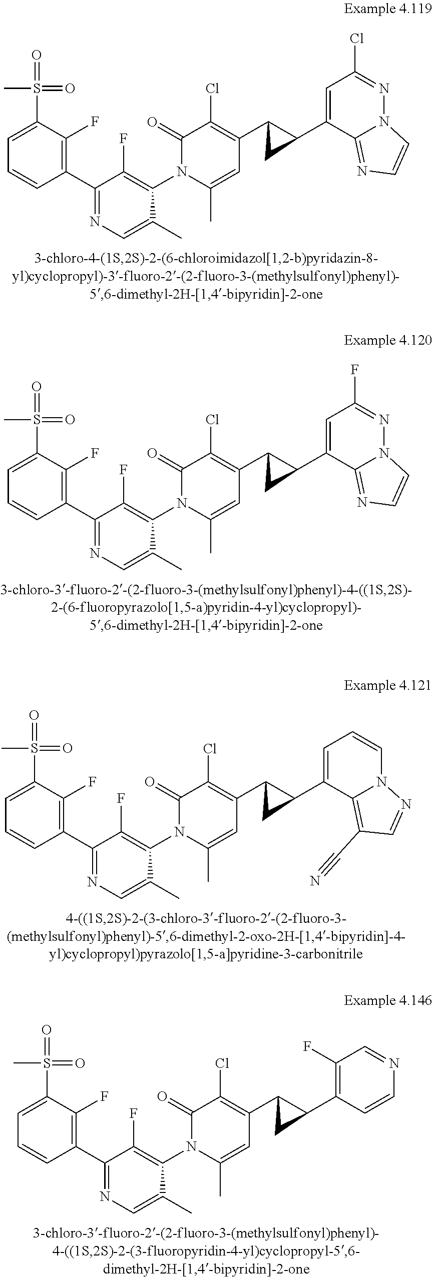

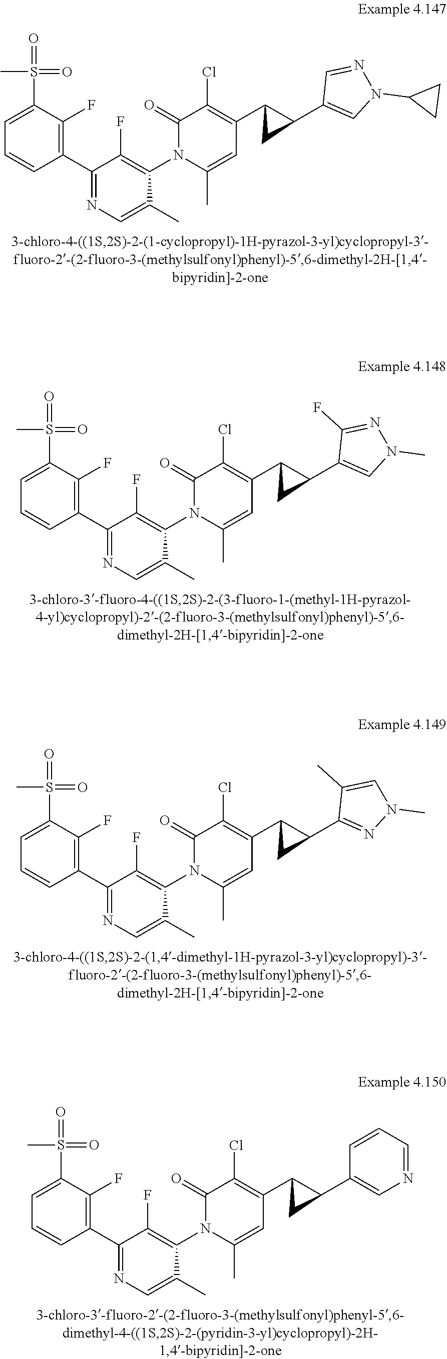

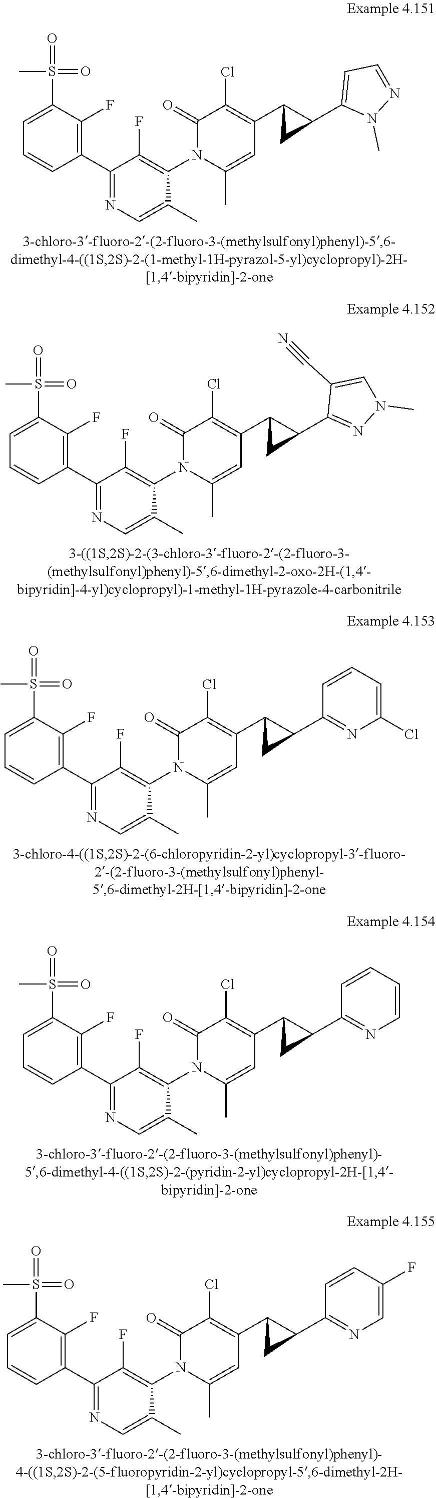

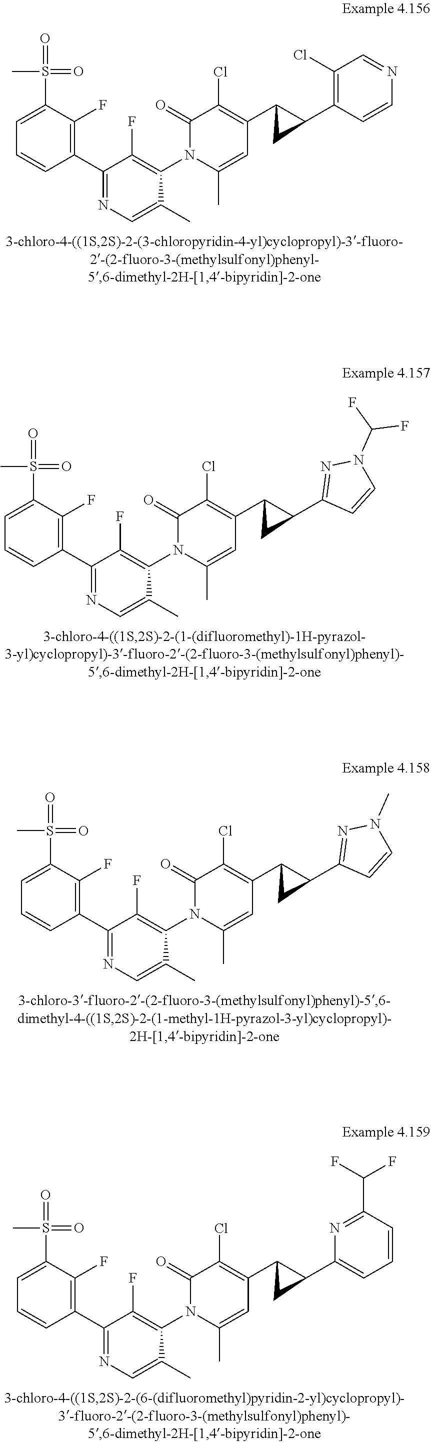

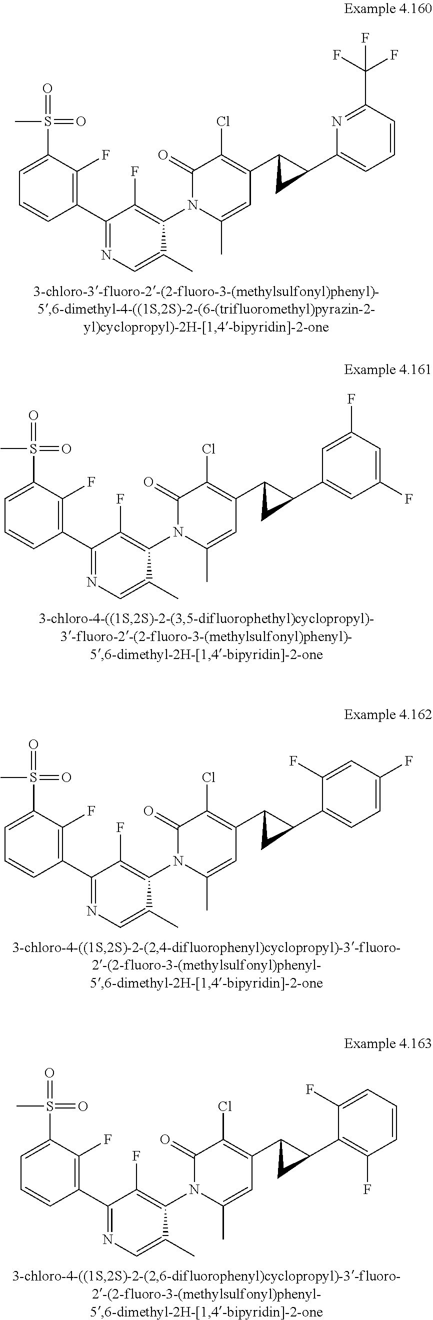

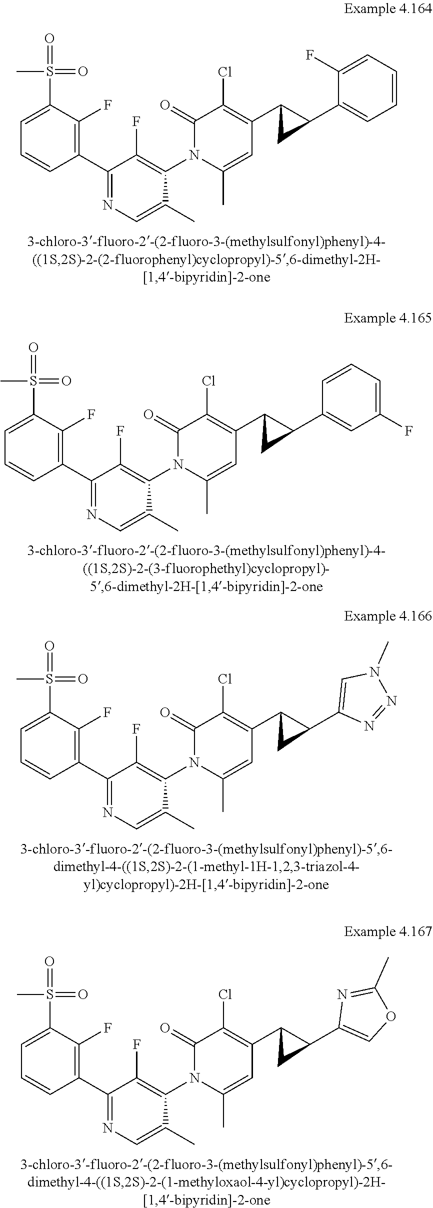

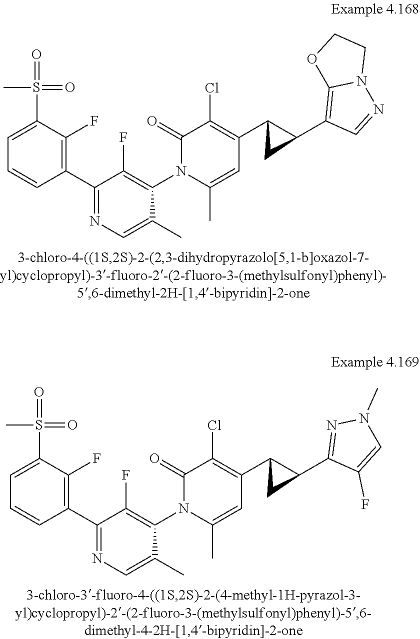

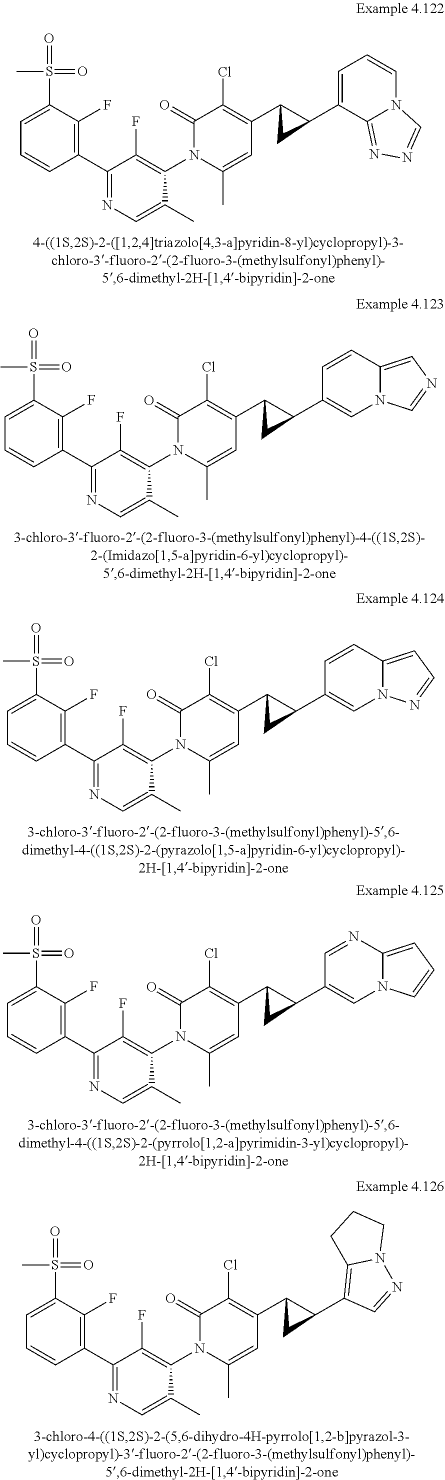

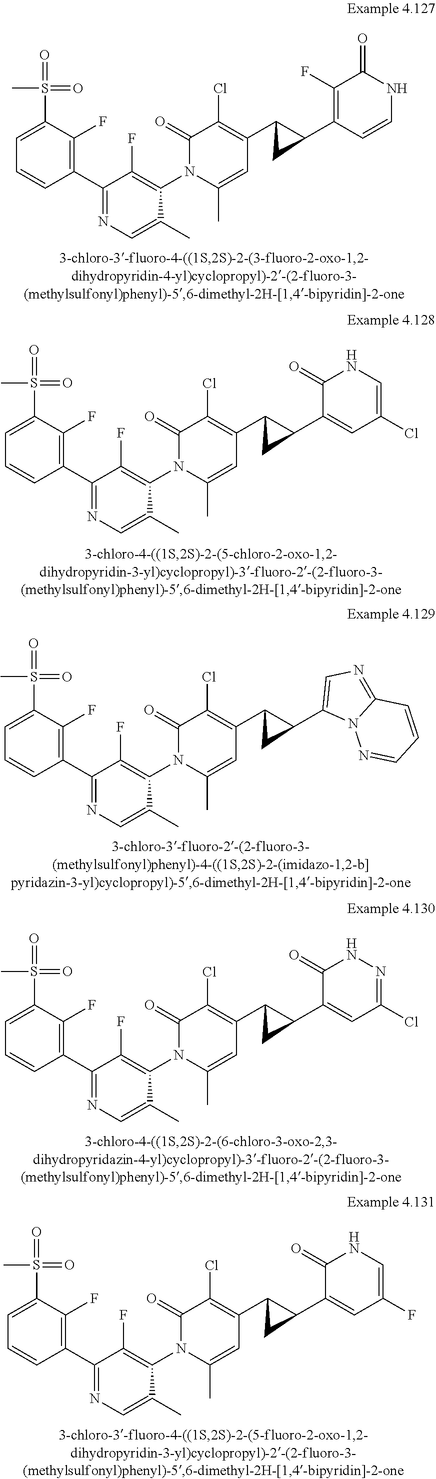

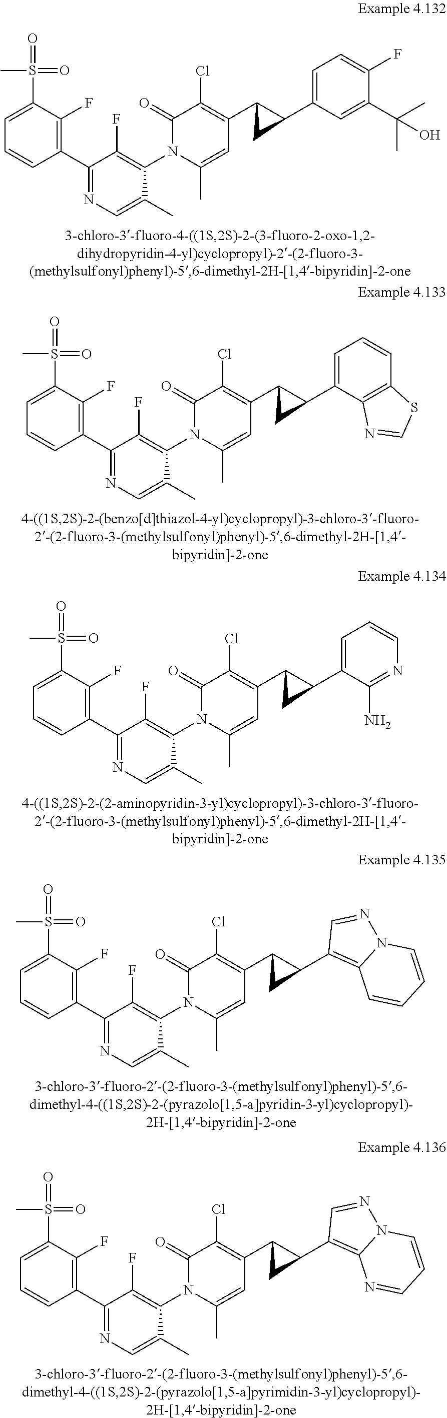

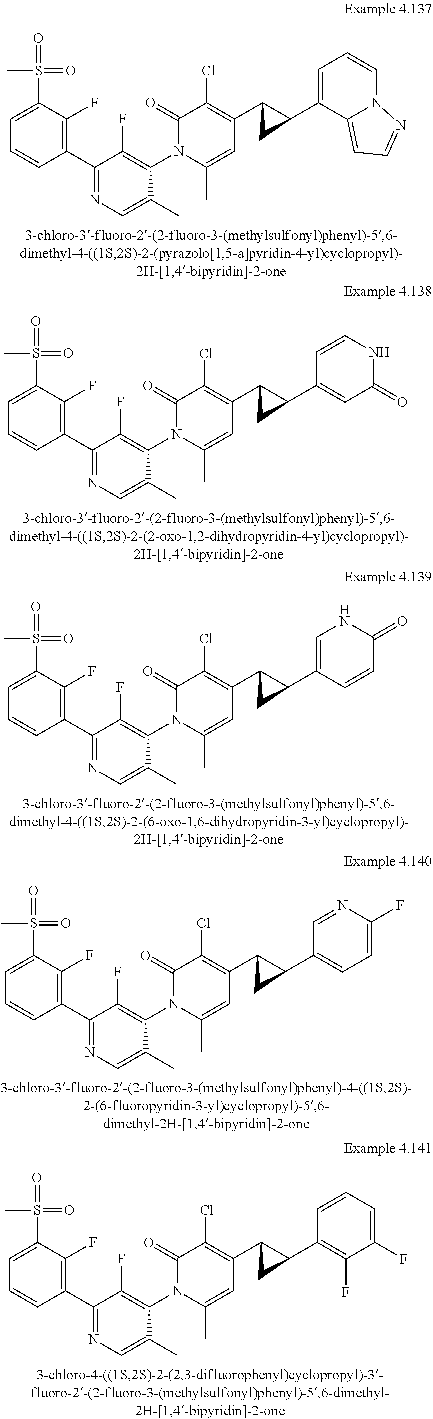

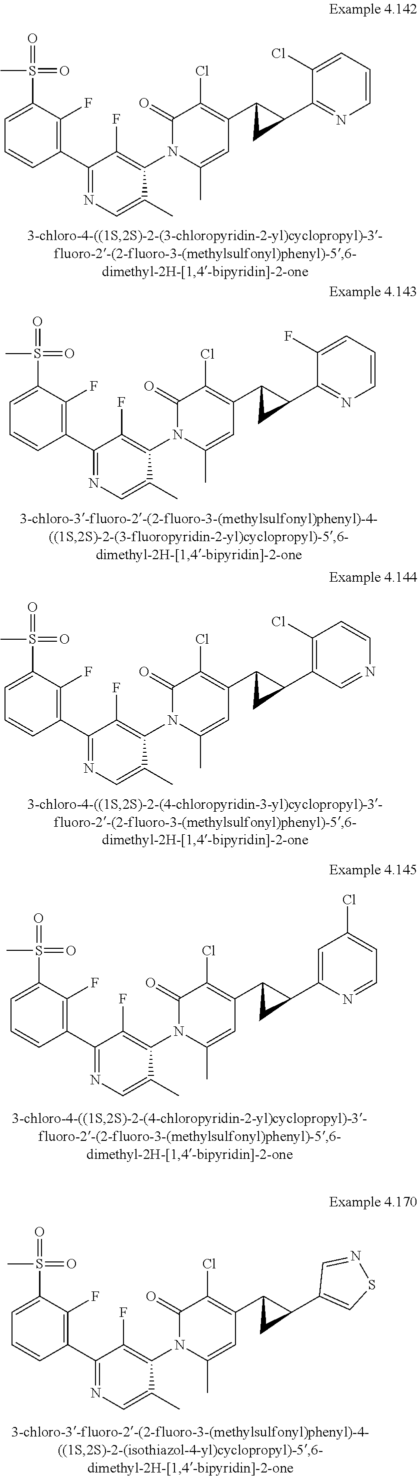

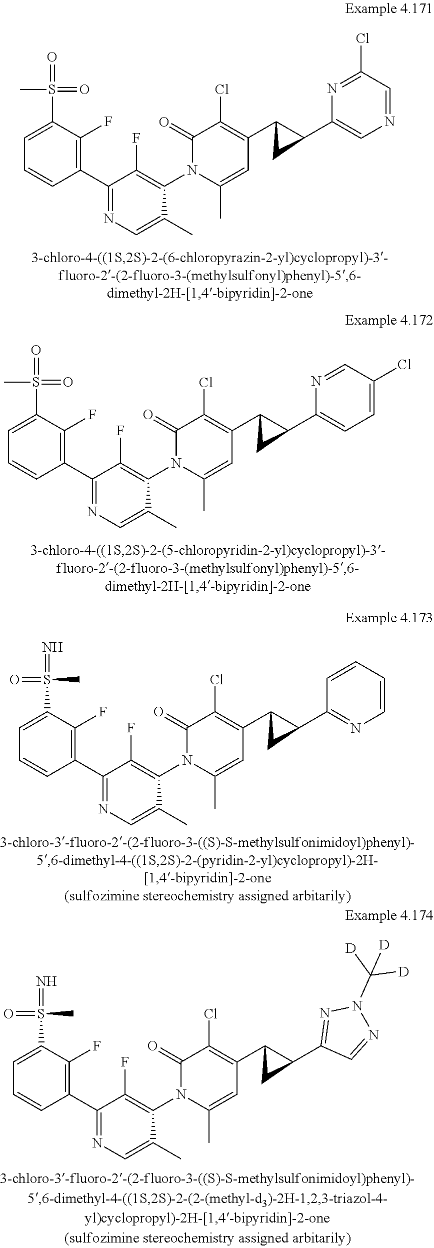

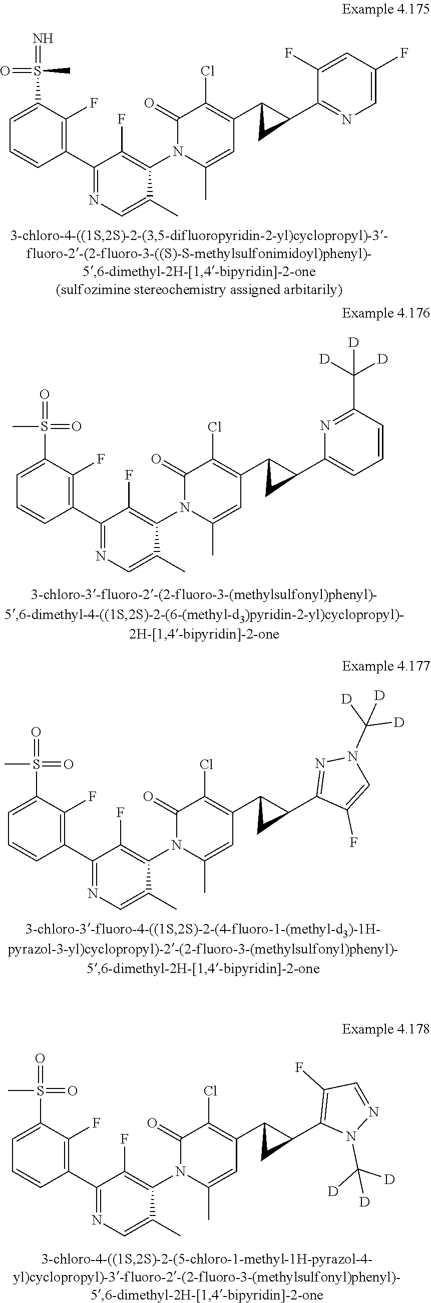

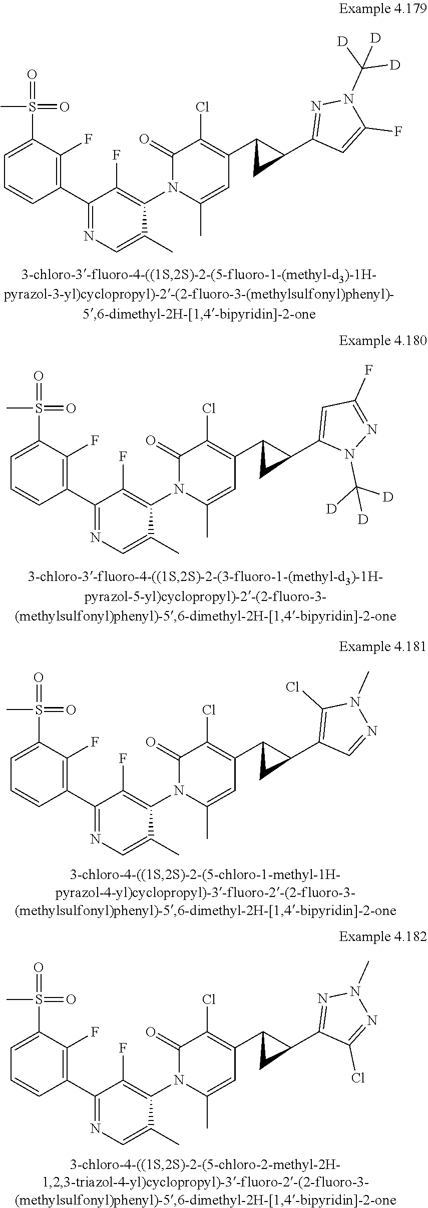

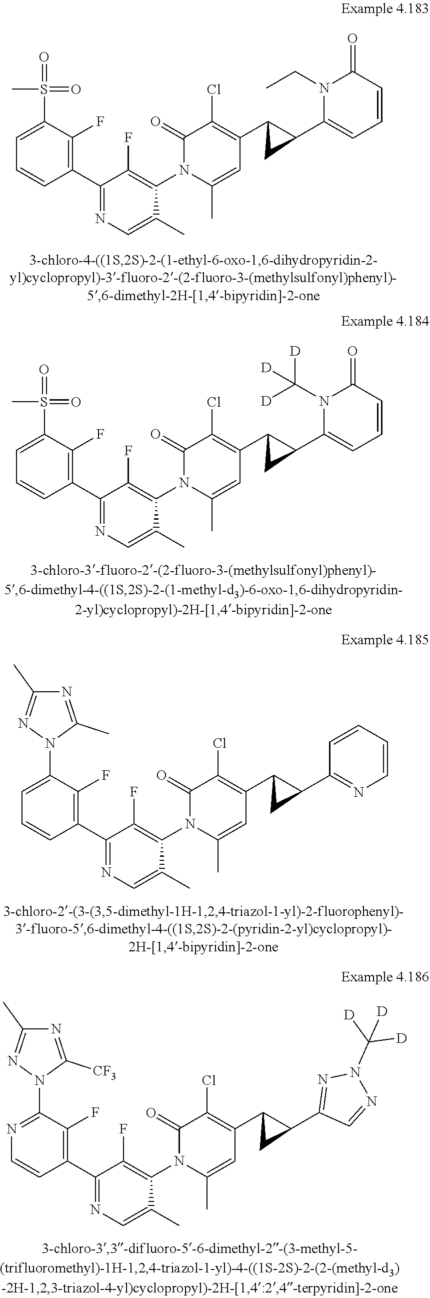

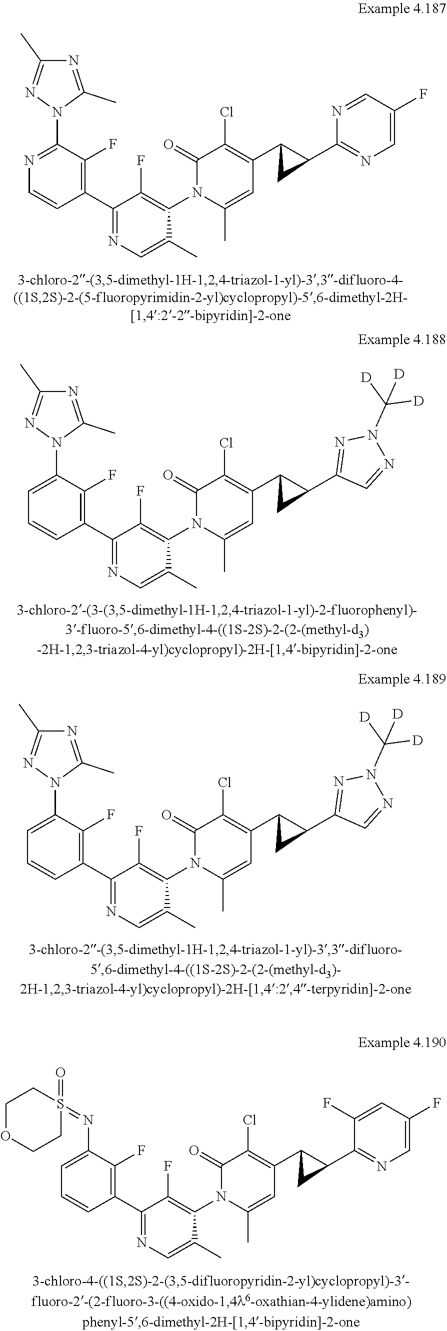

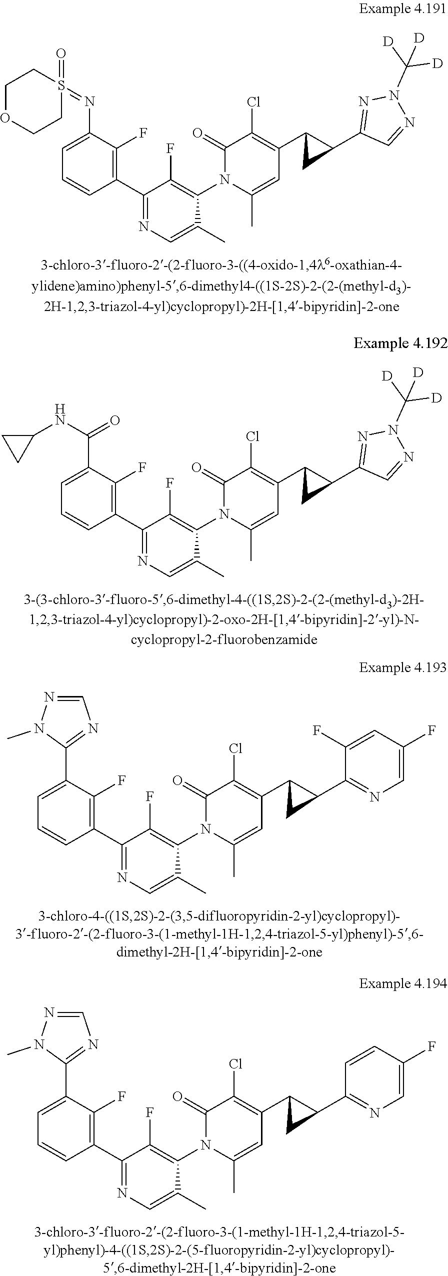

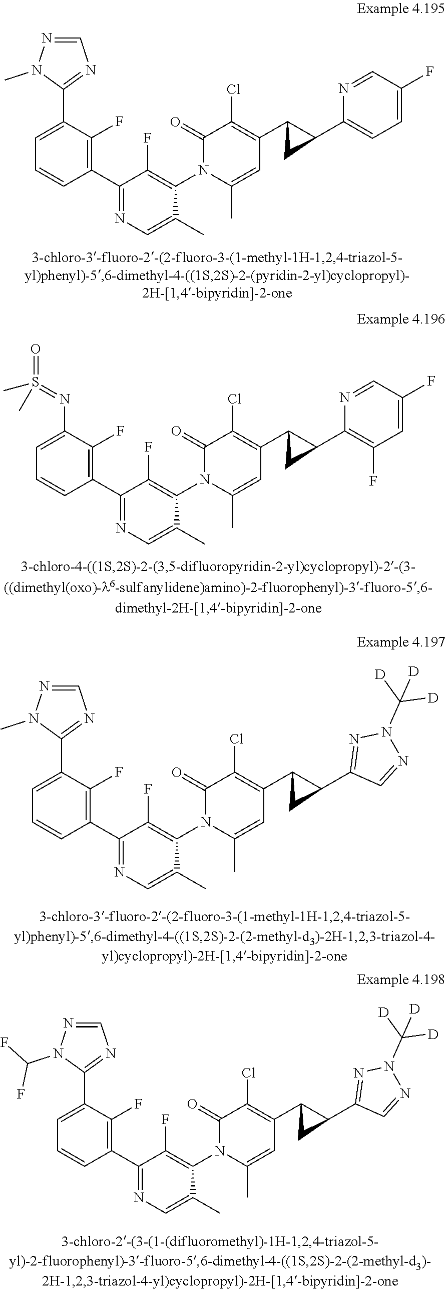

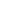

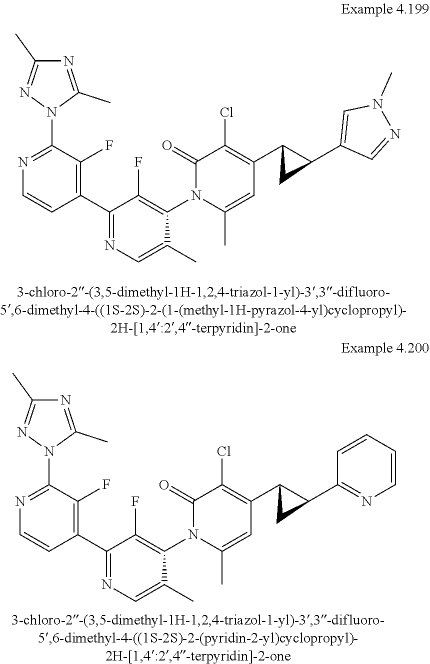

TABLE 3
Procedures analogous to Example 4.1 Synthesis

| Example | | Variations from Example 4.1 |
|---|---|---|
| Number | Aryl halide | Synthesis |
| 4.1 | 3-bromo-5,6-dihydro-4H-pyrrolo[1,2- | |
| | b]pyrazole | |
| 4.2 | 3-bromopyridine | D3.2 instead of D3.1 |
| 4.3 | 4-bromopyridine | D3.2 instead of D3.1 |
| 4.4 | 2-bromo-5-fluoropyridine | D3.2 instead of D3.1 |
| 4.5 | 3-bromo-5,6-dihydro-4H-pyrrolo[1,2- | D3.2 instead of D3.1 |
| | b]pyrazole | |
| 4.6 | 3-bromo-6,7-dihydro-5H-pyrrolo[1,2- | D3.2 instead of D3.1 |
| | a]imidazole | |
| 4.7 | 7-bromo-2,3-dihydropyrazolo[5,1-b]oxazole | D3.2 instead of D3.1 |
| 4.8 | 3-bromo-6,7-dihydro-5H-pyrazolo[5,1- | D3.2 instead of D3.1 |
| | b][1,3]oxazine | |
| 4.9 | 3-bromo-5-chloropyridine | |
| 4.10 | 7-bromoisoindolin-1-one | D3.2 instead of D3.1 |
| 4.11 | 4-bromoisoindolin-1-one | D3.2 instead of D3.1 |
| 4.12 | 5-bromo-3-chloropyridazine | |
| 4.13 | 2-bromo-6,7-dihydro-4H-pyrazolo[5,1- | |
| | c][1,4]oxazine | |
| 4.14 | 5-bromo-1-methyl-pyridin-2-one | |
| 4.15 | 2-bromo-6-chloropyrazine | |
| 4.16 | 3-bromo-5-chloro-2-methyl-pyridine | D3.2 instead of D3.1 |
| 4.17 | 3-bromo-5-fluoro-2-methyl-pyridine | D3.2 instead of D3.1 |
| 4.18 | 4-bromo-1,3-benzothiazole | D3.2 instead of D3.1 |
| 4.19 | 7-bromo-1,3-benzothiazole | D3.2 instead of D3.1 |
| 4.20 | 6-bromo-3-fluoro-2-methyl-pyridine | D3.2 instead of D3.1 |
| 4.21 | 4-bromo-2-methyl-oxazole | D3.2 instead of D3.1 |
| 4.22 | 3-bromo-4-fluoro-1-methyl-pyrazole | D3.2 instead of D3.1 |
| 4.23 | 3-bromo-5-chloropyridazine | |
| 4.24 | A13.1 | D3.2 instead of D3.1 |
| 4.25 | 3-bromo-5-fluoro-pyridine | D3.2 instead of D3.1 |
| 4.26 | 3-bromo-5-fluoro-pyridine-2-carbonitrile | D3.2 instead of D3.1 |
| 4.27 | 3-bromo-1-(difluoromethyl)-1H-pyrazole | D3.2 instead of D3.1 |
| 4.28 | 1-bromo-3-(difluoromethyl)benzene | D3.2 instead of D3.1 |
| 4.29 | 4-bromo-2-chloropyrimidine | |
| 4.30 | 4-bromo-6-chloropyrimidine | |
| 4.31 | 3-bromopyrazolo[1,5-a]pyridine | |
| 4.32 | 3-bromo-5,5-dimethyl-5,6-dihydro-4H- | |
| | pyrrolo[1,2-b]pyrazole | |
| 4.33 | 4-bromo-1-methyl-1H-pyrazole | |
| 4.34 | 3-bromoisothiazole | |
| 4.35 | C1.5 | |
| 4.36 | 4-bromoisothiazole | |
| 4.37 | 4-bromo-1-methyl-1H-1,2,3-triazole | |
| 4.38 | 5-bromo-2-methyl-2H-tetrazole | 24 hr reaction time |
| 4.39 | 5-bromoisothiazole | |
| 4.40 | 4-bromo-2-methyl-2H-1,2,3-triazole | 24 hr reaction time |
| 4.41 | 3-bromo-1,2,5-thiadiazole | 24 hr reaction time |
| 4.42 | 3-bromo-1-methyl-1,4,5,6- | 24 hr reaction time |
| | tetrahydrocyclopenta[c]pyrazole | |
| 4.43 | 7-bromobenzo[d]thiazole | |
| 4.44 | 4-bromoisoindolin-1-one | |
| 4.45 | 3-bromopyridin-2(1H)-one | |
| 4.46 | 4-bromo-2-methylthiazole | 24 hr reaction time |
| 4.47 | 4-bromoindolin-2-one | |
| 4.48 | 6-bromoindolin-2-one | |
| 4.49 | 4-bromo-2-chloropyridine | D3.2 instead of D3.1 |
| 4.50 | 5-bromo-2-chloropyridine | D3.2 instead of D3.1 |
| 4.51 | 4-bromo-6-chloropyridazin-3(2H)-one | |
| 4.52 | 4-bromo-2-(difluoromethyl)pyridine | D3.2 instead of D3.1 |
| 4.53 | 4-bromo-2-methoxypyridine | D3.2 instead of D3.1 |
| 4.54 | 5-bromo-2-fluoropyridine | D3.2 instead of D3.1 |
| 4.55 | 4-bromo-2-fluoropyridine | D3.2 instead of D3.1 |
| 4.56 | 3-bromo-1-((2- | D3.3 instead of D3.1; SEM product |
| | (trimethylsilyl)ethoxy)methyl)pyridin-2(1H)- | treated with 1:1 DCM/TFA for 2 |
| | one | hrs |
| 4.57 | 3-bromo-5-methyl-1-((2- | D3.3 instead of D3.1; SEM product |
| | (trimethylsilyl)ethoxy)methyl)pyridin-2(1H)- | treated with 1:1 DCM/TFA for 2 |
| | one | hrs |
| 4.58 | 3-bromo-5-fluoro-1-((2- | D3.3 instead of D3.1; SEM product |
| | (trimethylsilyl)ethoxy)methyl)pyridin-2(1H)- | treated with 1:1 DCM/TFA for 2 |
| | one | hrs |
| 4.59 | 3-bromo-5-(trifluoromethyl)-1-((2- | D3.3 instead of D3.1; SEM product |
| | (trimethylsilyl)ethoxy)methyl)pyridin-2(1H)- | treated with 1:1 DCM/TFA for 2 |
| | one | hrs |
| 4.60 | 3-bromo-2-((4-methoxybenzyl)oxy)pyridine | D3.4 instead of D3.1; PMB product |
| | | treated with 1:1 DCM/TFA for 2 |
| | | hrs |
| 4.61 | 2-bromo-6-((4-methoxybenzyl)oxy)pyridine | D3.4 instead of D3.1; PMB product |
| | | treated with 1:1 DCM/TFA for 2 |
| | | hrs |
| 4.62 | 3-bromo-5-(difluoromethyl)pyridine | D3.4 instead of D3.1 |
| 4.63 | 3-bromo-5-(trifluoromethyl)pyridine | D3.4 instead of D3.1 |
| 4.64 | 2-bromo-5-fluoro-4-methyl-pyrimidine | D3.4 instead of D3.1 |
| 4.65 | A13.7 | D3.4 instead of D3.1 |
| 4.66 | 2-bromo-6-methylpyrazine | D3.4 instead of D3.1 |
| 4.67 | 7-bromo-4-fluoropyrazolo[1,5-a]pyridine | D3.4 instead of D3.1 |
| 4.68 | A13.5 | D3.4 instead of D3.1 |
| 4.69 | 4-bromopyrimidine | D3.4 instead of D3.1 |
| 4.70 | 4-bromo-2,5-dimethyl-2H-1,2,3-triazole | D3.4 instead of D3.1 |
| 4.71 | 2-bromo-5-fluoropyrimidine | D3.4 instead of D3.1 |
| 4.72 | A13.3 | D3.4 instead of D3.1 |
| 4.73 | 5-bromopyrimidine | D3.4 instead of D3.1 |
| 4.74 | 3-bromo-1-methyl-1H-pyrazole-5-carbonitrile | D3.4 instead of D3.1 |
| 4.75 | 3-bromo-1,5-dimethyl-1H-pyrazole | D3.4 instead of D3.1 |
| 4.76 | 4-bromo-1,5-dimethyl-1H-pyrazole | D3.4 instead of D3.1 |
| 4.77 | 4-bromopyridazine | D3.4 instead of D3.1 |
| 4.78 | 2-bromo-6-methyl-pyridine | D3.4 instead of D3.1 |
| 4.79 | 4-bromo-5-fluoropyrimidine | D3.4 instead of D3.1 |
| 4.80 | 3-bromopyridazine | D3.4 instead of D3.1 |
| 4.81 | 4-bromo-1-cyclopropyl-pyrazole | D3.4 instead of D3.1 |
| 4.82 | 4-bromo-5-fluoro-1-methyl-pyrazole | D3.4 instead of D3.1 |
| 4.83 | 6-bromo-1-methylpyridin-2(1H)-one | D3.4 instead of D3.1 |
| 4.84 | A13.13 | D3.4 instead of D3.1 |
| 4.85 | A13.12 | D3.4 instead of D3.1 |
| 4.86 | A13.17 | D3.4 instead of D3.1 |
| 4.87 | A13.16 | D3.4 instead of D3.1 |
| 4.88 | A13.15 | D3.4 instead of D3.1 |
| 4.89 | A13.14 | D3.4 instead of D3.1 |
| 4.90 | 4-bromo-2-methylthiazole | D3.4 instead of D3.1 |
| 4.91 | 3-bromoisothiazole | D3.4 instead of D3.1 |
| 4.92 | 5-bromo-1-cyclopropyl-pyrazole | D3.4 instead of D3.1 |
| 4.93 | 5-bromo-3-fluoro-1-methyl-pyrazole | D3.4 instead of D3.1 |
| 4.94 | 2-(4-bromopyrazol-1-yl)acetonitrile | D3.4 instead of D3.1 |
| 4.95 | 4-bromo-1-methyl-pyrazole-3-carbonitrile | D3.4 instead of D3.1 |
| 4.96 | 3-bromo-2-methyl-indazole | D3.4 instead of D3.1 |
| 4.97 | 3-(4-bromopyrazol-1-yl)propanenitrile | D3.4 instead of D3.1 |
| 4.98 | 4-bromo-2-methyl-pyrazole-3-carbonitrile | D3.4 instead of D3.1 |
| 4.99 | 2-bromopyrazine | D3.4 instead of D3.1 |
| 4.100 | 4-bromoisoindoline-1,3-dione | D3.4 instead of D3.1 |
| 4.101 | A13.18 | D3.4 instead of D3.1. The product |
| | | was deprotected by stirring in TFA |
| | | for 2 hrs. |
| 4.102 | 3-iodo-1-methyl-1H-pyrazole-5-carboxamide | D3.4 instead of D3.1 |
| 4.103 | 2-bromopyrimidine | D3.4 instead of D3.1 |
| 4.104 | 2-bromo-4-methyl-pyrimidine | D3.4 instead of D3.1 |
| 4.105 | A13.25 | D3.4 instead of D3.1. The product |
| | | was deprotected by stirring in TFA |
| | | for 2 hrs. |
| 4.106 | A13.2 | D3.4 instead of D3.1 |
| 4.107 | tert-butyl (6-bromopyridin-2-yl)carbamate | D3.4 instead of D3.1. The product |
| | | was deprotected by stirring in |
| | | DCM/TFA for 1 hr. |
| 4.108 | 3-bromoimidazo[2,1-b]thiazole | D3.4 instead of D3.1 |
| 4.109 | 4-bromo-1-(tetrahydro-2H-pyran-2-y1)-1H- | D3.4 instead of D3.1; THP product |
| | pyrazole | was treated with 1:1 TFA/DCM for |
| | | 1 hr. |
| 4.110 | 2-bromo-3,5-difluoropyridine | D3.4 instead of D3.1 |
| 4.111 | A13.5 | D3.4 instead of D3.1 |
| 4.112 | A13.19 | D3.4 instead of D3.1. The product |
| | | was deprotected by stirring in TFA |
| | | for 2 hrs. |
| 4.113 | A13.20 | D3.4 instead of D3.1. The product |
| | | was deprotected by stirring in TFA |
| | | for 2 hrs. |
| 4.114 | A13.21 | D3.4 instead of D3.1. The product |
| | | was deprotected by stirring in TFA |
| | | for 2 hrs. |
| 4.115 | A13.22 | D3.4 instead of D3.1. The product |
| | | was deprotected by stirring in TFA |
| | | for 2 hrs. |
| 4.116 | A13.23 | D3.4 instead of D3.1. The product |
| | | was deprotected by stirring in TFA |
| | | for 2 hrs. |
| 4.117 | 6-bromopyrazolo[1,5-a]pyridine | D3.4 instead of D3.1 |
| 4.118 | 8-bromo-6-fluoro-[1,2,4]triazolo[1,5- | D3.4 instead of D3.1 |
| | a]pyridine | |
| 4.119 | 8-bromo-6-chloro-[1,2,4]triazolo[1,5- | D3.4 instead of D3.1 |
| | a]pyridine | |
| 4.120 | 4-bromo-6-fluoropyrazolo[1,5-a]pyridine | D3.4 instead of D3.1 |
| 4.121 | 4-bromopyrazolo[1,5-a]pyridine-3- | D3.4 instead of D3.1 |
| | carbonitrile | |
| 4.122 | 8-bromo-[1,2,4]triazolo[1,5-a]pyridine | D3.4 instead of D3.1 |
| 4.123 | 6-bromoimidazo[1,5-a]pyridine | D3.4 instead of D3.1 |
| 4.124 | 5-bromopyrazolo[1,5-a]pyridine | D3.4 instead of D3.1 |
| 4.125 | 3-bromopyrrolo[1,2-a]pyrimidine | D3.4 instead of D3.1 |
| 4.126 | 3-bromo-5,6-dihydro-4H-pyrrolo[1,2- | D3.4 instead of D3.1 |
| | b]pyrazole | |
| 4.127 | A13.25 | D3.4 instead of D3.1. The product |
| | | was deprotected by stirring in TFA |
| | | for 2 hrs. |
| 4.128 | A13.26 | D3.4 instead of D3.1. The product |
| | | was deprotected by stirring in TFA |
| | | for 2 hrs. |
| 4.129 | 3-bromoimidazo[1,2-b]pyridazine | D3.4 instead of D3.1 |
| 4.130 | A13.28 | D3.4 instead of D3.1. The product |
| | | was deprotected by stirring in TFA |
| | | for 2 hrs. |
| 4.131 | A13.27 | D3.4 instead of D3.1. The product |
| | | was deprotected by stirring in TFA |
| | | for 2 hrs. |
| 4.132 | 2-(5-bromo-2-fluorophenyl)propan-2-ol | D3.4 instead of D3.1 |
| 4.133 | 4-bromobenzo[d]thiazole | D3.4 instead of D3.1 |
| 4.134 | 3-bromopyridin-2-amine | D3.4 instead of D3.1 |
| 4.135 | 3-bromopyrazolo[1,5-a]pyridine | D3.4 instead of D3.1 |
| 4.136 | 3-bromopyrazolo[1,5-a]pyrimidine | D3.4 instead of D3.1 |
| 4.137 | 4-bromopyrazolo[1,5-a]pyridine | D3.4 instead of D3.1 |
| 4.138 | A13.30 | D3.4 instead of D3.1; PMB product |
| | | was treated with 1:1 TFA/DCM for |
| | | 2 hr. |
| 4.139 | A13.29 | D3.4 instead of D3.1; PMB product |
| | | was treated with 1:1 TFA/DCM for |
| | | 2 hr. |
| 4.140 | 5-bromo-2-fluoropyridine | D3.4 instead of D3.1 |
| 4.141 | 1-bromo-2,3-difluoro-benzene | D3.4 instead of D3.1 |
| 4.142 | 2-bromo-3-chloropyridine | D3.4 instead of D3.1 |
| 4.143 | 2-bromo-3-fluoropyridine | D3.4 instead of D3.1 |
| 4.144 | 3-bromo-4-chloropyridine | D3.4 instead of D3.1 |
| 4.145 | 2-bromo-4-chloropyridine | D3.4 instead of D3.1 |
| 4.146 | 4-bromo-3-fluoropyridine | D3.4 instead of D3.1 |
| 4.147 | 3-bromo-1-cyclopropylpyrazole | D3.4 instead of D3.1 |
| 4.148 | 4-bromo-3-fluoro-1-methyl-pyrazole | D3.4 instead of D3.1 |
| 4.149 | 3-bromo-1,4-dimethyl-pyrazole | D3.4 instead of D3.1 |
| 4.150 | 3-bromopyridine | D3.4 instead of D3.1 |
| 4.151 | 5-bromo-1-methyl-pyrazole | D3.4 instead of D3.1 |
| 4.152 | 3-bromo-1-methyl-pyrazole-4-carbonitrile | D3.4 instead of D3.1 |
| 4.153 | 2-bromo-6-chloropyridine | D3.4 instead of D3.1 |
| 4.154 | 2-bromopyridine | D3.4 instead of D3.1 |
| 4.155 | 2-bromo-5-fluoropyridine | D3.4 instead of D3.1 |
| 4.156 | 4-bromo-3-chloropyridine | D3.4 instead of D3.1 |
| 4.157 | 3-bromo-1-(difluoromethyl)-1H-pyrazole | D3.4 instead of D3.1 |
| 4.158 | 3-bromo-1-methyl-1H-pyrazole | D3.4 instead of D3.1 |
| 4.159 | 2-bromo-6-(difluoromethyl)pyridine | D3.4 instead of D3.1 |
| 4.160 | 2-bromo-6-(trifluoromethyl)pyrazine | D3.4 instead of D3.1 |
| 4.161 | 1-bromo-3,5-difluorobenzene | D3.4 instead of D3.1 |
| 4.162 | 1-bromo-2,4-difluorobenzene | D3.4 instead of D3.1 |
| 4.163 | 2-bromo-1,3-difluorobenzene | D3.4 instead of D3.1 |
| 4.164 | 1-bromo-2-fluorobenzene | D3.4 instead of D3.1 |
| 4.165 | 1-bromo-3-fluorobenzene | D3.4 instead of D3.1 |
| 4.166 | 4-bromo-1-methyl-1H-1,2,3-triazole | D3.4 instead of D3.1 |
| 4.167 | 4-bromo-2-methyl-oxazole | D3.4 instead of D3.1 |
| 4.168 | 7-bromo-2,3-dihydropyrazolo[5,1-b]oxazole | D3.4 instead of D3.1 |
| 4.169 | 3-bromo-4-fluoro-1-methyl-pyrazole | D3.4 instead of D3.1 |
| 4.170 | 4-bromoisothiazole | D3.4 instead of D3.1 |
| 4.171 | 2-bromo-6-chloro-pyrazine | D3.4 instead of D3.1 |
| 4.172 | 2-bromo-5-chloropyridine | D3.4 instead of D3.1 |
| 4.173 | 2-bromopyridine | D3.5a instead of D3.1; Boc product |
| | | was treated with 1:2 TFA/DCM for |
| | | 10 min. |
| 4.174 | A13.2 | D3.5a instead of D3.1; Boc product |
| | | was treated with 1:2 TFA/DCM for |
| | | 10 min. |
| 4.175 | 2-bromo-3,5-difluoro-pyridine | D3.5a instead of D3.1; Boc product |
| | | was treated with 1:2 TFA/DCM for |
| | | 10 min. |
| 4.176 | A13.4 | D3.4 instead of D3.1 |
| 4.177 | A13.11 | D3.4 instead of D3.1 |
| 4.178 | A13.10 | D3.4 instead of D3.1 |
| 4.179 | A13.9 | D3.4 instead of D3.1 |
| 4.180 | A13.8 | D3.4 instead of D3.1 |
| 4.181 | 4-bromo-5-chloro-1-methyl-1H-pyrazole | D3.4 instead of D3.1 |
| 4.182 | 4-bromo-5-chloro-2-methyl-2H-1,2,3-triazole | D3.4 instead of D3.1 |
| 4.183 | 6-bromo-1-ethylpyridin-2(1H)-one | D3.4 instead of D3.1 |
| 4.184 | 6-bromo-1-(methyl-d3)pyridin-2(1H)-one | D3.4 instead of D3.1 |
| 4.185 | 2-bromopyridine | D3.11 instead of D3.1 |
| 4.186 | A13.2 | D3.10 instead of D3.1 |
| 4.187 | 2-bromo-5-fluoropyrimidine | D3.8 instead of D3.1 |
| 4.188 | A13.2 | D3.11 instead of D3.1 |
| 4.189 | A13.2 | D3.8 instead of D3.1 |
| 4.190 | 2-bromo-3,5-difluoropyridine | D3.13 instead of D3.1 |
| 4.191 | A13.2 | D3.13 instead of D3.1 |
| 4.192 | A13.2 | D3.7 instead of D3.1 |
| 4.193 | 2-bromo-3,5-difluoropyridine | D3.12 instead of D3.1 |
| 4.194 | 2-bromo-5-fluoropyridine | D3.12 instead of D3.1 |
| 4.195 | 2-bromopyridine | D3.12 instead of D3.1 |
| 4.196 | 2-bromo-3,5-difluoropyridine | D3.9 instead of D3.1 |
| 4.197 | A13.2 | D3.12 instead of D3.1 |
| 4.198 | A13.2 | D3.6 instead of D3.1 |
| 4.199 | 4-bromo-1-methyl-1H-pyrazole | D3.8 instead of D3.1 |
| 4.200 | 2-bromopyridine | D3.8 instead of D3.1 |

### Example 5.1: 3-((S)-3-chloro-4-((1S,2S)-2-(4-fluorophenyl)cyclopropyl)-5′,6-dimethyl-2-oxo-2H-[1,4′-bipyridin]-2′-yl)-2-fluorobenzamide

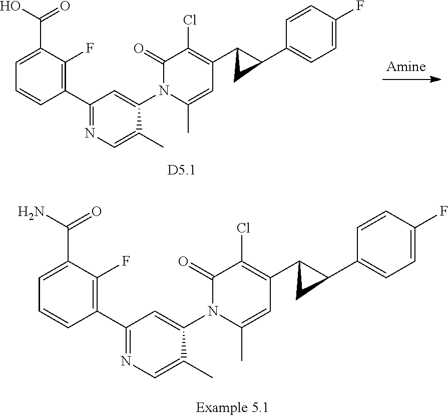

3-((S)-3-chloro-4-((1S,2S)-2-(4-fluorophenyl)cyclopropyl)-5′,6-dimethyl-2-oxo-2H-[1,4′-bipyridin]-2′-yl)-2-fluorobenzoic acid (D5.1) (95 mg, 0.19 mmol) was charged to a vial, followed by solid NH₄Cl (Amine) (30 mg, 0.56 mmol), and HATU (66 mg, 0.280 mmol). The solids were dissolved in DMF (5 mL) and N,N-diisopropylamine (0.16 mL, 0.935 mmol) was added and the reaction was stirred overnight at room temperature. The reaction was concentrated and subjected to reverse phase HPLC (Silicycle SiliaChrom SB C18, 5 μm, 150 Å, 30×250 mm; 0.1% TFA in MeCN-0.1% TFA in water) to give 3-[4-[3-chloro-4-[(1S,2S)-2-(4-fluorophenyl)cyclopropyl]-6-methyl-2-oxo-1-pyridyl]-5-methyl-2-pyridyl]-2-fluoro-benzamide (Example 5.1).

The following compounds in Table 4 were prepared analogously to Example 5.1 modified as

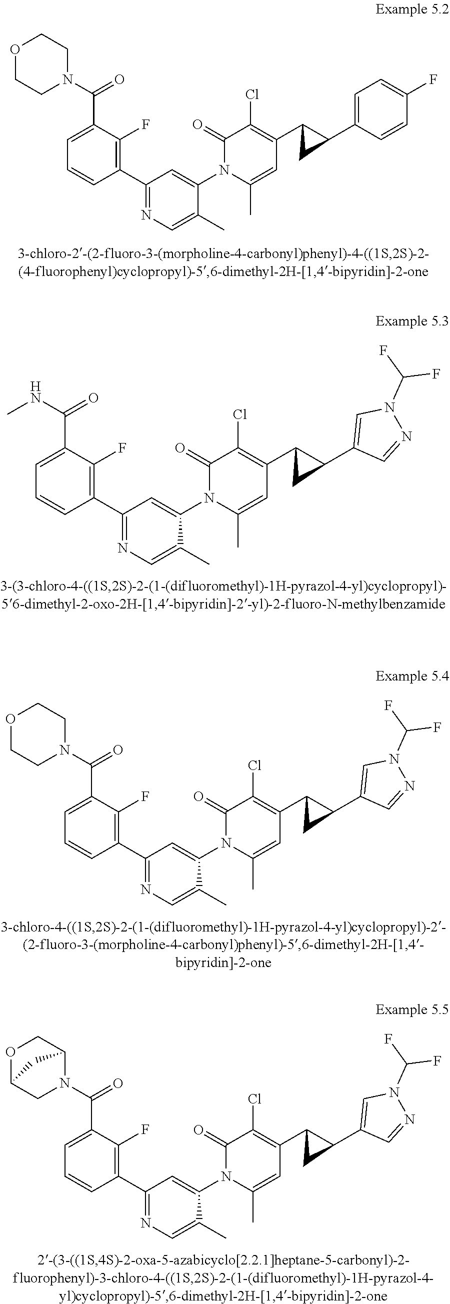

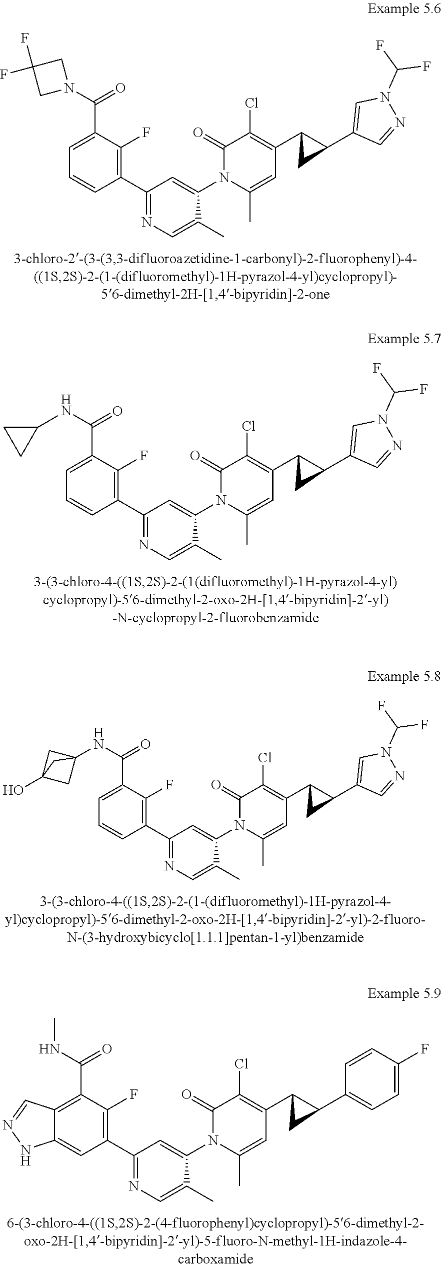

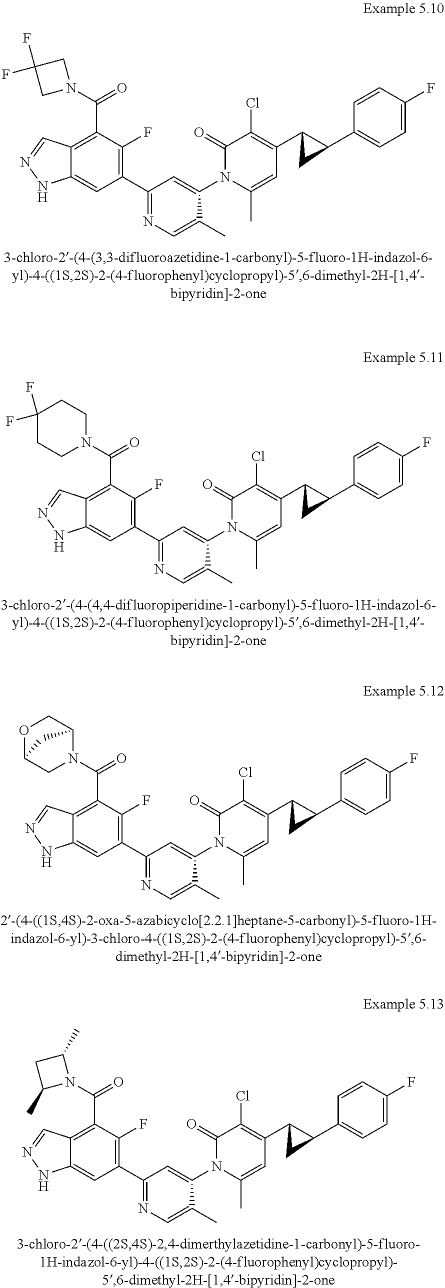

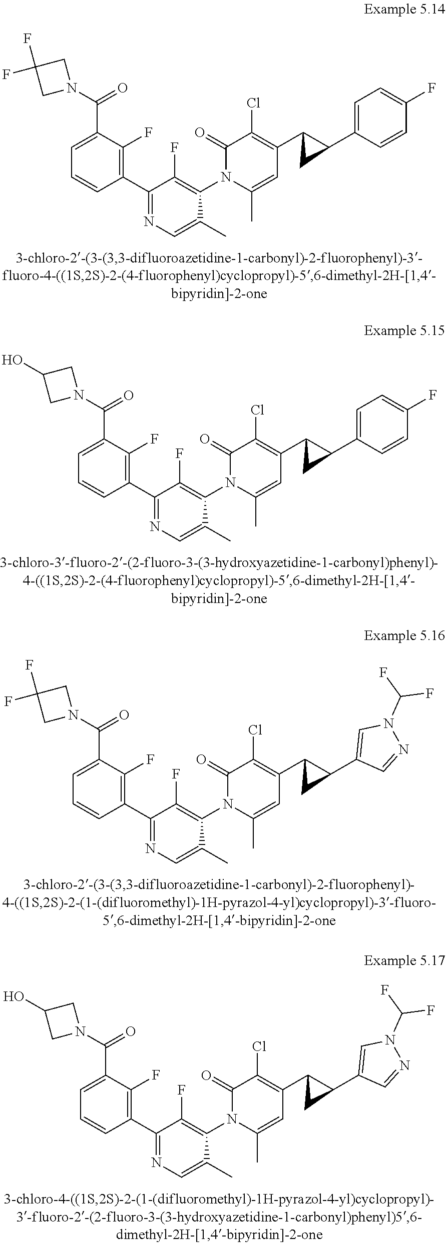

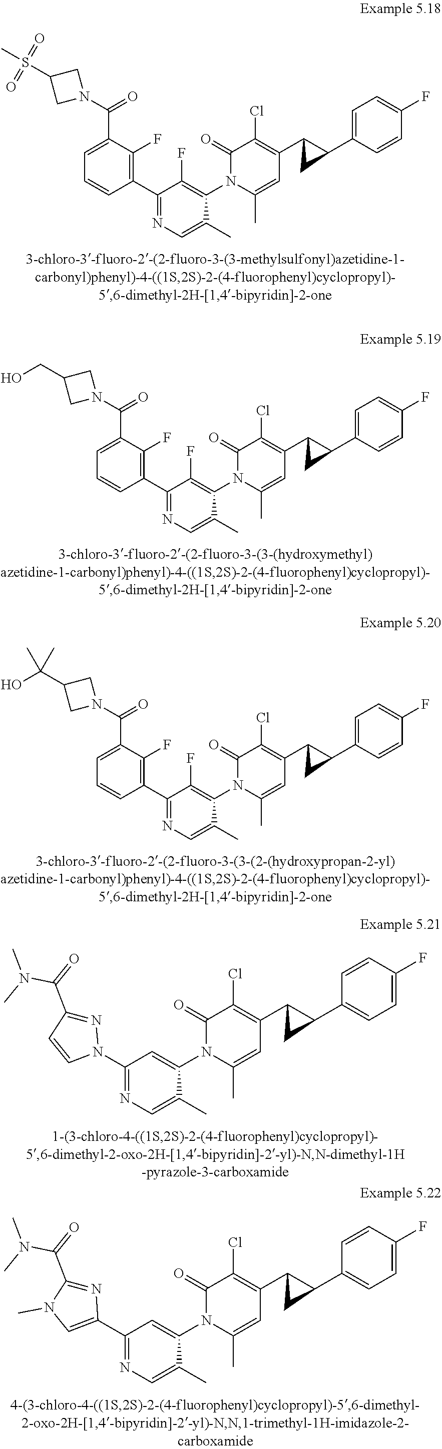

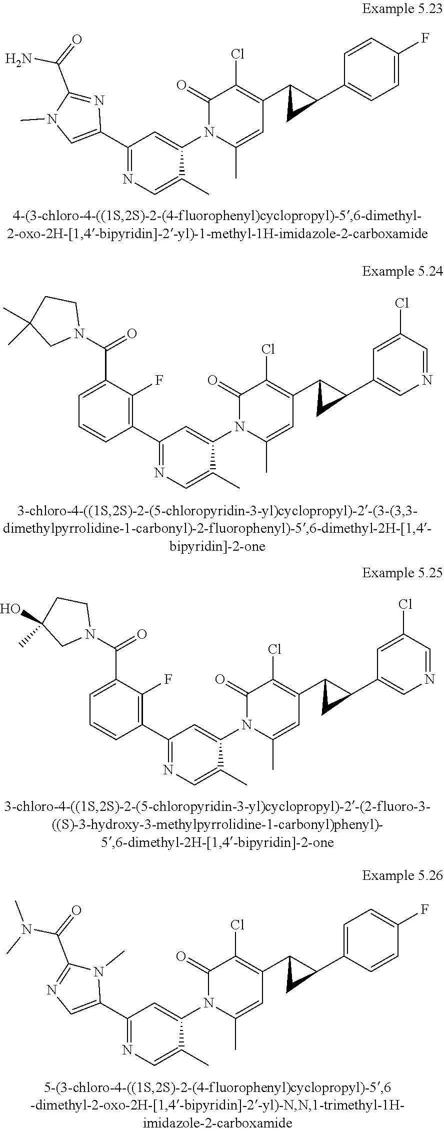

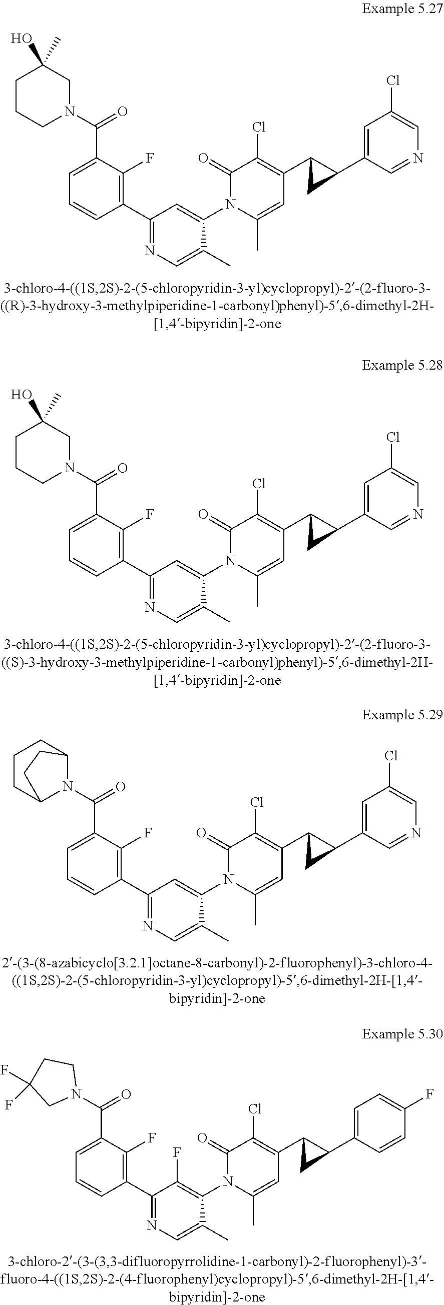

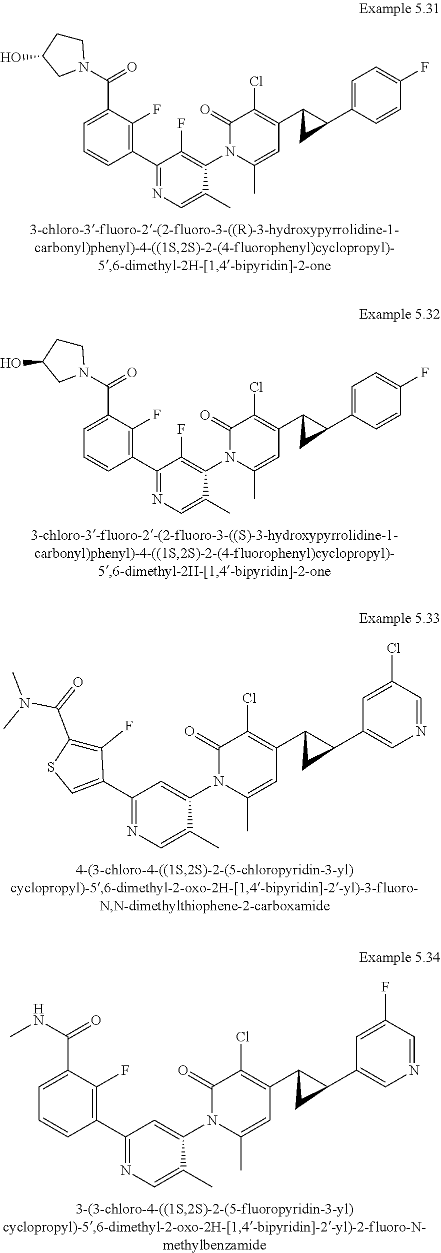

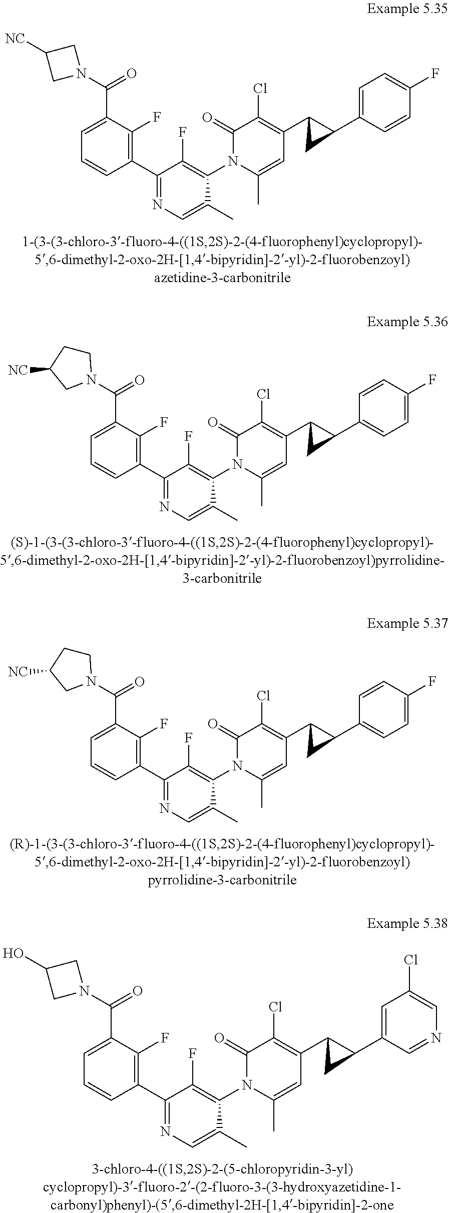

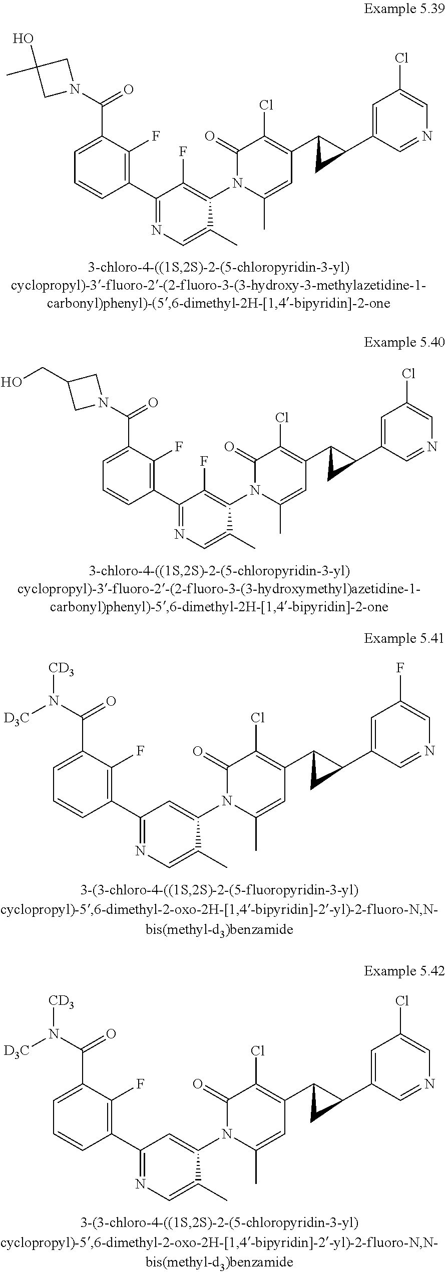

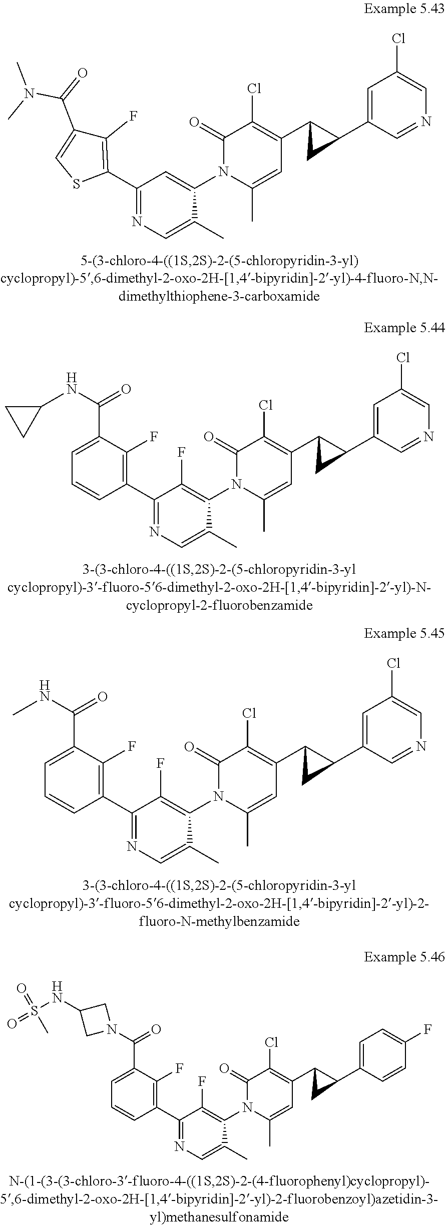

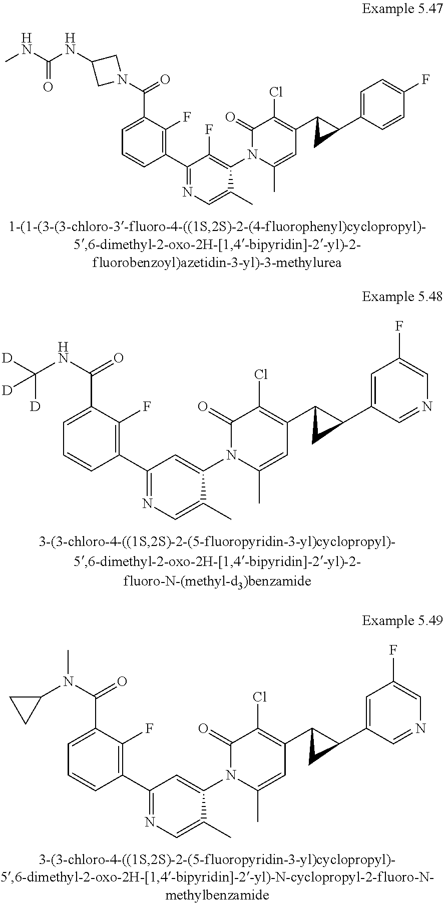

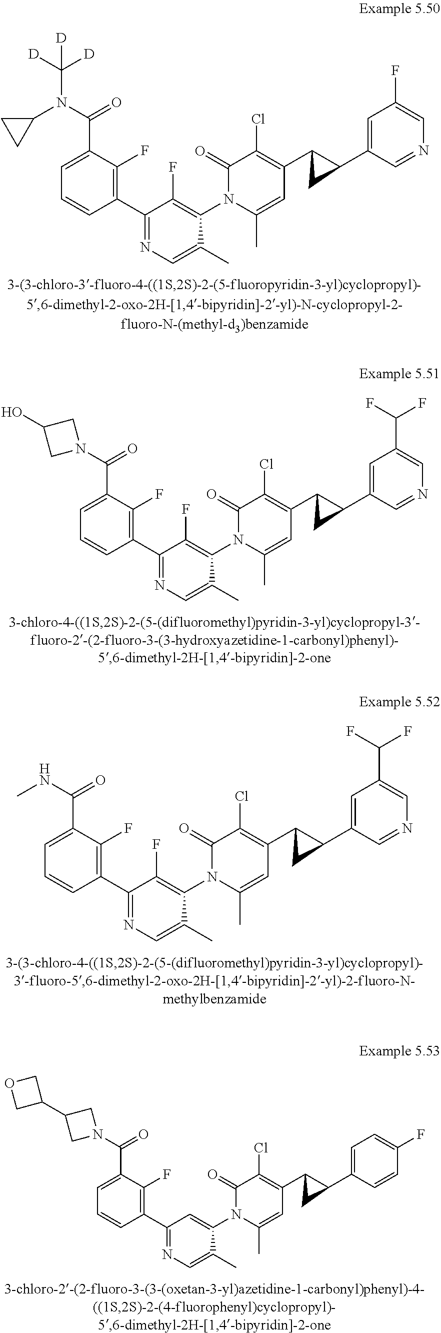

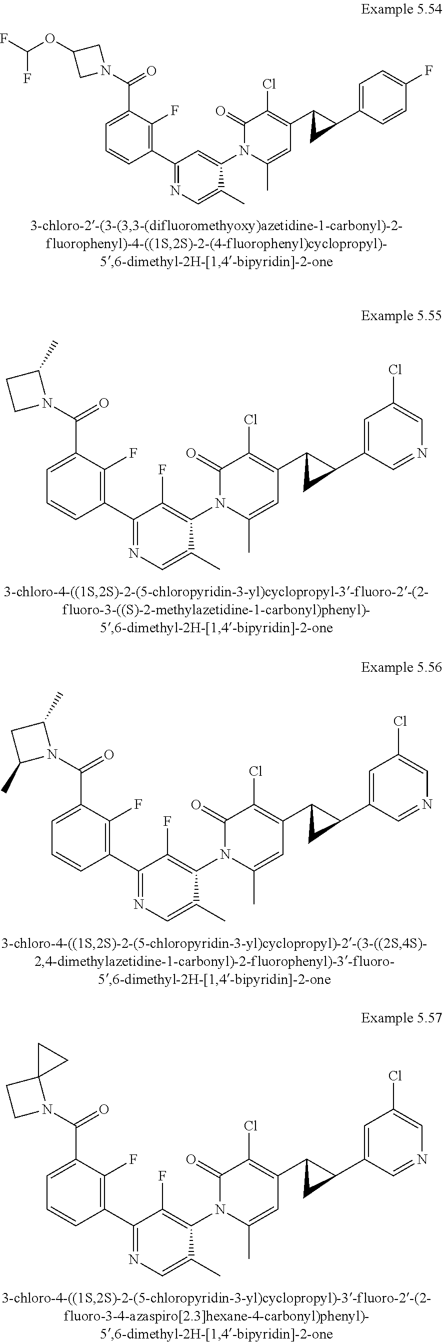

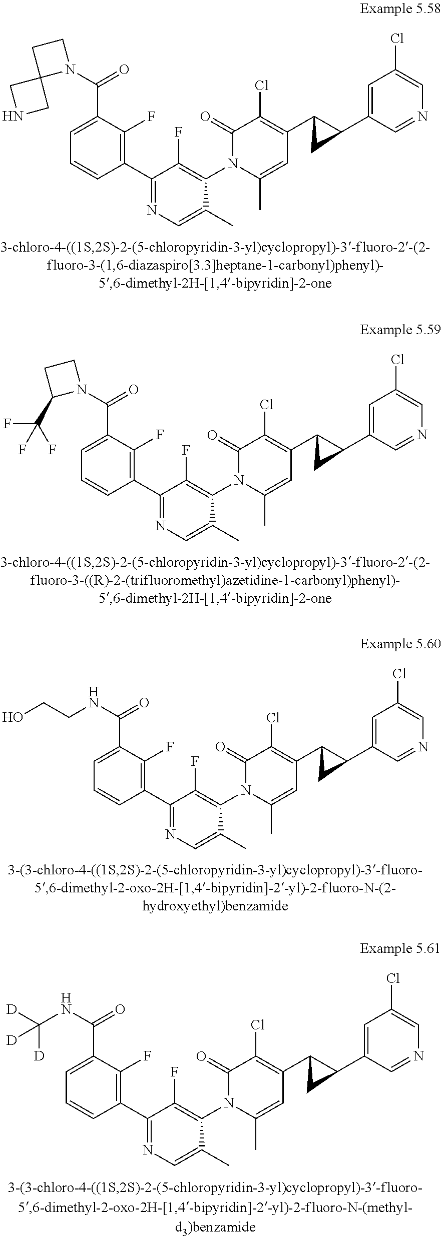

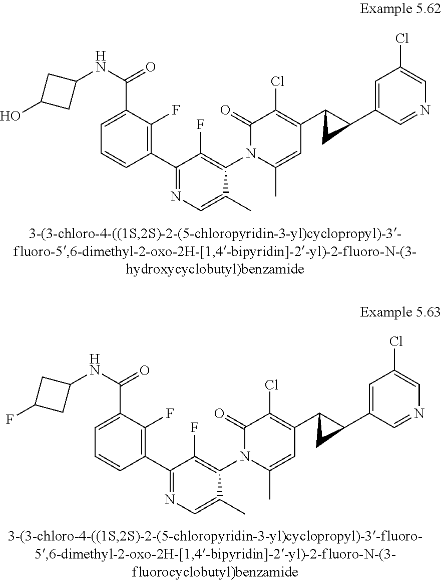

TABLE 4
Procedures analogous to Example 5.1 Synthesis

| Example | Starting | | |
|---|---|---|---|
| Number | Material | Amine | Variation from Example 5.1 |
| 5.2 | (rac)-D5.1 | morpholine | |
| 5.3 | D5.3 | methylamine hydrochloride | |
| 5.4 | D5.3 | morpholine | |
| 5.5 | D5.3 | (1S,4S)-2-oxa-5- | |
| | | azabicyclo[2.2.1]heptane | |
| | | hydrochloride | |
| 5.6 | D5.3 | 3,3-difluoroazetidine hydrochloride | |
| 5.7 | D5.3 | cyclopropylamine | |
| 5.8 | D5.3 | 3-aminobicyclo[1.1.1]pentan-1-ol | |
| | | hydrochloride | |
| 5.9 | D5.13 | methylamine | Treated with HCl in EtOH (10 |
| | | | equiv) at 60° C. for 16 hr |
| | | | following amide coupling |
| 5.10 | D5.13 | 3,3-difluoroazetidine | Treated with HCl in EtOH (10 |
| | | | equiv) at 60° C. for 16 hr |
| | | | following amide coupling |
| 5.11 | D5.13 | 4,4-difluoropiperidine | Treated with HCl in EtOH (10 |
| | | | equiv) at 60° C. for 16 hr |
| | | | following amide coupling |
| 5.12 | D5.13 | (1S,4S)-2-oxa-5- | Treated with HCl in EtOH (10 |
| | | azabicyclo[2.2.1]heptane | equiv) at 60° C. for 16 hr |
| | | | following amide coupling |
| 5.13 | D5.13 | (2S,4S)-2,4-dimethylazetidine | Treated with HCl in EtOH (10 |
| | | | equiv) at 60° C. for 16 hr |
| | | | following amide coupling |
| 5.14 | D5.2 | 3,3-difluoroazetidine hydrochloride | |
| 5.15 | D5.2 | azetidin-3-ol hydrochloride | |
| 5.16 | D5.4 | 3,3-difluoroazetidine hydrochloride | |
| 5.17 | D5.4 | azetidin-3-ol hydrochloride | |
| 5.18 | D5.2 | 3-(methylsulfonyl)azetidine | |
| | | hydrochloride | |
| 5.19 | D5.2 | azetidin-3-ylmethanol hydrochloride | |
| 5.20 | D5.2 | 2-(azetidin-3-yl)propan-2-ol | |
| | | hydrochloride | |
| 5.21 | D5.19 | dimethylamine | |
| 5.22 | D5.11 | dimethylamine | |
| 5.23 | D5.11 | ammonia, 2M in iPrOH | |
| 5.24 | D5.5 | 3,3-dimethylpyrrolidine | |
| 5.25 | D5.5 | (3S)-3-methylpyrrolidin-3-ol | |
| 5.26 | D5.12 | dimethylamine hydrochloride | |
| 5.27 | D5.5 | (3R)-3-methylpiperidin-3-ol | |
| 5.28 | D5.5 | (3S)-3-methylpiperidin-3-ol | |
| 5.29 | D5.5 | 8-azabicyclo[3.2.1]octane | |
| 5.30 | D5.2 | 3,3-difluoropyrrolidine hydrochloride | |
| 5.31 | D5.2 | (R)-pyrrolidin-3-ol hydrochloride | |
| 5.32 | D5.2 | (S)-pyrrolidin-3-ol hydrochloride | |
| 5.33 | D5.9 | dimethylamine | |
| 5.34 | D5.7 | methylamine | |
| 5.35 | D5.2 | azetidin-3-carbonitrile hydrochloride | |
| 5.36 | D5.2 | (S)-pyrrolidine-3-carbonitrile | |
| | | hydrochloride | |
| 5.37 | D5.2 | (R)-pyrrolidine-3-carbonitrile | |
| | | hydrochloride | |
| 5.38 | D5.6 | azetidin-3-ol hydrochloride | |
| 5.39 | D5.6 | 3-methylazetidin-3-ol hydrochloride | |
| 5.40 | D5.6 | azetidin-3-ylmethanol hydrochloride | |
| 5.41 | D5.7 | bis(methyl-d₃)amine hydrochloride | |
| 5.42 | D5.5 | bis(methyl-d₃)amine hydrochloride | |
| 5.43 | D5.10 | dimethylamine | |
| 5.44 | D5.6 | cyclopropylamine | |
| 5.45 | D5.6 | methylamine hydrochloride | |
| 5.46 | D5.2 | N-(azetidin-3-yl)methanesulfonamide | |
| 5.47 | D5.2 | 1-(azetidin-3-y1)-3-methylurea | |
| 5.48 | D5.7 | methan-d3-amine | |
| 5.49 | D5.7 | N-methylcyclopropanamine | |
| 5.50 | D5.8 | N-(methyl-d3)cyclopropanamine | |
| 5.51 | D5.14 | azetidin-3-ol | |
| 5.52 | D5.14 | methylamine | |
| 5.53 | D5.2 | 3-(oxetan-3-yl)azetidine | |
| 5.54 | D5.2 | 3-(difluoroaethoxy)azetidine | |
| 5.55 | D5.6 | (S)-2-methylazetidine | |
| 5.56 | D5.6 | (2S,4S)-2,4-dimethylazetidine | |
| 5.57 | D5.6 | 4-azaspiro[2.3]hexane | |
| 5.58 | D5.6 | tert-butyl 1,6-diazaspiro[3.3]heptane- | Treated with TFA/DCM for 15 |
| | | 6-carboxylate | min following amide coupling |
| 5.59 | D5.6 | (R)-2-(trifluoromethyl)azetidine | |
| 5.60 | D5.6 | ethanolamine | |
| 5.61 | D5.6 | methan-d3-amine | |
| 5.62 | D5.6 | 3-aminocyclobutan-1-ol | |
| 5.63 | D5.6 | 3-fluorocyclobutan-1-amine | |

### Example 6.1: 6-(3-chloro-4-((1S,2S)-2-(4-fluorophenyl)cyclopropyl)-5′,6-dimethyl-2-oxo-2H-[1,4′-bipyridin]-2′-yl)-7-fluorobenzo[d]oxazol-2(3H)-one

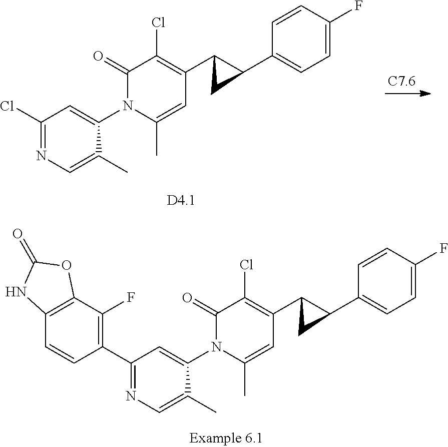

A vial was charged with 2′,3-dichloro-4-((1S,2S)-2-(4-fluorophenyl)cyclopropyl)-5′,6-dimethyl-2H-[1,4′-bipyridin]-2-one (D4.1) (25 mg, 0.062 mmol), 7-fluoro-6-(4,4,5,5-tetramethyl-1,3,2-dioxaborolan-2-yl)benzo[d]oxazol-2(3H)-one (C7.6) (26 mg, 0.093 mmol), and Pd(PPh₃)₄ (7.2 mg, 0.0062 mmol), followed by 1,2-DME (2 mL), and Na₂CO₃ (2M aq., 0.124 mL). The mixture was heated to 85° C. for 1.5 hrs. The mixture was cooled to ambient temperature, concentrated and subjected to flash column chromatography (SiO₂, hexanes-EtOAc), followed by reverse phase HPLC (Silicycle SiliaChrom SB C18, 5 μm, 150 Å, 30×250 mm; 0.1% TFA in MeCN-0.1% TFA in water) to give 6-((S)-3-chloro-4-((1S,2S)-2-(4-fluorophenyl)cyclopropyl)-5′,6-dimethyl-2-oxo-2H-[1,4′-bipyridin]-2′-yl)-7-fluorobenzo[d]oxazol-2(3H)-one (Example 6.1).

The following compounds in Table 5 were prepared analogously to Example 6.1 modified as described:

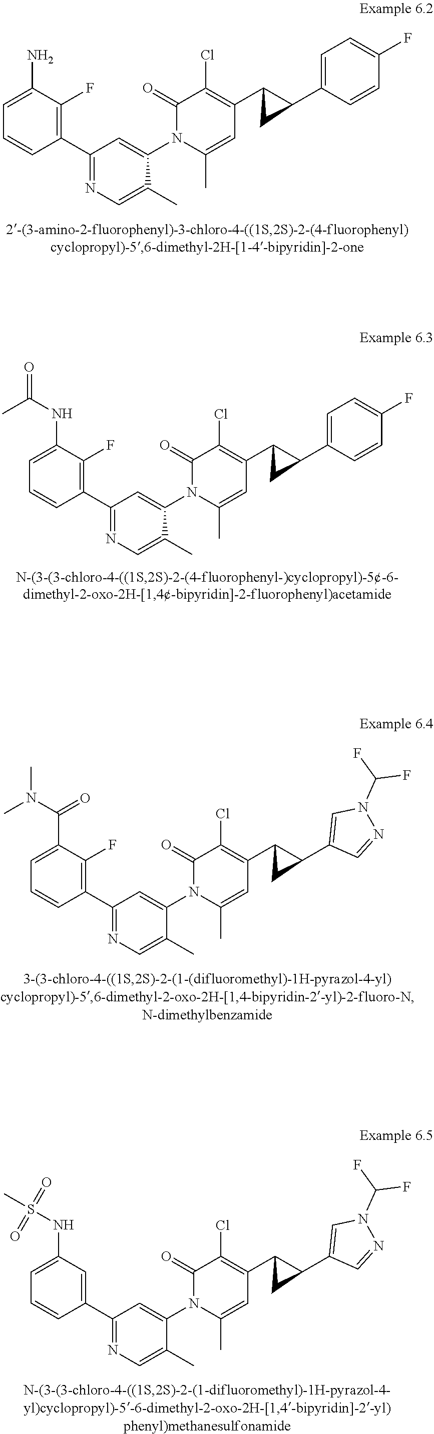

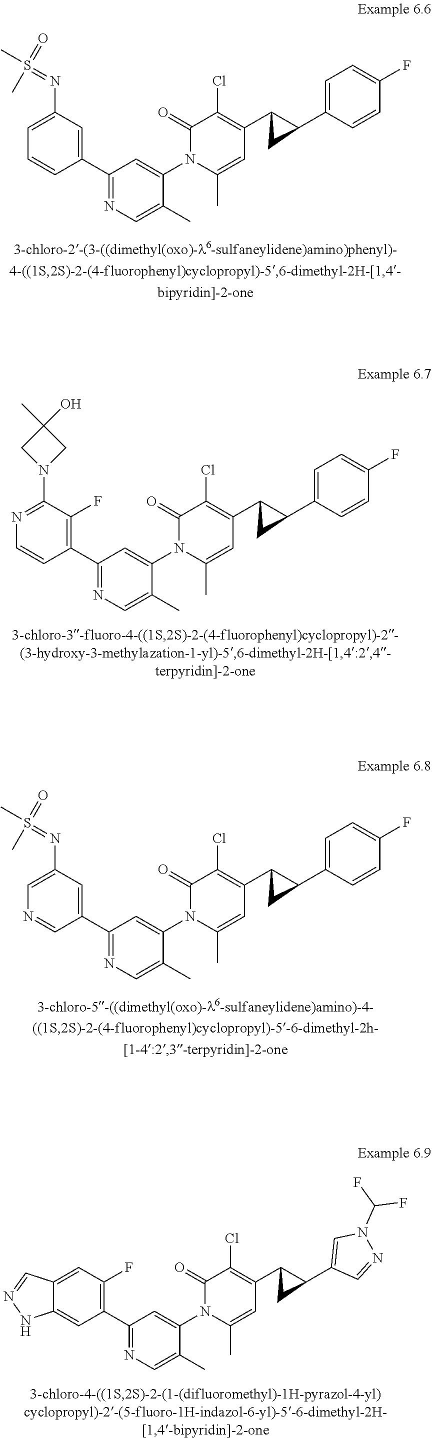 [SMILES: Cc1cnc(-c2cccc(N=S(C)(C)=O)c2)cc1-n1c(C)cc([C@H]2C[C@@H]2c2ccc(F)cc2)c(Cl)c1=O ; Cc1cnc(-c2ccnc(N3CC(C)(O)C3)c2F)cc1-n1c(C)cc([C@H]2C[C@@H]2c2ccc(F)cc2)c(Cl)c1=O ; Cc1cnc(-c2cncc(N=S(C)(C)=O)c2)cc1-n1c(C)cc([C@H]2C[C@@H]2c2ccc(F)cc2)c(Cl)c1=O ; Cc1cnc(-c2cc3[nH]ncc3cc2F)cc1-n1c(C)cc([C@H]2C[C@@H]2c2cnn(C(F)F)c2)c(Cl)c1=O]

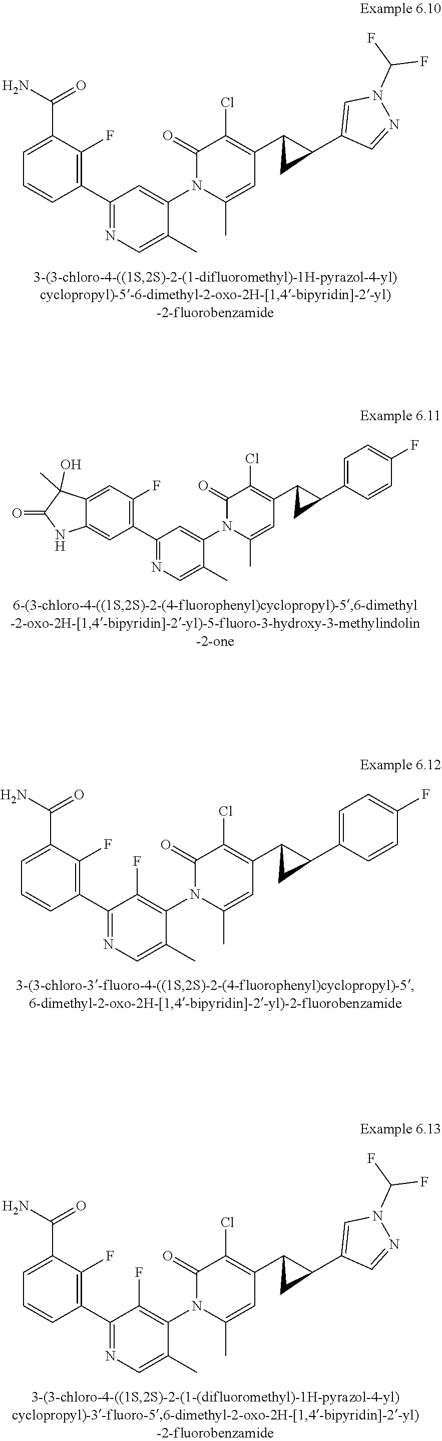 [SMILES: Cc1cnc(-c2cccc(C(N)=O)c2F)cc1-n1c(C)cc([C@H]2C[C@@H]2c2cnn(C(F)F)c2)c(Cl)c1=O ; Cc1cnc(-c2cc3c(cc2F)C(C)(O)C(=O)N3)cc1-n1c(C)cc([C@H]2C[C@@H]2c2ccc(F)cc2)c(Cl)c1=O ; Cc1cnc(-c2cccc(C(N)=O)c2F)c(F)c1-n1c(C)cc([C@H]2C[C@@H]2c2ccc(F)cc2)c(Cl)c1=O ; Cc1cnc(-c2cccc(C(N)=O)c2F)c(F)c1-n1c(C)cc([C@H]2C[C@@H]2c2cnn(C(F)F)c2)c(Cl)c1=O]

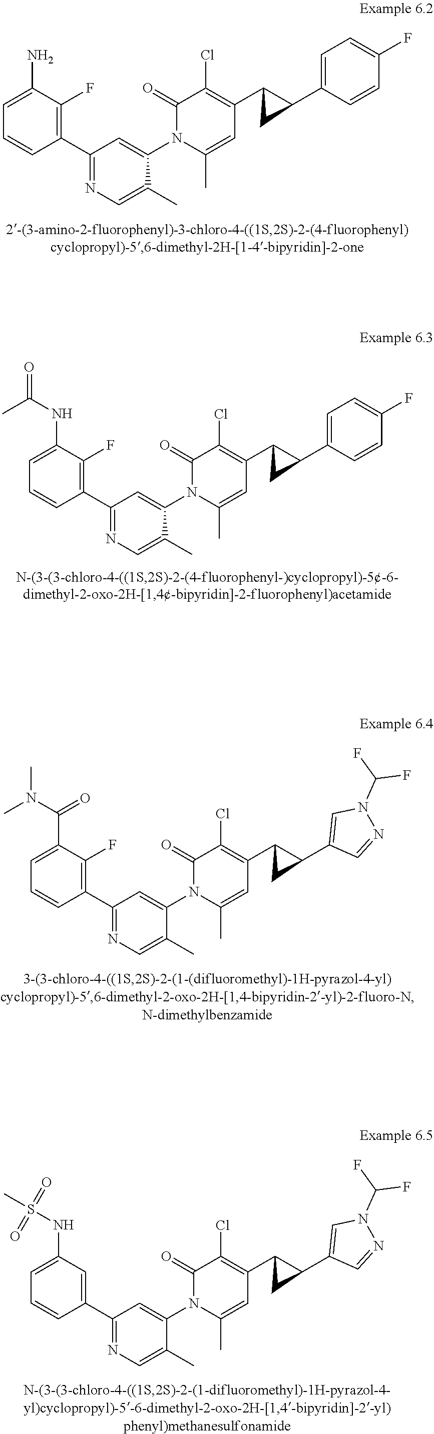

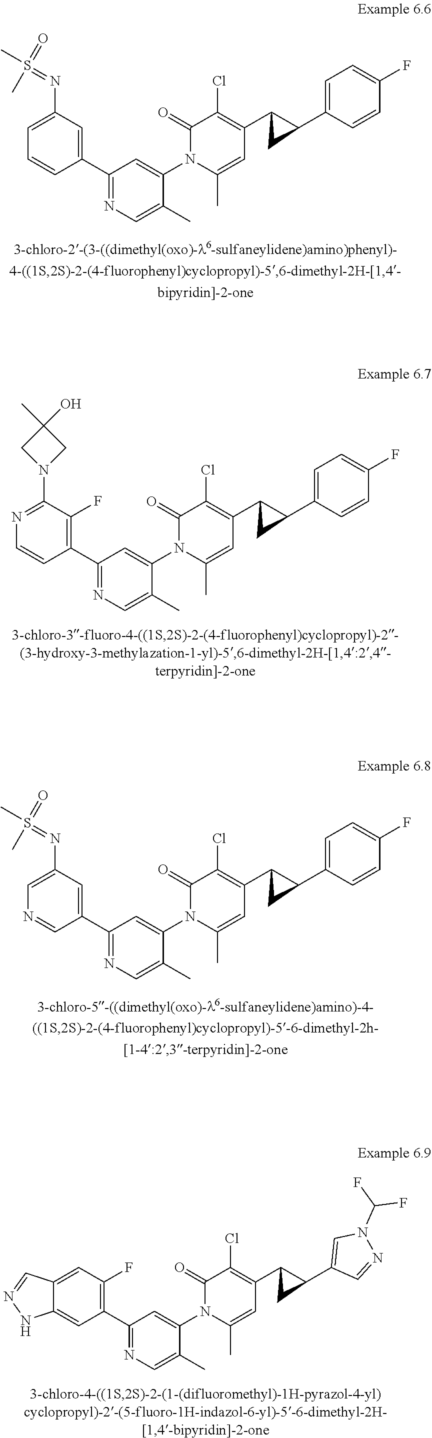 [SMILES: Cc1cnc(-c2cccc(N=S(C)(C)=O)c2)cc1-n1c(C)cc([C@H]2C[C@@H]2c2ccc(F)cc2)c(Cl)c1=O ; Cc1cnc(-c2ccnc(N3CC(C)(O)C3)c2F)cc1-n1c(C)cc([C@H]2C[C@@H]2c2ccc(F)cc2)c(Cl)c1=O ; Cc1cnc(-c2cncc(N=S(C)(C)=O)c2)cc1-n1c(C)cc([C@H]2C[C@@H]2c2ccc(F)cc2)c(Cl)c1=O ; Cc1cnc(-c2cc3[nH]ncc3cc2F)cc1-n1c(C)cc([C@H]2C[C@@H]2c2cnn(C(F)F)c2)c(Cl)c1=O]

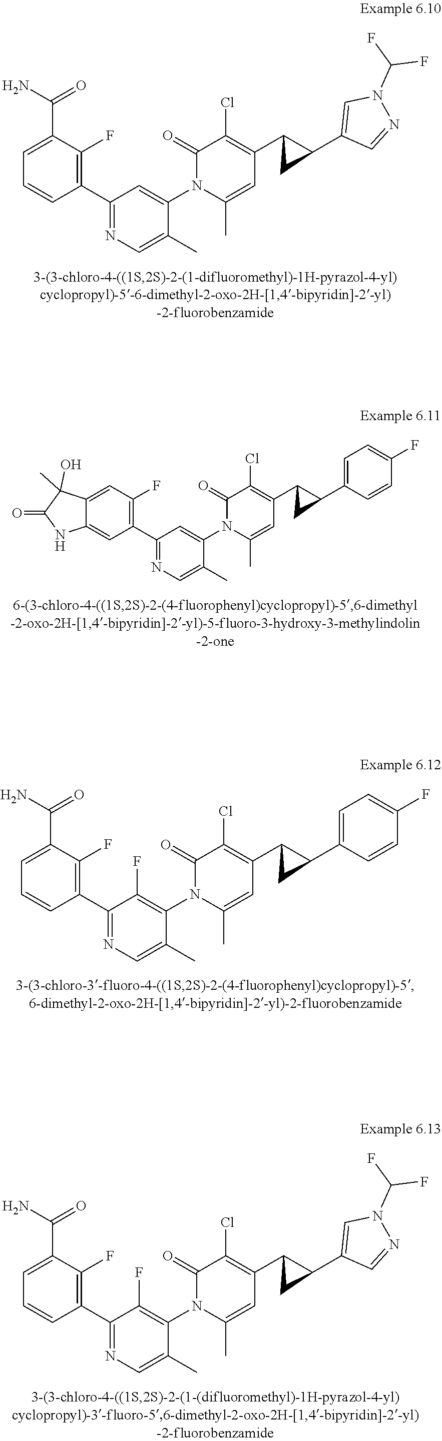 [SMILES: Cc1cnc(-c2cccc(C(N)=O)c2F)cc1-n1c(C)cc([C@H]2C[C@@H]2c2cnn(C(F)F)c2)c(Cl)c1=O ; Cc1cnc(-c2cc3c(cc2F)C(C)(O)C(=O)N3)cc1-n1c(C)cc([C@H]2C[C@@H]2c2ccc(F)cc2)c(Cl)c1=O ; Cc1cnc(-c2cccc(C(N)=O)c2F)c(F)c1-n1c(C)cc([C@H]2C[C@@H]2c2ccc(F)cc2)c(Cl)c1=O ; Cc1cnc(-c2cccc(C(N)=O)c2F)c(F)c1-n1c(C)cc([C@H]2C[C@@H]2c2cnn(C(F)F)c2)c(Cl)c1=O]

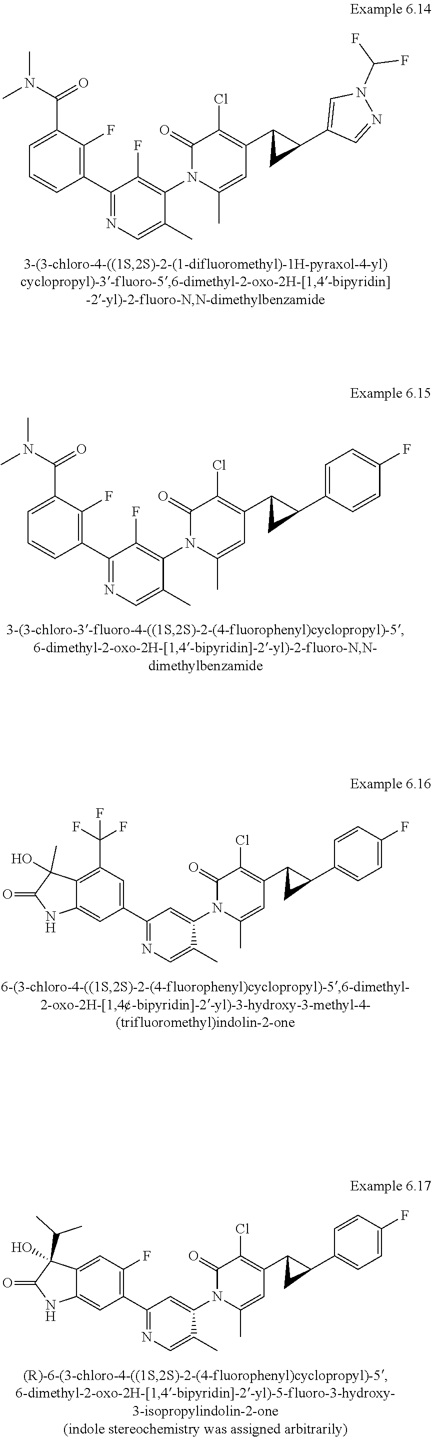

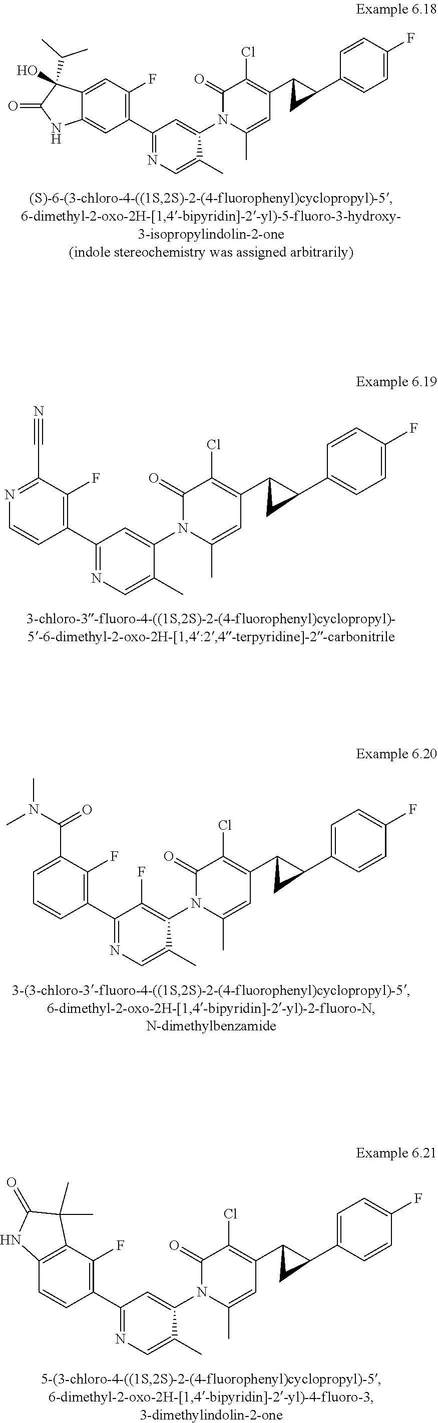

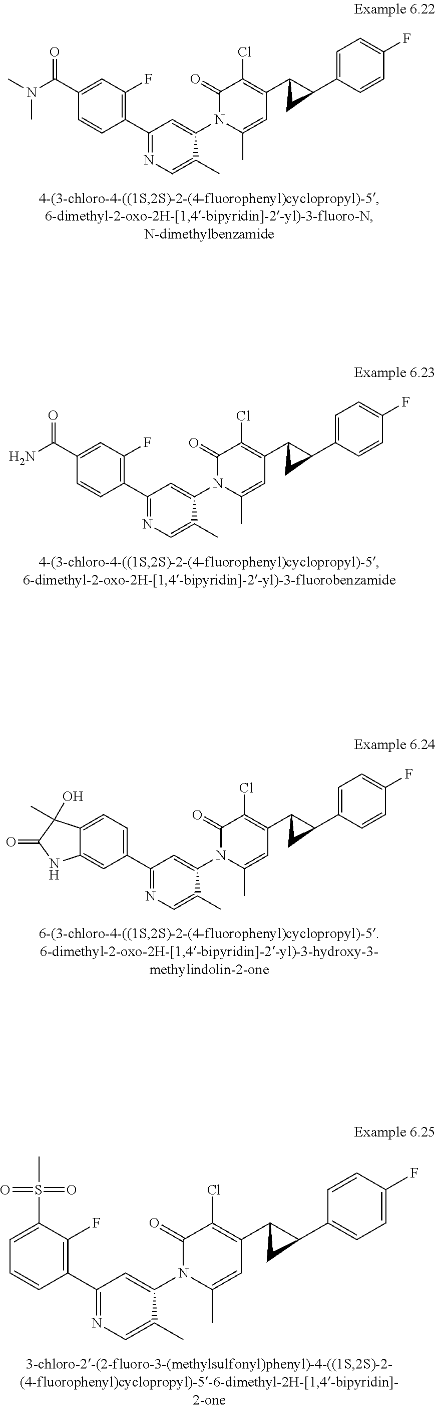

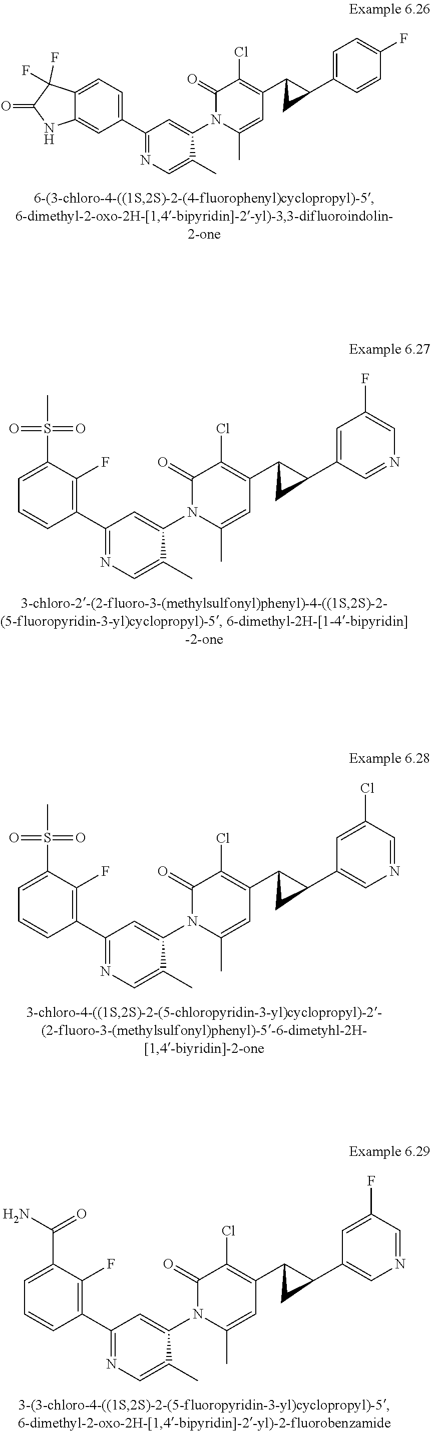

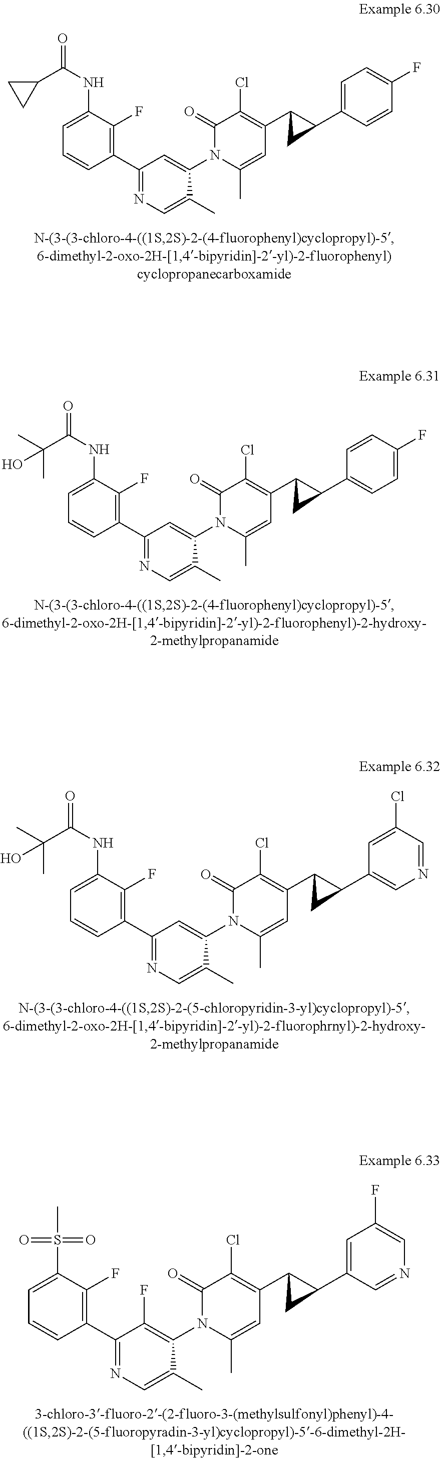

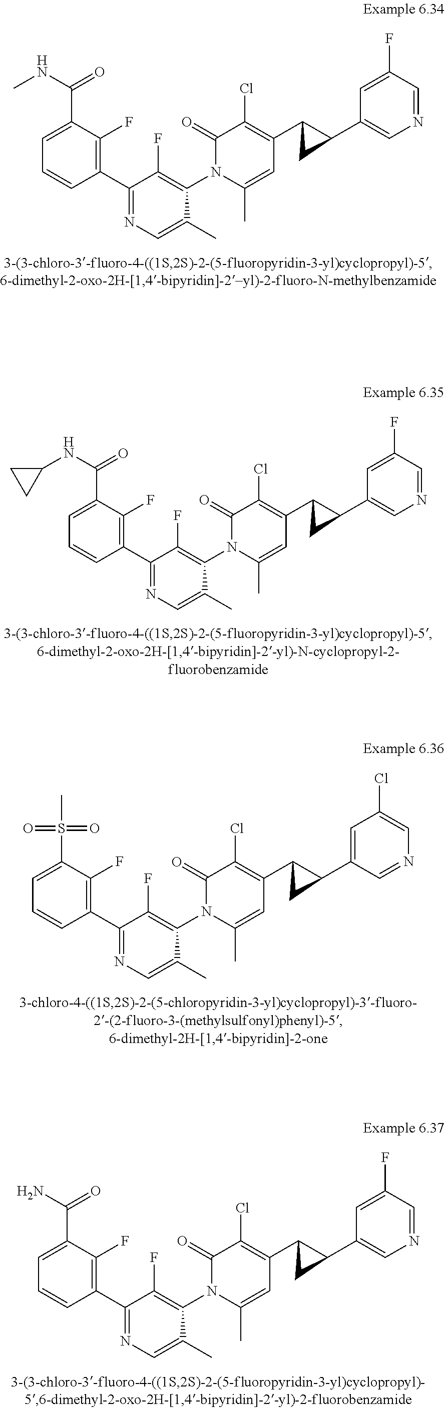

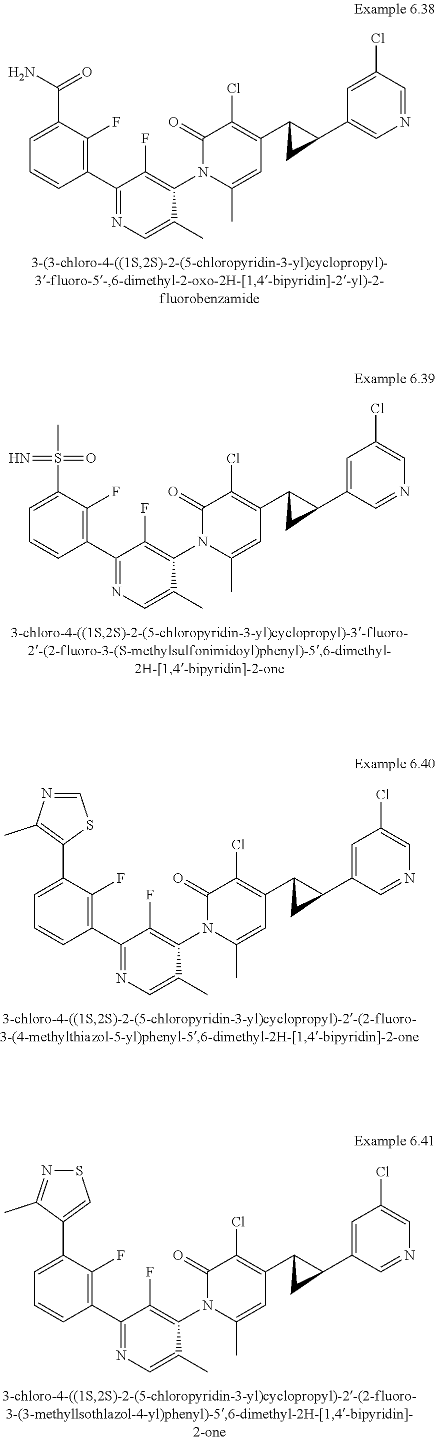

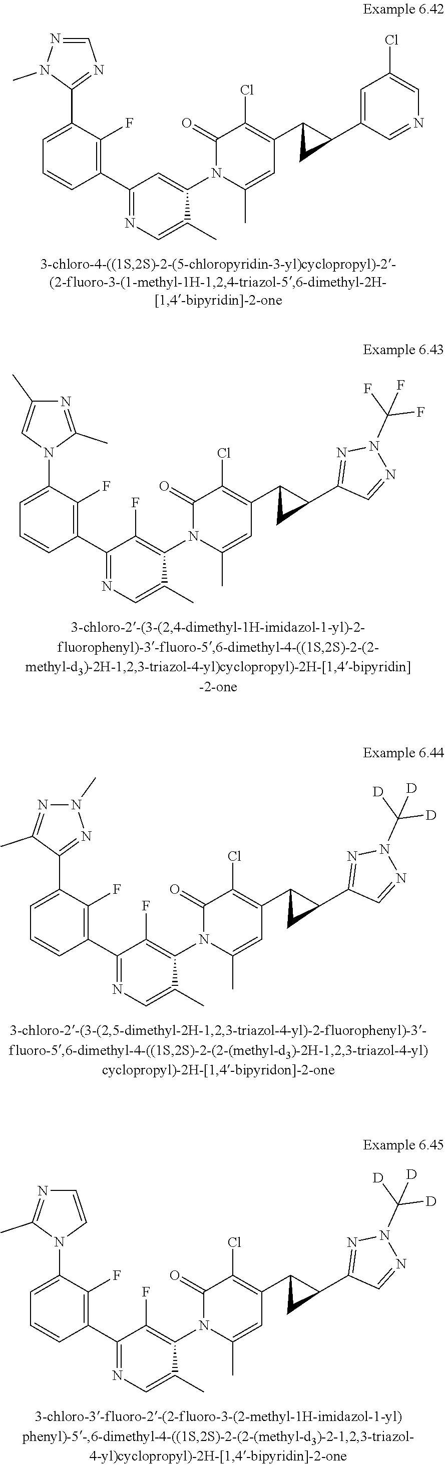

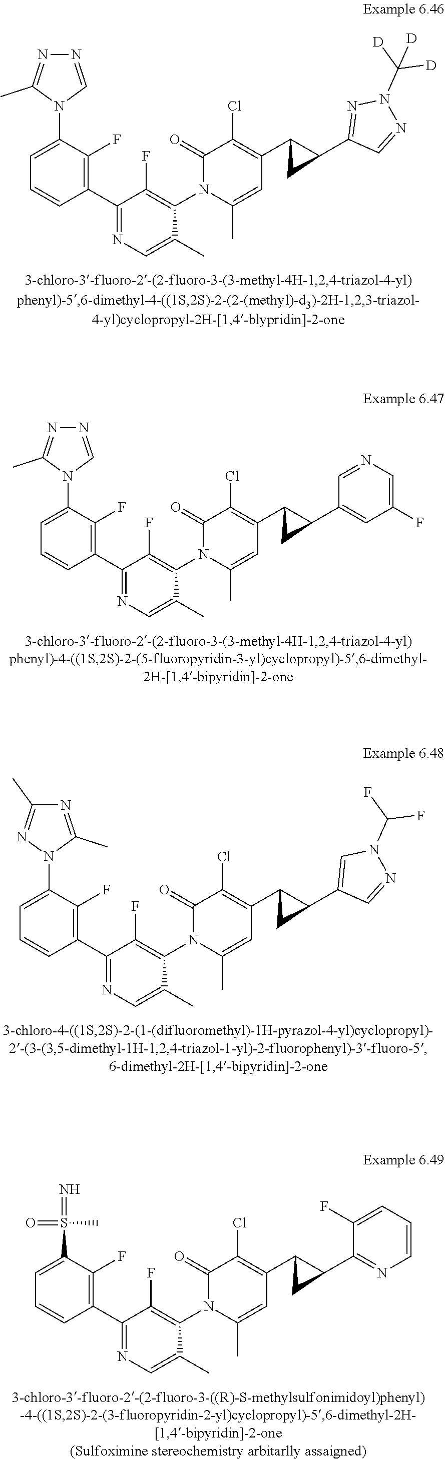

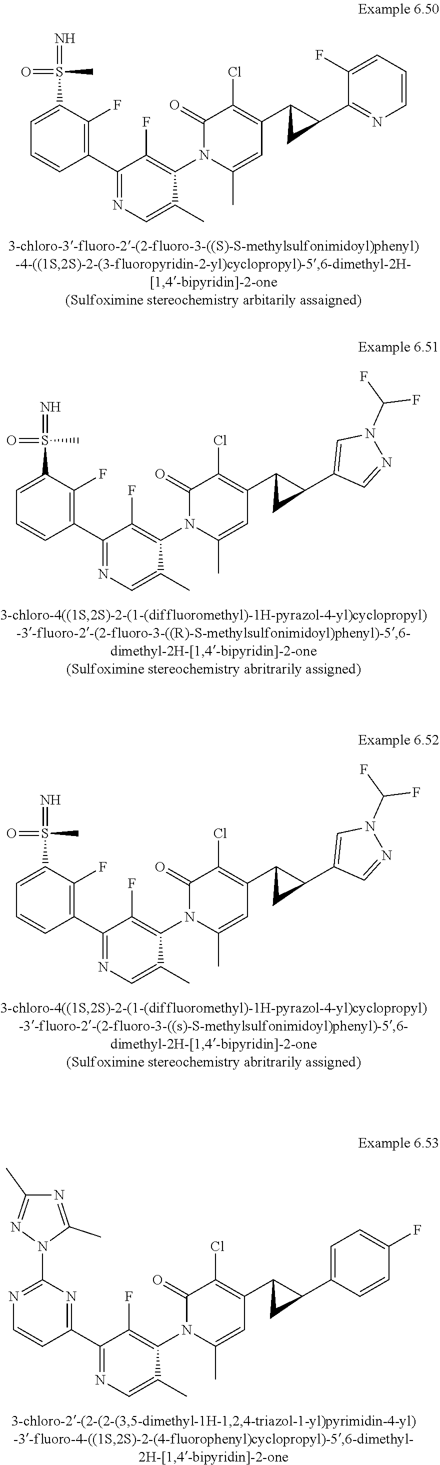

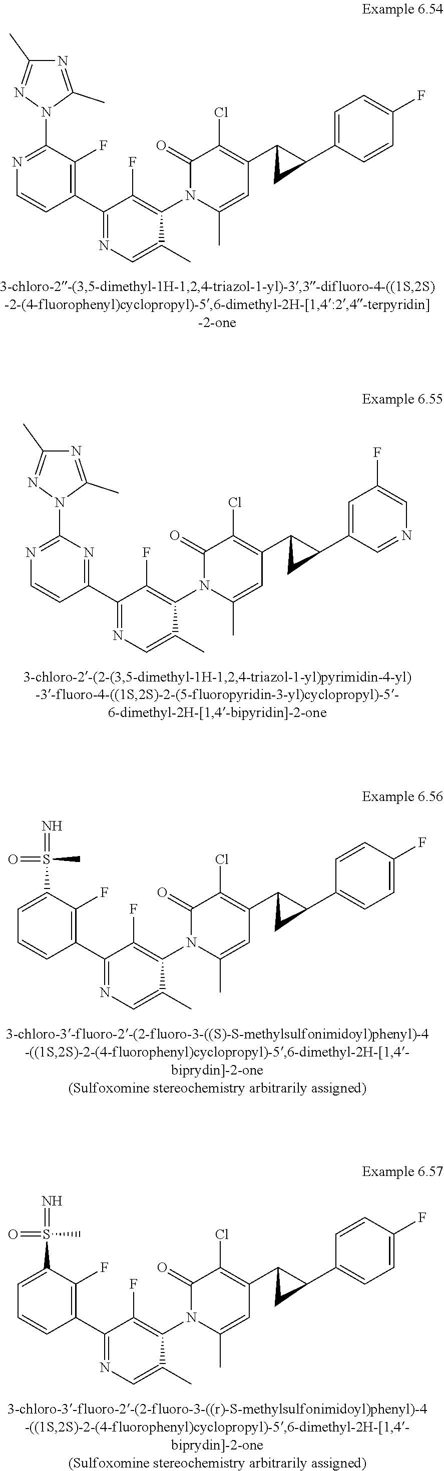

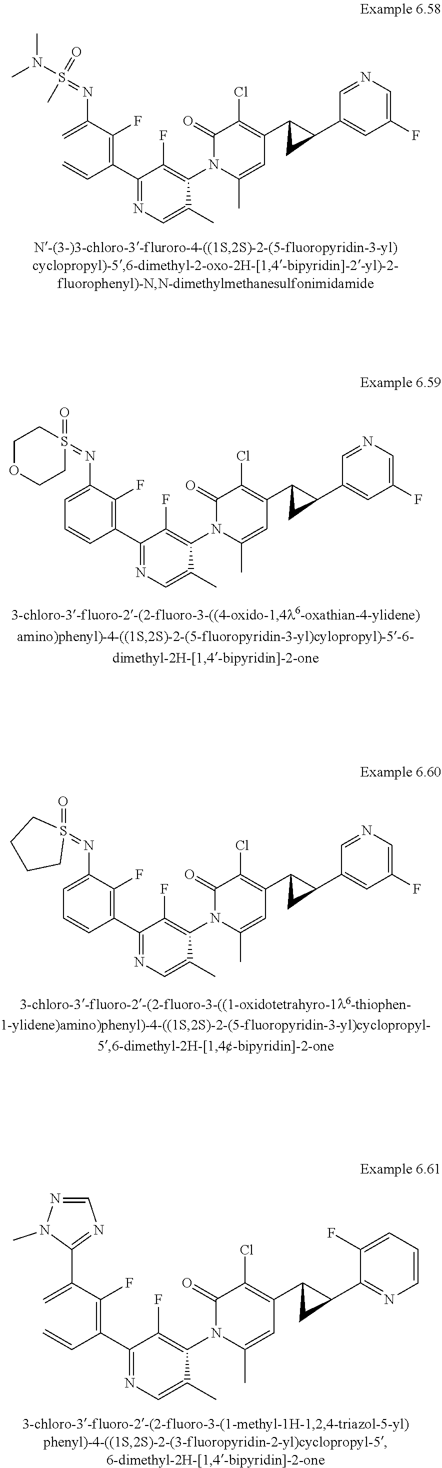

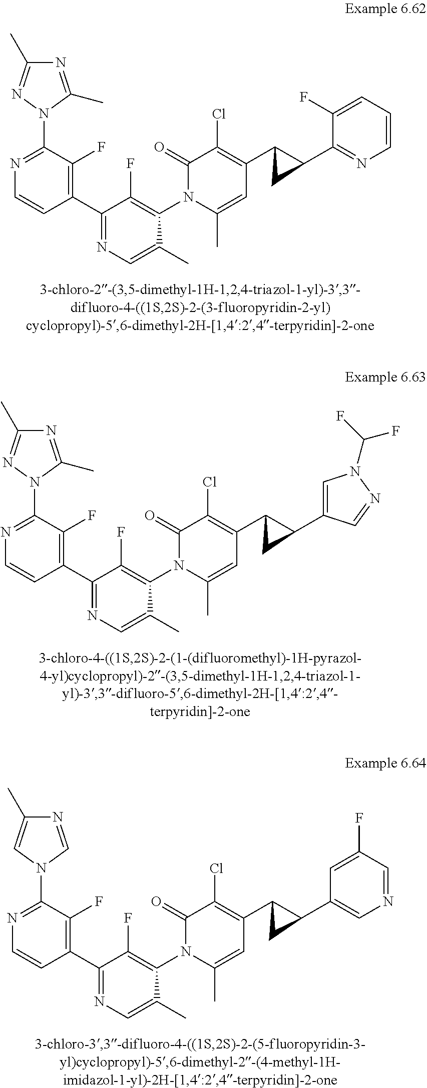

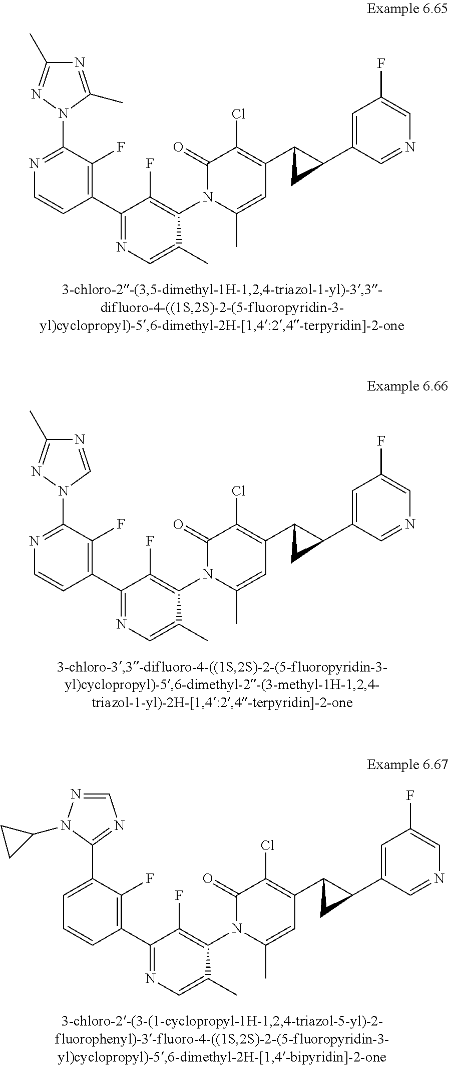

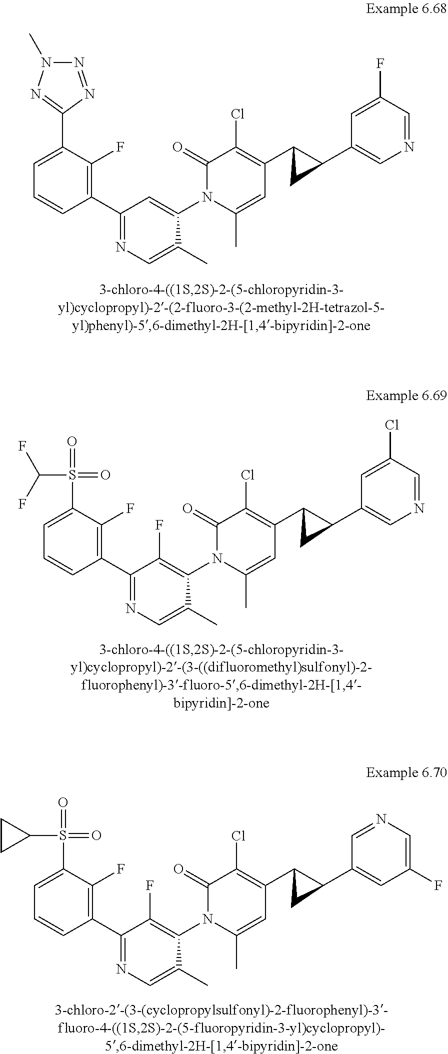

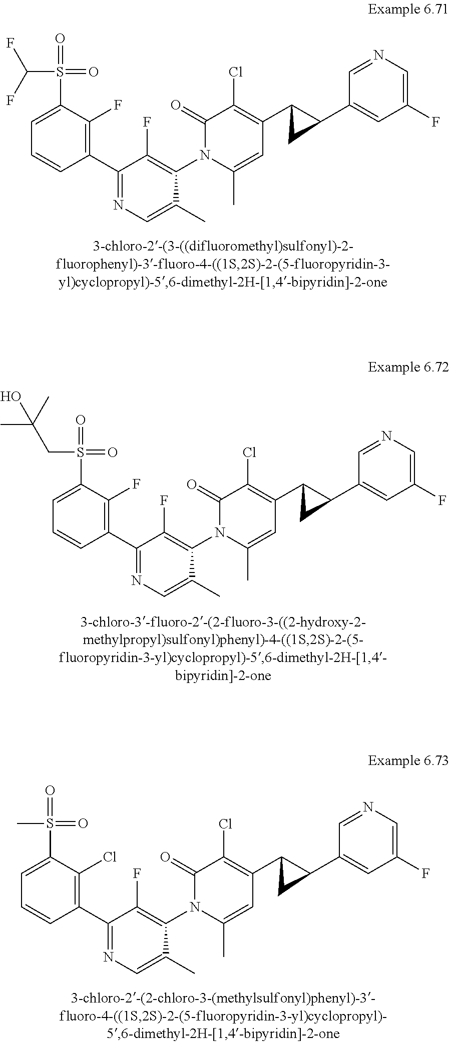

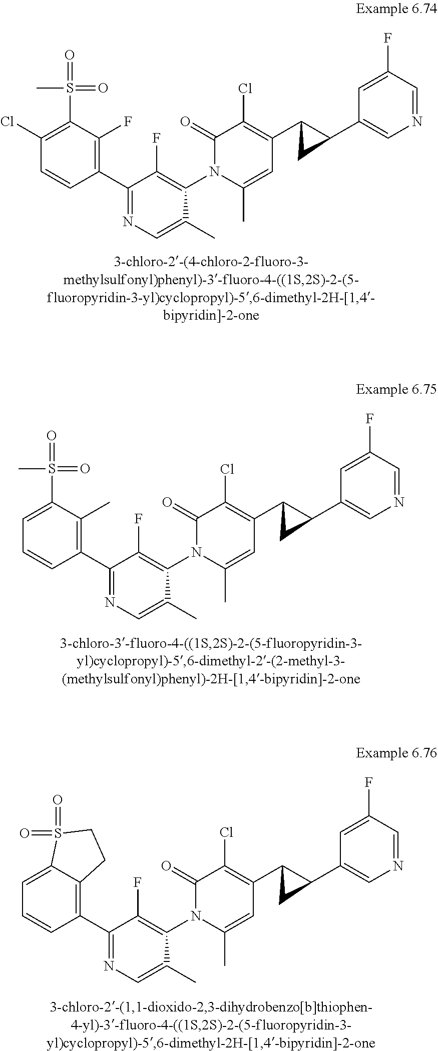

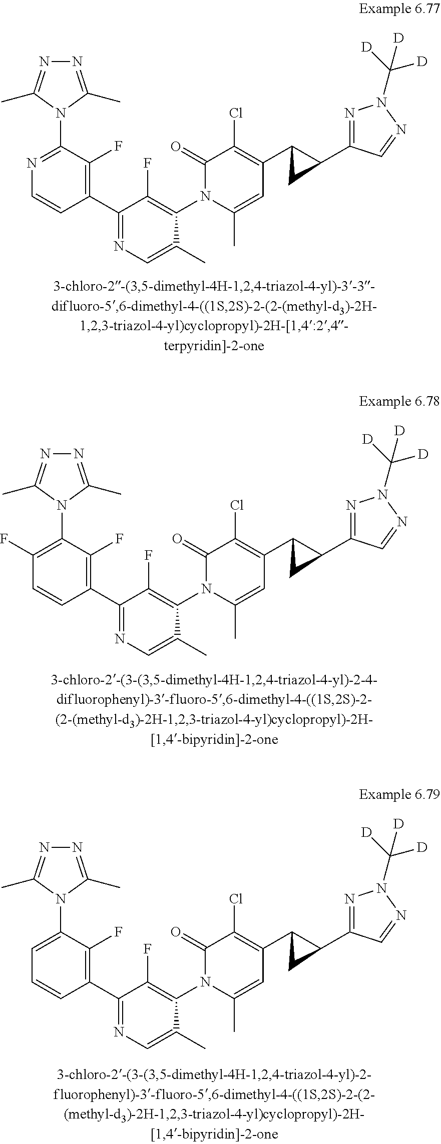

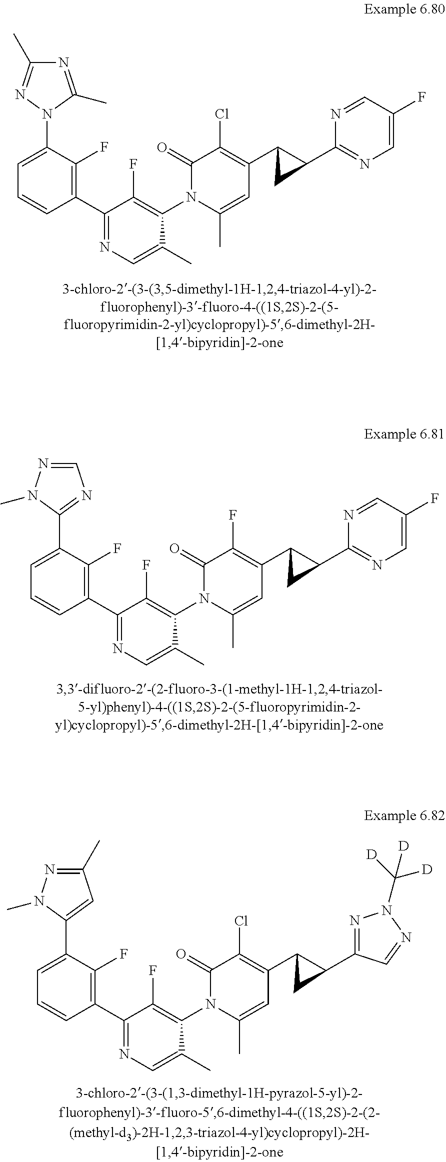

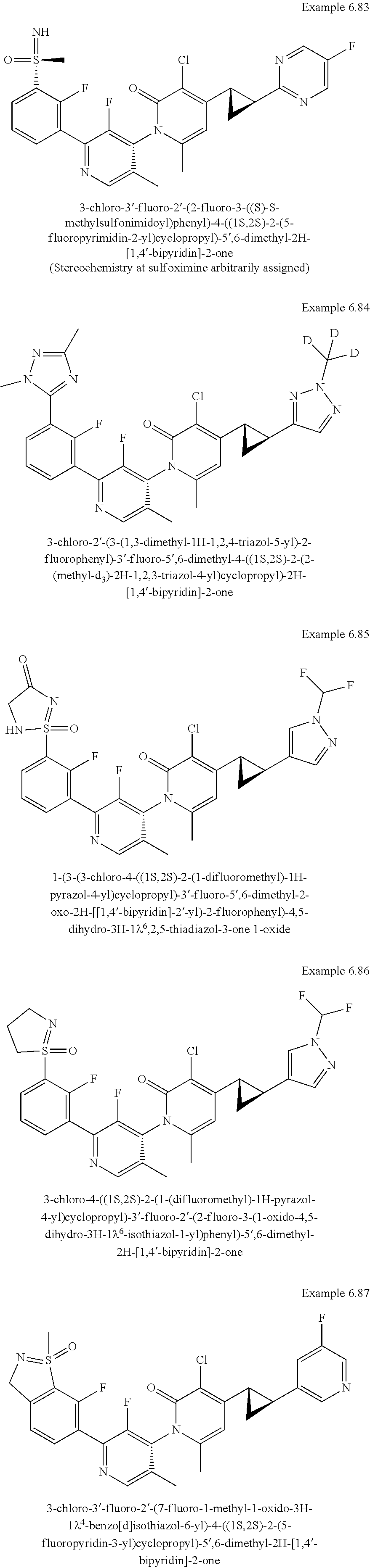

TABLE 5
Procedures analogous to Example 6.1 Synthesis

| Example | Starting | Instead of | |
|---|---|---|---|
| Number | Material | C7.6 | Variation from Example 6.1 |
| 6.2 | D4.1 | C4.1 | Boc-product subjeted to 1:1 TFA/DCM for 2 hr for |
| | | | deprotection |
| 6.3 | D4.1 | C7.12 | |
| 6.4 | (rac)-D4.3 | C1.9 | Pd(dppf)Cl₂ instead of Pd(PPh₃)₄ |
| 6.5 | (rac)-D4.3 | C1.15 | Pd(dppf)Cl₂ instead of Pd(PPh₃)₄ |
| 6.6 | (rac)-D4.1 | C7.1 | Pd SPhos G3 instead of Pd(PPh3)4 |
| 6.7 | (rac)-D4.1 | C7.21 | Pd SPhos G3 instead of Pd(PPh3)4 |
| 6.8 | (rac)-D4.1 | C7.7 | Pd SPhos G3 instead of Pd(PPh3)4 |
| 6.9 | (rac)-D4.3 | C1.12 | Pd(dppf)Cl₂ instead of Pd(PPh₃)₄ |
| 6.10 | (rac)-D4.3 | C1.14 | Pd(dppf)Cl₂ instead of Pd(PPh₃)₄ |
| 6.11 | (rac)-D4.1 | C7.19 | |
| 6.12 | (rac)-D4.2 | C1.14 | Pd(dppf)Cl₂ instead of Pd(PPh₃)₄ |
| 6.13 | (rac)-D4.4 | C1.14 | Pd(dppf)Cl₂ instead of Pd(PPh₃)₄ |
| 6.14 | (rac)-D4.4 | C1.9 | Pd(dppf)Cl₂ instead of Pd(PPh₃)₄ |
| 6.15 | (rac)-D4.2 | C1.9 | Pd(dppf)Cl₂ instead of Pd(PPh₃)₄ |
| 6.16 | D4.1 | C7.17 | SEM-product subjeted to 1:1 TFA/DCM for 18 hr, then |
| | | | for NH₄OH for 1 hr for deprotection |
| 6.17 | D4.1 | C7.16 | SEM-product subjeted to 1:1 TFA/DCM for 18 hr, then |
| | | | for NH₄OH for 1 hr for deprotection; stereoisomers |
| | | | were separated during normal phase chromatography |
| 6.18 | D4.1 | C7.16 | SEM-product subjeted to 1:1 TFA/DCM for 18 hr, then |
| | | | for NH₄OH for 1 hr for deprotection; stereoisomers |
| | | | were separated during normal phase chromatography |
| 6.19 | (rac)-C4.1 | C7.2 | Pd(dppf)Cl₂ instead of Pd(PPh₃)₄ |
| 6.20 | D4.2 | C1.9 | Pd(dppf)Cl₂ instead of Pd(PPh₃)₄ |
| 6.21 | D4.1 | C7.15 | |
| 6.22 | D4.1 | C1.19 | Pd(dppf)Cl₂ instead of Pd(PPh₃)₄ |
| 6.23 | D4.1 | C1.18 | Pd(dppf)Cl₂ instead of Pd(PPh₃)₄ |
| 6.24 | D4.1 | C7.18 | |
| 6.25 | D4.1 | C1.17 | Pd(dppf)Cl₂ instead of Pd(PPh₃)₄ |
| 6.26 | D4.1 | C7.20 | SEM-product subjected to 1:1 TFA/DCM for 18 hr, then |
| | | | for NH₄OH for 1 hr for deprotection |
| 6.27 | D4.7 | C1.17 | Pd(dppf)Cl₂ instead of Pd(PPh₃)₄ |
| 6.28 | D4.5 | C1.17 | Pd(dppf)Cl₂ instead of Pd(PPh₃)₄ |
| 6.29 | D4.7 | C1.14 | Pd(dppf)Cl₂ instead of Pd(PPh₃)₄ |
| 6.30 | D4.1 | C7.13 | |
| 6.31 | D4.1 | C7.11 | |
| 6.32 | D4.7 | C7.11 | |
| 6.33 | D4.8 | C1.17 | Pd(dppf)Cl₂ instead of Pd(PPh₃)₄ |
| 6.34 | D4.8 | C1.13 | Pd(dppf)Cl₂ instead of Pd(PPh₃)₄ |
| 6.35 | D4.8 | C1.21 | Pd(dppf)Cl₂ instead of Pd(PPh₃)₄ |
| 6.36 | D4.6 | C1.17 | Pd(dppf)Cl₂ instead of Pd(PPh₃)₄ |
| 6.37 | D4.8 | C1.14 | Pd(dppf)Cl₂ instead of Pd(PPh₃)₄ |
| 6.38 | D4.6 | C1.14 | Pd(dppf)Cl₂ instead of Pd(PPh₃)₄ |
| 6.39 | D4.6 | C10.1 | Pd(dppf)Cl₂ instead of Pd(PPh₃)₄; treated with |
| | | | DCM/TFA for 15 min after coupling |
| 6.40 | D4.5 | C9.1 | Pd(dppf)Cl₂ instead of Pd(PPh₃)₄ |
| 6.41 | D4.5 | C9.2 | Pd(dppf)Cl₂ instead of Pd(PPh₃)₄ |
| 6.42 | D4.5 | C9.3 | Pd(dppf)Cl₂ instead of Pd(PPh₃)₄ |
| 6.43 | D4.13 | C11.1 | Pd(dppf)Cl₂ instead of Pd(PPh₃)₄ |
| 6.44 | D4.13 | C12.2 | Pd(dppf)Cl₂ instead of Pd(PPh₃)₄ |
| 6.45 | D4.13 | C11.2 | Pd(dppf)Cl₂ instead of Pd(PPh₃)₄ |
| 6.46 | D4.13 | C11.4 | Pd(dppf)Cl₂ instead of Pd(PPh₃)₄ |
| 6.47 | D4.10 | C11.4 | Pd(dppf)Cl₂ instead of Pd(PPh₃)₄ |
| 6.48 | D4.4 | C13.3 | Pd(dppf)Cl₂ instead of Pd(PPh₃)₄ |
| 6.49 | D4.12 | C10.1b | Pd(dppf)Cl₂ instead of Pd(PPh₃)₄; Boc product treated |
| | | | with 1:1 TFA/DCM |
| 6.50 | D4.12 | C10.1a | Pd(dppf)Cl₂ instead of Pd(PPh₃)₄; Boc product treated |
| | | | with 1:1 TFA/DCM |
| 6.51 | D4.4 | C10.1b | Pd(dppf)Cl₂ instead of Pd(PPh₃)₄; Boc product treated |
| | | | with 1:1 TFA/DCM |
| 6.52 | D4.4 | C10.1a | Pd(dppf)Cl₂ instead of Pd(PPh₃)₄; Boc product treated |
| | | | with 1:1 TFA/DCM |
| 6.53 | D4.2 | C7.25 | Pd(dppf)Cl₂ instead of Pd(PPh₃)₄ |
| 6.54 | D4.2 | C7.22 | Pd(dppf)Cl₂ instead of Pd(PPh₃)₄ |
| 6.55 | D4.8 | C7.25 | Pd(dppf)Cl₂ instead of Pd(PPh₃)₄ |
| 6.56 | D4.2 | C10.1b | Pd(dppf)Cl₂ instead of Pd(PPh₃)₄; Boc product treated |
| | | | with 1:1 TFA/DCM |
| 6.57 | D4.2 | C10.1a | Pd(dppf)Cl₂ instead of Pd(PPh₃)₄; Boc product treated |
| | | | with 1:1 TFA/DCM |
| 6.58 | D4.8 | C18.2 | Pd(dppf)Cl₂ instead of Pd(PPh₃)₄ |
| 6.59 | D4.8 | C18.1 | Pd(dppf)Cl₂ instead of Pd(PPh₃)₄ |
| 6.60 | D4.8 | C18.3 | Pd(dppf)Cl₂ instead of Pd(PPh₃)₄ |
| 6.61 | D4.12 | C9.3 | Pd(dppf)Cl₂ instead of Pd(PPh₃)₄ |
| 6.62 | D4.12 | C7.22 | Pd(dppf)Cl₂ instead of Pd(PPh₃)₄ |
| 6.63 | D4.4 | C7.22 | Pd(dppf)Cl₂ instead of Pd(PPh₃)₄ |
| 6.64 | D4.8 | C11.3 | Pd(dppf)Cl₂ instead of Pd(PPh₃)₄ |
| 6.65 | D4.8 | C7.22 | Pd(dppf)Cl₂ instead of Pd(PPh₃)₄ |
| 6.66 | D4.8 | C7.23 | Pd(dppf)Cl₂ instead of Pd(PPh₃)₄ |
| 6.67 | D4.8 | C12.4 | Pd(dppf)Cl₂ instead of Pd(PPh₃)₄ |
| 6.68 | D4.5 | C12.5 | Pd(dppf)Cl₂ instead of Pd(PPh₃)₄ |
| 6.69 | D4.6 | C19.2 | Pd(dppf)Cl₂ instead of Pd(PPh₃)₄ |
| 6.70 | D4.8 | C19.1 | Pd(dppf)Cl₂ instead of Pd(PPh₃)₄ |
| 6.71 | D4.8 | C19.2 | Pd(dppf)Cl₂ instead of Pd(PPh₃)₄ |
| 6.72 | D4.8 | C19.3 | Pd(dppf)Cl₂ instead of Pd(PPh₃)₄ |
| 6.73 | D4.8 | C7.31 | Pd(dppf)Cl₂ instead of Pd(PPh₃)₄ |
| 6.74 | D4.8 | C7.33 | Pd(dppf)Cl₂ instead of Pd(PPh₃)₄ |
| 6.75 | D4.8 | C7.32 | Pd(dppf)Cl₂ instead of Pd(PPh₃)₄ |
| 6.76 | D4.8 | C7.34 | Pd(dppf)Cl₂ instead of Pd(PPh₃)₄ |
| 6.77 | D4.13 | C7.28 | Pd(dppf)Cl₂ instead of Pd(PPh₃)₄ |
| 6.78 | D4.13 | C7.29 | Pd(dppf)Cl₂ instead of Pd(PPh₃)₄ |
| 6.79 | D4.13 | C7.30 | Pd(dppf)Cl₂ instead of Pd(PPh₃)₄ |
| 6.80 | D4.19 | C13.3 | Pd(dppf)Cl₂ instead of Pd(PPh₃)₄ |
| 6.81 | D4.19 | C9.3 | Pd(dppf)Cl₂ instead of Pd(PPh₃)₄ |
| 6.82 | D4.16 | C12.3 | Pd(dppf)Cl₂ instead of Pd(PPh₃)₄ |
| 6.83 | D4.19 | C10.1a | Pd(dppf)Cl₂ instead of Pd(PPh₃)₄; Boc product treated |
| | | | with 1:1 TFA/DCM |
| 6.84 | D4.13 | C12.1 | Pd(dppf)Cl₂ instead of Pd(PPh₃)₄ |
| 6.85 | D4.4 | C20.1 | Pd(dppf)Cl₂ instead of Pd(PPh₃)₄ |
| 6.86 | D4.4 | C20.2 | Pd(dppf)Cl₂ instead of Pd(PPh₃)₄ |
| 6.87 | D4.8 | C20.3 | Pd(dppf)Cl₂ instead of Pd(PPh₃)₄ |

### Example 7.1: N-(3-(3-chloro-4-((1S,2S)-2-(4-fluoro phenyl)cyclopropyl)-5′,6-dimethyl-2-oxo-2H-[1,4′-bipyridin]-2′-yl)-2-fluorophenyl)-3-methyloxetane-3-carboxamide

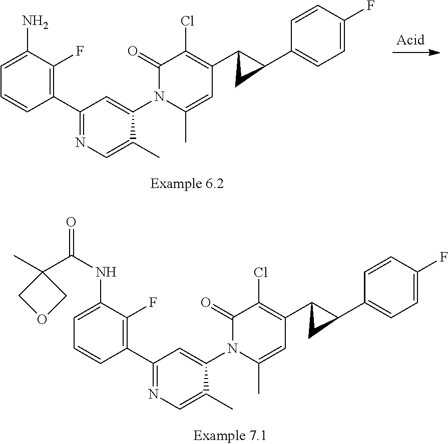

A vial was charged with 1-[2-(3-amino-2-fluoro-phenyl)-5-methyl-4-pyridyl]-3-chloro-4-[(1S,2S)-2-(4-fluorophenyl)cyclopropyl]-6-methyl-pyridin-2-one (Example 6.2) (15 mg, 0.031 mmol), 3-methyloxetane-3-carboxylic acid (Acid) (3.7 mg, 0.031 mmol), 4-dimethylaminopyridine (0.3 mg, 0.003 mmol), and dichloromethane (0.3 mL). To this, EDC (6.6 mg, 0.035 mmol) was added, and the reaction was stirred at ambient temperature for 16 hours. It was concentrated and purified by flash chromatography (SiO₂, hexane-EtOAc) followed by reverse phase HPLC (Silicycle SiliaChrom SB C18, 5 μm, 150A, 30×250 mm; 0.1% TFA in MeCN-0.1% TFA in water) to give N-(3-(3-chloro-4-((1S,2S)-2-(4-fluorophenyl)cyclopropyl)-5′,6-dimethyl-2-oxo-2H-[1,4′-bipyridin]-2′-yl)-2-fluorophenyl)-3-methyloxetane-3-carboxamide (Example 7.1).

The following compounds in Table 6 were prepared analogously to Example 7.1 modified as described:

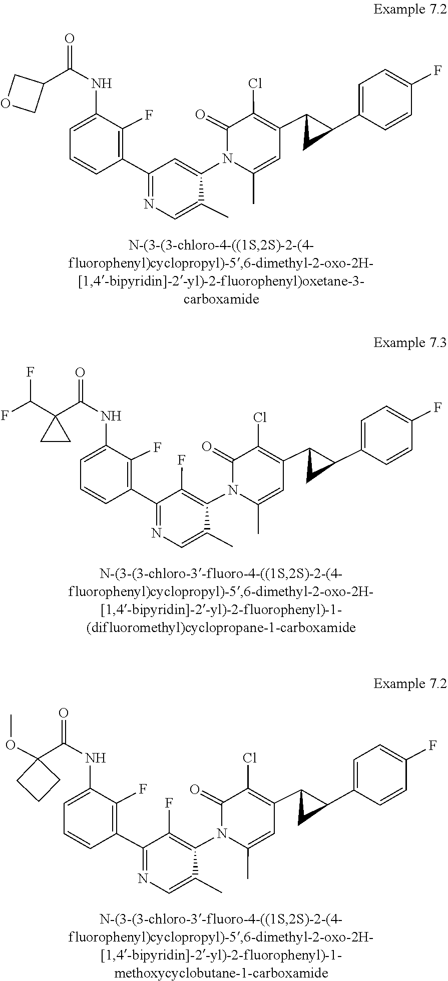

TABLE 6
Procedures analogous to Example 7.1 Synthesis

| Example | |
|---|---|
| Number | Acid |
| 7.2 | oxetane-3-carboxylic acid |
| 7.3 | 1-(difluoromethyl)cyclopropane-1-carboxylic acid |
| 7.4 | 1-methoxycyclobutane-1-carboxylic acid |

### Example 8.1: N-(3-(3-chloro-4-((1S,2S)-2-(4-fluoro phenyl)cyclopropyl)-5′,6-dimethyl-2-oxo-2H-[1,4′-bipyridin]-2′-yl)-2-fluorophenyl)-1-fluorocyclopropane-1-carboxamide

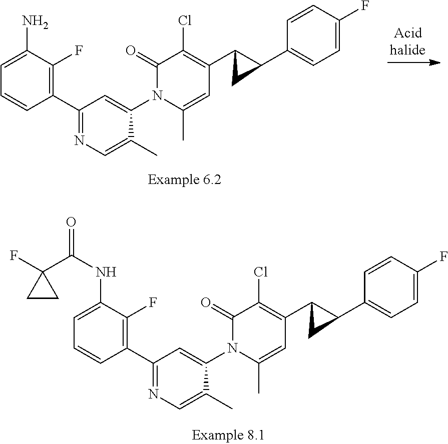

A vial was charged with 2′-(3-amino-2-fluorophenyl)-3-chloro-4-((1S,2S)-2-(4-fluorophenyl)cyclopropyl)-5′,6-dimethyl-2H-[1,4′-bipyridin]-2-one (Example 6.2) (10 mg, 0.021 mmol), DCM (0.2 mL), and pyridine (8.3 mg, 0.11 mmol), followed by 1-fluorocyclopropane-1-carbonyl chloride (3.1 mg, 0.025 mmol). The mixture stirred at room temperature for 18 hours. The mixture was concentrated and subjected to flash chromatography (SiO₂, hexane-EtOAc) followed by reverse phase HPLC (Silicycle SiliaChrom SB C18, 5 μm, 150 Å, 30×250 mm; 0.1% TFA in MeCN-0.1% TFA in water) to give N-(3-(3-chloro-4-((1S,2S)-2-(4-fluorophenyl)cyclopropyl)-5′,6-dimethyl-2-oxo-2H-[1,4′-bipyridin]-2′-yl)-2-fluorophenyl)-1-fluorocyclopropane-1-carboxamide (Example 8.1).

The following compounds in Table 7 were prepared analogously to Example 8.1 modified as described:

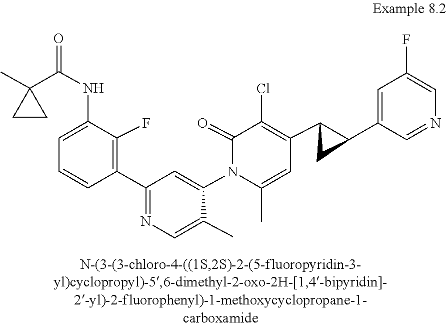

TABLE 7
Procedures analogous to Example 8.1 Synthesis

| Example | Starting | |
|---|---|---|
| Number | Material | Acid halide |
| 8.2 | D5.15 | 1-methylcyclopropane-1-carbonyl chloride |

### Example 9.1: 3-(3-chloro-4-((1S,2S)-2-(cyclopropylcarbamoyl)cyclopropyl)-5′,6-dimethyl-2-oxo-2H-[1,4′-bipyridin]-2′-yl)-2-fluoro-N,N-dimethylbenzamide

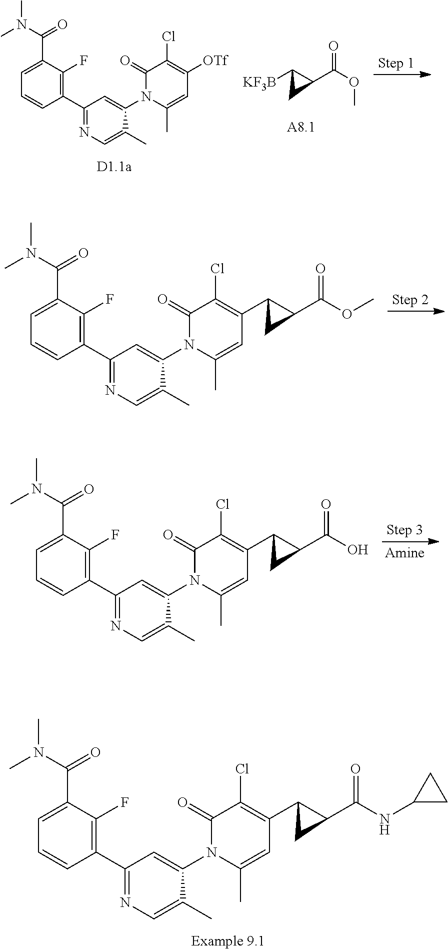

Step 1. A flask was charged with 3-chloro-2′-(3-(dimethylcarbamoyl)-2-fluorophenyl)-5′,6-dimethyl-2-oxo-2H-[1,4′-bipyridin]-4-yl trifluoromethanesulfonate (D1.1a) (1.61 g, 2.93 mmol), methyl (1S,2S)-2-(trifluoro-14-boraneyl)cyclopropane-1-carboxylate potassium salt (A8.1) (1.21 g, 5.86 mmol), Pd(dppf)Cl₂-DCM (239 mg, 0.29 mmol), Cs₂CO₃ (2.87 g, 8.79 mmol), PhMe (40 mL), and water (8 mL). The mixture was heated to 90° C. for 2 hr. The reaction was allowed to cool to room temperature, after which water (20 mL) was added and the mixture was extracted with EtOAc (3×20 mL). The combined organics were dried over Na₂SO₄, filtered, and concentrated. The residue was subjected to flash chromatography (SiO₂, hexane-EtOAc-MeOH) to give methyl (1S,2S)-2-(3-chloro-2′-(3-(dimethylcarbamoyl)-2-fluorophenyl)-5′,6-dimethyl-2-oxo-2H-[1,4′-bipyridin]-4-yl)cyclopropane-1-carboxylate.

Step 2. A flask was charged with (1S,2S)-2-(3-chloro-2′-(3-(dimethylcarbamoyl)-2-fluorophenyl)-5′,6-dimethyl-2-oxo-2H-[1,4′-bipyridin]-4-yl)cyclopropane-1-carboxylate (1.45 g, 2.91 mmol), LiOH (387 mg, 16.2 mmol), THF (10 mL), MeOH (10 mL), and water (5 mL). The mixture was allowed to stir for 1 hr, after which HCl (1M, 3 mL) was added. The mixture was extracted with EtOAc (6×10 mL). The combined organics were dried over Na₂SO₄, filtered, and concentrated to afford crude (1S,2S)-2-(3-chloro-2′-(3-(dimethylcarbamoyl)-2-fluorophenyl)-5′,6-dimethyl-2-oxo-2H-[1,4′-bipyridin]-4-yl)cyclopropane-1-carboxylic acid, which was used without further purification.

Step 3. A vial was charged (1S,2S)-2-(3-chloro-2′-(3-(dimethylcarbamoyl)-2-fluorophenyl)-5′,6-dimethyl-2-oxo-2H-[1,4′-bipyridin]-4-yl)cyclopropane-1-carboxylic acid (20 mg, 0.041 mmol), cyclopropylamine (3.5 mg, 0.062 mmol), HATU (14.6 mg, 0.062 mmol), MeCN (0.5 mL), followed by iPr₂EtN (0.036 mL, 0.21 mmol). The mixture stirred for 18 his at room temperature. Water (1 mL) was added, and the mixture was extracted with EtOAc (3×1 mL). The combined organics were dried over Na₂SO₄, filtered, and concentrated. The residue was subjected to reverse phase HPLC (Silicycle SiliaChrom SB C18, 5 μm, 150A, 30×250 mm; 0.1% TFA in MeCN-0.1% TFA in water) to give 3-(3-chloro-4-((1S,2S)-2-(cyclopropylcarbamoyl)cyclopropyl)-5′,6-dimethyl-2-oxo-2H-[1,4′-bipyridin]-2′-yl)-2-fluoro-N,N-dimethylbenzamide (Example 9.1).

The following compounds in Table 8 were prepared analogously to Example 9.1 modified as described:

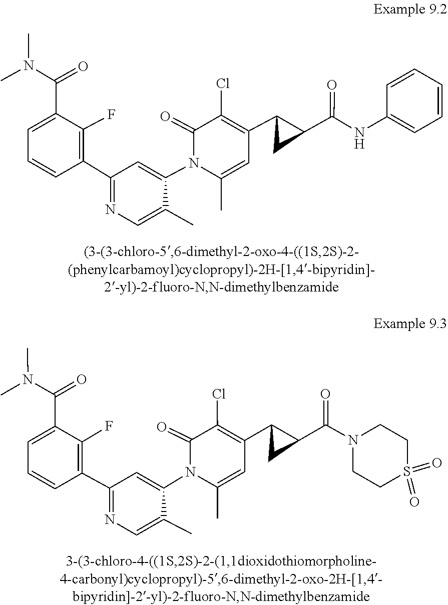

TABLE 8
Procedures analogous to Example 9.1 Synthesis

| Example | |
|---|---|
| Number | Amine |
| 9.2 | aniline |
| 9.3 | thiomorpholine 1,1-dioxide |

### Example 10.1: 2″-(3-(aminomethyl)-3-hydroxyazelidin-1-yl)-3-chloro-3″-fluoro-4-((1S,2S)-2-(4-fluorophenyl)cyclopropyl)-5′,6-dimethyl-2H-[1,4′:2′,4″-terpyridin]-2-one

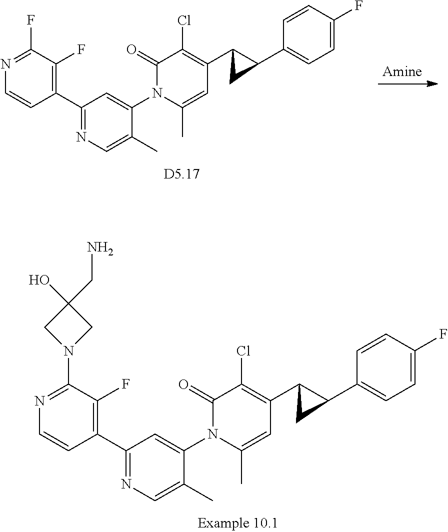 [SMILES: Cc1cnc(-c2ccnc(F)c2F)cc1-n1c(C)cc([C@H]2C[C@@H]2c2ccc(F)cc2)c(Cl)c1=O ; Cc1cnc(-c2ccnc(N3CC(O)(CN)C3)c2F)cc1-n1c(C)cc([C@H]2C[C@@H]2c2ccc(F)cc2)c(Cl)c1=O]

A vial was charged with 3-chloro-2″,3″-difluoro-4-((1S,2S)-2-(4-fluorophenyl)cyclopropyl)-5′,6-dimethyl-2H-[1,4′:2′,4″-terpyridin]-2-one (D5.17) (21 mg, 0.044 mmol), tert-butyl ((3-hydroxyazetidin-3-yl)methyl)carbamate (17.6 mg, 0.087 mmol), K₂CO₃ (18.1 mg, 0.13 mmol), and MeCN (1 mL). The mixture as heated to 75° C. for 18 hrs. Water (1 mL) was added and the mixture was extracted with EtOAc (3×1 mL). The combined organics were dried over Na₂SO₄, filtered, and concentrated. To the residue, DCM (0.5 mL) and TFA (0.5 mL) was added. The mixture was stirred for 1 hr, concentrated, and subjected to reverse phase HPLC (Silicycle SiliaChrom SB C18, 5 μm, 150 Å, 30×250 mm; 0.1% TFA in MeCN-0.10% TFA in water) to give 2″-(3-(aminomethyl)-3-hydroxyazetidin-1-yl)-3-chloro-3″-fluoro-4-((1S,2S)-2-(4-fluorophenyl)cyclopropyl)-5′,6-dimethyl-2H-[1,4′:2′,4″-terpyridin]-2-one (Example 10.1).

The following compounds in Table 9 were prepared analogously to Example 10.1 modified as described:

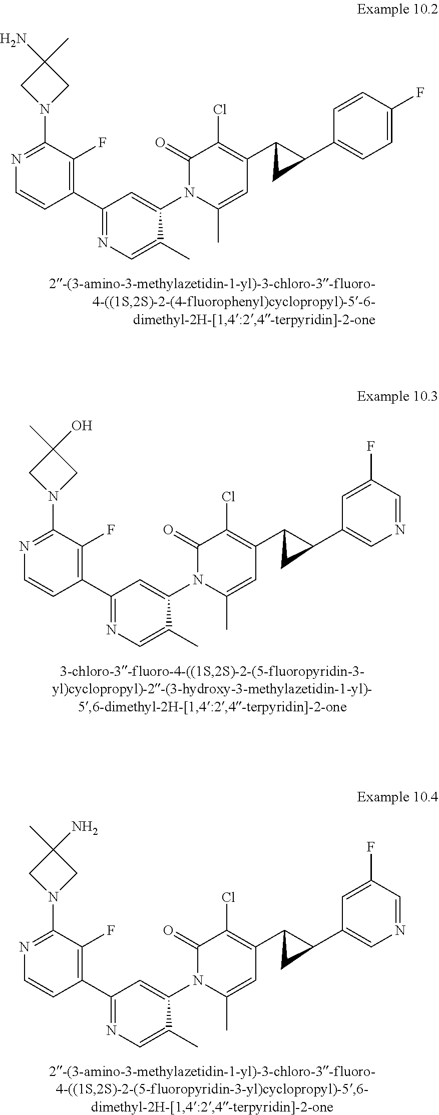

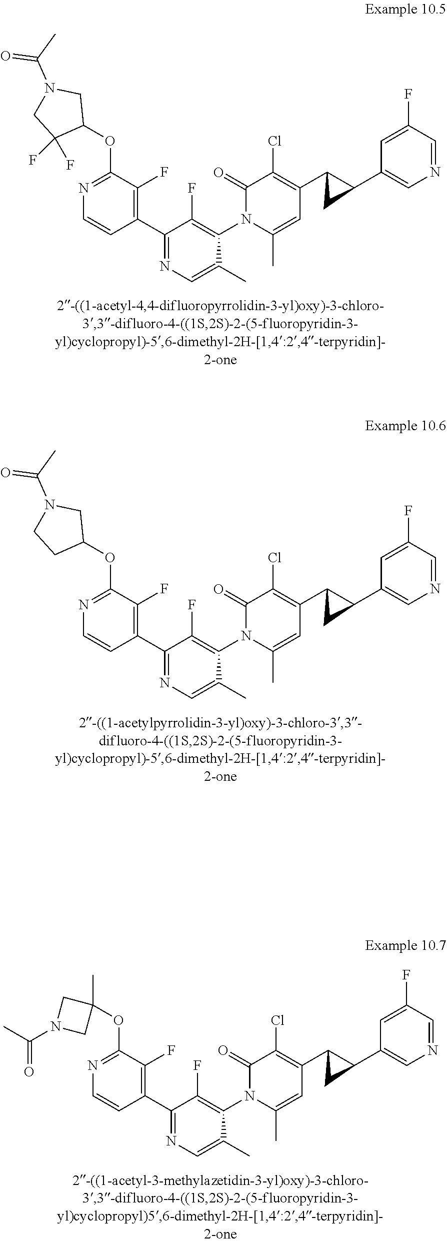

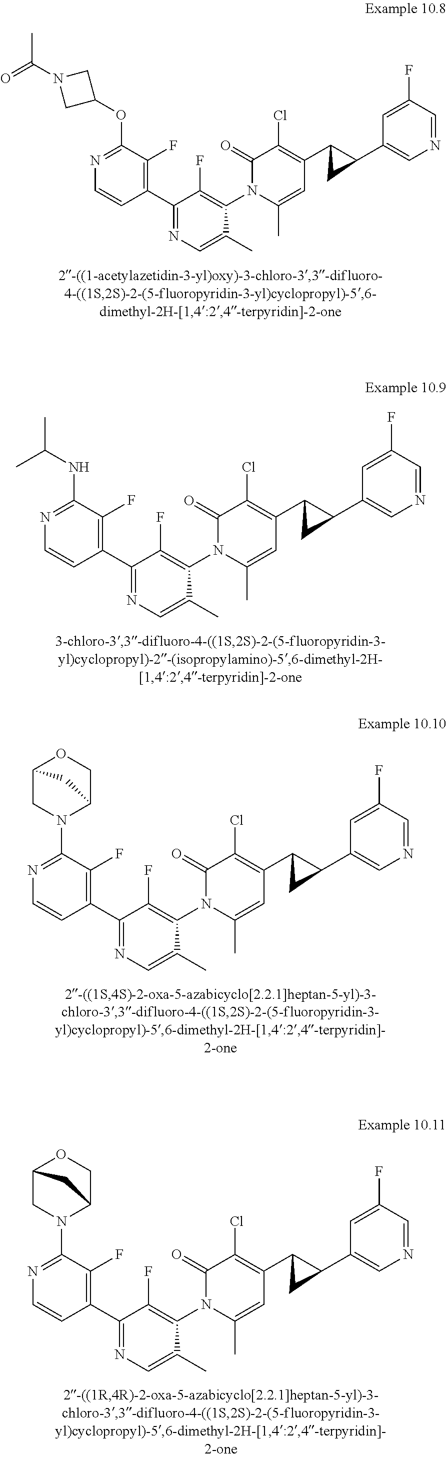

TABLE 9
Procedures analogous to Example 10.1 Synthesis

| Example | Starting | | |
|---|---|---|---|
| Number | Material | Amine | Variation from Example 10.1 |
| 10.2 | D5.17 | tert-butyl (3-methylazetidin- | |
| | | 3-yl)carbamate | |
| 10.3 | D5.18 | 3-methylazetidin-3-ol | Did not treat with DCM/TFA |
| 10.4 | D5.18 | tert-butyl (3-methylazetidin- | |
| | | 3-yl)carbamate | |
| 10.5 | D5.19 | 1-(3,3-difluoro-4- | NaH was used instead of K2CO3, |
| | | hydroxypyrrolidin-1- | did not treat with DCM/TFA |
| | | yl)ethan-1-one | |
| 10.6 | D5.19 | 1-(3-hydroxypyrrolidin-1- | NaH was used instead of K2CO3, |
| | | yl)ethan-1-one | did not treat with DCM/TFA |
| 10.7 | D5.19 | 1-(3-hydroxy-3- | NaH was used instead of K2CO3, |
| | | methylazetidin-1-yl)ethan-1- | did not treat with DCM/TFA |
| | | one | |
| 10.8 | D5.19 | 1-(3-hydroxyazetidin-1- | NaH was used instead of K2CO3, |
| | | yl)ethan-1-one | did not treat with DCM/TFA |
| 10.9 | D5.19 | propan-2-amine | Did not treat with DCM/TFA |
| 10.10 | D5.19 | (1S,4S)-2-oxa-5- | Did not treat with DCM/TFA |
| | | azabicyclo[2.2.1]heptane | |
| 10.11 | D5.19 | (1R,4R)-2-oxa-5- | Did not treat with DCM/TFA |
| | | azabicyclo[2.2.1]heptane | |

### Example 11.1: N-(3-chloro-3″-fluoro-4-((1S,2S)-2-(4-fluorophenyl)cyclopropyl)-5′,6-dimethyl-2-oxo-2H-[1,4′:2′,4″-terpyridin]-2″-yl)-1-methylcyclopropane-1-carboxamide

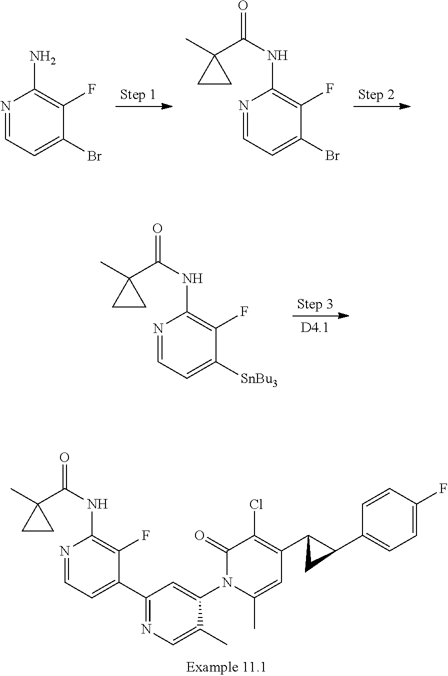

Step 1. To a solution of 4-bromo-3-fluoro-pyridin-2-amine (300 mg, 1.6 mmol) in DCM (6 mL) was added N,N-diisopropylethylamine (0.54 mL, 3.1 mmol) and 1-methylcyclopropanecarbonyl chloride (370 mg, 3.1 mmol). The mixture was allowed to stir at ambient temperature for 24 h. Citric acid (10% aqueous, 5 mL) and the aqueous layer was extracted with DCM (2×5 mL). The combined organic layers were dried over Na₂SO₄, filtered and concentrated. The residue was purified by flash column chromatography (SiO₂, hexanes-EtOAc) to give N-(4-bromo-3-fluoro-2-pyridyl)-1-methyl-cyclopropanecarboxamide. ES/MS: m/z 272.8 [M+H]⁺.

Step 2. A vial was charged with N-(4-bromo-3-fluoro-2-pyridyl)-1-methyl-cyclopropanecarboxamide (68 mg, 0.25 mmol), hexa-n-butylditin (0.14 mL, 0.27 mmol), Pd(OAc)₂ (2.8 mg, 0.012 mmol), tricyclohexylphosphine (7.0 mg, 0.025 mmol) and 1,4-dioxane (0.32 mL). The mixture was sparged with nitrogen for 5 min and sealed in a vial and stirred at 110° C. for 24 hr. The cooled mixture was filtered through Celite, washing with EtoAc. The filtrate was concentrated to give N-(3-fluoro-4-tributylstannyl-2-pyridyl)-1-methyl-cyclopropanecarboxamide, which was used without further purification. ES/MS: m/z 484.4 [M+H]⁺.

Step 3. A vial was charged with crude N-(3-fluoro-4-tributylstannyl-2-pyridyl)-1-methyl-cyclopropanecarboxamide (26 mg, 0.055 mmol), 2′,3-dichloro-4-((1S,2S)-2-(4-fluorophenyl)cyclopropyl)-5′,6-dimethyl-2H-[1,4′-bipyridin]-2-one (D4.1) (20 mg, 0.050 mmol), PdCl₂ (0.90 mg, 0.0050 mmol), CuI (1.9 mg, 0.010 mmol), CsF (15 mg, 0.10 mmol), and PCy₃ (2.8 mg, 0.010 mmol), followed by DMF (0.25 mL). The mixture was sparged with nitrogen, and was then heated to 110° C. for 2 hr. The mixture was concentrated and purified by reverse phase HPLC (Silicycle SiliaChrom SB C18, 5 μm, 150 Å, 30×250 mm; 0.1% TFA in MeCN-0.1% TFA in water) to give N-(3-chloro-3″-fluoro-4-((1S,2S)-2-(4-fluorophenyl)cyclopropyl)-5′,6-dimethyl-2-oxo-2H-[1,4′:2′,4″-terpyridin]-2″-yl)-1-methylcyclopropane-1-carboxamide (Example 11.1).

### Example 12.1: 2-fluoro-3-(4-((1S,2S)-2-(4-fluorophenyl)cyclopropyl)-5′,6-dimethyl-2-oxo-2H-[1,4′-bipyridin]-2′-yl)-N,N-dimethylbenzamide

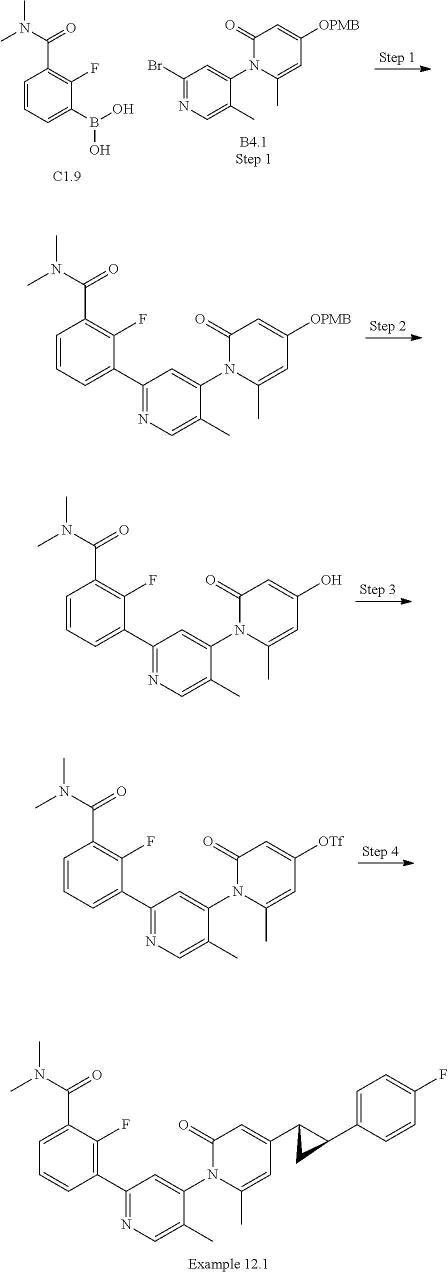

Step 1. To a vial was added (3-(dimethylcarbamoyl)-2-fluorophenyl)boronic acid (63 mg, 0.297 mmol), 2′-bromo-4-((4-methoxybenzyl)oxy)-5′,6-dimethyl-2H-[1,4′-bipyridin]-2-one (100 mg, 0.27 mmol), Pd(dppf)Cl₂ (101 mg, 0.0135 mmol), and cesium carbonate (176 mg, 0.539 mmol). Dioxane (1.6 mL) and water (0.4 mL) were then charged to the vial and the mixture was degassed by sparging with argon. The mixture was then stirred at 80° C. for 4.5 hours. The mixture was cooled to room temperature, dried over sodium sulfate, filtered over celite, and concentrated under reduced pressure. The residue was subjected to flash column chromatography (SiO₂, hexanes-EtOAc) to afford 2-fluoro-3-(4-((4-methoxybenzyl)oxy)-5′,6-dimethyl-2-oxo-2H-[1,4′-bipyridin]-2′-yl)-N,N-dimethylbenzamide.

Step 2. 2-fluoro-3-(4-((4-methoxybenzyl)oxy)-5′,6-dimethyl-2-oxo-2H-[1,4′-bipyridin]-2′-yl)-N,N-dimethylbenzamide (135 mg, 0.27 mmol) was dissolved in DCM (3 mL) and then TFA (0.309 mL, 4.04 mmol) was added. The mixture was stirred at room temperature for 135 minutes then concentrated under reduced pressure. The residue was subjected to flash column chromatography (SiO₂, hexanes-EtOAc, then DCM-MeOH) to afford 2-fluoro-3-(4-hydroxy-5′,6-dimethyl-2-oxo-2H-[1,4′-bipyridin]-2′-yl)-N,N-dimethylbenzamide.

Step 3. To a vial was added solid 2-fluoro-3-(4-hydroxy-5′,6-dimethyl-2-oxo-2H-[1,4′-bipyridin]-2′-yl)-N,N-dimethylbenzamide (20 mg, 0.052 mmol) and then DMF (1 mL). The suspension was stirred and triethylamine (0.022 mL, 0.16 mmol) was added followed by N-phenylbis(trifluoromethane)sulfonimide (23 mg, 0.063 mmol). The mixture was stirred at room temperature for 80 minutes and was then diluted with water (5 mL) and extracted with EtOAc (2×5 mL). The combined organics were dried over Na₂SO₄, filtered, and concentrated under reduced pressure to give crude 2′-(3-(dimethylcarbamoyl)-2-fluorophenyl)-5′,6-dimethyl-2-oxo-2H-[1,4′-bipyridin]-4-yl trifluoromethanesulfonate.

Step 4. Crude 2′-(3-(dimethylcarbamoyl)-2-fluorophenyl)-5′,6-dimethyl-2-oxo-2H-[1,4′-bipyridin]-4-yl trifluoromethanesulfonate (17 mg, 0.032 mmol), trifluoro((1S,2S)-2-(4-fluorophenyl)cyclopropyl)-λ⁴-borane, potassium salt (17 mg, 0.068 mmol), Pd(dppf)Cl₂ (3.9 mg, 0.0052 mmol), and cesium carbonate (51 mg, 0.16 mmol) were added to a vial. PhMe (0.83 mL) and water (0.17 mL) were then charged to the vial and the mixture was degassed by sparging with argon. The mixture was then stirred at 80° C. for 0.5 hours. The mixture was cooled to room temperature, dried over sodium sulfate, filtered over celite, and concentrated under reduced pressure. The residue was then subjected to reverse phase HPLC (Silicycle SiliaChrom SB C18, 5 μm, 150 Å, 30×250 mm; 0.1% TFA in MeCN-0.10% TFA in water) affording 2-fluoro-3-(4-((1S,2S)-2-(4-fluorophenyl)cyclopropyl)-5′,6-dimethyl-2-oxo-2H-[1,4′-bipyridin]-2′-yl)-N,N-dimethylbenzamide (Example 12.1).

### Example 13.1 3-(3-bromo-4-((1S,2S)-2-(4-fluorophenyl)cyclopropyl)-5′,6-dimethyl-2-oxo-2H-[1,4′-bipyridin]-2′-yl)-2-fluoro-N,N-dimethylbenzamide

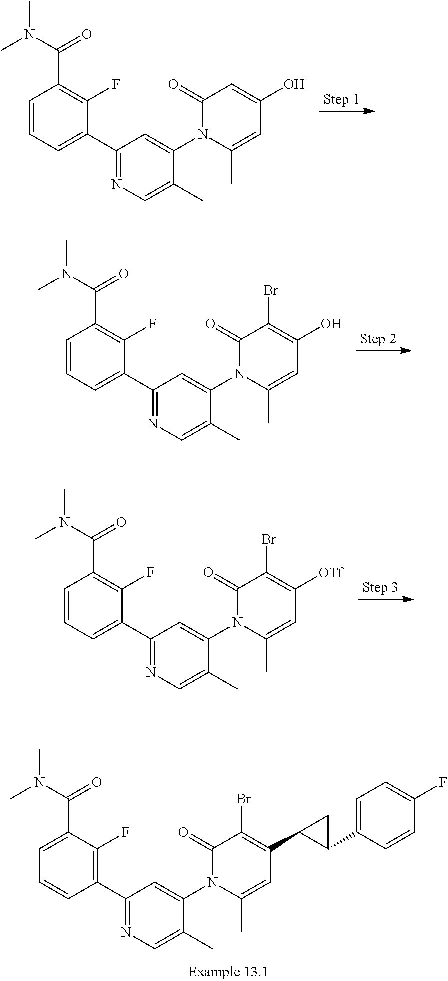 [SMILES: Cc1cnc(-c2cccc(C(=O)N(C)C)c2F)cc1-n1c(C)cc(O)cc1=O ; Cc1cnc(-c2cccc(C(=O)N(C)C)c2F)cc1-n1c(C)cc(O)c(Br)c1=O ; Cc1cnc(-c2cccc(C(=O)N(C)C)c2F)cc1-n1c(C)cc(OS(=O)(=O)C(F)(F)F)c(Br)c1=O ; Cc1cnc(-c2cccc(C(=O)N(C)C)c2F)cc1-n1c(C)cc([C@H]2C[C@@H]2c2ccc(F)cc2)c(Br)c1=O]

Step 1. 2-fluoro-3-(4-hydroxy-5′,6-dimethyl-2-oxo-2H-[1,4′-bipyridin]-2′-yl)-N,N-dimethylbenzamide (42 mg, 0.11 mmol) and sodium acetate (11 mg, 0.132 mmol) were suspended in acetic acid (3 mL) and cooled to −15° C. Bromine (0.0062 mL, 0.121 mmol) was added dropwise as a solution in acetic acid and the mixture was stirred for 15 minutes then transferred to a separatory funnel and diluted with water (10 mL). The mixture was then extracted 10% MeOH/DCM (3×10 mL). The combined organics were washed with sodium thiosulfate (sat. aq., 10 mL), then sodium bicarbonate (sat. aq., 10 mL). The organics were then concentrated and the residue was subjected to flash column chromatography (SiO₂, hexanes-EtOAc, then DCM-MeOH) to afford 3-(3-bromo-4-hydroxy-5′,6-dimethyl-2-oxo-2H-[1,4′-bipyridin]-2′-yl)-2-fluoro-N,N-dimethylbenzamide.

Step 2. This step was conducted analogously to Step 3 in the preparation of Example 12.1 affording 3-bromo-2′-(3-(dimethylcarbamoyl)-2-fluorophenyl)-5′,6-dimethyl-2-oxo-2H-[1,4′-bipyridin]-4-yl trifluoromethanesulfonate as a crude residue.

Step 3. This step was conducted analogously to Step 4 in the preparation of Example 12.1 affording 3-(3-bromo-4-((1S,2S)-2-(4-fluorophenyl)cyclopropyl)-5′,6-dimethyl-2-oxo-2H-[1,4′-bipyridin]-2′-yl)-2-fluoro-N,N-dimethylbenzamide (Example 13.1).

### Example 14.1: methyl (3-(3-chloro-4-((1S,2S)-2-(5-chloropyridin-3-yl)cyclopropyl)-5′,6-dimethyl-2-oxo-2H-[1,4′-bipyridin]-2′-yl)-2-fluorophenyl)carbamate

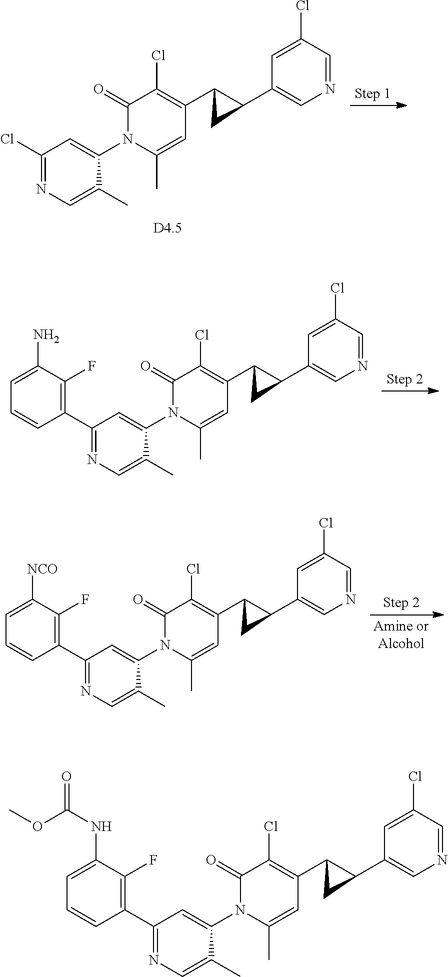

Step 1. 2′-(3-amino-2-fluorophenyl)-3-chloro-4-((1S,2S)-2-(5-chloropyridin-3-yl)cyclopropyl)-5′,6-dimethyl-2H-[1,4′-bipyridin]-2-one was prepared analogously to Example 6.2, using D4.5 instead of D4.1.

Step 2. A vial was charged with 2′-(3-amino-2-fluorophenyl)-3-chloro-4-((1S,2S)-2-(5-chloropyridin-3-yl)cyclopropyl)-5′,6-dimethyl-2H-[1,4′-bipyridin]-2-one (100 mg, 0.20 mmol), Et₃N (0.056 mL, 0.40 mmol), and DCM (1 mL). The mixture was cooled to 0° C., and triphosgene (60 mg, 0.20 mmol) was added. The mixture was allowed to warm to ambient temperature over 2 hrs., after which it was concentrated to provide 3-chloro-4-((1S,2S)-2-(5-chloropyridin-3-yl)cyclopropyl)-2′-(2-fluoro-3-isocyanatophenyl)-5′,6-dimethyl-2H-[1,4′-bipyridin]-2-one which was used without further purification.

Step 3. A vial was charged with 3-chloro-4-((1S,2S)-2-(5-chloropyridin-3-yl)cyclopropyl)-2′-(2-fluoro-3-isocyanatophenyl)-5′,6-dimethyl-2H-[1,4′-bipyridin]-2-one (20 mg, 0.038 mmol), Et₃N (0.032 mL, 0.23 mmol) and DCM (0.2 mL). Methanol (Alcohol) (0.1 mL) was added and the mixture was allowed to stir at ambient temperature for 18 hours, after which it was concentrated and subjected to reverse phase HPLC (Silicycle SiliaChrom SB C18, 5 μm, 150 Å, 30×250 mm; 0.1% TFA in MeCN-0.1% TFA in water) to yield methyl (3-(3-chloro-4-((1S,2S)-2-(5-chloropyridin-3-yl)cyclopropyl)-5′,6-dimethyl-2-oxo-2H-[1,4′-bipyridin]-2′-yl)-2-fluorophenyl)carbamate (Example 14.1).

The following compounds in Table 10 were prepared analogously to Example 14.1 modified as

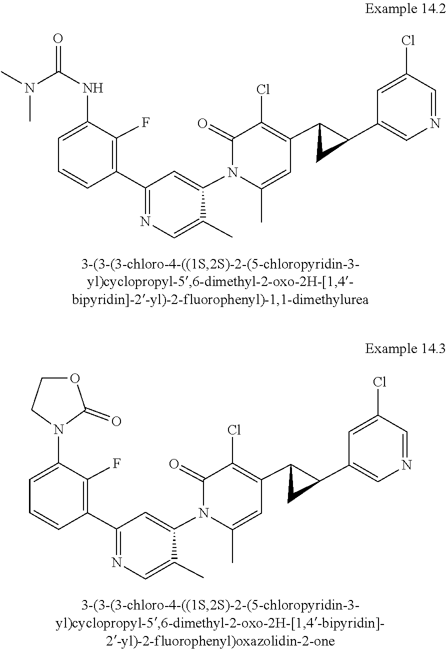

TABLE 10
Procedures analogous to Example 14.1 Synthesis

| Example | Starting | | Variation from |
|---|---|---|---|
| Number | Material | Amine or Alcohol | Example 14.1 |
| 14.2 | D4.5 | Dimethylamine | |
| 14.3 | D4.5 | 2-chloroethanol | The resulting product |
| | | | was dissolved in THF |
| | | | and treated with 2 |
| | | | equivalents of NaH |

### Example 15.1: N-(3-(3-chloro-4-((1S,2S)-2-(5-chloropyridin-3-yl)cyclopropyl)-5′,6-dimethyl-2-oxo-2H-[1,4′-bipyridin]-2′-yl)-2-fluorophenyl)-N,1-dimethylcyclopropane-1-carboxamide

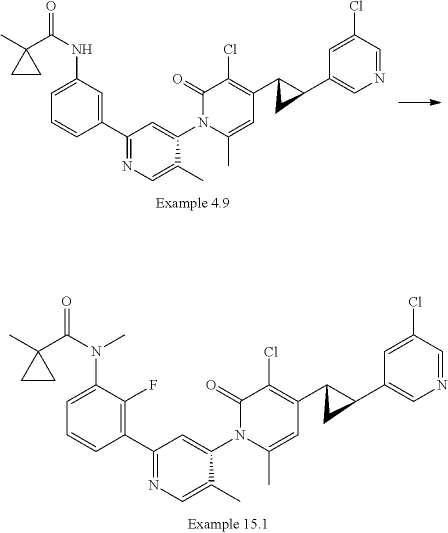

A vial was charged with N-(3-(3-chloro-4-((1S,2S)-2-(5-chloropyridin-3-yl)cyclopropyl)-5′,6-dimethyl-2-oxo-2H-[1,4′-bipyridin]-2′-yl)phenyl)-1-methylcyclopropane-1-carboxamide (13.0 mg, 0.023 mmol) and DMF (0.20 mL). NaH (60% dispersion in mineral oil, 1.8 mg, 0.045 mmol) was added followed by Mel (3.8 mg, 0.027 mmol). The mixture stirred for 18 hrs. NH₄Cl (conc. aq., 1 mL) was added and the mixture was extracted with EtOAc (3×1 mL). The combined organics were dried over Na₂SO₄, filtered, and concentrated. The residue was subjected to flash column chromatography (SiO₂, DCM-MeOH), followed by reverse phase HPLC (Silicycle SiliaChrom SB C18, 5 μm, 150 Å, 30×250 mm; 0.1% TFA in MeCN-0. 1% TFA in water) to yield N-(3-(3-chloro-4-((1S,2S)-2-(5-chloropyridin-3-yl)cyclopropyl)-5′,6-dimethyl-2-oxo-2H-[1,4′-bipyridin]-2′-yl)-2-fluorophenyl)-N,1-dimethylcyclopropane-1-carboxamide (Example 15.1).

### Example 16.1: 3-(3-chloro-4-((1S,2S)-2-(3-(cyclopropylcarbamoyl)-5-fluorophenyl)cyclopropyl)-5′,6-dimethyl-2-oxo-2H-[1,4′-bipyridin]-2′-yl)-2-fluoro-N,N-dimethylbenzamide

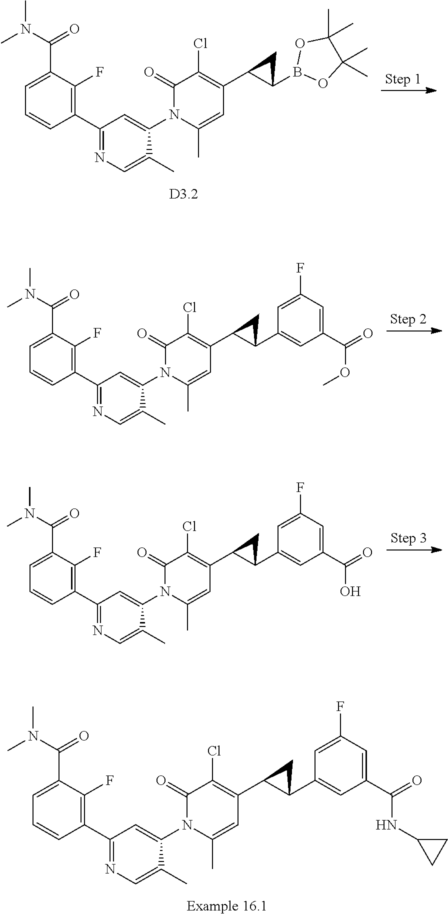

Step 1. Methyl 3-((1S,2S)-2-(3-chloro-2′-(3-(dimethylcarbamoyl)-2-fluorophenyl)-5′,6-dimethyl-2-oxo-2H-[1,4′-bipyridin]-4-yl)cyclopropyl)-5-fluorobenzoate was prepared following the procedure described in the preparation of Example 4.1, using D3.2 instead of D3.1, and methyl 3-bromo-5-fluorobenzoate in place of 3-bromo-5,6-dihydro-4H-pyrrolo[1,2-b]pyrazole. ES/MS: m/z 592.2 [M+H]⁺.

Step 2. To a solution of methyl 3-((1S,2S)-2-(3-chloro-2′-(3-(dimethylcarbamoyl)-2-fluorophenyl)-5′,6-dimethyl-2-oxo-2H-[1,4′-bipyridin]-4-yl)cyclopropyl)-5-fluorobenzoate (0.027 g, 0.045 mmol) in THF/MeOH (0.45 mL of a 5:1 mixture) was added LiOH (0.14 mL of a 1 M aq soln, 0.14 mmol). The mixture was stirred at 50° C. for 4 h. HCl (24 μL of a 6 M aq soln, 0.14 mmol) was added and the mixture was concentrated to yield 3-((1S,2S)-2-(3-chloro-2′-(3-(dimethylcarbamoyl)-2-fluorophenyl)-5′,6-dimethyl-2-oxo-2H-[1,4′-bipyridin]-4-yl)cyclopropyl)-5-fluorobenzoic acid, which was taken on without purification. ES/MS: m/z 578.1 [M+H]⁺.

Step 3. Example 16.1 was synthesized from 3-((1S,2S)-2-(3-chloro-2′-(3-(dimethylcarbamoyl)-2-fluorophenyl)-5′,6-dimethyl-2-oxo-2H-[1,4′-bipyridin]-4-yl)cyclopropyl)-5-fluorobenzoic acid following the procedure described in the preparation of Example 5.1, using cyclopropylamine instead of NH₄Cl.

### Example 17.1: 3-chloro-5-((1S,2S)-2-(3-chloro-3′-fluoro-2′-(2-fluoro-3-((R)-2-(trifluoromethyl)azetidine-1-carbonyl)phenyl)-5′,6-dimethyl-2-oxo-2H-[1,4′-bipyridin]-4-yl)cyclopropyl)pyridine 1-oxide

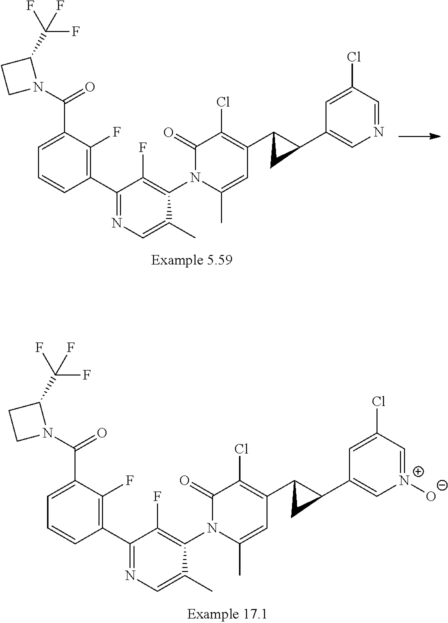

A vial was charged with 3-chloro-4-((1S,2S)-2-(5-chloropyridin-3-yl)cyclopropyl)-3′-fluoro-2′-(2-fluoro-3-((R)-2-(trifluoromethyl)azetidine-1-carbonyl)phenyl)-5′,6-dimethyl-2H-[1,4′-bipyridin]-2-one (Example 5.59, 10.0 mg, 0.015 mmol), 3-chloroperoxybenzoic acid (77% wt, 4.1 mg, 0.019 mmol), and DCM (1 mL). The mixture was heated to 40° C. for 30 min. The mixture was allowed to cool to ambient temperature, concentrated, and subjected to reverse phase HPLC (Silicycle SiliaChrom SB C18, 5 μm, 150 Å, 30×250 mm; 0.1% TFA in MeCN-0.1% TFA in water) to give 3-chloro-5-((1S,2S)-2-(3-chloro-3′-fluoro-2′-(2-fluoro-3-((R)-2-(trifluoromethyl)azetidine-1-carbonyl)phenyl)-5′,6-dimethyl-2-oxo-2H-[1,4′-bipyridin]-4-yl)cyclopropyl)pyridine 1-oxide (Example 17.1).

The following compounds in Table 11 were prepared analogously to Example 17.1 modified as described:

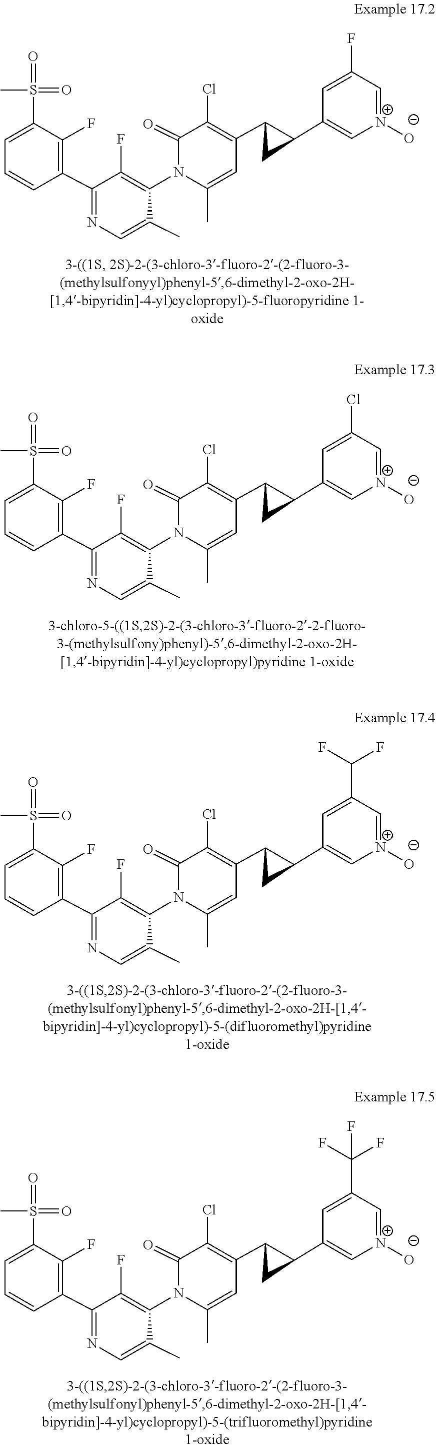

TABLE 11
Procedures analogous to Example 17.1 Synthesis

| Example | |
|---|---|
| Number | Starting Material |
| 17.2 | Example 6.33 |
| 17.3 | Example 6.36 |
| 17.4 | Example 4.62 |
| 17.5 | Example 4.63 |

Example 18.1: 5-chloro-3-(3-fluoro-2-(2-fluoro-3-(methylsulfonyl)phenyl)-5-methylpyridin-4-yl)-6-((1S,2S)-2-(5-fluoropyridin-3-yl)cyclopropyl)-2-methylpyrimidin-4(3H)-one. A vial was charged with D4.15 (50 mg, 0.12 mmol), C1.17 (51 mg, 0.17 mmol), Pd(dppf)Cl₂-DCM (9.7 mg, 0.012 mmol), Cs₂CO₃ (115 mg, 0.35 mmol), 1,4-dioxane (2.5 mL), and water (0.5 mL). The mixture was heated to 80° C. for 3 hrs, after which it was cooled to ambient temperature. Water (2 mL) was added, and the mixture was extracted with EtOAc (3×3 mL). The combine organics were dried over Na₂SO₄, filtered, and concentrated. The residue was subject to reverse phase HPLC (Silicycle SiliaChrom SB C18, 5 μm, 150 Å, 30×250 mm; 0.1% TFA in MeCN-0.1% TFA in water), to give 5-chloro-3-(3-fluoro-2-(2-fluoro-3-(methylsulfonyl)phenyl)-5-methylpyridin-4-yl)-6-((1S,2S)-2-(5-fluoropyridin-3-yl)cyclopropyl)-2-methylpyrimidin-4(3H)-one (Example 18.1).

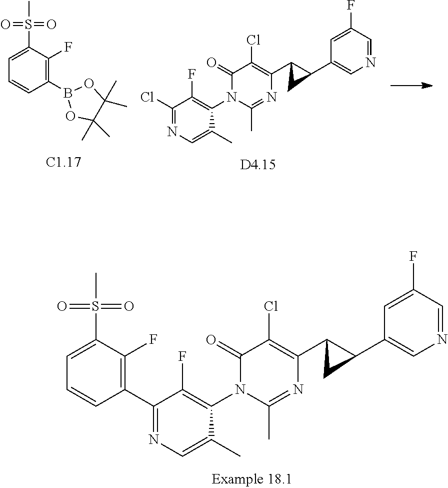

The following compounds in Table 12 were prepared analogously to Example 18.1 modified as described:

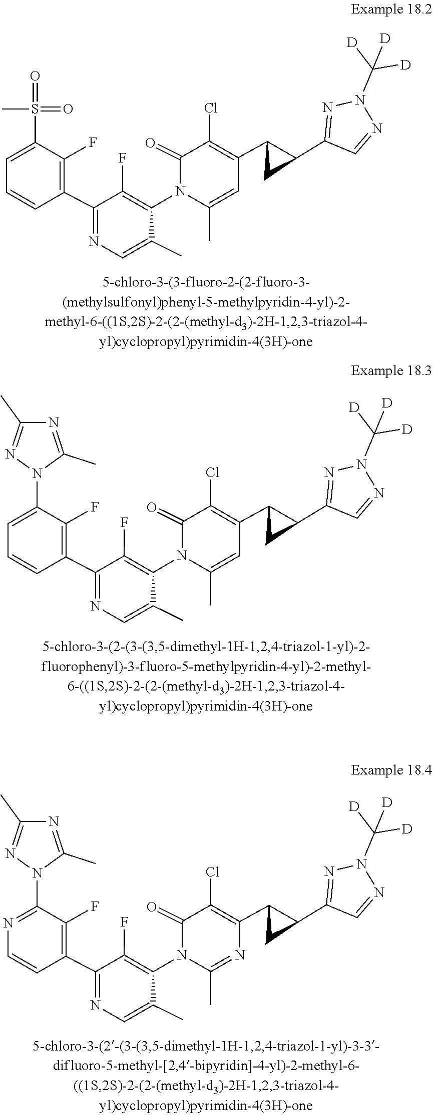

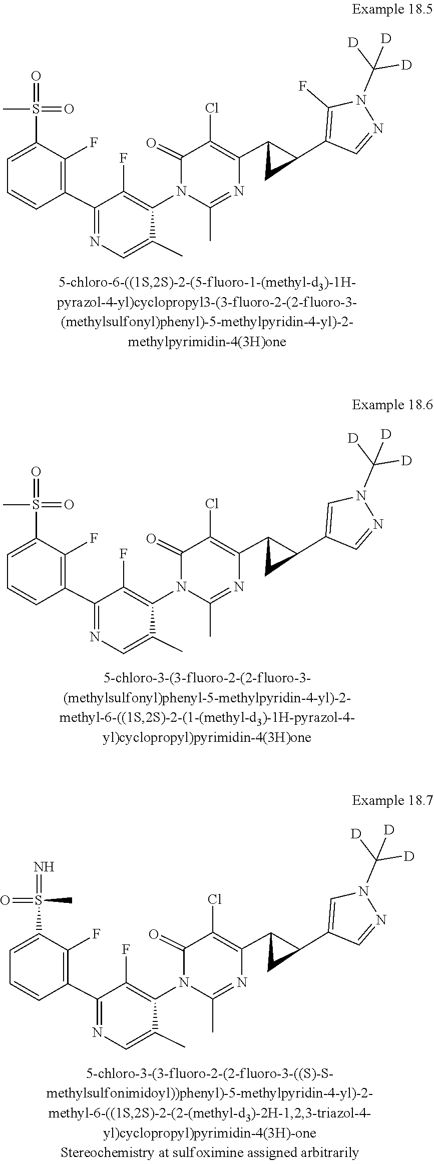

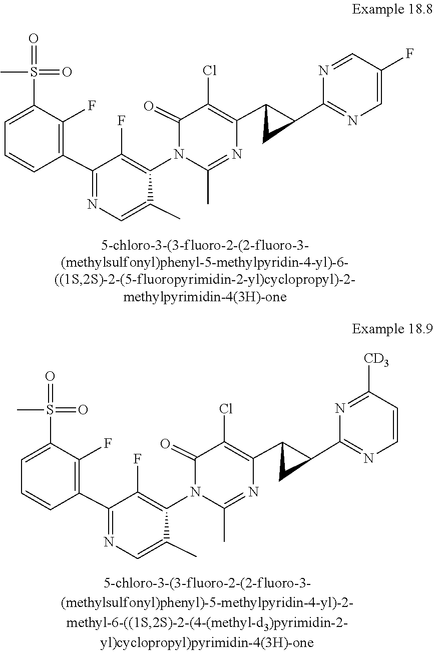

TABLE 12
Procedures analogous to Example 18.1 Synthesis

| Example Number | Variation from Example 18.1 |
|---|---|
| 18.2 | D4.14 instead of D4.15 |
| 18.3 | C13.3 instead of C1.17; D4.14 instead of D4.15 |
| 18.4 | C7.22 instead of C1.17; D4.14 instead of D4.15 |
| 18.5 | D4.17 instead of D4.15 |
| 18.6 | D4.16 instead of D4.15 |
| 18.7 | C10.1a instead of C1.17; D4.14 instead of D4.15; |
| | Boc-product was stirred in 1:1 TFA/DCM for 15 min |
| 18.8 | D4.19 instead of D4.15 |
| 18.9 | D4.18 instead of D4.15 |

Example 19.1: 5-chloro-3-(6′-(3,5-dimethyl-1H-1,2,4-triazol-1-yl)-3,5′-difluoro-5-methyl-[2,2′-bipyridin]-4-yl)-2-methyl-6-((1S,2S)-2-(2-(methyl-d3)-2H-1,2,3-triazol-4-yl)cyclopropyl)pyrimidin-4(3H)-one. A vial was charged with D4.14 (75 mg, 0.18 mmol), C16.1 (88 mg, 0.18 mmol), LiCl (31 mg, 0.73 mmol), CuI (10 mg, 0.055 mmol), Pd(PPh₃)₄ (21 mg, 0.018 mmol), and Me₂NAc (4 mL). The mixture was heated to 120° C. for 2 hr, was concentrated, and passed through a silica plug, washing with EtOAc. The mixture was concentrated and subject to reverse phase HPLC (Silicycle SiliaChrom SB C18, 5 μm, 150 Å, 30×250 mm; 0.1% TFA in MeCN-0.1% TFA in water) to give 5-chloro-3-(6′-(3,5-dimethyl-TH-1,2,4-triazol-1-yl)-3,5′-difluoro-5-methyl-[2,2′-bipyridin]-4-yl)-2-methyl-6-((1S,2S)-2-(2-(methyl-d3)-2H-1,2,3-triazol-4-yl)cyclopropyl)pyrimidin-4(3H)-one.

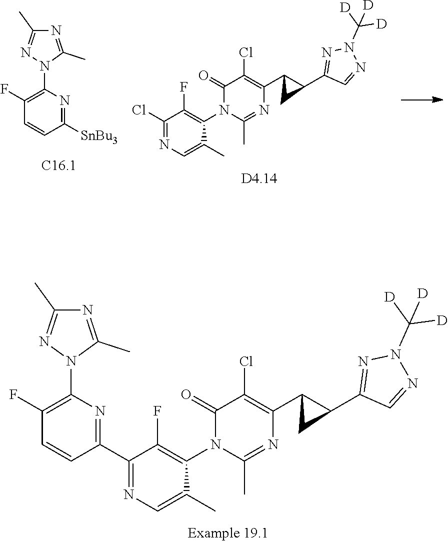

The following compounds in Table 13 were prepared analogously to Example 19.1 modified as described:

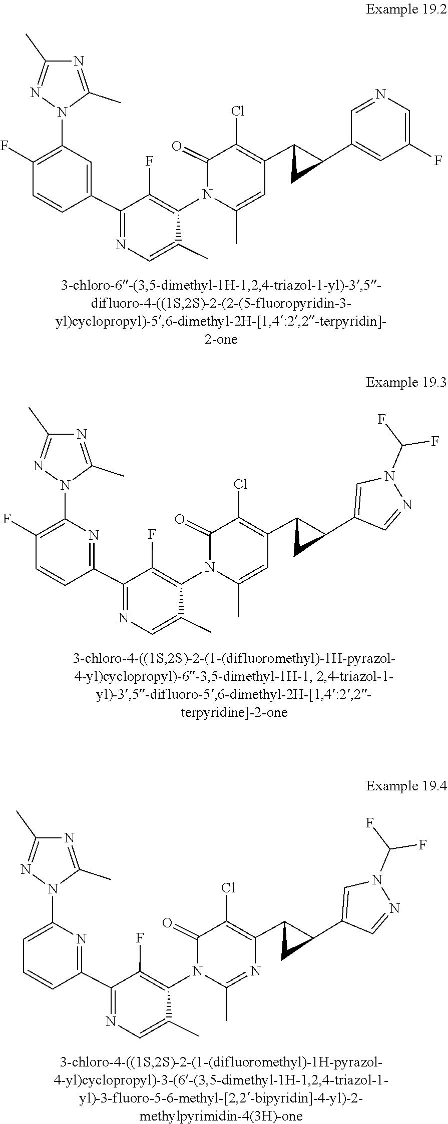

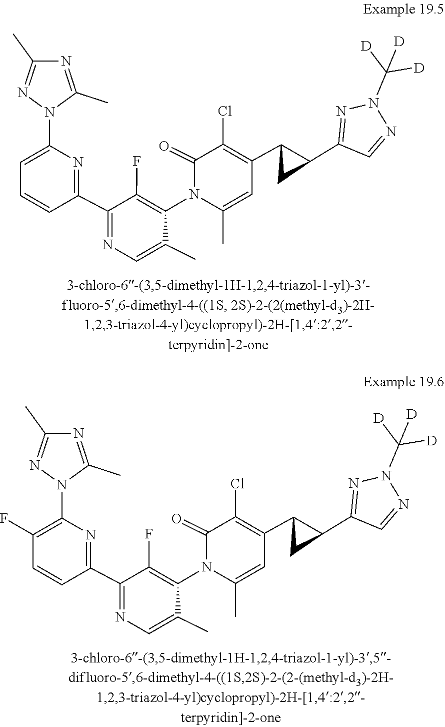

TABLE 13
Procedures analogous to Example 19.1 Synthesis

| Example Number | Variation from Example 19.1 |
|---|---|
| 19.2 | D4.10 instead of D4.14 |
| 19.3 | D4.11 isntead of D4.14 |
| 19.4 | C16.2 instead of C16.1; D4.11 instead of D4.14 |
| 19.5 | C16.2 instead of C16.1; D4.13 instead of D4.14 |
| 19.6 | D4.13 instead of D4.14 |

### Example 20.1: 3-chloro-4-((1S,2S)-2-(1-(difluoromethyl)-1H-pyrazol-4-yl)cyclopropyl)-2′-(3-((R)—N,S-dimethylsulfonimidoyl)-2-fluorophenyl)-3′-fluoro-5′,6-dimethyl-2H-[1,4′-bipyridin]-2-one

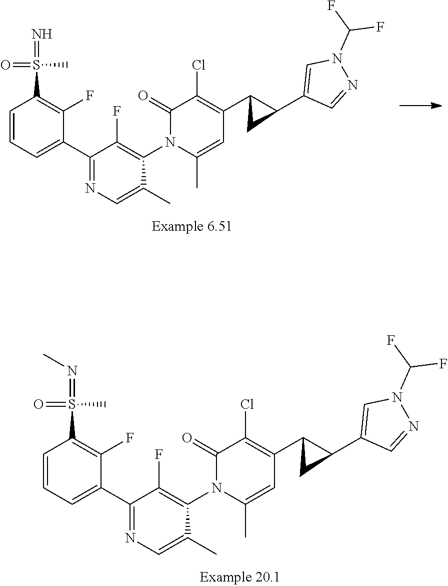

A vial was charged with 3-chloro-4-((1S,2S)-2-(1-(difluoromethyl)-1H-pyrazol-4-yl)cyclopropyl)-3′-fluoro-2′-(2-fluoro-3-((R)—S-methylsulfonimidoyl)phenyl)-5′,6-dimethyl-2H-[1,4′-bipyridin]-2-one (5.0 mg, 0.0074 mmol), Cu(OAc)₂ (2.0 mg, 0.011 mmol), pyridine (1.4 mg, 0.017 mmol), methylboronic acid (0.9 mg, 0.015 mmol), and 1,4-dioxane (1 mL). The mixture was heated to 100° C. for 2 h, cooled to room temperature, concentrated, and subject to reverse phase HPLC (Silicycle SiliaChrom SB C18, 5 μm, 150 Å, 30×250 mm; 0.1% TFA in MeCN-0.1% TFA in water) to give 3-chloro-4-((1S,2S)-2-(1-(difluoromethyl)-1H-pyrazol-4-yl)cyclopropyl)-2′-(3-((R)—N,S-dimethylsulfonimidoyl)-2-fluorophenyl)-3′-fluoro-5′,6-dimethyl-2H-[1,4′-bipyridin]-2-one.

The following compounds in Table 14 were prepared analogously to Example 20.1 modified as

| | |
|---|---|
| | Example 20.2 |
| 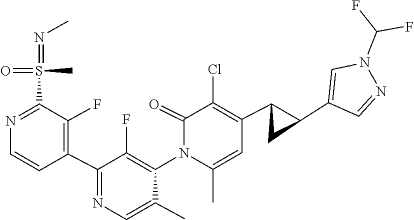 | |
| 3-chloro-4-((1S,2S)-2-(1-(difluoromethyl)-1H-pyrazol- | |
| 4-yl)cyclopropyl)-2″-((S)-N,S-dimethylsulfonimidoyl)- | |
| 3′,3″-difluoro-5′,6-dimethyl-2H-[1,4′:2′,4″-terpyridin]-2- | |
| one | |
| | Example 20.3 |
| 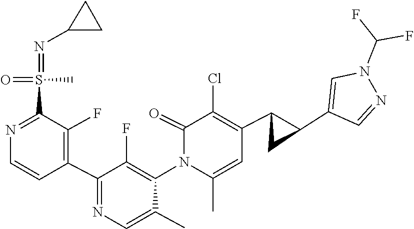 | |
| 3-chloro-2″-((R)-N-cyclopropyl-S- | |
| methylsulfonimidoyl)-4-((1S,2S)-2-(1-(difluoromethyl)- | |
| 1H-pyrazol-4-yl)cyclopropyl)-3′,3″-difluoro-5′,6- | |
| dimethyl-2H-[1,4′:2′,4″-terpyridin]-2-one | |
| | Example 20.4 |
| 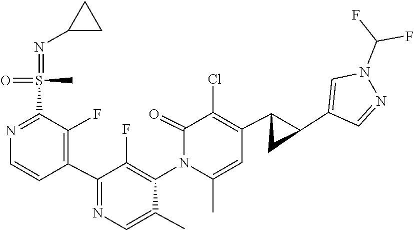 | |
| 3-chloro-2″-((S)-N-cyclopropyl-S- | |
| methylsulfonimidoyl)-4-((1S,2S)-2-(1-(difluoromethyl)- | |
| 1H-pyrazol-4-yl)cyclopropyl)-3′,3″-difluoro-5′,6- | |
| dimethyl-2H-[1,4′:2′,4″-terpyridin]-2-one | |

| | Starting | |
|---|---|---|
| Example Number | Material | Boronic acid |
| 20.2 | Example 6.51 | methylboronic acid |
| 20.3 | Example 6.52 | cyclopropylboronic acid |
| 20.4 | Example 6.52 | cyclopropylboronic acid |

### Example 21.1: N-(2-(4-(3-chloro-3′-fluoro-4-((1S,2S)-2-(5-fluoropyridin-3-yl)cyclopropyl)-5′,6-dimethyl-2-oxo-2H-[1,4′-bipyridin]-2′-yl)pyrimidin-2-yl)propan-2-yl)acetamide

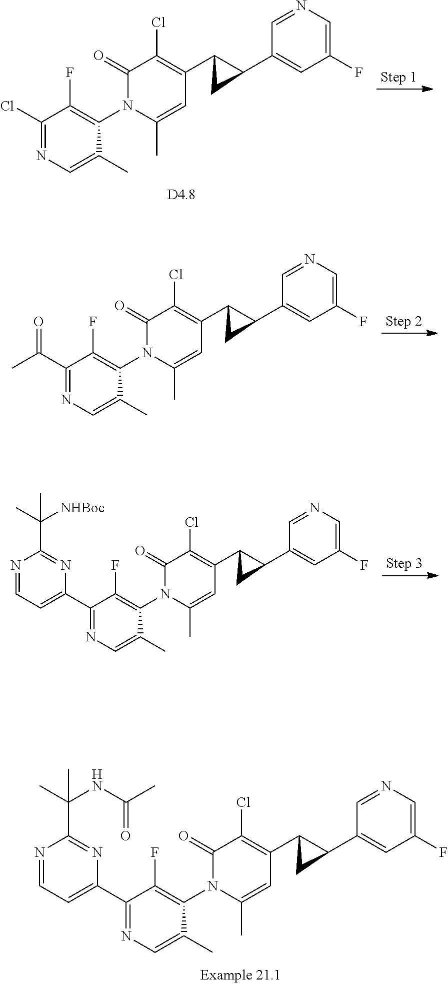

Step 1. A vial was charged with D4.8 (160 mg, 0.38 mmol), tributyl(1-ethoxyvinyl)stannane (301 mg, 0.83 mmol), Pd(dppf)Cl₂-DCM (31 mg, 0.038 mmol), CuI (7.2 mg, 0.038 mmol), and 1,4-dioxane (5 mL). The mixture was heated to 95° C. for 3 hrs. The mixture was cooled to ambient temperature, and THF (3 mL), water (1 mL), and HCl (6M, 0.2 mL) was added. The mixture stirred for 2 hrs at ambient temperature. Water (5 mL) was added, and the mixture was extracted with EtOAc (3×5 mL). The combined organic layers were dried over Na₂SO₄, filtered, and concentrated. The residue was subject to flash column chromatography (SiO₂, 10-70% acetone in hexanes) to afford (2′-acetyl-3-chloro-3′-fluoro-4-((1S,2S)-2-(5-fluoropyridin-3-yl)cyclopropyl)-5′,6-dimethyl-2H-[1,4′-bipyridin]-2-one. ES/MS m/z: 430.1

Step 2. A vial was charged with (2′-acetyl-3-chloro-3′-fluoro-4-((1S,2S)-2-(5-fluoropyridin-3-yl)cyclopropyl)-5′,6-dimethyl-2H-[1,4′-bipyridin]-2-one (97 mg, 0.23 mmol) and DMF (1.5 mL). N,N-dimethylforamide dimethyl acetal (108 mg, 0.90 mmol) was added and the mixture was heated to 70° C. for 3 hrs. The mixture was concentrated under vacuum. The resulting residue was dissolved in DMF (1.5 mL), and K₂CO₃ (155 mg, 1.12 mmol), tert-butyl (1-amino-1-imino-2-methylpropan-2-yl)carbamate, acetic acid salt (188 mg, 0.72 mmol) were added. The mixture was heated to 70° C. for 3 hrs. The mixture was cooled to ambient temperature, and water (5 mL) was added. The resulting precipitate was filtered, washed with water, and dried under vacuum to afford tert-butyl (2-(4-(3-chloro-3′-fluoro-4-((1S,2S)-2-(5-fluoropyridin-3-yl)cyclopropyl)-5′,6-dimethyl-2-oxo-2H-[1,4′-bipyridin]-2′-yl)pyrimidin-2-yl)propan-2-yl)carbamate. ES/MS m/z: 622.8

Step 3. A vial was charged with tert-butyl (2-(4-(3-chloro-3′-fluoro-4-((1S,2S)-2-(5-fluoropyridin-3-yl)cyclopropyl)-5′,6-dimethyl-2-oxo-2H-[1,4′-bipyridin]-2′-yl)pyrimidin-2-yl)propan-2-yl)carbamate (66 mg, 0.11 mmol), DCM (3.5 mL), followed by HCl (6M in dioxane, 0.19 mL, 0.74 mmol). The mixture stirred at ambient temperature for 18 hr., and was then concentrated under vacuum. The residue was dissolved in DCM (3.5 mL), and Et₃N (0.18 ml, 1.3 mmol) was added followed by acetic anhydride (0.040 mL, 0.42 mmol). The mixture was stirred for 1 hr at ambient temperature and then concentrated. The residue was subject to reverse phase HPLC (Silicycle SiliaChrom SB C18, 5 μm, 150 Å, 30×250 mm 0.1% TFA in MeCN-0.1% TFA in water) to give N-(2-(4-(3-chloro-3′-fluoro-4-((1 S,2S)-2-(5-fluoropyridin-3-yl)cyclopropyl)-5′,6-dimethyl-2-oxo-2H-[1,4′-bipyridin]-2′-yl)pyrimidin-2-yl)propan-2-yl)acetamide.

### Example 22.1: 3-chloro-3′-fluoro-4-((1S,2S)-2-(5-fluoropyridin-3-yl)cyclopropyl)-2′-(3-(2-hydroxypropan-2-yl)-1H-pyrazol-1-yl)-5′,6-dimethyl-2H-[1,4′-bipyridin]-2-one

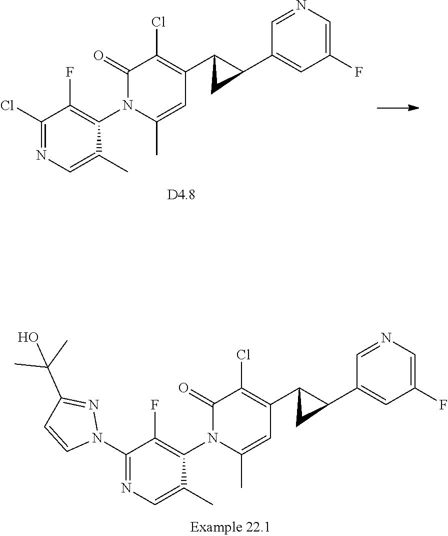

To a stirred mixture of D4.8 (60 mg, 0.142 mmol), 2-(1H-pyrazol-3-yl)propan-2-ol (54 mg, 0.426 mmol) in 1,4-dioxane (0.7 mL) were added N′,N-dimethylcyclohexane-1,2-diamine (40 mg, 0.284 mmol), Cu (27 mg, 0.142-mmol), K₂C₃ (39 mg, 0.284 mmol) and NaI (43 mg, 0.284 mmol) at room temperature under nitrogen atmosphere. The resulting mixture was stirred overnight at 100° C. under nitrogen atmosphere. The mixture was allowed to cool don to room temperature. The resulting mixture was diluted with EtOAc (3 mL), then washed with water (2×3 mL), and NaCl (sat. aq., 1×3 mL). The organic layer was dried over Na₂SO₄, filtered and concentrated. The residue was purified by silica gel column chromatography (0-100% EtOAc in hex, then 0-40′ MOH in EtOAc). The residue was further subject to reverse phase HPLC (Silicycle SiliaChrom SB C18, 5 am, 150 Å, 30×250 mm; 0.1% TFA in MeCN-0.1003TFA in water) to give 3-chloro-3′-fluoro-4-((1S,2S)-2-(5-fluoropyridin-3-yl)cyclopropyl)-2′-(3-(2-hydroxypropan-2-yl)-1H-pyrazol-1-yl)-5′,6-dimethyl-2H-[1,4′-bipyridin]-2-one. IUPAC names of compounds prepared in the Final Procedures are provided in Table 13.

TABLE 15
IUPAC names of compounds prepared in Final Procedures

| Example | |
|---|---|
| Number | IUPAC Name |
| 1.1 | 4-((1S,2R)-2-benzylcyclopropyl)-3-chloro-2′-(2-(2-hydroxypropan-2-yl)pyrimidin- |
| | 4-yl)-5′,6-dimethyl-2H-[1,4′-bipyridin]-2-one |
| 1.2 | 3-chloro-2′-(2-(2-hydroxypropan-2-yl)pyrimidin-4-yl)-4-((1S,2S)-2-(3- |
| | methoxyphenyl)cyclopropyl)-5′,6-dimethyl-2H-[1,4′-bipyridin]-2-one |
| 1.3 | 3-chloro-4-((1S,2S)-2-(4-fluorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.4 | 3-chloro-4-((1S,2S)-2-(4-fluorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.5 | 3-chloro-4-((1R,2R)-2-(4-fluorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.6 | 3-chloro-4-((1S,2S)-2-(2,4-difluorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.7 | 3-chloro-4-((1S,2S)-2-(3,4-difluorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.8 | 3-chloro-4-((1S,2S)-2-(2,2-difluorobenzo[d][1,3]dioxol-5-yl)cyclopropyl)-2′-(2-(2- |
| | hydroxypropan-2-yl)pyrimidin-4-yl)-5′,6-dimethyl-2H-[1,4′-bipyridin]-2-one |
| 1.9 | 3-chloro-4-((1S,2S)-2-(4-chloro-3-fluorophenyl)cyclopropyl)-2′-(2-(2- |
| | hydroxypropan-2-yl)pyrimidin-4-yl)-5′,6-dimethyl-2H-[1,4′-bipyridin]-2-one |
| 1.10 | 3-chloro-4-((1S,2S)-2-(2,6-difluorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.11 | 3-chloro-4-((1S,2S)-2-(3-chloro-4-fluorophenyl)cyclopropyl)-2′-(2-(2- |
| | hydroxypropan-2-yl)pyrimidin-4-yl)-5′,6-dimethyl-2H-[1,4′-bipyridin]-2-one |
| 1.12 | 3-chloro-4-((1S,2S)-2-(2-chlorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.13 | 3-chloro-4-((1S,2S)-2-(4-cyclopropylphenyl)cyclopropyl)-2′-(2-(2-hydroxypropan- |
| | 2-yl)pyrimidin-4-yl)-5′,6-dimethyl-2H-[1,4′-bipyridin]-2-one |
| 1.14 | 3-chloro-4-((1S,2S)-2-(5-(difluoromethoxy)pyridin-2-yl)cyclopropyl)-2′-(2-(2- |
| | hydroxypropan-2-yl)pyrimidin-4-yl)-5′,6-dimethyl-2H-[1,4′-bipyridin]-2-one |
| 1.15 | 3-chloro-2′-(2-(2-hydroxypropan-2-yl)pyrimidin-4-yl)-5′,6-dimethyl-4-((1S,2S)-2- |
| | (2,3,4-trifluorophenyl)cyclopropyl)-2H-[1,4′-bipyridin]-2-one |
| 1.16 | 3-chloro-4-((1R,2R)-2-(1-(difluoromethyl)-1H-pyrazol-4-yl)cyclopropyl)-2′-(2-(2- |
| | hydroxypropan-2-yl)pyrimidin-4-yl)-5′,6-dimethyl-2H-[1,4′-bipyridin]-2-one |
| 1.17 | 3-chloro-4-((1S,2S)-2-(1-(difluoromethyl)-1H-pyrazol-4-yl)cyclopropyl)-2′-(2-(2- |
| | hydroxypropan-2-yl)pyrimidin-4-yl)-5′,6-dimethyl-2H-[1,4′-bipyridin]-2-one |
| 1.18 | 3-chloro-4-((1S,2S)-2-(6-cyclopropylpyridin-2-yl)cyclopropyl)-2′-(2-(2- |
| | hydroxypropan-2-yl)pyrimidin-4-yl)-5′,6-dimethyl-2H-[1,4′-bipyridin]-2-one |
| 1.19 | 3-chloro-2′-(2-(2-hydroxypropan-2-yl)pyrimidin-4-yl)-5′,6-dimethyl-4-((1S,2S)-2- |
| | (2-methylthiazol-4-yl)cyclopropyl)-2H-[1,4′-bipyridin]-2-one |
| 1.20 | 4-((1S,2S)-2-(3-chloro-2′-(2-(2-hydroxypropan-2-yl)pyrimidin-4-yl)-5′,6-dimethyl- |
| | 2-oxo-2H-[1,4′-bipyridin]-4-yl)cyclopropyl)-2-fluorobenzonitrile |
| 1.21 | 4-((1S,2S)-2-(3-chloro-2′-(2-(2-hydroxypropan-2-yl)pyrimidin-4-yl)-5′,6-dimethyl- |
| | 2-oxo-2H-[1,4′-bipyridin]-4-yl)cyclopropyl)benzonitrile |
| 1.22 | 3-chloro-2′-(2-(2-hydroxypropan-2-yl)pyrimidin-4-yl)-5′,6-dimethyl-4-((1S,2S)-2- |
| | (5-(trifluoromethyl)pyridin-2-yl)cyclopropyl)-2H-[1,4′-bipyridin]-2-one |
| 1.23 | 3-chloro-2′-(2-(2-hydroxypropan-2-yl)pyrimidin-4-yl)-5′,6-dimethy1-4-((1S,2S)-2- |
| | (6-(trifluoromethyl)pyridin-3-yl)cyclopropyl)-2H-[1,4′-bipyridin]-2-one |
| 1.24 | 3-chloro-2′-(2-(2-hydroxypropan-2-yl)pyrimidin-4-yl)-5′,6-dimethyl-4-((1S,2S)-2- |
| | (5-(trifluoromethoxy)pyridin-2-yl)cyclopropyl)-2H-[1,4′-bipyridin]-2-one |
| 1.25 | 3-chloro-2′-(2-(2-hydroxypropan-2-yl)pyrimidin-4-yl)-5′,6-dimethyl-4-((1S,2S)-2- |
| | (6-(trifluoromethoxy)pyridin-3-yl)cyclopropyl)-2H-[1,4′-bipyridin]-2-one |
| 1.26 | 3-chloro-2′-(2-(2-hydroxypropan-2-yl)pyrimidin-4-yl)-5′,6-dimethy1-4-((1R,2R)-2- |
| | (2-(trifluoromethyl)thiazol-5-yl)cyclopropyl)-2H-[1,4′-bipyridin]-2-one |
| 1.27 | 3-chloro-2′-(2-(2-hydroxypropan-2-yl)pyrimidin-4-yl)-5′,6-dimethy1-4-((1S,2S)-2- |
| | (2-(trifluoromethyl)thiazol-5-yl)cyclopropyl)-2H-[1,4′-bipyridin]-2-one |
| 1.28 | 3-chloro-4-((1S,2S)-2-(4-chloro-2-fluorophenyl)cyclopropyl)-2′-(2-(2- |
| | hydroxypropan-2-yl)pyrimidin-4-yl)-5′,6-dimethyl-2H-[1,4′-bipyridin]-2-one |
| 1.29 | 3-chloro-2′-(2-(2-hydroxypropan-2-yl)pyrimidin-4-yl)-5′,6-dimethy1-4-((1S,2S)-2- |
| | (3-(trifluoromethoxy)phenyl)cyclopropyl)-2H-[1,4′-bipyridin]-2-one |
| 1.30 | 3-chloro-2′-(2-(2-hydroxypropan-2-yl)pyrimidin-4-yl)-5′,6-dimethyl-4-((1S,2S)-2- |
| | (4-(trifluoromethyl)phenyl)cyclopropyl)-2H-[1,4′-bipyridin]-2-one |
| 1.31 | 4-((1S,2S)-2-(3-chloro-2′-(2-(2-hydroxypropan-2-yl)pyrimidin-4-yl)-5′,6-dimethyl- |
| | 2-oxo-2H-[1,4′-bipyridin]-4-yl)cyclopropyl)picolinonitrile |
| 1.32 | 5-((1S,2S)-2-(3-chloro-2′-(2-(2-hydroxypropan-2-yl)pyrimidin-4-yl)-5′,6-dimethyl- |
| | 2-oxo-2H-[1,4′-bipyridin]-4-yl)cyclopropyl)nicotinonitrile |
| 1.33 | 3-chloro-2′-(2-(2-hydroxypropan-2-yl)pyrimidin-4-yl)-5′,6-dimethy1-4-((1R,2R)-2- |
| | (5-(trifluoromethyl)pyridin-3-yl)cyclopropyl)-2H-[1,4′-bipyridin]-2-one |
| 1.34 | 3-chloro-2′-(2-(2-hydroxypropan-2-yl)pyrimidin-4-yl)-5′,6-dimethyl-4-((1S,2S)-2- |
| | (5-(trifluoromethyl)pyridin-3-yl)cyclopropyl)-2H-[1,4′-bipyridin]-2-one |
| 1.35 | methyl 3-((1S,2S)-2-(3-chloro-2′-(2-(2-hydroxypropan-2-yl)pyrimidin-4-yl)-5′,6- |
| | dimethy1-2-oxo-2H-[1,4′-bipyridin]-4-yl)cyclopropyl)benzoate |
| 1.36 | 3-chloro-4-((1S,3S)-2,2-difluoro-3-phenylcyclopropyl)-2′-(2-(2-hydroxypropan-2- |
| | yl)pyrimidin-4-yl)-5′,6-dimethyl-2H-[1,4′-bipyridin]-2-one |
| 1.37 | 3-chloro-2′-(2-(2-hydroxypropan-2-yl)pyrimidin-4-yl)-5′,6-dimethyl-4-((1S,2S)-2- |
| | (2,3,6-trifluorophenyl)cyclopropyl)-2H-[1,4′-bipyridin]-2-one |
| 1.38 | 3-chloro-4-((1S,2S)-2-(4-fluoro-3-(trifluoromethyl)phenyl)cyclopropyl)-2′-(2-(2- |
| | hydroxypropan-2-yl)pyrimidin-4-yl)-5′,6-dimethyl-2H-[1,4′-bipyridin]-2-one |
| 1.39 | 3-chloro-2′-(2-(2-hydroxypropan-2-yl)pyrimidin-4-yl)-5′,6-dimethy1-4-((1S,2S)-2- |
| | phenylcyclopropyl)-2H-[1,4′-bipyridin]-2-one |
| 1.40 | 3-chloro-2′-(2-(2-hydroxypropan-2-yl)pyrimidin-4-yl)-5′,6-dimethyl-4-((1S,2S)-2- |
| | (2-(trifluoromethyl)pyrimidin-4-yl)cyclopropyl)-2H-[1,4′-bipyridin]-2-one |
| 1.41 | 3-chloro-2′-(2-(2-hydroxypropan-2-yl)pyrimidin-4-yl)-5′,6-dimethy1-4-((1R,2R)-2- |
| | (2-(trifluoromethyl)pyrimidin-4-yl)cyclopropyl)-2H-[1,4′-bipyridin]-2-one |
| 1.42 | 3-chloro-2′-(2-(2-hydroxypropan-2-yl)pyrimidin-4-yl)-5′,6-dimethyl-4-((1S,2S)-2- |
| | (pyridin-2-yl)cyclopropyl)-2H-[1,4′-bipyridin]-2-one |
| 1.43 | 3-chloro-4-((1S,2S)-2-(1-(difluoromethyl)-1H-pyrazol-3-yl)cyclopropyl)-2′-(2-(2- |
| | hydroxypropan-2-yl)pyrimidin-4-yl)-5′,6-dimethyl-2H-[1,4′-bipyridin]-2-one |
| 1.44 | 3-chloro-4-((1S,2R)-2-(4,4-difluorocyclohexyl)cyclopropyl)-2′-(2-(2- |
| | hydroxypropan-2-yl)pyrimidin-4-yl)-5′,6-dimethyl-2H-[1,4′-bipyridin]-2-one |
| 1.45 | 3-chloro-4-((1S,2S)-2-(1-(difluoromethyl)-1H-pyrazol-4-yl)cyclopropyl)-2′-(2-(2- |
| | hydroxypropan-2-yl)pyrimidin-4-yl)-5′,6-dimethy1-2H-[1,4′-bipyridin]-2-one |
| 1.46 | 3-chloro-2′-(2-(2-hydroxypropan-2-yl)pyrimidin-4-yl)-4-((1S,2S)-2-(6- |
| | methoxypyridin-2-yl)cyclopropyl)-5′,6-dimethyl-2H-[1,4′-bipyridin]-2-one |
| 1.47 | 3-((1S,2S)-2-(3-chloro-2′-(2-(2-hydroxypropan-2-yl)pyrimidin-4-yl)-5′,6-dimethyl- |
| | 2-oxo-2H-[1,4′-bipyridin]-4-yl)cyclopropyl)-4-fluorobenzonitrile |
| 1.48 | 3-chloro-4-(2-(2-fluorophenyl)cyclopropyl)-2′-(2-(2-hydroxypropan-2- |
| | yl)pyrimidin-4-yl)-5′,6-dimethy1-2H-[1,4′-bipyridin]-2-one |
| 1.49 | 3-chloro-4-(2-(3-fluorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.50 | 3-(2-(3-chloro-2′-(2-(2-hydroxypropan-2-yl)pyrimidin-4-yl)-5′,6-dimethyl-2-oxo- |
| | 2H-[1,4′-bipyridin]-4-yl)cyclopropyl)benzonitrile |
| 1.51 | 3-(2-(3-chloro-2′-(2-(2-hydroxypropan-2-yl)pyrimidin-4-yl)-5′,6-dimethyl-2-oxo- |
| | 2H-[1,4′-bipyridin]-4-yl)cyclopropyl)-5-fluorobenzonitrile |
| 1.52 | 3-chloro-4-((1S,2S)-2-(3-fluoropyridin-4-yl)cyclopropyl)-2′-(2-(2-hydroxypropan- |
| | 2-yl)pyrimidin-4-yl)-5′,6-dimethyl-2H-[1,4′-bipyridin]-2-one |
| 1.53 | 3-chloro-4-((1S,2S)-2-(5-fluoropyridin-3-yl)cyclopropyl)-2′-(2-(2-hydroxypropan- |
| | 2-yl)pyrimidin-4-yl)-5′,6-dimethyl-2H-[1,4′-bipyridin]-2-one |
| 1.54 | 3-chloro-4-(2′,3′-dihydrospiro[cyclopropane-1,l′-inden]-2-yl)-2′-(2-(2- |
| | hydroxypropan-2-yl)pyrimidin-4-yl)-5′,6-dimethyl-2H-[1,4′-bipyridin]-2-one |
| 1.55 | 3-chloro-4-((1S,2S)-2-(3,4-difluorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.56 | 3-chloro-2′-(2-(2-hydroxypropan-2-yl)pyrimidin-4-yl)-5′,6-dimethy1-4-((1S,2S)-2- |
| | (3,4,5-trifluorophenyl)cyclopropyl)-2H-[1,4′-bipyridin]-2-one |
| 1.57 | 3-chloro-2′-(2-(2-hydroxypropan-2-yl)pyrimidin-4-yl)-5′,6-dimethyl-4-((1S,2S)-2- |
| | (2,4,5-trifluorophenyl)cyclopropyl)-2H-[1,4′-bipyridin]-2-one |
| 1.58 | 3-chloro-4-((1S,2S)-2-(3,5-difluorophenyl)cyclopropyl)-2′-(2-(2-hydroxypropan-2- |
| | yl)pyrimidin-4-yl)-5′,6-dimethyl-2H-[1,4′-bipyridin]-2-one |
| 1.59 | 3-chloro-2′-(2-(2-hydroxypropan-2-yl)pyrimidin-4-yl)-5′,6-dimethyl-4-((1S,2S)-2- |
| | (3-(trifluoromethyl)phenyl)cyclopropyl)-2H-[1,4′-bipyridin]-2-one |
| 1.60 | 3-chloro-2′-(2-(2-hydroxypropan-2-yl)pyrimidin-4-yl)-5′,6-dimethy1-4-((1S,2S)-2- |
| | (1-(trifluoromethyl)-1H-pyrazol-4-yl)cyclopropyl)-2H-[1,4′-bipyridin]-2-one |
| 1.61 | 3-chloro-2′-(2-(2-hydroxypropan-2-yl)pyrimidin-4-yl)-5′,6-dimethy1-4-((1R,2R)-2- |
| | (1-(trifluoromethyl)-1H-pyrazol-4-yl)cyclopropyl)-2H-[1,4′-bipyridin]-2-one |
| 1.62 | 3-chloro-2′-(2-(2-hydroxypropan-2-yl)pyrimidin-4-yl)-5′,6-dimethyl-4-((1S,2S)-2- |
| | (2,4,6-trifluorophenyl)cyclopropyl)-2H-[1,4′-bipyridin]-2-one |
| 1.63 | 3-chloro-2′-(2-(2-hydroxypropan-2-yl)pyrimidin-4-yl)-5′,6-dimethyl-4-((1S,2S)-2- |
| | (5-(trifluoromethyl)pyrazin-2-yl)cyclopropyl)-2H-[1,4′-bipyridin]-2-one |
| 1.64 | 3-chloro-3′-fluoro-4-((1S,2S)-2-(4-fluorophenyl)cyclopropyl)-2′-(2-(2- |
| | hydroxypropan-2-yl)pyrimidin-4-yl)-5′,6-dimethyl-2H-[1,4′-bipyridin]-2-one |
| 1.65 | 3-chloro-3′-fluoro-2′-(2-(2-hydroxypropan-2-yl)pyrimidin-4-yl)-5′,6-dimethyl-4- |
| | ((1S,2S)-2-(2-(methyl-d3)-2H-1,2,3-triazol-4-yl)cyclopropyl)-2H-[1,4′-bipyridin]-2- |
| | one |
| 2.1 | 4-((1S,2S)-2-(3-amino-4-fluorophenyl)cyclopropyl)-3-chloro-2′-(2-(2- |
| | hydroxypropan-2-yl)pyrimidin-4-yl)-5′,6-dimethyl-2H-[1,4′-bipyridin]-2-one |
| 2.2 | N-(5-((1S,2S)-2-(3-chloro-2′-(2-(2-hydroxypropan-2-yl)pyrimidin-4-yl)-5′,6- |
| | dimethyl-2-oxo-2H-[1,4′-bipyridin]-4-yl)cyclopropyl)-2-fluorophenyl)acetamide |
| 2.3 | N-(5-((1S,2S)-2-(3-chloro-2′-(2-(2-hydroxypropan-2-yl)pyrimidin-4-yl)-5′,6- |
| | dimethyl-2-oxo-2H-[1,4′-bipyridin]-4-yl)cyclopropyl)-2- |
| | fluorophenyl)cyclopropanecarboxamide |
| 3.1 | 3-(3-chloro-4-((1S,2S)-2-(4-fluorophenyl)cyclopropyl)-5′,6-dimethyl-2-oxo-2H- |
| | [1,4′-bipyridin]-2′-yl)-2-fluoro-N,N-dimethylbenzamide |
| 3.2 | 3-(3-chloro-4-(2-(3,4-difluorophenyl)-2-methylcyclopropyl)-5′,6-dimethyl-2-oxo- |
| | 2H-[1,4′-bipyridin]-2′-yl)-2-fluoro-N,N-dimethylbenzamide |
| 3.3 | 3-(3-chloro-4-((1S,2S)-2-(5-chloropyridin-3-yl)cyclopropyl)-5′,6-dimethyl-2-oxo- |
| | 2H-[1,4′-bipyridin]-2′-yl)-2-fluoro-N,N-dimethylbenzamide |
| 3.4 | 3-(3-chloro-5′,6-dimethyl-2-oxo-4-((1S,2S)-2-(o-tolyl)cyclopropyl)-2H-[1,4′- |
| | bipyridin]-2′-yl)-2-fluoro-N,N-dimethylbenzamide |
| 3.5 | 3-(3-chloro-4-((1S,2S)-2-(2,6-difluorophenyl)cyclopropyl)-5′,6-dimethyl-2-oxo-2H- |
| | [1,4′-bipyridin]-2′-yl)-2-fluoro-N,N-dimethylbenzamide |
| 3.6 | 3-(3-chloro-4-((1S,2S)-2-(4-chlorophenyl)cyclopropyl)-5′,6-dimethyl-2-oxo-2H- |
| | [1,4′-bipyridin]-2′-yl)-2-fluoro-N,N-dimethylbenzamide |
| 3.7 | 3-(3-chloro-4-((1S,2S)-2-(3-chlorophenyl)cyclopropyl)-5′,6-dimethyl-2-oxo-2H- |
| | [1,4′-bipyridin]-2′-yl)-2-fluoro-N,N-dimethylbenzamide |
| 3.8 | 3-(3-chloro-4-((1S,2S)-2-(2,5-difluorophenyl)cyclopropyl)-5′,6-dimethyl-2-oxo-2H- |
| | [1,4′-bipyridin]-2′-yl)-2-fluoro-N,N-dimethylbenzamide |
| 3.9 | 3-(3-chloro-5′,6-dimethy1-4-((1S,2S)-2-(1-methyl-1H-pyrazol-3-yl)cyclopropyl)-2- |
| | oxo-2H-[1,4′-bipyridin]-2′-yl)-2-fluoro-N,N-dimethylbenzamide |
| 3.10 | 3-(4-((1S,2S)-2-(1-(tert-butyl)-1H-pyrazol-4-yl)cyclopropyl)-3-chloro-5′,6- |
| | dimethyl-2-oxo-2H-[1,4′-bipyridin]-2′-yl)-2-fluoro-N,N-dimethylbenzamide |
| 3.11 | 3-(3-chloro-4-((1S,2S)-2-(5-chloro-3-fluoropyridin-2-yl)cyclopropyl)-5′,6- |
| | dimethyl-2-oxo-2H-[1,4′-bipyridin]-2′-yl)-2-fluoro-N,N-dimethylbenzamide |
| 3.12 | 3-(3-chloro-4-((1S,2S)-2-(5-(difluoromethyl)pyridin-3-yl)cyclopropyl)-5′,6- |
| | dimethyl-2-oxo-2H-[1,4′-bipyridin]-2′-yl)-2-fluoro-N,N-dimethylbenzamide |
| 3.13 | 3-(3,5′-dichloro-4-((1S,2S)-2-(4-fluorophenyl)cyclopropyl)-6-methyl-2-oxo-2H- |
| | [1,4′-bipyridin]-2′-yl)-2-fluoro-N,N-dimethylbenzamide |
| 3.14 | 3-(3,5′-dichloro-4-((1S,2S)-2-(5-fluoropyridin-3-yl)cyclopropyl)-6-methyl-2-oxo- |
| | 2H-[1,4′-bipyridin]-2′-yl)-2-fluoro-N,N-dimethylbenzamide |
| 3.15 | 3-(3,3′-dichloro-4-((1S,2S)-2-(4-fluorophenyl)cyclopropyl)-6-methyl-2-oxo-2H- |
| | [1,4′-bipyridin]-2′-yl)-2-fluoro-N,N-dimethylbenzamide |
| 3.16 | 3-(3,5′-dichloro-4-((1S,2S)-2-(5-chloropyridin-3-yl)cyclopropyl)-6-methyl-2-oxo- |
| | 2H-[1,4′-bipyridin]-2′-yl)-2-fluoro-N,N-dimethylbenzamide |
| 3.17 | 3-(3-chloro-5′,6-dimethyl-2-oxo-4-((1S,2S)-2-(5-(trifluoromethyl)pyridin-3- |
| | yl)cyclopropyl)-2H-[1,4′-bipyridin]-2′-yl)-2-fluoro-N,N-dimethylbenzamide |
| 3.18 | 3-(3-chloro-4-((1S,2S)-2-(5-cyanopyridin-3-yl)cyclopropyl)-5′,6-dimethyl-2-oxo- |
| | 2H-[1,4′-bipyridin]-2′-yl)-2-fluoro-N,N-dimethylbenzamide |
| 3.19 | 3-(3-chloro-4-((1S,2S)-2-(4-fluoro-3-(2-hydroxypropan-2-yl)phenyl)cyclopropyl)- |
| | 5′,6-dimethyl-2-oxo-2H-[1,4′-bipyridin]-2′-yl)-2-fluoro-N,N-dimethylbenzamide |
| 3.20 | 3-(3-chloro-4-((1S,2S)-2-(6-chloropyridin-2-yl)cyclopropyl)-5′,6-dimethyl-2-oxo- |
| | 2H-[1,4′-bipyridin]-2′-yl)-2-fluoro-N,N-dimethylbenzamide |
| 3.21 | N-(3-(3-chloro-4-((1S,2S)-2-(1-(difluoromethyl)-1H-pyrazol-4-yl)cyclopropyl)- |
| | 5′,6-dimethyl-2-oxo-2H-[1,4′-bipyridin]-2′-yl)-2-fluorophenyl)-1- |
| | methylcyclopropane-1-carboxamide |
| 3.22 | N-(3-(3-chloro-5′,6-dimethyl-2-oxo-4-((1S,2S)-2-(5-(trifluoromethyl)pyridin-3- |
| | yl)cyclopropyl)-2H-[1,4′-bipyridin]-2′-yl)-2-fluorophenyl)-1-methylcyclopropane- |
| | 1-carboxamide |
| 3.23 | N-(3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorophenyl)-1-methylcyclopropane-1-carboxamide |
| 3.24 | N-(3-(4-(2-(1H-indazol-1-yl)cyclopropyl)-3-chloro-5′,6-dimethyl-2-oxo-2H-[1,4′- |
| | bipyridin]-2′-yl)-2-fluorophenyl)-1-methylcyclopropane-1-carboxamide |
| 3.25 | 3-(3,5′-dichloro-4-((1S,2S)-2-(5-fluoropyridin-3-yl)cyclopropyl)-6-methyl-2-oxo- |
| | 2H-[1,4′-bipyridin]-2′-yl)-2-fluoro-N-methylbenzamide |
| 3.26 | 3-(3,5′-dichloro-4-((1S,2S)-2-(5-chloropyridin-3-yl)cyclopropyl)-6-methyl-2-oxo- |
| | 2H-[1,4′-bipyridin]-2′-yl)-2-fluoro-N-methylbenzamide |
| 3.27 | N-(3-(3-chloro-3′-fluoro-5′,6-dimethyl-4-((1S,2S)-2-(1-methyl-1H-pyrazol-3- |
| | yl)cyclopropyl)-2-oxo-2H-[1,4′-bipyridin]-2′-yl)-2-fluorophenyl)-1- |
| | methylcyclopropane-1-carboxamide |
| 3.28 | N-(3-(3-chloro-3′-fluoro-4-((1S,2S)-2-(4-fluorophenyl)cyclopropyl)-5′,6-dimethyl- |
| | 2-oxo-2H-[1,4′-bipyridin]-2′-yl)-2-fluorophenyl)-1-methylcyclopropane-1- |
| | carboxamide |
| 3.29 | 3-(3-chloro-3′-fluoro-4-((1S,2S)-2-(5-fluoropyridin-3-yl)cyclopropyl)-5′,6- |
| | dimethyl-2-oxo-2H-[1,4′-bipyridin]-2′-yl)-2-fluoro-N,N-dimethylbenzamide |
| 3.30 | 3′-(3-chloro-4-((1S,2S)-2-(4-fluorophenyl)cyclopropyl)-6-methyl-2-oxopyridin- |
| | 1(2H)-yl)-N-cyclopropyl-2-fluoro-4′-methyl-[1,l′-biphenyl]-3-carboxamide |
| 3.31 | 3-chloro-2′-(3-(1-cyclopropy1-1H-1,2,4-triazol-5-yl)-2-fluorophenyl)-3′-fluoro-5′,6- |
| | dimethy1-4-((1S,2S)-2-(2-(methyl-d3)-2H-1,2,3-triazol-4-yl)cyclopropyl)-2H-[1,4′- |
| | bipyridin]-2-one |
| 3.32 | N-(2-(3-(3-chloro-3′-fluoro-5′,6-dimethyl-4-((1S,2S)-2-(2-(methyl-d3)-2H-1,2,3- |
| | triazol-4-yl)cyclopropyl)-2-oxo-2H-[1,4′-bipyridin]-2′-yl)-2-fluorophenyl)propan-2- |
| | yl)acetamide |
| 3.33 | 3-chloro-4-((1S,2S)-2-(1-(difluoromethyl)-1H-pyrazol-4-yl)cyclopropyl)-3′-fluoro- |
| | 2′-(2-fluoro-3-(3,4,5-trimethyl-1H-pyrazol-1-yl)phenyl)-5′,6-dimethyl-2H-[1,4′- |
| | bipyridin]-2-one |
| 3.34 | 3-chloro-4-((1S,2S)-2-(1-(difluoromethyl)-1H-pyrazol-4-yl)cyclopropyl)-2′-(3-(3,5- |
| | dimethy1-1H-pyrazol-1-yl)-2-fluorophenyl)-3′-fluoro-5′,6-dimethyl-2H-[1,4′- |
| | bipyridin]-2-one |
| 3.35 | 3-chloro-2′-(3-(3,5-dimethyl-1H-1,2,4-triazol-1-yl)-2-fluorophenyl)-3′-fluoro-4- |
| | ((1S,2S)-2-(5-fluoropyridin-3-yl)cyclopropyl)-5′,6-dimethyl-2H-[1,4′-bipyridin]-2- |
| | one |
| 3.36 | 3-chloro-2′-(3-(1-(difluoromethyl)-1H-1,2,4-triazol-5-yl)-2-fluorophenyl)-3′-fluoro- |
| | 5′,6-dimethyl-4-((1S,2S)-2-(1-(methyl-d3)-1H-pyrazol-4-yl)cyclopropyl)-2H-[1,4′- |
| | bipyridin]-2-one |
| 3.37 | 3-chloro-3′-fluoro-2′-(2-fluoro-3-(methylsulfonyl)phenyl)-5′,6-dimethyl-4-((1S,2S)- |
| | 2-(1-(methyl-d3)-1H-pyrazol-3-yl)cyclopropyl)-2H-[1,4′-bipyridin]-2-one |
| 3.38 | 3,5′-dichloro-3′-fluoro-2′-(2-fluoro-3-(methylsulfonyl)phenyl)-6-methyl-4-((1S,2S)- |
| | 2-(2-(methyl-d3)-2H-1,2,3-triazol-4-yl)cyclopropyl)-2H-[1,4′-bipyridin]-2-one |
| 3.39 | 3,3′-dichloro-2′-(2-fluoro-3-(methylsulfonyl)phenyl)-5′,6-dimethyl-4-((1S,2S)-2-(2- |
| | (methyl-d3)-2H-1,2,3-triazol-4-yl)cyclopropyl)-2H-[1,4′-bipyridin]-2-one |
| 4.1 | N-(3-(3-chloro-4-((1S,2S)-2-(5,6-dihydro-4H-pyrrolo[1,2-b]pyrazol-3- |
| | yl)cyclopropyl)-5′,6-dimethyl-2-oxo-2H-[1,4′-bipyridin]-2′-yl)-2-fluorophenyl)-1- |
| | methylcyclopropane-1-carboxamide |
| 4.2 | 3-(3-chloro-5′,6-dimethyl-2-oxo-4-((1S,2S)-2-(pyridin-3-yl)cyclopropyl)-2H-[1,4′- |
| | bipyridin]-2′-yl)-2-fluoro-N,N-dimethylbenzamide |
| 4.3 | 3-(3-chloro-5′,6-dimethyl-2-oxo-4-((1S,2S)-2-(pyridin-4-yl)cyclopropyl)-2H-[1,4′- |
| | bipyridin]-2′-yl)-2-fluoro-N,N-dimethylbenzamide |
| 4.4 | 3-(3-chloro-4-((1S,2S)-2-(5-fluoropyridin-2-yl)cyclopropyl)-5′,6-dimethyl-2-oxo- |
| | 2H-[1,4′-bipyridin]-2′-yl)-2-fluoro-N,N-dimethylbenzamide |
| 4.5 | 3-(3-chloro-4-((1S,2S)-2-(5,6-dihydro-4H-pyrrolo[1,2-b]pyrazol-3-yl)cyclopropyl)- |
| | 5′,6-dimethyl-2-oxo-2H-[1,4′-bipyridin]-2′-yl)-2-fluoro-N,N-dimethylbenzamide |
| 4.6 | 3-(3-chloro-4-((1S,2S)-2-(6,7-dihydro-5H-pyrrolo[1,2-a]imidazol-3- |
| | yl)cyclopropyl)-5′,6-dimethyl-2-oxo-2H-[1,4′-bipyridin]-2′-yl)-2-fluoro-N,N- |
| | dimethylbenzamide |
| 4.7 | 3-(3-chloro-4-((1S,2S)-2-(2,3-dihydropyrazolo[5,1-b]oxazol-7-yl)cyclopropyl)-5′,6- |
| | dimethy1-2-oxo-2H-[1,4′-bipyridin]-2′-yl)-2-fluoro-N,N-dimethylbenzamide |
| 4.8 | 3-(3-chloro-4-((1S,2S)-2-(6,7-dihydro-5H-pyrazolo[5,1-b][1,3]oxazin-3- |
| | yl)cyclopropyl)-5′,6-dimethyl-2-oxo-2H-[1,4′-bipyridin]-2′-yl)-2-fluoro-N,N- |
| | dimethylbenzamide |
| 4.9 | N-(3-(3-chloro-4-((1S,2S)-2-(5-chloropyridin-3-yl)cyclopropyl)-5′,6-dimethyl-2- |
| | oxo-2H-[1,4′-bipyridin]-2′-yl)-2-fluorophenyl)-1-methylcyclopropane-1- |
| | carboxamide |
| 4.10 | 3-(3-chloro-5′,6-dimethyl-2-oxo-4-((1S,2S)-2-(3-oxoisoindolin-4-yl)cyclopropyl)- |
| | 2H-[1,4′-bipyridin]-2′-yl)-2-fluoro-N,N-dimethylbenzamide |
| 4.11 | 3-(3-chloro-5′,6-dimethyl-2-oxo-4-((1S,2S)-2-(1-oxoisoindolin-4-yl)cyclopropyl)- |
| | 2H-[1,4′-bipyridin]-2′-yl)-2-fluoro-N,N-dimethylbenzamide |
| 4.12 | N-(3-(3-chloro-4-((1S,2S)-2-(6-chloropyridazin-4-yl)cyclopropyl)-5′,6-dimethyl-2- |
| | oxo-2H-[1,4′-bipyridin]-2′-yl)-2-fluorophenyl)-1-methylcyclopropane-1- |
| | carboxamide |
| 4.13 | N-(3-(3-chloro-4-((1S,2S)-2-(6,7-dihydro-4H-pyrazolo[5,1-c][1,4]oxazin-2- |
| | yl)cyclopropyl)-5′,6-dimethyl-2-oxo-2H-[1,4′-bipyridin]-2′-yl)-2-fluorophenyl)-1- |
| | methylcyclopropane-1-carboxamide |
| 4.14 | N-(3-(3-chloro-5′,6-dimethyl-4-((1S,2S)-2-(1-methyl-6-oxo-1,6-dihydropyridin-3- |
| | yl)cyclopropyl)-2-oxo-2H-[1,4′-bipyridin]-2′-yl)-2-fluorophenyl)-1- |
| | methylcyclopropane-1-carboxamide |
| 4.15 | N-(3-(3-chloro-4-((1S,2S)-2-(6-chloropyrazin-2-yl)cyclopropyl)-5′,6-dimethyl-2- |
| | oxo-2H-[1,4′-bipyridin]-2′-yl)-2-fluorophenyl)-1-methylcyclopropane-1- |
| | carboxamide |
| 4.16 | 3-(3-chloro-4-((1S,2S)-2-(5-chloro-2-methylpyridin-3-yl)cyclopropyl)-5′,6- |
| | dimethyl-2-oxo-2H-[1,4′-bipyridin]-2′-yl)-2-fluoro-N,N-dimethylbenzamide |
| 4.17 | 3-(3-chloro-4-((1S,2S)-2-(5-fluoro-2-methylpyridin-3-yl)cyclopropyl)-5′,6- |
| | dimethyl-2-oxo-2H-[1,4′-bipyridin]-2′-yl)-2-fluoro-N,N-dimethylbenzamide |
| 4.18 | 3-(4-((1S,2S)-2-(benzo[d]thiazol-4-yl)cyclopropyl)-3-chloro-5′,6-dimethyl-2-oxo- |
| | 2H-[1,4′-bipyridin]-2′-yl)-2-fluoro-N,N-dimethylbenzamide |
| 4.19 | 3-(4-((1S,2S)-2-(benzo[d]thiazol-7-yl)cyclopropyl)-3-chloro-5′,6-dimethyl-2-oxo- |
| | 2H-[1,4′-bipyridin]-2′-yl)-2-fluoro-N,N-dimethylbenzamide |
| 4.20 | 3-(3-chloro-4-((1S,2S)-2-(5-fluoro-6-methylpyridin-2-yl)cyclopropyl)-5′,6- |
| | dimethyl-2-oxo-2H-[1,4′-bipyridin]-2′-yl)-2-fluoro-N,N-dimethylbenzamide |
| 4.21 | 3-(3-chloro-5′,6-dimethy1-4-((1S,2S)-2-(2-methyloxazol-4-yl)cyclopropyl)-2-oxo- |
| | 2H-[1,4′-bipyridin]-2′-yl)-2-fluoro-N,N-dimethylbenzamide |
| 4.22 | 3-(3-chloro-4-((1S,2S)-2-(4-fluoro-1-methyl-1H-pyrazol-3-yl)cyclopropyl)-5′,6- |
| | dimethyl-2-oxo-2H-[1,4′-bipyridin]-2′-yl)-2-fluoro-N,N-dimethylbenzamide |
| 4.23 | N-(3-(3-chloro-4-((1S,2S)-2-(5-chloropyridazin-3-yl)cyclopropyl)-5′,6-dimethyl-2- |
| | oxo-2H-[1,4′-bipyridin]-2′-yl)-2-fluorophenyl)-1-methylcyclopropane-1- |
| | carboxamide |
| 4.24 | 3-(3-chloro-4-((1S,2S)-2-(2-(difluoromethyl)-5-fluoropyridin-3-yl)cyclopropyl)- |
| | 5′,6-dimethyl-2-oxo-2H-[1,4′-bipyridin]-2′-yl)-2-fluoro-N,N-dimethylbenzamide |
| 4.25 | 3-(3-chloro-4-((1S,2S)-2-(5-fluoropyridin-3-yl)cyclopropyl)-5′,6-dimethyl-2-oxo- |
| | 2H-[1,4′-bipyridin]-2′-yl)-2-fluoro-N,N-dimethylbenzamide |
| 4.26 | 3-(3-chloro-4-((1S,2S)-2-(2-cyano-5-fluoropyridin-3-yl)cyclopropyl)-5′,6-dimethyl- |
| | 2-oxo-2H-[1,4′-bipyridin]-2′-yl)-2-fluoro-N,N-dimethylbenzamide |
| 4.27 | 3-(3-chloro-4-((1S,2S)-2-(1-(difluoromethyl)-1H-pyrazol-3-yl)cyclopropyl)-5′,6- |
| | dimethy1-2-oxo-2H-[1,4′-bipyridin]-2′-yl)-2-fluoro-N,N-dimethylbenzamide |
| 4.28 | 3-(3-chloro-4-((1S,2S)-2-(3-(difluoromethyl)phenyl)cyclopropyl)-5′,6-dimethy1-2- |
| | oxo-2H-[1,4′-bipyridin]-2′-yl)-2-fluoro-N,N-dimethylbenzamide |
| 4.29 | N-(3-(3-chloro-4-((1S,2S)-2-(2-chloropyrimidin-4-yl)cyclopropyl)-5′,6-dimethyl-2- |
| | oxo-2H-[1,4′-bipyridin]-2′-yl)-2-fluorophenyl)-1-methylcyclopropane-1- |
| | carboxamide |
| 4.30 | N-(3-(3-chloro-4-((1S,2S)-2-(6-chloropyrimidin-4-yl)cyclopropyl)-5′,6-dimethy1-2- |
| | oxo-2H-[1,4′-bipyridin]-2′-yl)-2-fluorophenyl)-1-methylcyclopropane-1- |
| | carboxamide |
| 4.31 | N-(3-(3-chloro-5′,6-dimethyl-2-oxo-4-((1S,2S)-2-(pyrazolo[1,5-a]pyridin-3- |
| | yl)cyclopropyl)-2H-[1,4′-bipyridin]-2′-yl)-2-fluorophenyl)-1-methylcyclopropane- |
| | 1-carboxamide |
| 4.32 | N-(3-(3-chloro-4-((1S,2S)-2-(5,5-dimethyl-5,6-dihydro-4H-pyrrolo[1,2-b]pyrazol- |
| | 3-yl)cyclopropyl)-5′,6-dimethyl-2-oxo-2H-[1,4′-bipyridin]-2′-yl)-2-fluorophenyl)-1- |
| | methylcyclopropane-1-carboxamide |
| 4.33 | N-(3-(3-chloro-5′,6-dimethyl-4-((1S,2S)-2-(1-methyl-1H-pyrazol-4- |
| | yl)cyclopropyl)-2-oxo-2H-[1,4′-bipyridin]-2′-yl)-2-fluorophenyl)-1- |
| | methylcyclopropane-1-carboxamide |
| 4.34 | N-(3-(3-chloro-4-((1S,2S)-2-(isothiazol-3-yl)cyclopropyl)-5′,6-dimethyl-2-oxo-2H- |
| | [1,4′-bipyridin]-2′-yl)-2-fluorophenyl)-1-methylcyclopropane-1-carboxamide |
| 4.35 | N-(3-(3-chloro-4-((1S,2S)-2-(5-((dimethyl(oxo)-λ⁶-sulfaneylidene)amino)pyridin-3- |
| | yl)cyclopropyl)-5′,6-dimethyl-2-oxo-2H-[1,4′-bipyridin]-2′-yl)-2-fluorophenyl)-1- |
| | methylcyclopropane-1-carboxamide |
| 4.36 | N-(3-(3-chloro-4-((1S,2S)-2-(isothiazol-4-yl)cyclopropyl)-5′,6-dimethyl-2-oxo-2H- |
| | [1,4′-bipyridin]-2′-yl)-2-fluorophenyl)-1-methylcyclopropane-1-carboxamide |
| 4.37 | N-(3-(3-chloro-5′,6-dimethyl-4-((1S,2S)-2-(1-methyl-1H-1,2,3-triazol-4- |
| | yl)cyclopropyl)-2-oxo-2H-[1,4′-bipyridin]-2′-yl)-2-fluorophenyl)-1- |
| | methylcyclopropane-1-carboxamide |
| 4.38 | N-(3-(3-chloro-5′,6-dimethy1-4-((1S,2S)-2-(2-methyl-2H-tetrazol-5- |
| | yl)cyclopropyl)-2-oxo-2H-[1,4′-bipyridin]-2′-yl)-2-fluorophenyl)-1- |
| | methylcyclopropane-1-carboxamide |
| 4.39 | N-(3-(3-chloro-4-((1S,2S)-2-(isothiazol-5-yl)cyclopropyl)-5′,6-dimethyl-2-oxo-2H- |
| | [1,4′-bipyridin]-2′-yl)-2-fluorophenyl)-1-methylcyclopropane-1-carboxamide |
| 4.40 | N-(3-(3-chloro-5′,6-dimethyl-4-((1S,2S)-2-(2-methyl-2H-1,2,3-triazol-4- |
| | yl)cyclopropyl)-2-oxo-2H-[1,4′-bipyridin]-2′-yl)-2-fluorophenyl)-1- |
| | methylcyclopropane-1-carboxamide |
| 4.41 | N-(3-(4-((1S,2S)-2-(1,2,5-thiadiazol-3-yl)cyclopropyl)-3-chloro-5′,6-dimethyl-2- |
| | oxo-2H-[1,4′-bipyridin]-2′-yl)-2-fluorophenyl)-1-methylcyclopropane-1- |
| | carboxamide |
| 4.42 | N-(3-(3-chloro-5′,6-dimethyl-4-((1S,2S)-2-(1-methyl-1,4,5,6- |
| | tetrahydrocyclopenta[c]pyrazol-3-yl)cyclopropyl)-2-oxo-2H-[1,4′-bipyridin]-2′-yl)- |
| | 2-fluorophenyl)-1-methylcyclopropane-1-carboxamide |
| 4.43 | N-(3-(4-((1S,2S)-2-(benzo[d]thiazol-7-yl)cyclopropyl)-3-chloro-5′,6-dimethyl-2- |
| | oxo-2H-[1,4′-bipyridin]-2′-yl)-2-fluorophenyl)-1-methylcyclopropane-1- |
| | carboxamide |
| 4.44 | N-(3-(3-chloro-5′,6-dimethyl-2-oxo-4-((1S,2S)-2-(1-oxoisoindolin-4- |
| | yl)cyclopropyl)-2H-[1,4′-bipyridin]-2′-yl)-2-fluorophenyl)-1-methylcyclopropane- |
| | 1-carboxamide |
| 4.45 | N-(3-(3-chloro-5′,6-dimethyl-2-oxo-4-((1S,2S)-2-(2-oxo-1,2-dihydropyridin-3- |
| | yl)cyclopropyl)-2H-[1,4′-bipyridin]-2′-yl)-2-fluorophenyl)-1-methylcyclopropane- |
| | 1-carboxamide |
| 4.46 | N-(3-(3-chloro-5′,6-dimethyl-4-((1S,2S)-2-(2-methylthiazol-4-yl)cyclopropyl)-2- |
| | oxo-2H-[1,4′-bipyridin]-2′-yl)-2-fluorophenyl)-1-methylcyclopropane-1- |
| | carboxamide |
| 4.47 | N-(3-(3-chloro-5′,6-dimethyl-2-oxo-4-((1S,2S)-2-(2-oxoindolin-4-yl)cyclopropyl)- |
| | 2H-[1,4′-bipyridin]-2′-yl)-2-fluorophenyl)-1-methylcyclopropane-1-carboxamide |
| 4.48 | N-(3-(3-chloro-5′, 6-dimethyl-2-oxo-4-((1S,2S)-2-(2-oxoindolin-6-yl)cyclopropyl)- |
| | 2H-[1,4′-bipyridin]-2′-yl)-2-fluorophenyl)-1-methylcyclopropane-1-carboxamide |
| 4.49 | 3-(3-chloro-4-((1S,2S)-2-(2-chloropyridin-4-yl)cyclopropyl)-5′,6-dimethyl-2-oxo- |
| | 2H-[1,4′-bipyridin]-2′-yl)-2-fluoro-N,N-dimethylbenzamide |
| 4.50 | 3-(3-chloro-4-((1S,2S)-2-(6-chloropyridin-3-yl)cyclopropyl)-5′,6-dimethyl-2-oxo- |
| | 2H-[1,4′-bipyridin]-2′-yl)-2-fluoro-N,N-dimethylbenzamide |
| 4.51 | N-(3-(3-chloro-4-((1S,2S)-2-(6-chloro-3-oxo-2,3-dihydropyridazin-4- |
| | yl)cyclopropyl)-5′,6-dimethyl-2-oxo-2H-[1,4′-bipyridin]-2′-yl)-2-fluorophenyl)-1- |
| | methylcyclopropane-1-carboxamide |
| 4.52 | 3-(3-chloro-4-((1S,2S)-2-(2-(difluoromethyl)pyridin-4-yl)cyclopropyl)-5′,6- |
| | dimethyl-2-oxo-2H-[1,4′-bipyridin]-2′-yl)-2-fluoro-N,N-dimethylbenzamide |
| 4.53 | 3-(3-chloro-4-((1S,2S)-2-(2-methoxypyridin-4-yl)cyclopropyl)-5′,6-dimethyl-2- |
| | oxo-2H-[1,4′-bipyridin]-2′-yl)-2-fluoro-N,N-dimethylbenzamide |
| 4.54 | 3-(3-chloro-4-((1S,2S)-2-(6-fluoropyridin-3-yl)cyclopropyl)-5′,6-dimethyl-2-oxo- |
| | 2H-[1,4′-bipyridin]-2′-yl)-2-fluoro-N,N-dimethylbenzamide |
| 4.55 | 3-(3-chloro-4-((1S,2S)-2-(2-fluoropyridin-4-yl)cyclopropyl)-5′,6-dimethyl-2-oxo- |
| | 2H-[1,4′-bipyridin]-2′-yl)-2-fluoro-N,N-dimethylbenzamide |
| 4.56 | 3-chloro-2′-(2-fluoro-3-(methylsulfonyl)phenyl)-5′,6-dimethyl-4-((1S,2S)-2-(2-oxo- |
| | 1,2-dihydropyridin-3-yl)cyclopropyl)-2H-[1,4′-bipyridin]-2-one |
| 4.57 | 3-chloro-2′-(2-fluoro-3-(methylsulfonyl)phenyl)-5′,6-dimethyl-4-((1S,2S)-2-(5- |
| | methyl-2-oxo-1,2-dihydropyridin-3-yl)cyclopropyl)-2H-[1,4′-bipyridin]-2-one |
| 4.58 | 3-chloro-4-((1S,2S)-2-(5-fluoro-2-oxo-1,2-dihydropyridin-3-yl)cyclopropyl)-2′-(2- |
| | fluoro-3-(methylsulfonyl)phenyl)-5′,6-dimethyl-2H-[1,4′-bipyridin]-2-one |
| 4.59 | 3-chloro-2′-(2-fluoro-3-(methylsulfonyl)phenyl)-5′,6-dimethyl-4-((1S,2S)-2-(2-oxo- |
| | 5-(trifluoromethyl)-1,2-dihydropyridin-3-yl)cyclopropyl)-2H-[1,4′-bipyridin]-2-one |
| 4.60 | 3-chloro-3′-fluoro-2′-(2-fluoro-3-(methylsulfonyl)phenyl)-5′,6-dimethyl-4-((1S,2S)- |
| | 2-(2-oxo-1,2-dihydropyridin-3-yl)cyclopropyl)-2H-[1,4′-bipyridin]-2-one |
| 4.61 | 3-chloro-3′-fluoro-2′-(2-fluoro-3-(methylsulfonyl)phenyl)-5′,6-dimethyl-4-((1S,2S)- |
| | 2-(6-oxo-1,6-dihydropyridin-2-yl)cyclopropyl)-2H-[1,4′-bipyridin]-2-one |
| 4.62 | 3-chloro-4-((1S,2S)-2-(5-(difluoromethyl)pyridin-3-yl)cyclopropyl)-3′-fluoro-2′-(2- |
| | fluoro-3-(methylsulfonyl)phenyl)-5′,6-dimethyl-2H-[1,4′-bipyridin]-2-one |
| 4.63 | 3-chloro-3′-fluoro-2′-(2-fluoro-3-(methylsulfonyl)phenyl)-5′,6-dimethyl-4-((1S,2S)- |
| | 2-(5-(trifluoromethyl)pyridin-3-yl)cyclopropyl)-2H-[1,4′-bipyridin]-2-one |
| 4.64 | 3-chloro-3′-fluoro-2′-(2-fluoro-3-(methylsulfonyl)phenyl)-4-((1S,2S)-2-(5-fluoro-4- |
| | methylpyrimidin-2-yl)cyclopropyl)-5′,6-dimethyl-2H-[1,4′-bipyridin]-2-one |
| 4.65 | 3-chloro-3′-fluoro-2′-(2-fluoro-3-(methylsulfonyl)phenyl)-5′,6-dimethyl-4-((1S,2S)- |
| | 2-(1-(methyl-d3)-1H-pyrazol-3-yl)cyclopropyl)-2H-[1,4′-bipyridin]-2-one |
| 4.66 | 3-chloro-3′-fluoro-2′-(2-fluoro-3-(methylsulfonyl)phenyl)-5′,6-dimethyl-4-((1S,2S)- |
| | 2-(6-methylpyrazin-2-yl)cyclopropyl)-2H-[1,4′-bipyridin]-2-one |
| 4.67 | 3-chloro-3′-fluoro-2′-(2-fluoro-3-(methylsulfonyl)phenyl)-4-((1S,2S)-2-(4- |
| | fluoropyrazolo[1,5-a]pyridin-7-yl)cyclopropyl)-5′,6-dimethyl-2H-[1,4′-bipyridin]-2- |
| | one |
| 4.68 | 3-chloro-3′-fluoro-2′-(2-fluoro-3-(methylsulfonyl)phenyl)-5′,6-dimethyl-4-((1S,2S)- |
| | 2-(1-(methyl-d3)-1H-pyrazol-3-yl)cyclopropyl)-2H-[1,4′-bipyridin]-2-one |
| 4.69 | 3-chloro-3′-fluoro-2′-(2-fluoro-3-(methylsulfonyl)phenyl)-5′,6-dimethyl-4-((1S,2S)- |
| | 2-(pyrimidin-4-yl)cyclopropyl)-2H-[1,4′-bipyridin]-2-one |
| 4.70 | 3-chloro-4-((1S,2S)-2-(2,5-dimethyl-2H-1,2,3-triazol-4-yl)cyclopropyl)-3′-fluoro- |
| | 2′-(2-fluoro-3-(methylsulfonyl)phenyl)-5′,6-dimethyl-2H-[1,4′-bipyridin]-2-one |
| 4.71 | 3-chloro-3′-fluoro-2′-(2-fluoro-3-(methylsulfonyl)phenyl)-4-((1S,2S)-2-(5- |
| | fluoropyrimidin-2-yl)cyclopropyl)-5′,6-dimethyl-2H-[1,4′-bipyridin]-2-one |
| 4.72 | 3-chloro-3′-fluoro-2′-(2-fluoro-3-(methylsulfonyl)phenyl)-5′,6-dimethyl-4-((1S,2S)- |
| | 2-(4-(methyl-d3)pyrimidin-2-yl)cyclopropyl)-2H-[1,4′-bipyridin]-2-one |
| 4.73 | 3-chloro-3′-fluoro-2′-(2-fluoro-3-(methylsulfonyl)phenyl)-5′,6-dimethyl-4-((1S,2S)- |
| | 2-(pyrimidin-5-yl)cyclopropyl)-2H-[1,4′-bipyridin]-2-one |
| 4.74 | 3-((1S,2S)-2-(3-chloro-3′-fluoro-2′-(2-fluoro-3-(methylsulfonyl)phenyl)-5′,6- |
| | dimethyl-2-oxo-2H-[1,4′-bipyridin]-4-yl)cyclopropyl)-1-methyl-1H-pyrazole-5- |
| | carbonitrile |
| 4.75 | 3-chloro-4-((1S,2S)-2-(1,5-dimethyl-1H-pyrazol-3-yl)cyclopropyl)-3′-fluoro-2′-(2- |
| | fluoro-3-(methylsulfonyl)phenyl)-5′,6-dimethyl-2H-[1,4′-bipyridin]-2-one |
| 4.76 | 3-chloro-4-((1S,2S)-2-(1,5-dimethyl-1H-pyrazol-4-yl)cyclopropyl)-3′-fluoro-2′-(2- |
| | fluoro-3-(methylsulfonyl)phenyl)-5′,6-dimethyl-2H-[1,4′-bipyridin]-2-one |
| 4.77 | 3-chloro-3′-fluoro-2′-(2-fluoro-3-(methylsulfonyl)phenyl)-5′,6-dimethyl-4-((1S,2S)- |
| | 2-(pyridazin-4-yl)cyclopropyl)-2H-[1,4′-bipyridin]-2-one |
| 4.78 | 3-chloro-3′-fluoro-2′-(2-fluoro-3-(methylsulfonyl)phenyl)-5′,6-dimethyl-4-((1S,2S)- |
| | 2-(6-methylpyridin-2-yl)cyclopropyl)-2H-[1,4′-bipyridin]-2-one |
| 4.79 | 3-chloro-3′-fluoro-2′-(2-fluoro-3-(methylsulfonyl)phenyl)-4-((1S,2S)-2-(5- |
| | fluoropyrimidin-4-yl)cyclopropyl)-5′,6-dimethyl-2H-[1,4′-bipyridin]-2-one |
| 4.80 | 3-chloro-3′-fluoro-2′-(2-fluoro-3-(methylsulfonyl)phenyl)-5′,6-dimethyl-4-((1S,2S)- |
| | 2-(pyridazin-3-yl)cyclopropyl)-2H-[1,4′-bipyridin]-2-one |
| 4.81 | 3-chloro-4-((1S,2S)-2-(1-cyclopropyl-1H-pyrazol-4-yl)cyclopropyl)-3′-fluoro-2′-(2- |
| | fluoro-3-(methylsulfonyl)phenyl)-5′,6-dimethyl-2H-[1,4′-bipyridin]-2-one |
| 4.82 | 3-chloro-3′-fluoro-4-((1S,2S)-2-(5-fluoro-1-methyl-1H-pyrazol-4-yl)cyclopropyl)- |
| | 2′-(2-fluoro-3-(methylsulfonyl)phenyl)-5′,6-dimethyl-2H-[1,4′-bipyridin]-2-one |
| 4.83 | 3-chloro-3′-fluoro-2′-(2-fluoro-3-(methylsulfonyl)phenyl)-5′,6-dimethyl-4-((1S,2S)- |
| | 2-(1-methyl-6-oxo-1,6-dihydropyridin-2-yl)cyclopropyl)-2H-[1,4′-bipyridin]-2-one |
| 4.84 | 3-chloro-4-((1S,2S)-2-(1-(difluoromethyl)-1H-1,2,3-triazol-4-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.85 | 3-chloro-4-((1S,2S)-2-(2-(difluoromethyl)-2H-1,2,3-triazol-4-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.86 | 3-chloro-4-((1S,2S)-2-(2-(cyclopropylmethyl)-2H-1,2,3-triazol-4-yl)cyclopropyl)- |
| | 3′-fluoro-2′-(2-fluoro-3-(methylsulfonyl)phenyl)-5′,6-dimethyl-2H-[1,4′-bipyridin]- |
| | 2-one |
| 4.87 | 3-chloro-3′-fluoro-2′-(2-fluoro-3-(methylsulfonyl)phenyl)-4-((1S,2S)-2-(2- |
| | isopropyl-2H-1,2,3-triazol-4-yl)cyclopropyl)-5′,6-dimethyl-2H-[1,4′-bipyridin]-2- |
| | one |
| 4.88 | 3-chloro-3′-fluoro-2′-(2-fluoro-3-(methylsulfonyl)phenyl)-5′,6-dimethyl-4-((1S,2S)- |
| | 2-(2-propyl-2H-1,2,3-triazol-4-yl)cyclopropyl)-2H-[1,4′-bipyridin]-2-one |
| 4.89 | 3-chloro-4-((1S,2S)-2-(2-ethyl-2H-1,2,3-triazol-4-yl)cyclopropyl)-3′-fluoro-2′-(2- |
| | fluoro-3-(methylsulfonyl)phenyl)-5′,6-dimethyl-2H-[1,4′-bipyridin]-2-one |
| 4.90 | 3-chloro-3′-fluoro-2′-(2-fluoro-3-(methylsulfonyl)phenyl)-5′,6-dimethyl-4-((1S,2S)- |
| | 2-(2-methylthiazol-4-yl)cyclopropyl)-2H-[1,4′-bipyridin]-2-one |
| 4.91 | 3-chloro-3′-fluoro-2′-(2-fluoro-3-(methylsulfonyl)phenyl)-4-((1S,2S)-2-(isothiazol- |
| | 3-yl)cyclopropyl)-5′,6-dimethyl-2H-[1,4′-bipyridin]-2-one |
| 4.92 | 3-chloro-4-((1S,2S)-2-(1-cyclopropyl-1H-pyrazol-5-yl)cyclopropyl)-3′-fluoro-2′-(2- |
| | fluoro-3-(methylsulfonyl)phenyl)-5′,6-dimethyl-2H-[1,4′-bipyridin]-2-one |
| 4.93 | 3-chloro-3′-fluoro-4-((1S,2S)-2-(3-fluoro-1-methyl-1H-pyrazol-5-yl)cyclopropyl)- |
| | 2′-(2-fluoro-3-(methylsulfonyl)phenyl)-5′,6-dimethyl-2H-[1,4′-bipyridin]-2-one |
| 4.94 | 2-(4-((1S,2S)-2-(3-chloro-3′-fluoro-2′-(2-fluoro-3-(methylsulfonyl)phenyl)-5′,6- |
| | dimethyl-2-oxo-2H-[1,4′-bipyridin]-4-yl)cyclopropyl)-1H-pyrazol-1-yl)acetonitrile |
| 4.95 | 4-((1S,2S)-2-(3-chloro-3′-fluoro-2′-(2-fluoro-3-(methylsulfonyl)phenyl)-5′,6- |
| | dimethy1-2-oxo-2H-[1,4′-bipyridin]-4-yl)cyclopropyl)-1-methyl-1H-pyrazole-3- |
| | carbonitrile |
| 4.96 | 3-chloro-3′-fluoro-2′-(2-fluoro-3-(methylsulfonyl)phenyl)-5′,6-dimethyl-4-((1S,2S)- |
| | 2-(2-methyl-2H-indazol-3-yl)cyclopropyl)-2H-[1,4′-bipyridin]-2-one |
| 4.97 | 3-(4-((1S,2S)-2-(3-chloro-3′-fluoro-2′-(2-fluoro-3-(methylsulfonyl)phenyl)-5′,6- |
| | dimethy1-2-oxo-2H-[1,4′-bipyridin]-4-yl)cyclopropyl)-1H-pyrazol-1- |
| | yl)propanenitrile |
| 4.98 | 4-((1S,2S)-2-(3-chloro-3′-fluoro-2′-(2-fluoro-3-(methylsulfonyl)phenyl)-5′,6- |
| | dimethyl-2-oxo-2H-[1,4′-bipyridin]-4-yl)cyclopropyl)-1-methyl-1H-pyrazole-5- |
| | carbonitrile |
| 4.99 | 3-chloro-3′-fluoro-2′-(2-fluoro-3-(methylsulfonyl)phenyl)-5′,6-dimethyl-4-((1S,2S)- |
| | 2-(pyrazin-2-yl)cyclopropyl)-2H-[1,4′-bipyridin]-2-one |
| 4.100 | 4-((1S,2S)-2-(3-chloro-3′-fluoro-2′-(2-fluoro-3-(methylsulfonyl)phenyl)-5′,6- |
| | dimethy1-2-oxo-2H-[1,4′-bipyridin]-4-yl)cyclopropyl)isoindoline-1,3-dione |
| 4.101 | 3-chloro-4-((1S,2S)-2-(6-chloro-3-oxo-3,4-dihydropyrazin-2-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.102 | 3-((1S,2S)-2-(3-chloro-3′-fluoro-2′-(2-fluoro-3-(methylsulfonyl)phenyl)-5′,6- |
| | dimethy1-2-oxo-2H-[1,4′-bipyridin]-4-yl)cyclopropyl)-1-methyl-1H-pyrazole-5- |
| | carboxamide |
| 4.103 | 3-chloro-3′-fluoro-2′-(2-fluoro-3-(methylsulfonyl)phenyl)-5′,6-dimethyl-4-((1S,2S)- |
| | 2-(pyrimidin-2-yl)cyclopropyl)-2H-[1,4′-bipyridin]-2-one |
| 4.104 | 3-chloro-3′-fluoro-2′-(2-fluoro-3-(methylsulfonyl)phenyl)-5′,6-dimethyl-4-((1S,2S)- |
| | 2-(4-methylpyrimidin-2-yl)cyclopropyl)-2H-[1,4′-bipyridin]-2-one |
| 4.105 | 3-chloro-3′-fluoro-2′-(2-fluoro-3-(methylsulfonyl)phenyl)-5′,6-dimethyl-4-((1S,2S)- |
| | 2-(6-methyl-2-oxo-1,2-dihydropyridin-3-yl)cyclopropyl)-2H-[1,4′-bipyridin]-2-one |
| 4.106 | 3-chloro-3′-fluoro-2′-(2-fluoro-3-(methylsulfonyl)phenyl)-5′,6-dimethyl-4-((1S,2S)- |
| | 2-(2-(methyl-d3)-2H-1,2,3-triazol-4-yl)cyclopropyl)-2H-[1,4′-bipyridin]-2-one |
| 4.107 | 4-((1S,2S)-2-(6-aminopyridin-2-yl)cyclopropyl)-3-chloro-3′-fluoro-2′-(2-fluoro-3- |
| | (methylsulfonyl)phenyl)-5′,6-dimethyl-2H-[1,4′-bipyridin]-2-one |
| 4.108 | 3-chloro-3′-fluoro-2′-(2-fluoro-3-(methylsulfonyl)phenyl)-4-((1S,2S)-2- |
| | (imidazo[2,1-b]thiazol-3-yl)cyclopropyl)-5′,6-dimethyl-2H-[1,4′-bipyridin]-2-one |
| 4.109 | 4-((1S,2S)-2-(1H-pyrazol-4-yl)cyclopropyl)-3-chloro-3′-fluoro-2′-(2-fluoro-3- |
| | (methylsulfonyl)phenyl)-5′,6-dimethyl-2H-[1,4′-bipyridin]-2-one |
| 4.110 | 3-chloro-4-((1S,2S)-2-(3,5-difluoropyridin-2-yl)cyclopropyl)-3′-fluoro-2′-(2-fluoro- |
| | 3-(methylsulfonyl)phenyl)-5′,6-dimethyl-2H-[1,4′-bipyridin]-2-one |
| 4.111 | 3-chloro-3′-fluoro-2′-(2-fluoro-3-(methylsulfonyl)phenyl)-5′,6-dimethyl-4-((1S,2S)- |
| | 2-(1-(methyl-d3)-1H-pyrazol-4-yl)cyclopropyl)-2H-[1,4′-bipyridin]-2-one |
| 4.112 | 3-chloro-3′-fluoro-2′-(2-fluoro-3-(methylsulfonyl)phenyl)-5′,6-dimethyl-4-((1S,2S)- |
| | 2-(3-oxo-3,4-dihydropyrazin-2-yl)cyclopropyl)-2H-[1,4′-bipyridin]-2-one |
| 4.113 | 3-chloro-3′-fluoro-2′-(2-fluoro-3-(methylsulfonyl)phenyl)-4-((1S,2S)-2-(3-fluoro-6- |
| | oxo-1,6-dihydropyridin-2-yl)cyclopropyl)-5′,6-dimethyl-2H-[1,4′-bipyridin]-2-one |
| 4.114 | 3-chloro-3′-fluoro-4-((1S,2S)-2-(4-fluoro-2-oxo-1,2-dihydropyridin-3- |
| | yl)cyclopropyl)-2′-(2-fluoro-3-(methylsulfonyl)phenyl)-5′,6-dimethyl-2H-[1,4′- |
| | bipyridin]-2-one |
| 4.115 | 3-chloro-3′-fluoro-2′-(2-fluoro-3-(methylsulfonyl)phenyl)-4-((1S,2S)-2-(2-fluoro-6- |
| | oxo-1,6-dihydropyridin-3-yl)cyclopropyl)-5′,6-dimethyl-2H-[1,4′-bipyridin]-2-one |
| 4.116 | 3-chloro-3′-fluoro-2′-(2-fluoro-3-(methylsulfonyl)phenyl)-5′,6-dimethyl-4-((1S,2S)- |
| | 2-(5-methyl-2-oxo-1,2-dihydropyridin-3-yl)cyclopropyl)-2H-[1,4′-bipyridin]-2-one |
| 4.117 | 3-chloro-3′-fluoro-2′-(2-fluoro-3-(methylsulfonyl)phenyl)-5′,6-dimethyl-4-((1S,2S)- |
| | 2-(pyrazolo[1,5-a]pyridin-6-yl)cyclopropyl)-2H-[1,4′-bipyridin]-2-one |
| 4.118 | 3-chloro-3′-fluoro-2′-(2-fluoro-3-(methylsulfonyl)phenyl)-4-((1S,2S)-2-(6-fluoro- |
| | [1,2,4]triazolo[1,5-a]pyridin-8-yl)cyclopropyl)-5′,6-dimethyl-2H-[1,4′-bipyridin]-2- |
| | one |
| 4.119 | 3-chloro-4-((1S,2S)-2-(6-chloroimidazo[1,2-b]pyridazin-8-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.120 | 3-chloro-3′-fluoro-2′-(2-fluoro-3-(methylsulfonyl)phenyl)-4-((1S,2S)-2-(6- |
| | fluoropyrazolo[1,5-a]pyridin-4-yl)cyclopropyl)-5′,6-dimethyl-2H-[1,4′-bipyridin]-2- |
| | one |
| 4.121 | 4-((1S,2S)-2-(3-chloro-3′-fluoro-2′-(2-fluoro-3-(methylsulfonyl)phenyl)-5′,6- |
| | dimethy1-2-oxo-2H-[1,4′-bipyridin]-4-yl)cyclopropyl)pyrazolo[1,5-a]pyridine-3- |
| | carbonitrile |
| 4.122 | 4-((1S,2S)-2-([1,2,4]triazolo[4,3-a]pyridin-8-yl)cyclopropyl)-3-chloro-3′-fluoro-2′- |
| | (2-fluoro-3-(methylsulfonyl)phenyl)-5′,6-dimethyl-2H-[1,4′-bipyridin]-2-one |
| 4.123 | 3-chloro-3′-fluoro-2′-(2-fluoro-3-(methylsulfonyl)phenyl)-4-((1S,2S)-2- |
| | (imidazo[1,5-a]pyridin-6-yl)cyclopropyl)-5′,6-dimethy1-2H-[1,4′-bipyridin]-2-one |
| 4.124 | 3-chloro-3′-fluoro-2′-(2-fluoro-3-(methylsulfonyl)phenyl)-5′,6-dimethyl-4-((1S,2S)- |
| | 2-(pyrazolo[1,5-a]pyridin-5-yl)cyclopropyl)-2H-[1,4′-bipyridin]-2-one |
| 4.125 | 3-chloro-3′-fluoro-2′-(2-fluoro-3-(methylsulfonyl)phenyl)-5′,6-dimethyl-4-((1S,2S)- |
| | 2-(pyrrolo[1,2-a]pyrimidin-3-yl)cyclopropyl)-2H-[1,4′-bipyridin]-2-one |
| 4.126 | 3-chloro-4-((1S,2S)-2-(5,6-dihydro-4H-pyrrolo[1,2-b]pyrazol-3-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.127 | 3-chloro-3′-fluoro-4-((1S,2S)-2-(3-fluoro-2-oxo-1,2-dihydropyridin-4- |
| | yl)cyclopropyl)-2′-(2-fluoro-3-(methylsulfonyl)phenyl)-5′,6-dimethyl-2H-[1,4′- |
| | bipyridin]-2-one |
| 4.128 | 3-chloro-4-((1S,2S)-2-(5-chloro-2-oxo-1,2-dihydropyridin-3-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.129 | 3-chloro-3′-fluoro-2′-(2-fluoro-3-(methylsulfonyl)phenyl)-4-((1S,2S)-2- |
| | (imidazo[1,2-b]pyridazin-3-yl)cyclopropyl)-5′,6-dimethyl-2H-[1,4′-bipyridin]-2-one |
| 4.130 | 3-chloro-4-((1S,2S)-2-(6-chloro-3-oxo-2,3-dihydropyridazin-4-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.131 | 3-chloro-3′-fluoro-4-((1S,2S)-2-(5-fluoro-2-oxo-1,2-dihydropyridin-3- |
| | yl)cyclopropyl)-2′-(2-fluoro-3-(methylsulfonyl)phenyl)-5′,6-dimethyl-2H-[1,4′- |
| | bipyridin]-2-one |
| 4.132 | 3-chloro-3′-fluoro-4-((1S,2S)-2-(4-fluoro-3-(2-hydroxypropan-2- |
| | yl)phenyl)cyclopropyl)-2′-(2-fluoro-3-(methylsulfonyl)phenyl)-5′,6-dimethyl-2H- |
| | [1,4′-bipyridin]-2-one |
| 4.133 | 4-((1S,2S)-2-(benzo[d]thiazol-4-yl)cyclopropyl)-3-chloro-3′-fluoro-2′-(2-fluoro-3- |
| | (methylsulfonyl)phenyl)-5′,6-dimethyl-2H-[1,4′-bipyridin]-2-one |
| 4.134 | 4-((1S,2S)-2-(2-aminopyridin-3-yl)cyclopropyl)-3-chloro-3′-fluoro-2′-(2-fluoro-3- |
| | (methylsulfonyl)phenyl)-5′,6-dimethyl-2H-[1,4′-bipyridin]-2-one |
| 4.135 | 3-chloro-3′-fluoro-2′-(2-fluoro-3-(methylsulfonyl)phenyl)-5′,6-dimethyl-4-((1S,2S)- |
| | 2-(pyrazolo[1,5-a]pyridin-3-yl)cyclopropyl)-2H-[1,4′-bipyridin]-2-one |
| 4.136 | 3-chloro-3′-fluoro-2′-(2-fluoro-3-(methylsulfonyl)phenyl)-5′,6-dimethyl-4-((1S,2S)- |
| | 2-(pyrazolo[1,5-a]pyrimidin-3-yl)cyclopropyl)-2H-[1,4′-bipyridin]-2-one |
| 4.137 | 3-chloro-3′-fluoro-2′-(2-fluoro-3-(methylsulfonyl)phenyl)-5′,6-dimethyl-4-((1S,2S)- |
| | 2-(pyrazolo[1,5-a]pyridin-4-yl)cyclopropyl)-2H-[1,4′-bipyridin]-2-one |
| 4.138 | 3-chloro-3′-fluoro-2′-(2-fluoro-3-(methylsulfonyl)phenyl)-5′,6-dimethyl-4-((1S,2S)- |
| | 2-(2-oxo-1,2-dihydropyridin-4-yl)cyclopropyl)-2H-[1,4′-bipyridin]-2-one |
| 4.139 | 3-chloro-3′-fluoro-2′-(2-fluoro-3-(methylsulfonyl)phenyl)-5′,6-dimethyl-4-((1S,2S)- |
| | 2-(6-oxo-1,6-dihydropyridin-3-yl)cyclopropyl)-2H-[1,4′-bipyridin]-2-one |
| 4.140 | 3-chloro-3′-fluoro-2′-(2-fluoro-3-(methylsulfonyl)phenyl)-4-((1S,2S)-2-(6- |
| | fluoropyridin-3-yl)cyclopropyl)-5′,6-dimethyl-2H-[1,4′-bipyridin]-2-one |
| 4.141 | 3-chloro-4-((1S,2S)-2-(2,3-difluorophenyl)cyclopropyl)-3′-fluoro-2′-(2-fluoro-3- |
| | (methylsulfonyl)phenyl)-5′,6-dimethyl-2H-[1,4′-bipyridin]-2-one |
| 4.142 | 3-chloro-4-((1S,2S)-2-(3-chloropyridin-2-yl)cyclopropyl)-3′-fluoro-2′-(2-fluoro-3- |
| | (methylsulfonyl)phenyl)-5′,6-dimethyl-2H-[1,4′-bipyridin]-2-one |
| 4.143 | 3-chloro-3′-fluoro-2′-(2-fluoro-3-(methylsulfonyl)phenyl)-4-((1S,2S)-2-(3- |
| | fluoropyridin-2-yl)cyclopropyl)-5′,6-dimethyl-2H-[1,4′-bipyridin]-2-one |
| 4.144 | 3-chloro-4-((1S,2S)-2-(4-chloropyridin-3-yl)cyclopropyl)-3′-fluoro-2′-(2-fluoro-3- |
| | (methylsulfonyl)phenyl)-5′,6-dimethyl-2H-[1,4′-bipyridin]-2-one |
| 4.145 | 3-chloro-4-((1S,2S)-2-(4-chloropyridin-2-yl)cyclopropyl)-3′-fluoro-2′-(2-fluoro-3- |
| | (methylsulfonyl)phenyl)-5′,6-dimethyl-2H-[1,4′-bipyridin]-2-one |
| 4.146 | 3-chloro-3′-fluoro-2′-(2-fluoro-3-(methylsulfonyl)phenyl)-4-((1S,2S)-2-(3- |
| | fluoropyridin-4-yl)cyclopropyl)-5′,6-dimethyl-2H-[1,4′-bipyridin]-2-one |
| 4.147 | 3-chloro-4-((1S,2S)-2-(1-cyclopropyl-1H-pyrazol-3-yl)cyclopropyl)-3′-fluoro-2′-(2- |
| | fluoro-3-(methylsulfonyl)phenyl)-5′,6-dimethyl-2H-[1,4′-bipyridin]-2-one |
| 4.148 | 3-chloro-3′-fluoro-4-((1S,2S)-2-(3-fluoro-1-methyl-1H-pyrazol-4-yl)cyclopropyl)- |
| | 2′-(2-fluoro-3-(methylsulfonyl)phenyl)-5′,6-dimethyl-2H-[1,4′-bipyridin]-2-one |
| 4.149 | 3-chloro-4-((1S,2S)-2-(1,4-dimethyl-1H-pyrazol-3-yl)cyclopropyl)-3′-fluoro-2′-(2- |
| | fluoro-3-(methylsulfonyl)phenyl)-5′,6-dimethy1-2H-[1,4′-bipyridin]-2-one |
| 4.150 | 3-chloro-3′-fluoro-2′-(2-fluoro-3-(methylsulfonyl)phenyl)-5′,6-dimethyl-4-((1S,2S)- |
| | 2-(pyridin-3-yl)cyclopropyl)-2H-[1,4′-bipyridin]-2-one |
| 4.151 | 3-chloro-3′-fluoro-2′-(2-fluoro-3-(methylsulfonyl)phenyl)-5′,6-dimethyl-4-((1S,2S)- |
| | 2-(1-methyl-1H-pyrazol-5-yl)cyclopropyl)-2H-[1,4′-bipyridin]-2-one |
| 4.152 | 3-((1S,2S)-2-(3-chloro-3′-fluoro-2′-(2-fluoro-3-(methylsulfonyl)phenyl)-5′,6- |
| | dimethy1-2-oxo-2H-[1,4′-bipyridin]-4-yl)cyclopropyl)-1-methyl-1H-pyrazole-4- |
| | carbonitrile |
| 4.153 | 3-chloro-4-((1S,2S)-2-(6-chloropyridin-2-yl)cyclopropyl)-3′-fluoro-2′-(2-fluoro-3- |
| | (methylsulfonyl)phenyl)-5′,6-dimethyl-2H-[1,4′-bipyridin]-2-one |
| 4.154 | 3-chloro-3′-fluoro-2′-(2-fluoro-3-(methylsulfonyl)phenyl)-5′,6-dimethyl-4-((1S,2S)- |
| | 2-(pyridin-2-yl)cyclopropyl)-2H-[1,4′-bipyridin]-2-one |
| 4.155 | 3-chloro-3′-fluoro-2′-(2-fluoro-3-(methylsulfonyl)phenyl)-4-((1S,2S)-2-(5- |
| | fluoropyridin-2-yl)cyclopropyl)-5′,6-dimethyl-2H-[1,4′-bipyridin]-2-one |
| 4.156 | 3-chloro-4-((1S,2S)-2-(3-chloropyridin-4-yl)cyclopropyl)-3′-fluoro-2′-(2-fluoro-3- |
| | (methylsulfonyl)phenyl)-5′,6-dimethyl-2H-[1,4′-bipyridin]-2-one |
| 4.157 | 3-chloro-4-((1S,2S)-2-(1-(difluoromethyl)-1H-pyrazol-3-yl)cyclopropyl)-3′-fluoro- |
| | 2′-(2-fluoro-3-(methylsulfonyl)phenyl)-5′,6-dimethyl-2H-[1,4′-bipyridin]-2-one |
| 4.158 | 3-chloro-3′-fluoro-2′-(2-fluoro-3-(methylsulfonyl)phenyl)-5′,6-dimethyl-4-((1S,2S)- |
| | 2-(1-methyl-1H-pyrazol-3-yl)cyclopropyl)-2H-[1,4′-bipyridin]-2-one |
| 4.159 | 3-chloro-4-((1S,2S)-2-(6-(difluoromethyl)pyridin-2-yl)cyclopropyl)-3′-fluoro-2′-(2- |
| | fluoro-3-(methylsulfonyl)phenyl)-5′,6-dimethy1-2H-[1,4′-bipyridin]-2-one |
| 4.160 | 3-chloro-3′-fluoro-2′-(2-fluoro-3-(methylsulfonyl)phenyl)-5′,6-dimethyl-4-((1S,2S)- |
| | 2-(6-(trifluoromethyl)pyrazin-2-yl)cyclopropyl)-2H-[1,4′-bipyridin]-2-one |
| 4.161 | 3-chloro-4-((1S,2S)-2-(3,5-difluorophenyl)cyclopropyl)-3′-fluoro-2′-(2-fluoro-3- |
| | (methylsulfonyl)phenyl)-5′,6-dimethyl-2H-[1,4′-bipyridin]-2-one |
| 4.162 | 3-chloro-4-((1S,2S)-2-(2,4-difluorophenyl)cyclopropyl)-3′-fluoro-2′-(2-fluoro-3- |
| | (methylsulfonyl)phenyl)-5′,6-dimethyl-2H-[1,4′-bipyridin]-2-one |
| 4.163 | 3-chloro-4-((1S,2S)-2-(2,6-difluorophenyl)cyclopropyl)-3′-fluoro-2′-(2-fluoro-3- |
| | (methylsulfonyl)phenyl)-5′,6-dimethyl-2H-[1,4′-bipyridin]-2-one |
| 4.164 | 3-chloro-3′-fluoro-2′-(2-fluoro-3-(methylsulfonyl)phenyl)-4-((1S,2S)-2-(2- |
| | fluorophenyl)cyclopropyl)-5′,6-dimethyl-2H-[1,4′-bipyridin]-2-one |
| 4.165 | 3-chloro-3′-fluoro-2′-(2-fluoro-3-(methylsulfonyl)phenyl)-4-((1S,2S)-2-(3- |
| | fluorophenyl)cyclopropyl)-5′,6-dimethyl-2H-[1,4′-bipyridin]-2-one |
| 4.166 | 3-chloro-3′-fluoro-2′-(2-fluoro-3-(methylsulfonyl)phenyl)-5′,6-dimethyl-4-((1S,2S)- |
| | 2-(1-methyl-1H-1,2,3-triazol-4-yl)cyclopropyl)-2H-[1,4′-bipyridin]-2-one |
| 4.167 | 3-chloro-3′-fluoro-2′-(2-fluoro-3-(methylsulfonyl)phenyl)-5′,6-dimethyl-4-((1S,2S)- |
| | 2-(2-methyloxazol-4-yl)cyclopropyl)-2H-[1,4′-bipyridin]-2-one |
| 4.168 | 3-chloro-4-((1S,2S)-2-(2,3-dihydropyrazolo[5,1-b]oxazol-7-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.169 | 3-chloro-3′-fluoro-4-((1S,2S)-2-(4-fluoro-1-methyl-1H-pyrazol-3-yl)cyclopropyl)- |
| | 2′-(2-fluoro-3-(methylsulfonyl)phenyl)-5′,6-dimethyl-2H-[1,4′-bipyridin]-2-one |
| 4.170 | 3-chloro-3′-fluoro-2′-(2-fluoro-3-(methylsulfonyl)phenyl)-4-((1S,2S)-2-(isothiazol- |
| | 4-yl)cyclopropyl)-5′,6-dimethyl-2H-[1,4′-bipyridin]-2-one |
| 4.171 | 3-chloro-4-((1S,2S)-2-(6-chloropyrazin-2-yl)cyclopropyl)-3′-fluoro-2′-(2-fluoro-3- |
| | (methylsulfonyl)phenyl)-5′,6-dimethyl-2H-[1,4′-bipyridin]-2-one |
| 4.172 | 3-chloro-4-((1S,2S)-2-(5-chloropyridin-2-yl)cyclopropyl)-3′-fluoro-2′-(2-fluoro-3- |
| | (methylsulfonyl)phenyl)-5′,6-dimethyl-2H-[1,4′-bipyridin]-2-one |
| 4.173 | 3-chloro-3′-fluoro-2′-(2-fluoro-3-((S)-S-methylsulfonimidoyl)phenyl)-5′,6- |
| | dimethy1-4-((1S,2S)-2-(pyridin-2-yl)cyclopropyl)-2H-[1,4′-bipyridin]-2-one |
| 4.174 | 3-chloro-3′-fluoro-2′-(2-fluoro-3-((S)-S-methylsulfonimidoyl)phenyl)-5′,6- |
| | dimethyl-4-((1S,2S)-2-(2-(methyl-d3)-2H-1,2,3-triazol-4-yl)cyclopropyl)-2H-[1,4′- |
| | bipyridin]-2-one |
| 4.175 | 3-chloro-4-((1S,2S)-2-(3,5-difluoropyridin-2-yl)cyclopropyl)-3′-fluoro-2′-(2-fluoro- |
| | 3-((S)-S-methylsulfonimidoyl)phenyl)-5′,6-dimethyl-2H-[1,4′-bipyridin]-2-one |
| 4.176 | 3-chloro-3′-fluoro-2′-(2-fluoro-3-(methylsulfonyl)phenyl)-5′,6-dimethyl-4-((1S,2S)- |
| | 2-(6-(methyl-d3)pyridin-2-yl)cyclopropyl)-2H-[1,4′-bipyridin]-2-one |
| 4.177 | 3-chloro-3′-fluoro-4-((1S,2S)-2-(4-fluoro-1-(methyl-d3)-1H-pyrazol-3- |
| | yl)cyclopropyl)-2′-(2-fluoro-3-(methylsulfonyl)phenyl)-5′,6-dimethyl-2H-[1,4′- |
| | bipyridin]-2-one |
| 4.178 | 3-chloro-3′-fluoro-2′-(2-fluoro-3-(methylsulfonyl)phenyl)-5′,6-dimethyl-4-((1S,2S)- |
| | 2-(1-(methyl-d3)-1H-pyrazol-3-yl)cyclopropyl)-2H-[1,4′-bipyridin]-2-one |
| 4.179 | 3-chloro-3′-fluoro-2′-(2-fluoro-3-(methylsulfonyl)phenyl)-5′,6-dimethyl-4-((1S,2S)- |
| | 2-(1-(methyl-d3)-1H-pyrazol-3-yl)cyclopropyl)-2H-[1,4′-bipyridin]-2-one |
| 4.180 | 3-chloro-3′-fluoro-2′-(2-fluoro-3-(methylsulfonyl)phenyl)-5′,6-dimethyl-4-((1S,2S)- |
| | 2-(1-(methyl-d3)-1H-pyrazol-3-yl)cyclopropyl)-2H-[1,4′-bipyridin]-2-one |
| 4.181 | 3-chloro-4-((1S,2S)-2-(5-chloro-1-methy1-1H-pyrazol-4-yl)cyclopropyl)-3′-fluoro- |
| | 2′-(2-fluoro-3-(methylsulfonyl)phenyl)-5′,6-dimethyl-2H-[1,4′-bipyridin]-2-one |
| 4.182 | 3-chloro-4-((1S,2S)-2-(5-chloro-2-methyl-2H-1,2,3-triazol-4-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.183 | 3-chloro-4-((1S,2S)-2-(1-ethyl-6-oxo-1,6-dihydropyridin-2-yl)cyclopropyl)-3′- |
| | fluoro-2′-(2-fluoro-3-(methylsulfonyl)phenyl)-5′,6-dimethyl-2H-[1,4′-bipyridin]-2- |
| | one |
| 4.184 | 3-chloro-3′-fluoro-2′-(2-fluoro-3-(methylsulfonyl)phenyl)-5′,6-dimethyl-4-((1S,2S)- |
| | 2-(1-(methyl-d3)-6-oxo-1,6-dihydropyridin-2-yl)cyclopropyl)-2H-[1,4′-bipyridin]- |
| | 2-one |
| 4.185 | 3-chloro-2′-(3-(3,5-dimethyl-1H-1,2,4-triazol-1-yl)-2-fluorophenyl)-3′-fluoro-5′,6- |
| | dimethy1-4-((1S,2S)-2-(pyridin-2-yl)cyclopropyl)-2H-[1,4′-bipyridin]-2-one |
| 4.186 | 3-chloro-3′,3″-difluoro-5′,6-dimethyl-2″-(3-methyl-5-(trifluoromethyl)-1H-1,2,4- |
| | triazol-1-yl)-4-((1S,2S)-2-(2-(methyl-d3)-2H-1,2,3-triazol-4-yl)cyclopropyl)-2H- |
| | [1,4′:2′,4″-terpyridin]-2-one |
| 4.187 | 3-chloro-2″-(3,5-dimethyl-1H-1,2,4-triazol-1-yl)-3′,3″-difluoro-4-((1S,2S)-2-(5- |
| | fluoropyrimidin-2-yl)cyclopropyl)-5′,6-dimethy1-2H-[1,4′:2′,4″-terpyridin]-2-one |
| 4.188 | 3-chloro-2′-(3-(3,5-dimethyl-1H-1,2,4-triazol-1-yl)-2-fluorophenyl)-3′-fluoro-5′,6- |
| | dimethyl-4-((1S,2S)-2-(2-(methyl-d3)-2H-1,2,3-triazol-4-yl)cyclopropyl)-2H-[1,4′- |
| | bipyridin]-2-one |
| 4.189 | 3-chloro-2″-(3,5-dimethyl-1H-1,2,4-triazol-1-yl)-3′,3″-difluoro-5′,6-dimethyl-4- |
| | ((1S,2S)-2-(2-(methyl-d3)-2H-1,2,3-triazol-4-yl)cyclopropyl)-2H-[1,4′:2′,4″- |
| | terpyridin]-2-one |
| 4.190 | 3-chloro-4-((1S,2S)-2-(3,5-difluoropyridin-2-yl)cyclopropyl)-3′-fluoro-2′-(2-fluoro- |
| | 3-((4-oxido-1,4λ6-oxathian-4-ylidene)amino)phenyl)-5′,6-dimethyl-2H-[1,4′- |
| | bipyridin]-2-one |
| 4.191 | 3-chloro-3′-fluoro-2′-(2-fluoro-3-((4-oxido-1,4λ6-oxathian-4- |
| | ylidene)amino)phenyl)-5′,6-dimethyl-4-((1S,2S)-2-(2-(methyl-d3)-2H-1,2,3-triazol- |
| | 4-yl)cyclopropyl)-2H-[1,4′-bipyridin]-2-one |
| 4.192 | 3-(3-chloro-3′-fluoro-5′,6-dimethyl-4-((1S,2S)-2-(2-(methyl-d3)-2H-1,2,3-triazol-4- |
| | yl)cyclopropyl)-2-oxo-2H-[1,4′-bipyridin]-2′-yl)-N-cyclopropyl-2-fluorobenzamide |
| 4.193 | 3-chloro-4-((1S,2S)-2-(3,5-difluoropyridin-2-yl)cyclopropyl)-3′-fluoro-2′-(2-fluoro- |
| | 3-(1-methyl-1H-1,2,4-triazol-5-yl)phenyl)-5′,6-dimethyl-2H-[1,4′-bipyridin]-2-one |
| 4.194 | 3-chloro-3′-fluoro-2′-(2-fluoro-3-(1-methyl-1H-1,2,4-triazol-5-yl)phenyl)-4- |
| | ((1S,2S)-2-(5-fluoropyridin-2-yl)cyclopropyl)-5′,6-dimethyl-2H-[1,4′-bipyridin]-2- |
| | one |
| 4.195 | 3-chloro-3′-fluoro-2′-(2-fluoro-3-(1-methyl-1H-1,2,4-triazol-5-yl)phenyl)-5′,6- |
| | dimethy1-4-((1S,2S)-2-(pyridin-2-yl)cyclopropyl)-2H-[1,4′-bipyridin]-2-one |
| 4.196 | 3-chloro-4-((1S,2S)-2-(3,5-difluoropyridin-2-yl)cyclopropyl)-2′-(3-((dimethyl(oxo)-λ⁶- |
| | sulfaneylidene)amino)-2-fluorophenyl)-3′-fluoro-5′,6-dimethyl-2H-[1,4′- |
| | bipyridin]-2-one |
| 4.197 | 3-chloro-3′-fluoro-2′-(2-fluoro-3-(1-methyl-1H-1,2,4-triazol-5-yl)phenyl)-5′,6- |
| | dimethy1-4-((1S,2S)-2-(2-(methyl-d3)-2H-1,2,3-triazol-4-yl)cyclopropyl)-2H-[1,4′- |
| | bipyridin]-2-one |
| 4.198 | 3-chloro-2′-(3-(1-(difluoromethyl)-1H-1,2,4-triazol-5-yl)-2-fluorophenyl)-3′-fluoro- |
| | 5′,6-dimethyl-4-((1S,2S)-2-(2-(methyl-d3)-2H-1,2,3-triazol-4-yl)cyclopropyl)-2H- |
| | [1,4′-bipyridin]-2-one |
| 4.199 | 3-chloro-2″-(3,5-dimethyl-1H-1,2,4-triazol-1-yl)-3′,3″-difluoro-5′,6-dimethyl-4- |
| | ((1S,2S)-2-(1-methyl-1H-pyrazol-4-yl)cyclopropyl)-2H-[1,4′:2′,4″-terpyridin]-2-one |
| 4.200 | 3-chloro-2″-(3,5-dimethyl-1H-1,2,4-triazol-1-yl)-3′,3″-difluoro-5′,6-dimethyl-4- |
| | ((1S,2S)-2-(pyridin-2-yl)cyclopropyl)-2H-[1,4′:2′,4″-terpyridin]-2-one |
| 5.1 | 3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorobenzamide |
| 5.2 | 3-chloro-2′-(2-fluoro-3-(morpholine-4-carbonyl)phenyl)-4-((1S,2S)-2-(4- |
| | fluorophenyl)cyclopropyl)-5′,6-dimethyl-2H-[1,4′-bipyridin]-2-one |
| 5.3 | 3-(3-chloro-4-((1S,2S)-2-(1-(difluoromethyl)-1H-pyrazol-4-yl)cyclopropyl)-5′,6- |
| | dimethy1-2-oxo-2H-[1,4′-bipyridin]-2′-yl)-2-fluoro-N-methylbenzamide |
| 5.4 | 3-chloro-4-((1S,2S)-2-(1-(difluoromethyl)-1H-pyrazol-4-yl)cyclopropyl)-2′-(2- |
| | fluoro-3-(morpholine-4-carbonyl)phenyl)-5′,6-dimethyl-2H-[1,4′-bipyridin]-2-one |
| 5.5 | 2′-(3-((1S,4S)-2-oxa-5-azabicyclo[2.2.1]heptane-5-carbonyl)-2-fluorophenyl)-3- |
| | chloro-4-((1S,2S)-2-(1-(difluoromethyl)-1H-pyrazol-4-yl)cyclopropyl)-5′,6- |
| | dimethy1-2H-[1,4′-bipyridin]-2-one |
| 5.6 | 3-chloro-2′-(3-(3,3-difluoroazetidine-1-carbonyl)-2-fluorophenyl)-4-((1S,2S)-2-(1- |
| | (difluoromethyl)-1H-pyrazol-4-yl)cyclopropyl)-5′,6-dimethy1-2H-[1,4′-bipyridin]-2- |
| | one |
| 5.7 | 3-(3-chloro-4-((1S,2S)-2-(1-(difluoromethyl)-1H-pyrazol-4-yl)cyclopropyl)-5′,6- |
| | dimethyl-2-oxo-2H-[1,4′-bipyridin]-2′-yl)-N-cyclopropyl-2-fluorobenzamide |
| 5.8 | 3-(3-chloro-4-((1S,2S)-2-(1-(difluoromethyl)-1H-pyrazol-4-yl)cyclopropyl)-5′,6- |
| | dimethyl-2-oxo-2H-[1,4′-bipyridin]-2′-yl)-2-fluoro-N-(3- |
| | hydroxybicyclo[1.1.1]pentan-1-yl)benzamide |
| 5.9 | 6-(3-chloro-4-((1S,2S)-2-(4-fluorophenyl)cyclopropyl)-5′,6-dimethyl-2-oxo-2H- |
| | [1,4′-bipyridin]-2′-yl)-5-fluoro-N-methyl-1H-indazole-4-carboxamide |
| 5.10 | 3-chloro-2′-(4-(3,3-difluoroazetidine-1-carbonyl)-5-fluoro-1H-indazol-6-yl)-4- |
| | ((1S,2S)-2-(4-fluorophenyl)cyclopropyl)-5′,6-dimethy1-2H-[1,4′-bipyridin]-2-one |
| 5.11 | 3-chloro-2′-(4-(4,4-difluoropiperidine-1-carbonyl)-5-fluoro-1H-indazol-6-yl)-4- |
| | ((1S,2S)-2-(4-fluorophenyl)cyclopropyl)-5′,6-dimethyl-2H-[1,4′-bipyridin]-2-one |
| 5.12 | 2′-(4-((1S,4S)-2-oxa-5-azabicyclo[2.2.1]heptane-5-carbonyl)-5-fluoro-1H-indazol- |
| | 6-yl)-3-chloro-4-((1S,2S)-2-(4-fluorophenyl)cyclopropyl)-5′,6-dimethyl-2H-[1,4′- |
| | bipyridin]-2-one |
| 5.13 | 3-chloro-2′-(4-((2S,4S)-2,4-dimethylazetidine-1-carbonyl)-5-fluoro-1H-indazol-6- |
| | yl)-4-((1S,2S)-2-(4-fluorophenyl)cyclopropyl)-5′,6-dimethyl-2H-[1,4′-bipyridin]-2- |
| | one |
| 5.14 | 3-chloro-2′-(3-(3,3-difluoroazetidine-1-carbonyl)-2-fluorophenyl)-3′-fluoro-4- |
| | ((1S,2S)-2-(4-fluorophenyl)cyclopropyl)-5′,6-dimethyl-2H-[1,4′-bipyridin]-2-one |
| 5.15 | 3-chloro-3′-fluoro-2′-(2-fluoro-3-(3-hydroxyazetidine-1-carbonyl)phenyl)-4- |
| | ((1S,2S)-2-(4-fluorophenyl)cyclopropyl)-5′,6-dimethyl-2H-[1,4′-bipyridin]-2-one |
| 5.16 | 3-chloro-2′-(3-(3,3-difluoroazetidine-1-carbonyl)-2-fluorophenyl)-4-((1S,2S)-2-(1- |
| | (difluoromethyl)-1H-pyrazol-4-yl)cyclopropyl)-3′-fluoro-5′,6-dimethyl-2H-[1,4′- |
| | bipyridin]-2-one |
| 5.17 | 3-chloro-4-((1S,2S)-2-(1-(difluoromethyl)-1H-pyrazol-4-yl)cyclopropyl)-3′-fluoro- |
| | 2′-(2-fluoro-3-(3-hydroxyazetidine-1-carbonyl)phenyl)-5′,6-dimethyl-2H-[1,4′- |
| | bipyridin]-2-one |
| 5.18 | 3-chloro-3′-fluoro-2′-(2-fluoro-3-(3-(methylsulfonyl)azetidine-1-carbonyl)phenyl)- |
| | 4-((1S,2S)-2-(4-fluorophenyl)cyclopropyl)-5′,6-dimethyl-2H-[1,4′-bipyridin]-2-one |
| 5.19 | 3-chloro-3′-fluoro-2′-(2-fluoro-3-(3-(hydroxymethyl)azetidine-1-carbonyl)phenyl)- |
| | 4-((1S,2S)-2-(4-fluorophenyl)cyclopropyl)-5′,6-dimethyl-2H-[1,4′-bipyridin]-2-one |
| 5.20 | 3-chloro-3′-fluoro-2′-(2-fluoro-3-(3-(2-hydroxypropan-2-yl)azetidine-1- |
| | carbonyl)phenyl)-4-((1S,2S)-2-(4-fluorophenyl)cyclopropyl)-5′,6-dimethyl-2H- |
| | [1,4′-bipyridin]-2-one |
| 5.21 | 1-(3-chloro-4-((1S,2S)-2-(4-fluorophenyl)cyclopropyl)-5′,6-dimethyl-2-oxo-2H- |
| | [1,4′-bipyridin]-2′-yl)-N,N-dimethyl-1H-pyrazole-3-carboxamide |
| 5.22 | 4-(3-chloro-4-((1S,2S)-2-(4-fluorophenyl)cyclopropyl)-5′,6-dimethyl-2-oxo-2H- |
| | [1,4′-bipyridin]-2′-yl)-N,N,1-trimethyl-1H-imidazole-2-carboxamide |
| 5.23 | 4-(3-chloro-4-((1S,2S)-2-(4-fluorophenyl)cyclopropyl)-5′,6-dimethyl-2-oxo-2H- |
| | [1,4′-bipyridin]-2′-yl)-1-methyl-1H-imidazole-2-carboxamide |
| 5.24 | 3-chloro-4-((1S,2S)-2-(5-chloropyridin-3-yl)cyclopropyl)-2′-(3-(3,3- |
| | dimethylpyrrolidine-1-carbonyl)-2-fluorophenyl)-5′,6-dimethyl-2H-[1,4′-bipyridin]- |
| | 2-one |
| 5.25 | 3-chloro-4-((1S,2S)-2-(5-chloropyridin-3-yl)cyclopropyl)-2′-(2-fluoro-3-((S)-3- |
| | hydroxy-3-methylpyrrolidine-1-carbonyl)phenyl)-5′,6-dimethyl-2H-[1,4′-bipyridin]- |
| | 2-one |
| 5.26 | 5-(3-chloro-4-((1S,2S)-2-(4-fluorophenyl)cyclopropyl)-5′,6-dimethyl-2-oxo-2H- |
| | [1,4′-bipyridin]-2′-yl)-N,N,1-trimethyl-1H-imidazole-2-carboxamide |
| 5.27 | 3-chloro-4-((1S,2S)-2-(5-chloropyridin-3-yl)cyclopropyl)-2′-(2-fluoro-3-((R)-3- |
| | hydroxy-3-methylpiperidine-1-carbonyl)phenyl)-5′,6-dimethyl-2H-[1,4′-bipyridin]- |
| | 2-one |
| 5.28 | 3-chloro-4-((1S,2S)-2-(5-chloropyridin-3-yl)cyclopropyl)-2′-(2-fluoro-3-((S)-3- |
| | hydroxy-3-methylpiperidine-1-carbonyl)phenyl)-5′,6-dimethyl-2H-[1,4′-bipyridin]- |
| | 2-one |
| 5.29 | 2′-(3-(8-azabicyclo[3.2.1]octane-8-carbonyl)-2-fluorophenyl)-3-chloro-4-((1S,2S)- |
| | 2-(5-chloropyridin-3-yl)cyclopropyl)-5′,6-dimethyl-2H-[1,4′-bipyridin]-2-one |
| 5.30 | 3-chloro-2′-(3-(3,3-difluoropyrrolidine-1-carbonyl)-2-fluorophenyl)-3′-fluoro-4- |
| | ((1S,2S)-2-(4-fluorophenyl)cyclopropyl)-5′,6-dimethyl-2H-[1,4′-bipyridin]-2-one |
| 5.31 | 3-chloro-3′-fluoro-2′-(2-fluoro-3-((R)-3-hydroxypyrrolidine-1-carbonyl)phenyl)-4- |
| | ((1S,2S)-2-(4-fluorophenyl)cyclopropyl)-5′,6-dimethy1-2H-[1,4′-bipyridin]-2-one |
| 5.32 | 3-chloro-3′-fluoro-2′-(2-fluoro-3-((S)-3-hydroxypyrrolidine-1-carbonyl)phenyl)-4- |
| | ((1S,2S)-2-(4-fluorophenyl)cyclopropyl)-5′,6-dimethyl-2H-[1,4′-bipyridin]-2-one |
| 5.33 | 4-(3-chloro-4-((1S,2S)-2-(5-chloropyridin-3-yl)cyclopropyl)-5′,6-dimethyl-2-oxo- |
| | 2H-[1,4′-bipyridin]-2′-yl)-3-fluoro-N,N-dimethylthiophene-2-carboxamide |
| 5.34 | 3-(3-chloro-4-((1S,2S)-2-(5-fluoropyridin-3-yl)cyclopropyl)-5′,6-dimethyl-2-oxo- |
| | 2H-[1,4′-bipyridin]-2′-yl)-2-fluoro-N-methylbenzamide |
| 5.35 | 1-(3-(3-chloro-3′-fluoro-4-((1S,2S)-2-(4-fluorophenyl)cyclopropyl)-5′,6-dimethy1-2- |
| | oxo-2H-[1,4′-bipyridin]-2′-yl)-2-fluorobenzoyl)azetidine-3-carbonitrile |
| 5.36 | (S)-1-(3-(3-chloro-3′-fluoro-4-((1S,2S)-2-(4-fluorophenyl)cyclopropyl)-5′,6- |
| | dimethy1-2-oxo-2H-[1,4′-bipyridin]-2′-yl)-2-fluorobenzoyl)pyrrolidine-3- |
| | carbonitrile |
| 5.37 | (R)-1-(3-(3-chloro-3′-fluoro-4-((1S,2S)-2-(4-fluorophenyl)cyclopropyl)-5′,6- |
| | dimethyl-2-oxo-2H-[1,4′-bipyridin]-2′-yl)-2-fluorobenzoyl)pyrrolidine-3- |
| | carbonitrile |
| 5.38 | 3-chloro-4-((1S,2S)-2-(5-chloropyridin-3-yl)cyclopropyl)-3′-fluoro-2′-(2-fluoro-3- |
| | (3-hydroxyazetidine-1-carbonyl)phenyl)-5′,6-dimethyl-2H-[1,4′-bipyridin]-2-one |
| 5.39 | 3-chloro-4-((1S,2S)-2-(5-chloropyridin-3-yl)cyclopropyl)-3′-fluoro-2′-(2-fluoro-3- |
| | (3-hydroxy-3-methylazetidine-1-carbonyl)phenyl)-5′,6-dimethyl-2H-[1,4′- |
| | bipyridin]-2-one |
| 5.40 | 3-chloro-4-((1S,2S)-2-(5-chloropyridin-3-yl)cyclopropyl)-3′-fluoro-2′-(2-fluoro-3- |
| | (3-(hydroxymethyl)azetidine-1-carbonyl)phenyl)-5′,6-dimethyl-2H-[1,4′-bipyridin]- |
| | 2-one |
| 5.41 | 3-(3-chloro-4-((1S,2S)-2-(5-fluoropyridin-3-yl)cyclopropyl)-5′,6-dimethyl-2-oxo- |
| | 2H-[1,4′-bipyridin]-2′-yl)-2-fluoro-N,N-bis(methyl-d3)benzamide |
| 5.42 | 3-(3-chloro-4-((1S,2S)-2-(5-chloropyridin-3-yl)cyclopropyl)-5′,6-dimethyl-2-oxo- |
| | 2H-[1,4′-bipyridin]-2′-yl)-2-fluoro-N,N-bis(methyl-d3)benzamide |
| 5.43 | 5-(3-chloro-4-((1S,2S)-2-(5-chloropyridin-3-yl)cyclopropyl)-5′,6-dimethyl-2-oxo- |
| | 2H-[1,4′-bipyridin]-2′-yl)-4-fluoro-N,N-dimethylthiophene-3-carboxamide |
| 5.44 | 3-(3-chloro-4-((1S,2S)-2-(5-chloropyridin-3-yl)cyclopropyl)-3′-fluoro-5′,6- |
| | dimethy1-2-oxo-2H-[1,4′-bipyridin]-2′-yl)-N-cyclopropyl-2-fluorobenzamide |
| 5.45 | 3-(3-chloro-4-((1S,2S)-2-(5-chloropyridin-3-yl)cyclopropyl)-3′-fluoro-5′,6- |
| | dimethyl-2-oxo-2H-[1,4′-bipyridin]-2′-yl)-2-fluoro-N-methylbenzamide |
| 5.46 | N-(1-(3-(3-chloro-3′-fluoro-4-((1S,2S)-2-(4-fluorophenyl)cyclopropyl)-5′,6- |
| | dimethyl-2-oxo-2H-[1,4′-bipyridin]-2′-yl)-2-fluorobenzoyl)azetidin-3- |
| | yl)methanesulfonamide |
| 5.47 | 1-(1-(3-(3-chloro-3′-fluoro-4-((1S,2S)-2-(4-fluorophenyl)cyclopropyl)-5′,6- |
| | dimethy1-2-oxo-2H-[1,4′-bipyridin]-2′-yl)-2-fluorobenzoyl)azetidin-3-yl)-3- |
| | methylurea |
| 5.48 | 3-(3-chloro-4-((1S,2S)-2-(5-fluoropyridin-3-yl)cyclopropyl)-5′,6-dimethyl-2-oxo- |
| | 2H-[1,4′-bipyridin]-2′-yl)-2-fluoro-N-(methyl-d3)benzamide |
| 5.49 | 3-(3-chloro-4-((1S,2S)-2-(5-fluoropyridin-3-yl)cyclopropyl)-5′,6-dimethyl-2-oxo- |
| | 2H-[1,4′-bipyridin]-2′-yl)-N-cyclopropyl-2-fluoro-N-methylbenzamide |
| 5.50 | 3-(3-chloro-3′-fluoro-4-((1S,2S)-2-(5-fluoropyridin-3-yl)cyclopropyl)-5′,6- |
| | dimethyl-2-oxo-2H-[1,4′-bipyridin]-2′-yl)-N-cyclopropyl-2-fluoro-N-(methyl- |
| | d3)benzamide |
| 5.51 | 3-chloro-4-((1S,2S)-2-(5-(difluoromethyl)pyridin-3-yl)cyclopropyl)-3′-fluoro-2′-(2- |
| | fluoro-3-(3-hydroxyazetidine-1-carbonyl)phenyl)-5′,6-dimethyl-2H-[1,4′-bipyridin]- |
| | 2-one |
| 5.52 | 3-(3-chloro-4-((1S,2S)-2-(5-(difluoromethyl)pyridin-3-yl)cyclopropyl)-3′-fluoro- |
| | 5′,6-dimethyl-2-oxo-2H-[1,4′-bipyridin]-2′-yl)-2-fluoro-N-methylbenzamide |
| 5.53 | 3-chloro-3′-fluoro-2′-(2-fluoro-3-(3-(oxetan-3-yl)azetidine-1-carbonyl)phenyl)-4- |
| | ((1S,2S)-2-(4-fluorophenyl)cyclopropyl)-5′,6-dimethyl-2H-[1,4′-bipyridin]-2-one |
| 5.54 | 3-chloro-2′-(3-(3-(difluoromethoxy)azetidine-1-carbonyl)-2-fluorophenyl)-3′- |
| | fluoro-4-((1S,2S)-2-(4-fluorophenyl)cyclopropyl)-5′,6-dimethyl-2H-[1,4′- |
| | bipyridin]-2-one |
| 5.55 | 3-chloro-4-((1S,2S)-2-(5-chloropyridin-3-yl)cyclopropyl)-3′-fluoro-2′-(2-fluoro-3- |
| | ((S)-2-methylazetidine-1-carbonyl)phenyl)-5′,6-dimethy1-2H-[1,4′-bipyridin]-2-one |
| 5.56 | 3-chloro-4-((1S,2S)-2-(5-chloropyridin-3-yl)cyclopropyl)-2′-(3-((2S,4S)-2,4- |
| | dimethylazetidine-1-carbonyl)-2-fluorophenyl)-3′-fluoro-5′,6-dimethyl-2H-[1,4′- |
| | bipyridin]-2-one |
| 5.57 | 3-chloro-4-((1S,2S)-2-(5-chloropyridin-3-yl)cyclopropyl)-3′-fluoro-2′-(2-fluoro-3- |
| | (4-azaspiro[2.3]hexane-4-carbonyl)phenyl)-5′,6-dimethyl-2H-[1,4′-bipyridin]-2-one |
| 5.58 | 3-chloro-4-((1S,2S)-2-(5-chloropyridin-3-yl)cyclopropyl)-3′-fluoro-2′-(2-fluoro-3- |
| | (1,6-diazaspiro[3.3]heptane-1-carbonyl)phenyl)-5′,6-dimethyl-2H-[1,4′-bipyridin]- |
| | 2-one |
| 5.59 | 3-chloro-4-((1S,2S)-2-(5-chloropyridin-3-yl)cyclopropyl)-3′-fluoro-2′-(2-fluoro-3- |
| | ((R)-2-(trifluoromethyl)azetidine-1-carbonyl)phenyl)-5′,6-dimethyl-2H-[1,4′- |
| | bipyridin]-2-one |
| 5.60 | 3-(3-chloro-4-((1S,2S)-2-(5-chloropyridin-3-yl)cyclopropyl)-3′-fluoro-5′,6- |
| | dimethy1-2-oxo-2H-[1,4′-bipyridin]-2′-yl)-2-fluoro-N-(2-hydroxyethyl)benzamide |
| 5.61 | 3-(3-chloro-4-((1S,2S)-2-(5-chloropyridin-3-yl)cyclopropyl)-3′-fluoro-5′,6- |
| | dimethyl-2-oxo-2H-[1,4′-bipyridin]-2′-yl)-2-fluoro-N-(methyl-d3)benzamide |
| 5.62 | 3-(3-chloro-4-((1S,2S)-2-(5-chloropyridin-3-yl)cyclopropyl)-3′-fluoro-5′,6- |
| | dimethyl-2-oxo-2H-[1,4′-bipyridin]-2′-yl)-2-fluoro-N-(3- |
| | hydroxycyclobutyl)benzamide |
| 5.63 | 3-(3-chloro-4-((1S,2S)-2-(5-chloropyridin-3-yl)cyclopropyl)-3′-fluoro-5′,6- |
| | dimethyl-2-oxo-2H-[1,4′-bipyridin]-2′-yl)-2-fluoro-N-(3- |
| | fluorocyclobutyl)benzamide |
| 6.1 | 6-(3-chloro-4-((1S,2S)-2-(4-fluorophenyl)cyclopropyl)-5′,6-dimethyl-2-oxo-2H- |
| | [1,4′-bipyridin]-2′-yl)-7-fluorobenzo[d]oxazol-2(3H)-one |
| 6.2 | 2′-(3-amino-2-fluorophenyl)-3-chloro-4-((1S,2S)-2-(4-fluorophenyl)cyclopropyl)- |
| | 5′,6-dimethyl-2H-[1,4′-bipyridin]-2-one |
| 6.3 | N-(3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorophenyl)acetamide |
| 6.4 | 3-(3-chloro-4-((1S,2S)-2-(1-(difluoromethyl)-1H-pyrazol-4-yl)cyclopropyl)-5′,6- |
| | dimethyl-2-oxo-2H-[1,4′-bipyridin]-2′-yl)-2-fluoro-N,N-dimethylbenzamide |
| 6.5 | N-(3-(3-chloro-4-((1S,2S)-2-(1-(difluoromethyl)-1H-pyrazol-4-yl)cyclopropyl)- |
| | 5′,6-dimethyl-2-oxo-2H-[1,4′-bipyridin]-2′-yl)phenyl)methanesulfonamide |
| 6.6 | 3-chloro-2′-(3-((dimethyl(oxo)-λ⁶-sulfaneylidene)amino)phenyl)-4-((1S,2S)-2-(4- |
| | fluorophenyl)cyclopropyl)-5′,6-dimethyl-2H-[1,4′-bipyridin]-2-one |
| 6.7 | 3-chloro-3″-fluoro-4-((1S,2S)-2-(4-fluorophenyl)cyclopropyl)-2″-(3-hydroxy-3- |
| | methylazetidin-1-yl)-5′,6-dimethyl-2H-[1,4′:2′,4″-terpyridin]-2-one |
| 6.8 | 3-chloro-5″-((dimethyl(oxo)-λ⁶-sulfaneylidene)amino)-4-((1S,2S)-2-(4- |
| | fluorophenyl)cyclopropyl)-5′,6-dimethyl-2H-[1,4′:2′,3″-terpyridin]-2-one |
| 6.9 | 3-chloro-4-((1S,2S)-2-(1-(difluoromethyl)-1H-pyrazol-4-yl)cyclopropyl)-2′-(5- |
| | fluoro-1H-indazol-6-yl)-5′,6-dimethyl-2H-[1,4′-bipyridin]-2-one |
| 6.10 | 3-(3-chloro-4-((1S,2S)-2-(1-(difluoromethyl)-1H-pyrazol-4-yl)cyclopropyl)-5′,6- |
| | dimethyl-2-oxo-2H-[1,4′-bipyridin]-2′-yl)-2-fluorobenzamide |
| 6.11 | 6-(3-chloro-4-((1S,2S)-2-(4-fluorophenyl)cyclopropyl)-5′,6-dimethyl-2-oxo-2H- |
| | [1,4′-bipyridin]-2′-yl)-5-fluoro-3-hydroxy-3-methylindolin-2-one |
| 6.12 | 3-(3-chloro-3′-fluoro-4-((1S,2S)-2-(4-fluorophenyl)cyclopropyl)-5′,6-dimethyl-2- |
| | oxo-2H-[1,4′-bipyridin]-2′-yl)-2-fluorobenzamide |
| 6.13 | 3-(3-chloro-4-((1S,2S)-2-(1-(difluoromethyl)-1H-pyrazol-4-yl)cyclopropyl)-3′- |
| | fluoro-5′,6-dimethyl-2-oxo-2H-[1,4′-bipyridin]-2′-yl)-2-fluorobenzamide |
| 6.14 | 3-(3-chloro-4-((1S,2S)-2-(1-(difluoromethyl)-1H-pyrazol-4-yl)cyclopropyl)-3′- |
| | fluoro-5′,6-dimethyl-2-oxo-2H-[1,4′-bipyridin]-2′-yl)-2-fluoro-N,N- |
| | dimethylbenzamide |
| 6.15 | 3-(3-chloro-3′-fluoro-4-((1S,2S)-2-(4-fluorophenyl)cyclopropyl)-5′,6-dimethyl-2- |
| | oxo-2H-[1,4′-bipyridin]-2′-yl)-2-fluoro-N,N-dimethylbenzamide |
| 6.16 | 6-(3-chloro-4-((1S,2S)-2-(4-fluorophenyl)cyclopropyl)-5′,6-dimethyl-2-oxo-2H- |
| | [1,4′-bipyridin]-2′-yl)-3-hydroxy-3-isopropyl-4-(trifluoromethyl)indolin-2-one |
| 6.17 | (S)-6-(3-chloro-4-((1S,2S)-2-(4-fluorophenyl)cyclopropyl)-5′,6-dimethyl-2-oxo-2H- |
| | [1,4′-bipyridin]-2′-yl)-5-fluoro-3-hydroxy-3-isopropylindolin-2-one |
| 6.18 | (R)-6-(3-chloro-4-((1S,2S)-2-(4-fluorophenyl)cyclopropyl)-5′,6-dimethyl-2-oxo- |
| | 2H-[1,4′-bipyridin]-2′-yl)-5-fluoro-3-hydroxy-3-isopropylindolin-2-one |
| 6.19 | 3-chloro-3″-fluoro-4-((1S,2S)-2-(4-fluorophenyl)cyclopropyl)-5′,6-dimethyl-2-oxo- |
| | 2H-[1,4′:2′,4″-terpyridine]-2″-carbonitrile |
| 6.20 | 3-(3-chloro-3′-fluoro-4-((1S,2S)-2-(4-fluorophenyl)cyclopropyl)-5′,6-dimethyl-2- |
| | oxo-2H-[1,4′-bipyridin]-2′-yl)-2-fluoro-N,N-dimethylbenzamide |
| 6.21 | 5-(3-chloro-4-((1S,2S)-2-(4-fluorophenyl)cyclopropyl)-5′,6-dimethyl-2-oxo-2H- |
| | [1,4′-bipyridin]-2′-yl)-4-fluoro-3,3-dimethylindolin-2-one |
| 6.22 | 4-(3-chloro-4-((1S,2S)-2-(4-fluorophenyl)cyclopropyl)-5′,6-dimethyl-2-oxo-2H- |
| | [1,4′-bipyridin]-2′-yl)-3-fluoro-N,N-dimethylbenzamide |
| 6.23 | 4-(3-chloro-4-((1S,2S)-2-(4-fluorophenyl)cyclopropyl)-5′,6-dimethyl-2-oxo-2H- |
| | [1,4′-bipyridin]-2′-yl)-3-fluorobenzamide |
| 6.24 | 6-(3-chloro-4-((1S,2S)-2-(4-fluorophenyl)cyclopropyl)-5′,6-dimethyl-2-oxo-2H- |
| | [1,4′-bipyridin]-2′-yl)-3-hydroxy-3-methylindolin-2-one |
| 6.25 | 3-chloro-2′-(2-fluoro-3-(methylsulfonyl)phenyl)-4-((1S,2S)-2-(4- |
| | fluorophenyl)cyclopropyl)-5′,6-dimethyl-2H-[1,4′-bipyridin]-2-one |
| 6.26 | 6-(3-chloro-4-((1S,2S)-2-(4-fluorophenyl)cyclopropyl)-5′,6-dimethyl-2-oxo-2H- |
| | [1,4′-bipyridin]-2′-yl)-3,3-difluoroindolin-2-one |
| 6.27 | 3-chloro-2′-(2-fluoro-3-(methylsulfonyl)phenyl)-4-((1S,2S)-2-(5-fluoropyridin-3- |
| | yl)cyclopropyl)-5′,6-dimethyl-2H-[1,4′-bipyridin]-2-one |
| 6.28 | 3-chloro-4-((1S,2S)-2-(5-chloropyridin-3-yl)cyclopropyl)-2′-(2-fluoro-3- |
| | (methylsulfonyl)phenyl)-5′,6-dimethyl-2H-[1,4′-bipyridin]-2-one |
| 6.29 | 3-(3-chloro-4-((1S,2S)-2-(5-fluoropyridin-3-yl)cyclopropyl)-5′,6-dimethyl-2-oxo- |
| | 2H-[1,4′-bipyridin]-2′-yl)-2-fluorobenzamide |
| 6.30 | N-(3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorophenyl)cyclopropanecarboxamide |
| 6.31 | N-(3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorophenyl)-2-hydroxy-2-methylpropanamide |
| 6.32 | N-(3-(3-chloro-4-((1S,2S)-2-(5-chloropyridin-3-yl)cyclopropyl)-5′,6-dimethyl-2- |
| | oxo-2H-[1,4′-bipyridin]-2′-yl)-2-fluorophenyl)-2-hydroxy-2-methylpropanamide |
| 6.33 | 3-chloro-3′-fluoro-2′-(2-fluoro-3-(methylsulfonyl)phenyl)-4-((1S,2S)-2-(5- |
| | fluoropyridin-3-yl)cyclopropyl)-5′,6-dimethyl-2H-[1,4′-bipyridin]-2-one |
| 6.34 | 3-(3-chloro-3′-fluoro-4-((1S,2S)-2-(5-fluoropyridin-3-yl)cyclopropyl)-5′,6- |
| | dimethy1-2-oxo-2H-[1,4′-bipyridin]-2′-yl)-2-fluoro-N-methylbenzamide |
| 6.35 | 3-(3-chloro-3′-fluoro-4-((1S,2S)-2-(5-fluoropyridin-3-yl)cyclopropyl)-5′,6- |
| | dimethy1-2-oxo-2H-[1,4′-bipyridin]-2′-yl)-N-cyclopropyl-2-fluorobenzamide |
| 6.36 | 3-chloro-4-((1S,2S)-2-(5-chloropyridin-3-yl)cyclopropyl)-3′-fluoro-2′-(2-fluoro-3- |
| | (methylsulfonyl)phenyl)-5′,6-dimethyl-2H-[1,4′-bipyridin]-2-one |
| 6.37 | 3-(3-chloro-3′-fluoro-4-((1S,2S)-2-(5-fluoropyridin-3-yl)cyclopropyl)-5′,6- |
| | dimethyl-2-oxo-2H-[1,4′-bipyridin]-2′-yl)-2-fluorobenzamide |
| 6.38 | 3-(3-chloro-4-((1S,2S)-2-(5-chloropyridin-3-yl)cyclopropyl)-3′-fluoro-5′,6- |
| | dimethyl-2-oxo-2H-[1,4′-bipyridin]-2′-yl)-2-fluorobenzamide |
| 6.39 | 3-chloro-4-((1S,2S)-2-(5-chloropyridin-3-yl)cyclopropyl)-3′-fluoro-2′-(2-fluoro-3- |
| | (S-methylsulfonimidoyl)phenyl)-5′,6-dimethyl-2H-[1,4′-bipyridin]-2-one |
| 6.40 | 3-chloro-4-((1S,2S)-2-(5-chloropyridin-3-yl)cyclopropyl)-2′-(2-fluoro-3-(4- |
| | methylthiazol-5-yl)phenyl)-5′,6-dimethyl-2H-[1,4′-bipyridin]-2-one |
| 6.41 | 3-chloro-4-((1S,2S)-2-(5-chloropyridin-3-yl)cyclopropyl)-2′-(2-fluoro-3-(3- |
| | methylisothiazol-4-yl)phenyl)-5′,6-dimethyl-2H-[1,4′-bipyridin]-2-one |
| 6.42 | 3-chloro-4-((1S,2S)-2-(5-chloropyridin-3-yl)cyclopropyl)-2′-(2-fluoro-3-(1-methyl- |
| | 1H-1,2,4-triazol-5-yl)phenyl)-5′,6-dimethyl-2H-[1,4′-bipyridin]-2-one |
| 6.43 | 3-chloro-2′-(3-(2,4-dimethyl-1H-imidazol-1-yl)-2-fluorophenyl)-3′-fluoro-5′,6- |
| | dimethy1-4-((1S,2S)-2-(2-(methyl-d3)-2H-1,2,3-triazol-4-yl)cyclopropyl)-2H-[1,4′- |
| | bipyridin]-2-one |
| 6.44 | 3-chloro-2′-(3-(2,5-dimethyl-2H-1,2,3-triazol-4-yl)-2-fluorophenyl)-3′-fluoro-5′,6- |
| | dimethyl-4-((1S,2S)-2-(2-(methyl-d3)-2H-1,2,3-triazol-4-yl)cyclopropyl)-2H-[1,4′- |
| | bipyridin]-2-one |
| 6.45 | 3-chloro-3′-fluoro-2′-(2-fluoro-3-(2-methyl-1H-imidazol-1-yl)phenyl)-5′,6- |
| | dimethyl-4-((1S,2S)-2-(2-(methyl-d3)-2H-1,2,3-triazol-4-yl)cyclopropyl)-2H-[1,4′- |
| | bipyridin]-2-one |
| 6.46 | 3-chloro-3′-fluoro-2′-(2-fluoro-3-(3-methyl-4H-1,2,4-triazol-4-yl)phenyl)-5′,6- |
| | dimethyl-4-((1S,2S)-2-(2-(methyl-d3)-2H-1,2,3-triazol-4-yl)cyclopropyl)-2H-[1,4′- |
| | bipyridin]-2-one |
| 6.47 | 3-chloro-3′-fluoro-2′-(2-fluoro-3-(3-methyl-4H-1,2,4-triazol-4-yl)phenyl)-4- |
| | ((1S,2S)-2-(5-fluoropyridin-3-yl)cyclopropyl)-5′,6-dimethyl-2H-[1,4′-bipyridin]-2- |
| | one |
| 6.48 | 3-chloro-4-((1S,2S)-2-(1-(difluoromethyl)-1H-pyrazol-4-yl)cyclopropyl)-2′-(3-(3,5- |
| | dimethyl-1H-1,2,4-triazol-1-yl)-2-fluorophenyl)-3′-fluoro-5′,6-dimethyl-2H-[1,4′- |
| | bipyridin]-2-one |
| 6.49 | 3-chloro-3′-fluoro-2′-(2-fluoro-3-((R)-S-methylsulfonimidoyl)phenyl)-4-((1S,2S)-2- |
| | (3-fluoropyridin-2-yl)cyclopropyl)-5′,6-dimethyl-2H-[1,4′-bipyridin]-2-one |
| 6.50 | 3-chloro-3′-fluoro-2′-(2-fluoro-3-((S)-S-methylsulfonimidoyl)phenyl)-4-((1S,2S)-2- |
| | (3-fluoropyridin-2-yl)cyclopropyl)-5′,6-dimethy1-2H-[1,4′-bipyridin]-2-one |
| 6.51 | 3-chloro-4-((1S,2S)-2-(1-(difluoromethyl)-1H-pyrazol-4-yl)cyclopropyl)-3′-fluoro- |
| | 2′-(2-fluoro-3-((R)-S-methylsulfonimidoyl)phenyl)-5′,6-dimethyl-2H-[1,4′- |
| | bipyridin]-2-one |
| 6.52 | 3-chloro-4-((1S,2S)-2-(1-(difluoromethyl)-1H-pyrazol-4-yl)cyclopropyl)-3′-fluoro- |
| | 2′-(2-fluoro-3-((S)-S-methylsulfonimidoyl)phenyl)-5′,6-dimethyl-2H-[1,4′- |
| | bipyridin]-2-one |
| 6.53 | 3-chloro-2′-(2-(3,5-dimethyl-1H-1,2,4-triazol-1-yl)pyrimidin-4-yl)-3′-fluoro-4- |
| | ((1S,2S)-2-(4-fluorophenyl)cyclopropyl)-5′,6-dimethyl-2H-[1,4′-bipyridin]-2-one |
| 6.54 | 3-chloro-2″-(3,5-dimethyl-1H-1,2,4-triazol-1-yl)-3′,3″-difluoro-4-((1S,2S)-2-(4- |
| | fluorophenyl)cyclopropyl)-5′,6-dimethyl-2H-[1,4′:2′,4″-terpyridin]-2-one |
| 6.55 | 3-chloro-2′-(2-(3,5-dimethyl-1H-1,2,4-triazol-1-yl)pyrimidin-4-yl)-3′-fluoro-4- |
| | ((1S,2S)-2-(5-fluoropyridin-3-yl)cyclopropyl)-5′,6-dimethyl-2H-[1,4′-bipyridin]-2- |
| | one |
| 6.56 | 3-chloro-3′-fluoro-2′-(2-fluoro-3-((R)-S-methylsulfonimidoyl)phenyl)-4-((1S,2S)-2- |
| | (4-fluorophenyl)cyclopropyl)-5′,6-dimethyl-2H-[1,4′-bipyridin]-2-one |
| 6.57 | 3-chloro-3′-fluoro-2′-(2-fluoro-3-((S)-S-methylsulfonimidoyl)phenyl)-4-((1S,2S)-2- |
| | (4-fluorophenyl)cyclopropyl)-5′,6-dimethyl-2H-[1,4′-bipyridin]-2-one |
| 6.58 | N′-(3-(3-chloro-3′-fluoro-4-((1S,2S)-2-(5-fluoropyridin-3-yl)cyclopropyl)-5′,6- |
| | dimethyl-2-oxo-2H-[1,4′-bipyridin]-2′-yl)-2-fluorophenyl)-N,N- |
| | dimethylmethanesulfonimidamide |
| 6.59 | 3-chloro-3′-fluoro-2′-(2-fluoro-3-((4-oxido-1,4λ6-oxathian-4- |
| | ylidene)amino)phenyl)-4-((1S,2S)-2-(5-fluoropyridin-3-yl)cyclopropyl)-5′,6- |
| | dimethyl-2H-[1,4′-bipyridin]-2-one |
| 6.60 | 3-chloro-3′-fluoro-2′-(2-fluoro-3-((1-oxidotetrahydro-1λ6-thiophen-1- |
| | ylidene)amino)phenyl)-4-((1S,2S)-2-(5-fluoropyridin-3-yl)cyclopropyl)-5′,6- |
| | dimethyl-2H-[1,4′-bipyridin]-2-one |
| 6.61 | 3-chloro-3′-fluoro-2′-(2-fluoro-3-(1-methyl-1H-1,2,4-triazol-5-yl)phenyl)-4- |
| | ((1S,2S)-2-(3-fluoropyridin-2-yl)cyclopropyl)-5′,6-dimethyl-2H-[1,4′-bipyridin]-2- |
| | one |
| 6.62 | 3-chloro-2″-(3,5-dimethyl-1H-1,2,4-triazol-1-yl)-3′,3″-difluoro-4-((1S,2S)-2-(3- |
| | fluoropyridin-2-yl)cyclopropyl)-5′,6-dimethyl-2H-[1,4′:2′,4″-terpyridin]-2-one |
| 6.63 | 3-chloro-4-((1S,2S)-2-(1-(difluoromethyl)-1H-pyrazol-4-yl)cyclopropyl)-2″-(3,5- |
| | dimethyl-1H-1,2,4-triazol-1-yl)-3′,3″-difluoro-5′,6-dimethyl-2H-[1,4′:2′,4″- |
| | terpyridin]-2-one |
| 6.64 | 3-chloro-3′-fluoro-2′-(2-fluoro-3-(4-methyl-1H-imidazol-1-yl)phenyl)-4-((1S,2S)-2- |
| | (5-fluoropyridin-3-yl)cyclopropyl)-5′,6-dimethyl-2H-[1,4′-bipyridin]-2-one |
| 6.65 | 3-chloro-2″-(3,5-dimethyl-1H-1,2,4-triazol-1-yl)-3′,3″-difluoro-4-((1S,2S)-2-(5- |
| | fluoropyridin-3-yl)cyclopropyl)-5′,6-dimethyl-2H-[1,4′:2′,4″-terpyridin]-2-one |
| 6.66 | 3-chloro-3′,3″-difluoro-4-((1S,2S)-2-(5-fluoropyridin-3-yl)cyclopropyl)-5′,6- |
| | dimethyl-2″-(3-methyl-1H-1,2,4-triazol-1-yl)-2H-[1,4′:2′,4″-terpyridin]-2-one |
| 6.67 | 3-chloro-2′-(3-(1-cyclopropyl-1H-1,2,4-triazol-5-yl)-2-fluorophenyl)-3′-fluoro-4- |
| | ((1S,2S)-2-(5-fluoropyridin-3-yl)cyclopropyl)-5′,6-dimethyl-2H-[1,4′-bipyridin]-2- |
| | one |
| 6.68 | 3-chloro-4-((1S,2S)-2-(5-chloropyridin-3-yl)cyclopropyl)-2′-(2-fluoro-3-(2-methyl- |
| | 2H-tetrazol-5-yl)phenyl)-5′,6-dimethyl-2H-[1,4′-bipyridin]-2-one |
| 6.69 | 3-chloro-4-((1S,2S)-2-(5-chloropyridin-3-yl)cyclopropyl)-2′-(3- |
| | ((difluoromethyl)sulfonyl)-2-fluorophenyl)-3′-fluoro-5′,6-dimethyl-2H-[1,4′- |
| | bipyridin]-2-one |
| 6.70 | 3-chloro-2′-(3-(cyclopropylsulfonyl)-2-fluorophenyl)-3′-fluoro-4-((1S,2S)-2-(5- |
| | fluoropyridin-3-yl)cyclopropyl)-5′,6-dimethyl-2H-[1,4′-bipyridin]-2-one |
| 6.71 | 3-chloro-2′-(3-((difluoromethyl)sulfonyl)-2-fluorophenyl)-3′-fluoro-4-((1S,2S)-2- |
| | (5-fluoropyridin-3-yl)cyclopropyl)-5′,6-dimethyl-2H-[1,4′-bipyridin]-2-one |
| 6.72 | 3-chloro-3′-fluoro-2′-(2-fluoro-3-((2-hydroxy-2-methylpropyl)sulfonyl)phenyl)-4- |
| | ((1S,2S)-2-(5-fluoropyridin-3-yl)cyclopropyl)-5′,6-dimethyl-2H-[1,4′-bipyridin]-2- |
| | one |
| 6.73 | 3-chloro-2′-(2-chloro-3-(methylsulfonyl)phenyl)-3′-fluoro-4-((1S,2S)-2-(5- |
| | fluoropyridin-3-yl)cyclopropyl)-5′,6-dimethyl-2H-[1,4′-bipyridin]-2-one |
| 6.74 | 3-chloro-2′-(4-chloro-2-fluoro-3-(methylsulfonyl)phenyl)-3′-fluoro-4-((1S,2S)-2-(5- |
| | fluoropyridin-3-yl)cyclopropyl)-5′,6-dimethyl-2H-[1,4′-bipyridin]-2-one |
| 6.75 | 3-chloro-3′-fluoro-4-((1S,2S)-2-(5-fluoropyridin-3-yl)cyclopropyl)-5′,6-dimethyl-2′- |
| | (2-methyl-3-(methylsulfonyl)phenyl)-2H-[1,4′-bipyridin]-2-one |
| 6.76 | 3-chloro-2′-(1,1-dioxido-2,3-dihydrobenzo[b]thiophen-4-yl)-3′-fluoro-4-((1S,2S)-2- |
| | (5-fluoropyridin-3-yl)cyclopropyl)-5′,6-dimethyl-2H-[1,4′-bipyridin]-2-one |
| 6.77 | 3-chloro-2″-(3,5-dimethyl-4H-1,2,4-triazol-4-yl)-3′,3″-difluoro-5′,6-dimethyl-4- |
| | ((1S,2S)-2-(2-(methyl-d3)-2H-1,2,3-triazol-4-yl)cyclopropyl)-2H-[1,4′:2′,4″- |
| | terpyridin]-2-one |
| 6.78 | 3-chloro-2′-(3-(3,5-dimethy1-4H-1,2,4-triazol-4-yl)-2,4-difluorophenyl)-3′-fluoro- |
| | 5′,6-dimethyl-4-((1S,2S)-2-(2-(methyl-d3)-2H-1,2,3-triazol-4-yl)cyclopropyl)-2H- |
| | [1,4′-bipyridin]-2-one |
| 6.79 | 3-chloro-2′-(3-(3,5-dimethyl-4H-1,2,4-triazol-4-yl)-2-fluorophenyl)-3′-fluoro-5′,6- |
| | dimethy1-4-((1S,2S)-2-(2-(methyl-d3)-2H-1,2,3-triazol-4-yl)cyclopropyl)-2H-[1,4′- |
| | bipyridin]-2-one |
| 6.80 | 3-chloro-2′-(3-(3,5-dimethyl-1H-1,2,4-triazol-1-yl)-2-fluorophenyl)-3′-fluoro-4- |
| | ((1S,2S)-2-(5-fluoropyrimidin-2-yl)cyclopropyl)-5′,6-dimethy1-2H-[1,4′-bipyridin]- |
| | 2-one |
| 6.81 | 3-chloro-3′-fluoro-2′-(2-fluoro-3-(1-methyl-1H-1,2,4-triazol-5-yl)phenyl)-4- |
| | ((1S,2S)-2-(5-fluoropyrimidin-2-yl)cyclopropyl)-5′,6-dimethyl-2H-[1,4′-bipyridin]- |
| | 2-one |
| 6.82 | 3-chloro-2′-(3-(1,3-dimethyl-1H-pyrazol-5-yl)-2-fluorophenyl)-3′-fluoro-5′,6- |
| | dimethyl-4-((1S,2S)-2-(2-(methyl-d3)-2H-1,2,3-triazol-4-yl)cyclopropyl)-2H-[1,4′- |
| | bipyridin]-2-one |
| 6.83 | 3-chloro-3′-fluoro-2′-(2-fluoro-3-((S)-S-methylsulfonimidoyl)phenyl)-4-((1S,2S)-2- |
| | (5-fluoropyrimidin-2-yl)cyclopropyl)-5′,6-dimethyl-2H-[1,4′-bipyridin]-2-one |
| 6.84 | 3-chloro-2′-(3-(1,3-dimethy1-1H-1,2,4-triazol-5-yl)-2-fluorophenyl)-3′-fluoro-5′,6- |
| | dimethy1-4-((1S,2S)-2-(2-(methyl-d3)-2H-1,2,3-triazol-4-yl)cyclopropyl)-2H-[1,4′- |
| | bipyridin]-2-one |
| 6.85 | 1-(3-(3-chloro-4-((1S,2S)-2-(1-(difluoromethyl)-1H-pyrazol-4-yl)cyclopropyl)-3′- |
| | fluoro-5′,6-dimethyl-2-oxo-2H-[1,4′-bipyridin]-2′-yl)-2-fluorophenyl)-2H-1λ6,2,5- |
| | thiadiazol-4(3H)-one 1-oxide |
| 6.86 | 3-chloro-4-((1S,2S)-2-(1-(difluoromethyl)-1H-pyrazol-4-yl)cyclopropyl)-3′-fluoro- |
| | 2′-(2-fluoro-3-(1-oxido-4,5-dihydro-3H-1λ6-isothiazol-1-yl)phenyl)-5′,6-dimethyl- |
| | 2H-[1,4′-bipyridin]-2-one |
| 6.87 | 3-chloro-3′-fluoro-2′-(7-fluoro-1-methyl-1-oxido-1λ4-benzo[d]isothiazol-6-yl)-4- |
| | ((1S,2S)-2-(5-fluoropyridin-3-yl)cyclopropyl)-5′,6-dimethyl-2H-[1,4′-bipyridin]-2- |
| | one |
| 7.1 | N-(3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorophenyl)-3-methyloxetane-3-carboxamide |
| 7.2 | N-(3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorophenyl)oxetane-3-carboxamide |
| 7.3 | N-(3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorophenyl)-1-(difluoromethyl)cyclopropane-1- |
| | carboxamide |
| 7.4 | N-(3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorophenyl)-1-methoxycyclobutane-1-carboxamide |
| 8.1 | N-(3-(3-chloro-4-((1S,2S)-2-(4-fluorophenyl)cyclopropyl)-5′,6-dimethyl-2-oxo-2H- |
| | [1,4′-bipyridin]-2′-yl)-2-fluorophenyl)-1-fluorocyclopropane-1-carboxamide |
| 8.2 | N-(3-(3-chloro-4-((1S,2S)-2-(5-fluoropyridin-3-yl)cyclopropyl)-5′,6-dimethyl-2- |
| | oxo-2H-[1,4′-bipyridin]-2′-yl)-2-fluorophenyl)-1-methylcyclopropane-1- |
| | carboxamide |
| 9.1 | 3-(3-chloro-4-((1S,2S)-2-(cyclopropylcarbamoyl)cyclopropyl)-5′,6-dimethyl-2-oxo- |
| | 2H-[1,4′-bipyridin]-2′-yl)-2-fluoro-N,N-dimethylbenzamide |
| 9.2 | 3-(3-chloro-5′,6-dimethyl-2-oxo-4-((1S,2S)-2-(phenylcarbamoyl)cyclopropyl)-2H- |
| | [1,4′-bipyridin]-2′-yl)-2-fluoro-N,N-dimethylbenzamide |
| 9.3 | 3-(3-chloro-4-((1S,2S)-2-(1,1-dioxidothiomorpholine-4-carbonyl)cyclopropyl)-5′,6- |
| | dimethyl-2-oxo-2H-[1,4′-bipyridin]-2′-yl)-2-fluoro-N,N-dimethylbenzamide |
| 10.1 | 2″-(3-(aminomethyl)-3-hydroxyazetidin-1-yl)-3-chloro-3″-fluoro-4-((1S,2S)-2-(4- |
| | fluorophenyl)cyclopropyl)-5′,6-dimethyl-2H-[1,4′:2′,4″-terpyridin]-2-one |
| 10.2 | 2″-(3-amino-3-methylazetidin-1-yl)-3-chloro-3″-fluoro-4-((1S,2S)-2-(4- |
| | fluorophenyl)cyclopropyl)-5′,6-dimethyl-2H-[1,4′:2′,4″-terpyridin]-2-one |
| 10.3 | 3-chloro-3″-fluoro-4-((1S,2S)-2-(5-fluoropyridin-3-yl)cyclopropyl)-2″-(3-hydroxy- |
| | 3-methylazetidin-1-yl)-5′,6-dimethyl-2H-[1,4′:2′,4″-terpyridin]-2-one |
| 10.4 | 2″-(3-amino-3-methylazetidin-1-yl)-3-chloro-3″-fluoro-4-((1S,2S)-2-(5- |
| | fluoropyridin-3-yl)cyclopropyl)-5′,6-dimethyl-2H-[1,4′:2′,4″-terpyridin]-2-one |
| 10.5 | 2″-((1-acetyl-4,4-difluoropyrrolidin-3-yl)oxy)-3-chloro-3′,3″-difluoro-4-((1S,2S)-2- |
| | (5-fluoropyridin-3-yl)cyclopropyl)-5′,6-dimethyl-2H-[1,4′:2′,4″-terpyridin]-2-one |
| 10.6 | 2″-((1-acetyl-3-methylpyrrolidin-3-yl)oxy)-3-chloro-3′,3″-difluoro-4-((1S,2S)-2-(5- |
| | fluoropyridin-3-yl)cyclopropyl)-5′,6-dimethyl-2H-[1,4′:2′,4″-terpyridin]-2-one |
| 10.7 | 2″-((1-acetyl-3-methylazetidin-3-yl)oxy)-3-chloro-3′,3″-difluoro-4-((1S,2S)-2-(5- |
| | fluoropyridin-3-yl)cyclopropyl)-5′,6-dimethyl-2H-[1,4′:2′,4″-terpyridin]-2-one |
| 10.8 | 2″-((1-acetylazetidin-3-yl)oxy)-3-chloro-3′,3″-difluoro-4-((1S,2S)-2-(5- |
| | fluoropyridin-3-yl)cyclopropyl)-5′,6-dimethyl-2H-[1,4′:2′,4″-terpyridin]-2-one |
| 10.9 | 3-chloro-3′,3″-difluoro-4-((1S,2S)-2-(5-fluoropyridin-3-yl)cyclopropyl)-2″- |
| | (isopropylamino)-5′,6-dimethyl-2H-[1,4′:2′,4″-terpyridin]-2-one |
| 10.10 | 2″-((1S,4S)-2-oxa-5-azabicyclo[2.2.1]heptan-5-yl)-3-chloro-3′,3″-difluoro-4- |
| | ((1S,2S)-2-(5-fluoropyridin-3-yl)cyclopropyl)-5′,6-dimethyl-2H-[1,4′:2′,4″- |
| | terpyridin]-2-one |
| 10.11 | 2″-((1R,4R)-2-oxa-5-azabicyclo[2.2.1]heptan-5-yl)-3-chloro-3′,3″-difluoro-4- |
| | ((1S,2S)-2-(5-fluoropyridin-3-yl)cyclopropyl)-5′,6-dimethyl-2H-[1,4′:2′,4″- |
| | terpyridin]-2-one |
| 11.1 | N-(3-chloro-3″-fluoro-4-((1S,2S)-2-(4-fluorophenyl)cyclopropyl)-5′,6-dimethyl-2- |
| | oxo-2H-[1,4′:2′,4″-terpyridin]-2″-yl)-1-methylcyclopropane-1-carboxamide |
| 12.1 | 2-fluoro-3-(4-((1S,2S)-2-(4-fluorophenyl)cyclopropyl)-5′,6-dimethyl-2-oxo-2H- |
| | [1,4′-bipyridin]-2′-yl)-N,N-dimethylbenzamide |
| 13.1 | 3-(3-bromo-4-((1S,2S)-2-(4-fluorophenyl)cyclopropyl)-5′,6-dimethyl-2-oxo-2H- |
| | [1,4′-bipyridin]-2′-yl)-2-fluoro-N,N-dimethylbenzamide |
| 14.1 | methyl (3-(3-chloro-4-((1S,2S)-2-(5-chloropyridin-3-yl)cyclopropyl)-5′,6-dimethyl- |
| | 2-oxo-2H-[1,4′-bipyridin]-2′-yl)-2-fluorophenyl)carbamate |
| 14.2 | 3-(3-(3-chloro-4-((1S,2S)-2-(5-chloropyridin-3-yl)cyclopropyl)-5′,6-dimethyl-2- |
| | oxo-2H-[1,4′-bipyridin]-2′-yl)-2-fluorophenyl)-1,1-dimethylurea |
| 14.3 | 3-(3-(3-chloro-4-((1S,2S)-2-(5-chloropyridin-3-yl)cyclopropyl)-5′,6-dimethyl-2- |
| | oxo-2H-[1,4′-bipyridin]-2′-yl)-2-fluorophenyl)oxazolidin-2-one |
| 15.1 | N-(3-(3-chloro-4-((1S,2S)-2-(5-chloropyridin-3-yl)cyclopropyl)-5′,6-dimethyl-2- |
| | oxo-2H-[1,4′-bipyridin]-2′-yl)-2-fluorophenyl)-N,1-dimethylcyclopropane-1- |
| | carboxamide |
| 16.1 | 3-(3-chloro-4-((1S,2S)-2-(3-(cyclopropylcarbamoyl)-5-fluorophenyl)cyclopropyl)- |
| | 5′,6-dimethyl-2-oxo-2H-[1,4′-bipyridin]-2′-yl)-2-fluoro-N,N-dimethylbenzamide |
| 17.1 | 3-chloro-5-((1S,2S)-2-(3-chloro-3′-fluoro-2′-(2-fluoro-3-((R)-2- |
| | (trifluoromethyl)azetidine-1-carbonyl)phenyl)-5′,6-dimethyl-2-oxo-2H-[1,4′- |
| | bipyridin]-4-yl)cyclopropyl)pyridine 1-oxide |
| 17.2 | 3-((1S,2S)-2-(3-chloro-3′-fluoro-2′-(2-fluoro-3-(methylsulfonyl)phenyl)-5′,6- |
| | dimethy1-2-oxo-2H-[1,4′-bipyridin]-4-yl)cyclopropyl)-5-fluoropyridine 1-oxide |
| 17.3 | 3-chloro-5-((1S,2S)-2-(3-chloro-3′-fluoro-2′-(2-fluoro-3-(methylsulfonyl)phenyl)- |
| | 5′,6-dimethyl-2-oxo-2H-[1,4′-bipyridin]-4-yl)cyclopropyl)pyridine 1-oxide |
| 17.4 | 3-((1S,2S)-2-(3-chloro-3′-fluoro-2′-(2-fluoro-3-(methylsulfonyl)phenyl)-5′,6- |
| | dimethy1-2-oxo-2H-[1,4′-bipyridin]-4-yl)cyclopropyl)-5-(difluoromethyl)pyridine |
| | 1-oxide |
| 17.5 | 3-((1S,2S)-2-(3-chloro-3′-fluoro-2′-(2-fluoro-3-(methylsulfonyl)phenyl)-5′,6- |
| | dimethy1-2-oxo-2H-[1,4′-bipyridin]-4-yl)cyclopropyl)-5-(trifluoromethyl)pyridine |
| | 1-oxide |
| 18.1 | 5-chloro-3-(3-fluoro-2-(2-fluoro-3-(methylsulfonyl)phenyl)-5-methylpyridin-4-yl)- |
| | 6-((1S,2S)-2-(5-fluoropyridin-3-yl)cyclopropyl)-2-methylpyrimidin-4(3H)-one |
| 18.2 | 5-chloro-3-(3-fluoro-2-(2-fluoro-3-(methylsulfonyl)phenyl)-5-methylpyridin-4-yl)- |
| | 2-methyl-6-((1S,2S)-2-(2-(methyl-d3)-2H-1,2,3-triazol-4-yl)cyclopropyl)pyrimidin- |
| | 4(3H)-one |
| 18.3 | 5-chloro-3-(2-(3-(3,5-dimethyl-1H-1,2,4-triazol-1-yl)-2-fluorophenyl)-3-fluoro-5- |
| | methylpyridin-4-yl)-2-methyl-6-((1S,2S)-2-(2-(methyl-d3)-2H-1,2,3-triazol-4- |
| | yl)cyclopropyl)pyrimidin-4(3H)-one |
| 18.4 | 5-chloro-3-(2′-(3,5-dimethyl-1H-1,2,4-triazol-1-yl)-3,3′-difluoro-5-methyl-[2,4′- |
| | bipyridin]-4-yl)-2-methyl-6-((1S,2S)-2-(2-(methyl-d3)-2H-1,2,3-triazol-4- |
| | yl)cyclopropyl)pyrimidin-4(3H)-one |
| 18.5 | 5-chloro-6-((1S,2S)-2-(5-fluoro-1-(methyl-d3)-1H-pyrazol-4-yl)cyclopropyl)-3-(3- |
| | fluoro-2-(2-fluoro-3-(methylsulfonyl)phenyl)-5-methylpyridin-4-yl)-2- |
| | methylpyrimidin-4(3H)-one |
| 18.6 | 5-chloro-3-(3-fluoro-2-(2-fluoro-3-(methylsulfonyl)phenyl)-5-methylpyridin-4-yl)- |
| | 2-methyl-6-((1S,2S)-2-(1-(methyl-d3)-1H-pyrazol-4-yl)cyclopropyl)pyrimidin- |
| | 4(3H)-one |
| 18.7 | 5-chloro-3-(3-fluoro-2-(2-fluoro-3-((S)-S-methylsulfonimidoyl)phenyl)-5- |
| | methylpyridin-4-yl)-2-methyl-6-((1S,2S)-2-(2-(methyl-d3)-2H-1,2,3-triazol-4- |
| | yl)cyclopropyl)pyrimidin-4(3H)-one |
| 18.8 | 5-chloro-3-(3-fluoro-2-(2-fluoro-3-(methylsulfonyl)phenyl)-5-methylpyridin-4-yl)- |
| | 6-((1S,2S)-2-(5-fluoropyrimidin-2-yl)cyclopropyl)-2-methylpyrimidin-4(3H)-one |
| 18.9 | 5-chloro-3-(3-fluoro-2-(2-fluoro-3-(methylsulfonyl)phenyl)-5-methylpyridin-4-yl)- |
| | 2-methyl-6-((1S,2S)-2-(4-(methyl-d3)pyrimidin-2-yl)cyclopropyl)pyrimidin-4(3H)- |
| | one |
| 19.1 | 5-chloro-3-(6′-(3,5-dimethyl-1H-1,2,4-triazol-1-yl)-3,5′-difluoro-5-methyl-[2,2′- |
| | bipyridin]-4-yl)-2-methyl-6-((1S,2S)-2-(2-(methyl-d3)-2H-1,2,3-triazol-4- |
| | yl)cyclopropyl)pyrimidin-4(3H)-one |
| 19.2 | 3-chloro-6″-(3,5-dimethyl-1H-1,2,4-triazol-1-yl)-3′,5″-difluoro-4-((1S,2S)-2-(5- |
| | fluoropyridin-3-yl)cyclopropyl)-5′,6-dimethyl-2H-[1,4′:2′,2″-terpyridin]-2-one |
| 19.3 | 3-chloro-4-((1S,2S)-2-(1-(difluoromethyl)-1H-pyrazol-4-yl)cyclopropyl)-6″-(3,5- |
| | dimethyl-1H-1,2,4-triazol-1-yl)-3′,5″-difluoro-5′,6-dimethyl-2H-[1,4′:2′,2″- |
| | terpyridin]-2-one |
| 19.4 | 3-chloro-4-((1S,2S)-2-(1-(difluoromethyl)-1H-pyrazol-4-yl)cyclopropyl)-6″-(3,5- |
| | dimethyl-1H-1,2,4-triazol-1-yl)-3′-fluoro-5′,6-dimethyl-2H-[1,4′:2′,2″-terpyridin]-2- |
| | one |
| 19.5 | 3-chloro-6″-(3,5-dimethyl-1H-1,2,4-triazol-1-yl)-3′-fluoro-5′,6-dimethyl-4-((1S,2S)- |
| | 2-(2-(methyl-d3)-2H-1,2,3-triazol-4-yl)cyclopropyl)-2H-[1,4′:2′,2″-terpyridin]-2- |
| | one |
| 19.6 | 3-chloro-6″-(3,5-dimethyl-1H-1,2,4-triazol-1-yl)-3′,5″-difluoro-5′,6-dimethyl-4- |
| | ((1S,2S)-2-(2-(methyl-d3)-2H-1,2,3-triazol-4-yl)cyclopropyl)-2H-[1,4′:2′,2″- |
| | terpyridin]-2-one |
| 20.1 | 3-chloro-4-((1S,2S)-2-(1-(difluoromethyl)-1H-pyrazol-4-yl)cyclopropyl)-2′-(3-((R)- |
| | N,S-dimethylsulfonimidoyl)-2-fluorophenyl)-3′-fluoro-5′,6-dimethyl-2H-[1,4′- |
| | bipyridin]-2-one |
| 20.2 | 3-chloro-4-((1S,2S)-2-(1-(difluoromethyl)-1H-pyrazol-4-yl)cyclopropyl)-2′-(3-((S)- |
| | N,S-dimethylsulfonimidoyl)-2-fluorophenyl)-3′-fluoro-5′,6-dimethyl-2H-[1,4′- |
| | bipyridin]-2-one |
| 20.3 | 3-chloro-2′-(3-((R)-N-cyclopropyl-S-methylsulfonimidoyl)-2-fluorophenyl)-4- |
| | ((1S,2S)-2-(1-(difluoromethyl)-1H-pyrazol-4-yl)cyclopropyl)-3′-fluoro-5′,6- |
| | dimethy1-2H-[1,4′-bipyridin]-2-one |
| 20.4 | 3-chloro-2′-(3-((S)-N-cyclopropyl-S-methylsulfonimidoyl)-2-fluorophenyl)-4- |
| | ((1S,2S)-2-(1-(difluoromethyl)-1H-pyrazol-4-yl)cyclopropyl)-3′-fluoro-5′,6- |
| | dimethyl-2H-[1,4′-bipyridin]-2-one |
| 21.1 | N-(2-(4-(3-chloro-3′-fluoro-4-((1S,2S)-2-(5-fluoropyridin-3-yl)cyclopropyl)-5′,6- |
| | dimethyl-2-oxo-2H-[1,4′-bipyridin]-2′-yl)pyrimidin-2-yl)propan-2-yl)acetamide |
| 22.1 | 3-chloro-3′-fluoro-4-((1S,2S)-2-(5-fluoropyridin-3-yl)cyclopropyl)-2′-(3-(2- |
| | hydroxypropan-2-yl)-1H-pyrazol-1-yl)-5′,6-dimethyl-2H-[1,4′-bipyridin]-2-one |

Compounds prepared in the Final Procedures were characterized by ES/MS and/or 1H NMR. Data are provided in Table 16.

TABLE 16
ES/MS and 1H NMR data for compounds prepared in Final Procedures

| Example | ES/MS m/z | |
|---|---|---|
| Number | (M + H)⁺ | 1H NMR |
| 1.1 | 501.1 | 1H NMR (400 MHz, DMSO-d6) δ 8.96 (dd, J = 5.2, 1.5 Hz, 1H), 8.90- |
| | | 8.81 (m, 1H), 8.67 (d, J = 10.1 Hz, 1H), 8.23 (dd, J = 5.2, 1.8 Hz, |
| | | 1H), 7.40-7.27 (m, 4H), 7.27-7.16 (m, 1H), 6.01 (d, J = 9.6 Hz, 1H), |
| | | 2.96-2.63 (m, 2H), 2.38-2.22 (m, 1H), 2.12-2.03 (m, 3H), 1.92- |
| | | 1.80 (m, 3H), 1.71-1.47 (m, 7H), 1.28-1.12 (m, 2H). |
| 1.2 | 517.1 | 1H NMR (400 MHz, DMSO-d6) δ 8.97 (d, J = 5.2 Hz, 1H), 8.86 (s, |
| | | 1H), 8.69 (s, 1H), 8.24 (d, J = 5.2 Hz, 1H), 7.28-7.19 (m, 1H), 6.88- |
| | | 6.74 (m, 3H), 6.23 (d, J = 3.5 Hz, 1H), 3.76 (s, 3H), 2.47-2.40 (m, |
| | | 1H), 2.11 (s, 3H), 1.93 (s, 3H), 1.80-1.62 (m, 2H), 1.54 (s, 3H), 1.52 |
| | | (s, 3H). |
| 1.3 | 505.1 | 1H NMR (400 MHz, DMSO-d6) δ 8.97 (d, J = 5.2 Hz, 1H), 8.86 (s, |
| | | 1H), 8.69 (s, 1H), 8.24 (d, J = 5.1 Hz, 1H), 7.32 (ddd, J = 8.8, 6.8, 5.4 |
| | | Hz, 2H), 7.15 (td, J = 8.8, 1.8 Hz, 2H), 6.23 (d, J = 3.6 Hz, 1H), 2.59- |
| | | 2.52 (m, 1H), 2.11 (s, 3H), 1.93 (s, 3H), 1.77-1.64 (m, 2H), 1.53 (s, |
| | | 3H), 1.52 (s, 3H). |
| 1.4 | 505.1 | 1H NMR (400 MHz, DMSO-d6) δ 8.97 (d, J = 5.1 Hz, 1H), 8.86 (s, |
| | | 1H), 8.69 (s, 1H), 8.24 (d, J = 5.1 Hz, 1H), 7.39-7.26 (m, 2H), 7.21- |
| | | 7.09 (m, 2H), 6.27-6.19 (m, 1H), 2.59-2.53 (m, 1H), 2.11 (s, 3H), |
| | | 1.93 (s, 3H), 1.74-1.62 (m, 2H), 1.54 (s, 3H), 1.52 (s, 3H). |
| 1.5 | 505.1 | 1H NMR (400 MHz, DMSO-d6) δ 8.97 (d, J = 5.1 Hz, 1H), 8.86 (s, |
| | | 1H), 8.69 (s, 1H), 8.24 (d, J = 5.1 Hz, 1H), 7.31 (dt, J = 8.4, 6.1 Hz, |
| | | 2H), 7.21-7.09 (m, 2H), 6.27-6.20 (m, 1H), 2.11 (s, 3H), 1.93 (s, |
| | | 3H), 1.78-1.64 (m, 2H), 1.53 (s, 3H), 1.52 (s, 3H). |
| 1.6 | 522.9 | 1H NMR (400 MHz, DMSO-d6) δ 8.97 (d, J = 5.1 Hz, 1H), 8.86 (s, |
| | | 1H), 8.69 (s, 0.5H), 8.67 (s, 0.5H), 8.24 (d, J = 5.1 Hz, 1H), 7.45-7.30 |
| | | (m, 2H), 7.21-7.11 (m, 1H), 6.23 (d, J = 3.8 Hz, 1H), 2.60-2.52 (m, |
| | | 2H), 2.11 (s, 3H), 1.93 (s, 3H), 1.81-1.66 (m, 2H), 1.53 (s, 3H), 1.52 |
| | | (s, 3H). |
| 1.7 | 523.1 | 1H NMR (400 MHz, DMSO-d6) δ 8.97 (d, J = 5.2 Hz, 1H), 8.86 (s, |
| | | 1H), 8.75 (s, 0.5H), 8.69 (s, 0.5H), 8.24 (d, J = 5.2 Hz, 1H), 7.34 (q, J = |
| | | 7.7 Hz, 1H), 7.25 (td, J = 10.1, 9.3, 2.7 Hz, 1H), 7.08 (t, J = 8.6 Hz, |
| | | 1H), 6.28 (d, J = 2.3 Hz, 1H), 2.62-2.52 (m, 2H), 2.12 (s, 1.5H), 2.10 |
| | | (s, 1.5H), 1.93 (s, 3H), 1.83-1.64 (m, 2H), 1.53 (s, 3H), 1.52 (s, 3H). |
| 1.9 | 539.1 | 1H NMR (400 MHz, DMSO-d6) δ 8.98 (d, J = 5.2 Hz, 1H), 8.87 (s, |
| | | 1H), 8.69 (d, J = 6.7 Hz, 1H), 8.25 (d, J = 5.1 Hz, 1H), 7.52 (td, J = 8.1, |
| | | 1.7 Hz, 1H), 7.43-7.29 (m, 1H), 7.24-7.10 (m, 1H), 6.24 (d, J = 4.1 |
| | | Hz, 1H), 2.60-2.52 (m, 2H), 2.11 (d, J = 1.6 Hz, 3H), 1.94 (d, J = 1.7 |
| | | Hz, 3H), 1.85-1.67 (m, 2H), 1.54 (s, 3H), 1.53 (s, 3H). |
| 1.10 | 523.2 | 1H NMR (400 MHz, DMSO-d6) δ 8.98 (d, J = 5.1 Hz, 1H), 8.87 (d, J = |
| | | 1.5 Hz, 1H), 8.74 (d, J = 29.2 Hz, 1H), 8.25 (d, J = 5.1 Hz, 1H), 7.43- |
| | | 7.29 (m, 1H), 7.19-7.00 (m, 2H), 6.27 (d, J = 3.7 Hz, 1H), 2.86-2.72 |
| | | (m, 1H), 2.48-2.38 (m, 1H), 2.12 (d, J = 9.5 Hz, 3H), 1.94 (s, 3H), |
| | | 1.85-1.65 (m, 2H), 1.54 (d, J = 1.6 Hz, 3H), 1.53 (s, 3H). |
| 1.11 | 539.0 | 1H NMR (400 MHz, DMSO-d6) δ 8.97 (d, J = 5.1 Hz, 1H), 8.87 (s, |
| | | 1H), 8.68 (d, J = 9.7 Hz, 1H), 8.24 (d, J = 5.2 Hz, 1H), 7.53 (ddd, J = |
| | | 12.3, 7.0, 2.1 Hz, 1H), 7.42-7.24 (m, 2H), 6.23 (d, J = 3.2 Hz, 1H), |
| | | 2.11 (s, 3H), 1.93 (s, 3H), 1.80-1.64 (m, 2H), 1.58-1.44 (m, 6H), |
| | | 1.23-1.12 (m, 2H). |
| 1.12 | 521.2 | 1H NMR (400 MHz, DMSO-d6) δ 8.98 (d, J = 5.1 Hz, 1H), 8.87 (s, |
| | | 1H), 8.73 (d, J = 25.4 Hz, 1H), 8.25 (d, J = 5.1 Hz, 1H), 7.49 (dd, J = |
| | | 7.7, 1.3 Hz, 1H), 7.42-7.19 (m, 4H), 6.31 (s, 1H), 2.77-2.58 (m, 1H), |
| | | 2.50-2.42 (m, 1H), 2.11 (d, J = 8.5 Hz, 3H), 1.95 (s, 3H), 1.88-1.63 |
| | | (m, 2H), 1.54 (s, 3H), 1.53 (s, 3H). |
| 1.13 | 527.2 | 1H NMR (400 MHz, DMSO-d6) δ 8.98 (d, J = 5.1 Hz, 1H), 8.87 (s, |
| | | 1H), 8.70 (d, J = 5.1 Hz, 1H), 8.25 (d, J = 5.1 Hz, 1H), 7.14 (dd, J = 8.3, |
| | | 6.4 Hz, 2H), 7.03 (dd, J = 8.1, 1.5 Hz, 2H), 6.22 (d, J = 3.7 Hz, 1H), |
| | | 2.51-2.37 (m, 2H), 2.12 (s, 3H), 1.93 (d, J = 1.7 Hz, 3H), 1.93-1.87 |
| | | (m, 2H), 1.77-1.62 (m, 2H), 1.54 (s, 3H), 1.53 (s, 3H), 1.00-0.89 (m, |
| | | 2H), 0.73-0.55 (m, 2H). |
| 1.14 | 554.2 | 1H NMR (400 MHz, DMSO-d6) δ 8.98 (d, J = 5.1 Hz, 1H), 8.87 (s, |
| | | 1H), 8.70 (s, 1H), 8.43 (d, J = 2.7 Hz, 1H), 8.25 (d, J = 5.2 Hz, 1H), |
| | | 7.69-7.51 (m, 2H), 7.29 (t, J = 73.6 Hz, 1H), 6.27 (d, J = 4.0 Hz, 1H), |
| | | 2.87-2.75 (m, 2H), 2.12 (s, 3H), 1.94 (s, 3H), 1.85-1.75 (m, 1H), |
| | | 1.75-1.65 (m, 1H), 1.54 (s, 3H), 1.53 (s, 3H). |
| 1.15 | 541.1 | 1H NMR (400 MHz, DMSO-d6) δ 9.04-8.92 (m, 1H), 8.87 (s, 1H), |
| | | 8.72 (d, J = 22.2 Hz, 1H), 8.24 (dd, J = 5.2, 0.7 Hz, 1H), 7.37-7.25 (m, |
| | | 1H), 7.25-7.09 (m, 1H), 6.34-6.24 (m, 1H), 2.71-2.53 (m, 1H), |
| | | 2.11 (d, J = 5.0 Hz, 3H), 1.94 (s, 3H), 1.86-1.64 (m, 2H), 1.60-1.44 |
| | | (m, 6H), 1.16-0.93 (m, 1H). |
| 1.16 | 527.1 | 1H NMR (400 MHz, DMSO-d6) δ 8.98 (d, J = 5.2 Hz, 1H), 8.87 (s, |
| | | 1H), 8.69 (d, J = 3.5 Hz, 1H), 8.25 (d, J = 5.1 Hz, 1H), 8.20 (d, J = 4.0 |
| | | Hz, 1H), 7.81 (d, J = 5.8 Hz, 1H), 7.76 (t, J = 59.3 Hz, 1H), 6.19 (d, J = |
| | | 3.8 Hz, 1H), 2.47-2.39 (m, 1H), 2.37-2.26 (m, 1H), 2.11 (s, 3H), |
| | | 1.93 (s, 3H), 1.72-1.59 (m, 2H), 1.54 (s, 3H), 1.53 (s, 3H). |
| 1.17 | 527.1 | 1H NMR (400 MHz, DMSO-d6) δ 8.98 (d, J = 5.1 Hz, 1H), 8.87 (s, |
| | | 1H), 8.69 (d, J = 3.5 Hz, 1H), 8.25 (d, J = 5.2 Hz, 1H), 8.20 (d, J = 4.0 |
| | | Hz, 1H), 7.81 (d, J = 5.7 Hz, 1H), 7.76 (t, J = 59.3 Hz, 1H), 6.19 (d, J = |
| | | 3.8 Hz, 1H), 2.48-2.39 (m, 1H), 2.39-2.26 (m, 1H), 2.11 (s, 3H), |
| | | 1.93 (s, 3H), 1.75-1.60 (m, 2H), 1.54 (s, 3H), 1.53 (s, 3H). |
| 1.18 | 528.5 | 1H NMR (400 MHz, DMSO-d6) δ 8.97 (dd, J = 5.1, 0.7 Hz, 1H), 8.86 |
| | | (d, J = 2.0 Hz, 1H), 8.69 (d, J = 10.8 Hz, 1H), 8.24 (d, J = 5.2 Hz, 1H), |
| | | 7.56 (s, 1H), 7.25-7.15 (m, 1H), 7.12 (d, J = 7.7 Hz, 1H), 6.23 (d, J = |
| | | 2.7 Hz, 1H), 2.81 (tt, J = 9.7, 5.5 Hz, 1H), 2.66-2.55 (m, 1H), 2.16- |
| | | 2.02 (m, 4H), 1.93 (s, 3H), 1.83-1.58 (m, 2H), 1.58-1.45 (m, 6H), |
| | | 1.01-0.86 (m, 4H). |
| 1.19 | 508.4 | 1H NMR (400 MHz, DMSO-d6) δ 9.01-8.94 (m, 1H), 8.86 (s, 1H), |
| | | 8.73-8.66 (m, 1H), 8.24 (d, J = 5.1 Hz, 1H), 7.29 (d, J = 4.4 Hz, 1H), |
| | | 6.23-6.17 (m, 1H), 2.74-2.57 (m, 5H), 2.14-2.07 (m, 3H), 1.97- |
| | | 1.87 (m, 3H), 1.80-1.58 (m, 2H), 1.58-1.45 (m, 6H). |
| 1.20 | 530.4 | 1H NMR (400 MHz, DMSO-d6) δ 8.97 (d, J = 5.2 Hz, 1H), 8.87 (s, |
| | | 1H), 8.72-8.62 (m, 1H), 8.27-8.19 (m, 1H), 7.91-7.82 (m, 1H), |
| | | 7.50 (dd, J = 10.9, 8.2 Hz, 1H), 7.41-7.32 (m, 1H), 6.30-6.23 (m, |
| | | 1H), 2.67-2.55 (m, 2H), 2.16-2.05 (m, 3H), 1.98-1.79 (m, 5H), |
| | | 1.59-1.45 (m, 6H). |
| 1.21 | 512.4 | 1H NMR (400 MHz, DMSO-d6) δ 8.97 (d, J = 5.1 Hz, 1H), 8.90-8.84 |
| | | (m, 1H), 8.72-8.63 (m, 1H), 8.24 (d, J = 5.1 Hz, 1H), 7.79 (dd, J = 8.4, |
| | | 1.8 Hz, 2H), 7.54-7.43 (m, 2H), 6.31-6.23 (m, 1H), 2.66-2.52 (m, |
| | | 2H), 2.15-2.06 (m, 3H), 1.98-1.89 (m, 3H), 1.90-1.75 (m, 2H), |
| | | 1.58-1.48 (m, 6H). |
| 1.22 | 555.9 | 1H NMR (400 MHz, DMSO-d6) δ 8.98 (d, J = 5.1 Hz, 1H), 8.95-8.89 |
| | | (m, 1H), 8.87 (s, 1H), 8.69 (d, J = 9.8 Hz, 1H), 8.25 (d, J = 5.1 Hz, 1H), |
| | | 8.14 (dd, J = 8.4, 2.4 Hz, 1H), 7.80-7.70 (m, 1H), 6.31 (d, J = 4.6 Hz, |
| | | 1H), 2.98-2.77 (m, 2H), 2.14-2.07 (m, 3H), 1.95 (d, J = 1.6 Hz, 3H), |
| | | 1.93-1.77 (m, 2H), 1.58-1.49 (m, 6H). |
| 1.23 | 555.9 | 1H NMR (400 MHz, DMSO-d6) δ 8.98 (d, J = 5.1 Hz, 1H), 8.88 (s, |
| | | 1H), 8.78 (dd, J = 6.9, 2.1 Hz, 1H), 8.70 (d, J = 6.4 Hz, 1H), 8.25 (dd, J = |
| | | 5.1, 0.9 Hz, 1H), 7.97 (ddd, J = 8.1, 5.7, 2.2 Hz, 1H), 7.87 (dd, J = |
| | | 8.2, 3.0 Hz, 1H), 6.31 (d, J = 4.5 Hz, 1H), 2.77-2.59 (m, 2H), 2.17- |
| | | 2.09 (m, 3H), 1.95 (s, 3H), 1.93-1.78 (m, 2H), 1.60-1.47 (m, 6H). |
| 1.24 | 571.9 | 1H NMR (400 MHz, DMSO-d6) δ 8.98 (d, J = 5.1 Hz, 1H), 8.93-8.85 |
| | | (m, 1H), 8.69 (d, J = 9.7 Hz, 1H), 8.65-8.58 (m, 1H), 8.25 (d, J = 5.1 |
| | | Hz, 1H), 7.85 (ddd, J = 8.6, 2.8, 1.2 Hz, 1H), 7.71-7.59 (m, 1H), 6.28 |
| | | (d, J = 4.1 Hz, 1H), 2.91-2.73 (m, 2H), 2.15-2.09 (m, 3H), 1.94 (d, J = |
| | | 1.5 Hz, 3H), 1.87-1.68 (m, 2H), 1.59-1.48 (m, 6H). |
| 1.25 | 571.9 | 1H NMR (400 MHz, DMSO-d6) δ 8.98 (d, J = 5.1 Hz, 1H), 8.87 (s, |
| | | 1H), 8.70 (d, J = 5.3 Hz, 1H), 8.38 (dd, J = 6.3, 2.5 Hz, 1H), 8.25 (dd, J = |
| | | 5.1, 0.8 Hz, 1H), 7.92 (ddd, J = 8.9, 6.6, 2.6 Hz, 1H), 7.28 (dd, J = |
| | | 8.5, 2.8 Hz, 1H), 6.32-6.23 (m, 1H), 2.71-2.54 (m, 2H), 2.16-2.08 |
| | | (m, 3H), 1.99-1.91 (m, 3H), 1.87-1.71 (m, 2H), 1.58-1.47 (m, 6H). |
| 1.26 | 562.1 | 1H NMR (400 MHz, DMSO-d6) δ 8.98 (d, J = 5.1 Hz, 1H), 8.88 (s, |
| | | 1H), 8.69 (d, J = 9.1 Hz, 1H), 8.25 (d, J = 5.2 Hz, 1H), 8.11-8.05 (m, |
| | | 1H), 6.29 (d, 1H), 3.00-2.80 (m, 1H), 2.72-2.60 (m, 1H), 2.12 (s, |
| | | 3H), 1.94 (t, J = 1.0 Hz, 3H), 1.90-1.82 (m, 2H), 1.54 (s, 3H), 1.53 (s, 3H). |
| 1.27 | 562.1 | 1H NMR (400 MHz, DMSO-d6) δ 8.98 (d, J = 5.2 Hz, 1H), 8.88 (s, |
| | | 1H), 8.69 (d, J = 9.1 Hz, 1H), 8.25 (d, J = 5.2 Hz, 1H), 8.11-8.02 (m, |
| | | 1H), 6.29 (d, 1H), 3.00-2.81 (m, 1H), 2.72-2.62 (m, 1H), 2.12 (s, |
| | | 3H), 1.94 (s, 3H), 1.90-1.79 (m, 2H), 1.54 (s, 3H), 1.53 (s, 3H). |
| 1.28 | 539.1 | 1H NMR (400 MHz, DMSO-d6) δ 8.98 (dd, J = 5.2, 0.9 Hz, 1H), 8.87 |
| | | (s, 1H), 8.72 (d, J = 22.4 Hz, 1H), 8.25 (d, J = 5.1 Hz, 1H), 7.44 (dd, J = |
| | | 10.3, 2.0 Hz, 1H), 7.36-7.26 (m, 2H), 6.29 (d, J = 2.1 Hz, 1H), 2.63- |
| | | 2.53 (m, 2H), 2.12 (d, J = 4.6 Hz, 3H), 1.94 (s, 3H), 1.84-1.62 (m, |
| | | 2H), 1.54 (d, J = 1.4 Hz, 3H), 1.53 (d, J = 2.0 Hz, 3H). |
| 1.29 | 571.1 | 1H NMR (400 MHz, DMSO-d6) δ 8.98 (d, J = 5.1 Hz, 1H), 8.87 (s, |
| | | 1H), 8.69 (d, J = 7.6 Hz, 1H), 8.25 (d, J = 5.2 Hz, 1H), 7.47 (td, J = 8.1, |
| | | 1.5 Hz, 1H), 7.38-7.28 (m, 2H), 7.26-7.17 (m, 1H), 6.25 (d, J = 3.9 |
| | | Hz, 1H), 2.70-2.53 (m, 2H), 2.12 (s, 3H), 1.94 (d, J = 1.6 Hz, 3H), |
| | | 1.84-1.67 (m, 2H), 1.54 (s, 3H), 1.53 (d, J = 1.9 Hz, 3H). |
| 1.30 | 555.2 | 1H NMR (400 MHz, DMSO-d6) δ 8.98 (d, J = 5.1 Hz, 1H), 8.87 (s, |
| | | 1H), 8.70 (d, J = 1.6 Hz, 1H), 8.25 (d, J = 5.1 Hz, 1H), 7.73-7.65 (m, |
| | | 2H), 7.51 (t, J = 7.8 Hz, 2H), 6.28 (d, J = 4.3 Hz, 1H), 2.72-2.54 (m, |
| | | 2H), 2.12 (s, 3H), 1.95 (d, J = 1.9 Hz, 3H), 1.88-1.73 (m, 2H), 1.54 (d, |
| | | J = 1.5 Hz, 3H), 1.53 (d, J = 2.5 Hz, 3H). |
| 1.31 | 513.0 | 1H NMR (400 MHz, DMSO-d6) δ 8.98 (d, J = 5.2 Hz, 1H), 8.88 (s, |
| | | 1H), 8.73-8.62 (m, 2H), 8.25 (d, J = 5.1 Hz, 1H), 8.02 (dd, J = 6.5, 1.7 |
| | | Hz, 1H), 7.70 (td, J = 6.2, 5.8, 1.9 Hz, 1H), 6.33-6.26 (m, 1H), 2.79- |
| | | 2.58 (m, 2H), 2.16-2.09 (m, 3H), 1.95 (s, 3H), 1.93-1.83 (m, 2H), |
| | | 1.60-1.49 (m, 6H). |
| 1.32 | 512.9 | 1H NMR (400 MHz, DMSO-d6) δ 8.98 (d, J = 5.1 Hz, 1H), 8.95-8.86 |
| | | (m, 3H), 8.69 (d, J = 10.9 Hz, 1H), 8.30-8.20 (m, 2H), 6.35-6.26 (m, |
| | | 1H), 2.72-2.60 (m, 2H), 2.16-2.09 (m, 3H), 1.95 (s, 3H), 1.92-1.77 |
| | | (m, 2H), 1.60-1.48 (m, 6H). |
| 1.33 | 555.9 | 1H NMR (400 MHz, DMSO-d6) δ 8.98 (d, J = 5.2 Hz, 1H), 8.94-8.80 |
| | | (m, 3H), 8.69 (d, J = 15.5 Hz, 1H), 8.25 (d, J = 5.1 Hz, 1H), 8.12 (dt, J = |
| | | 11.9, 2.3 Hz, 1H), 6.35-6.26 (m, 1H), 2.76-2.58 (m, 2H), 2.17- |
| | | 2.09 (m, 3H), 2.00-1.90 (m, 4H), 1.90-1.77 (m, 1H), 1.59-1.50 (m, 6H). |
| 1.34 | 555.9 | 1H NMR (400 MHz, DMSO-d6) δ 8.98 (d, J = 5.1 Hz, 1H), 8.93-8.81 |
| | | (m, 3H), 8.69 (d, J = 15.5 Hz, 1H), 8.25 (d, J = 5.2 Hz, 1H), 8.12 (d, J = |
| | | 12.0 Hz, 1H), 6.34-6.25 (m, 1H), 2.77-2.59 (m, 2H), 2.17-2.09 (m, |
| | | 3H), 2.00-1.90 (m, 4H), 1.90-1.77 (m, 1H), 1.60-1.48 (m, 6H). |
| 1.35 | 545.1 | 1H NMR (400 MHz, DMSO-d6) δ 8.98 (d, J = 5.1 Hz, 1H), 8.87 (s, |
| | | 1H), 8.70 (d, J = 4.3 Hz, 1H), 8.25 (d, J = 5.1 Hz, 1H), 7.90-7.80 (m, |
| | | 2H), 7.57 (dd, J = 7.8, 1.8 Hz, 1H), 7.53-7.46 (m, 1H), 6.27 (d, J = 3.3 |
| | | Hz, 1H), 3.87 (s, 3H), 2.72-2.58 (m, 1H), 2.57-2.44 (m, 1H), 2.12 (s, |
| | | 3H), 1.95 (s, 3H), 1.85-1.68 (m, 2H), 1.54 (s, 3H), 1.53 (d, J = 1.6 Hz, 3H). |
| 1.36 | 523.1 | 1H NMR (400 MHz, DMSO-d6) δ 8.98 (d, J = 5.2 Hz, 1H), 8.89 (d, J = |
| | | 3.4 Hz, 1H), 8.75 (d, J = 29.5 Hz, 1H), 8.25 (d, J = 5.1 Hz, 1H), 7.53- |
| | | 7.32 (m, 5H), 6.66 (d, J = 9.9 Hz, 1H), 4.15-3.30 (m, 2H), 2.16-2.08 |
| | | (m, 3H), 1.98 (s, 3H), 1.54 (m, 6H). |
| 1.37 | 541.1 | 1H NMR (400 MHz, DMSO-d6) δ 8.97 (dd, J = 5.1, 0.8 Hz, 1H), 8.87 |
| | | (s, 1H), 8.73 (m, 1H), 8.24 (d, J = 5.2 Hz, 1H), 7.42 (dt, J = 10.0, 5.0 |
| | | Hz, 1H), 7.17 (d, J = 10.1 Hz, 1H), 6.27 (d, J = 3.9 Hz, 1H), 2.81 (ddd, |
| | | J = 14.8, 9.7, 5.5 Hz, 1H), 2.55-2.44 (m, 1H), 2. 11 (d, J = 8.9 Hz, 3H), |
| | | 1.93 (s, 3H), 1.87-1.65 (m, 2H), 1.53 (dd, J = 4.2, 1.3 Hz, 6H). |
| 1.38 | 573.0 | 1H NMR (400 MHz, DMSO-d6) δ 8.97 (d, J = 5.1 Hz, 1H), 8.90-8.82 |
| | | (m, 1H), 8.73-8.61 (m, 1H), 8.24 (d, J = 5.2 Hz, 1H), 7.76-7.61 (m, |
| | | 2H), 7.47 (t, J = 9.7 Hz, 1H), 6.29-6.22 (m, 1H), 2.72-2.52 (m, 2H), |
| | | 2.15-2.06 (m, 3H), 1.98-1.89 (m, 3H), 1.87-1.66 (m, 2H), 1.58- |
| | | 1.46 (m, 6H). |
| 1.39 | 487.1 | 1H NMR (400 MHz, DMSO-d6) δ 8.97 (d, J = 5.2 Hz, 1H), 8.86 (s, |
| | | 1H), 8.75-8.65 (m, 1H), 8.24 (d, J = 5.1 Hz, 1H), 7.40-7.18 (m, 5H), |
| | | 6.28-6.18 (m, 1H), 2.11 (s, 3H), 1.97-1.89 (m, 3H), 1.80-1.64 (m, |
| | | 2H), 1.58-1.46 (m, 6H). |
| 1.40 | 557.1 | 1H NMR (400 MHz, DMSO-d6) δ 8.97 (d, J = 5.2 Hz, 1H), 8.94-8.90 |
| | | (m, 1H), 8.90-8.83 (m, 1H), 8.71 (s, 0.5H), 8.66 (s, 0.5H), 8.24 (d, J = |
| | | 5.2 Hz, 1H), 7.97-7.89 (m, 1H), 6.35-6.29 (m, 1H), 2.97-2.80 (m, |
| | | 2H), 2.14-2.06 (m, 3H), 2.01-1.85 (m, 5H), 1.58-1.47 (m, 6H). |
| 1.41 | 557.1 | 1H NMR (400 MHz, DMSO-d6) δ 8.97 (d, J = 5.2 Hz, 1H), 8.93 (dd, J = |
| | | 5.3, 1.4 Hz, 1H), 8.87 (d, J = 2.2 Hz, 1H), 8.71 (s, 0.5H), 8.66 (s, |
| | | 0.5H), 8.24 (d, J = 5.2 Hz, 1H), 7.95-7.90 (m, 1H), 6.35-6.30 (m, |
| | | 1H), 2.97-2.80 (m, 2H), 2.13-2.08 (m, 3H), 1.99-1.87 (m, 5H), |
| | | 1.55-1.48 (m, 6H). |
| 1.42 | 488.1 | 1H NMR (400 MHz, DMSO-d6) δ 8.97 (d, J = 5.2 Hz, 1H), 8.87 (s, |
| | | 1H), 8.72-8.64 (m, 1H), 8.58-8.50 (m, 1H), 8.24 (d, J = 5.2 Hz, 1H), |
| | | 7.86-7.77 (m, 1H), 7.58-7.47 (m, 1H), 7.36-7.26 (m, 1H), 6.31- |
| | | 6.23 (m, 1H), 2.89-2.80 (m, 1H), 2.80-2.63 (m, 1H), 2.14-2.07 (m, |
| | | 3H), 1.94 (s, 3H), 1.91-1.81 (m, 1H), 1.81-1.66 (m, 1H), 1.57-1.47 |
| | | (m, 6H). |
| 1.43 | 527.1 | 1H NMR (400 MHz, DMSO-d6) δ 8.97 (d, J = 5.2 Hz, 1H), 8.90-8.80 |
| | | (m, 1H), 8.74-8.64 (m, 1H), 8.24 (d, J = 5.1 Hz, 1H), 8.15 (d, J = 2.6 |
| | | Hz, 1H), 7.93-7.52 (m, 1H), 6.55-6.46 (m, 1H), 6.28-6.19 (m, 1H), |
| | | 2.65-2.52 (m, 2H), 2.15-2.06 (m, 3H), 1.92 (s, 3H), 1.76-1.60 (m, |
| | | 2H), 1.59-1.46 (m, 6H). |
| 1.44 | 529.2 | 1H NMR (400 MHz, DMSO-d6) δ 8.97 (d, J = 5.2 Hz, 1H), 8.86 (d, J = |
| | | 1.5 Hz, 1H), 8.70 (d, J = 25.9 Hz, 1H), 8.24 (d, J = 5.1 Hz, 1H), 6.02 (d, |
| | | J = 5.3 Hz, 1H), 2.09 (d, J = 9.0 Hz, 3H), 2.20-1.95 (m, 3H), 1.95- |
| | | 1.67 (m, 4H), 1.88 (d, J = 1.9 Hz, 4H), 1.53 (d, J = 1.6 Hz, 3H), 1.52 (d, |
| | | J = 1.3 Hz, 3H), 1.48-1.01 (m, 5H). |
| 1.45 | 527.1 | 1H NMR (400 MHz, DMSO-d6) δ 8.98 (d, J = 5.2 Hz, 1H), 8.87 (s, |
| | | 1H), 8.69 (s, 1H), 8.25 (d, J = 5.2 Hz, 1H), 8.21 (s, 1H), 7.81 (s, 1H), |
| | | 7.76 (t, J = 59.4 Hz, 1H), 6.29-5.91 (m, 1H), 2.48-2.37 (m, 2H), 2.11 |
| | | (s, 3H), 1.93 (d, J = 1.0 Hz, 3H), 1.70-1.59 (m, 2H), 1.54 (s, 3H), 1.53 |
| | | (s, 3H). |
| 1.46 | 518.1 | 1H NMR (400 MHz, DMSO-d6) δ 8.98 (d, J = 5.2 Hz, 1H), 8.87 (s, |
| | | 1H), 8.70 (d, J = 7.9 Hz, 1H), 8.25 (d, J = 5.2 Hz, 1H), 7.62 (dd, J = 8.2, |
| | | 7.3 Hz, 1H), 7.09-7.00 (m, 1H), 6.65 (dd, J = 8.2, 0.7 Hz, 1H), 6.26 (s, |
| | | 1H), 3.86 (s, 3H), 2.89 (ddd, J = 8.7, 5.9, 4.1 Hz, 1H), 2.66 (ddd, J = |
| | | 8.7, 5.8, 4.2 Hz, 1H), 2.11 (s, 3H), 1.97-1.91 (m, 3H), 1.86 (ddd, J = |
| | | 8.8, 5.8, 4.2 Hz, 1H), 1.67 (ddd, J = 8.7, 5.9, 4.2 Hz, 1H), 1.54 (s, 3H), |
| | | 1.53 (s, 3H). |
| 1.47 | 530.1 | 1H NMR (400 MHz, DMSO-d6) δ 8.97 (dd, J = 5.1, 1.1 Hz, 1H), 8.86 |
| | | (s, 1H), 8.72 (m, 1H), 8.24 (dd, J = 5.1, 0.9 Hz, 1H), 7.86 (d, J = 7.0 |
| | | Hz, 1H), 7.64-7.49 (m, 1H), 7.46 (dd, J = 9.8, 8.6 Hz, 1H), 6.33- |
| | | 6.26 (m, 1H), 5.25 (d, J = 2.1 Hz, 1H), 2.70-2.54 (m, 2H), 2.11 (d, J = |
| | | 3.8 Hz, 3H), 1.94 (s, 3H), 1.93-1.84 (m, 1H), 1.83-1.67 (m, 1H), |
| | | 1.53 (dd, J = 4.6, 2.1 Hz, 6H). |
| 1.48 | 505.1 | 1H NMR (400 MHz, DMSO-d6) δ 8.97 (d, J = 5.2 Hz, 1H), 8.86 (s, |
| | | 1H), 8.69 (d, J = 2.9 Hz, 1H), 8.24 (d, J = 5.2 Hz, 1H), 7.36 (q, J = 7.4 |
| | | Hz, 1H), 7.14 (d, J = 7.8 Hz, 2H), 7.04 (t, J = 8.3 Hz, 1H), 6.24 (d, J = |
| | | 3.5 Hz, 1H), 2.54 (m, 2H), 2.11 (s, 3H), 1.93 (d, J = 1.1 Hz, 3H), 1.74 |
| | | (dt, J = 16.1, 8.0 Hz, 2H), 1.56-1.50 (m, 6H). |
| 1.49 | 505.1 | 1H NMR (400 MHz, DMSO-d6) δ 8.97 (dd, J = 5.2, 0.9 Hz, 1H), 8.86 |
| | | (s, 1H), 8.73 (m, 1H), 8.25 (dd, J = 5.2, 0.8 Hz, 1H), 7.35-7.24 (m, |
| | | 2H), 7.24-7.11 (m, 2H), 6.29 (d, J = 1.7 Hz, 1H), 2.69-2.55 (m, 2H), |
| | | 2.11 (d, J = 5.2 Hz, 3H), 1.94 (s, 3H), 1.84-1.67 (m, 2H), 1.53 (dd, J = |
| | | 4.7, 1.9 Hz, 6H). |
| 1.50 | 512.1 | 1H NMR (400 MHz, DMSO-d6) δ 8.97 (d, J = 5.2 Hz, 1H), 8.87 (s, |
| | | 1H), 8.69 (d, J = 7.7 Hz, 1H), 8.24 (d, J = 5.2 Hz, 1H), 7.78 (dt, J = 9.1, |
| | | 1.8 Hz, 1H), 7.73-7.61 (m, 2H), 7.53 (td, J = 7.8, 1.8 Hz, 1H), 6.26 (d, |
| | | J = 3.5 Hz, 1H), 2.64-2.55 (m, 2H), 2.11 (d, J = 1.9 Hz, 3H), 1.94 (t, J = |
| | | 1.1 Hz, 3H), 1.87-1.71 (m, 2H), 1.53 (dd, J = 4.7, 1.4 Hz, 6H). |
| 1.51 | 530.1 | 1H NMR (400 MHz, DMSO-d6) δ 8.98 (t, J = 4.8 Hz, 1H), 8.93-8.76 |
| | | (m, 2H), 8.68 (d, J = 14.0 Hz, 1H), 8.24 (dd, J = 5.2, 1.7 Hz, 1H), 7.69 |
| | | (dd, J = 8.8, 1.8 Hz, 2H), 7.60 (dd, J = 9.8, 7.9 Hz, 1H), 6.26 (d, J = 3.4 |
| | | Hz, 1H), 2.62 (t, J = 7.4 Hz, 2H), 2.14-1.94 (m, 6H), 1.82 (dq, J = |
| | | 22.2, 6.1 Hz, 2H), 1.59-1.48 (m, 6H). |
| 1.52 | 506.2 | 1H NMR (400 MHz, DMSO-d6) δ 8.97 (dd, J = 5.2, 0.9 Hz, 1H), 8.87 |
| | | (s, 1H), 8.74 (s, 0.5H), 8.70 (s, 0.5H), 8.56-8.54 (m, 1H), 8.43-8.38 |
| | | (m, 1H), 8.26-8.21 (m, 1H), 7.41-7.35 (m, 1H), 6.34 (s, 1H), 2.76- |
| | | 2.56 (m, 2H), 2.12-2.09 (m, 3H), 1.94 (s, 3H), 1.92-1.79 (m, 2H), |
| | | 1.55-1.47 (m, 6H). |
| 1.53 | 506.1 | 1H NMR (400 MHz, DMSO-d6) δ 8.97 (d, J = 5.2 Hz, 1H), 8.87 (d, J = |
| | | 1.0 Hz, 1H), 8.71-8.66 (m, 1H), 8.51-8.47 (m, 1H), 8.45-8.42 (m, |
| | | 1H), 8.25-8.23 (m, 1H), 7.70-7.61 (m, 1H), 6.29-6.25 (m, 1H), |
| | | 2.66-2.54 (m, 2H), 2.13-2.09 (m, 3H), 1.94 (s, 3H), 1.87-1.74 (m, |
| | | 2H), 1.58-1.46 (m, 6H). |
| 1.54 | 513.2 | 1H NMR (400 MHz, DMSO-d6) δ 9.01-8.91 (m, 1H), 8.87 (s, 1H), |
| | | 8.70 (d, J = 4.8 Hz, 1H), 8.24 (dd, J = 5.2, 1.6 Hz, 1H), 7.72-7.13 (m, |
| | | 4H), 6.60-6.45 (m, 1H), 6.16-6.01 (m, 1H), 3.59 (dd, J = 7.7, 3.3 Hz, |
| | | 2H), 3.05-2.74 (m, 4H), 2.11 (s, 3H), 1.98-1.72 (m, 3H), 1.52 (t, J = |
| | | 3.5 Hz, 6H). |
| 1.55 | 523.1 | 1H NMR (400 MHz, DMSO-d6) δ 8.98 (d, J = 5.1 Hz, 1H), 8.87 (s, |
| | | 1H), 8.69 (d, J = 7.0 Hz, 1H), 8.25 (d, J = 5.2 Hz, 1H), 7.42-7.29 (m, |
| | | 2H), 7.23-7.08 (m, 1H), 6.24 (d, J = 3.9 Hz, 1H), 2.57-2.42 (m, 2H), |
| | | 2.11 (d, J = 1.4 Hz, 3H), 1.94 (d, J = 1.2 Hz, 3H), 1.82-1.67 (m, 2H), |
| | | 1.54 (d, J = 1.0 Hz, 3H), 1.53 (d, J = 1.5 Hz, 3H). |
| 1.56 | 541.1 | 1H NMR (400 MHz, DMSO-d6) δ 8.94(d, J = 5.1 Hz, 1H), 8.87 (s, |
| | | 1H), 8.25 (d, J = 5.1 Hz, 1H), 7.45-7.38 (m, 1H), 7.18-7.13 (m, 1H), |
| | | 6.27 (d, J = 2.0 Hz, 1H), 2.65-2.41 (m, 2H), 2.13 (d, J = 2.0 Hz, 3H), |
| | | 1.94 (s, 3H), 1.89-1.64 (m, 2H), 1.54 (d, J = 1.5 Hz, 3H), 1.53 (d, J = |
| | | 2.0 Hz, 3H). |
| 1.57 | 541.1 | 1H NMR (400 MHz, DMSO-d6) δ 8.98 (dd, J = 5.1, 0.9 Hz, 1H), 8.87 |
| | | (s, 1H), 8.72 (d, J = 21.5 Hz, 1H), 8.25 (d, J = 5.1 Hz, 1H), 7.64-7.53 |
| | | (m, 1H), 7.53-7.36 (m, 1H), 6.29 (d, J = 2.0 Hz, 1H), 2.62-2.53 (m, |
| | | 1H), 2.53-2.47 (m, 1H), 2.11 (d, J = 4.2 Hz, 3H), 1.94 (s, 3H), 1.89- |
| | | 1.79 (m, 1H), 1.79-1.64 (m, 1H), 1.54 (d, J = 1.5 Hz, 3H), 1.53 (d, J = |
| | | 2.0 Hz, 3H). |
| 1.58 | 523.1 | 1H NMR (400 MHz, DMSO-d6) δ 8.98 (dd, J = 5.2, 0.8 Hz, 1H), 8.87 |
| | | (s, 1H), 8.73 (d, J = 23.0 Hz, 1H), 8.25 (d, J = 5.1 Hz, 1H), 7.39-7.30 |
| | | (m, 1H), 7.30-7.21 (m, 1H), 7.15-7.02 (m, 1H), 6.28 (d, J = 2.3 Hz, |
| | | 1H), 2.63-2.53 (m, 1H), 2.52-2.46 (m, 1H), 2.12 (d, J = 5.3 Hz, 3H), |
| | | 1.94 (s, 3H), 1.83-1.61 (m, 2H), 1.54 (d, J = 1.4 Hz, 3H), 1.53 (d, J = |
| | | 1.8 Hz, 3H). |
| 1.59 | 555.1 | 1H NMR (400 MHz, DMSO-d6) δ 8.98 (d, J = 5.1 Hz, 1H), 8.90-8.85 |
| | | (m, 1H), 8.69 (d, J = 12.6 Hz, 1H), 8.25 (d, J = 5.2 Hz, 1H), 7.76-7.42 |
| | | (m, 4H), 6.27 (d, J = 3.2 Hz, 1H), 2.76-2.55 (m, 2H), 2.12 (d, J = 1.9 |
| | | Hz, 3H), 1.95 (s, 3H), 1.91-1.69 (m, 2H), 1.54 (s, 3H), 1.53 (d, J = 1.8 |
| | | Hz, 3H). |
| 1.60 | 545.1 | 1H NMR (400 MHz, DMSO-d6) δ 8.97 (d, J = 5.2 Hz, 1H), 8.86 (s, |
| | | 1H), 8.71-8.64 (m, 1H), 8.48-8.40 (m, 1H), 8.24 (d, J = 5.2 Hz, 1H), |
| | | 8.03-7.96 (m, 1H), 6.23-6.16 (m, 1H), 2.15-2.06 (m, 3H), 1.92 (s, |
| | | 3H), 1.73-1.59 (m, 2H), 1.53 (s, 3H), 1.52 (s, 3H). |
| 1.61 | 545.1 | 1H NMR (400 MHz, DMSO-d6) δ 8.97 (d, J = 5.1 Hz, 1H), 8.86 (s, |
| | | 1H), 8.73-8.64 (m, 1H), 8.48-8.39 (m, 1H), 8.24 (d, J = 5.2 Hz, 1H), |
| | | 8.03-7.95 (m, 1H), 6.24-6.14 (m, 1H), 2.15-2.07 (m, 3H), 1.92 (s, |
| | | 3H), 1.74-1.57 (m, 2H), 1.53 (s, 3H), 1.52 (s, 3H). |
| 1.62 | 541.0 | 1H NMR (400 MHz, DMSO-d6) δ 8.97 (d, J = 5.1, 0.8 Hz, 1H), 8.87 |
| | | (d, J = 1.7 Hz, 1H), 8.73 (d, J = 29.1 Hz, 1H), 8.24 (d, J = 5.1 Hz, 1H), |
| | | 7.27-7.15 (m, 2H), 6.24 (d, J = 4.2 Hz, 1H), 2.71 (ddt, J = 10.6, 7.8, |
| | | 5.3 Hz, 1H), 2.46-2.29 (m, 1H), 2.11 (d, J = 9.7 Hz, 3H), 1.93 (s, 3H), |
| | | 1.79-1.62 (m, 2H), 1.53 (d, 6H). |
| 1.63 | 557.1 | 1H NMR (400 MHz, DMSO-d6) δ 9.12 (d, J = 1.5 Hz, 1H), 9.02-8.93 |
| | | (m, 2H), 8.87 (s, 1H), 8.69 (d, J = 11.5 Hz, 1H), 8.24 (d, J = 5.1 Hz, |
| | | 1H), 6.35 (d, J = 4.5 Hz, 1H), 3.06-2.89 (m, 2H), 2.11 (d, J = 6.2 Hz, |
| | | 3H), 2.01-1.86 (m, 5H), 1.59-1.48 (m, 6H). |
| 1.64 | 523.1 | 1H NMR (400 MHz, DMSO-d6) δ 9.03 (d, J = 5.2 Hz, 1H), 8.79 (s, |
| | | 1H), 8.05 (d, J = 5.1 Hz, 1H), 7.33 (ddd, J = 8.5, 5.4, 2.7 Hz, 2H), 7.15 |
| | | (t, J = 8.7 Hz, 2H), 6.28 (s, 1H), 2.18 (s, 3H), 2.01 (s, 3H), 2.04-1.93 |
| | | (m, 2H), 1.79-1.66 (m, 2H), 1.53 (d, J = 1.3 Hz, 6H). |
| 1.65 | 513.2 | 1H NMR (400 MHz, DMSO-d6) δ 8.97 (d, J = 5.1 Hz, 1H), 8.71 (s, |
| | | 1H), 8.24 (s, 1H), 7.98 (d, J = 5.2 Hz, 1H), 7.73 (s, 1H), 6.22 (s, 1H), |
| | | 2.64-2.53 (m, 2H), 2.15 (s, 3H), 2.01 (s, 3H), 1.74 (t, J = 7.7 Hz, 2H), |
| | | 1.59 (s, 3H), 1.58 (s, 3H). |
| 2.1 | 520.2 | 1H NMR (400 MHz, DMSO-d6) δ 8.97 (d, J = 5.2 Hz, 1H), 8.86 (s, |
| | | 1H), 8.70 (s, 1H), 8.24 (d, J = 5.2 Hz, 1H), 6.91 (dd, J = 11.4, 8.2 Hz, |
| | | 1H), 6.70-6.54 (m, 1H), 6.46-6.37 (m, 1H), 6.20 (s, 1H), 2.44-2.30 |
| | | (m, 2H), 2.10 (s, 3H), 1.92 (s, 3H), 1.71-1.45 (m, 8H). |
| 2.2 | 562.2 | 1H NMR (400 MHz, DMSO-d6) δ 9.72 (s, 1H), 8.97 (d, J = 5.1 Hz, |
| | | 1H), 8.86 (s, 1H), 8.80-8.64 (m, 1H), 8.37-8.15 (m, 1H), 7.81 (d, J = |
| | | 6.9 Hz, 1H), 7.24-7.14 (m, 1H), 7.03 (s, 1H), 6.30-6.19 (m, 1H), |
| | | 2.49-2.42 (m, 2H), 2.15-2.06 (m, 6H), 1.97-1.89 (m, 3H), 1.72- |
| | | 1.59 (m, 2H), 1.58-1.45 (m, 6H). |
| 2.3 | 588.2 | 1H NMR (400 MHz, Acetonitrile-d3) δ 8.85-8.73 (m, 1H), 8.73- |
| | | 8.63 (m, 1H), 8.58-8.08 (m, 3H, amide rotomers), 7.94 (d, J = 7.3 Hz, |
| | | 1H), 7.13-6.96 (m, 1H), 6.91-6.78 (m, 1H), 5.96-5.82 (m, 1H), |
| | | 2.48-2.37 (m, 4H), 2.37-2.25 (m, 1H), 2.14-2.00 (m, 3H), 1.78- |
| | | 1.60 (m, 2H), 1.57-1.40 (m, 7H), 0.91-0.67 (m, 4H). |
| 3.1 | 534.2 | 1H NMR (400 MHz, Chloroform-d) δ 8.86 (s, 1H), 7.98 (s, 1H), 7.58 |
| | | (s, 1H), 7.46 (ddd, J = 7.8, 6.0, 1.9 Hz, 1H), 7.37 (t, J = 7.7 Hz, 1H), |
| | | 7.23-7.16 (m, 2H), 7.02 (td, J = 9.6, 9.1, 2.6 Hz, 2H), 5.81 (d, J = 1.0 |
| | | Hz, 1H), 3.16 (s, 3H), 3.01-2.93 (m, 3H), 2.61 (ddd, J = 8.7, 5.8, 4.6 |
| | | Hz, 1H), 2.37 (dt, J = 9.1, 5.7 Hz, 1H), 2.23 (s, 3H), 1.98 (s, 3H), 1.60 |
| | | (dt, J = 8.8, 5.9 Hz, 1H), 1.52 (dt, J = 9.0, 5.7 Hz, 1H). |
| 3.2 | 566.1 | 1H NMR (400 MHz, DMSO-d6) δ 8.84 (s, 1H), 8.08-7.94 (m, 1H), |
| | | 7.93-7.73 (m, 1H), 7.55-7.34 (m, 4H), 7.34-7.20 (m, 1H), 6.46 (s, |
| | | 1H), 3.01 (s, 3H), 2.88 (s, 3H), 2.44-2.26 (m, 1H), 2.06 (d, J = 6.2 Hz, |
| | | 3H), 1.96 (s, 3H), 1.75-1.59 (m, 1H), 1.59-1.47 (m, 1H), 1.22 (d, J = |
| | | 7.8 Hz, 3H). |
| 3.3 | 551.2 | 1H NMR (400 MHz, DMSO-d6) δ 8.83 (s, 1H), 8.56 (d, J = 1.9 Hz, |
| | | 1H), 8.48 (d, J = 2.3 Hz, 1H), 8.01 (td, J = 7.6, 2.1 Hz, 1H), 7.86 (t, J = |
| | | 2.2 Hz, 1H), 7.79 (d, J = 1.9 Hz, 1H), 7.50-7.37 (m, 2H), 6.24 (s, 1H), |
| | | 3.02 (s, 3H), 2.88 (s, 3H), 2.63-2.53 (m, 2H), 2.07 (s, 3H), 1.94 (s, |
| | | 3H), 1.83 (dt, J = 8.7, 5.7 Hz, 1H), 1.75 (dt, J = 9.0, 5.7 Hz, 1H). |
| 3.4 | 530.2 | 1H NMR (400 MHz, DMSO-d6) δ 8.83 (s, 1H), 8.01 (td, J = 7.6, 2.2 |
| | | Hz, 1H), 7.79 (d, J = 1.9 Hz, 1H), 7.50-7.38 (m, 2H), 7.24-7.13 (m, |
| | | 4H), 6.24 (s, 1H), 3.02 (s, 3H), 2.88 (s, 3H), 2.40-2.29 (m, 4H), 2.08 |
| | | (s, 3H), 1.95 (s, 3H), 1.73-1.60 (m, 2H). |
| 3.5 | 552.1 | 1H NMR (400 MHz, DMSO-d6) δ 8.83 (s, 1H), 8.01 (td, J = 7.5, 2.2 |
| | | Hz, 1H), 7.78 (d, J = 1.9 Hz, 1H), 7.51-7.30 (m, 3H), 7.10 (t, J = 8.2 |
| | | Hz, 2H), 6.22 (s, 1H), 3.02 (s, 3H), 2.88 (s, 3H), 2.78 (dt, J = 8.7, 5.5 |
| | | Hz, 1H), 2.48-2.41 (m, 1H), 2.08 (s, 3H), 1.94 (s, 3H), 1.77 (dt, J = |
| | | 8.8, 5.7 Hz, 1H), 1.67 (dt, J = 10.3, 5.4 Hz, 1H). |
| 3.6 | 550.1 | 1H NMR (400 MHz, DMSO-d6) δ 8.83 (s, 1H), 8.05-7.94 (m, 1H), |
| | | 7.78 (d, J = 2.1 Hz, 1H), 7.51-7.21 (m, 6H), 6.20 (s, 1H), 3.02 (s, 3H), |
| | | 2.88 (s, 3H), 2.07 (s, 3H), 1.93 (s, 3H), 1.78-1.62 (m, 2H). |
| 3.7 | 550.1 | 1H NMR (400 MHz, DMSO-d6) δ 8.83 (s, 1H), 8.06-7.92 (m, 1H), |
| | | 7.78 (s, 1H), 7.52-7.17 (m, 6H), 6.20 (s, 1H), 3.02 (s, 3H), 2.88 (s, |
| | | 3H), 2.07 (s, 3H), 1.94 (s, 3H), 1.79-1.62 (m, 2H). |
| 3.8 | 552.1 | 1H NMR (400 MHz, DMSO-d6) δ 8.83 (s, 1H), 8.01 (td, J = 7.6, 2.2 |
| | | Hz, 1H), 7.78 (d, J = 1.9 Hz, 1H), 7.50-7.37 (m, 2H), 7.25 (td, J = 9.3, |
| | | 4.7 Hz, 1H), 7.21-7.06 (m, 2H), 6.25 (s, 1H), 3.02 (s, 3H), 2.88 (s, |
| | | 3H), 2.57 (td, J = 6.7, 3.4 Hz, 2H), 2.07 (s, 3H), 1.94 (s, 3H), 1.85- |
| | | 1.66 (m, 2H). |
| 3.9 | 520.1 | 1H NMR (400 MHz, DMSO-d6) δ 8.82 (s, 1H), 8.01 (td, J = 7.6, 2.2 |
| | | Hz, 1H), 7.77 (d, J = 1.9 Hz, 1H), 7.57 (d, J = 2.2 Hz, 1H), 7.51-7.37 |
| | | (m, 2H), 6.20-6.09 (m, 2H), 3.77 (s, 3H), 3.01 (s, 3H), 2.88 (s, 3H), |
| | | 2.06 (s, 3H), 1.92 (s, 3H), 1.67-1.50 (m, 2H). |
| 3.10 | 562.2 | 1H NMR (400 MHz, DMSO-d6) δ 8.83 (d, J = 0.7 Hz, 1H), 8.01 (td, J = |
| | | 7.6, 2.1 Hz, 1H), 7.77 (d, J = 2.0 Hz, 1H), 7.76 (d, J = 0.8 Hz, 1H), |
| | | 7.47 (ddd, J = 7.9, 5.8, 2.1 Hz, 1H), 7.43 (d, 1H), 7.41-7.37 (m, 1H), |
| | | 6.21-5.97 (m, 1H), 3.02 (s, 3H), 2.89 (d, J = 1.0 Hz, 3H), 2.39-2.25 |
| | | (m, 2H), 2.07 (s, 3H), 1.92 (d, J = 0.9 Hz, 3H), 1.61-1.52 (m, 2H), |
| | | 1.50 (s, 9H). |
| 3.11 | 569.2 | 1H NMR (400 MHz, DMSO-d6) δ 8.83 (s, 1H), 8.49 (d, J = 1.9 Hz, |
| | | 1H), 8.08 (dd, J = 9.7, 2.0 Hz, 1H), 8.01 (td, J = 7.6, 2.1 Hz, 1H), 7.79 |
| | | (d, J = 1.9 Hz, 1H), 7.49-7.40 (m, 2H), 6.32 (s, 1H), 3.02 (s, 3H), 2.95- |
| | | 2.82 (m, 5H), 2.07 (s, 3H), 1.95 (s, 3H), 1.87-1.80 (m, 1H), 1.79- |
| | | 1.71 (m, 1H). (Expect 24, observe 24) |
| 3.12 | 567.3 | 1H NMR (400 MHz, DMSO-d6) δ 8.84 (s, 1H), 8.76 (d, J = 2.0 Hz, |
| | | 1H), 8.66 (t, J = 1.6 Hz, 1H), 8.02 (td, J = 7.6, 2.1 Hz, 1H), 7.91 (s, |
| | | 1H), 7.79 (d, J = 1.9 Hz, 1H), 7.50-7.45 (m, 1H), 7.43 (t, J = 7.5 Hz, |
| | | 1H), 7.14 (t, J = 55.3 Hz, 1H), 6.26 (s, 1H), 3.02 (s, 3H), 2.89 (s, 3H), |
| | | 2.70-2.64 (m, 1H), 2.64-2.57 (m, 1H), 2.08 (s, 3H), 1.95 (s, 3H), |
| | | 1.91-1.82 (m, 1H), 1.82-1.72 (m, 1H). |
| 3.13 | 554.1 | 1H NMR (400 MHz, DMSO-d6) δ 9.11-9.05 (m, 1H), 8.13-8.09 (m, |
| | | 1H), 8.06-7.98 (m, 1H), 7.55-7.49 (m, 1H), 7.46 (t, J = 7.6 Hz, 1H), |
| | | 7.37-7.27 (m, 2H), 7.20-7.11 (m, 2H), 6.25-6.19 (m, 1H), 3.05- |
| | | 3.00 (m, 3H), 2.89 (s, 3H), 2.58-2.43 (m, 2H), 1.98 (s, 3H), 1.76- |
| | | 1.64 (m, 2H). |
| 3.14 | 555.1 | 1H NMR (400 MHz, DMSO-d6) δ 9.09 (d, J = 1.3 Hz, 1H), 8.52-8.45 |
| | | (m, 1H), 8.44 (d, J = 2.7 Hz, 1H), 8.14-8.09 (m, 1H), 8.06-7.99 (m, |
| | | 1H), 7.71-7.63 (m, 1H), 7.55-7.49 (m, 1H), 7.46 (t, J = 7.6 Hz, 1H), |
| | | 6.26 (d, J = 2.9 Hz, 1H), 3.03 (d, J = 1.3 Hz, 3H), 2.89 (s, 3H), 2.71- |
| | | 2.53 (m, 2H), 1.98 (s, 3H), 1.90-1.73 (m, 2H). |
| 3.15 | 554.1 | 1H NMR (400 MHz, DMSO-d6) δ 8.89 (d, J = 5.1 Hz, 1H), 7.85-7.77 |
| | | (m, 1H), 7.72-7.63 (m, 1H), 7.56 (td, J = 7.0, 6.3, 1.8 Hz, 1H), 7.46 (t, |
| | | J = 7.6 Hz, 1H), 7.38-7.26 (m, 2H), 7.19-7.08 (m, 2H), 6.23 (d, J = |
| | | 2.1 Hz, 1H), 3.02 (s, 3H), 2.88 (s, 3H), 2.61-2.37 (m, 2H), 1.97 (s, |
| | | 3H), 1.76-1.59 (m, 2H). |
| 3.16 | 571.2 | 1H NMR (400 MHz, DMSO-d6) δ 9.11-9.06 (m, 1H), 8.59-8.52 (m, |
| | | 1H), 8.51-8.47 (m, 1H), 8.15-8.09 (m, 1H), 8.06-7.99 (m, 1H), |
| | | 7.90-7.83 (m, 1H), 7.56-7.49 (m, 1H), 7.46 (t, J = 7.6 Hz, 1H), 6.28- |
| | | 6.22 (m, 1H), 3.05-2.99 (m, 3H), 2.89 (s, 3H), 2.68-2.52 (m, 2H), |
| | | 1.98 (s, 3H), 1.91-1.71 (m, 2H). |
| 3.17 | 584.9 | 1H NMR (400 MHz, Methanol-d4) δ 8.91-8.71 (m, 3H), 8.17-8.00 |
| | | (m, 2H), 7.83-7.74 (m, 1H), 7.56-7.41 (m, 2H), 6.31 (s, 1H), 3.15 (s, |
| | | 3H), 3.01 (s, 3H), 2.85-2.74 (m, 1H), 2.74-2.61 (m, 1H), 2.19 (s, |
| | | 3H), 2.07 (s, 3H), 1.95-1.81 (m, 2H). |
| 3.18 | 541.9 | 1H NMR (400 MHz, Methanol-d4) δ 8.88-8.76 (m, 3H), 8.16-8.09 |
| | | (m, 1H), 8.09-8.01 (m, 1H), 7.81-7.73 (m, 1H), 7.55-7.42 (m, 2H), |
| | | 6.30 (s, 1H), 3.16 (s, 3H), 3.01 (s, 3H), 2.83-2.73 (m, 1H), 2.69-2.54 |
| | | (m, 1H), 2.19 (d, J = 2.4 Hz, 3H), 2.07 (s, 3H), 1.91-1.78 (m, 2H). |
| 3.19 | 591.9 | 1H NMR (400 MHz, Methanol-d4) δ 8.85 (s, 1H), 8.04 (td, J = 7.6, 2.0 |
| | | Hz, 1H), 7.82 (s, 1H), 7.57-7.38 (m, 3H), 7.16 (m, 1H), 7.01 (m, 1H), |
| | | 6.23 (s, 1H), 3.15 (s, 3H), 3.01 (s, 3H), 2.63 (m, 1H), 2.54 (m, 1H), |
| | | 2.20 (s, 3H), 2.06 (s, 3H), 1.70 (m 2H), 1.60 (s, 6H) |
| 3.20 | 551.8 | 1H NMR (400 MHz, Methanol-d4) δ 8.84 (s, 1H), 8.04 (td, J = 7.5, 2.0 |
| | | Hz, 1H), 7.80 (d, J = 1.9 Hz, 1H), 7.71 (t, J = 7.8 Hz, 1H), 7.54-7.41 |
| | | (m, 2H), 7.38 (dd, J = 7.6, 0.8 Hz, 1H), 7.26 (dd, J = 7.9, 0.8 Hz, 1H), |
| | | 6.26 (d, J = 1.0 Hz, 1H), 3.16 (s, 3H), 3.01 (d, J = 1.1 Hz, 3H), 2.94 |
| | | (ddd, J = 8.8, 6.1, 4.3 Hz, 1H), 2.63 (ddd, J = 8.7, 5.9, 4.3 Hz, 1H), 2.18 |
| | | (s, 3H), 2.06 (s, 3H), 1.92 (ddd, J = 8.9, 5.9, 4.8 Hz, 1H), 1.72 (ddd, J = |
| | | 8.8, 6.1, 4.7 Hz, 1H). |
| 3.21 | 582.2 | 1H NMR (400 MHz, DMSO-d6) δ 9.13 (s, 1H), 8.82 (s, 1H), 8.20 (s, |
| | | 1H), 7.80 (s, 1H), 7.77 (dd, J = 7.7, 1.9 Hz, 1H), 7.75 (t, J = 59.3 Hz, |
| | | 1H), 7.69 (s, 1H), 7.59-7.50 (m, 1H), 7.30 (t, J = 7.9 Hz, 1H), 6.15 (s, |
| | | 1H), 2.48-2.33 (m, 2H), 2.07 (s, 3H), 1.93 (s, 3H), 1.71-1.54 (m, |
| | | 2H), 1.41 (s, 3H), 1.10 (q, J = 3.6 Hz, 2H), 0.67 (q, J = 3.7 Hz, 2H). |
| 3.22 | 610.9 | 1H NMR (400 MHz, DMSO-d6) δ 9.13 (s, 1H), 8.89 (d, J = 2.1 Hz, |
| | | 1H), 8.83 (s, 2H), 8.16-8.11 (m, 1H), 7.79 (td, J = 7.5, 1.8 Hz, 1H), |
| | | 7.71 (s, 1H), 7.56 (td, J = 7.5, 1.8 Hz, 1H), 7.31 (t, J = 7.9 Hz, 1H), 6.26 |
| | | (s, 1H), 2.73-2.60 (m, 2H), 2.07 (s, 3H), 1.95 (s, 3H), 1.91 (dd, J = |
| | | 8.8, 5.8 Hz, 1H), 1.79 (dt, J = 8.8, 5.7 Hz, 1H), 1.41 (s, 3H), 1.10 (q, J = |
| | | 3.6 Hz, 2H), 0.67 (q, J = 3.7 Hz, 2H). |
| 3.23 | 559.9 | 1H NMR (400 MHz, DMSO-d6) δ 9.13 (s, 1H), 8.82 (s, 1H), 7.78 (td, J = |
| | | 7.5, 1.8 Hz, 1H), 7.70 (s, 1H), 7.56 (td, J = 7.6, 1.8 Hz, 2H), 7.38- |
| | | 7.25 (m, 3H), 7.22-7.08 (m, 2H), 6.20 (s, 1H), 2.49-2.43 (m, 2H), |
| | | 2.08 (s, 3H), 1.94 (s, 3H), 1.72-1.63 (m, 2H), 1.41 (s, 3H), 1.10 (q, J = |
| | | 3.6 Hz, 2H), 0.67 (q, J = 3.7 Hz, 2H). |
| 3.24 | 582.1 | 1H NMR (400 MHz, DMSO-d6) δ 9.13 (s, 1H), 8.84 (s, 1H), 8.14- |
| | | 8.08 (m, 1H), 7.86-7.75 (m, 2H), 7.75-7.63 (m, 2H), 7.62-7.53 (m, |
| | | 1H), 7.49-7.42 (m, 1H), 7.30 (t, J = 7.9 Hz, 1H), 7.20 (t, J = 7.5 Hz, |
| | | 1H), 6.32 (d, J = 3.2 Hz, 1H), 4.57-4.17 (m, 1H), 3.00-2.79 (m, 1H), |
| | | 2.22-2.05 (m, 4H), 2.03-1.80 (m, 4H), 1.43-1.37 (m, 3H), 1.13- |
| | | 1.00 (m, 2H), 0.76-0.60 (m, 2H). |
| 3.25 | 541.2 | 1H NMR (400 MHz, DMSO-d6) δ 9.13-9.06 (m, 1H), 8.52-8.46 (m, |
| | | 1H), 8.46-8.42 (m, 1H), 8.41-8.33 (m, 1H), 8.10-8.01 (m, 2H), |
| | | 7.75-7.69 (m, 1H), 7.68-7.63 (m, 1H), 7.44 (t, J = 7.7 Hz, 1H), 6.27 |
| | | (s, 1H), 2.81-2.76 (m, 3H), 2.69-2.54 (m, 2H), 1.99 (s, 3H), 1.88- |
| | | 1.75 (m, 2H). |
| 3.26 | 557.1 | 1H NMR (400 MHz, DMSO-d6) δ 9.11-9.07 (m, 1H), 8.60-8.52 (m, |
| | | 1H), 8.51-8.47 (m, 1H), 8.41-8.31 (m, 1H), 8.10-8.05 (m, 1H), |
| | | 8.05-8.01 (m, 1H), 7.90-7.83 (m, 1H), 7.75-7.68 (m, 1H), 7.44 (t, J = |
| | | 7.7 Hz, 1H), 6.27 (s, 1H), 2.82-2.76 (m, 3H), 2.65-2.57 (m, 2H), |
| | | 1.99 (s, 3H), 1.91-1.74 (m, 2H). |
| 3.27 | 564.3 | 1H NMR (400 MHz, DMSO-d6) δ 9.17 (s, 1H), 8.73 (s, 1H), 7.62 (td, J = |
| | | 7.6, 1.8 Hz, 1H), 7.58 (d, J = 2.2 Hz, 1H), 7.48 (ddd, J = 8.1, 6.4, 1.8 |
| | | Hz, 1H), 7.34 (t, J = 7.8 Hz, 1H), 6.25-6.18 (m, 1H), 6.15 (d, J = 2.2 |
| | | Hz, 1H), 3.78 (s, 3H), 2.57-2.42 (m, 1H), 2.15 (s, 3H), 1.98 (s, 3H), |
| | | 1.73-1.53 (m, 2H), 1.41 (s, 3H), 1.09 (q, J = 3.6 Hz, 2H), 0.66 (q, J = |
| | | 3.7 Hz, 2H). |
| 3.28 | 578.2 | 1H NMR (400 MHz, DMSO-d6) δ 9.18 (s, 1H), 8.73 (d, J = 0.8 Hz, |
| | | 1H), 7.62 (td, J = 7.6, 1.8 Hz, 1H), 7.55-7.41 (m, 1H), 7.38-7.27 (m, |
| | | 3H), 7.22-7.07 (m, 2H), 6.29-6.12 (m, 1H), 2.59-2.53 (m, 1H), |
| | | 2.54-2.42 (m, 1H), 2.15 (s, 3H), 2.00 (s, 3H), 1.83-1.71 (m, 2H), |
| | | 1.41 (s, 3H), 1.14-1.04 (m, 2H), 0.71-0.62 (m, 2H). |
| 3.29 | 553.2 | 1H NMR (400 MHz, DMSO-d6) δ 8.76 (d, J = 0.8 Hz, 1H), 8.49 (d, J = |
| | | 1.8 Hz, 1H), 8.44 (d, J = 2.7 Hz, 1H), 7.77 (td, J = 7.3, 1.9 Hz, 1H), |
| | | 7.67 (dt, J = 10.4, 2.4 Hz, 1H), 7.59-7.52 (m, 1H), 7.48 (t, J = 7.6 Hz, |
| | | 1H), 6.32 (s, 1H), 3.02 (s, 3H), 2.87 (d, J = 1.0 Hz, 3H), 2.66-2.58 (m, |
| | | 2H), 2.16 (s, 3H), 2.01 (s, 3H), 1.88-1.76 (m, 2H). |
| 3.30 | 545.2 | 1H NMR (400 MHz, DMSO-d6) δ 8.42 (d, J = 4.4 Hz, 1H), 7.74-7.59 |
| | | (m, 2H), 7.59-7.48 (m, 2H), 7.43 (d, J = 1.5 Hz, 1H), 7.38-7.23 (m, |
| | | 3H), 7.23-7.07 (m, 2H), 6.16 (s, 1H), 2.91-2.80 (m, 1H), 2.58-2.51 |
| | | (m, 1H), 2.48-2.43 (m, 1H), 2.03 (s, 3H), 1.92 (s, 3H), 1.66 (t, J = 7.4 |
| | | Hz, 2H), 0.77-0.66 (m, 2H), 0.58-0.46 (m, 2H). |
| 3.31 | 578.1 | 1H NMR (400 MHz, Methanol-d4) δ 8.77-8.60 (m, 1H), 8.07 (s, 1H), |
| | | 8.02-7.89 (m, 1H), 7.87-7.77 (m, 1H), 7.64-7.54 (m, 2H), 6.24 (s, |
| | | 1H), 3.79-3.59 (m, 1H), 2.82-2.70 (m, 1H), 2.65-2.48 (m, 1H), |
| | | 2.26-2.20 (m, 3H), 2.09 (s, 3H), 1.81-1.62 (m, 2H), 1.12-0.96 (m, 4H). |
| 3.32 | 570.2 | 1H NMR (400 MHz, DMSO-d6) δ 8.71 (s, 1H), 8.18 (s, 1H), 7.70 (s, |
| | | 1H), 7.52-7.41 (m, 2H), 7.28 (t, J = 7.7 Hz, 1H), 6.25 (s, 1H), 2.62- |
| | | 2.54 (m, 2H), 2.13 (s, 3H), 1.98 (s, 3H), 1.80 (s, 3H), 1.74-1.65 (m, |
| | | 2H), 1.62 (s, 6H). |
| 3.33 | 611.0 | 1H NMR (400 MHz, DMSO-d6) δ 8.76 (s, 1H), 8.19 (s, 1H), 7.91- |
| | | 7.47 (m, 5H), 6.22 (s, 1H), 2.47-2.35 (m, 2H), 2.15 (s, 3H), 2.13 (s, |
| | | 3H), 2.05 (d, J = 1.4 Hz, 3H), 1.97 (s, 3H), 1.93 (s, 3H), 1.64 (dd, J = |
| | | 8.5, 6.3 Hz, 2H). |
| 3.34 | 597.0 | 1H NMR (400 MHz, DMSO-d6) δ 8.76 (s, 1H), 8.19 (s, 1H), 7.93- |
| | | 7.50 (m, 5H), 6.22 (s, 1H), 6.10 (s, 1H), 2.46-2.36 (m, 2H), 2.18 (s, |
| | | 3H), 2.15 (s, 3H), 2.13 (s, 3H), 1.97 (s, 3H), 1.64 (dd, J = 8.5, 6.2 Hz, 2H). |
| 3.35 | 576.8 | 1H NMR (400 MHz, Methanol-d4) δ 8.73 (d, J = 0.8 Hz, 1H), 8.48 (s, |
| | | 1H), 8.42 (d, J = 2.7 Hz, 1H), 7.95 (ddd, J = 8.1, 6.5, 1.8 Hz, 1H), 7.77 |
| | | (ddd, J = 8.7, 7.0, 1.8 Hz, 1H), 7.66-7.57 (m, 1H), 7.46-7.37 (m, |
| | | 1H), 6.32 (s, 1H), 2.77 (ddd, J = 8.5, 6.4, 4.6 Hz, 1H), 2.65 (q, J = 6.5, |
| | | 5.8 Hz, 1H), 2.44 (d, J = 1.3 Hz, 3H), 2.43 (s, 3H), 2.25 (s, 3H), 2.10 (s, |
| | | 3H), 1.89-1.77 (m, 2H). |
| 3.36 | 609.1 | 1H NMR (400 MHz, Chloroform-d) δ 8.63 (d, J = 0.8 Hz, 1H), 8.19 (s, |
| | (M + Na)+ | 1H), 7.92 (ddd, J = 8.2, 6.9, 1.8 Hz, 1H), 7.74 (ddd, J = 8.1, 6.9, 1.8 Hz, |
| | | 1H), 7.52 (t, J = 7.8 Hz, 1H), 7.48 (s, 1H), 7.29 (s, 1H), 7.18 (t, J = 57.8 |
| | | Hz, 1H), 5.75 (s, 1H), 2.49 (ddd, J = 8.5, 5.7, 4.8 Hz, 1H), 2.25 (s, 3H), |
| | | 2.18 (ddd, J = 8.9, 6.4, 5.0 Hz, 1H), 2.00 (s, 3H), 1.52-1.41 (m, 2H). |
| 3.37 | 605.2 | 1H NMR (400 MHz, Chloroform-d) δ 8.64 (s, 1H), 8.19 (s, 1H), 7.96- |
| | | 7.89 (m, 1H), 7.78-7.70 (m, 1H), 7.56-7.48 (m, 1H), 7.30 (d, J = 3.3 |
| | | Hz, 1H), 7.18 (t, J = 57.8 Hz, 1H), 5.74 (s, 1H), 2.55 (ddd, J = 8.7, 5.7, |
| | | 4.9 Hz, 1H), 2.26 (s, 3H), 2.09-2.03 (m, 1H), 2.00 (s, 3H), 1.52 (dt, J = |
| | | 8.7, 5.9 Hz, 1H), 1.44 (dt, J = 9.0, 5.6 Hz, 1H). |
| 3.38 | 569.3 | 1H NMR (400 MHz, DMSO-d6) δ 9.09 (s, 1H), 8.14 (ddd, J = 8.2, 6.7, |
| | | 1.8 Hz, 1H), 8.09 (ddd, J = 8.3, 6.7, 1.8 Hz, 1H), 7.72 (s, 1H), 7.69 (t, J = |
| | | 7.8 Hz, 1H), 6.31 (d, J = 1.0 Hz, 1H), 3.40 (s, 3H), 2.67-2.55 (m, |
| | | 2H), 2.07 (s, 3H), 1.71 (dd, J = 8.2, 6.9 Hz, 2H). |
| 3.39 | 565.2 | 1H NMR (400 MHz, DMSO-d6) δ 8.85 (d, J = 0.8 Hz, 1H), 8.07-7.98 |
| | | (m, 2H), 7.71 (s, 1H), 7.68-7.61 (m, 1H), 6.28 (d, J = 1.0 Hz, 1H), |
| | | 3.39 (s, 3H), 2.63-2.56 (m, 2H), 2.18-2.15 (m, 3H), 1.97 (d, J = 0.9 |
| | | Hz, 3H), 1.76-1.58 (m, 2H). |
| 4.1 | 572.2 | 1H NMR (400 MHz, DMSO-d6) δ 9.12 (s, 1H), 8.81 (s, 1H), 7.83- |
| | | 7.73 (m, 1H), 7.73-7.64 (m, 1H), 7.60-7.51 (m, 1H), 7.42-7.33 (m, |
| | | 1H), 7.29 (t, J = 7.9 Hz, 1H), 6.10 (s, 1H), 4.01 (t, J = 7.2 Hz, 2H), 2.93- |
| | | 2.77 (m, 2H), 2.58-2.53 (m, 2H), 2.42-2.30 (m, 1H), 2.30-2.18 |
| | | (m, 1H), 2.10-2.01 (m, 3H), 1.99-1.85 (m, 3H), 1.59-1.45 (m, 2H), |
| | | 1.40 (s, 3H), 1.09 (q, J = 3.6 Hz, 2H), 0.66 (q, J = 3.7 Hz, 2H). |
| 4.2 | 517.2 | 1H NMR (400 MHz, DMSO-d6) δ 8.86-8.74 (m, 2H), 8.69-8.62 (m, |
| | | 1H), 8.22-8.10 (m, 1H), 8.06-7.99 (m, 1H), 7.81-7.70 (m, 2H), |
| | | 7.49-7.39 (m, 2H), 6.26 (s, 1H), 3.02 (s, 3H), 2.88 (s, 3H), 2.72-2.59 |
| | | (m, 2H), 2.08-2.04 (m, 3H), 1.95 (s, 3H), 1.90-1.78 (m, 2H). |
| 4.3 | 517.2 | 1H NMR (400 MHz, DMSO-d6) δ 8.84 (d, J = 2.2 Hz, 1H), 8.78-8.71 |
| | | (m, 2H), 8.07-7.97 (m, 1H), 7.90-7.81 (m, 2H), 7.81-7.72 (m, 1H), |
| | | 7.50-7.39 (m, 2H), 6.30 (s, 1H), 3.02 (s, 3H), 2.88 (t, J = 1.2 Hz, 3H), |
| | | 2.84-2.65 (m, 2H), 2.09-1.92 (m, 8H). |
| 4.4 | 535.1 | 1H NMR (400 MHz, DMSO-d6) δ 8.83 (s, 1H), 8.50 (d, J = 2.9 Hz, |
| | | 1H), 8.05-7.96 (m, 1H), 7.77 (d, J = 8.9 Hz, 1H), 7.67 (td, J = 8.7, 2.8 |
| | | Hz, 1H), 7.54 (ddd, J = 12.8, 8.7, 4.4 Hz, 1H), 7.50-7.37 (m, 2H), |
| | | 6.22 (s, 1H), 3.04-2.98 (m, 3H), 2.92-2.84 (m, 3H), 2.81-2.68 (m, |
| | | 2H), 2.10-2.02 (m, 3H), 1.94 (s, 3H), 1.83-1.62 (m, 2H). |
| 4.5 | 546.2 | 1H NMR (400 MHz, DMSO-d6) δ 8.82 (s, 1H), 8.01 (td, J = 7.6, 2.1 |
| | | Hz, 1H), 7.83-7.73 (m, 1H), 7.52-7.31 (m, 3H), 6.11 (s, 1H), 4.00 (t, |
| | | J = 7.2 Hz, 2H), 3.01 (s, 3H), 2.92-2.77 (m, 5H), 2.58-2.52 (m, 2H), |
| | | 2.42-2.28 (m, 1H), 2.27-2.13 (m, 1H), 2.09-2.03 (m, 3H), 1.96- |
| | | 1.87 (m, 3H), 1.64-1.42 (m, 2H). |
| 4.6 | 546.2 | 1H NMR (400 MHz, DMSO-d6) δ 8.84 (s, 1H), 8.06-7.96 (m, 1H), |
| | | 7.82-7.70 (m, 1H), 7.55-7.34 (m, 3H), 6.23 (s, 1H), 4.34-4.05 (m, |
| | | 2H), 3.13 (t, J = 7.7 Hz, 2H), 3.01 (s, 3H), 2.88 (s, 3H), 2.76-2.63 (m, |
| | | 2H), 2.56-2.51 (m, 2H), 2.10-2.01 (m, 3H), 1.94 (s, 3H), 1.72-1.56 |
| | | (m, 2H). |
| 4.7 | 548.2 | 1H NMR (400 MHz, DMSO-d6) δ 8.82 (s, 1H), 8.13-7.93 (m, 1H), |
| | | 7.86-7.72 (m, 1H), 7.55-7.36 (m, 2H), 7.27 (d, J = 8.4 Hz, 1H), 6.08 |
| | | (s, 1H), 5.07 (t, J = 7.8 Hz, 2H), 4.22 (t, J = 7.9 Hz, 2H), 3.01 (s, 3H), |
| | | 2.88 (s, 3H), 2.37-2.28 (m, 1H), 2.20-2.00 (m, 4H), 1.93-1.84 (m, |
| | | 3H), 1.59-1.39 (m, 2H). |
| 4.8 | 561.6 | 1H NMR (400 MHz, DMSO-d6) δ 8.82 (s, 1H), 8.00 (t, J = 7.6 Hz, |
| | | 1H), 7.81-7.70 (m, 1H), 7.53-7.36 (m, 2H), 7.18 (d, J = 6.5 Hz, 1H), |
| | | 6.07 (d, J = 3.0 Hz, 1H), 4.38-4.19 (m, 2H), 4.05 (t, J = 6.0 Hz, 2H), |
| | | 3.01 (s, 3H), 2.88 (s, 3H), 2.38-2.25 (m, 2H), 2.20-2.11 (m, 2H), |
| | | 2.10-2.02 (m, 3H), 1.90 (s, 3H), 1.60-1.35 (m, 2H). |
| 4.9 | 577.1 | 1H NMR (400 MHz, DMSO-d6) δ 9.12 (s, 1H), 8.82 (s, 1H), 8.60- |
| | | 8.53 (m, 1H), 8.48 (d, J = 2.3 Hz, 1H), 7.89-7.82 (m, 1H), 7.82-7.74 |
| | | (m, 1H), 7.72-7.63 (m, 1H), 7.58-7.48 (m, 1H), 7.30 (t, J = 7.9 Hz, |
| | | 1H), 6.23 (s, 1H), 2.69-2.53 (m, 2H), 2.07 (s, 3H), 1.94 (s, 3H), 1.87- |
| | | 1.65 (m, 2H), 1.41 (s, 3H), 1.09 (q, J = 3.6 Hz, 2H), 0.66 (q, J = 3.7 Hz, 2H). |
| 4.10 | 571.2 | 1H NMR (400 MHz, DMSO-d6) δ 8.83 (s, 1H), 8.54 (s, 1H), 8.05- |
| | | 7.97 (m, 1H), 7.78 (s, 1H), 7.53 (t, J = 7.6 Hz, 1H), 7.48-7.34 (m, |
| | | 3H), 7.16 (d, J = 7.8 Hz, 1H), 6.24 (s, 1H), 4.33 (s, 2H), 3.93 (dd, J = |
| | | 13.3, 6.8 Hz, 1H), 3.02 (s, 3H), 2.88 (d, J = 0.9 Hz, 3H), 2.63-2.56 (m, |
| | | 1H), 2.07 (s, 3H), 1.96 (s, 3H), 1.77 (dt, J = 13.5, 7.0 Hz, 2H). |
| 4.11 | 571.3 | 1H NMR (400 MHz, DMSO-d6) δ 8.83 (s, 1H), 8.59 (s, 1H), 8.01 (td, J = |
| | | 7.6, 2.2 Hz, 1H), 7.79 (d, J = 1.9 Hz, 1H), 7.58-7.54 (m, 1H), 7.51- |
| | | 7.43 (m, 3H), 7.41-7.37 (m, 1H), 6.26 (s, 1H), 4.49 (d, J = 17.6 Hz, |
| | | 1H), 4.42 (d, J = 17.9 Hz, 1H), 3.02 (s, 3H), 2.88 (d, J = 1.0 Hz, 3H), |
| | | 2.57-2.45(m, 2H), 2.08 (s, 3H), 1.99-1.90 (m, 3H), 1.82-1.63 (m, 2H). |
| 4.12 | 578.2 | 1H NMR (400 MHz, DMSO-d6) δ 9.33-9.23 (m, 1H), 9.12 (s, 1H), |
| | | 8.82 (s, 1H), 7.92-7.82 (m, 1H), 7.78 (t, J = 7.6 Hz, 1H), 7.72-7.62 |
| | | (m, 1H), 7.55 (t, J = 7.1 Hz, 1H), 7.30 (t, J = 7.9 Hz, 1H), 6.26 (s, 1H), |
| | | 2.84-2.70 (m, 1H), 2.61-2.53 (m, 1H), 2.11-2.02 (m, 3H), 1.99- |
| | | 1.84 (m, 5H), 1.41 (s, 3H), 1.09 (q, J = 3.6 Hz, 2H), 0.66 (q, J = 3.7 Hz, 2H). |
| 4.13 | 588.2 | 1H NMR (400 MHz, DMSO-d6) δ 9.12 (s, 1H), 8.81 (s, 1H), 7.77 (t, J = |
| | | 7.1 Hz, 1H), 7.68 (d, J = 4.9 Hz, 1H), 7.55 (t, J = 7.2 Hz, 1H), 7.29 (t, |
| | | J = 7.9 Hz, 1H), 6.14 (s, 1H), 5.98 (d, J = 4.4 Hz, 1H), 4.75 (s, 2H), |
| | | 4.07-3.86 (m, 4H), 2.47-2.37 (m, 2H), 2.08-2.00 (m, 3H), 1.95- |
| | | 1.89 (m, 3H), 1.59 (s, 2H), 1.40 (s, 3H), 1.09 (q, J = 3.7 Hz, 2H), 0.66 |
| | | (q, J = 3.7 Hz, 2H). |
| 4.14 | 573.2 | 1H NMR (400 MHz, DMSO-d6) δ 9.12 (s, 1H), 8.82 (s, 1H), 7.86- |
| | | 7.73 (m, 1H), 7.73-7.59 (m, 2H), 7.59-7.49 (m, 1H), 7.42-7.34 (m, |
| | | 1H), 7.30 (t, J = 7.9 Hz, 1H), 6.37 (d, J = 9.3 Hz, 1H), 6.15 (s, 1H), 3.41 |
| | | (s, 3H), 2.39-2.19 (m, 2H), 2.10-1.99 (m, 3H), 1.92 (s, 3H), 1.73- |
| | | 1.50 (m, 2H), 1.40 (s, 3H), 1.09 (q, J = 3.7 Hz, 2H), 0.66 (q, J = 3.7 Hz, 2H). |
| 4.15 | 578.1 | 1H NMR (400 MHz, DMSO-d6) δ 9.12 (s, 1H), 8.85-8.75 (m, 2H), |
| | | 8.63 (s, 1H), 7.83-7.73 (m, 1H), 7.71-7.64 (m, 1H), 7.55 (td, J = 7.6, |
| | | 1.9 Hz, 1H), 7.30 (t, J = 7.9 Hz, 1H), 6.27 (s, 1H), 2.90-2.71 (m, 2H), |
| | | 2.07 (d, J = 6.1 Hz, 3H), 1.94 (s, 3H), 1.88-1.68 (m, 2H), 1.41 (s, 3H), |
| | | 1.09 (q, J = 3.6 Hz, 2H), 0.66 (q, J = 3.7 Hz, 2H). |
| 4.16 | 565.2 | 1H NMR (400 MHz, DMSO-d6) δ 8.84 (s, 1H), 8.38 (d, J = 2.3 Hz, |
| | | 1H), 8.01 (td, J = 7.6, 2.2 Hz, 1H), 7.79 (d, J = 1.9 Hz, 1H), 7.69 (d, J = |
| | | 2.3 Hz, 1H), 7.50-7.38 (m, 2H), 6.27 (s, 1H), 3.02 (s, 3H), 2.88 (s, |
| | | 3H), 2.56-2.43 (m, 5H), 2.08 (s, 3H), 1.95 (s, 3H), 1.87-1.64 (m, 2H). |
| 4.17 | 549.2 | 1H NMR (400 MHz, DMSO-d6) δ 8.83 (s, 1H), 8.36-8.32 (m, 1H), |
| | | 8.01 (td, J = 7.6, 2.1 Hz, 1H), 7.78 (d, J = 1.9 Hz, 1H), 7.61-7.52 (m, |
| | | 1H), 7.50-7.36 (m, 2H), 6.22 (s, 1H), 3.02 (s, 3H), 2.88 (s, 3H), 2.50 |
| | | (p, J = 1.8 Hz, 2H), 2.07 (s, 3H), 1.94 (s, 3H), 1.82-1.68 (m, 2H). |
| | | (Additional CH3 under DMSO solvent peak) |
| 4.18 | 573.1 | 1H NMR (400 MHz, DMSO-d6) δ 9.42 (s, 1H), 8.83 (s, 1H), 8.07- |
| | | 7.94 (m, 2H), 7.79 (s, 1H), 7.50-7.38 (m, 3H), 7.38-7.29 (m, 1H), |
| | | 6.32 (s, 1H), 3.29 (dt, J = 9.5, 5.6 Hz, 1H), 3.02 (s, 3H), 2.92-2.79 (m, |
| | | 4H), 2.08 (s, 3H), 2.07-2.01 (m, 1H), 1.98 (s, 3H), 1.83 (dt, J = 10.5, |
| | | 5.4 Hz, 1H). |
| 4.19 | 573.1 | 1H NMR (400 MHz, DMSO-d6) δ 9.42 (s, 1H), 8.84 (s, 1H), 8.01 (d, J = |
| | | 7.9 Hz, 2H), 7.80 (s, 1H), 7.56 (t, J = 7.8 Hz, 1H), 7.50-7.27 (m, |
| | | 3H), 6.32 (s, 1H), 3.02 (s, 3H), 2.89 (s, 3H), 2.80-2.71 (m, 1H), 2.71- |
| | | 2.59 (m, 1H), 2.10 (s, 3H), 1.97 (s, 3H), 1.87 (dt, J = 8.8, 5.5 Hz, 1H), |
| | | 1.77 (dt, J = 10.6, 5.3 Hz, 1H). |
| 4.20 | 549.1 | 1H NMR (400 MHz, DMSO-d6) δ 8.83 (s, 1H), 8.01 (td, J = 7.6, 2.1 |
| | | Hz, 1H), 7.80-7.74 (m, 1H), 7.59-7.51 (m, 1H), 7.50-7.38 (m, 2H), |
| | | 7.37-7.27 (m, 1H), 6.20 (s, 1H), 3.01 (d, J = 1.9 Hz, 3H), 2.88 (s, 3H), |
| | | 2.77-2.63 (m, 2H), 2.43 (d, J = 3.0 Hz, 3H), 2.10-2.02 (m, 3H), 1.93 |
| | | (s, 3H), 1.81-1.58 (m, 2H). |
| 4.21 | 521.1 | 1H NMR (400 MHz, DMSO-d6) δ 8.82 (s, 1H), 8.00 (td, J = 7.6, 2.2 |
| | | Hz, 1H), 7.87 (s, 1H), 7.77 (d, J = 1.9 Hz, 1H), 7.49-7.37 (m, 2H), |
| | | 6.15 (s, 1H), 3.01 (s, 3H), 2.88 (s, 3H), 2.59-2.42 (m, 2H), 2.38 (s, |
| | | 3H), 2.06 (s, 3H), 1.91 (s, 3H), 1.64-1.49 (m, 2H). |
| 4.22 | 538.1 | 1H NMR (400 MHz, DMSO-d6) δ 8.82 (s, 1H), 8.00 (t, J = 7.6 Hz, |
| | | 1H), 7.80-7.74 (m, 2H), 7.49-7.38 (m, 2H), 6.19 (s, 1H), 3.72 (s, |
| | | 3H), 3.01 (d, J = 1.2 Hz, 3H), 2.88 (s, 3H), 2.64-2.58 (m, 1H), 2.48- |
| | | 2.35 (m, 1H), 2.10-2.02 (m, 3H), 1.92 (s, 3H), 1.71-1.47 (m, 2H). |
| 4.23 | 578.1 | 1H NMR (400 MHz, DMSO-d6) δ 9.27 (d, J = 2.3 Hz, 1H), 9.12 (s, |
| | | 1H), 8.82 (s, 1H), 8.11-7.98 (m, 1H), 7.82-7.73 (m, 1H), 7.73-7.64 |
| | | (m, 1H), 7.55 (t, J = 7.1 Hz, 1H), 7.30 (t, J = 7.9 Hz, 1H), 6.28 (s, 1H), |
| | | 3.03-2.91 (m, 1H), 2.91-2.78 (m, 1H), 2.14-1.83 (m, 8H), 1.46- |
| | | 1.31 (m, 3H), 1.10 (q, J = 3.6 Hz, 2H), 0.66 (q, J = 3.8 Hz, 2H). |
| 4.24 | 585.1 | 1H NMR (400 MHz, DMSO-d6) δ 8.84 (s, 1H), 8.55 (d, J = 2.6 Hz, |
| | | 1H), 8.01 (td, J = 7.6, 2.2 Hz, 1H), 7.84-7.75 (m, 2H), 7.49-7.37 (m, |
| | | 2H), 7.16 (t, J = 53.8 Hz, 1H), 6.30-6.21 (m, 1H), 3.02 (s, 3H), 2.88 |
| | | (s, 3H), 2.83-2.72 (m, 1H), 2.68-2.59 (m, 1H), 2.06 (s, 3H), 1.97- |
| | | 1.94 (m, 3H), 1.88 (dt, J = 8.8, 5.8 Hz, 1H), 1.78 (dt, J = 8.9, 5.7 Hz, 1H). |
| 4.25 | 535.1 | 1H NMR (400 MHz, DMSO-d6) δ 8.83 (s, 1H), 8.49 (t, J = 1.8 Hz, |
| | | 1H), 8.43 (d, J = 2.7 Hz, 1H), 8.01 (td, J = 7.6, 2.2 Hz, 1H), 7.79 (d, J = |
| | | 1.9 Hz, 1H), 7.66 (dt, J = 10.4, 2.3 Hz, 1H), 7.50-7.34 (m, 2H), 6.24 |
| | | (s, 1H), 3.02 (s, 3H), 2.88 (s, 3H), 2.63-2.57 (m, 2H), 2.07 (s, 3H), |
| | | 1.94 (s, 3H), 1.86-1.71 (m, 2H). |
| 4.26 | 560.1 | 1H NMR (400 MHz, DMSO-d6) δ 8.83 (s, 1H), 8.67 (d, J = 2.6 Hz, |
| | | 1H), 8.01 (td, J = 7.6, 2.1 Hz, 1H), 7.95 (dd, J = 9.7, 2.6 Hz, 1H), 7.79 |
| | | (d, J = 1.9 Hz, 1H), 7.48-7.37 (m, 2H), 6.34 (s, 1H), 3.02 (s, 3H), 2.88 |
| | | (s, 3H), 2.81-2.63 (m, 2H), 2.07 (s, 3H), 1.96 (s, 3H), 1.93-1.84 (m, 2H). |
| 4.27 | 556.0 | 1H NMR (400 MHz, DMSO-d6) δ 8.83 (s, 1H), 8.15 (d, J = 2.6 Hz, |
| | | 1H), 8.05-7.96 (m, 1H), 7.89-7.75 (m, 1H), 7.73 (t, J = 59.2 Hz, |
| | | 1H), 7.50-7.37 (m, 2H), 6.52-6.46 (m, 1H), 6.19 (s, 1H), 3.01 (s, |
| | | 3H), 2.88 (s, 3H), 2.58 (td, J = 6.7, 3.4 Hz, 2H), 2.06 (s, 3H), 1.93 (s, |
| | | 3H), 1.73-1.58 (m, 2H). |
| 4.28 | 566.1 | 1H NMR (400 MHz, DMSO-d6) δ 8.83 (s, 1H), 8.01 (t, J = 7.3 Hz, |
| | | 1H), 7.78 (d, J = 1.9 Hz, 1H), 7.45 (m, 6H), 7.01 (t, J = 55.9 Hz, 1H), |
| | | 6.23 (s, 1H), 3.02 (s, 3H), 2.88 (s, 3H), 2.65-2.58 (m, 1H), 2.55-2.45 |
| | | (m, 1H), 2.07 (s, 3H), 1.94 (s, 3H), 1.74 (m, 2H). |
| 4.29 | 578.1 | 1H NMR (400 MHz, DMSO-d6) δ 9.12 (s, 1H), 8.87-8.75 (m, 1H), |
| | | 8.69-8.58 (m, 1H), 7.78 (t, J = 7.5 Hz, 1H), 7.72-7.62 (m, 2H), 7.59- |
| | | 7.49 (m, 1H), 7.30 (t, J = 7.9 Hz, 1H), 6.35-6.19 (m, 1H), 2.91- |
| | | 2.70 (m, 2H), 2.12-2.00 (m, 3H), 1.97-1.80 (m, 5H), 1.44-1.37 (m, |
| | | 3H), 1.09 (q, J = 3.4 Hz, 2H), 0.66 (q, J = 3.8 Hz, 2H). |
| 4.30 | 578.1 | 1H NMR (400 MHz, DMSO-d6) δ 9.12 (s, 1H), 9.05-8.90 (m, 1H), |
| | | 8.82 (s, 1H), 7.88-7.82 (m, 1H), 7.78 (t, J = 7.1 Hz, 1H), 7.73-7.63 |
| | | (m, 1H), 7.55 (t, J = 7.5 Hz, 1H), 7.30 (t, J = 7.9 Hz, 1H), 6.26 (s, 1H), |
| | | 2.97-2.88 (m, 1H), 2.83-2.68 (m, 1H), 2.09-2.01 (m, 3H), 1.96- |
| | | 1.82 (m, 5H), 1.41 (s, 3H), 1.12-1.04 (m, 2H), 0.66 (q, J = 3.7 Hz, 2H). |
| 4.31 | 582.2 | 1H NMR (400 MHz, DMSO-d6) δ 9.12 (s, 1H), 8.82 (s, 1H), 8.67- |
| | | 8.56 (m, 1H), 8.02-7.89 (m, 1H), 7.82-7.67 (m, 3H), 7.55 (td, J = |
| | | 7.7, 7.2, 1.9 Hz, 1H), 7.30 (t, J = 7.9 Hz, 1H), 7.27-7.12 (m, 1H), 6.92- |
| | | 6.80 (m, 1H), 6.23 (s, 1H), 2.66-2.58 (m, 1H), 2.12-2.04 (m, 3H), |
| | | 2.00-1.87 (m, 4H), 1.78-1.63 (m, 2H), 1.41 (s, 3H), 1.10 (q, J = 3.7 |
| | | Hz, 2H), 0.66 (q, J = 3.7 Hz, 2H). |
| 4.32 | 600.2 | 1H NMR (400 MHz, DMSO-d6) δ 9.12 (s, 1H), 8.81 (s, 1H), 7.85- |
| | | 7.73 (m, 1H), 7.73-7.63 (m, 1H), 7.58-7.49 (m, 1H), 7.42-7.20 (m, |
| | | 2H), 6.10 (s, 1H), 3.79 (s, 2H), 2.72-2.62 (m, 2H), 2.41-2.32 (m, |
| | | 1H), 2.26-2.19 (m, 1H), 2.10-2.02 (m, 3H), 1.94-1.85 (m, 3H), |
| | | 1.59-1.42 (m, 2H), 1.40 (s, 3H), 1.26-1.18 (m, 6H), 1.09 (q, J = 3.6 |
| | | Hz, 2H), 0.66 (q, J = 3.7 Hz, 2H). |
| 4.33 | 546.2 | 1H NMR (400 MHz, DMSO-d6) δ 9.12 (s, 1H), 8.81 (s, 1H), 7.82- |
| | | 7.71 (m, 1H), 7.71-7.65 (m, 1H), 7.65-7.50 (m, 2H), 7.42-7.22 (m, |
| | | 2H), 6.10 (s, 1H), 3.78 (s, 3H), 2.34-2.25 (m, 2H), 2.11-1.99 (m, |
| | | 3H), 1.97-1.82 (m, 3H), 1.62-1.45 (m, 2H), 1.40 (s, 3H), 1.09 (q, J = |
| | | 3.6 Hz, 2H), 0.66 (q, J = 3.7 Hz, 2H). |
| 4.34 | 549.1 | 1H NMR (400 MHz, DMSO-d6) δ 9.12 (s, 1H), 9.07-8.98 (m, 1H), |
| | | 8.81 (s, 1H), 7.83-7.73 (m, 1H), 7.73-7.62 (m, 1H), 7.61-7.48 (m, |
| | | 1H), 7.43-7.36 (m, 1H), 7.30 (t, J = 7.9 Hz, 1H), 6.20 (s, 1H), 2.93- |
| | | 2.71 (m, 2H), 2.11-2.00 (m, 3H), 1.93 (s, 3H), 1.88-1.65 (m, 2H), |
| | | 1.48-1.35 (m, 3H), 1.09 (q, J = 3.5 Hz, 2H), 0.70-0.63 (m, 2H). |
| 4.35 | 634.1 | 1H NMR (400 MHz, DMSO-d6) δ 9.13 (s, 1H), 8.82 (s, 1H), 8.34- |
| | | 8.20 (m, 1H), 8.18 (d, J = 2.3 Hz, 1H), 7.81-7.73 (m, 1H), 7.73-7.60 |
| | | (m, 2H), 7.60-7.44 (m, 1H), 7.30 (t, J = 7.9 Hz, 1H), 6.23 (s, 1H), |
| | | 3.40 (d, J = 1.2 Hz, 6H), 2.68-2.58 (m, 2H), 2.13-2.02 (m, 3H), 1.94 |
| | | (s, 3H), 1.88-1.71 (m, 2H), 1.41 (s, 3H), 1.09 (q, J = 3.6 Hz, 2H), 0.66 |
| | | (q, J = 3.7 Hz, 2H). |
| 4.36 | 549.1 | 1H NMR (400 MHz, DMSO-d6) δ 9.12 (s, 1H), 8.91-8.74 (m, 2H), |
| | | 8.67-8.52 (m, 1H), 7.82-7.73 (m, 1H), 7.73-7.62 (m, 1H), 7.62- |
| | | 7.50 (m, 1H), 7.30 (t, J = 7.9 Hz, 1H), 6.27-6.13 (m, 1H), 2.74-2.52 |
| | | (m, 2H), 2.16-2.00 (m, 3H), 1.99-1.85 (m, 3H), 1.84-1.62 (m, 2H), |
| | | 1.41 (s, 3H), 1.15-1.03 (m, 2H), 0.66 (q, J = 3.7 Hz, 2H). |
| 4.37 | 546.8 | 1H NMR (400 MHz, Acetonitrile-d3) δ 8.67 (s, 1H), 7.99-7.80 (m, |
| | | 2H), 7.68-7.56 (m, 1H), 7.52 (s, 2H), 7.18 (t, J = 7.9 Hz, 1H), 6.02- |
| | | 5.70 (m, 1H), 3.96-3.82 (m, 3H), 2.70-2.49 (m, 1H), 2.40-2.27 (m, |
| | | 1H), 2.01 (s, 3H), 1.84-1.82 (m, 3H), 1.72-1.58 (m, 1H), 1.58-1.38 |
| | | (m, 1H), 1.36 (s, 3H), 1.08 (q, J = 3.8 Hz, 2H), 0.62 (q, J = 3.8 Hz, 2H). |
| 4.38 | 548.2 | 1H NMR (400 MHz, DMSO-d6) δ 9.12 (s, 1H), 8.82 (s, 1H), 7.78 (t, J = |
| | | 7.2 Hz, 1H), 7.74-7.67 (m, 1H), 7.64-7.49 (m, 1H), 7.30 (t, J = 7.9 |
| | | Hz, 1H), 6.27 (s, 1H), 4.23 (s, 3H), 2.89-2.73 (m, 2H), 2.06 (s, 3H), |
| | | 1.93 (s, 3H), 1.84-1.70 (m, 2H), 1.45-1.33 (m, 3H), 1.12-1.01 (m, |
| | | 2H), 0.69-0.57 (m, 2H). |
| 4.39 | 549.1 | 1H NMR (400 MHz, DMSO-d6) δ 9.12 (s, 1H), 8.87-8.76 (m, 1H), |
| | | 8.46 (d, J = 1.7 Hz, 1H), 7.78 (td, J = 7.5, 1.8 Hz, 1H), 7.73-7.61 (m, |
| | | 1H), 7.55 (td, J = 7.5, 1.8 Hz, 1H), 7.37-7.21 (m, 2H), 6.32-6.19 (m, |
| | | 1H), 3.02-2.81 (m, 1H), 2.67-2.59 (m, 1H), 2.07 (s, 3H), 1.96-1.84 |
| | | (m, 4H), 1.80-1.69 (m, 1H), 1.41 (s, 3H), 1.09 (q, J = 3.7 Hz, 2H), |
| | | 0.66 (q, J = 3.7 Hz, 2H). |
| 4.40 | 547.1 | 1H NMR (400 MHz, DMSO-d6) δ 9.12 (s, 1H), 8.81 (s, 1H), 7.82- |
| | | 7.72 (m, 1H), 7.72-7.63 (m, 2H), 7.58-7.50 (m, 1H), 7.30 (t, J = 7.9 |
| | | Hz, 1H), 6.18 (s, 1H), 4.10 (s, 3H), 2.62-2.53 (m, 2H), 2.06 (s, 3H), |
| | | 1.92 (s, 3H), 1.77-1.54 (m, 2H), 1.40 (s, 3H), 1.13-1.00 (m, 2H), |
| | | 0.66 (q, J = 3.7 Hz, 2H). |
| 4.41 | 550.1 | 1H NMR (400 MHz, DMSO-d6) δ 9.12 (s, 1H), 8.94-8.86 (m, 1H), |
| | | 8.86-8.76 (m, 1H), 7.78 (td, J = 7.5, 1.8 Hz, 1H), 7.72-7.65 (m, 1H), |
| | | 7.55 (t, J = 7.5 Hz, 1H), 7.30 (t, J = 7.9 Hz, 1H), 6.26 (s, 1H), 3.15- |
| | | 2.96 (m, 1H), 2.89-2.76 (m, 1H), 2.12-2.00 (m, 3H), 1.94 (s, 3H), |
| | | 1.91-1.76 (m, 2H), 1.50-1.32 (m, 3H), 1.13-1.05 (m, 2H), 0.76- |
| | | 0.59 (m, 2H). |
| 4.42 | 586.2 | 1H NMR (400 MHz, DMSO-d6) δ 9.12 (s, 1H), 8.81 (s, 1H), 7.77 (td, J = |
| | | 7.5, 1.8 Hz, 1H), 7.71-7.65 (m, 1H), 7.55 (t, J = 7.3 Hz, 1H), 7.29 (t, |
| | | J = 7.9 Hz, 1H), 6.12 (s, 1H), 3.62 (s, 3H), 2.67-2.44 (m, 7H), 2.38- |
| | | 2.23 (m, 1H), 2.08-2.00 (m, 3H), 1.91 (s, 3H), 1.63-1.47 (m, 2H), |
| | | 1.40 (s, 3H), 1.09 (q, J = 3.6 Hz, 2H), 0.74-0.59 (m, 2H). |
| 4.43 | 599.1 | 1H NMR (400 MHz, DMSO-d6) δ 9.42 (s, 1H), 9.12 (s, 1H), 8.83 (s, |
| | | 1H), 8.01 (d, J = 8.1 Hz, 1H), 7.78 (t, J = 7.3 Hz, 2H), 7.71 (s, 1H), 7.56 |
| | | (t, J = 7.8 Hz, 1H), 7.39 (d, J = 7.4 Hz, 1H), 7.30 (t, J = 7.9 Hz, 1H), |
| | | 6.32 (s, 1H), 2.73 (m, 1H), 2.70-2.60 (m, 1H), 2.10 (s, 3H), 1.97 (s, |
| | | 3H), 1.92-1.82 (m, 1H), 1.78 (dd, J = 9.1, 5.3 Hz, 1H), 1.41 (s, 3H), |
| | | 1.10 (q, J = 3.7 Hz, 2H), 0.66 (q, J = 3.7 Hz, 2H). |
| 4.44 | 597.2 | 1H NMR (400 MHz, DMSO-d6) δ 9.12 (s, 1H), 8.82 (s, 1H), 8.59 (s, |
| | | 1H), 7.78 (t, J = 7.1 Hz, 1H), 7.70 (s, 1H), 7.61-7.52 (m, 2H), 7.48 (t, |
| | | J = 7.5 Hz, 1H), 7.40 (d, J = 7.6 Hz, 1H), 7.30 (t, J = 7.9 Hz, 1H), 6.25 |
| | | (s, 1H), 4.49 (d, J = 17.7 Hz, 1H), 4.42 (d, J = 17.5 Hz, 1H), 2.61-2.48 |
| | | (m, 2H), 2.08 (s, 3H), 1.95 (s, 3H), 1.84-1.62 (m, 2H), 1.41 (s, 3H), |
| | | 1.09 (q, J = 3.7 Hz, 2H), 0.66 (q, J = 3.7 Hz, 2H). |
| 4.45 | 559.1 | 1H NMR (400 MHz, DMSO-d6) δ 11.63 (s, 1H), 9.12 (s, 1H), 8.81 (s, |
| | | 1H), 7.86-7.73 (m, 1H), 7.68 (s, 1H), 7.55 (t, J = 7.1 Hz, 1H), 7.37- |
| | | 7.13 (m, 3H), 6.17 (d, J = 2.7 Hz, 2H), 2.55-2.45 (m, 2H), 2.07 (s, 3H), |
| | | 1.93 (s, 3H), 1.76 (q, J = 6.6 Hz, 1H), 1.53 (dt, J = 9.8, 5.2 Hz, 1H), |
| | | 1.41 (s, 3H), 1.09 (q, J = 3.6 Hz, 2H), 0.66 (q, J = 3.8 Hz, 2H). |
| 4.46 | 563.1 | 1H NMR (400 MHz, DMSO-d6) δ 9.12 (s, 1H), 8.81 (s, 1H), 7.77 (t, J = |
| | | 7.4 Hz, 1H), 7.73-7.60 (m, 1H), 7.55 (t, J = 7.5 Hz, 1H), 7.40-7.22 |
| | | (m, 2H), 6.21-6.03 (m, 1H), 2.77-2.57 (m, 5H), 2.10-2.02 (m, 3H), |
| | | 1.97-1.88 (m, 3H), 1.79-1.54 (m, 2H), 1.44-1.32 (m, 3H), 1.13- |
| | | 1.02 (m, 2H), 0.71-0.59 (m, 2H). |
| 4.47 | 597.1 | 1H NMR (400 MHz, DMSO-d6) δ 10.35 (s, 1H), 9.12 (s, 1H), 8.81 (s, |
| | | 1H), 7.78 (t, J = 7.0 Hz, 1H), 7.68 (s, 1H), 7.55 (t, J = 7.3 Hz, 1H), 7.30 |
| | | (t, J = 7.9 Hz, 1H), 7.13 (d, J = 7.7 Hz, 1H), 6.84 (d, J = 7.1 Hz, 1H), |
| | | 6.67 (s, 1H), 6.18 (s, 1H), 3.44 (s, 2H), 2.55-2.45 (m, 2H), 2.07 (s, |
| | | 3H), 1.93 (s, 3H), 1.65 (m, 2H), 1.41 (s, 3H), 1.09 (q, J = 3.8 Hz, 2H), |
| | | 0.66 (q, J = 3.8 Hz, 2H). |
| 4.48 | 597.2 | 1H NMR (400 MHz, DMSO-d6) δ 10.40 (s, 1H), 9.12 (s, 1H), 8.82 (s, |
| | | 1H), 7.78 (t, J = 7.4 Hz, 1H), 7.69 (s, 1H), 7.55 (t, J = 7.7 Hz, 1H), 7.30 |
| | | (t, J = 7.9 Hz, 1H), 7.16 (t, J = 7.8 Hz, 1H), 6.72 (d, J = 8.0 Hz, 1H), |
| | | 6.70 (d, J = 7.9 Hz, 1H), 6.21 (s, 1H), 3.52 (d, J = 4.1 Hz, 2H), 2.55- |
| | | 2.45 (m, 2H), 2.08 (s, 3H), 1.94 (s, 3H), 1.73-1.55 (m, 2H), 1.41 (s, |
| | | 3H), 1.09 (m, 2H), 0.66 (m, 2H). |
| 4.49 | 552.5 | 1H NMR (400 MHz, Chloroform-d) δ 8.83 (s, 1H), 8.35 (d, J = 5.2 Hz, |
| | | 1H), 8.08-7.98 (m, 1H), 7.57 (s, 1H), 7.44 (ddd, J = 7.8, 6.2, 1.9 Hz, |
| | | 1H), 7.36 (t, J = 7.6 Hz, 1H), 7.19 (d, J = 1.5 Hz, 1H), 7.06 (dd, J = 5.3, |
| | | 1.6 Hz, 1H), 5.84-5.77 (m, 1H), 3.16 (s, 3H), 2.97 (d, J = 1.2 Hz, 3H), |
| | | 2.75-2.66 (m, 1H), 2.32-2.26 (m, 1H), 2.20 (s, 3H), 1.99 (s, 3H), |
| | | 1.74-1.64 (m, 2H). |
| 4.50 | 551.1 | 1H NMR (400 MHz, Chloroform-d) δ 8.85 (s, 1H), 8.42-8.32 (m, |
| | | 1H), 8.06-7.93 (m, 1H), 7.57 (d, J = 1.7 Hz, 1H), 7.54-7.41 (m, 2H), |
| | | 7.41-7.29 (m, 2H), 5.85-5.77 (m, 1H), 3.16 (s, 3H), 2.97 (s, 3H), |
| | | 2.67-2.58 (m, 1H), 2.40-2.29 (m, 1H), 2.22 (s, 3H), 1.99 (s, 3H), |
| | | 1.69-1.58 (m, 2H). |
| 4.51 | 594.0 | 1H NMR (400 MHz, DMSO-d6) δ 13.19 (s, 1H), 9.12 (s, 1H), 8.81 (s, |
| | | 1H), 7.78 (t, J = 7.4 Hz, 1H), 7.69 (s, 1H), 7.55 (t, J = 7.2 Hz, 1H), 7.39 |
| | | (s, 1H), 7.30 (t, J = 7.9 Hz, 1H), 6.22 (s, 1H), 2.85-2.68 (m, 1H), 2.63- |
| | | 2.56 (m, 1H), 2.06 (s, 3H), 2.02-1.88 (m, 4H), 1.72 (dd, J = 9.8, 5.2 |
| | | Hz, 1H), 1.40 (s, 3H), 1.09 (q, J = 3.6 Hz, 2H), 0.66 (q, J = 3.7 Hz, 2H). |
| 4.52 | 567.2 | 1H NMR (400 MHz, Chloroform-d) δ 8.83 (s, 1H), 8.61 (d, J = 5.2 Hz, |
| | | 1H), 8.04 (s, 1H), 7.57 (s, 1H), 7.53-7.49 (m, 1H), 7.44 (ddd, J = 7.8, |
| | | 6.0, 1.9 Hz, 1H), 7.36 (t, J = 7.7 Hz, 1H), 7.28-7.27 (m, 1H), 6.70 (t, J = |
| | | 55.3 Hz, 1H), 5.83 (d, J = 1.0 Hz, 1H), 3.16 (s, 3H), 2.97 (s, 3H), 2.78- |
| | | 2.71 (m, 1H), 2.43-2.34 (m, 1H), 2.20 (s, 3H), 1.99 (s, 3H), 1.79- |
| | | 1.66 (m, 2H). |
| 4.53 | 547.3 | 1H NMR (400 MHz, Chloroform-d) δ 8.83 (s, 1H), 8.21 (d, J = 5.6 Hz, |
| | | 1H), 8.10-7.96 (m, 1H), 7.56 (s, 1H), 7.44 (ddd, J = 7.8, 6.1, 1.9 Hz, |
| | | 1H), 7.36 (t, J = 7.6 Hz, 1H), 6.80 (dd, J = 5.7, 1.7 Hz, 1H), 6.69-6.61 |
| | | (m, 1H), 5.86-5.76 (m, 1H), 4.01 (s, 3H), 3.15 (s, 3H), 2.97 (s, 3H), |
| | | 2.76-2.69 (m, 1H), 2.35-2.25 (m, 1H), 2.20 (s, 3H), 1.98 (s, 3H), |
| | | 1.74-1.61 (m, 2H). |
| 4.54 | 535.1 | 1H NMR (400 MHz, Chloroform-d) δ 8.83 (s, 1H), 8.21-8.13 (m, |
| | | 1H), 8.04 (s, 1H), 7.62 (td, J = 8.0, 2.6 Hz, 1H), 7.57 (s, 1H), 7.48- |
| | | 7.40 (m, 1H), 7.36 (t, J = 7.6 Hz, 1H), 6.94 (dd, J = 8.5, 2.9 Hz, 1H), |
| | | 5.84-5.75 (m, 1H), 3.15 (s, 3H), 2.97 (s, 3H), 2.66-2.58 (m, 1H), |
| | | 2.42-2.34 (m, 1H), 2.21 (s, 3H), 1.98 (s, 3H), 1.65-1.57 (m, 2H). |
| 4.55 | 535.2 | 1H NMR (400 MHz, Chloroform-d) δ 8.83 (s, 1H), 8.17 (d, J = 5.3 Hz, |
| | | 1H), 8.08-7.97 (m, 1H), 7.57 (s, 1H), 7.44 (ddd, J = 7.8, 6.2, 1.8 Hz, |
| | | 1H), 7.36 (t, J = 7.6 Hz, 1H), 7.04-6.94 (m, 1H), 6.77 (s, 1H), 5.81 (s, |
| | | 1H), 3.16 (s, 3H), 2.97 (s, 3H), 2.76-2.68 (m, 1H), 2.37-2.32 (m, |
| | | 1H), 2.20 (s, 3H), 1.99 (s, 3H), 1.74-1.64 (m, 2H). |
| 4.56 | 540.0 | 1H NMR (400 MHz, DMSO-d6) δ 11.63 (s, 1H), 8.86 (t, J = 0.7 Hz, |
| | | 1H), 8.32 (td, J = 7.6, 1.8 Hz, 1H), 7.96 (td, J = 7.2, 6.5, 1.9 Hz, 1H), |
| | | 7.88 (d, J = 1.9 Hz, 1H), 7.61 (t, J = 7.8 Hz, 1H), 7.25 (d, J = 6.8 Hz, |
| | | 2H), 6.19 (s, 1H), 6.16 (t, J = 6.7 Hz, 1H), 3.39 (s, 3H), 2.55-2.4 (m, |
| | | 2H), 2.10 (s, 3H), 1.95 (d, J = 0.9 Hz, 3H), 1.85-1.71 (m, 1H), 1.53 |
| | | (dt, J = 9.9, 5.2 Hz, 1H). |
| 4.57 | 553.9 | 1H NMR (400 MHz, DMSO-d6) δ 11.42 (s, 1H), 8.86 (s, 1H), 8.38- |
| | | 8.23 (m, 1H), 7.96 (t, J = 6.2 Hz, 1H), 7.87 (s, 1H), 7.61 (t, J = 7.8 Hz, |
| | | 1H), 7.13 (d, J = 2.5 Hz, 1H), 7.03 (s, 1H), 6.19 (s, 1H), 3.39 (s, 3H), |
| | | 2.55-2.45 (m, 2H), 2.10 (s, 3H), 2.01 (d, J = 1.1 Hz, 3H), 1.97-1.89 |
| | | (m, 3H), 1.83-1.71 (m, 1H), 1.52 (dt, J = 9.6, 5.0 Hz, 1H). |
| 4.58 | 558.0 | 1H NMR (400 MHz, DMSO-d6) δ 8.86 (s, 1H), 8.32 (t, J = 6.7 Hz, |
| | | 1H), 7.96 (t, J = 7.1 Hz, 1H), 7.88 (s, 1H), 7.61 (t, J = 7.8 Hz, 1H), 7.51- |
| | | 7.32 (m, 2H), 6.20 (s, 1H), 3.39 (s, 3H), 2.56-2.45 (m, 2H), 2.10 (s, |
| | | 3H), 1.95 (s, 3H), 1.88-1.76 (m, 1H), 1.64-1.52 (m, 1H). |
| 4.59 | 608.0 | 1H NMR (400 MHz, DMSO-d6) δ 12.26 (s, 1H), 8.86 (s, 1H), 8.32 (t, J = |
| | | 6.7 Hz, 1H), 8.01-7.92 (m, 1H), 7.88 (s, 1H), 7.82 (s, 1H), 7.61 (t, J = |
| | | 7.8 Hz, 1H), 7.49 (d, J = 2.6 Hz, 1H), 6.21 (s, 1H), 3.39 (s, 3H), 2.64- |
| | | 2.45 (m, 2H), 2.10 (s, 3H), 1.95 (m, 4H), 1.54 (dt, J = 10.2, 5.2 Hz, 1H). |
| 4.60 | 558.0 | 1H NMR (400 MHz, DMSO-d6) δ 11.64 (s, 1H), 8.78 (d, J = 0.7 Hz, |
| | | 1H), 8.07 (dt, J = 22.1, 6.9 Hz, 2H), 7.66 (t, J = 7.8 Hz, 1H), 7.25 (d, J = |
| | | 6.9 Hz, 2H), 6.26 (s, 1H), 6.16 (t, J = 6.6 Hz, 1H), 3.38 (s, 3H), 2.54- |
| | | 2.45 (m, 2H), 2.18 (s, 3H), 2.01 (s, 3H), 1.78 (m, 1H), 1.57 (dd, J = 9.8, |
| | | 5.0 Hz, 1H). |
| 4.61 | 558.0 | 1H NMR (400 MHz, DMSO-d6) δ 8.79 (d, J = 0.8 Hz, 1H), 8.10 (t, J = |
| | | 7.2 Hz, 1H), 8.05 (t, J = 7.3 Hz, 1H), 7.66 (t, J = 7.8 Hz, 1H), 7.40 (m, |
| | | 1H), 6.3-6.13 (m, 2H), 6.26 (s, 1H), 3.38 (s, 3H), 2.65-2.55 (m, 2H), |
| | | 2.20-2.13 (m, 3H), 2.01 (s, 3H), 1.85-1.76 (m, 1H), 1.74-1.61 (m, 1H). |
| 4.62 | 592.1 | 1H NMR (400 MHz, DMSO-d6) δ 8.79 (d, J = 0.8 Hz, 1H), 8.77-8.71 |
| | | (m, 1H), 8.68-8.61 (m, 1H), 8.15-8.00 (m, 2H), 7.90 (s, 1H), 7.67 (t, |
| | | J = 7.8 Hz, 1H), 7.14 (t, J = 55.3 Hz, 1H), 6.34 (s, 1H), 3.39 (s, 3H), |
| | | 2.73-2.56 (m, 2H), 2.18 (s, 3H), 2.03 (s, 3H), 1.96-1.72 (m, 2H). |
| 4.63 | 609.8 | 1H NMR (400 MHz, DMSO-d6) δ 8.89 (d, J = 2.0 Hz, 1H), 8.86-8.81 |
| | | (m, 1H), 8.79 (s, 1H), 8.16-8.00 (m, 3H), 7.67 (t, J = 7.8 Hz, 1H), |
| | | 6.35 (s, 1H), 3.39 (s, 3H), 2.75-2.63 (m, 2H), 2.18 (s, 3H), 2.04 (s, |
| | | 3H), 1.99-1.90 (m, 1H), 1.87-1.78 (m, 1H). |
| 4.64 | 574.8 | 1H NMR (400 MHz, DMSO-d6) δ 8.77 (s, 1H), 8.65 (d, J = 1.8 Hz, |
| | | 1H), 8.16-8.00 (m, 2H), 7.66 (t, J = 7.8 Hz, 1H), 6.34 (s, 1H), 3.38 (s, |
| | | 3H), 2.81 (dddd, J = 15.1, 8.7, 6.0, 4.2 Hz, 2H), 2.46 (d, J = 2.5 Hz, |
| | | 3H), 2.16 (s, 3H), 2.01 (s, 3H), 1.78 (dddd, J = 26.5, 8.7, 6.1, 4.4 Hz, 2H). |
| 4.65 | 566.1 | 1H NMR (400 MHz, DMSO-d6) δ 8.78 (s, 1H), 8.09 (td, J = 7.4, 6.6, |
| | | 1.7 Hz, 1H), 8.04 (ddd, J = 8.4, 6.7, 1.8 Hz, 1H), 7.66 (t, J = 7.8 Hz, |
| | | 1H), 7.36 (d, J = 3.3 Hz, 1H), 6.22 (s, 1H), 3.38 (s, 3H), 2.45-2.38 (m, |
| | | 1H), 2.26-2.19 (m, 1H), 2.18 (s, 3H), 1.99 (s, 3H), 1.60-1.51 (m, 2H). |
| 4.66 | 557.3 | 1H NMR (400 MHz, DMSO) δ 8.79 (s, 1H), 8.56 (s, 1H), 8.39 (s, 1H), |
| | | 8.11 (t, J = 7.2 Hz, 1H), 8.05 (t, J = 7.5 Hz, 1H), 7.67 (t, J = 7.8 Hz, |
| | | 1H), 6.35 (s, 1H), 3.39 (s, 3H), 2.80 (t, J = 7.3 Hz, 2H), 2.49 (s, 3H), |
| | | 2.18 (s, 3H), 2.03 (s, 3H), 1.86-1.73 (m, 2H). |
| 4.67 | 599.2 | 1H NMR (400 MHz, DMSO) δ 8.80 (s, 1H), 8.17 (d, J = 2.4 Hz, 1H), |
| | | 8.11 (t, J = 7.0 Hz, 1H), 8.05 (t, J = 7.1 Hz, 1H), 7.67 (t, J = 7.7 Hz, |
| | | 1H), 7.20-7.10 (m, 1H), 6.93-6.80 (m, 2H), 6.49 (s, 1H), 3.21 (d, J = |
| | | 9.8 Hz, 2H), 2.76-2.64 (m, 1H), 2.51 (t, J = 2.1 Hz, 3H), 2.21 (s, 3H), |
| | | 2.06 (s, 3H), 1.81 (dt, J = 10.5, 5.5 Hz, 1H). |
| 4.68 | 570.1 | 1H NMR (400 MHz, Chloroform-d) δ 8.61 (d, J = 0.8 Hz, 1H), 8.13- |
| | (M + Na)+ | 8.05 (m, 1H), 8.02-7.94 (m, 1H), 7.50 (t, J = 7.8 Hz, 1H), 7.31 (d, J = |
| | | 2.3 Hz, 1H), 6.07 (d, J = 2.3 Hz, 1H), 5.79 (s, 1H), 3.25 (s, 3H), 2.71- |
| | | 2.62 (m, 1H), 2.55-2.45 (m, 1H), 2.24 (s, 3H), 2.02 (s, 3H), 1.70- |
| | | 1.65 (m, 1H), 1.50 (dt, J = 9.1, 5.6 Hz, 1H). |
| 4.69 | 543.1 | 1H NMR (400 MHz, DMSO-d6) δ 9.08 (s, 1H), 8.78 (s, 1H), 8.67 (d, J = |
| | | 5.2 Hz, 1H), 8.14-7.94 (m, 2H), 7.76-7.48 (m, 2H), 6.34 (s, 1H), |
| | | 3.38 (s, 3H), 2.96-2.85 (m, 1H), 2.80-2.70 (m, 1H), 2.16 (s, 3H), |
| | | 2.02 (s, 3H), 1.91-1.77 (m, 2H). |
| 4.70 | 560.1 | 1H NMR (400 MHz, DMSO-d6) δ 8.78 (d, J = 0.8 Hz, 1H), 8.13- |
| | | 8.07 (m, 1H), 8.04 (ddd, J = 8.4, 6.8, 1.8 Hz, 1H), 7.66 (t, J = 7.8 Hz, |
| | | 1H), 6.33-6.23 (m, 1H), 4.01 (s, 3H), 3.38 (s, 3H), 2.63 (ddd, J = 8.7, |
| | | 5.8, 4.5 Hz, 1H), 2.50 (m, 1H), 2.25 (s, 3H), 2.22-2.13 (m, 3H), 2.01 |
| | | (s, 3H), 1.69-1.52 (m, 2H). |
| 4.71 | 561.1 | 1H NMR (400 MHz, DMSO-d6) δ 8.84 (s, 2H), 8.78 (s, 1H), 8.17- |
| | | 7.98 (m, 2H), 7.67 (t, J = 7.8 Hz, 1H), 6.36 (s, 1H), 3.39 (s, 3H), 2.95- |
| | | 2.80 (m, 2H), 2.17 (s, 3H), 2.02 (s, 3H), 1.90-1.69 (m, 2H). |
| 4.72 | 560.1 | 1H NMR (400 MHz, DMSO-d6) δ 8.78 (s, 1H), 8.57 (d, J = 5.1 Hz, |
| | | 1H), 8.15-7.99 (m, 2H), 7.66 (t, J = 7.8 Hz, 1H), 7.24 (d, J = 5.1 Hz, |
| | | 1H), 6.34 (s, 1H), 3.39 (s, 3H), 2.92-2.82 (m, 1H), 2.79-2.70 (m, |
| | | 1H), 2.17 (s, 3H), 2.02 (s, 3H), 1.89-1.79 (m, 1H), 1.79-1.67 (m, 1H). |
| 4.73 | 543.1 | 1H NMR (400 MHz, DMSO-d6) δ 8.99 (s, 1H), 8.72 (s, 3H), 8.10- |
| | | 7.90 (m, 2H), 7.60 (t, J = 7.8 Hz, 1H), 6.27 (s, 1H), 3.32 (s, 3H), 2.64- |
| | | 2.53 (m, 1H), 2.52-2.45 (m, 1H), 2.11 (s, 3H), 1.96 (s, 3H), 1.85- |
| | | 1.78 (m, 1H), 1.78-1.61 (m, 1H). |
| 4.74 | 569.7 | 1H NMR (400 MHz, Methanol-d4) δ 8.72 (d, J = 0.8 Hz, 1H), 8.16- |
| | | 8.04 (m, 2H), 7.63 (t, J = 7.8 Hz, 1H), 6.87 (s, 1H), 6.24 (s, 1H), 4.02 |
| | | (s, 3H), 3.31 (s, 3H), 2.80-2.71 (m, 1H), 2.58-2.50 (m, 1H), 2.24 (s, |
| | | 3H), 2.10 (s, 3H), 1.77 (dt, J = 8.5, 5.5 Hz, 1H), 1.70 (dt, J = 9.1, 5.5 |
| | | Hz, 1H). |
| 4.75 | 558.7 | 1H NMR (400 MHz, Methanol-d4) δ 8.72 (d, J = 0.8 Hz, 1H), 8.16- |
| | | 8.04 (m, 2H), 7.63 (t, J = 7.8 Hz, 1H), 6.22 (s, 1H), 6.00 (s, 1H), 3.76 |
| | | (s, 3H), 2.69-2.63 (m, 1H), 2.43 (dd, J = 13.5, 6.5 Hz, 1H), 2.30 (s, |
| | | 3H), 2.24 (d, J = 0.7 Hz, 3H), 2.10 (s, 3H), 1.73-1.63 (m, 2H), 1.31 (s, 3H). |
| 4.76 | 558.7 | 1H NMR (400 MHz, Methanol-d4) δ 8.71 (d, J = 0.8 Hz, 1H), 8.09 |
| | | (dddd, J = 22.2, 8.3, 6.6, 1.8 Hz, 2H), 7.62 (t, J = 7.8 Hz, 1H), 7.39 (s, |
| | | 1H), 6.22 (s, 1H), 3.81 (s, 3H), 3.30 (s, 3H), 2.50 (dt, J = 8.6, 5.1 Hz, |
| | | 1H), 2.35 (s, 3H), 2.25 (s, 4H), 2.09 (s, 3H), 1.65 (dt, J = 9.0, 5.3 Hz, |
| | | 1H), 1.59-1.52 (m, 1H). |
| 4.77 | 543.0 | 1H NMR (400 MHz, DMSO-d6) δ 9.28 (dd, J = 2.6, 1.2 Hz, 1H), 9.12 |
| | | (dd, J = 5.5, 1.1 Hz, 1H), 8.79 (s, 1H), 8.14-8.01 (m, 2H), 7.72-7.58 |
| | | (m, 2H), 6.35 (s, 1H), 3.38 (s, 3H), 2.72 (td, J = 7.7, 4.7 Hz, 1H), 2.58 |
| | | (td, J = 7.7, 4.6 Hz, 1H), 2.17 (d, J = 2.4 Hz, 3H), 2.02 (q, J = 4.6, 3.8 |
| | | Hz, 3H), 1.92-1.87 (m, 2H). |
| 4.78 | 556.0 | 1H NMR (400 MHz, DMSO-d6) δ 8.78 (s, 1H), 8.15-8.01 (m, 2H), |
| | | 7.81 (s, 1H), 7.66 (t, J = 7.8 Hz, 1H), 7.37 (d, J = 7.8 Hz, 1H), 7.26 (s, |
| | | 1H), 6.32 (s, 1H), 3.38 (s, 3H), 2.81 (ddd, J = 8.9, 6.1, 4.2 Hz, 1H), 2.72 |
| | | (dt, J = 9.3, 5.2 Hz, 1H), 2.16 (d, J = 2.4 Hz, 3H), 2.02 (s, 3H), 1.95- |
| | | 1.70 (m, 2H). |
| 4.79 | 561.1 | 1H NMR (400 MHz, DMSO-d6) δ 8.99 (d, J = 2.9 Hz, 1H), 8.88-8.66 |
| | | (m, 2H), 8.20-8.02 (m, 2H), 7.67 (t, J = 7.8 Hz, 1H), 6.44 (s, 1H), |
| | | 3.39 (s, 3H), 3.09-2.98 (m, 1H), 2.98-2.86 (m, 1H), 2.18 (s, 3H), |
| | | 2.03 (s, 3H), 1.95-1.82 (m, 2H). |
| 4.80 | 543.0 | 1H NMR (400 MHz, DMSO-d6) δ 9.10 (dd, J = 4.9, 1.6 Hz, 1H), 8.78 |
| | | (d, J = 0.8 Hz, 1H), 8.13-8.07 (m, 1H), 8.07-8.02 (m, 1H), 7.75 (dd, |
| | | J = 8.5, 1.7 Hz, 1H), 7.70-7.60 (m, 2H), 6.37 (s, 1H), 3.39 (s, 3H), |
| | | 3.00-2.92 (m, 1H), 2.89-2.80 (m, 1H), 2.17 (s, 3H), 2.03 (s, 3H), |
| | | 2.00-1.94 (m, 1H), 1.88-1.79 (m, 1H). |
| 4.81 | 571.1 | 1H NMR (400 MHz, DMSO-d6) δ 8.78 (s, 1H), 8.09 (t, J = 7.3 Hz, |
| | | 1H), 8.07-8.01 (m, 1H), 7.71 (s, 1H), 7.66 (t, J = 7.8 Hz, 1H), 7.37 (s, |
| | | 1H), 6.19 (s, 1H), 3.69-3.61 (m, 1H), 3.38 (s, 3H), 2.38-2.27 (m, |
| | | 2H), 2.17 (s, 3H), 1.99 (s, 3H), 1.62-1.52 (m, 2H), 1.03-0.97 (m, |
| | | 2H), 0.97-0.88 (m, 2H). |
| 4.82 | 563.0 | 1H NMR (400 MHz, DMSO-d6) δ 8.78 (d, J = 0.8 Hz, 1H), 8.12- |
| | | 8.07 (m, 1H), 8.07-8.01 (m, 1H), 7.66 (t, J = 7.8 Hz, 1H), 7.36 (d, J = |
| | | 3.3 Hz, 1H), 6.22 (s, 1H), 3.67 (s, 3H), 3.38 (s, 3H), 2.41 (ddd, J = 8.4, |
| | | 5.9, 4.4 Hz, 1H), 2.26-2.19 (m, 1H), 2.18 (s, 3H), 1.99 (s, 3H), 1.62- |
| | | 1.49 (m, 2H). |
| 4.83 | 572.1 | 1H NMR (400 MHz, DMSO-d6) δ 8.79 (d, J = 0.9 Hz, 1H), 8.10 (t, J = |
| | | 7.6 Hz, 1H), 8.05 (t, J = 7.3 Hz, 1H), 7.66 (t, J = 7.8 Hz, 1H), 7.37 (dd, |
| | | J = 9.1, 6.9 Hz, 1H), 6.36 (m, 2H), 6.22 (d, J = 6.9 Hz, 1H), 3.51 (s, |
| | | 3H), 3.38 (s, 3H), 2.70-2.61 (m, 1H), 2.61-2.53 (m, 1H), 2.18 (s, |
| | | 3H), 2.02 (s, 3H), 1.73 (m, 2H). |
| 4.84 | 582.0 | 1H NMR (400 MHz, DMSO-d6) δ 8.78 (d, J = 0.8 Hz, 1H), 8.66 (s, |
| | | 1H), 8.45-8.00 (m, 3H), 7.66 (t, J = 7.8 Hz, 1H), 6.31 (s, 1H), 3.38 (s, |
| | | 3H), 2.75-2.62 (m, 2H), 2.17 (s, 3H), 2.01 (s, 3H), 1.89-1.65 (m, 2H). |
| 4.85 | 582.0 | 1H NMR (400 MHz, DMSO-d6) δ 8.78 (d, J = 0.8 Hz, 1H), 8.24- |
| | | 7.92 (m, 4H), 7.66 (t, J = 7.8 Hz, 1H), 6.32 (s, 1H), 3.38 (s, 3H), 2.81- |
| | | 2.62 (m, 2H), 2.21-2.14 (m, 3H), 2.01 (s, 3H), 1.78 (t, J = 7.6 Hz, 2H). |
| 4.86 | 586.1 | 1H NMR (400 MHz, DMSO-d6) δ 8.78 (d, J = 0.8 Hz, 1H), 8.13- |
| | | 8.07 (m, 1H), 8.07-8.01 (m, 1H), 7.70 (s, 1H), 7.66 (t, J = 7.8 Hz, |
| | | 1H), 6.27 (s, 1H), 4.22 (d, J = 7.1 Hz, 2H), 3.38 (s, 3H), 2.66-2.56 (m, |
| | | 2H), 2.22-2.11 (m, 3H), 2.01 (s, 3H), 1.78-1.63 (m, 2H), 1.29 (qq, J = |
| | | 7.4, 3.6, 2.8 Hz, 1H), 0.60-0.48 (m, 2H), 0.37 (dt, J = 6.1, 4.6 Hz, 2H). |
| 4.87 | 574.1 | 1H NMR (400 MHz, DMSO-d6) δ 8.78 (d, J = 0.8 Hz, 1H), 8.13- |
| | | 8.07 (m, 1H), 8.07-8.01 (m, 1H), 7.71-7.62 (m, 2H), 6.30-6.22 (m, |
| | | 1H), 4.75 (hept, J = 6.7 Hz, 1H), 3.38 (s, 3H), 2.69-2.54 (m, 2H), 2.21- |
| | | 2.10 (m, 3H), 2.00 (s, 3H), 1.75-1.61 (m, 2H), 1.46 (dd, J = 6.6, 1.3 |
| | | Hz, 6H). |
| 4.88 | 574.1 | 1H NMR (400 MHz, DMSO-d6) δ 8.78 (d, J = 0.8 Hz, 1H), 8.10 (ddd, |
| | | J = 8.0, 6.7, 1.8 Hz, 1H), 8.04 (ddd, J = 8.2, 6.7, 1.8 Hz, 1H), 7.70 (s, |
| | | 1H), 7.66 (t, J = 7.8 Hz, 1H), 6.27 (s, 1H), 4.31 (t, J = 6.9 Hz, 2H), 3.38 |
| | | (s, 3H), 2.59 (dtd, J = 11.8, 7.6, 3.8 Hz, 2H), 2.21-2.13 (m, 3H), 2.00 |
| | | (s, 3H), 1.85 (h, J = 7.2 Hz, 2H), 1.69 (t, J = 7.5 Hz, 2H), 0.83 (t, J = |
| | | 7.4 Hz, 3H). |
| 4.89 | 560.0 | 1H NMR (400 MHz, DMSO-d6) δ 8.78 (d, J = 0.8 Hz, 1H), 8.10 (ddd, |
| | | J = 7.9, 6.7, 1.8 Hz, 1H), 8.04 (ddd, J = 8.3, 6.8, 1.8 Hz, 1H), 7.70 (s, |
| | | 1H), 7.66 (t, J = 7.8 Hz, 1H), 6.27 (d, J = 1.0 Hz, 1H), 4.38 (q, J = 7.3 |
| | | Hz, 2H), 3.38 (s, 3H), 2.67-2.54 (m, 2H), 2.21-2.12 (m, 3H), 2.01 (s, |
| | | 3H), 1.69 (ddd, J = 8.8, 6.5, 2.3 Hz, 2H), 1.43 (t, J = 7.3 Hz, 3H). |
| 4.90 | 562.0 | 1H NMR (400 MHz, DMSO-d6) δ 8.81-8.75 (m, 1H), 8.16-8.03 (m, |
| | | 2H), 7.67 (t, J = 7.8 Hz, 1H), 7.30 (s, 1H), 6.31-6.21 (m, 1H), 3.39 (s, |
| | | 3H), 2.78-2.66 (m, 2H), 2.64 (s, 3H), 2.24-2.14 (m, 3H), 2.01 (s, |
| | | 3H), 1.86-1.71 (m, 1H), 1.71-1.52 (m, 1H). |
| 4.91 | 548.0 | 1H NMR (400 MHz, DMSO-d6) δ 9.04 (d, J = 4.6 Hz, 1H), 8.81-8.77 |
| | | (m, 1H), 8.17-8.02 (m, 2H), 7.67 (t, J = 7.8 Hz, 1H), 7.41 (d, J = 4.7 |
| | | Hz, 1H), 6.37-6.17 (m, 1H), 3.39 (s, 3H), 2.98-2.84 (m, 1H), 2.84- |
| | | 2.69 (m, 1H), 2.22-2.13 (m, 3H), 2.02 (s, 3H), 1.89-1.80 (m, 1H), |
| | | 1.80-1.58 (m, 1H). |
| 4.92 | 571.0 | 1H NMR (400 MHz, DMSO-d6) δ 8.79 (d, J = 0.8 Hz, 1H), 8.14-8.07 |
| | | (m, 1H), 8.05 (td, J = 7.3, 6.6, 1.8 Hz, 1H), 7.67 (t, J = 7.8 Hz, 1H), |
| | | 7.29 (d, J = 1.8 Hz, 1H), 6.33 (s, 1H), 6.09 (d, J = 1.8 Hz, 1H), 3.64- |
| | | 3.54 (m, 1H), 3.39 (s, 3H), 2.67-2.60 (m, 1H), 2.48-2.40 (m, 1H), |
| | | 2.17 (s, 3H), 2.02 (s, 3H), 1.82-1.74 (m, 1H), 1.68 (dt, J = 8.8, 5.7 Hz, |
| | | 1H), 1.11-1.02 (m, 1H), 1.02-0.85 (m, 3H). |
| 4.93 | 563.1 | 1H NMR (400 MHz, DMSO-d6) δ 8.79 (d, J = 0.8 Hz, 1H), 8.13-8.07 |
| | | (m, 1H), 8.04 (td, J = 7.3, 6.7, 1.8 Hz, 1H), 7.66 (t, J = 7.8 Hz, 1H), |
| | | 6.32 (s, 1H), 5.85 (d, J = 5.7 Hz, 1H), 3.72 (d, J = 1.0 Hz, 3H), 3.38 (s, |
| | | 3H), 2.60-2.52 (m, 2H), 2.18 (s, 3H), 2.01 (s, 3H), 1.71 (dt, J = 9.0, |
| | | 5.4 Hz, 1H), 1.59 (dt, J = 8.9, 5.6 Hz, 1H). |
| 4.94 | 569.9 | 1H NMR (400 MHz, DMSO-d6) δ 8.78 (d, J = 0.8 Hz, 1H), 8.09 (td, J = |
| | | 7.7, 7.2, 1.8 Hz, 1H), 8.04 (td, J = 7.3, 6.6, 1.8 Hz, 1H), 7.79 (s, 1H), |
| | | 7.66 (t, J = 7.8 Hz, 1H), 7.57 (s, 1H), 6.21 (s, 1H), 5.43 (s, 2H), 3.38 (s, |
| | | 3H), 2.37 (t, J = 7.4 Hz, 2H), 2.17 (s, 3H), 2.00 (s, 3H), 1.65-1.51 (m, 2H). |
| 4.95 | 570.0 | 1H NMR (400 MHz, DMSO-d6) δ 8.78 (d, J = 0.8 Hz, 1H), 8.10 (td, J = |
| | | 7.3, 6.6, 1.8 Hz, 1H), 8.04 (ddd, J = 8.3, 6.7, 1.8 Hz, 1H), 7.96 (s, |
| | | 1H), 7.66 (t, J = 7.8 Hz, 1H), 6.30 (s, 1H), 3.91 (s, 3H), 3.38 (s, 3H), |
| | | 2.56-2.51 (m, 1H), 2.39 (dt, J = 10.5, 5.4 Hz, 1H), 2.17 (s, 3H), 2.01 |
| | | (s, 3H), 1.70 (dt, J = 9.2, 5.5 Hz, 1H), 1.59 (dt, J = 8.8, 5.6 Hz, 1H). |
| 4.96 | 595.1 | 1H NMR (400 MHz, DMSO-d6) δ 8.80 (d, J = 0.8 Hz, 1H), 8.13-8.08 |
| | | (m, 1H), 8.05 (ddd, J = 8.3, 6.7, 1.8 Hz, 1H), 7.73 (dt, J = 8.4, 1.1 Hz, |
| | | 1H), 7.67 (t, J = 7.8 Hz, 1H), 7.59-7.53 (m, 1H), 7.22 (ddd, J = 8.8, |
| | | 6.5, 1.1 Hz, 1H), 7.04 (ddd, J = 7.7, 6.7, 0.9 Hz, 1H), 6.38 (s, 1H), 4.16 |
| | | (s, 3H), 3.39 (s, 3H), 2.89-2.79 (m, 2H), 2.22 (s, 3H), 2.04 (s, 3H), |
| | | 1.91 (dq, J = 9.6, 5.4 Hz, 2H). |
| 4.97 | 584.1 | 1H NMR (400 MHz, DMSO-d6) δ 8.78 (d, J = 0.8 Hz, 1H), 8.09 (t, J = |
| | | 7.2 Hz, 1H), 8.07-8.00 (m, 1H), 7.75 (s, 1H), 7.66 (t, J = 7.8 Hz, 1H), |
| | | 7.48 (s, 1H), 6.21 (s, 1H), 4.33 (t, J = 6.4 Hz, 2H), 3.38 (s, 3H), 3.04 (t, |
| | | J = 6.4 Hz, 2H), 2.40-2.31 (m, 2H), 2.17 (s, 3H), 2.00 (s, 3H), 1.66- |
| | | 1.47 (m, 2H). |
| 4.98 | 570.0 | 1H NMR (400 MHz, DMSO-d6) δ 8.78 (d, J = 0.8 Hz, 1H), 8.13-8.07 |
| | | (m, 1H), 8.04 (ddd, J = 8.4, 6.8, 1.8 Hz, 1H), 7.70-7.63 (m, 2H), 6.31 |
| | | (s, 1H), 3.98 (s, 3H), 3.38 (s, 3H), 2.62-2.55 (m, 1H), 2.46-2.39 (m, |
| | | 1H), 2.18 (s, 3H), 2.01 (s, 3H), 1.77-1.64 (m, 2H). |
| 4.99 | 543.0 | 1H NMR (400 MHz, DMSO-d6) δ 8.83-8.75 (m, 2H), 8.61-8.55 (m, |
| | | 1H), 8.49 (d, J = 2.6 Hz, 1H), 8.14-8.01 (m, 2H), 7.66 (t, J = 7.8 Hz, |
| | | 1H), 6.35 (s, 1H), 3.38 (s, 3H), 2.84 (t, J = 7.3 Hz, 2H), 2.17 (s, 3H), |
| | | 2.03 (s, 3H), 1.81 (dd, J = 8.4, 6.3 Hz, 2H). |
| 4.64 | 574.8 | 1H NMR (400 MHz, DMSO-d6) δ 8.77 (s, 1H), 8.65 (d, J = 1.8 Hz, |
| | | 1H), 8.16-8.00 (m, 2H), 7.66 (t, J = 7.8 Hz, 1H), 6.34 (s, 1H), 3.38 (s, |
| | | 3H), 2.81 (dddd, J = 15.1, 8.7, 6.0, 4.2 Hz, 2H), 2.46 (d, J = 2.5 Hz, |
| | | 3H), 2.16 (s, 3H), 2.01 (s, 3H), 1.78 (dddd, J = 26.5, 8.7, 6.1, 4.4 Hz, 2H). |
| 4.65 | 566.1 | 1H NMR (400 MHz, DMSO-d6) δ 8.78 (s, 1H), 8.09 (td, J = 7.4, 6.6, |
| | | 1.7 Hz, 1H), 8.04 (ddd, J = 8.4, 6.7, 1.8 Hz, 1H), 7.66 (t, J = 7.8 Hz, |
| | | 1H), 7.36 (d, J = 3.3 Hz, 1H), 6.22 (s, 1H), 3.38 (s, 3H), 2.45-2.38 (m, |
| | | 1H), 2.26-2.19 (m, 1H), 2.18 (s, 3H), 1.99 (s, 3H), 1.60-1.51 (m, 2H). |
| 4.66 | 557.3 | 1H NMR (400 MHz, DMSO) δ 8.79 (s, 1H), 8.56 (s, 1H), 8.39 (s, 1H), |
| | | 8.11 (t, J = 7.2 Hz, 1H), 8.05 (t, J = 7.5 Hz, 1H), 7.67 (t, J = 7.8 Hz, |
| | | 1H), 6.35 (s, 1H), 3.39 (s, 3H), 2.80 (t, J = 7.3 Hz, 2H), 2.49 (s, 3H), |
| | | 2.18 (s, 3H), 2.03 (s, 3H), 1.86-1.73 (m, 2H). |
| 4.67 | 599.2 | 1H NMR (400 MHz, DMSO) δ 8.80 (s, 1H), 8.17 (d, J = 2.4 Hz, 1H), |
| | | 8.11 (t, J = 7.0 Hz, 1H), 8.05 (t, J = 7.1 Hz, 1H), 7.67 (t, J = 7.7 Hz, |
| | | 1H), 7.20-7.10 (m, 1H), 6.93-6.80 (m, 2H), 6.49 (s, 1H), 3.21 (d, J = |
| | | 9.8 Hz, 2H), 2.76-2.64 (m, 1H), 2.51 (t, J = 2.1 Hz, 3H), 2.21 (s, 3H), |
| | | 2.06 (s, 3H), 1.81 (dt, J = 10.5, 5.5 Hz, 1H). |
| 4.68 | 570.1 | 1H NMR (400 MHz, Chloroform-d) δ 8.61 (d, J = 0.8 Hz, 1H), 8.13- |
| | (M + Na)+ | 8.05 (m, 1H), 8.02-7.94 (m, 1H), 7.50 (t, J = 7.8 Hz, 1H), 7.31 (d, J = |
| | | 2.3 Hz, 1H), 6.07 (d, J = 2.3 Hz, 1H), 5.79 (s, 1H), 3.25 (s, 3H), 2.71- |
| | | 2.62 (m, 1H), 2.55-2.45 (m, 1H), 2.24 (s, 3H), 2.02 (s, 3H), 1.70- |
| | | 1.65 (m, 1H), 1.50 (dt, J = 9.1, 5.6 Hz, 1H). |
| 4.69 | 543.1 | 1H NMR (400 MHz, DMSO-d6) δ 9.08 (s, 1H), 8.78 (s, 1H), 8.67 (d, |
| | | J = 5.2 Hz, 1H), 8.14-7.94 (m, 2H), 7.76-7.48 (m, 2H), 6.34 (s, 1H), |
| | | 3.38 (s, 3H), 2.96-2.85 (m, 1H), 2.80-2.70 (m, 1H), 2.16 (s, 3H), |
| | | 2.02 (s, 3H), 1.91-1.77 (m, 2H). |
| 4.70 | 560.1 | 1H NMR (400 MHz, DMSO-d6) δ 8.78 (d, J = 0.8 Hz, 1H), 8.13- |
| | | 8.07 (m, 1H), 8.04 (ddd, J = 8.4, 6.8, 1.8 Hz, 1H), 7.66 (t, J = 7.8 Hz, |
| | | 1H), 6.33-6.23 (m, 1H), 4.01 (s, 3H), 3.38 (s, 3H), 2.63 (ddd, J = 8.7, |
| | | 5.8, 4.5 Hz, 1H), 2.50 (m, 1H), 2.25 (s, 3H), 2.22-2.13 (m, 3H), 2.01 |
| | | (s, 3H), 1.69-1.52 (m, 2H). |
| 4.71 | 561.1 | 1H NMR (400 MHz, DMSO-d6) δ 8.84 (s, 2H), 8.78 (s, 1H), 8.17- |
| | | 7.98 (m, 2H), 7.67 (t, J = 7.8 Hz, 1H), 6.36 (s, 1H), 3.39 (s, 3H), 2.95- |
| | | 2.80 (m, 2H), 2.17 (s, 3H), 2.02 (s, 3H), 1.90-1.69 (m, 2H). |
| 4.72 | 560.1 | 1H NMR (400 MHz, DMSO-d6) δ 8.78 (s, 1H), 8.57 (d, J = 5.1 Hz, |
| | | 1H), 8.15-7.99 (m, 2H), 7.66 (t, J = 7.8 Hz, 1H), 7.24 (d, J = 5.1 Hz, |
| | | 1H), 6.34 (s, 1H), 3.39 (s, 3H), 2.92-2.82 (m, 1H), 2.79-2.70 (m, |
| | | 1H), 2.17 (s, 3H), 2.02 (s, 3H), 1.89-1.79 (m, 1H), 1.79-1.67 (m, 1H). |
| 4.73 | 543.1 | 1H NMR (400 MHz, DMSO-d6) δ 8.99 (s, 1H), 8.72 (s, 3H), 8.10- |
| | | 7.90 (m, 2H), 7.60 (t, J = 7.8 Hz, 1H), 6.27 (s, 1H), 3.32 (s, 3H), 2.64- |
| | | 2.53 (m, 1H), 2.52-2.45 (m, 1H), 2.11 (s, 3H), 1.96 (s, 3H), 1.85- |
| | | 1.78 (m, 1H), 1.78-1.61 (m, 1H). |
| 4.74 | 569.7 | 1H NMR (400 MHz, Methanol-d4) δ 8.72 (d, J = 0.8 Hz, 1H), 8.16- |
| | | 8.04 (m, 2H), 7.63 (t, J = 7.8 Hz, 1H), 6.87 (s, 1H), 6.24 (s, 1H), 4.02 |
| | | (s, 3H), 3.31 (s, 3H), 2.80-2.71 (m, 1H), 2.58-2.50 (m, 1H), 2.24 (s, |
| | | 3H), 2.10 (s, 3H), 1.77 (dt, J = 8.5, 5.5 Hz, 1H), 1.70 (dt, J = 9.1, 5.5 |
| | | Hz, 1H). |
| 4.75 | 558.7 | 1H NMR (400 MHz, Methanol-d4) δ 8.72 (d, J = 0.8 Hz, 1H), 8.16- |
| | | 8.04 (m, 2H), 7.63 (t, J = 7.8 Hz, 1H), 6.22 (s, 1H), 6.00 (s, 1H), 3.76 |
| | | (s, 3H), 2.69-2.63 (m, 1H), 2.43 (dd, J = 13.5, 6.5 Hz, 1H), 2.30 (s, |
| | | 3H), 2.24 (d, J = 0.7 Hz, 3H), 2.10 (s, 3H), 1.73-1.63 (m, 2H), 1.31 (s, 3H). |
| 4.76 | 558.7 | 1H NMR (400 MHz, Methanol-d4) δ 8.71 (d, J = 0.8 Hz, 1H), 8.09 |
| | | (dddd, J = 22.2, 8.3, 6.6, 1.8 Hz, 2H), 7.62 (t, J = 7.8 Hz, 1H), 7.39 (s, |
| | | 1H), 6.22 (s, 1H), 3.81 (s, 3H), 3.30 (s, 3H), 2.50 (dt, J = 8.6, 5.1 Hz, |
| | | 1H), 2.35 (s, 3H), 2.25 (s, 4H), 2.09 (s, 3H), 1.65 (dt, J = 9.0, 5.3 Hz, |
| | | 1H), 1.59-1.52 (m, 1H). |
| 4.77 | 543.0 | 1H NMR (400 MHz, DMSO-d6) δ 9.28 (dd, J = 2.6, 1.2 Hz, 1H), 9.12 |
| | | (dd, J = 5.5, 1.1 Hz, 1H), 8.79 (s, 1H), 8.14-8.01 (m, 2H), 7.72-7.58 |
| | | (m, 2H), 6.35 (s, 1H), 3.38 (s, 3H), 2.72 (td, J = 7.7, 4.7 Hz, 1H), 2.58 |
| | | (td, J = 7.7, 4.6 Hz, 1H), 2.17 (d, J = 2.4 Hz, 3H), 2.02 (q, J = 4.6, 3.8 |
| | | Hz, 3H), 1.92-1.87 (m, 2H). |
| 4.78 | 556.0 | 1H NMR (400 MHz, DMSO-d6) δ 8.78 (s, 1H), 8.15-8.01 (m, 2H), |
| | | 7.81 (s, 1H), 7.66 (t, J = 7.8 Hz, 1H), 7.37 (d, J = 7.8 Hz, 1H), 7.26 (s, |
| | | 1H), 6.32 (s, 1H), 3.38 (s, 3H), 2.81 (ddd, J = 8.9, 6.1, 4.2 Hz, 1H), 2.72 |
| | | (dt, J = 9.3, 5.2 Hz, 1H), 2.16 (d, J = 2.4 Hz, 3H), 2.02 (s, 3H), 1.95- |
| | | 1.70 (m, 2H). |
| 4.79 | 561.1 | 1H NMR (400 MHz, DMSO-d6) δ 8.99 (d, J = 2.9 Hz, 1H), 8.88-8.66 |
| | | (m, 2H), 8.20-8.02 (m, 2H), 7.67 (t, J = 7.8 Hz, 1H), 6.44 (s, 1H), |
| | | 3.39 (s, 3H), 3.09-2.98 (m, 1H), 2.98-2.86 (m, 1H), 2.18 (s, 3H), |
| | | 2.03 (s, 3H), 1.95-1.82 (m, 2H). |
| 4.80 | 543.0 | 1H NMR (400 MHz, DMSO-d6) δ 9.10 (dd, J = 4.9, 1.6 Hz, 1H), 8.78 |
| | | (d, J = 0.8 Hz, 1H), 8.13-8.07 (m, 1H), 8.07-8.02 (m, 1H), 7.75 (dd, |
| | | J = 8.5, 1.7 Hz, 1H), 7.70-7.60 (m, 2H), 6.37 (s, 1H), 3.39 (s, 3H), |
| | | 3.00-2.92 (m, 1H), 2.89-2.80 (m, 1H), 2.17 (s, 3H), 2.03 (s, 3H), |
| | | 2.00-1.94 (m, 1H), 1.88-1.79 (m, 1H). |
| 4.81 | 571.1 | 1H NMR (400 MHz, DMSO-d6) δ 8.78 (s, 1H), 8.09 (t, J = 7.3 Hz, |
| | | 1H), 8.07-8.01 (m, 1H), 7.71 (s, 1H), 7.66 (t, J = 7.8 Hz, 1H), 7.37 (s, |
| | | 1H), 6.19 (s, 1H), 3.69-3.61 (m, 1H), 3.38 (s, 3H), 2.38-2.27 (m, |
| | | 2H), 2.17 (s, 3H), 1.99 (s, 3H), 1.62-1.52 (m, 2H), 1.03-0.97 (m, |
| | | 2H), 0.97-0.88 (m, 2H). |
| 4.82 | 563.0 | 1H NMR (400 MHz, DMSO-d6) δ 8.78 (d, J = 0.8 Hz, 1H), 8.12- |
| | | 8.07 (m, 1H), 8.07-8.01 (m, 1H), 7.66 (t, J = 7.8 Hz, 1H), 7.36 (d, J = |
| | | 3.3 Hz, 1H), 6.22 (s, 1H), 3.67 (s, 3H), 3.38 (s, 3H), 2.41 (ddd, J = 8.4, |
| | | 5.9, 4.4 Hz, 1H), 2.26-2.19 (m, 1H), 2.18 (s, 3H), 1.99 (s, 3H), 1.62- |
| | | 1.49 (m, 2H). |
| 4.83 | 572.1 | 1H NMR (400 MHz, DMSO-d6) δ 8.79 (d, J = 0.9 Hz, 1H), 8.10 (t, J = |
| | | 7.6 Hz, 1H), 8.05 (t, J = 7.3 Hz, 1H), 7.66 (t, J = 7.8 Hz, 1H), 7.37 (dd, |
| | | J = 9.1, 6.9 Hz, 1H), 6.36 (m, 2H), 6.22 (d, J = 6.9 Hz, 1H), 3.51 (s, |
| | | 3H), 3.38 (s, 3H), 2.70-2.61 (m, 1H), 2.61-2.53 (m, 1H), 2.18 (s, |
| | | 3H), 2.02 (s, 3H), 1.73 (m, 2H). |
| 4.84 | 582.0 | 1H NMR (400 MHz, DMSO-d6) δ 8.78 (d, J = 0.8 Hz, 1H), 8.66 (s, |
| | | 1H), 8.45-8.00 (m, 3H), 7.66 (t, J = 7.8 Hz, 1H), 6.31 (s, 1H), 3.38 (s, |
| | | 3H), 2.75-2.62 (m, 2H), 2.17 (s, 3H), 2.01 (s, 3H), 1.89-1.65 (m, 2H). |
| 4.85 | 582.0 | 1H NMR (400 MHz, DMSO-d6) δ 8.78 (d, J = 0.8 Hz, 1H), 8.24- |
| | | 7.92 (m, 4H), 7.66 (t, J = 7.8 Hz, 1H), 6.32 (s, 1H), 3.38 (s, 3H), 2.81- |
| | | 2.62 (m, 2H), 2.21-2.14 (m, 3H), 2.01 (s, 3H), 1.78 (t, J = 7.6 Hz, 2H). |
| 4.86 | 586.1 | 1H NMR (400 MHz, DMSO-d6) δ 8.78 (d, J = 0.8 Hz, 1H), 8.13- |
| | | 8.07 (m, 1H), 8.07-8.01 (m, 1H), 7.70 (s, 1H), 7.66 (t, J = 7.8 Hz, |
| | | 1H), 6.27 (s, 1H), 4.22 (d, J = 7.1 Hz, 2H), 3.38 (s, 3H), 2.66-2.56 (m, |
| | | 2H), 2.22-2.11 (m, 3H), 2.01 (s, 3H), 1.78-1.63 (m, 2H), 1.29 (qq, J = |
| | | 7.4, 3.6, 2.8 Hz, 1H), 0.60-0.48 (m, 2H), 0.37 (dt, J = 6.1, 4.6 Hz, 2H). |
| 4.87 | 574.1 | 1H NMR (400 MHz, DMSO-d6) δ 8.78 (d, J = 0.8 Hz, 1H), 8.13- |
| | | 8.07 (m, 1H), 8.07-8.01 (m, 1H), 7.71-7.62 (m, 2H), 6.30-6.22 (m, |
| | | 1H), 4.75 (hept, J = 6.7 Hz, 1H), 3.38 (s, 3H), 2.69-2.54 (m, 2H), 2.21- |
| | | 2.10 (m, 3H), 2.00 (s, 3H), 1.75-1.61 (m, 2H), 1.46 (dd, J = 6.6, 1.3 |
| | | Hz, 6H). |
| 4.88 | 574.1 | 1H NMR (400 MHz, DMSO-d6) δ 8.78 (d, J = 0.8 Hz, 1H), 8.10 (ddd, |
| | | J = 8.0, 6.7, 1.8 Hz, 1H), 8.04 (ddd, J = 8.2, 6.7, 1.8 Hz, 1H), 7.70 (s, |
| | | 1H), 7.66 (t, J = 7.8 Hz, 1H), 6.27 (s, 1H), 4.31 (t, J = 6.9 Hz, 2H), 3.38 |
| | | (s, 3H), 2.59 (dtd, J = 11.8, 7.6, 3.8 Hz, 2H), 2.21-2.13 (m, 3H), 2.00 |
| | | (s, 3H), 1.85 (h, J = 7.2 Hz, 2H), 1.69 (t, J = 7.5 Hz, 2H), 0.83 (t, J = |
| | | 7.4 Hz, 3H). |
| 4.89 | 560.0 | 1H NMR (400 MHz, DMSO-d6) δ 8.78 (d, J = 0.8 Hz, 1H), 8.10 (ddd, |
| | | J = 7.9, 6.7, 1.8 Hz, 1H), 8.04 (ddd, J = 8.3, 6.8, 1.8 Hz, 1H), 7.70 (s, |
| | | 1H), 7.66 (t, J = 7.8 Hz, 1H), 6.27 (d, J = 1.0 Hz, 1H), 4.38 (q, J = 7.3 |
| | | Hz, 2H), 3.38 (s, 3H), 2.67-2.54 (m, 2H), 2.21-2.12 (m, 3H), 2.01 (s, |
| | | 3H), 1.69 (ddd, J = 8.8, 6.5, 2.3 Hz, 2H), 1.43 (t, J = 7.3 Hz, 3H). |
| 4.90 | 562.0 | 1H NMR (400 MHz, DMSO-d6) δ 8.81-8.75 (m, 1H), 8.16-8.03 (m, |
| | | 2H), 7.67 (t, J = 7.8 Hz, 1H), 7.30 (s, 1H), 6.31-6.21 (m, 1H), 3.39 (s, |
| | | 3H), 2.78-2.66 (m, 2H), 2.64 (s, 3H), 2.24-2.14 (m, 3H), 2.01 (s, |
| | | 3H), 1.86-1.71 (m, 1H), 1.71-1.52 (m, 1H). |
| 4.91 | 548.0 | 1H NMR (400 MHz, DMSO-d6) δ 9.04 (d, J = 4.6 Hz, 1H), 8.81-8.77 |
| | | (m, 1H), 8.17-8.02 (m, 2H), 7.67 (t, J = 7.8 Hz, 1H), 7.41 (d, J = 4.7 |
| | | Hz, 1H), 6.37-6.17 (m, 1H), 3.39 (s, 3H), 2.98-2.84 (m, 1H), 2.84- |
| | | 2.69 (m, 1H), 2.22-2.13 (m, 3H), 2.02 (s, 3H), 1.89-1.80 (m, 1H), |
| | | 1.80-1.58 (m, 1H). |
| 4.92 | 571.0 | 1H NMR (400 MHz, DMSO-d6) δ 8.79 (d, J = 0.8 Hz, 1H), 8.14-8.07 |
| | | (m, 1H), 8.05 (td, J = 7.3, 6.6, 1.8 Hz, 1H), 7.67 (t, J = 7.8 Hz, 1H), |
| | | 7.29 (d, J = 1.8 Hz, 1H), 6.33 (s, 1H), 6.09 (d, J = 1.8 Hz, 1H), 3.64- |
| | | 3.54 (m, 1H), 3.39 (s, 3H), 2.67-2.60 (m, 1H), 2.48-2.40 (m, 1H), |
| | | 2.17 (s, 3H), 2.02 (s, 3H), 1.82-1.74 (m, 1H), 1.68 (dt, J = 8.8, 5.7 Hz, |
| | | 1H), 1.11-1.02 (m, 1H), 1.02-0.85 (m, 3H). |
| 4.93 | 563.1 | 1H NMR (400 MHz, DMSO-d6) δ 8.79 (d, J = 0.8 Hz, 1H), 8.13-8.07 |
| | | (m, 1H), 8.04 (td, J = 7.3, 6.7, 1.8 Hz, 1H), 7.66 (t, J = 7.8 Hz, 1H), |
| | | 6.32 (s, 1H), 5.85 (d, J = 5.7 Hz, 1H), 3.72 (d, J = 1.0 Hz, 3H), 3.38 (s, |
| | | 3H), 2.60-2.52 (m, 2H), 2.18 (s, 3H), 2.01 (s, 3H), 1.71 (dt, J = 9.0, |
| | | 5.4 Hz, 1H), 1.59 (dt, J = 8.9, 5.6 Hz, 1H). |
| 4.94 | 569.9 | 1H NMR (400 MHz, DMSO-d6) δ 8.78 (d, J = 0.8 Hz, 1H), 8.09 (td, J = |
| | | 7.7, 7.2, 1.8 Hz, 1H), 8.04 (td, J = 7.3, 6.6, 1.8 Hz, 1H), 7.79 (s, 1H), |
| | | 7.66 (t, J = 7.8 Hz, 1H), 7.57 (s, 1H), 6.21 (s, 1H), 5.43 (s, 2H), 3.38 (s, |
| | | 3H), 2.37 (t, J = 7.4 Hz, 2H), 2.17 (s, 3H), 2.00 (s, 3H), 1.65-1.51 (m, 2H). |
| 4.95 | 570.0 | 1H NMR (400 MHz, DMSO-d6) δ 8.78 (d, J = 0.8 Hz, 1H), 8.10 (td, J = |
| | | 7.3, 6.6, 1.8 Hz, 1H), 8.04 (ddd, J = 8.3, 6.7, 1.8 Hz, 1H), 7.96 (s, |
| | | 1H), 7.66 (t, J = 7.8 Hz, 1H), 6.30 (s, 1H), 3.91 (s, 3H), 3.38 (s, 3H), |
| | | 2.56-2.51 (m, 1H), 2.39 (dt, J = 10.5, 5.4 Hz, 1H), 2.17 (s, 3H), 2.01 |
| | | (s, 3H), 1.70 (dt, J = 9.2, 5.5 Hz, 1H), 1.59 (dt, J = 8.8, 5.6 Hz, 1H). |
| 4.96 | 595.1 | 1H NMR (400 MHz, DMSO-d6) δ 8.80 (d, J = 0.8 Hz, 1H), 8.13-8.08 |
| | | (m, 1H), 8.05 (ddd, J = 8.3, 6.7, 1.8 Hz, 1H), 7.73 (dt, J = 8.4, 1.1 Hz, |
| | | 1H), 7.67 (t, J = 7.8 Hz, 1H), 7.59-7.53 (m, 1H), 7.22 (ddd, J = 8.8, |
| | | 6.5, 1.1 Hz, 1H), 7.04 (ddd, J = 7.7, 6.7, 0.9 Hz, 1H), 6.38 (s, 1H), 4.16 |
| | | (s, 3H), 3.39 (s, 3H), 2.89-2.79 (m, 2H), 2.22 (s, 3H), 2.04 (s, 3H), |
| | | 1.91 (dq, J = 9.6, 5.4 Hz, 2H). |
| 4.97 | 584.1 | 1H NMR (400 MHz, DMSO-d6) δ 8.78 (d, J = 0.8 Hz, 1H), 8.09 (t, J = |
| | | 7.2 Hz, 1H), 8.07-8.00 (m, 1H), 7.75 (s, 1H), 7.66 (t, J = 7.8 Hz, 1H), |
| | | 7.48 (s, 1H), 6.21 (s, 1H), 4.33 (t, J = 6.4 Hz, 2H), 3.38 (s, 3H), 3.04 (t, |
| | | J = 6.4 Hz, 2H), 2.40-2.31 (m, 2H), 2.17 (s, 3H), 2.00 (s, 3H), 1.66- |
| | | 1.47 (m, 2H). |
| 4.98 | 570.0 | 1H NMR (400 MHz, DMSO-d6) δ 8.78 (d, J = 0.8 Hz, 1H), 8.13-8.07 |
| | | (m, 1H), 8.04 (ddd, J = 8.4, 6.8, 1.8 Hz, 1H), 7.70-7.63 (m, 2H), 6.31 |
| | | (s, 1H), 3.98 (s, 3H), 3.38 (s, 3H), 2.62-2.55 (m, 1H), 2.46-2.39 (m, |
| | | 1H), 2.18 (s, 3H), 2.01 (s, 3H), 1.77-1.64 (m, 2H). |
| 4.99 | 543.0 | 1H NMR (400 MHz, DMSO-d6) δ 8.83-8.75 (m, 2H), 8.61-8.55 (m, |
| | | 1H), 8.49 (d, J = 2.6 Hz, 1H), 8.14-8.01 (m, 2H), 7.66 (t, J = 7.8 Hz, |
| | | 1H), 6.35 (s, 1H), 3.38 (s, 3H), 2.84 (t, J = 7.3 Hz, 2H), 2.17 (s, 3H), |
| | | 2.03 (s, 3H), 1.81 (dd, J = 8.4, 6.3 Hz, 2H). |
| 4.100 | 610.1 | 1H NMR (400 MHz, DMSO-d6) δ 11.32 (s, 1H), 8.79 (d, J = 0.8 Hz, |
| | | 1H), 8.14-8.08 (m, 1H), 8.05 (ddd, J = 8.3, 6.8, 1.8 Hz, 1H), 7.77 (t, J = |
| | | 7.6 Hz, 1H), 7.70-7.61 (m, 2H), 7.57 (d, J = 7.9 Hz, 1H), 6.36 (d, J = |
| | | 0.9 Hz, 1H), 3.51 (dt, J = 9.0, 6.2 Hz, 1H), 3.39 (s, 3H), 2.78-2.63 |
| | | (m, 1H), 2.22-2.14 (m, 3H), 2.05 (s, 3H), 1.90 (dt, J = 15.4, 6.4 Hz, 2H). |
| 4.101 | 593.0 | 1H NMR (400 MHz, DMSO-d6) δ 8.78 (d, J = 0.8 Hz, 1H), 8.10 (t, J = |
| | | 7.3 Hz, 1H), 8.07-7.99 (m, 1H), 7.78-7.45 (m, 2H), 6.35 (s, 1H), |
| | | 3.38 (s, 3H), 2.97 (s, 1H), 2.82-2.71 (m, 1H), 2.21-2.14 (m, 3H), |
| | | 2.02 (s, 3H), 1.78 (t, J = 7.5 Hz, 2H). |
| 4.102 | 588.0 | 1H NMR (400 MHz, DMSO-d6) δ 8.71 (s, 1H), 8.07-7.94 (m, 2H), |
| | | 7.79 (s, 1H), 7.59 (t, J = 7.8 Hz, 1H), 7.41 (s, 1H), 6.67 (s, 1H), 6.18 (s, |
| | | 1H), 3.92 (s, 3H), 3.32 (s, 3H), 2.51-2.45 (m, 2H), 2.10 (s, 3H), 1.93 |
| | | (s, 3H), 1.62-1.46 (m, 2H). |
| 4.103 | 543.0 | 1H NMR (400 MHz, DMSO-d6) δ 8.77 (s, 1H), 8.74 (d, J = 4.9 Hz, |
| | | 2H), 8.13-8.02 (m, 2H), 7.66 (t, J = 7.8 Hz, 1H), 7.37 (t, J = 4.9 Hz, |
| | | 1H), 6.35 (s, 1H), 2.91 (ddd, J = 8.9, 6.2, 4.2 Hz, 1H), 2.79 (ddd, J = |
| | | 9.4, 5.8, 4.2 Hz, 1H), 2.16 (s, 3H), 2.02 (s, 3H), 1.89-1.76 (m, 2H). |
| 4.104 | 557.0 | 1H NMR (400 MHz, DMSO-d6) δ 8.77 (s, 1H), 8.57 (d, J = 5.1 Hz, |
| | | 1H), 8.13-8.01 (m, 2H), 7.66 (t, J = 7.8 Hz, 1H), 7.23 (d, J = 5.1 Hz, |
| | | 1H), 6.33 (s, 1H), 2.92-2.82 (m, 1H), 2.79-2.70 (m, 1H), 2.44 (s, |
| | | 3H), 2.16 (s, 3H), 2.01 (s, 3H), 1.84 (dt, J = 9.6, 4.5 Hz, 1H), 1.78- |
| | | 1.70 (m, 1H). |
| 4.105 | 572.0 | 1H NMR (400 MHz, DMSO-d6) δ 11.63 (s, 1H), 8.78 (d, J = 0.8 Hz, |
| | | 1H), 8.13-8.07 (m, 1H), 8.04 (ddd, J = 8.4, 6.7, 1.8 Hz, 1H), 7.66 (t, J = |
| | | 7.8 Hz, 1H), 7.14 (d, J = 7.0 Hz, 1H), 6.24 (s, 1H), 5.96 (d, J = 7.0 |
| | | Hz, 1H), 3.38 (s, 3H), 2.55-2.40 (m, 2H), 2.19 (d, J = 0.7 Hz, 3H), |
| | | 2.17-2.13 (m, 3H), 2.01 (s, 3H), 1.75 (dt, J = 7.8, 5.5 Hz, 1H), 1.53 |
| | | (dt, J = 10.1, 5.1 Hz, 1H). |
| 4.106 | 549.0 | 1H NMR (400 MHz, DMSO-d6) δ 8.78 (d, J = 0.9 Hz, 1H), 8.18-7.98 |
| | | (m, 2H), 7.73-7.60 (m, 2H), 6.27 (s, 1H), 3.38 (s, 3H), 2.65-2.55 (m, |
| | | 2H), 2.17 (s, 3H), 2.00 (s, 3H), 1.69 (t, J = 7.5 Hz, 2H). |
| 4.107 | 557.3 | 1H NMR (400 MHz, DMSO-d6) δ 13.73 (s, 1H), 8.80 (s, 1H), 8.15- |
| | | 8.08 (m, 1H), 8.08-8.02 (m, 1H), 7.76 (s, 2H), 7.67 (t, J = 7.8 Hz, |
| | | 1H), 6.89-6.56 (m, 2H), 6.34 (s, 1H), 3.39 (s, 3H), 2.79-2.70 (m, |
| | | 1H), 2.68-2.56 (m, 1H), 2.17 (s, 3H), 2.03 (s, 3H), 1.98-1.89 (m, |
| | | 1H), 1.88-1.70 (m, 1H). |
| 4.108 | 586.7 | 1H NMR (400 MHz, DMSO-d6) δ 9.43 (s, 1H), 8.81 (s, 1H), 8.16- |
| | | 8.01 (m, 2H), 7.73 (s, 1H), 7.68 (t, J = 7.8 Hz, 1H), 7.48 (s, 1H), 6.37 |
| | | (s, 1H), 3.39 (s, 3H), 2.82-2.73 (m, 1H), 2.73-2.64 (m, 1H), 2.22 (s, |
| | | 3H), 2.05 (s, 3H), 1.89-1.75 (m, 2H). |
| 4.109 | 531.0 | 1H NMR (400 MHz, Acetonitrile-d3) δ 8.58 (d, J = 0.8 Hz, 1H), 8.00- |
| | | 7.88 (m, 2H), 7.49 (t, J = 7.8 Hz, 1H), 7.40 (s, 2H), 5.94-5.78 (m, |
| | | 1H), 3.15 (s, 3H), 2.47-2.29 (m, 1H), 2.28-2.16 (m, 1H), 2.10 (s, |
| | | 3H), 1.90 (s, 3H), 1.52-1.33 (m, 2H). |
| 4.110 | 578.0 | 1H NMR (400 MHz, Chloroform-d) δ 8.64 (s, 1H), 8.27 (d, J = 2.4 Hz, |
| | | 1H), 8.10 (ddd, J = 8.2, 6.6, 1.8 Hz, 1H), 7.98 (ddd, J = 8.1, 6.8, 1.8 Hz, |
| | | 1H), 7.51 (t, J = 7.8 Hz, 1H), 7.20 (ddd, J = 9.3, 8.0, 2.4 Hz, 1H), 5.89 |
| | | (s, 1H), 3.25 (s, 3H), 3.01 (ddd, J = 8.9, 6.1, 4.4 Hz, 1H), 2.77-2.69 |
| | | (m, 1H), 2.26 (s, 3H), 2.05 (s, 3H), 1.99 (ddd, J = 8.9, 5.9, 4.8 Hz, 1H), |
| | | 1.58 (ddd, J = 8.8, 6.0, 4.9 Hz, 1H). |
| 4.111 | 548.2 | 1H NMR (400 MHz, DMSO-d6) δ 8.78 (d, J = 0.8 Hz, 1H), 8.18-8.00 |
| | | (m, 2H), 7.67 (t, J = 7.8 Hz, 1H), 7.62 (d, J = 0.8 Hz, 1H), 7.37 (d, J = |
| | | 0.8 Hz, 1H), 6.20 (s, 1H), 3.39 (s, 3H), 2.37-2.29 (m, 2H), 2.18 (s, |
| | | 3H), 2.00 (s, 3H), 1.65-1.46 (m, 2H). |
| 4.112 | 559.0 | 1H NMR (400 MHz, DMSO-d6) δ 12.29 (s, 1H), 8.78 (d, J = 0.8 Hz, |
| | | 1H), 8.13-8.07 (m, 1H), 8.07-8.01 (m, 1H), 7.66 (t, J = 7.8 Hz, 1H), |
| | | 7.28 (m, 2H), 6.38-6.26 (m, 1H), 3.38 (s, 3H), 3.06 (dt, J = 9.6, 5.6 |
| | | Hz, 1H), 2.79-2.72 (m, 1H), 2.21-2.13 (m, 3H), 2.02 (s, 3H), 1.82- |
| | | 1.64 (m, 2H). |
| 4.113 | 576.0 | 1H NMR (400 MHz, DMSO-d6) δ 8.78 (d, J = 0.8 Hz, 1H), 8.14- |
| | | 8.07 (m, 1H), 8.04 (ddd, J = 8.3, 6.7, 1.8 Hz, 1H), 7.66 (t, J = 7.8 Hz, |
| | | 1H), 7.54 (t, J = 9.0 Hz, 1H), 6.48 (dd, J = 8.8, 3.0 Hz, 1H), 6.35 (d, J = |
| | | 1.0 Hz, 1H), 3.38 (s, 3H), 2.85 (ddd, J = 8.8, 6.0, 4.2 Hz, 1H), 2.80- |
| | | 2.70 (m, 1H), 2.22-2.12 (m, 3H), 2.02 (s, 3H), 1.79 (dt, J = 9.4, 4.6 |
| | | Hz, 1H), 1.70 (dt, J = 9.3, 4.8 Hz, 1H). |
| 4.114 | 576.0 | 1H NMR (400 MHz, DMSO-d6) δ 11.79 (s, 1H), 8.78 (d, J = 0.8 Hz, |
| | | 1H), 8.14-8.07 (m, 1H), 8.04 (ddd, J = 8.3, 6.7, 1.8 Hz, 1H), 7.66 (t, J = |
| | | 7.8 Hz, 1H), 7.40 (t, J = 7.7 Hz, 1H), 6.30-6.19 (m, 2H), 3.38 (s, |
| | | 3H), 3.13 (dt, J = 8.6, 5.4 Hz, 1H), 2.41-2.29 (m, 1H), 2.18 (s, 3H), |
| | | 2.08 (ddd, J = 8.5, 6.6, 4.4 Hz, 1H), 2.00 (s, 3H), 1.51 (dt, J = 9.7, 5.0 |
| | | Hz, 1H). |
| 4.115 | 576.0 | 1H NMR (400 MHz, DMSO-d6) δ 11.21 (s, 1H), 8.78 (d, J = 0.8 Hz, |
| | | 1H), 8.14-8.07 (m, 1H), 8.07-8.00 (m, 1H), 7.71-7.57 (m, 2H), |
| | | 6.55 (dd, J = 8.2, 1.2 Hz, 1H), 6.29 (s, 1H), 3.38 (s, 3H), 2.56-2.42 |
| | | (m, 2H), 2.18 (s, 3H), 2.01 (s, 3H), 1.76-1.60 (m, 2H). |
| 4.116 | 572.1 | 1H NMR (400 MHz, DMSO-d6) δ 8.78 (s, 1H), 8.13-8.07 (m, 1H), |
| | | 8.04 (ddd, J = 8.3, 6.8, 1.8 Hz, 1H), 7.66 (t, J = 7.8 Hz, 1H), 7.13 (d, J = |
| | | 2.4 Hz, 1H), 7.03 (d, J = 2.4 Hz, 1H), 6.26 (s, 1H), 3.38 (s, 3H), 2.61- |
| | | 2.54 (m, 1H), 2.50-2.43 (m, 1H), 2.18 (s, 3H), 2.01 (s, 6H), 1.80 (ddd, |
| | | J = 8.6, 6.7, 4.8 Hz, 1H), 1.56 (dt, J = 10.1, 5.1 Hz, 1H). |
| 4.117 | 581.0 | 1H NMR (400 MHz, DMSO-d6) δ 8.79 (s, 1H), 8.70 (s, 1H), 8.14- |
| | | 8.01 (m, 2H), 7.95 (d, J = 2.2 Hz, 1H), 7.70-7.63 (m, 2H), 7.15 (dd, J = |
| | | 9.2, 1.6 Hz, 1H), 6.58 (d, J = 2.3 Hz, 1H), 6.32 (s, 1H), 3.39 (s, 3H), |
| | | 2.64-2.53 (m, 2H), 2.18 (s, 3H), 2.03 (s, 3H), 1.84 (dt, J = 8.6, 5.8 Hz, |
| | | 1H), 1.77-1.69 (m, 1H). |
| 4.118 | 599.8 | 1H NMR (400 MHz, DMSO-d6) δ 9.19 (dd, J = 3.8, 2.3 Hz, 1H), 8.79 |
| | | (s, 1H), 8.56 (s, 1H), 8.15-8.08 (m, 1H), 8.08-8.01 (m, 1H), 7.77 |
| | | (dd, J = 9.6, 2.4 Hz, 1H), 7.67 (t, J = 7.8 Hz, 1H), 6.41 (s, 1H), 3.39 (s, |
| | | 3H), 3.17-3.08 (m, 1H), 3.04-2.95 (m, 1H), 2.30-2.21 (m, 1H), |
| | | 2.19 (s, 3H), 2.05 (s, 3H), 1.93-1.83 (m, 1H). |
| 4.119 | 615.7 | 1H NMR (400 MHz, DMSO-d6) δ 8.79 (d, J = 0.5 Hz, 1H), 8.34 (d, J = |
| | | 1.3 Hz, 1H), 8.15-8.08 (m, 1H), 8.08-8.00 (m, 1H), 7.83 (d, J = 1.2 |
| | | Hz, 1H), 7.67 (t, J = 7.8 Hz, 1H), 7.37 (s, 1H), 6.42 (s, 1H), 3.39 (s, |
| | | 3H), 3.34-3.28 (m, 1H), 3.07-2.97 (m, 1H), 2.40-2.35 (m, 1H), |
| | | 2.18 (s, 3H), 2.05 (s, 3H), 2.01-1.91 (m, 1H) |
| 4.120 | 598.8 | 1H NMR (400 MHz, DMSO-d6) δ 8.89 (dd, J = 4.6, 2.0 Hz, 1H), 8.80 |
| | | (s, 1H), 8.15-8.08 (m, 1H), 8.08-8.01 (m, 2H), 7.67 (t, J = 7.8 Hz, |
| | | 1H), 7.23 (dd, J = 9.5, 2.1 Hz, 1H), 6.86-6.83 (m, 1H), 6.43 (s, 1H), |
| | | 3.39 (s, 3H), 2.88-2.79 (m, 1H), 2.74-2.66 (m, 1H), 2.21 (s, 3H), |
| | | 2.05 (s, 3H), 1.98-1.89 (m, 1H), 1.85-1.77 (m, 1H). |
| 4.121 | 605.8 | 1H NMR (400 MHz, DMSO-d6) δ 8.86 (d, J = 6.9 Hz, 1H), 8.77 (s, |
| | | 1H), 8.66 (s, 1H), 8.14-8.08 (m, 1H), 8.08-8.01 (m, 1H), 7.67 (t, J = |
| | | 7.8 Hz, 1H), 7.51 (d, J = 7.2 Hz, 1H), 7.21 (t, J = 7.0 Hz, 1H), 6.29 (s, |
| | | 1H), 3.45-3.35 (3H), 2.85-2.75 (m, 1H), 2.47-2.38 (m, 1H), 2.17 |
| | | (s, 3H), 2.15-2.06 (m, 1H), 2.01 (s, 3H), 1.94-1.82 (m, 1H). |
| 4.122 | 581.8 | 1H NMR (400 MHz, DMSO-d6) δ 9.32 (s, 1H), 8.79 (s, 1H), 8.48 (d, J = |
| | | 6.8 Hz, 1H), 8.16-8.08 (m, 1H), 8.08-8.02 (m, 1H), 7.67 (t, J = 7.8 |
| | | Hz, 1H), 7.31 (d, J = 6.9 Hz, 1H), 6.98 (t, J = 6.9 Hz, 1H), 6.41 (s, 1H), |
| | | 3.40 (s, 3H), 3.13-3.05 (m, 1H), 3.00-2.93 (m, 1H), 2.29-2.21 (m, |
| | | 1H), 2.20 (s, 3H), 2.06 (s, 3H), 1.86-1.78 (m, 1H). |
| 4.123 | 581.0 | 1H NMR (400 MHz, DMSO-d6) δ 8.80 (s, 2H), 8.43 (s, 1H), 8.15- |
| | | 8.02 (m, 2H), 7.73-7.64 (m, 3H), 6.94 (d, J = 9.9 Hz, 1H), 6.33 (s, |
| | | 1H), 3.39 (s, 3H), 2.60-2.53 (m, 2H), 2.19 (s, 3H), 2.03 (s, 3H), 1.81- |
| | | 1.70 (m, 2H). |
| 4.124 | 581.0 | 1H NMR (400 MHz, DMSO-d6) δ 8.78 (s, 1H), 8.61 (d, J = 7.3 Hz, |
| | | 1H), 8.14-8.01 (m, 2H), 7.96 (d, J = 2.2 Hz, 1H), 7.66 (t, J = 7.8 Hz, |
| | | 1H), 7.60 (d, J = 1.9 Hz, 1H), 6.80 (dd, J = 7.3, 2.0 Hz, 1H), 6.50 (d, J = |
| | | 2.2 Hz, 1H), 6.32 (s, 1H), 3.38 (s, 3H), 2.60 (t, J = 7.4 Hz, 2H), 2.18 (s, |
| | | 3H), 2.03 (s, 3H), 1.87-1.76 (m, 2H). |
| 4.125 | 581.0 | 1H NMR (400 MHz, DMSO-d6) δ 8.79 (s, 1H), 8.62 (d, J = 2.2 Hz, |
| | | 1H), 8.13-8.09 (m, 2H), 8.09-8.02 (m, 1H), 7.66 (t, J = 7.8 Hz, 1H), |
| | | 7.40 (dd, J = 2.9, 1.5 Hz, 1H), 6.92 (dd, J = 4.0, 2.9 Hz, 1H), 6.50- |
| | | 6.44 (m, 1H), 6.32 (s, 1H), 3.39 (s, 3H), 2.56-2.52 (m, 2H), 2.19 (s, |
| | | 3H), 2.02 (s, 3H), 1.82-1.66 (m, 2H). |
| 4.126 | 571.1 | 1H NMR (400 MHz, DMSO-d6) δ 8.78 (d, J = 0.8 Hz, 1H), 8.12-8.07 |
| | | (m, 1H), 8.04 (ddd, J = 8.3, 6.7, 1.8 Hz, 1H), 7.66 (t, J = 7.8 Hz, 1H), |
| | | 7.36 (s, 1H), 6.19 (s, 1H), 4.00 (t, J = 7.3 Hz, 2H), 3.38 (s, 3H), 2.84 |
| | | (dd, J = 8.8, 6.3 Hz, 2H), 2.58-2.51 (m, 2H), 2.38 (dt, J = 8.6, 5.1 Hz, |
| | | 1H), 2.29-2.22 (m, 1H), 2.17 (s, 3H), 1.99 (s, 3H), 1.56 (dt, J = 9.2, |
| | | 5.1 Hz, 1H), 1.50 (dt, J = 8.4, 5.6 Hz, 1H). |
| 4.127 | 576.1 | 1H NMR (400 MHz, DMSO-d6) δ 11.99 (s, 1H), 8.78 (d, J = 0.8 Hz, |
| | | 1H), 8.13-8.07 (m, 1H), 8.04 (ddd, J = 8.4, 6.7, 1.8 Hz, 1H), 7.66 (t, J = |
| | | 7.8 Hz, 1H), 7.25-7.16 (m, 1H), 6.35 (s, 1H), 5.99 (t, J = 6.4 Hz, |
| | | 1H), 3.38 (s, 3H), 2.66 (dt, J = 8.9, 6.0 Hz, 1H), 2.62-2.53 (m, 1H), |
| | | 2.22-2.11 (m, 3H), 2.01 (s, 3H), 1.85-1.63 (m, 2H). |
| 4.128 | 592.0 | 1H NMR (400 MHz, DMSO-d6) δ 8.78 (d, J = 0.8 Hz, 1H), 8.13- |
| | | 8.07 (m, 1H), 8.07-7.99 (m, 1H), 7.66 (t, J = 7.8 Hz, 1H), 7.48 (s, |
| | | 1H), 7.34 (d, J = 2.8 Hz, 1H), 6.27 (s, 1H), 3.38 (s, 3H), 2.59-2.52 (m, |
| | | 2H), 2.21-2.12 (m, 3H), 2.01 (s, 3H), 1.93-1.79 (m, 1H), 1.68-1.52 |
| | | (m, 1H). |
| 4.129 | 581.8 | 1H NMR (400 MHz, DMSO-d6) δ 8.80 (s, 1H), 8.72-8.61 (m, 1H), |
| | | 8.19 (dd, J = 9.2, 1.6 Hz, 1H), 8.15-8.08 (m, 1H), 8.08-8.02 (m, 1H), |
| | | 7.84 (s, 1H), 7.67 (t, J = 7.8 Hz, 1H), 7.34 (dd, J = 9.2, 4.4 Hz, 1H), |
| | | 6.42 (s, 1H), 3.39 (s, 3H), 2.95-2.85 (m, 1H), 2.74-2.68 (m, 1H), |
| | | 2.21 (s, 3H), 2.05 (s, 3H), 2.02-1.93 (m, 1H), 1.86-1.77 (m, 1H). |
| 4.130 | 593.0 | 1H NMR (400 MHz, DMSO-d6) δ 13.19 (s, 1H), 8.78 (d, J = 0.8 Hz, |
| | | 1H), 8.13-8.07 (m, 1H), 8.07-8.00 (m, 1H), 7.66 (t, J = 7.8 Hz, 1H), |
| | | 7.40 (s, 1H), 6.32 (s, 1H), 3.38 (s, 3H), 2.79 (dt, J = 8.9, 5.8 Hz, 1H), |
| | | 2.64-2.56 (m, 1H), 2.20-2.13 (m, 3H), 2.02 (s, 3H), 2.00-1.93 (m, |
| | | 1H), 1.74 (dt, J = 9.0, 5.6 Hz, 1H). |
| 4.131 | 576.0 | 1H NMR (400 MHz, DMSO-d6) δ 8.78 (d, J = 0.8 Hz, 1H), 8.09 (t, J = |
| | | 7.2 Hz, 1H), 8.04 (t, J = 6.5 Hz, 1H), 7.66 (t, J = 7.8 Hz, 1H), 7.47- |
| | | 7.38 (m, 2H), 6.27 (s, 1H), 3.38 (s, 3H), 2.54 (m, 2H), 2.22-2.14 (m, |
| | | 3H), 2.01 (s, 3H), 1.90-1.80 (m, 1H), 1.67-1.56 (m, 1H). |
| 4.132 | 617.2 | 1H NMR (400 MHz, DMSO) δ 8.79 (s, 1H), 8.10 (ddd, J = 8.2, 6.7, 1.8 |
| | | Hz, 1H), 8.05 (ddd, J = 8.3, 6.7, 1.8 Hz, 1H), 7.67 (t, J = 7.8 Hz, 1H), |
| | | 7.52 (dd, J = 7.9, 2.4 Hz, 1H), 7.18-7.10 (m, 1H), 7.06 (dd, J = 11.6, |
| | | 8.4 Hz, 1H), 6.29 (s, 1H), 5.28 (s, 1H), 2.62-2.54 (m, 1H), 2.47 (m, |
| | | 1H), 2.18 (s, 3H), 2.02 (s, 3H), 1.69 (t, J = 7.4 Hz, 2H), 1.48 (s, 6H), |
| | | 1.47 (d, J = 1.3 Hz, 3H). |
| 4.133 | 598.1 | 1H NMR (400 MHz, DMSO) δ 9.43 (s, 1H), 8.79 (s, 1H), 8.14-8.06 |
| | | (m, 1H), 8.06-8.00 (m, 2H), 7.67 (t, J = 7.8 Hz, 1H), 7.48 (t, J = 7.7 |
| | | Hz, 1H), 7.35 (dd, J = 7.5, 1.1 Hz, 1H), 6.41 (s, 1H), 3.40 (s, 3H), 3.37- |
| | | 3.27 (m, 1H), 2.93-2.82 (m, 1H), 2.19 (s, 3H), 2.11-2.00 (m, 1H), |
| | | 2.06 (s, 3H), 1.88 (dt, J = 9.1, 5.3 Hz, 1H). |
| 4.134 | 557.2 | 1H NMR (400 MHz, DMSO) δ 13.46 (s, 1H), 8.80 (s, 1H), 8.10 (t, J = |
| | | 7.2 Hz, 1H), 8.06 (t, J = 7.2 Hz, 1H), 7.91 (d, J = 6.1 Hz, 1H), 7.79 (d, J = |
| | | 7.4 Hz, 1H), 7.68 (t, J = 7.8 Hz, 1H), 7.66 (s, 1H), 6.88 (t, J = 6.8 Hz, |
| | | 1H), 6.27 (s, 1H), 3.39 (s, 3H), 2.60 (dt, J = 9.6, 5.5 Hz, 1H), 2.46 (m, |
| | | 1H), 2.20 (s, 3H), 2.03 (s, 3H), 1.74 (dt, J = 9.6, 5.5 Hz, 1H), 1.65- |
| | | 1.52 (m, 1H). |
| 4.135 | 580.8 | 1H NMR (400 MHz, DMSO-d6) δ 8.79 (d, J = 0.7 Hz, 1H), 8.67-8.61 |
| | | (m, 1H), 8.15-8.01 (m, 2H), 7.95 (s, 1H), 7.75 (dt, J = 9.0, 1.3 Hz, |
| | | 1H), 7.67 (t, J = 7.8 Hz, 1H), 7.26-7.18 (m, 1H), 6.88 (td, J = 6.8, 1.4 |
| | | Hz, 1H), 6.32 (s, 1H), 3.39 (s, 3H), 2.71-2.60 (m, 1H), 2.57-2.47 (m, |
| | | 1H), 2.21 (s, 3H), 2.04 (s, 3H), 1.77-1.67 (m, 2H). |
| 4.136 | 581.8 | 1H NMR (400 MHz, DMSO-d6) δ 9.07 (dd, J = 7.0, 1.7 Hz, 1H), 8.79 |
| | | (s, 1H), 8.53 (d, J = 4.2 Hz, 1H), 8.23 (s, 1H), 8.16-7.98 (m, 2H), 7.67 |
| | | (t, J = 7.8 Hz, 1H), 7.03 (dd, J = 7.1, 4.0 Hz, 1H), 6.32 (s, 1H), 3.39 (s, |
| | | 3H), 2.83-2.74 (m, 1H), 2.74-2.63 (m, 1H), 2.19 (s, 3H), 2.04 (s, |
| | | 3H), 2.02-1.96 (m, 1H), 1.79-1.65 (m, 1H). |
| 4.137 | 580.8 | 1H NMR (400 MHz, DMSO-d6) δ 8.80 (s, 1H), 8.60 (d, J = 7.0 Hz, |
| | | 1H), 8.17-7.95 (m, 3H), 7.67 (t, J = 7.8 Hz, 1H), 7.06 (d, J = 7.0 Hz, |
| | | 1H), 6.88 (t, J = 7.0 Hz, 1H), 6.73 (d, J = 2.3 Hz, 1H), 6.41 (s, 1H), 3.40 |
| | | (s, 3H), 2.83-2.72 (m, 1H), 2.67-2.59 (m, 1H), 2.21 (s, 3H), 2.05 (s, |
| | | 3H), 1.90-1.70 (m, 2H). |
| 4.138 | 557.9 | 1H NMR (400 MHz, DMSO-d6) δ 8.79 (d, J = 0.8 Hz, 1H), 8.15-8.00 |
| | | (m, 2H), 7.67 (t, J = 7.8 Hz, 1H), 7.32 (d, J = 6.8 Hz, 1H), 6.29-6.27 |
| | | (m, 1H), 6.25 (d, J = 1.7 Hz, 1H), 6.08 (dd, J = 6.9, 1.8 Hz, 1H), 3.39 |
| | | (s, 3H), 2.61-2.54 (m, 1H), 2.40-2.25 (m, 1H), 2.18 (s, 3H), 2.01 (s, |
| | | 3H), 1.82-1.50 (m, 2H). |
| 4.139 | 558.0 | 1H NMR (400 MHz, DMSO-d6) δ 8.79 (d, J = 0.8 Hz, 1H), 8.19-7.98 |
| | | (m, 2H), 7.67 (t, J = 7.8 Hz, 1H), 7.48-7.26 (m, 2H), 6.33 (d, J = 9.5 |
| | | Hz, 1H), 6.23 (s, 1H), 3.39 (s, 3H), 2.42-2.26 (m, 2H), 2.17 (s, 3H), |
| | | 2.01 (s, 3H), 1.67-1.49 (m, 2H). |
| 4.140 | 559.9 | 1H NMR (400 MHz, DMSO-d6) δ 8.79 (d, J = 0.9 Hz, 1H), 8.24 (d, J = |
| | | 2.5 Hz, 1H), 8.16-7.99 (m, 2H), 7.88 (td, J = 8.2, 2.7 Hz, 1H), 7.67 (t, |
| | | J = 7.8 Hz, 1H), 7.16 (dd, J = 8.5, 2.9 Hz, 1H), 6.42-6.20 (m, 1H), |
| | | 3.39 (s, 3H), 2.64-2.55 (m, 2H), 2.18 (s, 3H), 2.03 (s, 3H), 1.84-1.65 |
| | | (m, 2H). |
| 4.141 | 577.0 | 1H NMR (400 MHz, DMSO-d6) δ 8.78 (d, J = 0.8 Hz, 1H), 8.14-8.07 |
| | | (m, 1H), 8.07-8.01 (m, 1H), 7.66 (t, J = 7.8 Hz, 1H), 7.38-7.25 (m, |
| | | 1H), 7.25-7.16 (m, 1H), 7.15-7.05 (m, 1H), 6.35 (s, 1H), 3.39 (s, |
| | | 3H), 2.72-2.56 (m, 2H), 2.25-2.13 (m, 3H), 2.02 (s, 3H), 1.85-1.72 |
| | | (m, 2H). |
| 4.142 | 597.9 | 1H NMR (400 MHz, Chloroform-d) δ 8.63 (d, J = 0.8 Hz, 1H), 8.46 |
| | (M + Na)+ | (dd, J = 4.7, 1.5 Hz, 1H), 8.10 (ddd, J = 8.2, 6.6, 1.8 Hz, 1H), 7.98 (ddd, |
| | | J = 8.2, 6.7, 1.8 Hz, 1H), 7.71 (dd, J = 8.0, 1.5 Hz, 1H), 7.51 (t, J = 7.8 |
| | | Hz, 1H), 7.14 (dd, J = 8.0, 4.7 Hz, 1H), 5.93 (d, J = 1.0 Hz, 1H), 3.26 |
| | | (s, 3H), 3.07-2.92 (m, 2H), 2.27 (s, 3H), 2.06 (s, 3H), 1.60 (ddd, J = |
| | | 8.8, 6.1, 4.7 Hz, 1H). |
| 4.143 | 582.0 | 1H NMR (400 MHz, Chloroform-d) δ 8.63 (d, J = 0.8 Hz, 1H), 8.39 (dt, |
| | (M + Na)+ | J = 4.8, 1.3 Hz, 1H), 8.10 (ddd, J = 8.2, 6.5, 1.8 Hz, 1H), 7.98 (ddd, J = |
| | | 8.2, 6.6, 1.9 Hz, 1H), 7.51 (t, J = 7.8 Hz, 1H), 7.43 (ddd, J = 9.7, 8.3, |
| | | 1.4 Hz, 1H), 7.21 (ddd, J = 8.6, 4.7, 4.1 Hz, 1H), 5.91 (d, J = 1.0 Hz, |
| | | 1H), 3.25 (s, 3H), 3.07 (ddd, J = 8.9, 6.1, 4.4 Hz, 1H), 2.80 (dt, J = 9.1, |
| | | 4.7 Hz, 1H), 2.26 (s, 3H), 2.10-2.03 (m, 4H), 1.61 (ddd, J = 8.8, 6.1, |
| | | 4.8 Hz, 1H). |
| 4.144 | 597.9 | 1H NMR (400 MHz, Chloroform-d) δ 8.70-8.62 (m, 2H), 8.57 (d, J = |
| | (M + Na)+ | 5.7 Hz, 1H), 8.11 (ddd, J = 8.1, 6.5, 1.8 Hz, 1H), 7.98 (ddd, J = 8.2, 6.6, |
| | | 1.9 Hz, 1H), 7.70 (d, J = 5.7 Hz, 1H), 7.52 (t, J = 7.8 Hz, 1H), 5.89 (d, J = |
| | | 1.0 Hz, 1H), 3.25 (s, 3H), 2.74-2.66 (m, 1H), 2.58 (dt, J = 8.9, 5.9 |
| | | Hz, 1H), 2.28 (s, 3H), 2.07 (s, 3H), 1.80 (dt, J = 8.9, 6.2 Hz, 1H), 1.75 |
| | | (dt, J = 8.9, 6.1 Hz, 1H). |
| 4.145 | 598.0 | 1H NMR (400 MHz, Chloroform-d) δ 8.63 (d, J = 0.8 Hz, 1H), 8.54 (d, |
| | (M + Na)+ | J = 5.6 Hz, 1H), 8.10 (td, J = 7.2, 6.5, 1.9 Hz, 1H), 8.03-7.93 (m, 1H), |
| | | 7.58-7.46 (m, 1H), 7.40-7.27 (m, 2H), 5.91 (s, 1H), 3.25 (s, 3H), |
| | | 2.96-2.88 (m, 1H), 2.71 (dt, J = 9.9, 5.5 Hz, 1H), 2.24 (s, 3H), 2.04 (s, |
| | | 3H), 1.95 (dt, J = 9.1, 5.7 Hz, 1H), 1.69 (dt, J = 8.8, 5.9 Hz, 1H). |
| 4.146 | 582.0 | 1H NMR (400 MHz, Chloroform-d) δ 8.64 (d, J = 0.8 Hz, 1H), 8.59 (d, |
| | (M + Na)+ | J = 2.5 Hz, 1H), 8.51 (d, J = 5.4 Hz, 1H), 8.11 (ddd, J = 8.1, 6.5, 1.8 |
| | | Hz, 1H), 7.98 (ddd, J = 8.2, 6.6, 1.8 Hz, 1H), 7.52 (t, J = 7.8 Hz, 1H), |
| | | 7.29-7.23 (m, 1H), 5.89 (d, J = 1.0 Hz, 1H), 3.25 (s, 3H), 2.89-2.80 |
| | | (m, 1H), 2.68-2.59 (m, 1H), 2.26 (s, 3H), 2.07 (s, 3H), 1.88-1.77 (m, 2H). |
| 4.147 | 593.1 | 1H NMR (400 MHz, Chloroform-d) δ 8.62 (d, J = 0.8 Hz, 1H), 8.09 |
| | (M + Na)+ | (ddd, J = 8.1, 6.5, 1.8 Hz, 1H), 7.98 (ddd, J = 8.1, 6.7, 1.8 Hz, 1H), 7.50 |
| | | (t, J = 7.8 Hz, 1H), 7.40 (d, J = 2.4 Hz, 1H), 6.02 (d, J = 2.4 Hz, 1H), |
| | | 5.80 (s, 1H), 3.62-3.54 (m, 1H), 3.25 (s, 3H), 2.71-2.62 (m, 1H), |
| | | 2.58-2.48 (m, 1H), 2.24 (s, 3H), 2.01 (s, 3H), 1.67 (dt, J = 8.9, 5.8 Hz, |
| | | 1H), 1.50 (dt, J = 9.1, 5.6 Hz, 1H), 1.14-1.00 (m, 4H). |
| 4.148 | 563.0 | 1H NMR (400 MHz, DMSO-d6) δ 8.78 (d, J = 0.8 Hz, 1H), 8.13- |
| | | 8.07 (m, 1H), 8.07-8.00 (m, 1H), 7.66 (t, J = 7.8 Hz, 1H), 7.59 (d, J = |
| | | 2.5 Hz, 1H), 6.22 (s, 1H), 3.68 (s, 3H), 3.38 (s, 3H), 2.45-2.35 (m, |
| | | 1H), 2.24-2.12 (m, 4H), 1.99 (s, 3H), 1.63-1.46 (m, 2H). |
| 4.149 | 558.9 | 1H NMR (400 MHz, DMSO-d6) δ 8.77 (d, J = 0.8 Hz, 1H), 8.12-8.07 |
| | | (m, 1H), 8.07-8.00 (m, 1H), 7.66 (t, J = 7.8 Hz, 1H), 6.20 (s, 1H), |
| | | 5.93 (s, 1H), 3.64 (s, 3H), 3.38 (s, 3H), 2.53-2.36 (m, 2H), 2.20 (s, |
| | | 3H), 2.16 (s, 3H), 1.99 (s, 3H), 1.64-1.51 (m, 2H). |
| 4.150 | 542.3 | 1H NMR (400 MHz, DMSO-d6) δ 8.80 (s, 1H), 8.72 (d, J = 2.2 Hz, |
| | | 1H), 8.58 (dd, J = 5.1, 1.5 Hz, 1H), 8.14-8.08 (m, 1H), 8.08-8.02 (m, |
| | | 1H), 7.99 (d, J = 8.1 Hz, 1H), 7.67 (t, J = 7.8 Hz, 1H), 7.62 (dd, J = 8.1, |
| | | 5.0 Hz, 1H), 6.34 (s, 1H), 3.39 (s, 3H), 2.74-2.58 (m, 2H), 2.18 (s, |
| | | 3H), 2.03 (s, 3H), 1.95-1.72 (m, 2H). |
| 4.151 | 544.9 | 1H NMR (400 MHz, DMSO-d6) δ 8.79 (d, J = 0.8 Hz, 1H), 8.12-8.08 |
| | | (m, 1H), 8.08-8.01 (m, 1H), 7.66 (t, J = 7.8 Hz, 1H), 7.32 (d, J = 1.9 |
| | | Hz, 1H), 6.32 (s, 1H), 6.09 (d, J = 1.9 Hz, 1H), 3.82 (s, 3H), 3.38 (s, |
| | | 3H), 2.59-2.42 (m, 2H), 2.18 (s, 3H), 2.02 (s, 3H), 1.71 (dt, J = 8.9, |
| | | 5.3 Hz, 1H), 1.58 (dt, J = 8.7, 5.6 Hz, 1H). |
| 4.152 | 570.0 | 1H NMR (400 MHz, DMSO-d6) δ 8.78 (d, J = 0.8 Hz, 1H), 8.44 (s, |
| | | 1H), 8.13-8.07 (m, 1H), 8.04 (ddd, J = 8.3, 6.7, 1.8 Hz, 1H), 7.66 (t, J = |
| | | 7.8 Hz, 1H), 6.34 (s, 1H), 3.85 (s, 3H), 3.38 (s, 3H), 2.82-2.73 (m, |
| | | 1H), 2.59-2.55 (m, 1H), 2.18 (s, 3H), 2.01 (s, 3H), 1.75-1.68 (m, 2H). |
| 4.153 | 576.8 | 1H NMR (400 MHz, Methanol-d4) δ 8.71 (s, 1H), 8.11 (t, J = 7.2 Hz, |
| | | 1H), 8.05 (t, J = 7.0 Hz, 1H), 7.70 (t, J = 7.7 Hz, 1H), 7.62 (t, J = 7.9 |
| | | Hz, 1H), 7.36 (d, J = 7.5 Hz, 1H), 7.25 (d, J = 7.8 Hz, 1H), 6.29 (s, 1H), |
| | | 3.30 (s, 3H), 2.94 (dt, J = 9.1, 5.2 Hz, 1H), 2.64 (dt, J = 9.5, 5.1 Hz, |
| | | 1H), 2.23 (s, 3H), 2.10 (s, 3H), 1.92 (dt, J = 9.6, 5.2 Hz, 1H), 1.76- |
| | | 1.69 (m, 1H). |
| 4.154 | 542.3 | 1H NMR (400 MHz, DMSO-d6) δ 8.79 (s, 1H), 8.57-8.47 (m, 1H), |
| | | 8.10 (ddd, J = 8.1, 6.8, 1.8 Hz, 1H), 8.05 (ddd, J = 8.2, 6.7, 1.8 Hz, 1H), |
| | | 7.80 (td, J = 7.7, 1.8 Hz, 1H), 7.67 (t, J = 7.8 Hz, 1H), 7.52 (d, J = 7.8 |
| | | Hz, 1H), 7.33-7.25 (m, 1H), 6.33 (s, 1H), 3.39 (s, 3H), 2.91-2.81 (m, |
| | | 1H), 2.80-2.72 (m, 1H), 2.17 (s, 3H), 2.03 (s, 3H), 1.91-1.81 (m, |
| | | 1H), 1.79-1.69 (m, 1H). |
| 4.155 | 560.2 | 1H NMR (400 MHz, DMSO-d6) δ 8.79 (s, 1H), 8.51 (d, J = 2.9 Hz, |
| | | 1H), 8.13-8.07 (m, 1H), 8.07-8.01 (m, 1H), 7.72-7.63 (m, 2H), |
| | | 7.56 (dd, J = 8.7, 4.5 Hz, 1H), 6.31 (s, 1H), 3.39 (s, 3H), 2.79 (t, J = 7.3 |
| | | Hz, 2H), 2.17 (s, 3H), 2.02 (s, 3H), 1.82-1.76 (m, 1H), 1.75-1.67 (m, 1H). |
| 4.156 | 597.9 | 1H NMR (400 MHz, Chloroform-d) δ 8.72 (s, 1H), 8.64 (d, J = 0.8 Hz, |
| | (M + Na)+ | 1H), 8.56 (d, J = 5.5 Hz, 1H), 8. 11 (ddd, J = 8.0, 6.5, 1.8 Hz, 1H), 7.99 |
| | | (ddd, J = 8.1, 6.6, 1.8 Hz, 1H), 7.52 (t, J = 7.8 Hz, 1H), 7.24 (d, J = 5.5 |
| | | Hz, 1H), 5.92 (s, 1H), 3.25 (s, 3H), 2.82-2.72 (m, 2H), 2.27 (s, 3H), |
| | | 2.07 (s, 3H), 1.82 (dt, J = 8.7, 6.1 Hz, 1H), 1.75 (dt, J = 8.5, 6.2 Hz, 1H). |
| 4.157 | 603.0 | 1H NMR (400 MHz, Chloroform-d) δ 8.63 (d, J = 0.8 Hz, 1H), 8.10 |
| | (M + Na)+ | (ddd, J = 8.1, 6.6, 1.8 Hz, 1H), 7.98 (ddd, J = 8.2, 6.6, 1.9 Hz, 1H), 7.76 |
| | | (d, J = 2.7 Hz, 1H), 7.51 (t, J = 7.8 Hz, 1H), 7.14 (t, J = 60.8 Hz, 1H), |
| | | 6.31 (d, J = 2.7 Hz, 1H), 5.81 (d, J = 1.0 Hz, 1H), 3.25 (s, 3H), 2.79- |
| | | 2.68 (m, 1H), 2.45 (dt, J = 9.0, 5.6 Hz, 1H), 2.24 (s, 3H), 2.03 (s, 3H), |
| | | 1.80-1.70 (m, 1H), 1.54 (dt, J = 9.0, 5.6 Hz, 1H). |
| 4.158 | 567.0 | 1H NMR (400 MHz, Chloroform-d) δ 8.63 (d, J = 0.8 Hz, 1H), 8.10 |
| | (M + Na)+ | (ddd, J = 8.1, 6.5, 1.8 Hz, 1H), 7.98 (ddd, J = 8.1, 6.5, 1.8 Hz, 1H), 7.51 |
| | | (t, J = 7.8 Hz, 1H), 7.34 (d, J = 2.3 Hz, 1H), 6.09 (d, J = 2.3 Hz, 1H), |
| | | 5.85 (s, 1H), 3.94 (s, 3H), 3.25 (s, 3H), 2.66 (ddd, J = 8.9, 5.9, 4.6 Hz, |
| | | 1H), 2.60-2.51 (m, 1H), 2.24 (s, 3H), 2.03 (s, 3H), 1.67 (dt, J = 8.9, |
| | | 5.8 Hz, 1H), 1.53 (dt, J = 9.1, 5.7 Hz, 1H). |
| 4.159 | 614.1 | 1H NMR (400 MHz, Chloroform-d) δ 8.64 (d, J = 0.8 Hz, 1H), 8.11 |
| | (M + Na)+ | (ddd, J = 8.2, 6.6, 1.8 Hz, 1H), 7.98 (ddd, J = 8.2, 6.6, 1.8 Hz, 1H), 7.77 |
| | | (t, J = 7.8 Hz, 1H), 7.55-7.46 (m, 2H), 7.38-7.31 (m, 1H), 6.60 (t, J = |
| | | 55.5 Hz, 1H), 5.89 (d, J = 1.0 Hz, 1H), 3.25 (s, 3H), 2.97 (ddd, J = |
| | | 8.9, 6.1, 4.3 Hz, 1H), 2.52 (ddd, J = 8.7, 5.9, 4.3 Hz, 1H), 2.25 (s, 3H), |
| | | 2.05 (s, 3H), 2.00 (ddd, J = 9.0, 5.9, 5.0 Hz, 1H), 1.59 (ddd, J = 8.7, 6.1, |
| | | 4.9 Hz, 1H). |
| 4.160 | 611.1 | 1H NMR (400 MHz, DMSO-d6) δ 9.14 (s, 1H), 9.03 (s, 1H), 8.79 (d, J = |
| | | 0.8 Hz, 1H), 8.15-8.00 (m, 2H), 7.67 (t, J = 7.8 Hz, 1H), 6.39 (s, |
| | | 1H), 3.39 (s, 3H), 3.04-2.96 (m, 1H), 2.90-2.80 (m, 1H), 2.22-2.14 |
| | | (m, 3H), 2.03 (s, 3H), 1.97-1.83 (m, 2H). |
| 4.161 | 577.0 | 1H NMR (400 MHz, DMSO-d6) δ 8.78 (d, J = 0.8 Hz, 1H), 8.17-7.98 |
| | | (m, 2H), 7.66 (t, J = 7.8 Hz, 1H), 7.18-6.98 (m, 3H), 6.29 (s, 1H), |
| | | 3.38 (s, 3H), 2.62-2.53 (m, 2H), 2.20-2.12 (m, 3H), 2.02 (s, 3H), |
| | | 1.83-1.67 (m, 2H). |
| 4.162 | 577.0 | 1H NMR (400 MHz, DMSO-d6) δ 8.78 (d, J = 0.8 Hz, 1H), 8.16-7.99 |
| | | (m, 2H), 7.66 (t, J = 7.8 Hz, 1H), 7.33 (td, J = 8.8, 6.5 Hz, 1H), 7.25 |
| | | (ddd, J = 10.7, 9.2, 2.6 Hz, 1H), 7.08 (ddd, J = 9.0, 7.8, 2.3 Hz, 1H), |
| | | 6.33 (s, 1H), 3.38 (s, 3H), 2.63-2.52 (m, 2H), 2.25-2.10 (m, 3H), |
| | | 2.02 (s, 3H), 1.82-1.65 (m, 2H). |
| 4.163 | 577.1 | 1H NMR (400 MHz, DMSO-d6) δ 8.79 (d, J = 0.8 Hz, 1H), 8.18-7.95 |
| | | (m, 2H), 7.66 (t, J = 7.8 Hz, 1H), 7.42-7.28 (m, 1H), 7.11 (t, J = 8.3 |
| | | Hz, 2H), 6.31 (s, 1H), 3.38 (s, 3H), 2.84-2.71 (m, 1H), 2.48-2.44 (m, |
| | | 1H), 2.23-2.12 (m, 3H), 2.01 (s, 3H), 1.83-1.63 (m, 2H). |
| 4.164 | 558.9 | 1H NMR (400 MHz, DMSO-d6) δ 8.78 (d, J = 0.8 Hz, 1H), 8.23-7.90 |
| | | (m, 2H), 7.66 (t, J = 7.8 Hz, 1H), 7.38-7.11 (m, 4H), 6.34 (s, 1H), |
| | | 3.38 (s, 3H), 2.68-2.53 (m, 2H), 2.18 (s, 3H), 2.02 (s, 3H), 1.84-1.67 |
| | | (m, 2H). |
| 4.165 | 559.0 | 1H NMR (400 MHz, DMSO-d6) δ 8.78 (d, J = 0.8 Hz, 1H), 8.16-7.97 |
| | | (m, 2H), 7.66 (t, J = 7.8 Hz, 1H), 7.40-7.29 (m, 1H), 7.17-7.09 (m, |
| | | 2H), 7.07-6.99 (m, 1H), 6.29 (s, 1H), 3.38 (s, 3H), 2.63-2.52 (m, |
| | | 2H), 2.17 (s, 3H), 2.02 (s, 3H), 1.81-1.67 (m, 2H). |
| 4.166 | 546.1 | 1H NMR (400 MHz, DMSO-d6) δ 8.78 (d, J = 0.8 Hz, 1H), 8.14-8.01 |
| | | (m, 2H), 7.97 (s, 1H), 7.66 (t, J = 7.8 Hz, 1H), 6.27 (s, 1H), 4.01 (s, |
| | | 3H), 3.38 (s, 3H), 2.60 (t, J = 7.4 Hz, 2H), 2.17 (s, 3H), 2.01 (s, 3H), |
| | | 1.77-1.62 (m, 2H). |
| 4.167 | 546.0 | 1H NMR (400 MHz, DMSO-d6) δ 8.78 (s, 1H), 8.12-8.07 (m, 1H), |
| | | 8.04 (ddd, J = 8.3, 6.7, 1.8 Hz, 1H), 7.88 (s, 1H) 7.66 (t, J = 7.8 Hz, |
| | | 1H), 6.24 (s, 1H), 3.38 (s, 3H), 2.58 (ddd, J = 8.6, 5.9, 4.3 Hz, 1H), 2.48- |
| | | 2.42 (m, 1H), 2.38 (s, 3H), 2.16 (s, 3H), 1.99 (s, 3H), 1.65-1.53 (m, 2H). |
| 4.168 | 573.1 | 1H NMR (400 MHz, DMSO-d6) δ 8.77 (s, 1H), 8.12-8.07 (m, 1H), |
| | | 8.06-8.01 (m, 1H), 7.66 (t, J = 7.8 Hz, 1H), 7.28 (s, 1H), 6.17 (s, 1H), |
| | | 5.11-5.03 (m, 2H), 4.22 (t, J = 7.9 Hz, 2H), 3.38 (s, 3H), 2.38-2.30 |
| | | (m, 1H), 2.19-2.14 (m, 4H), 1.98 (s, 3H), 1.51 (t, J = 7.4 Hz, 2H). |
| 4.169 | 563.0 | 1H NMR (400 MHz, DMSO-d6) δ 8.78 (s, 1H), 8.12-8.07 (m, 1H), |
| | | 8.07-8.01 (m, 1H), 7.79 (d, J = 4.8 Hz, 1H), 7.66 (t, J = 7.8 Hz, 1H), |
| | | 6.28 (s, 1H), 3.72 (s, 3H), 3.38 (s, 3H), 2.69-2.60 (m, 2H), 2.20-2.15 |
| | | (m, 3H), 2.00 (s, 3H), 1.71-1.57 (m, 2H). |
| 4.170 | 547.9 | 1H NMR (400 MHz, DMSO-d6) δ 8.85 (s, 1H), 8.78 (d, J = 0.8 Hz, |
| | | 1H), 8.60 (s, 1H), 8.10 (ddd, J = 8.1, 6.7, 1.8 Hz, 1H), 8.04 (ddd, J = |
| | | 8.4, 6.8, 1.8 Hz, 1H), 7.66 (t, J = 7.8 Hz, 1H), 6.27 (s, 1H), 3.38 (s, 3H), |
| | | 2.69 (ddd, J = 8.8, 6.4, 4.5 Hz, 1H), 2.60-2.52 (m, 1H), 2.17 (s, 3H), |
| | | 2.01 (s, 3H), 1.81-1.69 (m, 2H). |
| 4.171 | 576.8 | 1H NMR (400 MHz, DMSO-d6) δ 8.80-8.78 (m, 2H), 8.64 (s, 1H), |
| | | 8.10 (td, J = 7.3, 6.5, 1.8 Hz, 1H), 8.05 (ddd, J = 8.4, 6.8, 1.8 Hz, 1H), |
| | | 7.66 (t, J = 7.8 Hz, 1H), 6.36 (s, 1H), 3.39 (s, 3H), 2.88 (ddd, J = 8.7, |
| | | 5.9, 4.3 Hz, 1H), 2.79 (ddd, J = 8.8, 6.3, 4.2 Hz, 1H), 2.17 (s, 3H), 2.03 |
| | | (s, 3H), 1.89-1.75 (m, 2H). |
| 4.172 | 597.8 | 1H NMR (400 MHz, Chloroform-d) δ 8.63 (d, J = 0.8 Hz, 1H), 8.56 |
| | (M + Na)+ | (dd, J = 2.5, 0.7 Hz, 1H), 8. 10 (ddd, J = 8.2, 6.6, 1.8 Hz, 1H), 7.98 (ddd, |
| | | J = 8.2, 6.6, 1.8 Hz, 1H), 7.73 (dd, J = 8.4, 2.5 Hz, 1H), 7.51 (t, J = 7.8 |
| | | Hz, 1H), 7.28-7.21 (m, 1H), 5.91 (d, J = 1.0 Hz, 1H), 3.25 (s, 3H), |
| | | 2.89 (ddd, J = 9.0, 6.2, 4.5 Hz, 1H), 2.61 (ddd, J = 8.8, 5.9, 4.5 Hz, 1H), |
| | | 2.25 (s, 3H), 2.05 (s, 3H), 1.91 (dt, J = 9.0, 5.6 Hz, 1H), 1.64 (dt, J = |
| | | 8.8, 5.8 Hz, 1H). |
| 4.173 | 541.0 | 1H NMR (400 MHz, DMSO-d6) δ 8.77 (s, 1H), 8.54 (dd, J = 5.1, 1.7 |
| | | Hz, 1H), 8.10-7.95 (m, 2H), 7.81 (td, J = 7.7, 1.8 Hz, 1H), 7.61 (t, J = |
| | | 7.8 Hz, 1H), 7.52 (d, J = 7.8 Hz, 1H), 7.35-7.25 (m, 1H), 6.32 (s, 1H), |
| | | 3.27 (s, 3H), 2.84 (ddd, J = 8.9, 6.0, 4.2 Hz, 1H), 2.75 (ddd, J = 8.8, 5.9, |
| | | 4.2 Hz, 1H), 2.16 (s, 3H), 2.02 (s, 3H), 1.84 (ddd, J = 8.8, 5.9, 4.4 Hz, |
| | | 1H), 1.74 (ddd, J = 8.8, 6.0, 4.5 Hz, 1H). |
| 4.174 | 548.0 | 1H NMR (400 MHz, DMSO-d6) δ 8.76 (s, 1H), 8.09-7.93 (m, 2H), |
| | | 7.70 (s, 1H), 7.61 (t, J = 7.8 Hz, 1H), 6.27 (s, 1H), 3.27 (s, 3H), 2.65- |
| | | 2.53 (m, 2H), 2.16 (s, 3H), 2.00 (s, 3H), 1.69 (t, J = 7.4 Hz, 2H). |
| 4.175 | 577.0 | 1H NMR (400 MHz, DMSO-d6) δ 8.77 (s, 1H), 8.48 (d, J = 2.4 Hz, |
| | | 1H), 8.09-7.91 (m, 3H), 7.61 (t, J = 7.8 Hz, 1H), 6.39 (s, 1H), 3.28 (s, |
| | | 3H), 2.88 (ddd, J = 8.8, 6.3, 1.9 Hz, 2H), 2.17 (s, 3H), 2.02 (s, 3H), 1.86- |
| | | 1.70 (m, 2H). |
| 4.176 | 559.1 | 1H NMR (400 MHz, DMSO-d6) δ 8.79 (s, 1H), 8.16-8.00 (m, 2H), |
| | | 7.98-7.73 (m, 1H), 7.67 (t, J = 7.8 Hz, 1H), 7.56-7.06 (m, 2H), 6.33 |
| | | (s, 1H), 3.39 (s, 3H), 2.89-2.70 (m, 2H), 2.17 (s, 3H), 2.03 (s, 3H), |
| | | 1.85 (d, J = 53.1 Hz, 2H). |
| 4.177 | 566.1 | 1H NMR (400 MHz, DMSO-d6) δ 8.78 (s, 1H), 8.09 (t, J = 7.3 Hz, |
| | | 1H), 8.07-8.01 (m, 1H), 7.78 (d, J = 4.7 Hz, 1H), 7.66 (t, J = 7.8 Hz, |
| | | 1H), 6.28 (s, 1H), 3.38 (s, 3H), 2.69-2.60 (m, 1H), 2.49-2.43 (m, |
| | | 1H), 2.17 (s, 3H), 2.00 (s, 3H), 1.71-1.55 (m, 2H). |
| 4.178 | 566.1 | 1H NMR (400 MHz, DMSO-d6) δ 8.79 (s, 1H), 8.10 (t, J = 7.1 Hz, |
| | | 1H), 8.04 (t, J = 7.0 Hz, 1H), 7.66 (t, J = 7.8 Hz, 1H), 7.41 (d, J = 4.3 |
| | | Hz, 1H), 6.30 (s, 1H), 3.38 (s, 3H), 2.71-2.44 (m, 2H), 2.19 (s, 3H), |
| | | 2.01 (s, 3H), 1.76-1.61 (m, 2H). |
| 4.179 | 566.1 | 1H NMR (400 MHz, DMSO-d6) δ 8.77 (s, 1H), 8.13-8.07 (m, 1H), |
| | | 8.08-8.00 (m, 1H), 7.66 (t, J = 7.8 Hz, 1H), 6.22 (s, 1H), 5.94 (d, J = |
| | | 5.7 Hz, 1H), 3.38 (s, 3H), 2.46-2.37 (m, 1H), 2.16 (s, 3H), 1.99 (s, |
| | | 3H), 1.67-1.55 (m, 2H). |
| 4.180 | 566.1 | 1H NMR (400 MHz, DMSO-d6) δ 8.79 (d, J = 0.9 Hz, 1H), 8.12-8.07 |
| | | (m, 1H), 8.04 (ddd, J = 8.3, 6.7, 1.8 Hz, 1H), 7.66 (t, J = 7.8 Hz, 1H), |
| | | 6.32 (s, 1H), 5.84 (d, J = 5.8 Hz, 1H), 3.38 (s, 3H), 2.58-2.47 (m, 2H), |
| | | 2.18 (s, 3H), 2.01 (s, 3H), 1.70 (dt, J = 8.9, 5.4 Hz, 1H), 1.64-1.56 (m, 1H). |
| 4.181 | 579.1 | 1H NMR (400 MHz, DMSO-d6) δ 8.78 (d, J = 0.8 Hz, 1H), 8.09 (t, J = |
| | | 7.2 Hz, 1H), 8.07-7.99 (m, 1H), 7.66 (t, J = 7.8 Hz, 1H), 7.49 (s, 1H), |
| | | 6.26 (s, 1H), 3.78 (s, 3H), 3.38 (s, 3H), 2.48-2.41 (m, 1H), 2.29-2.20 |
| | | (m, 1H), 2.18 (s, 3H), 2.00 (s, 3H), 1.67-1.52 (m, 2H). |
| 4.182 | 580.1 | 1H NMR (400 MHz, DMSO-d6) δ 8.78 (d, J = 0.8 Hz, 1H), 8.13- |
| | | 8.07 (m, 1H), 8.07-7.98 (m, 1H), 7.66 (t, J = 7.8 Hz, 1H), 6.34 (s, |
| | | 1H), 4.10 (s, 3H), 3.38 (s, 3H), 2.71 (ddd, J = 8.8, 5.9, 4.5 Hz, 1H), 2.54- |
| | | 2.51 (m, 1H), 2.18 (s, 3H), 2.01 (s, 3H), 1.70 (m, 2H). |
| 4.183 | 586.1 | 1H NMR (400 MHz, DMSO-d6) δ 8.79 (d, J = 0.8 Hz, 1H), 8.14- |
| | | 8.07 (m, 1H), 8.05 (ddd, J = 8.3, 6.7, 1.8 Hz, 1H), 7.67 (t, J = 7.8 Hz, |
| | | 1H), 7.36 (dd, J = 9.1, 6.9 Hz, 1H), 6.37 (s, 1H), 6.34 (dd, J = 9.1, 1.2 |
| | | Hz, 1H), 6.21 (d, J = 6.9 Hz, 1H), 4.12 (qt, J = 7.4, 3.6 Hz, 2H), 3.39 (s, |
| | | 3H), 2.68 (q, J = 7.0, 6.5 Hz, 1H), 2.60 (dt, J = 9.0, 5.6 Hz, 1H), 2.18 (s, |
| | | 3H), 2.02 (s, 3H), 1.74 (m, 2H), 1.19 (t, J = 7.0 Hz, 3H). |
| 4.184 | 575.1 | 1H NMR (400 MHz, DMSO-d6) δ 8.79 (d, J = 0.8 Hz, 1H), 8.13- |
| | | 8.07 (m, 1H), 8.07-8.01 (m, 1H), 7.66 (t, J = 7.8 Hz, 1H), 7.37 (dd, J = |
| | | 9.1, 6.9 Hz, 1H), 6.40-6.31 (m, 2H), 6.22 (d, J = 6.9 Hz, 1H), 3.38 |
| | | (s, 3H), 2.70-2.60 (m, 1H), 2.60-2.54 (m, 1H), 2.19 (s, 3H), 2.02 (s, |
| | | 3H), 1.80-1.60 (m, 2H). |
| 4.185 | 558.8 | 1H NMR (400 MHz, Methanol-d4) δ 8.73 (d, J = 0.8 Hz, 1H), 8.66 |
| | | (ddd, J = 5.5, 1.7, 0.8 Hz, 1H), 8.30 (td, J = 7.9, 1.7 Hz, 1H), 7.94 (ddd, |
| | | J = 8.1, 6.5, 1.8 Hz, 1H), 7.84-7.68 (m, 3H), 7.61 (td, J = 7.9, 0.8 Hz, |
| | | 1H), 6.37 (s, 1H), 3.08-3.00 (m, 1H), 2.86-2.77 (m, 1H), 2.43-2.39 |
| | | (m, 6H), 2.24 (s, 3H), 2.13-2.04 (m, 4H), 2.03-1.94 (m, 1H). |
| 4.186 | 620.7 | 1H NMR (400 MHz, Methanol-d4) δ 8.79 (d, J = 0.8 Hz, 1H), 8.67 (d, J = |
| | | 4.9 Hz, 1H), 8.05 (t, J = 4.9 Hz, 1H), 7.58 (s, 1H), 6.25 (s, 1H), 2.76 |
| | | (ddd, J = 8.7, 5.9, 4.5 Hz, 1H), 2.66 (s, 3H), 2.57 (ddd, J = 9.1, 6.2, 4.4 |
| | | Hz, 1H), 2.27 (s, 3H), 2.10 (s, 3H), 1.81-1.66 (m, 2H). |
| 4.187 | 579.0 | 1H NMR (400 MHz, DMSO-d6) δ 8.87-8.77 (m, 3H), 8.64 (d, J = 4.9 |
| | | Hz, 1H), 7.99 (t, J = 4.9 Hz, 1H), 6.36 (d, J = 1.0 Hz, 1H), 2.92-2.79 |
| | | (m, 2H), 2.46 (s, 3H), 2.30 (s, 3H), 2.18 (s, 3H), 2.01 (s, 3H), 1.87- |
| | | 1.75 (m, 2H). |
| 4.188 | 565.8 | 1H NMR (400 MHz, Methanol-d4) δ 8.72 (d, J = 0.8 Hz, 1H), 7.95 |
| | | (ddd, J = 8.1, 6.5, 1.8 Hz, 1H), 7.76 (ddd, J = 8.7, 7.1, 1.8 Hz, 1H), 7.62 |
| | | (td, J = 7.9, 0.9 Hz, 1H), 7.58 (s, 1H), 6.28-6.19 (s, 1H), 2.76 (ddd, J = |
| | | 8.8, 5.9, 4.5 Hz, 1H), 2.57 (ddd, J = 9.1, 6.2, 4.5 Hz, 1H), 2.44 (d, J = |
| | | 1.2 Hz, 3H), 2.43 (s, 3H), 2.26-2.22 (s, 3H), 2.08 (s, 3H), 1.77 (ddd, J = |
| | | 8.8, 6.2, 5.1 Hz, 1H), 1.71 (dt, J = 9.0, 5.5 Hz, 1H). |
| 4.189 | 567.0 | 1H NMR (400 MHz, DMSO-d6) δ 8.83 (s, 1H), 8.63 (d, J = 4.9 Hz, |
| | | 1H), 7.99 (t, J = 4.9 Hz, 1H), 7.70 (s, 1H), 6.27 (s, 1H), 2.58 (ddt, J = |
| | | 11.8, 7.3, 4.5 Hz, 2H), 2.46 (s, 3H), 2.30 (s, 3H), 2.18 (s, 3H), 1.99 (s, |
| | | 3H), 1.69 (t, J = 7.5 Hz, 2H). |
| 4.190 | 633.1 | 1H NMR (400 MHz, DMSO-d6) δ 8.70 (d, J = 0.8 Hz, 1H), 8.48 (d, J = |
| | | 2.4 Hz, 1H), 7.95 (ddd, J = 10.1, 8.9, 2.4 Hz, 1H), 7.35-7.26 (m, |
| | | 1H), 7.23-7.10 (m, 2H), 6.38 (s, 1H), 4.09 (m, 2H), 3.91 (m, 2H), |
| | | 3.51-3.37 (m, 4H), 2.93-2.82 (m, 2H), 2.15-2.08 (m, 3H), 2.00 (s, |
| | | 3H), 1.88-1.65 (m, 2H). |
| 4.191 | 604.2 | 1H NMR (400 MHz, DMSO-d6) δ 8.70 (s, 1H), 7.70 (s, 1H), 7.38- |
| | | 7.25 (m, 1H), 7.23-7.13 (m, 2H), 6.26 (s, 1H), 4.14-3.99 (m, 2H), |
| | | 3.97-3.81 (m, 2H), 3.55-3.32 (m, 4H), 2.62-2.55 (m, 2H), 2.13 (s, |
| | | 3H), 1.98 (s, 3H), 1.69 (t, J = 7.5 Hz, 2H). |
| 4.192 | 554.2 | 1H NMR (400 MHz, DMSO-d6) δ 8.74 (d, J = 0.8 Hz, 1H), 8.50 (d, J = |
| | | 4.4 Hz, 1H), 7.79-7.73 (m, 1H), 7.72-7.64 (m, 2H), 7.43 (t, J = 7.6 |
| | | Hz, 1H), 6.30-6.24 (m, 1H), 2.89-2.81 (m, 1H), 2.64-2.55 (m, 2H), |
| | | 2.15 (s, 3H), 1.99 (s, 3H), 1.74-1.63 (m, 2H), 0.74-0.66 (m, 2H), |
| | | 0.58-0.49 (m, 2H). (Expect 22, see 22) |
| 4.193 | 581.8 | 1H NMR (400 MHz, Methanol-d4) δ 8.72 (d, J = 0.8 Hz, 1H), 8.34 (d, J = |
| | | 2.4 Hz, 1H), 8.11 (s, 1H), 7.96 (ddd, J = 7.8, 6.9, 1.8 Hz, 1H), 7.84- |
| | | 7.75 (m, 1H), 7.61 (t, J = 7.7 Hz, 1H), 7.55 (ddd, J = 9.8, 8.5, 2.4 Hz, |
| | | 1H), 6.33 (d, J = 1.0 Hz, 1H), 3.90 (s, 3H), 3.00 (ddd, J = 8.9, 6.1, 4.4 |
| | | Hz, 1H), 2.87 (dt, J = 8.7, 4.3 Hz, 1H), 2.25 (s, 3H), 2.10 (s, 3H), 2.03- |
| | | 1.94 (m, 1H), 1.76 (ddd, J = 8.8, 6.1, 4.6 Hz, 1H). |
| 4.194 | 562.8 | 1H NMR (400 MHz, Methanol-d4) δ 8.72 (d, J = 0.8 Hz, 1H), 8.40 (d, J = |
| | | 2.9 Hz, 1H), 8.11 (s, 1H), 7.96 (td, J = 7.5, 6.9, 1.8 Hz, 1H), 7.80 (td, |
| | | J = 7.3, 6.7, 1.8 Hz, 1H), 7.67-7.52 (m, 3H), 6.29 (s, 1H), 3.91 (d, J = |
| | | 1.8 Hz, 3H), 2.93 (ddd, J = 8.8, 6.0, 4.3 Hz, 1H), 2.74-2.63 (m, 1H), |
| | | 2.26-2.21 (s, 3H), 2.10 (s, 3H), 1.93-1.86 (m, 1H), 1.72 (dd, J = 5.5, |
| | | 3.5 Hz, 1H). |
| 4.195 | 544.8 | 1H NMR (400 MHz, Methanol-d4) δ 8.73 (d, J = 0.8 Hz, 1H), 8.65- |
| | | 8.59 (m, 1H), 8.20 (td, J = 7.9, 1.7 Hz, 1H), 8.10 (s, 1H), 8.00-7.92 |
| | | (m, 1H), 7.80 (ddd, J = 8.4, 6.7, 1.8 Hz, 1H), 7.76-7.70 (m, 1H), 7.67- |
| | | 7.57 (m, 2H), 6.36 (d, J = 1.0 Hz, 1H), 3.91 (d, J = 1.8 Hz, 3H), 3.03 |
| | | (ddd, J = 9.1, 6.4, 4.5 Hz, 1H), 2.82-2.72 (m, 1H), 2.24 (d, J = 0.7 Hz, |
| | | 3H), 2.12 (s, 3H), 2.09-2.00 (m, 1H), 2.00-1.91 (m, 1H). |
| 4.196 | 591.1 | 1H NMR (400 MHz, DMSO-d6) δ 8.69 (d, J = 0.8 Hz, 1H), 8.48 (d, J = |
| | | 2.4 Hz, 1H), 7.94 (ddd, J = 10.1, 8.9, 2.5 Hz, 1H), 7.29 (td, J = 7.9, |
| | | 2.0 Hz, 1H), 7.21-7.05 (m, 2H), 6.37 (s, 1H), 3.27 (s, 3H), 3.27 (s, |
| | | 3H), 2.93-2.80 (m, 2H), 2.15-2.08 (m, 3H), 1.99 (s, 3H), 1.88-1.71 |
| | | (m, 2H). |
| 4.197 | 551.8 | 1H NMR (400 MHz, Methanol-d4) δ 8.72 (s, 1H), 8.10 (s, 1H), 8.03- |
| | | 7.92 (m, 1H), 7.86-7.75 (m, 1H), 7.68-7.52 (m, 2H), 6.30-6.19 (m, |
| | | 1H), 3.91 (d, J = 1.7 Hz, 3H), 2.76 (dt, J = 8.8, 5.4 Hz, 1H), 2.62-2.51 |
| | | (m, 1H), 2.24 (s, 3H), 2.09 (s, 3H), 1.77 (dt, J = 8.8, 5.8 Hz, 1H), 1.71 |
| | | (dt, J = 9.1, 5.6 Hz, 1H). |
| 4.198 | 610.1 | 1H NMR (400 MHz, Chloroform-d) δ 8.64 (s, 1H), 8.20 (s, 1H), 7.96- |
| | (M + Na)+ | 7.86 (m, 1H), 7.80-7.71 (m, 1H), 7.57-7.48 (m, 1H), 7.44 (s, 1H), |
| | | 7.19 (t, J = 57.8 Hz, 1H), 5.79 (s, 1H), 2.74 (dt, J = 8.8, 5.5 Hz, 1H), |
| | | 2.40 (dt, J = 9.0, 5.9 Hz, 1H), 2.25 (s, 3H), 2.01 (s, 3H), 1.74 (dt, J = |
| | | 8.9, 5.7 Hz, 1H), 1.55 (dt, J = 9.1, 5.6 Hz, 1H). |
| 4.199 | 562.8 | 1H NMR (400 MHz, Methanol-d4) δ 8.78 (s, 1H), 8.62 (d, J = 4.9 Hz, |
| | | 1H), 7.97 (t, J = 4.9 Hz, 1H), 7.55 (s, 1H), 7.43 (s, 1H), 6.20 (s, 1H), |
| | | 3.88 (s, 3H), 2.55 (s, 3H), 2.51 (dd, J = 8.6, 5.1 Hz, 1H), 2.41 (s, 3H), |
| | | 2.39-2.31 (m, 1H), 2.27 (s, 3H), 2.08 (s, 3H), 1.69-1.52 (m, 2H). |
| 4.200 | 560.8 | 1H NMR (400 MHz, Methanol-d4) δ 8.79 (d, J = 0.9 Hz, 1H), 8.63 (d, J = |
| | | 4.9 Hz, 2H), 8.23 (td, J = 7.9, 1.7 Hz, 1H), 7.98 (t, J = 4.9 Hz, 1H), |
| | | 7.75 (d, J = 8.1 Hz, 1H), 7.65 (ddd, J = 7.6, 5.4, 1.1 Hz, 1H), 6.37 (s, |
| | | 1H), 3.04 (ddd, J = 9.1, 6.5, 4.6 Hz, 1H), 2.79 (dt, J = 9.0, 5.4 Hz, 1H), |
| | | 2.56 (s, 3H), 2.42 (s, 3H), 2.28-2.24 (s, 3H), 2.12 (s, 3H), 2.06 (dt, J = |
| | | 9.1, 5.8 Hz, 1H), 2.01-1.93 (m, 1H). |
| 5.1 | 505.8 | 1H NMR (400 MHz, DMSO-d6) δ 8.83 (s, 1H), 8.02 (td, J = 7.5, 1.9 |
| | | Hz, 1H), 7.80 (s, 1H), 7.75 (d, J = 2.0 Hz, 1H), 7.73-7.66 (m, 2H), |
| | | 7.40 (t, J = 7.7 Hz, 1H), 7.36-7.26 (m, 2H), 7.20-7.09 (m, 2H), 6.20 |
| | | (s, 1H), 2.58-2.51 (m, 1H), 2.48-2.42 (m, 1H), 2.07 (s, 3H), 1.94 (s, |
| | | 3H), 1.67 (dd, J = 8.4, 6.2 Hz, 2H). |
| 5.2 | 576.2 | 1H NMR (400 MHz, DMSO-d6) δ 8.83 (s, 1H), 8.04 (s, 1H), 7.77 (s, |
| | | 1H), 7.53-7.47 (m, 1H), 7.43 (t, J = 7.6 Hz, 1H), 7.36-7.26 (m, 2H), |
| | | 7.15 (t, J = 8.8 Hz, 2H), 6.20 (s, 1H), 3.65 (s, 6H), 3.29 (d, J = 5.0 Hz, |
| | | 2H), 2.60-2.51 (m, 1H), 2.49-2.41 (m, 1H), 2.07 (s, 3H), 1.94 (s, |
| | | 3H), 1.77-1.61 (m, 2H). |
| 5.3 | 542.2 | 1H NMR (400 MHz, DMSO-d6) δ 8.83 (s, 1H), 8.34 (d, J = 5.0 Hz, |
| | | 1H), 8.20 (s, 1H), 8.03 (td, J = 7.6, 1.9 Hz, 1H), 7.80 (s, 1H), 7.75 (t, J = |
| | | 59.3 Hz, 1H), 7.75-7.73 (m, 1H), 7.41 (t, J = 7.7 Hz, 1H), 6.16 (s, |
| | | 1H), 2.79 (d, J = 4.6 Hz, 3H), 2.46-2.35 (m, 2H), 2.08 (s, 3H), 1.94 (s, |
| | | 3H), 1.62 (ddd, J = 8.6, 6.6, 1.8 Hz, 2H), 1.33-1.11 (m, 1H). |
| 5.4 | 598.2 | 1H NMR (400 MHz, DMSO-d6) δ 8.83 (s, 1H), 8.20 (s, 1H), 8.09- |
| | | 7.99 (m, 1H), 7.80 (s, 1H), 7.80-7.77 (m, 1H), 7.75 (t, J = 59.4 Hz, |
| | | 1H), 7.53-7.48 (m, 1H), 7.44 (t, J = 7.6 Hz, 1H), 6.16 (s, 1H), 3.81- |
| | | 3.58 (m, 3H), 3.57-3.51 (m, 2H), 3.33-3.23 (m, 2H), 2.41 (dtt, J = |
| | | 9.3, 6.8, 3.5 Hz, 2H), 2.07 (s, 3H), 1.93 (s, 3H), 1.71-1.52 (m, 2H), |
| | | 1.28-1.17 (m, 1H). |
| 5.5 | 610.2 | 1H NMR (400 MHz, DMSO-d6) δ 8.83 (d, J = 2.4 Hz, 1H), 8.20 (s, |
| | | 1H), 8.04 (td, J = 7.7, 1.8 Hz, 1H), 7.80 (s, 1H), 7.79 (d, J = 2.2 Hz, |
| | | 1H), 7.75 (t, J = 59.4 Hz, 1H), 7.59-7.47 (m, 1H), 7.47-7.37 (m, |
| | | 1H), 6.16 (s, 1H), 4.94-4.18 (m, 2H), 3.67-3.61 (m, 1H), 3.53-3.10 |
| | | (m, 2H), 2.45-2.35 (m, 2H), 2.07 (s, 3H), 1.93 (d, J = 3.2 Hz, 3H), |
| | | 1.90-1.72 (m, 2H), 1.62 (t, J = 7.4 Hz, 2H), 1.24 (s, 1H). |
| 5.6 | 604.2 | 1H NMR (400 MHz, DMSO-d6) δ 8.84 (s, 1H), 8.20 (s, 1H), 8.10 (td, J = |
| | | 7.7, 1.9 Hz, 1H), 7.85 (d, J = 2.1 Hz, 1H), 7.81 (s, 1H), 7.75 (t, J = |
| | | 59.4 Hz, 1H), 7.66 (ddd, J = 8.0, 6.2, 1.9 Hz, 1H), 7.46 (t, J = 7.7 Hz, |
| | | 1H), 6.16 (d, J = 2.7 Hz, 1H), 4.69-4.37 (m, 4H), 2.46-2.29 (m, 2H), |
| | | 2.07 (s, 3H), 1.94 (s, 3H), 1.66-1.58 (m, 2H). |
| 5.7 | 568.2 | 1H NMR (400 MHz, DMSO-d6) δ 8.83 (s, 1H), 8.44 (d, J = 4.4 Hz, |
| | | 1H), 8.20 (s, 1H), 8.03 (td, J = 7.5, 1.9 Hz, 1H), 7.85 (t, 2H), 7.76- |
| | | 7.74 (m, 1H), 7.66-7.57 (m, 1H), 7.40 (t, J = 7.7 Hz, 1H), 6.16 (s, |
| | | 1H), 2.46-2.26 (m, 2H), 2.07 (s, 3H), 1.93 (s, 3H), 1.62 (dd, J = 8.1, |
| | | 5.9 Hz, 2H), 1.32-1.13 (m, 1H), 0.75-0.63 (m, 2H), 0.63-0.44 (m, 2H). |
| 5.8 | 610.1 | 1H NMR (400 MHz, DMSO-d6) δ 8.88 (s, 1H), 8.83 (s, 1H), 8.20 (d, J = |
| | | 4.2 Hz, 1H), 8.04 (t, J = 7.5 Hz, 1H), 7.94-7.68 (m, 2H), 7.61 (t, J = |
| | | 6.8 Hz, 1H), 7.39 (t, J = 7.4 Hz, 1H), 6.17 (s, 1H), 2.44-2.35 (m, 4H), |
| | | 2.11 (s, 6H), 2.08 (s, 3H), 1.93 (s, 3H), 1.69-1.54 (m, 2H). |
| 5.9 | 560.1 | 1H NMR (400 MHz, DMSO-d6) δ 8.85 (s, 1H), 8.50-8.37 (m, 1H), |
| | | 8.20 (d, J = 1.1 Hz, 1H), 8.14 (d, J = 6.0, 1.1 Hz, 1H), 7.78 (d, J = 2.4 |
| | | Hz, 1H), 7.38-7.27 (m, 2H), 7.21-7.09 (m, 2H), 6.21 (s, 1H), 2.86 |
| | | (d, J = 4.6 Hz, 3H), 2.58-2.52 (m, 1H), 2.49-2.43 (m, 1H), 2.09 (s, |
| | | 3H), 2.01-1.92 (m, 3H), 1.73-1.62 (m, 2H). |
| 5.10 | 622.1 | 1H NMR (400 MHz, DMSO-d6) δ 8.86 (s, 1H), 8.22 (d, J = 6.1, 1.1 |
| | | Hz, 1H), 8.17 (d, J = 1.0 Hz, 1H), 7.89 (d, J = 2.6 Hz, 1H), 7.36-7.27 |
| | | (m, 2H), 7.19-7.11 (m, 2H), 6.21 (d, J = 2.2 Hz, 1H), 4.67-4.52 (m, |
| | | 4H), 2.58-2.52 (m, 1H), 2.49-2.42 (m, 1H), 2.08 (s, 3H), 2.00-1.93 |
| | | (m, 3H), 1.68 (dd, J = 8.5, 6.5 Hz, 2H). |
| 5.11 | 650.1 | 1H NMR (400 MHz, DMSO-d6) δ 8.85 (s, 1H), 8.24-8.09 (m, 2H), |
| | | 7.82 (d, J = 8.5 Hz, 1H), 7.35-7.27 (m, 2H), 7.19-7.10 (m, 2H), 6.20 |
| | | (s, 1H), 4.69 (d, J = 38.4 Hz, 2H), 4.07-3.92 (m, 1H), 3.75 (s, 1H), |
| | | 3.41 (s, 2H), 2.59-2.52 (m, 2H), 2.49-2.41 (m, 1H), 2.28 (s, 1H), |
| | | 2.08 (s, 3H), 1.95 (s, 3H), 1.74-1.62 (m, 2H). |
| 5.12 | 628.1 | 1H NMR (400 MHz, DMSO-d6) δ 8.85 (d, J = 1.7 Hz, 1H), 8.18-8.07 |
| | | (m, 2H), 7.83 (s, 1H), 7.37-7.25 (m, 2H), 7.22-7.09 (m, 2H), 6.20 (s, |
| | | 1H), 5.04-4.17 (m, 2H), 3.72-2.99 (m, 3H), 2.59-2.52 (m, 1H), |
| | | 2.48-2.43 (m, 1H), 2.08 (s, 3H), 2.02-1.85 (m, 5H), 1.80-1.62 (m, 3H). |
| 5.13 | 614.1 | 1H NMR (400 MHz, DMSO-d6) δ 8.85 (s, 1H), 8.17 (d, J = 5.9 Hz, |
| | | 1H), 8.08 (d, J = 1.0 Hz, 1H), 7.88-7.76 (m, 1H), 7.37-7.23 (m, 2H), |
| | | 7.23-7.07 (m, 2H), 6.21 (s, 1H), 4.67-4.37 (m, 2H), 2.57-2.52 (m, |
| | | 1H), 2.49-2.43 (m, 1H), 2.13-2.02 (m, 4H), 2.02-1.92 (m, 4H), |
| | | 1.68 (dd, J = 8.5, 6.5 Hz, 2H), 1.51 (d, J = 6.3 Hz, 3H), 0.82 (t, J = 6.7 |
| | | Hz, 3H). |
| 5.14 | 600.2 | 1H NMR (400 MHz, DMSO-d6) δ 8.76 (s, 1H), 7.85 (td, J = 7.4, 1.8 |
| | | Hz, 1H), 7.76 (ddd, J = 8.1, 6.5, 1.8 Hz, 1H), 7.51 (t, J = 7.7 Hz, 1H), |
| | | 7.37-7.23 (m, 2H), 7.20-7.05 (m, 2H), 6.46-6.15 (m, 1H), 4.63- |
| | | 4.44 (m, 4H), 2.59-2.53 (m, 1H), 2.49-2.45 (m, 1H), 2.16 (s, 3H), |
| | | 2.01 (s, 3H), 1.75-1.65 (m, 2H). |
| 5.15 | 580.2 | 1H NMR (400 MHz, DMSO-d6) δ 8.76 (s, 1H), 7.80 (t, J = 7.3 Hz, |
| | | 1H), 7.65 (ddd, J = 8.1, 6.4, 1.9 Hz, 1H), 7.48 (t, J = 7.6 Hz, 1H), 7.36- |
| | | 7.26 (m, 2H), 7.21-7.10 (m, 2H), 6.28 (s, 1H), 4.57-4.45 (m, 1H), |
| | | 4.31-4.23 (m, 1H), 4.20-4.09 (m, 1H), 2.59-2.54 (m, 1H), 2.50- |
| | | 2.41 (m, 1H), 2.16 (s, 3H), 2.01 (d, J = 2.7 Hz, 3H), 2.06-1.93 (m, |
| | | 1H), 1.75-1.65 (m, 2H), 1.31-1.04 (m, 2H). |
| 5.16 | 622.1 | 1H NMR (400 MHz, DMSO-d6) δ 8.76 (s, 1H), 8.20 (s, 1H), 7.85 (td, J = |
| | | 7.4, 1.8 Hz, 1H), 7.81 (s, 1H), 7.93-7.58 (t. J = 60 Hz, 1H), 7.60- |
| | | 7.45 (m, 2H), 6.24 (s, 1H), 4.53 (t, J = 12.4 Hz, 4H), 2.47-2.36 (m, |
| | | 2H), 2.16 (s, 3H), 2.00 (s, 3H), 1.71-1.60 (m, 2H). |
| 5.17 | 602.1 | 1H NMR (400 MHz, DMSO-d6) δ 8.76 (s, 1H), 8.20 (s, 1H), 7.83- |
| | | 7.77 (m, 2H), 7.75 (t, J = 59.4 Hz, 1H), 7.65 (ddd, J = 8.1, 6.4, 1.8 Hz, |
| | | 1H), 7.48 (t, J = 7.7 Hz, 1H), 6.23 (s, 1H), 4.56-4.45 (m, 1H), 4.30- |
| | | 4.21 (m, 1H), 4.15 (t, J = 8.0 Hz, 1H), 3.77 (dd, J = 10.1, 4.6 Hz, 2H), |
| | | 2.45-2.37 (m, 2H), 2.16 (s, 3H), 2.00 (d, J = 2.7 Hz, 3H), 1.71-1.60 |
| | | (m, 2H). |
| 5.18 | 642.1 | 1H NMR (400 MHz, DMSO-d6) δ 8.76 (s, 1H), 7.85 (td, J = 7.4, 1.8 |
| | | Hz, 1H), 7.79-7.59 (m, 1H), 7.51 (t, J = 7.7 Hz, 1H), 7.43-7.23 (m, |
| | | 2H), 7.20-7.04 (m, 2H), 6.28 (d, J = 2.2 Hz, 1H), 4.46-4.31 (m, 3H), |
| | | 4.28-4.21 (m, 1H), 4.21-4.10 (m, 1H), 3.04 (s, 3H), 2.63-2.40 (m, |
| | | 2H), 2.16 (s, 3H), 2.01 (d, J = 3.4 Hz, 3H), 1.83-1.55 (m, 2H). |
| 5.19 | 594.2 | 1H NMR (400 MHz, DMSO-d6) δ 8.75 (s, 1H), 7.79 (td, J = 7.3, 1.8 |
| | | Hz, 1H), 7.69-7.59 (m, 1H), 7.47 (t, J = 7.6 Hz, 1H), 7.36-7.27 (m, |
| | | 2H), 7.20-7.08 (m, 2H), 6.28 (s, 1H), 4.02 (p, J = 9.0, 8.4 Hz, 2H), |
| | | 3.93-3.66 (m, 2H), 3.52 (dd, J = 6.0, 1.9 Hz, 2H), 2.79-2.65 (m, 1H), |
| | | 2.61-2.42 (m, 2H), 2.16 (s, 3H), 2.01 (d, J = 2.4 Hz, 3H), 2.08-1.86 |
| | | (m, 1H), 1.77-1.65 (m, 2H). |
| 5.20 | 622.1 | 1H NMR (400 MHz, DMSO-d6) δ 8.75 (s, 1H), 7.85-7.71 (m, 1H), |
| | | 7.67-7.52 (m, 1H), 7.47 (t, J = 7.6 Hz, 1H), 7.36-7.24 (m, 2H), 7.22- |
| | | 7.06 (m, 2H), 6.34-6.20 (m, 1H), 4.52-4.01 (m, 2H), 3.97-3.90 |
| | | (m, 2H), 3.88 (dd, J = 9.2, 6.2 Hz, 1H), 2.67-2.40 (m, 3H), 2.16 (s, |
| | | 3H), 2.01 (d, J = 4.2 Hz, 3H), 1.84-1.61 (m, 2H), 1.04 (d, J = 1.3 Hz, |
| | | 3H), 0.98 (s, 3H). |
| 5.21 | 505.9 | 1H NMR (400 MHz, DMSO-d6) δ 8.69 (d, J = 2.6 Hz, 1H), 8.62 (s, |
| | | 1H), 7.93 (d, J = 1.6 Hz, 1H), 7.38-7.26 (m, 2H), 7.16 (td, J = 8.9, 2.3 |
| | | Hz, 2H), 6.85 (dd, J = 2.6, 1.2 Hz, 1H), 6.20 (d, J = 2.3 Hz, 1H), 3.30 |
| | | (d, J = 2.3 Hz, 3H), 3.01 (s, 3H), 2.49-2.41 (m, 2H), 2.04 (s, 3H), 1.95 |
| | | (s, 3H), 1.75-1.63 (m, 2H). |
| 5.22 | 519.9 | 1H NMR (400 MHz, DMSO-d6) δ 8.62 (s, 1H), 7.93 (s, 1H), 7.68 (s, |
| | | 1H), 7.38-7.26 (m, 2H), 7.24-7.08 (m, 2H), 6.19 (s, 1H), 3.82 (s, |
| | | 3H), 3.29 (s, 3H), 3.01 (s, 3H), 2.48 (m, 2H), 2.00 (s, 3H), 1.92 (s, 3H), |
| | | 1.68 (t, J = 7.5 Hz, 2H). |
| 5.23 | 491.9 | 1H NMR (400 MHz, DMSO-d6) δ 8.63 (s, 1H), 7.96 (s, 1H), 7.85 (s, |
| | | 1H), 7.78 (s, 1H), 7.51 (s, 1H), 7.38-7.28 (m, 2H), 7.20-7.09 (m, |
| | | 2H), 6.21 (s, 1H), 4.01 (s, 3H), 2.47 (m, 2H) 2.02 (s, 3H), 1.94 (s, 3H), |
| | | 1.69 (t, J = 7.5 Hz, 2H). |
| 5.24 | 605.2 | 1H NMR (400 MHz, DMSO-d6) δ 8.83 (s, 1H), 8.56 (d, J = 2.0 Hz, |
| | | 1H), 8.49 (d, J = 2.3 Hz, 1H), 8.08-7.97 (m, 1H), 7.89-7.83 (m, 1H), |
| | | 7.80 (d, J = 11.4, 1.8 Hz, 1H), 7.54-7.45 (m, 1H), 7.42 (t, J = 7.6, 1.0 |
| | | Hz, 1H), 6.24 (s, 1H), 3.62-3.50 (m, 1H), 3.40-3.28 (m, 1H), 3.26 (s, |
| | | 1H), 3.01 (d, J = 4.1 Hz, 1H), 2.66-2.53 (m, 2H), 2.07 (s, 3H), 1.94 (s, |
| | | 3H), 1.88-1.61 (m, 4H), 1.09 (s, 3H), 0.99 (d, J = 2.5 Hz, 3H). |
| 5.25 | 607.2 | 1H NMR (400 MHz, DMSO-d6) δ 8.83 (s, 1H), 8.56 (d, J = 1.9 Hz, |
| | | 1H), 8.48 (d, J = 2.3 Hz, 1H), 8.06-7.97 (m, 1H), 7.86 (t, J = 2.1 Hz, |
| | | 1H), 7.79 (dd, J = 6.4, 1.8 Hz, 1H), 7.52-7.45 (m, 1H), 7.42 (t, J = 7.6 |
| | | Hz, 1H), 6.24 (s, 1H), 3.58 (t, J = 8.0 Hz, 1H), 3.48 (d, J = 11.5 Hz, |
| | | 1H), 3.30 (d, J = 12.0 Hz, 1H), 3.26-3.03 (m, 1H), 2.64-2.53 (m, |
| | | 2H), 2.07 (s, 3H), 1.94 (s, 3H), 1.89-1.70 (m, 4H), 1.38-1.16 (m, 3H). |
| 5.26 | 520.0 | 1H NMR (400 MHz, DMSO-d6) δ 8.73 (s, 1H), 7.84 (s, 1H), 7.61 (s, |
| | | 1H), 7.39-7.25 (m, 2H), 7.25-7.06 (m, 2H), 6.21 (s, 1H), 4.00 (s, |
| | | 3H), 3.14 (s, 3H), 3.04 (s, 3H), 2.47 (m, 2H), 2.04 (s, 3H), 1.95 (s, 3H), |
| | | 1.75-1.61 (m, 2H). |
| 5.27 | 621.1 | 1H NMR (400 MHz, DMSO-d6) δ 8.83 (s, 1H), 8.56 (d, J = 1.9 Hz, |
| | | 1H), 8.48 (d, J = 2.3 Hz, 1H), 8.00 (s, 1H), 7.86 (t, J = 2.2 Hz, 1H), 7.82- |
| | | 7.70 (m, 1H), 7.53-7.29 (m, 2H), 6.23 (s, 1H), 3.48 (d, J = 27.5 Hz, |
| | | 2H), 3.12 (d, J = 37.8 Hz, 3H), 2.64-2.53 (m, 2H), 2.07 (d, J = 2.4 Hz, |
| | | 3H), 1.94 (s, 3H), 1.88-1.71 (m, 2H), 1.71-1.30 (m, 3H), 1.06 (d, J = |
| | | 73.8 Hz, 3H). |
| 5.28 | 621.2 | 1H NMR (400 MHz, DMSO-d6) δ 8.83 (s, 1H), 8.56 (d, J = 1.9 Hz, |
| | | 1H), 8.48 (d, J = 2.3 Hz, 1H), 8.02 (s, 1H), 7.87 (t, J = 2.2 Hz, 1H), 7.81- |
| | | 7.67 (m, 1H), 7.53-7.35 (m, 2H), 6.24 (s, 1H), 3.57-2.92 (m, 4H), |
| | | 2.66-2.53 (m, 3H), 2.07 (s, 3H), 1.94 (d, J = 3.0 Hz, 3H), 1.90-1.68 |
| | | (m, 2H), 1.68-1.29 (m, 3H), 1.06 (d, J = 73.7 Hz, 3H). |
| 5.29 | 617.2 | 1H NMR (400 MHz, DMSO-d6) δ 8.83 (s, 1H), 8.57-8.52 (m, 1H), |
| | | 8.48 (d, J = 2.3 Hz, 1H), 8.02 (t, J = 7.7, 1.9 Hz, 1H), 7.91-7.82 (m, |
| | | 1H), 7.82-7.74 (m, 1H), 7.55-7.46 (m, 1H), 7.42 (td, J = 7.6, 1.3 Hz, |
| | | 1H), 6.24 (s, 1H), 4.61 (s, 1H), 3.76 (s, 1H), 2.66-2.53 (m, 2H), 2.06 |
| | | (s, 3H), 2.01-1.88 (m, 5H), 1.88-1.64 (m, 6H), 1.61-1.36 (m, 4H). |
| 5.30 | 614.2 | 1H NMR (400 MHz, DMSO-d6) δ 8.76 (s, 1H), 7.82 (tt, J = 7.3, 1.6 |
| | | Hz, 1H), 7.77-7.57 (m, 1H), 7.51 (td, J = 7.6, 4.2 Hz, 1H), 7.40-7.28 |
| | | (m, 2H), 7.21-7.06 (m, 2H), 6.27 (s, 1H), 3.93 (t, J = 13.2 Hz, 1H), |
| | | 3.88-3.63 (m, 2H), 3.51 (t, J = 7.3 Hz, 1H), 2.60-2.37 (m, 2H), 2.16 |
| | | (s, 3H), 2.00 (s, 3H), 2.04-1.95 (m, 1H), 1.71 (t, J = 7.5 Hz, 2H). |
| 5.31 | 594.2 | 1H NMR (400 MHz, DMSO-d6) δ 8.75 (d, J = 0.9 Hz, 1H), 7.77 (td, J = |
| | | 7.1, 6.7, 1.5 Hz, 1H), 7.64-7.53 (m, 1H), 7.48 (t, J = 7.6 Hz, 1H), |
| | | 7.38-7.26 (m, 2H), 7.21-6.88 (m, 2H), 6.35-6.18 (m, 1H), 4.34 (p, |
| | | J = 3.7, 3.0 Hz, 0.5H, rotamer), 4.24 (tt, J = 4.7, 2.5 Hz, 0.5H, rotamer), |
| | | 3.63-3.49 (m, 1H), 3.48-3.31 (m, 1H), 3.25 (ddd, J = 10.3, 8.4, 3.1 |
| | | Hz, 0.5H, rotamer), 3.04 (d, J = 10.8 Hz, 0.5H, rotamer), 2.59-2.54 |
| | | (m, 1H), 2.54-2.37 (m, 2H), 2.16 (s, 3H), 2.00 (s, 3H), 1.99 (s, 1H), |
| | | 2.00-1.87 (m, 0.5H, rotamer), 1.88-1.76 (m, 0.5H, rotamer), 1.74- |
| | | 1.67 (m, 2H). |
| 5.32 | 594.1 | 1H NMR (400 MHz, DMSO-d6) δ 8.76 (s, 1H), 7.77 (t, J = 7.2 Hz, |
| | | 1H), 7.59 (td, J = 7.0, 6.3, 1.8 Hz, 1H), 7.48 (t, J = 7.6 Hz, 1H), 7.37- |
| | | 7.25 (m, 2H), 7.19-7.05 (m, 2H), 6.27 (d, J = 1.9 Hz, 1H), 4.38-4.31 |
| | | (m, 0.5H, rotamer), 4.28-4.21 (m, 0.5H, rotamer), 3.57 (dd, J = 9.1, |
| | | 5.3 Hz, 1H), 3.46-3.34 (m, 1H), 3.25 (td, J = 10.1, 9.3, 2.8 Hz, 0.5H, |
| | | rotamer), 3.04 (d, J = 10.9 Hz, 0.5H, rotamer), 2.61-2.41 (m, 2H), |
| | | 2.16 (s, 3H), 2.00 (s, 3H), 2.04-1.98 (m, 1H), 1.98-1.88 (m, 0.5H, |
| | | rotamer), 1.90-1.75 (m, 0.5H, rotamer), 1.74-1.67 (m, 2H). |
| 5.33 | 557.1 | 1H NMR (400 MHz, DMSO-d6) δ 8.76 (s, 1H), 8.59-8.51 (m, 1H), |
| | | 8.48 (d, J = 2.3 Hz, 1H), 8.34 (d, J = 4.3 Hz, 1H), 7.90-7.82 (m, 1H), |
| | | 7.70-7.61 (m, 1H), 6.24 (s, 1H), 3.20-2.91 (m, 6H), 2.65-2.53 (m, |
| | | 2H), 2.04 (s, 3H), 1.94 (s, 3H), 1.90-1.69 (m, 2H). |
| 5.34 | 520.9 | 1H NMR (400 MHz, Methanol-d4) δ 8.83 (s, 1H), 8.56-8.42 (m, 2H), |
| | | 8.12-8.01 (m, 1H), 7.87-7.69 (m, 3H), 7.44 (t, J = 7.7 Hz, 1H), 6.30 |
| | | (s, 1H), 2.96 (s, 3H), 2.83-2.73 (m, 1H), 2.70-2.60 (m, 1H), 2.18 (s, |
| | | 3H), 2.08 (s, 3H), 1.91-1.79 (m, 2H). |
| 5.35 | 589.2 | 1H NMR (400 MHz, DMSO-d6) δ 8.76 (s, 1H), 7.83 (td, J = 7.3, 1.8 |
| | | Hz, 1H), 7.77-7.54 (m, 1H), 7.50 (t, J = 7.7 Hz, 1H), 7.39-7.28 (m, |
| | | 2H), 7.24-7.06 (m, 2H), 6.28 (s, 1H), 4.35 (t, J = 9.6 Hz, 1H), 4.29- |
| | | 4.16 (m, 2H), 3.96-3.78 (m, 1H), 2.61-2.35 (m, 2H), 2.16 (s, 3H), |
| | | 2.06-1.94 (m, 1H), 2.01 (s, 3H), 1.77-1.65 (m, 2H). |
| 5.36 | 603.2 | 1H NMR (400 MHz, DMSO-d6) δ 8.76 (s, 1H), 7.81 (tdd, J = 7.3, 5.2, |
| | | 1.8 Hz, 1H), 7.68-7.55 (m, 1H), 7.50 (td, J = 7.6, 2.6 Hz, 1H), 7.36- |
| | | 7.25 (m, 2H), 7.21-7.09 (m, 2H), 6.27 (s, 1H), 3.93-3.66 (m, 1H), |
| | | 3.67-3.50 (m, 2H), 3.49-3.43 (m, 1H), 3.41-3.33 (m, 1H), 2.61- |
| | | 2.42 (m, 2H), 2.39-2.18 (m, 2H), 2.16 (s, 3H), 2.00 (d, J = 1.5 Hz, |
| | | 3H), 1.71 (t, J = 7.6 Hz, 2H). |
| 5.37 | 603.3 | 1H NMR (400 MHz, DMSO-d6) δ 8.76 (s, 1H), 7.85-7.72 (m, 1H), |
| | | 7.69-7.59 (m, 1H), 7.50 (td, J = 7.6, 2.7 Hz, 1H), 7.38-7.26 (m, 2H), |
| | | 7.19-7.09 (m, 2H), 6.27 (s, 1H), 3.88-3.66 (m, 1H), 3.66-3.51 (m, |
| | | 2H), 3.51-3.42 (m, 1H), 3.41-3.31 (m, 1H), 2.60-2.43 (m, 2H), |
| | | 2.37-2.18 (m, 2H), 2.16 (s, 3H), 2.00 (s, 3H), 1.70 (t, J = 4.6 Hz, 2H). |
| 5.38 | 597.2 | 1H NMR (400 MHz, DMSO-d6) δ 8.76 (s, 1H), 8.56 (d, J = 1.9 Hz, |
| | | 1H), 8.49 (d, J = 2.3 Hz, 1H), 7.87 (t, J = 2.1 Hz, 1H), 7.83-7.75 (m, |
| | | 1H), 7.65 (ddd, J = 8.0, 6.4, 1.9 Hz, 1H), 7.48 (t, J = 7.7 Hz, 1H), 6.32 |
| | | (s, 1H), 4.51 (tt, J = 6.9, 4.5 Hz, 1H), 4.30-4.21 (m, 1H), 4.20-4.09 |
| | | (m, 1H), 3.85-3.70 (m, 2H), 2.65-2.55 (m, 2H), 2.16 (s, 3H), 2.01 (d, |
| | | J = 2.7 Hz, 3H), 1.92-1.74 (m, 2H). |
| 5.39 | 611.2 | 1H NMR (400 MHz, DMSO-d6) δ 8.76 (s, 1H), 8.56 (d, J = 1.9 Hz, |
| | | 1H), 8.49 (d, J = 2.3 Hz, 1H), 7.87 (t, J = 2.1 Hz, 1H), 7.84-7.76 (m, |
| | | 1H), 7.70-7.60 (m, 1H), 7.48 (t, J = 7.7 Hz, 1H), 6.32 (s, 1H), 3.98- |
| | | 3.77 (m, 4H), 2.66-2.55 (m, 2H), 2.16 (s, 3H), 2.01 (d, J = 4.2 Hz, |
| | | 3H), 1.92-1.74 (m, 2H), 1.38 (d, J = 2.1 Hz, 3H). |
| 5.40 | 611.2 | 1H NMR (400 MHz, DMSO-d6) δ 8.76 (s, 1H), 8.56 (d, J = 1.9 Hz, |
| | | 1H), 8.49 (d, J = 2.3 Hz, 1H), 7.87 (t, J = 2.1 Hz, 1H), 7.79 (td, J = 7.3, |
| | | 1.8 Hz, 1H), 7.65 (ddd, J = 8.2, 6.4, 1.8 Hz, 1H), 7.48 (t, J = 7.6 Hz, |
| | | 1H), 6.32 (t, J = 1.2 Hz, 1H), 4.09-3.96 (m, 2H), 3.79 (dd, J = 10.0, |
| | | 5.5 Hz, 1H), 3.76-3.70 (m, 1H), 3.52 (dd, J = 6.0, 1.8 Hz, 2H), 2.79- |
| | | 2.65 (m, 1H), 2.65-2.55 (m, 2H), 2.16 (s, 3H), 2.01 (d, J = 2.3 Hz, |
| | | 3H), 1.92-1.74 (m, 2H). |
| 5.41 | 540.9 | 1H NMR (400 MHz, Methanol-d4) δ 8.83 (s, 1H), 8.54-8.44 (m, 2H), |
| | | 8.05 (td, J = 7.6, 2.1 Hz, 1H), 7.81-7.73 (m, 2H), 7.54-7.41 (m, 2H), |
| | | 6.29 (d, J = 1.0 Hz, 1H), 2.82-2.73 (m, 1H), 2.69-2.61 (m, 1H), 2.18 |
| | | (s, 3H), 2.07 (d, J = 0.9 Hz, 3H), 1.89-1.80 (m, 2H). |
| 5.42 | 556.9 | 1H NMR(400 MHz, Methanol-d4) δ 8.87-8.75 (m, 1H), 8.56-8.39 |
| | | (m, 2H), 8.12-7.98 (m, 1H), 7.88-7.78 (m, 1H), 7.78-7.69 (m, 1H), |
| | | 7.54-7.39 (m, 2H), 6.28 (s, 1H), 2.80-2.69 (m, 1H), 2.64-2.50 (m, |
| | | 1H), 2.18 (s, 3H), 2.06 (s, 3H), 1.81 (t, J = 7.5 Hz, 2H). |
| 5.43 | 557.0 | 1H NMR (400 MHz, DMSO-d6) δ 8.69 (s, 1H), 8.58-8.50 (m, 1H), |
| | | 8.50-8.41 (m, 1H), 7.92-7.78 (m, 2H), 7.72-7.58 (m, 1H), 6.23 (s, |
| | | 1H), 3.02-2.92 (m, 6H), 2.54 (s, 2H), 2.02 (s, 3H), 1.94 (s, 3H), 1.87- |
| | | 1.70 (m, 2H). |
| 5.44 | 581.2 | 1H NMR (400 MHz, DMSO-d6) δ 8.74 (d, J = 0.9 Hz, 1H), 8.56 (d, J = |
| | | 1.9 Hz, 1H), 8.53-8.47 (m, 2H), 7.87 (t, J = 2.1 Hz, 1H), 7.76 (td, J = |
| | | 7.3, 1.8 Hz, 1H), 7.72-7.63 (m, 1H), 7.44 (t, J = 7.7 Hz, 1H), 6.32 (d, |
| | | 1H), 2.89-2.80 (m, 1H), 2.65-2.56 (m, 2H), 2.16 (s, 3H), 2.01 (s, |
| | | 3H), 1.90-1.83 (m, 1H), 1.83-1.73 (m, 1H), 0.74-0.66 (m, 2H), |
| | | 0.57-0.51 (m, 2H). |
| 5.45 | 555.2 | 1H NMR (400 MHz, DMSO-d6) δ 8.75 (d, J = 0.9 Hz, 1H), 8.56 (d, J = |
| | | 1.9 Hz, 1H), 8.49 (d, J = 2.3 Hz, 1H), 8.41-8.30 (m, 1H), 7.87 (t, J = |
| | | 2.1 Hz, 1H), 7.81-7.71 (m, 2H), 7.46 (t, J = 7.7 Hz, 1H), 6.32 (d, J = |
| | | 1.0 Hz, 1H), 2.79 (d, J = 4.6 Hz, 3H), 2.66-2.55 (m, 2H), 2.16 (s, 3H), |
| | | 2.02 (s, 3H), 1.89-1.83 (m, 1H), 1.82-1.76 (m, 1H). |
| 5.46 | 657.1 | 1H NMR (400 MHz, DMSO-d6) δ 8.76 (d, J = 0.8 Hz, 1H), 7.90 (d, J = |
| | | 8.0 Hz, 1H), 7.82 (td, J = 7.3, 1.8 Hz, 1H), 7.71-7.56 (m, 1H), 7.49 (t, |
| | | J = 7.7 Hz, 1H), 7.38-7.22 (m, 2H), 7.22-7.06 (m, 2H), 6.33-6.14 |
| | | (m, 1H), 4.44-4.33 (m, 1H), 4.33-4.19 (m, 2H), 4.00-3.89 (m, 2H), |
| | | 2.90 (s, 3H), 2.58-2.43 (m, 1H), 2.16 (s, 3H), 2.01 (s, 3H), 2.05-1.94 |
| | | (m, 1H), 1.71 (ddd, J = 8.3, 6.5, 2.0 Hz, 2H). |
| 5.47 | 636.2 | 1H NMR (400 MHz, DMSO-d6) δ 8.75 (s, 1H), 7.80 (td, J = 7.3, 1.8 |
| | | Hz, 1H), 7.71-7.60 (m, 1H), 7.48 (t, J = 7.6 Hz, 1H), 7.38-7.26 (m, |
| | | 2H), 7.21-7.01 (m, 2H), 6.66 (d, J = 7.2 Hz, 1H), 6.28 (s, 1H), 4.46- |
| | | 4.35 (m, 2H), 4.30-4.22 (m, 1H), 4.21-4.14 (m, 1H), 3.87-3.79 (m, |
| | | 2H), 2.53 (d, J = 1.2 Hz, 3H), 2.16 (s, 3H), 2.00 (s, 3H), 1.78-1.61 (m, 2H). |
| 5.48 | 523.9 | 1H NMR (400 MHz, Methanol-d4) δ 8.83 (s, 1H), 8.55-8.46 (m, 2H), |
| | | 8.10-8.02 (m, 1H), 7.84-7.74 (m, 3H), 7.44 (t, J = 7.7 Hz, 1H), 6.30 |
| | | (s, 1H), 2.83-2.74 (m, 1H), 2.71-2.61 (m, 1H), 2.18 (s, 3H), 2.08 (s, |
| | | 3H), 1.92-1.79 (m, 2H). |
| 5.49 | 560.9 | 1H NMR (400 MHz, Methanol-d4) δ 8.83 (s, 1H), 8.55-8.50 (m, 1H), |
| | | 8.47 (d, J = 2.6 Hz, 1H), 8.08-8.00 (m, 1H), 7.81-7.73 (m, 2H), 7.56- |
| | | 7.41 (m, 2H), 6.29 (s, 1H), 3.15 (s, 3H), 2.93-2.85 (m, 1H), 2.82- |
| | | 2.74 (m, 1H), 2.70-2.62 (m, 1H), 2.18 (s, 3H), 2.07 (s, 3H), 1.92- |
| | | 1.78 (m, 2H), 0.65-0.49 (m, 4H). |
| 5.50 | 581.9 | 1H NMR (400 MHz, Methanol-d4) δ 8.69 (d, J = 0.8 Hz, 1H), 8.48 (t, J = |
| | | 1.6 Hz, 1H), 8.43 (d, J = 2.5 Hz, 1H), 7.83-7.75 (m, 1H), 7.74- |
| | | 7.65 (m, 1H), 7.61-7.53 (m, 1H), 7.48 (t, J = 7.6 Hz, 1H), 6.32 (s, |
| | | 1H), 2.95-2.82 (m, 1H), 2.81-2.72 (m, 1H), 2.70-2.60 (m, 1H), |
| | | 2.24 (s, 3H), 2.11 (s, 3H), 1.91-1.78 (m, 2H), 0.65-0.50 (m, 4H). |
| 5.51 | 613.3 | 1H NMR (400 MHz, DMSO-d6) δ 8.76 (d, J = 2.4 Hz, 2H), 8.68-8.62 |
| | | (m, 1H), 7.92 (s, 1H), 7.80 (tt, J = 7.3, 1.6 Hz, 1H), 7.66 (ddd, J = 8.0, |
| | | 6.4, 1.9 Hz, 1H), 7.48 (t, J = 7.7 Hz, 1H), 7.14 (t, J = 55.2 Hz, 1H), 6.34 |
| | | (s, 1H), 4.56-4.45 (m, 1H), 4.30-4.21 (m, 1H), 4.19-4.12 (m, 1H), |
| | | 3.82-3.73 (m, 2H), 2.71-2.65 (m, 1H), 2.65-2.58 (m, 1H), 2.16 (s, |
| | | 3H), 2.02 (d, J = 2.7 Hz, 3H), 1.92-1.77 (m, 2H). |
| 5.52 | 571.3 | 1H NMR (400 MHz, DMSO-d6) δ 8.77-8.76 (m, 1H), 8.76-8.74 (m, |
| | | 1H), 8.70-8.57 (m, 1H), 8.44-8.26 (m, 1H), 7.91 (s, 1H), 7.83-7.72 |
| | | (m, 2H), 7.46 (t, J = 7.7 Hz, 1H), 7.14 (t, J = 55.3 Hz, 1H), 6.34 (s, 1H), |
| | | 2.79 (d, J = 4.6 Hz, 3H), 2.71-2.64 (m, 1H), 2.64-2.57 (m, 1H), 2.16 |
| | | (s, 3H), 2.02 (s, 3H), 1.90-1.84 (m, 1H), 1.84-1.76 (m, 1H). |
| 5.53 | 620.2 | 1H NMR (400 MHz, DMSO-d6) δ 8.75 (s, 1H), 7.83-7.71 (m, 1H), |
| | | 7.75-7.59 (m, 1H), 7.56-7.43 (m, 1H), 7.32 (m, 2H), 7.23-7.03 (m, |
| | | 2H), 6.28 (d, J = 2.4 Hz, 1H), 4.21-3.99 (m, 2H), 3.95-3.80 (m, 1H), |
| | | 3.60-3.50 (m, 3H), 3.44-3.33 (m, 3H), 3.34-3.18 (m, 1H), 2.61- |
| | | 2.40 (m, 2H), 2.16 (s, 3H), 2.01 (d, J = 4.5 Hz, 3H), 1.80-1.57 (m, 2H). |
| 5.54 | 630.2 | 1H NMR (400 MHz, DMSO-d6) δ 8.76 (d, J = 0.8 Hz, 1H), 7.82 (td, J = |
| | | 7.3, 1.8 Hz, 1H), 7.70 (ddd, J = 8.1, 6.5, 1.8 Hz, 1H), 7.49 (t, J = 7.7 |
| | | Hz, 1H), 7.37-7.28 (m, 2H), 7.18-7.11 (m, 2H), 6.76 (t, J = 76 Hz, |
| | | 1H), 6.28 (d, J = 1.0 Hz, 1H), 5.03 (tt, J = 7.0, 4.1 Hz, 1H), 4.47-4.37 |
| | | (m, 1H), 4.37-4.28 (m, 1H), 4.11-3.91 (m, 2H), 2.60-2.42 (m, 2H), |
| | | 2.16 (s, 3H), 2.01 (s, 3H), 1.78-1.63 (m, 2H). |
| 5.55 | 595.1 | 1H NMR (400 MHz, DMSO-d6) δ 8.75 (s, 1H), 8.55 (d, J = 1.9 Hz, |
| | | 1H), 8.48 (d, J = 2.3 Hz, 1H), 7.86 (t, J = 2.1 Hz, 1H), 7.78 (t, J = 7.3 |
| | | Hz, 1H), 7.70-7.59 (m, 1H), 7.47 (td, J = 7.6, 3.1 Hz, 1H), 6.32 (s, |
| | | 1H), 4.61-4.37 (m, 1H), 4.05-3.73 (m, 2H), 2.69-2.56 (m, 2H), |
| | | 2.45-2.32 (m, 1H), 2.15 (s, 3H), 2.01 (s, 3H), 1.90-1.75 (m, 3H), |
| | | 1.46 (d, J = 6.3 Hz, 1.5H (amide rotomer)), 0.96 (d, J = 6.2 Hz, 1.5H |
| | | (amide rotomer)). |
| 5.56 | 609.2 | 1H NMR (400 MHz, DMSO-d6) δ 8.75 (d, J = 0.8 Hz, 1H), 8.56 (d, J = |
| | | 1.9 Hz, 1H), 8.48 (d, J = 2.3 Hz, 1H), 7.86 (t, J = 2.1 Hz, 1H), 7.84- |
| | | 7.72 (m, 1H), 7.70-7.39 (m, 2H), 6.32 (s, 1H), 4.56-4.30 (m, 2H), |
| | | 2.66-2.56 (m, 2H), 2.15 (s, 3H), 2.08-1.90 (m, 5H), 1.90-1.66 (m, |
| | | 2H), 1.45 (d, J = 6.3 Hz, 3H), 0.91 (d, J = 6.2 Hz, 3H). |
| 5.57 | 607.0 | 1H NMR (400 MHz, DMSO-d6) δ 8.75 (s, 1H), 8.55 (d, J = 1.9 Hz, |
| | | 1H), 8.48 (d, J = 2.3 Hz, 1H), 7.86 (t, J = 2.2 Hz, 1H), 7.81-7.72 (m, |
| | | 1H), 7.67-7.37 (m, 2H), 6.32 (s, 1H), 4.03-3.95 (m, 2H), 2.66-2.54 |
| | | (m, 2H), 2.48-2.39 (m, 2H), 2.15 (s, 3H), 2.01 (s, 3H), 1.91-1.75 (m, |
| | | 2H), 1.67-1.54 (m, 2H), 0.71-0.60 (m, 2H). |
| 5.58 | 622.0 | 1H NMR (400 MHz, DMSO-d6) δ 8.76 (s, 1H), 8.55 (d, J = 2.0 Hz, |
| | | 1H), 8.49 (d, J = 2.3 Hz, 1H), 7.92-7.80 (m, 2H), 7.72 (t, J = 7.0 Hz, |
| | | 1H), 7.52 (t, J = 7.7 Hz, 1H), 6.31 (s, 1H), 4.81-4.56 (m, 2H), 4.27- |
| | | 4.04 (m, 2H), 3.87 (t, J = 7.4 Hz, 2H), 2.71-2.53 (m, 4H), 2.16 (s, |
| | | 3H), 2.00 (s, 3H), 1.91-1.67 (m, 2H). |
| 5.59 | 649.1 | 1H NMR (400 MHz, DMSO-d6) δ 8.76 (s, 1H), 8.55 (d, J = 1.9 Hz, |
| | | 1H), 8.48 (d, J = 2.3 Hz, 1H), 7.89-7.83 (m, 2H), 7.75-7.67 (m, 1H), |
| | | 7.57-7.44 (m, 1H), 6.32 (s, 1H), 5.19-4.81 (m, 1H), 4.19-4.04 (m, |
| | | 1H), 3.97-3.85 (m, 1H), 2.71-2.55 (m, 3H), 2.36-2.22 (m, 1H), |
| | | 2.16 (s, 3H), 2.01 (s, 3H), 1.93-1.71 (m, 2H). |
| 5.60 | 585.1 | 1H NMR (400 MHz, DMSO-d6) δ 8.74 (s, 1H), 8.55 (d, J = 1.9 Hz, |
| | | 1H), 8.48 (d, J = 2.3 Hz, 1H), 8.40-8.32 (m, 1H), 7.86 (t, J = 2.2 Hz, |
| | | 1H), 7.81-7.68 (m, 2H), 7.45 (t, J = 7.7 Hz, 1H), 6.31 (s, 1H), 3.53- |
| | | 3.47 (m, 2H), 3.32 (q, J = 6.0 Hz, 2H), 2.64-2.56 (m, 2H), 2.15 (s, |
| | | 3H), 2.01 (s, 3H), 1.88-1.72 (m, 2H). |
| 5.61 | 558.2 | 1H NMR (400 MHz, DMSO-d6) δ 8.75 (d, J = 0.8 Hz, 1H), 8.56 (d, J = |
| | | 1.9 Hz, 1H), 8.49 (d, J = 2.3 Hz, 1H), 8.34 (s, 1H), 7.86 (t, J = 2.1 Hz, |
| | | 1H), 7.81-7.77 (m, 1H), 7.77-7.73 (m, 1H), 7.46 (t, J = 7.7 Hz, 1H), |
| | | 6.32 (s, 1H), 2.65-2.55 (m, 2H), 2.16 (s, 3H), 2.02 (s, 3H), 1.89-1.83 |
| | | (m, 1H), 1.83-1.72 (m, 1H). |
| 5.62 | 611.2 | 1H NMR (400 MHz, DMSO-d6) δ 8.75 (s, 1H), 8.71 (d, J = 6.9 Hz, |
| | | 0.31H), 8.65 (d, J = 7.3 Hz, 0.69H), 8.56 (d, J = 1.9 Hz, 1H), 8.49 (d, J = |
| | | 2.3 Hz, 1H), 7.86 (t, J = 2.1 Hz, 1H), 7.80-7.73 (m, 1H), 7.73-7.61 |
| | | (m, 1H), 7.44 (td, J = 7.7, 1.8 Hz, 1H), 6.32 (s, 1H), 4.45-4.34 (m, |
| | | 0.37H), 4.34-4.22 (m, 0.34H), 3.95-3.74 (m, 0.65H), 2.67-2.55 (m, |
| | | 2H), 2.54-2.45 (m, 2H), 2.28-2.19 (m, 0.63H), 2.16 (s, 3H), 2.01 (s, |
| | | 3H), 1.91-1.81 (m, 2H), 1.82-1.74 (m, 1H). |
| 5.63 | 613.2 | 1H NMR (400 MHz, DMSO-d6) δ 8.82 (d, J = 7.0 Hz, 1H), 8.75 (s, |
| | | 1H), 8.56 (d, J = 2.0 Hz, 1H), 8.49 (d, J = 2.3 Hz, 1H), 7.86 (t, J = 2.2 |
| | | Hz, 1H), 7.82-7.75 (m, 1H), 7.75-7.65 (m, 1H), 7.46 (t, J = 7.7 Hz, |
| | | 1H), 6.32 (s, 1H), 5.33 (d, J = 3.7 Hz, 0.27H), 5.25-5.14 (m, 0.35H), |
| | | 4.68-4.33 (m, 0.65H), 2.65-2.57 (m, 2.7H), 2.49 (s, 3H), 2.48-2.35 |
| | | (m, 1H), 2.16 (s, 3H), 2.01 (s, 3H), 1.90-1.83 (m, 1H), 1.83-1.74 (m, 1H). |
| 6.1 | 520.0 | 1H NMR (400 MHz, DMSO-d6) δ 12.14 (s, 1H), 8.79 (s, 1H), 7.77 (t, J = |
| | | 8.3, 7.1 Hz, 1H), 7.71 (s, 1H), 7.38-7.26 (m, 2H), 7.20-7.05 (m, |
| | | 3H), 6.20 (s, 1H), 2.57-2.51 (m, 1H), 2.48-2.42 (m, 1H), 2.05 (s, |
| | | 3H), 1.94 (s, 3H), 1.68 (t, J = 7.4 Hz, 2H). |
| 6.2 | 478.1 | 1H NMR (400 MHz, DMSO-d6) δ 8.77 (s, 1H), 7.64 (s, 1H), 7.32 (dd, |
| | | J = 8.7, 5.5 Hz, 2H), 7.20-7.12 (m, 2H), 7.06 (d, J = 7.2 Hz, 1H), 7.04- |
| | | 6.98 (m, 1H), 6.87 (t, J = 8.4 Hz, 1H), 6.19 (d, J = 1.0 Hz, 1H), 2.55- |
| | | 2.46 (m, 2H), 2.04 (s, 3H), 1.90 (s, 3H), 1.67 (t, J = 7.4 Hz, 2H). |
| 6.3 | 520.0 | 1H NMR (400 MHz, DMSO-d6) δ 9.80 (s, 1H), 8.81 (d, J = 0.9 Hz, |
| | | 1H), 7.91 (s, 1H), 7.73-7.59 (m, 2H), 7.30 (tt, J = 9.4, 6.9 Hz, 3H), |
| | | 7.19-7.07 (m, 2H), 6.19 (s, 1H), 2.55-2.45(m, 2H), 2.10 (s, 3H), 2.06 |
| | | (s, 3H), 1.97-1.89 (m, 3H), 1.68 (m, 2H). |
| 6.4 | 556.2 | 1H NMR (400 MHz, DMSO-d6) δ 8.83 (s, 1H), 8.19 (d, J = 5.2 Hz, |
| | | 1H), 8.01 (td, J = 7.6, 2.1 Hz, 1H), 7.81-7.78 (m, 1H), 7.78-7.77 (m, |
| | | 1H), 7.75 (t, J = 59.3 Hz, 1H), 7.49-7.45 (m, 1H), 7.42 (t, J = 7.5 Hz, |
| | | 1H), 6.15 (dd, J = 2.1, 0.9 Hz, 1H), 3.02 (s, 3H), 2.89 (s, 3H), 2.46- |
| | | 2.28 (m, 2H), 2.07 (s, 3H), 1.93 (s, 3H), 1.70-1.56 (m, 2H). |
| 6.5 | 560.2 | 1H NMR (400 MHz, DMSO-d6) δ 9.84 (s, 1H), 8.77 (s, 1H), 8.20 (d, J = |
| | | 2.3 Hz, 1H), 8.03 (q, J = 2.1 Hz, 1H), 7.94 (d, J = 4.8 Hz, 1H), 7.85- |
| | | 7.78 (m, 2H), 7.76 (t, J = 59.3 Hz, 1H), 7.45 (t, J = 7.9 Hz, 1H), 7.32- |
| | | 7.27 (m, 1H), 6.16 (d, J = 2.7 Hz, 1H), 3.03 (s, 3H), 2.48-2.37 (m, |
| | | 1H), 2.36-2.28 (m, 1H), 2.04 (s, 3H), 1.93 (s, 3H), 1.71-1.54 (m, 2H). |
| 6.6 | 536.1 | 1H NMR (400 MHz, DMSO-d6) δ 8.73 (s, 1H), 7.90 (d, J = 3.4 Hz, |
| | | 1H), 7.71-7.67 (m, 1H), 7.61 (d, J = 7.8 Hz, 1H), 7.36-7.26 (m, 3H), |
| | | 7.20-7.07 (m, 2H), 7.00 (d, J = 7.8 Hz, 1H), 6.23-6.16 (m, 1H), 3.23 |
| | | (s, 6H), 2.49-2.42 (m, 2H), 2.02 (s, 3H), 1.93 (s, 3H), 1.77-1.59 (m, 2H). |
| 6.7 | 549.1 | 1H NMR (400 MHz, DMSO-d6) δ 8.84 (s, 1H), 7.99 (d, J = 5.2 Hz, |
| | | 1H), 7.86-7.76 (m, 1H), 7.34-7.27 (m, 2H), 7.23-7.08 (m, 3H), |
| | | 6.23-6.12 (m, 1H), 4.01-3.90 (m, 4H), 2.47-2.42 (m, 2H), 2.07 (s, |
| | | 3H), 1.92 (s, 3H), 1.76-1.60 (m, 2H), 1.45 (s, 3H). |
| 6.8 | 537.1 | 1H NMR (400 MHz, DMSO-d6) δ 8.87-8.75 (m, 2H), 8.29-8.15 (m, |
| | | 1H), 8.15-7.98 (m, 2H), 7.37-7.22 (m, 2H), 7.22-6.98 (m, 2H), |
| | | 6.26-6.13 (m, 1H), 3.33 (s, 6H), 2.48-2.39 (m, 2H), 2.05 (s, 3H), |
| | | 1.94 (s, 3H), 1.78-1.61 (m, 2H). |
| 6.9 | 525.3 | 1H NMR (400 MHz, DMSO-d6) δ 13.34 (s, 1H), 8.84 (s, 1H), 8.20 (d, |
| | | J = 5.5 Hz, 1H), 8.13 (d, J = 1.1 Hz, 1H), 8.09 (d, J = 6.3 Hz, 1H), 7.80 |
| | | (d, J = 7.0 Hz, 1H), 7.77-7.76 (m, 1H), 7.93-7.57 (m, 1H), 7.70 (d, J = |
| | | 11.5 Hz, 1H), 6.21-6.09 (m, 1H), 2.55-2.47 (m, 1H), 2.46-2.36 |
| | | (m, 1H), 2.08 (d, J = 1.6 Hz, 3H), 1.95 (s, 3H), 1.71-1.57 (m, 2H). |
| 6.10 | 528.2 | 1H NMR (400 MHz, DMSO-d6) δ 8.84 (s, 1H), 8.19 (d, J = 5.1 Hz, |
| | | 1H), 8.03 (td, J = 7.5, 1.8 Hz, 1H), 7.80 (d, J = 6.3 Hz, 2H), 7.74 (d, J = |
| | | 1.9 Hz, 1H), 7.73-7.66 (m, 2H), 7.68 (t, J = 60 Hz, 1H), 7.41 (t, J = |
| | | 7.7 Hz, 1H), 6.16 (s, 1H), 2.45-2.38 (m, 1H), 2.37-2.24 (m, 1H), |
| | | 2.08 (s, 3H), 1.94 (s, 3H), 1.72-1.56 (m, 2H). |
| 6.11 | 548.1 | 1H NMR (400 MHz, DMSO-d6) δ 10.39 (s, 1H), 8.81 (s, 1H), 7.71 (d, |
| | | J = 2.2 Hz, 1H), 7.40 (ddd, J = 8.3, 6.3, 1.9 Hz, 1H), 7.36-7.26 (m, |
| | | 3H), 7.15 (t, J = 8.9 Hz, 2H), 6.19 (s, 1H), 6.12-5.96 (m, 1H), 2.52- |
| | | 2.42 (m, 2H), 2.05 (s, 3H), 1.92 (s, 3H), 1.69 (m, 2H), 1.41 (m, 3H). |
| 6.12 | 524.2 | 1H NMR (400 MHz, DMSO-d6) δ 8.75 (s, 1H), 7.85 (s, 1H), 7.82- |
| | | 7.73 (m, 2H), 7.72-7.66 (m, 2H), 7.54-7.33 (m, 1H), 7.32 (s, 1H), |
| | | 7.16 (dt, J = 11.0, 5.4 Hz, 2H), 6.28 (s, 1H), 2.60-2.36 (m, 2H), 2.16 |
| | | (d, J = 2.0 Hz, 3H), 2.01 (s, 3H), 1.71 (m, 2H). |
| 6.13 | 546.1 | 1H NMR (400 MHz, DMSO-d6) δ 8.75 (s, 1H), 8.20 (d, J = 3.7 Hz, |
| | | 1H), 7.85 (s, 1H), 7.82-7.76 (m, 3H), 7.70 (s, 1H), 7.68 (t, J = 60 Hz, |
| | | 1H), 7.45 (t, J = 7.7 Hz, 1H), 6.24 (s, 1H), 2.47-2.35 (m, 2H), 2.16 (s, |
| | | 3H), 2.00 (s, 3H), 1.72-1.59 (m, 2H). |
| 6.14 | 574.1 | 1H NMR (400 MHz, DMSO-d6) δ 8.75 (s, 1H), 8.20 (d, J = 4.1 Hz, |
| | | 1H), 7.80 (d, J = 2.4 Hz, 1H), 7.78 (dd, J = 7.4, 1.8 Hz, 1H), 7.68 (t, J = |
| | | 60 Hz, 1H), 7.59-7.53 (m, 1H), 7.48 (t, J = 7.6 Hz, 1H), 6.23 (s, 1H), |
| | | 3.02 (s, 3H), 2.88-2.83 (m, 3H), 2.46-2.36 (m, 2H), 2.15 (s, 3H), |
| | | 1.99 (s, 3H), 1.75-1.60 (m, 2H). |
| 6.15 | 552.2 | 1H NMR (400 MHz, DMSO-d6) δ 8.75 (s, 1H), 8.11 (d, J = 7.8 Hz, |
| | | 1H), 7.77 (td, J = 7.3, 1.9 Hz, 1H), 7.61-7.53 (m, 1H), 7.50-7.43 (m, |
| | | 1H), 7.42-7.36 (m, 1H), 7.36-7.28 (m, 1H), 7.20-7.10 (m, 1H), |
| | | 6.27 (s, 1H), 3.02 (s, 3H), 2.86 (s, 3H), 2.58-2.39 (m, 2H), 2.16 (d, J = |
| | | 1.9 Hz, 3H), 2.00 (s, 3H), 1.76-1.63 (m, 2H). |
| 6.16 | 626.2 | 1H NMR (400 MHz, DMSO-d6) δ 10.71 (d, J = 7.9 Hz, 1H), 8.78 (s, |
| | | 1H), 8.22 (d, J = 11.9 Hz, 1H), 8.01 (d, J = 15.3 Hz, 1H), 7.85 (d, J = |
| | | 27.1 Hz, 1H), 7.32 (dd, J = 8.8, 5.4 Hz, 2H), 7.15 (t, J = 8.8 Hz, 2H), |
| | | 6.21 (s, 1H), 2.55-2.45 (m, 2H), 2.40 (p, J = 6.9 Hz, 1H), 2.06 (s, 3H), |
| | | 1.94 (s, 3H), 1.75-1.60 (m, 2H), 1.16 (d, J = 6.7 Hz, 3H), 0.56 (d, J = |
| | | 6.9 Hz, 3H). |
| 6.17 | 576.1 | 1H NMR (400 MHz, DMSO-d6) δ 10.40 (s, 1H), 8.81 (s, 1H), 7.73 (s, |
| | | 1H), 7.38 (d, J = 6.3 Hz, 1H), 7.32 (dd, J = 8.7, 5.5 Hz, 2H), 7.21 (d, J = |
| | | 10.6 Hz, 1H), 7.15 (t, J = 8.9 Hz, 2H), 6.19 (s, 1H), 2.55-2.45 (m, 2H), |
| | | 2.12 (p, J = 6.7 Hz, 1H), 2.05 (s, 3H), 1.93 (s, 3H), 1.68 (t, J = 7.4 Hz, |
| | | 2H), 0.99 (d, J = 6.8 Hz, 3H), 0.71 (d, J = 6.8 Hz, 3H). |
| 6.18 | 576.1 | 1H NMR (400 MHz, DMSO-d6) δ 10.39 (s, 1H), 8.81 (s, 1H), 7.74 (s, |
| | | 1H), 7.44 (d, J = 6.4 Hz, 1H), 7.32 (dd, J = 8.7, 5.5 Hz, 2H), 7.22 (d, J = |
| | | 10.7 Hz, 1H), 7.15 (t, J = 8.8 Hz, 2H), 6.19 (s, 1H), 2.55-2.45 (m, 2H), |
| | | 2.12 (p, J = 7.0 Hz, 1H), 2.05 (s, 3H), 1.93 (s, 3H), 1.68 (t, J = 7.5 Hz, |
| | | 2H), 0.99 (d, J = 6.8 Hz, 3H), 0.70 (d, J = 6.8 Hz, 3H). |
| 6.19 | 488.9 | 1H NMR (400 MHz, DMSO-d6) δ 8.94 (s, 1H), 8.75 (d, J = 4.9 Hz, |
| | | 1H), 8.38 (dd, J = 6.5, 4.9 Hz, 1H), 8.03-7.95 (m, 1H), 7.38-7.26 (m, |
| | | 2H), 7.16 (td, J = 8.9, 1.2 Hz, 2H), 6.22 (s, 1H), 2.49-2.44 (m, 2H), |
| | | 2.12 (s, 3H), 1.94 (s, 3H), 1.75-1.65 (m, 2H). |
| 6.20 | 552.2 | 1H NMR (400 MHz, DMSO-d6) δ 8.75 (s, 1H), 8.11 (d, J = 7.8 Hz, |
| | | 1H), 7.77 (td, J = 7.3, 1.9 Hz, 1H), 7.60-7.52 (m, 1H), 7.52-7.45 (m, |
| | | 1H), 7.41-7.36 (m, 1H), 7.36-7.28 (m, 1H), 7.20-7.09 (m, 1H), |
| | | 6.27 (s, 1H), 3.02 (s, 3H), 2.87 (d, J = 0.9 Hz, 3H), 2.60-2.40 (m, 2H), |
| | | 2.16 (s, 3H), 2.00 (s, 3H), 1.74-1.63 (m, 2H). |
| 6.21 | 546.1 | 1H NMR (400 MHz, DMSO-d6) δ 10.76 (s, 1H), 8.76 (s, 1H), 7.87 (t, J = |
| | | 8.0 Hz, 1H), 7.65 (d, J = 1.6 Hz, 1H), 7.38-7.27 (m, 2H), 7.15 (t, J = |
| | | 8.9 Hz, 2H), 6.86 (d, J = 8.1 Hz, 1H), 6.19 (s, 1H), 2.54 (d, J = 1.3 Hz, |
| | | 1H), 2.48-2.42 (m, 1H), 2.04 (s, 3H), 1.93 (s, 3H), 1.73-1.63 (m, |
| | | 2H), 1.38 (s, 6H). |
| 6.22 | 533.9 | 1H NMR (400 MHz, DMSO-d6) δ 8.84 (s, 1H), 8.05 (t, J = 7.9 Hz, |
| | | 1H), 7.78 (d, J = 1.6 Hz, 1H), 7.45-7.36 (m, 2H), 7.36-7.27 (m, 2H), |
| | | 7.19-7.08 (m, 2H), 6.20 (s, 1H), 3.01 (s, 3H), 2.96 (s, 3H), 2.58-2.43 |
| | | (m, 2H), 2.07 (s, 3H), 1.94 (s, 3H), 1.68 (t, J = 7.4 Hz, 2H). |
| 6.23 | 505.9 | 1H NMR (400 MHz, DMSO-d6) δ 8.85 (s, 1H), 8.15 (s, 1H), 8.08 (t, J = |
| | | 8.0 Hz, 1H), 7.91-7.78 (m, 3H), 7.60 (s, 1H), 7.37-7.28 (m, 2H), |
| | | 7.21-7.09 (m, 2H), 6.21 (s, 1H), 2.60-2.54 (m, 1H), 2.49-2.43 (m, |
| | | 1H), 2.08 (s, 3H), 1.95 (s, 3H), 1.73-1.64 (m, 2H). |
| 6.24 | 530.1 | 1H NMR (400 MHz, DMSO-d6) δ 10.40 (d, J = 3.1 Hz, 1H), 8.73 (s, |
| | | 1H), 7.97 (d, J = 2.0 Hz, 1H), 7.72 (td, J = 7.5, 1.6 Hz, 1H), 7.60 (dd, J = |
| | | 14.2, 1.5 Hz, 1H), 7.38 (d, J = 7.7 Hz, 1H), 7.35-7.25 (m, 2H), 7.18- |
| | | 7.07 (m, 2H), 6.20 (s, 1H), 2.60-2.39 (m, 2H), 2.03 (s, 3H), 1.93 (s, |
| | | 3H), 1.68 (ddd, J = 9.5, 6.1, 2.9 Hz, 2H), 1.40 (d, J = 2.2 Hz, 3H). |
| 6.25 | 540.8 | 1H NMR (400 MHz, DMSO-d6) δ 8.87 (s, 1H), 8.32 (td, J = 7.5, 1.8 |
| | | Hz, 1H), 8.01-7.92 (m, 1H), 7.89 (d, J = 1.9 Hz, 1H), 7.62 (t, J = 7.8 |
| | | Hz, 1H), 7.38-7.28 (m, 2H), 7.21-7.09 (m, 2H), 6.22 (s, 1H), 3.40 (s, |
| | | 3H), 2.50-2.40 (m, 2H), 2.10 (s, 3H), 1.96 (s, 3H), 1.75-1.62 (m, 2H). |
| 6.26 | 536.0 | 1H NMR (400 MHz, DMSO-d6) δ 11.38 (s, 1H), 8.80 (s, 1H), 8.11 (s, |
| | | 1H), 7.91 (dd, J = 7.9, 1.4 Hz, 1H), 7.82-7.65 (m, 2H), 7.39-7.26 (m, |
| | | 2H), 7.20-7.05 (m, 2H), 6.21 (s, 1H), 2.55-2.45(m, 2H), 2.06 (s, 3H), |
| | | 1.94 (s, 3H), 1.75-1.59 (m, 2H). |
| 6.27 | 541.8 | 1H NMR (400 MHz, Methanol-d4) δ 8.86 (s, 1H), 8.48 (s, 1H), 8.42 (d, |
| | | J = 2.6 Hz, 1H), 8.37-8.28 (m, 1H), 8.11-8.02 (m, 1H), 7.82 (d, J = |
| | | 2.0 Hz, 1H), 7.72-7.63 (m, 1H), 7.60 (t, J = 7.8 Hz, 1H), 6.30 (s, 1H), |
| | | 3.36-3.31 (s, 3H), 2.82-2.72 (m, 1H), 2.70-2.58 (m, 1H), 2.19 (s, |
| | | 3H), 2.08 (s, 3H), 1.91-1.76 (m, 2H). |
| 6.28 | 557.8 | 1H NMR (400 MHz, Methanol-d4) δ 8.86 (s, 1H), 8.56 (dd, J = 8.1, 2.1 |
| | | Hz, 2H), 8.32 (td, J = 7.5, 1.8 Hz, 1H), 8.10-8.02 (m, 1H), 7.98 (t, J = |
| | | 2.1 Hz, 1H), 7.83 (d, J = 2.0 Hz, 1H), 7.59 (t, J = 7.8 Hz, 1H), 6.29 (s, |
| | | 1H), 3.36 (s, 3H), 2.83-2.72 (m, 1H), 2.67-2.55 (m, 1H), 2.19 (s, |
| | | 3H), 2.08 (s, 3H), 1.85 (t, J = 7.5 Hz, 2H). |
| 6.29 | 506.8 | 1H NMR (400 MHz, Methanol-d4) δ 8.83 (s, 1H), 8.59-8.41 (m, 2H), |
| | | 8.07 (td, J = 7.5, 1.9 Hz, 1H), 7.94-7.83 (m, 1H), 7.83-7.70 (m, 2H), |
| | | 7.45 (t, J = 7.7 Hz, 1H), 6.30 (s, 1H), 2.83-2.73 (m, 1H), 2.71-2.60 |
| | | (m, 1H), 2.18 (s, 3H), 2.08 (s, 3H), 1.91-1.78 (m, 2H). |
| 6.30 | 546.1 | 1H NMR (400 MHz, DMSO-d6) δ 10.04 (s, 1H), 8.81 (s, 1H), 7.92 (t, J = |
| | | 7.5 Hz, 1H), 7.69 (s, 1H), 7.64 (t, J = 7.2 Hz, 1H), 7.38-7.22 (m, |
| | | 3H), 7.15 (t, J = 8.9 Hz, 2H), 6.19 (s, 1H), 2.55-2.45 (m, 2H), 2.06 (s, |
| | | 3H), 2.02-1.98 (m, 1H), 1.94 (s, 3H), 1.68 (t, J = 7.4 Hz, 2H), 0.81 (d, |
| | | J = 5.0 Hz, 4H). |
| 6.31 | 564.1 | 1H NMR (400 MHz, DMSO-d6) δ 9.41 (s, 1H), 8.82 (s, 1H), 8.14- |
| | | 8.02 (m, 1H), 7.74 (d, J = 1.7 Hz, 1H), 7.69 (td, J = 7.6, 1.7 Hz, 1H), |
| | | 7.37-7.26 (m, 3H), 7.15 (t, J = 8.9 Hz, 2H), 6.19 (s, 1H), 6.02 (s, 1H), |
| | | 2.55-2.43 (m, 2H), 2.06 (s, 3H), 1.94 (s, 3H), 1.67 (t, J = 7.5 Hz, 2H), |
| | | 1.38 (s, 3H), 1.37 (s, 3H). |
| 6.32 | 581.1 | 1H NMR (400 MHz, DMSO-d6) δ 9.41 (t, J = 1.5 Hz, 1H), 8.87-8.64 |
| | | (m, 1H), 8.55 (d, J = 1.9 Hz, 1H), 8.48 (d, J = 2.3 Hz, 1H), 8.15-7.98 |
| | | (m, 1H), 7.85 (dt, J = 7.2, 2.1 Hz, 1H), 7.75 (d, J = 1.6 Hz, 1H), 7.70 |
| | | (td, J = 7.6, 1.7 Hz, 1H), 7.32 (t, J = 8.0 Hz, 1H), 6.23 (s, 1H), 6.02 (d, J = |
| | | 2.1 Hz, 1H), 2.64-2.54 (m, 2H), 2.07 (d, J = 2.5 Hz, 3H), 2.00- |
| | | 1.91 (m, 3H), 1.83 (dt, J = 8.6, 5.9 Hz, 1H), 1.75 (dt, J = 8.9, 5.7 Hz, |
| | | 1H), 1.38 (d, J = 2.0 Hz, 6H). |
| 6.33 | 559.8 | 1H NMR (400 MHz, DMSO-d6) δ 8.79 (s, 1H), 8.49 (t, J = 1.8 Hz, |
| | | 1H), 8.44 (d, J = 2.7 Hz, 1H), 8.15-8.00 (m, 2H), 7.72-7.62 (m, 2H), |
| | | 6.33 (s, 1H), 3.39 (s, 3H), 2.66-2.58 (m, 2H), 2.18 (s, 3H), 2.03 (s, |
| | | 3H), 1.90-1.77(m, 2H). |
| 6.34 | 538.8 | 1H NMR (400 MHz, DMSO-d6) δ 8.75 (s, 1H), 8.53-8.46 (m, 1H), |
| | | 8.44 (d, J = 2.7 Hz, 1H), 8.41-8.32 (m, 1H), 7.82-7.71 (m, 2H), 7.71- |
| | | 7.63 (m, 1H), 7.46 (t, J = 7.7 Hz, 1H), 6.32 (s, 1H), 2.79 (d, J = 4.6 |
| | | Hz, 3H), 2.66-2.58 (m, 2H), 2.16 (s, 3H), 2.02 (s, 3H), 1.88-1.77 (m, 2H). |
| 6.35 | 564.9 | 1H NMR (400 MHz, DMSO-d6) δ 8.74 (d, J = 0.8 Hz, 1H), 8.53-8.47 |
| | | (m, 2H), 8.44 (d, J = 2.6 Hz, 1H), 7.80-7.72 (m, 1H), 7.72-7.63 (m, |
| | | 2H), 7.44 (t, J = 7.7 Hz, 1H), 6.32 (s, 1H), 2.90-2.80 (m, 1H), 2.66- |
| | | 2.58 (m, 2H), 2.16 (s, 3H), 2.01 (s, 3H), 1.88-1.77 (m, 2H), 0.74- |
| | | 0.66 (m, 2H), 0.58-0.50 (m, 2H). |
| 6.36 | 575.8 | |
| 6.37 | 524.9 | 1H NMR (400 MHz, DMSO-d6) δ 8.75 (s, 1H), 8.50 (t, J = 1.8 Hz, |
| | | 1H), 8.44 (d, J = 2.7 Hz, 1H), 7.85 (s, 1H), 7.82-7.74 (m, 2H), 7.74- |
| | | 7.64 (m, 2H), 7.45 (t, J = 7.7 Hz, 1H), 6.32 (s, 1H), 2.67-2.58 (m, |
| | | 2H), 2.16 (s, 3H), 2.02 (s, 3H), 1.88-1.76 (m, 2H). |
| 6.38 | 540.9 | 1H NMR (400 MHz, DMSO-d6) δ 8.75 (d, J = 0.9 Hz, 1H), 8.56 (d, J = |
| | | 1.9 Hz, 1H), 8.49 (d, J = 2.3 Hz, 1H), 7.93-7.82 (m, 2H), 7.82-7.74 |
| | | (m, 2H), 7.71 (s, 1H), 7.45 (t, J = 7.7 Hz, 1H), 6.32 (s, 1H), 2.66-2.55 |
| | | (m, 2H), 2.16 (s, 3H), 2.02 (s, 3H), 1.90-1.74 (m, 2H). |
| 6.39 | 575.0 | 1H NMR (400 MHz, DMSO-d6) δ 8.77 (s, 1H), 8.55 (d, J = 2.0 Hz, |
| | | 1H), 8.48 (d, J = 2.3 Hz, 1H), 8.12-7.92 (m, 2H), 7.86 (t, J = 2.2 Hz, |
| | | 1H), 7.61 (t, J = 7.8 Hz, 1H), 6.32 (s, 1H), 3.27 (s, 3H), 2.68-2.55 (m, |
| | | 2H), 2.21-2.09 (m, 3H), 2.07-1.94 (m, 3H), 1.89-1.71 (m, 2H). |
| 6.40 | 576.8 | 1H NMR (400 MHz, Methanol-d4) δ 9.11 (s, 1H), 8.84 (s, 1H), 8.51 |
| | | (dd, J = 12.7, 2.1 Hz, 2H), 8.02 (td, J = 7.6, 1.9 Hz, 1H), 7.89 (s, 1H), |
| | | 7.77 (s, 1H), 7.59 (td, J = 7.2, 1.8 Hz, 1H), 7.48 (t, J = 7.7 Hz, 1H), 2.75 |
| | | (td, J = 7.5, 4.7 Hz, 1H), 2.60 (td, J = 7.6, 4.7 Hz, 1H), 2.45 (s, 3H), |
| | | 2.19 (s, 3H), 2.07 (s, 3H), 1.82 (t, J = 7.6 Hz, 2H). |
| 6.41 | 576.8 | 1H NMR (400 MHz, Methanol-d4) δ 8.90 (s, 1H), 8.85 (s, 1H), 8.56 (d, |
| | | J = 7.2 Hz, 2H), 7.97 (m, 2H), 7.79 (dd, J = 11.7, 1.8 Hz, 1H), 7.54 (td, |
| | | J = 7.3, 1.9 Hz, 1H), 7.46 (t, J = 7.7 Hz, 1H), 6.28 (s, 1H), 2.76 (td, J = |
| | | 7.9, 4.6 Hz, 1H), 2.61 (td, J = 7.6, 4.6 Hz, 1H), 2.45 (s, 3H), 2.19 (s, |
| | | 3H), 2.07 (s, 3H), 1.83 (t, J = 7.6 Hz, 2H). |
| 6.42 | 561.8 | 1H NMR (400 MHz, Methanol-d4) δ 8.85 (s, 1H), 8.49 (d, J = 1.9 Hz, |
| | | 1H), 8.45 (d, J = 2.3 Hz, 1H), 8.21 (td, J = 7.6, 1.8 Hz, 1H), 8.10 (s, |
| | | 1H), 7.80 (m, 2H), 7.72 (td, J = 7.2, 6.5, 1.9 Hz, 1H), 7.57 (t, J = 7.7 |
| | | Hz, 1H), 6.28 (s, 1H), 3.92 (s, 3H), 2.74 (m, 1H), 2.62-2.53 (m, 1H), |
| | | 2.19 (s, 3H), 2.07 (s, 3H), 1.81 (t, J = 7.6 Hz, 2H) |
| 6.43 | 565.0 | 1H NMR (400 MHz, DMSO-d6) δ 8.78 (s, 1H), 8.00 (t, J = 7.2 Hz, |
| | | 1H), 7.89 (t, J = 7.5 Hz, 1H), 7.72-7.62 (m, 3H), 6.26 (s, 1H), 2.63- |
| | | 2.55 (m, 2H), 2.46 (s, 3H), 2.30 (d, J = 1.2 Hz, 3H), 2.16 (s, 3H), 1.98 |
| | | (s, 3H), 1.72-1.64 (m, 2H). |
| 6.44 | 566.0 | 1H NMR (400 MHz, DMSO-d6) δ 8.75 (d, J = 0.8 Hz, 1H), 7.79-7.66 |
| | | (m, 3H), 7.50 (t, J = 7.7 Hz, 1H), 6.30-6.23 (m, 1H), 4.15 (s, 3H), |
| | | 2.57 (tdd, J = 11.0, 5.7, 2.5 Hz, 2H), 2.24 (d, J = 1.9 Hz, 3H), 2.15 (s, |
| | | 3H), 1.98 (s, 3H), 1.73-1.64 (m, 2H). |
| 6.45 | 551.0 | 1H NMR (400 MHz, DMSO-d6) δ 8.79 (s, 1H), 8.06-7.88 (m, 3H), |
| | | 7.80-7.63 (m, 3H), 6.26 (s, 1H), 2.63-2.55 (m, 2H), 2.17 (s, 3H), |
| | | 1.98 (s, 3H), 1.71-1.64 (m, 2H). |
| 6.46 | 551.8 | 1H NMR (400 MHz, Methanol-d4) δ 9.00 (s, 1H), 8.73 (s, 1H), 8.01 |
| | | (ddd, J = 8.1, 6.5, 1.7 Hz, 1H), 7.81 (td, J = 7.6, 7.1, 1.7 Hz, 1H), 7.66 |
| | | (t, J = 7.9 Hz, 1H), 7.58 (s, 1H), 6.24 (s, 1H), 2.76 (ddd, J = 8.7, 6.0, 4.4 |
| | | Hz, 1H), 2.57 (ddd, J = 9.0, 6.1, 4.4 Hz, 1H), 2.52 (s, 3H), 2.24 (s, 3H), |
| | | 2.08 (s, 3H), 1.77 (ddd, J = 8.8, 6.2, 5.1 Hz, 1H), 1.70 (dt, J = 9.1, 5.5 |
| | | Hz, 1H). |
| 6.47 | 562.8 | 1H NMR (400 MHz, Methanol-d4) δ 9.00 (s, 1H), 8.74 (d, J = 0.8 Hz, |
| | | 1H), 8.49 (s, 1H), 8.47-8.40 (m, 1H), 8.01 (ddd, J = 8.1, 6.5, 1.8 Hz, |
| | | 1H), 7.82 (ddd, J = 8.0, 7.0, 1.8 Hz, 1H), 7.73-7.61 (m, 2H), 6.32 (d, J = |
| | | 1.0 Hz, 1H), 2.77 (ddd, J = 8.3, 6.5, 4.6 Hz, 1H), 2.66 (q, J = 6.8, 6.1 |
| | | Hz, 1H), 2.52 (s, 3H), 2.26-2.23 (s, 3H), 2.10 (s, 3H), 1.85 (ddd, J = |
| | | 8.5, 6.5, 2.1 Hz, 2H). |
| 6.48 | 598.8 | 1H NMR (400 MHz, Methanol-d4) δ 8.72 (s, 1H), 8.01 (s, 1H), 7.96 |
| | | (ddd, J = 8.0, 6.5, 1.8 Hz, 1H), 7.77 (ddd, J = 8.6, 7.1, 1.8 Hz, 1H), 7.71 |
| | | (s, 1H), 7.62 (td, J = 7.9, 0.8 Hz, 1H), 7.43 (s, 1H), 6.22 (s, 1H), 2.58 |
| | | (ddd, J = 8.6, 5.9, 4.5 Hz, 1H), 2.47 (d, J = 1.2 Hz, 3H), 2.45 (s, 3H), |
| | | 2.43-2.36 (m, 1H), 2.24 (s, 3H), 2.08 (s, 3H), 1.71-1.59 (m, 2H). |
| 6.49 | 559.0 | 1H NMR (400 MHz, DMSO-d6) δ 8.76 (s, 1H), 8.39 (dt, J = 4.7, 1.5 |
| | | Hz, 1H), 8.10-7.95 (m, 2H), 7.71 (ddd, J = 10.0, 8.3, 1.4 Hz, 1H), |
| | | 7.61 (t, J = 7.8 Hz, 1H), 7.34 (dt, J = 8.6, 4.4 Hz, 1H), 6.40 (s, 1H), 3.26 |
| | | (s, 3H), 2.97-2.84 (m, 2H), 2.17 (s, 3H), 2.03 (s, 3H), 1.84 (ddd, J = |
| | | 8.6, 5.9, 4.2 Hz, 1H), 1.77 (ddd, J = 8.5, 6.1, 4.2 Hz, 1H). |
| 6.50 | 559.0 | 1H NMR (400 MHz, DMSO-d6) δ 8.76 (s, 1H), 8.42-8.37 (m, 1H), |
| | | 8.09-7.95 (m, 2H), 7.71 (dd, J = 10.1, 8.4 Hz, 1H), 7.60 (t, J = 7.8 Hz, |
| | | 1H), 7.33 (dt, J = 8.6, 4.4 Hz, 1H), 6.39 (s, 1H), 3.25 (s, 3H), 2.91 (qd, J = |
| | | 7.6, 6.9, 4.3 Hz, 2H), 2.17 (s, 3H), 2.02 (s, 3H), 1.84 (ddd, J = 9.4, |
| | | 5.9, 4.2 Hz, 1H), 1.77 (ddd, J = 8.7, 6.0, 4.0 Hz, 1H). |
| 6.51 | 580.0 | 1H NMR (400 MHz, DMSO-d6) δ 8.77 (s, 1H), 8.19 (s, 1H), 8.11- |
| | | 7.96 (m, 2H), 7.92-7.73 (m, 2H), 7.67-7.58 (m, 1H), 6.23 (s, 1H), |
| | | 3.31 (s, 3H), 2.45-2.35 (m, 2H), 2.16 (s, 3H), 2.00 (s, 3H), 1.64 (t, J = |
| | | 7.4 Hz, 2H). |
| 6.52 | 580.0 | 1H NMR (400 MHz, DMSO-d6) δ 8.77 (s, 1H), 8.20 (s, 1H), 8.10- |
| | | 7.96 (m, 2H), 7.93-7.71 (m, 2H), 7.65-7.58 (m, 1H), 6.23 (s, 1H), |
| | | 3.28 (s, 3H), 2.46-2.36 (m, 2H), 2.16 (s, 3H), 2.00 (s, 3H), 1.64 (dd, J = |
| | | 8.3, 6.5 Hz, 2H). |
| 6.53 | 560.8 | 1H NMR (400 MHz, Methanol-d4) δ 9.09 (d, J = 5.2 Hz, 1H), 8.77 (s, |
| | | 1H), 8.31 (d, J = 5.2 Hz, 1H), 7.30 (dd, J = 8.7, 5.4 Hz, 2H), 7.07 (t, J = |
| | | 8.8 Hz, 2H), 6.28 (s, 1H), 2.91 (s, 3H), 2.64 (dt, J = 8.2, 5.8 Hz, 1H), |
| | | 2.58-2.51 (m, 1H), 2.44 (s, 3H), 2.27 (s, 3H), 2.12 (s, 3H), 1.73 (ddd, |
| | | J = 8.3, 6.4, 2.0 Hz, 2H). |
| 6.54 | 576.8 | 1H NMR (400 MHz, Methanol-d4) δ 8.78 (s, 1H), 8.70-8.51 (m, 1H), |
| | | 7.99 (t, J = 4.9 Hz, 1H), 7.36-7.18 (m, 3H), 7.16-7.02 (m, 2H), 6.33- |
| | | 6.08 (m, 1H), 2.66-2.58 (m, 4H), 2.58-2.48 (m, 1H), 2.44 (s, 3H), |
| | | 2.27 (s, 3H), 2.10 (s, 3H), 1.78-1.58 (m, 2H). |
| 6.55 | 560.7 | 1H NMR (400 MHz, Methanol-d4) δ 9.09 (d, J = 5.3 Hz, 1H), 8.78 (s, |
| | | 1H), 8.44 (s, 1H), 8.36 (d, J = 2.7 Hz, 1H), 8.30 (d, J = 5.2 Hz, 1H), |
| | | 7.61-7.53 (m, 1H), 6.34 (s, 1H), 2.90 (s, 3H), 2.81-2.72 (m, 1H), |
| | | 2.66 (m, 1H), 2.43 (s, 3H), 2.27 (s, 3H), 2.13 (s, 3H), 1.89-1.80 (m, 2H). |
| 6.56 | 558.0 | 1H NMR (400 MHz, DMSO-d6) δ 8.76 (s, 1H), 8.09-7.94 (m, 2H), |
| | | 7.60 (t, J = 7.8 Hz, 1H), 7.36-7.27 (m, 2H), 7.21-7.10 (m, 2H), 6.28 |
| | | (s, 1H), 3.24 (s, 3H), 2.58-2.52 (m, 1H), 2.48-2.42 (m, 1H), 2.17 (s, |
| | | 3H), 2.01 (s, 3H), 1.70 (dd, J = 8.3, 6.6 Hz, 2H). |
| 6.57 | 558.0 | 1H NMR (400 MHz, DMSO-d6) δ 8.77 (s, 1H), 8.09-7.94 (m, 2H), |
| | | 7.60 (t, J = 7.8 Hz, 1H), 7.37-7.27 (m, 2H), 7.22-7.09 (m, 2H), 6.27 |
| | | (s, 1H), 3.26 (s, 3H), 2.59-2.52 (m, 1H), 2.48-2.43 (m, 1H), 2.17 (s, |
| | | 3H), 2.01 (s, 3H), 1.70 (dd, J = 8.3, 6.6 Hz, 2H). |
| 6.58 | 602.1 | 1H NMR (400 MHz, DMSO-d6) δ 8.70 (t, J = 0.8 Hz, 1H), 8.49 (s, |
| | | 1H), 8.43 (d, J = 2.7 Hz, 1H), 7.67 (d, J = 10.4 Hz, 1H), 7.37 (dt, J = |
| | | 8.1, 3.8 Hz, 1H), 7.21-7.09 (m, 2H), 6.31 (s, 1H), 3.10 (d, J = 0.8 Hz, |
| | | 3H), 2.79 (s, 3H), 2.79 (s, 3H), 2.61 (t, J = 7.5 Hz, 2H), 2.13 (s, 3H), |
| | | 1.99 (s, 3H), 1.87-1.75 (m, 2H). |
| 6.59 | 615.1 | 1H NMR (400 MHz, DMSO-d6) δ 8.71 (d, J = 0.8 Hz, 1H), 8.49 (t, J = |
| | | 1.9 Hz, 1H), 8.43 (d, J = 2.7 Hz, 1H), 7.67 (dt, J = 10.3, 2.3 Hz, 1H), |
| | | 7.36-7.26 (m, 1H), 7.25-7.10 (m, 2H), 6.31 (s, 1H), 4.09 (m, 2H), |
| | | 3.96-3.82 (m, 2H), 3.57-3.28 (m, 4H), 2.62 (t, J = 7.3 Hz, 2H), 2.16- |
| | | 2.12 (m, 3H), 2.00 (s, 3H), 1.82 (m, 2H). |
| 6.60 | 599.0 | 1H NMR (400 MHz, DMSO-d6) δ 8.70 (d, J = 0.8 Hz, 1H), 8.49 (t, J = |
| | | 1.8 Hz, 1H), 8.43 (d, J = 2.7 Hz, 1H), 7.66 (dt, J = 10.4, 2.2 Hz, 1H), |
| | | 7.30-7.16 (m, 2H), 7.16-7.09 (m, 1H), 6.31 (s, 1H), 3.49-3.17 (m, |
| | | 4H), 2.61 (t, J = 7.4 Hz, 2H), 2.28-2.04 (m, 7H), 2.00 (s, 3H), 1.80 |
| | | (dd, J = 11.9, 5.3 Hz, 2H). |
| 6.61 | 563.7 | 1H NMR (400 MHz, Methanol-d4) δ 8.72 (s, 1H), 8.36 (d, J = 4.7 Hz, |
| | | 1H), 8.16 (s, 1H), 7.97 (td, J = 7.5, 6.7, 1.8 Hz, 1H), 7.81 (td, J = 7.3, |
| | | 6.7, 1.8 Hz, 1H), 7.65-7.54 (m, 2H), 7.39 (m, 1H), 6.36-6.31 (s, 1H), |
| | | 3.92 (d, J = 1.7 Hz, 3H), 3.04 (ddd, J = 8.9, 6.1, 4.4 Hz, 1H), 2.90 |
| | | (dddd, J = 8.9, 6.0, 4.4, 1.4 Hz, 1H), 2.25 (s, 3H), 2.11 (s, 3H), 2.01 |
| | | (ddd, J = 8.8, 5.9, 4.6 Hz, 1H), 1.78 (ddd, J = 8.8, 6.1, 4.6 Hz, 1H). |
| 6.62 | 577.8 | 1H NMR (400 MHz, Methanol-d4) δ 8.78 (s, 1H), 8.63 (d, J = 4.9 Hz, |
| | | 1H), 8.36 (d, J = 4.8 Hz, 1H), 8.00 (t, J = 4.8 Hz, 1H), 7.57 (ddd, J = |
| | | 9.8, 8.3, 1.4 Hz, 1H), 7.30 (dt, J = 8.6, 4.4 Hz, 1H), 6.35 (s, 1H), 3.04 |
| | | (ddd, J = 8.8, 6.1, 4.3 Hz, 1H), 2.90 (ddt, J = 5.8, 4.4, 3.1 Hz, 1H), 2.60 |
| | | (s, 3H), 2.45 (s, 3H), 2.27 (s, 3H), 2.11 (s, 3H), 2.07-1.93 (m, 1H), |
| | | 1.79 (ddd, J = 8.8, 6.1, 4.6 Hz, 1H). |
| 6.63 | 598.8 | 1H NMR (400 MHz, Methanol-d4) δ 8.78 (d, J = 0.8 Hz, 1H), 8.63 (d, J = |
| | | 4.9 Hz, 1H), 8.04-7.93 (m, 2H), 7.72 (s, 1H), 7.44 (t, J = 59.8 Hz, |
| | | 1H), 6.23 (d, J = 1.0 Hz, 1H), 2.64-2.54 (m, 4H), 2.44 (s, 4H), 2.29- |
| | | 2.23 (m, 3H), 2.09 (s, 3H), 1.75-1.57 (m, 2H) |
| 6.64 | 562.8 | 1H NMR (400 MHz, Methanol-d4) δ 9.32 (s, 1H), 8.74 (s, 1H), 8.44 (s, |
| | | 1H), 8.37 (d, J = 2.6 Hz, 1H), 7.97 (ddd, J = 8.1, 6.4, 1.7 Hz, 1H), 7.90 |
| | | (ddd, J = 8.6, 7.1, 1.7 Hz, 1H), 7.75 (d, J = 1.4 Hz, 1H), 7.65 (td, J = |
| | | 8.0, 1.0 Hz, 1H), 7.58 (dt, J = 9.7, 2.3 Hz, 1H), 6.32 (d, J = 1.0 Hz, 1H), |
| | | 2.75 (ddd, J = 8.1, 6.8, 4.7 Hz, 1H), 2.68-2.60 (m, 1H), 2.47 (s, 3H), |
| | | 2.26 (s, 3H), 2.11 (s, 3H), 1.86-1.79 (m, 2H). |
| 6.65 | 578.8 | 1H NMR (400 MHz, Methanol-d4) δ 8.79 (s, 1H), 8.63 (d, J = 4.9 Hz, |
| | | 1H), 8.46 (s, 1H), 8.40 (d, J = 2.6 Hz, 1H), 7.98 (t, J = 4.9 Hz, 1H), 7.64 |
| | | (dt, J = 9.9, 2.2 Hz, 1H), 6.33 (s, 1H), 2.77 (ddd, J = 8.6, 6.3, 4.6 Hz, |
| | | 1H), 2.69-2.60 (m, 1H), 2.57 (s, 3H), 2.42 (s, 3H), 2.27 (s, 3H), 2.11 |
| | | (s, 3H), 1.85 (tt, J = 9.3, 5.7 Hz, 2H). |
| 6.66 | 563.7 | 1H NMR (400 MHz, Methanol-d4) δ 9.11 (s, 1H), 8.83-8.75 (m, 1H), |
| | | 8.57 (d, J = 4.9 Hz, 1H), 8.48 (s, 1H), 8.42 (d, J = 2.6 Hz, 1H), 7.87 (t, |
| | | J = 4.8 Hz, 1H), 7.73-7.62 (m, 1H), 6.34 (s, 1H), 2.77 (dt, J = 8.9, 5.8 |
| | | Hz, 1H), 2.65 (q, J = 6.6, 5.9 Hz, 1H), 2.50 (s, 3H), 2.31-2.24 (m, |
| | | 3H), 2.12 (d, J = 6.8 Hz, 3H), 1.85 (tt, J = 10.1, 5.9 Hz, 2H). |
| 6.67 | 588.7 | 1H NMR (400 MHz, Methanol-d4) δ 8.72 (s, 1H), 8.50 (s, 1H), 8.45 (d, |
| | | J = 2.6 Hz, 1H), 8.07 (s, 1H), 7.97 (td, J = 7.3, 6.8, 1.8 Hz, 1H), 7.81 |
| | | (ddd, J = 8.2, 6.7, 1.8 Hz, 1H), 7.72 (dt, J = 9.7, 2.3 Hz, 1H), 7.61 (t, J = |
| | | 7.7 Hz, 1H), 6.32 (d, J = 1.0 Hz, 1H), 3.75-3.64 (m, 1H), 2.77 (ddd, J = |
| | | 8.4, 6.5, 4.7 Hz, 1H), 2.66 (q, J = 6.6, 5.9 Hz, 1H), 2.25 (s, 3H), 2.11 |
| | | (s, 3H), 1.85 (ddd, J = 8.8, 6.3, 2.7 Hz, 2H), 1.09-0.99 (m, 4H). |
| 6.68 | 561.9 | 1H NMR (400 MHz, Methanol-d4) δ 8.85 (s, 1H), 8.51 (dd, J = 14.0, |
| | | 2.1 Hz, 2H), 8.19 (ddd, J = 7.9, 6.6, 1.8 Hz, 1H), 8.10 (td, J = 7.5, 1.9 |
| | | Hz, 1H), 7.88 (t, J = 2.1 Hz, 1H), 7.83 (d, J = 2.0 Hz, 1H), 7.53 (t, J = |
| | | 7.8 Hz, 1H), 6.28 (s, 1H), 4.48 (s, 3H), 2.82-2.70 (m, 1H), 2.60 (td, J = |
| | | 7.7, 4.7 Hz, 1H), 2.19 (s, 3H), 2.09 (s, 3H), 1.82 (t, J = 7.6 Hz, 2H). |
| 6.69 | 612.0 | 1H NMR (400 MHz, DMSO-d6) δ 8.80 (d, J = 0.8 Hz, 1H), 8.55 (d, J = |
| | | 1.9 Hz, 1H), 8.48 (d, J = 2.3 Hz, 1H), 8.31-8.27 (m, 1H), 8.18-8.12 |
| | | (m, 1H), 7.86 (t, J = 2.1 Hz, 1H), 7.78 (t, J = 7.9 Hz, 1H), 7.46 (t, J = |
| | | 51.9 Hz, 1H), 6.32 (s, 1H), 2.65-2.56 (m, 2H), 2.18 (s, 3H), 2.01 (s, |
| | | 3H), 1.88-1.75 (m, 2H). |
| 6.70 | 586.1 | 1H NMR (400 MHz, DMSO-d6) δ 8.79 (d, J = 0.8 Hz, 1H), 8.49 (t, J = |
| | | 1.9 Hz, 1H), 8.43 (d, J = 2.7 Hz, 1H), 8.12-8.05 (m, 1H), 8.01 (ddd, |
| | | J = 8.3, 6.7, 1.8 Hz, 1H), 7.69-7.61 (m, 2H), 6.32 (s, 1H), 3.08-2.99 |
| | | (m, 1H), 2.65-2.57 (m, 2H), 2.17 (s, 3H), 2.02 (s, 3H), 1.88-1.74 (m, |
| | | 2H), 1.19-1.08 (m, 4H). |
| 6.71 | 596.0 | 1H NMR (400 MHz, DMSO-d6) δ 8.80 (d, J = 0.8 Hz, 1H), 8.49 (t, J = |
| | | 1.9 Hz, 1H), 8.43 (d, J = 2.7 Hz, 1H), 8.32-8.26 (m, 1H), 8.18-8.12 |
| | | (m, 1H), 7.79 (t, J = 7.9 Hz, 1H), 7.66 (dt, J = 10.3, 2.3 Hz, 1H), 7.46 (t, |
| | | J = 51.9 Hz, 1H), 6.32 (s, 1H), 2.66-2.58 (m, 2H), 2.18 (s, 3H), 2.02 |
| | | (s, 3H), 1.86-1.76 (m, 2H). |
| 6.72 | 618.1 | 1H NMR (400 MHz, DMSO-d6) δ 8.78 (s, 1H), 8.49 (t, J = 1.8 Hz, |
| | | 1H), 8.43 (d, J = 2.7 Hz, 1H), 8.09-7.97 (m, 2H), 7.70-7.58 (m, 2H), |
| | | 6.32 (s, 1H), 3.58 (s, 2H), 2.65-2.57 (m, 2H), 2.17 (s, 3H), 2.02 (s, |
| | | 3H), 1.87-1.75 (m, 2H), 1.26 (d, J = 2.2 Hz, 6H). |
| 6.73 | 575.9 | 1H NMR (400 MHz, DMSO-d6) δ 8.76 (s, 1H), 8.49 (t, J = 1.9 Hz, |
| | | 1H), 8.44 (d, J = 2.7 Hz, 1H), 8.24 (dd, J = 8.0, 1.7 Hz, 1H), 7.98 (dd, J = |
| | | 7.7, 1.7 Hz, 1H), 7.81 (t, J = 7.8 Hz, 1H), 7.72-7.61 (m, 1H), 6.33 |
| | | (s, 1H), 3.45 (s, 3H), 2.67-2.56 (m, 2H), 2.18 (s, 3H), 2.04 (s, 3H), |
| | | 1.96-1.66 (m, 2H). |
| 6.74 | 594.2 | 1H NMR (400 MHz, DMSO-d6) δ 8.78 (s, 1H), 8.49 (t, J = 1.8 Hz, |
| | | 1H), 8.44 (d, J = 2.7 Hz, 1H), 8.03 (dd, J = 8.4, 7.2 Hz, 1H), 7.76 (dd, J = |
| | | 8.5, 1.1 Hz, 1H), 7.67 (dt, J = 10.4, 2.4 Hz, 1H), 6.32 (s, 1H), 3.49 (s, |
| | | 3H), 2.67-2.56 (m, 2H), 2.17 (s, 3H), 2.02 (s, 3H), 1.91-1.66 (m, 2H). |
| 6.75 | 556.1 | 1H NMR (400 MHz, DMSO-d6) δ 8.67 (d, J = 0.8 Hz, 1H), 8.42 (t, J = |
| | | 1.8 Hz, 1H), 8.37 (d, J = 2.7 Hz, 1H), 8.03 (dd, J = 8.0, 1.4 Hz, 1H), |
| | | 7.69 (dd, J = 7.7, 1.4 Hz, 1H), 7.62-7.53 (m, 2H), 6.30-6.18 (m, 1H), |
| | | 3.24 (s, 3H), 2.59-2.49 (m, 2H), 2.41 (s, 3H), 2.09 (s, 3H), 1.97 (s, |
| | | 3H), 1.81-1.63 (m, 2H). |
| 6.76 | 554.1 | 1H NMR (400 MHz, DMSO-d6) δ 8.74-8.65 (m, 1H), 8.42 (t, J = 1.8 |
| | | Hz, 1H), 8.37 (d, J = 2.7 Hz, 1H), 7.92-7.78 (m, 2H), 7.70-7.53 (m, |
| | | 2H), 6.29-6.21 (m, 1H), 3.55 (t, J = 6.9 Hz, 2H), 3.29 (td, J = 6.6, 3.1 |
| | | Hz, 2H), 2.64-2.50 (m, 2H), 2.09 (s, 3H), 1.98 (s, 3H), 1.86-1.69 (m, 2H). |
| 6.77 | 567.8 | 1H NMR (400 MHz, Methanol-d4) δ 8.80 (d, J = 0.8 Hz, 1H), 8.73 (d, |
| | | J = 4.9 Hz, 1H), 8.10 (t, J = 5.0 Hz, 1H), 7.58 (s, 1H), 6.25 (d, J = 1.0 |
| | | Hz, 1H), 2.76 (ddd, J = 8.8, 5.9, 4.5 Hz, 1H), 2.57 (ddd, J = 9.1, 6.2, 4.5 |
| | | Hz, 1H), 2.43 (s, 6H), 2.30-2.24 (m, 3H), 2.09 (s, 3H), 1.83-1.66 (m, 2H). |
| 6.78 | 583.8 | 1H NMR (400 MHz, Methanol-d4) δ 8.74 (d, J = 0.8 Hz, 1H), 8.14 |
| | | (ddd, J = 9.0, 7.9, 6.1 Hz, 1H), 7.63 (td, J = 8.9, 1.6 Hz, 1H), 7.58 (s, |
| | | 1H), 6.24 (s, 1H), 2.76 (ddd, J = 8.8, 6.0, 4.5 Hz, 1H), 2.57 (ddd, J = |
| | | 9.1, 6.2, 4.5 Hz, 1H), 2.44 (s, 3H), 2.43 (s, 3H), 2.24 (s, 3H), 2.08 (s, |
| | | 3H), 1.77 (ddd, J = 8.8, 6.2, 5.1 Hz, 1H), 1.74-1.66 (m, 1H). |
| 6.79 | 566.8 | 1H NMR (400 MHz, Methanol-d4) δ 8.74 (d, J = 0.8 Hz, 1H), 8.10- |
| | | 8.00 (m, 1H), 7.79 (td, J = 7.5, 6.9, 1.8 Hz, 1H), 7.70 (td, J = 7.9, 0.8 |
| | | Hz, 1H), 7.58 (s, 1H), 6.24 (d, J = 1.0 Hz, 1H), 2.76 (ddd, J = 8.8, 5.9, |
| | | 4.5 Hz, 1H), 2.57 (ddd, J = 9.0, 6.2, 4.5 Hz, 1H), 2.42 (s, 3H), 2.41 (s, |
| | | 3H), 2.25 (s, 3H), 2.08 (s, 3H), 1.77 (ddd, J = 8.8, 6.2, 5.1 Hz, 1H), 1.70 |
| | | (dt, J = 9.0, 5.5 Hz, 1H). |
| 6.80 | 577.7 | 1H NMR (400 MHz, Methanol-d4) δ 8.72 (d, J = 0.8 Hz, 1H), 8.68 (d, J = |
| | | 0.8 Hz, 2H), 7.98-7.91 (m, 1H), 7.80-7.73 (m, 1H), 7.61 (t, J = 7.9 |
| | | Hz, 1H), 6.30 (d, J = 1.0 Hz, 1H), 3.04 (ddd, J = 9.0, 6.2, 4.3 Hz, 1H), |
| | | 2.82 (ddd, J = 8.8, 5.8, 4.3 Hz, 1H), 2.44 (s, 3H), 2.42 (s, 3H), 2.24 (s, |
| | | 3H), 2.10 (s, 3H), 2.01-1.94 (m, 1H), 1.80 (ddd, J = 8.8, 6.2, 4.7 Hz, 1H). |
| 6.81 | 563.8 | 1H NMR (400 MHz, Methanol-d4) δ 8.72 (s, 1H), 8.68 (s, 2H), 8.12 (s, |
| | | 1H), 8.00-7.93 (m, 1H), 7.80 (ddd, J = 8.4, 6.6, 1.8 Hz, 1H), 7.61 (t, J = |
| | | 7.8 Hz, 1H), 6.30 (s, 1H), 3.91 (s, 3H), 3.04 (ddd, J = 8.9, 6.2, 4.3 Hz, |
| | | 1H), 2.82 (ddd, J = 8.8, 5.8, 4.3 Hz, 1H), 2.24 (s, 3H), 2.10 (s, 3H), 1.97 |
| | | (ddd, J = 8.9, 5.8, 4.7 Hz, 1H), 1.80 (ddd, J = 8.8, 6.2, 4.7 Hz, 1H). |
| 6.82 | 565.1 | 1H NMR (400 MHz, DMSO-d6) δ 8.75 (s, 1H), 7.79 (td, J = 7.3, 1.8 |
| | | Hz, 1H), 7.69 (s, 1H), 7.63 (td, J = 7.3, 1.9 Hz, 1H), 7.51 (t, J = 7.7 Hz, |
| | | 1H), 6.25 (d, J = 9.7 Hz, 2H), 3.66 (d, J = 1.0 Hz, 3H), 2.63-2.55 (m, |
| | | 2H), 2.19 (s, 3H), 2.15 (s, 3H), 1.98 (s, 3H), 1.68 (t, J = 7.4 Hz, 2H). |
| 6.83 | 560.1 | 1H NMR (400 MHz, DMSO-d6) δ 8.83 (d, J = 0.8 Hz, 2H), 8.76 (s, |
| | | 1H), 8.02 (dddd, J = 20.2, 8.1, 6.8, 1.8 Hz, 2H), 7.61 (t, J = 7.8 Hz, 1H), |
| | | 6.38-6.31 (m, 1H), 3.28 (s, 3H), 2.86 (tdd, J = 9.6, 6.2, 4.3 Hz, 2H), |
| | | 2.16 (s, 3H), 2.01 (s, 3H), 1.87-1.73 (m, 2H). |
| 6.84 | 566.0 | 1H NMR (400 MHz, DMSO-d6) δ 8.77 (s, 1H), 7.91 (td, J = 7.4, 1.8 |
| | | Hz, 1H), 7.78 (td, J = 7.3, 1.8 Hz, 1H), 7.69 (s, 1H), 7.58 (t, J = 7.7 Hz, |
| | | 1H), 6.26 (s, 1H), 3.72 (d, J = 1.5 Hz, 3H), 2.64-2.54 (m, 2H), 2.30 (s, |
| | | 3H), 2.15 (s, 3H), 1.98 (s, 3H), 1.68 (t, J = 7.4 Hz, 2H). |
| 6.85 | 621.1 | 1H NMR (400 MHz, DMSO) δ 9.63 (d, J = 6.2 Hz, 1H), 8.78 (d, J = 2.0 |
| | | Hz, 1H), 8.20 (s, 1H), 8.14 (q, J = 7.4 Hz, 2H), 7.81 (s, 1H), 7.75 (t, J = |
| | | 59.4 Hz, 1H), 7.68 (td, J = 7.9, 3.3 Hz, 1H), 6.23 (s, 1H), 4.17 (d, J = |
| | | 14.4 Hz, 1H), 3.99 (d, J = 14.3 Hz, 1H), 2.46-2.37 (m, 2H), 2.17 (s, |
| | | 3H), 1.99 (d, J = 7.4 Hz, 3H), 1.65 (m, 2H). |
| 6.86 | 606.2 | 1H NMR (400 MHz, DMSO) δ 8.78 (s, 1H), 8.20 (s, 1H), 8.12-7.97 |
| | | (m, 2H), 7.81 (s, 1H), 7.77 (t, J = 60 Hz, 1H), 7.61 (d, J = 7.8 Hz, 1H), |
| | | 6.24 (s, 1H), 3.86 (s, 1H), 3.71 (dd, J = 12.5, 7.9 Hz, 1H), 2.48-2.37 |
| | | (m, 4H), 2.28 (m, 2H), 2.17 (s, 3H), 2.00 (s, 3H), 1.72-1.56 (m, 2H). |
| 6.87 | 570.8 | 1H NMR (400 MHz, Acetone-d6) δ 7.48 (t, J = 0.9 Hz, 1H), 7.19 (s, |
| | | 1H), 7.12 (d, J = 2.6 Hz, 1H), 6.92-6.84 (m, 1H), 6.40 (d, J = 7.9 Hz, |
| | | 1H), 6.35 (dt, J = 9.9, 2.3 Hz, 1H), 5.07 (s, 1H), 3.79 (d, J = 17.4 Hz, |
| | | 1H), 2.48 (d, J = 6.1 Hz, 3H), 1.55-1.46 (m, 1H), 1.38 (q, J = 6.5, 5.8 |
| | | Hz, 1H), 1.03-0.96 (m, 3H), 0.87 (s, 3H), 0.58 (ddd, J = 9.1, 6.1, 3.8 |
| | | Hz, 2H). |
| 7.1 | 576.1 | 1H NMR (400 MHz, DMSO-d6) δ 9.72 (s, 1H), 8.82 (s, 1H), 7.81- |
| | | 7.72 (m, 1H), 7.72-7.64 (m, 2H), 7.38-7.25 (m, 3H), 7.14 (t, J = 8.9 |
| | | Hz, 2H), 6.23-6.12 (m, 1H), 4.84 (d, J = 6.1 Hz, 2H), 4.36 (d, J = 6.1 |
| | | Hz, 2H), 2.55-2.45 (m, 2H), 2.07 (s, 3H), 1.97-1.84 (m, 3H), 1.67 (t, J = |
| | | 7.6 Hz, 2H), 1.63 (s, 3H). |
| 7.2 | 562.1 | 1H NMR (400 MHz, DMSO-d6) δ 9.85 (s, 1H), 8.81 (s, 1H), 7.96 (t, J = |
| | | 7.5 Hz, 1H), 7.68 (d, J = 5.8 Hz, 2H), 7.36-7.27 (m, 3H), 7.15 (t, J = |
| | | 8.8 Hz, 2H), 6.19 (s, 1H), 4.70 (dq, J = 9.1, 5.8 Hz, 4H), 4.09 (p, J = 7.3 |
| | | Hz, 1H), 2.55-2.45 (m, 2H), 2.06 (s, 3H), 1.94 (s, 3H), 1.67 (t, J = 7.4 |
| | | Hz, 2H). |
| 7.3 | 596.1 | 1H NMR (400 MHz, DMSO-d6) δ 9.30 (s, 1H), 8.82 (s, 1H), 7.81 (t, J = |
| | | 7.5 Hz, 1H), 7.71 (s, 1H), 7.57 (t, J = 7.3 Hz, 1H), 7.36-7.24 (m, |
| | | 3H), 7.14 (t, J = 8.8 Hz, 2H), 6.52 (t, J = 56.7 Hz, 1H), 6.19 (s, 1H), |
| | | 2.55-2.45 (m, 2H), 2.06 (s, 3H), 1.93 (s, 3H), 1.67 (t, J = 7.4 Hz, 2H), |
| | | 1.35 (s, 2H), 1.21-1.09 (m, 2H). |
| 7.4 | 590.1 | 1H NMR (400 MHz, DMSO-d6) δ 9.45 (s, 1H), 8.82 (s, 1H), 7.78 (t, J = |
| | | 7.0 Hz, 1H), 7.70 (d, J = 8.7 Hz, 2H), 7.37-7.25 (m, 3H), 7.14 (t, J = |
| | | 8.8 Hz, 2H), 6.19 (s, 1H), 3.21 (s, 3H), 2.55-2.45 (m, 2H), 2.41 (m, |
| | | 2H), 2.27-2.13 (m, 2H), 2.06 (s, 3H), 1.98-1.88 (m, 3H), 1.87-1.58 |
| | | (m, 4H). |
| 8.1 | 564.1 | 1H NMR (400 MHz, DMSO-d6) δ 10.12 (d, J = 1.9 Hz, 1H), 8.82 (s, |
| | | 1H), 7.88-7.78 (m, 1H), 7.70 (d, J = 1.5 Hz, 1H), 7.62-7.53 (m, 1H), |
| | | 7.39-7.27 (m, 3H), 7.20-7.07 (m, 2H), 6.19 (s, 1H), 2.55-2.45(m, |
| | | 2H), 2.07 (s, 3H), 1.97-1.88 (m, 3H), 1.67 (t, J = 7.4 Hz, 2H), 1.50- |
| | | 1.39 (m, 2H), 1.30 (td, J = 9.3, 8.8, 5.5 Hz, 2H). |
| 8.2 | 561.1 | 1H NMR (400 MHz, DMSO-d6) δ 9.12 (s, 1H), 8.82 (s, 1H), 8.49 (s, |
| | | 1H), 8.43 (d, J = 2.7 Hz, 1H), 7.83-7.73 (m, 1H), 7.73-7.61 (m, 2H), |
| | | 7.55 (t, J = 7.6 Hz, 1H), 7.30 (t, J = 7.9 Hz, 1H), 6.23 (s, 1H), 2.60 (t, J = |
| | | 7.5 Hz, 2H), 2.07 (d, J = 2.9 Hz, 3H), 1.94 (s, 3H), 1.86-1.70 (m, |
| | | 2H), 1.41 (d, J = 1.4 Hz, 3H), 1.09 (q, J = 3.6 Hz, 2H), 0.66 (q, J = 3.7 |
| | | Hz, 2H). |
| 9.1 | 523.1 | 1H NMR (400 MHz, DMSO-d6) δ 8.82 (s, 1H), 8.31 (d, J = 4.2 Hz, |
| | | 1H), 8.00 (td, J = 7.6, 2.1 Hz, 1H), 7.82-7.70 (m, 1H), 7.51-7.37 (m, |
| | | 2H), 6.07-6.03 (m, 1H), 3.01 (s, 3H), 2.88 (s, 3H), 2.74-2.64 (m, |
| | | 1H), 2.58-2.52 (m, 1H), 2.04 (d, J = 6.7 Hz, 3H), 2.02-1.94 (m, 1H), |
| | | 1.93-1.85 (m, 3H), 1.56-1.33 (m, 2H), 0.68-0.58 (m, 2H), 0.47- |
| | | 0.36 (m, 2H). |
| 9.2 | 559.1 | 1H NMR (400 MHz, DMSO-d6) δ 10.37 (s, 1H), 8.84 (s, 1H), 8.01 (td, |
| | | J = 7.6, 2.1 Hz, 1H), 7.80 (d, J = 1.9 Hz, 1H), 7.62 (d, 2H), 7.54-7.38 |
| | | (m, 2H), 7.31 (t, 2H), 7.06 (t, J = 7.4, 1.2 Hz, 1H), 6.16 (s, 1H), 3.02 (s, |
| | | 3H), 2.88 (d, J = 1.0 Hz, 3H), 2.72-2.61 (m, 1H), 2.36-2.26 (m, 1H), |
| | | 2.05 (s, 3H), 1.93 (s, 3H), 1.68-1.52 (m, 2H). |
| 9.3 | 601.1 | 1H NMR (400 MHz, DMSO-d6) δ 8.83 (s, 1H), 8.00 (td, J = 7.5, 2.1 |
| | | Hz, 1H), 7.77 (s, 1H), 7.52-7.33 (m, 2H), 6.19 (s, 1H), 4.18-4.07 (m, |
| | | 2H), 3.99-3.85 (m, 2H), 3.38-3.08 (m, 4H), 3.01 (s, 3H), 2.88 (s, |
| | | 3H), 2.81-2.65 (m, 2H), 2.06 (s, 3H), 1.92 (s, 3H), 1.58-1.34 (m, 2H). |
| 10.1 | 563.9 | 1H NMR (400 MHz, DMSO-d6) δ 8.85 (s, 1H), 8.04 (d, J = 5.2 Hz, |
| | | 1H), 7.89 (s, 3H), 7.80 (s, 1H), 7.31 (m, 2H), 7.23 (td, J = 5.1, 1.3 Hz, |
| | | 1H), 7.20-7.10 (m, 2H), 6.49 (s, 1H), 6.21 (d, J = 2.4 Hz, 1H), 4.20 (d, |
| | | J = 9.2 Hz, 2H), 4.01 (d, J = 8.9 Hz, 2H), 3.26-3.16 (m, 2H), 2.49- |
| | | 2.43 (m, 2H), 2.08 (s, 3H), 1.93 (s, 3H), 1.77-1.63 (m, 2H). |
| 10.2 | 547.9 | 1H NMR (400 MHz, DMSO-d6) δ 8.86 (s, 1H), 8.38 (s, 3H), 8.06 (d, J = |
| | | 5.2 Hz, 1H), 7.82 (s, 1H), 7.38-7.25 (m, 3H), 7.21-7.09 (m, 2H), |
| | | 6.21 (d, J = 3.0 Hz, 1H), 4.21 (d, J = 9.3 Hz, 2H), 4.15-4.03 (m, 2H), |
| | | 2.49-2.40 (m, 2H), 2.08 (s, 3H), 1.92 (s, 3H), 1.79-1.63 (m, 2H), |
| | | 1.60 (s, 3H). |
| 10.3 | 549.9 | 1H NMR (400 MHz, Methanol-d4) δ 8.90 (s, 1H), 8.50-8.42 (m, 1H), |
| | | 8.39 (d, J = 2.6 Hz, 1H), 7.92-7.79 (m, 2H), 7.67-7.55 (m, 1H), 7.43 |
| | | (t, J = 6.0 Hz, 1H), 6.30 (s, 1H), 4.43-4.24 (m, 4H), 2.81-2.70 (m, |
| | | 1H), 2.68-2.56 (m, 1H), 2.20 (s, 3H), 2.05 (s, 3H), 1.88-1.76 (m, |
| | | 2H), 1.61 (s, 3H). |
| 10.4 | 548.9 | 1H NMR (400 MHz, Methanol-d4) δ 8.85 (s, 1H), 8.44 (t, J = 1.6 Hz, |
| | | 1H), 8.38 (d, J = 2.6 Hz, 1H), 8.04 (d, J = 5.3 Hz, 1H), 7.77 (s, 1H), |
| | | 7.62-7.54 (m, 1H), 7.32 (t, J = 5.2 Hz, 1H), 6.29 (s, 1H), 4.33-4.18 |
| | | (m, 4H), 2.82-2.69 (m, 1H), 2.69-2.56 (m, 1H), 2.19 (s, 3H), 2.05 (s, |
| | | 3H), 1.89-1.77 (m, 2H), 1.71 (s, 3H). |
| 10.5 | 645.9 | 1H NMR (400 MHz, Methanol-d4) δ 8.73 (s, 1H), 8.49 (s, 1H), 8.44 (d, |
| | | J = 2.6 Hz, 1H), 8.14 (dd, J = 5.2, 1.9 Hz, 1H), 7.70 (dt, J = 9.6, 2.3 Hz, |
| | | 1H), 7.38 (d, J = 4.9 Hz, 1H), 6.33 (s, 1H), 4.26 (dd, J = 12.2, 5.5 Hz, |
| | | 1H), 4.21-4.08 (m, 1H), 4.08-3.90 (m, 2H), 3.86 (t, J = 11.2 Hz, |
| | | 1H), 2.78 (ddd, J = 8.8, 6.3, 4.6 Hz, 1H), 2.71-2.59 (m, 1H), 2.25 (s, |
| | | 3H), 2.15-2.07 (m, 6H), 1.86 (ddd, J = 8.6, 6.2, 4.4 Hz, 2H). |
| 10.6 | 623.9 | 1H NMR (400 MHz, Methanol-d4) δ 8.71 (s, 1H), 8.45 (s, 1H), 8.37 (d, |
| | | J = 2.7 Hz, 1H), 8.10 (t, J = 4.9 Hz, 1H), 7.60 (d, J = 9.9 Hz, 1H), 7.30- |
| | | 7.19 (m, 1H), 6.32 (s, 1H), 4.27 (t, J = 11.2 Hz, 1H), 3.81 (d, J = 11.7 |
| | | Hz, 1H), 3.77-3.67 (m, 1H), 3.60 (d, J = 13.5 Hz, 2H), 2.83-2.69 (m, |
| | | 1H), 2.63 (s, 1H), 2.24 (s, 3H), 2.11-2.01 (m, 7H), 1.85 (s, 3H), 0.14- |
| | | 0.09 (m, 2H). |
| 10.7 | 610.9 | 1H NMR (400 MHz, Methanol-d4) δ 8.72 (s, 1H), 8.47 (s, 1H), 8.40 (d, |
| | | J = 2.6 Hz, 1H), 8.08 (d, J = 5.2 Hz, 1H), 7.64 (d, J = 9.9 Hz, 1H), 7.28 |
| | | (t, J = 4.7 Hz, 1H), 6.33 (s, 1H), 4.58 (d, J = 9.8 Hz, 1H), 4.39 (d, J = |
| | | 9.9 Hz, 1H), 4.29 (d, J = 11.1 Hz, 1H), 4.15 (d, J = 10.8 Hz, 1H), 2.81- |
| | | 2.72 (m, 1H), 2.64 (m, 1H), 2.25 (s, 3H), 2.10 (s, 3H), 1.94 (s, 3H), |
| | | 1.91-1.78 (m, 5H). |
| 10.8 | 595.9 | 1H NMR (400 MHz, Methanol-d4) δ 8.73 (s, 1H), 8.46 (s, 1H), 8.39 (d, |
| | | J = 2.6 Hz, 1H), 8.08 (d, J = 5.1 Hz, 1H), 7.62 (dt, J = 9.7, 2.4 Hz, 1H), |
| | | 7.31 (t, J = 4.7 Hz, 1H), 6.33 (s, 1H), 5.55 (ddd, J = 10.6, 6.8, 4.0 Hz, |
| | | 1H), 4.70 (dd, J = 10.1, 6.7 Hz, 1H), 4.47 (dd, J = 11.2, 6.7 Hz, 1H), |
| | | 4.33 (d, J = 9.8 Hz, 1H), 4.08 (d, J = 11.3 Hz, 1H), 2.80-2.72 (m, 1H), |
| | | 2.65 (t, J = 10.0 Hz, 1H), 2.25 (s, 3H), 2.11 (s, 3H), 1.94 (s, 3H), 1.84 |
| | | (ddd, J = 8.4, 6.2, 4.1 Hz, 2H).- |
| 10.9 | 539.9 | 1H NMR (400 MHz, DMSO-d6) δ 8.75 (s, 1H), 8.55-8.36 (m, 2H), |
| | | 7.96 (d, J = 5.2 Hz, 1H), 7.67 (d, J = 10.4 Hz, 1H), 6.76-6.54 (m, 2H), |
| | | 6.32 (s, 1H), 4.35-4.14 (m, 1H), 2.62 (t, J = 7.5 Hz, 2H), 2.15 (s, 3H), |
| | | 2.00 (s, 3H), 1.88-1.76 (m, 2H), 1.27-1.12 (m, 6H). |
| 10.10 | 579.9 | 1H NMR (400 MHz, DMSO-d6) δ 8.76 (d, J = 0.8 Hz, 1H), 8.49 (t, J = |
| | | 1.9 Hz, 1H), 8.44 (d, J = 2.6 Hz, 1H), 8.06 (d, J = 5.2 Hz, 1H), 7.67 (dt, |
| | | J = 10.3, 2.4 Hz, 1H), 6.92 (t, J = 4.6 Hz, 1H), 6.32 (s, 1H), 4.88 (s, |
| | | 1H), 4.63 (s, 1H), 3.85-3.50 (m, 3H), 3.42 (dd, J = 10.5, 4.5 Hz, 1H), |
| | | 2.67-2.58 (m, 2H), 2.16 (s, 3H), 2.00 (s, 3H), 1.94 (dd, J = 9.6, 2.3 |
| | | Hz, 1H), 1.89-1.76 (m, 3H). |
| 10.11 | 579.9 | 1H NMR (400 MHz, DMSO-d6) δ 8.76 (s, 1H), 8.55-8.36 (m, 2H), |
| | | 8.06 (d, J = 5.0 Hz, 1H), 7.73-7.60 (m, 1H), 6.92 (t, J = 4.6 Hz, 1H), |
| | | 6.32 (s, 1H), 4.88 (s, 1H), 4.63 (s, 1H), 4.03-3.49 (m, 3H), 3.41 (dd, J = |
| | | 10.3, 4.6 Hz, 1H), 2.62 (t, J = 7.4 Hz, 2H), 2.16 (s, 3H), 1.99 (s, 3H), |
| | | 1.97-1.89 (m, 1H), 1.89-1.73 (m, 3H) |
| 11.1 | 561.2 | 1H NMR (400 MHz, DMSO-d6) δ 9.84 (s, 1H), 8.89 (s, 1H), 8.38 (d, J = |
| | | 5.0 Hz, 1H), 7.89 (t, J = 5.2 Hz, 1H), 7.85 (s, 1H), 7.35-7.27 (m, |
| | | 2H), 7.20-7.10 (m, 2H), 6.19 (s, 1H), 2.09 (s, 3H), 1.93 (s, 3H), 1.73- |
| | | 1.54 (m, 2H), 1.40 (s, 3H), 1.35-1.07 (m, 3H), 0.93-0.84 (m, 1H), |
| | | 0.71-0.65 (m, 2H). |
| 12.1 | 500.1 | 1H NMR (400 MHz, DMSO-d6) δ 8.80 (s, 1H), 8.01 (td, J = 7.6, 2.2 |
| | | Hz, 1H), 7.70 (d, J = 1.9 Hz, 1H), 7.55-7.36 (m, 2H), 7.36-7.19 (m, |
| | | 2H), 7.19-7.08 (m, 2H), 6.23 (dd, J = 21.9, 6.2 Hz, 2H), 3.02 (s, 3H), |
| | | 2.89 (s, 3H), 2.48-2.35 (m, 1H), 2.15-2.02 (m, 4H), 1.91 (s, 3H), |
| | | 1.64-1.47 (m, 2H). |
| 13.1 | 580.0 | 1H NMR (400 MHz, DMSO-d6) δ 8.83 (s, 1H), 8.02 (td, J = 7.5, 2.1 |
| | | Hz, 1H), 7.78 (s, 1H), 7.55-7.37 (m, 2H), 7.37-7.23 (m, 2H), 7.22- |
| | | 7.07 (m, 2H), 6.18 (s, 1H), 3.02 (s, 3H), 2.89 (s, 3H), 2.49-2.42 (m, |
| | | 2H), 2.07 (s, 3H), 1.93 (s, 3H), 1.75-1.61 (m, 2H). |
| 14.1 | 553.1 | 1H NMR (400 MHz, DMSO-d6) δ 9.44 (s, 1H), 8.81 (s, 1H), 8.56 (d, J = |
| | | 1.9 Hz, 1H), 8.48 (d, J = 2.2 Hz, 1H), 7.86 (t, J = 2.2 Hz, 1H), 7.72 (t, |
| | | J = 7.6 Hz, 1H), 7.70-7.58 (m, 2H), 7.29 (t, J = 7.9 Hz, 1H), 6.23 (s, |
| | | 1H), 3.68 (s, 3H), 2.64-2.55 (m, 2H), 2.06 (s, 3H), 1.94 (s, 3H), 1.83 |
| | | (dt, J = 8.6, 5.7 Hz, 1H), 1.76 (dt, J = 9.0, 5.7 Hz, 1H). |
| 14.2 | 566.1 | 1H NMR (400 MHz, DMSO-d6) δ 8.81 (s, 1H), 8.55 (d, J = 1.9 Hz, |
| | | 1H), 8.48 (d, J = 2.2 Hz, 1H), 8.10 (s, 1H), 7.86 (t, J = 2.1 Hz, 1H), 7.69- |
| | | 7.59 (m, 2H), 7.59-7.44 (m, 1H), 7.24 (t, J = 7.9 Hz, 1H), 6.23 (s, |
| | | 1H), 2.93 (s, 6H), 2.60 (m, 2H), 2.06 (d, J = 3.0 Hz, 3H), 1.94 (s, 3H), |
| | | 1.87-1.79 (m, 1H), 1.75 (dt, J = 8.9, 5.7 Hz, 1H). |
| 14.3 | 565.1 | 1H NMR (400 MHz, DMSO-d6) δ 8.83 (s, 1H), 8.56 (d, J = 1.9 Hz, |
| | | 1H), 8.48 (d, J = 2.3 Hz, 1H), 7.91-7.82 (m, 2H), 7.75 (d, J = 1.7 Hz, |
| | | 1H), 7.63 (ddd, J = 8.9, 7.3, 1.8 Hz, 1H), 7.40 (t, J = 7.9 Hz, 1H), 6.24 |
| | | (s, 1H), 4.50 (t, J = 7.9 Hz, 2H), 4.05 (td, J = 7.7, 2.0 Hz, 2H), 2.64- |
| | | 2.55 (m, 2H), 2.07 (d, J = 2.4 Hz, 3H), 1.94 (s, 3H), 1.83 (dt, J = 8.7, |
| | | 5.9 Hz, 1H), 1.76 (dt, J = 8.9, 5.7 Hz, 1H). |
| 15.1 | 591.1 | 1H NMR (400 MHz, DMSO-d6) δ 8.84 (s, 1H), 8.56 (d, J = 1.9 Hz, |
| | | 1H), 8.48 (d, J = 2.3 Hz, 1H), 7.95 (t, J = 7.3 Hz, 1H), 7.86 (t, J = 2.2 |
| | | Hz, 1H), 7.78 (d, J = 1.8 Hz, 1H), 7.57 (t, J = 7.4 Hz, 1H), 7.40 (t, J = |
| | | 7.9 Hz, 1H), 6.24 (s, 1H), 3.21 (s, 3H), 2.63-2.55 (m, 2H), 2.07 (s, |
| | | 3H), 1.94 (s, 3H), 1.83 (dt, J = 8.5, 5.7 Hz, 1H), 1.75 (dt, J = 9.0, 5.7 |
| | | Hz, 1H), 0.98 (m, J = 5.6 Hz, 5H), 0.42 (br s, 2H). |
| 16.1 | 617.1 | 1H NMR (400 MHz, DMSO-d6) δ 8.83 (s, 1H), 8.50 (d, J = 4.2 Hz, |
| | | 1H), 8.01 (td, J = 7.6, 2.1 Hz, 1H), 7.81-7.73 (m, 1H), 7.60-7.51 (m, |
| | | 1H), 7.50-7.38 (m, 3H), 7.37-7.26 (m, 1H), 6.26-6.19 (m, 1H), |
| | | 3.02 (s, 3H), 2.93-2.79 (m, 4H), 2.62-2.52 (m, 1H), 2.07 (s, 3H), |
| | | 1.94 (s, 3H), 1.88-1.68 (m, 2H), 0.75-0.66 (m, 2H), 0.62-0.53 (m, 2H). |
| 17.1 | 665.0 | 1H NMR (400 MHz, DMSO-d6) δ 8.76 (s, 1H), 8.37 (t, J = 1.6 Hz, |
| | | 1H), 8.33 (t, J = 1.5 Hz, 1H), 7.94-7.82 (m, 1H), 7.72 (t, J = 6.9 Hz, |
| | | 1H), 7.58-7.41 (m, 2H), 6.29 (s, 1H), 5.12-4.97 (m, 1H), 4.15-4.05 |
| | | (m, 1H), 3.93-3.83 (m, 1H), 2.71-2.58 (m, 3H), 2.33-2.21 (m, 1H), |
| | | 2.15 (s, 3H), 2.00 (s, 3H), 1.92-1.65 (m, 2H). |
| 17.2 | 575.8 | 1H NMR (400 MHz, DMSO-d6) δ 8.79 (s, 1H), 8.41-8.35 (m, 1H), |
| | | 8.31-8.24 (m, 1H), 8.15-8.08 (m, 1H), 8.08-8.02 (m, 1H), 7.67 (t, J = |
| | | 7.8 Hz, 1H), 7.36 (dt, J = 8.9, 1.7 Hz, 1H), 6.30 (s, 1H), 3.39 (s, 3H), |
| | | 2.68-2.59 (m, 1H), 2.59-2.52 (m, 1H), 2.17 (s, 3H), 2.02 (s, 3H), |
| | | 1.88-1.76 (m, 2H). |
| 17.3 | 591.8 | 1H NMR (400 MHz, DMSO-d6) δ 8.79 (d, J = 0.6 Hz, 1H), 8.37 (t, J = |
| | | 1.6 Hz, 1H), 8.33 (t, J = 1.5 Hz, 1H), 8.15-8.01 (m, 2H), 7.67 (t, J = |
| | | 7.8 Hz, 1H), 7.48 (t, J = 1.6 Hz, 1H), 6.30 (s, 1H), 3.39 (s, 3H), 2.63 |
| | | (m, 1H), 2.59-2.53 (m, 1H), 2.17 (s, 3H), 2.02 (s, 3H), 1.89-1.76 (m, 2H). |
| 17.4 | 608.0 | 1H NMR (400 MHz, DMSO-d6) δ 8.79 (d, J = 0.8 Hz, 1H), 8.45 (s, |
| | | 1H), 8.38-8.29 (m, 1H), 8.14-7.98 (m, 2H), 7.66 (t, J = 7.8 Hz, 1H), |
| | | 7.49 (s, 1H), 7.04 (t, J = 54.9 Hz, 1H), 6.31 (s, 1H), 3.38 (s, 3H), 2.70- |
| | | 2.57 (m, 2H), 2.17 (s, 3H), 2.02 (s, 3H), 1.94-1.83 (m, 1H), 1.83- |
| | | 1.68 (m, 1H). |
| 17.5 | 625.8 | 1H NMR (400 MHz, DMSO-d6) δ 8.79 (d, J = 0.6 Hz, 1H), 8.58 (d, J = |
| | | 7.0 Hz, 2H), 8.13-8.08 (m, 1H), 8.08-8.02 (m, 1H), 7.73-7.62 (m, |
| | | 2H), 6.32 (s, 1H), 3.44 (s, 3H), 2.76-2.60 (m, 2H), 2.17 (s, 3H), 2.03 |
| | | (s, 3H), 1.97-1.88 (m, 1H), 1.87-1.77 (m, 1H). |
| 18.1 | 561.2 | 1H NMR (400 MHz, DMSO-d6) δ 8.82 (d, J = 0.8 Hz, 1H), 8.49 (t, J = |
| | | 1.8 Hz, 1H), 8.44 (d, J = 2.7 Hz, 1H), 8.12 (ddd, J = 8.1, 6.8, 1.8 Hz, |
| | | 1H), 8.06 (ddd, J = 8.3, 6.7, 1.8 Hz, 1H), 7.73-7.68 (m, 1H), 7.68 (t, J = |
| | | 7.8 Hz, 1H), 3.39 (s, 3H), 2.86-2.71 (m, 2H), 2.26 (s, 3H), 2.24 (s, |
| | | 3H), 1.93-1.86 (m, 1H), 1.86-1.78 (m, 1H). |
| 18.2 | 550.2 | 1H NMR (400 MHz, DMSO-d6) δ 8.82 (d, J = 0.8 Hz, 1H), 8.11 (ddd, |
| | | J = 8.2, 6.7, 1.8 Hz, 1H), 8.06 (ddd, J = 8.3, 6.7, 1.8 Hz, 1H), 7.72 (s, |
| | | 1H), 7.67 (t, J = 7.8 Hz, 1H), 3.39 (s, 3H), 2.80-2.74 (m, 1H), 2.69 |
| | | (ddd, J = 8.4, 5.3, 4.0 Hz, 1H), 2.26 (s, 3H), 2.22 (s, 3H), 1.79 (ddd, J = |
| | | 9.2, 5.4, 3.9 Hz, 1H), 1.68 (ddd, J = 8.4, 6.3, 3.8 Hz, 1H). |
| 18.3 | 566.8 | 1H NMR (400 MHz, Methanol-d4) δ 8.75 (d, J = 0.8 Hz, 1H), 7.96 |
| | | (ddd, J = 8.0, 6.5, 1.8 Hz, 1H), 7.77 (ddd, J = 8.7, 7.1, 1.8 Hz, 1H), 7.62 |
| | | (td, J = 7.9, 0.9 Hz, 1H), 7.56 (s, 1H), 2.83 (dtd, J = 8.6, 4.5, 3.8, 1.9 |
| | | Hz, 2H), 2.48-2.41 (m, 6H), 2.33-2.28 (m, 3H), 2.26 (s, 3H), 1.89 |
| | | (ddd, J = 9.2, 5.6, 3.9 Hz, 1H), 1.72 (ddd, J = 8.2, 6.5, 3.9 Hz, 1H). |
| 18.4 | 568.0 | 1H NMR (400 MHz, DMSO-d6) δ 8.86 (d, J = 0.8 Hz, 1H), 8.64 (d, J = |
| | | 4.9 Hz, 1H), 7.99 (t, J = 4.9 Hz, 1H), 7.71 (s, 1H), 2.75 (ddd, J = 9.0, |
| | | 6.2, 4.0 Hz, 1H), 2.69 (ddd, J = 8.4, 5.4, 4.1 Hz, 1H), 2.46 (s, 3H), 2.30 |
| | | (s, 3H), 2.27 (s, 3H), 2.21 (s, 3H), 1.78 (ddd, J = 9.1, 5.4, 3.8 Hz, 1H), |
| | | 1.67 (ddd, J = 8.4, 6.3, 3.9 Hz, 1H). |
| 18.5 | 567.2 | 1H NMR (400 MHz, DMSO) δ 8.82 (s, 1H), 8.15-8.08 (m, 1H), 8.08- |
| | | 8.01 (m, 1H), 7.67 (t, J = 7.8 Hz, 1H), 7.37 (d, J = 3.3 Hz, 1H), 3.39 |
| | | (s, 3H), 2.58-2.51 (m, 1H), 2.43-2.35 (m, 1H), 2.27 (s, 3H), 2.21 (s, |
| | | 3H), 1.73-1.63 (m, 1H), 1.59-1.47 (m, 1H). |
| 18.6 | 549.2 | 1H NMR (400 MHz, DMSO-d6) δ 8.82 (d, J = 0.8 Hz, 1H), 8.15-8.09 |
| | | (m, 1H), 8.09-7.99 (m, 1H), 7.67 (t, J = 7.8 Hz, 1H), 7.63 (d, J = 0.8 |
| | | Hz, 1H), 7.38 (d, J = 0.8 Hz, 1H), 3.39 (s, 3H), 2.56-2.49 (m, 1H), |
| | | 2.47-2.40 (m, 1H), 2.26 (s, 3H), 2.20 (s, 3H), 1.76-1.68 (m, 1H), |
| | | 1.58-1.45 (m, 1H). |
| 18.7 | 549.2 | 1H NMR (400 MHz, DMSO-d6) δ 8.80 (s, 1H), 8.06 (td, J = 7.4, 1.8 |
| | | Hz, 1H), 8.00 (td, J = 8.2, 6.5, 1.8 Hz, 1H), 7.72 (s, 1H), 7.61 (t, J = 7.8 |
| | | Hz, 1H), 3.25 (s, 3H), 2.80-2.73 (m, 1H), 2.73-2.65 (m, 1H), 2.25 (s, |
| | | 3H), 2.22 (s, 3H), 1.84-1.73 (m, 1H), 1.72-1.61 (m, 1H). |
| 18.8 | 562.1 | 1H NMR (400 MHz, DMSO-d6) δ 8.86-8.82 (m, 2H), 8.81 (s, 1H), |
| | | 8.14-7.98 (m, 2H), 7.67 (t, J = 7.8 Hz, 1H), 3.39 (s, 3H), 3.06-2.88 |
| | | (m, 2H), 2.25 (s, 3H), 2.23 (s, 3H), 1.96-1.71 (m, 2H). |
| 18.9 | 561.1 | 1H NMR (400 MHz, DMSO-d6) δ 8.81 (s, 1H), 8.57 (d, J = 5.1 Hz, |
| | | 1H), 8.15-8.01 (m, 2H), 7.67 (t, J = 7.8 Hz, 1H), 7.24 (d, J = 5.1 Hz, |
| | | 1H), 3.39 (s, 3H), 2.97-2.87 (m, 2H), 2.25 (s, 3H), 2.23 (s, 3H), 1.89- |
| | | 1.78 (m, 2H). |
| 19.1 | 568.0 | 1H NMR (400 MHz, DMSO-d6) δ 8.77 (s, 1H), 8.40 (dd, J = 8.7, 3.4 |
| | | Hz, 1H), 8.27 (dd, J = 9.6, 8.7 Hz, 1H), 7.71 (s, 1H), 2.71 (dddd, J = |
| | | 23.5, 8.5, 5.8, 4.1 Hz, 2H), 2.47 (s, 3H), 2.30 (s, 3H), 2.22 (s, 3H), 2.19 |
| | | (s, 3H), 1.78 (ddd, J = 9.3, 5.5, 4.0 Hz, 1H), 1.67 (ddd, J = 8.4, 6.3, 3.8 |
| | | Hz, 1H). |
| 19.2 | 578.8 | 1H NMR (400 MHz, Methanol-d4) δ 8.71 (s, 1H), 8.47 (m, 3H), 8.15 |
| | | (t, J = 9.1 Hz, 1H), 7.71 (d, J = 9.7 Hz, 1H), 6.33 (s, 1H), 2.78 (dt, J = |
| | | 8.8, 5.8 Hz, 1H), 2.66 (q, J = 6.7 Hz, 1H), 2.60 (s, 3H), 2.41 (s, 3H), |
| | | 2.24 (s, 3H), 2.11 (s, 3H), 1.86 (tt, J = 9.6, 5.9 Hz, 2H). |
| 19.3 | 598.8 | 1H NMR (400 MHz, Methanol-d4) δ 8.79-8.65 (m, 1H), 8.58-8.38 |
| | | (m, 1H), 8.27-8.05 (m, 1H), 8.01 (s, 1H), 7.72 (s, 1H), 7.44 (t, J = |
| | | 59.8 Hz, 1H), 6.27-6.16 (m, 1H), 2.59 (dt, J = 9.0, 5.6 Hz, 4H), 2.50- |
| | | 2.34 (m, 4H), 2.24 (s, 3H), 2.10 (s, 3H), 1.77-1.57 (m, 2H). |
| 19.4 | 580.8 | 1H NMR (400 MHz, Methanol-d4) δ 8.26 (s, 1H), 8.01 (s, 1H), 7.72 (s, |
| | | 1H), 7.70-7.63 (m, 2H), 7.61-7.54 (m, 1H), 6.24 (s, 1H), 2.84 (s, |
| | | 3H), 2.60 (ddd, J = 8.6, 5.7, 4.5 Hz, 1H), 2.42 (m, 4H), 2.27 (s, 3H), |
| | | 2.10 (s, 3H), 1.74-1.62 (m, 3H). |
| 19.5 | 549.8 | 1H NMR (400 MHz, Methanol-d4) δ 8.71 (s, 1H), 8.24 (t, J = 7.4 Hz, |
| | | 1H), 8.03 (d, J = 16.1 Hz, 1H), 7.59 (s, 1H), 6.25 (s, 1H), 2.94-2.84 |
| | | (m, 3H), 2.77 (ddd, J = 8.7, 5.9, 4.5 Hz, 1H), 2.58 (ddd, J = 9.0, 6.2, 4.5 |
| | | Hz, 1H), 2.50-2.38 (m, 4H), 2.25 (s, 3H), 2.10 (s, 3H), 1.85-1.65 (m, 2H). |
| 19.6 | 566.8 | 1H NMR (400 MHz, Methanol-d4) δ 8.70 (s, 1H), 8.48 (dd, J = 9.3, 3.0 |
| | | Hz, 1H), 8.14 (t, J = 9.0 Hz, 1H), 7.58 (s, 1H), 6.25 (s, 1H), 2.76 (ddd, J = |
| | | 8.7, 5.9, 4.5 Hz, 1H), 2.62 (s, 3H), 2.57 (ddd, J = 9.1, 6.3, 4.5 Hz, |
| | | 1H), 2.43 (s, 3H), 2.23 (s, 3H), 2.09 (s, 3H), 1.78 (ddd, J = 8.7, 6.2, 5.1 |
| | | Hz, 1H), 1.74-1.68 (m, 1H). |
| 20.1 | 594.0 | 1H NMR (400 MHz, DMSO-d6) δ 8.77 (s, 1H), 8.20 (s, 1H), 8.09- |
| | | 7.94 (m, 2H), 7.92-7.56 (m, 3H), 6.22 (s, 1H), 3.32 (s, 3H), 2.46- |
| | | 2.35 (m, 2H), 2.16 (s, 3H), 1.99 (s, 3H), 1.64 (t, J = 7.4 Hz, 2H). |
| 20.2 | 594.0 | 1H NMR (400 MHz, DMSO-d6) δ 8.77 (s, 1H), 8.20 (s, 1H), 8.09- |
| | | 7.94 (m, 2H), 7.92-7.55 (m, 3H), 6.23 (s, 1H), 3.30 (s, 3H), 2.41 (td, J = |
| | | 7.4, 3.5 Hz, 2H), 2.16 (s, 3H), 2.00 (s, 3H), 1.64 (t, J = 7.4 Hz, 2H). |
| 20.3 | 620.0 | 1H NMR (400 MHz, DMSO-d6) δ 8.77 (s, 1H), 8.20 (s, 1H), 8.10- |
| | | 7.98 (m, 2H), 7.92-7.58 (m, 3H), 6.22 (s, 1H), 3.28 (s, 3H), 2.46- |
| | | 2.35 (m, 2H), 2.35-2.20 (m, 1H), 2.16 (s, 3H), 1.99 (s, 3H), 1.64 (dd, J = |
| | | 8.5, 6.2 Hz, 2H), 0.46-0.29 (m, 2H), 0.24-0.03 (m, 2H). |
| 20.4 | 620.0 | 1H NMR (400 MHz, DMSO-d6) δ 8.78 (s, 1H), 8.20 (s, 1H), 8.11- |
| | | 7.99 (m, 2H), 7.95-7.55 (m, 3H), 6.24 (s, 1H), 3.27 (s, 3H), 2.45- |
| | | 2.37 (m, 2H), 2.37-2.27 (m, 1H), 2.16 (s, 3H), 2.00 (s, 3H), 1.65 (dd, J = |
| | | 8.5, 5.9 Hz, 2H), 0.44-0.30 (m, 2H), 0.25-0.05 (m, 2H). |
| 21.1 | 565.1 | 1H NMR (400 MHz, DMSO-d6) δ 8.94 (d, J = 5.1 Hz, 1H), 8.78 (s, |
| | | 1H), 8.50 (t, J = 1.8 Hz, 1H), 8.44 (d, J = 2.7 Hz, 1H), 8.21 (s, 1H), 7.95 |
| | | (d, J = 5.1 Hz, 1H), 7.72-7.62 (m, 1H), 6.33 (d, J = 1.0 Hz, 1H), 2.63 |
| | | (t, J = 7.5 Hz, 2H), 2.17 (s, 3H), 2.01 (s, 3H), 1.89-1.79 (m, 2H), 1.77 |
| | | (s, 3H), 1.60 (s, 3H), 1.59 (s, 3H). |
| 22.1 | 511.9 | 1H NMR (400 MHz, DMSO-d6) δ 8.52-8.46 (m, 2H), 8.43 (d, J = 2.7 |
| | | Hz, 1H), 8.36 (d, J = 2.6 Hz, 1H), 7.67 (dt, J = 10.5, 2.3 Hz, 1H), 6.58 |
| | | (d, J = 2.6 Hz, 1H), 6.31 (s, 1H), 2.62 (t, J = 7.5 Hz, 2H), 2.10 (s, 3H), |
| | | 2.02 (s, 3H), 1.87-1.77 (m, 2H), 1.46 (s, 6H). |

### BIOLOGICAL EXAMPLES

### Buffers

The following buffers were used in the assays:
25 mM Hepes, pH 7.6 10 mM MgCL2, 0.01% TritonX100, 0.01% BSA 1 mM

DTT for all assays
MgATP: catalog #B20 Fisher Scientific
DTT: catalog #P2325 Fisher Scientific
BSA: catalog #10711454001 Sigma Aldrich
Hepes: catalog #J61275.AK Thermo Fisher
MgCl2: catalog #AM9530G Thermo Fisher
Triton X100: catalog #A16046.AE Thermo Fisher
   p38α:MK2:HSP27

The p38α:MK2:HSP27 biochemical assay was used to determine the concentration of tested compounds to inhibit 50% of maximal MK2 activation by p38 (IC₅₀). In the reaction, the active p38 phosphorylates and activates MK2, and the activated MK2 then phosphorylates HSP27 peptide. The activity of MK2 was determined by the phosphorylation level of HSP27 peptide.

In a 20 μL reaction, the compounds of various concentrations (20 nL) were pre-incubated with 0.6 nM MK2 (Abcam #ab79910) and 0.06 nM active p38α (Abcam #ab271606 or in-house prepared: Avi-Tev-8HIS-p38a phosphorylated by GST-MKK6) for 15 minutes at room temperature following by the addition of 2 μMH SP27 peptide substrate ((TAMRA)Cys-KKKALSRQLSVAA) (custom synthesized, Elim Biopharmaceuticals) and 10p M ATP (Fisher Scientific #B20). After 1 h incubation at room temperature, 60 μL detection reagent (Molecular Devices #8160) was added to the reaction and incubate d for 12 h at room temperature. Homogeneous Time Resolved Fluorescence (HTRF) signal (the ratio between emission at 550 nm and emission at 570 nm upon excitation at 330 nm) was read by TECAN Spark microplate reader. The signal was normalized to vehicle control (DMSO, 0% inhibition) and 1 μM GS-703447 (100% inhibition) to generate % inhibition as a function of compound concentration and fitted in a four-parameter logistic equation to generate IC₅₀, as shown in Table 14.

p38α:eIF4E-BP1

The p38α:eIF4E-BP1 biochemical assay was used to determine the concentration of tested compounds to inhibit 50% of p38 activity (IC₅₀). The activity of p38 was determined by the phosphorylation level of eIF4E-binding protein 1 peptide.

In a 10 μL reaction, the compounds of various concentrations (10 nL) were pre-incubated with 3 nM active p38 at (Abcam #ab271606 or in-house prepared: Avi-Tev-8HIS-p38a phosphorylated by GST-MKK6) for 15 minutes at room temperature following by the addition of 120 nM ULight-eIF4E-binding protein 1 (Thr37/46) peptide (Perkin Elmer #TRF-0128M) and 100 M ATP (Fisher Scientific #B320). After 1 h incubation at room temperature, 5 μL detection reagent (50 mM EDTA, TX Lance reaction buffer (Perkin Elmer #TRFLAB100) and 2 nM Lance Ultra Europium-anti-phospho-eIF4E-binding protein 1 Thr37/46 (Perkin Elmer #TRF-0216M)) were added to the reaction and incubated for 1 h at room temperature. HTRF signal (the ratio between emission at 620 nm and emission at 665 nm upon excitation at 330 nm) was read by TECAN Spark microplate reader. The signal was normalized to vehicle control (DMSO, 0% inhibition) and 1 μM GS-703447 (100% inhibition) to generate IC₅₀ inhibition as a function of compound concentration and filled in a four-parameter logistic equation to generate IC₅₀, as shown in Table 14.

TABLE 17
Assay results

| Example Number | p38-MK2 IC₅₀ (nM) | p38-Peptide IC₅₀ (nM) |
|---|---|---|
| 1.1 | 422.0 | >10000 |
| 1.2 | 25.7 | 9471.7 |
| 1.3 | 28.1 | 921.1 |
| 1.4 | 17.8 | 1070.2 |
| 1.5 | 41.3 | 376.6 |
| 1.6 | 87.2 | ND |
| 1.7 | 44.7 | ND |
| 1.8 | 7456.9 | ND |
| 1.9 | 468.6 | >10000 |
| 1.10 | 129.6 | 1576.0 |
| 1.11 | 72.1 | >1000 |
| 1.12 | 329.6 | >10000 |
| 1.13 | >1000 | >1000 |
| 1.14 | >1000 | >1000 |
| 1.15 | 731.2 | 2263.6 |
| 1.16 | 754.2 | >10000 |
| 1.17 | 33.0 | >10000 |
| 1.18 | 569.2 | >10000 |
| 1.19 | 105.2 | 4898.3 |
| 1.20 | 721.0 | >10000 |
| 1.21 | >1000 | >10000 |
| 1.22 | >1000 | >10000 |
| 1.23 | >1000 | >10000 |
| 1.24 | >1000 | >10000 |
| 1.25 | >1000 | >10000 |
| 1.26 | >1000 | >10000 |
| 1.27 | 511.0 | >10000 |
| 1.28 | 809.7 | >10000 |
| 1.29 | >1000 | >10000 |
| 1.30 | >1000 | >10000 |
| 1.31 | >1000 | >10000 |
| 1.32 | 184.0 | >10000 |
| 1.33 | >1000 | >10000 |
| 1.34 | 24.4 | 5925.0 |
| 1.35 | >1000 | >10000 |
| 1.36 | 695.3 | >10000 |
| 1.37 | 166.4 | 5882.9 |
| 1.38 | 954.3 | >10000 |
| 1.39 | 27.1 | 815.6 |
| 1.40 | 613.8 | >10000 |
| 1.41 | 381.6 | >10000 |
| 1.42 | 107.1 | 5586.5 |
| 1.43 | 107.1 | 2353.6 |
| 1.44 | >1000 | >10000 |
| 1.45 | 44.3 | 8576.3 |
| 1.46 | 137.7 | 5676.7 |
| 1.47 | 48.4 | 3039.0 |
| 1.48 | 36.5 | 631.3 |
| 1.49 | 32.0 | 92.4 |
| 1.50 | 160.5 | 7711.8 |
| 1.51 | 501.4 | >10000 |
| 1.52 | 561.7 | 5782.7 |
| 1.53 | 23.6 | 3260.7 |
| 1.54 | >1000 | >10000 |
| 1.55 | 38.6 | 2381.3 |
| 1.56 | 127.0 | >10000 |
| 1.57 | 19.0 | 137.4 |
| 1.58 | 212.8 | >10000 |
| 1.59 | >1000 | >10000 |
| 1.60 | 760.7 | >10000 |
| 1.61 | 114.3 | >10000 |
| 1.62 | 424.1 | 4732.8 |
| 1.63 | >1000 | >10000 |
| 1.64 | 3.1 | 151.3 |
| 1.65 | 34.5 | 4437.5 |
| 2.1 | 59.9 | 3616.5 |
| 2.2 | 651.1 | >10000 |
| 2.3 | >1000 | >10000 |
| 3.1 | 1.4 | 131.2 |
| 3.2 | 1334.0 | ND |
| 3.3 | 0.4 | 100.8 |
| 3.4 | 5.2 | 195.7 |
| 3.5 | 3.8 | 196.7 |
| 3.6 | 3.3 | 372.4 |
| 3.7 | 1.6 | 73.5 |
| 3.8 | 1.4 | 42.6 |
| 3.9 | 5.0 | 809.0 |
| 3.10 | 724.6 | >10000 |
| 3.11 | 8.7 | 871.3 |
| 3.12 | 0.8 | 752.8 |
| 3.13 | 1.9 | 414.3 |
| 3.14 | 0.9 | 274.4 |
| 3.15 | 20.2 | 2147.7 |
| 3.16 | 1.0 | 205.4 |
| 3.17 | 2.0 | 615.9 |
| 3.18 | 10.5 | 4962.3 |
| 3.19 | 4.7 | 703.9 |
| 3.20 | 1.3 | 58.0 |
| 3.21 | 2.5 | 498.7 |
| 3.22 | 1.4 | 134.6 |
| 3.23 | 3.3 | 84.3 |
| 3.24 | 25.9 | 290.2 |
| 3.25 | 1.6 | 305.6 |
| 3.26 | 1.6 | 205.9 |
| 3.27 | 2.0 | 69.3 |
| 3.28 | 1.9 | 27.2 |
| 3.29 | 0.1 | 28.2 |
| 3.30 | 15.7 | 22.2 |
| 3.31 | 2.7 | 111.4 |
| 3.32 | 11.8 | 1552.3 |
| 3.33 | 3.8 | 348.9 |
| 3.34 | 4.2 | 347.0 |
| 3.35 | 0.5 | 17.7 |
| 3.36 | 1.4 | 110.1 |
| 3.37 | 1.4 | 97.3 |
| 3.38 | 3.3 | 522.4 |
| 3.39 | 5.7 | 863.0 |
| 4.1 | 2.2 | 256.9 |
| 4.2 | 1.6 | 679.7 |
| 4.3 | 14.1 | 8889.7 |
| 4.4 | 3.1 | 385.4 |
| 4.5 | 3.4 | 921.5 |
| 4.6 | 322.5 | >10000 |
| 4.7 | 11.1 | 2732.3 |
| 4.8 | 14.6 | 5183.5 |
| 4.9 | 2.0 | 44.1 |
| 4.10 | 0.7 | 37.2 |
| 4.11 | 1.3 | 390.2 |
| 4.12 | 1.6 | 130.6 |
| 4.13 | 80.9 | 9960.7 |
| 4.14 | 122.9 | >10000 |
| 4.15 | 1.4 | 56.5 |
| 4.16 | 3.6 | 812.5 |
| 4.17 | 2.8 | 543.9 |
| 4.18 | 3.1 | 192.5 |
| 4.19 | 2.5 | 484.8 |
| 4.20 | 7.2 | 280.9 |
| 4.21 | 9.4 | 912.7 |
| 4.22 | 5.1 | 159.8 |
| 4.23 | 6.7 | 1077.5 |
| 4.24 | 13.0 | 2608.2 |
| 4.25 | 0.8 | 147.6 |
| 4.26 | 27.2 | 9309.0 |
| 4.27 | 9.2 | 805.0 |
| 4.28 | 4.8 | 167.7 |
| 4.29 | 10.5 | 494.2 |
| 4.30 | 59.7 | 1547.3 |
| 4.31 | 3.3 | 376.4 |
| 4.32 | 171.0 | >10000 |
| 4.33 | 2.6 | 297.6 |
| 4.34 | 8.4 | 134.8 |
| 4.35 | >1000 | >10000 |
| 4.36 | 1.6 | 71.4 |
| 4.37 | 7.3 | 6396.3 |
| 4.38 | 24.9 | 3804.1 |
| 4.39 | 8.9 | 297.0 |
| 4.40 | 20.4 | 479.1 |
| 4.41 | 26.0 | 504.8 |
| 4.42 | 66.1 | 1398.5 |
| 4.43 | 4.0 | 191.0 |
| 4.44 | 1.6 | 52.6 |
| 4.45 | 3.4 | 118.4 |
| 4.46 | 10.5 | 142.5 |
| 4.47 | 41.9 | 713.3 |
| 4.48 | 26.0 | 272.6 |
| 4.49 | 7.5 | 1648.2 |
| 4.50 | 3.9 | 1008.2 |
| 4.51 | 3.7 | 100.4 |
| 4.52 | 21.1 | 4153.3 |
| 4.53 | 16.9 | 2822.5 |
| 4.54 | 1.4 | 454.4 |
| 4.55 | 8.1 | 1436.4 |
| 4.56 | 28.4 | 2234.9 |
| 4.57 | 13.0 | 1065.8 |
| 4.58 | 13.4 | 1021.8 |
| 4.59 | 23.1 | 2764.4 |
| 4.60 | 2.2 | 338.4 |
| 4.61 | 1.0 | 160.6 |
| 4.62 | 2.3 | 273.8 |
| 4.63 | 1.7 | 274.9 |
| 4.64 | 7.6 | 906.4 |
| 4.65 | 3.8 | 568.1 |
| 4.66 | 2.3 | 172.8 |
| 4.67 | 24.1 | 3562.8 |
| 4.68 | 4.4 | 692.8 |
| 4.69 | 51.0 | 4077.7 |
| 4.70 | 14.6 | 2736.3 |
| 4.71 | 3.2 | 342.7 |
| 4.72 | 3.6 | 233.6 |
| 4.73 | 12.8 | 1926.5 |
| 4.74 | 6.2 | 1525.9 |
| 4.75 | 3.6 | 619.0 |
| 4.76 | 20.3 | 3766.0 |
| 4.77 | 2.9 | 561.5 |
| 4.78 | 3.7 | 204.5 |
| 4.79 | 59.4 | 3938.6 |
| 4.80 | 34.7 | 7548.4 |
| 4.81 | 50.2 | 4879.6 |
| 4.82 | 2.4 | 445.6 |
| 4.83 | 2.8 | 821.7 |
| 4.84 | 12.6 | 2173.2 |
| 4.85 | 13.4 | 1745.3 |
| 4.86 | 79.4 | 9164.6 |
| 4.87 | 76.7 | 7134.2 |
| 4.88 | 49.0 | 3581.0 |
| 4.89 | 13.1 | 831.2 |
| 4.90 | 5.9 | 275.1 |
| 4.91 | 6.1 | 184.8 |
| 4.92 | 37.6 | 1548.8 |
| 4.93 | 6.2 | 418.0 |
| 4.94 | 32.9 | 3055.8 |
| 4.95 | 19.0 | 2694.4 |
| 4.96 | 17.4 | 584.2 |
| 4.97 | 37.6 | 4857.5 |
| 4.98 | 4.8 | 762.7 |
| 4.99 | 6.9 | 584.2 |
| 4.100 | 1.4 | 34.4 |
| 4.101 | 0.3 | 6.3 |
| 4.102 | 33.6 | 1475.8 |
| 4.103 | 12.9 | 561.4 |
| 4.104 | 7.9 | 303.0 |
| 4.105 | 3.4 | 917.1 |
| 4.106 | 3.9 | 567.9 |
| 4.107 | 2.5 | 61.3 |
| 4.108 | 1.4 | 82.2 |
| 4.109 | 70.0 | 1784.8 |
| 4.110 | 3.8 | 136.8 |
| 4.111 | 4.7 | 726.1 |
| 4.112 | 3.2 | 418.6 |
| 4.113 | 1.5 | 250.8 |
| 4.114 | 2.6 | 242.0 |
| 4.115 | 12.9 | 956.0 |
| 4.116 | 0.9 | 97.3 |
| 4.117 | 26.2 | 530.1 |
| 4.118 | 3.1 | 1287.1 |
| 4.119 | 11.0 | 927.1 |
| 4.120 | 0.7 | 159.1 |
| 4.121 | 76.4 | >10000 |
| 4.122 | 19.1 | 2704.5 |
| 4.123 | 23.8 | 1053.3 |
| 4.124 | 19.8 | 2517.6 |
| 4.125 | 44.0 | 4197.5 |
| 4.126 | 1.4 | 122.8 |
| 4.127 | 71.3 | 8247.1 |
| 4.128 | 0.3 | 31.4 |
| 4.129 | 30.7 | 2128.0 |
| 4.130 | 5.4 | 238.8 |
| 4.131 | 1.6 | 141.4 |
| 4.132 | 2.5 | 320.5 |
| 4.133 | 4.7 | 249.8 |
| 4.134 | 1.4 | 96.9 |
| 4.135 | 3.6 | 261.6 |
| 4.136 | 2.9 | 418.1 |
| 4.137 | 0.8 | 357.4 |
| 4.138 | 89.4 | >10000 |
| 4.139 | 36.9 | 3889.0 |
| 4.140 | 1.5 | 266.0 |
| 4.141 | 6.1 | 234.1 |
| 4.142 | 7.8 | 198.8 |
| 4.143 | 6.4 | 118.6 |
| 4.144 | 11.7 | 657.0 |
| 4.145 | 2.4 | 123.8 |
| 4.146 | 27.2 | 530.5 |
| 4.147 | 14.2 | 367.8 |
| 4.148 | 3.2 | 534.0 |
| 4.149 | 3.3 | 681.0 |
| 4.150 | 0.9 | 101.9 |
| 4.151 | 9.6 | 494.9 |
| 4.152 | 11.8 | 974.3 |
| 4.153 | 3.3 | 50.8 |
| 4.154 | 4.7 | 226.5 |
| 4.155 | 2.7 | 179.4 |
| 4.156 | 33.9 | 1275.2 |
| 4.157 | 10.6 | 227.7 |
| 4.158 | 3.6 | 186.9 |
| 4.159 | 7.4 | 92.8 |
| 4.160 | 4.0 | 296.8 |
| 4.161 | 3.5 | 232.6 |
| 4.162 | 2.6 | 105.2 |
| 4.163 | 10.9 | 194.1 |
| 4.164 | 3.1 | 113.2 |
| 4.165 | 2.3 | 66.5 |
| 4.166 | 39.9 | 8389.8 |
| 4.167 | 3.0 | 226.6 |
| 4.168 | 7.7 | 573.2 |
| 4.169 | 3.1 | 84.7 |
| 4.170 | 1.8 | 115.9 |
| 4.171 | 0.8 | 59.6 |
| 4.172 | 11.0 | 386.4 |
| 4.173 | 12.4 | 622.2 |
| 4.174 | 6.7 | 887.0 |
| 4.175 | 6.7 | 536.1 |
| 4.176 | 5.1 | 178.4 |
| 4.177 | 5.1 | 578.4 |
| 4.178 | 4.0 | 247.5 |
| 4.179 | 1.6 | 324.2 |
| 4.180 | 6.2 | 528.0 |
| 4.181 | 1.9 | 270.5 |
| 4.182 | 3.4 | 791.0 |
| 4.183 | 4.9 | 1648.8 |
| 4.184 | 3.3 | 867.8 |
| 4.185 | 1.9 | 94.1 |
| 4.186 | 15.7 | 1158.9 |
| 4.187 | 8.3 | 364.9 |
| 4.188 | 1.4 | 121.8 |
| 4.189 | 12.4 | 757.8 |
| 4.190 | 8.4 | 75.7 |
| 4.191 | 10.9 | 405.4 |
| 4.192 | 9.2 | 117.9 |
| 4.193 | 0.6 | 34.6 |
| 4.194 | 1.0 | 47.4 |
| 4.195 | 1.8 | 70.3 |
| 4.196 | 4.7 | 46.7 |
| 4.197 | 1.6 | 121.2 |
| 4.198 | 1.4 | 85.6 |
| 4.199 | 10.1 | 893.5 |
| 4.200 | 5.2 | 186.5 |
| 5.1 | 10.1 | 70.5 |
| 5.2 | 6.7 | 133.8 |
| 5.3 | 21.9 | 1618.4 |
| 5.4 | 16.1 | 2743.6 |
| 5.5 | 9.3 | 2189.1 |
| 5.6 | 4.5 | 3605.9 |
| 5.7 | 16.5 | 1093.7 |
| 5.8 | 11.7 | 675.8 |
| 5.9 | 2.9 | 103.4 |
| 5.10 | 3.4 | 299.2 |
| 5.11 | 6.4 | 354.1 |
| 5.12 | 1.2 | 102.5 |
| 5.13 | 1.0 | 67.6 |
| 5.14 | 1.7 | 76.2 |
| 5.15 | 0.7 | 44.1 |
| 5.16 | 1.7 | 518.4 |
| 5.17 | 3.3 | 558.9 |
| 5.18 | 1.3 | 42.2 |
| 5.19 | 0.4 | 36.7 |
| 5.20 | 3.8 | 87.5 |
| 5.21 | 30.9 | 176.5 |
| 5.22 | 177.9 | 233.7 |
| 5.23 | 835.4 | 479.2 |
| 5.24 | 0.8 | 125.3 |
| 5.25 | 0.7 | 146.1 |
| 5.26 | 951.5 | 2320.5 |
| 5.27 | 0.7 | 96.5 |
| 5.28 | 1.1 | 133.3 |
| 5.29 | 0.4 | 96.2 |
| 5.30 | 1.8 | 95.3 |
| 5.31 | 1.1 | 53.2 |
| 5.32 | 0.8 | 46.6 |
| 5.33 | 1.2 | 118.1 |
| 5.34 | 1.7 | 123.4 |
| 5.35 | 0.4 | 29.6 |
| 5.36 | 0.8 | 47.3 |
| 5.37 | 0.9 | 38.3 |
| 5.38 | 0.2 | 17.9 |
| 5.39 | 0.4 | 28.5 |
| 5.40 | 0.2 | 17.7 |
| 5.41 | 0.9 | 112.8 |
| 5.42 | 0.5 | 85.7 |
| 5.43 | 4.2 | 163.4 |
| 5.44 | 0.4 | 7.9 |
| 5.45 | 0.3 | 11.8 |
| 5.46 | 0.9 | 43.7 |
| 5.47 | 0.4 | 28.4 |
| 5.48 | 2.4 | 130.2 |
| 5.49 | 0.6 | 78.8 |
| 5.50 | 0.4 | 32.4 |
| 5.51 | 1.1 | 109.5 |
| 5.52 | 2.7 | 76.1 |
| 5.53 | 0.9 | 29.7 |
| 5.54 | 0.8 | 32.6 |
| 5.55 | 0.3 | 19.8 |
| 5.56 | 0.2 | 16.3 |
| 5.57 | 0.5 | 40.9 |
| 5.58 | 0.6 | 60.8 |
| 5.59 | 0.1 | 24.9 |
| 5.60 | 0.7 | 23.4 |
| 5.61 | 0.3 | 14.1 |
| 5.62 | 0.4 | 9.8 |
| 5.63 | 0.8 | 20.0 |
| 6.1 | 250.5 | 1591.2 |
| 6.2 | 140.1 | 1172.1 |
| 6.3 | 7.3 | 206.3 |
| 6.4 | 4.6 | 3033.0 |
| 6.5 | 193.3 | >10000 |
| 6.6 | 127.8 | 2470.0 |
| 6.7 | 11.3 | 192.2 |
| 6.8 | >1000 | 3527.8 |
| 6.9 | 410.3 | >10000 |
| 6.10 | 107.2 | 6041.6 |
| 6.11 | 5.4 | 214.7 |
| 6.12 | 3.3 | 33.5 |
| 6.13 | 9.1 | 779.4 |
| 6.14 | 2.8 | 1020.5 |
| 6.15 | 1.4 | 55.0 |
| 6.16 | >1000 | >10000 |
| 6.17 | 379.1 | 903.5 |
| 6.18 | >1000 | ND |
| 6.19 | 207.1 | 2033.6 |
| 6.20 | 0.7 | 41.3 |
| 6.21 | 27.1 | 637.9 |
| 6.22 | 15.3 | 241.9 |
| 6.23 | 79.0 | 991.5 |
| 6.24 | 19.6 | 320.8 |
| 6.25 | 4.4 | 546.6 |
| 6.26 | >1000 | >10000 |
| 6.27 | 2.6 | 528.8 |
| 6.28 | 2.3 | 360.7 |
| 6.29 | 2.1 | 119.6 |
| 6.30 | 23.1 | 428.2 |
| 6.31 | 4.9 | 120.3 |
| 6.32 | 0.8 | 65.7 |
| 6.33 | 0.3 | 35.7 |
| 6.34 | 0.4 | 18.9 |
| 6.35 | 0.4 | 11.0 |
| 6.36 | 0.8 | 53.3 |
| 6.37 | 1.3 | 32.1 |
| 6.38 | 0.9 | 17.8 |
| 6.39 | 0.5 | 79.4 |
| 6.40 | 1.0 | 29.3 |
| 6.41 | 0.7 | 68.1 |
| 6.42 | 0.6 | 38.6 |
| 6.43 | 1.7 | 119.7 |
| 6.44 | 9.4 | 162.6 |
| 6.45 | 1.9 | 179.7 |
| 6.46 | 1.1 | 127.3 |
| 6.47 | 0.2 | 8.5 |
| 6.48 | 1.4 | 109.3 |
| 6.49 | 21.4 | 916.3 |
| 6.50 | 8.0 | 438.8 |
| 6.51 | 13.0 | 1354.7 |
| 6.52 | 5.6 | 775.5 |
| 6.53 | 27.5 | 248.2 |
| 6.54 | 1.8 | 33.3 |
| 6.55 | 8.0 | 356.4 |
| 6.56 | 3.2 | 98.7 |
| 6.57 | 1.5 | 47.1 |
| 6.58 | 2.8 | 61.2 |
| 6.59 | 1.8 | 53.6 |
| 6.60 | 2.2 | 55.3 |
| 6.61 | 2.4 | 69.3 |
| 6.62 | 4.5 | 61.7 |
| 6.63 | 6.2 | 286.0 |
| 6.64 | 0.4 | 9.2 |
| 6.65 | 0.5 | 34.8 |
| 6.66 | 0.6 | 33.4 |
| 6.67 | 0.3 | 10.0 |
| 6.68 | 2.7 | 78.3 |
| 6.69 | 1.2 | 66.4 |
| 6.70 | 0.5 | 40.4 |
| 6.71 | 0.6 | 54.4 |
| 6.72 | 0.9 | 53.8 |
| 6.73 | 8.9 | 480.5 |
| 6.74 | 1.3 | 40.2 |
| 6.75 | 12.0 | 412.4 |
| 6.76 | 23.4 | 540.4 |
| 6.77 | 23.7 | 826.3 |
| 6.78 | 2.4 | 354.0 |
| 6.79 | 0.9 | 173.2 |
| 6.80 | 1.2 | 130.8 |
| 6.81 | 1.6 | 133.4 |
| 6.82 | 1.0 | 66.4 |
| 6.83 | 6.7 | 734.9 |
| 6.84 | 0.9 | 56.2 |
| 6.85 | 75.8 | 4006.8 |
| 6.86 | 5.2 | 1821.5 |
| 6.87 | 4.2 | 48.8 |
| 7.1 | 3.9 | 77.6 |
| 7.2 | 5.8 | 175.8 |
| 7.3 | 24.4 | 167.1 |
| 7.4 | 45.0 | 348.9 |
| 8.1 | 22.6 | 124.4 |
| 8.2 | 1.2 | 51.7 |
| 9.1 | 370.8 | >10000 |
| 9.2 | >1000 | >10000 |
| 9.3 | >1000 | >10000 |
| 10.1 | 12.8 | 201.2 |
| 10.2 | 9.6 | 140.0 |
| 10.3 | 1.6 | 99.5 |
| 10.4 | 2.5 | 224.6 |
| 10.5 | 1.0 | 27.2 |
| 10.6 | 1.5 | 45.8 |
| 10.7 | 1.0 | 26.8 |
| 10.8 | 1.0 | 39.6 |
| 10.9 | 1.6 | 54.8 |
| 10.10 | 0.7 | 30.5 |
| 10.11 | 0.4 | 16.7 |
| 11.1 | 2.8 | 93.3 |
| 12.1 | 38.2 | >10000 |
| 13.1 | 3.6 | 108.0 |
| 14.1 | 2.8 | 133.9 |
| 14.2 | 1.6 | 75.0 |
| 14.3 | 0.9 | 126.1 |
| 15.1 | 2.2 | 212.6 |
| 16.1 | 1.7 | 3.5 |
| 17.1 | 0.7 | 24.0 |
| 17.2 | 0.8 | 130.3 |
| 17.3 | 1.0 | 115.1 |
| 17.4 | 4.3 | 818.5 |
| 17.5 | 3.1 | 486.8 |
| 18.1 | 0.8 | 51.2 |
| 18.2 | 5.6 | 581.0 |
| 18.3 | 1.3 | 107.1 |
| 18.4 | 11.1 | 468.4 |
| 18.5 | 4.7 | 376.9 |
| 18.6 | 3.7 | 513.7 |
| 18.7 | 5.7 | 473.3 |
| 18.8 | 9.8 | 1144.9 |
| 18.9 | 3.2 | 100.8 |
| 19.1 | 5.8 | 674.5 |
| 19.2 | 1.0 | 70.2 |
| 19.3 | 3.6 | 688.8 |
| 19.4 | 7.4 | 788.8 |
| 19.5 | 8.3 | 879.6 |
| 19.6 | 3.7 | 536.6 |
| 20.1 | 3.4 | 1656.4 |
| 20.2 | 5.8 | 897.3 |
| 20.3 | 10.8 | 3110.0 |
| 20.4 | 11.2 | 1071.7 |
| 21.1 | 1.5 | 137.9 |
| 22.1 | 2.2 | 149.7 |

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter and is not intended to limit the appended claims to the specific embodiments illustrated.

1. A compound of Formula I: 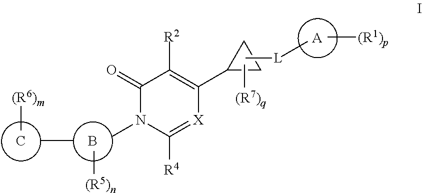
or a pharmaceutically acceptable salt thereof, wherein
ring \textcircled{A} is C₆₋₁₀ aryl, 5- to 10-membered heteroaryl, C₃₋₁₀ cycloalkyl, or 4- to 10-membered heterocyclyl;
each R^{1a} is independently halogen, —CD₃, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, —N(R^{1a})(R^{1a}), —CN, —OR^{1a}, —C(O)OR^{1a}, —C(O)R^{1a}, —C(O)N(R^{1a})(R^{1a}), —N(R^{1a})C(O)—R^{1a}, —N(R^{1a})C(O)O—R^{1a}, —N(R^{1a})C(O)N(R^{1a})(R^{1a}), —N═S(O)(R^{1a})(R^{1a}), —N(R^{1a})S(O)₂(R^{1a}), —N(R^{1a})S(O)₂N(R^{1a})(R^{1a}), —N(R^{1a})S(O)₂O(R^{1a}), —OC(O)R^{1a}, —OC(O)OR^{1a}, —OC(O)N(R^{1a})(R^{1a}), —SR^{1a}, S(O)R^{1a}, —SF₅, —S(O)(NR^{1a})R^{1a}, —S(NR^{1a})(NR^{1a})R^{1a}, —S(O)(NR^{1a})N(R^{1a})(R^{1a}), —S(O)(N—CN)R^{1a}, —S(O)₂R^{1a}, —S(O)₂N(R^{1a})(R^{1a}), —C(O)N(R^{1a})S(O)₂R^{1a}, —S(O)₂N(R^{1a})C(O)R^{1a}, or —P(O)(R^{1a})₂, or two R¹ groups taken together with two adjacent atoms of ring \textcircled{A} to which they are attached form a C₃₋₁₀ cycloalkyl or 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g};
each R^{1a} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, two R^{1a} groups taken together with the nitrogen atom to which they are attached form a 4- to 10-membered heterocyclyl, wherein each alkyl, alkenyl, alkynyl, cycloalkyl, heterocyclyl, aryl, and heteroaryl is optionally substituted with one to three R¹ⁱ;
each R^{1g} is independently —CN, C₁₋₆ alkyl, C₂₋₆ alkynyl, C₃₋₁₀ cycloalkyl, 4- to 10-membered heterocyclyl, C₆₋₁₀ aryl, 5- to 10-membered heteroaryl, —OR^{1h}, —N(R^{1h})(R^{1h}), —C(O)N(R^{1h})(R^{1h}), —N(R^{1h})C(O)—R^{1h}, —N(R^{1h})C(O)O—R^{1h}, —N(R^{1h})C(O)N(R^{1h})(R^{1h}), —OC(O)N(R^{1h})(R^{1h}), —N(R^{1a})S(O)₂(R^{1a}), —S(O)₂R^{1h}, or —S(O)₂N(R^{1a})(R^{1a}), wherein each alkyl, alkynyl, cycloalkyl, heterocyclyl, aryl, and heteroaryl is optionally substituted with one to three R¹ⁱ;
each R^{1h} is independently H, C₁₋₆ alkyl, C₂₋₆ alkynyl, C₃₋₁₀ cycloalkyl, 4- to 10-membered heterocyclyl, C₆₋₁₀ aryl, or 5- to 10-membered heteroaryl, or two Rh groups taken together with the nitrogen atom to which they are attached form a 4- to 10-membered heterocyclyl, wherein each alkyl, alkynyl, cycloalkyl, heterocyclyl, aryl and heteroaryl may be further substituted with one to three R¹ⁱ;
each R¹ⁱ is independently halogen, —OH, —NH₂, —CN, C₁₋₆ alkyl, C₁₋₆ haloalkyl, C₁₋₆ hydroxyalkyl, C₃₋₆ cycloalkyl, C₃₋₆ halocycloalkyl, C₁₋₆ alkoxy, C₁₋₆ haloalkoxy, or —O(C₃₋₆ cycloalkyl);
L is a bond, —O—, —NR^{La}—, —C(O)NR^{La}—, —C(O)—, or C₁₋₃ alkylene, wherein the alkylene is optionally substituted with one to three R^{Lb};
each R^{La} is independently H, C₁₋₆ alkyl, C₁₋₆ haloalkyl, or C₃₋₁₀ cycloalkyl;
each R^{Lb} is independently halogen, —OH, —OCH₃, —NH₂, or —CN;
each R^{Y} is independently halogen, —OH, —NH₂, —CN, C₁₋₆ alkyl, C₁₋₆ haloalkyl, C₁₋₆ hydroxyalkyl, C₁₋₆ alkoxy, or C₁₋₆ haloalkoxy;
or R^{Y} and R¹ join together to form a C₃₋₆ cycloalkyl or 4- to 6-membered heterocyclyl, wherein each cycloalkyl and heterocyclyl is optionally substituted with one to three halogen;
X is CR³ or N;
R² is H, halogen, C₁₋₆ alkyl, or C₃₋₆ cycloalkyl, wherein each alkyl and cycloalkyl is optionally substituted with one to three halogen;
R³ is H, halogen, —CN, C₁₋₆ alkyl, C₁₋₆ haloalkyl, C₁₋₆ hydroxyalkyl, C₁₋₆ alkoxy, C₁₋₆ haloalkoxy, or —O(C₃₋₆ cycloalkyl);
R⁴ is H, —CN, C₁₋₆ alkyl, C₂₋₆ alkynyl, or C₃₋₁₀ cycloalkyl, wherein each alkyl, alkynyl, and cycloalkyl is optionally substituted with one to three R^{4a1};
each R^{4a1} is independently halogen, —CN, —OR^{4a2}, or —NR^{4a2}R^{4a2};
each R^{4a2} is independently H, C₁₋₆ alkyl, C₁₋₆ haloalkyl, C₃₋₆ cycloalkyl, —C(O)C₁₋₆ alkyl, —C(O)O(C₁₋₆ alkyl), or —C(O)N(R^{4a3})₂;
each R^{4a3} is independently C₁₋₆ alkyl;
ring \textcircled{B} is C₆₋₁₀ aryl or 5- to 10-membered heteroaryl;
each R⁵ is independently halogen, —CN, C₁₋₆ alkyl, C₂₋₆ alkenyl, C₂₋₆ alkynyl, C₃₋₁₀ cycloalkyl, —OR^{5a1}, —N(R^{5a1})₂, —N(R^{5a1})C(O)(R^{5a1}), —C(O)N(R^{5a1})₂, —N(R^{5a1})S(O)₂(R^{5a1}), —SO₂N(R^{5a1})₂, —SO₂(R^{5a1}), or —SR^{5a1}, wherein each alkyl, alkenyl, alkynyl and cycloalkyl is optionally substituted with one to three R^{5b1};
each R^{5a1} is independently H, C₁₋₆ alkyl, C₃₋₁₀ cycloalkyl, or 4- to 10-membered heterocyclyl, wherein each alkyl, cycloalkyl, and heterocyclyl is optionally substituted with one to three R^{5b3};
each R^{5b1} is independently halogen, —CN, C₁₋₆ alkyl, C₂₋₆ alkynyl, C₁₋₆ haloalkyl, C₃₋₁₀ cycloalkyl, 4- to 10-membered heterocyclyl, —OR^{5b2}, or —N(R^{5b2})₂, wherein each alkyl, alkynyl, haloalkyl, cycloalkyl, and heterocycle is optionally substituted with one to three R^{5b3};
each R^{5b2} is independently H, C₁₋₆ alkyl, or C₃₋₁₀ cycloalkyl, wherein each alkyl and cycloalkyl is optionally substituted with one or two oxo and is further optionally substituted with one to three R^{5b3};
each R^{5b3} is independently halogen, —CN, —OH, —NH₂, C₁₋₆ alkyl, C₁₋₆ haloalkyl, C₁₋₆ hydroxyalkyl, C₁₋₆ alkoxy, C₁₋₆ haloalkoxy, C₃₋₆ cycloalkyl, C₃₋₆ halocycloalkyl, or —O(C₃₋₆ cycloalkyl);
ring \textcircled{C} is C₆₋₁₀ aryl, 5- to 10-membered heteroaryl, C₃₋₈ cycloalkyl, or 4- to 10-membered heterocyclyl;
each R⁶ is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6a1}, —N(R^{6a1})₂, —N(R^{6a1})₃⁺, —C(O)R^{6a1}, —C(O)OR^{6a1}, —C(O)N(R^{6a1})₂, —N(R^{6a1})C(O)R^{6a1}, —N(R^{6a1})C(O)OR^{6a1}, N(R^{6a1})C(O)N(R^{6a1})₂, —N═S(O)(R^{6a1})₂, —N═S(O)N(R^{6a1})₂R^{6a1}, —N(R^{6a1})S(O)₂(R^{6a1}), —N(R^{6a1})S(O)₂N(R^{6a1})₂, —N(R^{6a1})S(O)₂O(R^{6a1}), —OC(O)R^{6a1}, —OC(O)OR^{6a1}, —OC(O)N(R^{6a1})₂, —Si(R^{6a1})₃, —SR^{6a1}, —S(O)R^{6a1}, —SF₅, —S(O)(NR^{6a1})R^{6a1}, —S(NR^{6a1})₂R^{6a1}, —S(O)(NR^{6a1})N(R^{6a1})₂, —S(O)(N—CN)R^{6a1}, —S(O)₂R^{6a1}, —S(O)₂N(R^{6a1})₂, —C(O)N(R^{6a1})S(O)₂R^{6a1}, —S(O)₂N(R^{6a1})C(O)R^{6a1}, or —P(O)(R^{6a1})₂, or two R⁶ groups taken together with two adjacent atoms of ring \textcircled{C} to which they are attached form a C₃₋₁₀ cycloalkyl or a 4- to 10-membered heterocyclyl, wherein each alkyl, alkenyl, alkynyl, cycloalkyl, heterocyclyl, aryl, and heteroaryl is optionally substituted with one to three halogen and is further optionally substituted with one to three R^{6b1};
each R^{6a1} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, or two R^{6a1} groups taken together with the atom or atoms to which they are attached form a 4- to 10-membered heterocyclyl, wherein each alkyl, alkenyl, cycloalkyl, heterocyclyl, aryl, and heteroaryl is optionally substituted with one to three R^{6b1};
each R^{6b1} is independently oxo, halogen, —NO₂, —N₃, —CN, C₁₋₆ alkyl, C₂₋₆ alkenyl, C₂₋₆ alkynyl, C₃₋₁₀ cycloalkyl, 4- to 10-membered heterocyclyl, C₆₋₁₀ aryl, 5- to 10-membered heteroaryl, —OR^{6b2}, —C(O)—R^{6b2}, —C(O)O—R^{6b2}, —C(O)N(R^{6b2})₂, —N(R^{6b2})₂, —N(R^{6b2})₃⁺, —N(R^{6b2})C(O)R^{6b2}, —N(R^{6b2})C(O)OR^{6b2}, —N(R^{6b2})C(O)N(R^{6b2})₂, —N═S(O)(R^{6b2})₂, —N(R^{6b2})S(O)₂(R^{6b2}), —N(R^{6b2})S(O)₂N(R^{6b2})₂, —N(R^{6b2})S(O)₂O(R^{6b2}), —OC(O)R^{6b2}, —OC(O)OR^{6b2}, —OC(O)N(R^{6b2})₂, —Si(R^{6b2})₃, —SR^{6b2}, —S(O)R^{6b2}, —SF₅, —S(O)(NR^{6b2})R^{6b2}, —S(NR^{6b2})₂R^{6b2}, —S(O)(NR^{6b2})N(R^{6b2})₂, —S(O)(N—CN)R^{6b2}, —S(O)₂R^{6b2}, —S(O)₂N(R^{6b2})₂, —C(O)N(R^{6b2})S(O)₂R^{6b2}, —S(O)₂N(R^{6b2})C(O)R^{6b2}, or —P(O)(R^{6b2})₂, wherein each alkyl, alkenyl, alkynyl, cycloalkyl, heterocyclyl, aryl, and heteroaryl is optionally substituted with one to three R^{6c1};
each R^{6b2} is independently H, C₁₋₆ alkyl, C₃₋₆ alkenyl, C₃₋₆ alkynyl, C₃₋₁₀ cycloalkyl, 4- to 10-membered heterocyclyl, C₆₋₁₀ aryl, or 5- to 10-membered heteroaryl, wherein each alkyl, alkenyl, cycloalkyl, heterocyclyl, aryl, and heteroaryl is optionally substituted with one to three R^{6c2};
each R^{6c1} and R^{6c2} is independently oxo, halogen, —NO₂, —N₃, —CN, —OH, R^{6d1}, —O(R^{6d1}), —OC(O)(R^{6d1}), —NH₂, —NH(R^{6d1}), —N(R^{6d1})₂, —C(O)(R^{6d1}), —C(O)O(R^{6d1}), —C(O)NH₂, —C(O)NH(R^{6d1}), —C(O)N(R^{6d1})₂, —NHC(O)(R^{6d1}), —NHC(O)O(R^{6d1}), —NHC(O)NH(R^{6d1}), —SH, —S(R^{6d1}), —NHS(O)(R^{6d1}), —N(R^{6d1})(S(O)(R^{6d1}), —S(O)N(R^{6d1})₂, —S(O)(R^{6d1}), —S(O)(NH)(R^{6d1}), —S(O)₂(R^{6d1}), —S(O)₂NH(R^{6d1}), or —S(O)₂N(R^{6d1})₂;
each R^{6d1} is independently C₁₋₆ alkyl, C₃₋₁₀ cycloalkyl, 4- to 10-membered heterocyclyl, C₆₋₁₀ aryl, or 5- to 10-membered heteroaryl, wherein each alkyl, alkenyl, alkynyl, cycloalkyl, heterocyclyl aryl, and heteroaryl is optionally substituted with one to three R^{6e1};
each R^{6e1} is independently oxo, halogen, —NO₂, —N₃, —CN, —OH, —NH₂, methyl, or halomethyl;
wherein each heterocyclyl and heteroaryl independently has one to four ring heteroatoms each independently selected from N, O, and S; and
m is 0, 1, 2 or 3;
n is 0, 1, 2 or 3;
p is 0, 1, 2 or 3; and
q is 0, 1, 2 or 3.

2. The compound of claim 1, or a pharmaceutically acceptable salt thereof, wherein \textcircled{A} is C₆₋₁₀ aryl or 5- to 10-membered heteroaryl.

3. The compound of claim 2, or a pharmaceutically acceptable salt thereof, wherein ring \textcircled{A} is 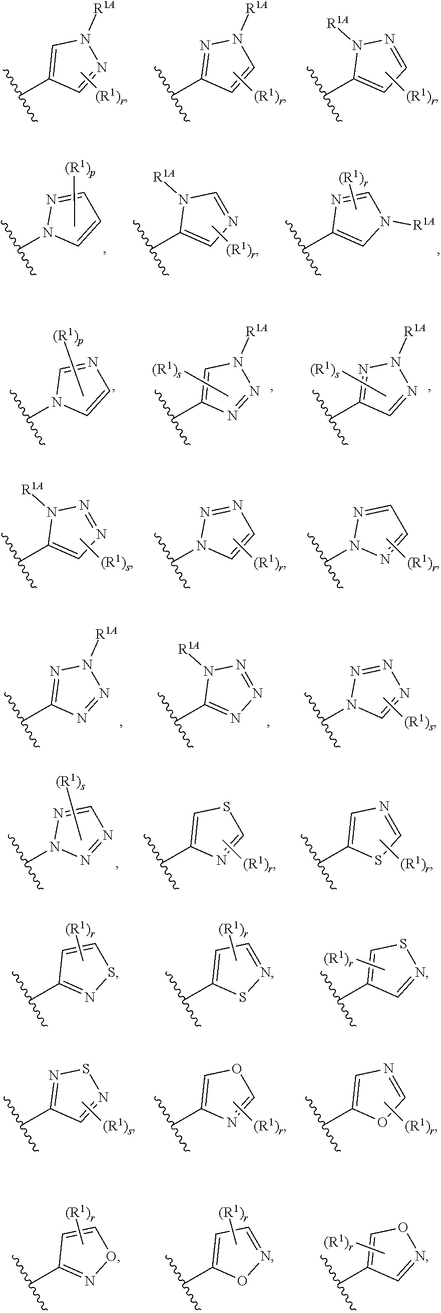 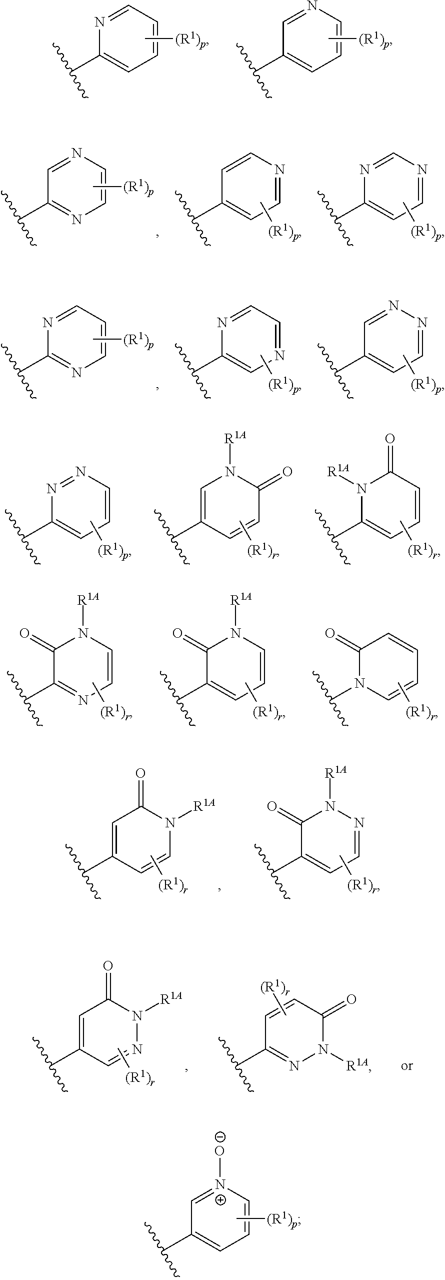
R^{1A} is H or R¹;
r is 0, 1, or 2; and
s is 0 or 1.

4. The compound of claim 1, or a pharmaceutically acceptable salt thereof, wherein ring \textcircled{A} is

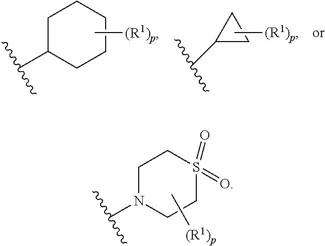

5. The compound of claim 1, or a pharmaceutically acceptable salt thereof, wherein each R¹ is independently halogen, —CD₃, C₁₋₆ alkyl, C₃₋₁₀ cycloalkyl, 4- to 6-membered heterocyclyl, —N(R^{1a})(R^{1a}), —CN, —OR^{1a}, —C(O)OR^{1a}, —C(O)N(R^{1a})(R^{1a}), —N(R^{1a})C(O)—R^{1a}, —S(O)₂N(R^{1a})(R^{1a}), —S(NR^{1a})(O)(R^{1a}), —N═S(O)(R^{1a})(R^{1a}), or two R¹ groups taken together with two adjacent atoms of ring \textcircled{A} to which they are attached form a 4- to 10-membered heterocyclyl, wherein each alkyl, alkynyl, cycloalkyl, heterocyclyl, aryl, and heteroaryl is optionally substituted with one to three halogen and is further optionally substituted with one to three R^{1g}.

6. The compound of claim 5, wherein R¹ or R^{1A} or both R¹ and R^{1A} are —CD₃.

7. The compound of claim 1, or a pharmaceutically acceptable salt thereof, wherein ring \textcircled{B} is C₆₋₁₀ aryl.

8. The compound of claim 1, or a pharmaceutically acceptable salt thereof, wherein ring \textcircled{B} is 5- to 10-membered heteroaryl.

9. The compound of claim 1, or a pharmaceutically acceptable salt thereof, wherein ring \textcircled{C} is C₆₋₁₀ aryl.

10. The compound of claim 1, or a pharmaceutically acceptable salt thereof, wherein ring \textcircled{C} is 5- to 10-membered heteroaryl.

11. The compound of claim 10, or a pharmaceutically acceptable salt thereof, wherein ring \textcircled{C} is 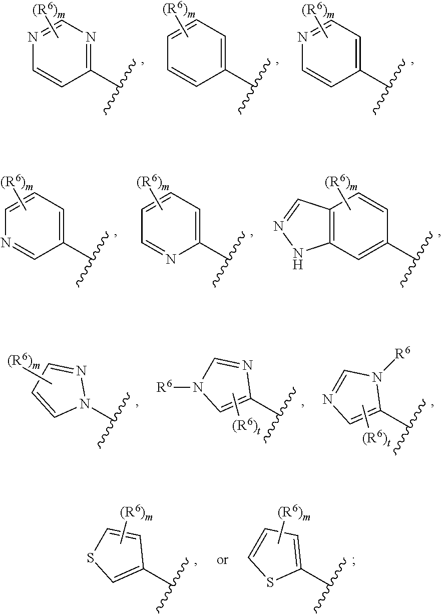
wherein t is 0, 1, or 2.

12. The compound of claim 1, or a pharmaceutically acceptable salt thereof, wherein m is 1, 2, or 3.

13. A compound, or a pharmaceutically acceptable salt thereof, selected from Table I or a pharmaceutically acceptable salt thereof.

14. A pharmaceutical composition comprising the compound of claim 1, or a pharmaceutically acceptable salt thereof, and a pharmaceutically acceptable excipient or carrier.

15. The pharmaceutical composition of claim 14, further comprising one or more additional therapeutic agents, or a pharmaceutically acceptable salt thereof.

16. (canceled)

17. (canceled)

18. (canceled)

19. A method of treating an MK2-mediated disease or disorder in a subject in need thereof, comprising administering to the subject a therapeutically effective amount of the compound of claim 1, or a pharmaceutically acceptable salt thereof.

20. (canceled)

21. (canceled)

22. (canceled)

23. (canceled)

24. The method of claim 19, further comprising administering a therapeutically effective amount of one or more additional therapeutic agents, or a pharmaceutically acceptable salt thereof.

25. The method of claim 24, wherein the one or more additional therapeutic agents is selected from the group consisting of veltuzumab, PF-06835375, eculizumab, milatuzumab, SM-06, SM-03, BT-063, QX-006-N, BOS-161721, AK-101, TNX-1500, theralizumab, daxdilimab, TAK-079, felzartamab, itolizumab, anifrolumab, iscalimab, dapirolizumab pegol, lanalumab, LY-3361237, JNJ-55920839, UBP-1213, DS-7011, PFI-102, BIIB-059, obexelimab, talacotuzumab, vobarilizumab, TE-2324, PRV-3279, chloroquine, hydroxychloroquine, hydroxychloroquine sulfate, COV-08-0064; GNKS-356, AVO-101, rozibafusp alfa, VRN-02, annexuzlimab, ALPN-101, bendamustine hydrochloride, BMS-986256, NKTR-35, atacicept, telitacicept, BMS-986256, M-5049, KZR-616, KPG-818, verdinexor, ALPN-303, valziflocept, LA-1, cenerimod, prednisone, corticotropin, deucravacitinib, CPL-409116, CS-12192, tofacitinib citrate, ISB-830, DV-1079, julemic acid, iberdomide, TAM-01, BML-258, brepocitinib, SDC-1801, SDC-1802, ICP-330, NTR-441, dalazatide, GSK-2646264, SKI-O-703, lanraplenib (GS-9876), GNS-1653, HMPL-523, RSLV-132, interleukin-2 follow-on biologic, interleukin-2 Anteluke, interking recombinant human interleukin-2, ILT-101, CUG-252, DZ-2002, PEGylated HLA-x (SLE), AC-0058, fenebrutinib, XNW-1011, tirabrutinib hydrochloride, branebrutinib, elsubrutinib, orelabrutinib, DWP-213388, INV-103, R-salbutamol sulphate, anchorins, NIK-SMI1, X-6, INV-17, Oshadi D, baricitinib, upadacitinib, filgotinib, itacitinib, INCB-54707, delgocitinib, DWP-212525, CKD-971, as mometasone, betamethasone, forigerimod, anandamide, DCB-SLE1, arsenic trioxide, tairuimide, TV-4710 (edratide), allogeneic human umbilical cord-derived mesenchymal stem cell therapy (hUC-MSCs), LC-200, BI-705564, SM-934, GX-101, TXR-712, TXR-711, CIT-013, MHV-370, Panzyga®, TPX-6001, TPX-7001, artenimol, and AMG-592, or a pharmaceutically acceptable salt of any of the foregoing, or any combination thereof.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The method of claim 19, wherein the MK2-mediated disease or disorder is an autoimmune disorder, a chronic inflammatory disorder, an acute inflammatory disorder, an auto-inflammatory disorder, a fibrotic disorder, a metabolic disorder, a neoplastic disorder, a cardiovascular disorder, or a cerebrovascular disorder.

38. The method of claim 37, wherein the MK2-mediated disease or disorder is selected from inflammatory bowel disease, psoriasis, psoriatic arthritis, rheumatoid arthritis, glomerulonephritis, mixed connective tissue disease (MCTD), dermatomyositis, polymyositis, systemic sclerosis, antineutrophil cytoplasmic antibody-associated vasculitis, anti-phospholipid syndrome, autoimmune hemolytic anemia, macrophage activation syndrome driven inflammatory anemia, IgA nephropathy, type I diabetes, non-alcoholic steatohepatitis, and Sjogren's syndrome.
